(19) United States
(12) Patent Application Publication
(10) Pub. No.: US 2026/0137703 A1
(43) Pub. Date: May 21, 2026

(54) 2-AMINO-PYRIDO[2,3-D]PYRIMIDIN-7(8H)-ONE AND 7-AMINO-1-PYRIMIDO[4,5-D]PYRIMIDIN-2(1 H)-ONE DERIVATIVES AS EGFR INHIBITORS FOR THE TREATMENT OF CANCER

(71) Applicant: Cancer Research Technology Limited, London (GB)

(72) Inventors: Michael J. Waring, Newcastle upon Tyne (GB); Hannah L. Stewart, Newcastle upon Tyne (GB); Matthew P. Martin, Newcastle upon Tyne (GB); Islam H. Al-Khawaldeh, Newcastle upon Tyne (GB); Jack T. Herbert, Newcastle upon Tyne (GB); Steven Howard, Cambridge (GB); David Geoffrey Twigg, Cambridge (GB); John Walter Liebeschuetz, Cambridge (GB); Jeffrey David St. Denis, Cambridge (GB)

(21) Appl. No.: 19/123,232

(22) PCT Filed: Oct. 19, 2023
(86) PCT No.: PCT/GB2023/052721

§ 371 Date: Apr. 22, 2025

(30) Foreign Application Priority Data
Nov. 2, 2022 (GB) 2216303.4
Jan. 7, 2023 (GB) 2300691.9
Apr. 28, 2023 (GB) 2306370.4

(51) Int. Cl. A61K 31/635; A61K 31/437; A61K 31/519; A61K 31/529; A61K 31/5377; A61K 31/551; C07D 471/04; C07D 471/18; C07D 487/04; C07D 498/18; C07D 513/18

(52) U.S. Cl. CPC A61K 31/635; A61K 31/437; A61K 31/519; A61K 31/529; A61K 31/5377; A61K 31/551; C07D 471/04; C07D 471/18; C07D 487/04; C07D 498/18; C07D 513/18

(57) ABSTRACT

Provided are compounds of the Formula I, and salts, hydrates and solvates thereof:

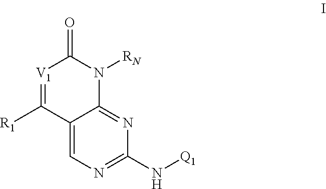

wherein R₁, V₁, R_{N} and Q₁ are each as defined in the specification. The compounds are inhibitors of EGFR, including mutated forms of EGFR (for example EGFR comprising a T790M mutation, a deletion in exon 19 (e.g. A740-A750), an exon 20 insertion, a mutation at L858R and/or a C797S mutation) that confer resistance to existing EGFR inhibitors.

### INTRODUCTION

The present invention relates to novel therapeutic compounds. More specifically, the present invention relates to novel therapeutic compounds that are inhibitors of epidermal growth factor receptor (EGFR). The present invention also relates to pharmaceutical compositions comprising the novel therapeutic compounds defined herein, to processes for synthesising these compounds and to their use for the treatment of diseases and/or conditions in which EGFR activity is implicated including, but not limited to, the treatment and/or prevention of proliferative disorders (e.g. cancer).

### BACKGROUND OF THE INVENTION

EGFR (Epidermal Growth Factor Receptor) is a receptor tyrosine kinase in the erbB family (which also includes erbB2, erbB3 and erbB4). It controls a number of downstream signalling pathways in cells by binding a ligand, such as the epidermal growth factor (EGF).

Binding of the ligand induces homodimerisation or heterodimerisation with other family members, which brings about autophosphorylation mediated by the EGFR kinase domain. This process triggers a signal transduction cascade.

EGFR signalling plays a key role in cellular proliferation, survival and suppression of apoptosis. These pathways can become disordered by overexpression or amplification of ligands or receptor, or through genetic alterations in EGFR. Aberrant EGFR signalling can be a key driver for oncogenic transformation, and tumour cell proliferation, invasion and metastasis. As an example, non-small cell lung cancer (NSCLC) patients frequently have activating mutations in EGFR, most commonly an in-frame deletion of Exon19, an L858R point mutation and missense mutations in Exon21 (Cancer Discovery, 2016, 6, 601).

Inhibitors of EGFR kinase activity, such as erlotinib, gefitinib, afatinib and osimertinib are effective treatments for EGFR mutated non-small cell lung cancer (Lancet Oncol. 2010, 11, 121; Lancet Oncol. 2016, 17, 577; J. Oncol. Pharm. Pract. 2020, 26, 1452; Lancet Oncol. 2011, 12, 735). Critical to the successful treatment is the selectivity of drugs for the mutated forms of EGFR relative to the wild-type since wild-type inhibition is associated with dose limiting side effects, such as skin rash and diarrhoea.

In almost all patients, treatment with first- and second-generation inhibitors, such as erlotinib, gefitinib and afatinib, leads to drug resistance after an average of 10-12 months (Lancet Oncol. 2010, 1, 121; Lancet Oncol. 2016 17, 577; Lancet Oncol. 2011, 12, 735). Most commonly, this resistance is due to a secondary mutation in the EGFR kinase domain T790M (J. Thorac. Oncol. 2009, 4, 1), which reduces the receptor's affinity for first- and second-generation drugs and increases its affinity for ATP (Proc. Natl. Acad. Sci. 2008, 105, 2070).

Third-generation EGFR inhibitors, such as osimertinib, have been developed to inhibit the T790M mutated forms of EGFR and have been shown to be active in both resistance mutant and activating mutant tumours and have become widely used in both first- and second-line treatment (N. Engl. J. Med. 2015, 372, 1689; N. Engl. J. Med. 2018, 378, 113). Osimertinib is a covalent inhibitor of EGFR that targets C797 (J. Med. Chem. 2014, 57, 8249). Resistance to third-generation therapies such as osimertinib develops with a duration of ca. 10 months. Commonly this resistance is attributable to mutations in the kinase domain that hinder the covalent binding, such as C797S (Brit. J. Cancer 2019, 121, 725).

There are no current treatments for cancers that are resistant to third-generation inhibitors. Hence, there is a high unmet need for inhibitors of activated forms of EGFR that harbour mutations, such as C797S that hinder the covalent binding of osimertinib, alongside other mutations such as Exon19Del and L858R, both with and without T790M, but with selectivity over the wild-type form.

The present invention was devised with the foregoing in mind.

### SUMMARY OF THE INVENTION

In one aspect, the present invention provides a compound of Formula I as defined herein, and/or a pharmaceutically acceptable salt, hydrate or solvate thereof.

In another aspect, the present invention provides a pharmaceutical composition which comprises a compound of Formula I as defined herein, or a pharmaceutically acceptable salt, hydrate or solvate thereof, and one or more pharmaceutically acceptable excipients.

In another aspect, the present invention provides a compound of Formula I as defined herein, or a pharmaceutically acceptable salt, hydrate or solvate thereof, or a pharmaceutical composition as defined herein, for use in therapy.

In another aspect, the present invention provides a compound of Formula I as defined herein, or a pharmaceutically acceptable salt, hydrate or solvate thereof, or a pharmaceutical composition as defined herein, for use in the treatment of a disease or condition in which EGFR activity is implicated.

In another aspect, the present invention provides a compound of Formula I as defined herein, or a pharmaceutically acceptable salt, hydrate or solvate thereof, or a pharmaceutical composition as defined herein, for use in the treatment of a disease or condition associated with aberrant activity of EGFR.

In another aspect, the present invention provides a compound of Formula I as defined herein, or a pharmaceutically acceptable salt, hydrate or solvate thereof, or a pharmaceutical composition as defined herein, for use in the treatment of proliferative disorders (e.g. cancer).

In another aspect, the present invention provides a compound of Formula I as defined herein, or a pharmaceutically acceptable salt, hydrate or solvate thereof, or a pharmaceutical composition as defined herein, for use in the treatment of cancer.

In another aspect, the present invention provides the use of a compound of Formula I as defined herein, or a pharmaceutically acceptable salt, hydrate or solvate thereof, in the manufacture of a medicament for use in the treatment of a disease or condition in which EGFR activity is implicated.

In another aspect, the present invention provides the use of a compound of Formula I as defined herein, or a pharmaceutically acceptable salt, hydrate or solvate thereof, in the manufacture of a medicament for use in the treatment of a disease or condition associated with aberrant activity of EGFR.

In another aspect, the present invention provides the use of a compound of Formula I as defined herein, or a pharmaceutically acceptable salt, hydrate or solvate thereof, in the manufacture of a medicament for use in the treatment of proliferative disorders (e.g. cancer or benign neoplasms).

In another aspect, the present invention the use of a compound of Formula I as defined herein, or a pharmaceutically acceptable salt, hydrate or solvate thereof, in the manufacture of a medicament for use in the treatment of a cancer.

In another aspect, the present invention provides a method of treating a disease or condition in which EGFR activity is implicated, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a disease or condition associated with aberrant activity of EGFR, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a proliferative disorder (e.g. cancer or benign neoplasms), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of formula I, or a pharmaceutically acceptable salt, hydrate or solvate thereof, for use in the treatment of an EGFR positive cancer, optionally selected from head and neck cancer, brain cancer, breast cancer, colon cancer and/or lung cancer.

In another aspect, the present invention provides a compound of formula I, or a pharmaceutically acceptable salt, hydrate or solvate thereof, for use in the treatment of EGFR positive non-small cell lung cancer.

In another aspect, the present invention provides a compound of formula I, or a pharmaceutically acceptable salt, hydrate or solvate thereof, for use in the treatment of a cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides a compound of formula I or a pharmaceutically acceptable salt, hydrate or solvate thereof, for use in the treatment of non-small cell lung cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides a compound of formula I, or a pharmaceutically acceptable salt, hydrate or solvate thereof, for use in the treatment of a cancer resistant to treatment with a third generation EFGR inhibitor, e.g. osimertinib, lazertinib (YH25448), EGF816, olmutinib, PF-06747775, avitinib and/or rociletinib.

In another aspect, the present invention provides a compound of formula I, or a pharmaceutically acceptable salt, hydrate or solvate thereof, for use in the treatment of non-small cell lung cancer resistant to treatment with osimertinib.

In another aspect, the present invention provides the use of a compound of formula I, or a pharmaceutically acceptable salt, hydrate or solvate thereof, in the manufacture of a medicament for use in the treatment of an EGFR positive cancer, optionally selected from head and neck cancer, brain cancer, breast cancer, colon cancer and/or lung cancer.

In another aspect, the present invention provides the use of a compound of formula I, or a pharmaceutically acceptable salt, hydrate or solvate thereof, in the manufacture of a medicament for use in the treatment of EGFR positive non-small cell lung cancer.

In another aspect, the present invention provides the use of a compound of formula I, or a pharmaceutically acceptable salt, hydrate or solvate thereof, in the manufacture of a medicament for use in the treatment of a cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides the use of a compound of formula I or a pharmaceutically acceptable salt, hydrate or solvate thereof, in the manufacture of a medicament for use in the treatment of non-small cell lung cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides the use of a compound of formula I, or a pharmaceutically acceptable salt, hydrate or solvate thereof, in the manufacture of a medicament for use in the treatment of a cancer resistant to treatment with a third generation EFGR inhibitor, e.g. osimertinib, lazertinib (YH25448), EGF816, olmutinib, PF-06747775, avitinib and/or rociletinib.

In another aspect, the present invention provides the use of a compound of formula I, or a pharmaceutically acceptable salt, hydrate or solvate thereof, in the manufacture of a medicament for use in the treatment of non-small cell lung cancer resistant to treatment with osimertinib.

In another aspect, the present invention provides a method of treating an EGFR positive cancer, optionally selected from head and neck cancer, brain cancer, breast cancer, colon cancer and/or lung cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating EGFR positive non-small cell lung cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a cancer expressing a mutated form of EGFR (for example EGFR comprising a T790M mutation, a deletion in exon 19 (such as A740-A750), an exon 20 insertion, a mutation at L858R and/or a C797S mutation), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating non-small cell lung cancer expressing a mutated form of EGFR (for example EGFR comprising a T790M mutation, a deletion in exon 19 (such as A740-A750), an exon 20 insertion, a mutation at L858R and/or a C797S mutation), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a cancer resistant to treatment with a third generation EFGR inhibitor, e.g. osimertinib, lazertinib (YH25448), EGF816, olmutinib, PF-06747775, avitinib and/or rociletinib, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating non-small cell lung cancer resistant to treatment with osimertinib, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a combination treatment comprising a compound of Formula I, or a pharmaceutically acceptable salt, hydrate or solvate thereof, as defined herein, with one or more additional therapeutic agents.

In another aspect, the present invention provides processes for preparing compounds of Formula I as defined herein, or a pharmaceutically acceptable salt, hydrate or solvate thereof, as defined herein, with one or more additional therapeutic agents.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of Formula I”, “compounds of the invention” and the more general term “compounds” refer to and include any and all compounds described by and/or with reference to Formula I herein. It should also be understood that these terms encompasses all stereoisomers, i.e. cis and trans isomers, as well as optical isomers, i.e. R and S enantiomers, of such compounds, in substantially pure form and/or any mixtures of the foregoing in any ratio. This understanding extends to pharmaceutical compositions and methods of treatment that employ or comprise one or more compounds of the Formula I, either by themselves or in combination with additional agents.

Unless specified otherwise, atoms are referred to herein by their chemical symbol as appearing in the IUPAC periodic table of the Elements. For example, “C” refers to a carbon atom.

The term “(m-nC)” or “(m-nC) group” used alone or as a prefix, refers to any group having m to n carbon atoms.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For Example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(3-6C)cycloalkyl” means a hydrocarbon ring containing from 3 to 6 carbon atoms, for example, cyclopropyl, cyclobutyl, cyclopentyl, cyclohexyl, cycloheptyl or bicyclo[2.2.1]heptyl.

The term “halo” or “halogeno” refers to fluoro, chloro, bromo and iodo.

As used herein by themselves or in conjunction with another term or terms, “haloalkyl” and “haloalkyl group” refer to alkyl groups in which one or more hydrogen atoms are replaced by halogen atoms. Representative examples include, but are not limited to, —CF₃, —CHF₂, —CH₂F, —CF₂CF₃, —CHFCF₃, and —CH₂CF₃. Suitably, a haloalkyl group is selected from —CHF₂ and —CF₃, suitably —CF₃.

As used herein by themselves or in conjunction with another term or terms, “haloalkoxy” and “haloalkoxy group” refer to alkoxy groups (i.e. O-alkyl groups) in which one or more hydrogen atoms are replaced by halogen atoms. Representative examples include, but are not limited to, —OCF₃, —OCHF₂, —OCH₂F, and —OCF₂CF₃. Suitably, a haloalkyoxy group is selected from —OCHF₂ and —OCF₃, suitably —OCF₃.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as, but not limited to, oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydrooxathiolyl, tetrahydrooxazolyl, tetrahydro-oxadiazolyl, tetrahydrodioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as, but not limited to, tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bicyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

The term “heteroaryl” or “heteroaromatic” means an aromatic mono-, bi-, or polycyclic ring incorporating one or more (for example 14, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3b]-furanyl-, 2H-furo[3,2b]-pyranyl-, 5H-pyrido[2,3-d]-ooxazinyl-, 1H-pyrazolo[4,3-d]-oxazolyl, 4H-imidazo[4,5d]thiazolyl, pyrazino[2,3d]pyridazinyl, -imidazo[2,1b]thiazolyl, -imidazo[1,2b][1,2,4]-triazinyl. “Heteroaryl” also covers partially aromatic bi- or polycyclic ring systems wherein at least one ring is an aromatic ring and one or more of the other ring(s) is a nonaromatic, saturated or partially saturated ring, provided at least one ring contains one or more heteroatoms selected from nitrogen, oxygen or -sulfur-. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl and 6,8-dihydro-5H-[1,2,4]triazolo[4,3-a]pyrazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

This specification also makes use of several composite terms to describe groups comprising more than one functionality. Such terms will be understood by a person skilled in the art. For Example heterocyclyl(m-nC)alkyl comprises (m-nC)alkyl substituted by heterocyclyl.

The term “aryl(1-2C)alkyl” means an aryl group covalently attached to a (1-2C)alkylene group, both of which are defined herein. Examples of aryl-(1-2C)alkyl groups include benzyl, phenylethyl, and the like.

“Heteroaryl(1-3C)alkyl” means a heteroaryl group covalently attached to a (1-3C)alkylene group, both of which are defined herein. Examples of heteroaryl-alkyl groups include pyridin-3-ylmethyl, 2-(benzofuran-2-yl)ethyl, and the like.

“Heterocyclyl(1-2C)alkyl” means a heterocyclyl group covalently attached to a (1-2C)alkylene group, both of which are defined herein.

“(3-6C)cycloalkyl-(1-2C)alkyl” means a (3-6C)cycloalkyl group covalently attached to a (1-2C)alkylene group, both of which are defined herein.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

A wavy bond ( [symbol] ) is used herein to show a point of attachment.

The phrase “compound of the invention” means those compounds which are disclosed herein, both generically and specifically.

As used herein by itself or in conjunction with another term or terms, “pharmaceutically acceptable” refers to materials that are generally chemically and/or physically compatible with other ingredients (such as, for example, with reference to a formulation), and/or are generally physiologically compatible with the recipient (such as, for example, a subject) thereof.

As used herein by themselves or in conjunction with another term or terms, “subject(s)” and “patient(s)”, suitably refer to mammals, in particular humans.

### Compounds of the Invention

In a first aspect, a compound of formula I shown below, or a pharmaceutically acceptable salt thereof:

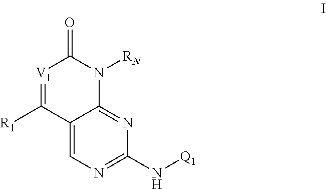

wherein:
R_{N} is selected from hydrogen, (1-6C)alkyl, (3-8C)cycloalkyl, (3-8C)cycloalkyl(1-4C)alkyl, aryl, aryl(1-4C)alkyl, carbon-linked heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in R_{N} is optionally substituted with one or more R_{NA} groups;
   wherein R_{NA} is selected from halo, nitro, cyano, oxo or a group of the formula:

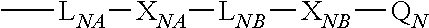

wherein:
   L_{NA} is absent or (1-3C)alkylene;
   X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)—N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-4C)alkyl;
   L_{NB} is absent or (1-4C)alkylene;
   X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —O—C(O)—N(R_{NB1})—, —N(R_{NB1})—C(O)—O—, —N(R_{NB2})—C(O)—NR_{NB1}—, SO₂N(R_{NB1})— or —N(R_{NB1})SO₂—, where R_{NB1} and R_{NB2} are independently selected from the group consisting of hydrogen or (1-4C)alkyl;
   Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, aryl, aryl(1-2C)alkyl, heterocyclyl, heterocyclyl(1-2C)alkyl, heteroaryl, or heteroaryl(1-4C)alkyl;
   and wherein Q_{N} is optionally substituted with one or more R_{NC} groups;
   and each R_{NC} group present is independently selected from the group consisting of hydroxy, cyano, oxo, halogen, (1-2C)alkyl, (1-2C)alkoxy, (3-6C)cycloalkyl, —NR_{NC1}R_{NC2} or —C(O)—R_{NC1},
   wherein R_{NC}1 and R_{NC2} are each independently hydrogen or (1-2C)alkyl;
R₁ is selected from hydrogen, (2C)alkynyl, (2C)alkenyl or (1-2C)alkyl;
V₁ is N or C—R₂;
R₂ is hydrogen, halo, cyano, or a group of the formula:

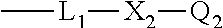

wherein:
   L₁ is absent or (1-3C)alkylene;
   X₂ is absent or is selected from the group consisting of —O—, —C(O)—, —NR₃ₐ—, —C(O)O, —O—C(O)—, —S(O)₀₋₂—, —C(O)—N(R₃ₐ)—, —N(R₃ₐ)—C(O)—, —O—C(O)—N(R₃ₐ)—, —N(R₃)—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-4C)alkyl;
   Q₂ is selected from the group consisting of hydrogen, (1-6C)alkyl, (3-8C)cycloalkyl, (3-8C)cycloalkyl(1-4C)alkyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkyl-alkyl, aryl, arylalkyl, heterocyclyl, heterocyclyl-alkyl, heteroaryl or heteroaryl-alkyl in Q₂ is optionally substituted with one or more R_{3c} groups; and
   each R_{3c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
   wherein R_{3d} and R₃ₑ are each independently hydrogen, (1-6C)alkyl, (3-6C)cycloalkyl, or (3-6C)cycloalkyl(1-2C)alkyl;
   and wherein any alkyl, alkoxy, cycloalkyl, cycloalkyl-alkyl, phenyl, heteroaryl or heterocyclyl group present in a R_{3c} group, or any alkyl, cycloalkyl, or cycloalkyl-alkyl group present in a R_{3d} or R_{3c} group, is optionally further substituted by one or more substituents independently selected from hydroxy, cyano, halogen, (1-2C)alkyl, —OR_{3f}, —NR_{3f}R_{3g} and —C(O)—R_{3f}, wherein R_{3f} and R₃₉ are both independently selected from hydrogen and (1-2C)alkyl; and
Q₁ is selected from hydrogen or:
   (i) a group of the formula:

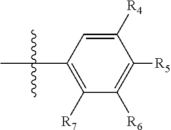

wherein:
   R₄ and R₆ are each independently selected from hydrogen, halo, cyano, nitro, or a group of the formula:

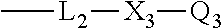

wherein:
L₂ is absent or (1-3C)alkylene;
X₃ is absent or is selected from the group consisting of —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂—, where R₄ₐ and R_{4b} are independently selected from the group consisting of hydrogen or (1-4C)alkyl;
Q₃ is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, —N(R₄ₑ)—S(O)₂R_{4d}, phenyl, 5 or 6-membered heteroaryl, or 4 to 6-membered heterocyclyl,
wherein R_{4d} and R₄ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{4c} group, or any alkyl, cycloalkyl or cycloalkyl-alkyl group present in a R_{4d} or R₄ₑ group, is optionally further substituted by one or more substituents independently selected from hydroxy, cyano, halogen, (1-2C)alkyl, —OR_{4f}, —NR₄ᵣR_{4g} and —C(O)—R₄ᵣ, wherein R₄ᵣ and R₄₉ are both independently selected from hydrogen and (1-2C)alkyl;
R₅ is:
a) hydrogen, halo, cyano;
b) a group of the formula:

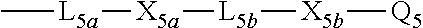

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, halogen, hydroxy, methyl, hydroxymethyl, methoxy, —CF₃, —OCF₃ or cyano;
or R₄ and R₅, or R₅ and R₆ are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

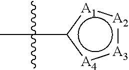

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkylalkyl, phenyl, benzyl, heterocyclyl, heterocyclylalkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 4 of A₁, A₂, A₃ and A₄ can be N;
(iii) a group of the formula:

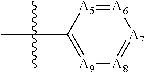

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that one or two of A₅, A₆, A₇, A₈ and A₉ can be N;
(iv) a group of the formula:

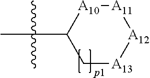

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

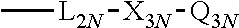

wherein:
L_{2N} is absent or (1-3C)alkylene;
X_{3N} is absent or is selected from the group consisting of:
   (i) —C(O)—, —S(O)₀₋₂— or —C(O)—N(R₄ₐ)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and each R_{4c} group present is as defined above;
R_{5N} is:
   a) hydrogen;
   b) a group of the formula:

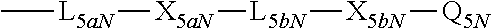

wherein:
L_{5aN} is absent or (1-3C)alkylene;
X_{5aN} is absent or is selected from the group consisting of:
   (i) —C(O)—, —S(O)₀₋₂—, —C(O)—N(R₅ₐ₁)— when L_{5aN} is absent; or
   (ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
   where R₅ₐ₁ and R₅ₐ₂ are as defined above;
L_{5bN} is absent or (1-4C)alkylene;
X_{5bN} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
   (i) hydrogen;
   (ii) (1-6C)alkyl, (3-8C)cycloalkyl, (3-8C)cycloalkyl(1-4C)alkyl, phenyl, phenyl(1-4C)alkyl, 5- or 6-membered heteroaryl, 5- or 6-membered heteroaryl(1-2C)alkyl;
   (iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
   and wherein Q_{5N} is optionally substituted with one or more R₅ₑ groups as defined above;
or R_{4N} and R_{5N}, or R_{5N} and R_{6N} are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
and with the proviso that one or two of A₁₀, A₁₁, A₁₂ and A₁₃ can be N;
   (v) a group of the formula:

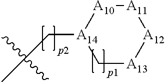

wherein:
p1, A₁₀, A₁₁, A₁₂, and A₁₃ are as defined above;
p2 is 1 to 6;
A₁₄ is C or N;
and with the proviso that one of A₁₀, A₁₁, A₁₂ and A₁₃ can be N;
   or:
R_{N} and one of R₄ or R₅ are optionally linked to form a linker group L.

In another aspect, the present invention provides a compound of formula I, or a pharmaceutically acceptable salt thereof:

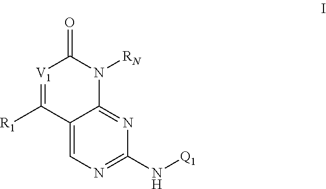

wherein:
R_{N} is selected from hydrogen, (1-6C)alkyl, (3-8C)cycloalkyl, (3-8C)cycloalkyl(1-4C)alkyl, aryl, aryl(1-4C)alkyl, carbon-linked heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in R_{N} is optionally substituted with one or more R_{NA} groups;
   wherein R_{NA} is selected from halo, nitro, cyano, oxo or a group of the formula:

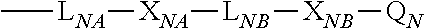

wherein
   L_{NA} is absent or (1-3C)alkylene;
   X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)—N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-4C)alkyl;
   L_{NB} is absent or (1-4C)alkylene;
   X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —O—C(O)—N(R_{NB1})—, —N(R_{NB1})—C(O)—O—, —N(R_{NB2})—C(O)—NR_{NB1}—, SO₂N(R_{NB1})— or —N(R_{NB1})SO₂—, where R_{NB1} and R_{NB2} are independently selected from the group consisting of hydrogen or (1-4C)alkyl;
   Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, aryl, aryl(1-2C)alkyl, heterocyclyl, heterocyclyl(1-2C)alkyl, heteroaryl, or heteroaryl(1-4C)alkyl;
   and wherein Q_{N} is optionally substituted with one or more R_{NC} groups; and each R_{NC} group present is independently selected from the group consisting of hydroxy, cyano, oxo, halogen, (1-2C)alkyl, (1-2C)alkoxy, (3-6C)cycloalkyl, —NR_{NC1}R_{NC2} or —C(O)—R_{NC1},
   wherein R_{NC}1 and R_{NC2} are each independently hydrogen or (1-2C)alkyl;
R₁ is selected from hydrogen, (2C)alkynyl, (2C)alkenyl or (1-2C)alkyl;
V₁ is N or C—R₂;
R₂ is hydrogen, halo, cyano, or a group of the formula:

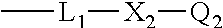

wherein:
   L₁ is absent or (1-3C)alkylene;
   X₂ is absent or is selected from the group consisting of —O—, —C(O)—, —NR₃ₐ—, —C(O)O, —O—C(O)—, —S(O)₀₋₂—, —C(O)—N(R₃ₐ)—, —N(R₃ₐ)—C(O)—, —O—C(O)—N(R₃ₐ)—, —N(R₃)—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-4C)alkyl;
   Q₂ is selected from the group consisting of hydrogen, (1-6C)alkyl, (3-8C)cycloalkyl, (3-8C)cycloalkyl(1-4C)alkyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkyl-alkyl, aryl, arylalkyl, heterocyclyl, heterocyclyl-alkyl, heteroaryl or heteroaryl-alkyl in Q₂ is optionally substituted with one or more R_{3c} groups; and
   each R_{3c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
   wherein R_{3d} and R₃ₑ are each independently hydrogen, (1-6C)alkyl, (3-6C)cycloalkyl, or (3-6C)cycloalkyl(1-2C)alkyl;
   and wherein any alkyl, alkoxy, cycloalkyl, cycloalkyl-alkyl, phenyl, heteroaryl or heterocyclyl group present in a R_{3c} group, or any alkyl, cycloalkyl, or cycloalkyl-alkyl group present in a R_{3d} or R₃ₑ group, is optionally further substituted by one or more substituents independently selected from hydroxy, cyano, halogen, (1-2C)alkyl, —OR_{3f}, —NR_{3f}R_{3g} and —C(O)—R_{3f}, wherein R_{3f} and R₃₉ are both independently selected from hydrogen and (1-2C)alkyl; and
Q₁ is selected from hydrogen or:
   (i) a group of the formula:

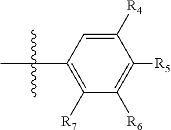

wherein:
   R₄, R₆ and R₇ are each independently selected from hydrogen, halo, cyano, nitro, or a group of the formula:

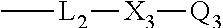

wherein:
L₂ is absent or (1-3C)alkylene;
X₃ is absent or is selected from the group consisting of —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂—, where R₄ₐ and R_{4b} are independently selected from the group consisting of hydrogen or (1-4C)alkyl;
Q₃ is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, —N(R₄ₑ)—S(O)₂R_{4d}, phenyl, 5 or 6-membered heteroaryl, or 4 to 6-membered heterocyclyl,
wherein R_{4d} and R₄ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R₄ₑ group, or any alkyl, cycloalkyl or cycloalkyl-alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —C(O)OR_{4f}, —OC(O)R_{4f}, —C(O)NR_{4f}R_{4g}, —NR₄₉C(O)R_{4f}, —S(O)₀₋₂—R_{4f} and —C(O)—R_{4f}, wherein R_{4f} and R₄₉ are both independently selected from hydrogen and (1-2C)alkyl;
R₅ is:
a) hydrogen, halo, cyano;
b) a group of the formula:

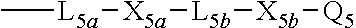

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

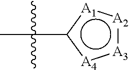

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkylalkyl, phenyl, benzyl, heterocyclyl, heterocyclylalkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 4 of A₁, A₂, A₃ and A₄ can be N;
(iii) a group of the formula:

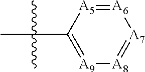

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that one or two of A₅, A₆, A₇, A₈ and A₉ can be N;
(iv) a group of the formula:

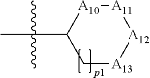

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

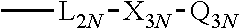

wherein:
L_{2N} is absent or (1-3C)alkylene;
X_{3N} is absent or is selected from the group consisting of:
   (i) —C(O)—, —S(O)₀₂— or —C(O)—N(R₄ₐ)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is as defined above;
R_{5N} is:
   a) hydrogen;
   b) a group of the formula:

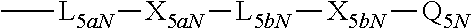

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-4C)alkylene;
X_{5bN} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-6C)alkyl, (3-8C)cycloalkyl, (3-8C)cycloalkyl(1-4C)alkyl, phenyl, phenyl(1-4C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R₅ₑ groups as defined above;
or R_{4N} and R_{5N}, or R_{5N} and R_{6N} are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
and with the proviso that one or two of A₁₀, A₁₁, A₁₂ and A₁₃ can be N;
(v) a group of the formula:

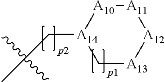

wherein:
p1, A₁₀, A₁₁, A₁₂, and A₁₃ are as defined above;
p2 is 1 to 6;
A₁₄ is C or N;
and with the proviso that one of A₁₀, A₁₁, A₁₂ and A₁₃ can be N;
   or:
R_{N} and one of R₄ or R₅ are optionally linked to form a linker group L.

In a particular group of compounds of the invention, R_{N} is not a directly aryl group that is optionally substituted, i.e. R_{N} is selected from hydrogen, (1-6C)alkyl, (3-8C)cycloalkyl, (3-8C)cycloalkyl(1-4C)alkyl, aryl(1-4C)alkyl, carbon-linked heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in R_{N} is optionally substituted with one or more R_{NA} groups;
wherein R_{NA} is selected from halo, nitro, cyano, oxo or a group of the formula:

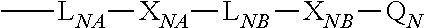

wherein:
L_{NA} is absent or (1-3C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-4C)alkyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —O—C(O)—N(R_{NB1})—, —N(R_{NB1})—C(O)—O—, —N(R_{NB2})—C(O)—NR_{NB1}—, —SO₂N(R_{NB1})— or —N(R_{NB1})SO₂—, where R_{NB1} and R_{NB2} are independently selected from the group consisting of hydrogen or (1-4C)alkyl;
Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, aryl, aryl(1-2C)alkyl, heterocyclyl, heterocyclyl(1-2C)alkyl, heteroaryl, or heteroaryl(1-4C)alkyl;
and wherein Q_{N} is optionally substituted with one or more R_{NC} groups;
and each R_{NC} group present is independently selected from the group consisting of hydroxy, cyano, oxo, halogen, (1-2C)alkyl, (1-2C)alkoxy, (3-6C)cycloalkyl, —NR_{NC1}R_{NC2} or —C(O)—R_{NC1},
wherein R_{NC}1 and R_{NC2} are each independently hydrogen or (1-2C)alkyl.

In a particular group of compounds of the invention, R_{N} and one of R₄ or R₅ are optionally linked to form a linker group L as defined herein.

Particular compounds of the invention include, for example, compounds of the formula I, or pharmaceutically acceptable salts, hydrates and/or solvates thereof, wherein, unless otherwise stated, each of R_{N}, R_{NA}, R₁, V₁, R₂, Q₁ and L each have any of the meanings defined hereinbefore or are as defined in any one of paragraphs (1) to (55) hereinafter:—
(1) R_{N} is selected from hydrogen, (1-5C)alkyl, (3-6C)cycloalkyl, (3-6C)cycloalkyl(1-4C)alkyl, phenyl, phenyl(1-4C)alkyl, carbon-linked 3- to 8-membered heterocyclyl, 3- to 8-membered heterocyclyl(1-4C)alkyl, 5- or 6-membered heteroaryl and [5- or 6-membered heteroaryl](1-4C)alkyl,
   wherein any alkyl, cycloalkyl, cycloalkylalkyl, phenyl, phenylalkyl, heterocyclyl, heterocyclylalkyl, heteroaryl or heteroarylalkyl in R_{N} is optionally substituted with one or more R_{NA} groups as defined above or in any one of paragraphs (4) to (10) below;
(2) R_{N} is selected from hydrogen, (1-4C)alkyl, (3-6C)cycloalkyl, (3-6C)cycloalkyl(1-4C)alkyl, phenyl, phenyl(1-4C)alkyl, carbon-linked 4- to 6-membered heterocyclyl, 4- to 6-membered heterocyclyl(1-4C)alkyl, 5- or 6-membered heteroaryl and [5- or 6-membered heteroaryl](1-4C)alkyl,
   wherein any alkyl, cycloalkyl, cycloalkylalkyl, aryl, arylalkyl, heterocyclyl, heterocyclylalkyl, heteroaryl or heteroarylalkyl in R_{N} is optionally substituted with one or more R_{NA} groups as defined above or in any one of paragraphs (4) to (10) below;
(3) R_{N} is selected from (1-4C)alkyl, (3-6C)cycloalkyl, (3-6C)cycloalkyl(1-2C)alkyl, phenyl, phenyl(1-2C)alkyl, carbon-linked 5- to 6-membered heterocyclyl, 5- to 6-membered heterocyclyl(1-2C)alkyl, 5- or 6-membered heteroaryl and [5- or 6-membered heteroaryl](1-2C)alkyl,
   wherein any alkyl, cycloalkyl, cycloalkylalkyl, aryl, arylalkyl, heterocyclyl, heterocyclylalkyl, heteroaryl or heteroarylalkyl in R_{N} is optionally substituted with one or more R_{NA} groups as defined above or in any one of paragraphs (4) to (10) below;
(4) R_{NA} is selected from halo, nitro, cyano, oxo or a group of the formula:

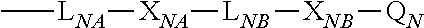

wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)—N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-2C)alkyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —C(O)—O—N(R_{NB1})—, —N(R_{NB1})—C(O)—O—, —N(R_{NB2})—C(O)—NR_{NB1}—, —SO₂N(R_{NB1})— or —N(R_{NB1})SO₂—, where R_{NB1} and R_{NB2} are independently selected from the group consisting of hydrogen or (1-2C)alkyl;
Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, phenyl, phenyl(1-2C)alkyl, 3- to 8-membered heterocyclyl, [3- to 8-membered heterocyclyl](1-2C)alkyl, 5- or 6-membered heteroaryl, or [5- or 6-membered heteroaryl](1-2C)alkyl;
and wherein Q_{N} is optionally substituted with one or more R_{NC} groups;
and each R_{NC} group present is independently selected from the group consisting of hydroxy, cyano, oxo, halogen, (1-2C)alkyl, (1-2C)alkoxy, (3-6C)cycloalkyl, —NR_{NC1}R_{NC2} or —C(O)—R_{NC1},
wherein R_{NC}1 and R_{NC2} are each independently hydrogen or (1-2C)alkyl;
(5) R_{NA} is selected from halo, nitro, cyano, oxo or a group of the formula:

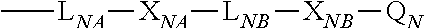

wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)—N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-2C)alkyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —O—C(O)—N(R_{NB1})—, —N(R_{NB1})—C(O)—O—, —N(R_{NB2})—C(O)—NR_{NB1}—, —SO₂N(R_{NB1})— or —N(R_{NB1})SO₂—, where R_{NB1} and R_{NB2} are independently selected from the group consisting of hydrogen or (1-2C)alkyl;
Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, phenyl, phenyl(1-2C)alkyl, 3- to 8-membered heterocyclyl, [3- to 8-membered heterocyclyl](1-2C)alkyl, 5- or 6-membered heteroaryl, or [5- or 6-membered heteroaryl](1-2C)alkyl;
and wherein Q_{N} is optionally substituted with one or more R_{NC} groups;
and each R_{NC} group present is independently selected from the group consisting of hydroxy, cyano, oxo, halogen, (1-2C)alkyl or (1-2C)alkoxy;
(6) R_{NA} is selected from halo, nitro, cyano, oxo or a group of the formula:

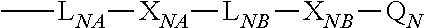

wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)—N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-2C)alkyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —O—C(O)—N(R_{NB1})—, —N(R_{NB1})—C(O)—O—, —N(R_{NB2})—C(O)—NR_{NB1}—, —SO₂N(R_{NB1})— or —N(R_{NB1})SO₂—, where R_{NB1} and R_{NB2} are independently selected from the group consisting of hydrogen or (1-2C)alkyl;
Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, phenyl, phenyl(1-2C)alkyl, 3- to 8-membered heterocyclyl, [3- to 8-membered heterocyclyl](1-2C)alkyl, 5- or 6-membered heteroaryl, or [5- or 6-membered heteroaryl](1-2C)alkyl;
(7) R_{NA} is selected from halo, nitro, cyano, oxo or a group of the formula:

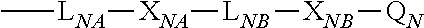

wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})— or —N(R_{NA1})—C(O)—, where R_{NA1} is hydrogen or methyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —SO₂N(R_{NB1})— or —N(R_{NB1})SO₂—, where R_{NB1} and R_{NB2} are independently selected from the group consisting of hydrogen or methyl;
Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, phenyl, phenyl(1-2C)alkyl, 3- to 8-membered heterocyclyl, [3- to 8-membered heterocyclyl](1-2C)alkyl, 5- or 6-membered heteroaryl, or [5- or 6-membered heteroaryl](1-2C)alkyl;
(8) R_{NA} is selected from halo, nitro, cyano, oxo or a group of the formula:

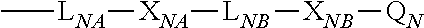

wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})— or —N(R_{NA1})—C(O)—, where R_{NA1} is hydrogen or methyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —SO₂N(R_{NB1})— or —N(R_{NB1})SO₂—, where R_{NB1} and R_{NB2} are independently selected from the group consisting of hydrogen or methyl;
Q_{N} is selected from the group consisting of hydrogen, (1-6C)alkyl, (2-6C)alkenyl, (3-6C)cycloalkyl, (3-6C)cycloalkyl(1-2C)alkyl, phenyl, phenyl(1-2C)alkyl, 3- to 6-membered heterocyclyl, [3- to 6-membered heterocyclyl](1-2C)alkyl, 5- or 6-membered heteroaryl, or [5- or 6-membered heteroaryl](1-2C)alkyl;
(9) R_{NA} is selected from halo, nitro, cyano, oxo or a group of the formula:

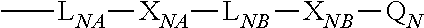

wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})— or —N(R_{NA1})—C(O)—, where R_{NA1} is hydrogen or methyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —SO₂N(R_{NB1})— or —N(R_{NB1})SO₂—, where R_{NB1} and R_{NB2} are independently selected from the group consisting of hydrogen or methyl;
Q_{N} is selected from the group consisting of hydrogen, (1-6C)alkyl, (2-6C)alkenyl, (3-6C)cycloalkyl, 3- to 6-membered heterocyclyl, [3- to 6-membered heterocyclyl](1-2C)alkyl, 5- or 6-membered heteroaryl, or [5- or 6-membered heteroaryl](1-2C)alkyl;
(10) R_{NA} is selected from halo, nitro, cyano, oxo or a group of the formula:

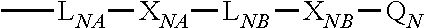

wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})— or —N(R_{NA1})—C(O)—, where R_{NA1} is hydrogen or methyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —SO₂N(R_{NB1})— or —N(R_{NB1})SO₂—, where R_{NB1} and R_{NB2} are independently selected from the group consisting of hydrogen or methyl;
Q_{N} is selected from the group consisting of hydrogen, (1-6C)alkyl, (2-6C)alkenyl, (3-6C)cycloalkyl, 5- or 6-membered heteroaryl, or [5- or 6-membered heteroaryl](1-2C)alkyl;
(11) R₁ is selected from ethynyl or ethenyl;
(12) R₁ is ethynyl;
(13) R₁ is ethenyl;
(14) V₁ is N;
(15) V₁ is C—R₂;
(16) R₂ is hydrogen, halo, cyano, or a group of the formula

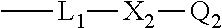

wherein:
L₁ is absent or (1-2C)alkylene;
X₂ is absent or is selected from the group consisting of —O—, —C(O)—, —NR₃ₐ—, —C(O)O, —O—C(O)—, —S(O)₀₋₂—, —C(O)—N(R₃ₐ)—, —N(R₃ₐ)—C(O)—, —O—C(O)—N(R₃ₐ)—, —N(R₃)—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 8-membered heterocyclyl, [3- to 8-membered heterocyclyl](1-2C)alkyl, 5- or 6-membered heteroaryl and [5- to 6-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, C(O)—NR_{3d}R₃ₑ, —N(R₃ₑ)C(O)—R_{3d}, —S(O)₀₋₂R_{3d}—, —S(O)₂NR_{3d}R₃ₑ, N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen, (1-4C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{3c} group, or any alkyl, cycloalkyl, or cycloalkyl-alkyl group present in a R_{3d} or R₃ₑ group, is optionally further substituted by one or more substituents independently selected from hydroxy, cyano, halogen, (1-2C)alkyl, —OR_{3f}, —NR_{3f}R_{3g} and —C(O)—R_{3f}, wherein R_{3f} and R₃₉ are both independently selected from hydrogen and (1-2C)alkyl;
(17) R₂ is hydrogen, halo, cyano, or a group of the formula

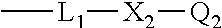

wherein:
L₁ is absent or (1-2C)alkylene;
X₂ is absent or is selected from the group consisting of —O—, —C(O)—, —NR₃ₐ—, —C(O)—, —O—C(O)—, S(O)₀₋₂—, —C(O)—N(R₃ₐ)—, —N(R₃ₐ)—C(O)—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 8-membered heterocyclyl, [3- to 8-membered heterocyclyl](1-2C)alkyl, 5- or 6-membered heteroaryl and [5- to 6-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, cycloalkyl, cycloalkyl-alkyl, phenyl, heteroaryl or heterocyclyl group present in a R_{3c} group, or any alkyl group present in a R_{3d} or R₃ₑ group, is optionally further substituted by one or more substituents independently selected from hydroxy, cyano, halogen, (1-2C)alkyl, or —OR_{3f}, wherein R_{3f} is selected from hydrogen and (1-2C)alkyl;
(18) R₂ is hydrogen, cyano, or a group of the formula

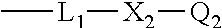

wherein:
L₁ is absent or (1-2C)alkylene;
X₂ is absent or is selected from the group consisting of —O—, —C(O)—, —NR₃ₐ—, —C(O)O, —O—C(O)—, —S(O)₀₋₂—, —C(O)—N(R₃ₐ)—, —N(R₃ₐ)—C(O)—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- or 6-membered heteroaryl and [5- to 6-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, cycloalkyl, cycloalkyl-alkyl, phenyl, heteroaryl or heterocyclyl group present in a R_{3c} group is optionally further substituted by one or more substituents independently selected from hydroxy, cyano, halogen, (1-2C)alkyl, or —OR_{3f}, wherein R_{3f} is selected from hydrogen and (1-2C)alkyl;
(19) R₂ is hydrogen, cyano, or a group of the formula

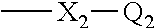

wherein:
X₂ is absent or is selected from the group consisting of —O—, —C(O)—, —NR₃ₐ—, —C(O)O, —O—C(O)—, —S(O)₀₋₂—, —C(O)—N(R₃ₐ)—, —N(R₃ₐ)—C(O)—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- or 6-membered heteroaryl and [5- to 6-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, —NR_{3d}R₃ₑ, phenyl, 5 or 6-membered heteroaryl, or 4 to 6-membered heterocyclyl, wherein R_{3d} and R₃ₑ are each independently hydrogen or (1-2C)alkyl; and wherein any alkyl, alkoxy, phenyl, heteroaryl or heterocyclyl group present in a R₃ₑ group, is optionally further substituted by one or more substituents independently selected from hydroxy, cyano, halogen, (1-2C)alkyl, or —OR_{3f}, wherein R_{3f} is selected from hydrogen and (1-2C)alkyl;
(20) R₂ is hydrogen, cyano, or a group of the formula

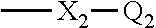

wherein:
X₂ is absent or is selected from the group consisting of —O—, —C(O)—, —NR₃ₐ—, —C(O)—O—, —S(O)₀₋₂—, —C(O)—N(R₃ₐ)—, —N(R₃ₐ)—C(O)—, —SO₂N(R₃ₐ)— or —N(R₃ₐ)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- or 6-membered heteroaryl and [5- to 6-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, phenyl, 5 or 6-membered heteroaryl, or 4 to 6-membered heterocyclyl,
and wherein any alkyl, alkoxy, phenyl, heteroaryl or heterocyclyl group present in a R_{3c} group, is optionally further substituted by one or more substituents independently selected from hydroxy or halogen;
(21) R₂ is hydrogen, cyano, or a group of the formula

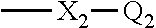

wherein:
X₂ is absent or is selected from the group consisting of —NR₃ₐ—, —C(O)—O—, —C(O)—N(R₃ₐ)— or —N(R₃ₐ)—C(O)—, where R₃ₐ is selected from the group consisting of hydrogen or methyl;
Q₂ is selected from the group consisting of hydrogen, (1-3C)alkyl, (3-6C)cycloalkyl, phenyl, phenyl(1-4C)alkyl, 5- or 6-membered heteroaryl and [5-to 6-membered heteroaryl](1-2C)alkyl, wherein any alkyl, cycloalkyl, phenyl, phenylalkyl, heteroaryl or heteroaryl-alkyl in Q₂ is optionally substituted with one or more R_{3c} groups; and
each R_{3c} group present is independently selected from the group consisting of hydroxy, cyano, halogen, (1-4C)alkyl, (1-4C)alkoxy, or 5 or 6-membered heteroaryl;
(22) Q₁ is selected from hydrogen or:
   (i) a group of the formula:

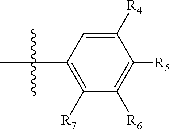

wherein:
R₄ and R₆ are each independently selected from hydrogen, halo, cyano, or a group of the formula:

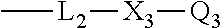

wherein:
L₂ is absent or (1-3C)alkylene;
X₃ is absent or is selected from the group consisting of —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl; and wherein any alkyl, alkoxy, cycloalkyl, cycloalkyl-alkyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR₄ᵣR_{4g} and —C(O)—R₄ᵣ, wherein R₄ᵣ and R₄₉ are both independently selected from hydrogen and (1-2C)alkyl;
R₅ is:
   a) hydrogen, halo, cyano; or
   b) a group of the formula:

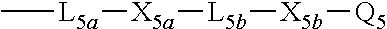

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-4C)alkyl, (1-4C)alkoxy, (3-6C)cycloalkyl (including spiro-fused (3-6C)cycloalkyl), or (3-6C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, halogen, hydroxy, methyl, hydroxymethyl, methoxy, or cyano;
or R₄ and R₅, or R₅ and R₆ are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

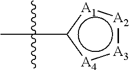

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N}; and
   (iii) 1 to 4 of A₁, A₂, A₃ and A₄ can be N;
(iii) a group of the formula:

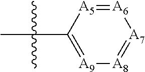

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that one or two of A₅, A₆, A₇, A₈ and A₉ can be N;
(iv) a group of the formula:

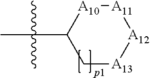

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

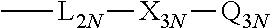

wherein:
L_{2N} is absent or (1-3C)alkylene;
X_{3N} is absent or is selected from the group consisting of:
   (i) —C(O)—, —S(O)₀₋₂— or —C(O)—N(R₄ₐ)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-4C)alkyl, (3-6C)cycloalkyl, (3-6C)cycloalkyl(1-2C)alkyl, phenyl, phenyl(1-4C)alkyl, heterocyclyl, heterocyclyl(1-2C)alkyl, heteroaryl and heteroaryl(1-2C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is as defined above;
R_{5N} is:
   a) hydrogen;
   b) a group of the formula:

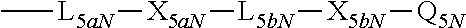

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-4C)alkylene;
X_{5bN} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-6C)alkyl, (3-8C)cycloalkyl, (3-8C)cycloalkyl(1-4C)alkyl, phenyl, phenyl(1-4C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R₅ₑ groups as defined above;
or R_{4N} and R_{5N}, or R_{5N} and R_{6N} are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
and with the proviso that one or two of A₁₀, A₁₁, A₁₂ and A₁₃ can be N;
(v) a group of the formula:

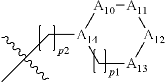

wherein:
p1, A₁₀, A₁₁, A₁₂, and A₁₃ are as defined above;
p2 is 1 to 5;
A₁₄ is C or N;
and with the proviso that one of A₁₀, A₁₁, A₁₂ and A₁₃ can be N;
or:
   R_{N} and R₄ are optionally linked to form a linker group L, as defined in any one of paragraphs (27) to (31) below.
(23) Q₁ is selected from hydrogen or:
   (i) a group of the formula:

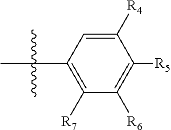

wherein:
R₄ and R₆ are each independently selected from hydrogen, halo, cyano, or a group of the formula:

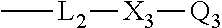

wherein:
L₂ is absent or (1-3C)alkylene;
X₃ is absent or is selected from the group consisting of —O—, —NR₄ₐ—, —C(O)—O—, —O—C(O)—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclyl-alkyl, heteroaryl or heteroaryl-alkyl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl; and wherein any alkyl, alkoxy, cycloalkyl, cycloalkyl-alkyl group present in a R_{4c} group, or any alkyl group present in a R_{4d} or R₄ₑ group, is optionally further substituted by one or more substituents independently selected from hydroxy, cyano, halogen, R_{4f}, —OR_{4f}, —NR_{4f}R_{4g} and —C(O)—R_{4f}, wherein R_{4f} and R₄₉ are both independently selected from hydrogen or (1-2C)alkyl;
R₅ is:
   a) hydrogen, halo, cyano; or
   b) a group of the formula:

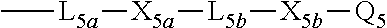

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

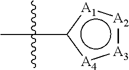

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

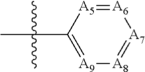

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

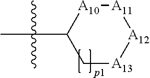

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

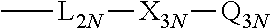

wherein:
L_{2N} is absent or (1-3C)alkylene;
X_{3N} is absent or is selected from the group consisting of:
   (i) —C(O)—, —S(O)₀₋₂— or —C(O)—N(R₄ₐ)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-6C)alkyl, (3-6C)cycloalkyl, (3-6C)cycloalkyl(1-2C)alkyl, heterocyclyl, heterocyclyl(1-2C)alkyl, heteroaryl and heteroaryl(1-2C)alkyl, wherein any alkyl, cycloalkyl, cycloalkylalkyl, heterocyclyl, heterocyclylalkyl, heteroaryl or heteroarylalkyl in Q₃ is optionally substituted with one or more R_{4c} groups; and
each R_{4c} group present is as defined above;
R_{5N} is
   a) hydrogen;
   b) a group of the formula:

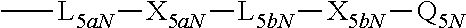

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R₅ₑ groups as defined above;
and with the proviso that one or two of A₁₀, A₁₁, A₁₂ and A₁₃ can be N;
(v) a group of the formula:

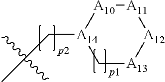

wherein:
p1, A₁₀, A₁₁, A₁₂, and A₁₃ are as defined above;
p2 is 1 to 5;
A₁₄ is C or N;
and with the proviso that one of A₁₀, A₁₁, A₁₂ and A₁₃ can be N;
or:
R_{N} and R₄ are optionally linked to form a linker group L, as defined in any one of paragraphs (27) to (31) below.
(24) Q₁ is selected from hydrogen or:
   (i) a group of the formula:
      R₄

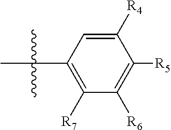

wherein:
R₄ and R₆ are each independently selected from hydrogen, or a group of the formula:

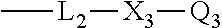

wherein:
L₂ is absent or (1-3C)alkylene;
X₃ is absent or is selected from the group consisting of —O—, —NR₄ₐ—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, phenyl, 3 to 8-membered heterocyclyl, or 5 or 6-membered heteroaryl, wherein any alkyl, cycloalkyl, cycloalkylalkyl, phenyl, heterocyclyl, or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
R₅ is:
   a) hydrogen, halo, cyano; or
   b) a group of the formula:

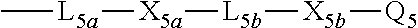

wherein:
L₅ₐ is absent or (1-3C)alkylene;
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

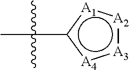

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

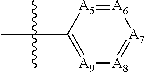

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

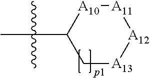

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

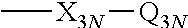

wherein:
X_{3N} is absent or is selected from the group consisting of: —C(O)—, —S(O)₀₋₂— or —C(O)—N(R₄ₐ)—:
   wherein R₄ₐ is as defined above;
Q_{3N} is selected from the group consisting of hydrogen, (1-4C)alkyl, (3-6C)cycloalkyl, (3-6C)cycloalkyl(1-2C)alkyl, heterocyclyl, heteroaryl and, wherein any alkyl, cycloalkyl, cycloalkylalkyl, heterocyclyl or heteroaryl in Q₃ is optionally substituted with one or more R_{4c} groups; and
each R_{4c} group present is as defined above; R_{5N} is
   a) hydrogen;
   b) a group of the formula:

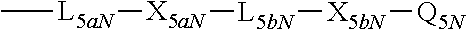

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

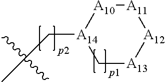

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or:
(25) R_{N} and R₄ are optionally linked to form a linker group L, as defined in any one of paragraphs (27) to (31) below; Q₁ is selected from hydrogen or:
   (i) a group of the formula:

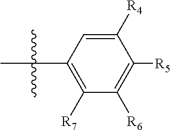

wherein:
R₄ and R₆ are each independently selected from hydrogen, or a group of the formula:

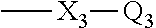

wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, phenyl, 3 to 8-membered heterocyclyl, or 5 or 6-membered heteroaryl, wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d}, wherein R_{4d} and R₄ₑ are each independently hydrogen or (1-2C)alkyl;
R₅ is:
   a) hydrogen, halo, cyano; or
   b) a group of the formula:

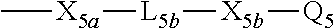

wherein:
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

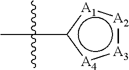

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

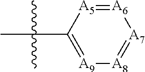

wherein:
A₅ is selected from N or CH;
A₇ is selected from N or CR₄;
A₇ is selected from N or CR₆;
A₈ is selected from N or CR₇;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

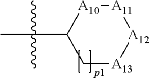

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is
   a) hydrogen;
   b) a group of the formula:

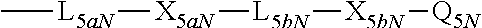

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

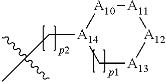

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or:
   R_{N} and R₄ are optionally linked to form a linker group L, as defined in any one of paragraphs (27) to (31) below.
(27) L is a linker group that separates the N atom to which R_{N} is attached and the carbon atom to which R₄ is attached by 5, 6 or 7 atoms, or 6, 7 or 8 bond lengths;
(28) L is a linker group that separates the N atom to which R_{N} is attached and the carbon atom to which R₄ is attached by 5 or 6 atoms, or 6 or 7 bond lengths;
(29) L is a linker group of the formula:

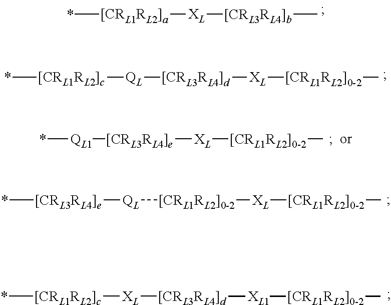

wherein:
   * denotes the point of attachment to the N atom in formula I;
   R_{L1}, R_{L2}, R_{L3} and R_{L4} are, at each occurrence, independently selected from hydrogen or (1-2C)alkyl;
   X_{L} or X_{L1} is absent or selected from —R_{L5}C=CR_{L6}—, ethynylene, —O—, —N(R_{LN})—, —C(O)—N(R_{LN})—, —N(R_{LN})—C(O)—, —C(O)—O—, —O—C(O)—, —S(O)₀₋₂—, —O—C(O)—N(R_{LN})—, —N(R_{LN})—C(O)—O—, —N(R_{LN1})—C(O)—N(R_{LN})—, —SO₂N(R_{LN})— or —N(R_{LN})SO₂—, wherein R_{L5} and R_{L6} are, at each occurrence, independently selected from hydrogen or (1-2C)alkyl, and R_{LN} and R_{LN1} are each independently selected from hydrogen or (1-2C)alkyl;
   Q_{L} is selected from a (4-6C)cycloalkyl, a 4 to 7 membered heterocyclyl or a 5 or 6-membered heteroaryl ring;
   Q_{L1} is selected from a (4-6C)cycloalkyl, a carbon-linked 4 to 7 membered heterocyclyl or a carbon-linked 5 or 6-membered heteroaryl ring;
   a is 1-6;
   b is 0-6;
   c is 1-6;
   d is 0-6; and
   e is 1-6;
and wherein Q_{L} and Q_{L1} are optionally substituted by halo, cyan, hydroxy, (1-2C)alkyl, (1-2C)alkoxy, amino, (1-2C)alkylamino, or [di-(1-2C)alkyl]amino;
(30) L is a linker group of the formula:

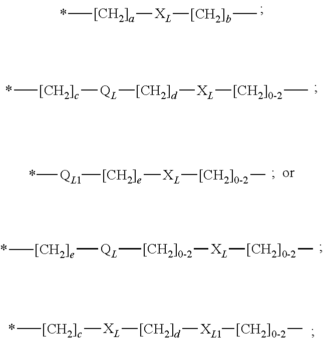

wherein:
   * denotes the point of attachment to the N atom in formula I;
   X_{L} or X_{L1} is absent or selected from —R_{L5}C=CR_{L6}—, ethynylene, —O—, —N(R_{LN})—, —C(O)—N(R_{LN})—, —N(R_{LN})—C(O)—, —C(O)—O—, —O—C(O)—, —S(O)₀₋₂—, —O—C(O)—N(R_{LN})—, —N(R_{LN})—C(O)—O—, —N(R_{LN1})—C(O)—N(R_{LN})—, —SO₂N(R_{LN})— or —N(R_{LN})SO₂—, wherein R_{L5} and R_{L6} are, at each occurrence, independently selected from hydrogen or (1-2C)alkyl, and R_{LN} and R_{LN}1 are each independently selected from hydrogen or (1-2C)alkyl;
   Q_{L} is selected from a (4-6C)cycloalkyl, a 4 to 7 membered heterocyclyl or a 5 or 6-membered heteroaryl ring;
   Q_{L}1 is selected from a (4-6C)cycloalkyl, a carbon-linked 4 to 7 membered heterocyclyl or a carbon-linked 5 or 6-membered heteroaryl ring;
   a is 1-5;
   b is 0-3;
   c is 1-3;
   d is 0-3; and
   e is 1-4;
and wherein Q_{L} and Q_{L}1 are optionally substituted by halo, cyan, hydroxy, (1-2C)alkyl, (1-2C)alkoxy, amino, (1-2C)alkylamino, or [di-(1-2C)alkyl]amino;
(31) L is a linker group of the formula:

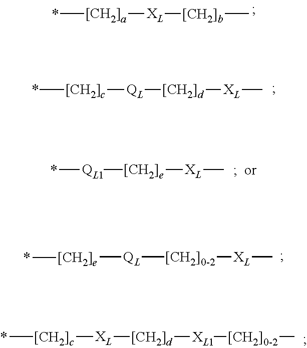

wherein:
   * denotes the point of attachment to the N atom in formula I;
   X_{L} or X_{L1} is absent or selected from —R_{L5}C=CR_{L6}—, ethynylene, —O—, —N(R_{LN})—, —C(O)—N(R_{LN})—, —N(R_{LN})—C(O)—, —S(O)₀₋₂—, —SO₂N(R_{LN})— or —N(R_{LN})SO₂—, wherein R_{L5} and R_{L6} are, at each occurrence, independently selected from hydrogen or methyl, and R_{LN} and R_{LN}1 are each independently selected from hydrogen or methyl;
   Q_{L} is selected from a (5-6C)cycloalkyl, a 5 to 7 membered heterocyclyl or a 5 or 6-membered heteroaryl ring;
   Q_{L}1 is selected from a (4-6C)cycloalkyl, a carbon-linked 5 to 7 membered heterocyclyl or a carbon-linked 5 or 6-membered heteroaryl ring;
   a is 1-2;
   b is 0-2;
   c is 1-2;
   d is 0-2; and
   e is 1-3;
(32) L is a linker group of the formula:

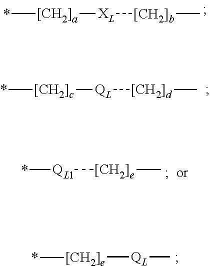

wherein:
* denotes the point of attachment to the N atom in formula I;
X_{L} is absent or selected from —R_{L5}C=CR_{L6}—, —O—, —N(R_{LN})—, —C(O)—N(R_{LN})— or —N(R_{LN})—C(O)—, wherein R_{L5} and R_{L6} are, at each occurrence, independently selected from hydrogen or methyl, and R_{LN} and R_{LN1} are each independently selected from hydrogen or methyl;
Q_{L} is selected from a (5-6C)cycloalkyl, a 5 to 7 membered heterocyclyl or a 5 or 6-membered heteroaryl ring;
Q_{L1} is selected from a (4-6C)cycloalkyl, a carbon-linked 5 to 7 membered heterocyclyl or a carbon-linked 5 or 6-membered heteroaryl ring;
a is 1-2;
b is 0-2;
c is 1-2;
d is 0-2; and
e is 1-3.
(33) L is a linker group of the formula:

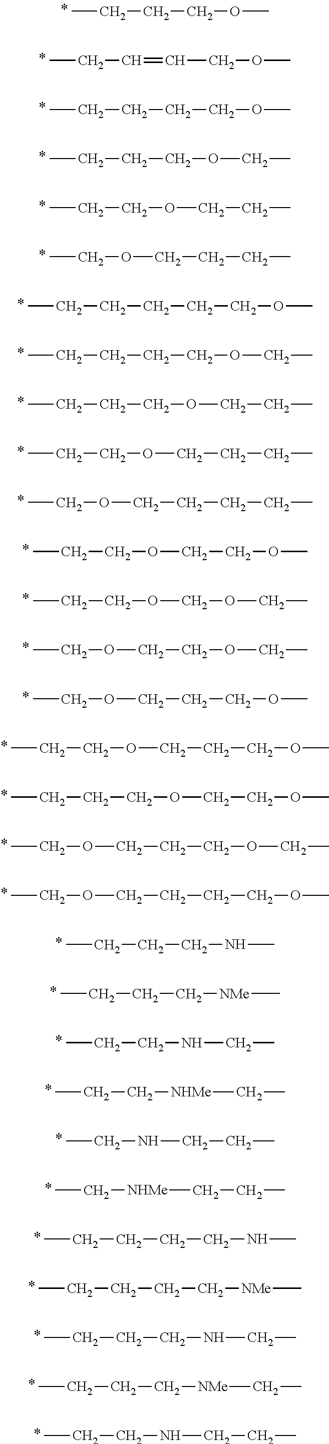

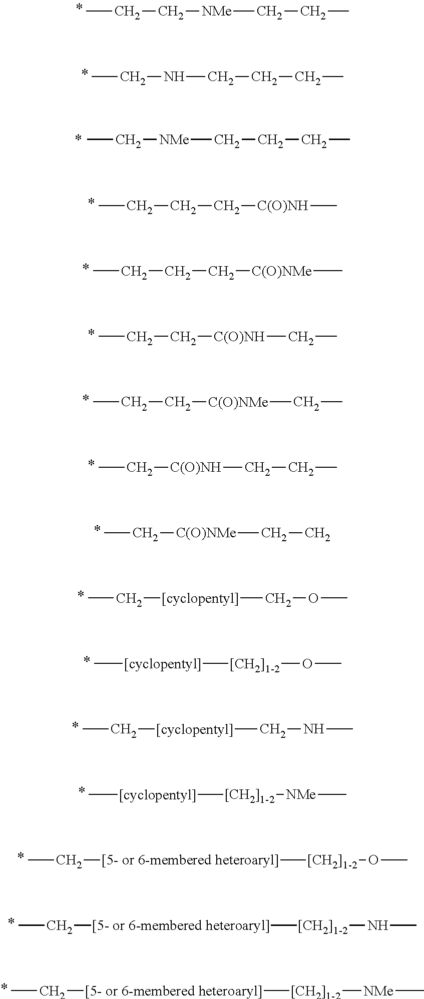

wherein:
   * denotes the point of attachment to the N atom in formula I.
(34) L is a linker group of the formula:

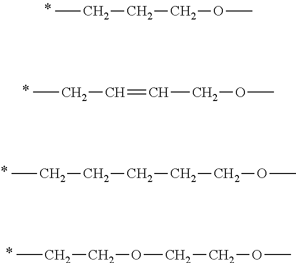

wherein:
   * denotes the point of attachment to the N atom in formula I.
(35) R_{N} is selected from hydrogen, (1-5C)alkyl, (3-6C)cycloalkyl, (3-6C)cycloalkyl(1-4C)alkyl, phenyl(1-4C)alkyl, carbon-linked 3- to 8-membered heterocyclyl, 3- to 8-membered heterocyclyl(1-4C)alkyl, 5- or 6-membered heteroaryl and [5- or 6-membered heteroaryl](1-4C)alkyl,
   wherein any alkyl, cycloalkyl, cycloalkylalkyl, phenylalkyl, heterocyclyl, heterocyclylalkyl, heteroaryl or heteroarylalkyl in R_{N} is optionally substituted with one or more R_{NA} groups as defined above or in any one of paragraphs (4) to (10) above;
(36) R_{N} is selected from hydrogen, (1-4C)alkyl, (3-6C)cycloalkyl, (3-6C)cycloalkyl(1-4C)alkyl, phenyl(1-4C)alkyl, carbon-linked 4- to 6-membered heterocyclyl, 4- to 6-membered heterocyclyl(1-4C)alkyl, 5- or 6-membered heteroaryl and [5- or 6-membered heteroaryl](1-4C)alkyl,
   wherein any alkyl, cycloalkyl, cycloalkylalkyl, phenylalkyl, heterocyclyl, heterocyclylalkyl, heteroaryl or heteroarylalkyl in R_{N} is optionally substituted with one or more R_{NA} groups as defined above or in any one of paragraphs (4) to (10) above;
(37) R_{N} is selected from (1-4C)alkyl, (3-6C)cycloalkyl, (3-6C)cycloalkyl(1-2C)alkyl, phenyl(1-2C)alkyl, carbon-linked 5- to 6-membered heterocyclyl, 5- to 6-membered heterocyclyl(1-2C)alkyl, 5- or 6-membered heteroaryl and [5- or 6-membered heteroaryl](1-2C)alkyl,
   wherein any alkyl, cycloalkyl, cycloalkylalkyl, phenylalkyl, heterocyclyl, heterocyclylalkyl, heteroaryl or heteroarylalkyl in R_{N} is optionally substituted with one or more R_{NA} groups as defined above or in any one of paragraphs (4) to (10) above;
(38) R_{N} is selected from hydrogen, (1-5C)alkyl, (3-6C)cycloalkyl, (3-6C)cycloalkyl(1-4C)alkyl, phenyl(1-4C)alkyl, carbon-linked 3- to 8-membered heterocyclyl, 3- to 8-membered heterocyclyl(1-4C)alkyl and [5- or 6-membered heteroaryl](1-4C)alkyl,
   wherein any alkyl, cycloalkyl, cycloalkylalkyl, phenylalkyl, heterocyclyl, heterocyclylalkyl or heteroarylalkyl in R_{N} is optionally substituted with one or more R_{NA} groups as defined above or in any one of paragraphs (4) to (10) above;
(39) R_{N} is selected from hydrogen, (1-4C)alkyl, (3-6C)cycloalkyl, (3-6C)cycloalkyl(1-4C)alkyl, phenyl(1-4C)alkyl, carbon-linked 4- to 6-membered heterocyclyl, 4- to 6-membered heterocyclyl(1-4C)alkyl and [5- or 6-membered heteroaryl](1-4C)alkyl,
   wherein any alkyl, cycloalkyl, cycloalkylalkyl, phenylalkyl, heterocyclyl, heterocyclylalkyl or heteroarylalkyl in R_{N} is optionally substituted with one or more R_{NA} groups as defined above or in any one of paragraphs (4) to (10) above;
(40) R_{N} is selected from (1-4C)alkyl, (3-6C)cycloalkyl, (3-6C)cycloalkyl(1-2C)alkyl, phenyl(1-2C)alkyl, carbon-linked 5- to 6-membered heterocyclyl, 5- to 6-membered heterocyclyl(1-2C)alkyl and [5- or 6-membered heteroaryl](1-2C)alkyl,
   wherein any alkyl, cycloalkyl, cycloalkylalkyl, phenylalkyl, heterocyclyl, heterocyclylalkyl or heteroarylalkyl in R_{N} is optionally substituted with one or more R_{NA} groups as defined above or in any one of paragraphs (4) to (10) above;
(41) R_{N} is selected from hydrogen, (1-5C)alkyl, (3-6C)cycloalkyl, (3-6C)cycloalkyl(1-4C)alkyl, carbon-linked 3- to 8-membered heterocyclyl, 3- to 8-membered heterocyclyl(1-4C)alkyl and [5- or 6-membered heteroaryl](1-4C)alkyl,
   wherein any alkyl, cycloalkyl, cycloalkylalkyl, heterocyclyl, heterocyclylalkyl or heteroarylalkyl in R_{N} is optionally substituted with one or more R_{NA} groups as defined above or in any one of paragraphs (4) to (10) above;
(42) R_{N} is selected from hydrogen, (1-4C)alkyl, (3-6C)cycloalkyl, (3-6C)cycloalkyl(1-4C)alkyl, carbon-linked 4- to 6-membered heterocyclyl, 4- to 6-membered heterocyclyl(1-4C)alkyl and [5- or 6-membered heteroaryl](1-4C)alkyl,
   wherein any alkyl, cycloalkyl, cycloalkylalkyl, heterocyclyl, heterocyclylalkyl or heteroarylalkyl in R_{N} is optionally substituted with one or more R_{NA} groups as defined above or in any one of paragraphs (4) to (10) above;
(43) R_{N} is selected from (1-4C)alkyl, (3-6C)cycloalkyl, (3-6C)cycloalkyl(1-2C)alkyl, carbon-linked 5- to 6-membered heterocyclyl, 5- to 6-membered heterocyclyl(1-2C)alkyl and [5- or 6-membered heteroaryl](1-2C)alkyl,
   wherein any alkyl, cycloalkyl, cycloalkylalkyl, heterocyclyl, heterocyclylalkyl or heteroarylalkyl in R_{N} is optionally substituted with one or more R_{NA} groups as defined above or in any one of paragraphs (4) to (10) above;
(44) R_{N} is selected from hydrogen, (1-5C)alkyl, (3-6C)cycloalkyl, (3-6C)cycloalkyl(1-4C)alkyl, carbon-linked 3- to 8-membered heterocyclyl and 3- to 8-membered heterocyclyl(1-4C)alkyl,
   wherein any alkyl, cycloalkyl, cycloalkylalkyl, heterocyclyl or heterocyclylalkyl in R_{N} is optionally substituted with one or more R_{NA} groups as defined above or in any one of paragraphs (4) to (10) above;
(45) R_{N} is selected from hydrogen, (1-4C)alkyl, (3-6C)cycloalkyl, (3-6C)cycloalkyl(1-4C)alkyl, carbon-linked 4- to 6-membered heterocyclyl and 4- to 6-membered heterocyclyl(1-4C)alkyl,
   wherein any alkyl, cycloalkyl, cycloalkylalkyl, heterocyclyl or heterocyclylalkyl in R_{N} is optionally substituted with one or more R_{NA} groups as defined above or in any one of paragraphs (4) to (10) above;
(46) R_{N} is selected from (1-4C)alkyl, (3-6C)cycloalkyl, (3-6C)cycloalkyl(1-2C)alkyl, carbon-linked 5- to 6-membered heterocyclyl and 5- to 6-membered heterocyclyl(1-2C)alkyl,
   wherein any alkyl, cycloalkyl, cycloalkylalkyl, heterocyclyl or heterocyclylalkyl in R_{N} is optionally substituted with one or more R_{NA} groups as defined above or in any one of paragraphs (4) to (10) above;
(47) R_{N} is selected from (3-6C)cycloalkyl, (3-6C)cycloalkyl(1-2C)alkyl, carbon-linked 5- to 6-membered heterocyclyl, 5- to 6-membered heterocyclyl(1-2C)alkyl or 5- or 6-membered heteroaryl-(1-2C)alkyl,
   wherein any cycloalkyl, cycloalkylalkyl, heterocyclyl, heterocyclylalkyl or heteroarylalkyl in R_{N} is optionally substituted with one or more R_{NA} groups as defined above or in any one of paragraphs (4) to (10) above;
(48) R_{N} is selected from (4-6C)cycloalkyl, (4-6C)cycloalkyl-CH₂—, 5- to 6-membered heterocyclyl-CH₂— or 5- or 6-membered heteroaryl-CH₂—,
   wherein any cycloalkyl, cycloalkyl-CH₂—, heterocyclyl-CH₂— or heteroaryl-CH₂-in R_{N} is optionally substituted with one or more R_{NA} groups as defined above or in any one of paragraphs (4) to (10) above;
(49) R_{N} is selected from (4-6C)cycloalkyl, (4-6C)cycloalkyl-CH₂—, 5- to 6-membered heterocyclyl-CH₂— or 5- or 6-membered heteroaryl-CH₂—,
   wherein a heteroaryl is optionally substituted with (1-3C)alkyl;
(50) R_{N} is selected from:

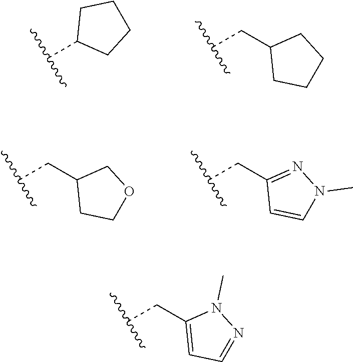

(51) Q₁ is selected from hydrogen or:
   (i) a group of the formula:

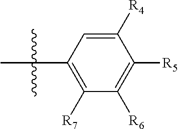

wherein:
R₄, R₆ and R₇ are each independently selected from hydrogen, halo, cyano, or a group of the formula:

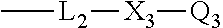

wherein:
L₂ is absent or (1-3C)alkylene;
X₃ is absent or is selected from the group consisting of —O—, —NR₄ₐ—, —C(O)—O—, —O—C(O)—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclyl-alkyl, heteroaryl or heteroaryl-alkyl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl; and wherein any alkyl, alkoxy, cycloalkyl, cycloalkyl-alkyl group present in a R_{4c} group, or any alkyl group present in a R_{4d} or R₄ₑ group, is optionally further substituted by one or more substituents independently selected from hydroxy, cyano, halogen, R_{4f}, —OR_{4f}, —NR_{4f}R_{4g}—C(O)OR_{4f}, —OC(O)R_{4f}, —C(O)NR_{4f}R_{4g}, —NR₄₉C(O)R_{4f}, —S(O)₀₋₂—R_{4f} and —C(O)—R_{4f}, wherein R_{4f} and R₄₉ are both independently selected from hydrogen or (1-2C)alkyl;
R₅ is:
   a) hydrogen, halo, cyano; or
   b) a group of the formula:

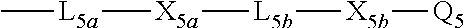

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

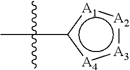

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

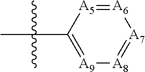

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

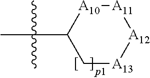

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

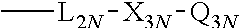

wherein:
L_{2N} is absent or (1-3C)alkylene;
X_{3N} is absent or is selected from the group consisting of:

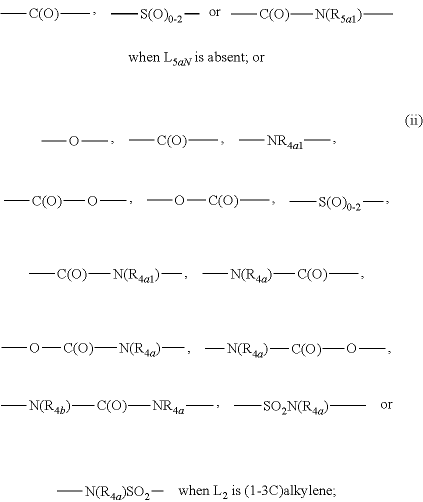

wherein R₄ₐ and R_{4b} are as defined above;
Q_{3N} is selected from the group consisting of hydrogen, (1-6C)alkyl, (3-6C)cycloalkyl, (3-6C)cycloalkyl(1-2C)alkyl, heterocyclyl, heterocyclyl(1-2C)alkyl, heteroaryl and heteroaryl(1-2C)alkyl, wherein any alkyl, cycloalkyl, cycloalkylalkyl, heterocyclyl, heterocyclylalkyl, heteroaryl or heteroarylalkyl in Q₃ is optionally substituted with one or more R_{4c} groups; and
each R_{4c} group present is as defined above;
R_{5N} is:
   a) hydrogen;
   b) a group of the formula:

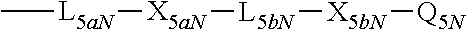

wherein:
La_{N} is absent or (1-3C)alkylene;
X_{5aN} is absent or is selected from the group consisting of:

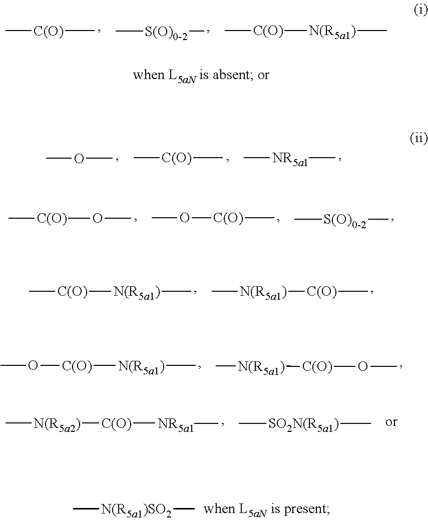

where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

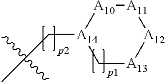

wherein:
p1, A₁₀, A₁₁, A₁₂, and A₁₃ are as defined above;
p2 is 1 to 5;
A₁₄ is C or N;
and with the proviso that one of A₁₀, A₁₁, A₁₂ and A₁₃ can be N;
or
R_{N} and R₄ are linked to form a linker group L as defined herein;
(52) Q₁ is selected from hydrogen or:
   (i) a group of the formula:

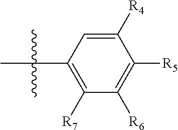

wherein:
R₄, R₆ and R₇ are each independently selected from hydrogen, or a group of the formula:

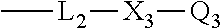

wherein:
L₂ is absent or (1-3C)alkylene;
X₃ is absent or is selected from the group consisting of —O—, —NR₄ₐ—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-8C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl, or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
and wherein any alkyl, alkoxy, cycloalkyl, cycloalkyl-alkyl, phenyl, heteroaryl or heterocyclyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f},
wherein R_{4f} and R₄₉ are both independently selected from hydrogen and (1-2C)alkyl;
R₅ is:
   a) hydrogen, halo, cyano; or
   b) a group of the formula:

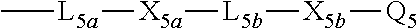

wherein:
L₅ₐ is absent or (1-3C)alkylene;
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

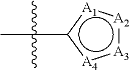

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

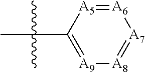

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

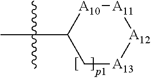

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

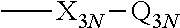

wherein:
X_{3N} is absent or is selected from the group consisting of: —C(O)—, —S(O)₀₋₂— or —C(O)—N(R₄ₐ)—;
   wherein R₄ₐ is as defined above;
Q_{3N} is selected from the group consisting of hydrogen, (1-4C)alkyl, (3-6C)cycloalkyl, (3-6C)cycloalkyl(1-2C)alkyl, heterocyclyl, heteroaryl and, wherein any alkyl, cycloalkyl, cycloalkylalkyl, heterocyclyl or heteroaryl in Q₃ is optionally substituted with one or more R_{4c} groups; and
each R_{4c} group present is as defined above;
R_{5N} is:
   a) hydrogen;
   b) a group of the formula:

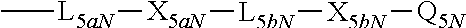

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, S(O)₀₋₂—, —C(O)—N(R₅ₐ₁)—, —N(R₅ₐ₁)—C(O)—, O—C(O)—N(R₅ₐ₁)—, —N(R₅ₐ₁)—C(O)—O—, N(R₅ₐ₂)—C(O)—NR₅ₐ₁—, —SO₂N(R₅ₐ₁)— or N(R₅ₐ₁)SO₂— when L_{5aN} is present;

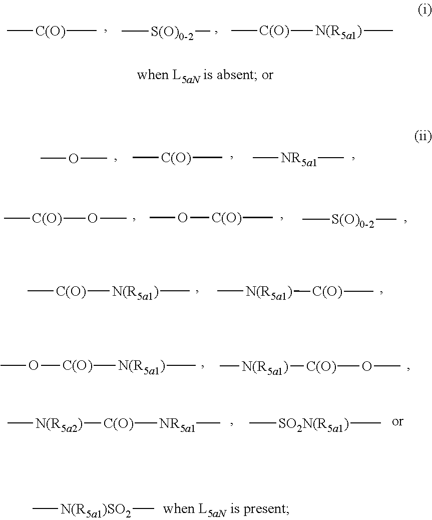

where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

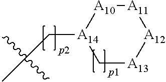

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or
R_{N} and R₄ are linked to form a linker group L as defined herein;
(53) Q₁ is selected from hydrogen or:
   (i) a group of the formula:

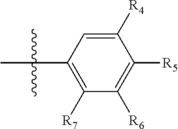

wherein:
R₄, R₆ and R₇ are each independently selected from hydrogen, or a group of the formula:

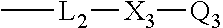

wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-2C)alkyl; and wherein any alkyl, alkoxy, cycloalkyl, cycloalkyl-alkyl, phenyl, heteroaryl or heterocyclyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f}, wherein R_{4f} and R₄₉ are both independently selected from hydrogen and (1-2C)alkyl;
R₅ is:
   a) hydrogen, halo, cyano; or
   b) a group of the formula:

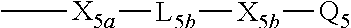

wherein:
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

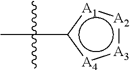

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

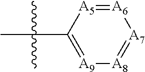

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

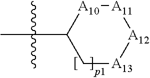

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
   a) hydrogen;
   b) a group of the formula:

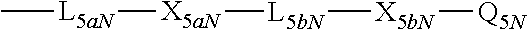

wherein:
L_{5aN} is absent or (1-3C)alkylene;
X_{5aN} is absent or is selected from the group consisting of:

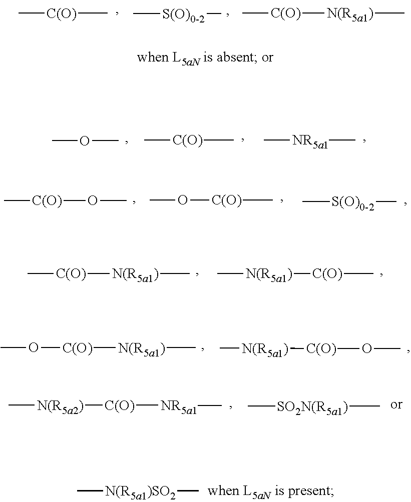

where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

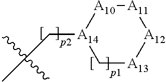

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or
R_{N} and R₄ are linked to form a linker group L as defined herein;
(54) Q₁ is selected from hydrogen or:
   (i) a group of the formula:

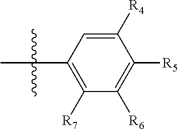

wherein:
R₄, R₆ and R₇ are each independently selected from hydrogen, or a group of the formula:

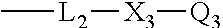

wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d}, wherein R_{4d} and R₄ₑ are each independently hydrogen or methyl;
and wherein any alkyl, alkoxy, cycloalkyl, cycloalkyl-alkyl, phenyl, heteroaryl or heterocyclyl group present in a R_{4c} group, or any methyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f},
wherein R_{4f} and R₄₉ are both independently selected from hydrogen and (1-2C)alkyl;
R₅ is:
   a) hydrogen, halo, cyano; or
   b) a group of the formula:

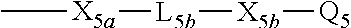

wherein:
X₅ₐ is absent or is selected from the group consisting of —O—, —C(O)—, —NR₅ₐ₁— or —N(R₅ₐ)—C(O)—, where R₅ₐ₁ is independently selected from the group consisting of hydrogen or methyl;
L_{5b} is absent or (1-4C)alkylene;
X_{5b} is absent or is selected from the group consisting of —O—, —C(O)— or —NR_{5b1}—, where R_{5b}, and R_{5b2} are independently selected from the group consisting of hydrogen or (1-2C)alkyl;
Q₅ is selected from the group consisting of hydrogen, (1-6C)alkyl or 3 to 8-membered heterocyclyl and wherein Q₅ is optionally substituted with one or more R_{5c} groups;
and each R_{5c} group present is independently selected from the group consisting of hydroxy, cyano, halogen, methyl, methoxy
(ii) a group of the formula:

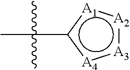

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

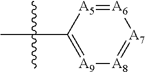

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

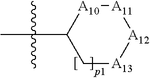

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is
   a) hydrogen;
   b) a group of the formula:

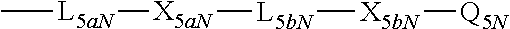

wherein:
L_{5aN} is absent or (1-3C)alkylene;
X_{5aN} is absent or is selected from the group consisting of:

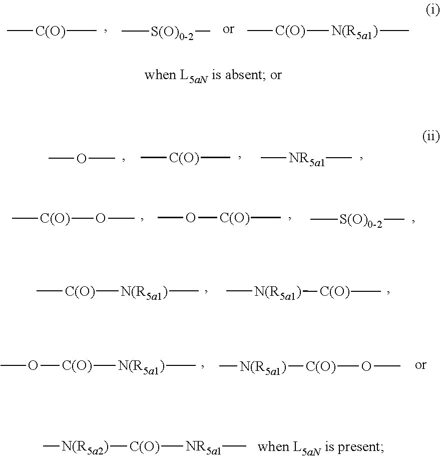

where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—,
where R_{5b1} and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

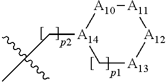

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or
R_{N} and R₄ are linked to form a linker group L as defined herein;
(55) Q₁ is selected from hydrogen or:
   (i) a group of the formula:

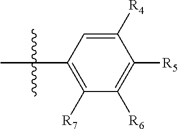

wherein:
two of R₄, R₆ and R₇ are hydrogen, and the other is hydrogen or a group of the formula:

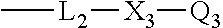

wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d}, wherein R_{4d} and R₄ₑ are each independently hydrogen or methyl;
and wherein any methyl, methoxy or cycloalkyl, group present in a R_{4c} group, or any methyl group present in a R_{4d} or R₄ₑ group, is optionally further substituted by one or more substituents independently selected from hydroxy, cyano, halogen, —OR_{4f}, —NR₄ᵣR_{4g}, —S(O)₀₋₂—R₄ᵣ and —C(O)—R₄ᵣ, wherein R₄ᵣ and R₄₉ are both independently selected from hydrogen and (1-2C)alkyl;
R₅ is hydrogen or a group of the formula:

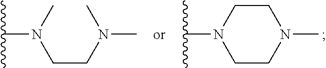

and
(ii) a group of the formula:

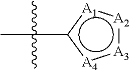

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R₅ₑ groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

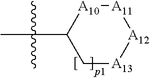

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
   a) hydrogen;
   b) a group of the formula:

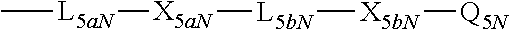

wherein:
L_{5aN} is absent or (1-3C)alkylene;
X_{5aN} is absent or is selected from the group consisting of:
(i) —C(O)—, —S(O)₀₋₂— or —C(O)—N(R₅ₐ₁)— when L_{5aN} iS absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(iv) a group of the formula:

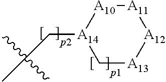

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or
R_{N} and R₄ are linked to form a linker group L as defined herein.

Suitably, in any of the definitions of formula I set out herein, a heteroaryl is a 5- or 6-membered heteroaryl ring comprising one, two or three heteroatoms selected from N, O or S. More suitably, in any of the definitions of formula I set out herein, a heteroaryl is a 5- or 6-membered heteroaryl ring comprising one or two N atoms.

Suitably, in any of the definitions of formula I set out herein, a heterocyclyl group is a 4-, 5-, 6- or 7-membered heterocyclyl ring comprising one, two or three heteroatoms selected from N, O or S. Most suitably, a heterocyclyl group is a 4-, 5- or 6-membered ring comprising one or two heteroatoms selected from N, O or S [e.g. morpholinyl (e.g. 4-morpholinyl), piperidinyl, piperazinyl or pyrrolidinyl].

Suitably, in any of the definitions of formula I set out herein, R_{N} is as defined in formula I above or is as defined in either of paragraphs (1), (2) or (3) above. In a particular group of compounds of the invention, R_{N} is as defined in paragraph (1) above. In another particular group of compounds of the invention, R_{N} is as defined in paragraph (2) above. In another particular group of compounds of the invention, R_{N} is as defined in paragraph (3) above.

In a particular group of compounds of the invention, R_{N} is as defined any one of paragraphs (35) to (50) above. In a particular group of compounds of the invention, R_{N} is as defined in paragraph (35) above. In another particular group of compounds of the invention, R_{N} is as defined in paragraph (36) above. In another particular group of compounds of the invention, R_{N} is as defined in paragraph (37) above. In another particular group of compounds of the invention, R_{N} is as defined in paragraph (38) above. In another particular group of compounds of the invention, R_{N} is as defined in paragraph (39) above. In another particular group of compounds of the invention, R_{N} is as defined in paragraph (40) above. In another particular group of compounds of the invention, R_{N} is as defined in paragraph (41) above. In another particular group of compounds of the invention, R_{N} is as defined in paragraph (42) above. In another particular group of compounds of the invention, R_{N} is as defined in paragraph (43) above. In another particular group of compounds of the invention, R_{N} is as defined in paragraph (44) above. In another particular group of compounds of the invention, R_{N} is as defined in paragraph (45) above. In another particular group of compounds of the invention, R_{N} is as defined in paragraph (46) above. In another particular group of compounds of the invention, R_{N} is as defined in paragraph (47) above. In another particular group of compounds of the invention, R_{N} is as defined in paragraph (48) above. In another particular group of compounds of the invention, R_{N} is as defined in paragraph (49) above. In another particular group of compounds of the invention, R_{N} is as defined in paragraph (50) above.

Suitably, in any of the definitions of formula I set out herein, R_{NA} is as defined in formula I above or is as defined in either of paragraphs (4), (5), (6), (7), (8), (9) or (10) above. In a particular group of compounds of the invention, R_{NA} is as defined in paragraph (4) above. In another particular group of compounds of the invention, R_{NA} is as defined in paragraph (5) above. In another particular group of compounds of the invention, R_{NA} is as defined in paragraph (6) above. In another particular group of compounds of the invention, R_{NA} is as defined in paragraph (7) above. In another particular group of compounds of the invention, R_{NA} is as defined in paragraph (8) above. In another particular group of compounds of the invention, R_{NA} is as defined in paragraph (9) above. In another particular group of compounds of the invention, R_{NA} is as defined in paragraph (10) above.

In a particular group of compounds of the invention, R_{N} is as defined in paragraph (1) above and R_{NA} is as defined in formula I above or is as defined any one of paragraphs (4), (5), (6), (7), (8), (9) or (10) above.

In a particular group of compounds of the invention, R_{N} is as defined in paragraph (2) above and R_{NA} is as defined in formula I above or is as defined any one of paragraphs (4), (5), (6), (7), (8), (9) or (10) above.

In a particular group of compounds of the invention, R_{N} is as defined in paragraph (3) above and R_{NA} is as defined in formula I above or is as defined any one of paragraphs (4), (5), (6), (7), (8), (9) or (10) above.

Suitably, in any of the definitions of formula I set out herein, R₁ is as defined in formula I above or is as defined in any one of paragraphs (11), (12) or (13) above. In a particular group of compounds of the invention, R₁ is as defined in paragraph (11) above. In another particular group of compounds of the invention, R₁ is as defined in paragraph (12) above. In another particular group of compounds of the invention, R₁ is as defined in paragraph (13) above.

Suitably, in any of the definitions of formula I set out herein, V₁ is as defined in formula I above or is as defined in any one of paragraphs (14) or (15) above. In a particular group of compounds of the invention, V₁ is as defined in paragraph (14) above. In another particular group of compounds of the invention, V₁ is as defined in paragraph (15) above.

Suitably, in any of the definitions of formula I set out herein, R₂ is as defined in formula I above or is as defined in any one of paragraphs (16), (17), (18), (19), (20), or (21) above. In a particular group of compounds of the invention, R₂ is as defined in paragraph (16) above. In another particular group of compounds of the invention, R₂ is as defined in paragraph (17) above. In another particular group of compounds of the invention, R₂ is as defined in paragraph (18) above. In another particular group of compounds of the invention, R₂ is as defined in paragraph (19) above. In another particular group of compounds of the invention, R₂ is as defined in paragraph (20) above. In another particular group of compounds of the invention, R₂ is as defined in paragraph (21) above.

Suitably, in any of the definitions of formula I set out herein, Q₁ is as defined in formula I above or is as defined in any one of paragraphs (22), (23), (24) or (25) above. In a particular group of compounds of the invention, Q₁ is as defined in paragraph (22) above. In another particular group of compounds of the invention, Q₁ is as defined in paragraph (23) above. In another particular group of compounds of the invention, Q₁ is as defined in paragraph (24) above. In another particular group of compounds of the invention, Q₁ is as defined in paragraph (25) above.

Suitably, in any of the definitions of formula I set out herein, Q₁ is also as defined in formula I above or is as defined in any one of paragraphs (51), (52), (53), (54) or (55) above. In a particular group of compounds of the invention, Q₁ is as defined in paragraph (51) above. In another particular group of compounds of the invention, Q₁ is as defined in paragraph (52) above. In another particular group of compounds of the invention, Q₁ is as defined in paragraph (53) above. In another particular group of compounds of the invention, Q₁ is as defined in paragraph (54) above. In a particular group of compounds of the invention, Q₁ is as defined in paragraph (55) above.

Suitably, in any of the definitions of formula I set out herein, L is as defined in formula I above or is as defined in any one of paragraphs (27), (28), (29), (30) or (31) above. In a particular group of compounds of the invention, L is as defined in paragraph (27) above. In another particular group of compounds of the invention, L is as defined in paragraph (28) above. In another particular group of compounds of the invention, L is as defined in paragraph (29) above. In another particular group of compounds of the invention, L is as defined in paragraph (30) above. In another particular group of compounds of the invention, L is as defined in paragraph (31) above.

In a particular group of compounds of formula I defined herein:
R₁ and V₁ are both as defined in formula I above;
R_{N} is as defined in paragraph (1) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (2) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (35) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (36) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (37) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (38) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (39) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (40) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (41) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (42) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (43) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (44) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (45) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R_{N} is as defined in paragraph (46) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (47) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (48) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (49) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R_{N} is as defined in paragraph (50) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula I defined herein:
R₁ and V₁ are both as defined in formula I above;
R_{N} is as defined in paragraph (1) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (2) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R_{N} is as defined in paragraph (35) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (36) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (37) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (38) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R_{N} is as defined in paragraph (39) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (40) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (41) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (42) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R_{N} is as defined in paragraph (43) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (44) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (45) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (46) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R_{N} is as defined in paragraph (47) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (48) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (49) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R₁ and V₁ are both as defined in formula I above;
R_{N} is as defined in paragraph (50) above and R_{NA} is as defined in formula I above or is as defined any one of paragraphs (4), (5), (6), (7), (8), (9) or (10) above;
R₂ is as defined in paragraph (17) above;
Q₁ is as defined in paragraph (23) above; and
L is as defined in paragraph (29) above.

In a particular group of compounds of formula I defined herein:
R_{N} is as defined in paragraph (1) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (2) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (35) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R_{N} is as defined in paragraph (36) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (37) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (38) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (39) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R_{N} is as defined in paragraph (40) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (41) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (42) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (43) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R_{N} is as defined in paragraph (44) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (45) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (46) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (47) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R_{N} is as defined in paragraph (48) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (49) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (50) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula I defined herein:
R₁ and V₁ are both as defined in formula I above;
R_{N} is as defined in paragraph (1) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R_{N} is as defined in paragraph (2) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (35) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (36) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R_{N} is as defined in paragraph (37) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (38) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (39) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (40) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R_{N} is as defined in paragraph (41) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (42) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (43) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (44) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R_{N} is as defined in paragraph (45) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (46) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (47) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (48) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R_{N} is as defined in paragraph (49) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (50) above and R_{NA} is as defined in formula I above or is as defined any one of paragraphs (4), (5), (6), (7), (8), (9) or (10) above;
R₂ is as defined in paragraph (19) above;
Q₁ is as defined in paragraph (25) above; and
L is as defined in paragraph (31) above.

In a particular group of compounds of formula I defined herein:
R₁ and V₁ are both as defined in formula I above;
R_{N} is as defined in paragraph (1) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (2) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R_{N} is as defined in paragraph (3) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (35) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (36) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (37) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R_{N} is as defined in paragraph (38) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (39) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (40) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (41) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R_{N} is as defined in paragraph (42) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (43) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (44) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (45) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R_{N} is as defined in paragraph (46) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (47) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (48) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (49) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R_{N} is as defined in paragraph (50) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula I defined herein:
R₁ and V₁ are both as defined in formula I above;
R_{N} is as defined in paragraph (1) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (2) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R_{N} is as defined in paragraph (35) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (36) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (37) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (38) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R_{N} is as defined in paragraph (39) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (40) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (41) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (42) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R_{N} is as defined in paragraph (43) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (44) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (45) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (46) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R_{N} is as defined in paragraph (47) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (48) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (49) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above.

In a particular group of compounds of formula I defined herein:
R₁ and V₁ are both as defined in formula I above;
R_{N} is as defined in paragraph (50) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (33) above. In a particular group of compounds of formula I defined herein:
R_{N} is as defined in paragraph (1) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (2) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (35) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R_{N} is as defined in paragraph (36) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (37) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (38) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:

R₁ and V₁ are both as defined in formula I above;
R_{N} is as defined in paragraph (39) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R_{N} is as defined in paragraph (40) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (41) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (42) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (43) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R_{N} is as defined in paragraph (44) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (45) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (46) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (47) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R_{N} is as defined in paragraph (48) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (49) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
R₁ and V₁ are both as defined in formula I above;
R_{N} is as defined in paragraph (50) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (1) above and R_{NA} is as defined in formula I above or is as defined any one of paragraphs (4), (5), (6), (7), (8), (9) or (10) above;
R₁ is as defined in paragraph (11) above;
R₂ is as defined in paragraph (16) above;
Q₁ is as defined in paragraph (22) above; and
L is as defined in paragraph (28) above.

In a particular group of compounds of formula I defined herein:
V₁ is as defined in formula I above;
R_{N} is as defined in paragraph (35) above and R_{NA} is as defined in formula I above or is as defined any one of paragraphs (4), (5), (6), (7), (8), (9) or (10) above;
R₁ is as defined in paragraph (11) above;
R₂ is as defined in paragraph (16) above;
Q₁ is as defined in paragraph (22) above; and
L is as defined in paragraph (28) above.

In a particular group of compounds of formula I defined herein:
V₁ is as defined in formula I above;
R_{N} is as defined in paragraph (38) above and R_{NA} is as defined in formula I above or is as defined any one of paragraphs (4), (5), (6), (7), (8), (9) or (10) above;
R₁ is as defined in paragraph (11) above;
R₂ is as defined in paragraph (16) above;
Q₁ is as defined in paragraph (22) above; and
L is as defined in paragraph (28) above.

In a particular group of compounds of formula I defined herein:
V₁ is as defined in formula I above;
R_{N} is as defined in paragraph (41) above and R_{NA} is as defined in formula I above or is as defined any one of paragraphs (4), (5), (6), (7), (8), (9) or (10) above;
R₁ is as defined in paragraph (11) above;
R₂ is as defined in paragraph (16) above;
Q₁ is as defined in paragraph (22) above; and
L is as defined in paragraph (28) above.

In a particular group of compounds of formula I defined herein:
V₁ is as defined in formula I above;
R_{N} is as defined in paragraph (44) above and R_{NA} is as defined in formula I above or is as defined any one of paragraphs (4), (5), (6), (7), (8), (9) or (10) above;
R₁ is as defined in paragraph (11) above;
R₂ is as defined in paragraph (16) above;
Q₁ is as defined in paragraph (22) above; and
L is as defined in paragraph (28) above.

In a particular group of compounds of formula I defined herein:
V₁ is as defined in formula I above;
R_{N} is as defined in paragraph (2) above and R_{NA} is as defined in formula I above or is as defined any one of paragraphs (4), (5), (6), (7), (8), (9) or (10) above;
R₁ is as defined in paragraph (11) above;
R₂ is as defined in paragraph (18) above;
Q₁ is as defined in paragraph (24) above; and
L is as defined in paragraph (30) above.

In a particular group of compounds of formula I defined herein:
V₁ is as defined in formula I above;
R_{N} is as defined in paragraph (36) above and R_{NA} is as defined in formula I above or is as defined any one of paragraphs (4), (5), (6), (7), (8), (9) or (10) above;
R₁ is as defined in paragraph (11) above;
R₂ is as defined in paragraph (18) above;
Q₁ is as defined in paragraph (24) above; and
L is as defined in paragraph (30) above.

In a particular group of compounds of formula I defined herein:
V₁ is as defined in formula I above;
R_{N} is as defined in paragraph (39) above and R_{NA} is as defined in formula I above or is as defined any one of paragraphs (4), (5), (6), (7), (8), (9) or (10) above;
R₁ is as defined in paragraph (11) above;
R₂ is as defined in paragraph (18) above;
Q₁ is as defined in paragraph (24) above; and
L is as defined in paragraph (30) above.

In a particular group of compounds of formula I defined herein:
V₁ is as defined in formula I above;
R_{N} is as defined in paragraph (42) above and R_{NA} is as defined in formula I above or is as defined any one of paragraphs (4), (5), (6), (7), (8), (9) or (10) above;
R₁ is as defined in paragraph (11) above;
R₂ is as defined in paragraph (18) above;
Q₁ is as defined in paragraph (24) above; and
L is as defined in paragraph (30) above.

In a particular group of compounds of formula I defined herein:
V₁ is as defined in formula I above;
R_{N} is as defined in paragraph (45) above and R_{NA} is as defined in formula I above or is as defined any one of paragraphs (4), (5), (6), (7), (8), (9) or (10) above;
R₁ is as defined in paragraph (11) above;
R₂ is as defined in paragraph (18) above;
Q₁ is as defined in paragraph (24) above; and
L is as defined in paragraph (30) above.

In a particular group of compounds of formula I defined herein:
V₁ is as defined in formula I above;
R_{N} is as defined in paragraph (3) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (37) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (40) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (43) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (46) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (47) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (48) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (49) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (50) above and R_{NA} is as defined in formula I above or is as defined any one of paragraphs (4), (5), (6), (7), (8), (9) or (10) above;
R₁ is as defined in paragraph (11) above;
R₂ is as defined in paragraph (20) above;
Q₁ is as defined in paragraph (25) above; and
L is as defined in paragraph (32) above.

In a particular group of compounds of formula I defined herein:
V₁ is as defined in formula I above;
R_{N} is as defined in paragraph (3) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (35) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (36) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (37) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (38) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (39) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (40) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (41) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (42) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (43) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (44) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (45) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (46) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (47) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (48) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (49) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a particular group of compounds of formula I defined herein:
V₁ is as defined in formula I above;
R_{N} is as defined in paragraph (50) above and R_{NA} is as defined in formula I above or is as defined any one of paragraphs (4), (5), (6), (7), (8), (9) or (10) above;
R₁ is as defined in paragraph (11) above;
R₂ is as defined in paragraph (21) above;
Q₁ is as defined in paragraph (25) above; and
L is as defined in paragraph (34) above.

In a further group of compounds of formula I defined herein:
V₁, R_{N}, R_{NA}, R₁, R₂ and L are as defined in any of the preceding paragraphs; and
Q₁ is as defined in paragraph (51) above.

In a further group of compounds of formula I defined herein:
V₁, R_{N}, R_{NA}, R₁, R₂ and L are as defined in any of the preceding paragraphs; and
Q₁ is as defined in paragraph (52) above.

In a further group of compounds of formula I defined herein:
V₁, R_{N}, R_{NA}, R₁. R₂ and L are as defined in any of the preceding paragraphs; and
Q₁ is as defined in paragraph (53) above.

In a further group of compounds of formula I defined herein:
V₁, R_{N}, R_{NA}, R₁, R₂ and L are as defined in any of the preceding paragraphs; and
Q₁ is as defined in paragraph (54) above.

In a further group of compounds of formula I defined herein:
V₁, R_{N}, R_{NA}, R₁, R₂ and L are as defined in any of the preceding paragraphs; and
Q₁ is as defined in paragraph (55) above.

In a particular group of compounds of the invention, the compound is a compound of formula I defined herein in which R₁ is ethynyl, i.e. the compounds have the formula Ia shown below, or a pharmaceutically acceptable salt thereof:

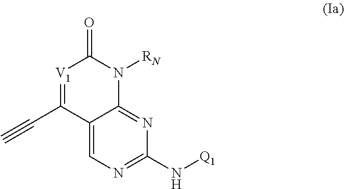

wherein R_{N}, V₁ and Q₁ each have any one of the definitions set out herein.

In a particular group of compounds of formula Ia:
V₁ is as defined in formula I above;
R_{N} is as defined in paragraph (1) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ia:
V₁ is as defined in formula I above;
R_{N} is as defined in paragraph (35) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ia:
V₁ is as defined in formula I above;
R_{N} is as defined in paragraph (38) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ia:
V₁ is as defined in formula I above;
R_{N} is as defined in paragraph (41) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ia:
V₁ is as defined in formula I above;
R_{N} is as defined in paragraph (44) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ia:
V₁ is as defined in formula I above;
R_{N} is as defined in paragraph (2) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ia:
V₁ is as defined in formula I above;
R_{N} is as defined in paragraph (36) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ia:
V₁ is as defined in formula I above;
R_{N} is as defined in paragraph (39) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ia:
V₁ is as defined in formula I above;
R_{N} is as defined in paragraph (42) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ia:
V₁ is as defined in formula I above;
R_{N} is as defined in paragraph (45) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ia:
V₁ is as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ia:
V₁ is as defined in formula I above;
R_{N} is as defined in paragraph (37) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ia:
V₁ is as defined in formula I above;
R_{N} is as defined in paragraph (40) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ia:
V₁ is as defined in formula I above;
R_{N} is as defined in paragraph (43) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ia:
V₁ is as defined in formula I above;
R_{N} is as defined in paragraph (46) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ia:
V₁ is as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ia:
V₁ is as defined in formula I above;
R_{N} is as defined in paragraph (47) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ia:
V₁ is as defined in formula I above;
R_{N} is as defined in paragraph (48) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ia:
V₁ is as defined in formula I above;
R_{N} is as defined in paragraph (49) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ia:
V₁ is as defined in formula I above;
R_{N} is as defined in paragraph (50) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a further group of compounds of formula Ia defined herein:
V₁, R_{N}, R_{NA}, R₂ and L are as defined in any of the preceding paragraphs; and
Q₁ is as defined in paragraph (51) above.

In a further group of compounds of formula Ia defined herein:
V₁, R_{N}, R_{NA}, R₂ and L are as defined in any of the preceding paragraphs; and
Q₁ is as defined in paragraph (52) above.

In a further group of compounds of formula Ia defined herein:
V₁, R_{N}, R_{NA}, R₂ and L are as defined in any of the preceding paragraphs; and
Q₁ is as defined in paragraph (53) above.

In a further group of compounds of formula Ia defined herein:
V₁, R_{N}, R_{NA}, R₂ and L are as defined in any of the preceding paragraphs; and
Q₁ is as defined in paragraph (54) above.

In a further group of compounds of formula Ia defined herein:
V₁, R_{N}, R_{NA}, R₂ and L are as defined in any of the preceding paragraphs; and
Q₁ is as defined in paragraph (55) above.

In a particular group of compounds of the invention, the compound is a compound of formula I defined herein in which R₁ is ethenyl, i.e. the compounds have the formula Ib shown below, or a pharmaceutically acceptable salt thereof:

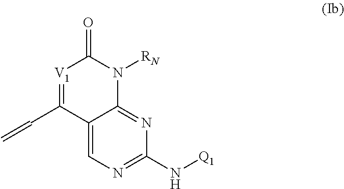

wherein R_{N}, V₁ and Q₁ each have any one of the definitions set out herein.

In a particular group of compounds of formula Ib:
V₁ is as defined in formula I above;
R_{N} is as defined in paragraph (1) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ib:
V₁ is as defined in formula I above;
R_{N} is as defined in paragraph (35) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ib:
V₁ is as defined in formula I above;
R_{N} is as defined in paragraph (38) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ib:
V₁ is as defined in formula I above;
R_{N} is as defined in paragraph (41) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ib:
V₁ is as defined in formula I above;
R_{N} is as defined in paragraph (44) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ib:
V₁ is as defined in formula I above;
R_{N} is as defined in paragraph (2) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ib:
V₁ is as defined in formula I above;
R_{N} is as defined in paragraph (36) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ib:
V₁ is as defined in formula I above;
R_{N} is as defined in paragraph (39) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ib:
V₁ is as defined in formula I above;
R_{N} is as defined in paragraph (42) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ib:
V₁ is as defined in formula I above;
R_{N} is as defined in paragraph (45) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ib:
V₁ is as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ib:
V₁ is as defined in formula I above;
R_{N} is as defined in paragraph (37) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ib:
V₁ is as defined in formula I above;
R_{N} is as defined in paragraph (40) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ib:
V₁ is as defined in formula I above;
R_{N} is as defined in paragraph (43) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ib:
V₁ is as defined in formula I above;
R_{N} is as defined in paragraph (46) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ib:
V₁ is as defined in formula I above;
R_{N} is as defined in paragraph (3) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ib:
V₁ is as defined in formula I above;
R_{N} is as defined in paragraph (47) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ib:
V₁ is as defined in formula I above;
R_{N} is as defined in paragraph (48) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ib:
V₁ is as defined in formula I above;
R_{N} is as defined in paragraph (49) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ib:
V₁ is as defined in formula I above;
R_{N} is as defined in paragraph (50) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a further group of compounds of formula Ib defined herein:
V₁, R_{N}, R_{NA}, R₂ and L are as defined in any of the preceding paragraphs; and
Q₁ is as defined in paragraph (51) above.

In a further group of compounds of formula Ib defined herein:
V₁, R_{N}, R_{NA}, R₂ and L are as defined in any of the preceding paragraphs; and
Q₁ is as defined in paragraph (52) above.

In a further group of compounds of formula Ib defined herein:
V₁, R_{N}, R_{NA}, R₂ and L are as defined in any of the preceding paragraphs; and
Q₁ is as defined in paragraph (53) above.

In a further group of compounds of formula Ib defined herein:
V₁, R_{N}, R_{NA}, R₂ and L are as defined in any of the preceding paragraphs; and
Q₁ is as defined in paragraph (54) above.

In a further group of compounds of formula Ib defined herein:
V₁, R_{N}, R_{NA}, R₂ and L are as defined in any of the preceding paragraphs; and
Q₁ is as defined in paragraph (55) above.

In a particular group of compounds of the invention, the compound is a compound of formula I defined herein in which V₁ is C—R₂, i.e. the compounds have the formula Ic shown below, or a pharmaceutically acceptable salt thereof:

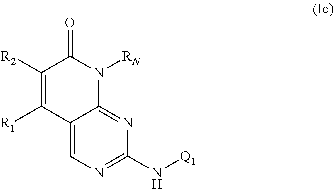

wherein R_{N}, R₁, R₂ and Q₁ each have any one of the definitions set out herein.

In a particular group of compounds of formula Ic:
R₁ is as defined in paragraph (11) above;
R_{N} is as defined in paragraph (1) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ic:
R₁ is as defined in paragraph (11) above;
R_{N} is as defined in paragraph (35) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ic:
R₁ is as defined in paragraph (11) above;
R_{N} is as defined in paragraph (38) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ic:
R₁ is as defined in paragraph (41) above;
R_{N} is as defined in paragraph (1) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ic:
R₁ is as defined in paragraph (44) above;
R_{N} is as defined in paragraph (1) above and R_{NA} is as defined in formula I above or is as defined any one of paragraphs (4), (5), (6), (7), (8), (9) or (10) above;
R₂ is as defined in paragraph (16) above;
Q₁ is as defined in paragraph (22) above; and
L is as defined in paragraph (28) above.

In a particular group of compounds of formula Ic:
R₁ is as defined in paragraph (11) above;
R_{N} is as defined in paragraph (2) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ic:
R₁ is as defined in paragraph (11) above;
R_{N} is as defined in paragraph (36) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ic:
R₁ is as defined in paragraph (11) above;
R_{N} is as defined in paragraph (39) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ic:
R₁ is as defined in paragraph (11) above;
R_{N} is as defined in paragraph (42) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ic:
R₁ is as defined in paragraph (11) above;
R_{N} is as defined in paragraph (45) above and R_{NA} is as defined in formula I above or is as defined any one of paragraphs (4), (5), (6), (7), (8), (9) or (10) above;
R₂ is as defined in paragraph (18) above;
Q₁ is as defined in paragraph (24) above; and
L is as defined in paragraph (30) above.

In a particular group of compounds of formula Ic:
R₁ is as defined in paragraph (11) above;
R_{N} is as defined in paragraph (3) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ic:
R₁ is as defined in paragraph (11) above;
R_{N} is as defined in paragraph (37) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ic:
R₁ is as defined in paragraph (11) above;
R_{N} is as defined in paragraph (40) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ic:
R₁ is as defined in paragraph (11) above;
R_{N} is as defined in paragraph (43) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ic:
R₁ is as defined in paragraph (11) above;
R_{N} is as defined in paragraph (46) above and R_{NA} is as defined in formula I above or is as defined any one of paragraphs (4), (5), (6), (7), (8), (9) or (10) above;
R₂ is as defined in paragraph (20) above;
Q₁ is as defined in paragraph (25) above; and
L is as defined in paragraph (32) above.

In a particular group of compounds of formula Ic:
R₁ is as defined in paragraph (11) above;
R_{N} is as defined in paragraph (3) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ic:
R₁ is as defined in paragraph (11) above;
R_{N} is as defined in paragraph (47) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ic:
R₁ is as defined in paragraph (11) above;
R_{N} is as defined in paragraph (48) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ic:
R₁ is as defined in paragraph (11) above;
R_{N} is as defined in paragraph (49) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a particular group of compounds of formula Ic:
R₁ is as defined in paragraph (11) above;
R_{N} is as defined in paragraph (50) above and R_{NA} is as defined in formula I above or is as defined any one of paragraphs (4), (5), (6), (7), (8), (9) or (10) above;
R₂ is as defined in paragraph (21) above;
Q₁ is as defined in paragraph (25) above; and
L is as defined in paragraph (34) above.

In a further group of compounds of formula Ic defined herein:
V₁, R_{N}, R_{NA}, R₁, R₂ and L are as defined in any of the preceding paragraphs; and
Q₁ is as defined in paragraph (51) above.

In a further group of compounds of formula Ic defined herein:
V₁, R_{N}, R_{NA}, R₁, R₂ and L are as defined in any of the preceding paragraphs; and
Q₁ is as defined in paragraph (52) above.

In a further group of compounds of formula Ic defined herein:
V₁, R_{N}, R_{NA}, R₁, R₂ and L are as defined in any of the preceding paragraphs; and
Q₁ is as defined in paragraph (53) above.

In a further group of compounds of formula Ic defined herein:
V₁, R_{N}, R_{NA}, R₁, R₂ and L are as defined in any of the preceding paragraphs; and
Q₁ is as defined in paragraph (54) above.

In a further group of compounds of formula Ic defined herein:
V₁, R_{N}, R_{NA}, R₁, R₂ and L are as defined in any of the preceding paragraphs; and
Q₁ is as defined in paragraph (55) above.

In a particular group of compounds of the invention, the compound is a compound of formula I defined herein in which V₁ is N, i.e. the compounds have the formula Id shown below, or a pharmaceutically acceptable salt thereof:

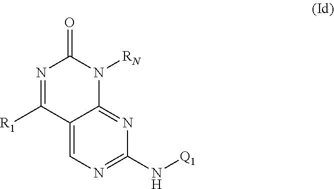

wherein R_{N}, R₁ and Q₁ each have any one of the definitions set out herein.

In a particular group of compounds of formula Id:
R₁ is as defined in paragraph (11) above;
R_{N} is as defined in paragraph (1) above and R_{NA} is as defined in formula I above or is as defined any one of paragraphs (4), (5), (6), (7), (8), (9) or (10) above;
Q₁ is as defined in paragraph (22) above; and
L is as defined in paragraph (28) above.

In a particular group of compounds of formula Id:
R₁ is as defined in paragraph (11) above;
R_{N} is as defined in paragraph (35) above and R_{NA} is as defined in formula I above or is as defined any one of paragraphs (4), (5), (6), (7), (8), (9) or (10) above;
Q₁ is as defined in paragraph (22) above; and
L is as defined in paragraph (28) above.

In a particular group of compounds of formula Id:
R₁ is as defined in paragraph (11) above;
R_{N} is as defined in paragraph (38) above and R_{NA} is as defined in formula I above or is as defined any one of paragraphs (4), (5), (6), (7), (8), (9) or (10) above;
Q₁ is as defined in paragraph (22) above; and
L is as defined in paragraph (28) above.

In a particular group of compounds of formula Id:
R₁ is as defined in paragraph (11) above;
R_{N} is as defined in paragraph (41) above and R_{NA} is as defined in formula I above or is as defined any one of paragraphs (4), (5), (6), (7), (8), (9) or (10) above;
Q₁ is as defined in paragraph (22) above; and
L is as defined in paragraph (28) above.

In a particular group of compounds of formula Id:
R₁ is as defined in paragraph (11) above;
R_{N} is as defined in paragraph (44) above and R_{NA} is as defined in formula I above or is as defined any one of paragraphs (4), (5), (6), (7), (8), (9) or (10) above;
Q₁ is as defined in paragraph (22) above; and
L is as defined in paragraph (28) above.

In a particular group of compounds of formula Id:
R₁ is as defined in paragraph (11) above;
R_{N} is as defined in paragraph (2) above and R_{NA} is as defined in formula I above or is as defined any one of paragraphs (4), (5), (6), (7), (8), (9) or (10) above;
Q₁ is as defined in paragraph (24) above; and
L is as defined in paragraph (30) above.

In a particular group of compounds of formula Id:
R₁ is as defined in paragraph (11) above;
R_{N} is as defined in paragraph (36) above and R_{NA} is as defined in formula I above or is as defined any one of paragraphs (4), (5), (6), (7), (8), (9) or (10) above;
Q₁ is as defined in paragraph (24) above; and
L is as defined in paragraph (30) above.

In a particular group of compounds of formula Id:
R₁ is as defined in paragraph (11) above;
R_{N} is as defined in paragraph (39) above and R_{NA} is as defined in formula I above or is as defined any one of paragraphs (4), (5), (6), (7), (8), (9) or (10) above;
Q₁ is as defined in paragraph (24) above; and
L is as defined in paragraph (30) above.

In a particular group of compounds of formula Id:
R₁ is as defined in paragraph (11) above;
R_{N} is as defined in paragraph (42) above and R_{NA} is as defined in formula I above or is as defined any one of paragraphs (4), (5), (6), (7), (8), (9) or (10) above;
Q₁ is as defined in paragraph (24) above; and
L is as defined in paragraph (30) above.

In a particular group of compounds of formula Id:
R₁ is as defined in paragraph (11) above;
R_{N} is as defined in paragraph (45) above and R_{NA} is as defined in formula I above or is as defined any one of paragraphs (4), (5), (6), (7), (8), (9) or (10) above;
Q₁ is as defined in paragraph (24) above; and
L is as defined in paragraph (30) above.

In a particular group of compounds of formula Id:
R₁ is as defined in paragraph (11) above;
R_{N} is as defined in paragraph (3) above and R_{NA} is as defined in formula I above or is as defined any one of paragraphs (4), (5), (6), (7), (8), (9) or (10) above;
Q₁ is as defined in paragraph (25) above; and
L is as defined in paragraph (32) above.

In a particular group of compounds of formula Id:
R₁ is as defined in paragraph (11) above;
R_{N} is as defined in paragraph (37) above and R_{NA} is as defined in formula I above or is as defined any one of paragraphs (4), (5), (6), (7), (8), (9) or (10) above;
Q₁ is as defined in paragraph (25) above; and
L is as defined in paragraph (32) above.

In a particular group of compounds of formula Id:
R₁ is as defined in paragraph (11) above;
R_{N} is as defined in paragraph (40) above and R_{NA} is as defined in formula I above or is as defined any one of paragraphs (4), (5), (6), (7), (8), (9) or (10) above;
Q₁ is as defined in paragraph (25) above; and
L is as defined in paragraph (32) above.

In a particular group of compounds of formula Id:
R₁ is as defined in paragraph (11) above;
R_{N} is as defined in paragraph (43) above and R_{NA} is as defined in formula I above or is as defined any one of paragraphs (4), (5), (6), (7), (8), (9) or (10) above;
Q₁ is as defined in paragraph (25) above; and
L is as defined in paragraph (32) above.

In a particular group of compounds of formula Id:
R₁ is as defined in paragraph (11) above;
R_{N} is as defined in paragraph (46) above and R_{NA} is as defined in formula I above or is as defined any one of paragraphs (4), (5), (6), (7), (8), (9) or (10) above;
Q₁ is as defined in paragraph (25) above; and
L is as defined in paragraph (32) above.

In a particular group of compounds of formula Id:
R₁ is as defined in paragraph (11) above;
R_{N} is as defined in paragraph (3) above and R_{NA} is as defined in formula I above or is as defined any one of paragraphs (4), (5), (6), (7), (8), (9) or (10) above;
Q₁ is as defined in paragraph (25) above; and
L is as defined in paragraph (34) above.

In a particular group of compounds of formula Id:
R₁ is as defined in paragraph (11) above;
R_{N} is as defined in paragraph (47) above and R_{NA} is as defined in formula I above or is as defined any one of paragraphs (4), (5), (6), (7), (8), (9) or (10) above;
Q₁ is as defined in paragraph (25) above; and
L is as defined in paragraph (34) above.

In a particular group of compounds of formula Id:
R₁ is as defined in paragraph (11) above;
R_{N} is as defined in paragraph (48) above and R_{NA} is as defined in formula I above or is as defined any one of paragraphs (4), (5), (6), (7), (8), (9) or (10) above;
Q₁ is as defined in paragraph (25) above; and
L is as defined in paragraph (34) above.

In a particular group of compounds of formula Id:
R₁ is as defined in paragraph (11) above;
R_{N} is as defined in paragraph (49) above and R_{NA} is as defined in formula I above or is as defined any one of paragraphs (4), (5), (6), (7), (8), (9) or (10) above;
Q₁ is as defined in paragraph (25) above; and
L is as defined in paragraph (34) above.

In a particular group of compounds of formula Id:
R₁ is as defined in paragraph (11) above;
R_{N} is as defined in paragraph (50) above and R_{NA} is as defined in formula I above or is as defined any one of paragraphs (4), (5), (6), (7), (8), (9) or (10) above;
Q₁ is as defined in paragraph (25) above; and
L is as defined in paragraph (34) above.

In a further group of compounds of formula Id defined herein:
R_{N}, R_{NA}, R₁, R₂ and L are as defined in any of the preceding paragraphs; and
Q₁ is as defined in paragraph (51) above.

In a further group of compounds of formula Id defined herein:
R_{N}, R_{NA}, R₁, R₂ and L are as defined in any of the preceding paragraphs; and
Q₁ is as defined in paragraph (52) above.

In a further group of compounds of formula Id defined herein:
R_{N}, R_{NA}, R₁, R₂ and L are as defined in any of the preceding paragraphs; and
Q₁ is as defined in paragraph (53) above.

In a further group of compounds of formula Id defined herein:
R_{N}, R_{NA}, R₁, R₂ and L are as defined in any of the preceding paragraphs; and
Q₁ is as defined in paragraph (54) above.

In a further group of compounds of formula Id defined herein:
R_{N}, R_{NA}, R₁, R₂ and L are as defined in any of the preceding paragraphs; and
Q₁ is as defined in paragraph (55) above.

In a particular group of compounds of the invention, the compound is a compound of formula I defined herein in which R_{N} and R₄ are linked to form a Linker group L, i.e. the compounds have the formula Ie shown below, or a pharmaceutically acceptable salt thereof:

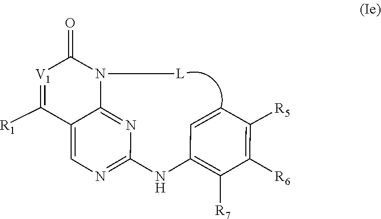

wherein R₁, V₁, R₅, R₆, R₇ and L each have any one of the definitions set out herein.

In a particular group of compounds of formula Ie:
R₁ is as defined in paragraph (11) above;
V₁ is as defined in formula I above;
R₂ is as defined in paragraph (16) above; and
R₅, R₆ and R₇ are as defined in paragraph (22)
L is as defined in paragraph (27) or (28) above.

In a particular group of compounds of formula Ie:
R₁ is as defined in paragraph (11) above;
V₁ is as defined in formula I above;
R₂ is as defined in paragraph (18) above; and
R₅, R₆ and R₇ are as defined in paragraph (23)
L is as defined in paragraph (29) above.

In a particular group of compounds of formula Ie:
R₁ is as defined in paragraph (11) above;
V₁ is as defined in formula I above;
R₂ is as defined in paragraph (19) above; and
R₅, R₆ and R₇ are as defined in paragraph (24)
L is as defined in paragraph (30) above.

In a particular group of compounds of formula Ie:
R₁ is as defined in paragraph (11) above;
V₁ is as defined in formula I above;
R₂ is as defined in paragraph (20) above; and
R₅, R₆ and R₇ are as defined in paragraph (24)
L is as defined in paragraph (31) above.

In a particular group of compounds of formula Ie:
R₁ is as defined in paragraph (11) above;
V₁ is as defined in formula I above;
R₂ is as defined in paragraph (20) above; and
R₅, R₆ and R₇ are as defined in paragraph (25)
L is as defined in paragraph (32) above.

In a particular group of compounds of formula Ie:
R₁ is as defined in paragraph (11) above;
V₁ is as defined in formula I above;
R₂ is as defined in paragraph (21) above; and
R₅, R₆ and R₇ are as defined in paragraph (26)
L is as defined in paragraph (33) above.

In a particular group of compounds of formula Ie:
R₁ is as defined in paragraph (11) above;
V₁ is as defined in formula I above;
R₂ is as defined in paragraph (21) above; and
R₅, R₆ and R₇ are as defined in paragraph (26)
L is as defined in paragraph (34) above.

In a further group of compounds of formula Ie defined herein:
V₁, R₁, R₂ and L are as defined in any of the preceding paragraphs; and
R₅, R₆ and R₇ are as defined in paragraph (51) above.

In a further group of compounds of formula Ie defined herein:
V₁, R₁, R₂ and L are as defined in any of the preceding paragraphs; and
R₅, R₆ and R₇ are as defined in paragraph (52) above.

In a further group of compounds of formula Ie defined herein:
V₁, R₁, R₂ and L are as defined in any of the preceding paragraphs; and
R₅, R₆ and R₇ are as defined in paragraph (53) above.

In a further group of compounds of formula Ie defined herein:
V₁, R₁, R₂ and L are as defined in any of the preceding paragraphs; and
R₅, R₆ and R₇ are as defined in paragraph (54) above.

In a further group of compounds of formula Ie defined herein:
V₁, R₁, R₂ and L are as defined in any of the preceding paragraphs; and
R₅, R₆ and R₇ are as defined in paragraph (55) above.

In a particular group of compounds of the invention, the compound is a compound of formula I defined herein having the structural formula If shown below, or a pharmaceutically acceptable salt thereof:

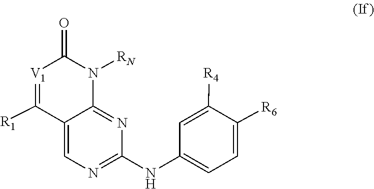

wherein R₁ is ethenyl or ethynyl, V₁, R_{N}, R₄ and R₅, each have any one of the definitions set out herein.

In a particular group of compounds of formula If, R₄ and R₅ are as defined in any one of paragraphs (22) to (26) and (51) to (55) above.

In a particular group of compounds of formula If,
V₁ is as defined in formula I above;
R₂ is as defined in paragraph (16) above;
R_{N} is as defined in paragraph (1) above and R_{NA} is as defined in formula I above or is as defined any one of paragraphs (4), (5), (6), (7), (8), (9) or (10) above;
R₄ and R₅ are as defined in any one of paragraphs (22) to (26) and (51) to (55) above.

In a particular group of compounds of formula If,
V₁ is as defined in formula I above;
R₂ is as defined in paragraph (16) above;
R_{N} is as defined in paragraph (2) above and R_{NA} is as defined in formula I above or is as defined any one of paragraphs (4), (5), (6), (7), (8), (9) or (10) above;
R₄ and R₅ are as defined in any one of paragraphs (22) to (26) and (51) to (55) above.

In a particular group of compounds of formula If,
V₁ is as defined in formula I above;
R₂ is as defined in paragraph (16) above;
R_{N} is as defined in paragraph (3) above and R_{NA} is as defined in formula I above or is as defined any one of paragraphs (4), (5), (6), (7), (8), (9) or (10) above;
R₄ and R₅ are as defined in any one of paragraphs (22) to (26) and (51) to (55) above.

In a particular group of compounds of formula If,
V₁ is as defined in formula I above;
R₂ is as defined in paragraph (16) above;
R_{N} is as defined in paragraph (35) above and R_{NA} is as defined in formula I above or is as defined any one of paragraphs (4), (5), (6), (7), (8), (9) or (10) above;
R₄ and R₅ are as defined in any one of paragraphs (22) to (26) and (51) to (55) above.

In a particular group of compounds of formula If,
V₁ is as defined in formula I above;
R₂ is as defined in paragraph (16) above;
R_{N} is as defined in paragraph (38) above and R_{NA} is as defined in formula I above or is as defined any one of paragraphs (4), (5), (6), (7), (8), (9) or (10) above;
R₄ and R₅ are as defined in any one of paragraphs (22) to (26) and (51) to (55) above.

In a particular group of compounds of formula If,
V₁ is as defined in formula I above;
R₂ is as defined in paragraph (16) above;
R_{N} is as defined in paragraph (44) above and R_{NA} is as defined in formula I above or is as defined any one of paragraphs (4), (5), (6), (7), (8), (9) or (10) above;
R₄ and R₅ are as defined in any one of paragraphs (22) to (26) and (51) to (55) above.

In a particular group of compounds of formula If,
V₁ is as defined in formula I above;
R₂ is as defined in paragraph (16) above;
R_{N} is as defined in paragraph (48) above and R_{NA} is as defined in formula I above or is as defined any one of paragraphs (4), (5), (6), (7), (8), (9) or (10) above;
R₄ and R₅ are as defined in any one of paragraphs (22) to (26) or (51) to (55) above.

Particular compounds of the present invention include any of the compounds described in the example section of the present application, or a pharmaceutically acceptable salt, hydrate or solvate thereof, and, in particular, any of the following:
5-ethynyl-2-((2-methoxyphenyl)amino)-8-phenylpyrido[2,3-d]pyrimidin-7(8H)-one
N-(2-((2-methoxyphenyl)amino)-7-oxo-8-phenyl-7,8-dihydropyrido[2,3-d]pyrimidin-5-yl)acrylamide
5-ethynyl-2-((2-methoxyphenyl)amino)-8-methylpyrido[2,3-d]pyrimidin-7(8H)-one
8-cyclopentyl-5-ethynyl-2-((2-methoxyphenyl)amino)pyrido[2,3-d]pyrimidin-7(8H)-one
8-(2,4-dimethoxyphenyl)-5-ethynyl-2-((2-methoxyphenyl)amino)pyrido[2,3-d]pyrimidin-7(8H)-one
N-(4-((5-ethynyl-7-oxo-8-phenyl-7,8-dihydropyrido[2,3-d]pyrimidin-2-yl)amino)-3-methoxyphenyl)-2-methoxy-N-methylacetamide
5-ethynyl-2-[(2-methoxyphenyl)amino]-N-methyl-7-oxo-8-phenylpyrido[2,3-d]pyrimidine-6-carboxamide
5-ethynyl-2-((2-methoxyphenyl)amino)-7-oxo-8-phenyl-7,8-dihydropyrido[2,3-d]pyrimidine-6-carbonitrile
5-ethynyl-2-((2-methoxyphenyl)amino)-N,N,8-trimethyl-7-oxo-7,8-dihydropyrido[2,3-d]pyrimidine-6-carboxamide
5-ethynyl-8-isopropyl-2-((2-methoxyphenyl)amino)pyrido[2,3-d]pyrimidin-7(8H)-one
5-ethynyl-8-(4-methoxyphenyl)-2-((2-methoxyphenyl)amino)pyrido[2,3-d]pyrimidin-7(8H)-one
2-((2-methoxyphenyl)amino)-8-phenyl-5-vinylpyrido[2,3-d]pyrimidin-7(8H)-one
N-(3-(5-ethynyl-2-((2-methoxyphenyl)amino)-7-oxopyrido[2,3-d]pyrimidin-8(7H)-yl)phenyl)acetamide
N-(3-(5-ethynyl-2-((2-methoxyphenyl)amino)-7-oxopyrido[2,3-d]pyrimidin-8(7H)-yl)phenyl)methanesulfonamide
2-((2-methoxyphenyl)amino)-7-oxo-8-phenyl-5-vinyl-7,8-dihydropyrido[2,3-d]pyrimidine-6-carbonitrile
8-(2,4-dimethoxyphenyl)-2-((2-methoxyphenyl)amino)-5-vinylpyrido[2,3-d]pyrimidin-7(8H)-one
6-(dimethylamino)-5-ethynyl-2-((2-methoxyphenyl)amino)-8-phenylpyrido[2,3-d]pyrimidin-7(8H)-one
8-(2,4-dimethoxyphenyl)-2-((2-methoxyphenyl)amino)-7-oxo-5-vinyl-7,8-dihydropyrido[2,3-d]pyrimidine-6-carbonitrile
6-amino-5-ethynyl-2-((2-methoxyphenyl)amino)-8-phenylpyrido[2,3-d]pyrimidin-7(8H)-one
N-(5-ethynyl-2-((2-methoxyphenyl)amino)-7-oxo-8-phenyl-7,8-dihydropyrido[2,3-d]pyrimidin-6-yl)acetamide
N-(3-(5-ethynyl-2-((2-methoxyphenyl)amino)-7-oxopyrido[2,3-d]pyrimidin-8(7H)-yl)phenyl)acrylamide
5-ethynyl-8-phenyl-2-(phenylamino)pyrido[2,3-d]pyrimidin-7(8H)-one
6-(2-chlorophenyl)-2-((4-((2-(dimethylamino)ethyl)(methyl)amino)-2-methoxyphenyl)amino)-5-ethynyl-8-methylpyrido[2,3-d]pyrimidin-7(8H)-one
2-((4-((2-(dimethylamino)ethyl)(methyl)amino)-2-methoxyphenyl)amino)-5-ethynyl-8-methyl-6-phenylpyrido[2,3-d]pyrimidin-7(8H)-one
2-((4-((2-(dimethylamino)ethyl)(methyl)amino)-2-methoxyphenyl)amino)-5-ethynyl-8-methylpyrido[2,3-d]pyrimidin-7(8H)-one
6-benzyl-2-((4-((2-(dimethylamino)ethyl)(methyl)amino)-2-methoxyphenyl)amino)-5-ethynyl-8-methylpyrido[2,3-d]pyrimidin-7(8H)-one
4-ethynyl-7-((2-methoxyphenyl)amino)-1-phenylpyrimido[4,5-d]pyrimidin-2(1H)-one
2-((4-((2-(dimethylamino)ethyl)(methyl)amino)phenyl)amino)-5-ethynyl-8-phenylpyrido[2,3-d]pyrimidin-7(8H)-one
8-(2-methoxyphenyl)-2-((2-methoxyphenyl)amino)-5-vinylpyrido[2,3-d]pyrimidin-7(8H)-one
5-ethynyl-2-((2-methoxy-4-morpholinophenyl)amino)-8-phenylpyrido[2,3-d]pyrimidin-7(8H)-one
6-(2-chlorophenyl)-5-ethynyl-2-((2-methoxyphenyl)amino)-8-methylpyrido[2,3-d]pyrimidin-7(8H)-one
6-(2-chlorophenyl)-2-((2-methoxyphenyl)amino)-8-methyl-5-vinylpyrido[2,3-d]pyrimidin-7(8H)-one
2-((2-methoxyphenyl)amino)-8-methyl-6-phenyl-5-vinylpyrido[2,3-d]pyrimidin-7(8H)-one
6-(2-chlorophenyl)-5-ethynyl-8-methyl-2-(phenylamino)pyrido[2,3-d]pyrimidin-7(8H)-one
6-(2,6-dichlorophenyl)-2-((2-methoxyphenyl)amino)-8-methyl-5-vinylpyrido[2,3-d]pyrimidin-7(8H)-one
6-(2,6-dichlorophenyl)-5-ethynyl-2-((3-(hydroxymethyl)phenyl)amino)-8-methylpyrido[2,3-d]pyrimidin-7(8H)-one
6-(2,6-dichlorophenyl)-2-((3-(hydroxymethyl)phenyl)amino)-8-methyl-5-vinylpyrido[2,3-d]pyrimidin-7(8H)-one
6-(2-chlorophenyl)-2-((4-((2-(dimethylamino)ethyl)(methyl)amino)phenyl)amino)-5-ethynyl-8-methylpyrido[2,3-d]pyrimidin-7(8H)-one
6-(2-chlorophenyl)-2-((4-((2-(dimethylamino)ethyl)(methyl)amino)phenyl)amino)-8-methyl-5-vinylpyrido[2,3-d]pyrimidin-7(8H)-one
6-benzyl-2-((4-((2-(dimethylamino)ethyl)(methyl)amino)phenyl)amino)-8-methyl-5-vinylpyrido[2,3-d]pyrimidin-7(8H)-one
6-(2,6-dichlorophenyl)-2-((4-((2-(dimethylamino)ethyl)(methyl)amino)phenyl)amino)-8-methyl-5-vinylpyrido[2,3-d]pyrimidin-7(8H)-one
6-(2-chlorophenyl)-2-((3-methoxyphenyl)amino)-8-methyl-5-vinylpyrido[2,3-d]pyrimidin-7(8H)-one
6-(2-chlorophenyl)-2-((3-(hydroxymethyl)phenyl)amino)-8-methyl-5-vinylpyrido[2,3-d]pyrimidin-7(8H)-one
N-(3-(5-ethynyl-2-((2-methoxyphenyl)amino)-7-oxopyrido[2,3-d]pyrimidin-8(7H)-yl)phenyl)cyclopropanesulfonamide
N-(4-((5-ethynyl-7-oxo-8-phenyl-7,8-dihydropyrido[2,3-d]pyrimidin-2-yl)amino)-3-methoxyphenyl)-3-methoxy-N-methylpropanamide
6-(2-chlorophenyl)-5-ethynyl-2-((2-methoxy-4-(4-methylpiperazin-1-yl)phenyl)amino)-8-methylpyrido[2,3-d]pyrimidin-7(8H)-one
6-(2-chlorophenyl)-5-ethynyl-2-((3-(hydroxymethyl)phenyl)amino)-8-methylpyrido[2,3-d]pyrimidin-7(8H)-one
6-(2-chlorophenyl)-2-((2-methoxy-4-(4-methylpiperazin-1-yl)phenyl)amino)-8-methyl-5-vinylpyrido[2,3-d]pyrimidin-7(8H)-one
N-(2-((2-methoxyphenyl)amino)-7-oxo-8-phenyl-5-vinyl-7,8-dihydropyrido[2,3-d]pyrimidin-6-yl)acetamide
6-(2,6-dichlorophenyl)-2-((4-((2-(dimethylamino)ethyl)(methyl)amino)phenyl)amino)-5-ethynyl-8-methylpyrido[2,3-d]pyrimidin-7(8H)-one
6-(2-chlorophenyl)-5-ethynyl-8-methyl-2-((4-(4-methylpiperazin-1-yl)phenyl)amino)pyrido[2,3-d]pyrimidin-7(8H)-one
6-(2-chlorophenyl)-2-((4-(2-(dimethylamino)ethoxy)phenyl)amino)-5-ethynyl-8-methylpyrido[2,3-d]pyrimidin-7(8H)-one
6-(2-chlorophenyl)-5-ethynyl-2-((3-methoxyphenyl)amino)-8-methylpyrido[2,3-d]pyrimidin-7(8H)-one
6-(2-chlorophenyl)-8-methyl-2-((4-(4-methylpiperazin-1-yl)phenyl)amino)-5-vinylpyrido[2,3-d]pyrimidin-7(8H)-one
6-(2-chlorophenyl)-2-((4-(2-(dimethylamino)ethoxy)phenyl)amino)-8-methyl-5-vinylpyrido[2,3-d]pyrimidin-7(8H)-one
2-amino-6-(2-chlorophenyl)-8-methyl-5-vinylpyrido[2,3-d]pyrimidin-7(8H)-one
4-ethynyl-7-((2-methoxyphenyl)amino)-1-phenyl-1,6-naphthyridin-2(1H)-one
5-ethynyl-2-((4-(4-methylpiperazin-1-yl)phenyl)amino)-8-phenylpyrido[2,3-d]pyrimidin-7(8H)-one
(R)-2-((4-((2-(dimethylamino)ethyl)(methyl)amino)phenyl)amino)-5-ethynyl-8-(1-propionylpiperidin-3-yl)pyrido[2,3-d]pyrimidin-7(8H)-one
6-(2-chlorophenyl)-5-ethynyl-8-methyl-2-((4-(4-methylpiperazin-1-yl)butyl)amino)pyrido[2,3-d]pyrimidin-7(8H)-one
6-(2-chlorophenyl)-8-methyl-2-((4-(4-methylpiperazin-1-yl)butyl)amino)-5-vinylpyrido[2,3-d]pyrimidin-7(8H)-one
5-ethynyl-8-methyl-2-((4-(4-methylpiperazin-1-yl)phenyl)amino)pyrido[2,3-d]pyrimidin-7(8H)-one
2-((4-(2-(dimethylamino)ethoxy)phenyl)amino)-5-ethynyl-8-methylpyrido[2,3-d]pyrimidin-7(8H)-one
2-((4-((2-(dimethylamino)ethyl)(methyl)amino)phenyl)amino)-5-ethynyl-8-methylpyrido[2,3-d]pyrimidin-7(8H)-one
5-ethynyl-2-((2-methoxy-4-(4-methylpiperazin-1-yl)phenyl)amino)-8-methylpyrido[2,3-d]pyrimidin-7(8H)-one
N-((1s,4s)-4-(2-((4-((2-(dimethylamino)ethyl)(methyl)amino)phenyl)amino)-5-ethynyl-7-oxopyrido[2,3-d]pyrimidin-8(7H)-yl)cyclohexyl)acetamide
2-((4-((2-(dimethylamino)ethyl)(methyl)amino)phenyl)amino)-5-ethynyl-8-((1s,4s)-4-(hydroxymethyl)cyclohexyl)pyrido[2,3-d]pyrimidin-7(8H)-one
6-(2-chlorophenyl)-2-((4-((2-(dimethylamino)ethyl)(methyl)amino)-3-methylphenyl)amino)-5-ethynyl-8-methylpyrido[2,3-d]pyrimidin-7(8H)-one
8-(2,4-dimethoxyphenyl)-2-((4-((2-(dimethylamino)ethyl)(methyl)amino)phenyl)amino)-5-ethynylpyrido[2,3-d]pyrimidin-7(8H)-one
2-((4-((2-(dimethylamino)ethyl)(methyl)amino)-3-(1-methyl-1H-pyrazol-4-yl)phenyl)amino)-5-ethynyl-8-methylpyrido[2,3-d]pyrimidin-7(8H)-one
6-(2-chloro-5-methoxyphenyl)-2-((4-((2-(dimethylamino)ethyl)(methyl)amino)phenyl)amino)-5-ethynyl-8-methylpyrido[2,3-d]pyrimidin-7(8H)-one
6-(2-chloro-5-fluorophenyl)-2-((4-((2-(dimethylamino)ethyl)(methyl)amino)phenyl)amino)-5-ethynyl-8-methylpyrido[2,3-d]pyrimidin-7(8H)-one
5-ethynyl-8-methyl-2-((4-(4-methylpiperazin-1-yl)phenyl)amino)pyrido[2,3-d]pyrimidin-7(8H)-one
2-((4-(2-(dimethylamino)ethoxy)phenyl)amino)-5-ethynyl-8-phenylpyrido[2,3-d]pyrimidin-7(8H)-one
5-ethynyl-2-((2-methoxyphenyl)amino)pyrido[2,3-d]pyrimidin-7(8H)-one
8-(2,4-dimethoxyphenyl)-2-((4-((2-(dimethylamino)ethyl)(methyl)amino)phenyl)amino)-5-vinylpyrido[2,3-d]pyrimidin-7(8H)-one
6-(2-chloro-3-fluorophenyl)-2-((4-((2-(dimethylamino)ethyl)(methyl)amino)phenyl)amino)-5-ethynyl-8-methylpyrido[2,3-d]pyrimidin-7(8H)-one
2-((4-(4-(dimethylamino)piperidin-1-yl)phenyl)amino)-5-ethynyl-8-phenylpyrido[2,3-d]pyrimidin-7(8H)-one
2-((4-((2-(dimethylamino)ethyl)(methyl)amino)phenyl)amino)-5-ethynyl-6-(2-methoxyphenyl)-8-methylpyrido[2,3-d]pyrimidin-7(8H)-one
2-((4-((2-(dimethylamino)ethyl)(methyl)amino)phenyl)amino)-5-ethynyl-8-methyl-6-(phenylamino)pyrido[2,3-d]pyrimidin-7(8H)-one
5-ethynyl-8-phenyl-2-((4-(piperazin-1-yl)phenyl)amino)pyrido[2,3-d]pyrimidin-7(8H)-one
2-((4-(4-ethylpiperazin-1-yl)phenyl)amino)-5-ethynyl-8-phenylpyrido[2,3-d]pyrimidin-7(8H)-one
5-ethynyl-2-((4-(4-isopropylpiperazin-1-yl)phenyl)amino)-8-phenylpyrido[2,3-d]pyrimidin-7(8H)-one
5-ethynyl-2-((4-(4-(2-hydroxyethyl)piperazin-1-yl)phenyl)amino)-8-phenylpyrido[2,3-d]pyrimidin-7(8H)-one
5-ethynyl-2-((4-(4-(oxetan-3-yl)piperazin-1-yl)phenyl)amino)-8-phenylpyrido[2,3-d]pyrimidin-7(8H)-one
5-ethynyl-2-((4-(1-methylpiperidin-4-yl)phenyl)amino)-8-phenylpyrido[2,3-d]pyrimidin-7(8H)-one
2-((4-((2-(dimethylamino)ethyl)(methyl)amino)-3-methylphenyl)amino)-5-ethynyl-8-phenylpyrido[2,3-d]pyrimidin-7(8H)-one
6-(3-chloropyridin-4-yl)-2-((4-((2-(dimethylamino)ethyl)(methyl)amino)phenyl)amino)-5-ethynyl-8-methylpyrido[2,3-d]pyrimidin-7(8H)-one
2-((4-(4,7-diazaspiro[2.5]octan-7-yl)phenyl)amino)-5-ethynyl-8-phenylpyrido[2,3-d]pyrimidin-7(8H)-one
5-ethynyl-2-((1-(1-methylpiperidin-4-yl)-1H-pyrazol-4-yl)amino)-8-phenylpyrido[2,3-d]pyrimidin-7(8H)-one
2-((4-(1,4-diazepan-1-yl)phenyl)amino)-5-ethynyl-8-phenylpyrido[2,3-d]pyrimidin-7(8H)-one
5-ethynyl-8-phenyl-2-((4-(piperidin-4-yl)phenyl)amino)pyrido[2,3-d]pyrimidin-7(8H)-one
5-ethynyl-2-((4-(4-(4-methylpiperazin-1-yl)piperidin-1-yl)phenyl)amino)-8-phenylpyrido[2,3-d]pyrimidin-7(8H)-one
5-ethynyl-8-phenyl-2-((2,3,4,5-tetrahydro-1H-benzo[d]azepin-7-yl)amino)pyrido[2,3-d]pyrimidin-7(8H)-one
5-ethynyl-2-((4-(3-methylpiperazin-1-yl)phenyl)amino)-8-phenylpyrido[2,3-d]pyrimidin-7(8H)-one
8-(3-methoxyphenyl)-2-((2-methoxyphenyl)amino)-5-vinylpyrido[2,3-d]pyrimidin-7(8H)-one
5-ethynyl-2-((2-methoxyphenyl)amino)-8-(3-nitrophenyl)pyrido[2,3-d]pyrimidin-7(8H)-one
5-ethynyl-2-((2-methoxyphenyl)amino)-8-(4-nitrophenyl)pyrido[2,3-d]pyrimidin-7(8H)-one
8-(3-aminophenyl)-5-ethynyl-2-((2-methoxyphenyl)amino)pyrido[2,3-d]pyrimidin-7(8H)-one
5-ethynyl-2-((1-methyl-1H-pyrazol-4-yl)amino)-8-phenylpyrido[2,3-d]pyrimidin-7(8H)-one
5-ethynyl-2-((3-(hydroxymethyl)-4-(4-methylpiperazin-1-yl)phenyl)amino)-8-phenylpyrido[2,3-d]pyrimidin-7(8H)-one
5-ethynyl-2-((4-(4-(3-(hydroxymethyl)azetidin-1-yl)piperidin-1-yl)phenyl)amino)-8-phenylpyrido[2,3-d]pyrimidin-7(8H)-one
5-ethynyl-2-((4-(4-(3-fluoro-3-(hydroxymethyl)azetidin-1-yl)piperidin-1-yl)phenyl)amino)-8-phenylpyrido[2,3-d]pyrimidin-7(8H)-one
2-((4-(4-(3-(dimethylamino)azetidin-1-yl)piperidin-1-yl)phenyl)amino)-5-ethynyl-8-phenylpyrido[2,3-d]pyrimidin-7(8H)-one
5-ethynyl-2-((2-methoxy-5-(1-methyl-1H-pyrazol-4-yl)-4-morpholinophenyl)amino)-8-methylpyrido[2,3-d]pyrimidin-7(8H)-one
N-(5-((5-ethynyl-8-methyl-7-oxo-7,8-dihydropyrido[2,3-d]pyrimidin-2-yl)amino)-2-(4-methylpiperazin-1-yl)phenyl)acetamide
N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)propionamide
N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)isobutyramide
2-((3-(5-ethynyl-2-((2-methoxyphenyl)amino)-7-oxopyrido[2,3-d]pyrimidin-8(7H)-yl)phenyl)amino)-2-oxoethyl acetate
8-(4-aminophenyl)-5-ethynyl-2-((2-methoxyphenyl)amino)pyrido[2,3-d]pyrimidin-7(8H)-one
N-(4-(5-ethynyl-2-((2-methoxyphenyl)amino)-7-oxopyrido[2,3-d]pyrimidin-8(7H)-yl)phenyl)acetamide
2-((4-((3-(Dimethylamino)propyl)(methyl)amino)phenyl)amino)-5-ethynyl-8-phenylpyrido[2,3-d]pyrimidin-7(8H)-one
2-((3-ethyl-4-(4-methylpiperazin-1-yl)phenyl)amino)-5-ethynyl-8-phenylpyrido[2,3-d]pyrimidin-7(8H)-one
5-ethynyl-8-isopropyl-2-((4-(4-methylpiperazin-1-yl)phenyl)amino)pyrido[2,3-d]pyrimidin-7(8H)-one
8-cyclopentyl-5-ethynyl-2-((4-(4-methylpiperazin-1-yl)phenyl)amino)pyrido[2,3-d]pyrimidin-7(8H)-one
N-(5-((5-ethynyl-8-methyl-7-oxo-7,8-dihydropyrido[2,3-d]pyrimidin-2-yl)amino)-2-(4-methylpiperazin-1-yl)phenyl)propionamide
5-ethynyl-2-((1-(methylsulfonyl)piperidin-4-yl)amino)-8-phenylpyrido[2,3-d]pyrimidin-7(8H)-one
5-ethynyl-2-((4-morpholinophenyl)amino)-8-phenylpyrido[2,3-d]pyrimidin-7(8H)-one
N-(4-(5-ethynyl-2-((2-methoxyphenyl)amino)-7-oxopyrido[2,3-d]pyrimidin-8(7H)-yl)phenyl)methanesulfonamide
N-(4-(5-ethynyl-2-((2-methoxyphenyl)amino)-7-oxopyrido[2,3-d]pyrimidin-8(7H)-yl)phenyl)cyclopropanecarboxamide
5-ethynyl-2-((4-(4-(3-(methoxymethyl)azetidin-1-yl)piperidin-1-yl)phenyl)amino)-8-phenylpyrido[2,3-d]pyrimidin-7(8H)-one
N-((1r,4r)-4-(5-ethynyl-2-((4-(4-methylpiperazin-1-yl)phenyl)amino)-7-oxopyrido[2,3-d]pyrimidin-8(7H)-yl)cyclohexyl)acetamide
2-((4-(5-ethynyl-2-((2-methoxyphenyl)amino)-7-oxopyrido[2,3-d]pyrimidin-8(7H)-yl)phenyl)amino)-2-oxoethyl acetate
N-(4-(5-ethynyl-2-((2-methoxyphenyl)amino)-7-oxopyrido[2,3-d]pyrimidin-8(7H)-yl)phenyl)acrylamide
8-(4-methoxyphenyl)-2-((2-methoxyphenyl)amino)-5-vinylpyrido[2,3-d]pyrimidin-7(8H)-one
6-cyclopropyl-5-ethynyl-8-methyl-2-((4-(4-methylpiperazin-1-yl)phenyl)amino)pyrido[2,3-d]pyrimidin-7(8H)-one
5-ethynyl-6,8-dimethyl-2-((4-(4-methylpiperazin-1-yl)phenyl)amino)pyrido[2,3-d]pyrimidin-7(8H)-one
5-ethynyl-2-((4-(4-(3-(2-hydroxypropan-2-yl)azetidin-1-yl)piperidin-1-yl)phenyl)amino)-8-phenylpyrido[2,3-d]pyrimidin-7(8H)-one
2-((3-((2-(dimethylamino)ethyl)(methyl)amino)phenyl)amino)-5-ethynyl-8-methylpyrido[2,3-d]pyrimidin-7(8H)-one
N-(4-(5-ethynyl-2-((2-methoxyphenyl)amino)-7-oxopyrido[2,3-d]pyrimidin-8(7H)-yl)phenyl)propionamide
5-ethynyl-2-((2-(hydroxymethyl)phenyl)amino)-8-phenylpyrido[2,3-d]pyrimidin-7(8H)-one
5-ethynyl-8-methyl-2-((4-(4-methylpiperazin-1-yl)phenyl)amino)-6-(thiophen-2-yl)pyrido[2,3-d]pyrimidin-7(8H)-one
2-(dimethylamino)-N-(3-((5-ethynyl-8-methyl-7-oxo-7,8-dihydropyrido[2,3-d]pyrimidin-2-yl)amino)phenyl)-N-methylacetamide
N-((1r,4r)-4-(5-ethynyl-2-((4-(4-methylpiperazin-1-yl)phenyl)amino)-7-oxopyrido[2,3-d]pyrimidin-8(7H)-yl)cyclohexyl)pyridazine-4-carboxamide
2-(dimethylamino)-N-(4-((5-ethynyl-7-oxo-8-phenyl-7,8-dihydropyrido[2,3-d]pyrimidin-2-yl)amino)-3-methoxyphenyl)acetamide
4-(((1s,4s)-4-(5-ethynyl-2-((4-(4-methylpiperazin-1-yl)phenyl)amino)-7-oxopyrido[2,3-d]pyrimidin-8(7H)-yl)cyclohexyl)amino)-4-oxobutanoic acid
N1-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-N4,N4-dimethylsuccinamide
N1-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-N3-methylmalonamide
N1-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-N3,N3-dimethylmalonamide
3-(dimethylamino)-N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)propanamide
4-(dimethylamino)-N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)butanamide
N1-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-N4-methylsuccinamide
3-(((1s,4s)-4-(5-ethynyl-2-((4-(4-methylpiperazin-1-yl)phenyl)amino)-7-oxopyrido[2,3-d]pyrimidin-8(7H)-yl)cyclohexyl)amino)-3-oxopropanoic acid
2-(dimethylamino)-N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)acetamide
5-ethynyl-6-methyl-2-((4-(4-methylpiperazin-1-yl)phenyl)amino)-8-phenylpyrido[2,3-d]pyrimidin-7(8H)-one
8-(cyclopentylmethyl)-5-ethynyl-2-((4-(4-methylpiperazin-1-yl)phenyl)amino)pyrido[2,3-d]pyrimidin-7(8H)-one
5-ethynyl-2-((1′-methyl-[1,4′-b]piperidin-4-yl)amino)-8-phenylpyrido[2,3-d]pyrimidin-7(8H)-one
8-cyclohexyl-5-ethynyl-2-((4-(4-methylpiperazin-1-yl)phenyl)amino)pyrido[2,3-d]pyrimidin-7(8H)-one
5-ethynyl-2-((4-(4-methylpiperazin-1-yl)phenyl)amino)-8-(tetrahydro-2H-pyran-4-yl)pyrido[2,3-d]pyrimidin-7(8H)-one
5-ethynyl-2-((4-(4-methylpiperazin-1-yl)phenyl)amino)-8-((tetrahydrofuran-3-yl)methyl)pyrido[2,3-d]pyrimidin-7(8H)-one
(S)-5-ethynyl-2-((4-(4-methylpiperazin-1-yl)phenyl)amino)-8-((5-oxopyrrolidin-2-yl)methyl)pyrido[2,3-d]pyrimidin-7(8H)-one
5-ethynyl-2-((4-(4-methylpiperazin-1-yl)phenyl)amino)-8-((tetrahydrofuran-2-yl)methyl)pyrido[2,3-d]pyrimidin-7(8H)-one
N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-2-(1H-pyrazol-4-yl)acetamide
2-(dimethylamino)-N-((1s,4s)-4-(5-ethynyl-2-((2-methoxyphenyl)amino)-7-oxopyrido[2,3-d]pyrimidin-8(7H)-yl)cyclohexyl)acetamide
5-ethynyl-8-(2-hydroxycyclopentyl)-2-((4-(4-methylpiperazin-1-yl)phenyl)amino)pyrido[2,3-d]pyrimidin-7(8H)-one
2-(dimethylamino)-N-((1s,4s)-4-(5-ethynyl-2-((2-methoxyphenyl)amino)-7-oxopyrido[2,3-d]pyrimidin-8(7H)-yl)cyclohexyl)-N-methylacetamide
N-((1r,4r)-4-(5-ethynyl-6-methyl-2-((4-(4-methylpiperazin-1-yl)phenyl)amino)-7-oxopyrido[2,3-d]pyrimidin-8(7H)-yl)cyclohexyl)acetamide
N-((1s,4s)-4-(5-ethynyl-6-methyl-2-((4-(4-methylpiperazin-1-yl)phenyl)amino)-7-oxopyrido[2,3-d]pyrimidin-8(7H)-yl)cyclohexyl)acetamide
5-ethynyl-2-((4-(4-methylpiperazin-1-yl)phenyl)amino)-8-(2-oxopyrrolidin-3-yl)pyrido[2,3-d]pyrimidin-7(8H)-one
5-ethynyl-2-((4-(4-methylpiperazin-1-yl)phenyl)amino)-8-(tetrahydrofuran-3-yl)pyrido[2,3-d]pyrimidin-7(8H)-one
5-ethynyl-2-((4-(4-methylpiperazin-1-yl)phenyl)amino)-8-(pyrrolidin-3-yl)pyrido[2,3-d]pyrimidin-7(8H)-one
5-ethynyl-2-((4-(4-methylpiperazin-1-yl)phenyl)amino)-8-(1-methylpyrrolidin-3-yl)pyrido[2,3-d]pyrimidin-7(8H)-one
5-ethynyl-2-((4-(4-methylpiperazin-1-yl)phenyl)amino)-8-(5-oxopyrrolidin-3-yl)pyrido[2,3-d]pyrimidin-7(8H)-one
8-((1H-pyrazol-5-yl)methyl)-5-ethynyl-6-methyl-2-((4-(4-methylpiperazin-1-yl)phenyl)amino)pyrido[2,3-d]pyrimidin-7(8H)-one
5-ethynyl-6-methyl-8-((1-methyl-1H-pyrazol-3-yl)methyl)-2-((4-(4-methylpiperazin-1-yl)phenyl)amino)pyrido[2,3-d]pyrimidin-7(8H)-one
8-((1H-imidazol-5-yl)methyl)-5-ethynyl-6-methyl-2-((4-(4-methylpiperazin-1-yl)phenyl)amino)pyrido[2,3-d]pyrimidin-7(8H)-one
5-ethynyl-6-methyl-8-((1-methyl-1H-pyrazol-5-yl)methyl)-2-((4-(4-methylpiperazin-1-yl)phenyl)amino)pyrido[2,3-d]pyrimidin-7(8H)-one
5-ethynyl-8-(isoxazol-5-ylmethyl)-6-methyl-2-((4-(4-methylpiperazin-1-yl)phenyl)amino)pyrido[2,3-d]pyrimidin-7(8H)-one
5-ethynyl-6-methyl-2-((4-(4-methylpiperazin-1-yl)phenyl)amino)-8-(pyridin-3-ylmethyl)pyrido[2,3-d]pyrimidin-7(8H)-one
8-(cyclopentylmethyl)-5-ethynyl-6-methyl-2-((4-(4-methylpiperazin-1-yl)phenyl)amino)pyrido[2,3-d]pyrimidin-7(8H)-one
5-ethynyl-8-(1-methyl-5-oxopyrrolidin-3-yl)-2-((4-(4-methylpiperazin-1-yl)phenyl)amino)pyrido[2,3-d]pyrimidin-7(8H)-one
5-ethynyl-8-((1-methyl-1H-pyrazol-3-yl)methyl)-2-((4-(4-methylpiperazin-1-yl)phenyl)amino)pyrido[2,3-d]pyrimidin-7(8H)-one
5-ethynyl-6-methyl-2-((4-(4-methylpiperazin-1-yl)phenyl)amino)-8-(oxazol-2-ylmethyl)pyrido[2,3-d]pyrimidin-7(8H)-one
5-ethynyl-2-((4-(4-methylpiperazin-1-yl)phenyl)amino)-8-((2-oxopyrrolidin-3-yl)methyl)pyrido[2,3-d]pyrimidin-7(8H)-one
1⁵-ethynyl-1⁷,1⁸-dihydro-4-oxa-2-aza-1(2,8)-pyrido[2,3-d]pyrimidina-3(1,3)-benzenacycloheptaphan-1⁷-one
(Z)-1⁵-ethynyl-1⁷,1⁸-dihydro-4-oxa-2-aza-1(2,8)-pyrido[2,3-d]pyrimidina-3(1,3)-benzenacyclooctaphan-6-en-1⁷-one
(Z)-1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-6-en-1⁷-one
1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-1⁷-one;
5-Ethynyl-6-methyl-2-{[4-(4-methylpiperazin-1-yl)phenyl]amino}-8-(1,2-oxazol-4-ylmethyl)pyrido[2,3-d]pyrimidin-7-one;
5-Ethynyl-6-methyl-2-{[4-(4-methylpiperazin-1-yl)phenyl]amino}-8-(1,3-oxazol-5-ylmethyl)pyrido[2,3-d]pyrimidin-7-one;
5-Ethynyl-8-(1H-imidazol-2-ylmethyl)-6-methyl-2-{[4-(4-methylpiperazin-1-yl)phenyl]amino}pyrido[2,3-d]pyrimidin-7-one;
5-Ethynyl-6-methyl-2-{[4-(4-methylpiperazin-1-yl)phenyl]amino}-8-(pyridin-2-ylmethyl)pyrido[2,3-d]pyrimidin-7-one;
5-Ethynyl-6-methyl-2-{[4-(4-methylpiperazin-1-yl)phenyl]amino}-8-(pyridin-4-ylmethyl)pyrido[2,3-d]pyrimidin-7-one;
5-Ethynyl-6-methyl-2-{[4-(4-methylpiperazin-1-yl)phenyl]amino}-8-[(1-methylpyrazol-4-yl)methyl]pyrido[2,3-d]pyrimidin-7-one;
8-Cyclopentyl-5-ethynyl-2-[(3-([3-(methanesulfonylmethyl)azetidin-1-yl]methylphenyl)amino]pyrido[2,3-d]pyrimidin-7-one;
8-Cyclopentyl-5-ethynyl-2-[(3-([3-(methanesulfonylmethyl)pyrrolidin-1-yl]methylphenyl)amino]pyrido[2,3-d]pyrimidin-7-one;
8-Cyclopentyl-5-ethynyl-2-[(3-([4-(methanesulfonylmethyl)piperidin-1-yl]methylphenyl)amino]pyrido[2,3-d]pyrimidin-7-one;
8-Cyclopentyl-5-ethynyl-2-[(2-methyl-1,3-dihydroisoindol-4-yl)amino]pyrido[2,3-d]pyrimidin-7-one;
8-Cyclopentyl-5-ethynyl-2-[(2-methyl-3,4-dihydro-1H-isoquinolin-5-yl)amino]pyrido[2,3-d]pyrimidin-7-one;
(Z)-1⁵-ethynyl-1⁶-methyl-1⁷,1⁸-dihydro-4-oxa-2-aza-1(2,8)-pyrido[2,3-d]pyrimidina-3(1,3)-benzenacyclooctaphan-6-en-1⁷-one;
1⁵-ethynyl-1⁶-methyl-1⁷,1⁸-dihydro-4-oxa-2-aza-1(2,8)-pyrido[2,3-d]pyrimidina-3(1,3)-benzenacyclooctaphan-1⁷-one;
(5¹R,5³S)-3⁵-ethynyl-3⁷,3⁸-dihydro-7-oxa-2-aza-3(2,8)-pyrido[2,3-d]pyrimidina-1(1,3)-benzena-5(1,3)-cyclopentanacycloheptaphan-3⁷-one;
1⁵-Ethynyl-1⁷,1⁸-dihydro-4-oxa-2-aza-1(2,8)-pyrido[2,3-d]pyrimidina-3(1,3)-benzenacyclooctaphan-1⁷-one;
1⁵-Ethynyl-1⁶-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-1⁷-one;
(Z)-1⁵-ethynyl-1⁷,1⁸-dihydro-5-oxa-2-aza-1(2,8)-pyrido[2,3-d]pyrimidina-3(1,3)-benzenacyclononaphan-7-en-1⁷-one;
1⁵-Ethynyl-3⁴-(4-methylpiperazin-1-yl)-1⁷,1⁸-dihydro-5-oxa-2-aza-1(2,8)-pyrido[2,3-d]pyrimidina-3(1,3)-benzenacyclooctaphan-1⁷-one;
1⁵-Ethynyl-3⁴-(4-methylpiperazin-1-yl)-1⁷,1⁸-dihydro-2,6-diaza-1(2,8)-pyrido[2,3-d]pyrimidina-3(1,3)-benzenacyclooctaphane-1⁷,5-dione;
1⁵-Ethynyl-3⁴-(4-methylpiperazin-1-yl)-1⁷,1⁸-dihydro-2,4-diaza-1(2,8)-pyrido[2,3-d]pyrimidina-3(1,3)-benzenacyclooctaphan-1⁷-one;
1⁵-Ethynyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphan-1⁷-one;
1⁵-Ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclononaphan-17-one;
N-(5-((5-ethynyl-7-oxo-7,8-dihydropyrido[2,3-d]pyrimidin-2-yl)amino)-2-(4-methylpiperazin-1-yl)benzyl)acrylamide;
8-cyclopentyl-5-ethynyl-2-((2-(3-((methylsulfonyl)methyl)piperidin-1-yl)phenyl)amino)pyrido[2,3-d]pyrimidin-7(8H)-one;
8-cyclopentyl-5-ethynyl-2-((3-(methyl((1s,3s)-3-((methylsulfonyl)methyl)cyclobutyl)amino)phenyl)amino)pyrido[2,3-d]pyrimidin-7(8H)-one;
8-cyclopentyl-5-ethynyl-2-((3-(methyl((1s,4s)-4-((methylsulfonyl)methyl)cyclohexyl)amino)phenyl)amino)pyrido[2,3-d]pyrimidin-7(8H)-one;
8-cyclopentyl-5-ethynyl-2-((3-(methyl((1r,3r)-3-((methylsulfonyl)methyl)cyclobutyl)amino)phenyl)amino)pyrido[2,3-d]pyrimidin-7(8H)-one;
8-cyclopentyl-5-ethynyl-2-((3-(methyl((1r,4r)-4-((methylsulfonyl)methyl)cyclohexyl)amino)phenyl)amino)pyrido[2,3-d]pyrimidin-7(8H)-one;
5-ethynyl-8-methyl-2-((4-(4-methylpiperazin-1-yl)-3-propoxyphenyl)amino)pyrido[2,3-d]pyrimidin-7(8H)-one;
5-ethynyl-2-((3-methoxy-4-(4-methylpiperazin-1-yl)phenyl)amino)-8-methylpyrido[2,3-d]pyrimidin-7(8H)-one;
2-((3-(2-cyclopentylethoxy)-4-(4-methylpiperazin-1-yl)phenyl)amino)-5-ethynyl-8-methylpyrido[2,3-d]pyrimidin-7(8H)-one;
2-((3-(2-cyclopentylethoxy)-4-(4-methylpiperazin-1-yl)phenyl)amino)-5-ethynyl-8-((1-methyl-1H-pyrazol-3-yl)methyl)pyrido[2,3-d]pyrimidin-7(8H)-one;
2-((3-(cyclopentylmethoxy)-4-(4-methylpiperazin-1-yl)phenyl)amino)-5-ethynyl-8-methylpyrido[2,3-d]pyrimidin-7(8H)-one;
2-((3-(3-cyclopentylpropoxy)-4-(4-methylpiperazin-1-yl)phenyl)amino)-5-ethynyl-8-methylpyrido[2,3-d]pyrimidin-7(8H)-one;
2-((3-(2-cyclohexylethoxy)-4-(4-methylpiperazin-1-yl)phenyl)amino)-5-ethynyl-8-methylpyrido[2,3-d]pyrimidin-7(8H)-one;
5-ethynyl-8-methyl-2-((4-(4-methylpiperazin-1-yl)-3-phenethoxyphenyl)amino)pyrido[2,3-d]pyrimidin-7(8H)-one;
2-((3-(cyclopentyloxy)-4-(4-methylpiperazin-1-yl)phenyl)amino)-5-ethynyl-8-methylpyrido[2,3-d]pyrimidin-7(8H)-one;
5-ethynyl-2-((3-(isopentyloxy)-4-(4-methylpiperazin-1-yl)phenyl)amino)-8-methylpyrido[2,3-d]pyrimidin-7(8H)-one;
2-cyclopentyl-N-(5-((5-ethynyl-8-methyl-7-oxo-7,8-dihydropyrido[2,3-d]pyrimidin-2-yl)amino)-2-(4-methylpiperazin-1-yl)phenyl)acetamide;
N-(cyclopentylmethyl)-5-((5-ethynyl-8-methyl-7-oxo-7,8-dihydropyrido[2,3-d]pyrimidin-2-yl)amino)-2-(4-methylpiperazin-1-yl)benzenesulfonamide;
1-cyclopentyl-3-(5-((5-ethynyl-8-methyl-7-oxo-7,8-dihydropyrido[2,3-d]pyrimidin-2-yl)amino)-2-(4-methylpiperazin-1-yl)phenyl)urea;
5-ethynyl-8-methyl-2-((3-(2-(1-methyl-1H-pyrazol-3-yl)ethoxy)-4-(4-methylpiperazin-1-yl)phenyl)amino)pyrido[2,3-d]pyrimidin-7(8H)-one;
N-(cyclopentylmethyl)-5-((5-ethynyl-8-methyl-7-oxo-7,8-dihydropyrido[2,3-d]pyrimidin-2-yl)amino)-2-(4-methylpiperazin-1-yl)benzamide;
1⁵-ethynyl-3⁴-(4-methylpiperazin-1-yl)-1⁷,1⁸-dihydro-2,4-diaza-1(2,8)-pyrido[2,3-d]pyrimidina-3(1,3)-benzenacyclooctaphane-1⁷,5-dione;
1⁵-ethynyl-5-methyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphane-1⁷,6-dione;
1⁵-ethynyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphane-1⁷,4-dione;
1⁵-ethynyl-4-methyl-3⁴-(4-methylpiperazin-1-yl)-1⁷,1⁸-dihydro-2,4-diaza-1(2,8)-pyrido[2,3-d]pyrimidina-3(1,3)-benzenacyclooctaphane-1⁷,5-dione;
1⁵-ethynyl-5-methyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphan-1⁷-one;
1⁵-ethynyl-3⁴-(4-methylpiperazin-1-yl)-1⁷,1⁸-dihydro-2,6-diaza-1(2,8)-pyrido[2,3-d]pyrimidina-3(1,3)-benzenacyclooctaphane-1⁷,7-dione;
1⁵-ethynyl-9-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclononaphan-1⁷-one;
1⁵-ethynyl-16-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclononaphan-1⁷-one;
1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclodecaphan-1⁷-one;
1⁵-ethynyl-3⁴-(4-methylpiperazin-1-yl)-1⁷,1⁸-dihydro-5-oxa-2-aza-1(2,8)-pyrido[2,3-d]pyrimidina-3(1,3)-benzenacyclononaphan-1⁷-one;
1⁵-ethynyl-3⁴-(4-methylpiperazin-1-yl)-1⁷,1⁸-dihydro-4,7-dioxa-2-aza-1(2,8)-pyrido[2,3-d]pyrimidina-3(1,3)-benzenacyclononaphan-1⁷-one;
(5¹S,5³R)-3⁵-ethynyl-1⁴-(4-methylpiperazin-1-yl)-3⁷,3⁸-dihydro-7-oxa-2-aza-3(2,8)-pyrido[2,3-d]pyrimidina-1(1,3)-benzena-5(1,3)-cyclopentanacycloheptaphan-3⁷⁻one;
1⁵-ethynyl-3⁴-(4-methylpiperazin-1-yl)-1⁷,1⁸-dihydro-4-thia-2,5-diaza-1(2,8)-pyrido[2,3-d]pyrimidina-3(1,3)-benzenacyclooctaphan-1⁷-one 4,4-dioxide;
1⁵-ethynyl-3⁴-(4-isopropylpiperazin-1-yl)-1⁷,1⁸-dihydro-2,4-diaza-1(2,8)-pyrido[2,3-d]pyrimidina-3(1,3)-benzenacyclooctaphane-1⁷,5-dione;
1⁵-ethynyl-3⁴-(4-(2-methoxyethyl)piperazin-1-yl)-1⁷,1⁸-dihydro-2,4-diaza-1(2,8)-pyrido[2,3-d]pyrimidina-3(1,3)-benzenacyclooctaphane-1⁷,5-dione;
1⁵-ethynyl-3⁴-(4-methylpiperazin-1-yl)-1⁷,1⁸-dihydro-2,5-diaza-1(2,8)-pyrido[2,3-d]pyrimidina-3(1,3)-benzenacyclononaphane-1⁷,6-dione;
1⁵-ethynyl-3⁴-(4-methyl-1,4-diazepan-1-yl)-1⁷,1⁸-dihydro-2,4-diaza-1(2,8)-pyrido[2,3-d]pyrimidina-3(1,3)-benzenacyclooctaphane-1⁷,5-dione;
(R)-1⁵-ethynyl-8-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-1⁷-one;
1⁵-ethynyl-3⁴-(4-methylpiperazin-1-yl)-1⁷,1⁸-dihydro-6-oxa-2-aza-1(2,8)-pyrido[2,3-d]pyrimidina-3(1,3)-benzenacyclooctaphan-1⁷-one; and
1⁵-ethynyl-3⁴-(4-(2-methoxyethyl)piperazin-1-yl)-1⁷,1⁸-dihydro-4-oxa-2-aza-1(2,8)-pyrido[2,3-d]pyrimidina-3(1,3)-benzenacyclooctaphan-1⁷-one.

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Salts and Solvates

The compounds (including final products and intermediates) described herein may be isolated and used per se or may be isolated in the form of a salt, suitably pharmaceutically acceptable salts. It should be understood that the terms “salt(s)” and “salt form(s)” used by themselves or in conjunction with another term or terms encompasses all inorganic and organic salts, including industrially acceptable salts, as defined herein, and pharmaceutically acceptable salts, as defined herein, unless otherwise specified. As used herein, industrially acceptable salts are salts that are generally suitable for manufacturing and/or processing (including purification) as well as for shipping and storage, but may not be salts that are typically administered for clinical or therapeutic use. Industrially acceptable salts may be prepared on a laboratory scale, i.e. multi-gram or smaller, or on a larger scale, i.e. up to and including a kilogram or more.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in humans. A practitioner of ordinary skill will readily appreciate that some salts are both industrially acceptable as well as pharmaceutically acceptable salts. It should be understood that all such salts, including mixed salt forms, are within the scope of the application.

In one embodiment, the compounds of Formula I and sub-formulae thereof are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as, but not limited to, sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

Certain compounds of the Formula I and sub-formulae thereof may exist in solvated as well as unsolvated forms such as, for example, hydrated forms. It is to be understood that the invention encompasses all such solvated forms that possess the biological activity described herein.

### Polymorphs

It is also to be understood that certain compounds of the Formula I and sub-formulae thereof may exhibit polymorphism, and that the invention encompasses all such forms that possess the biological activity described herein.

### N-Oxides

Compounds of the Formula I and sub-formulae thereof containing an amine function may also form N-oxides. A reference herein to a compound of the Formula I and sub-formulae thereof that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as, but not limited to, hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as, but not limited to, dichloromethane.

### Tautomers

Compounds of the Formula I and sub-formulae thereof may exist in a number of different tautomeric forms and references to compounds of the Formula I and sub-formulae thereof include all such forms. For the avoidance of doubt, where a compound can exist in one of several tautomeric forms, and only one is specifically described or shown, all others are nevertheless embraced by Formula I and sub-formulae thereof. Examples of tautomeric forms include keto-, enol-, and enolate-forms, as in, for example, the following tautomeric pairs: keto/enol (illustrated below), pyrimidone/hydroxypyrimidine, imine/enamine, amide/imino alcohol, amidine/amidine, nitroso/oxime, thioketone/enethiol, and nitro/aci-nitro.

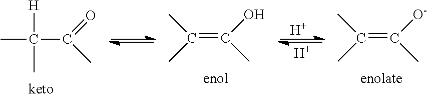

### Isomers

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Certain compounds of Formula I and sub-formulae thereof may have one or more asymmetric centres and therefore can exist in a number of stereoisomeric configurations. Consequently, such compounds can be synthesized and/or isolated as mixtures of enantiomers and/or as individual (pure) enantiomers, and, in the case of two or more asymmetric centres, single diastereomers and/or mixtures of diastereomers. It should be understood that the present application includes all such enantiomers and diastereomers and mixtures thereof in all ratios.

### Isotopes

The compounds of the present invention are described herein using structural formulas that do not specifically recite the mass numbers or the isotope ratios of the constituent atoms. As such it is intended that the present application includes compounds in which the constituent atoms are present in any ratio of isotope forms. For example, carbon atoms may be present in any ratio of ¹²C, ¹³C, and ¹⁴C; hydrogen atoms may be present in any ratio of ¹H, ²H, and ³H; etc. Preferably, the constituent atoms in the compounds of the present invention are present in their naturally occurring ratios of isotope forms.

### Prodrugs and Metabolites

The compounds of Formula I and sub-formulae thereof may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and in-vivo cleavable amide derivatives that may be formed at a carboxy group or an amino group in a compound of the Formula I and sub-formulae thereof.

Accordingly, the present invention includes those compounds of the Formula I and sub-formulae thereof as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I and sub-formulae thereof may be a synthetically-produced compound or a metabolically-produced compound.

A suitable pharmaceutically acceptable pro-drug of a compound of the Formula I and sub-formulae thereof is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula I and sub-formulae thereof that possesses a carboxy group is, for example, an in vivo cleavable ester thereof. An in vivo cleavable ester of a compound of the Formula I containing a carboxy group is, for example, a pharmaceutically acceptable ester which is cleaved in the human or animal body to produce the parent acid. Suitable pharmaceutically acceptable esters for carboxy include C₁₋₆alkyl esters such as, but not limited to, methyl, ethyl and tert-butyl, C₁₋₆alkoxymethyl esters such as, but not limited to, methoxymethyl esters, C₁₋₆alkanoyloxymethyl esters such as, but not limited to, pivaloyloxymethyl esters, 3-phthalidyl esters, C₃₋₆cycloalkylcarbonyloxy-C₁₋₆alkyl esters such as, but not limited to, cyclopentylcarbonyloxymethyl and 1-cyclohexylcarbonyloxyethyl esters, 2-oxo-1,3-dioxolenylmethyl esters such as, but not limited to, 5-methyl-2-oxo-1,3-dioxolen-4-ylmethyl esters and C₁₋₆alkoxycarbonyloxy-C₁₋₆alkyl esters such as, but not limited to, methoxycarbonyloxymethyl and 1-methoxycarbonyloxyethyl esters.

A suitable pharmaceutically acceptable pro-drug of a compound of the Formula I and sub-formulae thereof that possesses a hydroxy group is, for example, an in vivo cleavable ester or ether thereof. An in vivo cleavable ester or ether of a compound of the Formula I and sub-formulae thereof containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as, but not limited to, phosphate esters (including phosphoramidic cyclic esters). Further suitable pharmaceutically acceptable ester forming groups for a hydroxy group include C₁₋₁₀alkanoyl groups such as, but not limited to, acetyl, benzoyl, phenylacetyl and substituted benzoyl and phenylacetyl groups, C₁₋₁₀alkoxycarbonyl groups such as, but not limited to, ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as, but not limited to, acetoxymethyl and pivaloyloxymethyl groups.

A suitable pharmaceutically acceptable pro-drug of a compound of the Formula I and sub-formulae thereof that possesses a carboxy group is, for example, an in vivo cleavable amide thereof, for example an amide formed with an amine such as, but not limited to, ammonia, a C₁₋₄alkylamine such as, but not limited to, methylamine, a (C₁₋₄alkyl)₂amine such as, but not limited to, dimethylamine, N-ethyl-N-methylamine or diethylamine, a C₁₋₄alkoxy-C₂₋₄alkylamine such as, but not limited to, 2-methoxyethylamine, a phenyl-C₁₋₄ alkylamine such as, but not limited to, benzylamine and amino acids such as, but not limited to, glycine or an ester thereof.

A suitable pharmaceutically acceptable pro-drug of a compound of the Formula I and sub-formulae thereof that possesses an amino group is, for example, an in vivo cleavable amide derivative thereof. Suitable pharmaceutically acceptable amides from an amino group include, for example an amide formed with C₁₋₁₀alkanoyl groups such as, but not limited to, an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the Formula I and sub-formulae thereof may be exerted in part by one or more metabolites that are formed within the human or animal body after administration of a compound of the Formula I and sub-formulae thereof. As stated hereinbefore, the in vivo effects of a compound of the Formula I and sub-formulae thereof may also be exerted by way of metabolism of a precursor compound (a pro-drug).

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 1.5 g of active agent (more suitably from 0.5 to 600 mg, for example from 1 to 200 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the conditions, the age and sex of the animal or patient and the route of administration, according to well-known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used.

For the compounds of the present invention, oral administration is particularly suitable. The compounds of the present invention may be formulated as a tablet, capsule or solution for oral administration. Suitably, the compound of the present invention is formulated in a unit dosage form (e.g. a tablet or capsule) for oral administration. Typically, unit dosage forms will contain about 0.5 mg to 1.5 g of a compound of this invention.

### Synthesis

The compounds of the present invention can be prepared by any suitable technique known in the art. Particular methods for forming compounds of formula I defined herein are shown below and in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For Examples of protecting groups see one of the many general texts on the subject, for example, ‘Protective Groups in Organic Synthesis' by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as, but not limited to, acetyl, an alkoxycarbonyl group, for example a methoxycarbonyl, ethoxycarbonyl or butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as, but not limited to, an alkali metal hydroxide, for example lithium or sodium hydroxide. Alternatively an acyl group such as a tertbutoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium on carbon.

Resins may also be used as a protecting group.

The methodology employed to synthesise a compound of formula (I) will vary depending on the nature of R_{N}, R₁, V₁, and Q and any substituent groups associated therewith. Suitable processes for their preparation are described further in the accompanying Examples.

Once a compound of formula (I) has been synthesised by any one of the processes defined herein, the processes may then further comprise one or more of the additional steps of:
(i) removing any residual protecting groups present;
(ii) converting the compound formula (I) into another compound of formula (I);
(iii) forming a pharmaceutically acceptable salt, hydrate or solvate of the compound of formula I; and/or
(iv) forming a prodrug of the compound of formula I.

An Example of (ii) above is when a compound of formula (I) is synthesised and then one or more of the groups of R_{N}, R₁, V₁, and Q may be further reacted to change the nature of the group and provide an alternative compound of formula (I).

The resultant compounds of formula (I) can be isolated and purified using techniques well known in the art.

### Therapeutic Uses and Applications

The compounds of the present invention are inhibitors of EGFR, including mutated forms of EGFR that are resistant to third generations EGFR inhibitors, such as osimertinib and lazertinib. Data showing the EGFR inhibitory activity of the exemplified compounds is presented in the accompanying example section.

Accordingly, the compounds of formula I are useful for the treatment and/or prevention of diseases and conditions in which EGFR activity, including mutated forms of EGFR₁ is implicated, such as, for example, but not limited to, the treatment and/or prevention of proliferative disorders (e.g. cancer).

Therefore, in one aspect, the present invention provides a compound of Formula I as defined herein, or a pharmaceutically acceptable salt, hydrate or solvate thereof, or a pharmaceutical composition as defined herein, for use in therapy.

In another aspect, the present invention provides a compound of Formula I as defined herein, or a pharmaceutically acceptable salt, hydrate or solvate thereof, or a pharmaceutical composition as defined herein, for use in the treatment of a disease or condition in which EGFR activity is implicated.

In another aspect, the present invention provides a compound of Formula I as defined herein, or a pharmaceutically acceptable salt, hydrate or solvate thereof, or a pharmaceutical composition as defined herein, for use in the treatment of a disease or condition associated with aberrant activity of EGFR.

In another aspect, the present invention provides a compound of Formula I as defined herein, or a pharmaceutically acceptable salt, hydrate or solvate thereof, or a pharmaceutical composition as defined herein, for use in the treatment of proliferative disorders (e.g. cancer).

In another aspect, the present invention provides a compound of Formula I as defined herein, or a pharmaceutically acceptable salt, hydrate or solvate thereof, or a pharmaceutical composition as defined herein, for use in the treatment of cancer.

In another aspect, the present invention provides the use of a compound of Formula I as defined herein, or a pharmaceutically acceptable salt, hydrate or solvate thereof, in the manufacture of a medicament for use in the treatment of a disease or condition in which EGFR activity is implicated.

In another aspect, the present invention provides the use of a compound of Formula I as defined herein, or a pharmaceutically acceptable salt, hydrate or solvate thereof, in the manufacture of a medicament for use in the treatment of a disease or condition associated with aberrant activity of EGFR.

In another aspect, the present invention provides the use of a compound of Formula I as defined herein, or a pharmaceutically acceptable salt, hydrate or solvate thereof, in the manufacture of a medicament for use in the treatment of proliferative disorders (e.g. cancer or benign neoplasms).

In another aspect, the present invention the use of a compound of Formula I as defined herein, or a pharmaceutically acceptable salt, hydrate or solvate thereof, in the manufacture of a medicament for use in the treatment of a cancer.

In another aspect, the present invention provides a method of treating a disease or condition in which EGFR activity is implicated, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a disease or condition associated with aberrant activity of EGFR, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a proliferative disorder (e.g. cancer or benign neoplasms), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of formula I, or a pharmaceutically acceptable salt, hydrate or solvate thereof, for use in the treatment of an EGFR positive cancer, optionally selected from head and neck cancer, brain cancer, breast cancer, colon cancer and/or lung cancer.

In another aspect, the present invention provides a compound of formula I, or a pharmaceutically acceptable salt, hydrate or solvate thereof, for use in the treatment of EGFR positive non-small cell lung cancer.

In another aspect, the present invention provides a compound of formula I, or a pharmaceutically acceptable salt, hydrate or solvate thereof, for use in the treatment of a cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides a compound of formula I or a pharmaceutically acceptable salt, hydrate or solvate thereof, for use in the treatment of non-small cell lung cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides a compound of formula I, or a pharmaceutically acceptable salt, hydrate or solvate thereof, for use in the treatment of a cancer resistant to treatment with a third generation EFGR inhibitor, e.g. osimertinib, lazertinib (YH25448), EGF816, olmutinib, PF-06747775, avitinib and/or rociletinib.

In another aspect, the present invention provides a compound of formula I, or a pharmaceutically acceptable salt, hydrate or solvate thereof, for use in the treatment of non-small cell lung cancer resistant to treatment with osimertinib.

In another aspect, the present invention provides the use of a compound of formula I, or a pharmaceutically acceptable salt, hydrate or solvate thereof, in the manufacture of a medicament for use in the treatment of an EGFR positive cancer, optionally selected from head and neck cancer, brain cancer, breast cancer, colon cancer and/or lung cancer.

In another aspect, the present invention provides the use of a compound of formula I, or a pharmaceutically acceptable salt, hydrate or solvate thereof, in the manufacture of a medicament for use in the treatment of EGFR positive non-small cell lung cancer.

In another aspect, the present invention provides the use of a compound of formula I, or a pharmaceutically acceptable salt, hydrate or solvate thereof, in the manufacture of a medicament for use in the treatment of a cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides the use of a compound of formula I or a pharmaceutically acceptable salt, hydrate or solvate thereof, in the manufacture of a medicament for use in the treatment of non-small cell lung cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides the use of a compound of formula I, or a pharmaceutically acceptable salt, hydrate or solvate thereof, in the manufacture of a medicament for use in the treatment of a cancer resistant to treatment with a third generation EFGR inhibitor, e.g. osimertinib, lazertinib (YH25448), EGF816, olmutinib, PF-06747775, avitinib and/or rociletinib.

In another aspect, the present invention provides the use of a compound of formula I, or a pharmaceutically acceptable salt, hydrate or solvate thereof, in the manufacture of a medicament for use in the treatment of non-small cell lung cancer resistant to treatment with osimertinib.

In another aspect, the present invention provides a method of treating an EGFR positive cancer, optionally selected from head and neck cancer, brain cancer, breast cancer, colon cancer and/or lung cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating EGFR positive non-small cell lung cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a cancer expressing a mutated form of EGFR (for example EGFR comprising a T790M mutation, a deletion in exon 19 (such as A740-A750), an exon 20 insertion, a mutation at L858R and/or a C797S mutation), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating non-small cell lung cancer expressing a mutated form of EGFR (for example EGFR comprising a T790M mutation, a deletion in exon 19 (such as A740-A750), an exon 20 insertion, a mutation at L858R and/or a C797S mutation), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a cancer resistant to treatment with a third generation EFGR inhibitor, e.g. osimertinib, lazertinib (YH25448), EGF816, olmutinib, PF-06747775, avitinib and/or rociletinib, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating non-small cell lung cancer resistant to treatment with osimertinib, said method comprising administering to a subject in need thereof an effective amount of a compound of Formula I as defined herein, or a pharmaceutically acceptable salt, hydrate or solvate thereof, or a pharmaceutical composition as defined herein.

The terms “proliferative disorder” and “proliferative condition” are used interchangeably herein and pertain to an unwanted or uncontrolled cellular proliferation of excessive or abnormal cells which is undesired, such as, neoplastic or hyperplastic growth, whether in vitro or in vivo.

Examples of proliferative conditions include, but are not limited to, pre-malignant and malignant cellular proliferation, including but not limited to, cancers, psoriasis, bone diseases, fibroproliferative disorders (e.g. of connective tissues), and atherosclerosis. Any type of cell may be treated.

Cancers associated with aberrant EGFR activity (including its mutated forms—for example EGFR comprising a T790M mutation, a deletion in exon 19 (such as A740-A750), an exon 20 insertion, a mutation at L858R and/or a C797S mutation) are of particular interest. Thus, the compounds of the present invention may be used to treat any EGFR positive cancer. Particular examples of such cancers include head and neck cancer, brain cancer, breast cancer, colon cancer and/or lung cancer. A particular cancer of interest is non-small cell lung cancer.

Thus, in certain aspects of the present invention, the proliferative disorder is cancer, suitably a cancer selected from head and neck cancer, brain cancer, breast cancer, colon cancer and/or lung cancer.

In a particular aspect of the invention, the proliferative disorder is non-small cell lung cancer.

It will be appreciated that the compounds of the present invention could be used to treat any EGFR positive cancer. The invention therefore encompasses the treatment of any EGFR positive non-metastatic or metastatic cancer and which may be a solid tumour or a haematological (“liquid”) cancer. The cancer may, for example, be selected from:
(1) Carcinoma, including for example tumours derived from stratified squamous epithelia (squamous cell carcinomas) and tumours arising within organs or glands (adenocarcinomas). Examples include breast, colon, lung, prostate, ovary, esophageal carcinoma (including, but not limited to, esophageal adenocarcinoma and squamous cell carcinoma), basal-like breast carcinoma, basal cell carcinoma (a form of skin cancer), squamous cell carcinoma (various tissues), head and neck carcinoma (including, but not limited to, squamous cell carcinomas), stomach carcinoma (including, but not limited to, stomach adenocarcinoma, gastrointestinal stromal tumor), signet ring cell carcinoma, bladder carcinoma (including transitional cell carcinoma (a malignant neoplasm of the bladder)), bronchogenic carcinoma, colorectal carcinoma (including, but not limited to, colon carcinoma and rectal carcinoma), anal carcinoma, gastric carcinoma, lung carcinoma (including but not limited to small cell carcinoma (SCLC) and non-small cell carcinoma of the lung (NSCLC), lung adenocarcinoma, squamous cell carcinoma, large cell carcinoma, bronchioloalveolar carcinoma, and mesothelioma), neuroendocrine tumors (including but not limited to carcinoids of the gastrointestinal tract, breast, and other organs), adrenocortical carcinoma, thyroid carcinoma, pancreatic carcinoma (including, but not limited to, pancreatic ductal adenocarcinoma, pancreatic adenocarcinoma, acinar cell carcinoma, intraductal papillary mucinous neoplasm with invasive carcinoma, mucinous cystic neoplasm with invasive carcinoma, islet cell carcinoma and neuroendocrine tumors), breast carcinoma (including, but not limited to, ductal carcinoma, lobular carcinoma, inflammatory breast cancer, clear cell carcinoma, mucinous carcinoma), ovarian carcinoma (including, but not limited to, ovarian epithelial carcinoma or surface epithelial-stromal tumor including serous tumor, endometrioid tumor and mucinous cystadenocarcinoma, sex-cord-stromal tumor), liver and bile duct carcinoma (including, but not limited to, hepatocellular carcinoma, cholangiocarcinoma and hemangioma), prostate carcinoma, adenocarcinoma, brain tumours (including, but not limited to glioma, glioblastoma and medulloblastoma), germ cell tumors, sweat gland carcinoma, sebaceous gland carcinoma, papillary carcinoma, papillary adenocarcinoma, cystadenocarcinoma, kidney carcinoma (including, but not limited to, renal cell carcinoma, clear cell carcinoma and Wilm's tumor), medullary carcinoma, ductal carcinoma in situ or bile duct carcinoma, choriocarcinoma, seminoma, embryonal carcinoma, cervical carcinoma, uterine carcinoma (including, but not limited to, endometrial adenocarcinoma, uterine papillary serous carcinoma, uterine clear-cell carcinoma, uterine sarcomas and leiomyosarcomas, mixed mullerian tumors), testicular carcinoma, osteogenic carcinoma, epithelial carcinoma, sarcomatoid carcinoma, nasopharyngeal carcinoma, laryngeal carcinoma; oral and oropharyngeal squamous carcinoma;
(2) Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and hemangioendothelioma (blood vessels); liposarcoma (adipose tissue); glioma and astrocytoma (neurogenic connective tissue found in the brain); myxosarcoma (primitive embryonic connective tissue); chordoma, endotheliosarcoma, lymphangiosarcoma, lymphangioendotheliosarcoma, synovioma, Ewing's sarcoma, mesenchymous and mixed mesodermal tumor (mixed connective tissue types) and other soft tissue sarcomas;
(3) Myeloma and multiple myeloma;
(4) Hematopoietic tumours, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating); myelofibrosis.
(5) Lymphomas, including: Hodgkin and Non-Hodgkin lymphomas;
(6) Solid tumors of the nervous system including medulloblastoma, craniopharyngioma, ependymoma, pinealoma, hemangioblastoma, acoustic neuroma, oligodendroglioma, meningioma, neuroblastoma and schwannoma;
(7) Melanoma, uveal melanoma and retinoblastoma; and
(8) Mixed Types, including, e.g., adenosquamous carcinoma, mixed mesodermal tumor, carcinosarcoma or teratocarcinoma.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (e.g. by a patch, plaster, etc.); transmucosal (e.g. by a patch, plaster, etc.); intranasal (e.g. by nasal spray); ocular (e.g. by eye drops, eye ointment etc.); pulmonary (e.g. by inhalation or insufflation therapy, for example via an aerosol, for example by the nose or mouth); rectal (e.g. by suppository or enema); vaginal (e.g. by pessary); parental, for example by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir dosage form, for example subcutaneously or intramuscularly.

The compounds of the present invention are particularly suitable for oral administration.

### Combination Therapies

The compounds of the invention and salts, solvates thereof defined hereinbefore may be applied as a sole therapy or may involve, in addition to the compound of the invention, one or more additional therapeutic agents, e.g. an anti-tumour agent.

In the context of cancer treatment, in addition to the compound of the invention therapy may involve conventional surgery or radiotherapy or chemotherapy. Such chemotherapy may include one or more of the following categories of anti-tumour agents:—
other antiproliferative/antineoplastic drugs and combinations thereof, as used in medical oncology, such as, but not limited to, alkylating agents (for example cisplatin, oxaliplatin, carboplatin, cyclophosphamide, nitrogen mustard, melphalan, chlorambucil, busulphan, temozolamide and nitrosoureas); antimetabolites (for example gemcitabine and antifolates such as, but not limited to, fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as, but not limited to, finasteride;
anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example other EGFR family tyrosine kinase inhibitors such as, but not limited to, N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (Cl 1033), afatinib, osirnertinib, lazertinib (YH25448), EGF816, olmutinib, PF-06747775, avitinib, rociletinib; erbB2 tyrosine kinase inhibitors such as, but not limited to, lapatinib); inhibitors of the hepatocyte growth factor family; inhibitors of the insulin growth factor family; inhibitors of the platelet-derived growth factor family such as, but not limited to, imatinib and/or nilotinib (AMN107); inhibitors of serine/threonine kinases (for example Ras/Raf signalling inhibitors such as, but not limited to, farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as, but not limited to, CDK2 and/or CDK4 inhibitors;
antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as, but not limited to, vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as, but not limited to, those disclosed in International Patent Applications WO97/22596, WO 97/30035, WO 97/32856 and WO 98/13354 and compounds that work by other mechanisms (for example linomide, inhibitors of integrin av33 function and angiostatin)];
vascular damaging agents such as, but not limited to, Combretastatin A4 and compounds disclosed in International Patent Applications WO 99/02166, WO 00/40529, WO 00/41669, WO 01/92224, WO 02/04434 and WO 02/08213;
an endothelin receptor antagonist, for example zibotentan (ZD4054) or atrasentan;
antisense therapies, for example those which are directed to the targets listed above, such as, but not limited to, ISIS 2503, an anti-ras antisense;
gene therapy approaches, including for example approaches to replace aberrant genes such as, but not limited to, aberrant p53 or aberrant BRCA1 or BRCA2, GDEPT (gene-directed enzyme pro-drug therapy) approaches such as, but not limited to, those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex-vivo and in-vivo approaches to increase the immunogenicity of patient tumour cells, such as, but not limited to, transfection with cytokines such as interleukin 2, interleukin 4 or granulocyte-macrophage colony stimulating factor, approaches to decrease T-cell anergy, approaches using transfected immune cells such as, but not limited to, cytokine-transfected dendritic cells, approaches using cytokine-transfected tumour cell lines and approaches using anti-idiotypic antibodies.

In a particular embodiment, the antiproliferative treatment defined hereinbefore may involve, in addition to the compound of the invention, conventional surgery or radiotherapy or chemotherapy.

In a further particular embodiment, the antiproliferative treatment defined hereinbefore may involve, in addition to the compound of the invention, standard chemotherapy for the cancer concerned.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt, hydrate or solvate thereof, and another anti-tumour agent, or a pharmaceutically acceptable salt thereof.

According to this aspect of the invention there is provided a combination for use in the treatment of a proliferative condition, such as, but not limited to, cancer (for example a cancer involving a solid tumour), comprising a compound of the invention as defined hereinbefore, or a pharmaceutically acceptable salt, hydrate or solvate thereof, and any one of the anti-tumour agents listed herein above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination. In one embodiment, a combination refers to a combination product.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### Biological Activity

The biological assays described in the example section may be used to measure the pharmacological effects of the compounds of the present invention. Although the pharmacological properties of the compounds of formula I vary with structural change, as expected, the compounds of the invention were found to be active in the assays described in the example section below.

### EXAMPLES

The invention will now be illustrated, but not limited, by reference to the specific embodiments described in the following examples. Compounds are named using conventional IUPAC nomenclature, or as named by the chemical supplier.

The following synthetic procedures are provided for illustration of the methods used; for a given preparation or step the precursor used may not necessarily derive from the individual batch synthesized according to the step in the description given.

### General Information

Chemicals were purchased from commercial suppliers and used without further purification. Thin layer chromatography (TLC) was performed on aluminium plates coated with 60 F254 silica from Merck. Flash chromatography was carried out using a Biotage SP4, Biotage Isolera, or Varian automated flash system with Silicycle or GraceResolve normal phase silica gel prepacked columns. Fractions were collected at 254 nm or if necessary, on all wavelengths between 200 and 400 nm. Microwave irradiation was performed in a Biotage Initiator Sixty in sealed vials. Reactions were irradiated at 2.45 GHz and were able to reach temperatures between 6° and 250° C. Heating was at a rate of 2-5° C./s, and the pressure was able to reach 20 bar. Final compound purity is >95%. Flash chromatography was also carried out using a Biotage SP4, Biotage Isolera Prime, Varian or Agela Technologies automated flash system with GraceResolve normal phase silica gel pre-packed columns, Welch Technology or Agela Technologies normal phase silica gel columns or reverse phase C18 columns. Fractions were collected at 254 nm or if necessary, on all wavelengths between 200 and 400 nm. Microwave irradiation was performed in a Biotage Initiator Sixty in sealed vials. Reactions were irradiated at 2.45 GHz and were able to reach temperatures between 4° and 300° C. Heating was at a rate of 2-5° C./s and the pressure was able to reach 20 bar.

### Analytical Equipment

LC-MS analyses were conducted using a Waters Acquity UPLC system or Shimadzu LCMS-2020 with photo diode array (PDA) and evaporating light scattering detector (ELSD). When a 2 min gradient was used, the sample was eluted on an Acquity UPLC BEH C18, 1.7 μm (2.1×50 mm) with a flow rate of 0.6 mL/min using 5-95% 0.1% HCOOH in MeCN or LCMS Ascentis Express 90 A C18 2.7 μm (3 cm×3.0 mm) with a flow rate of 1.2 mL/min using 5-95% 0.1% HCOOH in MeCN or Poroshell HPH—C18, 4.0 μm (5 cm×3.0 mm) with a flow rate of 1.2 mL/min using 5-95% 5 mmol NH₄HCO₃ in MeCN. The analytical purity of compounds was determined using Waters XTerra RP18, 5 μm (4.6×150 mm) column at 1 mL/min either using 0.1% aq. ammonia and MeCN or 0.1% aq. HCOOH and MeCN with a gradient of 5-100% over 15 min or Shimadzu Ascentis Express C18, 2.7 μm (4.6×100 mm) column or Agilent EVO C18, 2.6 μm (3.0×100 mm) column at 1 mL/min using either 0.05% aq. ammonia and MeCN or 0.1% aq. TFA and MeCN with a gradient of 5-100% over 15 min.

¹H NMR spectra were obtained using a Bruker Avance III 500 spectrometer using a frequency of 500 MHz, a Bruker BioSpin AG Avanace III HD using a frequency of 300 MHz or a Bruker BioSpin AG Avanace NEO using a frequency of 400 MHz. ¹³C spectra were acquired using the Bruker Avance Ill 500 spectrometer operating at a frequency of 126 MHz. The abbreviations for spin multiplicity are as follows: s=singlet; d=doublet; t=triplet; q=quartet; p=quintuplet; h=sextuplet; m=multiplet. Combinations of these abbreviations are employed to describe more complex splitting patterns (e.g., dd=doublet of doublets).

### General Procedures

### General Procedure 1:

To a stirred solution/mixture of ethyl 4-chloro-2-(methylsulfanyl)pyrimidine-5-carboxylate (1.0 eq.) in THF (300 mL) was added amine (1.5 eq.) dropwise at 0° C. under nitrogen. Once completed, the resulting mixture was filtered, the filter cake was washed with THF (2×50 mL).

General procedure 2:

A solution of ethyl 4-chloro-2-(methylthio)pyrimidine-5-carboxylate (1.0 eq.), triethylamine (2.5 eq.) and amine (1.0 eq.) in THF (0.4 M) was stirred overnight at room temperature. The reaction mixture was diluted with ethyl acetate (250 mL) and water (250 mL). The organic phase was separated and concentrated under reduced pressure.

### General Procedure 3:

A solution of ester (1.0 eq.) and sodium hydroxide (5 eq.) in THF (250 mL) and water (250 mL) was stirred at 50° C. overnight. The resulting mixture was concentrated under reduced pressure to remove most of THF. Then the pH value was adjusted to 3-4 with 4 M aqueous hydrochloric acid and either the precipitate was collected by filtration, washing with water or the aqueous solution was extracted with ethyl acetate (3×250 mL). The combined organic extracts were concentrated under reduced pressure.

### General Procedure 4:

To a stirred solution of carboxylic acid (1.0 eq.) and 1H-benzo[d][1,2,3]triazole (1.0 eq.) in dichloromethane (0.3 M) was added EDCl (1.0 eq.). The resulting mixture was stirred at room temperature for 4 h. The reaction mixture was quenched with water (100 mL), the organic phase was separated and concentrated to dryness under reduced pressure.

### General Procedure 5:

To a solution of ethyl acetate (2.7 eq.) in THF (0.2 M) was slowly added LiHMDS (1M in THF, 2.7 eq.) at −78° C. The mixture was stirred for another 30 min at −78° C., then a solution of dicarbonyl (1.0 eq.) in THF (0.2 M) was added to the solution at −78° C. The resulting mixture was warmed to room temperature and stirred overnight. The reaction was quenched by 1 M aqueous hydrochloric acid then the pH was adjusted to 2 with 6 M aqueous hydrochloric acid and extracted with dichloromethane. The organic layers were combined, dried (Na₂SO₄) and concentrated under reduced pressure.

### General Procedure 6:

A solution of oxopropanoate (1.0 eq.), N,N-diisopropylethylamine (10 eq.) and DBU (2.0 eq.) was stirred at 120° C. for 1 hour then cooled. The solvent was decanted and the residual thick brown oil was dissolved in water (20 mL). The aqueous solution was acidified to pH 2 with 4 M aqueous hydrochloric acid. The resulting solids were collected by vacuum filtration, washing with water and 40-60 petroleum ether then dried under vacuum.

### General Procedure 7:

To a stirred solution of pyrimidine (1.0 eq.) in DMF (0.25 M) at 0° C. was slowly added sodium hydride (5.0 eq.). After 15 minutes at room temperature, the solution was cooled to 0° C. and the acyl chloride (1.2 eq.) was slowly added. After 1 hour at room temperature, the reaction was quenched with ice-water (200 mL) and extracted with ethyl acetate (2×200 mL). The aqueous layer was acidified to pH 5 with 5% aqueous hydrochloric acid and the resulting precipitate was collected by vacuum filtration or the combined organic layers were dried (Na₂SO₄) and concentrated under reduced pressure.

### General Procedure 8:

To a stirred solution/mixture of pyrido[2,3-d]pyrimidine-5,7-dione (1.0 eq.) and triethylamine (2.0 eq.) in dichloromethane (0.5 M) was added triflic anhydride (1.5 eq.) dropwise at 0° C. under a nitrogen atmosphere. The resulting mixture was stirred for 2 h at room temperature. The resulting mixture was concentrated under reduced pressure.

### General Procedure 9:

A solution of triflate (1.0 eq.), bis(triphenylphosphine)palladium chloride (0.10 eq.), triisopropylsilylacetylene (2.0 eq.), copper(I) iodide (0.10 eq.), N,N-diisopropylethylamine (3.0 eq.) in DMF (0.2 M). The resulting mixture was stirred for 3 h at 80° C. under a nitrogen atmosphere. The resulting mixture was filtered, the filter cake was washed with dichloromethane (2×10 mL).

### General Procedure 10:

Methylthiol (1.0 eq.) and mCPBA (3.0 eq.) in dichloromethane (0.1 M) were stirred at room temperature for 1-2 hours. The reaction mixture was quenched with saturated aqueous sodium thiosulphate solution (20 mL) and extracted with dichloromethane (3×30 mL). The combined organic extracts were washed with saturated aqueous sodium thiosulfate solution (3×20 mL) and saturated aqueous sodium hydrogen carbonate solution (3×30 mL). The organic layer was dried (MgSO₄) and concentrated under reduced pressure. The crude material was carried into the subsequent step without further purification.

### General Procedure 11:

p-fluoronitrophenyl (1.0 eq.), amine (1.0-2.0 eq.) and potassium carbonate (1.5-2.0 eq.) in DMSO (0.6-1.0 M) was stirred at room temperature overnight. The reaction mixture was poured onto water (30 mL) and the resulting precipitate collected by vacuum filtration washing with water and dried in a vacuum over overnight, or extracted with dichloromethane (3×50 mL), the combined organic extracts dried (MgSO₄) and concentrated under reduced pressure.

### General Procedure 12:

To a solution of nitro (1.0 eq.) in ethanol, methanol (0.25 M) or ethanol:acetic acid (1:1, 0.25 M) was added palladium on carbon 5-10 wt. % (0.1 eq.). The reaction mixture was stirred at room temperature overnight under an atmosphere of hydrogen. The reaction mixture was filtered through celite and the filtrate was concentrated under reduced pressure.

### General Procedure 13:

Methylsulfonyl (1.0 eq.), aniline (1.0-1.5 eq.) and trifluoroacetic acid (1.0-1.5 eq.) in acetonitrile or n-butanol (0.1 M) were stirred at 80-110° C. overnight or for the specified time. The reaction mixture was concentrated under reduced pressure.

### General Procedure 14:

Methylsulfonyl (1.0 eq.), amine (1.5 eq.) and N,N-diisopropylethylamine (5.0 eq.) in DMSO (0.25 M) were stirred at 60° C. overnight. The reaction mixture was quenched with saturated aqueous ammonium chloride solution (30 mL) and extracted with dichloromethane (3×30 mL). The combined organic extracts were dried (MgSO₄) and concentrated under reduced pressure.

### General Procedure 15:

TIPS-protected alkyne (1.0 eq.) and potassium fluoride (1.0-20 eq.) in solvent (0.1 M) was stirred at the specified temperature until completion. The solvent was removed under reduced pressure.

### General Procedure 16:

TES-protected alkyne (1.0 eq.) and potassium carbonate (1.0 eq.) in methanol (0.1 M) was stirred at 40° C. overnight. The solvent was removed under reduced pressure. The residue was suspended in water and the resulting precipitate collected by vacuum filtration, washing with water.

### General Procedure 17:

To a solution of bromide (1.0 eq.) and boronic acid (1.5 eq.) in dioxane (0.2 M) and water (0.45 M) was added potassium carbonate (2.0 eq.) and 1,1′-Bis(diphenylphosphino)ferrocene)palladium(II) dichloride (0.1 eq.). The reaction was stirred for 3 h at 70° C. under a nitrogen atmosphere, then concentrated under reduced pressure.

### General Procedure 18:

Pyridone (1.0 eq.), alcohol (1.1 eq.), triphenylphosphine (1.5-2.0 eq.) and DEAD or DIAD (1.5-2.0 eq.) in solvent (0.5 M) were stirred at room temperature for 1 h. The reaction mixture was quenched with water (30 mL), extracted with dichloromethane (3×30 mL). The combined organic extracts were dried (MgSO₄) and concentrated under reduced pressure.

### General Procedure 19

To a stirred solution of triflate (1.0 eq.) and bis(triphenylphosphine)palladium (II) dichloride (0.05 eq.) in toluene (0.14 M) was added vinyl tributyltin (1.4 eq.). The reaction was stirred at 80° C. for 1-2 h, then concentrated under reduced pressure. The residue was dissolved in hexane and extracted with acetonitrile. The acetonitrile layer was separated and concentrated under reduced pressure.

### General Procedure 20

Hydroxyl (1.0 eq.), triphenylphosphine (4.0 eq.) and DIAD (4.0 eq.) in THF (0.024 M) were stirred at room temperature for 1-2 h. The reaction mixture was quenched with water (30 mL) and extracted with dichloromethane (3×30 mL). The combined organic extracts were dried (MgSO₄) and concentrated under reduced pressure.

### General Procedure 21

To a stirred solution of alcohol (1.0 eq.) and silyl chloride (1.0-1.1 eq.) in THF (0.5 M) was added sodium hydride (1.0-1.1 eq.). The reaction was stirred at room temperature overnight, then quenched with saturated aqueous ammonium chloride solution (30 mL) and extracted with dichloromethane (3×30 mL). The combined organic extracts were dried (MgSO₄) and concentrated under reduced pressure.

### General Procedure 22

Phenol (1.0 eq.), hydroxyl (1.1 eq.), triphenylphosphine (1.3 eq.) and DIAD (1.3 eq.) in THF (0.2 M) were stirred at room temperature overnight. The reaction mixture was quenched with water (30 mL), extracted with dichloromethane (3×30 mL). The combined organic extracted with dried (MgSO₄) and concentrated under reduced pressure.

### General Procedure 23

To a stirred solution of nitro (1.0 eq.) in methanol (0.4 M) was added iron (5.0 eq.) and saturated aqueous ammonium chloride solution (0.8 M). The reaction mixture was stirred at 60° C. for 2-4 h. The reaction mixture was filtered through celite, washing with dichloromethane and methanol. The filtrate was separated, with the aqueous layer extracted with dichloromethane (3×30 mL). The combined organic layers were dried (MgSO₄) and concentrated under reduced pressure.

### Example 1

### Ethyl 2-(methylsulfanyl)-4-(phenylamino)pyrimidine-5-carboxylate

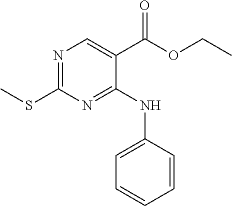 [SMILES: CCOC(=O)c1cnc(SC)nc1Nc1ccccc1]

General procedure 2 was ap GP-15 C1 ethyl 4-chloro-2-(methylsulfanyl)pyrimidine-5-carboxylate (100 g, 425 mmol), THF (1.0 L), triethylamine (129 g, 1.28 mol) and aniline (59.4 g, 638 mmol). The residue was slurried in 40-60 petroleum ether (100 mL) and filtered. The filter cake was washed with 40-60 petroleum ether and then dried under vacuum to yield the title compound as an off-white solid (115 g, 93%), which was used in next step without any further purification.

(ES, m/z): [M+H]+=289.9

### 2-(Methylsulfanyl)-4-(phenylamino)pyrimidine-5-carboxylic acid

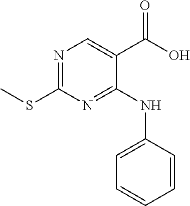 [SMILES: CSc1ncc(C(=O)O)c(Nc2ccccc2)n1]

General procedure 3 was applied to ethyl 2-(methylsulfanyl)-4-(phenylamino)pyrimidine-5-carboxylate (147 g, 507 mmol) and sodium hydroxide (71.0 g, 1.78 mol) in THF (1.5 L) and water (1.5 L) to afford the title compound as a white solid (110 g, 82%).

(ES, m/z): [M+H]⁺=261.9

### 5-(1,2,3-Benzotriazole-1-carbonyl)-2-(methylsulfanyl)-N-phenylpyrimidin-4-amine

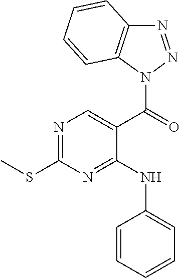 [SMILES: CSc1ncc(C(=O)n2nnc3ccccc32)c(Nc2ccccc2)n1]

General procedure 4 was applied to 2-(methylsulfanyl)-4-(phenylamino)pyrimidine-5-carboxylic acid (30.0 g) with EDCl (22.1 g) and benzotriazole (13.7 g) in dichloromethane (300 mL). The residue was purified by flash column chromatography eluting with ethyl acetate (10%) in 40-60 petroleum ether to yield the title compound as a yellow solid (35.6 g, 85% yield).

(ES, m/z): [M+H]+=363

### Ethyl 3-(2-(methylthio)-4-(phenylamino)pyrimidin-5-yl)-3-oxopropanoate

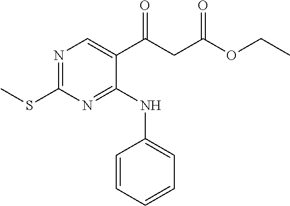 [SMILES: CCOC(=O)CC(=O)c1cnc(SC)nc1Nc1ccccc1]

General procedure 5 was applied to (1H-benzo[d][1,2,3]triazol-1-yl)(2-(methylthio)-4-(phenylamino)pyrimidin-5-yl)methanone (22.7 g, 62.7 mmol) with ethyl acetate (13.8 g, 157 mmol) and LiHMDS (1M, 157 mL, 157 mmol) in THF (550 mL) to yield the title compound as a light yellow solid (12.6 g, 61%).

(ES, m/z): [M+H]+=332

### 5-Hydroxy-2-(methylthio)-8-phenylpyrido[2,3-d]pyrimidin-7(8H)-one

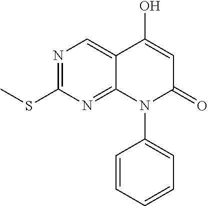 [SMILES: CSc1ncc2c(O)cc(=O)n(-c3ccccc3)c2n1]

General procedure 6 was applied to ethyl 3-(2-(methylthio)-4-(phenylamino)pyrimidin-5-yl)-3-oxopropanoate (12.6 g, 38.1 mmol) with N,N-diisopropylethylamine (51.0 mL, 29.2 mmol) and DBU (6.50 mL, 43.5 mmol) to yield the title compound as a as light brown solid (10.5 g, 97%).

(ES, m/z): [M+H]=286.

### 2-(Methylthio)-7-oxo-8-phenyl-7,8-dihydropyrido[2,3-d]pyrimidin-5-yl trifluoromethanesulfonate

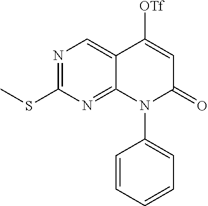 [SMILES: CSc1ncc2c(OS(=O)(=O)C(F)(F)F)cc(=O)n(-c3ccccc3)c2n1]

General procedure 8 was applied to 5-hydroxy-2-(methylthio)-8-phenylpyrido[2,3-d]pyrimidin-7(8H)-one (10.9 g, 38.3 mmol) with trifluoromethanesulfonic anhydride (16.2 g, 57.5 mmol) and triethylamine (5.82 g, 57.5 mmol) in dichloromethane (220 mL). The crude material was purified by flash column chromatography eluting with ethyl acetate (0-50%) in 40-60 petroleum ether to yield the title compound as a light yellow solid (8.80 g, 55%).

(ES, m/z): [M+H]*=418.

### 2-(Methylthio)-8-phenyl-5-((triisopropylsilyl)ethynyl)pyrido[2,3-d]pyrimidin-7(8H)-one

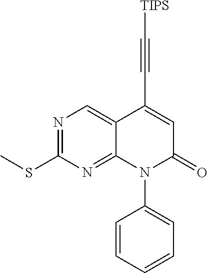

General procedure 9 was applied to 2-(methylthio)-7-oxo-8-phenyl-7,8-dihydropyrido[2,3-d]pyrimidin-5-yl trifluoromethanesulfonate (2.00 g, 4.80 mmol) with triisopropylsilyl acetylene (1.75 g, 9.60 mmol), copper(I) iodide (92.0 mg, 0.480 mmol), bis(triphenylphosphine)palladium(II) dichloride (338 mg, 0.480 mmol) in DMF (20 mL) and N,N-diisopropylethylamine (10 mL). The crude material was purified by reverse phase flash column chromatography eluting with acetonitrile (25-100%) in water (0.1% NH₄HCO₃) to yield the title compound as a light yellow solid (1.71 g, 80%).

(ES, m/z): [M+H]+=450.

### 2-(Methylsulfonyl)-8-phenyl-5-((triisopropylsilyl)ethynyl)pyrido[2,3-d]pyrimidin-7(8H)-one

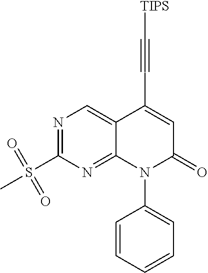

General procedure 10 was applied to 2-(methylthio)-8-phenyl-5-((triisopropylsilyl)ethynyl)pyrido[2,3-d]pyrimidin-7(8H)-one (1.70 g, 3.78 mmol) with m-CPBA (1.96 g, 11.4 mmol) in dichloromethane (90 mL). The crude material was purified by reverse phase flash column chromatography eluting with acetonitrile (25-100%) in water (0.1% NH₄HCO₃) to yield the title compound as a yellow solid (1.62 g, 89%).

(ES, m/z): [M+H]+=482.

### 2-((2-Methoxyphenyl)amino)-8-phenyl-5-((triisopropylsilyl)ethynyl)pyrido[2,3-d]pyrimidin-7(8H)-one

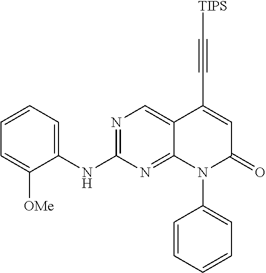

General procedure 13 was applied to 2-(methylsulfonyl)-8-phenyl-5-((triisopropylsilyl)ethynyl)pyrido[2,3-d]pyrimidin-7(8H)-one (1.32 g, 2.74 mmol) with 2-methoxyaniline (338 mg, 2.74 mmol) and trifluoroacetic acid (313 mg, 2.74 mmol) in 2-butanol (10 mL). The reaction mixture was stirred at 110° C. for 16 h. The crude material was purified by reverse phase flash column chromatography eluting with acetonitrile (25-100%) in water (0.1% NH₄HCO₃) to yield the title compound as a yellow solid (1.06 g, 74%).

(ES, m/z): [M+H]+=525.

### 5-Ethynyl-2-[(2-methoxyphenyl)amino]-8-phenylpyrido[2,3-d]pyrimidin-7-one

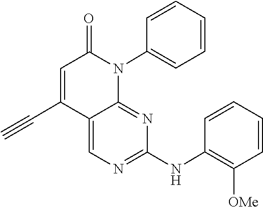 [SMILES: C#Cc1cc(=O)n(-c2ccccc2)c2nc(Nc3ccccc3OC)ncc12]

General procedure 15 was applied to 2-[(2-methoxyphenyl)amino]-8-phenyl-5-[2-(triisopropylsilyl)ethynyl]pyrido[2,3-d]pyrimidin-7-one (823 mg, 1.56 mmol), potassium fluoride (9.12 g, 156 mmol) in THF and water (22.0 mL, 10:1). The resulting solution was stirred for 48 h at 40° C. The crude material was purified by reverse phase flash column chromatography eluting with acetonitrile (25-100%) in water (0.1% NH₄HCO₃) to yield the title compound as a yellow solid (520 mg, 90%).

(ES, m/z): [M+H]⁺=369.

¹H NMR (300 MHz, CDCl₃) δ 8.91 (s, 1H), 8.10 (s, 1H), 7.76-7.49 (m, 4H), 7.36-7.30 (m, 2H), 6.98-6.86 (m, 1H), 6.86-6.73 (m, 2H), 6.52 (s, 1H), 3.88 (s, 3H), 3.71 (s, 1H).

### Example 2

### 5-(Bis(4-methoxybenzyl)amino)-2-(methylthio)-8-phenylpyrido[2,3-d]pyrimidin-7(8H)-one

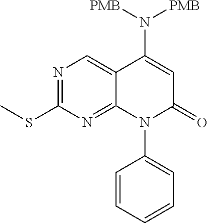

A solution of 2-(methylthio)-7-oxo-8-phenyl-7,8-dihydropyrido[2,3-d]pyrimidin-5-yl trifluoromethanesulfonate (1.00 g, 2.40 mmol, 1.0 eq.), bis(4-methoxybenzyl)amine (1.85 g, 7.20 mmol, 3.0 eq.) in DCE (15 mL) was heated to 100° C. under microwave irradiation for 6 h. After cooling to room temperature, the reaction mixture was concentrated under reduced pressure. The crude material was purified by reverse phase flash column chromatography eluting with acetonitrile (25-100%) in water (0.1% NH₄HCO₃) to yield the title compound as a white solid (1.80 g, 72%).

(ES, m/z): [M+H]⁺=525.

### 5-Amino-2-(methylthio)-8-phenylpyrido[2,3-d]pyrimidin-7(8H)-one

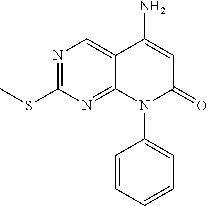 [SMILES: CSc1ncc2c(N)cc(=O)n(-c3ccccc3)c2n1]

A solution of 5-(bis(4-methoxybenzyl)amino)-2-(methylthio)-8-phenylpyrido[2,3-d]pyrimidin-7(8H)-one (1.80 g, 3.44 mmol, 1.0 eq.) in trifluoroacetic acid (20 mL) was stirred at 60° C. for 1 h. The reaction mixture was concentrated under reduced pressure and the residue dissolved in DMSO (5 mL). The DMSO solution was adjusted to neutral pH with triethylamine and purified by reverse phase flash column chromatography eluting with acetonitrile (15-100%) in water (0.1% NH₄HCO₃) to yield the title compound as a white solid (700 mg, 71%).

(ES, m/z): [M+H]⁺=285.

### tert-Butyl (2-(methylthio)-7-oxo-8-phenyl-7,8-dihydropyrido[2,3-d]pyrimidin-5-yl)carbamate

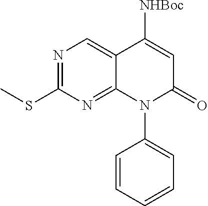 [SMILES: CSc1ncc2c(NC(=O)OC(C)(C)C)cc(=O)n(-c3ccccc3)c2n1]

To a stirred solution of 5-amino-2-(methylthio)-8-phenylpyrido[2,3-d]pyrimidin-7(8H)-one (700 mg, 2.46 mmol, 1.0 eq.), Boc anhydride (808 mg, 3.70 mmol, 1.5 eq.) and DMAP (30.0 mg, 0.246 mmol, 0.1 eq.) in dichloromethane (10 mL) at 0° C. was added triethylamine (497 mg, 4.92 mmol, 2.0 eq.) dropwise. The reaction was stirred at room temperature for 1 h before being diluted with water (25 mL) and dichloromethane (20 mL). The organic layer was separated, dried (Na₂SO₄) and concentrated under reduced pressure. The residue was purified by flash column chromatography eluting with ethyl acetate (0-50%) in 40-60 petroleum ether to yield a mixture of the title compound and the doubly protected product as a yellow solid (1.00 g, 100%).

(ES, m/z): [M+H]⁺=385.

### tert-Butyl (2-(methylsulfonyl)-7-oxo-8-phenyl-7,8-dihydropyrido[2,3-d]pyrimidin-5-yl)carbamate

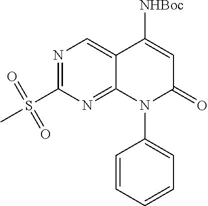 [SMILES: CC(C)(C)OC(=O)Nc1cc(=O)n(-c2ccccc2)c2nc(S(C)(=O)=O)ncc12]

General procedure 10 was applied to tert-butyl (2-(methylthio)-7-oxo-8-phenyl-7,8-dihydropyrido[2,3-d]pyrimidin-5-yl)carbamate (1.00 g, 2.46 mmol) and m-CPBA (1.50 g, 7.38 mmol) in dichloromethane (30 mL). The residue was purified by reverse phase flash column chromatography eluting with acetonitrile (25-100%) in water (0.1% NH₄HCO₃) to yield the title compound as a yellow solid (800 mg, 78%).

(ES, m/z): [M+H]⁺=417.

### Tert-Butyl (2-((2-methoxyphenyl)amino)-7-oxo-8-phenyl-7,8-dihydropyrido[2,3-d]pyrimidin-5-yl)carbamate

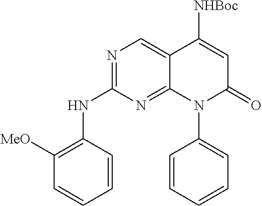 [SMILES: COc1ccccc1Nc1ncc2c(NC(=O)OC(C)(C)C)cc(=O)n(-c3ccccc3)c2n1]

General procedure 13 was applied to tert-butyl (2-(methylsulfonyl)-7-oxo-8-phenyl-7,8-dihydropyrido[2,3-d]pyrimidin-5-yl)carbamate (800 mg, 1.92 mmol), 2-methoxyaniline (236 mg, 1.92 mmol) and trifluoroacetic acid (186 mg, 1.92 mmol) and 2-butanol (10 mL). The reaction mixture was stirred at 110° C. for 16 h. The crude material was purified by reverse phase flash column chromatography eluting with acetonitrile (25-100%) in water (0.1% NH₄HCO₃) to yield the title compound as a yellow solid (500 mg, 57%).

(ES, m/z): [M+H]⁺=460.

### 5-Amino-2-((2-methoxyphenyl)amino)-8-phenylpyrido[2,3-d]pyrimidin-7(8H)-one

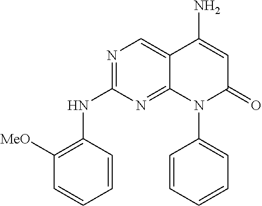 [SMILES: COc1ccccc1Nc1ncc2c(N)cc(=O)n(-c3ccccc3)c2n1]

A solution of tert-butyl (2-((2-methoxyphenyl)amino)-7-oxo-8-phenyl-7,8-dihydropyrido[2,3-d]pyrimidin-5-yl)carbamate (500 mg, 1.09 mmol, 1.0 eq.) and trifluoroacetic acid (5.0 mL) in dichloromethane (10 mL) was stirred at room temperature for 1 h. The reaction mixture was concentrated under reduced pressure to yield the title compound which was used without purification.

### N-(2-((2-Methoxyphenyl)amino)-7-oxo-8-phenyl-7,8-dihydropyrido[2,3-d]pyrimidin-5-yl)acrylamide

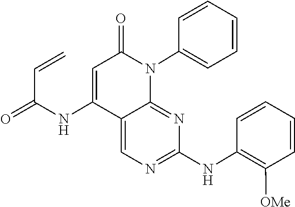 [SMILES: C=CC(=O)Nc1cc(=O)n(-c2ccccc2)c2nc(Nc3ccccc3OC)ncc12]

To a stirred solution of 5-amino-2-((2-methoxyphenyl)amino)-8-phenylpyrido[2,3-d]pyrimidin-7(8H)-one (90.0 mg, 0.250 mmol, 1.0 eq.), DMAP (3.00 mg, 25.0 μmol, 0.1 eq.), triethylamine (50.0 mg, 0.500 mmol, 2.0 eq.) and dichloromethane (5 mL) at 0° C. was added acryloyl chloride (23.0 mg, 0.250 mmol, 1.0 eq.) dropwise. The reaction was stirred at room temperature for 1 h before being diluted with water (25 mL) and dichloromethane (20 mL). The dichloromethane layer was separated, dried (Na₂SO₄) and concentrated under reduced pressure. The crude material was purified by reverse phase flash column chromatography eluting with acetonitrile (20-100%) in water (0.1% formic acid) to yield the title compound as a yellow solid (10.0 mg, 10%).

(ES, m/z): [M+H]⁺=414.

¹H NMR (400 MHz, CDCl₃) δ 10.74 (s, 1H), 9.21 (s, 1H), 8.24 (s, 4H), 7.59-7.54 (m, 3H), 7.37-7.31 (m, 3H), 6.96-6.89 (m, 2H), 6.43-6.45 (m, 1H), 3.81 (s, 3H), 2.82-2.79 (m, 2H), 2.68-2.60 (m, 2H).

### Example 3

### 6-(Methylsulfanyl)-4-(phenylamino)pyridine-3-carboxylic acid

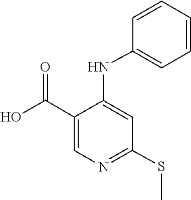 [SMILES: CSc1cc(Nc2ccccc2)c(C(=O)O)cn1]

A solution of ethyl 6-chloro-4-(phenylamino)pyridine-3-carboxylate (12.0 g, 43.4 mmol, 1.0 eq.) and sodium thiomethoxide (6.00 g) in DMF (50 mL) was stirred for 5 h at room temperature. The resulting mixture was diluted with water (50 mL). The resulting mixture was concentrated under reduced pressure to yield the title compound as an off-white solid (8.00 g, 71%).

### 5-(1,2,3-Benzotriazole-1-carbonyl)-2-(methylsulfanyl)-N-phenylpyridin-4-amine

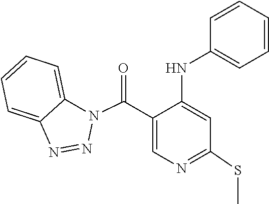 [SMILES: CSc1cc(Nc2ccccc2)c(C(=O)n2nnc3ccccc32)cn1]

General procedure 4 was applied to 6-(methylsulfanyl)-4-(phenylamino)pyridine-3-carboxylic acid (12.0 g, 46.1 mmol), benzotriazole (6.00 g, 50.4 mmol) and EDCl (13.0 g, 67.8 mmol) in dichloromethane (100 mL). The residue was purified by flash column chromatography, eluting with ethyl acetate (10%) in 40-60 petroleum ether to afford the title compound as a white solid (3.00 g, 18%).

### Ethyl 3-[6-(methylsulfanyl)-4-(phenylamino)pyridin-3-yl]-3-oxopropanoate

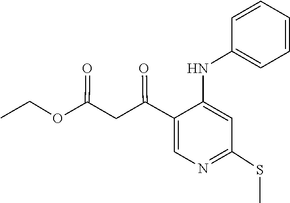 [SMILES: CCOC(=O)CC(=O)c1cnc(SC)cc1Nc1ccccc1]

General procedure 5 was applied to 5-(1,2,3-benzotriazole-1-carbonyl)-2-(methylsulfanyl)-N-phenylpyridin-4-amine (3.30 g, 9.13 mmol), ethyl acetate (2.30 mL, 23.5 mmol) and 1M LiHMDS in hexane (23.1.0 mL, 23.5 mmol) in THF (50 & 10 mL). The crude product was purified by flash column chromatography eluting with ethyl acetate (10%) in 40-60 petroleum ether to afford the title compound as an off-white solid (2.00 g, 66%).

### 4-Hydroxy-7-(methylsulfanyl)-1-phenyl-1,6-naphthyridin-2-one

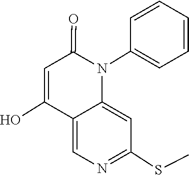 [SMILES: CSc1cc2c(cn1)c(O)cc(=O)n2-c1ccccc1]

General procedure 6 was applied to ethyl 3-[6-(methylsulfanyl)-4-(phenylamino)pyridin-3-yl]-3-oxopropanoate (2.00 g, 6.05 mmol) and DBU (0.50 mL, 3.35 mmol) in N,N-diisopropylethylamine (20 mL) to yield the title compound as an off-white solid (1.60 g, 93%).

### 7-(Methylsulfanyl)-2-oxo-1-phenyl-1,6-naphthyridin-4-yl trifluoromethanesulfonate

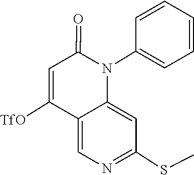 [SMILES: CSc1cc2c(cn1)c(OS(=O)(=O)C(F)(F)F)cc(=O)n2-c1ccccc1]

General procedure 8 was applied to 4-hydroxy-7-(methylsulfanyl)-1-phenyl-1,6-naphthyridin-2-one (1.60 g, 5.63 mmol), triethylamine (2.00 mL, 19.7 mmol) and triflic anhydride (2.00 g, 7.09 mmol) in dichloromethane. The precipitated solids were collected by filtration to yield the title compound as a brown solid (2.10 g, 90%).

### 7-(Methylsulfanyl)-1-phenyl-4-[2-(triisopropylsilyl)ethynyl]-1,6-naphthyridin-2-one

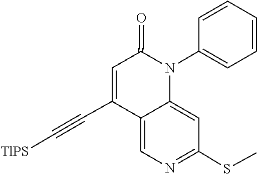

General procedure 9 was applied to triisopropylsilylacetylene (276 mg, 1.51 mmol) and 7-(methylsulfanyl)-2-oxo-1-phenyl-1,6-naphthyridin-4-yl trifluoromethanesulfonate (312 mg, 0.749 mmol), palladium(II) chloride (40.0 mg, 0.23 mmol), copper(I) iodide (40.0 mg, 0.21 mmol) and triphenylphosphine (90.0 mg, 0.205 mmol) in DMF (3.00 mL) and N,N-diisopropylethylamine (1.5 mL). The residue was purified by preparative TLC eluting with ethyl acetate (10%) in 40-60 petroleum ether to afford the title compound as a pink solid (200 mg, 59%).

### 7-[(2-Methoxyphenyl)amino]-1-phenyl-4-[2-(triisopropylsilyl)ethynyl]-1,6-naphthyridin-2-one

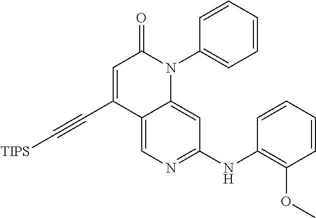

To a solution of 7-(methylsulfanyl)-1-phenyl-4-[2-(triisopropylsilyl)ethynyl]-1,6-naphthyridin-2-one (200 mg, 0.446 mmol, 1.0 eq.) and o-anisidine (110 mg, 0.893 mmol, 2.0 eq.) in toluene (5.0 mL) was added LiHMDS (1.20 mL, 7.17 mmol, 16 eq.) and nickeldicarbaldehyde (20.0 mg, 0.168 mmol, 0.4 eq.) and 1,2-bis(dicyclohexylphosphino)ethane (20.0 mg, 0.047 mmol, 0.1 eq.). After stirring for 1 day at 100° C. under a nitrogen atmosphere, the resulting mixture was concentrated under reduced pressure. The residue was purified by reverse phase flash column chromatography eluting with acetonitrile (10-50%) in water to yield the title compound as a yellow solid (50.0 mg, 21%). 4-Ethynyl-7-[(2-methoxyphenyl)amino]-1-phenyl-1,6-naphthyridin-2-one

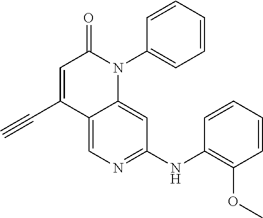 [SMILES: C#Cc1cc(=O)n(-c2ccccc2)c2cc(Nc3ccccc3OC)ncc12]

General procedure 15 was applied to 7-[(2-methoxyphenyl)amino]-1-phenyl-4-[2-(triisopropylsilyl)ethynyl]-1,6-naphthyridin-2-one (40.0 mg, 76.0 μmol) and potassium fluoride (24.0 mg, 0.411 mmol) in methanol (1.60 mL) and THF (1.60 mL). The resulting mixture was stirred for 4 h at room temperature and the crude residue was purified by reverse phase flash column chromatography eluting with methanol (10-50%) in water to yield the title compound as a yellow solid (15.0 mg, 53%).

(ES, m/z): [M+H]⁺=368.10.

¹H-NMR (300 MHz, DMSO-d₆) δ 8.69 (s, 1H), 8.63 (s, 1H), 7.73 (s, 1H), 7.71-7.53 (m, 3H), 7.33-7.31 (d, J=6.9 Hz, 2H), 7.00-6.85 (m, 3H), 6.58 (s, 1H), 5.89 (s, 1H), 5.04 (s, 1H), 3.72 (s, 3H).

### Example 4

### 2-Chloro-4-(phenylamino)pyrimidine-5-carboxamide

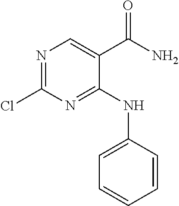 [SMILES: NC(=O)c1cnc(Cl)nc1Nc1ccccc1]

A solution of 2,4-dichloropyrimidine-5-carboxamide (10.0 g, 52.1 mmol, 1.0 eq.), triethylamine (7.90 g, 78.1 mmol, 1.5 eq.), aniline (5.82 g, 62.5 mmol, 1.2 eq.) in THF (200 mL) was stirred overnight at room temperature. The reaction was then quenched by the addition of water (150 mL) and extracted with ethyl acetate (200 mL). The resulting mixture was washed with water (3×50 mL), dried (Na₂SO₄) and concentrated under reduced pressure to yield the title compound as a white solid (12.0 g, 93%).

### 2-[(2-Methoxyphenyl)amino]-4-(phenylamino)pyrimidine-5-carboxamide

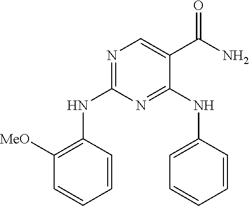 [SMILES: COc1ccccc1Nc1ncc(C(N)=O)c(Nc2ccccc2)n1]

A solution of 2-chloro-4-(phenylamino)pyrimidine-5-carboxamide (12.0 g, 48.4 mmol, 1.0 eq.), o-anisidine (7.15 g, 58.1 mmol, 1.2 eq.) and N,N-diisopropylethylamine (9.38 g, 72.6 mmol, 1.5 eq.) in acetonitrile (200 mL) was stirred for 3 h at 110° C. The reaction mixture was cooled to room temperature and concentrated under reduced pressure. The reaction was then quenched by the addition of water (500 mL) and the resulting solids were collected by filtration to yield the title compound as a yellow solid (15.0 g, 93%).

### 7-[(2-Methoxyphenyl)amino]-1-phenyl-3H-pyrimido[4,5-d][1,3]diazine-2,4-dione

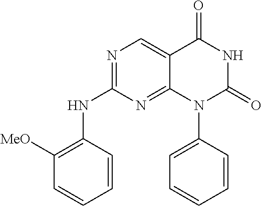 [SMILES: COc1ccccc1Nc1ncc2c(=O)[nH]c(=O)n(-c3ccccc3)c2n1]

A solution of 2-[(2-methoxyphenyl)amino]-4-(phenylamino)pyrimidine-5-carboxamide (10.0 g, 29.8 mmol, 1.0 eq.), CDI (14.5 g, 89.6 mmol, 3.0 eq.) and potassium carbonate (8.24 g, 59.7 mmol, 2.0 eq.) in THF was stirred for 2 h at room temperature before being concentrated under reduced pressure. The resulting material was diluted with water (500 mL) and the solids were collected by filtration to yield the title compound as an off-white solid (9.00 g, 46%).

### 4-Chloro-7-[(2-methoxyphenyl)amino]-1-phenylpyrimido[4,5-d][1,3]diazin-2-one

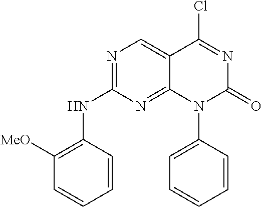 [SMILES: COc1ccccc1Nc1ncc2c(Cl)nc(=O)n(-c3ccccc3)c2n1]

A solution of 7-[(2-methoxyphenyl)amino]-1-phenyl-3H-pyrimido[4,5-d][1,3]diazine-2,4-dione (2.00 g, 5.53 mmol, 1.0 eq.) in acetonitirile, N,N-diisopropylethylamine (10 mL) and phosphorus(V) oxychloride (8.0 mL, 85.8 mmol, 16 eq.) was stirred for 30 min at 80° C. The resulting mixture was concentrated under reduced pressure. The pH of the solution was adjusted to 8 with aqueous ammonia and the solids were collected by filtration to yield the title compound as a yellow green solid (2.00 g, 95%).

### 7-[(2-Methoxyphenyl)amino]-1-phenyl-4-[2-(triisopropylsilyl)ethynyl]pyrimido[4,5-d][1,3]diazin-2-one

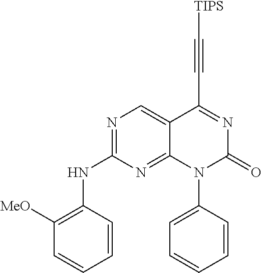

General procedure 9 was applied to 4-chloro-7-[(2-methoxyphenyl)amino]-1-phenylpyrimido[4,5-d][1,3]diazin-2-one (1.50 g, 3.95 mmol), triisopropylsilylacetylene (1.44 mg, 7.89 mmol), copper(I) iodide (75.5 mg, 0.395 mmol), bis(triphenylphosphine)palladium(II) dichloride (277 mg, 0.395 mmol), N,N-diisopropylethylamine (1.52 g, 11.8 mmol) in DMF. The crude product was purified by reverse phase HPLC eluting with acetonitrile (20-100%) in water (0.1% NH₄HCO₃) to yield the title compound as a yellow solid (1.20 g, 58%).

### 4-Ethynyl-7-[(2-methoxyphenyl)amino]-1-phenylpyrimido[4,5-d][1,3]diazin-2-one

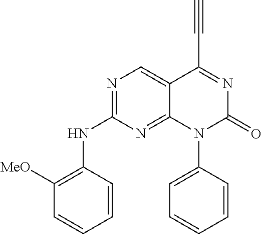 [SMILES: C#Cc1nc(=O)n(-c2ccccc2)c2nc(Nc3ccccc3OC)ncc12]

A solution of 7-[(2-methoxyphenyl)amino]-1-phenyl-4-[2-(triisopropylsilyl)ethynyl]pyrimido[4,5-d][1,3]diazin-2-one (100 mg, 0.190 mmol) and ammonium fluoride (141 mg, 3.80 mmol) in THF (2.0 mL) and water (20 uL) was stirred for 20 min at room temperature. The crude product was purified by reverse phase HPLC eluting with acetonitrile (20-100%) in water (0.05% formic acid) to yield the title compound as a brown solid (15.0 mg, 21%).

(ES, m/z): [M+H]⁺=370

¹H-NMR (300 Hz, CDCl₃) δ 9.01 (s, 1H), 8.40 (s, 1H), 7.84-7.56 (m, 4H), 7.46 (t, J=6.5 Hz, 2H), 6.98 (t, J=8.3 Hz, 1H), 6.85 (d, J=8.3 Hz, 1H), 6.52 (t, J=8.5 Hz, 2H), 3.90 (s, 3H), 3.80 (s, 1H).

### Example 5

### 2-((2-Methoxyphenyl)amino)-8-phenyl-5-((triisopropylsilyl)ethynyl)pyrido[2,3-d]pyrimidin-7(8H)-one

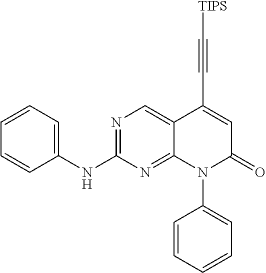

General procedure 13 was applied to 2-(methylsulfonyl)-8-phenyl-5-((triisopropylsilyl)ethynyl)pyrido[2,3-d]pyrimidin-7(8H)-one (250 mg, 0.52 mmol) with aniline (64 mg, 0.68 mmol), trifluoroacetic acid (60 mg, 0.52 mmol) and 2-butanol (2.0 mL). The crude material was purified by reverse phase flash column chromatography eluting with acetonitrile (10-100%) in water (0.1% NH₄HCO₃) to yield the title compound as a yellow solid (150 mg, 55%)

(ES, m/z): [M+H]⁺=495.3

### 5-Ethynyl-2-((2-methoxyphenyl)amino)-8-phenylpyrido[2,3-d]pyrimidin-7(8H)-one

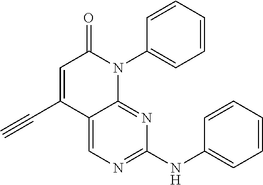 [SMILES: C#Cc1cc(=O)n(-c2ccccc2)c2nc(Nc3ccccc3)ncc12]

General procedure 15 was applied to 2-((2-methoxyphenyl)amino)-8-phenyl-5-((triisopropylsilyl)ethynyl)pyrido [2,3-d]pyrimidin-7(8H)-one (150 mg, 0.286 mmol), potassium fluoride (83.0 mg, 1.43 mmol), methanol (10.0 mL) and water (1.0 mL). The crude material was purified by reverse phase flash column chromatography eluting with acetonitrile (10-100%) in water (0.1% NH₄HCO₃) then lyophilized to yield the title compound as a yellow solid (30.4 mg, 28%).

(ES, m/z): [M+H]⁺=339.1

¹H-NMR (300 MHz, DMSO-d₆) δ 10.17 (s, 1H), 8.84 (s, 1H), 7.62-7.58 (t, J=6.0 Hz, 3H), 7.38-7.35 (dd, J=9.0 Hz, 2H), 7.29-7.26 (d, J=9.0 Hz, 2H), 6.99-6.94 (t, J=7.5 Hz, 2H), 6.87-6.85 (1H, d, J=6.0 Hz, 1H), 6.69 (s, 1H), 5.13 (s, 1H).

### Example 6

### 4-(4-Nitrophenyl)morpholine

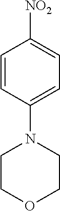 [SMILES: O=[N+]([O-])c1ccc(N2CCOCC2)cc1]

General procedure 11 was applied to 4-fluoro-1-nitrobenzene (1.55 g, 14.6 mmol) with morpholine (1.27 mL, 14.6 mmol) and potassium carbonate (3.30 g, 21.9 mmol) in DMSO (14.6 mL). The title compound, a bright yellow solid, was carried forward without further purification (2.29 g, 11.0 mmol, 75%).

(ES, m/z): [M+H]⁺=209.1

### 4-Morpholinoaniline

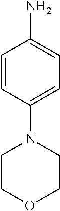 [SMILES: Nc1ccc(N2CCOCC2)cc1]

General procedure 12 was applied to 4-(4-nitrophenyl)morpholine (1.50 g, 7.20 mmol) with palladium on carbon 5 wt. % (380 mg, 0.360 mmol) in ethanol (36 mL). The crude material was purified by flash column chromatography eluting with methanol (0-20%) in dichloromethane to yield the title compound as a purple oil (1.28 g, 7.20 mmol, 100%).

### 2-((4-Morpholinophenyl)amino)-8-phenyl-5-((triisopropylsilyl)ethynyl)pyrido[2,3-d]pyrimidin-7(8H)-one

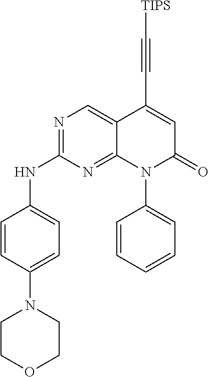

General procedure 13 was applied to 2-(methylsulfonyl)-8-phenyl-5-((triisopropylsilyl)ethynyl)pyrido[2,3-d]pyrimidin-7(8H)-one (500 mg, 1.04 mmol) with 4-morpholinoaniline (194 mg, 1.09 mmol) and trifluoroacetic acid (80.0 μL, 1.09 mmol) in acetonitrile (10 mL). The crude material was purified by flash column chromatography eluting with methanol (0-20%) in dichloromethane to yield the title compound as an orange solid (186 mg, 0.320 mmol, 31%).

(ES, m/z): [M+H]⁺=580.5

### 5-Ethynyl-2-((4-morpholinophenyl)amino)-8-phenylpyrido[2,3-d]pyrimidin-7(8H)-one

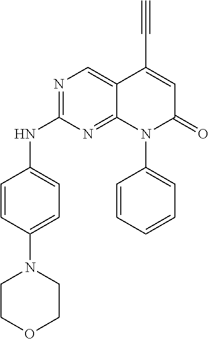 [SMILES: C#Cc1cc(=O)n(-c2ccccc2)c2nc(Nc3ccc(N4CCOCC4)cc3)ncc12]

General procedure 15 was applied to 2-((4-morpholinophenyl)amino)-8-phenyl-5-((triisopropylsilyl)ethynyl)pyrido[2,3-d]pyrimidin-7(8H)-one (113 mg, 0.190 mmol) with potassium fluoride (226 mg, 3.90 mmol) in DMF (2.0 mL) and methanol (2.0 mL). The crude material was purified by flash column chromatography eluting with methanol (0-20%) in dichloromethane to yield the title compound as a red solid (31.4 mg, 74.1 μmol, 39%).

(ES, m/z): [M+H]⁺=424.3

¹H NMR (500 MHz, DMSO-d₆) δ 10.07 (s, 1H), 8.79 (s, 1H), 7.60 (d, J=7.2 Hz, 3H), 7.41-7.29 (m, 2H), 7.12 (d, J=8.1 Hz, 2H), 6.64 (s, 1H), 6.53 (d, J=7.6 Hz, 2H), 5.13 (s, 1H), 3.72 (dd, J=5.8, 3.8 Hz, 4H), 2.96 (s, 4H).

### Example 7

### 3-Methoxy-N-methyl-4-nitroaniline

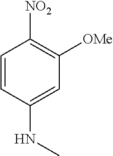

General procedure 11 was applied to 5-fluoro-2-nitroanisole (1.50 g, 8.77 mmol) with methyl amine hydrochloride (1.20 g, 17.5 mmol) and potassium carbonate (2.40 g, 17.5 mmol) in DMSO (6.0 mL). The title compound, a bright yellow solid, was carried forward without further purification (1.25 g, 6.89 mmol, 79%).

(ES, m/z): [M+H]=183.0

### 2-Chloro-N-(3-methoxy-4-nitrophenyl)-N-methylacetamide

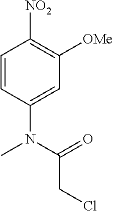 [SMILES: COc1cc(N(C)C(=O)CCl)ccc1[N+](=O)[O-]]

A solution of 3-methoxy-N-methyl-4-nitroaniline (1.42 g, 7.80 mmol, 1.0 eq.) and chloroacetyl chloride (740 μL, 9.36 mmol, 1.2 eq.) in ethyl acetate (16 mL, 0.5 M) was stirred at 70° C. for 1 h, then cooled to room temperature. Upon the addition of 40-60 petroleum ether, the precipitate formed was collected by vacuum filtration to yield the title compound (1.29 g, 4.99 mmol, 64%).

(ES, m/z): [M+H]⁺=259.1

### 2-(Dimethylamino)-N-(3-methoxy-4-nitrophenyl)-N-methylacetamide

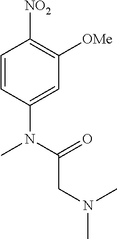 [SMILES: COc1cc(N(C)C(=O)CN(C)C)ccc1[N+](=O)[O-]]

A solution of 2-chloro-N-(3-methoxy-4-nitrophenyl)-N-methylacetamide (1.00 g, 3.87 mmol, 1.0 eq.), dimethylamine (13.5 mL, 13.5 mmol, 3.5 eq.) and potassium carbonate (1.87 g, 13.5 mmol, 3.5 eq.) in THF (77 mL, 0.05 M) and water (38 mL, 0.1 M) was stirred at room temperature overnight. The reaction mixture was quenched with saturated aqueous ammonium chloride solution (30 mL) and extracted with dichloromethane (3×30 mL). The combined organic layers were dried (MgSO₄) and concentrated under reduced pressure. The crude material was purified by flash column chromatography eluting with methanol (0-20%) in dichloromethane to yield the title compound (601 mg, 2.25 mmol, 58%).

(ES, m/z): [M+H]⁺=268.2

### N-(4-Amino-3-methoxyphenyl)-2-(dimethylamino)-N-methylacetamide

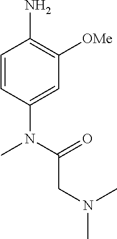 [SMILES: COc1cc(N(C)C(=O)CN(C)C)ccc1N]

General procedure 12 was applied to 2-(dimethylamino)-N-(3-methoxy-4-nitrophenyl)-N-methylacetamide (600 mg, 2.24 mmol) with palladium on carbon 5 wt. % (239 mg, 0.224 mmol) in ethanol (8.1.0 mL) and acetic acid (0.90 mL). The crude material was purified by flash column chromatography eluting with methanol (0-20%) in dichloromethane to yield the title compound as a purple oil (587 mg, 2.83 mmol, 100%).

### 2-(Dimethylamino)-N-(3-methoxy-4-((7-oxo-8-phenyl-5-((triisopropylsilyl)ethynyl)-7,8-dihydropyrido[2,3-d]pyrimidin-2-yl)amino)phenyl)-N-methylacetamide

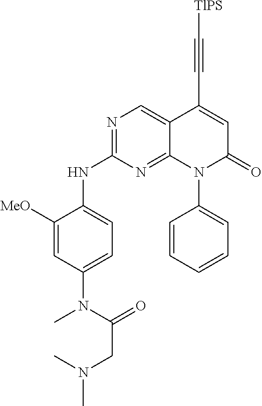

General procedure 13 was applied to 2-(methylsulfonyl)-8-phenyl-5-((triisopropylsilyl)ethynyl)pyrido[2,3-d]pyrimidin-7(8H)-one (500 mg, 1.04 mmol) with N-(4-amino-3-methoxyphenyl)-2-(dimethylamino)-N-methylacetamide (226 mg, 1.09 mmol) and trifluoroacetic acid (80.0 μL, 1.09 mmol) in acetonitrile (10 mL). The crude material was purified by flash column chromatography eluting with methanol (0-20%) in dichloromethane to yield the title compound as an orange solid (178 mg, 0.278 mmol, 27%).

(ES, m/z): [M+H]⁺=639.5

### 2-(Dimethylamino)-N-(4-((5-ethynyl-7-oxo-8-phenyl-7,8-dihydropyrido[2,3-d]pyrimidin-2-yl)amino)-3-methoxyphenyl)-N-methylacetamide

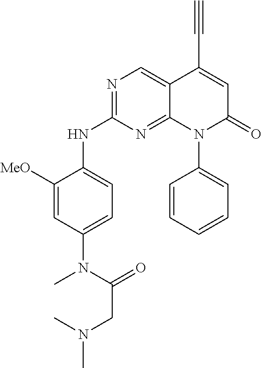 [SMILES: C#Cc1cc(=O)n(-c2ccccc2)c2nc(Nc3ccc(N(C)C(=O)CN(C)C)cc3OC)ncc12]

General procedure 15 was applied to 2-(dimethylamino)-N-(3-methoxy-4-((7-oxo-8-phenyl-5-((triisopropylsilyl)ethynyl)-7,8-dihydropyrido[2,3-d]pyrimidin-2-yl)amino)phenyl)-N-methylacetamide (50.0 mg, 78.3 μmol) with potassium fluoride (91 mg, 1.56 mmol) in DMF (1.0 mL). The crude material was purified by flash column chromatography eluting with methanol (0-20%) in dichloromethane to yield the title compound as a red solid (17.4 mg, 38.5 μmol, 49%).

(ES, m/z): [M+H]⁺=483.4

¹H NMR (500 MHz, CDCl₃) δ 8.85 (s, 1H), 7.98 (s, 1H), 7.60-7.45 (m, 3H), 7.42 (d, J=13.6 Hz, 1H), 7.28-7.22 (m, 2H), 6.72 (s, 1H), 6.57 (d, J=2.2 Hz, 1H), 6.19 (s, 1H), 3.81 (s, 3H), 3.65 (s, 1H), 3.14 (s, 3H), 2.83 (s, 2H), 2.26 (s, 6H).

### Example 8

### 2-Methoxy-N-(3-methoxy-4-nitrophenyl)-N-methylacetamide

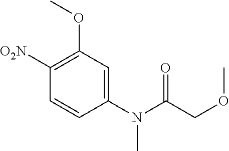 [SMILES: COCC(=O)N(C)c1ccc([N+](=O)[O-])c(OC)c1]

A solution of 3-methoxy-N-methyl-4-nitroaniline (0.41 g, 2.20 mmol, 1.0 eq.), 2-methoxyacetyl chloride (0.25 mL, 2.70 mmol, 1.2 eq.), N,N-diisopropylethylamine (0.77 mL, 4.40 mmol, 2.0 eq.) in dichloromethane (4.4 mL, 0.5 M) was stirred at 0° C. for 30 minutes then warmed to room temperature. After 1 h, additional 2-methoxyacetyl chloride (0.25 mL, 2.70 mmol, 1.2 eq.) was added and the reaction mixture was stirred for 1 h. The reaction mixture was quenched with cold water, extracted with dichloromethane, washed with brine, dried (MgSO₄), and concentrated under reduced pressure to yield the title compound as a yellow oil (0.59 g, 2.20 mmol, 100%).

(ES, m/z): [M+H]⁺=255.1

¹H NMR (500 MHz, DMSO-d₆) δ 7.95 (d, J=8.7 Hz, 1H), 7.40 (d, J=2.0 Hz, 1H), 7.12 (dd, J=8.7, 2.1 Hz, 1H), 3.94 (s, 3H), 3.23 (s, 6H).

### N-(4-Amino-3-methoxyphenyl)-2-methoxy-N-methylacetamide

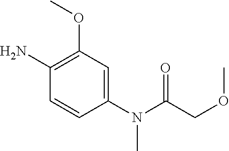 [SMILES: COCC(=O)N(C)c1ccc(N)c(OC)c1]

General procedure 12 was applied to 2-methoxy-N-(3-methoxy-4-nitrophenyl)-N-methylacetamide (0.59 g, 2.30 mmol) with palladium on carbon 5 wt. % (270 mg, 0.127 mmol) in methanol (9.3.0 mL). The crude material was purified by flash column chromatography eluting with methanol (0-10%) in dichloromethane to yield the title compound as a dark oil (270 mg, 1.19 mmol, 52%).

(ES, m/z): [M+H]⁺=225.2.

### 2-((2-Methoxyphenyl)amino)-8-phenyl-5-((triisopropylsilyl)ethynyl)pyrido [2,3-d]pyrimidin-7(8H)-one

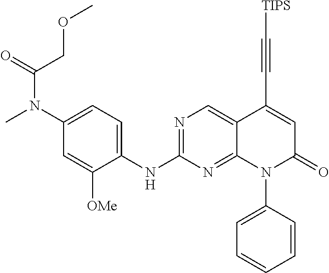

General procedure 13 was applied to 2-(methylsulfonyl)-8-phenyl-5-((triisopropylsilyl)ethynyl)pyrido[2,3-d]pyrimidin-7(8H)-one (350 mg, 0.72 mmol), N-(4-amino-3-methoxyphenyl)-2-methoxy-N-methylacetamide (163 mg, 0.72 mmol), trifluoroacetic acid (82.0 mg, 0.72 mmol) and 2-butanol (2.7 mL). The crude material was purified by flash column chromatography eluting with methanol (0-10%) in dichloromethane to yield the title compound as a red oil (200 mg, 0.32 mmol, 44%).

(ES, m/z): [M+H]⁺=626.5.

### N-(4-((5-Ethynyl-7-oxo-8-phenyl-7,8-dihydropyrido[2,3-d]pyrimidin-2-yl)amino)-3-methoxyphenyl)-2-methoxy-N-methylacetamide

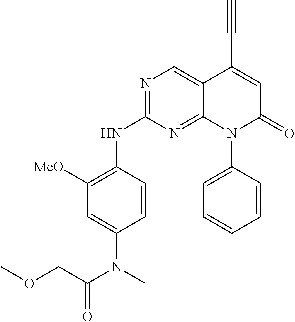 [SMILES: C#Cc1cc(=O)n(-c2ccccc2)c2nc(Nc3ccc(N(C)C(=O)COC)cc3OC)ncc12]

General procedure 15 was applied to 2-((2-methoxyphenyl)amino)-8-phenyl-5-((triisopropylsilyl)ethynyl)pyrido [2,3-d]pyrimidin-7(8H)-one (140 mg, 0.22 mmol) with potassium fluoride (65.0 mg, 1.10 mmol) in methanol (7.7 mL) and water (0.73.0 mL). The crude material was purified by flash column chromatography eluting with methanol (0-10%) in chloroform. The material was further purified by reverse phase HPLC eluting with acetonitrile (5-95%) in water to yield the title compound as a red solid (10.0 mg, 21.3 μmol, 10%)

(ES, m/z): [M+H]⁺=470.3.

¹H-NMR (500 MHz, MeOD-d₄) δ 8.99 (s, 1H), 7.66 (d, J=7.3 Hz, 3H), 7.58 (s, 1H), 7.45-7.30 (m, 2H), 6.93 (d, J=2.2 Hz, 1H), 6.75 (s, 1H), 6.39 (s, 2H), 4.66 (s, 1H), 3.93 (s, 3H), 3.79 (s, 2H), 3.23 (s, 3H)

### Example 9

### N¹,N¹,N³-Trimethyl-N³-(4-nitrophenyl)propane-1,3-diamine

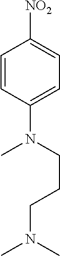

General procedure 11 was applied to 4-fluoro-1-nitrobenzene (1.00 g, 12.9 mmol) with N¹,N¹,N³-trimethylpropane-1,3-diamine (1.50 mL, 12.9 mmol) and potassium carbonate (3.25 g, 21.9 mmol) in DMSO (16 mL). The title compound, a bright yellow solid, was carried forward without further purification (1.00 g, 4.09 mmol, 32%).

### N¹-(3-(Dimethylamino)propyl)-N¹-methylbenzene-1,4-diamine

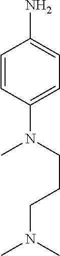

General procedure 12 was applied to N¹,N¹,N³-trimethyl-N³-(4-nitrophenyl)propane-1,3-diamine (1.00 g, 4.48 mmol) with palladium on carbon 5 wt. % (23.5 mg, 0.220 mmol) in ethanol (36 mL). The crude material was purified by flash column chromatography eluting with methanol (0-20%) in dichloromethane to yield the title compound as a purple oil (388 mg, 1.87 mmol, 42%).

### 2-((4-((3-(Dimethylamino)propyl)(methyl)amino)phenyl)amino)-8-phenyl-5-((triisopropylsilyl)ethynyl)pyrido[2,3-d]pyrimidin-7(8H)-one

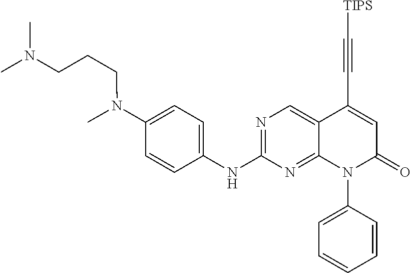

General procedure 13 was applied to 2-(methylsulfonyl)-8-phenyl-5-((triisopropylsilyl)ethynyl)pyrido[2,3-d]pyrimidin-7(8H)-one (600 mg, 1.25 mmol) with N¹-(3-(dimethylamino)propyl)-N¹-methylbenzene-1,4-diamine (388 mg, 1.87 mmol), trifluoroacetic acid (213 mg, 1.87 mmol) and acetonitrile (12.5 mL). The crude material was purified by flash column chromatography eluting with methanol (0-10%) in dichloromethane to yield the title compound as a red oil (260 mg, 0.431 mmol, 34%).

(ES, m/z): [M+H]⁺=609.5.

¹H-NMR (300 MHz, CDCl₃): δ 1.00 (s, 21H), 1.67 (qu, J=7.1 Hz, 2H), 2.24 (s, 6H), 2.35 (t, J=7.2 Hz, 2H), 2.74 (s, 3H), 3.18 (t, J=7.1 Hz, 2H), 6.20 (br s, 2H), 6.50 (s, 1H), 6.85 (br s, 2H), 7.14 (d, J=7.5 Hz, 2H), 7.50 (m, 3H), 8.70 (s, 3H)

### 2-((4-((3-(Dimethylamino)propyl)(methyl)amino)phenyl)amino)-5-ethynyl-8-phenylpyrido[2,3-d]pyrimidin-7(8H)-one

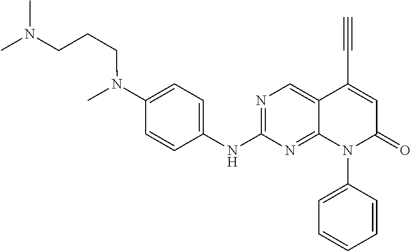 [SMILES: C#Cc1cc(=O)n(-c2ccccc2)c2nc(Nc3ccc(N(C)CCCN(C)C)cc3)ncc12]

General procedure 15 was applied to 2-((4-((3-(dimethylamino)propyl)(methyl)amino)phenyl)amino)-8-phenyl-5-((triisopropylsilyl)ethynyl)pyrido[2,3-d]pyrimidin-7(8H)-one (260 mg, 0.431 mmol) with potassium fluoride (495 mg, 8.59 mmol) in DMF (8.0 mL). The crude material was purified by flash column chromatography eluting with methanol (0-10%) in dichloromethane to yield the title compound as a red oil.

(ES, m/z): [M+H]⁺=453.3

¹H NMR (300 MHz, CDCl₃): δ 1.73 (q, J=7.2 Hz, 2H), 2.28 (s, 6H), 2.34 (t, J=7.3 Hz, 2H), 2.89 (s, 3H), 3.31 (t, J=7.2 Hz, 2H), 3.68 (s, 1H), 6.41 (br s, 2H), 6.72 (s, 1H), 7.02 (br s, 2H), 7.30 (m, 2H), 7.49-7.67 (m, 3H), 8.87 (s, 1H).

### Example 10

### 2-((1-(Methylsulfonyl)piperidin-4-yl)amino)-8-phenyl-5-((triisopropylsilyl)ethynyl)pyrido[2,3-d]pyrimidin-7(8H)-one

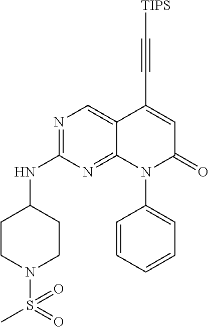

General procedure 14 was applied to 2-(methylsulfonyl)-8-phenyl-5-((triisopropylsilyl)ethynyl)pyrido[2,3-d]pyrimidin-7(8H)-one (200 mg, 0.415 mmol) with 4-amino-1-methane sulfonylpiperidine (111 mg, 0.623 mmol) and N,N-diisopropylethylamine (370 μL, 2.07 mmol) in DMSO (1.7 mL). The crude material was purified by flash column chromatography eluting with ethyl acetate (30-100%) in 40-60 petroleum ether to yield the title compound as a beige solid (110 mg, 0.190 mmol, 46%).

(ES, m/z): [M+H]=580.5.

### 5-Ethynyl-2-((1-(methylsulfonyl)piperidin-4-yl)amino)-8-phenylpyrido[2,3-d]pyrimidin-7(8H)-one

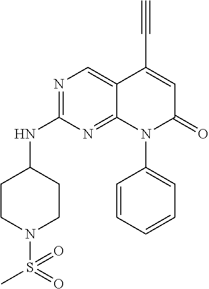 [SMILES: C#Cc1cc(=O)n(-c2ccccc2)c2nc(NC3CCN(S(C)(=O)=O)CC3)ncc12]

General procedure 15 was applied to 2-((1-(methylsulfonyl)piperidin-4-yl)amino)-8-phenyl-5-((triisopropylsilyl)ethynyl)pyrido[2,3-d]pyrimidin-7(8H)-one (100 mg, 0.172 mmol) with potassium fluoride (10.0 mg, 0.172 mmol) in methanol (1.7 mL) and dichloromethane (0.50 mL). The crude material was purified by flash column chromatography eluting with methanol (0-5%) in dichloromethane to yield the title compound as a beige solid (13.4 mg, 31.6 μmol, 18%).

(ES, m/z): [M+H]⁺=424.3.

¹H NMR (500 MHz, 100° C., DMSO-d₆) δ 8.72 (d, J=2.6 Hz, 1H), 7.51 (d, J=2.1 Hz, 2H), 7.46 (dd, J=8.5, 6.0 Hz, 1H), 7.27 (dd, J=7.8, 2.2 Hz, 2H), 6.52 (d, J=3.0 Hz, 1H), 4.81 (d, J=4.5 Hz, 1H), 3.50 (d, J=12.3 Hz, 2H), 2.83 (d, J=2.8 Hz, 3H), 2.62 (d, 2H), 1.77 (s, 2H), 1.47 (d, J=12.3 Hz, 3H).

### Example 11

### 2-((1′-Methyl-[1,4′-bipiperidin]-4-yl)amino)-8-phenyl-5-((triisopropylsilyl)ethynyl)pyrido[2,3-d]pyrimidin-7(8H)-one

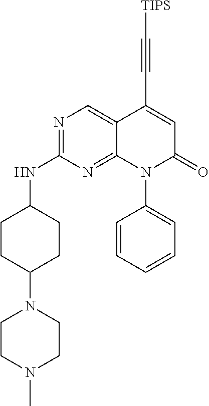

General procedure 14 was applied to 2-(methylsulfonyl)-8-phenyl-5-((triisopropylsilyl)ethynyl)pyrido[2,3-d]pyrimidin-7(8H)-one (500 mg, 1.04 mmol) with 1-methyl-1,4′-bipiperidin-4-amine (308 mg, 1.56 mmol) and N,N-diisopropylethylamine (930 μL, 5.20 mmol) in DMSO (4.2.0 mL). The crude material was purified by flash column chromatography eluting with methanol (0-20%) in dichloromethane to yield the title compound as an orange solid (284 mg, 0.474 mmol, 46%).

(ES, m/z): [M+H]⁺=599.7

### 5-Ethynyl-2-((1′-methyl-[1,4′-bipiperidin]-4-yl)amino)-8-phenylpyrido[2,3-d]pyrimidin-7(8H)-one

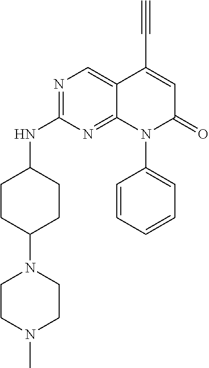 [SMILES: C#Cc1cc(=O)n(-c2ccccc2)c2nc(NC3CCC(N4CCN(C)CC4)CC3)ncc12]

General procedure 15 was applied to 2-((1′-methyl-[1,4′-bipiperidin]-4-yl)amino)-8-phenyl-5-((triisopropylsilyl)ethynyl)pyrido[2,3-d]pyrimidin-7(8H)-one (150 mg, 0.250 mmol) with potassium fluoride (291 mg, 5.00 mmol) in DMF (2.5 mL). The crude material was purified by flash column chromatography eluting with methanol (0-20%) in dichloromethane to yield the title compound as a red solid (32.0 mg, 72.4 μmol, 29%).

(ES, m/z): [M+H]⁺=443.5

¹H NMR (500 MHz, DMSO-d₆) δ 8.69 (d, J=34.4 Hz, 1H), 7.89 (d, J=6.8 Hz, 1H), 7.47 (dt, J=28.3, 7.8 Hz, 4H), 7.26 (d, J=7.5 Hz, 2H), 6.52 (d, J=7.2 Hz, 1H), 5.05 (s, 1H), 3.34 (s, 3H), 2.97-2.62 (m, 5H), 2.19 (d, J=5.5 Hz, 4H), 1.95 (s, 2H), 1.68 (dt, J=68.4, 14.9 Hz, 3H), 1.50-1.29 (m, 3H), 1.24 (d, J=9.1 Hz, 2H).

### Example 12

### 2-{[3-Methyl-4-(4-methylpiperazin-1-yl)phenyl]amino}-8-phenyl-5-[2-(triisopropylsilyl)ethynyl]pyrido[2,3-d]pyrimidin-7-one

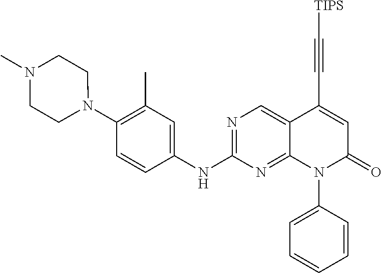

General procedure 13 was applied to 2-methanesulfonyl-8-phenyl-5-[2-(triisopropylsilyl)ethynyl]pyrido[2,3-d]pyrimidin-7-one (600 mg, 1.25 mmol) with trifluoroacetic acid (284 mg, 2.49 mmol) and 3-methyl-4-(4-methylpiperazin-1-yl)aniline (307 mg, 1.49 mmol) in 2-butanol (6.0 mL). The resulting solution was diluted with methanol (10 mL) and applied onto a reverse phase column directly eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (400 mg, 53%).

(ES, m/z): [M+H]⁺=607.4.

### 5-Ethynyl-2-{[3-methyl-4-(4-methylpiperazin-1-yl)phenyl]amino}-8-phenylpyrido[2,3-d]pyrimidin-7-one

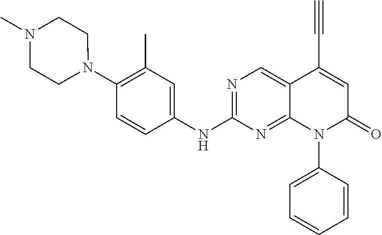 [SMILES: C#Cc1cc(=O)n(-c2ccccc2)c2nc(Nc3ccc(N4CCN(C)CC4)c(C)c3)ncc12]

General procedure 15 was applied to 2-{[3-methyl-4-(4-methylpiperazin-1-yl)phenyl]amino}-8-phenyl-5-[2-(triisopropylsilyl)ethynyl]pyrido[2,3-d]pyrimidin-7-one (400 mg, 0.659 mmol) and potassium fluoride (431 mg, 7.41 mmol) in THF (5.0 mL), methanol (2.5 mL) and water (0.50 mL). The reaction mixture was applied onto a reverse phase column directly, eluting with acetonitrile (20-100%) in water (0.1% formic acid). The desired fractions were collected and concentrated under reduced pressure to remove most of the acetonitrile then lyophilized to yield the title compound as an orange solid (137 mg, 46%).

(ES, m/z): [M+H]⁺=451.2

¹H-NMR (300 MHz, DMSO-d₆) δ 10.01 (s, 1H), 8.81 (s, 1H), 7.58-7.39 (m, 5H), 7.15-7.03 (m, 2H), 6.65 (s, 2H), 5.11 (s, 1H), 2.72 (d, J=4.9 Hz, 4H), 2.27 (s, 3H), 1.98 (s, 3H).

### Example 13

### 2-({4-[2-(dimethylamino)ethoxy]phenyl}amino)-8-phenyl-5-[2-(triisopropylsilyl)ethynyl]pyrido[2,3-d]pyrimidin-7-one

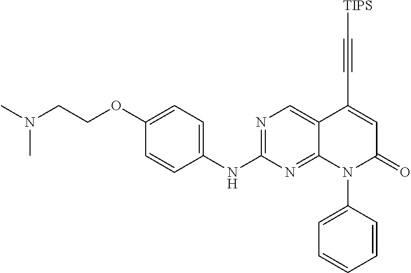

General procedure 13 was applied to 2-methanesulfonyl-8-phenyl-5-[2-(triisopropylsilyl)ethynyl]pyrido[2,3-d]pyrimidin-7-one (600 mg, 1.25 mmol) with trifluoroacetic acid (284 mg, 2.49 mmol) and 4-[2-(dimethylamino)ethoxy]aniline (269 mg, 1.49 mmol) in 2-butanol (6.0 mL). The resulting solution was stirred for 3 h at 100° C. then diluted with methanol (10 mL) and applied onto a reverse phase column directly, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (400 mg, 55%).

(ES, m/z): [M+H]⁺=582.3.

### 2-({4-[2-(Dimethylamino)ethoxy]phenyl}amino)-5-ethynyl-8-phenylpyrido[2,3-d]pyrimidin-7-one

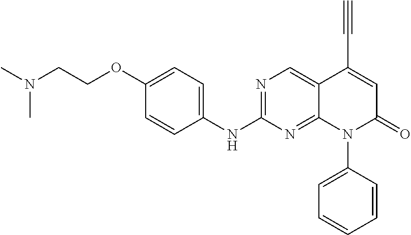 [SMILES: C#Cc1cc(=O)n(-c2ccccc2)c2nc(Nc3ccc(OCCN(C)C)cc3)ncc12]

General procedure 15 was applied to 2-({4-[2-(dimethylamino)ethoxy]phenyl}amino)-8-phenyl-5-[2-(triisopropylsilyl)ethynyl]pyrido[2,3-d]pyrimidin-7-one (400 mg, 0.687 mmol), potassium fluoride (449 mg, 7.73 mmol) in THF (5.0 mL), methanol (2.5 mL) and water (0.50 mL). The resulting solution was stirred for 2 h at 70° C. and purified by reverse phase flash column chromatography, eluting with acetonitrile (20-100%) in water (0.1% formic acid). The collected fractions were concentrated under reduced pressure to remove most of the acetonitrile and lyophilized to yield the title compound as a yellow solid (147 mg, 50%).

(ES, m/z): [M+H]=426.1

¹H-NMR (300 MHz, DMSO-d₆) δ 10.08 (s, 1H), 8.80 (s, 1H), 7.59 (d, J=6.7 Hz, 3H), 7.39-7.26 (m, 2H), 7.18 (s, 2H), 6.65 (s, 1H), 6.54 (s, 2H), 5.11 (s, 1H), 3.97 (t, J=5.7 Hz, 2H), 3.81 (s, 1H), 2.70 (t, J=5.7 Hz, 2H), 2.30 (s, 6H).

### Example 14

### tert-Butyl 4-[4-({7-oxo-8-phenyl-5-[2-(triisopropylsilyl)ethynyl]pyrido[2,3-d]pyrimidin-2-yl}amino)phenyl]piperazine-1-carboxylate

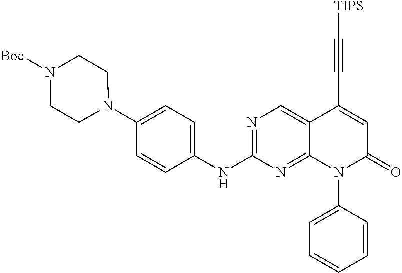

General procedure 13 was applied to 2-methanesulfonyl-8-phenyl-5-[2-(triisopropylsilyl)ethynyl]pyrido[2,3-d]pyrimidin-7-one (500 mg, 1.04 mmol) with trifluoroacetic acid (237 mg, 2.08 mmol) and tert-butyl 4-(4-aminophenyl)piperazine-1-carboxylate (345 mg, 1.25 mmol) in 2-butanol (5.0 mL). The resulting solution was stirred for 3 h at 100° C. The resulting solution was diluted with methanol (10 mL) and purified by reverse phase flash column chromatography, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (300 mg, 43%).

(ES, m/z): [M+H]⁺=679.4

### tert-Butyl 4-[4-({5-ethynyl-7-oxo-8-phenylpyrido[2,3-d]pyrimidin-2-yl}amino)phenyl]piperazine-1-carboxylate

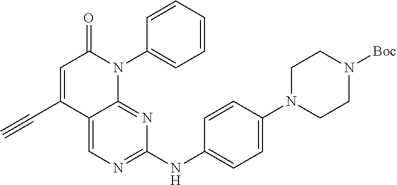 [SMILES: C#Cc1cc(=O)n(-c2ccccc2)c2nc(Nc3ccc(N4CCN(C(=O)OC(C)(C)C)CC4)cc3)ncc12]

General procedure 15 was applied to tert-butyl 4-[4-({7-oxo-8-phenyl-5-[2-(triisopropylsilyl)ethynyl]pyrido[2,3-d]pyrimidin-2-yl}amino)phenyl]piperazine-1-carboxylate (300 mg, 0.442 mmol) and potassium fluoride (256 mg, 4.42 mmol) in THF (3.0 mL), methanol (3.0 mL) and water (0.50 mL). The resulting solution was stirred for 2 h at 70° C. before being concentrated under reduced pressure to yield the title compound as a yellow solid (180 mg, 78%).

(ES, m/z): [M+H]⁺=523.2

### 5-Ethynyl-8-phenyl-2-{[4-(piperazin-1-yl)phenyl]amino}pyrido[2,3-d]pyrimidin-7-one

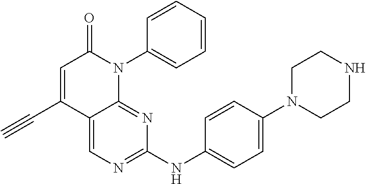 [SMILES: C#Cc1cc(=O)n(-c2ccccc2)c2nc(Nc3ccc(N4CCNCC4)cc3)ncc12]

A solution of tert-butyl 4-[4-({5-ethynyl-7-oxo-8-phenylpyrido[2,3-d]pyrimidin-2-yl}amino)phenyl]piperazine-1-carboxylate (180 mg, 0.344 mmol, 1.0 eq.), hydrochloric acid (in methanol) (4.0 mL) was stirred for 8 h at 30° C. The reaction mixture was applied onto a reverse phase column directly, eluting with acetonitrile (20-100%) in water (0.1% formic acid). The desired fractions were collected and concentrated under reduced pressure to remove most of the acetonitrile then lyophilized to dryness to yield the title compound as an orange solid (79.4 mg, 54%).

(ES, m/z): [M+H]⁺=423.2

¹H-NMR (300 MHz, DMSO-d₆) δ 10.04 (s, 1H), 9.16-8.69 (m, 1H), 7.58 (q, J=8.7, 6.6 Hz, 4H), 7.42-7.25 (m, 2H), 7.14 (s, 2H), 6.67-6.34 (m, 3H), 5.10 (s, 1H), 3.53 (s, 1H), 3.00 (q, J=6.1 Hz, 8H).

### Example 15

### 2-{[4-(4-Ethylpiperazin-1-yl)phenyl]amino}-8-phenyl-5-[2-(triisopropylsilyl)ethynyl]pyrido[2,3-d]pyrimidin-7-one

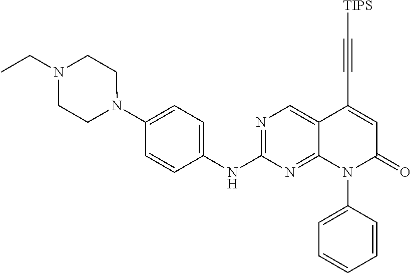

General procedure 13 was applied to 2-methanesulfonyl-8-phenyl-5-[2-(triisopropylsilyl)ethynyl]pyrido[2,3-d]pyrimidin-7-one (400 mg, 0.830 mmol) with trifluoroacetic acid (189 mg, 1.66 mmol) and 4-(4-ethylpiperazin-1-yl)aniline (205 mg, 0.996 mmol) in 2-butanol (4.0 mL). The resulting solution was stirred for 3 h at 100° C. then diluted with methanol (10 mL) and applied onto a reverse phase column directly, eluting with acetonitrile (70-100%) in water (0.1% trifluoroacetic acid). The resulting solution was basified to pH 8 with saturated aqueous sodium hydrogen carbonate solution then extracted with ethyl acetate (3×200 mL). The combined organic layers were washed with brine (2×200 mL), dried (Na₂SO₄) and concentrated under reduced pressure to yield the title compound as an orange solid (240 mg, 48%).

(ES, m/z): [M+H]⁺=607.4

### 2-{[4-(4-Ethylpiperazin-1-yl)phenyl]amino}-5-ethynyl-8-phenylpyrido[2,3-d]pyrimidin-7-one

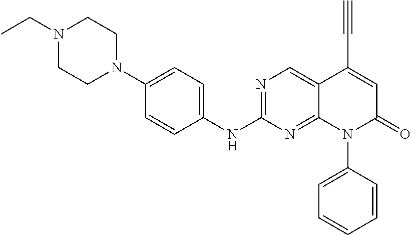 [SMILES: C#Cc1cc(=O)n(-c2ccccc2)c2nc(Nc3ccc(N4CCN(CC)CC4)cc3)ncc12]

General procedure 15 was applied to 2-{[4-(4-ethylpiperazin-1-yl)phenyl]amino}-8-phenyl-5-[2-(triisopropylsilyl)ethynyl]pyrido[2,3-d]pyrimidin-7-one (240 mg, 0.395 mmol) with potassium fluoride (230 mg, 3.95 mmol) in THF (2.0 mL), methanol (1.0 mL) and water (0.50 mL). The resulting solution was stirred for 2 h at 70° C. The reaction mixture was applied onto a reverse phase column directly, eluting with acetonitrile (20-100%) in water (0.1% formic acid). The desired fractions were collected and concentrated under reduced pressure to remove most of the acetonitrile then lyophilized to yield the title compound as an orange solid (42.3 mg, 24%).

(ES, m/z): [M+H]⁺=451.1

¹H-NMR (300 MHz, DMSO-d₆) δ 10.01 (s, 1H), 8.78 (s, 1H), 7.59 (d, J=6.7 Hz, 3H), 7.40-7.29 (m, 2H), 7.11 (d, J=8.3 Hz, 2H), 6.58 (d, J=27.2 Hz, 3H), 5.10 (s, 1H), 3.00 (t, J=4.8 Hz, 4H), 2.53 (d, J=3.7 Hz, 4H), 2.40 (q, J=7.2 Hz, 2H), 1.04 (t, J=7.1 Hz, 3H).

### Example 16

### 2-{[4-(4-Isopropylpiperazin-1-yl)phenyl]amino}-8-phenyl-5-[2-(triisopropylsilyl)ethynyl]pyrido[2,3-d]pyrimidin-7-one

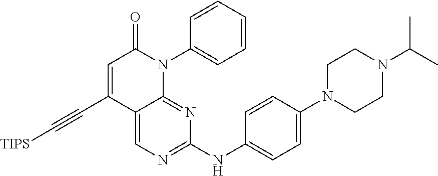

General procedure 13 was applied to 2-methanesulfonyl-8-phenyl-5-[2-(triisopropylsilyl)ethynyl]pyrido[2,3-d]pyrimidin-7-one (300 mg, 0.617 mmol) with 4-(4-isopropylpiperazin-1-yl)aniline (203 mg, 0.925 mmol) and trifluoroacetic acid (176 mg, 1.54 mmol) in 2-butanol (3.0 mL). The reaction mixture was stirred at 110° C. for 24 h then applied onto a reverse phase column directly, eluting with eluting with acetonitrile (10-100%) in water (0.1% trifluoroacetic acid) to yield the title compound as a yellow solid (250 mg, 65%).

(ES, m/z): [M+H]⁺=621.3

### 5-Ethynyl-2-{[4-(4-isopropylpiperazin-1-yl)phenyl]amino}-8-phenylpyrido[2,3-d]pyrimidin-7-one

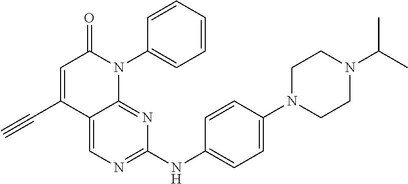 [SMILES: C#Cc1cc(=O)n(-c2ccccc2)c2nc(Nc3ccc(N4CCN(C(C)C)CC4)cc3)ncc12]

General procedure 15 was applied to 2-{[4-(4-isopropylpiperazin-1-yl)phenyl]amino}-8-phenyl-5-[2-(triisopropylsilyl) ethynyl]pyrido[2,3-d]pyrimidin-7-one (250 mg, 0.401 mmol) with potassium fluoride (233 mg, 4.01 mmol) in THF (2.5 mL), methanol (1.25 mL) and water (0.20 mL). The reaction mixture was applied onto a reverse phase column directly, eluting with acetonitrile (10-100%) in water (0.1% formic acid). The desired fractions were collected and concentrated under reduced pressure to remove most of the acetonitrile then lyophilized to dryness to yield the title compound as a yellow solid (65.9 mg, 35%).

(ES, m/z): [M+H]⁺=465.2

¹H NMR (300 MHz, DMSO-d₆) δ 10.02 (s, 1H), 8.79 (s, 1H), 8.20 (s, 1H), 7.59 (d, J=6.7 Hz, 3H), 7.40-7.27 (m, 2H), 7.12 (s, 2H), 6.63 (s, 1H), 6.53 (s, 2H), 5.10 (s, 1H), 3.00 (t, J=4.8 Hz, 4H), 2.72 (quin, J=6.5 Hz, 1H), 2.61 (t, J=5.0 Hz, 4H), 1.03 (d, J=6.5 Hz, 6H).

### Example 17

### 2-({4-[4-(2-Hydroxyethyl)piperazin-1-yl]phenyl}amino)-8-phenyl-5-[2-(triisopropylsilyl)ethynyl]pyrido[2,3-d]pyrimidin-7-one

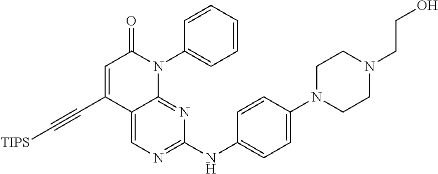

General procedure 13 was applied to 2-methanesulfonyl-8-phenyl-5-[2-(triisopropylsilyl)ethynyl]pyrido[2,3-d]pyrimidin-7-one (450 mg, 0.897 mmol) with 2-[4-(4-aminophenyl)piperazin-1-yl]ethanol (238 mg, 1.07 mmol) and trifluoroacetic acid (205 mg, 1.79 mmol) in 2-butanol (4.5 mL). The solution mixture was stirred for 2 h at 100° C. The residue was purified by reverse phase flash column chromatography eluting with acetonitrile (30-60%) in water (0.1% formic acid) to yield the title compound as an orange solid (310 mg, 54%).

(ES, m/z): [M+H]⁺=623.3.

### 5-ethynyl-2-({4-[4-(2-hydroxyethyl) piperazin-1-yl]phenyl}amino)-8-phenylpyrido[2,3-d]pyrimidin-7-one

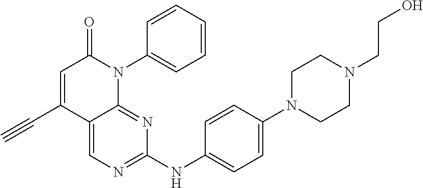 [SMILES: C#Cc1cc(=O)n(-c2ccccc2)c2nc(Nc3ccc(N4CCN(CCO)CC4)cc3)ncc12]

General procedure 15 was applied to 2-({4-[4-(2-hydroxyethyl)piperazin-1-yl]phenyl}amino)-8-phenyl-5-[2-(triisopropylsilyl)ethynyl]pyrido[2,3-d]pyrimidin-7-one (300 mg, 0.472 mmol) with potassium fluoride (548 mg, 9.44 mmol) in THF (3.0 mL) and methanol (1.5 mL). The resulting mixture was stirred for 2 h at 80° C. The residue was purified by reverse phase flash column chromatography eluting with acetonitrile (30-60%) in water (0.1% formic acid) to yield the title compound as a brown solid (41.0 mg, 18%).

(ES, m/z): [M+H]⁺=467.2.

¹H NMR (400 MHz, DMSO-d₆) δ 10.03 (s, 1H), 8.78 (s, 1H), 7.59 (d, J=7.0 Hz, 3H), 7.34 (d, J=7.1 Hz, 2H), 7.11 (d, J=8.2 Hz, 2H), 6.63 (s, 1H), 6.52 (s, 2H), 5.10 (s, 1H), 3.54 (d, J=12.2 Hz, 2H), 2.98 (d, J=5.8 Hz, 4H), 2.56 (t, J=4.7 Hz, 4H), 2.44 (d, J=6.2 Hz, 2H).

### Example 18

### 2-({4-[4-(Oxetan-3-yl)piperazin-1-yl]phenyl}amino)-8-phenyl-5-[2-(triisopropylsilyl)ethynyl]pyrido[2,3-d]pyrimidin-7-one

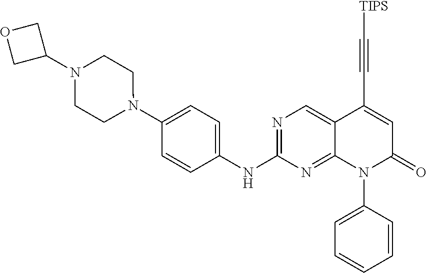

General procedure 13 was applied to 2-methanesulfonyl-8-phenyl-5-[2-(triisopropylsilyl)ethynyl]pyrido[2,3-d]pyrimidin-7-one (400 mg, 0.830 mmol) with trifluoroacetic acid (189 mg, 1.66 mmol) and 4-[4-(oxetan-3-yl)piperazin-1-yl]aniline (233 mg, 0.996 mmol) in 2-butanol (4.0 mL). The resulting solution was stirred for 3 h at 100° C. then diluted with methanol (10 mL) and applied onto a reverse phase column directly, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (130 mg, 25%).

(ES, m/z): [M+H]⁺=635.3

### 5-Ethynyl-2-({4-[4-(oxetan-3-yl)piperazin-1-yl]phenyl}amino)-8-phenylpyrido[2,3-d]pyrimidin-7-one

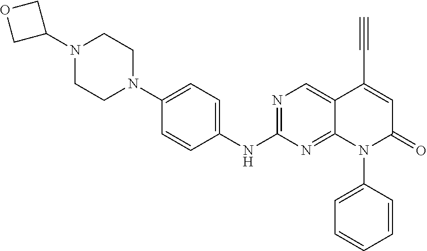 [SMILES: C#Cc1cc(=O)n(-c2ccccc2)c2nc(Nc3ccc(N4CCN(C5COC5)CC4)cc3)ncc12]

General procedure 15 was applied to 2-({4-[4-(oxetan-3-yl)piperazin-1-yl]phenyl}amino)-8-phenyl-5-[2-(triisopropylsilyl)ethynyl]pyrido[2,3-d]pyrimidin-7-one (130 mg, 0.205 mmol) with potassium fluoride (119 mg, 2.05 mmol) in THF (2.0 mL), methanol (1.0 mL) and water (0.50 mL). The resulting solution was stirred for 2 h at 70° C. then applied onto a reverse phase column directly, eluting with acetonitrile (20-100%) in water (0.1% formic acid). The desired fractions were collected and concentrated under reduced pressure to remove most of the acetonitrile then lyophilized to yield the title compound as an orange solid (45.5 mg, 46%).

(ES, m/z): [M+H]⁺=479.1

¹H-NMR (300 MHz, DMSO-d₆): δ 10.02 (s, 1H), 8.78 (s, 1H), 7.59 (d, J=7.2 Hz, 3H), 7.38-7.29 (m, 2H), 7.11 (s, 1H), 6.63 (s, 1H), 6.54 (s, 2H), 5.10 (s, 1H), 4.57 (t, J=6.5 Hz, 2H), 4.46 (t, J=6.0 Hz, 11H).

### Example 19

### 2-{[1-(1-Methylpiperidin-4-yl)pyrazol-4-yl]amino}-8-phenyl-5-[2-(triisopropylsilyl)ethynyl]pyrido[2,3-d]pyrimidin-7-one

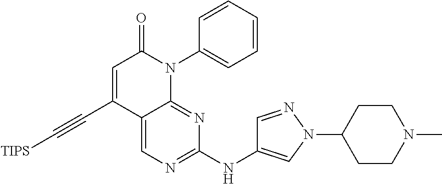

General procedure 13 was applied to 2-methanesulfonyl-8-phenyl-5-[2-(triisopropylsilyl)ethynyl]pyrido[2,3-d]pyrimidin-7-one (300 mg, 0.623 mmol) with 1-(1-methylpiperidin-4-yl)pyrazol-4-amine (135 mg, 0.748 mmol) and trifluoroacetic acid (142 mg, 1.25 mmol) in 2-butanol (3.0 mL). The solution mixture was stirred for 1 h at 100° C. The residue was purified by reverse phase flash column chromatography eluting with acetonitrile (30-60%) in water (0.1% formic acid) to yield the title compound as a yellow solid (210 mg, 58%).

(ES, m/z): [M+H]⁺=528.3

### 5-Ethynyl-2-{[1-(1-methylpiperidin-4-yl)pyrazol-4-yl]amino}-8-phenylpyrido[2,3-d]pyrimidin-7-one

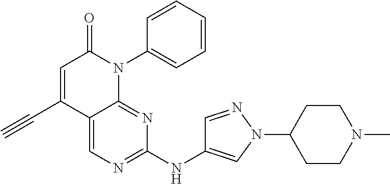 [SMILES: C#Cc1cc(=O)n(-c2ccccc2)c2nc(Nc3cnn(C4CCN(C)CC4)c3)ncc12]

General procedure 15 was applied to 2-{[1-(1-methylpiperidin-4-yl)pyrazol-4-yl]amino}-8-phenyl-5-[2-(triisopropylsilyl)ethynyl]pyrido[2,3-d]pyrimidin-7-one (210 mg, 0.361 mmol) with potassium fluoride (419 mg, 7.22 mmol) in THF (2.0 mL) and methanol (1.0 mL). The resulting mixture was stirred for 2 h at 80° C. The residue was purified by reverse phase flash column chromatography eluting with acetonitrile (30-60%) in water to yield the title compound as a yellow solid (59.0 mg, 38%).

(ES m/z): [M+H]⁺=426.1

¹H NMR (400 MHz, DMSO-d₆) δ 10.23 (s, 1H), 8.78 (s, 1H), 7.63 (dt, J=12.4, 6.8 Hz, 3H), 7.41-7.37 (m, 2H), 7.14 (s, 1H), 6.75 (s, 1H), 6.62 (s, 1H), 5.10 (s, 1H), 3.64-3.56 (m, 1H), 2.88 (d, J=11.4 Hz, 2H), 2.25 (s, 3H), 2.03 (td, J=11.7, 2.7 Hz, 2H), 1.78-1.63 (m, 4H).

### Example 20

### tert-Butyl 4-(4-nitrophenyl)-1,4-diazepane-1-carboxylate

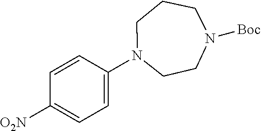 [SMILES: CC(C)(C)OC(=O)N1CCCN(c2ccc([N+](=O)[O-])cc2)CC1]

General procedure 11 was applied to 4-fluoronitrobenzene (2.00 g, 14.2 mmol) with tert-butyl 1,4-diazepane-1-carboxylate (3.41 g, 17.0 mmol) and potassium carbonate (3.92 g, 28.3 mmol) in DMF (20 mL). The crude material was slurried with 40-60 petroleum ether:ethyl acetate (1:1, 50 mL) and filtered, washing with 40-60 petroleum ether and dried under vacuum to yield the title compound as a yellow solid (3.50 g, 77%).

(ES, m/z): [M+H]⁺=322.2.

### tert-Butyl 4-(4-aminophenyl)-1,4-diazepane-1-carboxylate

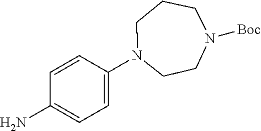 [SMILES: CC(C)(C)OC(=O)N1CCCN(c2ccc(N)cc2)CC1]

General procedure 12 was applied to tert-butyl 4-(4-nitrophenyl)-1,4-diazepane-1-carboxylate (1.00 g, 3.11 mmol) with palladium on carbon 5 wt. % (100 mg, 0.940 mmol) in methanol (20 mL) to yield the title compound as a black oil (800 mg, 88%).

(ES, m/z): [M+H]⁺=292.2.

### tert-Butyl 4-[4-({7-oxo-8-phenyl-5-[2-(triisopropylsilyl)ethynyl]pyrido[2,3-d]pyrimidin-2-yl}amino)phenyl]-1,4-diazepane-1-carboxylate

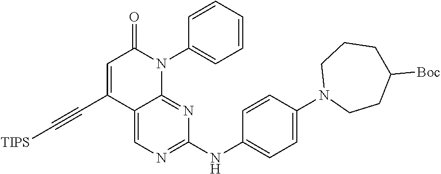

General procedure 13 was applied to 2-methanesulfonyl-8-phenyl-5-[2-(triisopropylsilyl)ethynyl]pyrido[2,3-d]pyrimidin-7-one (500 mg, 1.04 mmol) with tert-butyl 4-(4-aminophenyl)-1,4-diazepane-1-carboxylate (363 mg, 1.25 mmol) and trifluoroacetic acid (237 mg, 2.08 mmol) in 2-butanol (5.0 mL). The resulting solution was stirred for 3 h at 100° C. then diluted with methanol (10 mL) and applied onto a reverse phase column directly, eluting with acetonitrile (70-100%) in water (0.1% trifluoroacetic acid). The resulting solution was basified to pH 8 with saturated aqueous sodium hydrogen carbonate solution then extracted with ethyl acetate (3×200 mL). The combined organic layers were washed with brine (2×200 mL), dried (Na₂SO₄) and concentrated under reduced pressure to yield the title compound as a red solid (300 mg, 42%).

(ES, m/z): [M+H]⁺=693.4.

### 2-{[4-(1,4-Diazepan-1-yl)phenyl]amino}-8-phenyl-5-[2-(triisopropylsilyl)ethynyl]pyrido[2,3-d]pyrimidin-7-one

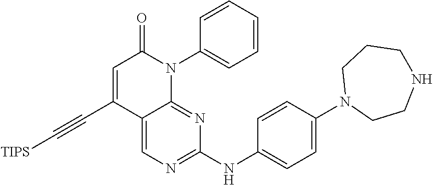

A solution of tert-butyl 4-[4-({7-oxo-8-phenyl-5-[2-(triisopropylsilyl)ethynyl]pyrido[2,3-d]pyrimidin-2-yl}amino)phenyl]-1,4-diazepane-1-carboxylate (300 mg, 0.433 mmol, 1.0 eq.) in hydrochloric acid in methanol (2.0 mL) was stirred for 5 h at 40° C. The resulting reaction mixture was concentrated under reduced pressure to yield the title compound as a white solid (256 mg, 100%).

(ES, m/z): [M+H]⁺=593.3.

### 2-{[4-(1,4-Diazepan-1-yl)phenyl]amino}-5-ethynyl-8-phenylpyrido[2,3-d]pyrimidin-7-one

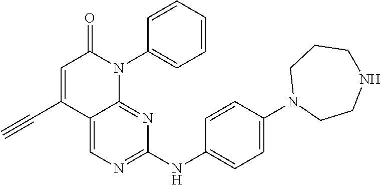 [SMILES: C#Cc1cc(=O)n(-c2ccccc2)c2nc(Nc3ccc(N4CCCNCC4)cc3)ncc12]

General procedure 15 was applied to 2-{[4-(1,4-diazepan-1-yl)phenyl]amino}-8-phenyl-5-[2-(triisopropylsilyl)ethynyl]pyrido[2,3-d]pyrimidin-7-one (200 mg, 0.337 mmol) with potassium fluoride (196 mg, 3.37 mmol) in methanol (1.0 mL) and THF (1.0 mL). The resulting solution was stirred for 2 h at 50° C., applied directly onto a reverse phase column, eluting with acetonitrile (20-100%) in water (0.1% formic acid). The desired fractions were collected and concentrated under reduced pressure to remove most of the acetonitrile then lyophilized to yield the title compound as an orange solid (120 mg, 81%).

(ES, m/z): [M+H]⁺=437.0.

¹H-NMR (400 MHz, DMSO-d₆) δ 9.93 (s, 1H), 8.75 (s, 1H), 7.60 (s, 3H), 7.34 (s, 2H), 7.07 (s, 2H), 6.59 (s, 1H), 6.32 (s, 2H), 5.09 (s, 1H), 3.46 (d, J=25.3 Hz, 4H), 2.99 (s, 2H), 2.81 (s, 2H), 1.91 (s, 2H).

### Example 21

### 2-{[4-(1-Methylpiperidin-4-yl)phenyl]amino}-8-phenyl-5-[2-(triisopropylsilyl)ethynyl]pyrido[2,3-d]pyrimidin-7-one

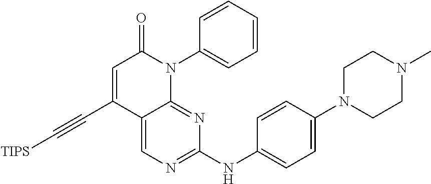

General procedure 13 was applied to 2-methanesulfonyl-8-phenyl-5-[2-(triisopropylsilyl)ethynyl]pyrido[2,3-d]pyrimidin-7-one (300 mg, 0.623 mmol) with trifluoroacetic acid (142 mg, 1.25 mmol) and 4-(1-methylpiperidin-4-yl)aniline in 2-butanol (5.0 mL). The reaction mixture was stirred at 100° C. for 5 h. The crude material was purified by reverse phase HPLC eluting with acetonitrile (5-85%) in water (0.4% trifluoroacetic acid) to yield the title compound as an orange solid (170 mg, 46%).

(ES, m/z): [M+H]⁺=592.3

### 5-Ethynyl-2-{[4-(1-methylpiperidin-4-yl)phenyl]amino}-8-phenylpyrido[2,3-d]pyrimidin-7-one

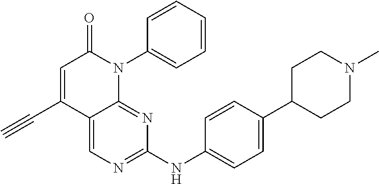 [SMILES: C#Cc1cc(=O)n(-c2ccccc2)c2nc(Nc3ccc(C4CCN(C)CC4)cc3)ncc12]

General procedure 15 was applied to 2-{[4-(1-methylpiperidin-4-yl)phenyl]amino}-8-phenyl-5-[2-(triisopropylsilyl)ethynyl]pyrido[2,3-d]pyrimidin-7-one (160 mg, 0.270 mmol) with potassium fluoride (314 mg, 5.40 mmol) in methanol (4.0 mL) and THF (4.0 mL). The reaction mixture was stirred at 70° C. for 2 h. The precipitated solids were collected by filtration and washed with water (10 mL) to yield the title compound as a light yellow solid (34.3 mg, 29%).

(ES, m/z): [M+H]⁺=436.2.

¹H NMR (400 MHz, DMSO-d₆) δ 10.24-9.99 (m, 1H), 8.82 (s, 1H), 7.63-7.56 (m, 3H), 7.37-7.33 (m, 2H), 7.18 (d, J=8.1 Hz, 2H), 6.81 (d, J=8.2 Hz, 2H), 6.67 (s, 1H), 5.12 (s, 1H), 2.93-2.80 (m, 2H), 2.38-2.28 (m, 1H), 2.21 (s, 3H), 2.05-1.92 (m, 2H), 1.69-1.50 (m, 4H).

### Example 22

### tert-Butyl 4-[4-({7-oxo-8-phenyl-5-[2-(triisopropylsilyl)ethynyl]pyrido[2,3-d]pyrimidin-2-yl}amino)phenyl]piperidine-1-carboxylate

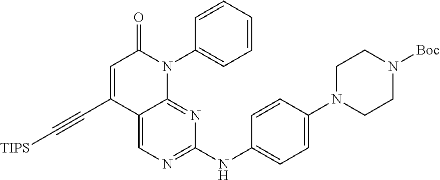

General procedure 13 was applied to 2-methanesulfonyl-8-phenyl-5-[2-(triisopropylsilyl)ethynyl]pyrido[2,3-d]pyrimidin-7-one (400 mg, 0.830 mmol) with tert-butyl 4-(4-aminophenyl)piperidine-1-carboxylate (275 mg, 0.996 mmol) and trifluoroacetic acid (189 mg, 1.66 mmol) in 2-butanol (4.0 mL). The reaction was stirred for 1 h at 100° C. The residue was purified by reverse phase flash column chromatography eluting with acetonitrile (30-60%) in water (0.1% formic acid) to yield the title compound as a yellow solid (600 mg, >100%).

(ES, m/z): [M+H]⁺=678.4.

### 8-Phenyl-2-{[4-(piperidin-4-yl)phenyl]amino}-5-[2-(triisopropylsilyl)ethynyl]pyrido[2,3-d]pyrimidin-7-one

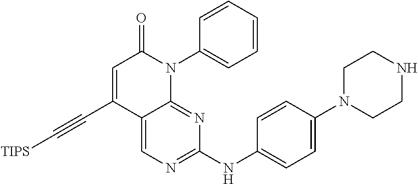

A solution of tert-butyl 4-[4-({7-oxo-8-phenyl-5-[2-(triisopropylsilyl)ethynyl]pyrido[2,3-d]pyrimidin-2-yl}amino)phenyl]piperidine-1-carboxylate (600 mg, 0.885 mmol, 1.0 eq.) in hydrogen chloride in methanol (0.60 mL) in methanol (6.0 mL) was stirred for 4 h at room temperature. The resulting mixture was concentrated under reduced pressure and purified by reverse phase flash column chromatography eluting with acetonitrile (10-50%) in water (0.1% formic acid) to yield the title compound as a yellow solid (210 mg, 41%).

(ES, m/z): [M+H]⁺=578.3.

### 5-Ethynyl-8-phenyl-2-{[4-(piperidin-4-yl)phenyl]amino}pyrido[2,3-d]pyrimidin-7-one

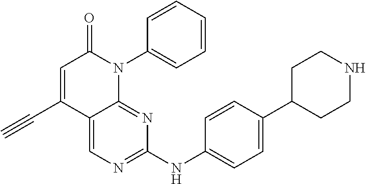 [SMILES: C#Cc1cc(=O)n(-c2ccccc2)c2nc(Nc3ccc(C4CCNCC4)cc3)ncc12]

General procedure 15 was applied to 8-phenyl-2-{[4-(piperidin-4-yl)phenyl]amino}-5-[2-(triisopropylsilyl)ethynyl]pyrido[2,3-d]pyrimidin-7-one (210 mg, 0.363 mmol) with potassium fluoride (422 mg, 7.26 mmol) in THF (2.0 mL), water (50 μL) and methanol (1.0 mL). The resulting mixture was stirred for 2 h at 80° C. and purified by reverse phase flash column chromatography eluting with acetonitrile (30-60%) in water (0.1% formic acid) to yield the title compound as a yellow solid (6.80 mg, 4%).

(ES, m/z): [M+H]⁺=422.2

¹H NMR (300 MHz, DMSO-d₆) δ 10.16 (s, 1H), 8.84 (s, 1H), 7.60 (d, J=6.8 Hz, 3H), 7.40-7.34 (m, 2H), 7.23 (d, J=7.3 Hz, 2H), 6.83 (d, J=8.2 Hz, 2H), 6.69 (s, 1H), 5.13 (s, 1H), 2.92 (s, 3H), 2.67 (s, 2H), 1.73 (d, J=28.4 Hz, 4H).

### Example 23

### 2-({4-[4-(4-Methylpiperazin-1-yl)piperidin-1-yl]phenyl}amino)-8-phenyl-5-[2-(triisopropylsilyl)ethynyl]pyrido[2,3-d]pyrimidin-7-one

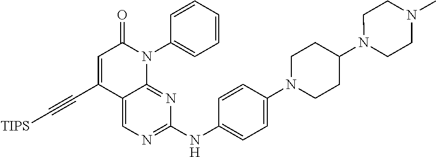

General procedure 13 was applied to 2-methanesulfonyl-8-phenyl-5-[2-(triisopropylsilyl)ethynyl]pyrido[2,3-d]pyrimidin-7-one (500 mg, 1.04 mmol), 4-(4-(4-methylpiperazin-1-yl)piperidin-1-yl)aniline in 2-butanol (5.0 mL). The resulting solution was stirred for 3 h at 100° C., diluted with methanol (10 mL) and directly applied onto a reverse phase column, eluting with acetonitrile (70-100%) in water (0.1% trifluoroacetic acid). The mixture was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a red solid (270 mg, 38%).

(ES, m/z): [M+H]⁺=676.4

### 5-Ethynyl-2-({4-[4-(4-methylpiperazin-1-yl)piperidin-1-yl]phenyl}amino)-8-phenylpyrido[2,3-d]pyrimidin-7-one

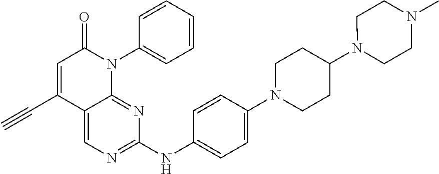 [SMILES: C#Cc1cc(=O)n(-c2ccccc2)c2nc(Nc3ccc(N4CCC(N5CCN(C)CC5)CC4)cc3)ncc12]

General procedure 15 was applied to 2-({4-[4-(4-methylpiperazin-1-yl)piperidin-1-yl]phenyl}amino)-8-phenyl-5-[2-(triisopropylsilyl)ethynyl]pyrido[2,3-d]pyrimidin-7-one (200 mg, 0.30 mmol) with potassium fluoride (172 mg, 2.96 mmol) in methanol (1.0 mL) and THF (0.50 mL). The resulting solution was stirred for 2 h at 50° C. and directly applied onto a reverse phase column, eluting with acetonitrile (20-100%) in water (0.1% formic acid). The desired fractions were collected and concentrated under reduced pressure to remove most of the acetonitrile and lyophilized to yield the title compound as an orange solid (34.0 mg, 22%).

(ES, m/z): [M+H]⁺=520.2

¹H NMR (300 MHz, DMSO-d₆) δ 9.94 (s, 1H), 8.74 (s, 1H), 7.58 (s, 3H), 7.34 (s, 2H), 7.10 (s, 2H), 6.58 (s, 3H), 3.53 (s, 2H), 2.74 (s, 10H), 2.43 (s, 3H), 1.85 (s, 2H), 1.55 (s, 2H), 1.17 (s, 22H).

### Example 24

### 1-(2-Bromo-4-nitrophenyl)-4-methylpiperazine

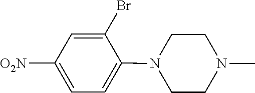 [SMILES: CN1CCN(c2ccc([N+](=O)[O-])cc2Br)CC1]

General procedure 11 was applied to 2-bromo-1-fluoro-4-nitrobenzene (5.00 g, 22.7 mmol), 1-methylpiperazine (2.73 g, 27.3 mmol) and potassium carbonate (7.54 g, 54.5 mmol) in DMF (50 mL). The crude material was purified by flash column chromatography eluting with ethyl acetate (0-75%) in 40-60 petroleum ether to yield the title compound as a light yellow solid (4.50 g, 66%).

(ES, m/z): [M+H]⁺=200.2

### 1-(2-Ethenyl-4-nitrophenyl)-4-methylpiperazine

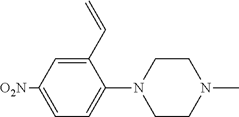 [SMILES: C=Cc1cc([N+](=O)[O-])ccc1N1CCN(C)CC1]

A solution of 1-(2-bromo-4-nitrophenyl)-4-methylpiperazine (1.00 g, 3.32 mmol, 1.0 eq.), [1,1′-bis(diphenylphosphino)ferrocene]dichloropalladium(II) (0.24 g, 0.332 mmol, 0.1 eq.), tributyl(ethenyl)stannane (1.37 g, 4.31 mmol, 1.3 eq.) in toluene (10 mL) was stirred for 2 h at 80° C. The resulting mixture was concentrated under reduced pressure and the crude material was purified by flash column chromatography, eluting with methanol (0-10%) in dichloromethane to yield the title compound as a light yellow solid (670 mg, 81%).

(ES, m/z): [M+H]⁺=248.1

### 3-Ethyl-4-(4-methylpiperazin-1-yl)aniline

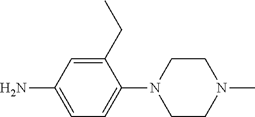 [SMILES: CCc1cc(N)ccc1N1CCN(C)CC1]

General procedure 12 was applied to 1-(2-ethenyl-4-nitrophenyl)-4-methylpiperazine (700 mg, 2.81 mmol) with palladium on carbon 5 wt. % (68.8 mg, 0.647 mmol) in methanol (7.0 mL) to yield the title compound as a dark grey solid (615 mg, 98%).

(ES, m/z): [M+H]⁺=220.1

### 2-{[3-Ethyl-4-(4-methylpiperazin-1-yl)phenyl]amino}-8-phenyl-5-[2-(triisopropylsilyl)ethynyl]pyrido[2,3-d]pyrimidin-7-one

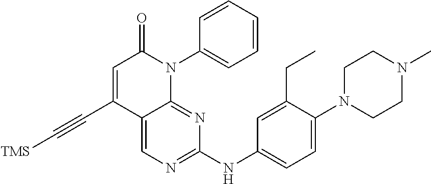 [SMILES: CCc1cc(Nc2ncc3c(C#C[Si](C)(C)C)cc(=O)n(-c4ccccc4)c3n2)ccc1N1CCN(C)CC1]

General procedure 13 was applied to 2-methanesulfonyl-8-phenyl-5-[2-(triisopropylsilyl)ethynyl]pyrido[2,3-d]pyrimidin-7-one (600 mg, 1.25 mmol) with 3-ethyl-4-(4-methylpiperazin-1-yl)aniline (219 mg, 0.997 mmol) and trifluoroacetic acid (355 mg, 3.12 mmol) in 2-butanol (12.0 mL). The reaction mixture was stirred at 110° C. for 24 h and directly applied onto a reverse phase column, eluting with acetonitrile (10-100%) in water (0.1% trifluoroacetic acid) to yield the title compound as a yellow solid (225 mg, 29%).

(ES, m/z): [M+H]⁺=621.5

### 2-{[3-Ethyl-4-(4-methylpiperazin-1-yl)phenyl]amino}-5-ethynyl-8-phenylpyrido[2,3-d]pyrimidin-7-one

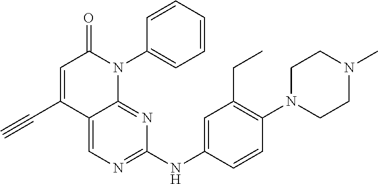 [SMILES: C#Cc1cc(=O)n(-c2ccccc2)c2nc(Nc3ccc(N4CCN(C)CC4)c(CC)c3)ncc12]

General procedure 15 was applied to 2-{[3-ethyl-4-(4-methylpiperazin-1-yl)phenyl]amino}-8-phenyl-5-[2-(triisopropylsilyl)ethynyl]pyrido[2,3-d]pyrimidin-7-one (300 mg, 0.478 mmol) with potassium fluoride (277 mg, 4.78 mmol) in methanol (1.5 mL), THF (3.0 mL) and water (0.10 mL). The reaction mixture was stirred at room temperature for 2 h and applied directly onto a reverse phase column, eluting with acetonitrile (10-100%) in water (0.1% formic acid). The desired fractions were collected and concentrated under reduced pressure to remove most of the acetonitrile then lyophilized to yield the title compound as a yellow solid (67 mg, 29%).

(ES, m/z): [M+H]⁺=465.3

¹H NMR (400 MHz, DMSO-d6) δ 10.03 (s, 1H), 8.81 (s, 1H), 7.57 (d, J=9.8 Hz, 3H), 7.34 (d, J=7.3 Hz), 7.10 (2H, d, J=26.9 Hz, 2H), 6.65 (s, 2H), 5.11 (s, 1H), 2.71 (s, 5H), 2.45-2.31 (m, 4H), 2.28 (s, 4H), 1.03 (d, J=7.8 Hz, 3H).

### Example 25

### 2-[(1-Methylpyrazol-4-yl)amino]-8-phenyl-5-[2-(triisopropylsilyl)ethynyl]pyrido[2,3-d]pyrimidin-7-one

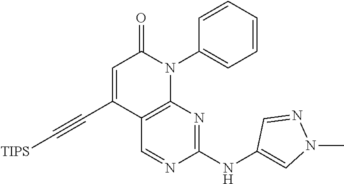

General procedure 13 was applied to 2-methanesulfonyl-8-phenyl-5-[2-(triisopropylsilyl)ethynyl]pyrido[2,3-d]pyrimidin-7-one (450 mg, 0.906 mmol) with 1-methylpyrazol-4-amine (132 mg, 1.36 mmol) and trifluoroacetic acid (258 mg, 2.26 mmol) in 2-butanol (10 mL). The reaction mixture was stirred at 110° C. for 2 h and directly applied onto a reverse phase column, eluting with acetonitrile (10-100%) in water (0.1% formic acid) to yield the title compound as a yellow solid (280 mg, 56%).

(ES, m/z): [M+H]⁺=499.3.

### 5-Ethynyl-2-[(1-methylpyrazol-4-yl)amino]-8-phenylpyrido[2,3-d]pyrimidin-7-one

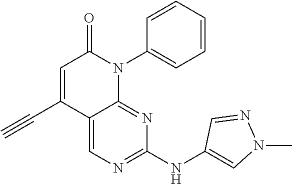 [SMILES: C#Cc1cc(=O)n(-c2ccccc2)c2nc(Nc3cnn(C)c3)ncc12]

General procedure 15 was applied to 2-[(1-methylpyrazol-4-yl)amino]-8-phenyl-5-[2-(triisopropylsilyl)ethynyl]pyrido[2,3-d]pyrimidin-7-one (280 mg, 0.512 mmol) with potassium fluoride (297 mg, 5.12 mmol) in THF (2.8 mL), methanol (1.4 mL) and water (0.19 mL). The reaction mixture was stirred at room temperature for 2 h and directly applied onto a reverse phase column, eluting with acetonitrile (10-100%) in water (0.1% formic acid). The desired fractions were collected and concentrated under reduced pressure to remove most of the acetonitrile then lyophilized to yield the title compound as a yellow solid (25.0 mg, 14%).

(ES, m/z): [M+H]⁺=343.1.

¹H NMR (400 MHz, DMSO-d₆) δ 10.17 (s, 1H), 8.80 (s, 1H), 7.65 (dd, J=12.0, 7.1 Hz, 3H), 7.39 (d, J=7.2 Hz, 2H), 7.12 (s, 1H), 6.65 (d, J=14.8 Hz, 2H), 4.96 (s, 1H), 3.54 (s, 3H).

### Example 26

### tert-Butyl 7-({7-oxo-8-phenyl-5-[2-(triisopropylsilyl)ethynyl]pyrido[2,3-d]pyrimidin-2-yl}amino)-1,2,4,5-tetrahydro-3-benzazepine-3-carboxylate

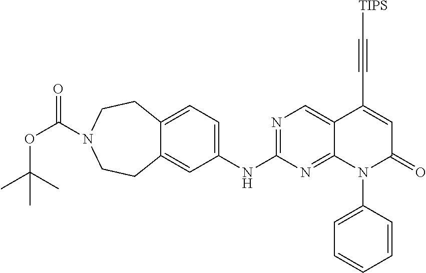

General procedure 13 was applied to 2-methanesulfonyl-8-phenyl-5-[2-(triisopropylsilyl)ethynyl]pyrido[2,3-d]pyrimidin-7-one (400 mg, 0.830 mmol) with tert-butyl 7-amino-1,2,4,5-tetrahydro-3-benzazepine-3-carboxylate (261 mg, 0.996 mmol) and trifluoroacetic acid (189 mg, 1.66 mmol) in 2-butanol (4.0 mL). The resulting solution was stirred for 3 h at 100° C. and concentrated under reduced pressure to yield the title compound as an orange solid (400 mg, 73%).

(ES, m/z): [M+H]⁺=664.4.

### 8-Phenyl-2-(2,3,4,5-tetrahydro-1H-3-benzazepin-7-ylamino)-5-[2-(triisopropylsilyl)ethynyl]pyrido[2,3-d]pyrimidin-7-one

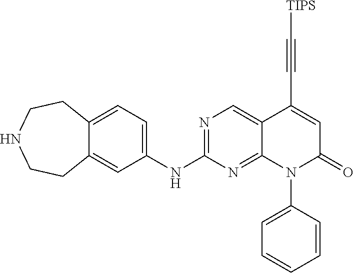

To a solution of tert-butyl 7-({7-oxo-8-phenyl-5-[2-(triisopropylsilyl)ethynyl]pyrido[2,3-d]pyrimidin-2-yl}amino)-1,2,4,5-tetrahydro-3-benzazepine-3-carboxylate (400 mg, 0.602 mmol, 1.00 eq.) with hydrogen chloride in methanol (4.0 mL) was stirred for 8 h at 30° C. The reaction mixture was applied onto a reverse phase column directly, eluting with acetonitrile (20-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (270 mg, 79%).

(ES, m/z): [M+H]⁺=564.3.

### 5-ethynyl-8-phenyl-2-(2,3,4,5-tetrahydro-1H-3-benzazepin-7-ylamino)pyrido [2,3-d]pyrimidin-7-one

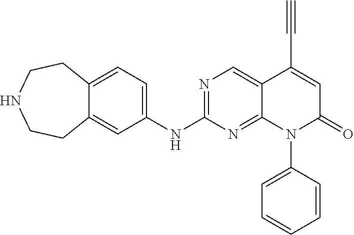 [SMILES: C#Cc1cc(=O)n(-c2ccccc2)c2nc(Nc3ccc4c(c3)CCNCC4)ncc12]

General procedure 15 was applied to 8-phenyl-2-(2,3,4,5-tetrahydro-1H-3-benzazepin-7-ylamino)-5-[2-(triisopropylsilyl)ethynyl]pyrido[2,3-d]pyrimidin-7-one (150 mg, 0.266 mmol) with potassium fluoride (154 mg, 2.66 mmol) in THF (2.0 mL), methanol (2.0 mL) and water (0.50 mL). The resulting solution was stirred for 2 h at 70° C. and applied onto a reverse phase column, eluting with acetonitrile (20-100%) in water (0.1% formic acid). The desired fractions were collected and concentrated under reduced pressure to remove most of the acetonitrile then lyophilized to yield the title compound as a yellow solid (17.3 mg, 16%).

(ES, m/z): [M+H]⁺=408.2.

¹H-NMR (300 MHz, DMSO-d₆): δ 10.12 (s, 1H), 8.84 (s, 1H), 7.59 (d, J=7.5 Hz, 4H), 7.37 (d, J=6.6 Hz, 4H), 7.20-7.12 (m, 2H), 7.03-7.00 (m, 1H), 6.78-6.74 (m, 1H), 6.68 (s, 1H), 5.12 (s, 1H), 2.89-2.83 (m, 6H), 2.73-2.60 (m, 2H).

### Example 27

### tert-Butyl 2-methyl-4-[4-({7-oxo-8-phenyl-5-[2-(triisopropylsilyl)ethynyl]pyrido[2,3-d]pyrimidin-2-yl}amino)phenyl]piperazine-1-carboxylate

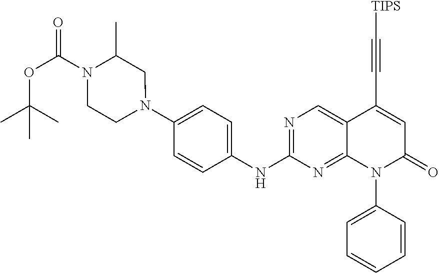

General procedure 13 was applied to 2-methanesulfonyl-8-phenyl-5-[2-(triisopropylsilyl)ethynyl]pyrido[2,3-d]pyrimidin-7-one (400 mg, 0.830 mmol) with trifluoroacetic acid (284 mg, 2.49 mmol), tert-butyl 4-(4-aminophenyl)-2-methylpiperazine-1-carboxylate (290 mg, 0.996 mmol) in 2-butanol (4.0 mL). The resulting solution was stirred for 3 h at 100° C. and concentrated under reduced pressure to yield the title compound as an orange solid (500 mg, 87%).

(ES, m/z): [M+H]⁺=693.4.

### 2-{[4-(3-Methylpiperazin-1-yl)phenyl]amino}-8-phenyl-5-[2-(triisopropylsilyl)ethynyl]pyrido[2,3-d]pyrimidin-7-one

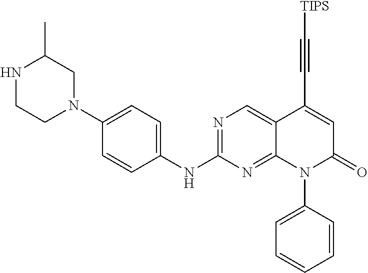

A solution of tert-butyl 2-methyl-4-[4-({7-oxo-8-phenyl-5-[2-(triisopropylsilyl)ethynyl]pyrido[2,3-d]pyrimidin-2-yl}amino)phenyl]piperazine-1-carboxylate (500 mg, 0.722 mmol, 1.0 eq.) with hydrogen chloride in methanol (5.0 mL) was stirred for 8 h at 30° C. The reaction mixture was directly applied onto a reverse phase column, eluting with acetonitrile (20-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (250 mg, 58%).

(ES, m/z): [M+H]⁺=593.3.

### 5-Ethynyl-2-{[4-(3-methylpiperazin-1-yl)phenyl]amino}-8-phenylpyrido[2,3-d]pyrimidin-7-one

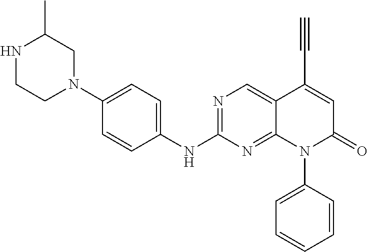 [SMILES: C#Cc1cc(=O)n(-c2ccccc2)c2nc(Nc3ccc(N4CCNC(C)C4)cc3)ncc12]

General procedure 15 was applied to 2-{[4-(3-methylpiperazin-1-yl)phenyl]amino}-8-phenyl-5-[2-(triisopropylsilyl)ethynyl]pyrido[2,3-d]pyrimidin-7-one (250 mg, 0.422 mmol) with potassium fluoride (245 mg, 4.22 mmol) in THF (3.0 mL), methanol (3.0 mL) and water (0.50 mL). The resulting solution was stirred for 2 h at 70° C. then directly applied onto a reverse phase column, eluting with acetonitrile (20-100%) in water (0.1% formic acid). The desired fractions were collected and concentrated under reduced pressure to remove most of the acetonitrile then lyophilized to yield the title compound as an orange solid (86.6 mg, 47%).

(ES, m/z): [M+H]⁺=437.1.

¹H-NMR (300 MHz, DMSO-d₆): δ 10.01 (s, 1H), 8.78 (s, 1H), 7.60-7.58 (m, 3H), 7.35 (d, J=6.6 Hz, 2H), 7.13 (d, J=5.1 Hz, 2H), 6.63-6.55 (m, 3H), 5.10 (s, 1H), 3.40 (d, J=9.0 Hz, 2H), 3.11-3.07 (m, 1H), 2.97-2.93 (m, 2H), 2.60-2.59 (m, 1H), 2.27 (d, J=5.1 Hz, 1H), 1.12 (s, 3H).

### Example 28

### 2-((4-(4-(3-(Hydroxymethyl)azetidin-1-yl)piperidin-1-yl)phenyl)amino)-8-phenyl-5-((triisopropylsilyl)ethynyl)pyrido[2,3-d]pyrimidin-7(8H)-one

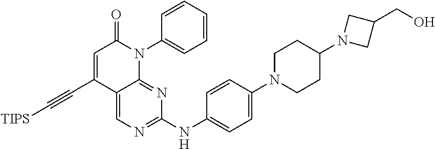

General procedure 13 was applied to 2-methanesulfonyl-8-phenyl-5-[2-(triisopropylsilyl)ethynyl]pyrido[2,3-d]pyrimidin-7-one (300 mg, 0.623 mmol) with {1-[1-(4-aminophenyl)piperidin-4-yl]azetidin-3-yl}methanol (163 mg, 0.623 mmol) and trifluoroacetic acid (142 mg, 1.25 mmol) in 2-butanol (10 mL). The reaction mixture was stirred at 110° C. for 3 h then purified by reverse phase HPLC eluting with acetonitrile (5-80%) in water (0.4% formic acid) to give the title compound as a brown solid (180 mg, 57%).

(ES, m/z): [M+H]⁺=663.5.

### 5-Ethynyl-2-[(4-{4-[1-(hydroxymethyl)azetidin-3-yl]piperidin-1-yl}phenyl)amino]-8-phenylpyrido[2,3-d]pyrimidin-7-one

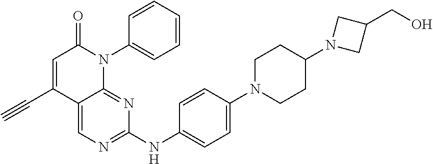 [SMILES: C#Cc1cc(=O)n(-c2ccccc2)c2nc(Nc3ccc(N4CCC(N5CC(CO)C5)CC4)cc3)ncc12]

General procedure 15 was applied to 2-[(4-{4-[1-(hydroxymethyl)azetidin-3-yl]piperidin-1-yl}phenyl)amino]-8-phenyl-5-[2-(triisopropylsilyl)ethynyl]pyrido[2,3-d]pyrimidin-7-one (170 mg, 0.256 mmol) with potassium fluoride (29.8 mg, 0.512 mmol) in DMF (10 mL) and water (9.2 μL). The reaction mixture was stirred at 45° C. for 1.5 h and purified by reverse phase HPLC eluting with acetonitrile (5-50%) in water (0.4% formic acid), to yield the title compound as an orange solid (51.0 mg, 38%).

(ES, m/z) [M+H]⁺=507.4.

¹H NMR (400 MHz, DMSO-d₆) δ 10.02 (s, 1H), 8.78 (s, 1H), 8.23 (s, 1H), 7.62-7.56 (m, 3H), 7.37-7.31 (m, 2H), 7.09 (d, J=8.2 Hz, 2H), 6.63 (s, 1H), 6.51 (d, J=8.6 Hz, 2H), 5.10 (s, 1H), 3.52-3.33 (m, 6H), 3.12 (t, J=7.1 Hz, 2H), 2.69-2.53 (m, 3H), 2.45-2.31 (m, 1H), 1.83-1.69 (m, 2H), 1.34-1.16 (m, 2H).

### Example 29

### tert-Butyl 4-[3-(dimethylamino)azetidin-1-yl]piperidine-1-carboxylate

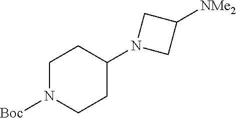 [SMILES: CN(C)C1CN(C2CCN(C(=O)OC(C)(C)C)CC2)C1]

A solution of N,N-dimethylazetidin-3-amine (3.00 g, 29.9 mmol, 1.0 eq.), tert-butyl 4-oxopiperidine-1-carboxylate (11.9 g, 59.9 mmol, 2.0 eq.) and acetic acid (900 mg, 14.9 mmol, 0.5 eq.) in dichloromethane (15 mL) and methanol (15 mL) was stirred for 30 minutes at room temperature, then sodium cyanoborohydride (3.76 g, 59.9 mmol, 2.0 eq.) was added. The resulting solution was stirred overnight at room temperature and directly applied onto a reverse phase column, eluting with acetonitrile (90-100%) in water (0.1% formic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a white solid (2.50 g, 29%).

(ES, m/z): [M+H]⁺=284.2.

### N,N-Dimethyl-1-(piperidin-4-yl)azetidin-3-amine

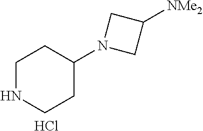 [SMILES: CN(C)C1CN(C2CCNCC2)C1 ; Cl]

A solution of tert-butyl 4-[3-(dimethylamino)azetidin-1-yl]piperidine-1-carboxylate (2.50 g, 8.82 mmol, 1.0 eq.), hydrogen chloride in methanol (25 mL). The resulting solution was stirred for 3 h at 30° C. then concentrated under reduced pressure to yield the title compound as a white solid (2.00 g, 124%).

(ES, m/z): [M+H]⁺=184.2.

### N,N-Dimethyl-1-[1-(4-nitrophenyl)piperidin-4-yl]azetidin-3-amine

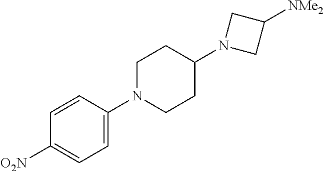 [SMILES: CN(C)C1CN(C2CCN(c3ccc([N+](=O)[O-])cc3)CC2)C1]

General procedure 11 was applied to N,N-dimethyl-1-(piperidin-4-yl)azetidin-3-amine (1.00 g, 5.46 mmol, 1.00 eq.), 4-fluoronitrobenzene (920 mg, 6.55 mmol, 1.2 eq.) and potassium carbonate (1.51 g, 10.9 mmol, 2.0 eq.) in DMF (10 mL). The crude material was purified by flash column chromatography eluting with ethyl acetate (20%) in 40-60 petroleum ether to yield the title compound as an orange solid (1.00 g, 60%).

(ES, m/z): [M+H]⁺=305.2.

### 1-[1-(4-Aminophenyl)piperidin-4-yl]-N,N-dimethylazetidin-3-amine

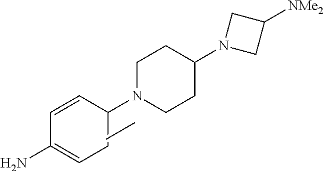

General procedure 12 was applied to N,N-dimethyl-1-[1-(4-nitrophenyl)piperidin-4-yl]azetidin-3-amine (1.00 g, 3.28 mmol) and palladium on carbon 5 wt. % (200 mg, 1.88 mmol) in methanol (10 mL) to yield the title compound as a red solid (600 mg, 66%).

(ES, m/z): [M+H]⁺=275.2.

### 2-[(4-{4-[3-(Dimethylamino)azetidin-1-yl]piperidin-1-yl}phenyl)amino]-8-phenyl-5-[2-(triisopropylsilyl)ethynyl]pyrido[2,3-d]pyrimidin-7-one

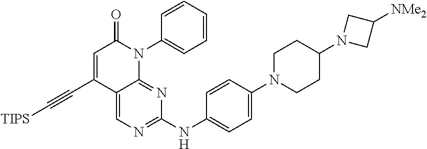

General procedure 13 was applied to 2-methanesulfonyl-8-phenyl-5-[2-(triisopropylsilyl)ethynyl]pyrido[2,3-d]pyrimidin-7-one (500 mg, 1.04 mmol) with trifluoroacetic acid (236 mg, 2.07 mmol) and 1-[1-(4-aminophenyl)piperidin-4-yl]-N,N-dimethylazetidin-3-amine (342 mg, 1.25 mmol) in 2-butanol (5.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a red solid (250 mg, 36%).

(ES, m/z): [M+H]⁺=676.4.

### 2-[(4-{4-[3-(Dimethylamino)azetidin-1-yl]piperidin-1-yl}phenyl)amino]-5-ethynyl-8-phenylpyrido[2,3-d]pyrimidin-7-one

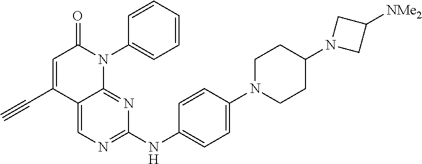 [SMILES: C#Cc1cc(=O)n(-c2ccccc2)c2nc(Nc3ccc(N4CCC(N5CC(N(C)C)C5)CC4)cc3)ncc12]

General procedure 15 was applied to 2-[(4-{4-[3-(dimethylamino)azetidin-1-yl]piperidin-1-yl}phenyl)amino]-8-phenyl-5-[2-(triisopropylsilyl)ethynyl]pyrido[2,3-d]pyrimidin-7-one (250 mg, 0.370 mmol) with potassium fluoride (215 mg, 3.70 mmol) in THF (3.0 mL), methanol (3.0 mL) and water (0.50 mL). The resulting solution was stirred for 2 h at 70° C. and applied onto a reverse phase column, eluting with acetonitrile (20-100%) in water (0.1% formic acid). The desired fractions were collected and concentrated under reduced pressure to remove most of the acetonitrile then lyophilized to yield the title compound as an orange solid (115 mg, 60%).

(ES, m/z): [M+H]⁺=520.5.

¹H-NMR (300 MHz, DMSO-d₆) δ 10.01 (s, 1H), 8.78 (s, 1H), 7.60-7.58 (m, 3H), 7.35-7.33 (m, 2H), 7.12 (d, J=4.9 Hz, 2H), 6.63-6.53 (m, 3H), 5.10 (s, 1H), 3.50-3.42 (m, 2H), 3.40-3.38 (m, 2H), 2.96-2.94 (m, 2H), 2.84-2.80 (m, 1H), 2.61-2.57 (m, 2H), 2.37-2.28 (m, 1H), 2.06 (s, 6H), 1.79-1.69 (m, 2H), 1.29-1.26 (m, 2H).

### Example 30

### (3-Fluoroazetidin-3-yl)methanol

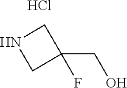

A solution of tert-butyl 3-fluoro-3-(hydroxymethyl)azetidine-1-carboxylate (1.40 g, 6.82 mmol, 1.0 eq.) with hydrogen chloride in methanol (10 mL) was stirred for 5 h at 40° C. The resulting mixture was concentrated under reduced pressure to yield the title compound as a white solid (1.20 g, 167%).

(ES, m/z): [M+H]⁺=106.0.

### tert-Butyl 4-[3-fluoro-3-(hydroxymethyl)azetidin-1-yl]piperidine-1-carboxylate

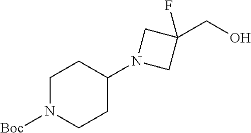 [SMILES: CC(C)(C)OC(=O)N1CCC(N2CC(F)(CO)C2)CC1]

To a solution of (3-fluoroazetidin-3-yl) methanol (2.20 g, 20.9 mmol, 1.0 eq.) in methanol (20 mL), dichloromethane (20 mL) and acetic acid (1.0 mL) was added tert-butyl 4-oxopiperidine-1-carboxylate (8.34 g, 41.8 mmol, 2.0 eq.). The reaction mixture was stirred at 30° C. for 1 h and sodium cyanoborohydride (2.63 g, 41.8 mmol, 2.0 eq.) was added. After stirring for 16 h at room temperature, the reaction was quenched with saturated aqueous sodium carbonate, the pH was adjusted to 7 and extracted with dichloromethane (3×500 mL). The combined organic layers were washed with brine (100 mL), dried (Na₂SO₄) and concentrated under reduced pressure. The crude material was purified by flash column chromatography eluting with methanol (0-20%) in dichloromethane to afford the title compound as a white solid (1.60 g, 26%).

(ES, m/z): [M+H]⁺=289.1.

### (3-Fluoroazetidin-3-yl)methanol

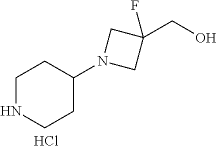 [SMILES: OCC1(F)CN(C2CCNCC2)C1 ; Cl]

A solution of tert-butyl 3-fluoro-3-(hydroxymethyl)azetidine-1-carboxylate (1.40 g, 6.82 mmol, 1.0 eq.) with hydrogen chloride in methanol (10 mL) was stirred for 5 h at 40° C. The resulting mixture was concentrated under reduced pressure to yield the title compound as a white solid (1.20 g, 167%).

(ES, m/z): [M+H]⁺=189.1.

### (3-Fluoro-1-[1-(4-nitrophenyl)piperidin-4-yl]azetidin-3-yl}methanol

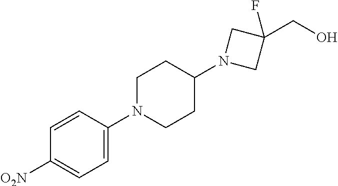 [SMILES: O=[N+]([O-])c1ccc(N2CCC(N3CC(F)(CO)C3)CC2)cc1]

General procedure 11 was applied to (3-fluoro-1-(piperidin-4-yl)azetidin-3-yl]methanol hydrochloride (1.20 g, 5.34 mmol) with 4-fluoronitrobenzene (900 mg, 6.41 mmol) and potassium carbonate (1.48 g, 10.7 mmol) in DMF (10 mL). The residue was triturated with 40-60 petroleum ether:ethyl acetate (60 mL, 1:1) and filtered, washed with 40-60 petroleum ether and dried under vacuum to yield the title compound as a yellow solid. (600 mg, 36%).

(ES, m/z): [M+H]⁺=310.1.

### (1-[1-(4-Aminophenyl)piperidin-4-yl]-3-fluoroazetidin-3-yl)methanol

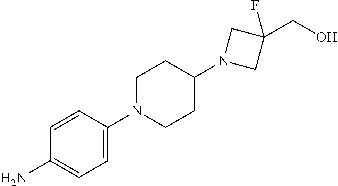 [SMILES: Nc1ccc(N2CCC(N3CC(F)(CO)C3)CC2)cc1]

General procedure 12 was applied to (3-fluoro-1-[1-(4-nitrophenyl)piperidin-4-yl]azetidin-3-yl}methanol (700 mg, 2.26 mmol) with palladium on carbon 5 wt. % (10.0 mg, 94.0 μmol) in methanol (10 mL) to yield the title compound as a grey solid (500 mg, 79%).

(ES, m/z): [M+H]⁺=280.1.

### 2-[(4-{4-[3-Fluoro-3-(hydroxymethyl)azetidin-1-yl]piperidin-1-yl}phenyl)amino]-8-phenyl-5-[2-(triisopropylsilyl)ethynyl]pyrido[2,3-d]pyrimidin-7-one

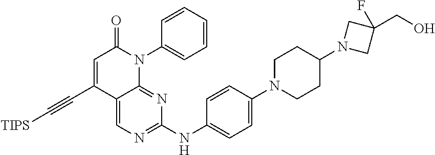

General procedure 13 was applied to 2-methanesulfonyl-8-phenyl-5-[2-(triisopropylsilyl)ethynyl]pyrido[2,3-d]pyrimidin-7-one (500 mg, 1.04 mmol) with 4-(4-(3-ethyl-3-fluoroazetidin-1-yl)piperidin-1-yl)aniline and trifluoroacetic acid in 2-butanol (5.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (287 mg, 41%).

(ES, m/z): [M+H]⁺=681.3.

### 5-Ethynyl-2-[(4-{4-[3-fluoro-3-(hydroxymethyl)azetidin-1-yl]piperidin-1-yl}phenyl)amino]-8-phenylpyrido[2,3-d]pyrimidin-7-one

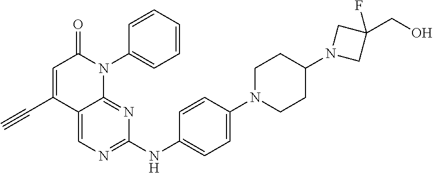 [SMILES: C#Cc1cc(=O)n(-c2ccccc2)c2nc(Nc3ccc(N4CCC(N5CC(F)(CO)C5)CC4)cc3)ncc12]

General procedure 15 was applied to 2-[(4-{4-[3-fluoro-3-(hydroxymethyl)azetidin-1-yl]piperidin-1-yl}phenyl)amino]-8-phenyl-5-[2-(triisopropylsilyl)ethynyl]pyrido[2,3-d]pyrimidin-7-one (287 mg, 0.421 mmol) with potassium fluoride (232 mg, 4.01 mmol) in THF (1.0 mL) and methanol (1.0 mL). The resulting solution was stirred for 2 h at 50° C. and applied onto a reverse phase column, eluting with acetonitrile (20-100%) in water (0.1% formic acid). The desired fractions were collected and concentrated under reduced pressure to remove most of the acetonitrile then lyophilized to yield the title compound as a yellow solid.

(ES, m/z): [M+H]⁺=525.3.

¹H-NMR (300 MHz, DMSO-d₆) δ 9.99 (s, 1H), 8.78 (s, 1H), 7.60 (s, 3H), 7.33 (s, 2H), 7.12 (s, 2H), 6.59 (d, J=25.3 Hz, 3H), 5.10 (s, 1H), 3.64 (d, J=25.3 Hz, 3H), 3.38 (s, 5H), 3.07 (d, J=24.2 Hz, 3H), 2.64 (s, 2H), 2.19 (s, 1H), 1.73 (s, 2H), 1.25 (s, 2H).

### Example 31

### 2-(4-Methylpiperazin-1-yl)-5-nitrobenzaldehyde

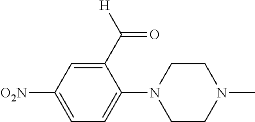 [SMILES: CN1CCN(c2ccc([N+](=O)[O-])cc2C=O)CC1]

General procedure 11 was applied to 2-fluoro-5-nitrobenzaldehyde (500 mg, 2.96 mmol) with 1-methylpiperazine (355 mg, 3.55 mmol) and potassium carbonate (817 mg, 5.91 mmol) in DMF (6.0 mL). The residue was triturated with 40-60 petroleum ether:ethyl acetate (100 mL, 1:1), filtered and washed with 40-60 petroleum ether and then dried under vacuum to yield the title compound as a yellow solid (729 mg, 99%).

(ES, m/z): [M+H]⁺=250.1.

### [5-(4-Methylpiperazin-1-yl)-5-nitrophenyl]methanol

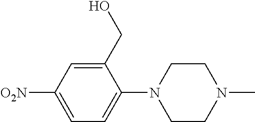 [SMILES: CN1CCN(c2ccc([N+](=O)[O-])cc2CO)CC1]

To a stirred solution of 2-(4-methylpiperazin-1-yl)-5-nitrobenzaldehyde (3.00 g, 12.0 mmol, 1.0 eq.) in methanol (30 mL) was added sodium borohydride (680 mg, 18.1 mmol, 1.5 eq.) in portions at 0° C. and stirred for 2 h at room temperature. The resulting mixture was concentrated under reduced pressure then quenched with saturated aqueous ammonium chloride solution and extracted with dichloromethane (3×100 mL). The combined organic layers were washed with brine (100 mL), dried (Na₂SO₄) and concentrated under reduced pressure. The crude mixture was washed with 40-60 petroleum ether:ethyl acetate (30 mL, 3:1) to yield the title compound as a yellow solid (2.62 g, 87%).

(ES, m/z): [M+H]⁺=252.1

### [5-Amino-2-(4-methylpiperazin-1-yl)phenyl]methanol

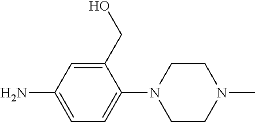 [SMILES: CN1CCN(c2ccc(N)cc2CO)CC1]

General procedure 12 was applied to [2-(4-methylpiperazin-1-yl)-5-nitrophenyl]methanol (900 mg, 3.58 mmol) with palladium on carbon 5 wt. % (100 mg, 0.940 mmol) in methanol (10 mL) to yield the title compound as a black oil (700 mg, 88%).

(ES, m/z): [M+H]⁺=222.1

### 2-{[3-(Hydroxymethyl)-4-(4-methylpiperazin-1-yl)phenyl]amino}-8-phenyl-5-[2-(triisopropylsilyl)ethynyl]pyrido[2,3-d]pyrimidin-7-one

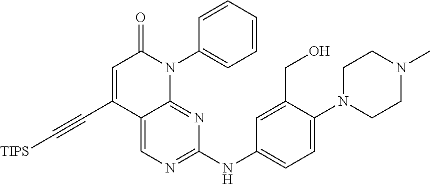

General procedure 13 applied to 2-methanesulfonyl-8-phenyl-5-[2-(triisopropylsilyl)ethynyl]pyrido[2,3-d]pyrimidin-7-one (500 mg, 1.04 mmol) with [5-amino-2-(4-methylpiperazin-1-yl)phenyl]methanol (276 mg, 1.25 mmol) in 2-butanol (5.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a red solid (269 mg, 42%).

(ES, m/z): [M+H]⁺=623.3.

### 5-Ethynyl-2-{[3-(hydroxymethyl)-4-(4-methylpiperazin-1-yl)phenyl]amino}-8-phenylpyrido[2,3-d]pyrimidin-7-one

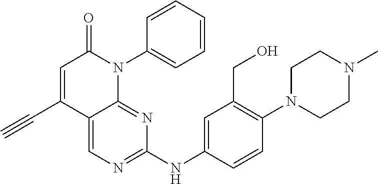 [SMILES: C#Cc1cc(=O)n(-c2ccccc2)c2nc(Nc3ccc(N4CCN(C)CC4)c(CO)c3)ncc12]

General procedure 15 was applied to 2-{[3-(hydroxymethyl)-4-(4-methylpiperazin-1-yl)phenyl]amino}-8-phenyl-5-[2-(triisopropylsilyl)ethynyl]pyrido[2,3-d]pyrimidin-7-one (269 mg, 0.432 mmol) with potassium fluoride (251 mg, 4.32 mmol) in THF (1.0 mL) and methanol (1.0 mL). The resulting solution was stirred for 2 h at 50° C. and applied onto a reverse phase column, eluting with acetonitrile (20-100%) in water (0.1% formic acid). The desired fractions were collected and concentrated under reduced pressure to remove most of the acetonitrile then lyophilized to yield the title compound as a yellow solid (167 mg, 83%).

(ES, m/z): [M+H]⁺=466.9.

¹H-NMR (300 MHz, DMSO-d₆) δ 10.08 (s, 1H), 8.81 (s, 1H), 7.59 (s, 3H), 7.25 (d, J=48.8 Hz, 4H), 6.65 (s, 2H), 5.11 (s, 1H), 4.40 (s, 2H), 2.78 (s, 4H), 2.53 (s, 3H), 2.30 (s, 3H).

### Example 32

### 2-[(4-{[2-(Dimethylamino)ethyl](methyl)amino}-3-methylphenyl)amino]-8-phenyl-5-[2-(triisopropylsilyl)ethynyl]pyrido[2,3-d]pyrimidin-7-one

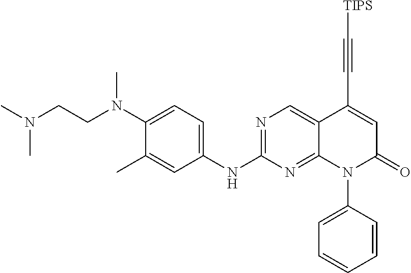

General procedure 13 was applied to 2-methanesulfonyl-8-phenyl-5-[2-(triisopropylsilyl)ethynyl]pyrido[2,3-d]pyrimidin-7-one (400 mg, 0.830 mmol) with trifluoroacetic acid (189 mg, 1.66 mmol) and N¹-[2-(dimethylamino)ethyl]-N^{1,2}-dimethylbenzene-1,4-diamine (206 mg, 0.996 mmol) in 2-butanol (4.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (250 mg, 49%).

(ES, m/z): [M+H]⁺=609.4.

### 2-[(4-{[2-(Dimethylamino)ethyl](methyl)amino}-3-methylphenyl)amino]-5-ethynyl-8-phenylpyrido[2,3-d]pyrimidin-7-one

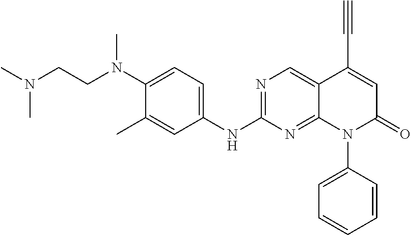 [SMILES: C#Cc1cc(=O)n(-c2ccccc2)c2nc(Nc3ccc(N(C)CCN(C)C)c(C)c3)ncc12]

General procedure 15 was applied to 2-[(4-{[2-(dimethylamino)ethyl](methyl)amino}-3-methylphenyl)amino]-8-phenyl-5-[2-(triisopropylsilyl)ethynyl]pyrido[2,3-d]pyrimidin-7-one (250 mg, 0.411 mmol) with potassium fluoride (238 mg, 4.11 mmol) in THF (3.0 mL), methanol (3.0 mL) and water (0.50 mL). The resulting solution was stirred for 2 h at 70° C., applied directly onto a reverse phase column, eluting with acetonitrile (20-100%) in water (0.1% formic acid). The desired fractions were collected and concentrated under reduced pressure to remove most of the acetonitrile then lyophilized to yield the title compound as an orange solid (43.5 mg, 23%).

(ES, m/z): [M+H]⁺=453.0.

¹H-NMR (300 MHz, DMSO-d₆) δ 10.12 (s, 1H), 8.82 (s, 1H), 7.60-7.52 (m, 3H), 7.35-7.33 (m, 2H), 7.08-7.03 (m, 2H), 6.67-6.64 (m, 2H), 5.10 (s, 1H), 2.83 (d, J=5.4 Hz, 2H), 2.53 (s, 3H), 2.32 (d, J=2.6 Hz, 2H), 2.15 (s, 6H), 1.97 (s, 3H).

### Example 33

### N,N-dimethyl-1-(4-nitrophenyl)piperidin-4-amine

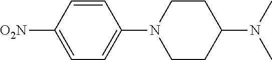 [SMILES: CN(C)C1CCN(c2ccc([N+](=O)[O-])cc2)CC1]

General procedure 11 was applied to 4-fluoronitrobenzene (3.50 g, 24.6 mmol), potassium carbonate (8.15 g, 58.9 mmol) and N,N-dimethylpiperidin-4-amine (3.78 g, 29.5 mmol) in DMF (35 mL). The residue was purified by flash column chromatography eluting with ethyl acetate (0-75%) in 40-60 petroleum ether to yield the title compound as a light yellow solid (4.50 g, 73%).

(ES, m/z): [M+H]⁺=250.2.

### 1-(4-Aminophenyl)-N,N-dimethylpiperidin-4-amine

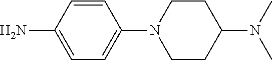 [SMILES: CN(C)C1CCN(c2ccc(N)cc2)CC1]

General procedure 12 was applied to N,N-dimethyl-1-(4-nitrophenyl)piperidin-4-amine (4.00 g, 15.9 mmol) with palladium on carbon 5 wt. % (680 mg, 3.89 mmol) in methanol (40 mL) to yield the title compound as a purple solid (3.00 g, 81%).

(ES, m/z): [M+H]⁺=220.2.

### 2-({4-[4-(Dimethylamino)piperidin-1-yl]phenyl}amino)-8-phenyl-5-[2-(triisopropylsilyl)ethynyl]pyrido[2,3-d]pyrimidin-7-one

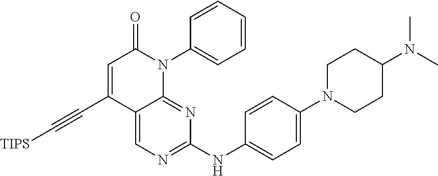

General procedure 13 was applied to 2-methanesulfonyl-8-phenyl-5-[2-(triisopropylsilyl)ethynyl]pyrido[2,3-d]pyrimidin-7-one (500 mg, 1.01 mmol) with 1-(4-aminophenyl)-N,N-dimethylpiperidin-4-amine (351 mg, 1.52 mmol) and trifluoroacetic acid (289 mg, 2.53 mmol) in 2-butanol (10 mL). The reaction mixture was stirred for 24 h at 110° C. and directly applied onto a reverse phase column, eluting with acetonitrile (10-100%) in water (0.1% trifluoroacetic acid) to yield the title compound as a yellow solid (450 mg, 67%).

(ES, m/z): [M+H]⁺=621.5.

### 2-({4-[4-(Dimethylamino)piperidin-1-yl]phenyl}amino)-5-ethynyl-8-phenylpyrido[2,3-d]pyrimidin-7-one

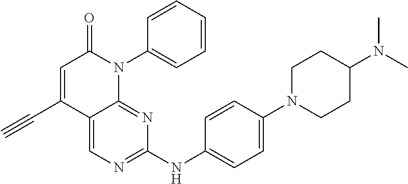 [SMILES: C#Cc1cc(=O)n(-c2ccccc2)c2nc(Nc3ccc(N4CCC(N(C)C)CC4)cc3)ncc12]

General procedure 15 was applied to 2-({4-[4-(dimethylamino)piperidin-1-yl]phenyl}amino)-8-phenyl-5-[2-(triisopropylsilyl)ethynyl]pyrido[2,3-d]pyrimidin-7-one (200 mg, 0.300 mmol) with potassium fluoride (174 mg, 3.00 mmol) in THF (10 mL), methanol (5.0 mL) and water (0.20 mL). The reaction mixture was stirred for 5 h at room temperature and directly applied onto a reverse phase column, eluting with acetonitrile (10-100%) in water (0.1% NH₄HCO₃). The desired fractions were collected and concentrated under reduced pressure to remove most of the acetonitrile then lyophilized to yield the title compound as a yellow solid (40.0 mg, 28%).

(ES, m/z): [M+H]⁺=465.2.

¹H-NMR (400 MHz, DMSO-d₆) δ 10.13 (s, 1H), 8.80 (s, 1H), 7.59 (p, J=6.0 Hz, 3H), 7.38-7.32 (m, 2H), 7.20-7.09 (m, 2H), 6.63 (d, J=11.3 Hz, 3H), 5.12 (s, 1H), 3.64 (d, J=12.0 Hz, 2H), 3.27 (ddt, J=12.2, 8.3, 4.1 Hz, 1H), 2.76 (d, J=4.8 Hz, 6H), 2.70-2.62 (m, 2H), 2.08 (d, J=11.9 Hz, 2H), 1.74 (dt, J=12.7, 6.3 Hz, 2H).

### Example 34

### N¹,N¹,N²-Trimethyl-N²-(4-nitrophenyl)ethane-1,2-diamine

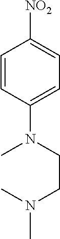 [SMILES: CN(C)CCN(C)c1ccc([N+](=O)[O-])cc1]

General procedure 11 was applied to 4-fluoro-1-nitrobenzene (3.10 mL, 29.2 mmol) with N¹,N¹,N³-trimethylethylenediamine (3.70 mL, 29.2 mmol) and potassium carbonate (6.66 g, 43.8 mmol) in DMSO (29 mL). The crude material was purified by flash column chromatography eluting with methanol (0-10%) in dichloromethane to yield the title compound as a yellow solid (6.35 g, 28.4 mmol, 97%).

(ES, m/z): [M+H]⁺=224.2.

### M-(2-(Dimethylamino)ethyl)-N-methylbenzene-1,4-diamine

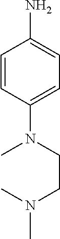

General procedure 12 was applied to N¹,N¹,N²-trimethyl-N²-(4-nitrophenyl)ethane-1,2-diamine (6.36 g, 28.5 mmol) with palladium on carbon 5 wt. % (303 mg, 0.285 mmol) in ethanol (142.0 mL). The crude material was purified by flash column chromatography eluting with methanol (0-20%) in dichloromethane to yield the title compound as a purple oil (4.19 g, 21.7 mmol, 76%).

### 2-((4-((2-(Dimethylamino)ethyl)(methyl)amino)phenyl)amino)-8-phenyl-5-((triisopropylsilyl)ethynyl)pyrido[2,3-d]pyrimidin-7(8H)-one

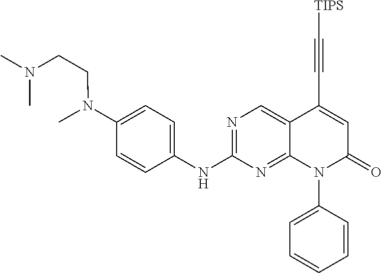

General procedure 13 was applied to 2-(methylsulfonyl)-8-phenyl-5-((triisopropylsilyl)ethynyl)pyrido[2,3-d]pyrimidin-7(8H)-one (974 mg, 2.02 mmol) with N¹-(2-(dimethylamino)ethyl)-N¹-methylbenzene-1,4-diamine (410 mg, 2.12 mmol), trifluoroacetic acid (162 μL, 2.12 mmol) and acetonitrile (20 mL). The crude material was purified by flash column chromatography eluting with methanol (0-10%) in dichloromethane to yield the title compound as a red solid (577.1 mg, 0.969 mmol, 48%).

(ES, m/z): [M+H]⁺=595.5.

### 2-((4-((2-(Dimethylamino)ethyl)(methyl)amino)phenyl)amino)-5-ethynyl-8-phenylpyrido[2,3-d]pyrimidin-7(8H)-one

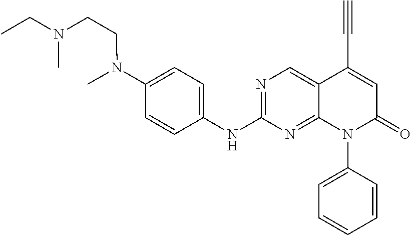 [SMILES: C#Cc1cc(=O)n(-c2ccccc2)c2nc(Nc3ccc(N(C)CCN(C)CC)cc3)ncc12]

General procedure 15 was applied to 2-((4-((2-(dimethylamino)ethyl)(methyl)amino)phenyl)amino)-8-phenyl-5-((triisopropylsilyl)ethynyl)pyrido[2,3-d]pyrimidin-7(8H)-one (577 mg, 0.973 mmol) with potassium fluoride (1.13 g, 19.4 mmol) in DMF (10 mL). The crude material was purified by flash column chromatography eluting with methanol (0-10%) in dichloromethane to yield the title compound as a red solid (302 mg, 0.689 mmol, 71%).

(ES, m/z): [M+H]⁺=439.3.

¹H NMR (500 MHz, DMSO-d₆) δ 9.97 (s, 1H), 8.76 (s, 1H), 7.59 (t, J=7.6 Hz, 2H), 7.52 (t, J=7.4 Hz, 1H), 7.35 (d, J=7.6 Hz, 2H), 7.07 (d, J=8.5 Hz, 2H), 6.61 (s, 1H), 6.28 (d, J=8.7 Hz, 2H), 5.12 (s, 1H), 3.32-3.23 (m, 2H), 2.80 (s, 3H), 2.30 (t, J=7.3 Hz, 2H), 2.20 (s, 6H).

### Example 35

### 1-(3-Methoxy-4-nitrophenyl)-4-methylpiperazine

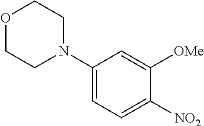 [SMILES: COc1cc(N2CCOCC2)ccc1[N+](=O)[O-]]

General procedure 11 was applied to 4-fluoro-2-methoxy-1-nitrobenzene (1.50 g, 8.76 mmol) with morpholine (0.75 mL, 8.76 mmol) and potassium carbonate (2.00 g, 13.1 mmol) in DMSO (18 mL) to yield the title compound as a yellow solid (1.62 g, 6.79 mmol, 78%) which was used without further purification.

(ES, m/z): [M+H]⁺=239.1.

### 2-Methoxy-4-morpholinoaniline

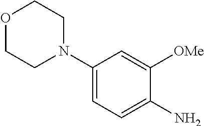 [SMILES: COc1cc(N2CCOCC2)ccc1N]

General procedure 12 was applied to 1-(3-methoxy-4-nitrophenyl)-4-methylpiperazine (1.00 g, 4.20 mmol) with palladium on carbon 5 wt. % (223 mg, 0.200 mmol) in ethanol (21.0 mL). The crude material was purified by flash column chromatography eluting with methanol (0-20%) in dichloromethane to yield the title compound as a purple oil (588 mg, 2.82 mmol, 67%).

### 2-(Methylthio)-8-phenyl-5-((trimethylylsilyl)ethynyl)pyrido[2,3-d]pyrimidin-7(8H)-one

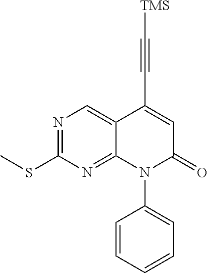 [SMILES: CSc1ncc2c(C#C[Si](C)(C)C)cc(=O)n(-c3ccccc3)c2n1]

General procedure 9 was applied to 2-(methylthio)-7-oxo-8-phenyl-7,8-dihydropyrido[2,3-d]pyrimidin-5-yl trifluoromethanesulfonate (1.05 g, 2.51 mmol) with trimethylsilylacetylene (70.0 μL, 5.00 mmol), copper(I) iodide (48.0 mg, 0.251 mmol), bis(triphenylphsophine)palladium(II) dichloride (176 mg, 0.251 mmol) in DMF (12.5 mL) and N,N-diisopropylethylamine (101.0 mL). The crude material was purified by flash column chromatography eluting with ethyl acetate (25-100%) in 40-60 petroleum ether to yield the title compound as a light yellow solid (798 mg, 87%).

(ES, m/z): [M+H]⁺=366.2

### 2-(Methylsulfonyl)-8-phenyl-5-((trimethylsilyl)ethynyl)pyrido[2,3-d]pyrimidin-7(8H)-one

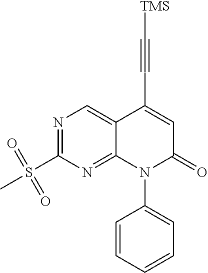 [SMILES: C[Si](C)(C)C#Cc1cc(=O)n(-c2ccccc2)c2nc(S(C)(=O)=O)ncc12]

General procedure 10 was applied to 2-(methylthio)-8-phenyl-5-((trimethylsilyl)ethynyl)pyrido[2,3-d]pyrimidin-7(8H)-one (798 mg, 2.18 mmol) with m-CPBA (1.47 mg, 6.54 mmol) in dichloromethane (22.0 mL) to yield the title compound as a yellow solid (1.05 g, 100%).

(ES, m/z): [M+H]⁺=398.2

### 2-((2-Methoxy-4-morpholinophenyl)amino)-8-phenyl-5-((trimethylsilyl)ethynyl)pyrido[2,3-d]pyrimidin-7(8H)-one

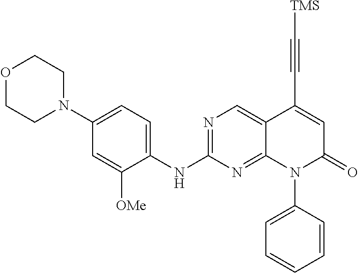 [SMILES: COc1cc(N2CCOCC2)ccc1Nc1ncc2c(C#C[Si](C)(C)C)cc(=O)n(-c3ccccc3)c2n1]

General procedure 13 was applied to 2-(methylsulfonyl)-8-phenyl-5-((trimethylsilyl)ethynyl)pyrido[2,3-d]pyrimidin-7(8H)-one (400 mg, 1.01 mmol) with 2-methoxy-4-morpholinoaniline (336 mg, 1.52 mmol), trifluoroacetic acid (120 μL, 1.52 mmol) and acetonitrile (10 mL). The crude material was purified by flash column chromatography eluting with methanol (0-10%) in dichloromethane to yield the title compound as a red solid (184 mg, 0.350 mmol, 35%).

(ES, m/z): [M+H]⁺=526.4.

### 5-Ethynyl-2-((2-methoxy-4-morpholinophenyl)amino)-8-phenylpyrido[2,3-d]pyrimidin-7(8H)-one

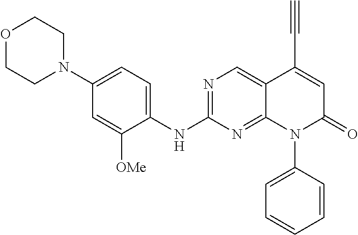 [SMILES: C#Cc1cc(=O)n(-c2ccccc2)c2nc(Nc3ccc(N4CCOCC4)cc3OC)ncc12]

A solution of 2-((2-methoxy-4-morpholinophenyl)amino)-8-phenyl-5-((trimethylsilyl)ethynyl)pyrido[2,3-d]pyrimidin-7(8H)-one (77.0 mg, 0.146 mmol) and potassium carbonate (20.0 mg, 0.146 mmol) in methanol (1.5 mL) was stirred at room temperature for 30 minutes. The reaction mixture was quenched with water (30 mL), and the resulting solids were collected by vacuum filtration and dried under vacuum at 40° C. overnight to yield the title compound as a red solid (54.5 mg, 0.120 mmol, 82%).

(ES, m/z): [M+H]⁺=454.3

¹H NMR (500 MHz, CDCl₃) δ 8.78 (s, 1H), 7.86 (s, 1H), 7.58-7.45 (m, 3H), 7.27-7.21 (m, 2H), 6.65 (s, 1H), 6.35 (d, J=2.5 Hz, 1H), 5.90 (s, 1H), 3.82-3.78 (m, 4H), 3.77 (s, 3H), 3.60 (s, 1H), 2.99 (t, J=4.7 Hz, 4H), 1.53 (s, 2H).

### Example 36

### 3-Methoxy-N-(3-methoxy-4-nitrophenyl)-N-methylpropanamide

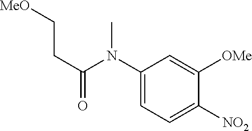 [SMILES: COCCC(=O)N(C)c1ccc([N+](=O)[O-])c(OC)c1]

A solution of 4-fluoro-2-methoxy-1-nitrobenzene (470 mg, 2.58 mmol) with 3-methoxypropionyl chloride (0.39 mL, 3.57 mmol) and pyridine (0.44 mL, 5.48 mmol) in acetonitrile (5.5 mL) was stirred at room temperature overnight. The crude material was concentrated under reduced pressure, the residue was dissolved in ethyl acetate (20 mL) and washed with saturated aqueous copper(II) sulphate solution (10 mL). The aqueous layer was extracted with ethyl acetate (2×20 mL). The combined organic layers were washed with water (3×10 mL), dried (MgSO₄) and concentrated under reduced pressure to yield the title compound as a yellow solid (620 mg, 2.31 mmol, 90%) which was used without further purification.

(ES, m/z): [M+H]⁺=269.2.

### N-(4-Amino-3-methoxyphenyl)-3-methoxy-N-methylpropanamide

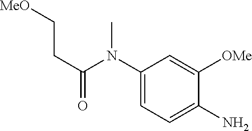 [SMILES: COCCC(=O)N(C)c1ccc(N)c(OC)c1]

General procedure 12 was applied to 3-methoxy-N-(3-methoxy-4-nitrophenyl)-N-methylpropanamide (620 mg, 2.31 mmol) with palladium on carbon 5 wt. % (246 mg, 0.231 mmol) in acetic acid (1.0 mL) and ethanol (9.0 mL). The crude material was purified by flash column chromatography eluting with methanol (0-20%) in dichloromethane to yield the title compound as a purple oil (470 mg, 1.97 mmol, 85%).

(ES, m/z): [M+H]⁺=239.2.

### 3-Methoxy-N-(3-methoxy-4-((7-oxo-8-phenyl-5-((trimethylsilyl)ethynyl)-7,8-dihydropyrido[2,3-d]pyrimidin-2-yl)amino)phenyl)-N-methylpropanamide

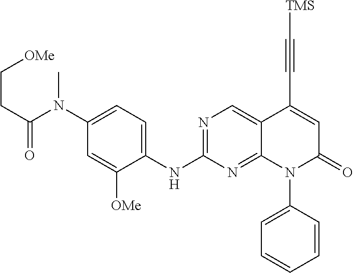 [SMILES: COCCC(=O)N(C)c1ccc(Nc2ncc3c(C#C[Si](C)(C)C)cc(=O)n(-c4ccccc4)c3n2)c(OC)c1]

General procedure 13 was applied to 2-(methylsulfonyl)-8-phenyl-5-((trimethylsilyl)ethynyl)pyrido[2,3-d]pyrimidin-7(8H)-one (400 mg, 1.01 mmol) with N-(4-amino-3-methoxyphenyl)-3-methoxy-N-methylpropanamide (362 mg, 1.52 mmol), trifluoroacetic acid (120 μL, 1.52 mmol) and acetonitrile (10 mL). The crude material was purified by flash column chromatography eluting with methanol (0-10%) in dichloromethane to yield the title compound as a red solid (325 mg, 0.586 mmol, 58%).

(ES, m/z): [M+H]⁺=556.5.

### N-(4-((5-Ethynyl-7-oxo-8-phenyl-7,8-dihydropyrido[2,3-d]pyrimidin-2-yl)amino)-3-methoxyphenyl)-3-methoxy-N-methylpropanamide

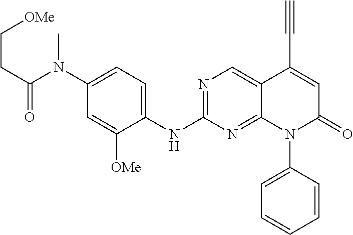 [SMILES: C#Cc1cc(=O)n(-c2ccccc2)c2nc(Nc3ccc(N(C)C(=O)CCOC)cc3OC)ncc12]

A solution of 2-((2-methoxy-4-morpholinophenyl)amino)-8-phenyl-5-((trimethylsilyl)ethynyl)pyrido[2,3-d]pyrimidin-7(8H)-one (325 mg, 0.586 mmol) and potassium carbonate (81.0 mg, 0.586 mmol) in methanol (6.0 mL) was stirred at room temperature for 1 h. The reaction mixture was quenched with water (30 mL), and the resulting solids collected by vacuum filtration. The crude material was purified by reverse phase flash column chromatography eluting with acetonitrile (5-95%) in water (0.1% formic acid) to yield the title compound as a red solid (82.9 mg, 0.171 mmol, 29%).

(ES, m/z): [M+H]⁺=484.4.

¹H NMR (500 MHz, CDCl₃) δ 8.89 (s, 1H), 8.04 (s, 1H), 7.70-7.54 (m, 3H), 7.48 (s, 2H), 7.30 (d, J=7.4 Hz, 2H), 6.76 (s, 1H), 6.65 (s, 1H), 6.29 (s, 1H), 3.85 (s, 3H), 3.76 (s, 1H), 3.62 (t, J=6.5 Hz, 2H), 3.31 (s, 3H), 3.22 (s, 3H), 2.39-2.11 (m, 2H).

### Example 37

### 1-Methyl-4-(4-nitrophenyl)piperazine

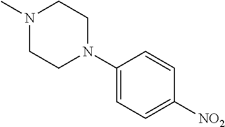 [SMILES: CN1CCN(c2ccc([N+](=O)[O-])cc2)CC1]

General procedure 11 was applied to 4-fluoro-1-nitrobenzene (1.55 mL, 14.6 mmol) with 1-methylpiperazine (1.62.0 mL, 14.6 mmol) and potassium carbonate (3.33 g, 21.9 mmol) in DMSO (15 mL) to yield the title compound as a yellow solid (2.42 g, 10.9 mmol, 75%) which was used without further purification.

(ES, m/z): [M+H]⁺=222.2.

### 4-(4-Methylpiperazin-1-yl)aniline

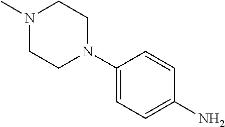

General procedure 12 was applied to 1-methyl-4-(4-nitrophenyl)piperazine (1.00 g, 4.52 mmol) with palladium on carbon 5 wt. % (45.0 mg, 0.045 mmol) in ethanol (45 mL). The crude material was purified by flash column chromatography eluting with methanol (0-20%) in dichloromethane to yield the title compound as a purple oil (756 mg, 3.95 mmol, 87%).

### 2-((4-(4-Methylpiperazin-1-yl)phenyl)amino)-8-phenyl-5-((trimethylsilyl)ethynyl)pyrido[2,3-d]pyrimidin-7(8H)-one

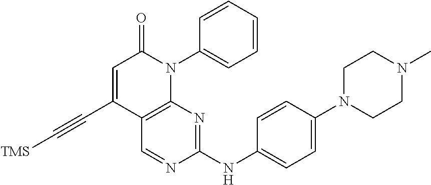 [SMILES: CN1CCN(c2ccc(Nc3ncc4c(C#C[Si](C)(C)C)cc(=O)n(-c5ccccc5)c4n3)cc2)CC1]

General procedure 13 was applied to 2-(methylsulfonyl)-8-phenyl-5-((trimethylsilyl)ethynyl)pyrido[2,3-d]pyrimidin-7(8H)-one (180 mg, 0.451 mmol) with 4-(4-methylpiperazin-1-yl)aniline (130 mg, 0.681 mmol), trifluoroacetic acid (50.0 μL, 0.681 mmol) and acetonitrile (4.5 mL). The crude material was purified by flash column chromatography eluting with methanol (0-10%) in dichloromethane to yield the title compound as a red solid (136 mg, 0.267 mmol, 59%).

(ES, m/z): [M+H]⁺=509.4.

### 5-Ethynyl-2-((4-(4-methylpiperazin-1-yl)phenyl)amino)-8-phenylpyrido[2,3-d]pyrimidin-7(8H)-one

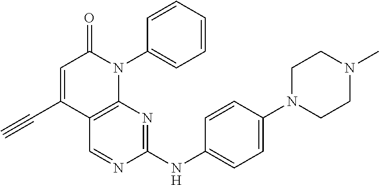 [SMILES: C#Cc1cc(=O)n(-c2ccccc2)c2nc(Nc3ccc(N4CCN(C)CC4)cc3)ncc12]

A solution of 2-((4-(4-methylpiperazin-1-yl)phenyl)amino)-8-phenyl-5-((trimethylsilyl)ethynyl)pyrido[2,3-d]pyrimidin-7(8H)-one (135 mg, 0.265 mmol) and potassium carbonate (37.0 mg, 0.265 mmol) in methanol (2.6 mL) was stirred at room temperature for 30 minutes. The reaction mixture was quenched with water (30 mL), and the resulting solids were collected by vacuum filtration and dried under vacuum at 40° C. overnight. The crude material was triturated with diethyl ether and ethyl acetate to yield the title compound as an orange solid (41.9 mg, 96.1 μmol, 36%).

(ES, m/z): [M+H]⁺=437.3

¹H NMR (500 MHz, CDCl₃) δ 8.70 (s, 1H), 7.43 (dt, J=16.3, 7.5 Hz, 3H), 7.13 (d, J=7.7 Hz, 2H), 6.88 (d, J=14.5 Hz, 2H), 6.58 (s, 1H), 6.45 (s, 2H), 3.52 (s, 1H), 2.95 (s, 4H), 2.43 (t, J=5.0 Hz, 4H), 2.21 (s, 3H).

### Example 38

### tert-Butyl 7-(4-nitrophenyl)-4,7-diazaspiro[2.5]octane-4-carboxylate

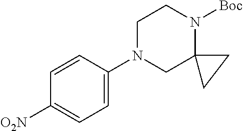 [SMILES: CC(C)(C)OC(=O)N1CCN(c2ccc([N+](=O)[O-])cc2)CC12CC2]

General procedure 11 was applied to 4-fluoronitrobenzene (1.00 g, 6.80 mmol), tert-butyl 4,7-diazaspiro[2.5]octane-4-carboxylate (1.73 g, 8.16 mmol) and potassium carbonate (2.26 g, 16.3 mmol) in DMF (10 mL). The crude residue was purified by flash column chromatography, eluting with ethyl acetate (8%) in 40-60 petroleum ether to afford the title compound as a yellow solid (600 mg, 25%).

(ES, m/z): [M+H]⁺=334.2.

### tert-Butyl 7-(4-aminophenyl)-4,7-diazaspiro[2.5]octane-4-carboxylate

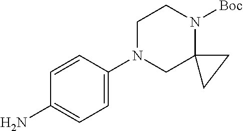 [SMILES: CC(C)(C)OC(=O)N1CCN(c2ccc(N)cc2)CC12CC2]

General procedure 12 was applied to tert-butyl 7-(4-nitrophenyl)-4,7-diazaspiro[2.5]octane-4-carboxylate (600 mg, 1.80 mmol) and palladium on carbon 5 wt. % (120 mg, 1.13 mmol) in methanol (6.0 mL) to yield the title compound as a brown solid (450 mg, 82%).

(ES, m/z): [M+H]⁺=304.2.

### tert-Butyl 7-[4-({7-oxo-8-phenyl-5-[2-(triisopropylsilyl)ethynyl]pyrido[2,3-d]pyrimidin-2-yl}amino)phenyl]-4,7-diazaspiro[2.5]octane-4-carboxylate

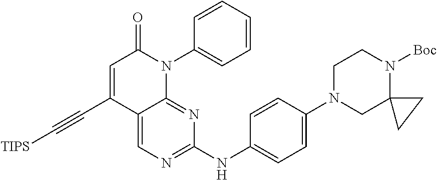

General procedure 13 was applied to 2-methanesulfonyl-8-phenyl-5-[2-(triisopropylsilyl)ethynyl]pyrido[2,3-d]pyrimidin-7-one (400 mg, 0.830 mmol), trifluoroacetic acid (189 mg, 1.66 mmol) and tert-butyl 7-(4-aminophenyl)-4,7-diazaspiro[2.5]octane-4-carboxylate (302 mg, 0.996 mmol) in 2-butanol (4.0 mL). The solution mixture was stirred for 1 h at 100° C. The crude residue was purified by reverse phase flash column chromatography eluting with acetonitrile (30-60%) in water (0.1% formic acid) to yield the title compound as a yellow solid (350 mg, 60%).

(ES, m/z): [M+H]⁺=705.4.

### 2-[(4-{4,7-Diazaspiro[2.5]octan-7-yl}phenyl)amino]-8-phenyl-5-[2-(triisopropylsilyl)ethynyl]pyrido[2,3-d]pyrimidin-7-one

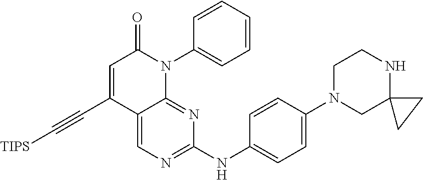

To a solution of tert-butyl 7-[4-({7-oxo-8-phenyl-5-[2-(triisopropylsilyl)ethynyl]pyrido[2,3-d]pyrimidin-2-yl}amino)phenyl]-4,7-diazaspiro[2.5]octane-4-carboxylate (350 mg, 0.496 mmol, 1.0 eq.) in methanol (3.5 mL) was added hydrochloric acid in methanol (0.35 mL) and the resulting solution was stirred for 4 h at room temperature. The resulting mixture was concentrated under reduced pressure to yield the title compound as a yellow solid (250 mg, 83%).

(ES, m/z): [M+H]⁺=605.3.

### 2-[(4-{4,7-Diazaspiro[2.5]octan-7-yl}phenyl)amino]-5-ethynyl-8-phenylpyrido[2,3-d]pyrimidin-7-one

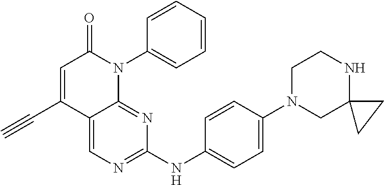 [SMILES: C#Cc1cc(=O)n(-c2ccccc2)c2nc(Nc3ccc(N4CCNC5(CC5)C4)cc3)ncc12]

General procedure 15 was applied to 2-[(4-{4,7-diazaspiro[2.5]octan-7-yl}phenyl)amino]-8-phenyl-5-[2-(triisopropylsilyl)ethynyl]pyrido[2,3-d]pyrimidin-7-one (250 mg, 0.413 mmol) and potassium fluoride (480 mg, 8.26 mmol) in THF (1.5 mL) and methanol (3.0 mL). The resulting mixture was stirred for 2 h at 80° C. The crude residue was purified by reverse phase flash column chromatography eluting with acetonitrile (30-60%) in water (0.1% formic acid) to yield the title compound as a yellow solid (72.0 mg, 36%).

(ES, m/z): [M+H]⁺=449.2.

¹H NMR (400 MHz, DMSO-d₆) δ 10.00 (s, 1H), 8.78 (s, 1H), 7.59 (d, J=7.7 Hz, 3H), 7.39-7.28 (m, 2H), 7.10 (s, 2H), 6.62 (s, 1H), 6.48 (s, 2H), 5.10 (s, 1H), 2.92 (dt, J=9.8, 5.1 Hz, 4H), 2.80 (s, 2H), 2.54 (s, 1H), 0.53 (s, 4H).

### Example 39

### tert-Butyl 4-[3-(methoxymethyl)azetidin-1-yl]piperidine-1-carboxylate

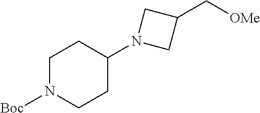 [SMILES: COCC1CN(C2CCN(C(=O)OC(C)(C)C)CC2)C1]

A solution of 3-(methoxymethyl)azetidine (2.00 g, 19.8 mmol, 1.0 eq.), tert-butyl 4-oxopiperidine-1-carboxylate (7.88 g, 39.5 mmol, 2.0 eq.), sodium cyanoborohydride (2.49 g, 39.5 mmol, 2.0 eq.), acetic acid (1.00 mL, 17.5 mmol, 0.88 eq.) in methanol (10 mL) and dichloromethane (10 mL) was stirred at room temperature until completion. The precipitates were removed by filtration and the filtrate was concentrated under reduced pressure to yield the title compound as a white semi-solid (1.50 g, 27%).

(ES, m/z): [M+H]⁺=285.

### 4-[3-(Methoxymethyl)azetidin-1-yl]piperidine

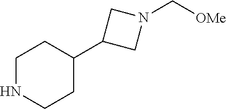 [SMILES: COCN1CC(C2CCNCC2)C1]

To a stirred solution of tert-butyl 4-[3-(methoxymethyl) azetidin-1-yl]piperidine-1-carboxylate (1.50 g, 5.27 mmol, 1.0 eq.) was added 4M hydrogen chloride in 1,4-dioxane (15 mL) dropwise at room temperature. The resulting mixture was concentrated under reduced pressure to yield the title compound as a white semi-solid (650 mg, 61%).

(ES, m/z): [M+H]⁺=185.

### 4-[3-(Methoxymethyl)azetidin-1-yl]-1-(4-nitrophenyl)piperidine

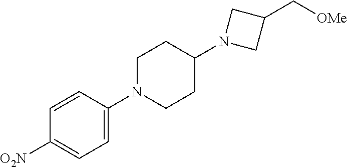 [SMILES: COCC1CN(C2CCN(c3ccc([N+](=O)[O-])cc3)CC2)C1]

General procedure 11 was applied to 4-[3-(methoxymethyl)azetidin-1-yl]piperidine (1.50 g, 8.14 mmol), 4-fluoronitrobenzene (1.38 g, 9.77 mmol) and potassium carbonate (2.70 g, 19.5 mmol) in DMF (15 mL). The crude material was purified by flash column chromatography eluting with ethyl acetate (0-75%) in 40-60 petroleum ether to yield the title compound as a light yellow solid (650 mg, 26%).

(ES, m/z): [M+H]⁺=306.1.

### 4-{4-[3-(Methoxymethyl)azetidin-1-yl]piperidin-1-yl}aniline

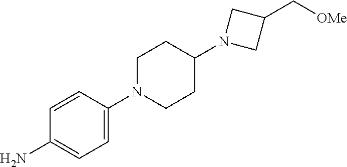 [SMILES: COCC1CN(C2CCN(c3ccc(N)cc3)CC2)C1]

General procedure 12 was applied to 4-[3-(methoxymethyl)azetidin-1-yl]-1-(4-nitrophenyl)piperidine (650 mg, 2.13 mmol) with palladium on carbon 5 wt. % (6.50 mg) in methanol (6.5 mL) to yield the title compound as a purple solid (500 mg, 83%).

(ES, m/z): [M+H]⁺=276.1.

### 2-[(4-{4-[3-(Methoxymethyl)azetidin-1-yl]piperidin-1-yl}phenyl) amino]-8-phenyl-5-[2-(triisopropylsilyl)ethynyl]pyrido[2,3-d]pyrimidin-7-one

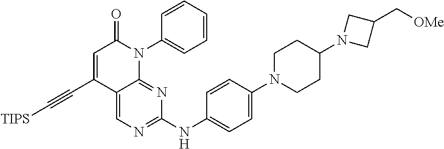

General procedure 13 was applied to 2-methanesulfonyl-8-phenyl-5-[2-(triisopropylsilyl)ethynyl]pyrido[2,3-d]pyrimidin-7-one (500 mg, 1.04 mmol) with 4-{4-[3-(methoxymethyl) azetidin-1-yl]piperidin-1-yl}aniline (343 mg, 1.26 mmol) and trifluoroacetic acid (284 mg, 2.49 mmol) in 2-butanol (10 mL). The reaction mixture was stirred at 110° C. for 24 h. The crude material was purified by reverse phase flash column chromatography eluting with acetonitrile (10-100%) in water (0.1% trifluoroacetic acid) to yield the title compound as a red solid (290 mg, 40%).

(ES, m/z): [M+H]⁺=677.

### 5-Ethynyl-2-[(4-{4-[3-(methoxymethyl) azetidin-1-yl]piperidin-1-yl}phenyl)amino]-8-phenylpyrido[2,3-d]pyrimidin-7-one

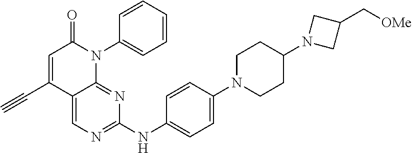 [SMILES: C#Cc1cc(=O)n(-c2ccccc2)c2nc(Nc3ccc(N4CCC(N5CC(COC)C5)CC4)cc3)ncc12]

General procedure 15 was applied to 2-[(4-{4-[3-(methoxymethyl)azetidin-1-yl]piperidin-1-yl}phenyl)amino]-8-phenyl-5-[2-(triisopropylsilyl)ethynyl]pyrido[2,3-d]pyrimidin-7-one (350 mg, 0.517 mmol) with potassium fluoride (300 mg, 5.17 mmol) in methanol (2.00 mL), THF (4.00 mL) and water (0.10 mL). The reaction mixture was stirred at room temperature for 2 h. The reaction mixture was purified by reverse phase flash column chromatography eluting with acetonitrile (10-100%) in water (0.1% formic acid) to yield the title compound as a yellow solid (18.0 mg, 6%).

(ES, m/z): [M+H]⁺=521.3.

¹H NMR (400 MHz, DMSO-d₆) δ 10.01 (s, 1H), 8.78 (s, 1H), 7.59 (d, J=7.7 Hz, 3H), 7.40-7.29 (m, 2H), 7.09 (d, J=8.3 Hz, 2H), 6.62 (s, 1H), 6.51 (s, 2H), 5.10 (s, 1H), 3.42 (d, J=6.7 Hz, 2H), 3.33 (s, 3H), 3.24 (s, 3H), 2.89 (t, J=6.7 Hz, 2H), 2.60 (dd, J=14.6, 8.5 Hz, 4H), 2.16 (s, 1H), 1.75-1.63 (m, 2H), 1.25-1.19 (m, 2H).

### Example 40

### tert-Butyl 4-[3-(2-hydroxypropan-2-yl)azetidin-1-yl]piperidine-1-carboxylate

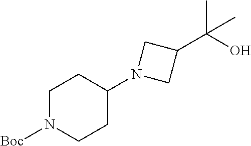 [SMILES: CC(C)(C)OC(=O)N1CCC(N2CC(C(C)(C)O)C2)CC1]

A solution of 2-(azetidin-3-yl)propan-2-ol (3.00 g, 26.0 mmol, 1.0 eq.) and tert-butyl 4-oxopiperidine-1-carboxylate (10.4 g, 52.1 mmol, 2.0 eq.) in dichloromethane (15 mL), methanol (15 mL) and acetic acid (1.5 mL) was stirred for 30 minutes at room temperature. Sodium cyanoborohydride (3.27 g, 52.1 mmol, 2.0 eq.) was added and the resulting solution was stirred overnight at room temperature. The resulting solution was applied onto a reverse phase column, eluting with acetonitrile (90-100%) in water (0.1% formic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a white solid (1.80 g, 23%).

(ES, m/z): [M+H]⁺=299.

### 2-[1-(Piperidin-4-yl)azetidin-3-yl]propan-2-ol

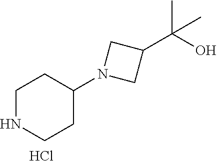

A solution of tert-butyl 4-[3-(2-hydroxypropan-2-yl)azetidin-1-yl]piperidine-1-carboxylate (1.80 g, 6.03 mmol, 1.0 eq.) in hydrogen chloride in methanol (20 mL) was stirred for 3 h at 30° C. The reaction was concentrated under reduced pressure to yield the title compound as a white solid (1.10 g, 92%).

(ES, m/z): [M+H]⁺=199.

### 2-{1-[1-(4-Nitrophenyl)piperidin-4-yl]azetidin-3-yl}propan-2-ol

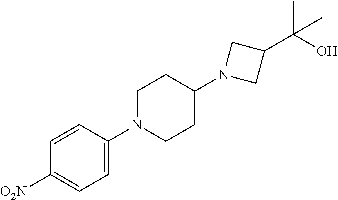 [SMILES: CC(C)(O)C1CN(C2CCN(c3ccc([N+](=O)[O-])cc3)CC2)C1]

General procedure 11 was applied to 2-[1-(piperidin-4-yl)azetidin-3-yl]propan-2-ol (1.40 g, 7.06 mmol) with 4-fluoronitrobenzene (1.20 g, 8.47 mmol) and potassium carbonate (1.95 g, 14.1 mmol) in DMF (15.0 mL). The crude material was purified by flash column chromatography eluting with ethyl acetate (20%) in 40-60 petroleum ether to yield the title compound as an orange solid (1.00 g, 44%).

(ES, m/z): [M+H]⁺=320.

### 2-{1-[1-(4-Aminophenyl)piperidin-4-yl]azetidin-3-yl}propan-2-ol

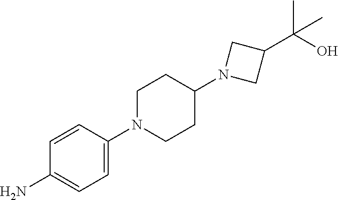 [SMILES: CC(C)(O)C1CN(C2CCN(c3ccc(N)cc3)CC2)C1]

General procedure 12 was applied to 2-{1-[1-(4-nitrophenyl)piperidin-4-yl]azetidin-3-yl}propan-2-ol (1.00 g, 3.13 mmol), with palladium on carbon 5 wt. % (200 mg) in methanol (10 mL) to yield the title compound as a grey solid (800 mg, 88%).

(ES, m/z): [M+H]⁺=290.

### 2-[(4-{4-[3-(2-Hydroxypropan-2-yl)azetidin-1-yl]piperidin-1-yl}phenyl)amino]-8-phenyl-5-[2-(triisopropylsilyl)ethynyl]pyrido[2,3-d]pyrimidin-7-one

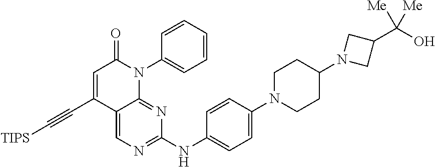

General procedure 13 was applied to 2-methanesulfonyl-8-phenyl-5-[2-(triisopropylsilyl)ethynyl]pyrido[2,3-d]pyrimidin-7-one (1.00 g, 2.08 mmol) with trifluoroacetic acid (473 mg, 4.15 mmol) and 2-{1-[1-(4-aminophenyl)piperidin-4-yl]azetidin-3-yl}propan-2-ol (721 mg, 2.49 mmol) in 2-butanol (10 mL). The resulting solution was stirred for 3 h at 100° C., diluted with methanol (10 mL) and applied onto a reverse phase column directly, eluting with acetonitrile (70-100%) in water (0.1% trifluoroacetic acid). The mixture was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a red solid (400 mg, 28%).

(ES, m/z): [M+H]⁺=691.

### 5-Ethynyl-2-[(4-{4-[3-(2-hydroxypropan-2-yl)azetidin-1-yl]piperidin-1-yl}phenyl)amino]-8-phenylpyrido[2,3-d]pyrimidin-7-one

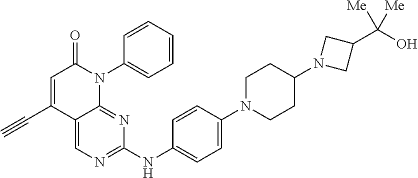 [SMILES: C#Cc1cc(=O)n(-c2ccccc2)c2nc(Nc3ccc(N4CCC(N5CC(C(C)(C)O)C5)CC4)cc3)ncc12]

General procedure 15 was applied to 2-[(4-{4-[3-(2-hydroxypropan-2-yl)azetidin-1-yl]piperidin-1-yl}phenyl)amino]-8-phenyl-5-[2-(triisopropylsilyl)ethynyl]pyrido[2,3-d]pyrimidin-7-one (200 mg, 0.289 mmol) with potassium fluoride (168 mg, 2.89 mmol) in THF (2.0 mL), DMF (2.0 mL) and water (0.50 mL). The resulting solution was stirred for 2 h at 70° C. The reaction mixture was directly applied onto a reverse phase column, eluting with acetonitrile (20-100%) in water (0.1% formic acid). The desired fractions were collected and concentrated under reduced pressure to remove most of the acetonitrile then lyophilized to yield the title compound as an orange solid (11.2 mg, 7%).

(ES, m/z): [M+H]⁺=535.

¹H-NMR (300 MHz, DMSO-d₆): δ 9.99 (s, 1H), 8.78 (s, 1H), 7.60-7.58 (m, 3H), 7.35 (d, J=3.9 Hz, 2H), 7.18-7.05 (m, 2H), 6.63 (s, 1H), 6.62-6.54 (m, 2H), 5.09 (s, 1H), 3.52-3.39 (m, 4H), 3.23-3.20 (m, 2H), 2.73-2.65 (m, 2H), 2.42-2.28 (m, 2H), 1.79-1.72 (m, 2H), 1.35-1.20 (m, 3H), 1.01 (s, 6H).

### Example 41

### 2-{[2-(Hydroxymethyl)phenyl]amino}-8-phenyl-5-[2-(triisopropylsilyl)ethynyl]pyrido[2,3-d]pyrimidin-7-one

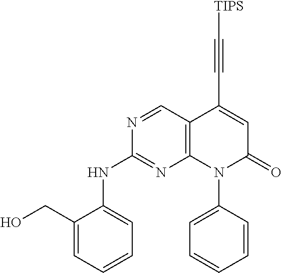

General procedure 13 was applied to 2-methanesulfonyl-8-phenyl-5-[2-(triisopropylsilyl)ethynyl]pyrido[2,3-d]pyrimidin-7-one (300 mg, 0.623 mmol) with pivalic acid (128 mg, 1.25 mmol) and isatoic anhydride (90.0 mg, 0.731 mmol) in 2-butanol (3.0 mL). The resulting mixture was stirred overnight at room temperature. The residue was purified by reverse phase flash column chromatography eluting with acetonitrile (10-100%) in water to yield the title compound as a light yellow solid (150 mg, 46%).

### 5-Ethynyl-2-{[2-(hydroxymethyl)phenyl]amino}-8-phenylpyrido[2,3-d]pyrimidin-7-one

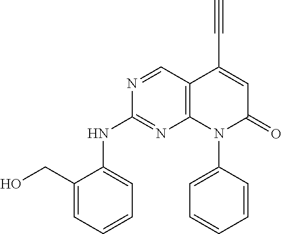 [SMILES: C#Cc1cc(=O)n(-c2ccccc2)c2nc(Nc3ccccc3CO)ncc12]

General procedure 15 was applied to 2-{[2-(hydroxymethyl)phenyl]amino}-8-phenyl-5-[2-(triisopropylsilyl) ethynyl]pyrido[2,3-d]pyrimidin-7-one (200 mg, 0.381 mmol) with potassium fluoride (330 mg, 5.68 mmol) in THF (2.0 mL) and water (2.0 mL). The resulting mixture was stirred overnight at 50° C. and extracted with dichloromethane (2×10 mL). The combined organic layers were dried (Na₂SO₄) and concentrated under reduced pressure. The crude material was purified by reverse phase flash column chromatography eluting with acetonitrile (10-60%) in water to yield the title compound as a yellow solid (49.3 mg, 35%).

(ES, m/z): [M+H]⁺=369.

¹H NMR (400 MHz, DMSO-d₆) δ 9.40 (s, 1H), 8.82 (s, 1H), 7.59-7.48 (m, 3H), 7.42 (dd, J=8.2, 1.2 Hz, 1H), 7.36-7.30 (m, 2H), 7.17 (d, J=7.5 Hz, 1H), 6.91 (t, J=7.5 Hz, 1H), 6.82 (s, 1H), 6.69 (s, 1H), 5.67 (s, 1H), 5.12 (s, 1H), 4.53 (s, 2H).

### Example 42

### Ethyl 4-(methylamino)-2-(methylsulfanyl)pyrimidine-5-carboxylate

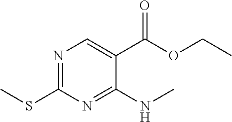

General procedure 1 was applied to ethyl 4-chloro-2-(methylsulfanyl)pyrimidine-5-carboxylate (80.0 g, 344 mmol) and methylamine (96.7 mL, 193 mmol) in THF (300 mL). The filtrate was concentrated under reduced pressure to afford the title compound as a white solid (20.5 g, 69%).

(ES, m/z): [M+H]⁺=228.1.

### 8-Methyl-2-(methylsulfanyl)-6H-pyrido[2,3-d]pyrimidine-5,7-dione

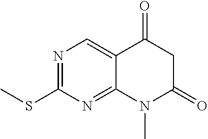

General procedure 7 was applied to ethyl 4-(methylamino)-2-(methylsulfanyl)pyrimidine-5-carboxylate (9.80 g, 43.1 mmol), with sodium hydride (5.17 g, 215 mmol), acetyl chloride (3.72 g, 47.4 mmol) and DMF (100 mL). The residue was purified by flash column chromatography eluting with methanol (5%) in dichloromethane to yield the title compound as a yellow solid (3.60 g, 36%).

(ES, m/z): [M+H]⁺=224.0.

### 8-Methyl-2-(methylsulfanyl)-7-oxopyrido[2,3-d]pyrimidin-5-yl trifluoromethanesulfonate

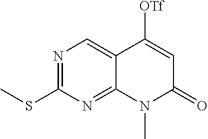 [SMILES: CSc1ncc2c(OS(=O)(=O)C(F)(F)F)cc(=O)n(C)c2n1]

General procedure 8 was applied to 8-methyl-2-(methylsulfanyl)-6H-pyrido[2,3-d]pyrimidine-5,7-dione (3.14 g, 14.1 mmol) with triethylamine (2.85 g, 28.2 mmol) and triflic anhydride (5.95 g, 21.1 mmol) in dichloromethane (30 mL). The residue was purified by flash column chromatography eluting with methanol (5%) in dichloromethane to afford the title compound as a light yellow solid (1.50 g, 27%).

(ES, m/z): [M+H]⁺=356.0.

### 8-Methyl-2-(methylsulfanyl)-5-[2-(triisopropylsilyl)ethynyl]pyrido[2,3-d]pyrimidin-7-one

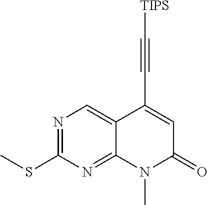

General procedure 9 was applied to 8-methyl-2-(methylsulfanyl)-7-oxopyrido[2,3-d]pyrimidin-5-yl trifluoromethanesulfonate (1.20 g, 3.38 mmol), bis(triphenylphosphine)palladium(II) dichloride (237 mg, 0.338 mmol), triisopropylsilylacetylene (1.23 g, 6.74 mmol), copper(I) iodide (64 mg, 0.336 mmol), N,N-diisopropylethylamine (1.31 g, 10.1 mmol.) in DMF (15 mL). The residue was purified by flash column chromatography eluting with ethyl acetate (5%) in 40-60 petroleum ether to afford the title compound as a light yellow solid (1.10 g, 84%).

(ES, m/z): [M+H]⁺=388.2.

### 2-Methanesulfonyl-8-methyl-5-[2-(triisopropylsilyl)ethynyl]pyrido[2,3-d]pyrimidin-7-one

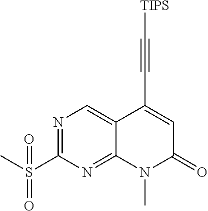

General procedure 10 was applied to 8-methyl-2-(methylsulfanyl)-5-[2-(triisopropylsilyl)ethynyl]pyrido[2,3-d]pyrimidin-7-one (1.10 g, 2.84 mmol) and m-CPBA (1.22 g, 7.09 mmol) in dichloromethane (11.0 mL). The residue was purified by flash column chromatography eluting with ethyl acetate (5%) in 40-60 petroleum ether to afford the title compound as a light yellow solid (1.04 g, 87%).

(ES, m/z): [M+H]⁺=420.2.

### 2-[(4-{[2-(Dimethylamino)ethyl](methyl)amino}-2-methoxyphenyl)amino]-8-methyl-5-[2-(triisopropylsilyl) ethynyl]pyrido[2,3-d]pyrimidin-7-one

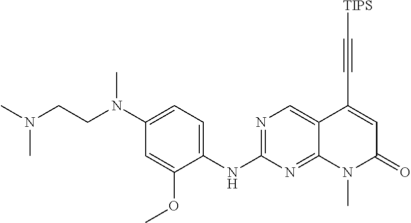

General procedure 13 was applied to 2-methanesulfonyl-8-methyl-5-[2-(triisopropylsilyl)ethynyl]pyrido[2,3-d]pyrimidin-7-one (500 mg, 1.19 mmol) with trifluoroacetic acid (272 mg, 2.38 mmol) and N¹-[2-(dimethylamino)ethyl]-3-methoxy-N¹-methylbenzene-1,4-diamine (319 mg, 1.43 mmol) in 2-butanol (5.0 mL). The resulting solution was diluted with methanol (10 mL) and applied onto a reverse phase column, eluting with acetonitrile (70-100%) in water (0.1% trifluoroacetic acid). The resulting solution was basified to pH 8 with sodium hydrogen carbonate aqueous solution and extracted with ethyl acetate (3×200 mL). The combined organic layers were washed with brine (2×200 mL), dried (Na₂SO₄) and concentrated under reduced pressure to yield the title compound as a red solid (280 mg, 42%).

(ES, m/z): [M+H]⁺=563.3.

### 2-[(4-{[2-(Dimethylamino)ethyl](methyl)amino}-2-methoxyphenyl)amino]-5-ethynyl-8-methylpyrido[2,3-d]pyrimidin-7-one

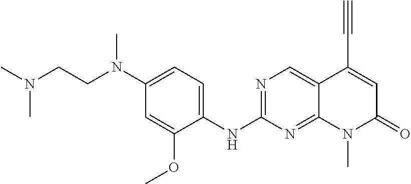 [SMILES: C#Cc1cc(=O)n(C)c2nc(Nc3ccc(N(C)CCN(C)C)cc3OC)ncc12]

General procedure 15 was applied to 2-[(4-{[2-(dimethylamino)ethyl](methyl)amino}-2-methoxyphenyl)amino]-8-methyl-5-[2-(triisopropylsilyl)ethynyl]pyrido[2,3-d]pyrimidin-7-one (150 mg, 0.267 mmol) with potassium fluoride (155 mg, 2.67 mmol) in THF (2.0 mL) and water (1.0 mL). The crude material was purified by reverse phase flash column chromatography eluting with acetonitrile (20-100%) in water (0.1% formic acid) then lyophilized to yield the title compound as a red solid (83 mg, 77%).

(ES, m/z): [M+H]⁺=407.3.

¹H-NMR (300 MHz, DMSO-d₆): δ 8.77 (s, 1H), 8.67 (s, 1H), 8.22 (s, 1H), 7.56 (s, 1H), 6.49 (s, 1H), 6.36 (d, J=2.5 Hz, 1H), 6.28 (dd, J=8.8, 2.6 Hz, 1H), 5.00 (s, 1H), 3.78 (s, 3H), 3.52-3.41 (m, 4H), 2.93 (s, 3H), 2.41 (t, J=7.1 Hz, 2H), 2.20 (s, 6H).

### Example 43

### 2-[(4-{[2-(Dimethylamino)ethyl](methyl)amino}-2-methoxyphenyl)amino]-5-ethynyl-8-methylpyrido[2,3-d]pyrimidin-7-one

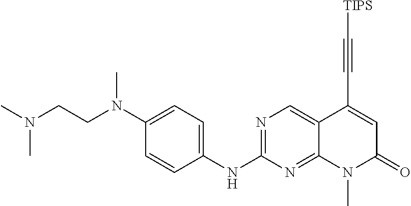

General procedure 13 was applied to 2-methanesulfonyl-8-methyl-5-[2-(triisopropylsilyl)ethynyl]pyrido[2,3-d]pyrimidin-7-one (500 mg, 1.19 mmol) with trifluoroacetic acid (272 mg, 2.38 mmol), N¹-[2-(dimethylamino)ethyl]-N¹-methylbenzene-1,4-diamine (276 mg, 1.43 mmol) in 2-butanol (5.0 mL). The resulting solution was stirred for 3 h at 100° C.

The resulting solution was diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (300 mg, 47%).

(ES, m/z): [M+H]⁺=533.3.

### 2-[(4-{[2-(Dimethylamino)ethyl](methyl)amino}phenyl)amino]-5-ethynyl-8-methylpyrido[2,3-d]pyrimidin-7-one

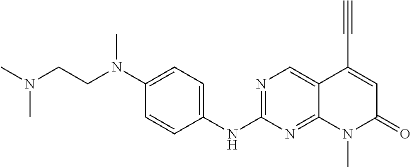 [SMILES: C#Cc1cc(=O)n(C)c2nc(Nc3ccc(N(C)CCN(C)C)cc3)ncc12]

General procedure 15 was applied to 2-[(4-{[2-(dimethylamino)ethyl](methyl)amino}phenyl)amino]-8-methyl-5-[2-(triisopropylsilyl)ethynyl]pyrido[2,3-d]pyrimidin-7-one (450 mg, 0.845 mmol) with potassium fluoride (491 mg, 8.45 mmol) in THF (5.0 mL) and water (0.50 mL). The crude material was purified by reverse phase flash column chromatography eluting with acetonitrile (20-100%) in water (0.1% formic acid) then lyophilized to yield the title compound as a red solid (106 mg, 33%).

(ES, m/z): [M+H]⁺=377.2

¹H-NMR (300 MHz, DMSO-d₆) δ 9.99 (s, 1H), 8.73 (s, 1H), 8.23 (s, 1H), 7.59 (s, 2H), 6.71 (d, J=8.7 Hz, 2H), 6.51 (s, 1H), 5.02 (s, 1H), 3.55 (s, 3H), 3.41 (t, J=7.2 Hz, 2H), 2.89 (s, 3H), 2.43 (t, J=7.1 Hz, 2H), 2.22 (s, 6H).

### Example 44

### 4-(2-Bromo-5-methoxy-4-nitrophenyl)morpholine

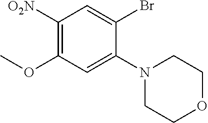 [SMILES: COc1cc(N2CCOCC2)c(Br)cc1[N+](=O)[O-]]

General procedure 11 was applied to 1-bromo-2-fluoro-4-methoxy-5-nitrobenzene (5.00 g, 20.0 mmol) with morpholine (2.09 g, 24.0 mmol) and potassium carbonate (5.53 g, 40.0 mmol) in DMF (50 mL). The residue was triturated with 40-60 petroleum ether:ethyl acetate (60 mL, 1:1), filtered and washed with 40-60 petroleum ether and dried under vacuum to yield the title compound as a yellow solid (4.50 g, 71%).

(ES, m/z): [M+H]⁺=317.0.

### 4-[5-Methoxy-2-(1-methylpyrazol-4-yl)-4-nitrophenyl]morpholine

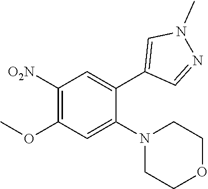 [SMILES: COc1cc(N2CCOCC2)c(-c2cnn(C)c2)cc1[N+](=O)[O-]]

A solution of 4-(2-bromo-5-methoxy-4-nitrophenyl)morpholine (1.50 g, 4.73 mmol, 1.0 eq.), 1-methyl-4-(4,4,5,5-tetramethyl-1,3,2-dioxaborolan-2-yl)pyrazole (1.97 g, 9.46 mmol, 2.0 eq.), potassium carbonate (1.31 g, 9.46 mmol, 2.0 eq.) and [1,1′-bis(diphenylphosphino)ferrocene]dichloropalladium(II) (350 mg, 0.473 mmol, 0.1 eq.), in dioxane (12.0 mL) and water (3.0 mL) was stirred at 70° C. for 1.5 h under a nitrogen atmosphere. The reaction mixture was diluted with dichloromethane (100 mL) and water (100 mL). The dichloromethane layer was separated, and the aqueous layer extracted with dichloromethane (2×100 mL). The combined organic layers were dried (Na₂SO₄) and concentrated under reduced pressure. The residue was purified by flash column chromatography eluting with ethyl acetate (0-75%) in 40-60 petroleum ether to yield the title compound as a yellow solid (1.00 g, 66%).

(ES, m/z): [M+H]⁺=319.1.

### 2-Methoxy-5-(1-methylpyrazol-4-yl)-4-(morpholin-4-yl)aniline

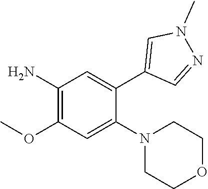 [SMILES: COc1cc(N2CCOCC2)c(-c2cnn(C)c2)cc1N]

General procedure 12 was applied to 4-[5-methoxy-2-(1-methylpyrazol-4-yl)-4-nitrophenyl]morpholine (1.00 g, 3.14 mmol) with palladium on carbon 5 wt. % (100 mg, 0.939 mmol) in methanol (15 mL) to yield the title compound as a grey solid (700 mg, 77%).

(ES, m/z): [M+H]⁺=289.2.

### 2-{[2-Methoxy-5-(1-methylpyrazol-4-yl)-4-(morpholin-4-yl)phenyl]amino}-8-methyl-5-[2-(triisopropylsilyl)ethynyl]pyrido[2,3-d]pyrimidin-7-one

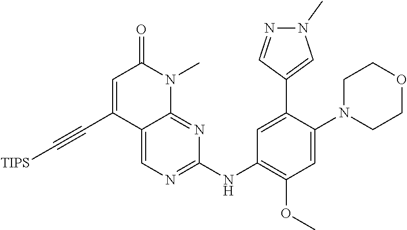

General procedure 13 was applied to 2-methanesulfonyl-8-methyl-5-[2-(triisopropylsilyl)ethynyl]pyrido[2,3-d]pyrimidin-7-one (350 mg, 0.834 mmol) with 2-methoxy-5-(1-methylpyrazol-4-yl)-4-(morpholin-4-yl)aniline and trifluoroacetic acid in 2-butanol (5.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (300 mg, 57%).

(ES, m/z): [M+H]⁺=628.3.

### 5-Ethynyl-2-{[2-methoxy-5-(1-methylpyrazol-4-yl)-4-(morpholin-4-yl)phenyl]amino}-8-methylpyrido[2,3-d]pyrimidin-7-one

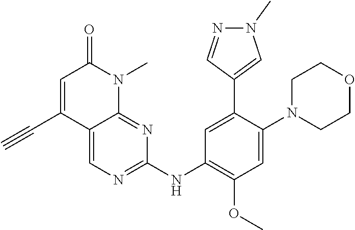 [SMILES: C#Cc1cc(=O)n(C)c2nc(Nc3cc(-c4cnn(C)c4)c(N4CCOCC4)cc3OC)ncc12]

General procedure 15 was applied to 2-{[2-methoxy-5-(1-methylpyrazol-4-yl)-4-(morpholin-4-yl)phenyl]amino}-8-methyl-5-[2-(triisopropylsilyl)ethynyl]pyrido[2,3-d]pyrimidin-7-one (350 mg, 0.557 mmol), with potassium fluoride (324 mg, 5.57 mmol) in THF (3.0 mL). The resulting solution was stirred for 2 h at 50° C., applied onto a reverse phase column, eluting with acetonitrile (20-100%) in water (0.1% formic acid). The desired fractions were collected and concentrated under reduced pressure to remove most of the acetonitrile then lyophilized to yield the title compound as a yellow solid (106 mg, 40%).

(ES, m/z): [M+H]⁺=472.2.

¹H-NMR (300 MHz, DMSO-d₆) δ 8.82 (s, 1H), 8.72 (s, 1H), 8.11 (s, 1H), 7.88 (s, 1H), 6.81 (s, 1H), 6.54 (s, 1H), 5.01 (s, 1H), 3.88 (s, 6H), 3.73 (s, 4H), 3.49 (s, 3H), 3.32 (s, 3H), 2.84 (s, 4H).

### Example 45

### 8-Methyl-2-{[4-(4-methylpiperazin-1-yl)phenyl]amino}-5-[2-(triisopropylsilyl)ethynyl]pyrido[2,3-d]pyrimidin-7-one

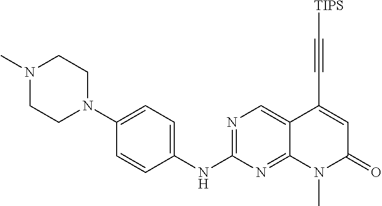

General procedure 13 was applied to 2-methanesulfonyl-8-methyl-5-[2-(triisopropylsilyl)ethynyl]pyrido[2,3-d]pyrimidin-7-one (500 mg, 1.19 mmol) with 4-(4-methylpiperazin-1-yl)aniline (273 mg, 1.43 mmol) and trifluoroacetic acid (272 mg, 2.38 mmol) in 2-butanol (5.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (290 mg, 46%).

(ES, m/z): [M+H]⁺=531.3.

### 5-Ethynyl-8-methyl-2-{[4-(4-methylpiperazin-1-yl)phenyl]amino}pyrido[2,3-d]pyrimidin-7-one

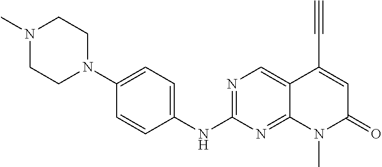 [SMILES: C#Cc1cc(=O)n(C)c2nc(Nc3ccc(N4CCN(C)CC4)cc3)ncc12]

General procedure 15 was applied to 8-methyl-2-{[4-(4-methylpiperazin-1-yl)phenyl]amino}-5-[2-(triisopropylsilyl)ethynyl]pyrido[2,3-d]pyrimidin-7-one (150 mg, 0.283 mmol) with potassium fluoride (164 mg, 2.83 mmol) in THF (2.0 mL) and water (1.0 mL). The resulting solution was stirred for 16 h at 80° C. and applied onto a reverse phase column directly, eluting with acetonitrile (20-100%) in water (0.1% formic acid). The desired fractions were collected and concentrated under reduced pressure to remove most of the acetonitrile then lyophilized to yield the title compound as a yellow solid (27.0 mg, 25%).

(ES, m/z): [M+H]⁺=375.2

¹H-NMR (300 MHz, DMSO-d₆) δ 10.07 (s, 1H), 8.75 (s, 1H), 8.15 (s, 1H), 7.64 (d, J=8.4 Hz, 2H), 6.98-6.91 (m, 2H), 6.54 (s, 1H), 5.02 (s, 1H), 3.55 (s, 3H), 3.11 (t, J=5.0 Hz, 4H), 2.48 (s, 1H), 2.25 (s, 3H).

### Example 46

### 2-({4-[2-(Dimethylamino)ethoxy]phenyl}amino)-8-methyl-5-[2-(triisopropylsilyl)ethynyl]pyrido[2,3-d]pyrimidin-7-one

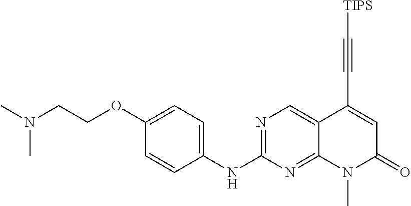

General procedure 13 was applied to 2-methanesulfonyl-8-methyl-5-[2-(triisopropylsilyl)ethynyl]pyrido[2,3-d]pyrimidin-7-one (500 mg, 1.19 mmol) with trifluoroacetic acid (272 mg, 2.38 mmol) and 4-[2-(dimethylamino)ethoxy]aniline (258 mg, 1.43 mmol) in 2-butanol (5.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (280 mg, 45%).

(ES, m/z): [M+H]⁺=520.3.

### 2-({4-[2-(Dimethylamino)ethoxy]phenyl}amino)-5-ethynyl-8-methylpyrido[2,3-d]pyrimidin-7-one

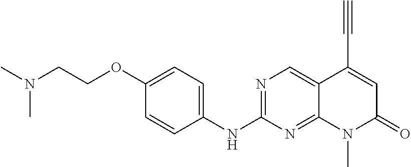 [SMILES: C#Cc1cc(=O)n(C)c2nc(Nc3ccc(OCCN(C)C)cc3)ncc12]

General procedure 15 was applied to 2-({4-[2-(dimethylamino)ethoxy]phenyl}amino)-8-methyl-5-[2-(triisopropylsilyl)ethynyl]pyrido[2,3-d]pyrimidin-7-one (150 mg, 0.289 mmol) with potassium fluoride (167 mg, 2.89 mmol) in THF (2.0 mL) and water (0.50 mL). The resulting solution was stirred for 16 h at 80° C. and applied onto a reverse phase column, eluting with acetonitrile (20-100%) in water (0.1% formic acid).

The desired fractions were collected and concentrated under reduced pressure to remove most of the acetonitrile then lyophilized to yield the title compound as a yellow solid (61.0 mg, 58%).

(ES, m/z): [M+H]⁺=364.2.

¹H-NMR (300 MHz, DMSO-d₆) δ 10.12 (s, 1H), 8.77 (s, 1H), 7.69 (d, J=8.5 Hz, 2H), 6.94 (d, J=6.9 Hz, 2H), 6.55 (s, 1H), 5.03 (s, 1H), 4.03 (t, J=5.8 Hz, 2H), 3.55 (s, 3H), 3.52 (s, 1H), 2.62 (t, J=5.8 Hz, 2H), 2.22 (s, 6H).

### Example 47

### 2-[(4-{[2-(Dimethylamino)ethyl](methyl)amino}-3-(1-methylpyrazol-4-yl)phenyl)amino]-8-methyl-5-[2-(trimethylsilyl)ethynyl]pyrido[2,3-d]pyrimidin-7-one

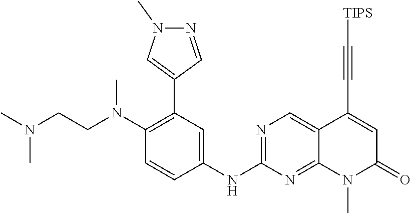

General procedure 13 was applied to 2-methanesulfonyl-8-methyl-5-[2-(trimethylsilyl)ethynyl]pyrido[2,3-d]pyrimidin-7-one (500 mg, 1.491 mmol) with trifluoroacetic acid (272 mg, 2.38 mmol) and N¹-[2-(dimethylamino)ethyl]-N¹-methyl-2-(1-methylpyrazol-4-yl)benzene-1,4-diamine (489 mg, 1.79 mmol) in 2-butanol (5.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as an orange solid (300 mg, 38%).

(ES, m/z): [M+H]⁺=613.4.

### 2-[(4-{[2-(Dimethylamino)ethyl](methyl)amino}-3-(1-methylpyrazol-4-yl)phenyl)amino]-5-ethynyl-8-methylpyrido[2,3-d]pyrimidin-7-one

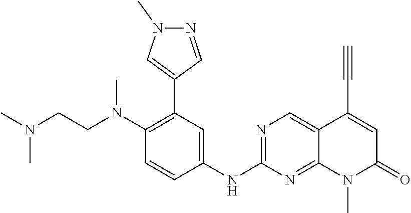 [SMILES: C#Cc1cc(=O)n(C)c2nc(Nc3ccc(N(C)CCN(C)C)c(-c4cnn(C)c4)c3)ncc12]

General procedure 15 was applied to 2-[(4-{[2-(dimethylamino)ethyl](methyl)amino}-3-(1-methylpyrazol-4-yl)phenyl)amino]-8-methyl-5-[2-(triisopropylsilyl)ethynyl]pyrido[2,3-d]pyrimidin-7-one (300 mg, 0.489 mmol), with potassium fluoride (284 mg, 4.89 mmol) in THF (3.0 mL) and water (1.0 mL). The resulting solution was stirred for 6 h at 70° C. and applied onto a reverse phase column directly, eluting with acetonitrile (20-100%) in water (0.1% formic acid). The desired fractions were collected and concentrated under reduced pressure to remove most of the acetonitrile then lyophilized to yield the title compound as a brown solid (108 mg, 48%).

(ES, m/z): [M+H]⁺=457.2.

¹H-NMR (300 MHz, DMSO-d₆) δ 10.14 (s, 1H), 8.80 (s, 1H), 8.21 (d, J=9.1 Hz, 2H), 7.92-7.85 (m, 2H), 7.60 (dd, J=8.8, 2.5 Hz, 1H), 7.18 (d, J=8.7 Hz, 1H), 6.57 (s, 1H), 5.05 (s, 1H), 3.88 (s, 3H), 3.58 (s, 3H), 2.95 (dd, J=8.3, 5.9 Hz, 2H), 2.55 (s, 3H), 2.41 (t, J=7.1 Hz, 2H), 2.15 (s, 6H).

### Example 48

### 2-[(2-Methoxyphenyl)amino]-8-methyl-5-[2-(triisopropylsilyl)ethynyl]pyrido[2,3-d]pyrimidin-7-one

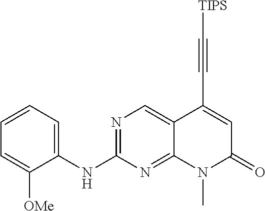

General procedure 13 was applied to 2-methanesulfonyl-8-methyl-5-[2-(triisopropylsilyl)ethynyl]pyrido[2,3-d]pyrimidin-7-one (150 mg, 0.357 mmol) with trifluoroacetic acid (40.7 mg, 0.357 mmol) and o-anisidine (44.0 mg, 0.357 mmol) in 2-butanol (3.0 mL). The resulting solution was stirred for 16 h at 110° C., diluted with methanol (15 mL) and applied onto a reverse phase column, eluting with acetonitrile (70-100%) in water (0.1% NH₄HCO₃) to yield the title compound as a light yellow solid (65.0 mg, 39%).

(ES, m/z): [M+H]⁺=463.

### 5-Ethynyl-2-[(2-methoxyphenyl) amino]-8-methylpyrido[2,3-d]pyrimidin-7-one

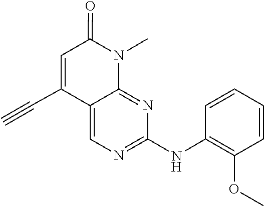 [SMILES: C#Cc1cc(=O)n(C)c2nc(Nc3ccccc3OC)ncc12]

General procedure 15 was applied 2-[(2-methoxyphenyl)amino]-8-methyl-5-[2-(triisopropylsilyl)ethynyl]pyrido[2,3-d]pyrimidin-7-one (80.0 mg, 0.173 mmol) with potassium fluoride (100 mg, 0.0173 mmol) in THF (5.0 mL) and water (2.0 mL). The resulting solution was stirred for 2 days at 40° C. and applied onto a reverse phase column, eluting with acetonitrile (20-100%) in water (0.1% NH₄HCO₃). The desired fractions were collected and concentrated under reduced pressure to remove most of the acetonitrile then lyophilized to yield the title compound as a light yellow solid (15.0 mg, 28%).

(ES, m/z): [M+H]⁺=307.

¹H NMR (300 MHz, CDCl₃) δ 8.84 (s, 1H), 8.39 (d, J=8.2 Hz, 1H), 7.18-7.02 (m, 2H), 6.99 (d, J=8.1 Hz, 1H), 6.72 (s, 1H), 3.98 (s, 3H), 3.72 (d, J=14.7 Hz, 4H).

### Example 49

### 2-{[2-Methoxy-4-(4-methylpiperazin-1-yl)phenyl]amino}-8-methyl-5-[2-(triisopropylsilyl)ethynyl]pyrido[2,3-d]pyrimidin-7-one

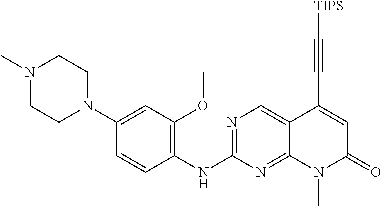

General procedure 13 was applied to 2-methanesulfonyl-8-methyl-5-[2-(triisopropylsilyl)ethynyl]pyrido[2,3-d]pyrimidin-7-one (500 mg, 1.19 mmol), trifluoroacetic acid (272 mg, 2.38 mmol) and 2-methoxy-4-(4-methylpiperazin-1-yl)aniline (316 mg, 1.43 mmol) in 2-butanol (5.0 mL). The resulting solution was stirred for 3 h at 100° C. and purified by reverse phase flash column chromatography eluting with acetonitrile (70-100%) in water (0.1% trifluoroacetic acid) to yield the title compound as a yellow solid (300 mg, 45%).

(ES, m/z): [M+H]⁺=561.3.

### 5-Ethynyl-2-{[2-methoxy-4-(4-methylpiperazin-1-yl)phenyl]amino}-8-methylpyrido[2,3-d]pyrimidin-7-one

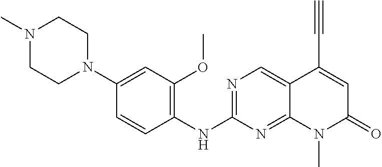 [SMILES: C#Cc1cc(=O)n(C)c2nc(Nc3ccc(N4CCN(C)CC4)cc3OC)ncc12]

General procedure 15 was applied to 2-{[2-methoxy-4-(4-methylpiperazin-1-yl)phenyl]amino}-8-methyl-5-[2-(triisopropylsilyl)ethynyl]pyrido[2,3-d]pyrimidin-7-one (150 mg, 0.267 mmol) and potassium fluoride (155 mg, 2.67 mmol) in THF (2.0 mL) and water (0.50 mL). The reaction was stirred for 16 h at 80° C. and purified by reverse phase flash column chromatography, eluting with acetonitrile (20-100%) in water (0.1% NH₄HCO₃). The desired fractions were collected and concentrated under reduced pressure to remove most of the acetonitrile, then lyophilized to yield the title compound as a red solid (22.7 mg, 21%).

(ES, m/z): [M+H]⁺=405.2.

¹H-NMR (300 MHz, DMSO-d₆) δ 8.81 (s, 1H), 8.70 (s, 1H), 7.63 (s, 1H), 7.61 (m, 1H), 6.65 (d, J=2.5 Hz, 1H), 6.53 (d, J=7.9 Hz, 2H), 5.01 (s, 1H), 3.81 (s, 3H), 3.47 (s, 3H), 3.16 (t, J=5.0 Hz, 4H), 2.53 (s, 1H), 2.25 (s, 3H).

### Example 50

### N-(2-Fluoro-5-nitrophenyl)acetamide

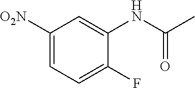

A solution of 2-fluoro-5-nitroaniline (1.60 g, 10.2 mmol, 1.00 eq.) in acetic anhydride (8.0 mL) was stirred for 1 h at room temperature. The precipitated solids were collected by filtration and washed with water (2×20 mL) to yield the title compound as a white solid (1.50 g, 74%).

(ES, m/z): [M+H]⁺=199.0.

### N-[2-(4-Methylpiperazin-1-yl)-5-nitrophenyl]acetamide

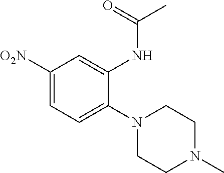 [SMILES: CC(=O)Nc1cc([N+](=O)[O-])ccc1N1CCN(C)CC1]

General procedure 11 was applied to N-(2-fluoro-5-nitrophenyl)acetamide (1.50 g, 7.57 mmol) with 1-methylpiperazine (910 mg, 9.08 mmol) and potassium carbonate (2.51 g, 18.2 mmol) in DMF (15 mL) to yield the title compound as a white solid (1.50 g, 71%).

(ES, m/z): [M+H]⁺=279.1.

### N-[5-Amino-2-(4-methylpiperazin-1-yl)phenyl]acetamide

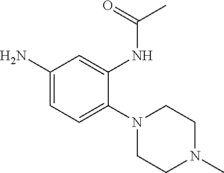 [SMILES: CC(=O)Nc1cc(N)ccc1N1CCN(C)CC1]

General procedure 12 was applied to N-[2-(4-methylpiperazin-1-yl)-5-nitrophenyl]acetamide (1.50 g, 5.39 mmol) with palladium on carbon 5 wt. % (110 mg, 1.08 mmol) in methanol (15 mL) to yield the title compound as a dark green solid (1.20 g, 90%).

(ES, m/z): [M+H]⁺=249.2.

### N-[5-({8-Methyl-7-oxo-5-[2-(triisopropylsilyl)ethynyl]pyrido[2,3-d]pyrimidin-2-yl}amino)-2-(4-methylpiperazin-1-yl)phenyl]acetamide

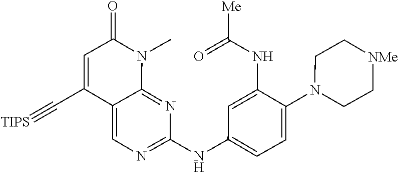

General procedure 13 was applied to 2-methanesulfonyl-8-methyl-5-[2-(triisopropylsilyl)ethynyl]pyrido[2,3-d]pyrimidin-7-one (500 mg, 1.19 mmol) with trifluoroacetic acid (272 mg, 2.38 mmol) and N-[5-amino-2-(4-methylpiperazin-1-yl)phenyl]acetamide (355 mg, 1.43 mmol) in 2-butanol (5.0 mL). The solution mixture was stirred for 4 h at 100° C. and purified by reverse phase flash column chromatography, eluting with acetonitrile (30-60%) in water (0.1% formic acid) to yield the title compound as an orange solid (250 mg, 36%).

(ES, m/z): [M+H]⁺=588.3.

### N-[5-({5-Ethynyl-8-methyl-7-oxopyrido[2,3-d]pyrimidin-2-yl}amino)-2-(4-methylpiperazin-1-yl)phenyl]acetamide

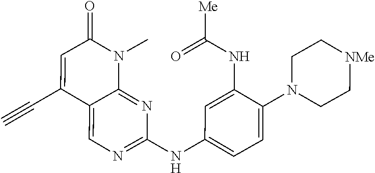 [SMILES: C#Cc1cc(=O)n(C)c2nc(Nc3ccc(N4CCN(C)CC4)c(NC(C)=O)c3)ncc12]

General procedure 15 was applied to N-[5-({8-methyl-7-oxo-5-[2-(triisopropylsilyl)ethynyl]pyrido[2,3-d]pyrimidin-2-yl}amino)-2-(4-methylpiperazin-1-yl)phenyl]acetamide (250 mg, 0.425 mmol) with potassium fluoride (247 mg, 4.25 mmol) in DMF (2.0 mL), water (50 μL) and THF (2.0 mL). The resulting mixture was stirred for 2 h at 80° C. and purified by reverse phase flash column chromatography, eluting with acetonitrile (30-60%) in water (0.1% formic acid) to yield the title compound as a yellow solid (28 mg, 14%).

(ES, m/z): [M+H]⁺=432.2.

¹H NMR (400 MHz, DMSO-d₆) δ 10.20 (s, 1H), 8.80 (s, 1H), 8.77 (s, 1H), 8.54 (s, 1H), 7.52-7.36 (m, 1H), 7.15 (d, J=8.7 Hz, 1H), 6.57 (s, 1H), 5.04 (s, 1H), 3.58 (s, 3H), 2.82 (t, J=4.7 Hz, 4H), 2.59 (s, 4H), 2.30 (s, 3H), 2.12 (s, 3H).

### Example 51

### N-(2-Fluoro-5-nitrophenyl)propanamide

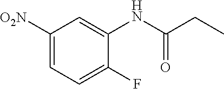

A solution of 2-fluoro-5-nitroaniline (1.50 g, 9.61 mmol, 1.0 eq.) in propionic anhydride (8.0 mL) was stirred for 1 h at room temperature. The precipitated solids were collected by filtration and washed with water (2×20 mL) to yield the title compound as a white solid (1.50 g, 74%).

(ES, m/z): [M+H]⁺=213.1.

### N-[2-(4-Methylpiperazin-1-yl)-5-nitrophenyl]propanamide

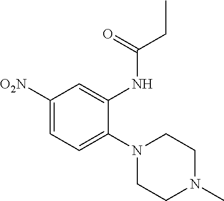 [SMILES: CCC(=O)Nc1cc([N+](=O)[O-])ccc1N1CCN(C)CC1]

General procedure 11 was applied to N-(2-fluoro-5-nitrophenyl)propanamide (1.50 g, 7.07 mmol) with 1-methylpiperazine (910 mg, 9.08 mmol) and potassium carbonate (2.34 g, 16.9 mmol) in DMF (15 mL) to yield the title compound as a white solid (2.00 g, 97%).

(ES, m/z): [M+H]⁺=293.2.

### N-[5-Amino-2-(4-methylpiperazin-1-yl)phenyl]propanamide

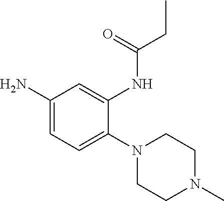 [SMILES: CCC(=O)Nc1cc(N)ccc1N1CCN(C)CC1]

General procedure 12 was applied to N-[2-(4-methylpiperazin-1-yl)-5-nitrophenyl]propanamide (2.00 g, 6.84 mmol) with palladium on carbon 5 wt. % in methanol (20 mL) to yield the title compound as a dark green solid (1.80 g, 95%).

(ES, m/z): [M+H]⁺=262.2.

### N-[5-({8-Methyl-7-oxo-5-[2-(triisopropylsilyl)ethynyl]pyrido[2,3-d]pyrimidin-2-yl}amino)-2-(4-methylpiperazin-1-yl)phenyl]propanamide

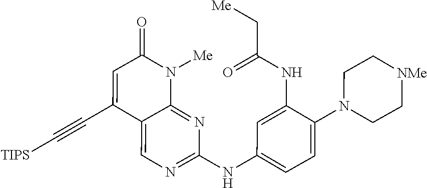

General procedure 13 was applied to 2-methanesulfonyl-8-methyl-5-[2-(triisopropylsilyl)ethynyl]pyrido[2,3-d]pyrimidin-7-one (1.00 g, 2.38 mmol) with N-[5-amino-2-(4-methylpiperazin-1-yl)phenyl]propanamide (750 mg, 2.86 mmol) and trifluoroacetic acid (540 mg, 4.76 mmol) in 2-butanol (10 mL). The solution mixture was stirred for 4 h at 100° C. and purified by reverse phase flash column chromatography, eluting with acetonitrile (30-60%) in water (0.1% formic acid) to yield the title compound as an orange solid (600 mg, 42%).

(ES, m/z): [M+H]⁺=602.4.

### N-[5-({5-Ethynyl-8-methyl-7-oxopyrido[2,3-d]pyrimidin-2-yl}amino)-2-(4-methylpiperazin-1-yl)phenyl]propanamide

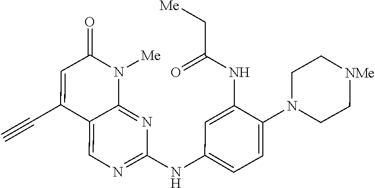 [SMILES: C#Cc1cc(=O)n(C)c2nc(Nc3ccc(N4CCN(C)CC4)c(NC(=O)CC)c3)ncc12]

General procedure 15 was applied to N-[5-({8-methyl-7-oxo-5-[2-(triisopropylsilyl)ethynyl]pyrido[2,3-d]pyrimidin-2-yl}amino)-2-(4-methylpiperazin-1-yl)phenyl]propanamide (600 mg, 0.997 mmol) and potassium fluoride (579 mg, 9.97 mmol) in THF (6.0 mL) and methanol (6.0 mL). The resulting mixture was stirred for 2 h at 80° C. and cooled to room temperature. Water was added and the precipitate was collected by filtration, washing with water/acetonitrile (1:1, 3×5 mL) to yield the title compound as a yellow solid (170 mg, 37%).

(ES, m/z): [M+H]⁺=446.0.

¹H NMR (300 MHz, DMSO-d₆) δ 10.20 (s, 1H), 8.78 (d, J=3.4 Hz, 2H), 8.61 (s, 1H), 7.41 (dd, J=8.7, 2.6 Hz, 1H), 7.16 (d, J=8.7 Hz, 1H), 6.57 (s, 1H), 5.04 (s, 1H), 2.80 (t, J=4.7 Hz, 4H), 2.50 (t, J=4.7 Hz, 4H), 2.42 (q, J=7.6 Hz, 2H), 2.25 (s, 3H), 1.14 (t, J=7.5 Hz, 3H).

### Example 52

### 3-Bromo-N-[2-(dimethylamino)ethyl]-N-methylaniline

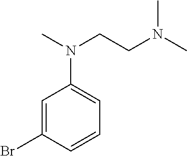

A solution of [2-(dimethylamino)ethyl](methyl)amine (6.00 g, 58.7 mmol, 1.0 eq.), 1,3-dibromobenzene (14.5 g, 61.6 mmol, 1.05 eq.), tris(dibenzylideneacetone)dipalladium(0) (2.69 g, 2.94 mmol, 0.05 eq.), XantPhos (3.40 g, 5.87 mmol, 0.1 eq.) and potassium tert-butoxide (16.5 g, 147 mmol, 2.5 eq.) in dioxane (60 mL) was stirred for 2 h at 100° C. The reaction mixture was quenched with water (100 mL) and extracted with ethyl acetate (2×100 mL). The combined organic layers were dried (Na₂SO₄) and concentrated under reduced pressure. The crude material was purified by flash column chromatography, eluting with methanol (10%) in dichloromethane to yield the title compound as a red liquid (5.00 g, 33%).

(ES, m/z): [M+H]⁺=257.

### tert-Butyl N-(3-{[2-(dimethylamino)ethyl](methyl)amino}phenyl)carbamate

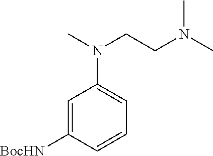 [SMILES: CN(C)CCN(C)c1cccc(NC(=O)OC(C)(C)C)c1]

A solution of 3-bromo-N-[2-(dimethylamino)ethyl]-N-methylaniline (2.00 g, 7.77 mmol, 1.0 eq.), tert-butyl carbamate (1.37 g, 11.6 mmol, 1.5 eq.), tris(dibenzylideneacetone)dipalladium(0) (356 mg, 0.389 mmol, 0.05 eq.), tBuXPhos (330.24 mg, 0.778 mmol, 0.1 eq.) and potassium tert-butoxide (2.18 g, 19.442 mmol, 2.5 eq.) in toluene (20 mL) was stirred for 4 h at 80° C. The reaction was quenched with water (100 mL) and extracted with ethyl acetate (2×100 mL), dried (Na₂SO₄) and concentrated under reduced pressure. The crude material was purified by flash column chromatography, eluting with methanol (10%) in dichloromethane to yield the title compound as a brown oil (2.00 g, 88%).

(ES, m/z): [M+H]⁺=294.

### N¹-[2-(Dimethylamino)ethyl]-N¹-methylbenzene-1,3-diamine

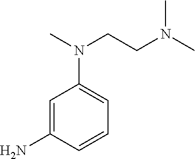

A solution of tert-butyl N-(3-{[2-(dimethylamino)ethyl](methyl)amino}phenyl)carbamate (1.80 g, 6.14 mmol, 1.0 eq.) in 4M hydrogen chloride in 1,4-dioxane (20 mL) was stirred for 3 h at 30° C. The reaction was concentrated under reduced pressure to yield the title compound as a white solid (1.15 g, 97%).

(ES, m/z): [M+H]⁺=194.

### 2-[(3-{[2-(Dimethylamino)ethyl](methyl)amino}phenyl)amino]-8-methyl-5-[2-(triisopropylsilyl)ethynyl]pyrido[2,3-d]pyrimidin-7-one

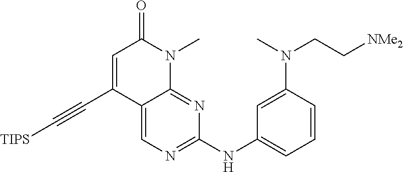

General procedure 13 was applied to 2-methanesulfonyl-8-methyl-5-[2-(triisopropylsilyl)ethynyl]pyrido[2,3-d]pyrimidin-7-one (400 mg, 0.953 mmol) with trifluoroacetic acid (217 mg, 1.91 mmol) and N¹-[2-(dimethylamino)ethyl]-N¹-methylbenzene-1,3-diamine (221 mg, 1.14 mmol) in 2-butanol (4.0 mL). The resulting solution was stirred for 3 h at 100° C., diluted with methanol (10 mL) and applied onto a reverse phase column, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (280 mg, 55%).

(ES, m/z): [M+H]⁺=533.

### 2-[(3-{[2-(Dimethylamino)ethyl](methyl)amino}phenyl)amino]-5-ethynyl-8-methylpyrido[2,3-d]pyrimidin-7-one

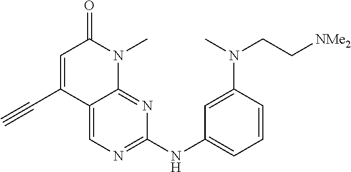 [SMILES: C#Cc1cc(=O)n(C)c2nc(Nc3cccc(N(C)CCN(C)C)c3)ncc12]

General procedure 15 was applied to 2-[(3-{[2-(dimethylamino)ethyl](methyl)amino}phenyl)amino]-8-methyl-5-[2-(triisopropylsilyl)ethynyl]pyrido[2,3-d]pyrimidin-7-one (300 mg, 0.563 mmol) and potassium fluoride (327 mg, 5.63 mmol) in THF (3.0 mL), DMF (3.0 mL) and water (0.50 mL). The resulting solution was stirred for 2 h at 70° C. and purified by reverse phase flash column chromatography, eluting with acetonitrile (20-100%) in water (0.1% formic acid). The desired fractions were collected and concentrated under reduced pressure to remove most of the acetonitrile and lyophilized to yield the title compound as a yellow solid (119 mg, 56%).

(ES, m/z): [M+H]⁺=377

¹H-NMR (300 MHz, DMSO-d₆) δ 10.08 (s, 1H), 8.80 (s, 1H), 7.28-7.16 (m, 3H), 6.65-6.54 (m, 2H), 5.06 (s, 1H), 3.68-3.62 (m, 2H), 3.60 (s, 3H), 3.29-3.23 (m, 2H), 2.94 (s, 3H), 2.85 (s, 6H).

### Example 53

### 2-(Dimethylamino)-N-methyl-N-[3-({8-methyl-7-oxo-5-[2-(triisopropylsilyl)ethynyl]pyrido[2,3-d]pyrimidin-2-yl}amino)phenyl]acetamide

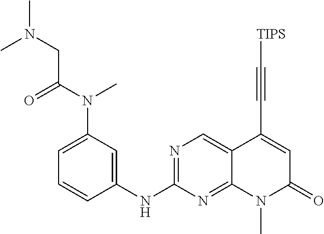

General procedure 13 was applied to 2-methanesulfonyl-8-methyl-5-[2-(triisopropylsilyl)ethynyl]pyrido[2,3-d]pyrimidin-7-one (800 mg, 1.91 mmol) with trifluoroacetic acid (435 mg, 3.81 mmol) and N-(3-aminophenyl)-2-(dimethylamino)-N-methylacetamide (474 mg, 2.29 mmol) in 2-butanol (8.0 mL). The resulting solution was stirred for 3 h at 100° C., diluted with methanol (10 mL) and purified by reverse phase flash column chromatography, eluting with acetonitrile (70-100%) in water (0.1% trifluoroacetic acid). The collected fractions were basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (500 mg, 48%).

(ES, m/z): [M+H]⁺=547.

### 2-(Dimethylamino)-N-[3-({5-ethynyl-8-methyl-7-oxopyrido[2,3-d]pyrimidin-2-yl}amino)phenyl]-N-methylacetamide

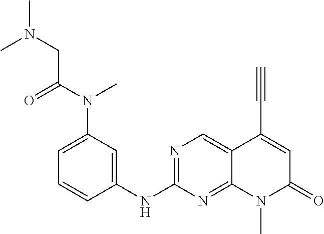 [SMILES: C#Cc1cc(=O)n(C)c2nc(Nc3cccc(N(C)C(=O)CN(C)C)c3)ncc12]

General procedure 15 was applied to 2-(dimethylamino)-N-methyl-N-[3-({8-methyl-7-oxo-5-[2-(triisopropylsilyl)ethynyl]pyrido[2,3-d]pyrimidin-2-yl}amino)phenyl]acetamide (300 mg, 0.549 mmol) with potassium fluoride (319 mg, 5.49 mmol) in THF (3.0 mL), DMF (3.0 mL) and water (0.50 mL). The resulting solution was stirred for 2 h at 70° C. and applied onto a reverse phase column, eluting with acetonitrile (20-100%) in water (0.1% formic acid). The collected fractions were concentrated under reduced pressure to remove most of the acetonitrile then lyophilized to yield the title compound as a brown solid (33.7 mg, 16%).

(ES, m/z): [M+H]⁺=391.

¹H-NMR (300 MHz, DMSO-d₆) δ 11.78 (s, 1H), 10.35 (s, 1H), 8.82 (s, 1H), 8.58-8.45 (m, 2H), 7.43 (d, J=7.6 Hz, 1H), 7.27 (d, J=20.2 Hz, 2H), 6.61 (s, 1H), 5.07 (s, 1H), 4.52-4.45 (m, 2H), 3.63 (s, 3H), 3.40 (s, 6H), 1.24 (s, 1H).

### Example 54

### Ethyl 2-(methylthio)-4-((3-nitrophenyl)amino)pyrimidine-5-carboxylate

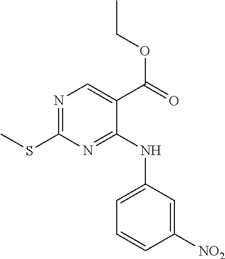 [SMILES: CCOC(=O)c1cnc(SC)nc1Nc1cccc([N+](=O)[O-])c1]

Ethyl 4-chloro-2-(methylthio)pyrimidine-5-carboxylate (10.0 g, 43.0 mmol, 1.0 eq.), 3-nitroaniline (11.9 g, 86.0 mmol, 2.0 eq.) and triethylamine (14.9 mL, 107 mmol, 2.5 eq.) were dissolved in THF (150 mL) and stirred at 70° C. overnight. The resulting solid was collected by vacuum filtration and washed with THF (500 mL) to obtain the title compound as a beige solid (10.2 g, 30.5 mmol, 71%).

(ES, m/z): [M+H]⁺=335.2

¹H NMR (500 MHz, DMSO-d₆) δ 1.36 (3H, t, J=7.1 Hz), 2.56 (3H, s), 4.38 (2H, q, J=7.1 Hz), 7.67 (1H, t, J=8.2 Hz), 7.93 (1H, dd, J=8.2, 2.2 Hz), 8.00 (1H, dd, J=8.2, 2.3 Hz), 8.79 (1H, s), 8.98 (1H, t, J=2.3 Hz), 10.49 (1H, s).

### 2-(Methylthio)-4-((3-nitrophenyl)amino)pyrimidine-5-carboxylic acid

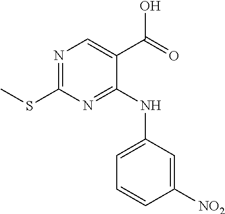 [SMILES: CSc1ncc(C(=O)O)c(Nc2cccc([N+](=O)[O-])c2)n1]

General Procedure 3 was applied to ethyl 2-(methylthio)-4-((3-nitrophenyl)amino)pyrimidine-5-carboxylate (10.0 g, 29.8 mmol) with 1 M aqueous sodium hydroxide (194 mL, 194 mmol) in THF (194 mL) to yield the title compound as a white solid (8.80 g, 28.7 mmol, 96%).

(ES, m/z): [M+H]⁺=307.1.

¹H NMR (500 MHz, DMSO-d₆) δ 2.58 (3H, s), 7.63 (1H, t, J=7.8 Hz), 7.83 (1H, dd, J=8.2, 2.1 Hz), 7.92 (1H, ddd, J=7.9, 2.3, 0.9 Hz), 8.70 (1H, s), 9.06 (1H, t, J=2.2 Hz), 12.36 (1H, s), COOH not visualised.

### 2-(Methylthio)-4-((3-nitrophenyl)amino)pyrimidine-5-carbonyl chloride

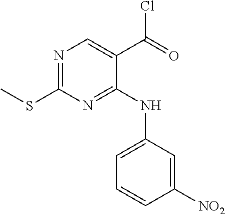 [SMILES: CSc1ncc(C(=O)Cl)c(Nc2cccc([N+](=O)[O-])c2)n1]

To a solution of 2-(methylthio)-4-((3-nitrophenyl)amino)pyrimidine-5-carboxylic acid (6.00 g, 19.6 mmol) in toluene (5.0 mL) was added thionyl chloride (15.0 mL). The resulting mixture was stirred at 95° C. for 4 h. The solvent was evaporated to yield the title compound as a white solid (6.30 g, 19.4 mmol, 99%), which was used without further purification.

### Ethyl 3-(2-(methylthio)-4-((3-nitrophenyl)amino)pyrimidin-5-yl)-3-oxopropanoate

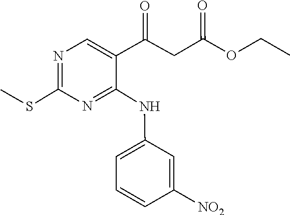 [SMILES: CCOC(=O)CC(=O)c1cnc(SC)nc1Nc1cccc([N+](=O)[O-])c1]

General Procedure 5 was applied to a solution of 2-(methylthio)-4-((3-nitrophenyl)amino)pyrimidine-5-carbonyl chloride (6.30 g, 19.4 mmol) in THF (19.4 mL), with 1 M LiHMDS in THF (67.9 mL, 67.9 mmol), EtOAc (3.79 mL, 38.8 mmol) and THF (58.2.0 mL) to yield the title compound was obtained as a light brown solid (5.89 g, 15.6 mmol, 80%).

(ES, m/z): [M+H]⁺=377.2.

¹H NMR (500 MHz, DMSO-d₆) δ 1.21 (3H, t, J=7.1 Hz), 2.56 (3H, s), 4.15 (2H, q, J=7.1 Hz), 4.28 (2H, s), 7.62-7.68 (1H, m), 7.93 (1H, ddd, J=8.2, 2.2, 0.9 Hz), 7.99 (1H, ddd, J=8.2, 2.3, 0.9 Hz), 8.94 (1H, s), 8.98 (1H, t, J=2.2 Hz), 11.22 (1H, s).

### 5-Hydroxy-2-(methylthio)-8-(3-nitrophenyl)pyrid[2,3-d]pyrimidin-7(8H)-one

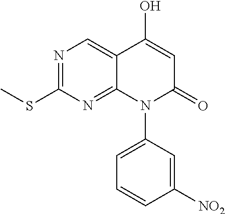 [SMILES: CSc1ncc2c(O)cc(=O)n(-c3cccc([N+](=O)[O-])c3)c2n1]

General Procedure 6 was applied to ethyl 3-(2-(methylthio)-4-((3-nitrophenyl)amino)pyrimidin-5-yl)-3-oxopropanoate (5.90 g, 15.7 mmol) with DBU (2.35 mL) and N-ethyl-N,N-diisopropylethylamine (19.1.0 mL). The title compound was obtained as a light brown solid (4.50 g, 13.6 mmol, 87%).

(ES, m/z): [M+H]⁺=331.1.

¹H NMR (500 MHz, DMSO-d₆) δ 2.15 (3H, s), 5.88 (1H, s), 7.72-7.92 (2H, m), 8.34 (2H, dq, J=8.7, 2.1 Hz), 8.92 (1H, s), 12.29 (1H, s).

### 2-(Methylthio)-8-(3-nitrophenyl)-7-oxo-7,8-dihydropyrido[2,3-d]pyrimidin-5-yl trifluoromethanesulfonate

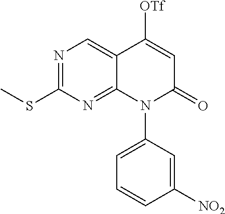 [SMILES: CSc1ncc2c(OS(=O)(=O)C(F)(F)F)cc(=O)n(-c3cccc([N+](=O)[O-])c3)c2n1]

General Procedure 8 was applied to 5-hydroxy-2-(methylthio)-8-(3-nitrophenyl)pyrido[2,3-d]pyrimidin-7(8H)-one (4.50 g, 13.6 mmol) with triethylamine (3.71.0 mL, 27.2 mmol) and triflic anhydride (3.43.0 mL, 20.4 mmol) in dichloromethane (150 mL). The crude product was purified by flash column chromatography eluting with ethyl acetate (0-100%) in 40-60 petroleum ether to yield the title compound as a beige solid (3.10 g, 6.70 mmol, 49%).

(ES, m/z): [M+H]⁺=463.1

¹H NMR (500 MHz, DMSO-d₆) δ 2.15 (3H, s), 5.88 (1H, s), 7.77-7.88 (2H, m), 8.29-8.37 (2H, m), 8.93 (1H, s).

### 2-(Methylthio)-8-(3-nitrophenyl)-5-((triisopropylsilyl)ethynyl)pyrido[2,3-d]pyrimidin-7(8H)-one

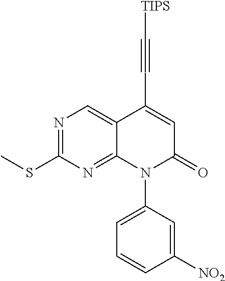

General Procedure 9 was applied to 2-(methylthio)-8-(3-nitrophenyl)-7-oxo-7,8-dihydropyrido[2,3-d]pyrimidin-5-yl trifluoromethanesulfonate (1.00 g, 2.16 mmol) with bis(triphenylphosphine)palladium(II) dichloride (151 mg, 0.216 mmol) and copper(I) iodide (41.0 mg, 0.216 mmol), N-ethyl-N,N-diisopropylethylamine (4.51.0 mL, 25.9 mmol), TIPS acetylene (0.967 mL, 4.32 mmol) and DMF (8.64 mL). The crude product was purified by flash column chromatography eluting with ethyl acetate (0-100%) in 40-60 petroleum ether to yield the title compound as a beige solid (1.03 g, 2.08 mmol, 96%).

(ES, m/z): [M+H]⁺=495.3

¹H NMR (500 MHz, DMSO-d₆) δ 0.98-1.24 (21H, m), 2.16 (3H, s), 6.95 (1H, s), 7.87 (2H, d, J=7.4 Hz), 8.38 (2H, q, J=2.4 Hz), 8.87 (1H, s).

### 2-((2-Methoxyphenyl)amino)-8-(3-nitrophenyl)-5-((triisopropylsilyl)ethynyl)pyrido[2,3-d]pyrimidin-7(8H)-one

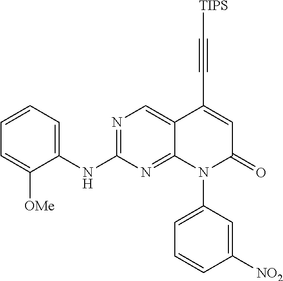

2-(Methylthio)-8-(3-nitrophenyl)-5-((triisopropylsilyl)ethynyl)pyrido[2,3-d]pyrimidin-7(8H)-one (3.10 g, 6.27 mmol, 1.0 eq.) and m-CPBA (4.62 g, 18.8 mmol, 3.0 eq.) were dissolved in dichloromethane (135 mL) and stirred at room temperature for 1 h. The reaction mixture was washed with saturated aqueous sodium thiosulfate solution (70 mL). The organic layer was separated, dried (Na₂SO₄) and concentrated under reduced pressure to yield a beige solid (2.70 g, 5.15 mmol, 82%). The resulting solid (2.70 g, 5.15 mmol, 1.0 eq.) and o-anisidine (0.581.0 mL, 5.15 mmol, 1.0 eq.) were dissolved in acetonitrile (16.0 mL) and stirred at 110° C. for 4 h. The solvent was removed under reduced pressure and the residue obtained was purified by flash column chromatography eluting with ethyl acetate (0-100%) in 40-60 petroleum ether to yield the title compound as a yellow solid (1.97 g, 3.46 mmol, 67%).

(ES, m/z): [M+H]⁺=570.4

¹H NMR (500 MHz, DMSO-d₆) δ 1.16 (18H, d, J=6.7 Hz), 1.19-1.29 (3H, m), 3.77 (3H, s), 6.68 (1H, s), 6.88-6.98 (2H, m), 7.55 (2H, t, J=8.1 Hz), 7.72 (2H, ddd, J=8.0, 2.2, 1.1 Hz), 8.33-8.44 (1H, m), 8.66 (1H, s), 8.77 (1H, s), 13.32 (1H, s).

### 5-Ethynyl-2-((2-methoxyphenyl)amino)-8-(3-nitrophenyl)pyrido[2,3-d]pyrimidin-7(8H)-one

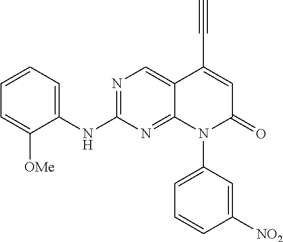 [SMILES: C#Cc1cc(=O)n(-c2cccc([N+](=O)[O-])c2)c2nc(Nc3ccccc3OC)ncc12]

General procedure 15 was applied to 2-((2-methoxyphenyl)amino)-8-(3-nitrophenyl)-5-((triisopropylsilyl)ethynyl)pyrido[2,3-d]pyrimidin-7(8H)-one (50.0 mg, 0.0878 mmol) and potassium fluoride (50.9 mg, 0.878 mmol) in DMF (1.67 mL). The crude product was purified by flash column chromatography eluting with ethyl acetate (0-100%) in 40-60 petroleum ether to afford the title compound as a yellow solid (8.00 mg, 0.0194 mmol, 22%).

(ES, m/z): [M+H]⁺=414.2.

¹H NMR (500 MHz, CDCl₃) b 3.67 (1H, s), 3.79 (3H, s), 6.26-6.40 (2H, m), 6.69 (1H, s), 6.75 (1H, d, J=8.1 Hz), 6.83 (1H, d, J=8.1 Hz), 7.62 (1H, d, J=8.0 Hz), 7.73 (1H, t, J=8.1 Hz), 8.01 (1H, s), 8.18 (1H, t, J=2.1 Hz), 8.38 (1H, d, J=8.3 Hz), 8.86 (1H, s).

### Example 55

### 8-(3-Aminophenyl)-2-((2-methoxyphenyl)amino)-5((triisopropylsilyl)ethynyl)pyrido[2,3-d]pyrimidin-7(8H)-one

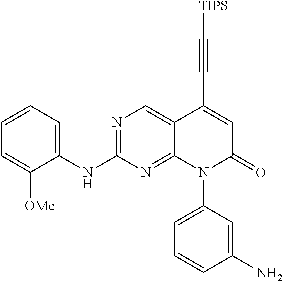

2-((2-Methoxyphenyl)amino)-8-(3-nitrophenyl)-5-((triisopropylsilyl)ethynyl)pyrido[2,3-d]pyrimidin-7(8H)-one (710 mg, 1.25 mmol, 1.0 eq.), iron (209 mg, 3.75 mmol, 3.0 eq.) and ammonium chloride (134 mg, 2.50 mmol, 2.0 eq.) were dissolved in a mixture of IPA/water (13:2.6 mL) and stirred at 80° C. for 4 h. The resulting solution was filtered through Celite, and the filtrate was concentrated under reduced pressure. The crude material was purified by flash column chromatography eluting with ethyl acetate (0-100%) in 40-60 petroleum ether to yield the title compound as a light brown solid (430 mg, 0.797 mmol, 64%).

(ES, m/z): [M+H]⁺=540.4.

¹H NMR (500 MHz, DMSO-d₆) δ 1.08-1.21 (21H, m), 3.82 (3H, s), 5.32 (2H, s), 6.41 (1H, d, J=7.7 Hz), 6.45 (1H, d, J=2.1 Hz), 6.71-6.75 (1H, m), 6.96 (1H, q, J=7.7 Hz), 7.19 (1H, t, J=7.9 Hz), 7.55 (1H, t, J=8.1 Hz), 7.64 (1H, d, J=8.2 Hz), 7.71 (1H, dd, J=8.6, 2.0 Hz), 7.84-7.95 (1H, m), 8.39 (1H, s), 8.75 (1H, s).

### 8-(3-Aminophenyl)-5-ethynyl-2-((2-methoxyphenyl)amino)pyrido[2,3-d]pyrimidin-7(8H)-one

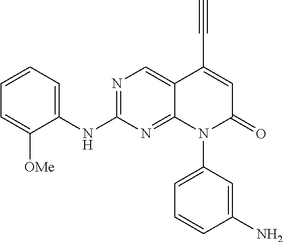 [SMILES: C#Cc1cc(=O)n(-c2cccc(N)c2)c2nc(Nc3ccccc3OC)ncc12]

General Procedure 15 was applied to 8-(3-aminophenyl)-2-((2-methoxyphenyl)amino)-5-((triisopropylsilyl)ethynyl)pyrido[2,3-d]pyrimidin-7(8H)-one (100 mg, 0.185 mmol) with potassium fluoride (107 mg, 1.85 mmol) in DMF (3.52.0 mL). The crude product was purified by flash column chromatography eluting with ethyl acetate (0-100%) in 40-60 petroleum ether to yield the title compound as a yellow solid (9.50 mg, 24.8 μmol, 13%).

(ES, m/z): [M+H]⁺=384.2

¹H NMR (500 MHz, CDCl₃) b 3.61 (1H, s), 3.75 (2H, s), 3.80 (3H, s), 6.50-6.58 (2H, m), 6.61 (1H, dd, J=7.7, 1.7 Hz), 6.68 (1H, s), 6.75 (1H, d, J=8.0 Hz), 6.79-6.88 (3H, m), 7.31 (1H, t, J=7.9 Hz), 8.03 (1H, s), 8.81 (1H, s).

### Example 56

### N-(3-(2-((2-methoxyphenyl)amino)-7-oxo-5-((triisopropylsilyl)ethynyl)pyrido[2,3-d]pyrimidin-8(7H)-yl)phenyl)acetamide

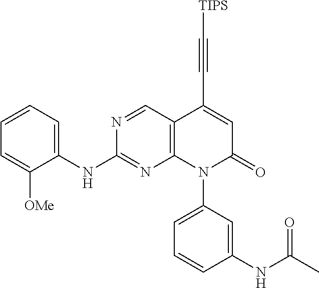

8-(3-Aminophenyl)-2-((2-methoxyphenyl)amino)-5((triisopropylsilyl)ethynyl)pyrido[2,3-d]pyrimidin-7(8H)-one (200 mg, 0.371 mol, 1.0 eq.) was dissolved in THF (10.0 mL) and acetic anhydride (0.500 mL, 5.29 mmol, 14 eq.) was added and the reaction was stirred at room temperature overnight. The reaction mixture was concentrated under reduced pressure and the crude material was purified by flash column chromatography eluting with ethyl acetate (0-100%) in 40-60 petroleum ether to yield as a yellow solid (110 mg, 0.189 mmol, 51%).

### N-(3-(5-Ethynyl-2-((2-methoxyphenyl)amino)-7-oxopyrido[2,3-d]pyrimidin-8(7H)-yl)phenyl)acetamide

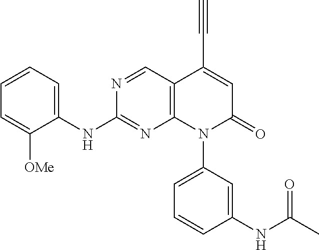 [SMILES: C#Cc1cc(=O)n(-c2cccc(NC(C)=O)c2)c2nc(Nc3ccccc3OC)ncc12]

General procedure 15 was applied to N-(3-(2-((2-methoxyphenyl)amino)-7-oxo-5-((triisopropylsilyl)ethynyl)pyrido[2,3-d]pyrimidin-8(7H)-yl)phenyl)acetamide (50.0 mg, 0.0859 mmol) with potassium fluoride (49.8 mg, 0.859 mmol) and DMF (1.63.0 mL). The crude product was purified by flash column chromatography eluting with ethyl acetate (0-100%) in 40-60 petroleum ether followed by trituration with 40-60 petroleum ether to yield the title compound as a yellow solid (4.90 mg, 11.5 μmol, 13%).

(ES, m/z): [M+H]⁺=426.3.

¹H NMR (500 MHz, DMSO-d₆) δ 2.04 (3H, s), 3.81 (3H, s), 5.15 (1H, s), 6.39-6.49 (1H, m), 6.72 (1H, s), 6.89-7.00 (2H, m), 7.02 (1H, d, J=7.8 Hz), 7.38-7.56 (2H, m), 7.61 (1H, s), 7.69 (1H, d, J=8.4 Hz), 8.45 (1H, s), 8.83 (1H, s), 10.17 (1H, s).

### Example 57

### N-(3-(2-((2-methoxyphenyl)amino)-7-oxo-5-((triisopropylsilyl)ethynyl)pyrido[2,3-d]pyrimidin-8(7H)-yl)phenyl)methanesulfonamide

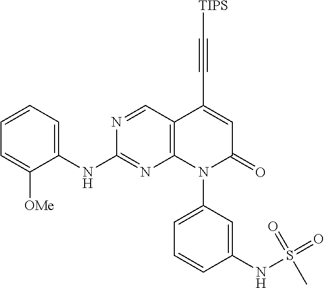

8-(3-Aminophenyl)-2-((2-methoxyphenyl)amino)-5((triisopropylsilyl)ethynyl)pyrido[2,3-d]pyrimidin-7(8H)-one (200 mg, 0.371 mmol, 1.0 eq.) was dissolved in dichloromethane (2.50 mL) and methanesulfonyl chloride (0.539 mL, 6.97 mmol, 18 eq.) was added and the reaction was stirred at 65° C. overnight. The reaction mixture was concentrated under reduced pressure and purified by flash column chromatography eluting with ethyl acetate (0-100%) in 40-60 petroleum ether to yield the title compound as a yellow solid (60.0 mg, 0.0971 mmol, 26%).

### N-(3-(5-Ethynyl-2-((2-methoxyphenyl)amino)-7-oxopyrido[2,3-d]pyrimidin-8(7H)-yl)phenyl)methanesulfonamide

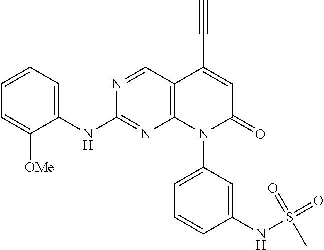 [SMILES: C#Cc1cc(=O)n(-c2cccc(NS(C)(=O)=O)c2)c2nc(Nc3ccccc3OC)ncc12]

General Procedure 15 was applied to N-(3-(2-((2-methoxyphenyl)amino)-7-oxo-5-((triisopropylsilyl)ethynyl)pyrido[2,3-d]pyrimidin-8(7H)-yl)phenyl)methanesulfonamide (50.0 mg, 80.9 μmol) with potassium fluoride (46.9 mg, 0.809 mmol) and DMF (1.54 mL). The crude product was purified by flash column chromatography eluting with ethyl acetate (0-100%) in 40-60 petroleum ether, followed by trituration with 40-60 petroleum ether to yield the title compound as a yellow solid (15.0 mg, 32.5 μmol, 41%).

(ES, m/z): [M+H]⁺=462.2.

¹H NMR (500 MHz, DMSO-d₆) δ 2.91 (3H, s), 3.81 (3H, s), 5.15 (1H, s), 6.53 (1H, s), 6.67-6.73 (1H, m), 6.90-7.02 (2H, m), 7.08-7.16 (2H, m), 7.39 (2H, dd, J=14.0, 8.2 Hz), 7.55 (1H, t, J=8.0 Hz), 8.48 (1H, s), 8.83 (1H, s), 10.03 (1H, s).

### Example 58

### N-(3-(2-((2-methoxyphenyl)amino)-7-oxo-5-((triisopropylsilyl)ethynyl)pyrido[2,3-d]pyrimidin-8(7H)-yl)phenyl)cyclopropanesulfonamide

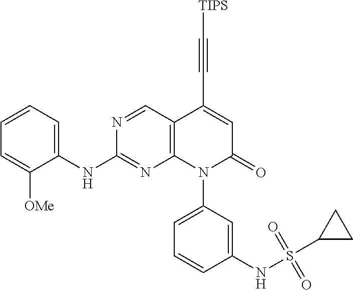

To a solution of 8-(3-Aminophenyl)-2-((2-methoxyphenyl)amino)-5((triisopropylsilyl)ethynyl)pyrido[2,3-d]pyrimidin-7(8H)-one (400 mg, 0.741 mmol, 1.0 eq.) and N-ethyl-N,N-diisopropylethylamine (0.150 mL, 0.863 mmol, 1.2 eq.) in dichloromethane (15.0 mL) was added cyclopropanesulfonyl chloride (100 μL, 0.980 mmol, 1.3 eq.) and the reaction mixture was stirred at 65° C. overnight. The solvent was removed under reduced pressure and the crude material was purified by flash column chromatography eluting with ethyl acetate (0-100%) in 40-60 petroleum to yield the title compound as a yellow solid (118 mg, 0.183 mmol, 25%).

### N-(3-(5-Ethynyl-2-((2-methoxyphenyl)amino)-7-oxopyrido[2,3-d]pyrimidin-8(7H)-yl)phenyl)cyclopropanesulfonamide

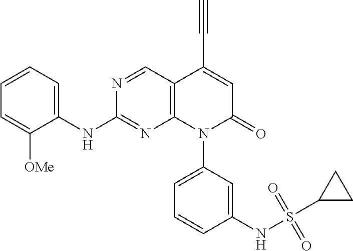 [SMILES: C#Cc1cc(=O)n(-c2cccc(NS(=O)(=O)C3CC3)c2)c2nc(Nc3ccccc3OC)ncc12]

General Procedure 15 was applied to N-(3-(2-((2-methoxyphenyl)amino)-7-oxo-5-((triisopropylsilyl)ethynyl)pyrido[2,3-d]pyrimidin-8(7H)-yl)phenyl)cyclopropanesulfonamide (50.0 mg, 77.7 μmol) with potassium fluoride (45.1 mg, 0.777 mmol) and DMF (1.48 mL). The crude product was purified by flash column chromatography eluting with ethyl acetate (0-100%) in 40-60 petroleum ether to yield the title compound as a yellow solid (9.30 mg, 19.1 μmol, 25%).

(ES, m/z): [M+H]⁺=488.3.

¹H NMR (500 MHz, DMSO-d₆) δ 0.80 (2H, d, J=8.9 Hz), 0.86-1.04 (2H, m), 3.81 (3H, s), 5.16 (1H, s), 6.54 (1H, s), 6.67-6.71 (1H, m), 6.90-6.98 (2H, m), 7.10 (1H, d, J=7.8 Hz), 7.16 (1H, t, J=2.1 Hz), 7.31-7.49 (2H, m), 7.53 (1H, t, J=8.0 Hz), 8.47 (1H, s), 8.83 (1H, s), 10.01 (1H, s), 1 CH signal under the DMSO signal.

### Example 59

### 2-((3-(2-((2-methoxyphenyl)amino)-7-oxo-5-((triisopropylsilyl)ethynyl)pyrido[2,3-d]pyrimidin-8(7H)-yl)phenyl)amino)-2-oxoethyl acetate

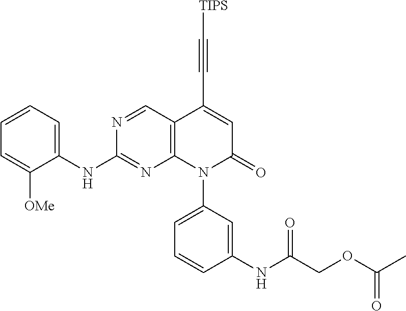

A solution of 8-(3-Aminophenyl)-2-((2-methoxyphenyl)amino)-5((triisopropylsilyl)ethynyl)pyrido[2,3-d]pyrimidin-7(8H)-one (200 mg, 0.371 mmol, 1.0 eq.) and acetoxy acetyl chloride (39.9 μL, 0.371 mmol, 1.0 eq.) in dichloromethane (2.50 mL) was stirred at room temperature for 30 min, triethylamine (25.9 μL, 0.186 mmol, 0.5 eq.) was added and the reaction was further stirred for 1 h. The solvent was removed under reduced pressure and the crude material was purified by flash column chromatography eluting with ethyl acetate (0-100%) in 40-60 petroleum ether to yield as a yellow solid (45.0 mg, 70.3 μmol, 19%).

### 2-((3-(5-Ethynyl-2-((2-methoxyphenyl)amino)-7-oxopyrido[2,3-d]pyrimidin-8(7H)-yl)phenyl)amino)-2-oxoethyl acetate

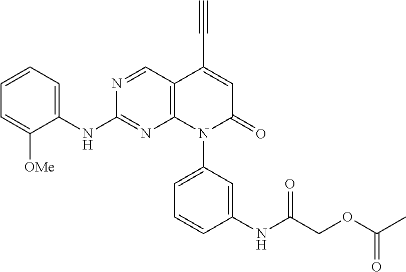 [SMILES: C#Cc1cc(=O)n(-c2cccc(NC(=O)COC(C)=O)c2)c2nc(Nc3ccccc3OC)ncc12]

General Procedure 15 was applied to 2-((3-(2-((2-methoxyphenyl)amino)-7-oxo-5-((triisopropylsilyl)ethynyl)pyrido[2,3-d]pyrimidin-8(7H)-yl)phenyl)amino)-2-oxoethyl acetate (45.0 mg, 70.3 μmol) with potassium fluoride (40.8 mg, 0.703 mmol) and DMF (1.34 mL). The crude product was purified by flash column chromatography eluting with ethyl acetate (0-100%) in 40-60 petroleum ether to yield the title compound as a yellow solid (11.0 mg, 0.0228 mmol, 32%).

(ES, m/z): [M+H]⁺=484.3.

¹H NMR (500 MHz, DMSO-d₆) δ 2.10 (3H, d, J=1.0 Hz), 3.81 (3H, s), 4.65 (2H, s), 5.15 (1H, s), 6.51 (1H, s), 6.67-6.71 (1H, m), 6.94 (2H, d, J=11.0 Hz), 7.01-7.14 (1H, m), 7.44 (1H, d, J=8.1 Hz), 7.52 (1H, t, J=8.0 Hz), 7.59 (1H, s), 7.69-7.79 (1H, m), 8.47 (1H, s), 8.82 (1H, s), 10.31 (1H, s).

### Example 60

### Ethyl 2-(methylthio)-4-((4-nitrophenyl)amino)pyrimidine-5-carboxylate

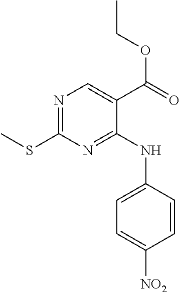 [SMILES: CCOC(=O)c1cnc(SC)nc1Nc1ccc([N+](=O)[O-])cc1]

Ethyl 4-chloro-2-(methylthio)pyrimidine-5-carboxylate (30.0 g, 129 mmol, 1.0 eq.), 4-nitroaniline (44.6 g, 323 mmol, 2.5 eq.) and triethylamine (71.9 mL, 516 mmol, 4.0 eq.) were dissolved in THF (300 mL) and stirred at 85° C. for 72 h. The resulting precipitate was collected by vacuum filtration and washed with THF (500 mL) to yield the title compound as an off-white solid (26.6 g, 79.6 mmol, 62%).

(ES, m/z): [M+H]⁺=335.1.

¹H NMR (500 MHz, CDCl₃) δ 1.35 (3H, t, J=7.3 Hz), 2.56 (3H, s), 4.37 (2H, q, J=7.2 Hz), 7.82-7.87 (2H, m), 8.15-8.21 (2H, m), 8.81 (1H, s), 10.89 (1H, s).

### 2-(Methylthio)-4-((4-nitrophenyl)amino)pyrimidine-5-carboxylic acid

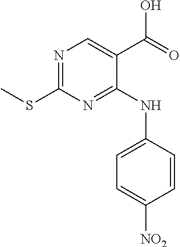 [SMILES: CSc1ncc(C(=O)O)c(Nc2ccc([N+](=O)[O-])cc2)n1]

To a solution of ethyl 2-(methylthio)-4-((4-nitrophenyl)amino)pyrimidine-5-carboxylate (13.0 g, 38.9 mmol, 1.0 eq.) in a mixture of THF/methanol/water (1:1:1) (192.0 mL) was added lithium hydroxide monohydrate (5.68 g, 150 mmol, 4.0 eq.) and the reaction mixture was stirred at room temperature overnight. The solvent was removed under reduced pressure, and the resulting aqueous residue was acidified to pH 4-5 using a 1 N hydrochloric acid solution. The resulting solid was collected by vacuum filtration, washed with water and dried in a vacuum oven (at 40° C.) to give the title compound as an off-white solid (7.60 g, 24.8 mmol, 64%).

(ES, m/z): [M+H]⁺=307.1.

¹H NMR (500 MHz, DMSO-d₆) δ 2.56 (1H), 7.99 (2H, d, J=9.0 Hz), 8.26 (2H, d, J=9.1 Hz), 8.78 (1H, s), 11.11 (1H, s).

### 2-(Methylthio)-4-((4-nitrophenyl)amino)pyrimidine-5-carbonyl chloride

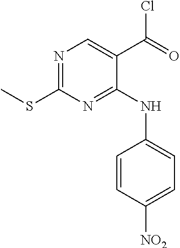 [SMILES: CSc1ncc(C(=O)Cl)c(Nc2ccc([N+](=O)[O-])cc2)n1]

To a solution of 2-(methylthio)-4-((4-nitrophenyl)amino)pyrimidine-5-carboxylic acid (7.60 g, 24.8 mmol) in toluene (10.0 mL) was added thionyl chloride (30.0 mL) and the reaction stirred at 95° C. for 5 h. The solvent was evaporated to yield the title compound as a white solid (6.64 g, 20.4 mmol, 82%), which was used without further purification.

### Ethyl 3-(2-(methylthio)-4-((4-nitrophenyl)amino)pyrimidin-5-yl)-3-oxopropanoate

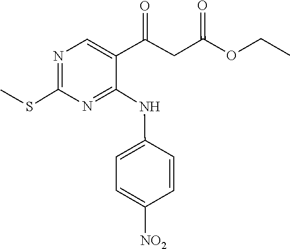 [SMILES: CCOC(=O)CC(=O)c1cnc(SC)nc1Nc1ccc([N+](=O)[O-])cc1]

General Procedure 5 was applied to 2-(methylthio)-4-((4-nitrophenyl)amino)pyrimidine-5-carbonyl chloride (3.18 g, 9.79 mmol) in THF (9.79 mL) with 1 M LiHMDS in THF (34.3.0 mL, 34.3 mmol), EtOAc (1.91.0 mL, 19.6 mmol) and THF (29.4 mL) at 0° C. The title compound was obtained as a light-yellow solid (2.80 g, 7.44 mmol, 76%).

(ES, m/z): [M+H]⁺=377.2.

¹H NMR (500 MHz, CDCl₃) δ 1.24 (3H, t, J=7.1 Hz), 2.55 (1H, s), 3.92 (2H, s), 4.19 (2H, q, J=7.2 Hz), 7.77-7.90 (2H, m), 8.13-8.24 (2H, m), 8.70 (1H, s), 10.81 (1H, s)

### 5-Hydroxy-2-(methylthio)-8-(4-nitrophenyl)pyrido[2,3-d]pyrimidin-7(8H)-one

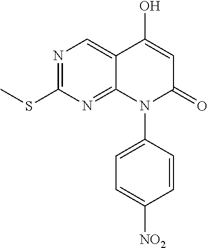 [SMILES: CSc1ncc2c(O)cc(=O)n(-c3ccc([N+](=O)[O-])cc3)c2n1]

General Procedure 6 was applied to ethyl 3-(2-(methylthio)-4-((4-nitrophenyl)amino)pyrimidin-5-yl)-3-oxopropanoate (2.80 g, 7.44 mmol) with DBU (1.11.0 mL, 7.42 mmol) and N,N-diisopropylethylamine (9.05 mL, 52.0 mmol). The title compound was obtained as a light brown solid (2.37 g, 7.17 mmol, 96%).

(ES, m/z): [M+H]⁺=331.1

¹H NMR (500 MHz, DMSO-d₆) δ 2.18 (3H, s), 5.88 (1H, s), 7.63-7.69 (2H, m), 8.36-8.42 (2H, m), 8.93 (1H, s), 12.32 (1H, s)

### 2-(Methylthio)-8-(4-nitrophenyl)-7-oxo-7,8-dihydropyrido[2,3-d]pyrimidin-5-yl trifluoromethanesulfonate

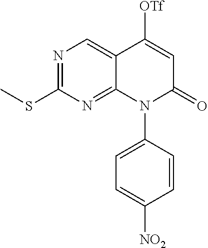 [SMILES: CSc1ncc2c(OS(=O)(=O)C(F)(F)F)cc(=O)n(-c3ccc([N+](=O)[O-])cc3)c2n1]

General Procedure 7 was applied to 5-hydroxy-2-(methylthio)-8-(4-nitrophenyl)pyrido[2,3-d]pyrimidin-7(8H)-one (2.37 g, 7.17 mmol) with triethylamine (1.99 mL, 14.3 mmol), triflic anhydride (1.81.0 mL, 10.8 mmol) and dichloromethane (78.9 mL). The crude product was purified by flash column chromatography eluting with ethyl acetate (0-50%) in 40-60 petroleum ether to yield the title compound as a beige solid (1.36 g, 2.94 mmol, 41%).

(ES, m/z): [M+H]⁺=463.1

¹H NMR (500 MHz, CDCl₃) δ 2.16 (3H, s), 6.67 (1H, s), 7.38-7.44 (2H, m), 8.31-8.41 (2H, m), 8.78 (1H, s).

### 2-(Methylthio)-8-(4-nitrophenyl)-5-((triisopropylsilyl)ethynyl)pyrido[2,3-d]pyrimidin-7(8H)-one

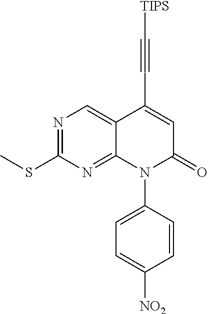

General Procedure 8 was applied to 2-(methylthio)-8-(4-nitrophenyl)-7-oxo-7,8-dihydropyrido[2,3-d]pyrimidin-5-yl trifluoromethanesulfonate (1.00 g, 2.16 mmol) with bis(triphenylphosphine)palladium(II) dichloride (151 mg, 0.216 mmol), copper(I) iodide (41.0 mg, 0.216 mmol), DMF (8.64 mL), N,N-diisopropylethylamine (4.51.0 mL, 25.9 mmol) and TIPS acetylene (0.967 mL, 4.32 mmol). The crude product was purified by flash column chromatography eluting with ethyl acetate (0-100%) in 40-60 petroleum ether to yield the title compound as a beige solid (850 mg, 1.72 mmol, 80%).

(ES, m/z): [M+H]⁺=495.3

¹H NMR (500 MHz, CDCl₃) δ 0.85-1.25 (21H, m), 2.13 (3H, s), 6.78 (1H, s), 7.35-7.45 (2H, m), 8.30-8.38 (2H, m), 8.95 (1H, s).

### 2-((2-Methoxyphenyl)amino)-8-(4-nitrophenyl)-5((triisopropylsilyl)ethynyl) pyrido[2,3-d]pyrimidin-7(8H)-one

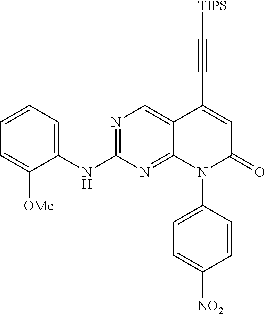

2-(Methylthio)-8-(4-nitrophenyl)-5-((triisopropylsilyl)ethynyl)pyrido[2,3-d]pyrimidin-7(8H)-one (1.10 g, 2.22 mmol, 1.0 eq.) and mCPBA (1.64 g, 6.66 mmol, 3.0 eq.) were dissolved in dichloromethane (47.0 mL) and stirred at room temperature for 1 hr. The reaction mixture was washed with saturated aqueous sodium thiosulfate solution (30 mL). The organic layer was separated, dried (Na₂SO₄) and concentrated under reduced pressure to afford a beige solid (1.73 g). This beige solid (1.73 g, 3.30 mmol, 1.0 eq.) and o-anisidine (0.372.0 mL, 3.30 mmol, 1.0 eq.) were dissolved in acetonitrile (10.0 mL) and stirred at 110° C. for 6 h. The solvent was removed under reduced pressure and the crude material was purified by flash column chromatography eluting with ethyl acetate (0-100%) in 40-60 petroleum ether to yield the title compound as a brown solid (1.06 g, 1.86 mmol, 56%).

(ES, m/z): [M+H]⁺=570.4.

¹H NMR (500 MHz, CDCl₃) δ 1.04-1.18 (21H, m), 3.79 (3H, s), 6.68 (1H, s), 7.36-7.38 (2H, m), 7.42-7.46 (2H, m), 7.52 (1H, ddd, J=8.0, 2.2 Hz), 7.93 (1H, dt, J=7.8, 1.3 Hz), 8.38 (2H, d, J=7.0 Hz), 8.84 (1H, s). NH not visualised.

### 5-Ethynyl-2-((2-methoxyphenyl)amino)-8-(4-nitrophenyl)pyrido[2,3-d]pyrimidin-7(8H)-one

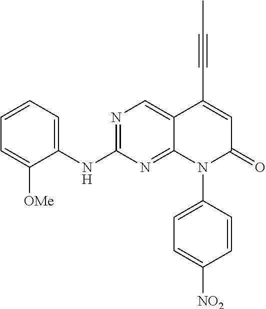 [SMILES: CC#Cc1cc(=O)n(-c2ccc([N+](=O)[O-])cc2)c2nc(Nc3ccccc3OC)ncc12]

General Procedure 15 was applied to 2-((2-methoxyphenyl)amino)-8-(4-nitrophenyl)-5-((triisopropylsilyl)ethynyl)pyrido[2,3-d]pyrimidin-7(8H)-one (70.0 mg, 0.123 mmol) with potassium fluoride (71.3 mg, 1.23 mmol) and DMF (2.34 mL). The crude product was purified by flash column chromatography eluting with ethyl acetate (0-100%) in 40-60 petroleum ether to yield the title compound as a yellow solid (11.0 mg, 26.6 mmol, 22%).

(ES, m/z): [M+H]⁺=414.2

¹H NMR (500 MHz, DMSO-d₆) δ 3.78 (3H, s), 5.17 (1H, s), 6.73 (1H, s), 6.84-6.94 (3H, m), 7.27 (1H, m), 7.71 (2H, d, J=8.9 Hz), 8.43 (2H, d, J=8.9 Hz), 8.66 (1H, s), 8.84 (1H, s).

### Example 61

### 8-(4-Aminophenyl)-2-((2-methoxyphenyl)amino)5((triisopropylsilyl)ethynyl)pyrido[2,3-d]pyrimidin-7(8H)-one

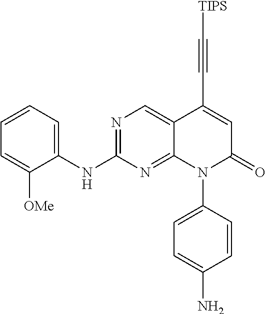

2-((2-Methoxyphenyl)amino)-8-(4-nitrophenyl)-5-((triisopropylsilyl)ethynyl)pyrido[2,3-d]pyrimidin-7(8H)-one (2.40 g, 4.21 mmol, 1.0 eq.), iron (704 mg, 12.6 mmol, 3.0 eq.) and ammonium chloride (450 mg, 8.42 mmol, 2.0 eq.) were dissolved in IPA (42.0 mL) and water (8.40 mL) and the reaction mixture was stirred at 80° C. for 4 h. The resulting solution was filtered through Celite and the filtrate was concentrated under reduced pressure. The crude material was purified by flash column chromatography eluting with ethyl acetate (0-100%) in 40-60 petroleum ether to yield the title compound as a yellow-orange solid (1.67 g, 3.09 mmol, 73%).

(ES, m/z): [M+H]⁺=540.4

¹H NMR (500 MHz, DMSO-d₆) 51.05-1.24 (21H, m), 3.82 (3H, s), 6.62 (1H, s), 6.72 (2H, d, J=8.4 Hz), 6.91 (2H, d, J=8.4 Hz), 6.94 (2H, s), 7.56 (1H, d, J=8.1 Hz), 7.71 (2H, dt, J=8.1, 1.3 Hz), 7.87-7.95 (1H, m), 8.35 (1H, s), 8.74 (1H, s).

### 8-(4-Aminophenyl)-5-ethynyl-2-((2-methoxyphenyl)amino)pyrido[2,3-d]pyrimidin-7(8H)-one

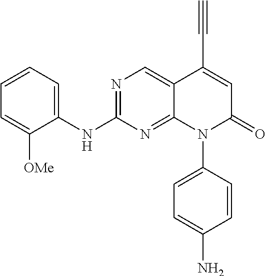 [SMILES: C#Cc1cc(=O)n(-c2ccc(N)cc2)c2nc(Nc3ccccc3OC)ncc12]

General Procedure 15 was applied to 8-(3-aminophenyl)-2-((2-methoxyphenyl)amino)-5-((triisopropylsilyl)ethynyl)pyrido[2,3-d]pyrimidin-7(8H)-one (80.0 mg, 0.148 mmol) with potassium fluoride (85.8 mg, 1.48 mmol) and DMF (2.81.0 mL). The crude product was purified by flash column chromatography eluting with ethyl acetate (0-100%) in 40-60 petroleum ether to yield the title compound as a yellow solid (8.50 mg, 22.2 μmol, 15%).

(ES, m/z): [M+H]⁺=384.2.

¹H NMR (500 MHz, DMSO-d₆) δ 3.83 (3H, s), 5.11 (1H, s), 5.38 (2H, s), 6.67-6.74 (4H, m), 6.88-7.04 (4H, m), 7.62-7.71 (1H, m), 8.33 (1H, s), 8.79 (1H, s).

### Example 62

### (N-(4-(2-((2-methoxyphenyl)amino)-7-oxo-5-((triisopropylsilyl)ethynyl)pyrido[2,3-d]pyrimidin-8(7H)-yl)phenyl)acetamide

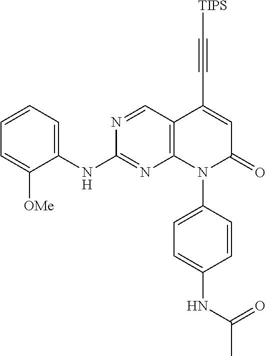

A solution of 8-(4-aminophenyl)-2-((2-methoxyphenyl)amino)-5((triisopropylsilyl)ethynyl)pyrido[2,3-d]pyrimidin-7(8H)-one (200 mg, 0.371 mmol) and acetic anhydride (0.50 mL, 5.29 mmol) in THF (10.0 mL) was stirred at room temperature for 30 min, triethylamine (25.9 μL, 0.186 mmol) was added and the reaction mixture was stirred for 1 h. The solvents were removed under reduced pressure and the crude material was purified by flash column chromatography eluting with ethyl acetate (0-100%) in 40-60 petroleum ether to yield the title compound as a yellow solid (125 mg, 0.215 mmol, 58%).

### N-(4-(5-Ethynyl-2-((2-methoxyphenyl)amino)-7-oxopyrido[2,3-d]pyrimidin-8(7H)-yl)phenyl)acetamide

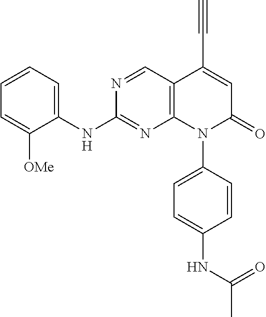 [SMILES: C#Cc1cc(=O)n(-c2ccc(NC(C)=O)cc2)c2nc(Nc3ccccc3OC)ncc12]

General procedure 15 was applied to (N-(4-(2-((2-methoxyphenyl)amino)-7-oxo-5-((triisopropylsilyl)ethynyl)pyrido[2,3-d]pyrimidin-8(7H)-yl)phenyl)acetamide (50.0 mg, 0.0859 mmol) with potassium fluoride (49.8 mg, 0.859 mmol) and DMF (1.63.0 mL). The crude material was purified by flash column chromatography eluting with ethyl acetate (0-100%) in 40-60 petroleum ether to yield the title compound as a yellow solid (9.30 mg, 0.0219 mmol, 25%).

(ES, m/z): [M+H]⁺=426.2

¹H NMR (500 MHz, DMSO-d₆) δ 2.14 (3H, s), 3.82 (3H, s), 5.14 (1H, s), 6.45-6.50 (1H, m), 6.71 (1H, s), 6.87-7.03 (2H, m), 7.17-7.29 (2H, m), 7.47 (1H, d, J=8.0 Hz), 7.65-7.81 (2H, m), 8.41 (1H, s), 8.82 (1H, s), 10.20 (1H, s).

### Example 63

### N-(4-(2-((2-methoxyphenyl)amino)-7-oxo-5-((triisopropylsilyl)ethynyl)pyrido[2,3-d]pyrimidin-8(7H)-yl)phenyl)methanesulfonamide

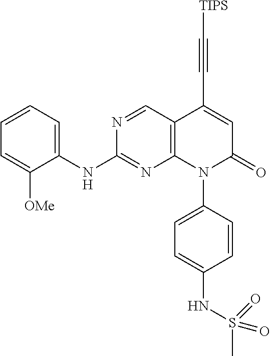

To a solution of 8-(4-aminophenyl)-2-((2-methoxyphenyl)amino)-5((triisopropylsilyl)ethynyl)pyrido[2,3-d]pyrimidin-7(8H)-one (240 mg, 0.45 mmol, 1.0 eq.), triethylamine (0.215 mL, 1.54 mmol, 3.5 eq.) in dichloromethane (3.00 mL) was added methanesulfonyl chloride (37.9 μL, 0.490 mmol, 1.1 eq.) and the reaction mixture was stirred at room temperature for 1 h. The solvent was removed under reduced pressure and the crude material was purified by flash column chromatography eluting with ethyl acetate (0-100%) in 40-60 petroleum ether to yield as a yellow solid (30.0 mg, 48.6 μmol, 11%).

### N-(4-(5-Ethynyl-2-((2-methoxyphenyl)amino)-7-oxopyrido[2,3-d]pyrimidin-7(8H)-yl)phenyl)methanesulfonamide

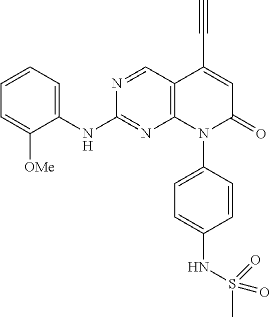 [SMILES: C#Cc1cc(=O)n(-c2ccc(NS(C)(=O)=O)cc2)c2nc(Nc3ccccc3OC)ncc12]

General procedure 15 was applied to N-(4-(2-((2-methoxyphenyl)amino)-7-oxo-5-((triisopropylsilyl)ethynyl)pyrido[2,3-d]pyrimidin-8(7H)-yl)phenyl)methanesulfonamide (30.0 mg, 48.6 μmol) with potassium fluoride (28.2 mg, 0.486 mmol) and DMF (0.923.0 mL).

The crude product was purified by flash column chromatography eluting with ethyl acetate (0-100%) in 40-60 petroleum ether to yield the title compound as a yellow solid (9.50 mg, 20.6 μmol, 42%).

(ES, m/z): [M+H]⁺=462.2.

¹H NMR (500 MHz, DMSO-d₆) δ 3.09 (3H, s), 3.81 (3H, s), 5.13 (1H, s), 6.61 (1H, s), 6.67-6.71 (1H, m), 6.96 (2H, d, J=3.9 Hz), 7.30 (2H, d, J=8.5 Hz), 7.37 (2H, d, J=8.5 Hz), 7.46 (1H, d, J=7.9 Hz), 8.47 (1H, s), 8.82 (1H, s). NH not visualised.

### Example 64

### 2-((4-(2-((2-methoxyphenyl)amino)-7-oxo-5-((triisopropylsilyl)ethynyl)pyrido[2,3-d]pyrimidin-8(7H)-yl)phenyl)amino)-2-oxoethyl acetate

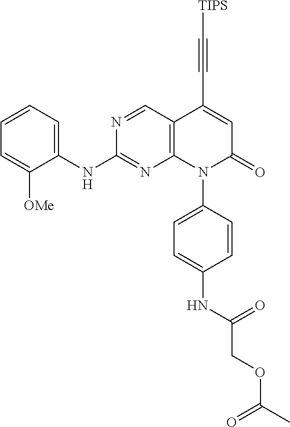

A solution of 8-(3-aminophenyl)-2-((2-methoxyphenyl)amino)-5((triisopropylsilyl)ethynyl)pyrido [2,3-d]pyrimidin-7(8H)-one (200 mg, 0.371 mmol, 1.0 eq.) and acetoxy acetyl chloride (39.9 μL, 0.371 mmol, 1.0 eq.) in dichloromethane (2.5 mL) was stirred at room temperature for 30 minutes, triethylamine (25.9 μL, 0.186 mmol, 0.5 eq.) was added and the reaction stirred for 1 h. The solvent was removed and the crude material was purified by flash column chromatography eluting with ethyl acetate (0-100%) in 40-60 petroleum ether to yield the title compound as a yellow solid (43.0 mg, 67.2 μmol, 18%).

### 2-((4-(5-Ethynyl-2-((2-methoxyphenyl)amino)-7-oxopyrido[2,3-d]pyrimidin-8(7H)-yl)phenyl)amino)-2-oxoethyl acetate

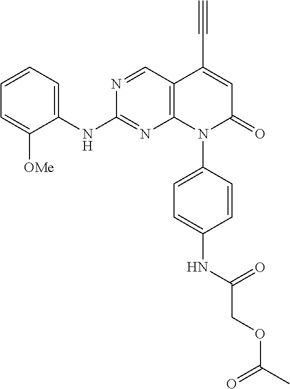 [SMILES: C#Cc1cc(=O)n(-c2ccc(NC(=O)COC(C)=O)cc2)c2nc(Nc3ccccc3OC)ncc12]

General procedure 15 was applied to 2-((4-(2-((2-methoxyphenyl)amino)-7-oxo-5-((triisopropylsilyl)ethynyl)pyrido[2,3-d]pyrimidin-8(7H)-yl)phenyl)amino)-2-oxoethyl acetate (43.0 mg, 67.2 μmol) with potassium fluoride (39.0 mg, 0.672 mmol) and DMF (1.28 mL). The crude product was purified by flash column chromatography eluting with ethyl acetate (0-100%) in 40-60 petroleum ether to yield the title compound as a yellow solid (13.0 mg, 26.9 μmol, 40%).

(ES, m/z): [M+H]⁺=484.3.

¹H NMR (400 MHz, DMSO-d₆) δ 2.17 (3H, s), 3.82 (3H, s), 4.75 (2H, s), 5.13 (1H, s), 6.51 (1H, s), 6.67-6.71 (1H, m), 6.92-7.09 (2H, m), 7.29 (2H, d, J=8.6 Hz), 7.44 (1H, d, J=8.2 Hz), 7.76 (2H, d, J=8.5 Hz), 8.41 (1H, s), 8.82 (1H, s), 10.33 (1H, s).

### Example 65

### N-(4-(2-((2-methoxyphenyl)amino)-7-oxo-5-((triisopropylsilyl)ethynyl)pyrido[2,3-d]pyrimidin-8(7H)-yl)phenyl)propionamide

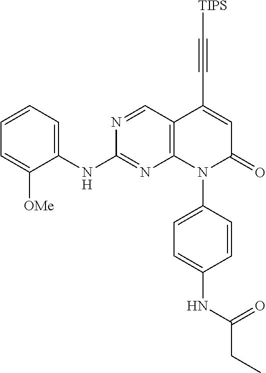

A solution of 8-(4-aminophenyl)-2-((2-methoxyphenyl)amino)-5((triisopropylsilyl)ethynyl)pyrido[2,3-d]pyrimidin-7(8H)-one (200 mg, 0.371 mmol, 1.0 eq.) and propionyl chloride (35.6 μL, 0.410 mmol, 1.1 eq.) in dichloromethane (2.4 mL) was stirred at room temperature for 30 minutes, triethylamine (25.9 μL, 0.186 mmol, 0.5 eq.) was added and the reaction mixture was stirred for 1 h. The solvent was removed under reduced pressure and the crude material was purified by flash column chromatography eluting with ethyl acetate (0-100%) in 40-60 petroleum ether to yield as a yellow solid (55.0 mg, 92.3 μmol, 25%).

### N-(4-(5-Ethynyl-2-((2-methoxyphenyl)amino)-7-oxopyrido[2,3-d]pyrimidin-7(8H)-yl)phenyl)propionamide

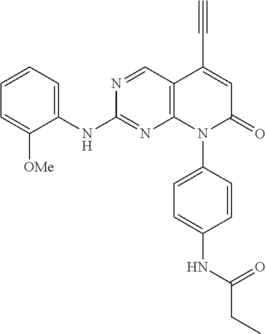 [SMILES: C#Cc1cc(=O)n(-c2ccc(NC(=O)CC)cc2)c2nc(Nc3ccccc3OC)ncc12]

General procedure 15 was applied to N-(4-(2-((2-methoxyphenyl)amino)-7-oxo-5-((triisopropylsilyl)ethynyl)pyrido[2,3-d]pyrimidin-8(7H)-yl)phenyl)propionamide (55.0 mg, 92.3 μmol) with potassium fluoride (53.5 mg, 0.923 mmol) and DMF (1.75 mL). The crude product was purified by flash column chromatography eluting with ethyl acetate (0-100%) in 40-60 petroleum ether to yield the title compound as a yellow solid (11.5 mg, 26.2 μmol, 28%).

(ES, m/z): [M+H]⁺=440.3.

¹H NMR (400 MHz, DMSO-d₆) δ 1.16 (3H, t, J=7.5 Hz), 2.42 (2H, q, J=7.6 Hz), 3.82 (3H, s), 5.13 (1H, s), 6.50 (1H, s), 6.65-6.70 (1H, m), 6.83-6.98 (2H, m), 7.26 (2H, d, J=8.7 Hz), 7.77 (2H, d, J=8.6 Hz), 7.47 (1H, d, J=8.0 Hz), 8.40 (1H, s), 8.82 (1H, s), 10.10 (1H, s).

### Example 66

### N-(4-(2-((2-methoxyphenyl)amino)-7-oxo-5-((triisopropylsilyl)ethynyl)pyrido[2,3-d]pyrimidin-8(7H)-yl)phenyl)cyclopropane carboxamide

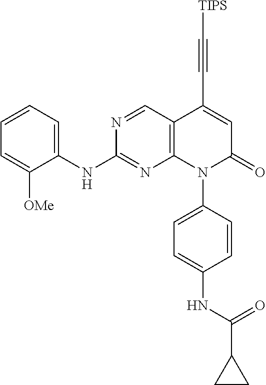

A solution 8-(4-Aminophenyl)-2-((2-methoxyphenyl)amino)-5((triisopropylsilyl)ethynyl)pyrido[2,3-d]pyrimidin-7(8H)-one (180 mg, 0.333 mmol, 1.0 eq.), triethylamine (0.162.0 mL, 1.16 mmol, 3.5 eq.) and cyclopropanecarbonyl chloride (32.7 μL, 0.360 mmol, 1.1 eq.) in dichloromethane (2.20 mL) was stirred at room temperature for 3 h. The solvent was removed under reduced pressure and the crude material was purified by flash column chromatography eluting with ethyl acetate (0-100%) in 40-60 petroleum ether to yield the title compound as a yellow solid (30.0 mg, 49.4 μmol, 15%).

### N-(4-(5-Ethynyl-2-((2-methoxyphenyl)amino)-7-oxopyrido[2,3-d]pyrimidin-8(7H)-yl)phenyl)cyclopropanecarboxamide

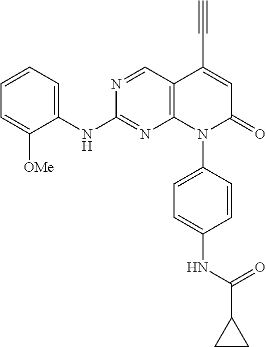 [SMILES: C#Cc1cc(=O)n(-c2ccc(NC(=O)C3CC3)cc2)c2nc(Nc3ccccc3OC)ncc12]

General procedure 15 was applied to N-(4-(2-((2-methoxyphenyl)amino)-7-oxo-5-((triisopropylsilyl)ethynyl)pyrido[2,3-d]pyrimidin-8(7H)-yl)phenyl)cyclopropane carboxamide (30.0 mg, 49.4 μmol) with potassium fluoride (28.7 mg, 0.494 mmol) and DMF (0.939 mL). The crude product was purified by flash column chromatography eluting with ethyl acetate (0-100%) in 40-60 petroleum ether to yield the title compound as a yellow solid (7.40 mg, 16.4 μmol, 33%).

(ES, m/z): [M+H]⁺=452.3.

¹H NMR (500 MHz, DMSO-d₆) δ 0.73-0.97 (4H, m), 1.83-1.89 (1H, m), 3.82 (3H, s), 5.11 (1H, s), 6.45-6.54 (1H, m), 6.70 (1H, s), 6.81-7.04 (2H, m), 7.19-7.31 (2H, m), 7.47 (1H, d, J=8.1 Hz), 7.76 (2H, d, J=8.7 Hz), 8.40 (1H, s), 8.82 (1H, s), 10.43 (1H, s).

### Example 67

### Ethyl 4-acetamido-2-(methylthio)pyrimidine-5-carboxylate

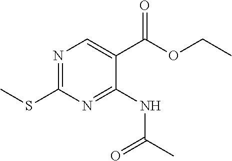 [SMILES: CCOC(=O)c1cnc(SC)nc1NC(C)=O]

A solution of ethyl 4-amino-2-(methylthiol)pyrimdine-5-carboxylate (2.00 g, 9.38 mmol, 1.0 eq.) in acetic anhydride (15 mL) was heated 150° C. under microwave irradiation for 20 minutes followed by 30 minutes. The solvent was removed under reduced pressure and the residue was dissolved in dichloromethane and filtered through a SCX column. The filtrate was concentrated under reduced pressure to yield the title compound (2.22 g, 8.67 mmol, 92%) which was used without further purification.

(ES, m/z): [M+H]⁺=256.1.

### 5-Hydroxy-2-(methylthio)pyrido[2,3-d]pyrimidin-7(8H)-one

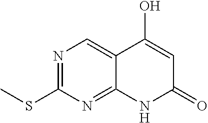

To a stirred solution of ethyl 4-acetamido-2-(methylthio)pyrimidine-5-carboxylate (1.00 g, 3.92 mmol, 1.0 eq.) in THF (39 mL, 0.1 M) at 0° C. was added 1.0 M KHMDS in THF (11.7 mL, 11.7 mmol, 3.0 eq.) dropwise. The reaction mixture was stirred at 0° C. for 30 minutes, then quenched with methanol and concentrated under reduced pressure. The crude material was suspended in water and acidified with 6 M aqueous hydrochloric acid. The resulting precipitate was collected by vacuum filtration and dried in vacuum oven (40° C.) overnight, to yield the title compound (568 mg, 2.71 mmol, 69%) which was used without any further purification.

(ES, m/z): [M+H]⁺=210.0.

### 2-(Methylthio)-5-((triisopropylsilyl)ethynyl)pyrido[2,3-d]pyrimidin-7(8H)-one

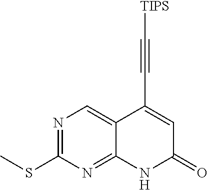

A solution of 5-hydroxy-2-(methylthio)pyrido[2,3-d]pyrimidin-7(8H)-one (6.00 g, 28.6 mmol, 1.0 eq.) and N,N-phenyl bis trifluoromethanesulfonimide (12.3 g, 36.4 mmol, 1.1 eq.) in THF (114 mL, 0.25 M) was sparged with nitrogen and triethylamine (8.00 mL, 57.4 mmol, 2.0 eq.) was added. The reaction mixture was stirred at 60° C. for 90 minutes, copper(I) iodide (546 mg, 2.86 mmol, 0.1 eq.), bis(triphenylphosphine)palladium(II) dichloride (2.10 g, 2.86 mmol, 0.1 eq.) and TIPS-acetylene (12.9 mL, 57.4 mmol, 2.0 eq.) were added and stirring was continued at 60° C. overnight. The reaction mixture was concentrated under reduced pressure, dissolved in dichloromethane (100 mL) and washed with water (100 mL). The aqueous layer was extracted with dichloromethane (5×50 mL) and the combined organic layers were dried (MgSO₄) and concentrated under reduced pressure. The crude material was purified by flash column chromatography eluting with ethyl acetate (10-30%) in 40-60 petroleum ether to yield the title compound as a pale yellow solid (3.38 g, 9.06 mmol, 32%).

(ES, m/z): [M+H]⁺=374.4.

### 8-(Cyclopentylmethyl)-2-(methylthio)-5-((triisopropylsilyl)ethynyl)pyrido[2,3-d]pyrimidin-7(8H)-one

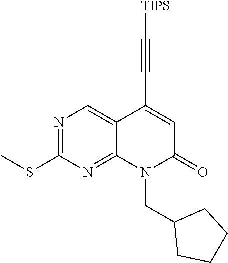

General procedure 18 was applied to 2-(methylthio)-5-((triisopropylsilyl)ethynyl)pyrido[2,3-d]pyrimidin-7(8H)-one (500 mg, 1.34 mmol) with cyclopentylmethanol (170 μL, 1.61 mmol), triphenylphosphine (527 g, 2.01 mmol) and DIAD (320 μL, 2.01 mmol) in dichloromethane (14 mL). The crude material was purified by flash column chromatography eluting with ethyl acetate (0-20%) in 40-60 petroleum ether to yield the title compound as a colourless oil (295 mg, 0.648 mmol, 48%).

(ES, m/z): [M+H]⁺=456.5.

### 8-(Cyclopentylmethyl)-2-(methylsulfonyl)-5-((triisopropylsilyl)ethynyl)pyrido[2,3-d]pyrimidin-7(8H)-one

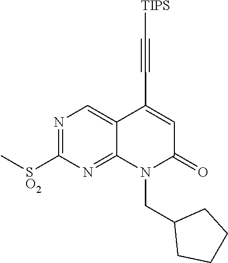

General procedure 10 was applied to 8-(cyclopentylmethyl)-2-(methylthio)-5-((triisopropylsilyl)ethynyl)pyrido[2,3-d]pyrimidin-7(8H)-one (250 mg, 0.548 mmol) with m-CPBA (284 mg, 1.65 mmol) in dichloromethane (5.5 mL) to yield the title compound (300 mg, 0.614 mmol, 100%) which was used without further purification.

(ES, m/z): [M+H]⁺=488.5.

### 8-(Cyclopentylmethyl)-2-((4-(4-methylpiperazin-1-yl)phenyl)amino)-5-((triisopropylsilyl)ethynyl)pyrido[2,3-d]pyrimidin-7(8H)-one

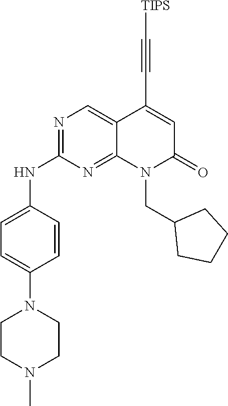

General procedure 13 was applied to 8-(cyclopentylmethyl)-2-(methylsulfonyl)-5-((triisopropylsilyl)ethynyl)pyrido[2,3-d]pyrimidin-7(8H)-one (250 mg, 0.513 mmol) with 4-(4-methylpiperazin-1-yl)aniline (108 mg, 0.564 mmol) and trifluoroacetic acid (43.0 μL, 0.564 mmol) in acetonitrile (5.0 mL). The crude material was purified by flash column chromatography eluting with methanol (0-20%) in dichloromethane to yield the title compound as an orange solid (101 mg, 0.168 mmol, 33%).

(ES, m/z): [M+H]⁺=599.7

### 8-(Cyclopentylmethyl)-5-ethynyl-2-((4-(4-methylpiperazin-1-yl)phenyl)amino)pyrido[2,3-d]pyrimidin-7(8H)-one

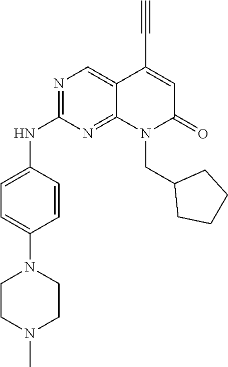 [SMILES: C#Cc1cc(=O)n(CC2CCCC2)c2nc(Nc3ccc(N4CCN(C)CC4)cc3)ncc12]

General procedure 15 was applied to 8-(cyclopentylmethyl)-2-((4-(4-methylpiperazin-1-yl)phenyl)amino)-5-((triisopropylsilyl)ethynyl)pyrido[2,3-d]pyrimidin-7(8H)-one (100 mg, 0.167 mmol) with potassium fluoride (194 mg, 3.34 mmol) in DMF (1.0 mL). The crude material was purified by flash column chromatography eluting with methanol (0-20%) in dichloromethane to yield the title compound as a red solid (62.6 mg, 0.141 mmol, 85%).

(ES, m/z): [M+H]⁺=443.5.

¹H NMR (500 MHz, CDCl₃) δ 8.76 (s, 1H), 7.50 (d, J=9.0 Hz, 2H), 7.23 (s, 1H), 6.88 (d, J=9.0 Hz, 2H), 6.56 (s, 1H), 4.26 (d, J=7.6 Hz, 2H), 3.53 (s, 1H), 3.35 (s, 4H), 2.90 (s, 4H), 2.55 (s, 3H), 2.51-2.40 (m, 1H), 1.64-1.54 (m, 5H), 1.49-1.26 (m, 3H).

### Example 68

### 8-Cyclohexyl-2-(methylthio)-5-((triisopropylsilyl)ethynyl)pyrido[2,3-d]pyrimidin-7(8H)-one

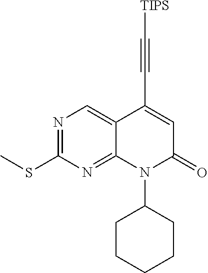

General procedure 18 was applied to 2-(methylthio)-5-((triisopropylsilyl)ethynyl)pyrido[2,3-d]pyrimidin-7(8H)-one (1.00 g, 2.68 mmol) with cyclohexanol (560 μL, 5.36 mmol), triphenylphosphine (1.05 g, 4.01 mmol) and DEAD (0.641.0 mL, 4.01 mmol) in THF (7.0 mL). The crude material was purified by flash column chromatography eluting with ethyl acetate (0-2%) in 40-60 petroleum ether to yield the title compound as a light yellow solid (428 mg, 0.940 mmol, 35%).

(ES, m/z): [M+H]⁺=456.4.

### 8-Cyclohexyl-2-(methylsulfonyl)-5-((triisopropylsilyl)ethynyl)pyrido[2,3-d]pyrimidin-7(8H)-one

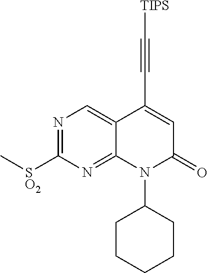

General procedure 10 was applied to 8-cyclohexyl-2-(methylthio)-5-((triisopropylsilyl)ethynyl)pyrido[2,3-d]pyrimidin-7(8H)-one (400 mg, 0.878 mmol) with m-CPBA (454 mg, 2.63 mmol) in dichloromethane (9.0 mL) to yield the title compound (420 mg, 0.862 mmol, 98%) which was carried forward without further purification.

(ES, m/z): [M+H]⁺=488.5.

### 8-Cyclohexyl-2-((4-(4-methylpiperazin-1-yl)phenyl)amino)-5-((triisopropylsilyl)ethynyl)pyrido[2,3-d]pyrimidin-7(8H)-one

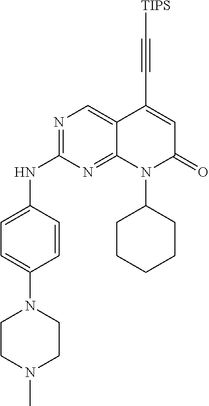

General procedure 13 was applied to 8-cyclohexyl-2-(methylsulfonyl)-5-((triisopropylsilyl)ethynyl)pyrido[2,3-d]pyrimidin-7(8H)-one (400 mg, 0.820 mmol) with 4-(4-methylpiperazin-1-yl)aniline (235 mg, 1.23 mmol) and trifluoroacetic acid (94.0 μL, 1.23 mmol) in acetonitrile (8.0 mL). The crude material was purified by flash column chromatography eluting with methanol (0-20%) in dichloromethane to yield the title compound as an orange solid (315 mg, 0.526 mmol, 64%).

(ES, m/z): [M+H]⁺=599.7.

### 8-Cyclohexyl-5-ethynyl-2-((4-(4-methylpiperazin-1-yl)phenyl)amino)pyrido[2,3-d]pyrimidin-7(8H)-one

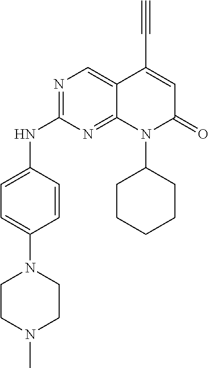 [SMILES: C#Cc1cc(=O)n(C2CCCCC2)c2nc(Nc3ccc(N4CCN(C)CC4)cc3)ncc12]

General procedure 15 was applied to 8-cyclohexyl-2-((4-(4-methylpiperazin-1-yl)phenyl)amino)-5-((triisopropylsilyl)ethynyl)pyrido[2,3-d]pyrimidin-7(8H)-one (150 mg, 0.250 mmol) with potassium fluoride (291 mg, 5.01 mmol) in DMF (1.25 mL). The crude material was purified by flash column chromatography eluting with methanol (0-20%) in dichloromethane to yield the title compound as a red solid (75.1 mg, 0.170 mmol, 68%).

(ES, m/z): [M+H]⁺=443.4.

¹H NMR (500 MHz, CDCl₃) δ 8.74 (s, 1H), 7.42 (s, 3H), 6.89 (d, J=9.0 Hz, 2H), 6.49 (s, 1H), 3.51 (s, 1H), 3.21 (t, J=5.0 Hz, 4H), 2.67 (s, 4H), 2.53 (d, J=13.3 Hz, 3H), 2.39 (s, 3H), 1.68 (d, 6H), 1.30 (t, J=13.0 Hz, 2H).

### Example 69

### 2-(Methylsulfonyl)-8-(tetrahydro-2H-pyran-4-yl)-5-((triisopropylsilyl)ethynyl)pyrido[2,3-d]pyrimidin-7(8H)-one

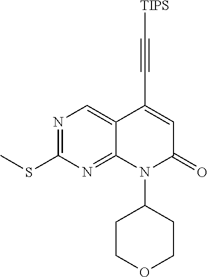

General procedure 18 was applied to 2-(methylthio)-5-((triisopropylsilyl)ethynyl)pyrido[2,3-d]pyrimidin-7(8H)-one (1.50 g, 4.01 mmol) with tetrahydro-2H-pyran-4-ol (420 μL, 4.42 mmol), triphenylphosphine (2.11 g, 8.03 mmol) and DIAD (1.58 mL, 8.03 mmol) in THF (8.0 mL). The crude material was purified by flash column chromatography eluting with ethyl acetate (0-30%) in 40-60 petroleum ether to yield the title compound as a pale yellow solid (447 mg, 0.977 mmol, 24%).

(ES, m/z): [M+H]⁺=458.4.

### 2-(Methylsulfonyl)-8-(tetrahydro-2H-pyran-4-yl)-5-((triisopropylsilyl)ethynyl)pyrido[2,3-d]pyrimidin-7(8H)-one

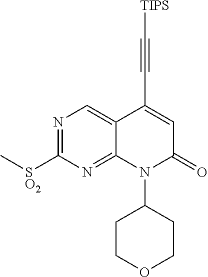

General procedure 10 was applied to 2-(methylsulfonyl)-8-(tetrahydro-2H-pyran-4-yl)-5-((triisopropylsilyl)ethynyl)pyrido[2,3-d]pyrimidin-7(8H)-one (400 mg, 0.871 mmol) with m-CPBA (452 mg, 2.62 mmol) in dichloromethane (9.0 mL) to yield the title compound (401 mg, 0.819 mmol, 94%) which was carried forward without further purification.

(ES, m/z): [M+HH]⁺=490.4.

### 2-((4-(4-Methylpiperazin-1-yl)phenyl)amino)-8-(tetrahydro-2H-pyran-4-yl)-5-((triisopropylsilyl)ethynyl)pyrido[2,3-d]pyrimidin-7(8H)-one

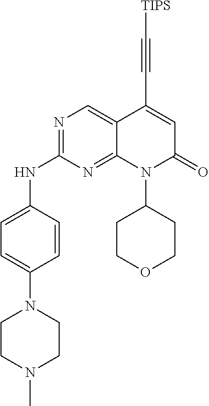

General procedure 13 was applied to 8-(2-hydroxycyclopentyl)-2-(methylsulfonyl)-5-((triisopropylsilyl)ethynyl)pyrido[2,3-d]pyrimidin-7(8H)-one (390 mg, 0.796 mmol) with 4-(4-methylpiperazin-1-yl)aniline (228 mg, 1.19 mmol) and trifluoroacetic acid (91.0 μL, 1.19 mmol) in acetonitrile (8.0 mL). The crude material was purified by flash column chromatography eluting with methanol (0-20%) in dichloromethane to yield the title compound as an orange solid (134 mg, 0.222 mmol, 28%).

(ES, m/z): [M+H]⁺=601.7

### 5-Ethynyl-2-((4-(4-methylpiperazin-1-yl)phenyl)amino)-8-(tetrahydro-2H-pyran-4-yl)pyrido[2,3-d]pyrimidin-7(8H)-one

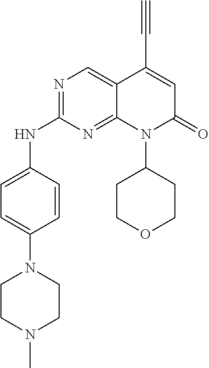 [SMILES: C#Cc1cc(=O)n(C2CCOCC2)c2nc(Nc3ccc(N4CCN(C)CC4)cc3)ncc12]

General procedure 15 was applied to 2-((4-(4-methylpiperazin-1-yl)phenyl)amino)-8-(tetrahydro-2H-pyran-4-yl)-5-((triisopropylsilyl)ethynyl)pyrido[2,3-d]pyrimidin-7(8H)-one (130 mg, 0.216 mmol) with potassium fluoride (251 mg, 4.33 mmol) in DMF (1.0 mL). The crude material was purified by flash column chromatography eluting with methanol (0-20%) in dichloromethane to yield the title compound as an red solid (68.7 mg, 0.155 mmol, 72%).

(ES, m/z): [M+H]⁺=445.4.

¹H NMR (500 MHz, CDCl₃) δ 8.86 (s, 1H), 7.56 (d, J=8.9 Hz, 2H), 7.31 (s, 1H), 7.00 (d, J=9.0 Hz, 2H), 6.60 (s, 1H), 5.69-5.49 (m, 1H), 4.14 (dd, J=11.4, 4.6 Hz, 2H), 3.62 (s, 1H), 3.55 (t, J=11.8 Hz, 2H), 3.34 (s, 4H), 3.13 (d, J=13.1 Hz, 2H), 2.80 (s, 4H), 2.52 (s, 3H), 1.57 (d, J=12.3 Hz, 2H).

### Example 70

### 2-(Methylthio)-8-((tetrahydrofuran-3-yl)methyl)-5-((triisopropylsilyl)ethynyl)pyrido[2,3-d]pyrimidin-7(8H)-one

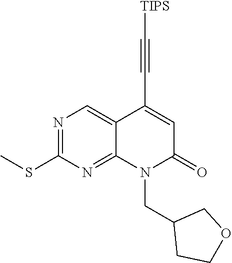

General procedure 18 was applied to 2-(methylthio)-5-((triisopropylsilyl)ethynyl)pyrido[2,3-d]pyrimidin-7(8H)-one (500 mg, 1.34 mmol) with oxolan-3-yl methanol (156 μL, 1.61 mmol), triphenylphosphine (527 mg, 2.01 mmol) and DIAD (0.400 mL, 2.01 mmol) in dichloromethane (14 mL). The crude material was purified by flash column chromatography eluting with ethyl acetate (0-30%) in 40-60 petroleum ether to yield the title compound as a colourless oil (410 mg, 0.896 mmol, 67%).

(ES, m/z): [M+H]⁺=458.4.

### 2-(Methylsulfonyl)-8-((tetrahydrofuran-3-yl)methyl)-5-((triisopropylsilyl)ethynyl)pyrido[2,3-d]pyrimidin-7(8H)-one

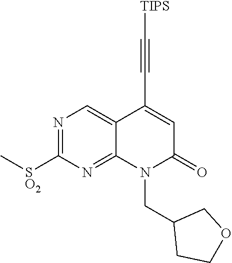

General procedure 10 was applied to 2-(methylthio)-8-((tetrahydrofuran-3-yl)methyl)-5-((triisopropylsilyl)ethynyl)pyrido[2,3-d]pyrimidin-7(8H)-one (400 mg, 0.87 mmol) with m-CPBA (452 mg, 2.62 mmol) in dichloromethane (9.0 mL) to yield the title compound (396 mg, 0.808 mmol, 92%) which was carried forward without further purification.

(ES, m/z): [M+H]⁺=490.5

### 2-((4-(4-Methylpiperazin-1-yl)phenyl)amino)-8-((tetrahydrofuran-3-yl)methyl)-5-((triisopropylsilyl)ethynyl)pyrido[2,3-d]pyrimidin-7(8H)-one

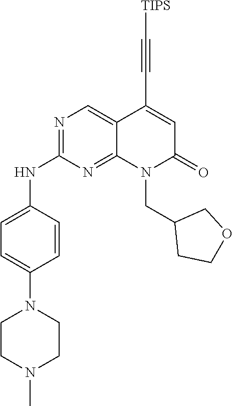

General procedure 13 was applied to 2-(methylsulfonyl)-8-((tetrahydrofuran-3-yl)methyl)-5-((triisopropylsilyl)ethynyl)pyrido[2,3-d]pyrimidin-7(8H)-one (395 mg, 0.808 mmol) with 4-(4-methylpiperazin-1-yl)aniline (232 mg, 1.21 mmol) and trifluoroacetic acid (93.0 μL, 1.21 mmol) in acetonitrile (8.0 mL). The crude material was purified by flash column chromatography eluting with methanol (0-20%) in dichloromethane to yield the title compound as an orange solid (273 mg, 56%).

(ES, m/z): [M+H]⁺=601.6.

### 5-Ethynyl-2-((4-(4-methylpiperazin-1-yl)phenyl)amino)-8-((tetrahydrofuran-3-yl)methyl)pyrido[2,3-d]pyrimidin-7(8H)-one

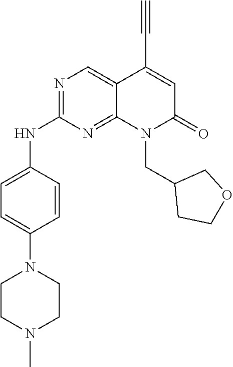 [SMILES: C#Cc1cc(=O)n(CC2CCOC2)c2nc(Nc3ccc(N4CCN(C)CC4)cc3)ncc12]

General procedure 15 was applied to 2-((4-(4-methylpiperazin-1-yl)phenyl)amino)-8-((tetrahydrofuran-3-yl)methyl)-5-((triisopropylsilyl)ethynyl)pyrido[2,3-d]pyrimidin-7(8H)-one (150 mg, 0.249 mmol) with potassium fluoride (290 mg, 4.99 mmol) in DMF (1.25 mL). The crude material was purified by flash column chromatography eluting with methanol (0-20%) in dichloromethane to yield the title compound as a red solid (99.8 mg, 0.225 mmol, 90%).

(ES, m/z): [M+H]⁺=445.4.

¹H NMR (500 MHz, CDCl₃) δ 8.87 (s, 1H), 7.54 (d, J=8.9 Hz, 2H), 7.35 (s, 1H), 6.98 (d, J=9.0 Hz, 2H), 6.65 (s, 1H), 4.47 (dd, J=12.8, 7.7 Hz, 1H), 4.36 (dd, J=12.8, 7.0 Hz, 1H), 3.95 (t, J=7.3 Hz, 1H), 3.79 (ddd, J=18.2, 8.5, 6.9 Hz, 2H), 3.71-3.66 (m, 1H), 3.65 (s, 1H), 3.40 (s, 4H), 2.91 (q, J=6.9 Hz, 4H), 2.59 (s, 3H), 1.96 (dt, J=13.5, 6.6 Hz, 1H), 1.81 (ddd, J=14.2, 12.5, 6.8 Hz, 1H).

### Example 71

### (S)-2-(Methylthio)-8-((5-oxopyrrolidin-2-yl)methyl)-5-((triisopropylsilyl)ethynyl)pyrido[2,3-d]pyrimidin-7(8H)-one

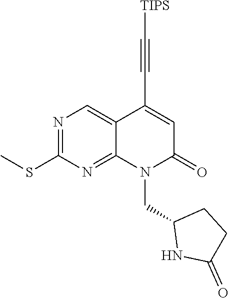

General procedure 18 was applied to 2-(methylthio)-5-((triisopropylsilyl)ethynyl)pyrido[2,3-d]pyrimidin-7(8H)-one (500 mg, 1.34 mmol) with (S)-(+)-5-(hydroxymethyl)-2-pyrrolidinone (185 mg, 1.61 mmol), triphenylphosphine (527 mg, 2.01 mmol) and DIAD (0.400 mL, 2.01 mmol) in dichloromethane (14 mL). The crude material was purified by flash column chromatography eluting with ethyl acetate (0-30%) in 40-60 petroleum ether to yield the title compound as a colourless oil (405 mg, 0.861 mmol, 64%).

(ES, m/z): [M+H]⁺=471.4.

### (S)-2-(Methylsulfonyl)-8-((5-oxopyrrolidin-2-yl)methyl)-5-((triisopropylsilyl)ethynyl)pyrido[2,3-d]pyrimidin-7(8H)-one

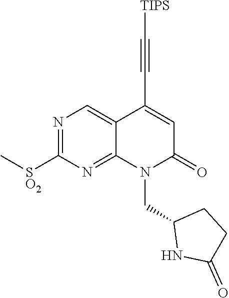

General procedure 10 was applied to (S)-2-(methylthio)-8-((5-oxopyrrolidin-2-yl)methyl)-5-((triisopropylsilyl)ethynyl)pyrido[2,3-d]pyrimidin-7(8H)-one (400 mg, 0.850 mmol) with m-CPBA (440 mg, 2.55 mmol) in dichloromethane (8.5 mL) to yield the title compound (366 mg, 0.728 mmol, 86%) which was carried forward without further purification.

(ES, m/z): [M+H]⁺=503.4.

### (S)-2-((4-(4-Methylpiperazin-1-yl)phenyl)amino)-8-((5-oxopyrrolidin-2-yl)methyl)-5-((triisopropylsilyl)ethynyl)pyrido[2,3-d]pyrimidin-7(8H)-one

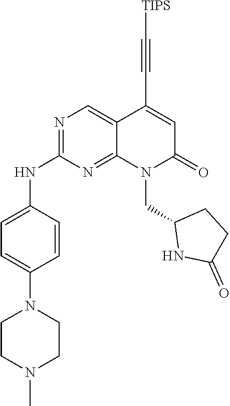

General procedure 13 was applied to (S)-2-(methylsulfonyl)-8-((5-oxopyrrolidin-2-yl)methyl)-5-((triisopropylsilyl)ethynyl)pyrido[2,3-d]pyrimidin-7(8H)-one (366 mg, 0.728 mmol) with 4-(4-methylpiperazin-1-yl)aniline (209 mg, 1.09 mmol) and trifluoroacetic acid (83.0 μL, 1.09 mmol) in acetonitrile (7.3.0 mL). The crude material was purified by flash column chromatography eluting with methanol (0-20%) in dichloromethane to yield the title compound as an orange solid (200 mg, 0.326 mmol, 45%).

(ES, m/z): [M+H]⁺=614.6.

### (S)-5-Ethynyl-2-((4-(4-methylpiperazin-1-yl)phenyl)amino)-8-((5-oxopyrrolidin-2-yl)methyl)pyrido[2,3-d]pyrimidin-7(8H)-one

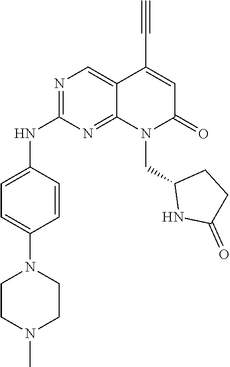 [SMILES: C#Cc1cc(=O)n(C[C@@H]2CCC(=O)N2)c2nc(Nc3ccc(N4CCN(C)CC4)cc3)ncc12]

General procedure 15 was applied to (S)-2-((4-(4-methylpiperazin-1-yl)phenyl)amino)-8-((5-oxopyrrolidin-2-yl)methyl)-5-((triisopropylsilyl)ethynyl)pyrido[2,3-d]pyrimidin-7(8H)-one (122 mg, 0.198 mmol) with potassium fluoride (231 mg, 3.97 mmol) in DMF (2.0 mL). The crude material was purified by flash column chromatography eluting with methanol (0-20%) in dichloromethane to yield the title compound as a red solid (44.5 mg, 97.4 μmol, 49%).

(ES, m/z): [M+H]⁺=458.4.

¹H NMR (500 MHz, DMSO-d₆) δ 8.75 (s, 1H), 7.74 (s, 1H), 7.55 (d, J=9.0 Hz, 2H), 6.99-6.85 (m, 2H), 6.54 (s, 1H), 5.05 (s, 1H), 4.42 (d, J=12.5 Hz, 1H), 4.21-4.01 (m, 3H), 3.09 (t, J=4.8 Hz, 4H), 2.46 (d, J=5.4 Hz, 4H), 2.23 (s, 3H), 2.16-2.01 (m, 2H), 1.88-1.66 (m, 1H).

### Example 72

### 2-(Methylthio)-8-((tetrahydrofuran-2-yl)methyl)-5-((triisopropylsilyl)ethynyl)pyrido[2,3-d]pyrimidin-7(8H)-one

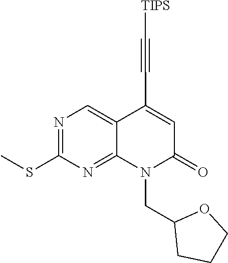

General procedure 18 was applied to 2-(methylthio)-5-((triisopropylsilyl)ethynyl)pyrido[2,3-d]pyrimidin-7(8H)-one (500 mg, 1.34 mmol) with oxolan-2-yl methanol (156 μL, 1.61 mmol), triphenylphosphine (527 mg, 2.01 mmol) and DIAD (0.400 mL, 2.01 mmol) in dichloromethane (14 mL). The crude material was purified by flash column chromatography eluting with ethyl acetate (0-30%) in 40-60 petroleum ether to yield the title compound as a colourless oil (423 mg, 0.919 mmol, 69%).

(ES, m/z): [M+H]⁺=458.4.

### 2-(Methylsulfonyl)-8-((tetrahydrofuran-2-yl)methyl)-5-((triisopropylsilyl)ethynyl)pyrido[2,3-d]pyrimidin-7(8H)-one

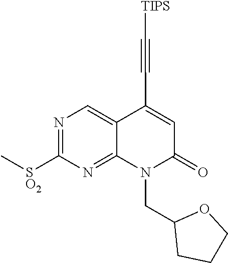

General procedure 10 was applied to 2-(methylthio)-8-((tetrahydrofuran-2-yl)methyl)-5-((triisopropylsilyl)ethynyl)pyrido[2,3-d]pyrimidin-7(8H)-one (420 mg, 0.919 mmol) with m-CPBA (476 mg, 2.78 mmol) in dichloromethane (9.0 mL) to yield the title compound (433 mg, 0.885 mmol, 96%) which was carried forward without further purification.

(ES, m/z): [M+H]⁺=490.4.

### 2-((4-(4-Methylpiperazin-1-yl)phenyl)amino)-8-((tetrahydrofuran-2-yl)methyl)-5-((triisopropylsilyl)ethynyl)pyrido[2,3-d]pyrimidin-7(8H)-one

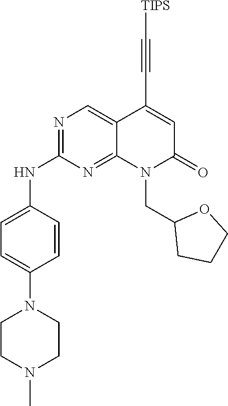

General procedure 13 was applied to 2-(methylsulfonyl)-8-((tetrahydrofuran-2-yl)methyl)-5-((triisopropylsilyl)ethynyl)pyrido[2,3-d]pyrimidin-7(8H)-one (430 mg, 0.879 mmol) with 4-(4-methylpiperazin-1-yl)aniline (252 mg, 1.32 mmol) and trifluoroacetic acid (101 μL, 1.32 mmol) in acetonitrile (9.0 mL). The crude material was purified by flash column chromatography eluting with methanol (0-20%) in dichloromethane to yield the title compound as an orange solid (310 mg, 0.516 mmol, 59%). 5-Ethynyl-2-((4-(4-methylpiperazin-1-yl)phenyl)amino)-8-((tetrahydrofuran-2-yl)methyl)pyrido[2,3-d]pyrimidin-7(8H)-one

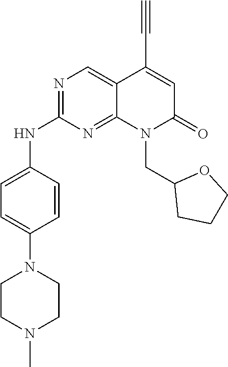 [SMILES: C#Cc1cc(=O)n(CC2CCCO2)c2nc(Nc3ccc(N4CCN(C)CC4)cc3)ncc12]

General procedure 15 was applied to 2-((4-(4-methylpiperazin-1-yl)phenyl)amino)-8-((tetrahydrofuran-2-yl)methyl)-5-((triisopropylsilyl)ethynyl)pyrido[2,3-d]pyrimidin-7(8H)-one (149 mg, 0.243 mmol) with potassium fluoride (282 mg, 4.85 mmol) in DMF (2.4 mL). The crude material was purified by flash column chromatography eluting with methanol (0-20%) in dichloromethane to yield the title compound as a red solid (62.6 mg, 0.141 mmol, 58%).

(ES, m/z): [M+H]⁺=445.4

¹H NMR (500 MHz, DMSO-d₆) δ 8.76 (s, 1H), 7.60 (d, J=8.5 Hz, 2H), 6.92 (d, J=9.1 Hz, 2H), 6.53 (s, 1H), 5.05 (s, 1H), 4.45 (dd, J=12.8, 8.1 Hz, 1H), 4.31 (s, 1H), 4.12 (dd, J=12.7, 5.1 Hz, 1H), 3.70 (s, 1H), 3.56 (d, J=7.0 Hz, 1H), 3.10 (t, J=5.0 Hz, 4H), 2.46 (t, J=5.0 Hz, 4H), 2.23 (s, 3H), 1.89 (dq, J=12.6, 6.4 Hz, 2H), 1.76 (s, 1H), 1.72-1.64 (m, 1H).

### Example 73

### 8-(2-Hydroxycyclopentyl)-2-(methylthio)-5-((triisopropylsilyl)ethynyl)pyrido[2,3-d]pyrimidin-7(8H)-one

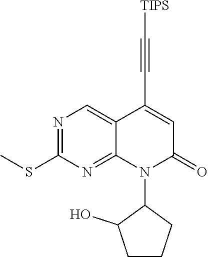

A solution of 2-(methylthio)-5-((triisopropylsilyl)ethynyl)pyrido[2,3-d]pyrimidin-7(8H)-one (620 mg, 1.66 mmol, 1.0 eq.), cyclopentene oxide (1.45 mL, 16.6 mmol, 10 eq.) and iron(III) chloride (27.0 mg, 0.166 mmol, 0.1 eq.) was stirred at 85° C. overnight. The reaction mixture was quenched with water (20 mL) and extracted with dichloromethane (3×30 mL). The combined organic layers were dried (MgSO₄) and concentrated under reduced pressure. The crude material was purified by flash column chromatography eluting with methanol (0-20%) in dichloromethane to yield the title compound as an orange solid (135 mg, 0.295 mmol, 18%).

(ES, m/z): [M+H]⁺=458.4.

### 8-(2-Hydroxycyclopentyl)-2-(methylsulfonyl)-5-((triisopropylsilyl)ethynyl)pyrido[2,3-d]pyrimidin-7(8H)-one

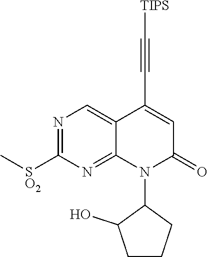

General procedure 10 was applied to 8-(2-hydroxycyclopentyl)-2-(methylthio)-5-((triisopropylsilyl)ethynyl)pyrido[2,3-d]pyrimidin-7(8H)-one (703 mg, 1.54 mmol) with mCPBA (795 mg, 4.61 mmol) in dichloromethane (15 mL) to yield the title compound (736 mg, 1.50 mmol, 98%) was carried forward without further purification.

(ES, m/z): [M+H]⁺=490.4

### 8-(2-Hydroxycyclopentyl)-2-((4-(4-methylpiperazin-1-yl)phenyl)amino)-5-((triisopropylsilyl)ethynyl)pyrido[2,3-d]pyrimidin-7(8H)-one

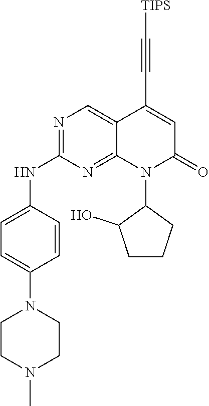

General procedure 13 was applied to 8-(2-hydroxycyclopentyl)-2-(methylsulfonyl)-5-((triisopropylsilyl)ethynyl)pyrido[2,3-d]pyrimidin-7(8H)-one (736 mg, 1.51 mmol) with 4-(4-methylpiperazin-1-yl)aniline (433 mg, 2.27 mmol) and trifluoroacetic acid (174 μL, 2.27 mmol) in acetonitrile (15 mL). The crude material was purified by flash column chromatography eluting with methanol (0-20%) in dichloromethane to yield the title compound as an orange solid (123 mg, 0.205 mmol, 14%).

(ES, m/z): [M+H]⁺=601.7.

### 5-Ethynyl-8-(2-hydroxycyclopentyl)-2-((4-(4-methylpiperazin-1-yl)phenyl)amino)pyrido[2,3-d]pyrimidin-7(8H)-one

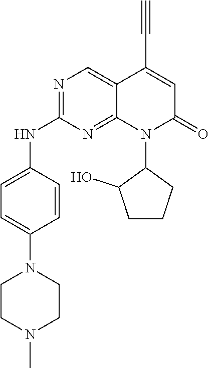 [SMILES: C#Cc1cc(=O)n(C2CCCC2O)c2nc(Nc3ccc(N4CCN(C)CC4)cc3)ncc12]

General procedure 15 was applied to 8-(2-hydroxycyclopentyl)-2-((4-(4-methylpiperazin-1-yl)phenyl)amino)-5-((triisopropylsilyl)ethynyl)pyrido[2,3-d]pyrimidin-7(8H)-one (123 mg, 0.205 mmol) with potassium fluoride (238 mg, 4.09 mmol) in DMF (2.0 mL). The crude material was purified by flash column chromatography eluting with methanol (0-20%) in dichloromethane to yield the title compound as an orange solid (20.0 mg, 22%).

(ES, m/z): [M+H]⁺=445.4.

¹H NMR (500 MHz, DMSO-d₆) δ 9.97 (s, 1H), 8.76 (s, 1H), 7.53 (d, J=8.4 Hz, 2H), 6.93 (d, J=9.1 Hz, 2H), 6.49 (s, 1H), 5.55 (s, 1H), 5.04 (s, 1H), 4.77 (s, 2H), 3.17-2.95 (m, 4H), 2.50 (d, J=2.0 Hz, 4H), 2.25 (s, 3H), 2.15-2.05 (m, 1H), 1.81 (s, 1H), 1.70 (s, 2H), 1.49 (s, 1H).

### Example 74

### Ethyl 4-(cyclopentylamino)-2-(methylsulfanyl)pyrimidine-5-carboxylate

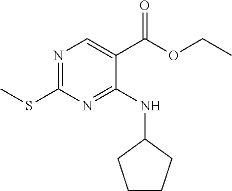 [SMILES: CCOC(=O)c1cnc(SC)nc1NC1CCCC1]

General procedure 2 was applied to ethyl 4-chloro-2-(methylsulfanyl)pyrimidine-5-carboxylate (15.0 g, 64.5 mmol), N,N-diisopropylethylamine (16.7 g, 129 mmol) and cyclopentanamine (6.59 g, 77.4 mmol) in THF (150 mL). The residue was purified by flash column chromatography, eluting with ethyl acetate (30%) in 40-60 petroleum ether to afford the title compound as an off-white solid (17.0 g, 94%).

### 4-(Cyclopentylamino)-2-(methylsulfanyl)pyrimidine-5-carboxylic acid

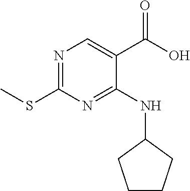 [SMILES: CSc1ncc(C(=O)O)c(NC2CCCC2)n1]

General procedure 3 was applied to ethyl 4-(cyclopentylamino)-2-(methylsulfanyl)pyrimidine-5-carboxylate (17.0 g, 60.4 mmol) with sodium hydroxide (4.83 g, 121 mmol) in water (20 mL) and THF (100 mL) to yield the title compound as a white solid (12.0 g, 78%).

### Benzotriazol-1-yl-(4-cyclopentylamino-2-methylsulfamyl-pyrimidin-5-yl)-methanone

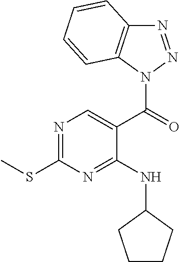 [SMILES: CSc1ncc(C(=O)n2nnc3ccccc32)c(NC2CCCC2)n1]

General procedure 4 was applied to 4-cyclopentylamino-2-methylsulfanyl-pyrimidine-5-carboxylic acid (12.0 g, 47.4 mmol), EDC (9.06 g, 47.4 mmol) and benzotriazole (5.64 g, 47.4 mmol) in dichloromethane (120 mL). The crude material was triturated with hot tert-butyl methyl ether and the mixture was left to stand overnight to yield the title compound as a white solid (13.0 g, 77%).

### Ethyl 3-(4-(cyclopentylamino)-2-(methylthio)pyrimidin-5-yl)-3-oxopropanoate

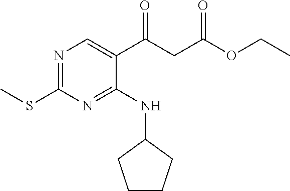 [SMILES: CCOC(=O)CC(=O)c1cnc(SC)nc1NC1CCCC1]

General procedure 5 was applied to (1H-benzo[d][1,2,3]triazol-1-yl)(4-(cyclopentylamino)-2-(methylthio)pyrimidin-5-yl)methanone (13.0 g, 36.7 mmol), ethyl acetate (8.08 g, 91.8 mmol) and 1M LiHMDS in THF (85.0 mL, 91.8 mmol) in THF (90 mL). The crude material was purified by flash column chromatography eluting with ethyl acetate (0-25%) in 40-60 petroleum ether to yield the title compound as a yellow solid (8.00 g, 67%).

### 8-Cyclopentyl-5-hydroxy-2-(methylthio)pyrido[2,3-d]pyrimidin-7(8H)-one

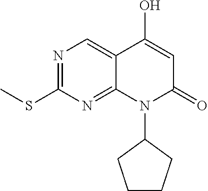 [SMILES: CSc1ncc2c(O)cc(=O)n(C3CCCC3)c2n1]

General procedure 6 was applied to ethyl 3-(4-(cyclopentylamino)-2-(methylthio)pyrimidin-5-yl)-3-oxopropanoate (4.00 g, 12.4 mmol), N,N-diisopropylethylamine (16.0 mL, 91.9 mmol) and DBU (2.00 mL, 13.4 mmol) to yield the title compound as a yellow solid (3.00 g, 88%).

### 2-(Methylthio)-7-oxo-8-phenyl-7,8-dihydropyrido[2,3-d]pyrimidin-5-yl trifluoromethanesulfonate

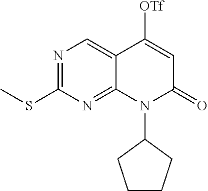 [SMILES: CSc1ncc2c(OS(=O)(=O)C(F)(F)F)cc(=O)n(C3CCCC3)c2n1]

General procedure 8 was applied to 8-cyclopentyl-5-hydroxy-2-(methylthio)pyrido[2,3-d]pyrimidin-7(8H)-one (3.00 g, 10.8 mmol), triethylamine (2.19 g, 21.7 mmol) and triflic anhydride (3.66 g, 13.0 mmol) in dichloromethane (30 mL). The crude material was purified by flash column chromatography eluting with ethyl acetate (0-50%) in 40-60 petroleum ether to yield the title compound as a light yellow solid (1.60 g, 36%).

### 2-(Methylthio)-8-phenyl-5-((triisopropylsilyl)ethynyl)pyrido[2,3-d]pyrimidin-7(8H)-one

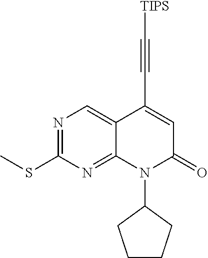

General procedure 9 was applied to 8-cyclopentyl-2-(methylthio)-7-oxo-7,8-dihydropyrido[2,3-d]pyrimidin-5-yl trifluoromethanesulfonate (1.60 g, 3.91 mmol), triisopropylsilylacetylene (1.42 g, 7.82 mmol), copper(I) iodide (74.3 mg, 0.391 mmol), bis(triphenylphosphine)palladium(II) dichloride (274 mg, 0.391 mmol) in N,N-diisopropylethylamine (5.0 mL) and DMF (10 mL). The crude material was purified by flash column chromatography eluting with ethyl acetate (0-75%) in 40-60 petroleum ether to yield the title compound as a light yellow solid (1.50 g, 87%).

### 8-Cyclopentyl-2-(methylsulfonyl)-5-((triisopropylsilyl)ethynyl)pyrido[2,3-d]pyrimidin-7(8H)-one

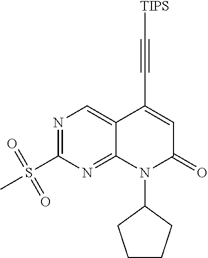

General procedure 10 was applied to 8-cyclopentyl-2-(methylthio)-5-((triisopropylsilyl)ethynyl)pyrido[2,3-d]pyrimidin-7(8H)-one (1.50 g, 3.40 mmol) and m-CPBA (1.76 g, 10.2 mmol) in dichloromethane (20 mL) to yield the title compound as a yellow solid (1.10 g, 69%) which was used in next step without further purification.

### 8-Cyclopentyl-2-((2-methoxyphenyl)amino)-5-((triisopropylsilyl)ethynyl)pyrido[2,3-d]pyrimidin-7(8H)-one

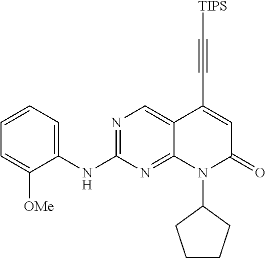

General procedure 13 was applied to 8-cyclopentyl-2-(methylsulfonyl)-5-((triisopropylsilyl)ethynyl)pyrido[2,3-d]pyrimidin-7(8H)-one (300 mg, 0.63 mmol), 2-methoxyaniline (85.8 mg, 0.698 mmol) and trifluoroacetic acid (72.3 mg, 0.63 mmol) in 2-butanol (1.0 mL). The reaction mixture was stirred at 110° C. for 24 h. The crude material was purified by reverse phase flash column chromatography eluting with acetonitrile (10-100%) in water (0.1% NH₄HCO₃) to yield the title compound as a yellow solid (140 mg, 43%).

### 8-Cyclopentyl-5-ethynyl-2-((2-methoxyphenyl)amino)pyrido[2,3-d]pyrimidin-7(8H)-one

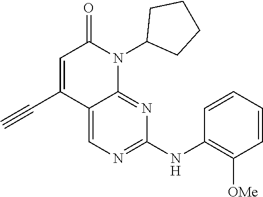 [SMILES: C#Cc1cc(=O)n(C2CCCC2)c2nc(Nc3ccccc3OC)ncc12]

General procedure 15 was applied to 2-((2-methoxyphenyl)amino)-8-phenyl-5-((triisopropylsilyl)ethynyl)pyrido [2,3-d]pyrimidin-7(8H)-one (140 mg, 0.27 mmol) and potassium fluoride (78.6 mg, 1.36 mmol) in methanol (3.0 mL) and water (1.0 mL). The reaction mixture was stirred at room temperature for 5 h and purified by reverse phase flash column chromatography eluting with acetonitrile (10-100%) in water (0.1% NH₄HCO₃). The desired fractions were collected and concentrated under reduced pressure to remove most of the acetonitrile, then lyophilized to yield the title compound as a white solid (25.0 mg, 25%).

(ES, m/z): [M+H]⁺=361

¹H NMR (300 MHz, DMSO-d₆) δ 9.04 (s, 1H), 8.75 (s, 1H), 7.65-7.63 (m, 1H), 7.27-7.17 (m, 1H), 7.11-7.08 (m, 1H), 7.0-6.93 (m, 1H), 6.49 (s, 1H), 5.70-5.61 (m, 1H), 5.01 (s, 1H), 3.79 (s, 3H), 2.27-2.15 (m, 2H), 1.72-1.60 (m, 4H), 1.45-1.21 (m, 2H).

### Example 75

### Ethyl 4-((2,4-dimethoxyphenyl)amino)-2-(methylthio)pyrimidine-5-carboxylate

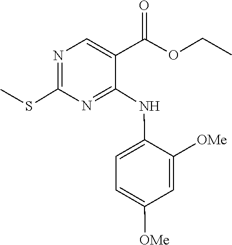 [SMILES: CCOC(=O)c1cnc(SC)nc1Nc1ccc(OC)cc1OC]

General procedure 2 was applied to ethyl 4-chloro-2-(methylthio)pyrimidine-5-carboxylate (15.0 g, 64.4 mmol), triethylamine (16.1 g, 160 mmol) and 2,4-dimethoxyaniline (11.8 g, 77.3 mmol) in THF (225 mL). The residue was slurried in 40-60 petroleum ether (100 mL), then filtered, washing with 40-60 petroleum ether and then dried under reduced pressure to yield the title compound as an off-white solid (20.0 g, 89%) which was used without further purification.

### 4-((2,4-Dimethoxyphenyl)amino)-2-(methylthio)pyrimidine-5-carboxylic acid

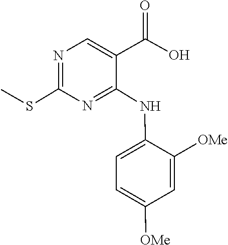 [SMILES: COc1ccc(Nc2nc(SC)ncc2C(=O)O)c(OC)c1]

General procedure 3 was applied to ethyl 4-((2,4-dimethoxyphenyl)amino)-2-(methylthio)pyrimidine-5-carboxylate (20.0 g, 57.2 mmol) in THF (600 mL) and 1M aqueous sodium hydroxide solution (600 mL) to afford the title compound as a white solid (13.0 g, 40.4 mmol, 71%).

### (1H-Benzo[d][1,2,3]triazol-1-yl)(4-((2,4-dimethoxyphenyl)amino)-2-(methylthio)pyrimidin-5-yl)methanone

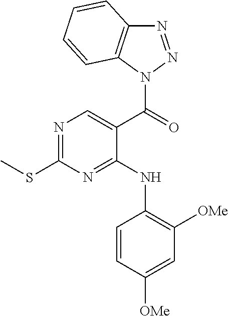 [SMILES: COc1ccc(Nc2nc(SC)ncc2C(=O)n2nnc3ccccc32)c(OC)c1]

General procedure 4 was applied to 4-((2,4-dimethoxyphenyl)amino)-2-(methylthio)pyrimidine-5-carboxylic acid (13.0 g, 40.4 mmol), EDCl (7.6.0 g, 40.4 mmol) and 1H-benzo[d][1,2,3]triazole (4.82 g, 40.4 mmol) in dichloromethane (200 mL). The residue was purified by flash column chromatography eluting with methanol (0-5%) in dichloromethane followed by reverse phase flash column chromatography eluting with acetonitrile (15-100%) in water to yield the title compound as an off-white solid (6.00 g, 14.2 mmol, 35%).

### Ethyl 3-(4-((2,4-dimethoxyphenyl)amino)-2-(methylthio)pyrimidin-5-yl)-3-oxopropanoate

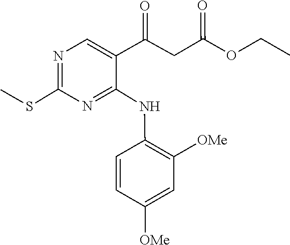 [SMILES: CCOC(=O)CC(=O)c1cnc(SC)nc1Nc1ccc(OC)cc1OC]

General procedure 5 was applied to (1H-benzo[d][1,2,3]triazol-1-yl)(4-((2,4-dimethoxyphenyl)amino)-2-(methylthio)pyrimidin-5-yl)methanone (6.00 g, 14.2 mmol), ethyl acetate (3.47 mL, 35.5 mmol) and 1M LiHMDS in THF (35.5 mL, 35.5 mmol) in THF (90 mL).

The residue was purified by flash column chromatography eluting with ethyl acetate (0-25%) in 40-60 petroleum ether to yield the title compound as a light yellow solid (1.20 g, 2.56 mmol, 18%).

### 8-(2,4-dimethoxyphenyl)-5-hydroxy-2-(methylthio)pyrido[2,3-d]pyrimidin-7(8H)-one

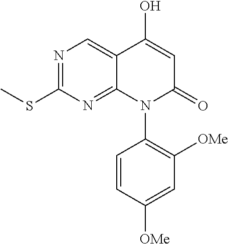 [SMILES: COc1ccc(-n2c(=O)cc(O)c3cnc(SC)nc32)c(OC)c1]

General procedure 6 was applied to 3-(4-((2,4-dimethoxyphenyl)amino)-2-(methylthio)pyrimidin-5-yl)-3-oxopropanoate (1.20 g, 2.56 mmol), N,N-diisopropylethylamine (4.0 mL) and DBU (0.50 mL) to yield the title compound as a light brown solid (800 mg, 2.32 mmol, 90%).

### 8-(2,4-dimethoxyphenyl)-2-(methylthio)-7-oxo-7,8-dihydropyrido[2,3-d]pyrimidin-5-yl trifluoromethanesulfonate

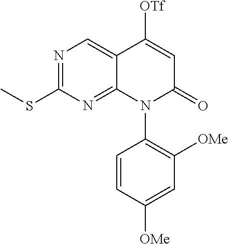 [SMILES: COc1ccc(-n2c(=O)cc(OS(=O)(=O)C(F)(F)F)c3cnc(SC)nc32)c(OC)c1]

General procedure 8 was applied to 8-(2,4-dimethoxyphenyl)-5-hydroxy-2-(methylthio)pyrido[2,3-d]pyrimidin-7(8H)-one (800 mg, 2.32 mmol), triethylamine (468 mg, 4.64 mmol) and trifluoromethanesulfonic anhydride (782 mg, 2.78 mmol) in dichloromethane (20 mL). The residue was purified by flash column chromatography eluting with ethyl acetate (0-50%) in 40-60 petroleum ether to yield the title compound as a light yellow oil (600 mg, 1.26 mmol, 54%).

### 8-(2,4-dimethoxyphenyl)-2-(methylthio)-5-((triisopropylsilyl)ethynyl)pyrido[2,3-d]pyrimidin-7(8H-one

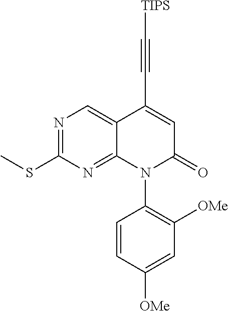

General procedure 9 was applied to 8-(2,4-dimethoxyphenyl)-2-(methylthio)-7-oxo-7,8-dihydropyrido[2,3-d]pyrimidin-5-yl trifluoromethanesulfonate (200 mg, 0.420 mmol), triisopropylsilylacetylene (155 mg, 0.840 mmol), copper(I) iodide (14.0 mg, 42.0 μmol), bis(triphenylphosphine) palladium(II) dichloride (50.0 mg, 42.0 μmol) in DMF (3.0 mL) and N,N-diisopropylethylamine (1.5 mL). The residue was purified by flash column chromatography eluting with ethyl acetate (0-75%) in 40-60 petroleum ether to yield the title compound as a light yellow solid (160 mg, 0.320 mmol, 76%).

### 8-(2,4-dimethoxyphenyl)-2-(methylsulfonyl)-5-((triisopropylsilyl)ethynyl)pyrido[2,3-d]pyrimidin-7(8H)-one

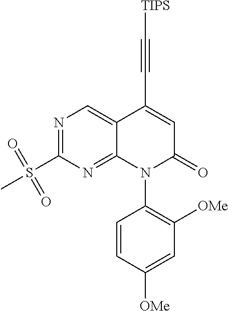

General procedure 10 was applied to 8-(2,4-dimethoxyphenyl)-2-(methylthio)-5-((triisopropylsilyl)ethynyl)pyrido[2,3-d]pyrimidin-7(8H)-one (160 mg, 0.320 mmol) and m-CPBA (236 mg, 0.960 mmol) in dichloromethane (10 mL) to yield the title compound as a yellow solid (130 mg, 0.24 mmol, 75% yield) which was used without further purification.

### 8-(2,4-Dimethoxyphenyl)-2-[(2-methoxyphenyl)amino]-5-[2-(triisopropylsilyl)ethynyl]pyrido[2,3-d]pyrimidin-7-one

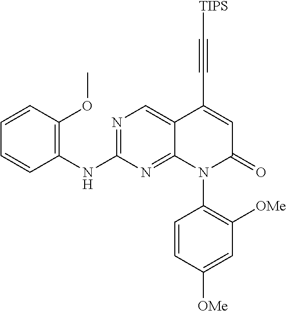

General procedure 13 was applied to 8-(2,4-dimethoxyphenyl)-2-methanesulfonyl-5-[2-(triisopropylsilyl) ethynyl]pyrido[2,3-d]pyrimidin-7-one (130 mg, 0.240 mmol), o-anisidine (29.5 mg, 0.240 mmol), trifluoroacetic acid (27.4 mg, 0.240 mmol) in 2-butanol (2.0 mL). The resulting solution was stirred for 1 h at 110° C. The crude product was purified by preparative HPLC eluting with acetonitrile (20-100%) in water (0.1% ammonia) to yield the title compound as a brown solid (120 mg, 85%)

### 8-(2,4-Dimethoxyphenyl)-5-ethynyl-2-[(2-methoxyphenyl)amino]pyrido[2,3-d]pyrimidin-7-one

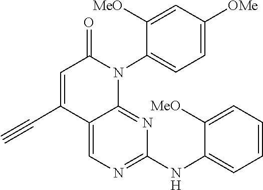 [SMILES: C#Cc1cc(=O)n(-c2ccc(OC)cc2OC)c2nc(Nc3ccccc3OC)ncc12]

H

General procedure 15 was applied to 8-(2,4-dimethoxyphenyl)-2-[(2-methoxyphenyl)amino]-5-[2-(triisopropylsilyl)ethynyl]pyrido[2,3-d]pyrimidin-7-one (120 mg, 0.205 mmol) and potassium fluoride (11.9 mg, 2.05 mmol) in THF (10 mL) and water (1.0 mL). The resulting solution was stirred overnight at room temperature. The reaction mixture was purified by reverse phase flash column chromatography eluting with acetonitrile (10-100%) in water (0.1% NH₄HCO₃) to yield the title compound as a yellow solid (16.5 mg, 19%).

(ES, m/z): [M+H]⁺=429.1.

¹H NMR (300 MHz, DMSO-d₆) δ 8.81 (s, 1H), 8.41 (s, 1H), 7.50 (d, J=8.0 Hz, 1H), 7.19 (d, J=8.6 Hz, 1H), 6.97 (q, J=4.0, 2.8 Hz, 2H), 6.81 (d, J=2.6 Hz, 1H), 6.70 (d, J=9.6 Hz, 2H), 6.52 (s, 1H), 5.13 (s, 1H), 3.89 (s, 3H), 3.82 (s, 3H), 3.64 (s, 3H).

### Example 76

### 8-Isopropyl-2-(methylthio)-5-((triisopropylsilyl)ethynyl)pyrido[2,3-d]pyrimidin-7(8H)-one

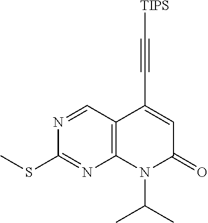

A solution 2-(methylsulfanyl)-5-[2-(triisopropylsilyl)ethynyl]-8H-pyrido[2,3-d]pyrimidin-7-one (1.20 g, 3.21 mmol), potassium carbonate (1.25 g, 9.04 mmol) and 2-iodopropane (0.630 g, 3.71 mmol) in DMF (15 mL) was stirred for 12 h at 60° C. The reaction was quenched with water (50 mL) and extracted with ethyl acetate (2×50 mL). The combined organic layers were dried (Na₂SO₄) and concentrated under reduced pressure. The crude product was purified by flash column chromatography eluting with ethyl acetate (0-75%) in 40-60 petroleum ether to yield the title compound as a light yellow solid (1.00 g, 75%).

(ES, m/z): [M+H]⁺=416.2

### 8-Isopropyl-2-methanesulfonyl-5-[2-(triisopropylsilyl)ethynyl]pyrido[2,3-d]pyrimidin-7-one

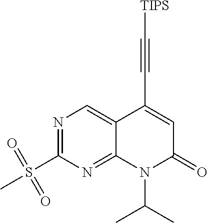

General procedure 10 was applied to 8-isopropyl-2-(methylsulfanyl)-5-[2-(triisopropylsilyl)ethynyl]pyrido[2,3-d]pyrimidin-7-one (1.00 g, 2.41 mmol) with m-CPBA (1.45 g, 8.44 mmol) in dichloromethane (20 mL) to yield the title compound as an orange oil (700 mg, 65%) which was carried forward without further purification.

(ES, m/z): [M+H]⁺=448.2

### 8-Isopropyl-2-{[4-(4-methylpiperazin-1-yl)phenyl]amino}-5-[2-(triisopropylsilyl)ethynyl]-8H-quinazolin-7-one

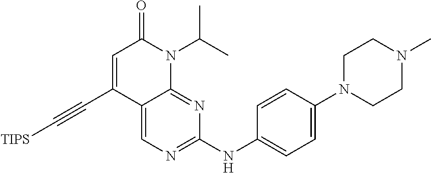

General procedure 13 was applied to 8-isopropyl-2-methanesulfonyl-5-[2-(triisopropylsilyl)ethynyl]pyrido[2,3-d]pyrimidin-7-one (700 mg, 1.56 mmol) with trifluoroacetic acid (250 mg, 2.19 mmol) and 4-(4-methylpiperazin-1-yl)aniline (350 mg, 1.83 mmol) in 2-butanol (10 mL). The resulting solution was stirred for 16 h at 100° C., diluted with methanol (15 mL) and applied onto a reverse phase column, eluting with acetonitrile (70-100%) in water (0.1% NH₄HCO₃) to yield the title compound as a dark yellow solid (420 mg, 48%).

(ES, m/z): [M+H]⁺=559.2

### 5-Ethynyl-8-isopropyl-2-{[4-(4-methylpiperazin-1-yl)phenyl]amino}pyrido[2,3-d]pyrimidin-7-one

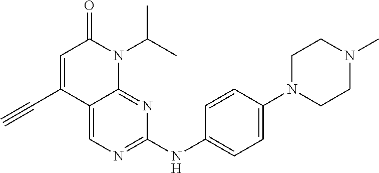 [SMILES: C#Cc1cc(=O)n(C(C)C)c2nc(Nc3ccc(N4CCN(C)CC4)cc3)ncc12]

General procedure 15 was applied to 8-isopropyl-2-{[4-(4-methylpiperazin-1-yl)phenyl]amino}-5-[2-(triisopropylsilyl)ethynyl]pyrido[2,3-d]pyrimidin-7-one (120 mg, 0.222 mmol) with potassium fluoride (150 mg, 2.58 mmol) in THF (2.0 mL), methanol (4.0 mL) and water (2.0 mL). The resulting mixture was concentrated under reduced pressure, diluted with acetonitrile and water and purified by reverse phase flash column chromatography, eluting with acetonitrile (70-100%) in water (0.1% NH₄HCO₃). The combined fractions were lyophilized to yield the title compound as an orange solid (40.0 mg, 45%).

(ES, m/z): [M+H]⁺=403.2.

¹H NMR (300 MHz, MeOD-d₄) δ 8.86 (s, 1H), 7.61 (d, J=8.8 Hz, 2H), 7.08 (d, J=8.9 Hz, 2H), 6.49 (s, 1H), 5.82 (s, 1H), 4.38 (s, 1H), 3.84 (d, J=13.1 Hz, 2H), 3.64 (d, J=12.1 Hz, 2H), 3.09 (d, J=15.2 Hz, 2H), 3.01 (s, 3H), 1.58 (d, J=6.9 Hz, 6H).

### Example 77

### Ethyl (R)-2-(methylthio)-4-((1-propionylpiperidin-3-yl)amino)pyrimidine-5-carboxylate

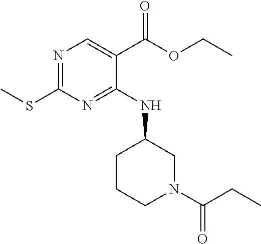 [SMILES: CCOC(=O)c1cnc(SC)nc1N[C@@H]1CCCN(C(=O)CC)C1]

General procedure 2 was applied to ethyl 4-chloro-2-(methylthio)pyrimidine-5-carboxylate (20.0 g, 85.8 mmol), triethylamine (10.8 g, 107 mmol) and (R)-1-(3-amino piperidin-1-yl)propan-1-one (6.7 g, 42.9 mmol) in THF (200 mL). The crude product was triturated with 40-60 petroleum ether (300 mL) to yield the title compound as an off-white solid (18.0 g, 59%) which was used in the next step without further purification.

(ES, m/z): [M+H]⁺=353.1.

### (R)-2-(Methylthio)-4-((1-propionylpiperidin-3-yl)amino)pyrimidine-5-carboxylic acid

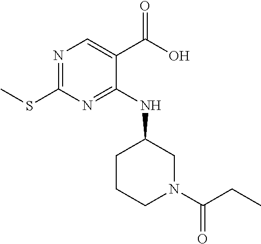 [SMILES: CCC(=O)N1CCC[C@@H](Nc2nc(SC)ncc2C(=O)O)C1]

General procedure 3 was applied to ethyl (R)-2-(methylthio)-4-((1-propionyl piperidin-3-yl)amino)pyrimidine-5-carboxylate (17.0 g, 48.2 mmol) and sodium hydroxide (9.65 g, 241 mmol) in THF (250 mL) and water (250 mL) to give the title compound as a beige solid (16.0 g, 100%).

(ES, m/z): [M+H]⁺=325.4.

### 1-[(3R)-3-{[5-(1,2,3-benzotriazole-1-carbonyl)-2-(methylsulfanyl)pyrimidin-4-yl]amino}piperidin-1-yl]propan-1-one

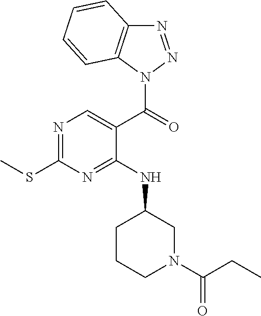 [SMILES: CCC(=O)N1CCC[C@@H](Nc2nc(SC)ncc2C(=O)n2nnc3ccccc32)C1]

General procedure 4 was applied to (R)-2-(methylthio)-4-((1-propionylpiperidin-3-yl)amino)pyrimidine-5-carboxylic acid (12.0 g, 37.0 mmol, 1.00 eq.) with 1H-benzo[d][1,2,3]triazole (8.81 g, 37.0 mmol) and EDCl (7.09 g, 37.0 mmol) in dichloromethane (120 mL). The crude product was purified by flash column chromatography eluting with ethyl acetate (5-50%) in 40-60 petroleum ether to give the title compound as a beige solid (11.0 g, 70%).

(ES, m/z): [M+H]⁺=426.0.

### Ethyl 3-[2-(methylsulfanyl)-4-{[(3R)-1-propanoylpiperidin-3-yl]amino}pyrimidin-5-yl]-3-oxopropanoate

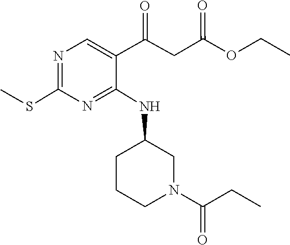 [SMILES: CCOC(=O)CC(=O)c1cnc(SC)nc1N[C@@H]1CCCN(C(=O)CC)C1]

General procedure 5 was applied to 1-[(3R)-3-{[5-(1,2,3-benzotriazole-1-carbonyl)-2-(methylsulfanyl)pyrimidin-4-yl]amino}piperidin-1-yl]propan-1-one (1.90 g, 4.46 mmol) with ethyl acetate (1.06 g, 12.1 mmol) and LiHMDS (2.02 g, 12.1 mmol) in THF (2×20 mL). The crude product was purified by flash column chromatography eluting with ethyl acetate (5-50%) in 40-60 petroleum ether to yield the title compound as a light brown solid (1.20 g, 68%).

(ES, m/z): [M+H]⁺=395.3.

### (R)-2-(Methylthio)-8-(1-propionylpiperidin-3-yl)pyrido[2,3-d]pyrimidine-5,7(6H,8H)-dione

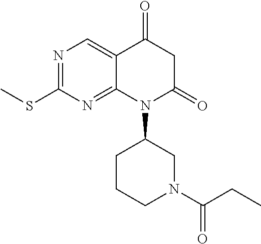 [SMILES: CCC(=O)N1CCC[C@@H](N2C(=O)CC(=O)c3cnc(SC)nc32)C1]

General procedure 6 was applied to ethyl (R)-3-(2-(methylthio)-4-((1-propionylpiperidin-3-yl)amino)pyrimidin-5-yl)-3-oxopropanoate (1.90 g, 4.82 mmol) with N,N-diisopropylethylamine (8.00 mL, 45.9 mmol) and DBU (1.00 mL, 6.69 mmol) to give the title compound as a light yellow solid (1.60 g, 95%).

(ES, m/z): [M+H]⁺=349.2.

### (R)-2-(Methylthio)-7-oxo-8-(1-propionylpiperidin-3-yl)-7,8-dihydropyrido[2,3-d]pyrimidin-5-yl trifluoromethanesulfonate

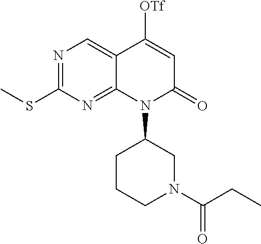 [SMILES: CCC(=O)N1CCC[C@@H](n2c(=O)cc(OS(=O)(=O)C(F)(F)F)c3cnc(SC)nc32)C1]

General procedure 8 was applied to (R)-2-(methylthio)-8-(1-propionylpiperidin-3-yl)pyrido[2,3-d]pyrimidine-5,7(6H,8H)-dione (450 mg, 1.30 mmol) with triethylamine (261 mg, 2.60 mmol) and trifluoromethanesulfonic anhydride (474 mg, 1.70 mmol) in dichloromethane (6.0 mL). The crude material was purified by reverse phase HPLC eluting with acetonitrile (5-80%) in water (0.4% formic acid) to yield the title compound as a brown solid (300 mg, 48%).

(ES, m/z): [M+H]⁺=481.1.

### (R)-2-(Methylthio)-8-(1-propionylpiperidin-3-yl)-5-((triisopropylsilyl)ethynyl)pyrido[2,3-d]pyrimidin-7(8H)-one

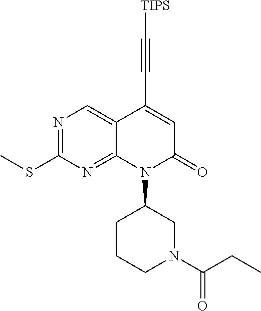

General procedure 9 was applied to (R)-2-(methylthio)-7-oxo-8-(1-propionylpiperidin-3-yl)-7,8-dihydropyrido[2,3-d]pyrimidin-5-yl trifluoromethanesulfonate (200 mg, 0.42 mmol), triisopropylsilylacetylene (152 mg, 0.840 mmol), copper(I) iodide (4.00 mg, 0.0210 mmol), bis(triphenylphosphine)palladium(II) dichloride (14.7 mg, 0.0210 mmol) and N,N-diisopropylethylamine (162 mg, 1.26 mmol) in DMF (4.5 mL). The crude product was purified by reverse phase HPLC eluting with acetonitrile (5-100%) in water (0.4% formic acid) to give the title compound as a light brown solid (150 mg, 70%).

(ES, m/z): [M+H]⁺=513.3.

### (R)-2-(Methylsulfonyl)-8-(1-propionylpiperidin-3-yl)-5-((triisopropylsilyl)ethynyl)pyrido[2,3-d]pyrimidin-7(8H)-one

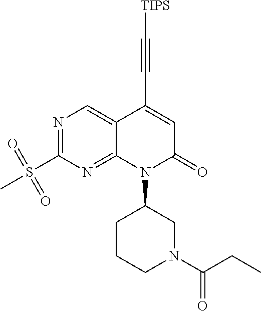

General procedure 10 was applied to (R)-2-(methylthio)-8-(1-propionylpiperidin-3-yl)-5-((triisopropylsilyl)ethynyl)pyrido[2,3-d]pyrimidin-7(8H)-one (150 mg, 0.300 mmol) with m-CPBA (216 mg, 0.888 mmol) in dichloromethane (1.0 mL). The crude material was purified by reverse phase HPLC eluting with acetonitrile (5-100%) in water (0.4% formic acid) to give the title compound as a brown solid (135 mg, 83%).

(ES, m/z): [M+H]⁺=545.3.

### (R)-2-((4-((2-(Dimethylamino)ethyl)(methyl)amino)phenyl)amino)-8-(1-propionylpiperidin-3-yl)-5-((triisopropylsilyl)ethynyl)pyrido[2,3-d]pyrimidin-7(8H)-one

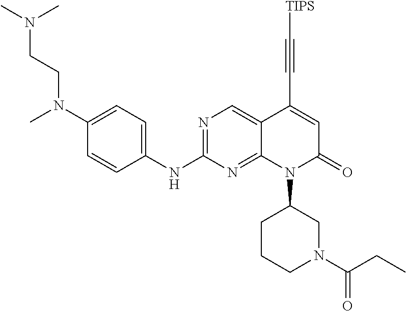

General procedure 13 was applied to (R)-2-(methylsulfonyl)-8-(1-propionylpiperidin-3-yl)-5-((triisopropylsilyl)ethynyl)pyrido[2,3-d]pyrimidin-7(8H)-one (130 mg, 0.240 mmol) with N¹-(2-(dimethylamino)ethyl)-N¹-methylbenzene-1,4-diamine (46.0 mg, 0.240 mmol) and trifluoroacetic acid (47 mg, 0.48 mmol) in 2-butanol (2.0 mL). The reaction mixture was stirred at 110° C. for 12 h. The crude product was purified by reverse phase HPLC eluting with acetonitrile (5-95%) in water (0.4% formic acid) to yield the title compound as a brown solid (105 mg, 66%).

(ES, m/z): [M+H]⁺=658.5.

### (R)-2-((4-((2-(Dimethylamino)ethyl)(methyl)amino)phenyl)amino)-5-ethynyl-8-(1-propionylpiperidin-3-yl)pyrido[2,3-d]pyrimidin-7(8H)-one trifluoroacetic acid salt

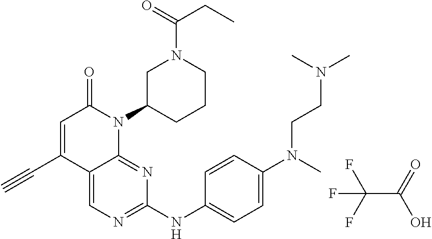

General procedure 15 was applied to (R)-2-((4-((2-(dimethylamino)ethyl)(methyl)amino)phenyl)amino)-8-(1-propionylpiperidin-3-yl)-5-((triisopropylsilyl)ethynyl)pyrido[2,3-d]pyrimidin-7(8H)-one (100 mg, 0.152 mmol) with potassium fluoride (88.3 mg, 1.52 mmol) in THF (1.5 mL) and water (100 μL). The reaction mixture was stirred at 70° C. for 5 h. The crude material was purified by reverse phase HPLC eluting with acetonitrile (5-45%) in water (0.4% trifluoroacetic acid) to afford the title compound as an orange solid.

(ES, m/z): [M+H]⁺=502.3.

¹H NMR (300 MHz, DMSO-d₆) δ 9.70 (s, 1H), 8.75 (s, 1H), 7.51-7.42 (m, 2H), 6.84-6.75 (m, 2H), 6.44 (s, 1H), 5.24 (s, 1H), 4.87 (s, 1H), 4.39 (s, 1H), 4.15-3.65 (m, 2H), 3.63-3.61 (m, 2H), 3.34-3.23 (m, 3H), 3.01-2.82 (m, 9H), 2.69 (dd, J=12.5, 3.9 Hz, 1H), 2.32 (s, 1H), 2.31-2.02 (m, 1H), 1.92-1.82 (m, 2H), 1.62-1.32 (m, 1H), 0.95 (s, 3H).

### Example 78

### Ethyl 4-(cyclopentylamino)-2-(methylsulfanyl)pyrimidine-5-carboxylate

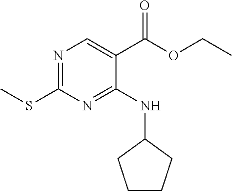 [SMILES: CCOC(=O)c1cnc(SC)nc1NC1CCCC1]

General procedure 2 was applied to ethyl 4-chloro-2-(methylsulfanyl)pyrimidine-5-carboxylate (25.0 g, 107 mmol) with triethylamine (15.0 g, 148 mmol) and cyclopentylamine (9.00 g, 106 mmol) in THF (200 mL). The resulting reaction mixture was washed with 40-60 petroleum ether (20 mL), the solids formed were collected by filtration and dried under vacuum to give the title compound as a white solid (26.6 g, 88%).

(ES, m/z): [M+H]⁺=282.3.

### 4-(Cyclopentylamino)-2-(methylsulfanyl)pyrimidine-5-carboxylic acid

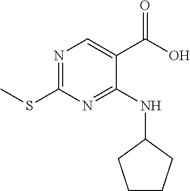 [SMILES: CSc1ncc(C(=O)O)c(NC2CCCC2)n1]

General procedure 3 was applied to ethyl 4-(cyclopentylamino)-2-(methylsulfanyl)pyrimidine-5-carboxylate (26.6 g, 94.5 mmol) with sodium hydroxide (12.0 g, 300 mmol) in THF (300 mL) and water (300 mL) to yield the title compound as a white solid (21.6 g, 90%).

(ES, m/z): [M+H]⁺=254.3.

### (1H-Benzo[d][1,2,3]triazol-1-yl)(4-(cyclopentylamino)-2-(methylthio)pyrimidin-5-yl)methanone

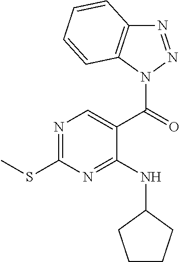 [SMILES: CSc1ncc(C(=O)n2nnc3ccccc32)c(NC2CCCC2)n1]

General procedure 4 was applied to 4-(cyclopentylamino)-2-(methylsulfanyl)pyrimidine-5-carboxylic acid (21.2 g, 83.7 mmol) with 1H-benzo[d][1,2,3]triazole (9.97 g, 83.7 mmol) and EDCl (9.97 g, 83.7 mmol) in dichloromethane (160 mL). The crude material was purified by flash column chromatography eluting with ethyl acetate (0-25%) in 40-60 petroleum ether to yield the title compound as a white solid (14.1 g, 48%).

(ES, m/z): [M+H]⁺=355.1.

### Ethyl 3-[4-(cyclopentylamino)-2-(methylsulfanyl)pyrimidin-5-yl]-3-oxopropanoate

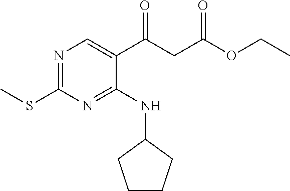 [SMILES: CCOC(=O)CC(=O)c1cnc(SC)nc1NC1CCCC1]

General procedure 5 was applied to (1H-benzo[d][1,2,3]triazol-1-yl)(4-(cyclopentylamino)-2-(methylthio)pyrimidin-5-yl)methanone (14.1 g, 39.8 mmol) with ethyl acetate (8.76 g, 99.5 mmol) and 1M LiHMDS in THF (99.0 mL, 99.0 mmol) in THF (100 & 50 mL). The crude material was purified by flash column chromatography eluting with ethyl acetate (0-25%) in 40-60 petroleum ether to yield the title compound as an orange oil (6.90 g, 54%).

(ES, m/z): [M+H]⁺=314.2.

### 8-Cyclopentyl-5-hydroxy-2-(methylthio)pyrido[2,3-d]pyrimidin-7(8H)-one

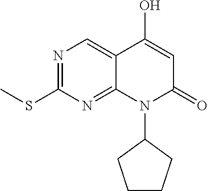 [SMILES: CSc1ncc2c(O)cc(=O)n(C3CCCC3)c2n1]

General procedure 6 was applied to ethyl 3-[4-(cyclopentylamino)-2-(methylsulfanyl)pyrimidin-5-yl]-3-oxopropanoate (6.90 g, 21.3 mmol) with N,N-diisopropylethylamine (30.0 mL, 172 mmol) and DBU (4.00 mL, 26.7 mmol) to yield the title compound as an off-white solid (5.10 g, 86%).

(ES, m/z): [M+H]⁺=278.

### 8-Cyclopentyl-2-(methylsulfanyl)-7-oxopyrido[2,3-d]pyrimidin-5-yl trifluoromethanesulfonate

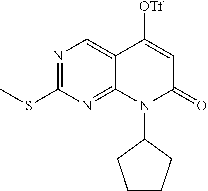 [SMILES: CSc1ncc2c(OS(=O)(=O)C(F)(F)F)cc(=O)n(C3CCCC3)c2n1]

General procedure 8 was applied to 8-cyclopentyl-5-hydroxy-2-(methylsulfanyl)pyrido[2,3-d]pyrimidin-7-one (2.05 g, 9.01 mmol) with triflic anhydride (3.05 g, 10.8 mmol) and triethylamine (2.28 g, 22.5 mmol) in dichloromethane (40 mL). The residue was purified by flash column chromatography eluting with ethyl acetate (0-20%) in 40-60 petroleum ether to yield the title compound as an orange solid (2.80 g, 22%).

(ES, m/z): [M+H]⁺=410.0

### 8-Cyclopentyl-2-(methylsulfanyl)-5-[2-(triisopropylsilyl)ethynyl]pyrido[2,3-d]pyrimidin-7-one

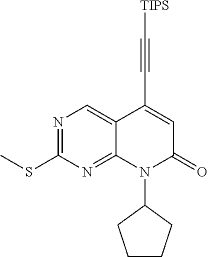

General procedure 9 was applied to 8-cyclopentyl-2-(methylsulfanyl)-7-oxopyrido[2,3-d]pyrimidin-5-yl trifluoromethanesulfonate (2.80 g, 6.84 mmol) with triisopropylsilylacetylene (2.40 g, 13.7 mmol), copper(I) iodide (250 mg, 1.37 mmol), bis(triphenylphosphine)palladium(II) dichloride (400 mg, 0.68 mmol) and N,N-diisopropylethylamine (15 mL) in DMF (30.0 mL). The crude material was purified by flash column chromatography eluting with ethyl acetate (0-75%) in 40-60 petroleum ether to yield the title compound as a light yellow solid (1.90 g, 63%).

(ES, m/z): [M+H]⁺=442.2

### 8-Cyclopentyl-2-methanesulfonyl-5-[2-(triisopropylsilyl)ethynyl]pyrido[2,3-d]pyrimidin-7-one

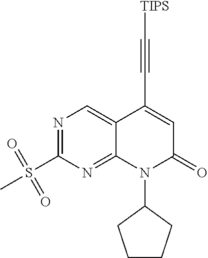

General procedure 10 was applied to 8-cyclopentyl-2-(methylsulfanyl)-5-[2-(triisopropylsilyl)ethynyl]pyrido[2,3-d]pyrimidin-7-one (1.90 g, 4.30 mmol) with m-CPBA (2.60 g, 15.1 mmol) in dichloromethane (20 mL) to yield the title compound as an orange oil (1.40 g, 69%).

(ES, m/z): [M+H]⁺=474.2.

### 8-cyclopentyl-2-((4-(4-methylpiperazin-1-yl)phenyl)amino)-5-((triisopropylsilyl)ethynyl)pyrido[2,3-d]pyrimidin-7(8H)-one

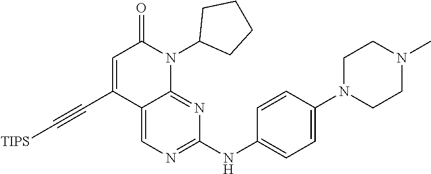

General procedure 13 was applied to 8-cyclopentyl-2-methanesulfonyl-5-[2-(triisopropylsilyl)ethynyl]pyrido[2,3-d]pyrimidin-7-one (800 mg, 1.69 mmol) with trifluoroacetic acid (192 mg, 1.69 mmol) and 4-(4-methylpiperazin-1-yl)aniline (387 mg, 2.03 mmol) in 2-butanol (10 mL). The resulting solution was stirred for 16 h at 100° C., diluted with methanol (15 mL) and applied onto a reverse phase column, eluting with acetonitrile (70-100%) in water (0.1% NH₄HCO₃) to yield the title compound as a dark yellow solid (480 mg, 49%).

(ES, m/z): [M+H]⁺=585.4.

### 8-cyclopentyl-5-ethynyl-2-((4-(4-methylpiperazin-1-yl)phenyl)amino)pyrido[2,3-d]pyrimidin-7(8H)-one

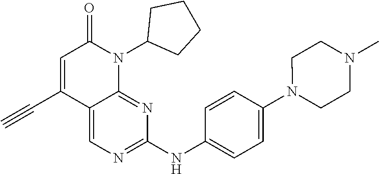 [SMILES: C#Cc1cc(=O)n(C2CCCC2)c2nc(Nc3ccc(N4CCN(C)CC4)cc3)ncc12]

General procedure 15 was applied to 8-isopropyl-2-{[4-(4-methylpiperazin-1-yl)phenyl]amino}-5-[2-(triisopropylsilyl)ethynyl]pyrido[2,3-d]pyrimidin-7-one (120 mg, 0.222 mmol) with potassium fluoride (150 mg, 2.58 mmol) in THF (2.0 mL), methanol (4.0 mL) and water (2.0 mL). The resulting mixture was stirred for 24 h at room temperature and concentrated under reduced pressure. The crude material was purified by reverse phase flash column chromatography eluting with acetonitrile (70-100%) in water (0.1% NH₄HCO₃) to yield the title compound as an orange solid (40 mg, 42%).

(ES, m/z): [M+H]⁺=429.3.

¹H NMR (300 MHz, MeOD-d₄) δ 8.85 (s, 1H), 7.52 (d, J=9.0 Hz, 2H), 7.03 (d, J=9.0 Hz, 2H), 6.49 (s, 1H), 5.94 (t, J=9.1 Hz, 1H), 4.37 (s, 1H), 3.32-3.23 (m, 5H), 2.90-2.81 (m, 5H), 2.53 (s, 3H), 2.29 (d, J=10.6 Hz, 3H), 1.94 (s, 2H), 1.85 (s, 4H), 1.63 (s, 2H), 1.32 (s, 1H).

### Example 79

### Ethyl 2-(methylsulfanyl)-4-{[(1s,4s)-4-[(tert-butoxycarbonyl)amino]cyclohexyl]amino}pyrimidine-5-carboxylate

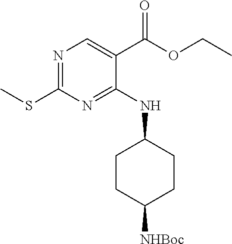 [SMILES: CCOC(=O)c1cnc(SC)nc1N[C@H]1CC[C@@H](NC(=O)OC(C)(C)C)CC1]

General procedure 2 was applied to ethyl 4-chloro-2-(methylsulfanyl)pyrimidine-5-carboxylate (30.0 g, 129 mmol) and tert-butyl N-[(1s,4s)-4-aminocyclohexyl]carbamate (27.6 g, 129 mmol) and triethylamine (13.1 g, 129 mmol) in THF (500 mL). The resulting mixture was triturated with 40-60 petroleum ether (400 mL), filtered and washed with 40-60 petroleum ether (2×200 mL). The filtrate was concentrated under reduced pressure to yield the title compound as a light brown solid (46.0 g, 87%).

(ES, m/z): [M+H]⁺=511.5.

### 2-(Methylsulfanyl)-4-{[(1s,4s)-4-[(tert-butoxycarbonyl)amino]cyclohexyl]amino}pyrimidine-5-carboxylic acid

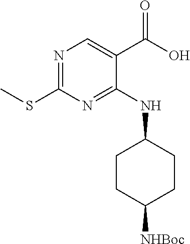 [SMILES: CSc1ncc(C(=O)O)c(N[C@H]2CC[C@@H](NC(=O)OC(C)(C)C)CC2)n1]

General procedure 3 was applied to ethyl 2-(methylsulfanyl)-4-{[(1s,4s)-4-[(tert-butoxycarbonyl)amino]cyclohexyl]amino}pyrimidine-5-carboxylate (20.0 g, 48.7 mmol) with sodium hydroxide (3.90 g, 97.4 mmol) in THF (500 mL) and water (100 mL) to yield the title compound as a white solid (18.0 g, 97%).

(ES, m/z): [M+H]⁺=383.5.

### tert-Butyl N-[(1s,4s)-4-{[5-(1,2,3-benzotriazole-1-carbonyl)-2-(methylsulfanyl) pyrimidin-4-yl]amino}cyclohexyl]carbamate

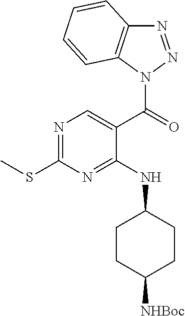 [SMILES: CSc1ncc(C(=O)n2nnc3ccccc32)c(N[C@H]2CC[C@@H](NC(=O)OC(C)(C)C)CC2)n1]

General procedure 4 was applied to 2-(methylsulfanyl)-4-{[(1s,4s)-4-[(tert-butoxycarbonyl)amino]cyclohexyl]amino}pyrimidine-5-carboxylic acid (18.0 g, 47.1 mmol) with benzotriazole (5.61 g, 47.1 mmol) and EDCl (9.02 g, 47.1 mmol) in dichloromethane (300 mL). The resulting mixture was diluted with methanol (200 mL) and filtered, washing the solids with methanol (3×30 mL). The filtrate was concentrated under reduced pressure to yield the title compound as a light brown solid (15.0 g, 66%).

(ES, m/z): [M+H]⁺=484.6.

### Ethyl 3-[2-(methylsulfanyl)-4-{[(1s,4s)-4-[(tert-butoxycarbonyl)amino]cyclohexyl]amino}pyrimidin-5-yl]-3-oxopropanoate

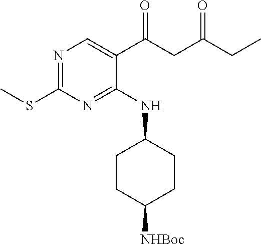 [SMILES: CCC(=O)CC(=O)c1cnc(SC)nc1N[C@H]1CC[C@@H](NC(=O)OC(C)(C)C)CC1]

General procedure 5 was applied to tert-butyl N-[(1s,4s)-4-{[5-(1,2,3-benzotriazole-1-carbonyl)-2-(methylsulfanyl)pyrimidin-4-yl]amino}cyclohexyl]carbamate (15.0 g, 31.0 mmol) with ethyl acetate (6.83 g, 77.5 mmol) and 1M LiHMDS solution in THF (77.6 mL, 77.6 mmol) in THF (200 & 50 mL). The crude product was purified by flash column chromatography eluting with ethyl acetate in 40-60 petroleum ether to yield the title compound as a light-yellow solid (10.0 g, 71%).

(ES, m/z): [M+H]⁺=437.6.

### tert-Butyl N-[(1s,4s)-4-[5-hydroxy-2-(methylsulfanyl)-7-oxopyrido[2,3-d]pyrimidin-8-yl]cyclohexyl]carbamate

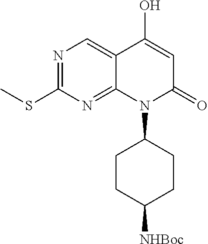 [SMILES: CSc1ncc2c(O)cc(=O)n([C@H]3CC[C@@H](NC(=O)OC(C)(C)C)CC3)c2n1]

General procedure 6 was applied to ethyl 3-[2-(methylsulfanyl)-4-{[(1s,4s)-4-[(tert-butoxycarbonyl)amino]cyclohexyl]amino}pyrimidin-5-yl]-3-oxopropanoate (10.0 g, 20.7 mmol) with N,N-diisopropylethylamine (26.7 g, 206 mmol) and DBU (6.30 g, 41.4 mmol) to yield the title compound as a yellow solid (8.00 g, 95%).

(ES, m/z): [M+H]⁺=407.5.

### tert-Butyl N-[(1s,4s)-4-[2-(methylsulfanyl)-7-oxo-5-(trifluoromethanesulfonyloxy)pyrido[2,3-d]pyrimidin-8-yl]cyclohexyl]carbamate

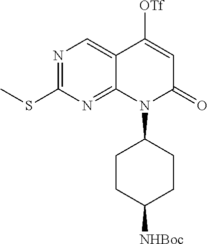 [SMILES: CSc1ncc2c(OS(=O)(=O)C(F)(F)F)cc(=O)n([C@H]3CC[C@@H](NC(=O)OC(C)(C)C)CC3)c2n1]

General procedure 8 was applied to tert-butyl N-[(1s,4s)-4-{5-hydroxy-7-oxo-2-sulfanylpyrido[2,3-d]pyrimidin-8-yl}cyclohexyl]carbamate (8.00 g, 20.4 mmol) with triflic anhydride (8.63 g, 30.6 mmol) and triethylamine (6.19 g, 61.2 mmol) in dichloromethane (50 mL). The crude material was purified by reverse phase flash column chromatography eluting with acetonitrile (20-100%) in water to yield the title compound as a brown solid (4.60 g, 42%).

(ES, m/z): [M+H]⁺=539.6.

### tert-Butyl N-[(1s,4s)-4-[2-(methylsulfanyl)-7-oxo-5-[2-(triisopropylsilyl)ethynyl]pyrido[2,3-d]pyrimidin-8-yl]cyclohexyl]carbamate

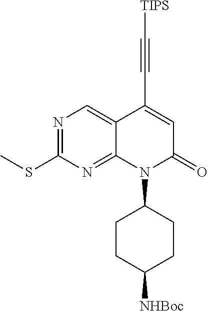

General procedure 9 was applied to tert-butyl N-[(1s,4s)-4-[7-oxo-2-sulfanyl-5-(trifluoromethanesulfonyloxy)pyrido[2,3-d]pyrimidin-8-yl]cyclohexyl]carbamate (2.00 g, 3.81 mmol) with triisopropylsilylacetylene (1.39 g, 7.63 mmol), copper(I) iodide (150 mg, 0.763 mmol), bis(triphenylphosphine)palladium(II) dichloride (270 mg, 0.381 mmol) and N,N-diisopropylethylamine (990 mg, 7.63 mmol) in DMF (10 mL). The residue was purified by reverse phase flash column chromatography eluting with acetonitrile (20-100%) in water to yield the title compound as an off-white solid (1.60 g, 73%).

(ES, m/z): [M+H]⁺=429.6.

### 2-(Methylsulfanyl)-8-[(1s,4s)-4-aminocyclohexyl]-5-[2-(triisopropylsilyl)ethynyl]pyrido[2,3-d]pyrimidin-7-one

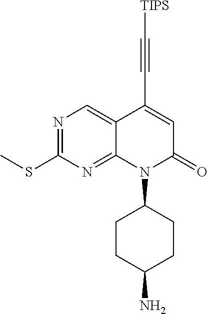

To a stirred solution of tert-butyl N-[(1s,4s)-4-[2-(methylsulfanyl)-7-oxo-5-[2-(triisopropylsilyl)ethynyl]pyrido[2,3-d]pyrimidin-8-yl]cyclohexyl]carbamate (900 mg, 1.58 mmol, 1.0 eq.) in dichloromethane (5.0 mL) was added trifluoroacetic acid (1.17 mL, 15.8 mmol, 10 eq.) at room temperature. The resulting mixture was stirred for 1 h at room temperature, concentrated under recued pressure to yield the title compound as a yellow-brown oil (800 mg, 108%).

(ES, m/z): [M+H]⁺=439.4.

### N-[(1s,4s)-4-[2-(Methylsulfanyl)-7-oxo-5-[2-(triisopropylsilyl)ethynyl]pyrido[2,3-d]pyrimidin-8-yl]cyclohexyl]acetamide

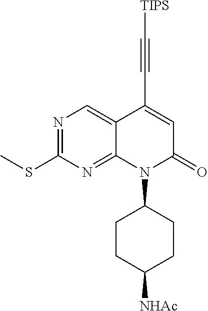

To a stirred mixture of 2-(methylsulfanyl)-8-[(1s,4s)-4-aminocyclohexyl]-5-[2-(triisopropylsilyl)ethynyl]pyrido[2,3-d]pyrimidin-7-one (800 mg, 1.70 mmol, 1.0 eq.) and triethylamine (0.47 mL, 3.40 mmol, 2.0 eq.) in dichloromethane (10 mL) was added acetic anhydride (0.18 mL, 1.87 mmol, 1.1 eq.) dropwise at 0° C. The resulting mixture was stirred for 10 min at 0° C. then warmed to room temperature. The reaction mixture was washed with water (2×5 mL) then concentrated under reduced pressure. The residue was purified by reverse phase flash column chromatography eluting with acetonitrile (20-100%) in water to yield the title compound as a yellow solid (700 mg, 80%).

(ES, m/z): [M+H]⁺=481.5.

### N-[(1s,4s)-4-{2-Methanesulfonyl-7-oxo-5-[2-(triisopropylsilyl)ethynyl]pyrido[2,3-d]pyrimidin-8-yl}cyclohexyl]acetamide

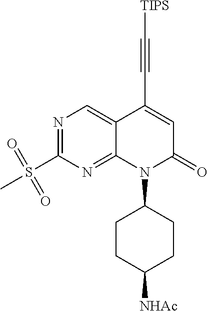

General procedure 10 was applied to N-[(1s,4s)-4-[2-(methylsulfanyl)-7-oxo-5-[2-(triisopropylsilyl)ethynyl]pyrido[2,3-d]pyrimidin-8-yl]cyclohexyl]acetamide (700 mg, 1.36 mmol) with m-CPBA (235 mg, 1.36 mmol) in dichloromethane (15 mL). The residue was purified by reverse phase flash column chromatography eluting with acetonitrile (20-100%) in water to yield the title compound as a light yellow solid (600 mg, 81%).

(ES, m/z): [M+H]⁺=545.8.

### N-[(1s,4s)-4-{2-[(4-{[2-(Dimethylamino)ethyl](methyl)amino}phenyl)amino]-7-oxo-5-[2-(triisopropylsilyl)ethynyl]pyrido[2,3-d]pyrimidin-8-yl}cyclohexyl]acetamide

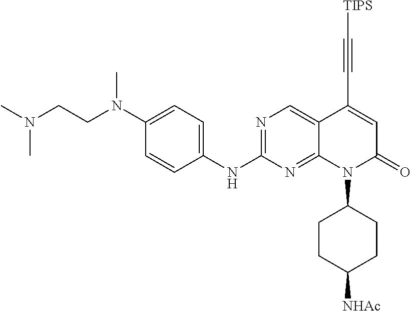

General procedure 13 was applied to N-[(1s,4s)-4-{7-oxo-2-sulfonyl-5-[2-(triisopropylsilyl)ethynyl]pyrido[2,3-d]pyrimidin-8-yl}cyclohexyl]acetamide (150 mg, 0.283 mmol) and N¹-[2-(dimethylamino)ethyl]-N¹-methylbenzene-1,4-diamine (54.6 mg, 0.283 mmol) and trifluoroacetic acid (32.2 mg, 0.283 mmol) in 2-butanol (2.0 mL). The resulting mixture was stirred for 5 h at 100° C. The crude material was purified by reverse phase flash column chromatography eluting with acetonitrile (30-100%) in water to yield the title compound as a reddish solid (120 mg, 64%).

(ES, m/z): [M+H]⁺=658.9.

### N-[(1s,4s)-4-{2-[(4-{[2-(Dimethylamino)ethyl](methyl)amino}phenyl)amino]-5-ethynyl-7-oxopyrido[2,3-d]pyrimidin-8-yl}cyclohexyl]acetamide

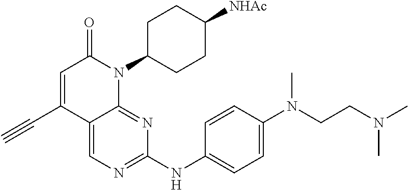 [SMILES: C#Cc1cc(=O)n([C@H]2CC[C@@H](NC(C)=O)CC2)c2nc(Nc3ccc(N(C)CCN(C)C)cc3)ncc12]

General procedure 15 was applied to N-[(1s,4s)-4-{2-[(4-{[2-(dimethylamino)ethyl](methyl)amino}phenyl)amino]-7-oxo-5-[2-(triisopropylsilyl)ethynyl]pyrido[2,3-d]pyrimidin-8-yl}cyclohexyl]acetamide (150 mg, 0.228 mmol) with potassium fluoride (132 mg, 2.28 mmol) in THF (2.0 mL). The reaction mixture was stirred for 1 h at 70° C. The residue was purified by reverse phase flash column chromatography eluting with acetonitrile (10-70%) in water to yield the title compound as a reddish solid (51.5 mg, 45%).

(ES, m/z): [M+H]⁺=502.3.

¹H NMR (300 MHz, DMSO-d₆) δ 9.96 (s, 1H), 8.72 (s, 1H), 7.76 (s, 1H), 7.50 (d, J=8.6 Hz, 2H), 6.74-6.64 (m, 2H), 6.42 (s, 1H), 5.32 (s, 1H), 5.01 (s, 1H), 3.86 (s, 1H), 3.42 (d, J=7.1 Hz, 2H), 2.90 (s, 3H), 2.42-2.34 (m, 2H), 2.18 (s, 6H), 1.93 (d, J=15.0 Hz, 5H), 1.61-1.35 (m, 4H).

### Example 80

### Ethyl 2-(methylsulfanyl)-4-{[(1s,4s)-4-{[(tert-butyldimethylsilyl)oxy]methyl}cyclohexyl]amino}pyrimidine-5-carboxylate

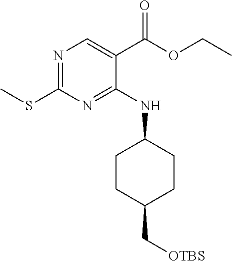 [SMILES: CCOC(=O)c1cnc(SC)nc1N[C@H]1CC[C@@H](CO[Si](C)(C)C(C)(C)C)CC1]

General procedure 2 was applied to ethyl 4-chloro-2-(methylsulfanyl)pyrimidine-5-carboxylate (20.0 g, 85.9 mmol) with (1s,4s)-4-{[(tert-butyldimethylsilyl)oxy]methyl}cyclohexan-1-amine (20.9 mL, 85.9 mmol), triethylamine (11.9 mL, 85.9 mmol) and THF (500 mL). The crude material was purified by flash column chromatography eluting with ethyl acetate (1-10%) in 40-60 petroleum ether to afford the title compound as a white oil (18.0 g, 47%).

(ES, m/z): [M+H]⁺=440.7.

### 2-(Methylsulfanyl)-4-{[(1s,4s)-4-{[(tert-butyldimethylsilyl)oxy]methyl}cyclohexyl]amino}pyrimidine-5-carboxylic acid

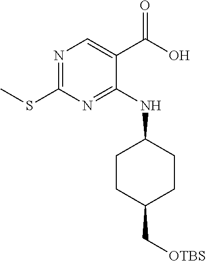 [SMILES: CSc1ncc(C(=O)O)c(N[C@H]2CC[C@@H](CO[Si](C)(C)C(C)(C)C)CC2)n1]

General procedure 3 was applied to ethyl 2-(methylsulfanyl)-4-{[(1s,4s)-4-{[(tert-butyldimethylsilyl)oxy]methyl}cyclohexyl]amino}pyrimidine-5-carboxylate (18.0 g, 40.9 mmol) with lithium hydroxide monohydrate (1.96 g, 46.7 mmol) in THF (200 mL) and water (50 mL) to yield the title compound as a white solid (10.0 g, 59%).

(ES, m/z): [M+H]⁺=412.6.

### 5-(1,2,3-Benzotriazole-1-carbonyl)-2-(methylsulfanyl)-N-[(1s,4s)-4-{[(tert-butyldimethylsilyl)oxy]methyl}cyclohexyl]pyrimidin-4-amine

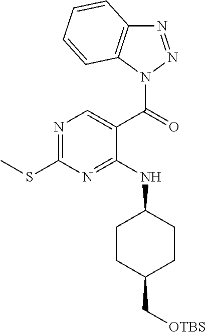 [SMILES: CSc1ncc(C(=O)n2nnc3ccccc32)c(N[C@H]2CC[C@@H](CO[Si](C)(C)C(C)(C)C)CC2)n1]

General procedure 4 was applied to 2-(methylsulfanyl)-4-{[(1s,4s)-4-{[(tert-butyldimethylsilyl)oxy]methyl}cyclohexyl]amino}pyrimidine-5-carboxylic acid (18.0 g, 43.7 mmol) with benzotriazole (5.21 g, 43.7 mmol) and EDCl (8.38 g, 43.7 mmol) in dichloromethane (200 mL). The residue was purified by flash column chromatography eluting with ethyl acetate (0-10%) in 40-60 petroleum ether to afford the title compound as an off-white oil (15.0 g, 67%).

(ES, m/z): [M+H]⁺=513.8.

### Ethyl 3-[2-(methylsulfanyl)-4-{[(1s,4s)-4-{[(tert-butyldimethylsilyl)oxy]methyl}cyclohexyl]amino}pyrimidin-5-yl]-3-oxopropanoate

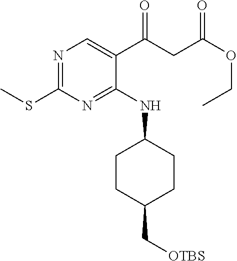 [SMILES: CCOC(=O)CC(=O)c1cnc(SC)nc1N[C@H]1CC[C@@H](CO[Si](C)(C)C(C)(C)C)CC1]

General procedure 5 was applied to 5-(1,2,3-benzotriazole-1-carbonyl)-2-(methylsulfanyl)-N-[(1s,4s)-4-{[(tert-butyldimethylsilyl)oxy]methyl}cyclohexyl]pyrimidin-4-amine (15.0 g, 29.3 mmol) with ethyl acetate (6.44 g, 73.1 mmol) and LiHMDS (12.2 g, 73.1 mmol) in THF (200 & 30 mL). The crude material was purified by flash column chromatography eluting with ethyl acetate in 40-60 petroleum ether to yield the title compound as an off-white solid (10.0 g, 71%).

(ES, m/z): [M+H]⁺=466.7.

### 5-Hydroxy-2-(methylsulfanyl)-8-[(1s,4s)-4-{[(tert-butyldimethylsilyl)oxy]methyl}cyclohexyl]pyrido[2,3-d]pyrimidin-7-one

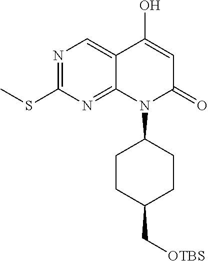 [SMILES: CSc1ncc2c(O)cc(=O)n([C@H]3CC[C@@H](CO[Si](C)(C)C(C)(C)C)CC3)c2n1]

General procedure 6 was applied to ethyl 3-[2-(methylsulfanyl)-4-{[(1s,4s)-4-{[(tert-butyldimethylsilyl)oxy]methyl}cyclohexyl]amino}pyrimidin-5-yl]-3-oxopropanoate (5.00 g, 10.4 mmol) and N,N-diisopropylethylamine (18.1.0 mL, 104 mmol), DBU (3.10 mL, 20.8 mmol) to yield the title compound as an off-white solid (4.50 g, 99%).

(ES, m/z): [M+H]⁺=436.7.

### 2-(Methylsulfanyl)-7-oxo-8-[(1s,4s)-4-{[(tert-butyldimethylsilyl)oxy]methyl}cyclohexyl]pyrido[2,3-d]pyrimidin-5-yl trifluoromethanesulfonate

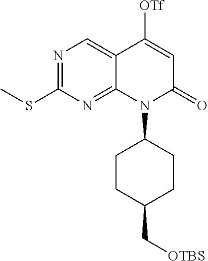 [SMILES: CSc1ncc2c(OS(=O)(=O)C(F)(F)F)cc(=O)n([C@H]3CC[C@@H](CO[Si](C)(C)C(C)(C)C)CC3)c2n1]

General procedure 8 was applied to 5-hydroxy-2-(methylsulfanyl)-8-[(1s,4s)-4-{[(tert-butyldimethylsilyl)oxy]methyl}cyclohexyl]pyrido[2,3-d]pyrimidin-7-one (1.00 g, 2.29 mmol) with triethylamine (0.32.0 mL, 2.29 mmol) and triflic anhydride (0.39 mL, 2.29 mmol) in dichloromethane (20 mL). The residue was purified by flash column chromatography eluting with ethyl acetate (1-10%) in 40-60 petroleum ether to afford the title compound as an off-white oil (800 mg, 61%).

(ES, m/z): [M+H]⁺=568.7.

### 2-(Methylsulfanyl)-8-[(1s,4s)-4-{[(tert-butyldimethylsilyl)oxy]methyl}cyclohexyl]-5-[2-(triisopropylsilyl)ethynyl]pyrido[2,3-d]pyrimidin-7-one

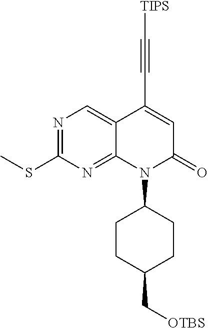

General procedure 9 was applied to 2-(methylsulfanyl)-7-oxo-8-[(1s,4s)-4-{[(tert-butyldimethylsilyl)oxy]methyl}cyclohexyl]pyrido[2,3-d]pyrimidin-5-yl trifluoromethanesulfonate (800 mg, 1.41 mmol) with triisopropylsilylacetylene (514 mg, 2.82 mmol), N,N-diisopropylethylamine (364 mg, 2.82 mmol), bis(triphenylphosphine)palladium(II) dichloride (98.9 mg, 0.141 mmol) and copper(I) iodide (26.8 mg, 0.141 mmol in DMF (5.0 mL). The residue was purified by flash column chromatography eluting with ethyl acetate (0-10%) in 40-60 petroleum ether to afford the title compound as an off-white oil (600 mg, 71%).

(ES, m/z): [M+H]⁺=601.0.

### 2-Methanesulfonyl-8-[(1s,4s)-4-{[(tert-butyldimethylsilyl)oxy]methyl}cyclohexyl]-5-[2-(triisopropyisiiyl)ethynyl]pyrido[2,3-d]pyrimidin-7-one

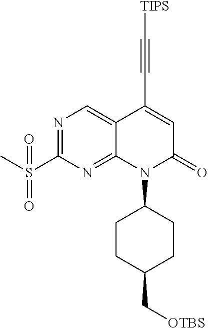

General procedure 10 was applied to 2-(methylsulfanyl)-8-[(1s,4s)-4-{[(tert-butyldimethylsilyl)oxy]methyl}cyclohexyl]-5-[2-(triisopropylsilyl)ethynyl]pyrido[2,3-d]pyrimidin-7-one (600 mg, 1.00 mmol) and m-CPBA (517 mg, 3.00 mmol) in dichloromethane (20 mL). The crude material was purified by reverse phase flash column chromatography eluting with acetonitrile (20-100%) in water to yield the title compound as a yellow-brown solid (460 mg, 73%).

(ES, m/z): [M+H]⁺=633.0.

### 2-[(4-{[2-(Dimethylamino)ethyl](methyl)amino}phenyl)amino]-8-[(1s,4s)-4-(hydroxymethyl)cyclohexyl]-5-[2-(triisopropylsilyl)ethynyl]pyrido[2,3-d]pyrimidin-7-one

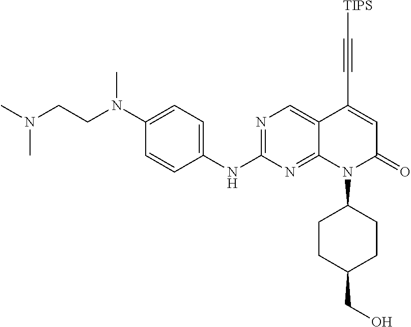

General procedure 13 was applied to 2-methanesulfonyl-8-[(1s,4s)-4-{[(tert-butyldimethylsilyl)oxy]methyl}cyclohexyl]-5-[2-(triisopropylsilyl)ethynyl]pyrido[2,3-d]pyrimidin-7-one (200 mg, 0.316 mmol) with N¹-[2-(dimethylamino)ethyl]-N¹-methylbenzene-1,4-diamine (61.2 mg, 0.316 mmol) and trifluoroacetic acid (36.1 mg, 0.316 mmol) in 2-butanol (2.0 mL). The resulting mixture was stirred for 1 h at 100° C. The crude material was purified by reverse phase flash column chromatography eluting with acetonitrile (20-100%) in water to yield the title compound as a reddish solid (120 mg, 60%).

(ES, m/z): [M+H]⁺=631.9.

### 2-[(4-{[2-(Dimethylamino)ethyl](methyl)amino}phenyl)amino]-5-ethynyl-8-[(1s,4s)-4-(hydroxymethyl)cyclohexyl]pyrido[2,3-d]pyrimidin-7-one

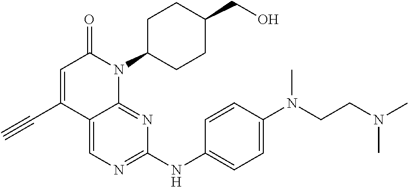 [SMILES: C#Cc1cc(=O)n([C@H]2CC[C@@H](CO)CC2)c2nc(Nc3ccc(N(C)CCN(C)C)cc3)ncc12]

General procedure 15 was applied to 2-[(4-{[2-(dimethylamino)ethyl](methyl)amino}phenyl)amino]-8-[(1s,4s)-4-(hydroxymethyl)cyclohexyl]-5-[2-(triisopropylsilyl)ethynyl]pyrido[2,3-d]pyrimidin-7-one (100 mg, 0.158 mmol) with potassium fluoride (92.1 mg, 1.58 mmol) in THF (5.0 mL). The reaction mixture was stirred for 2 h at 80° C. and purified by reverse phase flash column chromatography eluting with acetonitrile (20-80%) in water to yield the title compound as a light yellow solid (25 mg, 33%).

(ES, m/z): [M+H]⁺=475.3.

¹H NMR (300 MHz, DMSO-d₆) δ 9.93 (s, 1H), 8.71 (s, 1H), 7.49 (d, J=8.5 Hz, 2H), 6.76-6.62 (m, 2H), 6.40 (s, 1H), 5.36 (s, 1H), 5.00 (s, 1H), 4.47 (s, 1H), 3.53 (s, 2H), 3.41 (t, J=7.2 Hz, 2H), 2.90 (s, 3H), 2.59 (s, 2H), 2.42-2.32 (m, 2H), 2.18 (s, 6H), 1.87 (d, J=12.9 Hz, 2H), 1.77 (s, 1H), 1.54-1.28 (m, 4H).

### Example 81

### N-Methyl-N-[(1s,4s)-4-[2-(methylsulfanyl)-7-oxo-5-[2-(triisopropylsilyl)ethynyl]pyrido[2,3-d]pyrimidin-8-yl]cyclohexyl]propanamide

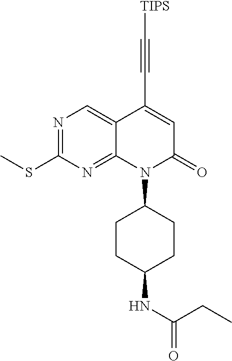

To a stirred mixture of 2-(methylsulfanyl)-8-[(1s,4s)-4-aminocyclohexyl]-5-[2-(triisopropylsilyl)ethynyl]pyrido[2,3-d]pyrimidin-7-one (800 mg, 1.70 mmol, 1.0 eq.) and triethylamine (0.59 mL, 4.25 mmol, 2.5 eq.) in dichloromethane (15 mL) was added propanoyl chloride (0.16 mL, 1.70 mmol, 1.0 eq.) dropwise at 0° C. The reaction mixture was stirred for 20 min at room temperature and quenched with water. The resulting mixture was washed with water (3×5 mL) and concentrated under reduced pressure. The crude product was purified by flash column chromatography eluting with dichloromethane (10%) in 40-60 petroleum ether to afford the title compound as an off-white solid (720 mg, 78%).

(ES, m/z): [M+H]⁺=527.8

### N-((1s,4s)-4-(2-(Methylsulfonyl)-7-oxo-5-((triisopropylsilyl)ethynyl)pyrido[2,3-d]pyrimidin-8(7H)-yl)cyclohexyl)propionamide

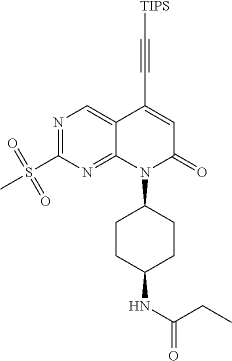

General procedure 10 was applied to N-[(1s,4s)-4-[2-(methylsulfanyl)-7-oxo-5-[2-(triisopropylsilyl)ethynyl]pyrido[2,3-d]pyrimidin-8-yl]cyclohexyl]propanamide (720 mg, 1.37 mmol) with m-CPBA (825 mg, 4.78 mmol) in dichloromethane (10 mL). The crude material was purified by reverse phase flash column chromatography eluting with acetonitrile (20-100%) in water to yield the title compound as an off-white solid (640 mg, 84%).

(ES, m/z): [M+H]⁺=559.8.

### N-((1s,4s)-4-(2-((4-(4-Methylpiperazin-1-yl)phenyl)amino)-7-oxo-5-((triisopropylsilyl)ethynyl)pyrido[2,3-d]pyrimidin-8(7H)-yl)cyclohexyl)propionamide

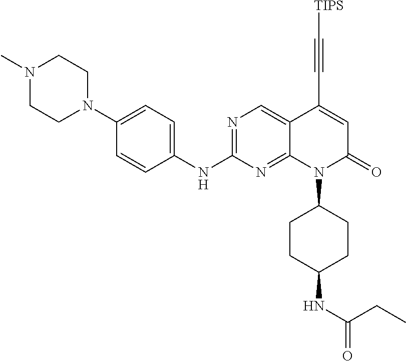

General procedure 13 was applied to N-((1s,4s)-4-(2-(methylsulfonyl)-7-oxo-5-((triisopropylsilyl)ethynyl)pyrido[2,3-d]pyrimidin-8(7H)-yl)cyclohexyl)propionamide (640 mg, 1.15 mmol) with 4-(4-methylpiperazin-1-yl)aniline (219 mg, 1.15 mmol) and trifluoroacetic acid (0.09 mL, 1.15 mmol) in 2-butanol (10 mL). The reaction mixture was stirred for 5 h at 100° C. and the crude residue was purified by reverse phase flash column chromatography eluting with acetonitrile (20-70%) in water to yield the title compound (180 mg, 0.269 mmol, 23%).

(ES, m/z): [M+H]⁺=670.9.

### N-[(1s,4s)-4-(5-Ethynyl-2-{[4-(4-methylpiperazin-1-yl)phenyl]amino}-7-oxopyrido[2,3-d]pyrimidin-8-yl)cyclohexyl]propanamide

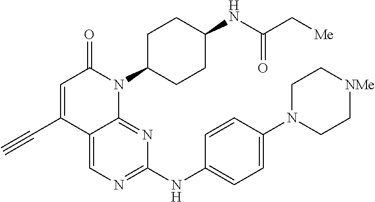 [SMILES: C#Cc1cc(=O)n([C@H]2CC[C@@H](NC(=O)CC)CC2)c2nc(Nc3ccc(N4CCN(C)CC4)cc3)ncc12]

General procedure 15 was applied to N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanamide (180 mg, 0.269 mmol) with potassium fluoride (156 mg, 2.69 mmol) in THF (4.0 mL), methanol (2.0 mL) and water (1.0 mL). The resulting mixture was stirred for 30 min at 75° C. The crude material was purified by reverse phase flash column chromatography eluting with acetonitrile (20-70%) in water to yield the title compound as a light brown solid (110 mg, 80%).

(ES, m/z): [M+H]⁺=514.4.

¹H-NMR (300 MHz, DMSO-d₆) δ 9.98 (s, 2H), 8.76 (s, 1H), 7.76-7.56 (m, 3H), 7.03 (dd, J=9.5, 2.7 Hz, 2H), 6.46 (s, 1H), 5.33 (s, 1H), 5.02 (s, 1H), 4.00-3.73 (m, 3H), 3.55 (d, J=12.0 Hz, 2H), 3.19 (s, 2H), 2.91 (d, J=17.5 Hz, 5H), 2.85-2.62 (m, 2H), 2.20 (q, J=7.6 Hz, 2H), 1.95 (d, J=13.2 Hz, 2H), 1.65-1.32 (m, 4H), 1.04 (t, J=7.6 Hz, 3H).

### Example 82

### 2-Methyl-N-[(1s,4s)-4-[2-(methylsulfanyl)-7-oxo-5-[2-(triisopropylsilyl)ethynyl]pyrido[2,3-d]pyrimidin-8-yl]cyclohexyl]propanamide

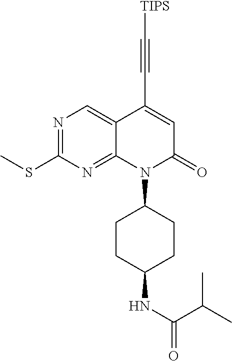

To a stirred solution of 2-(methylsulfanyl)-8-[(1s,4s)-4-aminocyclohexyl]-5-[2-(triisopropylsilyl)ethynyl]pyrido[2,3-d]pyrimidin-7-one (800 mg, 1.70 mmol, 1.0 eq.) and triethylamine (0.35 mL, 2.55 mmol, 1.5 eq.) in dichloromethane (10 mL) at 0° C. was slowly added 2-methylpropanoyl chloride (0.18 mL, 1.70 mmol, 1.0 eq.). The resulting mixture was stirred for 20 min at room temperature, washed with water (2×5 mL) and concentrated under reduced pressure. The crude residue was purified by flash column chromatography eluting with ethyl acetate (10%) in 40-60 petroleum ether to afford the title compound as an off-white solid (720 mg, 78%).

(ES, m/z): [M+H]⁺=541.8.

### 2-Methyl-N-[(1s,4s)-4-{2-methanesulfonyl-7-oxo-5-[2-(triisopropylsilyl)ethynyl]pyrido[2,3-d]pyrimidin-8-yl}cyclohexyl]propanamide

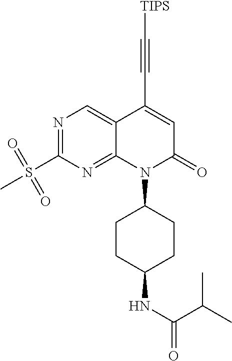

General procedure 10 was applied to 2-methyl-N-[(1s,4s)-4-[2-(methylsulfanyl)-7-oxo-5-[2-(triisopropylsilyl)ethynyl]pyrido[2,3-d]pyrimidin-8-yl]cyclohexyl]propanamide (720 mg, 1.33 mmol) and m-CPBA (804 mg, 4.66 mmol) in dichloromethane (10 mL). The crude residue was purified by reverse phase flash column chromatography eluting with acetonitrile (20-100%) in water to yield the title compound as a light yellow solid (640 mg, 84%).

(ES, m/z): [M+H]⁺=573.8.

### 2-Methyl-N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanamide

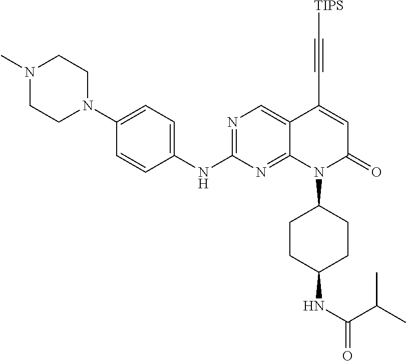

General procedure 13 was applied to 2-methanesulfonyl-8-[(1s,4s)-4-[(2-methylpropyl)amino]cyclohexyl]-5-[2-(triisopropylsilyl)ethynyl]pyrido[2,3-d]pyrimidin-7-one (640 mg, 1.15 mmol) with 4-(4-methylpiperazin-1-yl)aniline (219 mg, 1.15 mmol) and trifluoroacetic acid (0.09 mL, 1.15 mmol) in 2-butanol (10 mL). The resulting mixture was stirred for 5 h at 110° C. and purified by reverse phase flash column chromatography eluting with methanol (20-100%) in water to yield the title compound as a light brown solid (400 mg, 51%).

(ES, m/z): [M+H]⁺=685.0.

### 2-Methyl-N-[(1s,4s)-4-(5-ethynyl-2-{[4-(4-methylpiperazin-1-yl)phenyl]amino}-7-oxopyrido[2,3-d]pyrimidin-8-yl)cyclohexyl]propanamide

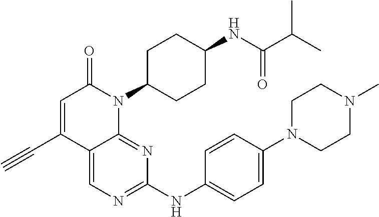 [SMILES: C#Cc1cc(=O)n([C@H]2CC[C@@H](NC(=O)C(C)C)CC2)c2nc(Nc3ccc(N4CCN(C)CC4)cc3)ncc12]

General procedure 15 was applied to 2-methyl-N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanamide (200 mg, 0.292 mmol) and potassium fluoride (170 mg, 2.92 mmol) in water (1.0 mL), methanol (2.0 mL) and THF (4.0 mL). The resulting mixture was stirred for 30 min at 75° C. and purified by reverse phase flash column chromatography eluting with methanol (20-70%) in water to yield the title compound as a light brown solid (130 mg, 84%).

(ES, m/z): [M+H]⁺=528.4

¹H-NMR (300 MHz, DMSO-d₆) δ 10.00 (d, 2H), 8.76 (s, 1H), 7.71-7.59 (m, 2H), 7.54 (s, 1H), 7.10-6.96 (m, 2H), 6.47 (s, 1H), 5.34 (s, 1H), 5.03 (s, 1H), 3.92-3.74 (m, 3H), 3.54 (d, J=12.0 Hz, 2H), 3.29-3.11 (m, 2H), 3.00-2.85 (m, 5H), 2.84-2.69 (m, 2H), 2.66-2.55 (m, 1H), 1.97 (d, J=13.7 Hz, 2H), 1.61-1.35 (m, 4H), 1.04 (d, J=6.8 Hz, 6H).

### Example 83

### Ethyl 4-(isopropylamino)-2-(methylsulfanyl)pyrimidine-5-carboxylate

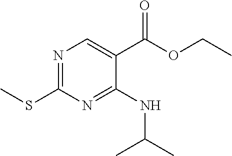 [SMILES: CCOC(=O)c1cnc(SC)nc1NC(C)C]

General procedure 2 was applied to ethyl 4-chloro-2-(methylsulfanyl)pyrimidine-5-carboxylate (15.0 g, 64.5 mmol) with triethylamine (16.3 g, 161 mmol) and isopropylamine (4.57 g, 77.4 mmol) in THF (150 mL). The crude material was triturated with 40-60 petroleum ether (20 mL) and the solids collected by filtration were dried under vacuum to yield the title compound as a white solid (15.1 g, 92%).

(ES, m/z): [M+H]⁺=256

### 4-(Isopropylamino)-2-(methylsulfanyl)pyrimidine-5-carboxylic acid

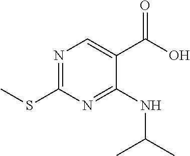

General procedure 3 was applied to ethyl 4-(isopropylamino)-2-(methylsulfanyl)pyrimidine-5-carboxylate (15.1 g, 59.1 mmol) with sodium hydroxide (12.0 g, 300 mmol) in water (300 mL) and THF (300 mL) to yield the title compound as a white solid (10.0 g, 74%).

(ES, m/z): [M+H]⁺=228

### 5-(1,2,3-Benzotriazole-1-carbonyl)-N-isopropyl-2-(methylsulfanyl)pyrimidin-4-amine

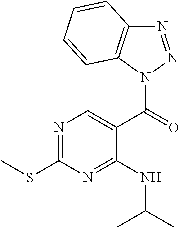 [SMILES: CSc1ncc(C(=O)n2nnc3ccccc32)c(NC(C)C)n1]

General procedure 4 was applied to 4-(isopropylamino)-2-(methylsulfanyl)pyrimidine-5-carboxylic acid (9.00 g, 39.6 mmol) with EDCl (7.59 g, 39.6 mmol) and benzotriazole (4.72 g, 39.6 mmol) in dichloromethane (100 mL). The crude material was purified by flash column chromatography, eluting with ethyl acetate (0-15%) in 40-60 petroleum ether to yield the title compound as a white solid (6.70 g, 52%).

(ES, m/z): [M+H]⁺=329

### Ethyl 3-[4-(isopropylamino)-2-(methylsulfanyl)pyrimidin-5-yl]-3-oxopropanoate

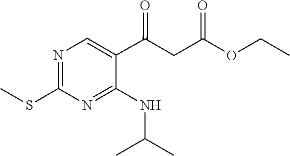 [SMILES: CCOC(=O)CC(=O)c1cnc(SC)nc1NC(C)C]

General procedure 5 was applied to 5-(1,2,3-benzotriazole-1-carbonyl)-N-isopropyl-2-(methylsulfanyl)pyrimidin-4-amine (6.03 g, 18.4 mmol) with ethyl acetate (4.30 g, 48.8 mmol) and 1M LiHMDS in THF (49.0 mL, 49.0 mmol) in THF (80 & 40 mL). The crude residue was purified by flash column chromatography, eluting with ethyl acetate (0-25%) in 40-60 petroleum ether to afford the title compound as an orange oil (4.40 g, 80%).

(ES, m/z): [M+H]⁺=298

### 5-Hydroxy-8-isopropyl-2-(methylthio)pyrido[2,3-d]pyrimidin-7(8H)-one

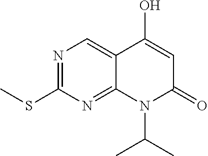 [SMILES: CSc1ncc2c(O)cc(=O)n(C(C)C)c2n1]

General procedure 6 was applied to ethyl 3-[4-(isopropylamino)-2-(methylsulfanyl)pyrimidin-5-yl]-3-oxopropanoate (2.00 g, 6.73 mmol), N,N-diisopropylethylamine (8.00 mL, 45.9 mmol) and DBU (1.00 mL, 6.69 mmol) to yield the title compound as a red brown yellow solid (1.30 g, 77%).

(ES, m/z): [M+H]⁺=250

### 8-Isopropyl-2-(methylsulfanyl)-7-oxopyrido[2,3-d]pyrimidin-5-yl trifluoromethanesulfonate

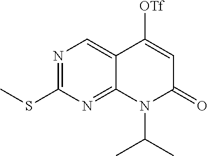 [SMILES: CSc1ncc2c(OS(=O)(=O)C(F)(F)F)cc(=O)n(C(C)C)c2n1]

General procedure 8 was applied to 5-hydroxy-8-isopropyl-2-(methylsulfanyl)pyrido[2,3-d]pyrimidin-7-one (1.20 g, 4.77 mmol) with triethylamine (1.21 g, 11.9 mmol) and trifluoromethanesulfonic anhydride (1.62 g, 5.73 mmol) in dichloromethane (50 mL). The crude material was purified by flash column chromatography eluting with ethyl acetate (0-25%) in 40-60 petroleum ether to yield the title compound as an orange solid (1.20 g, 66%).

(ES, m/z): [M+H]⁺=384

### 8-Isopropyl-2-(methylsulfanyl)-5-[2-(triisopropylsilyl)ethynyl]pyrido[2,3-d]pyrimidin-7-one

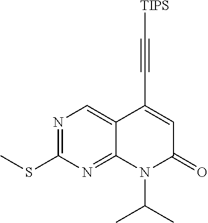

General procedure 9 was applied to 8-isopropyl-2-(methylsulfanyl)-7-oxopyrido[2,3-d]pyrimidin-5-yl trifluoromethanesulfonate (900 mg, 2.35 mmol) with triisopropylsilylacetylene (856 mg, 4.69 mmol), copper(I) iodide(I) (89.4 mg, 0.470 mmol), bis(triphenylphsophine)palladium(II) dichloride (165 mg, 0.235 mmol) in DMF (12.0 mL) and N,N-diisopropylethylamine (6.0 mL). The crude material was purified by flash column chromatography eluting with ethyl acetate (0-75%) 40-60 petroleum ether to yield the title compound as a yellow solid (900 mg, 92%).

(ES, m/z): [M+H]⁺=416

### 8-Isopropyl-2-methanesulfonyl-5-[2-(triisopropylsilyl)ethynyl]pyrido[2,3-d]pyrimidin-7-one

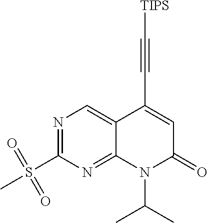

General procedure 10 was applied to 8-isopropyl-2-(methylsulfanyl)-5-[2-(triisopropylsilyl)ethynyl]pyrido[2,3-d]pyrimidin-7-one (600 mg, 1.44 mmol) with m-CPBA (872 mg, 5.05 mmol) in dichloromethane (20 mL). The crude product was purified by reverse phase flash column chromatography eluting with acetonitrile (10-100%) in water (0.1% NH₄HCO₃) to yield the title compound as an orange oil (700 mg, >100%).

(ES, m/z): [M+H]⁺=448

### 8-Isopropyl-2-[(2-methoxyphenyl)amino]-5-[2-(triisopropylsilyl)ethynyl]pyrido[2,3-d]pyrimiding-7-one

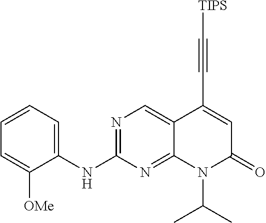

General procedure 15 was applied to 8-isopropyl-2-methanesulfonyl-5-[2-(triisopropylsilyl)ethynyl]pyrido[2,3-d]pyrimidin-7-one (250 mg, 0.558 mmol) with trifluoroacetic acid (63.7 mg, 0.558 mmol) and o-anisidine (68.8 mg, 0.558 mmol) in 2-butanol (4.0 mL). The resulting solution was stirred for 16 h at 110° C., diluted with methanol (15 mL) and applied onto a reverse phase column, eluting with acetonitrile (70-100%) in water (0.1% NH₄HCO₃) to yield the title compound as a light yellow solid (150 mg, 55%).

(ES, m/z): [M+H]⁺=491

### 5-Ethynyl-8-isopropyl-2-[(2-methoxyphenyl)amino]pyrido[2,3-d]pyrimidin-7-one

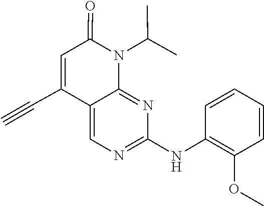 [SMILES: C#Cc1cc(=O)n(C(C)C)c2nc(Nc3ccccc3OC)ncc12]

General procedure 15 was applied to 8-isopropyl-2-[(2-methoxyphenyl)amino]-5-[2-(triisopropylsilyl)ethynyl]pyrido[2,3-d]pyrimidin-7-one (150 mg, 0.306 mmol) with potassium fluoride (1.00 g, 17.2 mmol) in THF (8.0 mL) and water (4.0 mL). The resulting solution was stirred for 2 days at 40° C. and purified by reverse phase flash column chromatography eluting with acetonitrile (20-100%) in water (0.1% NH₄HCO₃). The collected fractions were concentrated under reduced pressure to remove most of the acetonitrile and lyophilized to yield the title compound as a light yellow solid (15.0 mg, 39%).

(ES, m/z): [M+H]⁺=335

¹H NMR (300 MHz, CDCl₃) δ 8.90 (s, 1H), 8.43 (d, J=7.2 Hz, 1H), 7.94 (s, 1H), 7.13-7.02 (m, 2H), 7.02-6.92 (m, 1H), 6.60 (s, 1H), 5.87-5.76 (m, 1H), 3.97 (s, 3H), 3.62 (s, 1H), 1.65 (d, J=7.0 Hz, 6H).

### Example 84

### Ethyl 4-[(4-methoxyphenyl)amino]-2-(methylsulfanyl)pyrimidine-5-carboxylate

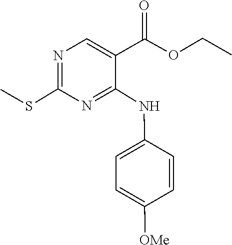 [SMILES: CCOC(=O)c1cnc(SC)nc1Nc1ccc(OC)cc1]

General procedure 2 was applied to ethyl 4-chloro-2-(methylsulfanyl)pyrimidine-5-carboxylate (35.0 g, 150 mmol) and p-anisidine (27.8 g, 226 mmol.) and N,N-diisopropylethylamine (38.9 g, 301 mmol) in THF (500 mL). The resulting mixture was diluted with 40-60 petroleum ether (1.0 μL). The precipitated solids were collected by filtration and washed with 40-60 petroleum ether (3×50 mL) to yield the title compound as a light yellow solid (36.0 g, 75%)

### 4-[(4-methoxyphenyl)amino]-2-(methylsulfanyl)pyrimidine-5-carboxylic acid

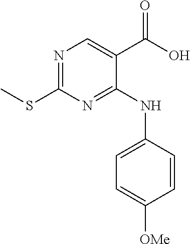 [SMILES: COc1ccc(Nc2nc(SC)ncc2C(=O)O)cc1]

General procedure 3 was applied to ethyl 4-[(4-methoxyphenyl)amino]-2-(methylsulfanyl)pyrimidine-5-carboxylate (20.0 g, 62.6 mmol) and sodium hydroxide (10.0 g, 250 mmol) in water (40 mL) and THF (400 mL) to yield the title compound as a white solid (15.1 g, 83%).

### 5-(1,2,3-benzotriazole-1-carbonyl)-N-(4-methoxyphenyl)-2-(methylsulfanyl)pyrimidin-4-amine

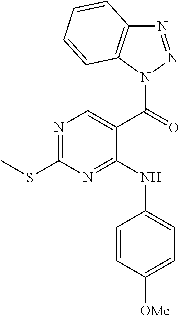 [SMILES: COc1ccc(Nc2nc(SC)ncc2C(=O)n2nnc3ccccc32)cc1]

General procedure 4 was applied to 4-[(4-methoxyphenyl)amino]-2-(methylsulfanyl)pyrimidine-5-carboxylic acid (10.0 g, 34.3 mmol), EDCl (7.90 g, 41.2 mmol) and benzotriazole (4.09 g, 34.3 mmol) in dichloromethane (300 mL). The residue was purified by flash column chromatography, eluting with ethyl acetate (9%) in 40-60 petroleum ether to afford the title compound as a light yellow solid (12.0 g, 89%).

### Ethyl 3-{4-[(4-methoxyphenyl)amino]-2-(methylsulfanyl)pyrimidin-5-yl}-3-oxopropanoate

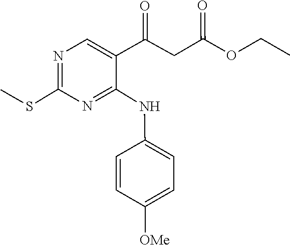 [SMILES: CCOC(=O)CC(=O)c1cnc(SC)nc1Nc1ccc(OC)cc1]

General procedure 5 was applied to 5-(1,2,3-benzotriazole-1-carbonyl)-N-(4-methoxyphenyl)-2-(methylsulfanyl)pyrimidin-4-amine (6.00 g, 15.3 mmol), ethyl acetate (3.37 g, 38.2 mmol) LiHMDS (6.40 g, 38.2 mmol) in THF (120 mL). The residue was purified by flash column chromatography, eluting with ethyl acetate (16%) in 40-60 petroleum ether to afford the title compound as a light yellow solid (3.25 g, 59%).

### 5-hydroxy-8-(4-methoxyphenyl)-2-(methylsulfanyl)pyrido[2,3-d]pyrimidin-7-one

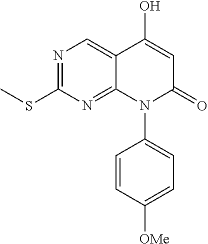 [SMILES: COc1ccc(-n2c(=O)cc(O)c3cnc(SC)nc32)cc1]

General procedure 6 was applied to ethyl 3-{4-[(4-methoxyphenyl)amino]-2-(methylsulfanyl)pyrimidin-5-yl}-3-oxopropanoate (2.96 mg, 8.19 mmol) and DBU (1.37 g, 9.01 mmol) in N,N-diisopropyl-N-ethylamine (25 mL) to yield the title compound as a brown solid (2.40 g, 93%).

### 8-(4-Methoxyphenyl)-2-(methylsulfanyl)-7-oxopyrido[2,3-d]pyrimidin-5-yl trifluoromethanesulfonate

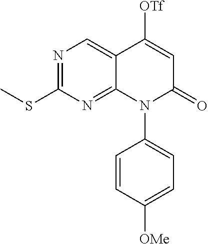 [SMILES: COc1ccc(-n2c(=O)cc(OS(=O)(=O)C(F)(F)F)c3cnc(SC)nc32)cc1]

General procedure 8 was applied to 5-hydroxy-8-(4-methoxyphenyl)-2-(methylsulfanyl)pyrido[2,3-d]pyrimidin-7-one (1.00 g, 3.17 mmol) with triflic anhydride (1.79 g, 6.34 mmol) and triethylamine (643 mg, 4.98 mmol) in dichloromethane (20 mL). The crude material was purified by flash column chromatography eluting with ethyl acetate (20%) in 40-60 petroleum ether to yield the title compound as a light yellow solid (800 mg, 56%).

(ES, m/z): [M+H]⁺=448

### 8-(4-Methoxyphenyl)-2-(methylsulfanyl)-5-[2-(triisopropylsilyl)ethynyl]pyrido[2,3-d]pyrimidin-7-one

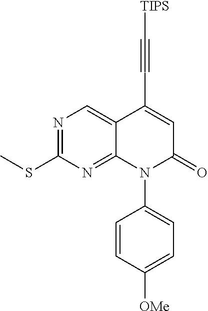

General procedure 9 was applied to 8-(4-methoxyphenyl)-2-(methylsulfanyl)-7-oxopyrido[2,3-d]pyrimidin-5-yl trifluoromethanesulfonate (800 mg, 1.79 mmol) with triisopropylsilylacetylene (653 mg, 3.58 mmol), bis(triphenylphsophine)palladium(II) dichloride (127 mg, 0.179 mmol), copper(I) iodide (35.0 mg, 0.179 mmol) in N,N-diisopropylethylamine (4.0 mL) and DMF (8.0 mL). The crude material was purified by reverse phase flash column chromatography eluting with acetonitrile (25-100%) in water (0.1% NH₄HCO₃) to yield the title compound as a yellow solid (850 mg, 99%).

(ES, m/z): [M+H]⁺=480

### 2-Methanesulfonyl-8-(4-methoxyphenyl)-5-[2-(triisopropylsilyl)ethynyl]pyrido[2,3-d]pyrimidin-7-one

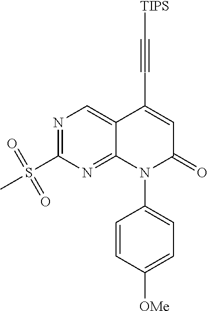

General procedure 10 was applied to 8-(4-methoxyphenyl)-2-(methylsulfanyl)-5-[2-(triisopropylsilyl)ethynyl]pyrido[2,3-d]pyrimidin-7-one (850 mg, 1.77 mmol) with m-CPBA (922 mg, 5.34 mmol) in dichloromethane (20 mL). The crude material was purified by reverse phase flash column chromatography eluting with acetonitrile (25-100%) in water (0.1% NH₄HCO₃) to yield the title compound as a light brown solid (853 mg, 94%).

(ES, m/z): [M+H]⁺=512.

### 8-(4-Methoxyphenyl)-2-[(2-methoxyphenyl)amino]-5-[2-(triisopropylsilyl)ethynyl]pyrido[2,3-d]pyrimidin-7-one

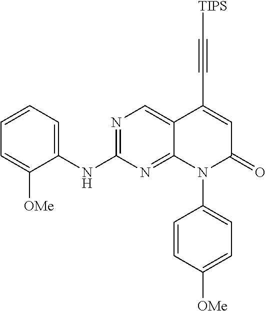

General procedure 13 was applied to 2-methanesulfonyl-8-(4-methoxyphenyl)-5-[2-(triisopropylsilyl)ethynyl]pyrido[2,3-d]pyrimidin-7-one (853 mg, 1.67 mmol) with o-anisidine (206 mg, 1.67 mmol) and trifluoroacetic acid (191 mg, 1.67 mmol) in 2-butanol (15 mL). The resulting solution was heated at 100° C. for 16 h resulting solution was diluted with dichloromethane (20 mL). The crude material was purified by reverse phase flash column chromatography eluting with acetonitrile (25-100%) in water (0.1% NH₄HCO₃) to yield the title compound as a yellow solid (525 mg, 57%).

(ES, m/z): [M+H]⁺=555

### 5-ethynyl-8-(4-methoxyphenyl)-2-[(2-methoxyphenyl)amino]pyrido[2,3-d]pyrimidin-7-one

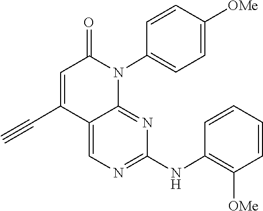 [SMILES: C#Cc1cc(=O)n(-c2ccc(OC)cc2)c2nc(Nc3ccccc3OC)ncc12]

General procedure 15 was applied to 8-(4-methoxyphenyl)-2-[(2-methoxyphenyl)amino]-5-[2-(triisopropylsilyl)ethynyl]pyrido[2,3-d]pyrimidin-7-one (525 mg, 0.950 mmol) with potassium fluoride (552 mg, 9.50 mmol) in THF/water (22.0 mL, 10:1). The resulting solution was stirred for 20 h at 40° C. The crude material was purified by reverse phase flash column chromatography eluting with acetonitrile (25-100%) in water (0.1% NH₄HCO₃) to yield the title compound as a light brown solid (350 mg 93%).

(ES, m/z): [M+H]⁺=399

¹H NMR (300 MHz, CDCl₃) δ 8.90 (s, 1H), 8.13 (s, 1H), 7.63 (s, OH), 7.26-7.21 (m, 2H), 7.18-7.12 (m, 2H), 6.94 (ddd, J=8.8, 7.4, 1.6 Hz, 1H), 6.84 (dd, J=8.2, 1.5 Hz, 1H), 6.78 (d, J=0.5 Hz, 1H), 6.55 (t, J=7.5 Hz, 1H), 3.96 (s, 3H), 3.89 (s, 3H), 3.70 (d, J=0.5 Hz, 1H).

### Example 85

### N-(3-(2-((2-Methoxyphenyl)amino)-7-oxo-5-((triisopropylsilyl)ethynyl)pyrido[2,3-d]pyrimidin-8(7H)-yl)phenyl)acrylamide

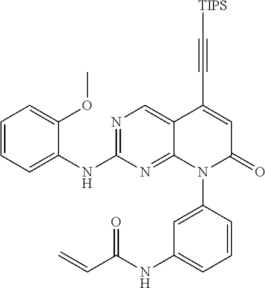

To a stirred solution of 2-((2-methoxyphenyl)amino)-8-(3-(methylamino)phenyl)-5-((triisopropylsilyl)ethynyl)pyrido[2,3-d]pyrimidin-7(8H)-one (900 mg, 0.740 mmol) in dichloromethane (14 mL) at 0° C. was added acryloyl chloride (60 μL, 0.740 mmol) dropwise. The reaction mixture was stirred at room temperature for 1 h, concentrated under reduced pressure and the crude material was purified by flash column chromatography eluting with ethyl acetate (30-70%) in 40-60 petroleum ether to yield the title compound as a yellow solid (206 mg, 47%).

(ES, m/z): [M+H]⁺=594.5.

### N-(3-(5-Ethynyl-2-((2-methoxyphenyl)amino)-7-oxopyrido[2,3-d]pyrimidin-8(7H)-yl)phenyl)acrylamide

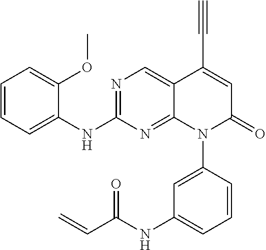 [SMILES: C#Cc1cc(=O)n(-c2cccc(NC(=O)C=C)c2)c2nc(Nc3ccccc3OC)ncc12]

General procedure 15 was applied to N-(3-(2-((2-methoxyphenyl)amino)-7-oxo-5-((triisopropylsilyl)ethynyl)pyrido[2,3-d]pyrimidin-8(7H)-yl)phenyl)acrylamide (50.0 mg, 84.0 μmol) and potassium fluoride (49.0 mg, 0.840 mmol) in DMF (1.6 mL). The reaction was stirred at room temperature for 4 h. The crude material was purified by flash column chromatography eluting with ethyl acetate (50-60%) in 40-60 petroleum ether to yield the title compound as a yellow solid (8.60 mg, 19.7 μmol, 23%).

(ES, m/z): [M+H]⁺=438.3.

¹H-NMR (500 MHz, DMSO-d₆) δ 10.38 (s, 1H), 8.83 (s, 1H), 8.45 (s, 1H), 7.85-7.68 (m, 2H), 7.59-7.42 (m, 2H), 7.10-6.97 (m, 1H), 6.72 (s, 1H), 6.44 (dd, J=16.9, 10.2 Hz, 1H), 6.25 (dd, J=17.0, 2.0 Hz, 1H), 5.83-5.64 (m, 1H), 5.15 (s, 1H), 3.81 (s, 3H).

### Example 86

### 8-(2,4-Dimethoxyphenyl)-2-[(2-methoxyphenyl)amino]-5-[2-(triisopropylsilyl)ethynyl]pyrido[2,3-d]pyrimidin-7-one

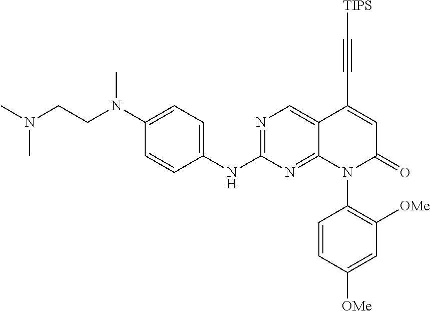

General procedure 13 was applied to 8-(2,4-dimethoxyphenyl)-2-methanesulfonyl-5-[2-(triisopropylsilyl)ethynyl]pyrido[2,3-d]pyrimidin-7-one (600 mg, 1.11 mmol) with N¹-[2-(dimethylamino)ethyl]-N¹-methylbenzene-1,4-diamine (214 mg, 1.11 mmol) and trifluoroacetic acid (126 mg, 1.11 mmol) in 2-butanol (5.0 mL). The resulting mixture was stirred for 1 h at 110° C. The residue was purified by reverse phase flash column chromatography eluting with acetonitrile (20-80%) in water to yield the title compound as a light brown solid (450 mg, 62%).

### 8-(2,4-Dimethoxyphenyl)-2-[(4-{[2-(dimethylamino)ethyl](methyl)amino}phenyl)amino]-5-ethynylpyrido[2,3-d]pyrimidin-7-one

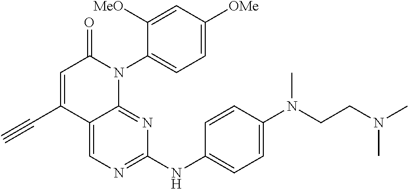 [SMILES: C#Cc1cc(=O)n(-c2ccc(OC)cc2OC)c2nc(Nc3ccc(N(C)CCN(C)C)cc3)ncc12]

General procedure 15 was applied to 8-(2,4-dimethoxyphenyl)-2-[(4-{[2-(dimethylamino)ethyl](methyl) amino}phenyl)amino]-5-[2-(triisopropylsilyl)ethynyl]pyrido[2,3-d]pyrimidin-7-one (120 mg, 0.183 mmol) and potassium fluoride (106 mg, 1.83 mmol) in methanol (2.0 mL). The resulting mixture was stirred for 1 h at 70° C. The residue was purified by reverse phase flash column chromatography eluting with acetonitrile (20-80%) in water to yield the title compound as a reddish solid (41.4 mg, 45%).

(ES, m/z): [M+H]⁺=499.3.

¹H NMR (300 MHz, DMSO-d₆) δ 9.95 (s, 1H), 8.74 (s, 1H), 8.19 (d, J=1.6 Hz, 1H), 7.17 (d, J=8.6 Hz, 3H), 6.82 (d, J=2.5 Hz, 1H), 6.71 (dd, J=8.6, 2.6 Hz, 1H), 6.57 (s, 1H), 6.36 (d, J=8.6 Hz, 2H), 5.09 (s, 1H), 3.90 (s, 4H), 3.65 (s, 4H), 3.35 (t, J=7.2 Hz, 3H), 2.83 (s, 3H), 2.47-2.36 (m, 2H), 2.25 (d, J=2.0 Hz, 6H).

### Example 87

### Ethyl 2-(methylsulfanyl)-4-{[(1r,4r)-4-[(tert-butoxycarbonyl)amino]cyclohexyl]amino}pyrimidine-5-carboxylate

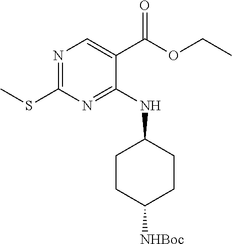 [SMILES: CCOC(=O)c1cnc(SC)nc1N[C@H]1CC[C@H](NC(=O)OC(C)(C)C)CC1]

General procedure 2 was applied to ethyl 4-chloro-2-(methylsulfanyl)pyrimidine-5-carboxylate (30.0 g, 129 mmol) with triethylamine (13.1 g, 129 mmol) and tert-butyl N-[(1r,4r)-4-aminocyclohexyl]carbamate (27.6 g, 129 mmol) in THF (500 mL) to yield the title compound as a white solid (45.0 g, 85%).

(ES, m/z): [M+H]⁺=511.5

### 2-(Methylsulfanyl)-4-{[(1r,4r)-4-[(tert-butoxycarbonyl)amino]cyclohexyl]amino}pyrimidine-5-carboxylic acid

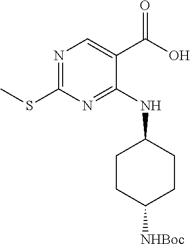 [SMILES: CSc1ncc(C(=O)O)c(N[C@H]2CC[C@H](NC(=O)OC(C)(C)C)CC2)n1]

General procedure 3 was applied to ethyl 2-(methylsulfanyl)-4-{[(1r,4r)-4-[(tert-butoxycarbonyl)amino]cyclohexyl]amino}pyrimidine-5-carboxylate (45.0 g, 110 mmol) with sodium hydroxide (8.77 g, 219 mmol) in THF (500 mL) and water (100 mL) to yield the title compound as an off-white solid (38.0 g, 91%).

(ES, m/z): [M+H]⁺=383.4

### tert-Butyl N-[(1r,4r)-4-{[5-(1,2,3-benzotriazole-1-carbonyl)-2-(methylsulfanyl)pyrimidin-4-yl]amino}cyclohexyl]carbamate

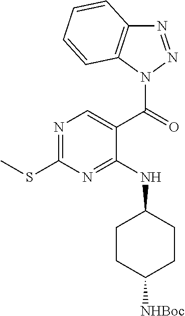 [SMILES: CSc1ncc(C(=O)n2nnc3ccccc32)c(N[C@H]2CC[C@H](NC(=O)OC(C)(C)C)CC2)n1]

General procedure 4 was applied to 2-(methylsulfanyl)-4-{[(1r,4r)-4-[(tert-butoxycarbonyl)amino]cyclohexyl]amino}pyrimidine-5-carboxylic acid (38.0 g, 99.3 mmol) with benzotriazole (11.8 g, 99.3 mmol) and EDCl (19.0 g, 99.3 mmol) in dichloromethane (300 mL). The crude material was diluted with methanol (300 mL) and the precipitated solids collected by filtration and washing with methanol (2×50 mL) to yield the title compound as a white solid (45.0 g, 94%).

(ES, m/z): [M+H]⁺=484.5

### Ethyl 3-[2-(methylsulfanyl)-4-{[(1r,4r)-4-[(tert-butoxycarbonyl)amino]cyclohexyl]amino}pyrimidin-5-yl]-3-oxopropanoate

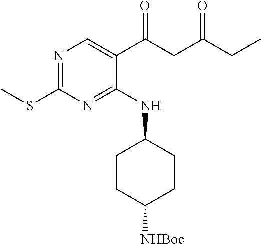 [SMILES: CCC(=O)CC(=O)c1cnc(SC)nc1N[C@H]1CC[C@H](NC(=O)OC(C)(C)C)CC1]

General procedure 5 was applied to tert-butyl N-[(1r,4r)-4-{[5-(1,2,3-benzotriazole-1-carbonyl)-2-(methylsulfanyl)pyrimidin-4-yl]amino}cyclohexyl]carbamate (20.0 g, 41.4 mmol) with ethyl acetate (10.1.0 mL, 103 mmol) and 1 M LiHMDS in THF (103.0 mL, 103 mmol) in THF (2×100 mL). The crude material was purified by flash column chromatography eluting with ethyl acetate (10%) in 40-60 petroleum ether to yield the title compound as an off-white solid (10.0 g, 53%).

(ES, m/z): [M+H]⁺=437.5

### tert-Butyl N-[(1r,4r)-4-[5-hydroxy-2-(methylsulfanyl)-7-oxopyrido[2,3-d]pyrimidin-8-yl]cyclohexyl]carbamate

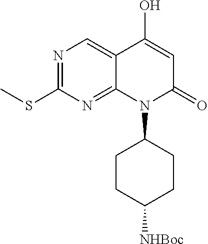 [SMILES: CSc1ncc2c(O)cc(=O)n([C@H]3CC[C@H](NC(=O)OC(C)(C)C)CC3)c2n1]

General procedure 6 was applied to ethyl 3-[2-(methylsulfanyl)-4-{[(1r,4r)-4-[(tert-butoxycarbonyl)amino]cyclohexyl]amino}pyrimidin-5-yl]-3-oxopropanoate (10.0 g, 22.1 mmol) and N,N-diisopropylethylamine (38.5 mL, 221 mmol), DBU (6.60 mL, 44.2 mmol) to yield the title compound as an off-white solid (6.00 g, 67%).

(ES, m/z): [M+H]⁺=407.5

### tert-Butyl N-[(1r,4r)-4-[2-(methylsulfanyl)-7-oxo-5-(trifluoromethanesulfonyloxy)pyrido[2,3-d]pyrimidin-8-yl]cyclohexyl]carbamate

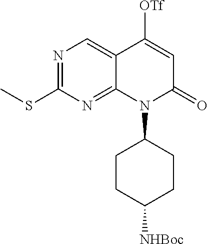 [SMILES: CSc1ncc2c(OS(=O)(=O)C(F)(F)F)cc(=O)n([C@H]3CC[C@H](NC(=O)OC(C)(C)C)CC3)c2n1]

General procedure 8 was applied to tert-butyl N-[(1r,4r)-4-[5-hydroxy-2-(methylsulfanyl)-7-oxopyrido[2,3-d]pyrimidin-8-yl]cyclohexyl]carbamate (6.00 g, 14.7 mmol) with triflic anhydride (5.00 g, 17.7 mmol) and triethylamine (3.58 g, 35.4 mmol) in dichloromethane (20 mL). The residue was purified by flash column chromatography eluting with ethyl acetate (8%) in 40-60 petroleum ether to afford the title compound as an off-white solid (4.00 g, 50%).

(ES, m/z): [M+H]⁺=539.5

### tert-Butyl N-[(1r,4r)-4-[2-(methylsulfanyl)-7-oxo-5-[2-(triisopropylsilyl)ethynyl]pyrido[2,3-d]pyrimidin-8-yl]cyclohexyl]carbamate

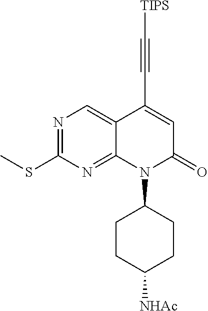

General procedure 9 was applied to tert-butyl N-[(1r,4r)-4-[2-(methylsulfanyl)-7-oxo-5-(trifluoromethanesulfonyloxy)pyrido[2,3-d]pyrimidin-8-yl]cyclohexyl]carbamate (4.00 g, 7.43 mmol) with triisopropylsilylacetylene (2.71 g, 14.8 mmol), bis(triphenylphsophine)palladium(II) dichloride (520 mg, 0.743 mmol) and copper(I) iodide (140 mg, 0.743 mmol) in DMF (20 mL) and N,N-diisopropylethylamine (1.92 g, 14.8 mmol). The residue was purified by flash column chromatography, eluting with ethyl acetate (8%) 40-60 petroleum ether to afford the title compound as a yellow-brown solid (3.00 g, 71%).

(ES, m/z): [M+H]⁺=429.6

### 2-(Methylsulfanyl)-8-[(1r,4r)-4-aminocyclohexyl]-5-[2-(triisopropylsilyl)ethynyl]pyrido[2,3-d]pyrimidin-7-one

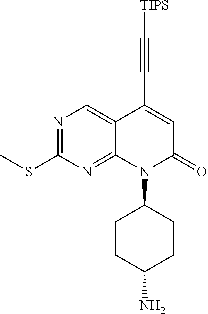

A solution of tert-butyl N-[(1r,4r)-4-[2-(methylsulfanyl)-7-oxo-5-[2-(triisopropylsilyl)ethynyl]pyrido[2,3-d]pyrimidin-8-yl]cyclohexyl]carbamate (800 mg, 1.40 mmol) in trifluoroacetic acid (5.0 mL) was stirred for 30 min at room temperature. The resulting mixture was concentrated under reduced pressure to yield the title compound as a yellow oil (700 mg, >100%).

(ES, m/z): [M+H]⁺=439.44

### N-[(1r,4r)-4-[2-(Methylsulfanyl)-7-oxo-5-[2-(triisopropylsilyl)ethynyl]pyrido[2,3-d]pyrimidin-8-yl]cyclohexyl]acetamide

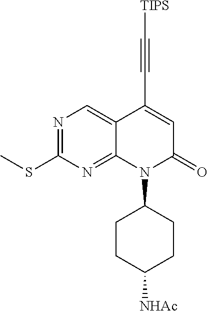

To a stirred solution of 2-(methylsulfanyl)-8-[(1r,4r)-4-aminocyclohexyl]-5-[2-(triisopropylsilyl)ethynyl]pyrido[2,3-d]pyrimidin-7-one (700 mg, 1.49 mmol) and triethylamine (301 mg, 2.97 mmol) in dichloromethane (10 mL) was added acetic anhydride (159 mg, 1.56 mmol) dropwise at 0° C. The resulting mixture was stirred for 30 min at room temperature, concentrated under reduced pressure and the crude residue was purified by reverse phase flash column chromatography eluting acetonitrile (20-100%) in water to yield the title compound as an off-white solid (640 mg, 84%).

(ES, m/z): [M+H]⁺=481.4

### N-[(1r,4r)-4-{2-Methanesulfonyl-7-oxo-5-[2-(triisopropylsilyl)ethynyl]pyrido[2,3-d]pyrimidin-8-yl}cyclohexyl]acetamide

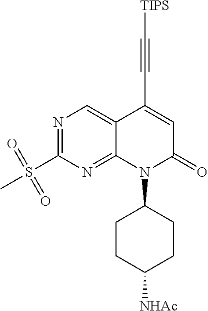

General procedure 10 was applied to N-[(1r,4r)-4-[2-(methylsulfanyl)-7-oxo-5-[2-(triisopropylsilyl)ethynyl]pyrido[2,3-d]pyrimidin-8-yl]cyclohexyl]acetamide (640 mg, 1.25 mmol) and m-CPBA (754 mg, 4.39 mmol) in dichloromethane (10 mL). The residue was purified by reverse phase flash column chromatography eluting with acetonitrile (20-100%) in water to yield the title compound as a white solid (550 mg, 81%).

(ES, m/z): [M+H]⁺=545.7.

### N-[(1r,4r)-4-(2-{[4-(4-Methylpiperazin-1-yl)phenyl]amino}-7-oxo-5-[2-(triisopropylsilyl)ethynyl]pyrido[2,3-d]pyrimidin-8-yl)cyclohexyl]acetamide

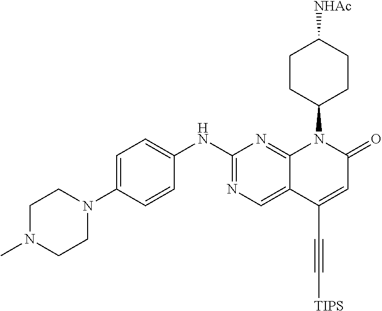

General procedure 13 was applied to N-[(1r,4r)-4-{2-methanesulfonyl-7-oxo-5-[2-(triisopropylsilyl)ethynyl]pyrido[2,3-d]pyrimidin-8-yl}cyclohexyl]acetamide (550 mg, 1.01 mmol) with trifluoroacetic acid (115 mg, 1.01 mmol) and 4-(4-methylpiperazin-1-yl)aniline (193 mg, 1.01 mmol) in 2-butanol (10 mL). The resulting mixture was stirred for 5 h at 110° C. The crude residue was purified by reverse phase flash column chromatography eluting with acetonitrile (10-50%) in water to yield the title compound as a light brown solid (420 mg, 63%).

(ES, m/z): [M+H]⁺=670.9.

### N-[(1r,4r)-4-(5-Ethynyl-2-{[4-(4-methylpiperazin-1-yl)phenyl]amino}-7-oxopyrido[2,3-d]pyrimidin-8-yl)cyclohexyl]acetamide

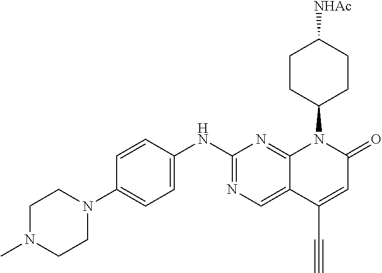 [SMILES: C#Cc1cc(=O)n([C@H]2CC[C@H](NC(C)=O)CC2)c2nc(Nc3ccc(N4CCN(C)CC4)cc3)ncc12]

General procedure 15 was applied to N-[(1r,4r)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]acetamide (150 mg, 0.229 mmol) and potassium fluoride (133 mg, 2.29 mmol) in THF (2.0 mL), methanol (1.0 mL) and water (0.30 mL). The resulting mixture was stirred for 30 min at 75° C. The crude residue was purified by reverse phase flash column chromatography eluting with acetonitrile (18-50%) in water to yield the title compound as a yellow solid (84.0 mg, 74%).

(ES, m/z): [M+H]⁺=500.5

¹H NMR (300 MHz, DMSO-d₆) δ 9.98 (d, J=45.8 Hz, 2H), 8.76 (s, 1H), 7.85 (d, J=7.7 Hz, 1H), 7.64 (d, J=8.6 Hz, 2H), 7.38-6.92 (m, 3H), 6.51 (s, 1H), 5.17 (d, J=28.7 Hz, 1H), 5.03 (s, 1H), 3.53 (d, J=11.7 Hz, 3H), 3.17 (s, 2H), 2.96 (t, J=12.4 Hz, 2H), 2.88 (s, 3H), 1.92 (d, J=12.3 Hz, 2H), 1.84 (s, 3H), 1.58 (d, J=11.7 Hz, 2H), 1.32 (q, J=12.6, 12.0 Hz, 2H).

### Example 88

### N-(4-(2-((2-Methoxyphenyl)amino)-7-oxo-5-((triisopropylsilyl)ethynyl)pyrido[2,3-d]pyrimidin-8(7H)-yl)phenyl)acrylamide

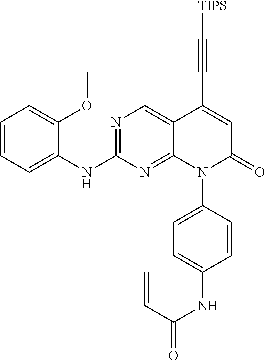

A solution of 8-(4-aminophenyl)-2-((2-methoxyphenyl)amino)-5-((triisopropylsilyl)ethynyl)pyrido[2,3-d]pyrimidin-7(8H)-one (210 mg, 0.390 mmol), triethylamine (27.0 μL, 0.195 mmol) and acryloyl chloride (31.6 μL, 0.390 mmol) in dichloromethane (7.0 mL) was stirred at room temperature for 2 h and concentrated under reduced pressure. The crude material was purified by flash column chromatography eluting with ethyl acetate (30-70%) in 40-60 petroleum ether to yield the title compound as a yellow solid (20.6 mg, 30.0 μmol, 8%).

(ES, m/z): [M+H]⁺=594.5.

### N-(4-(5-Ethynyl-2-((2-methoxyphenyl)amino)-7-oxopyrido[2,3-d]pyrimidin-8(7H)-yl)phenyl)acrylamide

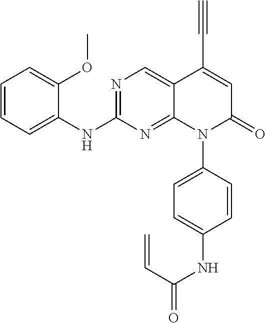 [SMILES: C#Cc1cc(=O)n(-c2ccc(NC(=O)C=C)cc2)c2nc(Nc3ccccc3OC)ncc12]

General procedure 15 was applied to N-(4-(2-((2-methoxyphenyl)amino)-7-oxo-5-((triisopropylsilyl)ethynyl)pyrido[2,3-d]pyrimidin-8(7H)-yl)phenyl)acrylamide (20.0 mg, 30.0 μmol) and potassium fluoride (35.0 mg, 0.600 mmol) in DMF (0.60 mL). The reaction was stirred at room temperature for 6 h. The crude material was purified by flash column chromatography eluting with ethyl acetate (50-60%) in 40-60 petroleum ether to yield the title compound as a yellow solid (5.00 mg, 11.4 μmol, 38%).

(ES, m/z): [M+H]⁺=438.2.

¹H-NMR (500 MHz, DMSO-d₆) δ 10.38 (s, 1H), 8.82 (s, 1H), 8.40 (s, 1H), 7.84 (d, J=8.7 Hz, 2H), 7.47 (d, J=8.0 Hz, 1H), 7.30 (d, J=8.8 Hz, 2H), 6.95 (d, J=7.9 Hz, 1H), 6.70 (s, 1H), 6.52 (dd, J=17.0, 10.3 Hz, 1H), 6.39-6.29 (m, 1H), 5.86-5.75 (m, 1H), 5.12 (s, 1H), 3.81 (s, 3H).

### Example 89

### N-[(1r,4r)-4-[2-(Methylsulfanyl)-7-oxo-5-[2-(triisopropylsilyl)ethynyl]pyrido[2,3-d]pyrimidin-8-yl]cyclohexyl]pyridazine-4-carboxamide

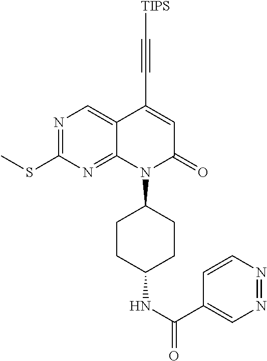

To a solution of 2-(methylsulfanyl)-8-[(1r,4r)-4-aminocyclohexyl]-5-[2-(triisopropylsilyl)ethynyl]pyrido[2,3-d]pyrimidin-7-one (800 mg, 1.70 mmol, 1.0 eq.) and triethylamine (258 mg, 2.55 mmol, 1.5 eq.) in dichloromethane (8.0 mL) was added HATU (678 mg, 1.78 mmol, 1.05 eq.) and pyridazine-4-carboxylic acid (221 mg, 1.78 mmol, 1.05 eq.) dropwise at 0° C. The reaction mixture was stirred at room temperature for 2 h and applied onto a reverse phase column, eluting with acetonitrile (10-100%) in water (0.1% formic acid). The desired fractions were collected and concentrated under reduced pressure to remove most of the acetonitrile and lyophilized to yield the title compound as a brown solid (650 mg, 60%).

(ES, m/z): [M+H]⁺=577.2.

### N-[(1r,4r)-4-{2-Methanesulfonyl-7-oxo-5-[2-(triisopropylsilyl)ethynyl]pyrido[2,3-d]pyrimidin-8-yl}cyclohexyl]pyridazine-4-carboxamide

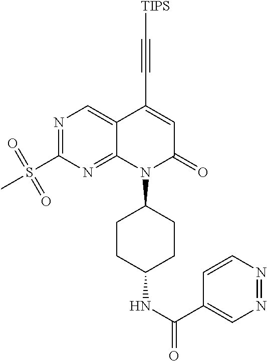

General procedure 10 was applied to N-[(1r,4r)-4-[2-(methylsulfanyl)-7-oxo-5-[2-(triisopropylsilyl)ethynyl]pyrido[2,3-d]pyrimidin-8-yl]cyclohexyl]pyridazine-4-carboxamide (650 mg, 1.13 mmol), m-CPBA (583 mg, 3.38 mmol) and dichloromethane (7.0 mL) to yield the title compound as a brown solid (550 mg, 72%).

(ES, m/z): [M+H]⁺=609.2.

### N-[(1r,4r)-4-(2-{[4-(4-Methylpiperazin-1-yl)phenyl]amino}-7-oxo-5-[2-(triisopropylsilyl)ethynyl]pyrido[2,3-d]pyrimidin-8-yl)cyclohexyl]pyridazine-4-carboxamide

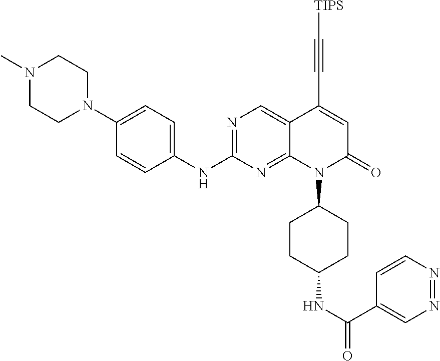

General procedure 13 was applied to N-[(1r,4r)-4-{2-methanesulfonyl-7-oxo-5-[2-(triisopropylsilyl)ethynyl]pyrido[2,3-d]pyrimidin-8-yl}cyclohexyl]pyridazine-4-carboxamide (550 mg, 0.903 mmol), 4-(4-methylpiperazin-1-yl)aniline (259 mg, 1.35 mmol), trifluoroacetic acid (257 mg, 2.26 mmol) and 2-butanol (10 mL). The reaction mixture was stirred at 110° C. for 24 h. The reaction mixture was purified by reverse phase column, eluting with acetonitrile (10-100%) in water (0.1% trifluoroacetic acid) to yield the title compound as an orange solid (300 mg, 46%).

(ES, m/z): [M+H]⁺=720.4.

### N-[(1r,4r)-4-(5-Ethynyl-2-{[4-(4-methylpiperazin-1-yl)phenyl]amino}-7-oxopyrido[2,3-d]pyrimidin-8-yl)cyclohexyl]pyridazine-4-carboxamide

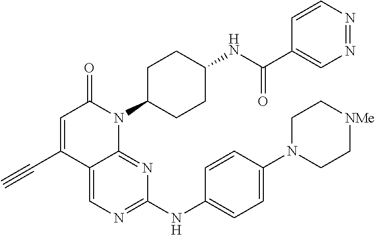 [SMILES: C#Cc1cc(=O)n([C@H]2CC[C@H](NC(=O)c3ccnnc3)CC2)c2nc(Nc3ccc(N4CCN(C)CC4)cc3)ncc12]

General procedure 15 was applied to N-[(1r,4r)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]pyridazine-4-carboxamide (300 mg, 0.417 mmol) and potassium fluoride (242 mg, 4.17 mmol) in THF (3.0 mL), methanol (1.5 mL,) and water (0.10 mL). The reaction mixture was stirred at room temperature for 2 h and purified by reverse phase column eluting with acetonitrile (10-100%) in water (0.1% formic acid). The desired fractions were collected and concentrated under reduced pressure to remove most of the acetonitrile then lyophilized to yield the title compound as an orange solid (24.0 mg, 9%).

(ES, m/z): [M+H]⁺=564.5

¹H NMR (400 MHz, DMSO-d₆) δ 10.09 (s, 1H), 9.83 (s, 1H), 9.59 (s, 1H), 9.47 (d, J=5.3 Hz, 1H), 8.93 (d, J=7.6 Hz, 1H), 8.77 (s, 1H), 8.04 (d, J=3.1 Hz, 1H), 7.67 (s, 2H), 7.33-6.97 (m, 5H), 6.53 (s, 1H), 5.23 (s, 1H), 5.05 (s, 1H), 3.84 (d, J=47.3 Hz, 3H), 3.53 (d, J=12.7 Hz, 3H), 3.18 (s, 3H), 2.98 (t, J=11.9 Hz, 3H), 2.87 (s, 3H), 2.04 (s, 2H), 1.74-1.47 (m, 4H).

### Example 90

### tert-Butyl ((1s,4s)-4-(2-(methylsulfonyl)-7-oxo-5-((triisopropylsilyl)ethynyl)pyrido[2,3-d]pyrimidin-8(7H)-yl)cyclohexyl)carbamate

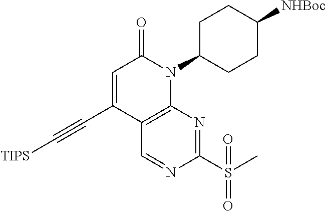

General procedure 10 was applied to tert-butyl ((1s,4s)-4-(2-(methylthio)-7-oxo-5-((triisopropylsilyl)ethynyl)pyrido[2,3-d]pyrimidin-8(7H)-yl)cyclohexyl)carbamate (3.00 g, 5.26 mmol) and m-CPBA (1.99 g, 11.6 mmol) in dichloromethane (30 mL) to yield the title compound as a yellow solid (1.60 g, 30%).

### tert-Butyl N-[(1s,4s)-4-{2-[(2-methoxyphenyl)amino]-7-oxo-5-[2-(triisopropylsilyl)ethynyl]pyrido[2,3-d]pyrimidin-8-yl}cyclohexyl]carbamate

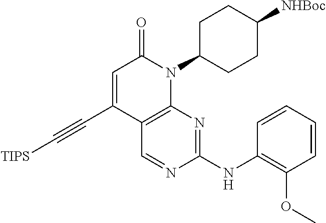

General procedure 13 was applied to tert-butyl N-[(1s,4s)-4-{2-methanesulfonyl-7-oxo-5-[2-(triisopropylsilyl) ethynyl]pyrido[2,3-d]pyrimidin-8-yl}cyclohexyl]carbamate (1.60 g, 2.65 mmol) with trifluoroacetic acid (1.21 g, 10.6 mmol) and o-anisidine (980 mg, 7.96 mmol) in 2-butanol (16 mL). The resulting mixture was stirred for 16 h at 110° C. and the crude mixture was purified by reverse phase flash column chromatography eluting with acetonitrile (40-60%) in water (0.1% formic acid) to afford the title compound as a yellow solid (900 mg, 53%).

### 2-[(2-Methoxyphenyl)amino]-8-[(1s,4s)-4-aminocyclohexyl]-5-[2-(triisopropylsilyl)ethynyl]pyrido[2,3-d]pyrimidin-7-one

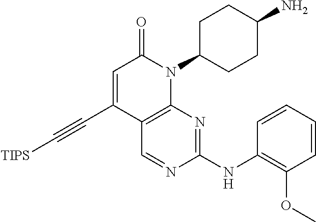

A solution of tert-butyl N-[(1s,4s)-4-{2-[(2-methoxyphenyl)amino]-7-oxo-5-[2-(triisopropylsilyl) ethynyl]pyrido[2,3-d]pyrimidin-8-yl}cyclohexyl]carbamate (600 mg, 0.929 mmol, 1.0 eq.) in 4M hydrogen chloride in 1,4-dioxane (6.00 mL) was stirred for 2 h at room temperature. The mixture was concentrated under reduced pressure to afford the title compound as a yellow solid (500 mg, 98%).

### 2-(Dimethylamino)-N-[(1s,4s)-4-{2-[(2-methoxyphenyl)amino]-7-oxo-5-[2-(triisopropylsilyl)ethynyl]pyrido[2,3-d]pyrimidin-8-yl}cyclohexyl]acetamide

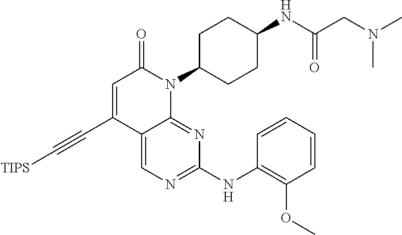

To a stirred mixture of 2-[(2-methoxyphenyl)amino]-8-[(1s,4s)-4-aminocyclohexyl]-5-[2-(triisopropylsilyl)ethynyl]pyrido[2,3-d]pyrimidin-7-one (460 mg, 0.843 mmol, 1.0 eq.) and N,N-dimethylglycine hydrochloride (129 mg, 0.927 mmol, 1.1 eq.) in DMF (5.0 mL) was added HOBT (125 mg, 0.927 mmol, 1.1 eq.), EDCl (323 mg, 1.69 mmol, 2.0 eq.) and triethylamine (256 mg, 2.53 mmol, 3.0 eq.). The resulting mixture was stirred for 2 h at room temperature before being quenched with water (10 mL) and extracted with ethyl acetate (3×10 mL). The combined organic layers were washed with brine (3×15 mL), dried (Na₂SO₄) and concentrated under reduced pressure. The crude residue was purified by flash column chromatography eluting with ethyl acetate (25%) in 40-60 petroleum ether to afford the title compound as a brown solid (400 mg, 75%).

### 2-(Dimethylamino)-N-[(1s,4s)-4-{5-ethynyl-2-[(2-methoxyphenyl) amino]-7-oxopyrido[2,3-d]pyrimidin-8-yl}cyclohexyl]acetamide

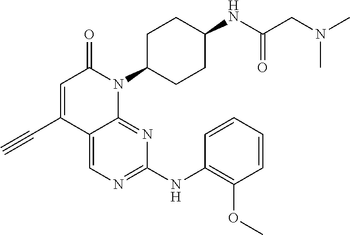 [SMILES: C#Cc1cc(=O)n([C@H]2CC[C@@H](NC(=O)CN(C)C)CC2)c2nc(Nc3ccccc3OC)ncc12]

General procedure 15 was applied to 2-(dimethylamino)-N-[(1s,4s)-4-{2-[(2-methoxyphenyl)amino]-7-oxo-5-[2-(triisopropylsilyl)ethynyl]pyrido[2,3-d]pyrimidin-8-yl}cyclohexyl]acetamide (100 mg, 0.159 mmol) and potassium fluoride (92.1 mg, 1.59 mmol) in DMF (2.0 mL), THF (2.0 mL) and water (0.40 mL). The resulting mixture was stirred for 1 h at 60° C. and purified by reverse phase flash column chromatography eluting with acetonitrile (20-40%) in water (0.1% formic acid) to afford the title compound as a yellow-green solid (70.0 mg, 91%).

(ES, m/z): [M+H]⁺=475

¹H-NMR (300 MHz, DMSO-d₆) δ 8.94 (s, 1H), 8.76 (s, 1H), 7.86 (d, J=7.6 Hz, 1H), 7.62 (d, J=7.3 Hz, 1H), 7.22-7.08 (m, 2H), 7.01 (t, J=7.4 Hz, 1H), 6.50 (s, 1H), 5.30 (s, 1H), 5.04 (s, 1H), 3.98 (s, 1H), 3.84 (s, 3H), 2.94 (s, 2H), 2.66 (d, J=13.9 Hz, 2H), 2.30 (s, 6H), 1.88 (d, J=13.2 Hz, 2H), 1.46 (d, J=13.6 Hz, 4H).

### Example 91

### tert-Butyl N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl) cyclohexyl]carbamate

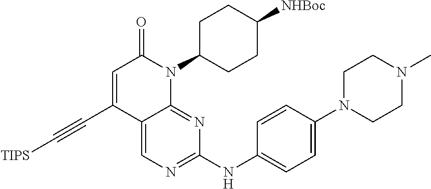

General procedure 13 was applied to tert-butyl N-[(1s,4s)-4-{2-methanesulfonyl-7-oxo-5-[2-(triisopropylsilyl) ethynyl]pyrido[2,3-d]pyrimidin-8-yl}cyclohexyl]carbamate (800 mg, 0.663 mmol) with trifluoroacetic acid (151 mg, 1.33 mmol) and 4-(4-methylpiperazin-1-yl)aniline (190 mg, 0.995 mmol) in 2-butanol (8.0 mL). The resulting mixture was stirred for 16 h at 100° C. and purified by reverse phase flash column chromatography eluting with acetonitrile (40-60%) in water (0.1% formic acid) to afford the title compound as a yellow solid (300 mg, 63%).

### 2-{[4-(4-Methylpiperazin-1-yl)phenyl]amino}-8-[(1s,4s)-4-aminocyclohexyl]-5-[2-(triisopropylsilyl)ethynyl]pyrido[2,3-d]pyrimidin-7-one

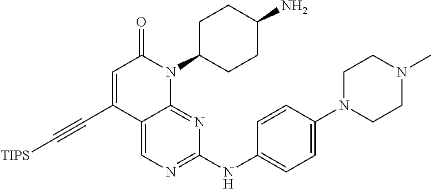

A solution of tert-butyl N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]carbamate (260 mg, 0.36 mmol, 1 eq.) in 4M hydrogen chloride in 1,4-dioxane (2.6 mL) was stirred for 2 h at room temperature. The reaction mixture was concentrated under reduced pressure to afford the title compound as a red solid (270 mg, >100%).

### 2-(1H-Pyrazol-4-yl)-N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]acetamide

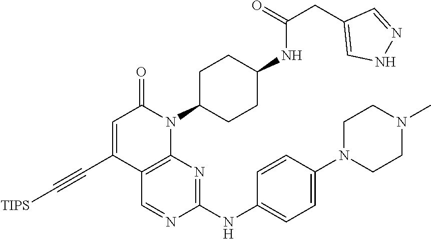

To a stirred mixture of 2-{[4-(4-methylpiperazin-1-yl)phenyl]amino}-8-[(1s,4s)-4-aminocyclohexyl]-5-[2-(triisopropylsilyl)ethynyl]pyrido[2,3-d]pyrimidin-7-one (270 mg, 0.440 mmol, 1 eq.) and 1H-pyrazol-4-yl acetic acid (61.0 mg, 0.48 mmol, 1.1 eq.) in DMF (1.0 mL) was added HOBT (65.4 mg, 0.48 mmol, 1.1 eq.), EDCl (169 mg, 0.880 mmol, 2.0 eq.) and triethylamine (133 mg, 1.32 mmol, 3.0 eq.). The resulting mixture was stirred for 3 h at room temperature and purified by reverse phase flash column chromatography eluting with acetonitrile (30-40%) in water (0.1% formic acid) to afford the title compound as a yellow solid (150 mg, 47%).

### 2-(1H-Pyrazol-4-yl)-N-[(1s,4s)-4-(5-ethynyl-2-{[4-(4-methylpiperazin-1-yl)phenyl]amino}-7-oxopyrido[2,3-d]pyrimidin-8-yl)cyclohexyl]acetamide

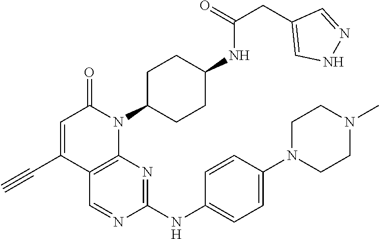 [SMILES: C#Cc1cc(=O)n([C@H]2CC[C@@H](NC(=O)Cc3cn[nH]c3)CC2)c2nc(Nc3ccc(N4CCN(C)CC4)cc3)ncc12]

General procedure 13 was applied to 2-(1H-pyrazol-4-yl)-N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]acetamide (150 mg, 0.208 mmol) and potassium fluoride (121 mg, 2.08 mmol) in DMF (2.0 mL), THF (2.0 mL) and water (0.50 mL). The resulting mixture was stirred for 1 h at 60° C. before being purified by reverse phase flash column chromatography eluting with acetonitrile (40-45%) in water (0.1% formic acid) to afford the title compound as a yellow solid (32 mg, 27%).

(ES, m/z): [M+H]⁺=566

¹H-NMR (300 MHz, DMSO-d₆) δ 8.75 (s, 1H), 8.17 (s, 1H), 7.83 (s, 1H), 7.58 (d, J=8.7 Hz, 2H), 7.50 (s, 2H), 6.95 (d, J=9.0 Hz, 2H), 6.47 (s, 1H), 5.34 (s, 1H), 5.03 (s, 1H), 3.89 (s, 1H), 3.10 (d, J=5.3 Hz, 4H), 2.73 (s, 2H), 2.47 (s, 4H), 2.24 (s, 3H), 1.93 (d, J=13.1 Hz, 2H), 1.54 (s, 2H), 1.43 (d, J=12.3 Hz, 2H).

### Example 92

### Methyl 3-{[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]carbamoyl}propanoate

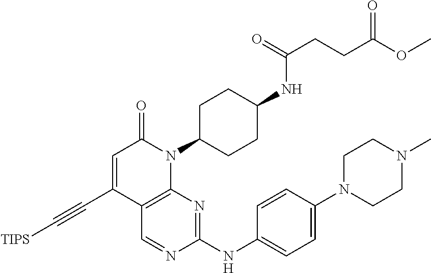

A solution of 2-{[4-(4-methylpiperazin-1-yl)phenyl]amino}-8-[(1s,4s)-4-aminocyclohexyl]-5-[2-(triisopropylsilyl)ethynyl]pyrido[2,3-d]pyrimidin-7-one (500 mg, 0.814 mmol, 1.0 eq.), 4-methoxy-4-oxobutanoic acid (118 mg, 0.895 mmol, 1.1 eq.), HOBT (121 mg, 0.895 mmol, 1.1 eq.), EDCl (312 mg, 1.63 mmol, 2.0 eq.) and triethylamine (247 mg, 2.44 mmol, 3.0 eq.) in dichloromethane (5.0 mL) was stirred for 2 h at 30° C. The reaction mixture was purified by reverse phase flash column chromatography eluting with acetonitrile (20-100%) in water (0.1% formic acid) and the collected fractions were concentrated under reduced pressure to remove most of the acetonitrile. The solution was acidified to pH 8 using a saturated aqueous sodium hydrogen carbonate solution and extracted with ethyl acetate (3×200 mL). The combined organic fractions were washed with brine (2×200 mL), dried (MgSO₄) and concentrated under reduced pressure to yield the title compound as an orange solid (450 mg, 76%).

(ES, m/z): [M+H]⁺=713.

### Methyl 4-(((1s,4s)-4-(5-ethynyl-2-((4-(4-methylpiperazin-1-yl)phenyl)amino)-7-oxopyrido[2,3-d]pyrimidin-8(7H)-yl)cyclohexyl)amino)-4-oxobutanoate

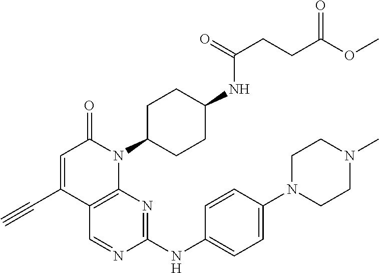 [SMILES: C#Cc1cc(=O)n([C@H]2CC[C@@H](NC(=O)CCC(=O)OC)CC2)c2nc(Nc3ccc(N4CCN(C)CC4)cc3)ncc12]

A solution of methyl 3-{[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]carbamoyl}propanoate (250 mg, 0.343 mmol, 1.0 eq.) and lithium hydroxide monohydrate (28.8 mg, 0.686 mmol, 2.0 eq.) in THF (2.5 mL) and water (0.50 mL) was stirred for 4 h at 30° C. The reaction mixture was purified by reverse phase flash column chromatography eluting with acetonitrile (20-100%) in water (0.1% formic acid). The desired fractions were collected and concentrated under reduced pressure to remove most of the acetonitrile then lyophilized to yield the title compound as an orange solid (50.9 mg, 27%).

(ES, m/z): [M+H]⁺=558

¹H-NMR (300 MHz, DMSO-d₆) δ 10.09 (s, 1H), 8.74 (s, 1H), 7.85-7.73 (m, 1H), 7.59 (d, J=8.5 Hz, 2H), 6.94 (d, J=8.6 Hz, 2H), 6.45 (s, 1H), 5.30 (s, 1H), 5.02 (s, 1H), 3.91 (s, 2H), 3.11 (t, J=5.0 Hz, 4H), 2.85-2.73 (m, 2H), 2.51 (s, 6H), 2.23 (s, 3H), 1.90-1.82 (m, 2H), 1.55-1.53 (m, 2H), 1.41 (d, J=12.2 Hz, 2H).

### Example 93

### N,N-Dimethyl-N′-[(1s,4s)-4-[2-(methylsulfanyl)-7-oxo-5-[2-(triisopropylsilyl)ethynyl]pyrido[2,3-d]pyrimidin-8-yl]cyclohexyl]succinamide

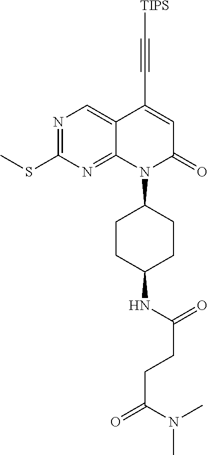

A solution of 2-(methylsulfanyl)-8-[(1s,4s)-4-aminocyclohexyl]-5-[2-(triisopropylsilyl)ethynyl]pyrido[2,3-d]pyrimidin-7-one (1.00 g, 2.12 mmol, 1.0 eq.), 3-(dimethylcarbamoyl)propanoic acid (339 mg, 2.34 mmol, 1.1 eq.), HOBT (316 mg, 2.34 mmol, 1.1 eq.), EDCl (814 mg, 4.25 mmol, 2.0 eq.) and triethylamine (645 mg, 6.37 mmol, 3.0 eq.) in dichloromethane (10 mL) was stirred for 2 h at 30° C. The reaction mixture purified by reverse phase column chromatography eluting with acetonitrile (20-100%) in water (0.1% formic acid). The desired fractions were collected and concentrated under reduced pressure to remove most of the acetonitrile. The mixture was basified to pH 8 with a saturated aqueous sodium hydrogen carbonate solution and extracted with ethyl acetate (3×200 mL). The organic layers were washed with brine (2×200 mL), dried (MgSO₄) and concentrated under reduced pressure to yield the title compound as a yellow solid (800 mg, 63%).

(ES, m/z): [M+H]⁺=598.

### N,N-Dimethyl-N′-[(1s,4s)-4-{2-methanesulfonyl-7-oxo-5-[2-(triisopropylsilyl)ethynyl]pyrido[2,3-d]pyrimidin-8-yl}cyclohexyl]succinamide

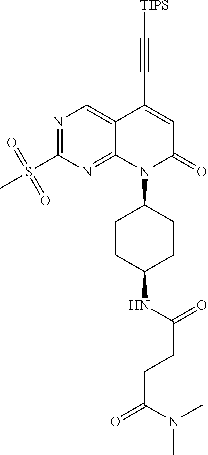

General procedure 10 was applied to N,N-dimethyl-N′[(1s,4s)-4-[2-(methylsulfanyl)-7-oxo-5-[2-(triisopropylsilyl)ethynyl]pyrido[2,3-d]pyrimidin-8-yl]cyclohexyl]succinamide (800 mg, 1.34 mmol), m-CPBA (577 mg, 3.35 mmol) in dichloromethane (8.0 mL) to yield the title compound as a yellow solid (800 mg, 95%).

(ES, m/z): [M+H]⁺=630.

### N,N-Dimethyl-N′-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]succinamide

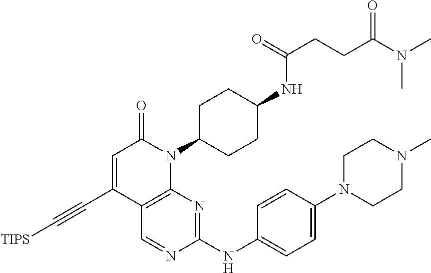

General procedure 13 was applied to N,N-dimethyl-N′-[(1s,4s)-4-{2-methanesulfonyl-7-oxo-5-[2-(triisopropylsilyl)ethynyl]pyrido[2,3-d]pyrimidin-8-yl}cyclohexyl]succinamide (800 mg, 1.27 mmol) with trifluoroacetic acid (289 mg, 2.54 mmol) and 4-(4-methylpiperazin-1-yl)aniline (291 mg, 1.52 mmol) in 2-butanol (8.0 mL). The resulting solution was stirred for 3 h at 100° C., diluted with methanol (10 mL) and purified by reverse phase column chromatography, eluting with acetonitrile (70-100%) in water (0.1% trifluoroacetic acid). The mixture was basified to pH 8 with a saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (550 mg, 58%).

(ES, m/z): [M+H]⁺=741.

### N,N-Dimethyl-N′-[(1s,4s)-4-(5-ethynyl-2-{[4-(4-methylpiperazin-1-yl)phenyl]amino}-7-oxopyrido[2,3-d]pyrimidin-8-yl)cyclohexyl]succinamide

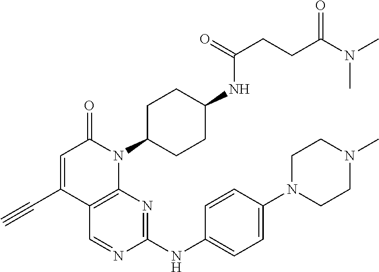 [SMILES: C#Cc1cc(=O)n([C@H]2CC[C@@H](NC(=O)CCC(=O)N(C)C)CC2)c2nc(Nc3ccc(N4CCN(C)CC4)cc3)ncc12]

General procedure 15 was applied to N,N-dimethyl-N′-[(1s,4s)-4-(2-{[4-(4-methylpiperazin-1-yl) phenyl]amino}-7-oxo-5-[2-(triisopropylsilyl)ethynyl]pyrido[2,3-d]pyrimidin-8-yl)cyclohexyl]succinamide (250 mg, 0.337 mmol) and potassium fluoride (196 mg, 3.37 mmol) in THF (2.5 mL), DMF (2.5 mL) and water (0.50 mL). The resulting solution was stirred for 2 h at 70° C. and purified by reverse phase flash column chromatography, eluting with acetonitrile (20-100%) in water (0.1% formic acid). The desired fractions were collected and concentrated under reduced pressure to remove most of the acetonitrile then lyophilized to yield the title compound as an orange solid (110 mg, 56%).

(ES, m/z): [M+H]⁺=585

¹H-NMR (300 MHz, DMSO-d₆) δ 10.09 (s, 1H), 8.74 (s, 1H), 7.85-7.73 (m, 1H), 7.59 (d, J=8.5 Hz, 2H), 6.94 (d, J=8.6 Hz, 2H), 6.45 (s, 1H), 5.30 (s, 1H), 5.02 (s, 1H), 3.91 (s, 1H), 3.11 (t, J=5.0 Hz, 4H), 2.95 (s, 3H), 2.85-2.78 (m, 5H), 2.60-2.55 (m, 1H), 2.51 (s, 6H), 2.23 (s, 3H), 1.90-1.82 (m, 2H), 1.55-1.53 (m, 2H), 1.41 (d, J=12.2 Hz, 2H).

### Example 94

### N-Methyl-N′-[(1s,4s)-4-[2-(methylsulfanyl)-7-oxo-5-[2-(triisopropylsilyl)ethynyl]pyrido[2,3-d]pyrimidin-8-yl]cyclohexyl]propanediamide

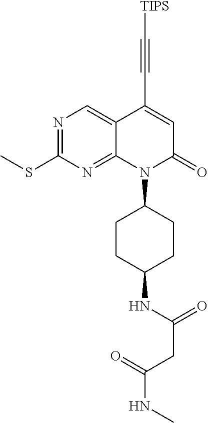

To a stirred solution of 2-(methylsulfanyl)-8-[(1s,4s)-4-aminocyclohexyl]-5-[2-(triisopropylsilyl) ethynyl]pyrido[2,3-d]pyrimidin-7-one (1.00 g, 2.12 mmol, 1.0 eq.) and 2-(methylcarbamoyl)acetic acid (274 mg, 2.34 mmol, 1.1 eq.) in DMF (10 mL) were added HOBT (316 mg, 2.34 mmol, 1.1 eq.) and EDCl (814 mg, 4.25 mmol, 2.0 eq.) and triethylamine (645 mg, 6.37 mmol, 3.0 eq.). The resulting mixture was stirred for 2 h at room temperature and was purified by reverse phase flash column chromatography, eluting with acetonitrile (40-65%) in water (0.4% formic acid) to afford the title compound as a light yellow solid (540 mg, 45%).

(ES, m/z): [M+H]⁺=570.

### N-Methyl-N′-[(1s,4s)-4-{2-methanesulfonyl-7-oxo-5-[2-(triisopropylsilyl)ethynyl]pyrido[2,3-d]pyrimidin-8-yl}cyclohexyl]propanediamide

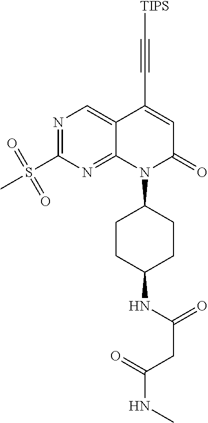

General procedure 10 was applied to N-methyl-N′-[(1s,4s)-4-[2-(methylsulfanyl)-7-oxo-5-[2-(triisopropylsilyl) ethynyl]pyrido[2,3-d]pyrimidin-8-yl]cyclohexyl]propanediamide (540 mg, 0.948 mmol) with m-CPBA (327 mg, 1.89 mmol) in dichloromethane (5.0 mL) to afford the title compound as a yellow solid (800 mg, >100%).

(ES, m/z): [M+H]⁺=602

### N-Methyl-N′-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanediamide

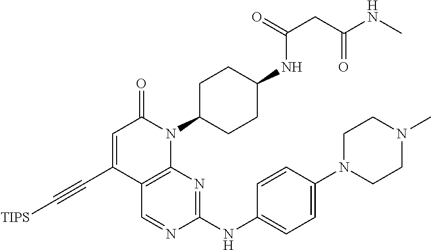

General procedure 13 was applied to N-methyl-N′-[(1s,4s)-4-{2-methanesulfonyl-7-oxo-5-[2-(triisopropylsilyl) ethynyl]pyrido[2,3-d]pyrimidin-8-yl}cyclohexyl]propanediamide (800 mg, 1.33 mmol) with trifluoroacetic acid (303 mg, 2.66 mmol) and 4-(4-methylpiperazin-1-yl)aniline (381 mg, 1.99 mmol) in 2-butanol (8.0 mL). The resulting mixture was stirred for 2 h at 100° C. and purified by reverse phase flash column chromatography eluting with acetonitrile (40-65%) in water (0.4% formic acid) to afford the title compound as a yellow solid (350 mg, 37%).

(ES, m/z): [M+H]⁺=713

### N-Methyl-N′-[(1s,4s)-4-(5-ethynyl-2-{[4-(4-methylpiperazin-1-yl)phenyl]amino}-7-oxopyrido[2,3-d]pyrimidin-8-yl)cyclohexyl]propanediamide

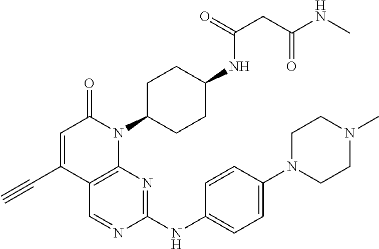 [SMILES: C#Cc1cc(=O)n([C@H]2CC[C@@H](NC(=O)CC(=O)NC)CC2)c2nc(Nc3ccc(N4CCN(C)CC4)cc3)ncc12]

General procedure 15 was applied to N-methyl-N′-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanediamide (250 mg, 0.351 mmol) with potassium fluoride (204 mg, 3.51 mmol) in DMF (3.0 mL) and THF (3.0 mL). The resulting mixture was stirred for 1 h at 60° C. and purified by reverse phase flash column chromatography, eluting acetonitrile (10-50%) in water (0.4% formic acid) to afford the title compound as an orange solid (67.0 mg, 34%).

(ES, m/z): [M+H]⁺=557

¹H-NMR (300 MHz, DMSO-d₆) δ 10.08 (s, 1H), 8.75 (s, 1H), 8.05 (d, J=56.0 Hz, 2H), 7.56 (s, 2H), 6.95 (d, J=8.7 Hz, 2H), 6.47 (s, 1H), 5.30 (s, 1H), 5.02 (s, 1H), 3.97 (s, 1H), 3.25-2.95 (m, 8H), 2.57 (s, 7H), 2.30 (s, 3H), 1.87 (s, 2H), 1.58 (s, 2H), 1.45 (d, J=12.1 Hz, 2H).

### Example 95

### N,N-Dimethyl-N′-[(1s,4s)-4-[2-(methylsulfanyl)-7-oxo-5-[2-(triisopropylsilyl)ethynyl]pyrido[2,3-d]pyrimidin-8-yl]cyclohexyl]propanediamide

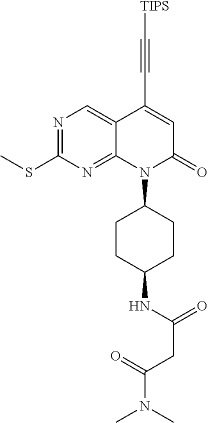

To a stirred mixture of 2-(methylsulfanyl)-8-[(1s,4s)-4-aminocyclohexyl]-5-[2-(triisopropylsilyl) ethynyl]pyrido[2,3-d]pyrimidin-7-one (1.60 g, 3.40 mmol, 1.0 eq.) and 2-(methylcarbamoyl)acetic acid (274 mg, 2.34 mmol, 1.1 eq.) in DMF (16 mL) were added HOBT (510 mg, 3.74 mmol, 1.1 eq.) and EDCl (1.30 g, 6.80 mmol, 2.0 eq.) and triethylamine (1.03 g, 10.2 mmol, 3.0 eq.). The resulting mixture was stirred for 2 h at 25° C. under a nitrogen atmosphere. The reaction mixture was purified by reverse phase flash column chromatography, eluting with acetonitrile (40-65%) in water (0.4% formic acid) to afford the title compound as a light yellow solid (640 mg, 32%).

(ES, m/z): [M+H]⁺=584.

### N,N-Dimethyl-N′-[(1s,4s)-4-{2-methanesulfonyl-7-oxo-5-[2-(triisopropylsilyl)ethynyl]pyrido[2,3-d]pyrimidin-8-yl}cyclohexyl]propanediamide

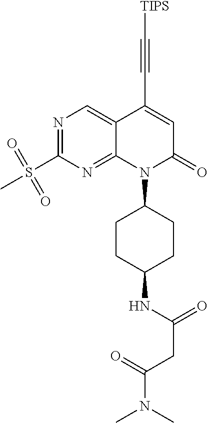

General procedure 10 was applied to N-methyl-N′[(1s,4s)-4-[2-(methylsulfanyl)-7-oxo-5-[2-(triisopropylsilyl) ethynyl]pyrido[2,3-d]pyrimidin-8-yl]cyclohexyl]propanediamide (540 mg, 0.948 mmol) with m-CPBA (416 mg, 2.41 mmol) in dichloromethane (7.0 mL) to afford the title compound as a yellow solid (600 mg, 89%).

(ES, m/z): [M+H]⁺=616.

### N,N-Dimethyl-N′-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanediamide

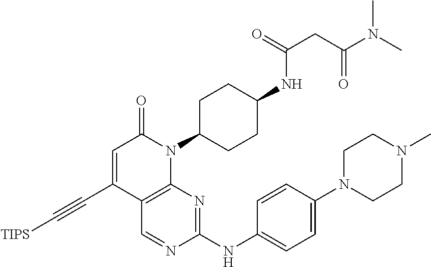

General procedure 13 was applied to N,N-dimethyl-N′-[(1s,4s)-4-{2-methanesulfonyl-7-oxo-5-[2-(triisopropylsilyl) ethynyl]pyrido[2,3-d]pyrimidin-8-yl}cyclohexyl]propanediamide (600 mg, 0.974 mmol) and 4-(4-methylpiperazin-1-yl)aniline (279 mg, 1.46 mmol), trifluoroacetic acid (222 mg, 1.95 mmol) in 2-butanol (6.0 mL). The resulting mixture was stirred for 2 h at 100° C. and purified by reverse phase flash column chromatography, eluting with acetonitrile (40-65%) in water (0.4% formic acid) to afford the title compound as a yellow solid (420 mg, 59%).

(ES, m/z): [M+H]⁺=727.

### N,N-Dimethyl-N′-[(1s,4s)-4-(5-ethynyl-2-{[4-(4-methylpiperazin-1-yl)phenyl]amino}-7-oxopyrido[2,3-d]pyrimidin-8-yl)cyclohexyl]propanediamide

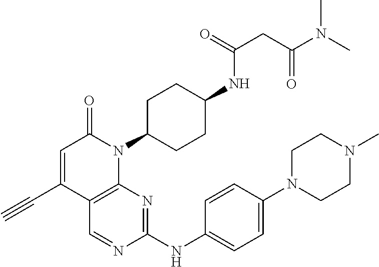 [SMILES: C#Cc1cc(=O)n([C@H]2CC[C@@H](NC(=O)CC(=O)N(C)C)CC2)c2nc(Nc3ccc(N4CCN(C)CC4)cc3)ncc12]

General procedure 15 was applied to N,N-dimethyl-N′-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanediamide (250 mg, 0.344 mmol) and potassium fluoride (200 mg, 3.44 mmol) in DMF (3.0 mL), THF (3.0 mL) and water (1.0 mL). The resulting mixture was stirred for 1 h at 60° C. The crude residue was purified by reverse phase flash column chromatography, eluting with acetonitrile (10-50%) in water (0.4% formic acid) to afford the title compound as an orange solid (67.0 mg, 34%).

(ES, m/z): [M+H]⁺=571

¹H-NMR (300 MHz, DMSO-d₆) δ 10.07 (s, 1H), 8.74 (s, 1H), 8.16 (d, J=10.6 Hz, 1H), 7.57 (d, J=8.5 Hz, 2H), 7.02-6.89 (m, 2H), 6.45 (s, 1H), 5.31 (s, 1H), 5.02 (s, 1H), 3.95 (s, 1H), 3.11 (t, J=5.0 Hz, 4H), 3.01 (s, 3H), 2.84 (d, J=15.7 Hz, 5H), 2.48 (d, J=4.7 Hz, 3H), 2.24 (s, 3H), 1.92 (d, J=13.3 Hz, 2H), 1.57 (t, J=13.4 Hz, 2H), 1.44 (d, J=12.2 Hz, 2H).

### Example 96

### 3-(Dimethylamino)-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanamide

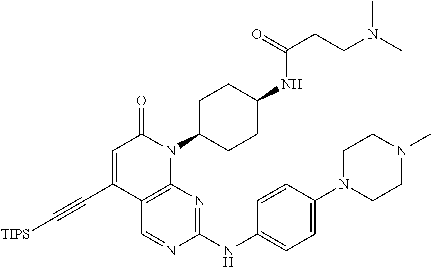

A solution of 2-{[4-(4-methylpiperazin-1-yl)phenyl]amino}-8-[(1s,4s)-4-aminocyclohexyl]-5-[2-(triisopropylsilyl)ethynyl]pyrido[2,3-d]pyrimidin-7-one (500 mg, 0.814 mmol, 1.0 eq.), 3-(dimethylamino)propanoic acid (105 mg, 0.895 mmol, 1.1 eq.), HOBT (121 mg, 0.895 mmol, 1.1 eq.), EDCl (312 mg, 1.63 mmol, 2.0 eq.) and triethylamine (247 mg, 2.44 mmol, 3.0 eq.) in dichloromethane (5.0 mL) was stirred for 2 h at 30° C. The reaction mixture was purified by reverse phase flash column chromatography eluting with acetonitrile (20-100%) in water (0.1% formic acid). The desired fractions were collected and concentrated under reduced pressure to remove most of the acetonitrile. The mixture was basified to pH 8 with a saturated aqueous sodium hydrogen carbonate solution and extracted with ethyl acetate (3×200 mL). The organic layers were washed with brine (2×200 mL), dried (Na₂SO₄) and concentrated under reduced pressure to yield the title compound as an orange solid (250 mg, 43%).

(ES, m/z): [M+H]⁺=713.

### 3-(Dimethylamino)-N-[(1s,4s)-4-(5-ethynyl-2-{[4-(4-methylpiperazin-1-yl)phenyl]amino}-7-oxopyrido[2,3-d]pyrimidin-8-yl) cyclohexyl]propanamide

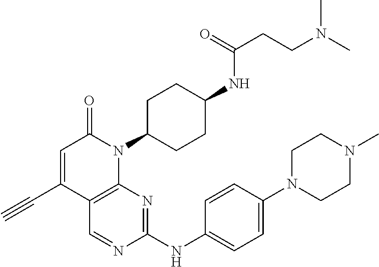 [SMILES: C#Cc1cc(=O)n([C@H]2CC[C@@H](NC(=O)CCN(C)C)CC2)c2nc(Nc3ccc(N4CCN(C)CC4)cc3)ncc12]

General procedure 15 was applied to 3-(dimethylamino)-N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]propanamide (300 mg, 0.42 mmol) with potassium fluoride (244 mg, 4.21 mmol) in THF (3.0 mL), DMF (3.0 mL) and water (0.50 mL). The resulting solution was stirred for 2 h at 70° C. and the reaction mixture was purified by reverse phase flash column chromatography, eluting with acetonitrile (20-100%) in water (0.1% formic acid). The desired fractions were collected and concentrated under reduced pressure to remove most of the acetonitrile then lyophilized to yield the title compound as an orange solid (94.7 mg, 40%).

(ES, m/z): [M+H]⁺=557.

¹H-NMR (300 MHz, DMSO-d₆) δ 10.06 (s, 1H), 8.74 (s, 1H), 8.45-8.35 (m, 1H), 7.57 (d, J=8.6 Hz, 2H), 6.94 (d, J=8.7 Hz, 2H), 6.44 (s, 1H), 5.32 (s, 1H), 5.01 (s, 1H), 3.89-3.80 (s, 2H), 3.10 (t, J=5.0 Hz, 4H), 2.75-2.69 (m, 2H), 2.57-2.53 (m, 2H), 2.51 (s, 3H), 2.38-2.34 (m, 2H), 2.29 (s, 6H), 2.24 (s, 3H), 1.95-1.85 (m, 2H), 1.58-1.53 (m, 2H), 1.41 (d, J=12.0 Hz, 2H).

### Example 97

### 4-(Dimethylamino)-N-[(1s,4s)-4-(2-{[4-(4-methylpiperazin-1-yl)phenyl]amino}-7-oxo-5-[2-(triisopropylsilyl) ethynyl]pyrido[2,3-d]pyrimidin-8-yl)cyclohexyl]butanamide

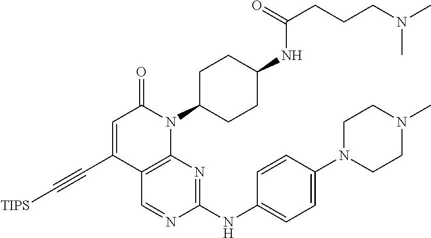

A solution of 2-{[4-(4-methylpiperazin-1-yl)phenyl]amino}-8-[(1s,4s)-4-aminocyclohexyl]-5-[2-(triisopropylsilyl)ethynyl]pyrido[2,3-d]pyrimidin-7-one (350 mg, 0.570 mmol, 1.0 eq.), 4-(dimethylamino)butanoic acid (82.3 mg, 0.627 mmol, 1.1 eq.), HOBT (84.7 mg, 0.627 mmol, 1.1 eq.), EDCl (218 mg, 1.14 mmol, 2.0 eq.) and triethylamine (173 mg, 1.71 mmol, 3.0 eq.) in dichloromethane (4.0 mL) was stirred for 2 h at 30° C. The reaction mixture was purified by reverse phase flash column chromatography, eluting with acetonitrile (20-100%) in water (0.1% formic acid). The desired fractions were collected and concentrated under reduced pressure to remove most of the acetonitrile. The mixture was basified to pH 8 with a saturated aqueous sodium hydrogen carbonate solution and extracted with ethyl acetate (3×200 mL). The organic layers were washed with brine (2×200 mL), dried (MgSO₄) and concentrated under reduced pressure to yield the title compound as a yellow solid (160 mg, 39%).

(ES, m/z): [M+H]⁺=727.

### 4-(Dimethylamino)-N-[(1s,4s)-4-(5-ethynyl-2-{[4-(4-methylpiperazin-1-yl)phenyl]amino}-7-oxopyrido[2,3-d]pyrimidin-8-yl)cyclohexyl]butanamide

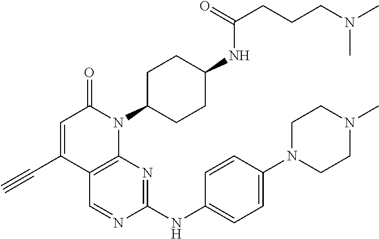 [SMILES: C#Cc1cc(=O)n([C@H]2CC[C@@H](NC(=O)CCCN(C)C)CC2)c2nc(Nc3ccc(N4CCN(C)CC4)cc3)ncc12]

General procedure 15 was applied to 4-(dimethylamino)-N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]butanamide (180 mg, 0.248 mmol) and potassium fluoride (144 mg, 2.48 mmol) in THF (2.0 mL), DMF (2.0 mL) and water (0.50 mL). The resulting solution was stirred for 2 h at 70° C. and purified by reverse phase flash column chromatography, eluting with acetonitrile (20-100%) in water (0.1% formic acid). The desired fractions were collected and concentrated under reduced pressure to remove most of the acetonitrile then lyophilized to yield the title compound as an orange solid (50.0 mg, 35%).

(ES, m/z): [M+H]⁺=571

¹H-NMR (300 MHz, DMSO-d₆) δ 10.06 (s, 1H), 8.74 (s, 1H), 7.75 (s, 1H), 7.57 (d, J=8.6 Hz, 2H), 6.94 (d, J=8.7 Hz, 2H), 6.44 (s, 1H), 5.32 (s, 1H), 5.01 (s, 1H), 3.89-3.80 (s, 2H), 3.10 (t, J=5.0 Hz, 5H), 2.89-2.75 (m, 2H), 2.55 (s, 2H), 2.38-2.30 (m, 2H), 2.24-2.15 (m, 10H), 1.99-1.88 (m, 2H), 1.69 (t, J=10.0 Hz, 2H), 1.58-1.53 (m, 2H), 1.41 (d, J=12.0 Hz, 2H).

### Example 98

### N-Methyl-N′-[(1s,4s)-4-[2-(methylsulfanyl)-7-oxo-5-[2-(triisopropylsilyl)ethynyl]pyrido[2,3-d]pyrimidin-8-yl]cyclohexyl]succinamide

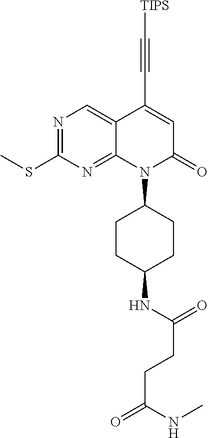

A solution of 2-(methylsulfanyl)-8-[(1s,4s)-4-aminocyclohexyl]-5-[2-(triisopropylsilyl)ethynyl]pyrido[2,3-d]pyrimidin-7-one (1.00 g, 2.12 mmol, 1.0 eq.), 3-(methylcarbamoyl)propanoic acid (360 mg, 2.76 mmol, 1.3 eq.) and N-tetramethylformamidinium hexafluorophosphate (720 mg, 2.55 mmol, 1.2 eq.) and N-methylimidazole (780 mg, 9.56 mmol, 4.5 eq.) in methanol (10 mL) was stirred for 2 h at room temperature under a nitrogen atmosphere. The reaction was quenched with water and extracted with ethyl acetate (3×10 mL). The combined organic layers were washed with brine (2×15 mL), dried (Na₂SO₄) and concentrated under reduced pressure to afford the title compound as a yellow oil (950 mg, 73%).

(ES, m/z): [M+H]⁺=584

### N-Methyl-N′-[(1s,4s)-4-{2-methanesulfonyl-7-oxo-5-[2-(triisopropylsilyl)ethynyl]pyrido[2,3-d]pyrimidin-8-yl}cyclohexyl]succinimide

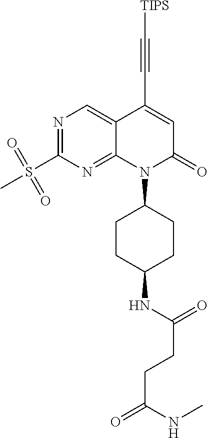

General procedure 10 was applied to N-methyl-N′-[(1s,4s)-4-[2-(methylsulfanyl)-7-oxo-5-[2-(triisopropylsilyl) ethynyl]pyrido[2,3-d]pyrimidin-8-yl]cyclohexyl]succinamide (950 mg, 1.63 mmol) and m-CPBA (618 mg, 3.58 mmol) in dichloromethane (10 mL) to afford the title compound as a yellow oil (700 mg, 63%).

(ES, m/z): [M+H]⁺=616.

### N-Methyl-N′-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]succinamide

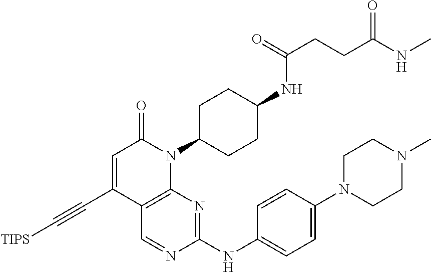

General procedure 13 was applied to N-methyl-N′-[(1s,4s)-4-{2-methanesulfonyl-7-oxo-5-[2-(triisopropylsilyl) ethynyl]pyrido[2,3-d]pyrimidin-8-yl}cyclohexyl]succinamide (700 mg, 1.14 mmol) with trifluoroacetic acid (259 mg, 2.27 mmol) and 4-(4-methylpiperazin-1-yl)aniline (326 mg, 1.71 mmol) in 2-butanol (10 mL). The resulting mixture was stirred for 2 h at 100° C. and the resulting mixture was purified by reverse phase flash column chromatography, eluting with acetonitrile (40-65%) in water (0.4% formic acid) to afford the title compound as a yellow solid (400 mg, 46%).

(ES, m/z): [M+H]⁺=727

### N-Methyl-N′-[(1s,4s)-4-(5-ethynyl-2-{[4-(4-methylpiperazin-1-yl)phenyl]amino}-7-oxopyrido[2,3-d]pyrimidin-8-yl)cyclohexyl]succinamide

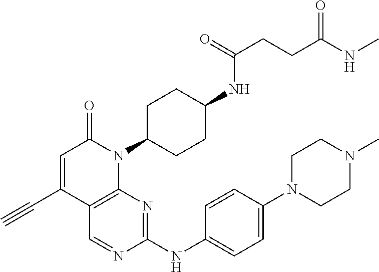 [SMILES: C#Cc1cc(=O)n([C@H]2CC[C@@H](NC(=O)CCC(=O)NC)CC2)c2nc(Nc3ccc(N4CCN(C)CC4)cc3)ncc12]

General procedure 15 was applied to N-methyl-N′-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]succinamide (250 mg, 0.344 mmol) and potassium fluoride (200 mg, 3.44 mmol) in DMF (3.0 mL) and THF (3.0 mL) water (1.0 mL) was added. The resulting mixture was stirred for 1 h at 60° C. and the residue was purified by reverse phase flash column chromatography, eluting with acetonitrile (10-50%) in water (0.4% formic acid) to afford the title compound as an orange solid (66 mg, 32%).

(ES, m/z): [M+H]⁺=571

¹H-NMR (300 MHz, DMSO-d₆) δ 10.07 (s, 1H), 8.75 (s, 1H), 7.78 (d, J=5.8 Hz, 2H), 7.57 (d, J=8.6 Hz, 2H), 6.94 (d, J=9.0 Hz, 2H), 6.45 (s, 1H), 5.31 (s, 1H), 5.02 (s, 1H), 3.89 (s, 1H), 3.10 (t, J=5.0 Hz, 4H), 2.74 (s, 2H), 2.56 (d, J=4.6 Hz, 3H), 2.46 (d, J=5.5 Hz, 6H), 2.32 (t, J=7.6 Hz, 2H), 2.23 (s, 3H), 1.91 (d, J=13.2 Hz, 2H), 1.54 (s, 2H), 1.41 (d, J=12.1 Hz, 2H).

### Example 99

### Methyl 2-{[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]carbamoyl}acetate

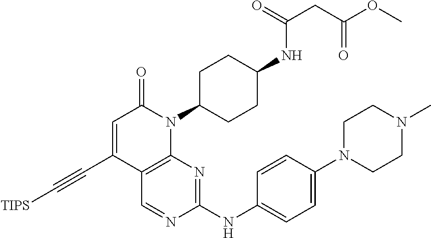

A solution of 2-{[4-(4-methylpiperazin-1-yl)phenyl]amino}-8-[(1s,4s)-4-aminocyclohexyl]-5-[2-(triisopropylsilyl)ethynyl]pyrido[2,3-d]pyrimidin-7-one (350 mg, 0.570 mmol, 1.0 eq.), 3-methoxy-3-oxopropanoic acid (74.1 mg, 0.627 mmol, 1.1 eq.), HOBT (84.7 mg, 0.627 mmol, 1.1 eq.), EDCl (218 mg, 1.14 mmol, 2.0 eq.) and triethylamine (173 mg, 1.71 mmol, 3.0 eq.) in dichloromethane (4.0 mL) was stirred for 2 h at 30° C. The reaction mixture was purified by reverse phase flash column chromatography, eluting with acetonitrile (20-100%) in water (0.1% formic acid). The desired fractions were collected and concentrated under reduced pressure to remove most of the acetonitrile. The mixture was basified to pH 8 with a saturated aqueous sodium hydrogen carbonate solution and extracted with ethyl acetate (3×200 mL). The organic layers were washed with brine (2×200 mL), dried (MgSO₄) and concentrated under reduced pressure to yield the title compound as a yellow solid (160 mg, 39%).

(ES, m/z): [M+H]⁺=714.

### 2-{[(1s,4s)-4-(5-Ethynyl-2-{[4-(4-methylpiperazin-1-yl)phenyl]amino}-7-oxopyrido[2,3-d]pyrimidin-8-yl)cyclohexyl]carbamoyl}acetic acid

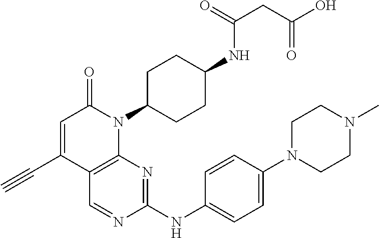 [SMILES: C#Cc1cc(=O)n([C@H]2CC[C@@H](NC(=O)CC(=O)O)CC2)c2nc(Nc3ccc(N4CCN(C)CC4)cc3)ncc12]

A solution of methyl 2-{[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]carbamoyl}acetate (160 mg, 0.224 mmol, 1.0 eq.) and lithium hydroxide solution (18.8 mg, 0.448 mmol, 2.0 eq.) in THF (2.0 mL) and water (0.50 mL) was stirred for 4 h at 30° C. The reaction mixture was purified by reverse phase column chromatography, eluting with acetonitrile (20-100%) in water (0.1% formic acid). The desired fractions were collected and concentrated under reduced pressure to remove most of the acetonitrile then lyophilized to yield the title compound as an orange solid (64.6 mg, 53%).

(ES, m/z): [M+H]⁺=544

¹H-NMR (300 MHz, DMSO-d₆) δ 10.30 (s, 1H), 8.74 (s, 1H), 8.15-7.98 (m, 2H), 7.59 (s, 1H), 6.91 (s, 2H), 6.45 (s, 1H), 5.30 (s, 1H), 5.00 (s, 1H), 4.00 (s, 2H), 3.38-3.25 (m, 1H), 3.15 (s, 4H), 2.97-2.85 (m, 2H), 2.62 (d, J=15.3 Hz, 4H), 2.33 (s, 3H), 1.90-1.70 (m, 2H), 1.60 (d, J=13.7 Hz, 2H), 1.42 (d, J=12.2 Hz, 2H).

### Example 100

### 2-(Dimethylamino)-N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]acetamide

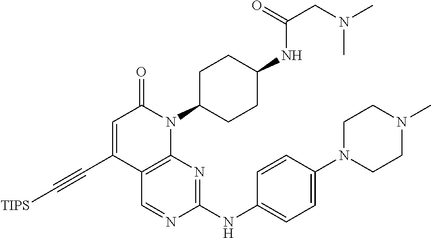

A solution of 2-{[4-(4-methylpiperazin-1-yl)phenyl]amino}-8-[(1s,4s)-4-aminocyclohexyl]-5-[2-(triisopropylsilyl)ethynyl]pyrido[2,3-d]pyrimidin-7-one (300 mg, 0.489 mmol, 1.0 eq.), dimethylglycine (55.4 mg, 0.538 mmol, 1.1 eq.), HOBT (72.6 mg, 0.538 mmol, 1.1 eq.), EDCl (187 mg, 0.978 mmol, 2.0 eq.) and triethylamine (148 mg, 1.47 mmol, 3.0 eq.) in dichloromethane (3.0 mL) was stirred for 2 h at 30° C. The reaction mixture was purified by reverse phase flash column chromatography, eluting with acetonitrile (20-100%) in water (0.1% formic acid). The fractions collected were concentrated under reduced pressure to remove most of the acetonitrile and basified to pH 8 with a saturated aqueous sodium hydrogen carbonate solution and extracted with ethyl acetate (3×200 mL). The organic layers were washed with brine (2×200 mL), dried (MgSO₄) and concentrated under reduced pressure to yield the title compound as a yellow solid (210 mg, 61%).

(ES, m/z): [M+H]⁺=699.

### 2-(Dimethylamino)-N-[(1s,4s)-4-(5-ethynyl-2-{[4-(4-methylpiperazin-1-yl)phenyl]amino}-7-oxopyrido[2,3-d]pyrimidin-8-yl)cyclohexyl]acetamide

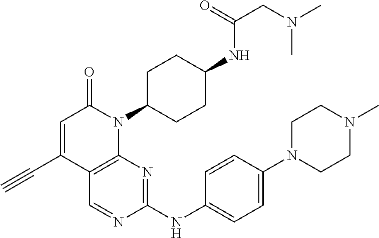 [SMILES: C#Cc1cc(=O)n([C@H]2CC[C@@H](NC(=O)CN(C)C)CC2)c2nc(Nc3ccc(N4CCN(C)CC4)cc3)ncc12]

General procedure 15 was applied to 2-(dimethylamino)-N-[(1s,4s)-4-(2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]acetamide (180 mg, 0.257 mmol) and potassium fluoride (149 mg, 2.57 mmol) in THF (2.0 mL), DMF (2.0 mL) and water (0.50 mL). The resulting solution was stirred for 2 h at 70° C. and purified by reverse phase flash column chromatography, eluting with acetonitrile (20-100%) in water (0.1% trifluoroacetic acid). The desired fractions collected were concentrated under reduced pressure to remove most of the acetonitrile then lyophilized to yield the title compound as an orange solid (43.3 mg, 31%).

(ES, m/z): [M+H]⁺=543.

¹H-NMR (300 MHz, DMSO-d₆) δ 8.77 (s, 1H), 8.56 (d, J=6.0 Hz, 1H), 7.64 (d, J=8.8 Hz, 2H), 7.03 (d, J=8.9 Hz, 2H), 6.46 (s, 1H), 5.35 (s, 1H), 4.99 (s, 1H), 4.01 (d, J=13.0 Hz, 3H), 3.80 (d, J=13.0 Hz, 2H), 3.17 (d, J=12.4 Hz, 2H), 2.85 (d, J=12.4 Hz, 11H), 1.96 (d, J=13.2 Hz, 2H), 1.64 (d, J=14.1 Hz, 2H), 1.48 (d, J=12.0 Hz, 3H).

### Example 101

### tert-Butyl N-Methyl-N-[(1s,4s)-4-[2-(methylsulfanyl)-7-oxo-5-[2-(triisopropylsilyl)ethynyl]pyrido[2,3-d]pyrimidin-8-yl]cyclohexyl]carbamate

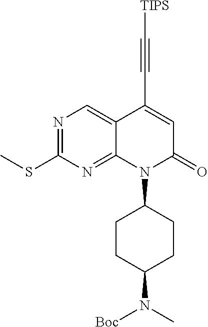

General procedure 18 was applied to 2-(methylsulfanyl)-5-[2-(triisopropylsilyl)ethynyl]-8H-pyrido[2,3-d]pyrimidin-7-one (2.50 g, 6.69 mmol), triphenylphosphine (5.26 g, 20.1 mmol), DIAD (4.06 g, 20.1 mmol) and tert-butyl N-methyl-N-[(1r,4r)-4-hydroxycyclohexyl]carbamate (2.30 g, 10.0 mmol) in THF (25 mL). The residue was purified by flash column chromatography, eluting with ethyl acetate (2%) in 40-60 petroleum ether to afford the title compound as a yellow solid (2.00 g, 51%).

(ES, m/z): [M+H]⁺=585.

### tert-Butyl N-methyl-N-[(1s,4s)-4-{2-methanesulfonyl-7-oxo-5-[2-(triisopropylsilyl)ethynyl]pyrido[2,3-d]pyrimidin-8-yl}cyclohexyl]carbamate

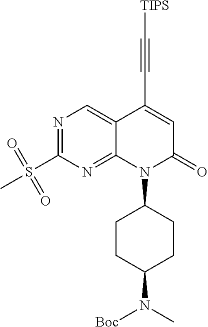

General procedure 10 was applied to tert-butyl N-methyl-N-[(1s,4s)-4-[2-(methylsulfanyl)-7-oxo-5-[2-(triisopropylsilyl)ethynyl]pyrido[2,3-d]pyrimidin-8-yl]cyclohexyl]carbamate (1.00 g, 1.71 mmol) and m-CPBA (649 mg, 3.76 mmol) in dichloromethane (10 mL) to yield the title compound as a yellow solid (1.00 g, 95%) which was used in the next step without further purification.

(ES, m/z): [M+H]⁺=617

### 2-[(2-Methoxyphenyl)amino]-8-[(1s,4s)-4-(methylamino)cyclohexyl]-5-[2-(triisopropylsilyl)ethynyl]pyrido[2,3-d]pyrimidin-7-one

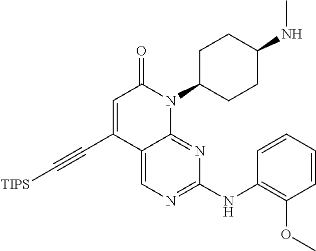

General procedure 13 was applied to tert-butyl N-methyl-N-[(1s,4s)-4-{2-methanesulfonyl-7-oxo-5-[2-(triisopropylsilyl)ethynyl]pyrido[2,3-d]pyridin-8-yl}cyclohexyl]carbamate (1.00 g, 1.62 mmol) with trifluoroacetic acid (2.22 g, 19.5 mmol) and o-anisidine (0.30 g, 2.431 mmol) in 2-butanol (10 mL). The resulting mixture was stirred at 100° C. for 2 h and purified by reverse phase flash column chromatography, eluting with acetonitrile (50-60%) in water (0.1% formic acid) to afford the title compound as a brown solid (150 mg, 16%).

(ES, m/z): [M+H]⁺=560

### 2-(Dimethylamino)-N-methyl-N-[(1s,4s)-4-{2-[(2-methoxyphenyl)amino]-7-oxo-5-[2-(triisopropylsilyl)ethynyl]pyrido[2,3-d]pyrimidin-8-yl}cyclohexyl]acetamide

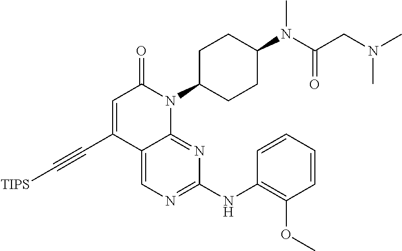

To a stirred solution 2-[(2-methoxyphenyl)amino]-8-[(1s,4s)-4-(methylamino)cyclohexyl]-5-[2-(triisopropylsilyl)ethynyl]pyrido[2,3-d]pyrimidin-7-one (150 mg, 0.268 mmol, 1.0 eq.) and dimethylglycine (30.4 mg, 0.295 mmol, 1.1 eq.) in DMF (2.0 mL) was added HOBT (39.8 mg, 0.295 mmol, 1.1 eq.), EDCl (103 mg, 0.536 mmol, 2.0 eq.) and triethylamine (81.3 mg, 0.804 mmol, 3.0 eq.). The resulting mixture was stirred for 2 h at room temperature, diluted with water (8 mL) and extracted with ethyl acetate (3×10 mL). The combined organic layers were washed with brine (3×15 mL), dried (Na₂SO₄) and concentrated under reduced pressure to afford the title compound as a yellow oil (180 mg, 100%).

(ES, m/z): [M+H]⁺=645

### 2-(Dimethylamino)-N-methyl-N-[(1s,4s)-4-{5-ethynyl-2-[(2-methoxyphenyl)amino]-7-oxopyrido[2,3-d]pyrimidin-8-yl}cyclohexyl]acetamide

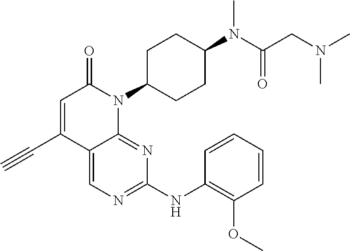 [SMILES: C#Cc1cc(=O)n([C@H]2CC[C@@H](N(C)C(=O)CN(C)C)CC2)c2nc(Nc3ccccc3OC)ncc12]

General procedure 15 was applied to 2-(dimethylamino)-N-methyl-N-[(1s,4s)-4-{2-[(2-methoxyphenyl)amino]-7-oxo-5-[2-(triisopropylsilyl)ethynyl]pyrido[2,3-d]pyrimidin-8-yl}cyclohexyl]acetamide (150 mg, 0.233 mmol) and potassium fluoride (135 mg, 2.33 mmol) in DMF (2.0 mL), THF (2.0 mL) and water (0.50 mL). The resulting mixture was stirred at 60° C. for 1 h and purified by reverse phase flash column chromatography, eluting with acetonitrile (30-40%) in water (0.05% trifluoroacetic acid) to afford the title compound as a yellow-green solid (32.0 mg, 28%).

(ES, m/z): [M+H]⁺=489

¹H-NMR (300 MHz, CDCl₃) δ 9.29 (d, J=2.9 Hz, 1H), 8.68 (s, 1H), 8.18 (s, 1H), 7.11-7.01 (m, 2H), 6.95 (s, 1H), 6.89 (s, 1H), 5.73 (s, 1H), 4.58 (s, 1H), 3.93 (s, 3H), 3.65 (d, J=2.3 Hz, 1H), 3.47 (d, J=9.3 Hz, 2H), 2.94 (d, J=11.7 Hz, 3H), 2.62 (d, J=7.4 Hz, 6H), 2.28 (d, J=11.1 Hz, 2H), 2.02 (s, 2H), 1.89 (d, J=13.6 Hz, 2H), 1.78 (d, J=14.6 Hz, 2H).

### Example 102

### N-[(1r,4r)-4-[6-Bromo-2-(methylsulfanyl)-7-oxo-5-[2-(triisopropylsilyl)ethynyl]pyrido[2,3-d]pyrimidin-8-yl]cyclohexyl]acetamide

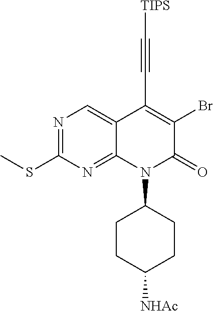

A mixture of N-[(1r,4r)-4-[2-(methylsulfanyl)-7-oxo-5-[2-(triisopropylsilyl)ethynyl]pyrido[2,3-d]pyrimidin-8-yl]cyclohexyl]acetamide (1.00 g, 1.95 mmol, 1.0 eq.), N-bromosuccinimide (521 mg, 2.93 mmol, 1.5 eq.) and acetic acid (23.4 mg, 0.390 mmol, 0.2 eq.) in acetonitrile (10 mL) was stirred at 85° C. for 40 h. The reaction mixture was cooled to room temperature, diluted with water (10 mL) and extracted with dichloromethane (3×20 mL). The combined organic layers were washed with brine (3×20 mL), dried (Na₂SO₄) and concentrated under reduced pressure to afford the title compound as a yellow solid (950 mg, 66%).

(ES, m/z): [M+H]⁺=591

### N-[(1r,4r)-4-[6-Methyl-2-(methylsulfanyl)-7-oxo-5-[2-(triisopropylsilyl)ethynyl]pyrido[2,3-d]pyrimidin-8-yl]cyclohexyl]acetamide

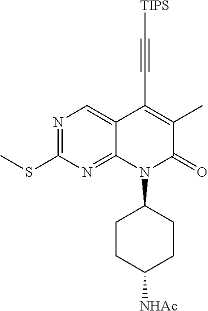

General procedure 17 was applied to N-[(1r,4r)-4-[6-bromo-2-(methylsulfanyl)-7-oxo-5-[2-(triisopropylsilyl) ethynyl]pyrido[2,3-d]pyrimidin-8-yl]cyclohexyl]acetamide (850 mg, 1.15 mmol), potassium carbonate (318 mg, 2.30 mmol) and (1,1′-Bis(diphenylphosphino)ferrocene)palladium(II) dichloride (84.1 mg, 0.115 mmol) and methylboronic acid (137 mg, 2.30 mmol) in dioxane (10 mL) and water (2.0 mL). The crude residue was purified by reverse phase flash column chromatography, eluting with acetonitrile (95-100%) in water (0.1% formic acid) to afford the title compound as a yellow solid (300 mg, 49%).

(ES, m/z): [M+H]⁺=527.

### N-[(1r,4r)-4-{2-Methanesulfonyl-6-methyl-7-oxo-5-[2-(triisopropylsilyl)ethynyl]pyrido[2,3-d]pyrimidin-8-yl}cyclohexyl]acetamide

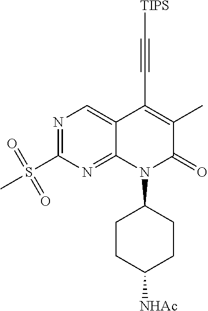

General procedure 10 was applied to N-[(1r,4r)-4-[6-methyl-2-(methylsulfanyl)-7-oxo-5-[2-(triisopropylsilyl)ethynyl]pyrido[2,3-d]pyrimidin-8-yl]cyclohexyl]acetamide (300 mg, 0.553 mmol) with m-CPBA (210 mg, 1.28 mmol) in dichloromethane (3.0 mL) to yield the title compound as a yellow solid (350 mg, >100%) which was used in the next step directly without further purification.

(ES, m/z): [M+H]⁺=558

### N-[(1r,4r)-4-(6-Methyl-2-{[4-(4-methylpiperazin-1-yl)phenyl]amino}-7-oxo-5-[2-(triisopropylsilyl)ethynyl]pyrido[2,3-d]pyrimidin-8-yl) cyclohexyl]acetamide

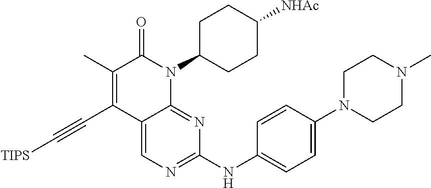

General procedure 13 was applied to N-[(1r,4r)-4-{2-methanesulfonyl-6-methyl-7-oxo-5-[2-(triisopropylsilyl)ethynyl]pyrido[2,3-d]pyrimidin-8-yl}cyclohexyl]acetamide (350 mg, 0.626 mmol) with trifluoroacetic acid (143 mg, 1.25 mmol) and 4-(4-methylpiperazin-1-yl)aniline (180 mg, 0.939 mmol) in 2-butanol (4.0 mL). The resulting mixture was stirred at 100° C. for 16 h. The reaction mixture was purified by reverse phase flash column chromatography, eluting with acetonitrile (50-70%) in water (0.1% formic acid) to yield the title compound as a brown solid (130 mg, 31%).

(ES, m/z): [M+H]⁺=670

### N-[(1r,4r)-4-(5-Ethynyl-6-methyl-2-{[4-(4-methylpiperazin-1-yl)phenyl]amino}-7-oxopyrido[2,3-d]pyrimidin-8-yl)cyclohexyl]acetamide

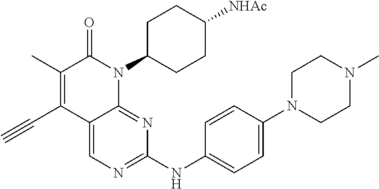 [SMILES: C#Cc1c(C)c(=O)n([C@H]2CC[C@H](NC(C)=O)CC2)c2nc(Nc3ccc(N4CCN(C)CC4)cc3)ncc12]

General procedure 15 was applied to N-[(1r,4r)-4-(6-methyl-2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]acetamide (130 mg, 0.194 mmol) and potassium fluoride (113 mg, 1.94 mmol) in THF (1.0 mL), DMF (1.0 mL) and water (0.2.0 mL). The resulting mixture was stirred at 60° C. for 1 h. The reaction mixture was purified by reverse phase flash column chromatography, eluting with acetonitrile in water (0.1% formic acid) to afford the title compound as a brown yellow solid (30.0 mg, 30%).

(ES, m/z): [M+H]⁺=514.

¹H-NMR (300 MHz, DMSO-d₆) δ 9.94 (s, 1H), 9.63 (s, 1H), 8.76 (s, 1H), 7.86 (d, J=7.6 Hz, 1H), 7.66 (d, J=8.6 Hz, 2H), 7.10 (s, 2H), 5.22 (s, 2H), 3.87 (d, J=12.0 Hz, 4H), 3.53 (d, J=11.9 Hz, 2H), 3.27-3.09 (m, 2H), 2.97 (d, J=12.6 Hz, 2H), 2.88 (d, J=4.3 Hz, 3H), 2.21 (s, 3H), 1.92 (s, 2H), 1.84 (s, 3H), 1.60 (s, 2H), 1.43-1.23 (m, 2H).

### Example 103

### N-[(1s,4s)-4-[6-Bromo-2-(methylsulfanyl)-7-oxo-5-[2-(triisopropylsilyl)ethynyl]pyrido[2,3-d]pyrimidin-8-yl]cyclohexyl]acetamide

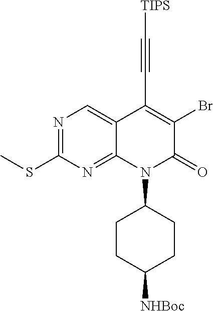

A solution of N-[(1s,4s)-4-[2-(methylsulfanyl)-7-oxo-5-[2-(triisopropylsilyl)ethynyl]pyrido[2,3-d]pyrimidin-8-yl]cyclohexyl]acetamide (1.30 g, 2.54 mmol, 1.0 eq.), N-bromosuccinimide (677 mg, 3.80 mmol, 1.5 eq.) and acetic acid (30.5 mg, 0.507 mmol, 0.2 eq.) in acetonitrile (15 mL) was stirred at 85° C. for 40 h. The reaction mixture was allowed to cool down to room temperature and purified by flash column chromatography, eluting with ethyl acetate (50%) in 40-60 petroleum ether to afford the title compound as a yellow solid (850 mg, 48%).

(ES, m/z): [M+H]=591

### N-[(1s,4s)-4-[6-Methyl-2-(methylsulfanyl)-7-oxo-5-[2-(triisopropylsilyl)ethynyl]pyrido[2,3-d]pyrimidin-8-yl]cyclohexyl]acetamide

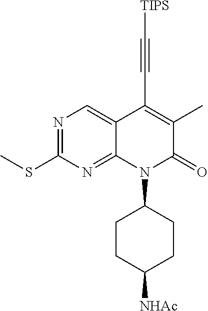

General procedure 17 was applied to N-[(1s,4s)-4-[6-bromo-2-(methylsulfanyl)-7-oxo-5-[2-(triisopropylsilyl)ethynyl]pyrido[2,3-d]pyrimidin-8-yl]cyclohexyl]acetamide (850 mg, 1.22 mmol), potassium carbonate (337 mg, 2.44 mmol) and (1,1′-bis(diphenylphosphino)ferrocene)palladium(II) dichloride (89.3 mg, 0.122 mmol) and methylboronic acid (146 mg, 2.44 mmol) in dioxane (10 mL) and water (2.0 mL). The crude residue was purified by reverse phase flash column chromatography, eluting with acetonitrile (95-100%) in water (0.1% formic acid) afford the title compound as a yellow green solid (340 mg, 53%).

(ES, m/z): [M+H]⁺=527.

### N-[(1s,4s)-4-{2-methanesulfonyl-6-methyl-7-oxo-5-[2-(triisopropylsilyl)ethynyl]pyrido[2,3-d]pyrimidin-8-yl}cyclohexyl]acetamide

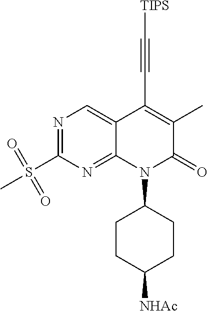

General procedure 10 was applied to N-[(1s,4s)-4-[6-methyl-2-(methylsulfanyl)-7-oxo-5-[2-(triisopropylsilyl)ethynyl]pyrido[2,3-d]pyrimidin-8-yl]cyclohexyl]acetamide (340 mg, 0.645 mmol) and m-CPBA (245 mg, 1.42 mmol) in dichloromethane (5 mL) to afford the title compound as a light yellow solid (360 mg, 100%).

(ES, m/z): [M+H]⁺=559

### N-[(1s,4s)-4-(6-methyl-2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]acetamide

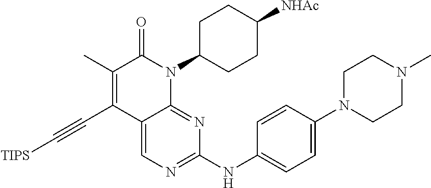

General procedure 13 was applied to N-[(1s,4s)-4-{2-methanesulfonyl-6-methyl-7-oxo-5-[2-(triisopropylsilyl) ethynyl]pyrido[2,3-d]pyrimidin-8-yl}cyclohexyl]acetamide (350 mg, 0.626 mmol) with trifluoroacetic acid (143 mg, 1.25 mmol) and 4-(4-methylpiperazin-1-yl)aniline (180 mg, 0.939 mmol) in 2-butanol (4 mL). The resulting mixture was stirred for 16 h at 100° C. The mixture was purified by reverse phase flash column chromatography eluting with acetonitrile (50-70%) in water (0.1% formic acid) to yield the title compound as a yellow solid (180 mg, 43%).

(ES, m/z): [M+H]⁺=670

### N-[(1s,4s)-4-(5-ethynyl-6-methyl-2-{[4-(4-methylpiperazin-1-yl)phenyl]amino}-7-oxopyrido[2,3-d]pyrimidin-8-yl)cyclohexyl]acetamide

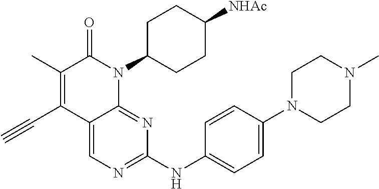 [SMILES: C#Cc1c(C)c(=O)n([C@H]2CC[C@@H](NC(C)=O)CC2)c2nc(Nc3ccc(N4CCN(C)CC4)cc3)ncc12]

General procedure 15 was applied to N-[(1s,4s)-4-(6-methyl-2-{[4-(4-methylpiperazin-1-yl)phenyl]amino}-7-oxo-5-[2-(triisopropylsilyl)ethynyl]pyrido[2,3-d]pyrimidin-8-yl)cyclohexyl]acetamide (180 mg, 0.269 mmol) and potassium fluoride (156 mg, 2.69 mmol) in DMF (2.0 mL), THF (2.0 mL) and water (0.50 mL). The resulting mixture was stirred for 1 h at 60° C. The mixture was purified by reverse phase flash column chromatography eluting with acetonitrile (30-40%) in water (0.1% formic acid), to afford the title compound as a yellow solid (103 mg, 75%).

(ES, m/z): [M+H]⁺=514

¹H-NMR (300 MHz, DMSO-d₆) δ 9.88 (s, 1H), 8.74 (s, 1H), 7.79 (s, 1H), 7.59 (d, J=8.7 Hz, 2H), 6.94 (d, J=8.9 Hz, 2H), 5.37 (s, 1H), 5.20 (s, 1H), 3.91 (s, 1H), 3.10 (t, J=5.0 Hz, 4H), 2.80 (s, 2H), 2.47 (d, J=5.0 Hz, 4H), 2.22 (d, J=8.3 Hz, 6H), 1.95 (s, 2H), 1.92 (s, 3H), 1.57 (d, J=14.3 Hz, 2H), 1.41 (d, J=12.0 Hz, 2H).

### Example 104

### 3-[2-(methylsulfanyl)-7-oxo-5-[2-(triisopropylsilyl)ethynyl]pyrido[2,3-d]pyrimidin-8-yl]pyrrolidin-2-one

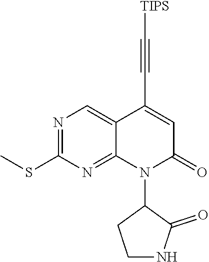

General procedure 18 was applied to 2-(methylsulfanyl)-5-[2-(triisopropylsilyl)ethynyl]-8H-pyrido[2,3-d]pyrimidin-7-one (1.00 g, 2.678 mmol), triphenylphosphine (1.40 g, 5.35 mmol), DIAD (1.08 g, 5.35 mmol) and 3-hydroxypyrrolidin-2-one (298 mg, 2.95 mmol) in THF (15 mL). The residue was purified by flash column chromatography, eluting with ethyl acetate (6%) in 40-60 petroleum ether to afford the title compound as a white solid (440 mg, 36%).

(ES, m/z): [M+H]⁺=457.2

### 3-{2-methanesulfonyl-7-oxo-5-[2-(triisopropylsilyl)ethynyl]pyrido[2,3-d]pyrimidin-8-yl}pyrrolidin-2-one

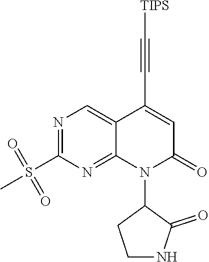

General procedure 10 was applied to 3-[2-(methylsulfanyl)-7-oxo-5-[2-(triisopropylsilyl)ethynyl]pyrido[2,3-d]pyrimidin-8-yl]pyrrolidin-2-one (440 mg, 0.963 mmol) and m-CPBA (366 mg, 2.12 mmol) in dichloromethane (10 mL) to afford the title compound as a yellow solid (470 mg, 100%) as a yellow solid, which was used without further purification.

(ES, m/z): [M+H]⁺=489.1

### 3-(2-{[4-(4-methylpiperazin-1-yl)phenyl]amino}-7-oxo-5-[2-(triisopropylsilyl)ethynyl]pyrido[2,3-d]pyrimidin-8-yl)pyrrolidin-2-one

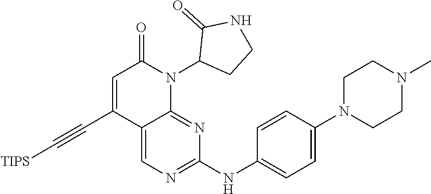

General procedure 13 was applied to 3-{2-methanesulfonyl-7-oxo-5-[2-(triisopropylsilyl)ethynyl]pyrido[2,3-d]pyrimidin-8-yl}pyrrolidin-2-one (420 mg, 0.859 mmol), 4-(4-methylpiperazin-1-yl)aniline (246 mg, 1.29 mmol) and trifluoroacetic acid (196 mg, 1.72 mmol) in 2-butanol (3.0 mL). The reaction was stirred for 6 h at 70° C. and purified by reverse phase flash column chromatography eluting with acetonitrile (30-60%) in water (0.1% formic acid) to afford the title compound as an orange solid (290 mg, 56%).

(ES, m/z): [M+H]⁺=600.3

### 3-(5-ethynyl-2-{[4-(4-methylpiperazin-1-yl)phenyl]amino}-7-oxopyrido[2,3-d]pyrimidin-8-yl)pyrrolidin-2-one

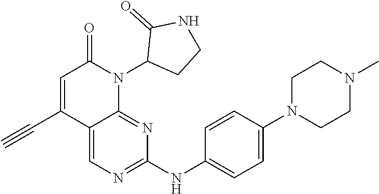 [SMILES: C#Cc1cc(=O)n(C2CCNC2=O)c2nc(Nc3ccc(N4CCN(C)CC4)cc3)ncc12]

General procedure 15 was applied to 3-(2-{[4-(4-methylpiperazin-1-yl)phenyl]amino}-7-oxo-5-[2-(triisopropylsilyl)ethynyl]pyrido[2,3-d]pyrimidin-8-yl)pyrrolidin-2-one (240 mg, 0.400 mmol) and potassium fluoride (232 mg, 4.00 mmol) in DMF (1.0 mL), THF (1.0 mL) and water (0.20 mL). The reaction was stirred for 2 h at 70° C., before being purified by reverse phase flash column chromatography eluting with acetonitrile (20-30%) in water (0.1% formic acid) to afford the title compound as an orange solid (53.5 mg, 30%).

(ES, m/z): [M+H]⁺=444.2

¹H NMR (400 MHz, DMSO-d₆) δ 9.92 (s, 1H), 9.14 (s, 1H), 8.13 (s, 1H), 7.69 (s, 2H), 6.98-6.90 (m, 3H), 5.79 (s, 1H), 5.07 (s, 1H), 3.31 (s, 2H), 3.10 (t, J=5.0 Hz, 4H), 2.66 (tt, J=8.4, 3.8 Hz, 1H), 2.46 (d, J=10.0 Hz, 4H), 2.23 (s, 3H), 2.04 (dq, J=12.9, 8.4 Hz, 1H).

### Example 105

### 2-(methylsulfanyl)-8-(oxolan-3-yl)-5-[2-(triisopropylsilyl)ethynyl]pyrido[2,3-d]pyrimidin-7-one

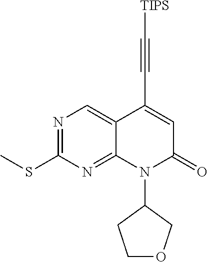

General procedure 18 was applied to 2-(methylsulfanyl)-5-[2-(triisopropylsilyl)ethynyl]-8H-pyrido[2,3-d]pyrimidin-7-one (900 mg, 2.41 mmol), 3-hydroxytetrahydrofuran (233 mg, 2.65 mmol), triphenylphosphine (1.26 g, 4.82 mmol) and DIAD (974 mg, 4.82 mmol) in THF (2.0 mL). The residue was purified by flash column chromatography, eluting with ethyl acetate (6%) in 40-60 petroleum ether to afford the title compound as a yellow oil (735 mg, 69%).

(ES, m/z): [M+H]⁺=444.2

### 2-methanesulfonyl-8-(oxolan-3-yl)-5-[2-(triisopropylsilyl)ethynyl]pyrido[2,3-d]pyrimidin-7-one

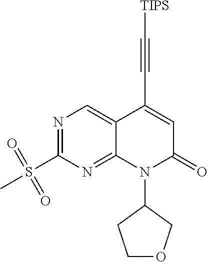

To a stirred solution of 2-(methylsulfanyl)-8-(oxolan-3-yl)-5-[2-(triisopropylsilyl)ethynyl]pyrido[2,3-d]pyrimidin-7-one (660 mg, 1.49 mmol) and m-CPBA (565 mg, 3.27 mmol) in dichloromethane (10 mL) was added to afford the title compound as a yellow solid (650 mg, 92%), which was used without further purification.

(ES, m/z): [M+H]⁺=476.1

### 2-{[4-(4-methylpiperazin-1-yl)phenyl]amino}-8-(oxolan-3-yl)-5-[2-(triisopropylsilyl)ethynyl]pyrido[2,3-d]pyrimidin-7-one

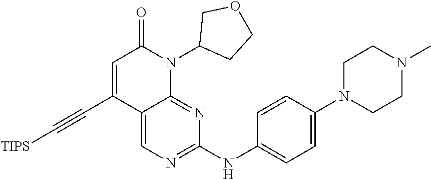

General procedure 13 was applied to 2-methanesulfonyl-8-(oxolan-3-yl)-5-[2-(triisopropylsilyl)ethynyl]pyrido[2,3-d]pyrimidin-7-one (600 mg, 1.26 mmol), 4-(4-methylpiperazin-1-yl)aniline (362 mg, 1.89 mmol) and trifluoroacetic acid (287 mg, 2.52 mmol) in 2-butanol (3.0 mL). The reaction was stirred for 6 h at 70° C. The residue was purified by reverse phase flash column chromatography eluting with acetonitrile (40-50%) in water (0.1% formic acid) to afford the title compound as an orange solid (431 mg, 58%).

(ES, m/z): [M+H]⁺=587.3

### 5-ethynyl-2-{[4-(4-methylpiperazin-1-yl)phenyl]amino}-8-(oxolan-3-yl)pyrido[2,3-d]pyrimidin-7-one

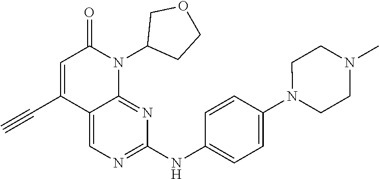 [SMILES: C#Cc1cc(=O)n(C2CCOC2)c2nc(Nc3ccc(N4CCN(C)CC4)cc3)ncc12]

General procedure 15 was applied to 2-{[4-(4-methylpiperazin-1-yl)phenyl]amino}-8-(oxolan-3-yl)-5-[2-(triisopropylsilyl)ethynyl]pyrido[2,3-d]pyrimidin-7-one (380 mg, 0.648 mmol) and potassium fluoride (376 mg, 6.48 mmol) in DMF (1.0 mL), THF (1.0 mL) and water (100 μL). The reaction was stirred for 2 h at 60° C. before being purified by reverse phase flash column chromatography eluting with acetonitrile (20-30%) in water (0.1% formic acid) to afford the title compound as an orange solid (228 mg, 76%).

(ES, m/z): [M+H]⁺=431.3

¹H NMR (400 MHz, DMSO-d₆) 59.93 (s, 1H), 9.13 (s, 1H), 7.69 (s, 2H), 6.94 (d, J=9.0 Hz, 2H), 6.90 (s, 1H), 5.74 (s, 1H), 5.05 (s, 1H), 3.95 (dd, J=10.5, 4.6 Hz, 1H), 3.86 (dd, J=13.9, 9.0 Hz, 2H), 3.81-3.76 (m, 1H), 3.10 (t, J=5.0 Hz, 4H), 2.47 (t, J=5.1 Hz, 4H), 2.30 (dd, J=14.3, 6.8 Hz, 1H), 2.23 (s, 3H), 2.10-2.02 (m, 1H).

### Example 106

### tert-butyl 3-[2-(methylsulfanyl)-7-oxo-5-[2-(triisopropylsilyl)ethynyl]pyrido[2,3-d]pyrimidin-8-yl]pyrrolidine-1-carboxylate

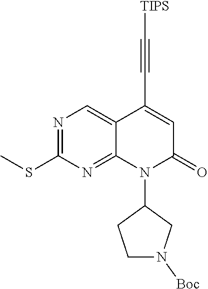

General procedure 18 was applied to 2-(methylsulfanyl)-5-[2-(triisopropylsilyl)ethynyl]-8H-pyrido[2,3-d]pyrimidin-7-one (1.00 g, 2.68 mmol), tert-butyl 3-hydroxypyrrolidine-1-carboxylate (550 mg, 2.95 mmol), triphenylphosphine (1.40 g, 5.35 mmol) and DIAD (1.06 mL, 5.35 mmol) in THF (3.0 mL). The residue was purified by flash column chromatography eluting with ethyl acetate (10%) in 40-60 petroleum ether to afford the title compound as a colourless oil (1.15 g, 79%).

(ES, m/z): [M+H]⁺=543

### tert-butyl 3-{2-methanesulfonyl-7-oxo-5-[2-(triisopropylsilyl)ethynyl]pyrido[2,3-d]pyrimidin-8-yl}pyrrolidine-1-carboxylate

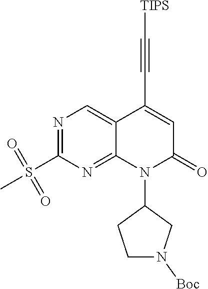

General procedure 10 was applied to tert-butyl 3-[2-(methylsulfanyl)-7-oxo-5-[2-(triisopropylsilyl)ethynyl]pyrido[2,3-d]pyrimidin-8-yl]pyrrolidine-1-carboxylate (600 mg, 1.11 mmol) and m-CPBA (420 mg, 2.43 mmol) in dichloromethane (10 mL) to yield the title compound as a colourless oil (587 mg, 92%).

(ES, m/z): [M+H]⁺=575

### 2-{[4-(4-methylpiperazin-1-yl)phenyl]amino}-8-(pyrrolidin-3-yl)-5-[2-(triisopropylsilyl)ethynyl]pyrido[2,3-d]pyrimidin-7-one

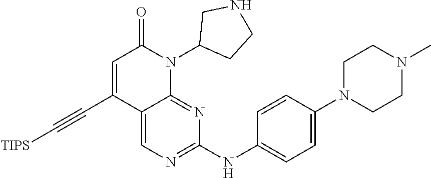

General procedure 13 was applied to tert-butyl 3-{2-methanesulfonyl-7-oxo-5-[2-(triisopropylsilyl)ethynyl]pyrido[2,3-d]pyrimidin-8-yl}pyrrolidine-1-carboxylate (1.42 g, 2.47 mmol), 4-(4-methylpiperazin-1-yl)aniline (710 mg, 3.71 mmol) and trifluoroacetic acid (0.37 mL, 4.94 mmol) in dioxane (20 mL). The reaction was stirred for 4 h at 60° C. before being purified by reverse phase flash column chromatography eluting with acetonitrile (10-80%) in water (0.1% formic acid) to yield the title compound as a yellow oil (530 mg, 37%).

(ES, m/z): [M+H]⁺=586

### 5-ethynyl-2-{[4-(4-methylpiperazin-1-yl)phenyl]amino}-8-(pyrrolidin-3-yl)pyrido[2,3-]pyrimidin-7-one

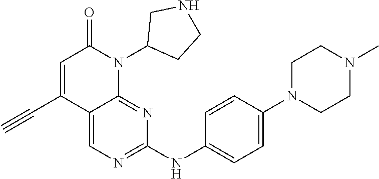 [SMILES: C#Cc1cc(=O)n(C2CCNC2)c2nc(Nc3ccc(N4CCN(C)CC4)cc3)ncc12]

General procedure 15 was applied to 2-{[4-(4-methylpiperazin-1-yl)phenyl]amino}-8-(pyrrolidin-3-yl)-5-[2-(triisopropylsilyl) ethynyl]pyrido[2,3-d]pyrimidin-7-one (142 mg, 0.242 mmol) and potassium fluoride (141 mg, 2.42 mmol) in THF (3.0 mL) and water (0.50 mL). The reaction was stirred for 4 h at 60° C. before being purified by reverse phase flash column chromatography eluting with acetonitrile (10-80%) in water (0.1% formic acid) to yield the title compound as a yellow solid (3.00 mg, 3%).

(ES, m/z): [M+H]⁺=430

¹H NMR (300 MHz, DMSO-d₆) δ 9.95 (s, 1H), 9.14 (d, J=5.2 Hz, 1H), 8.32 (s, 1H), 7.70 (s, 2H), 7.00-6.81 (m, 3H), 5.06 (s, 1H), 3.51-3.26 (m, 3H), 3.22-2.96 (m, 7H), 2.46 (t, J=5.0 Hz, 4H), 2.23 (s, 4H), 2.00 (s, 1H).

### Example 107

### 2-{[4-(4-methylpiperazin-1-yl)phenyl]amino}-8-(1-methylpyrrolidin-3-yl)-5-[2-(triisopropylsilyl)ethynyl]pyrido[2,3-d]pyrimidin-7-one

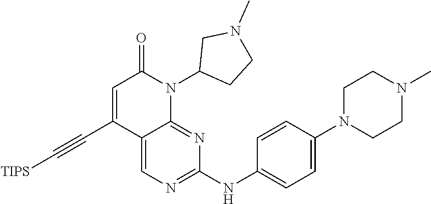

A solution of 2-{[4-(4-methylpiperazin-1-yl)phenyl]amino}-8-(pyrrolidin-3-yl)-5-[2-(triisopropylsilyl) ethynyl]pyrido[2,3-d]pyrimidin-7-one (225 mg, 0.384 mmol, 1.0 eq.), formaldehyde (115 mg, 3.84 mmol, 10 eq.) in acetic acid (0.8 mL) and methanol (4 mL) was stirred for 5 min at 0° C. To the above mixture was added sodium triacetoxyborohydride (488 mg, 2.30 mmol, 6.0 eq.) in methanol (2.0 mL) dropwise over 5 min at 0° C. The resulting mixture was stirred for 1 h at room temperature before being purified by reverse phase flash column chromatography eluting with acetonitrile (5-80%) in water (0.1% formic acid) to yield the title compound as a red solid (123 mg, 53%).

(ES, m/z): [M+H]⁺=600

### 5-ethynyl-2-{[4-(4-methylpiperazin-1-yl)phenyl]amino}-8-(1-methylpyrrolidin-3-yl)pyrido[2,3-d]pyrimidin-7-one

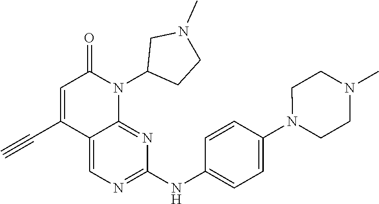 [SMILES: C#Cc1cc(=O)n(C2CCN(C)C2)c2nc(Nc3ccc(N4CCN(C)CC4)cc3)ncc12]

General procedure 15 was applied to 2-{[4-(4-methylpiperazin-1-yl)phenyl]amino}-8-(1-methylpyrrolidin-3-yl)-5-[2-(triisopropylsilyl)ethynyl]pyrido[2,3-d]pyrimidin-7-one (286 mg, 0.477 mmol) and potassium fluoride (277 mg, 4.77 mmol) in THF (3.0 mL), water (0.50 mL) and DMF (1.5 mL). The reaction was stirred for 1 h at 60° C. The residue was purified by reverse phase flash column chromatography eluting with acetonitrile (5-40%) in water (0.1% formic acid) to yield the title compound as a red solid (56.0 mg, 26%).

(ES, m/z): [M+H]⁺=444

¹H NMR (300 MHz, DMSO-d₆) δ 9.93 (s, 1H), 9.12 (s, 1H), 7.70 (d, J=8.3 Hz, 2H), 6.94 (d, J=8.7 Hz, 2H), 6.88 (s, 1H), 5.57 (s, 1H), 5.04 (s, 1H), 3.11 (t, J=4.8 Hz, 4H), 2.88-2.69 (m, 3H), 2.48 (d, J=4.4 Hz, 4H), 2.42-2.35 (m, 2H), 2.30 (s, 3H), 2.24 (s, 3H), 2.00 (q, J=6.5, 5.6 Hz, 2H), 1.91-1.80 (m, 1H), 1.61-1.39 (m, 2H), 1.24 (s, 9H), 0.90-0.73 (m, 3H).

### Example 108

### 4-[2-(methylsulfanyl)-7-oxo-5-[2-(triisopropylsilyl)ethynyl]pyrido[2,3-d]pyrimidin-8-yl]pyrrolidin-2-one

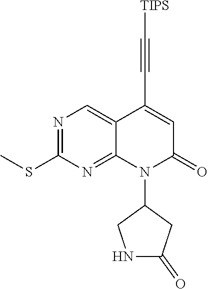

General procedure 18 was applied to 2-(methylsulfanyl)-5-[2-(triisopropylsilyl)ethynyl]-8H-pyrido[2,3-d]pyrimidin-7-one (1.00 g, 2.68 mmol), 4-hydroxypyrrolidin-2-one (300 mg, 2.95 mmol), DIAD (1.08 g, 5.35 mmol) and triphenylphosphine (1.40 g, 5.35 mmol) in dichloromethane (20 mL) to yield the title compound as a reddish brown solid (467 mg, 38%).

### 4-{2-methanesulfonyl-7-oxo-5-[2-(triisopropylsilyl)ethynyl]pyrido[2,3-d]pyrimidin-8-yl}pyrrolidin-2-one

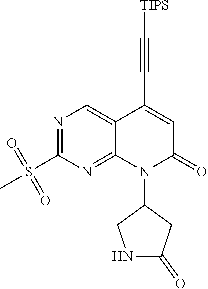

General procedure 10 was applied to 4-[2-(methylsulfanyl)-7-oxo-5-[2-(triisopropylsilyl)ethynyl]pyrido[2,3-d]pyrimidin-8-yl]pyrrolidin-2-one (420 mg, 0.920 mmol) and m-CPBA (349 mg, 2.02 mmol) in dichloromethane (6.0 mL) to yield the title compound as a yellow solid (278 mg, 62%).

### 4-(2-{[4-(4-methylpiperazin-1-yl)phenyl]amino}-7-oxo-5-[2-(triisopropylsilyl)ethynyl]pyrido [2,3-d]pyrimidin-8-yl)pyrrolidin-2-one

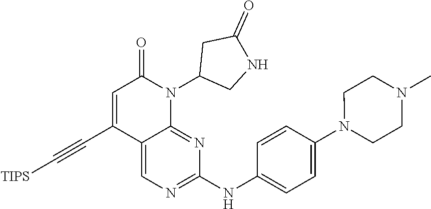

General procedure 13 was applied to 4-{2-methanesulfonyl-7-oxo-5-[2-(triisopropylsilyl)ethynyl]pyrido[2,3-d]pyrimidin-8-yl}pyrrolidin-2-one (284 mg, 0.581 mmol), 4-(4-methylpiperazin-1-yl)aniline (167 mg, 0.871 mmol) and trifluoroacetic acid (90.0 μL, 1.16 mmol) in 2-butanol (5.0 mL). The reaction was stirred overnight at 100° C. The residue was purified by reverse phase flash column chromatography eluting with acetonitrile (10-80%) in water (0.1% formic acid) to yield the title compound as a yellow solid (186 mg, 53%).

### 4-(5-ethynyl-2-{[4-(4-methylpiperazin-1-yl)phenyl]amino}-7-oxopyrido[2,3-d]pyrimidin-8-yl)pyrrolidin-2-one

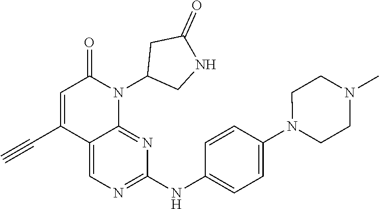 [SMILES: C#Cc1cc(=O)n(C2CNC(=O)C2)c2nc(Nc3ccc(N4CCN(C)CC4)cc3)ncc12]

General procedure 15 was applied to 4-(2-{[4-(4-methylpiperazin-1-yl)phenyl]amino}-7-oxo-5-[2-(triisopropylsilyl)ethynyl]pyrido[2,3-d]pyrimidin-8-yl)pyrrolidin-2-one (208 mg, 0.347 mmol) and potassium fluoride (201 mg, 3.47 mmol) in THF (2.0 mL), DMF (2.0 mL) and water (0.50 mL). The reaction was stirred for 1 h at 60° C. before being purified by reverse phase flash column chromatography eluting with acetonitrile (10-60%) in water (0.1% formic acid) to yield the title compound as a yellow solid (58.0 mg, 38%).

(ES, m/z): [M+H]⁺=444

¹H NMR (400 MHz, DMSO-d₆) δ 9.94 (s, 1H), 9.13 (s, 1H), 7.75 (d, J=47.7 Hz, 3H), 6.97-6.84 (m, 3H), 5.79 (s, 1H), 5.06 (s, 1H), 3.77 (dd, J=11.6, 5.5 Hz, 1H), 3.36 (d, J=11.5 Hz, 1H), 3.10 (t, J=5.0 Hz, 4H), 2.79 (dd, J=17.6, 6.8 Hz, 1H), 2.48 (t, J=5.0 Hz, 4H), 2.29 (d, J=2.1 Hz, 1H), 2.24 (s, 3H).

### Example 109

### tert-butyl 5-(hydroxymethyl)pyrazole-1-carboxylate

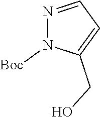 [SMILES: CC(C)(C)OC(=O)n1nccc1CO]

A mixture of 2H-pyrazol-3-ylmethanol (3.20 g, 32.6 mmol, 1.0 eq.) and triethylamine (8.25 g, 81.5 mmol, 2.5 eq.) in tetrahydrofuran (31.0 mL) was stirred for 5 min at 0° C. then di-tert-butyl dicarbonate (8.54 g, 39.1 mmol, 1.2 eq.) in dimethylformamide (32 mL) was added 10 min at 0° C. The resulting solution was stirred overnight at room temperature then diluted with water (100 mL), extracted with ethyl acetate (2×100 mL) and the combined organic layers concentrated under reduced pressure. The residue was purified by flash column chromatography, eluting with ethyl acetate (50%) in 40-60 petroleum ether to afford the title compound as a colourless oil (1.60 g, 25%).

### Ethyl 4-amino-2-(methylsulfanyl)pyrimidine-5-carboxylate

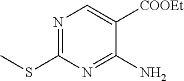

A solution of ethyl 4-chloro-2-(methylsulfanyl)pyrimidine-5-carboxylate (200 g, 859 mmol, 1.0 eq.) in ammonium hydroxide (400 mL) was treated with ethanol (600 mL) overnight at room temperature. The precipitated solids were collected by filtration, and washed with ethanol (3×10 mL). The resulting mixture was concentrated under reduced pressure to yield the title compound as a white solid (160 g, 87%).

### Ethyl 2-(methylsulfanyl)-4-propanamidopyrimidine-5-carboxylate

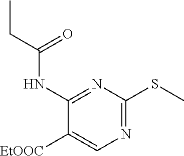 [SMILES: CCOC(=O)c1cnc(SC)nc1NC(=O)CC]

A solution of ethyl 4-amino-2-(methylsulfanyl)pyrimidine-5-carboxylate (19.0 g, 89.1 mmol, 1.0 eq.) and propionic anhydride (95 mL) was stirred overnight at 125° C. The mixture was allowed to cool to 15° C. The precipitated solids were collected by filtration, and washed with ethanol (3×30 mL) to yield the title compound as a pink solid (12.4 g, >100%).

### 5-hydroxy-6-methyl-2-(methylsulfanyl)-8H-pyrido[2,3-d]pyrimidin-7-one

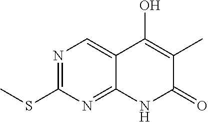

To a stirred solution of ethyl 2-(methylsulfanyl)-4-propanamidopyrimidine-5-carboxylate (10.3 g, 38.2 mmol, 1.0 eq.) in THF (62.0 mL) was added KHMDS (108 mL, 477 mmol, 12.5 eq.) dropwise at 0-10° C. over 10 minutes. The reaction was quenched with water (50 ml) at 0-10° C., concentrated under reduced pressure and then acidified to pH 3-4 with 4M aqueous hydrochloric acid. The precipitated solids were collected by filtration, and washed with water (3×5 mL). The resulting solids were dried under infrared light to yield the title compound as a white solid (7.25 g, 85%).

### 6-methyl-2-(methylsulfanyl)-7-oxo-8H-pyrido[2,3-d]pyrimidin-5-yl trifluoromethanesulfonate

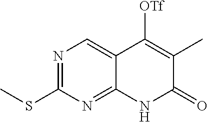

To a stirred solution of 5-hydroxy-6-methyl-2-(methylsulfanyl)-8H-pyrido[2,3-d]pyrimidin-7-one (7.00 g, 31.4 mmol, 1.0 eq.) in THF (70 mL) was added triethylamine (6.35 g, 62.7 mmol, 2.0 eq.). After stirring for 1 h at room temperature 1,1,1-trifluoro-N-phenyl-N-trifluoromethanesulfonylmethanesulfonamide (16.8 g, 47.0 mmol, 1.5 eq.) was added and the mixture stirred for 40 minutes then quenched with water (3.0 mL) and extracted with dichloromethane (3×20 mL). The combined organic layers were washed with brine (3×5 mL) and concentrated under reduced pressure. The residue was purified by trituration with ethyl acetate (20 mL). The precipitated solids were collected by filtration, washing with ethyl acetate (3×5 mL) to yield the title compound as a white solid (5.03 g, 45%).

### 6-methyl-2-(methylsulfanyl)-5-[2-(triisopropylsilyl)ethynyl]-8H-pyrido[2,3-d]pyrimidin-7-one

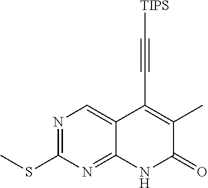

General procedure 9 was applied to 6-methyl-2-(methylsulfanyl)-7-oxo-8H-pyrido[2,3-d]pyrimidin-5-yl trifluoromethanesulfonate (4.00 g, 11.3 mmol), triisopropylsilylacetylene (3.08 g, 16.9 mmol), bis(triphenylphosphine)palladium(II) dichloride (395 mg, 0.563 mmol) and copper(I) iodide (3.22 g, 16.9 mmol) in ethyl acetate (40 mL) and triethylamine (6.84 g, 67.5 mmol). The residue was purified by trituration with ethyl acetate (50 ml). The precipitated solids were collected by filtration, washing with ethyl acetate (3×10 mL) to yield the title compound as a white solid (1.80 g, 41%).

### tert-butyl 5-{[6-methyl-2-(methylsulfanyl)-7-oxo-5-[2-(triisopropylsilyl)ethynyl]pyrido[2,3-d]pyrimidin-8-yl]methyl}pyrazole-1-carboxylate

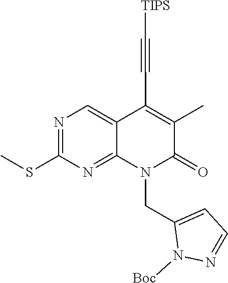

General procedure 18 was applied to 6-methyl-2-(methylsulfanyl)-5-[2-(triisopropylsilyl)ethynyl]-8H-pyrido[2,3-d]pyrimidin-7-one (520 mg, 1.34 mmol), tert-butyl 5-(hydroxymethyl)pyrazole-1-carboxylate (292 mg, 1.47 mmol), DIAD (542 mg, 2.68 mmol) and triphenylphosphine (704 mg, 2.68 mmol) in dichloromethane (10 mL). The residue was purified by flash column chromatography, eluting with ethyl acetate (10%) in 40-60 petroleum ether to afford the title compound as a yellow solid (230 mg, 30%).

### tert-butyl 5-({2-methanesulfonyl-6-methyl-7-oxo-5-[2-(triisopropylsilyl)ethynyl]pyrido[2,3-d]pyrimidin-8-yl}methyl)pyrazole-1-carboxylate

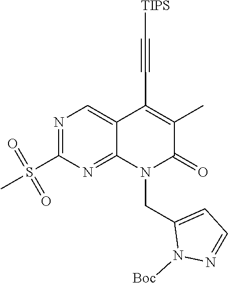

General procedure 10 was applied to tert-butyl 5-{[6-methyl-2-(methylsulfanyl)-7-oxo-5-[2-(triisopropylsilyl)ethynyl]pyrido [2,3-d]pyrimidin-8-yl]methyl}pyrazole-1-carboxylate (400 mg, 0.704 mmol) and m-CPBA (267 mg, 1.55 mmol) in dichloromethane (6.0 mL) to yield the title compound as a light yellow oil (382 mg, 90%).

### tert-butyl 5-[(6-methyl-2-{[4-(4-methylpiperazin-1-yl)phenyl]amino}-7-oxo-5-[2-(triisopropylsilyl)ethynyl]pyrido[2,3-d]pyrimidin-8-yl)methyl]pyrazole-1-carboxylate

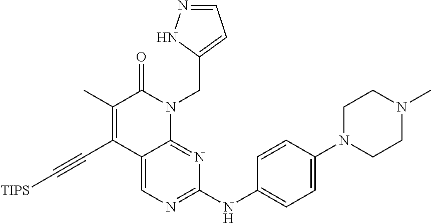

General procedure 13 was applied to tert-butyl 5-({2-methanesulfonyl-6-methyl-7-oxo-5-[2-(triisopropylsilyl)ethynyl]pyrido [2,3-d]pyrimidin-8-yl}methyl)pyrazole-1-carboxylate (287 mg, 0.478 mmol), 4-(4-methylpiperazin-1-yl)aniline (137 mg, 0.717 mmol) and trifluoroacetic acid (70.0 μL, 0.956 mmol) in 2-butanol (5.0 mL). The reaction was stirred overnight at 100° C. before being purified by reverse phase flash column chromatography eluting with acetonitrile (10-50%) in water (0.1% formic acid) to yield the title compound as a yellow solid (150 mg, 44%).

### 5-ethynyl-6-methyl-2-{[4-(4-methylpiperazin-1-yl)phenyl]amino}-8-(2H-pyrazol-3-ylmethyl)pyrido[2,3-d]pyrimidin-7-one

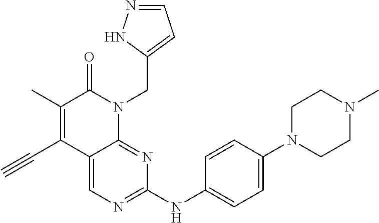 [SMILES: C#Cc1c(C)c(=O)n(Cc2ccn[nH]2)c2nc(Nc3ccc(N4CCN(C)CC4)cc3)ncc12]

General procedure 15 was applied to 6-methyl-2-{[4-(4-methylpiperazin-1-yl)phenyl]amino}-8-(2H-pyrazol-3-ylmethyl)-5-[2-(triisopropylsilyl)ethynyl]pyrido[2,3-d]pyrimidin-7-one (120 mg, 0.196 mmol) and potassium fluoride (114 mg, 1.96 mmol) in THF (2.0 mL), DMF (2.0 mL) and water (0.50 mL). The reaction was stirred for 1 h at 60° C. The residue was purified by reverse phase flash column chromatography eluting with acetonitrile (10-80%) in water (0.1% formic acid) to yield the title compound as a yellow solid (19.0 mg, 21%).

(ES, m/z): [M+H]⁺=455

¹H-NMR (300 MHz, DMSO-d₆) δ 9.91 (s, 1H), 8.75 (s, 1H), 8.20 (s, 1H), 7.61-7.49 (m, 3H), 6.85 (d, J=8.7 Hz, 2H), 6.09 (d, J=2.1 Hz, 1H), 5.48 (s, 2H), 5.22 (s, 1H), 3.08 (t, J=4.9 Hz, 4H), 2.47 (d, J=4.8 Hz, 4H), 2.25 (s, 6H).

### Example 110

### 6-methyl-8-[(1-methylpyrazol-3-yl)methyl]-2-(methylsulfanyl)-5-[2-(triisopropylsilyl)ethynyl]pyrido[2,3-d]pyrimidin-7-one

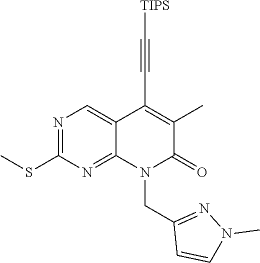

General procedure 18 was applied to 6-methyl-2-(methylsulfanyl)-5-[2-(triisopropylsilyl)ethynyl]-8H-pyrido[2,3-d]pyrimidin-7-one (700 mg, 1.81 mmol), (1-methylpyrazol-3-yl)methanol (223 mg, 1.99 mmol), DIAD (730 mg, 3.61 mmol) and triphenylphosphine (947 mg, 3.61 mmol) in dichloromethane (10 mL). The residue was purified by flash column chromatography, eluting with ethyl acetate (10%) in 40-60 petroleum ether to afford the title compound as a yellow solid (600 mg, 69%).

(ES, m/z): [M+H]⁺=482

### 2-methanesulfonyl-6-methyl-8-[(1-methylpyrazol-3-yl)methyl]-5-[2-(triisopropylsilyl)ethynyl]pyrido[2,3-d]pyrimidin-7-one

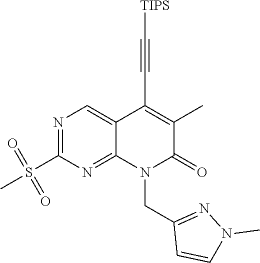

General procedure 10 was applied to 6-methyl-8-[(1-methylpyrazol-3-yl)methyl]-2-(methylsulfanyl)-5-[2-(triisopropylsilyl)ethynyl]pyrido[2,3-d]pyrimidin-7-one (487 mg, 1.01 mmol) and m-CPBA (384 mg, 2.22 mmol) in dichloromethane (10 mL) to yield the title compound as a light yellow solid (450 mg, 87%).

(ES, m/z): [M+H]⁺=514

### 6-methyl-2-{[4-(4-methylpiperazin-1-yl)phenyl]amino}-8-[(1-methylpyrazol-3-yl)methyl]-5-[2-(triisopropylsilyl)ethynyl]pyrido[2,3-d]pyrimidin-7-one

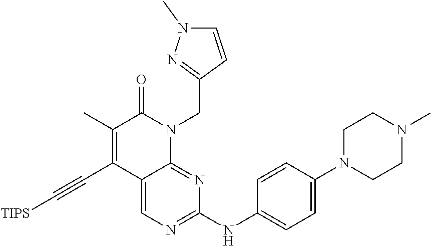

General procedure 13 was applied to 2-methanesulfonyl-6-methyl-8-[(1-methylpyrazol-3-yl)methyl]-5-[2-(triisopropylsilyl)ethynyl]pyrido[2,3-d]pyrimidin-7-one (82.0 mg, 0.160 mmol), 4-(4-methylpiperazin-1-yl)aniline (45.8 mg, 0.240 mmol) and trifluoroacetic acid (20.0 μL, 0.320 mmol) in 2-butanol (2.0 mL). The reaction was stirred for 2 h at 100° C. The residue was purified by reverse phase flash column chromatography eluting with acetonitrile (20-100%) in water (0.1% formic acid) to yield the title compound as a light yellow solid (59.0 mg, 59%).

(ES, m/z): [M+H]⁺=625

### 5-ethynyl-6-methyl-2-{[4-(4-methylpiperazin-1-yl)phenyl]amino}-8-[(1-methylpyrazol-3-yl)methyl]pyrido[2,3-d]pyrimidin-7-one

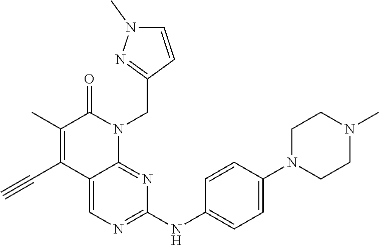 [SMILES: C#Cc1c(C)c(=O)n(Cc2ccn(C)n2)c2nc(Nc3ccc(N4CCN(C)CC4)cc3)ncc12]

General procedure 15 was applied to 6-methyl-2-{[4-(4-methylpiperazin-1-yl)phenyl]amino}-8-[(1-methylpyrazol-3-yl)methyl]-5-[2-(triisopropylsilyl)ethynyl]pyrido[2,3-d]pyrimidin-7-one (150 mg, 0.240 mmol) and potassium fluoride (139 mg, 2.40 mmol) in THF (1.5 mL), DMF (1.5 mL) and water (150 μL). The reaction was stirred for 2 h at 60° C. before being purified by reverse phase flash column chromatography eluting with acetonitrile (10-50%) in water (0.1% formic acid) to yield the title compound as a yellow solid (72.6 mg, 65%).

(ES, m/z): [M+H]⁺=469.

¹H NMR (400 MHz, DMSO-d₆) δ 9.92 (s, 1H), 8.75 (s, 1H), 8.14 (s, 1H), 7.61 (d, J=8.5 Hz, 2H), 7.53 (d, J=2.2 Hz, 1H), 6.86 (d, J=8.9 Hz, 2H), 6.04 (d, J=2.2 Hz, 1H), 5.43 (s, 2H), 5.23 (s, 1H), 3.73 (s, 3H), 3.09 (t, J=5.1 Hz, 4H), 2.25 (d, J=9.8 Hz, 6H).

### Example 111

### tert-butyl 4-(hydroxymethyl)imidazole-1-carboxylate

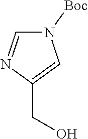 [SMILES: CC(C)(C)OC(=O)n1cnc(CO)c1]

A solution of 4-(hydroxymethyl)imidazole (1.26 g, 12.8 mmol, 1.0 eq.), triethylamine (3.25 g, 32.1 mmol, 2.5 eq.) and boc-anhydride (3.36 g, 15.4 mmol, 1.2 eq.) in THF (5.0 mL) and DMF (9.9 mL) was prepared at 0° C. The resulting mixture was stirred at room temperature overnight then extracted with diethyl ether (2×25 mL). The combined organic layers were washed with water (200 mL) and brine (200 mL), dried (Na₂SO₄) and concentrated under reduced pressure. The residue was purified by flash column chromatography, eluting with ethyl acetate (50%) in 40-60 petroleum ether to afford the title compound as a yellow oil (760 mg, 30%).

### tert-butyl 5-{[6-methyl-2-(methylsulfanyl)-7-oxo-5-[2-(triisopropylsilyl) ethynyl]pyrido[2,3-d]pyrimidin-8-yl]methyl}imidazole-1-carboxylate

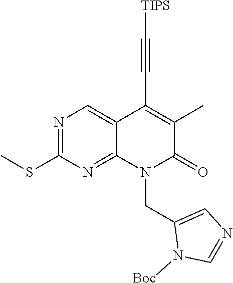

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), tert-butyl 4-(hydroxymethyl)imidazole-1-carboxylate (562 mg, 2.84 mmol), triphenylphosphine (2.71 g, 10.3 mmol) and DIAD (2.09 g, 10.3 mmol) in dichloromethane (10 mL). The residue was purified by flash column chromatography, eluting with ethyl acetate (10%) in 40-60 petroleum ether to afford the title compound as a brown oil (1.34 g, 91%).

### tert-butyl 5-({2-methanesulfonyl-6-methyl-7-oxo-5-[2-(triisopropylsilyl)ethynyl]pyrido[2,3-d]pyrimidin-8-yl}methyl)imidazole-1-carboxylate

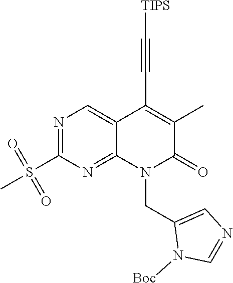

General procedure 10 was applied to tert-butyl 5-{[6-methyl-2-(methylsulfanyl)-7-oxo-5-[2-(triisopropylsilyl)ethynyl]pyrido[2,3-d]pyrimidin-8-yl]methyl}imidazole-1-carboxylate (367 mg, 0.646 mmol) and m-CPBA (245 mg, 1.42 mmol) in dichloromethane (10 mL) to yield the title compound as a light yellow oil (342 mg, 88%).

### tert-butyl 5-[(6-methyl-2-{[4-(4-methylpiperazin-1-yl)phenyl]amino}-7-oxo-5-[2-(triisopropylsilyl)ethynyl]pyrido[2,3-d]pyrimidin-8-yl)methyl]imidazole-1-carboxylate

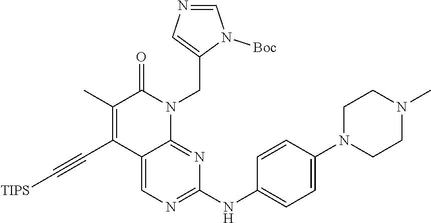

General procedure 13 was applied to tert-butyl 5-({2-methanesulfonyl-6-methyl-7-oxo-5-[2-(triisopropylsilyl)ethynyl]pyrido[2,3-d]pyrimidin-8-yl}methyl)imidazole-1-carboxylate (205 mg, 0.342 mmol), 4-(4-methylpiperazin-1-yl)aniline (98.1 mg, 0.513 mmol) and trifluoroacetic acid (77.9 mg, 0.684 mmol) in 2-butanol (3.0 mL). The reaction was stirred for 2.5 h at 80° C. The residue was purified by reverse phase flash column chromatography eluting with acetonitrile (10-100%) in water (0.1% formic acid) to yield the title compound as a red solid (82.0 mg, 34%).

### 8-(3H-imidazol-4-ylmethyl)-6-methyl-2-{[4-(4-methylpiperazin-1-yl)phenyl]amino}-5-[2-(triisopropylsilyl)ethynyl]pyrido[2,3-d]pyrimidin-7-one

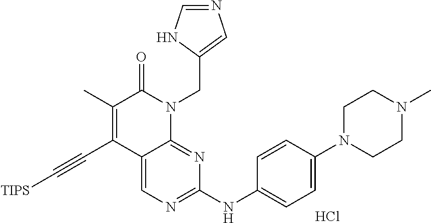

A solution of tert-butyl 5-[(6-methyl-2-{[4-(4-methylpiperazin-1-yl)phenyl]amino}-7-oxo-5-[2-(triisopropylsilyl)ethynyl]pyrido[2,3-d]pyrimidin-8-yl)methyl]imidazole-1-carboxylate (80.0 mg, 0.113 mmol, 1.0 eq.) in 4M hydrogen chloride in 1,4-dioxane (70.0 μL, 2.26 mmol, 20 eq.) in dichloromethane (2.0 mL) was stirred for 1 h at room temperature. The resulting mixture was concentrated under reduced pressure to yield the title compound as a red solid (53.0 mg, 77%).

### 5-ethynyl-8-(3H-imidazol-4-ylmethyl)-6-methyl-2-{[4-(4-methylpiperazin-1-yl)phenyl]amino}pyrido[2,3-d]pyrimidin-7-one

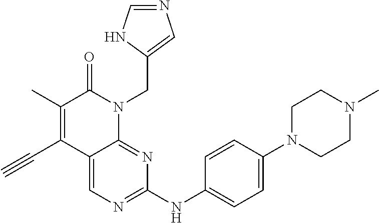 [SMILES: C#Cc1c(C)c(=O)n(Cc2cnc[nH]2)c2nc(Nc3ccc(N4CCN(C)CC4)cc3)ncc12]

General procedure 15 was applied to 8-(3H-imidazol-4-ylmethyl)-6-methyl-2-{[4-(4-methylpiperazin-1-yl)phenyl]amino}-5-[2-(triisopropylsilyl)ethynyl]pyrido[2,3-d]pyrimidin-7-one (52.0 mg, 0.0851 mmol) and potassium fluoride (49.5 mg, 0.850 mmol) in THF (1.5 mL), DMF (1.5 mL) and water (0.20 mL). The reaction was stirred for 1 h at 60° C. before being purified by reverse phase flash column chromatography eluting with acetonitrile (10-60%) in water (0.1% formic acid) to yield the title compound as a yellow solid (6.00 mg, 15%).

(ES, m/z): [M+H]⁺=455

¹H NMR (400 MHz, DMSO-d₆) δ 9.90 (s, 1H), 8.75 (s, 1H), 7.59 (d, J=8.7 Hz, 2H), 7.49 (s, 1H), 6.90-6.80 (m, 3H), 5.40 (s, 2H), 5.21 (s, 1H), 3.09 (t, J=5.0 Hz, 4H), 2.24 (d, J=7.7 Hz, 6H).

### Example 112

### 6-methyl-8-[(2-methylpyrazol-3-yl)methyl]-2-(methylsulfanyl)-5-[2-(triisopropylsilyl)ethynyl]pyrido[2,3-d]pyrimidin-7-one

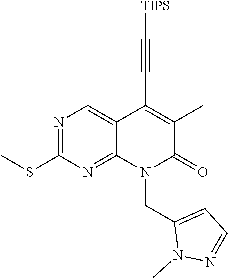

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), (2-methylpyrazol-3-yl)methanol (318 mg, 2.84 mmol), triphenylphosphine (1.35 g, 5.16 mmol) and DIAD (1.04 g, 5.16 mmol) in dichloromethane (20 mL). The residue was purified by reverse phase flash column chromatography eluting with acetonitrile (95-100%) in water (0.4% formic acid) (0.4%) to afford the title compound as a yellow oil (1.02 g, 82%).

(ES, m/z): [M+H]⁺=482.2

### 2-methanesulfonyl-6-methyl-8-[(2-methylpyrazol-3-yl)methyl]-5-[2-(triisopropylsilyl)ethynyl]pyrido[2,3-d]pyrimidin-7-one

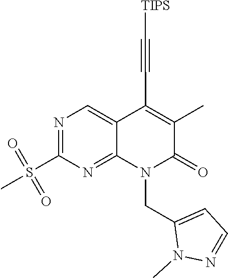

General procedure 10 was applied to 6-methyl-8-[(2-methylpyrazol-3-yl)methyl]-2-(methylsulfanyl)-5-[2-(triisopropylsilyl)ethynyl]pyrido[2,3-d]pyrimidin-7-one (900 mg, 1.87 mmol) and m-CPBA (709 mg, 4.110 mmol) in dichloromethane (10 mL) to afford the title compound as a yellow solid (950 mg, 99%), which was used in the next step without further purification.

(ES, m/z): [M+H]1=514.2

### 6-methyl-2-{[4-(4-methylpiperazin-1-yl)phenyl]amino}-8-[(2-methylpyrazol-3-yl)methyl]-5-[2-(triisopropylsilyl)ethynyl]pyrido[2,3-d]pyrimidin-7-one

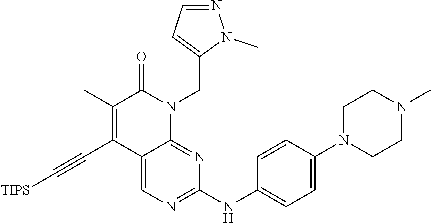

General procedure 13 was applied to 2-methanesulfonyl-6-methyl-8-[(2-methylpyrazol-3-yl)methyl]-5-[2-(triisopropylsilyl) ethynyl]pyrido[2,3-d]pyrimidin-7-one (400 mg, 0.779 mmol), 4-(4-methylpiperazin-1-yl)aniline (223 mg, 1.17 mmol) and trifluoroacetic acid (177 mg, 1.56 mmol) in 2-butanol (2.0 mL). The reaction was stirred for 12 h at 100° C.

The residue was purified by reverse phase flash column chromatography eluting with acetonitrile (50-60%) in water (0.4% trifluoroacetic acid) to afford the title compound as an orange solid (200 mg, 41%).

(ES, m/z): [M+H]⁺=625.4

### 5-ethynyl-6-methyl-2-{[4-(4-methylpiperazin-1-yl)phenyl]amino}-8-[(2-methylpyrazol-3-yl)methyl]pyrido[2,3-d]pyrimidin-7-one

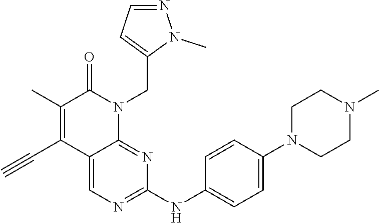 [SMILES: C#Cc1c(C)c(=O)n(Cc2ccnn2C)c2nc(Nc3ccc(N4CCN(C)CC4)cc3)ncc12]

General procedure 15 was applied to 6-methyl-2-{[4-(4-methylpiperazin-1-yl)phenyl]amino}-8-[(2-methylpyrazol-3-yl)methyl]-5-[2-(triisopropylsilyl)ethynyl]pyrido[2,3-d]pyrimidin-7-one (170 mg, 0.272 mmol) and potassium fluoride (158 mg, 2.72 mmol) in DMF (1.0 mL), THF (1.0 mL) and water (0.2.0 mL). The reaction was stirred for 2 h at 65° C. before being purified by reverse phase flash column chromatography eluting with acetonitrile (30-35%) in water (0.4% formic acid) to afford as a yellow solid (7.40 mg, 5%).

(ES, m/z): [M+H]⁺=469.2

¹H NMR (400 MHz, DMSO-d₆) δ 9.92 (s, 1H), 8.77 (s, 1H), 7.38 (s, 2H), 7.22 (d, J=1.9 Hz, 1H), 6.84 (d, J=8.4 Hz, 2H), 5.87 (s, 1H), 5.45 (s, 2H), 5.25 (s, 1H), 3.89 (s, 3H), 3.07 (t, J=5.0 Hz, 4H), 2.45 (t, J=5.0 Hz, 4H), 2.23 (d, J=8.1 Hz, 6H).

### Example 113

### 6-methyl-2-(methylsulfanyl)-8-(1,2-oxazol-5-ylmethyl)-5-[2-(triisopropylsilyl)ethynyl]pyrido[2,3-d]pyrimidin-7-one

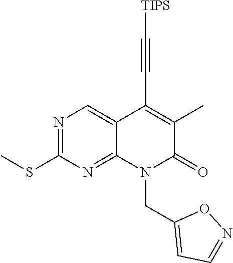

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), 1,2-oxazol-5-ylmethanol (280 mg, 2.84 mmol), triphenylphosphine (1.35 g, 5.16 mmol) and DIAD (1.04 g, 5.16 mmol) in dichloromethane (20 mL). The residue was purified by flash column chromatography, eluting with ethyl acetate (10%) in 40-60 petroleum ether to afford the title compound as a white solid (400 mg, 33%). 2-methanesulfonyl-6-methyl-8-(1,2-oxazol-5-ylmethyl)-5-[2-(triisopropylsilyl)ethynyl]pyrido[2,3-d]pyrimidin-7-one

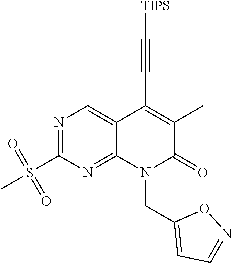

General procedure 10 was applied to 6-methyl-2-(methylsulfanyl)-8-(1,2-oxazol-5-ylmethyl)-5-[2-(triisopropylsilyl)ethynyl]pyrido[2,3-d]pyrimidin-7-one (382 mg, 0.815 mmol) and m-CPBA (309 mg, 1.79 mmol) in dichloromethane (6.0 mL) to yield the title compound as a yellow oil (286 mg, 70%).

### 6-methyl-2-{[4-(4-methylpiperazin-1-yl)phenyl]amino}-8-(1,2-oxazol-5-ylmethyl)-5-[2-(triisopropylsilyl)ethynyl]pyrido[2,3-d]pyrimidin-7-one

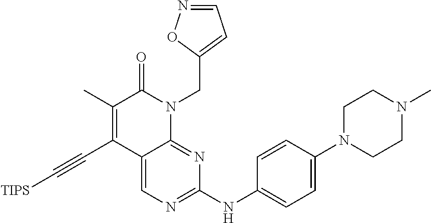

General procedure 13 was applied to 2-methanesulfonyl-6-methyl-8-(1,2-oxazol-5-ylmethyl)-5-[2-(triisopropylsilyl)ethynyl]pyrido[2,3-d]pyrimidin-7-one (286 mg, 0.571 mmol), 4-(4-methylpiperazin-1-yl)aniline (164 mg, 0.856 mmol) and trifluoroacetic acid (0.08 mL, 1.14 mmol) in 2-butanol (3.0 mL). The reaction was stirred overnight at 100° C. before being purified by reverse phase flash column chromatography eluting with acetonitrile (10-80%) in water (0.1% formic acid) to yield the title compound as a reddish brown yellow oil (205 mg, 59%).

### 5-ethynyl-6-methyl-2-{[4-(4-methylpiperazin-1-yl)phenyl]amino}-8-(1,2-oxazol-5-ylmethyl)pyrido[2,3-d]pyrimidin-7-one

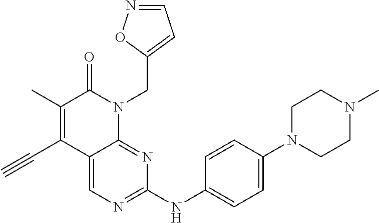 [SMILES: C#Cc1c(C)c(=O)n(Cc2ccno2)c2nc(Nc3ccc(N4CCN(C)CC4)cc3)ncc12]

General procedure 15 was applied to 6-methyl-2-{[4-(4-methylpiperazin-1-yl)phenyl]amino}-8-(1,2-oxazol-5-ylmethyl)-5-[2-(triisopropylsilyl)ethynyl]pyrido[2,3-d]pyrimidin-7-one (250 mg, 0.409 mmol) and potassium fluoride (237 mg, 4.09 mmol) in THF (2.0 mL), DMF (2.0 mL) and water (0.50 mL). The reaction was stirred for 1 h at 60° C. before being purified by reverse phase flash column chromatography eluting with acetonitrile (10-50%) in water (0.1% formic acid) to yield the title compound as a yellow solid (60.0 mg, 32%).

(ES, m/z): [M+H]⁺=456

¹H NMR (400 MHz, DMSO-d₆) δ 10.00 (s, 1H), 8.77 (s, 1H), 8.49 (d, J=1.8 Hz, 1H), 7.46 (d, J=8.5 Hz, 2H), 6.86 (d, J=8.7 Hz, 2H), 6.34 (s, 1H), 5.59 (s, 2H), 5.27 (s, 1H), 3.08 (t, J=5.0 Hz, 4H), 2.48 (d, J=5.0 Hz, 4H), 2.24 (d, J=1.8 Hz, 6H).

### Example 114

### 6-methyl-2-(methylsulfanyl)-8-(1,3-oxazol-2-ylmethyl)-5-[2-(triisopropylsilyl)ethynyl]pyrido[2,3-d]pyrimidin-7-one

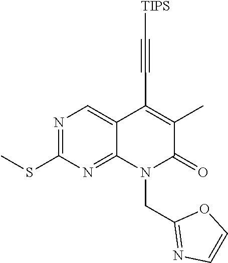

General procedure 18 was applied to 6-methyl-2-(methylsulfanyl)-5-[2-(triisopropylsilyl)ethynyl]-8H-pyrido[2,3-d]pyrimidin-7-one (530 mg, 1.37 mmol), 1,3-oxazol-2-ylmethanol (149 mg, 1.50 mmol), triphenylphosphine (717 mg, 2.73 mmol) and DIAD (553 mg, 2.73 mmol) in dichloromethane (8.0 mL). The residue was purified by flash column chromatography, eluting with ethyl acetate (8%) in 40-60 petroleum ether to afford the title compound as a light yellow oil (521 mg, 81%).

### 2-methanesulfonyl-6-methyl-8-(1,3-oxazol-2-ylmethyl)-5-[2-(triisopropylsilyl)ethynyl]pyrido[2,3-d]pyrimidin-7-one

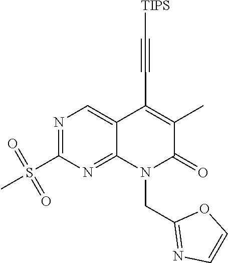

General procedure 10 was applied to 6-methyl-2-(methylsulfanyl)-8-(1,3-oxazol-2-ylmethyl)-5-[2-(triisopropylsilyl)ethynyl]pyrido[2,3-d]pyrimidin-7-one (450 mg, 0.960 mmol) and m-CPBA (364 mg, 2.11 mmol) in dichloromethane (8.0 mL) to yield the title compound as a light yellow oil (420 mg, 87%).

### 6-methyl-2-{[4-(4-methylpiperazin-1-yl)phenyl]amino}-8-(1,3-oxazol-2-ylmethyl)-5-[2-(triisopropylsilyl)ethynyl]pyrido[2,3-d]pyrimidin-7-one

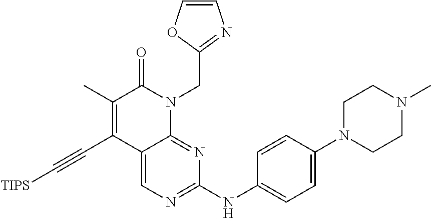

General procedure 13 was applied to 2-methanesulfonyl-6-methyl-8-(1,3-oxazol-2-ylmethyl)-5-[2-(triisopropylsilyl)ethynyl]pyrido[2,3-d]pyrimidin-7-one (400 mg, 0.799 mmol), 4-(4-methylpiperazin-1-yl)aniline (183 mg, 0.959 mmol) and trifluoroacetic acid (0.120 mL, 1.59 mmol) in 2-butanol (6.0 mL). The reaction was stirred for 1 h at 80° C. The residue was purified by reverse phase flash column chromatography eluting with acetonitrile (10-80%) in water (0.1% formic acid) to yield the title compound as a light yellow solid (105 mg, 21%).

### 5-ethynyl-6-methyl-2-{[4-(4-methylpiperazin-1-yl)phenyl]amino}-8-(1,3-oxazol-2-ylmethyl)pyrido[2,3-d]pyrimidin-7-one

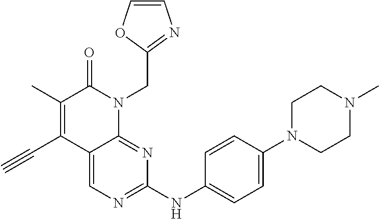 [SMILES: C#Cc1c(C)c(=O)n(Cc2ncco2)c2nc(Nc3ccc(N4CCN(C)CC4)cc3)ncc12]

General procedure 15 was applied to 6-methyl-2-{[4-(4-methylpiperazin-1-yl)phenyl]amino}-8-(1,3-oxazol-2-ylmethyl)-5-[2-(triisopropylsilyl)ethynyl]pyrido[2,3-d]pyrimidin-7-one (105 mg, 0.172 mmol) and potassium fluoride (99.70 mg, 1.72 mmol) in THF (2.0 mL), DMF (2.0 mL) and water (0.50 mL). The reaction was stirred for 1 h at 60° C. before being purified by reverse phase flash column chromatography eluting with acetonitrile (10-50%) in water (0.1% formic acid) to yield the title compound as a yellow solid (30 mg, 38%).

(ES, m/z): [M+H]⁺=456

¹H-NMR (300 MHz, DMSO-d₆) δ 9.99 (s, 1H), 8.79 (s, 1H), 8.10 (s, 1H), 7.38 (d, J=8.3 Hz, 2H), 7.13 (s, 1H), 6.83 (d, J=8.7 Hz, 2H), 5.55 (s, 2H), 5.29 (s, 1H), 3.09 (t, J=5.0 Hz, 4H), 2.26 (d, J=3.1 Hz, 6H).

### Example 115

### 6-methyl-2-(methylsulfanyl)-8-(pyridin-3-ylmethyl)-5-[2-(triisopropylsilyl)ethynyl]pyrido[2,3-d]pyrimidin-7-one

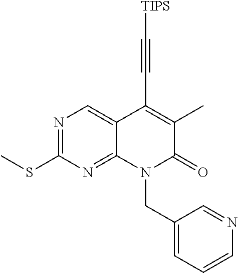

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), pyridin-3-ylmethanol (310 mg, 2.84 mmol), triphenylphosphine (1.35 g, 5.16 mmol) and DIAD (1.04 g, 5.16 mmol) in dichloromethane (20 mL). The residue was purified by reverse phase flash column chromatography eluting with acetonitrile (95-100%) in water (0.4% formic acid) to afford as a white solid (690 mg, 56%).

(ES, m/z): [M+H]⁺=479.2

### 2-methanesulfinyl-6-methyl-8-(pyridin-3-ylmethyl)-5-[2-(triisopropylsilyl)ethynyl]pyrido[2,3-d]pyrimidin-7-one

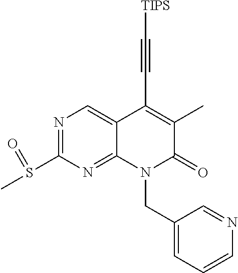

To a solution of 6-methyl-2-(methylsulfanyl)-8-(pyridin-3-ylmethyl)-5-[2-(triisopropylsilyl)ethynyl]pyrido[2,3-d]pyrimidin-7-one (510 mg, 1.06 mmol, 1.0 eq.) in dichloromethane (10 mL) was added 2-(benzenesulfonyl)-3-phenyloxaziridine (370 mg, 1.42 mmol, 1.3 eq.). The solution was stirred overnight at room temperature before being purified by flash column chromatography, eluting with ethyl acetate (90%) in 40-60 petroleum ether to afford the title compound as a yellow solid (300 mg, 57%).

(ES, m/z): [M+H]⁺=495.2

### 6-methyl-2-{[4-(4-methylpiperazin-1-yl)phenyl]amino}-8-(pyridin-3-ylmethyl)-5-[2-(triisopropylsilyl)ethynyl]pyrido[2,3-d]pyrimidin-7-one

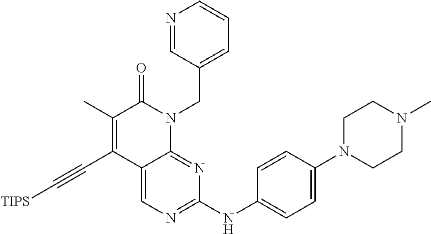

General procedure 13 was applied to 2-methanesulfinyl-6-methyl-8-(pyridin-3-ylmethyl)-5-[2-(triisopropylsilyl)ethynyl]pyrido[2,3-d]pyrimidin-7-one (300 mg, 0.606 mmol), 4-(4-methylpiperazin-1-yl)aniline (174 mg, 0.909 mmol) and trifluoroacetic acid (138 mg, 1.21 mmol) in 2-butanol (3.0 mL). The reaction was stirred overnight at 100° C. before being purified by reverse phase flash column chromatography eluting with acetonitrile (30-35%) in water (0.4% formic acid) to afford the title compound as an orange solid (236 mg, 63%).

(ES, m/z): [M+H]⁺=622.4

### 5-ethynyl-6-methyl-2-{[4-(4-methylpiperazin-1-yl)phenyl]amino}-8-(pyridin-3-ylmethyl)pyrido[2,3-d]pyrimidin-7-one

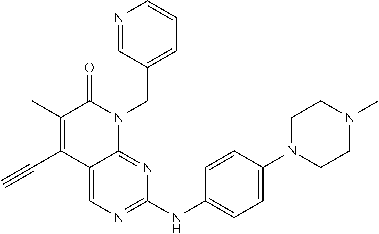 [SMILES: C#Cc1c(C)c(=O)n(Cc2cccnc2)c2nc(Nc3ccc(N4CCN(C)CC4)cc3)ncc12]

General procedure 15 was applied to 6-methyl-2-{[4-(4-methylpiperazin-1-yl)phenyl]amino}-8-(pyridin-3-ylmethyl)-5-[2-(triisopropylsilyl)ethynyl]pyrido[2,3-d]pyrimidin-7-one (200 mg, 0.322 mmol) and potassium fluoride (187 mg, 3.22 mmol) in DMF (1.0 mL), THF (1.0 mL) and water (0.2.0 mL). The reaction was stirred for 2 h at 60° C. before being purified by reverse phase flash column chromatography eluting with acetonitrile (20-30%) in water (0.4% formic acid) to afford the title compound as a yellow solid (118 mg, 77%).

(ES, m/z): [M+H]⁺=466.1.

¹H NMR (400 MHz, DMSO-d₆) δ 9.93 (s, 1H), 8.77 (s, 1H), 8.56 (s, 1H), 8.43 (dd, J=4.8, 1.6 Hz, 1H), 7.62 (s, 1H), 7.44 (d, J=8.4 Hz, 2H), 7.29 (dd, J=7.9, 4.8 Hz, 1H), 6.85 (d, J=8.6 Hz, 2H), 5.48 (s, 2H), 5.24 (s, 1H), 3.07 (t, J=5.0 Hz, 4H), 2.45 (t, J=5.0 Hz, 4H), 2.25 (s, 3H), 2.22 (s, 3H).

### Example 116

### 6-methyl-2-(methylsulfanyl)-8-(pyridin-3-ylmethyl)-5-[2-(triisopropylsilyl)ethynyl]pyrido[2,3-d]pyrimidin-7-one

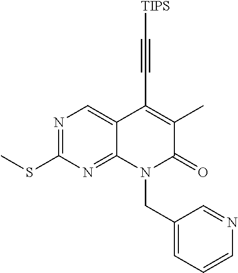

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), pyridin-3-ylmethanol (309 mg, 2.84 mmol), triphenylphosphine (1.35 g, 5.16 mmol) and DIAD (1.04 g, 5.16 mmol) in dichloromethane (20 mL). The residue was purified by reverse phase flash column chromatography eluting with acetonitrile (95-100%) in water (0.4% formic acid) to afford the title compound as a white solid (690 mg, 56%).

(ES, m/z): [M+H]⁺=479.2

### 2-methanesulfinyl-6-methyl-8-(pyridin-3-ylmethyl)-5-[2-(triisopropylsilyl)ethynyl]pyrido[2,3-d]pyrimidin-7-one

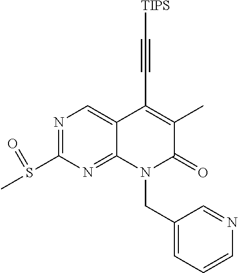

To a solution of 6-methyl-2-(methylsulfanyl)-8-(pyridin-3-ylmethyl)-5-[2-(triisopropylsilyl)ethynyl]pyrido[2,3-d]pyrimidin-7-one (510 mg, 1.06 mmol, 1.0 eq.) in dichloromethane (10 mL) was added 2-(benzenesulfonyl)-3-phenyloxaziridine (370 mg, 1.42 mmol, 1.3 eq.). The solution was stirred overnight at room temperature before being purified by flash column chromatography, eluting with ethyl acetate (90%) in 40-60 petroleum ether to afford the title compound as a yellow solid (300 mg, 57%).

(ES, m/z): [M+H]⁺=495.2

### 6-methyl-2-{[4-(4-methylpiperazin-1-yl)phenyl]amino}-8-(pyridin-3-ylmethyl)-5-[2-(triisopropylsilyl)ethynyl]pyrido[2,3-d]pyrimidin-7-one

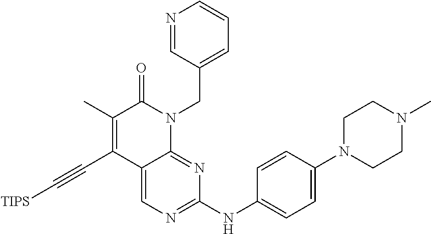

General procedure 13 was applied to 2-methanesulfinyl-6-methyl-8-(pyridin-3-ylmethyl)-5-[2-(triisopropylsilyl)ethynyl]pyrido[2,3-d]pyrimidin-7-one (300 mg, 0.606 mmol), 4-(4-methylpiperazin-1-yl)aniline (174 mg, 0.909 mmol) and trifluoroacetic acid (138 mg, 1.21 mmol) in 2-butanol (3.0 mL). The reaction was stirred overnight at 100° C. The residue was purified by reverse phase flash column chromatography eluting with acetonitrile (30-35%) in water (0.4% formic acid) to afford the title compound as an orange solid (236 mg, 63%).

(ES, m/z): [M+H]⁺=622.4

### 5-ethynyl-6-methyl-2-{[4-(4-methylpiperazin-1-yl)phenyl]amino}-8-(pyridin-3-ylmethyl)pyrido[2,3-d]pyrimidin-7-one

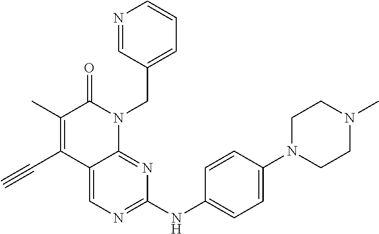 [SMILES: C#Cc1c(C)c(=O)n(Cc2cccnc2)c2nc(Nc3ccc(N4CCN(C)CC4)cc3)ncc12]

General procedure 15 was applied to 6-methyl-2-{[4-(4-methylpiperazin-1-yl)phenyl]amino}-8-(pyridin-3-ylmethyl)-5-[2-(triisopropylsilyl)ethynyl]pyrido[2,3-d]pyrimidin-7-one (200 mg, 0.322 mmol) and potassium fluoride (187 mg, 3.22 mmol) in DMF (1.0 mL), THF (1.0 mL) and water (0.20 mL). The reaction was stirred for 2 h at 60° C. before being purified by reverse phase flash column chromatography eluting with acetonitrile (20-30%) in water (0.4% formic acid) to afford the title compound as a yellow solid (118 mg, 77%).

(ES, m/z): [M+H]⁺=466.1.

¹H NMR (400 MHz, DMSO-d₆) δ 9.93 (s, 1H), 8.77 (s, 1H), 8.56 (s, 1H), 8.43 (dd, J=4.8, 1.6 Hz, 1H), 7.62 (s, 1H), 7.44 (d, J=8.4 Hz, 2H), 7.29 (dd, J=7.9, 4.8 Hz, 1H), 6.85 (d, J=8.6 Hz, 2H), 5.48 (s, 2H), 5.24 (s, 1H), 3.07 (t, J=5.0 Hz, 4H), 2.45 (t, J=5.0 Hz, 4H), 2.25 (s, 3H), 2.22 (s, 3H).

### Example 117

### 4-[(tert-butyldimethylsilyl)oxy]pyrrolidin-2-one

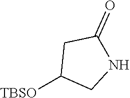 [SMILES: CC(C)(C)[Si](C)(C)OC1CNC(=O)C1]

A solution of 4-hydroxypyrrolidin-2-one (2.00 g, 19.8 mmol, 1.0 eq.), imidazole (2.02 g, 29.7 mmol, 1.5 eq.) and TBSCl (3.58 g, 23.7 mmol, 1.2 eq.) in dichloromethane (20 mL) was stirred for 2 h at room temperature under air atmosphere. The resulting mixture was diluted with water (50 mL) and extracted with EtOAc (2×20 mL). The combined organic layers were concentrated under reduced pressure to yield the title compound as a white solid (3.31 g, 78%).

### 4-[(tert-butyldimethylsilyl)oxy]-1-methylpyrrolidin-2-one

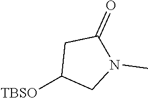

A solution of 4-[(tert-butyldimethylsilyl)oxy]pyrrolidin-2-one (3.00 g, 13.9 mmol, 1.0 eq.) and sodium hydride (400 mg, 16.7 mmol, 1.2 eq.) in DMF (30 mL) was stirred for 1 h at 0° C. under air atmosphere. Then methyl iodide (3.95 g, 27.8 mmol, 2.0 eq.) was added dropwise over 3 minutes at 0° C. The resulting mixture was stirred for 2 h at 0° C. then diluted with water (100 mL) and extracted with ethyl acetate (2×50 mL). The combined organic layers were concentrated under reduced pressure. The residue was purified by flash column chromatography, eluting with ethyl acetate (50%) in 40-60 petroleum ether to afford the title compound as a light yellow solid (2.50 g, 78%).

### 4-hydroxy-1-methylpyrrolidin-2-one

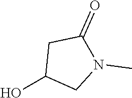

A solution of 4-[(tert-butyldimethylsilyl)oxy]-1-methylpyrrolidin-2-one (1.50 g, 6.54 mmol, 1.0 eq.) and 6M aqueous hydrochloric acid solution (1.99 mL, 65.4 mmol, 10 eq.) in methanol (20 mL) was stirred for 1 h at room temperature under air atmosphere. The mixture was neutralized to pH 7 with saturated aqueous sodium hydrogen carbonate solution.

The resulting mixture was concentrated under reduced pressure then purified by flash column chromatography, eluting with methanol (5%) in dichloromethane to afford the title compound as a colourless oil (586 mg, 78%).

### 1-methyl-4-[2-(methylsulfanyl)-7-oxo-5-[2-(triisopropylsilyl) ethynyl]pyrido[2,3-d]pyrimidin-8-yl]pyrrolidin-2-one

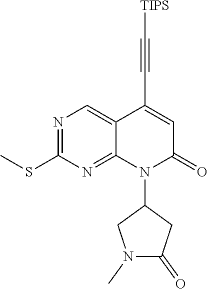

General procedure 18 was applied to 2-(methylsulfanyl)-5-[2-(triisopropylsilyl)ethynyl]-8H-pyrido[2,3-d]pyrimidin-7-one (1.00 g, 0.134 mmol), 4-hydroxy-1-methylpyrrolidin-2-one (230 mg, 0.201 mmol), triphenylphosphine (1.35 g, 0.268 mmol) and DIAD (1.04 g, 0.268 mmol) in THF (20 mL). The residue was purified by flash column chromatography, eluting with methanol (8%) in dichloromethane to afford the title compound as a light yellow oil (318 mg, 43%).

### 4-{2-methanesulfonyl-7-oxo-5-[2-(triisopropylsilyl)ethynyl]pyrido[2,3-d]pyrimidin-8-yl}-1-methylpyrrolidin-2-one

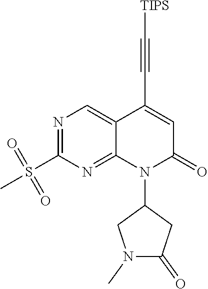

General procedure 10 was applied to 1-methyl-4-[2-(methylsulfanyl)-7-oxo-5-[2-(triisopropylsilyl)ethynyl]pyrido[2,3-d]pyrimidin-8-yl]pyrrolidin-2-one (320 mg, 0.680 mmol) and m-CPBA (117 mg, 0.680 mmol) in dichloromethane (2.0 mL) to yield the title compound as a light yellow oil (303 mg, 89%).

### 1-methyl-4-(2-{[4-(4-methylpiperazin-1-yl)phenyl]amino}-7-oxo-5-[2-(triisopropylsilyl)ethynyl]pyrido[2,3-d]pyrimidin-8-yl)pyrrolidin-2-one

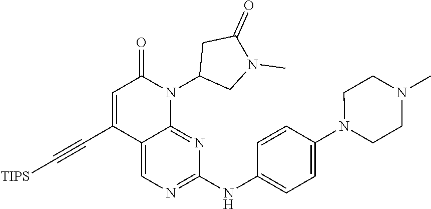

General procedure 13 was applied to 4-{2-methanesulfonyl-7-oxo-5-[2-(triisopropylsilyl)ethynyl]pyrido[2,3-d]pyrimidin-8-yl}-1-methylpyrrolidin-2-one (280 mg, 0.557 mmol), 4-(4-methylpiperazin-1-yl)aniline (160 mg, 0.836 mmol) and trifluoroacetic acid (0.08 mL, 1.114 mmol) in 2-butanol (3.0 mL). The reaction was stirred for 1 h at 100° C. The residue was purified by reverse phase flash column chromatography eluting with acetonitrile (10-60%) in water (0.1% formic acid) to yield the title compound as a red solid (108 mg, 32%).

### 4-(5-ethynyl-2-{[4-(4-methylpiperazin-1-yl)phenyl]amino}-7-oxopyrido[2,3-d]pyrimidin-8-yl)-1-methylpyrrolidin-2-one

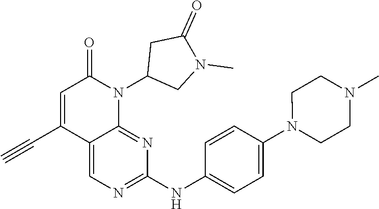 [SMILES: C#Cc1cc(=O)n(C2CC(=O)N(C)C2)c2nc(Nc3ccc(N4CCN(C)CC4)cc3)ncc12]

General procedure 15 was applied to 1-methyl-4-(2-{[4-(4-methylpiperazin-1-yl)phenyl]amino}-7-oxo-5-[2-(triisopropylsilyl)ethynyl]pyrido[2,3-d]pyrimidin-8-yl)pyrrolidin-2-one (170 mg, 0.277 mmol) and potassium fluoride (161 mg, 2.77 mmol) in THF (2.0 mL), DMF (2.0 mL) and water (0.50 mL). The reaction was stirred for 1 h at 60° C. The residue was purified by reverse phase flash column chromatography eluting with acetonitrile (10-60%) in water (0.1% formic acid) to yield the title compound as a red solid (70.0 mg, 55%).

(ES, m/z): [M+H]⁺=458

¹H NMR (400 MHz, DMSO-d₆) δ 9.95 (s, 1H), 9.14 (s, 1H), 7.69 (d, J=7.6 Hz, 2H), 6.94 (d, J=9.4 Hz, 3H), 5.74 (t, J=6.3 Hz, 1H), 5.06 (s, 1H), 3.88 (dd, J=11.7, 5.7 Hz, 1H), 3.49 (d, J=12.1 Hz, 1H), 3.10 (t, J=4.9 Hz, 4H), 2.88 (dd, J=17.7, 7.1 Hz, 1H), 2.76 (s, 3H), 2.47 (t, J=4.9 Hz, 4H), 2.38 (d, J=17.6 Hz, 1H), 2.23 (s, 3H).

### Example 118

### 8-[(1-methylpyrazol-3-yl)methyl]-2-(methylsulfanyl)-5-[2-(triisopropylsilyl)ethynyl]pyrido[2,3-d]pyrimidin-7-one

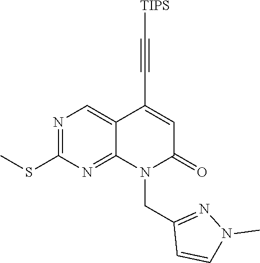

General procedure 18 was applied to 2-(methylsulfanyl)-5-[2-(triisopropylsilyl)ethynyl]-8H-pyrido[2,3-d]pyrimidin-7-one (1.00 g, 2.68 mmol), (1-methylpyrazol-3-yl)methanol (330 mg, 2.95 mmol), triphenylphosphine (1.40 g, 5.35 mmol) and DIAD (1.08 g, 5.35 mmol) in dichloromethane (20 mL). The residue was purified by flash column chromatography, eluting with ethyl acetate (10%) in 40-60 petroleum ether to afford the title compound as a white amorphous solid (1.20 g, 96%).

### 2-methanesulfonyl-8-[(1-methylpyrazol-3-yl)methyl]-5-[2-(triisopropylsilyl)ethynyl]pyrido[2,3-d]pyrimidin-7-one

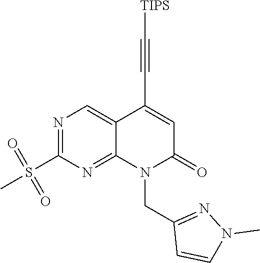

General procedure 10 was applied to 8-[(1-methylpyrazol-3-yl)methyl]-2-(methylsulfanyl)-5-[2-(triisopropylsilyl)ethynyl]pyrido[2,3-d]pyrimidin-7-one (580 mg, 1.24 mmol) and m-CPBA (471 mg, 2.73 mmol) in dichloromethane (10 mL) to yield the title compound as a light yellow oil (578 mg, 93%).

### 2-{[4-(4-methylpiperazin-1-yl)phenyl]amino}-8-[(1-methylpyrazol-3-yl)methyl]-5-[2-(triisopropylsilyl)ethynyl]pyrido[2,3-d]pyrimidin-7-one

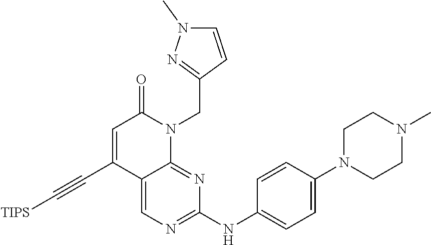

General procedure 13 was applied to 2-methanesulfonyl-8-[(1-methylpyrazol-3-yl)methyl]-5-[2-(triisopropylsilyl)ethynyl]pyrido[2,3-d]pyrimidin-7-one (500 mg, 1.00 mmol), 4-(4-methylpiperazin-1-yl)aniline (229 mg, 1.20 mmol) and trifluoroacetic acid (0.15 mL, 2.00 mmol) in 2-butanol (5 mL). The reaction was stirred overnight at 60° C. The residue was purified by reverse phase flash column chromatography eluting with acetonitrile (10-80%) in water (0.1% formic acid) to yield the title compound as a light yellow solid (160 mg, 26%).

### 5-ethynyl-2-{[4-(4-methylpiperazin-1-yl)phenyl]amino}-8-[(1-methylpyrazol-3-yl)methyl]pyrido[2,3-d]pyrimidin-7-one

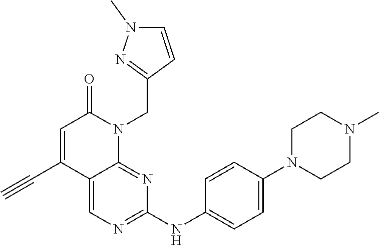 [SMILES: C#Cc1cc(=O)n(Cc2ccn(C)n2)c2nc(Nc3ccc(N4CCN(C)CC4)cc3)ncc12]

General procedure 15 was applied to 2-{[4-(4-methylpiperazin-1-yl)phenyl]amino}-8-[(1-methylpyrazol-3-yl)methyl]-5-[2-(triisopropylsilyl)ethynyl]pyrido[2,3-d]pyrimidin-7-one (160 mg, 0.262 mmol) and potassium fluoride (152 mg, 2.62 mmol) in THF (2.0 mL), DMF (2.0 mL) and water (0.50 mL). The reaction was stirred for 1 h at 60° C. The residue was purified by reverse phase flash column chromatography eluting with acetonitrile (10-50%) in water (0.1% formic acid) to yield the title compound as a yellow solid (63.0 mg, 53%).

(ES, m/z): [M+H]⁺=455

¹H NMR (300 MHz, DMSO-d₆) δ 10.07 (s, 1H), 8.76 (s, 1H), 7.70-7.45 (m, 3H), 6.87 (d, J=9.0 Hz, 2H), 6.57 (s, 1H), 6.05 (d, J=2.2 Hz, 1H), 5.39 (s, 2H), 5.06 (s, 1H), 3.74 (s, 3H), 3.09 (dd, J=6.3, 3.6 Hz, 4H), 2.47 (t, J=5.0 Hz, 4H), 2.24 (s, 3H).

### Example 119

### 3-{[2-(methylsulfanyl)-7-oxo-5-[2-(triisopropylsilyl)ethynyl]pyrido[2,3-d]pyrimidin-8-yl]methyl}pyrrolidin-2-one

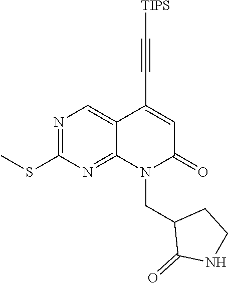

General procedure 18 was applied to 2-(methylsulfanyl)-5-[2-(triisopropylsilyl)ethynyl]-8H-pyrido[2,3-d]pyrimidin-7-one (1.00 g, 2.68 mmol), 3-(hydroxymethyl)pyrrolidin-2-one (340 mg, 2.95 mmol), triphenylphosphine (1.40 g, 5.35 mmol) and DIAD (1.08 g, 5.35 mmol) in THF (10 mL). The residue was purified by reverse phase flash column chromatography eluting with acetonitrile, (65-100%) in water (0.1% trifluoroacetic acid) to yield the title compound as a yellow solid (500 mg, 40%).

### 3-({2-methanesulfonyl-7-oxo-5-[2-(triisopropylsilyl)ethynyl]pyrido[2,3-d]pyrimidin-8-yl}methyl)pyrrolidin-2-one

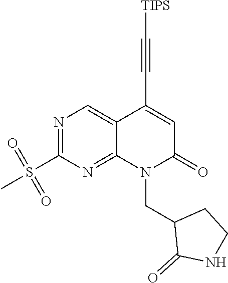

General procedure 10 was applied to 3-{[2-(methylsulfanyl)-7-oxo-5-[2-(triisopropylsilyl)ethynyl]pyrido[2,3-d]pyrimidin-8-yl]methyl}pyrrolidin-2-one (500 mg, 1.06 mmol) and m-CPBA (403 mg, 2.34 mmol) in dichloromethane (5.0 mL) to yield the title compound as a yellow solid (530 mg, 99%).

### 3-[(2-{[4-(4-methylpiperazin-1-yl)phenyl]amino}-7-oxo-5-[2-(triisopropylsilyl)ethynyl]pyrido[2,3-d]pyrimidin-8-yl)methyl]pyrrolidin-2-one

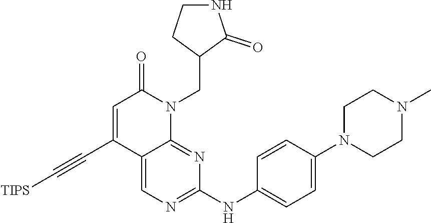

General procedure 13 was applied to 3-({2-methanesulfonyl-7-oxo-5-[2-(triisopropylsilyl)ethynyl]pyrido[2,3-d]pyrimidin-8-yl}methyl)pyrrolidin-2-one (530 mg, 1.05 mmol), trifluoroacetic acid (240 mg, 2.10 mmol) and 4-(4-methylpiperazin-1-yl) aniline (302 mg, 1.58 mmol) in 2-butanol (6.0 mL). The resulting mixture was stirred for 3 h at 100° C. to yield the title compound as an orange solid (330 mg, 51%).

### 3-[(5-ethynyl-2-{[4-(4-methylpiperazin-1-yl)phenyl]amino}-7-oxopyrido[2,3-d]pyrimidin-8-yl)methyl]pyrrolidin-2-one

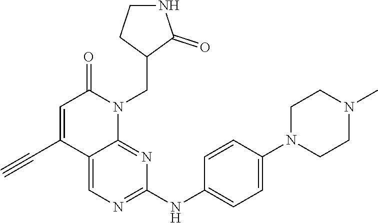 [SMILES: C#Cc1cc(=O)n(CC2CCNC2=O)c2nc(Nc3ccc(N4CCN(C)CC4)cc3)ncc12]

General procedure 15 was applied to 3-[(2-{[4-(4-methylpiperazin-1-yl)phenyl]amino}-7-oxo-5-[2-(triisopropylsilyl)ethynyl]pyrido[2,3-d]pyrimidin-8-yl)methyl]pyrrolidin-2-one (260 mg, 0.424 mmol) and potassium fluoride (246 mg, 4.24 mmol) in THF (3.0 mL), DMF (3.0 mL) and water (260 uL). The reaction was stirred for 2 h at 60° C. The residue was purified by reverse phase flash column chromatography eluting with acetonitrile (10-50%) in water (0.1% trifluoroacetic acid) to yield the title compound as an orange solid (55.8 mg, 28.80%).

(ES, m/z): [M+H]⁺=458.22

¹H-NMR (400 MHz, DMSO-d₆) δ 8.78 (s, 1H), 8.31 (s, 1H), 7.65 (d, J=8.7 Hz, 2H), 7.02-6.90 (m, 2H), 6.54 (s, 1H), 4.90 (d, J=2.0 Hz, 1H), 4.50 (s, 1H), 4.37 (t, J=11.5 Hz, 1H), 3.14 (dt, J=20.4, 3.5 Hz, 6H), 2.96 (p, J=8.4 Hz, 1H), 2.73 (s, 4H), 2.41 (d, J=3.3 Hz, 3H), 2.09-1.77 (m, 2H).

### Example 120

### 2-(methylthio)-5-((triethylsilyl)ethynyl)pyrido[2,3-d]pyrimidin-7(8H)-one

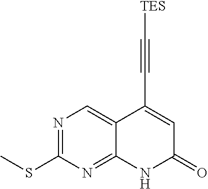

General procedure 9 was applied to 2-(methylthio)-7-oxo-8-phenyl-7,8-dihydropyrido[2,3-d]pyrimidin-5-yl trifluoromethanesulfonate (2.00 g, 4.80 mmol) with triethylsilylacetylene (278 mg, 0.810 mmol), copper(I) iodide (15.4 mg, 81.0 μmol), bis(triphenylphsophine)palladium(II) dichloride (56.8 mg, 81.0 μmol) in DMF (4.0 mL) and N,N-diisopropylethylamine (0.20 mL). The crude material was purified by flash column chromatography eluting with ethyl acetate (25-100%) in 40-60 petroleum ether to yield the title compound as a light yellow solid (180 mg, 0.543 mmol, 67%).

(ES, m/z): [M+H]⁺=332.2

### 2-(methylsulfonyl)-5-((triethylsilyl)ethynyl)pyrido[2,3-d]pyrimidin-7(8H)-one

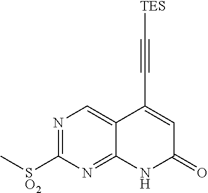

General procedure 10 was applied to 2-(methylthio)-8-phenyl-5-((triethylsilyl)ethynyl)pyrido[2,3-d]pyrimidin-7(8H)-one (170 mg, 0.510 mmol) with m-CPBA (345 mg, 1.54 mmol) in dichloromethane (5.0 mL). The crude material was purified by flash column chromatography eluting with ethyl acetate (25-100%) in 40-60 petroleum ether to yield the title compound as a yellow solid (61.6 mg, 0.169 mmol, 33%).

(ES, m/z): [M+H]⁺=364.2 2-(2-methoxyphenyl)amino-5-((triethylsilyl)ethynyl)-pyrido[2,3-d]pyrimdine-7(8H)-one

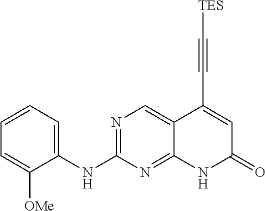

General procedure 13 was applied to 2-(methylsulfonyl)-5-((triethylsilyl)ethynyl)pyrido[2,3-d]pyrimidin-7(8H)-one (61.6 mg, 0.168 mmol) with o-anisidine (30.0 μL, 0.252 mmol) and trifluoroacetic acid (30.0 μL, 0.252 mmol) in acetonitrile (1.7 mL). The crude material was purified by flash column chromatography eluting with methanol (0-2%) in dichloromethane to yield the title compound as a beige solid (28.2 mg, 69.4 μmol, 41%).

(ES, m/z): [M+H]⁺=407.3

### 5-ethynyl-2-((2-methoxyphenyl)amino)pyrido[2,3-d]pyrimidin-7(8H)-one

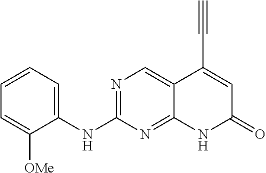 [SMILES: C#Cc1cc(=O)[nH]c2nc(Nc3ccccc3OC)ncc12]

General procedure 16 was applied to 2-(2-methoxyphenyl)amino-5-((triethylsilyl)ethynyl)-pyrido[2,3-d]pyrimdine-7(8H)-one (28.2 mg, 69.4 μmol) with potassium carbonate (10.0 mg, 69.4 μmol) in methanol (1.0 mL). The crude material was purified by flash column chromatography eluting with methanol (0-2%) in dichloromethane to yield the title compound as a beige solid (4.00 mg, 13.7 μmol, 20%).

(ES, m/z): [M+H]⁺=293.1

¹H NMR (500 MHz, CDCl₃) δ 8.85 (s, 1H), 8.29 (d, J=8.6 Hz, 1H), 7.15 (t, J=7.8 Hz, 1H), 7.04 (t, J=7.7 Hz, 1H), 6.97 (d, J=8.1 Hz, 1H), 6.64 (s, 1H), 3.95 (s, 3H), 3.73 (s, 1H).

### Example 121

### Ethyl 4-(methylamino)-2-(methylsulfanyl)pyrimidine-5-carboxylate

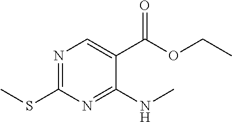

Ethyl 4-chloro-2-(methylsulfanyl)pyrimidine-5-carboxylate (25.0 g, 107 mmol, 1.0 eq.) and methylamine (4.00 g, 129 mmol, 1.2 eq.) in THF (500 mL) stirred for 2 h at room temperature. The reaction was then quenched by the addition of water (500 mL). The resulting solution was extracted with ethyl acetate (2×500 mL). The combined organic layers dried (Na₂SO₄) and concentrated under reduced pressure to yield the title compound as a white solid (20.0 g, 82%).

(ES, m/z): [M+H]⁺=228.1

### 8-methyl-2-(methylsulfanyl)-6-phenyl-6H-pyrido[2,3-d]pyrimidine-5,7-dione

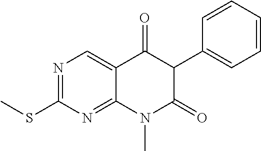 [SMILES: CSc1ncc2c(n1)N(C)C(=O)C(c1ccccc1)C2=O]

General procedure 7 was applied to methyl 4-(methylamino)-2-(methylsulfanyl)pyrimidine-5-carboxylate (5.00 g, 23.4 mmol) with sodium hydride (2.81 g, 117 mmol) and phenylacetyl chloride (4.35 g, 28.1 mmol) in DMF (100 mL) to yield the title compound as a yellow solid (3.00 g, 43%).

(ES, m/z): [M+H]⁺=300.1

### 8-methyl-2-(methylsulfanyl)-7-oxo-6-phenylpyrido[2,3-d]pyrimidin-5-yl trifluoromethanesulfonate

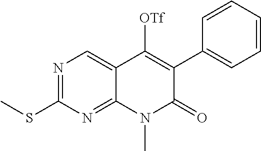 [SMILES: CSc1ncc2c(OS(=O)(=O)C(F)(F)F)c(-c3ccccc3)c(=O)n(C)c2n1]

General procedure 8 was applied to 8-methyl-2-(methylsulfanyl)-6-phenyl-6H-pyrido[2,3-d]pyrimidine-5,7-dione (3.00 g, 10.0 mmol) with triethylamine (2.03 g, 20.0 mmol) and triflic anhydride (4.24 g, 15.0 mmol) in dichloromethane (50 mL). The crude material was purified by flash column chromatography eluting with ethyl acetate (10-15%) in 40-60 petroleum ether to yield the title compound as a light yellow solid (1.60 g, 37%).

(ES, m/z): [M+H]⁺=432.0

### 8-methyl-2-(methylsulfanyl)-6-phenyl-5-[2-(triisopropylsilyl)ethynyl]pyrido[2,3-d]pyrimidin-7-one

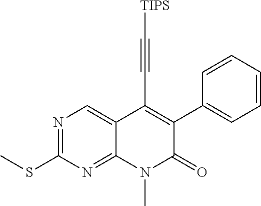

General procedure 9 was applied to 8-methyl-2-(methylsulfanyl)-7-oxo-6-phenylpyrido [2,3-d]pyrimidin-5-yl trifluoromethanesulfonate (1.60 g, 3.71 mmol) with TIPS-acetylene (1.35 g, 7.42 mmol), copper(I) iodide (70.6 mg, 0.371 mmol), bis(triphenylphsophine)palladium (II) chloride (260 mg, 0.371 mmol) and N,N-diisopropylethylamine (1.44 g, 11.1 mmol) in DMF (20 mL). The crude material was purified by flash column chromatography eluting with ethyl acetate (0-100%) petroleum ether to yield the title compound as a light yellow solid (1.40 g, 81%).

(ES, m/z): [M+H]⁺=464.2

### 2-methanesulfonyl-8-methyl-6-phenyl-5-[2-(triisopropylsilyl)ethynyl]pyrido[2,3-d]pyrimidin-7-one

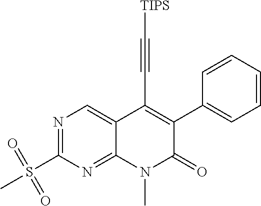

General procedure 10 was applied to 8-methyl-2-(methylsulfanyl)-6-phenyl-5-[2-(triisopropylsilyl)ethynyl]pyrido[2,3-d]pyrimidin-7-one (1.40 g, 3.02 mmol) with m-CPBA (1.56 g, 9.04 mmol) in dichloromethane (20 mL). The crude material was purified by flash column chromatography eluting with ethyl acetate (5%) in 40-60 petroleum ether to yield the title compound as a light yellow solid (1.20 g, 80%).

(ES, m/z): [M+H]⁺=496.2

### 2-[(4-[[2-(dimethylamino)ethyl](methyl)amino]-2-methoxyphenyl)amino]-8-methyl-6-phenyl-5-[2-(triisopropylsilyl)ethynyl]pyrido[2,3-d]pyrimidin-7-one

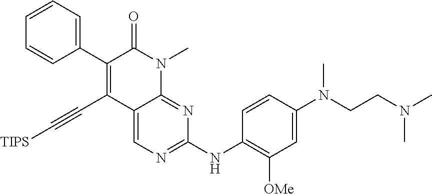

General procedure 13 was applied to 2-methanesulfonyl-8-methyl-6-phenyl-5-[2-(triisopropylsilyl)ethynyl]pyrido[2,3-d]pyrimidin-7-one (500 mg, 1.01 mmol) with N¹-[2-(dimethylamino)ethyl]-3-methoxy-N¹-methylbenzene-1,4-diamine (270 mg, 1.21 mmol) and trifluoroacetic acid (138 mg, 1.21 mmol) in 2-butanol (5 mL). The crude product was purified by preparative-HPLC eluting with methanol (22-65%) in water (0.05% trifluoroacetic acid) to yield the title compound as a red solid (550 mg, 85%).

(ES, m/z): [M+H]⁺=639.4

### 2-[(4-[[2-(dimethylamino)ethyl](methyl)amino]-2-methoxyphenyl)amino]-5-ethynyl-8-methyl-6-phenylpyrido[2,3-d]pyrimidin-7-one

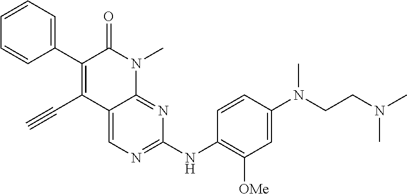 [SMILES: C#Cc1c(-c2ccccc2)c(=O)n(C)c2nc(Nc3ccc(N(C)CCN(C)C)cc3OC)ncc12]

A solution of 2-[(4-[[2-(dimethylamino)ethyl](methyl)amino]-2-methoxyphenyl)amino]-8-methyl-6-phenyl-5-[2-(triisopropylsilyl)ethynyl]pyrido[2,3-d]pyrimidin-7-one (100 mg, 0.157 mmol, 1.0 eq.), TBAF (32.7 mg, 0.126 mmol, 0.80 eq.) in THF (3.0 mL) was stirred for 20 min at room temperature. The reaction was quenched with water (4.0 mL) and extracted with ethyl acetate (2×5 mL). The combined organic layers were concentrated under reduced pressure. The crude product was purified by Preparative-HPLC eluting with acetonitrile (30-70%) in water (0.05% formic acid) to yield the title compound as a dark brown solid (20.0 mg, 26%).

ES, m/z): [M+H]⁺=483.2

¹H NMR (300 MHz, CDCl₃) δ 8.94 (s, 1H), 8.29 (d, J=8.8 Hz, 1H), 8.08 (s, 1H), 7.59-7.50 (m, 2H), 7.50-7.36 (m, 3H), 6.52 (d, J=2.6 Hz, 1H), 6.49-6.36 (m, 1H), 3.97 (s, 3H), 3.78 (s, 5H), 3.57 (s, 1H), 3.21 (t, J=7.0 Hz, 2H), 3.01 (s, 3H), 2.89 (s, 6H).

### Example 122

### 6-(2-chlorophenyl)-8-methyl-2-(methylsulfanyl)-6H-pyrido [2,3-d]pyrimidine-5,7-dione

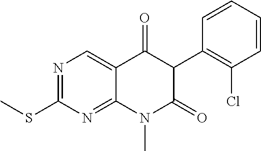 [SMILES: CSc1ncc2c(n1)N(C)C(=O)C(c1ccccc1Cl)C2=O]

General procedure 7 was applied to ethyl 4-(methylamino)-2-(methylsulfanyl)pyrimidine-5-carboxylate (5.00 g, 22.0 mmol) with sodium hydride (2.64 g, 110 mmol), (2-chlorophenyl)acetyl chloride (8.32 g, 44.0 mmol) in DMF (50 mL) to yield the title compound as a yellow solid (3.00 g, 41%).

(ES, m/z): [M+H]⁺=334.0

### 6-(2-chlorophenyl)-8-methyl-2-(methylsulfanyl)-7-oxopyrido [2,3-d]pyrimidin-5-yl trifluoromethanesulfonate

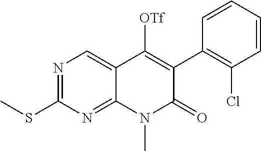 [SMILES: CSc1ncc2c(OS(=O)(=O)C(F)(F)F)c(-c3ccccc3Cl)c(=O)n(C)c2n1]

General procedure 8 was applied to 6-(2-chlorophenyl)-8-methyl-2-(methylsulfanyl)-6H-pyrido[2,3-d]pyrimidine-5,7-dione (1.50 g, 4.49 mmol) with triethylamine (1.36 g, 13.5 mmol) and triflic anhydride (1.90 g, 6.70 mmol) in dichloromethane (50 mL). The crude material was purified by flash column chromatography eluting with ethyl acetate (15%) in 40-60 petroleum ether to yield the title compound as a light yellow solid (1.50 g, 72%).

(ES, m/z): [M+H]⁺=466.0 6-(2-chlorophenyl)-8-methyl-2-(methylsulfanyl)-5-[2-(triisopropylsilyl)ethynyl]pyrido[2,3-d]pyrimidin-7-one

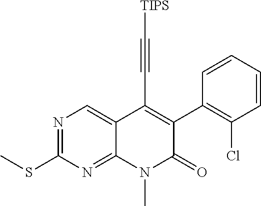

General procedure 9 was applied to 6-(2-chlorophenyl)-8-methyl-2-(methylsulfanyl)-7-oxopyrido[2,3-d]pyrimidin-5-yl trifluoromethanesulfonate (1.50 g, 3.22 mmol) with triisopropylsilylacetylene (1.17 g, 6.44 mmol), copper(I) iodide (61.3 mg, 0.322 mmol), bis(triphenylphosphine)palladium(II) dichloride (226 mg, 0.322 mmol) and N,N-diisopropylethylamine (1.25 g, 9.66 mmol) in DMF (30 mL). The crude material was purified by flash column chromatography eluting with ethyl acetate (0-100%) in 40-60 petroleum ether to yield the title compound as a light yellow solid (1.20 g, 75%).

(ES, m/z): [M+H]⁺=498.2

### 6-(2-chlorophenyl)-2-methanesulfonyl-8-methyl-5-[2-(triisopropylsilyl)ethynyl]pyrido[2,3-d]pyrimidin-7-one

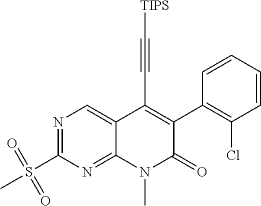

General procedure 10 was applied to 6-(2-chlorophenyl)-8-methyl-2-(methylsulfanyl)-5-[2-(triisopropylsilyl)ethynyl]pyrido[2,3-d]pyrimidin-7-one (1.20 g, 2.41 mmol) with m-CPBA (1.25 g, 7.23 mmol) in dichloromethane (10 mL). The crude material was purified by flash column chromatography eluting with ethyl acetate (5%) in 40-60 petroleum ether to yield the title compound as a light yellow solid (1.00 g, 78%).

(ES, m/z): [M+H]⁺=530.2

### 6-(2-chlorophenyl)-2-[(4-{[2-(dimethylamino)ethyl](methyl)amino}-2-methoxyphenyl)amino]-8-methyl-5-[2-(triisopropylsilyl)ethynyl]pyrido[2,3-d]pyrimidin-7-one

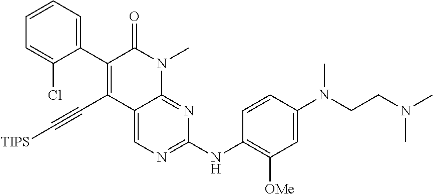

General procedure 13 was applied to 6-(2-chlorophenyl)-2-methanesulfonyl-8-methyl-5-[2-(triisopropylsilyl)ethynyl]pyrido[2,3-d]pyrimidin-7-one (300 mg, 0.566 mmol) with N¹-[2-(dimethylamino)ethyl]-3-methoxy-N¹-methylbenzene-1,4-diamine (189 mg, 0.849 mmol) and trifluoroacetic acid (96.8 mg, 0.849 mmol) in 2-butanol (10 mL). The crude product was purified by Prep-HPLC eluting with methanol (22-65%) in water (0.05% trifluoroacetic acid) to yield the title compound as a red solid (100 mg, 26%).

(ES, m/z): [M+H]⁺=673.3

### 6-(2-chlorophenyl)-2-[(4-[[2-(dimethylamino)ethyl](methyl)amino]-2-methoxyphenyl)amino]-5-ethynyl-8-methylpyrido[2,3-d]pyrimidin-7-one

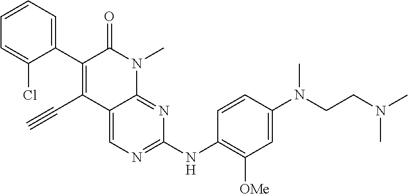 [SMILES: C#Cc1c(-c2ccccc2Cl)c(=O)n(C)c2nc(Nc3ccc(N(C)CCN(C)C)cc3OC)ncc12]

A solution of 6-(2-chlorophenyl)-2-[(4-[[2-(dimethylamino)ethyl](methyl)amino]-2-methoxyphenyl)amino]-8-methyl-5-[2-(triisopropylsilyl)ethynyl]pyrido[2,3-d]pyrimidin-7-one (100 mg, 0.149 mmol, 1.0 eq.) and TBAF (31.1 mg, 0.119 mmol) in THF (3.0 mL) was stirred for 20 min at room temperature. The reaction was then quenched with water (4.0 mL) and extracted with ethyl acetate (2×5 mL). The combined organic layers were concentrated under reduced pressure. The crude product was purified by Prep-HPLC eluting with acetonitrile (30-70%) in water (0.05% formic acid) to yield the title compound as a red solid (20.0 mg, 26%).

(ES, m/z): [M+H]⁺=517.2

¹H NMR (300 MHz, CDCl₃) δ 8.94 (s, 1H), 8.34 (d, J=8.8 Hz, 1H), 8.01 (s, 1H), 7.58-7.46 (m, 1H), 7.38 (tdd, J=6.9, 4.7, 2.9 Hz, 3H), 6.54 (d, J=2.6 Hz, 1H), 6.45 (dd, J=8.9, 2.6 Hz, 1H), 3.98 (s, 3H), 3.81 (d, J=7.0 Hz, 5H), 3.55 (s, 1H), 3.23 (t, J=6.9 Hz, 2H), 3.01 (s, 3H), 2.91 (s, 6H)

### Example 123

### 6-(2-chlorophenyl)-2-[(2-methoxyphenyl)amino]-8-methyl-5-[2-(triisopropylsilyl)ethynyl]pyrido[2,3-d]pyrimidin-7-one

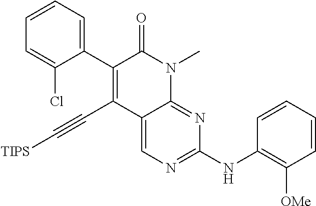

General procedure 13 was applied to 6-(2-chlorophenyl)-2-methanesulfonyl-8-methyl-5-[2-(triisopropylsilyl)ethynyl]pyrido[2,3-d]pyrimidin-7-one (300 mg, 0.566 mmol) with o-anisidine (105 mg, 0.849 mmol) and trifluoroacetic acid (104 mg, 0.907 mmol) in 2-butanol (10 mL). Stirred overnight at 100° C. The crude product was purified by Prep-HPLC to yield the title compound as a light yellow solid (100 mg, 31%).

(ES, m/z): [M+H]⁺=573.2

### 6-(2-chlorophenyl)-5-ethynyl-2-[(2-methoxyphenyl)amino]-8-methylpyrido[2,3-d]pyrimidin-7-one

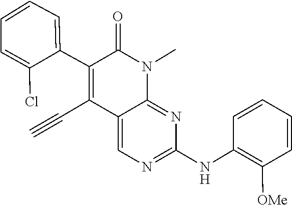 [SMILES: C#Cc1c(-c2ccccc2Cl)c(=O)n(C)c2nc(Nc3ccccc3OC)ncc12]

General procedure 15 was applied to 6-(2-chlorophenyl)-2-[(2-methoxyphenyl)amino]-8-methyl-5-[2-(triisopropylsilyl)ethynyl]pyrido[2,3-d]pyrimidin-7-one (100 mg, 0.174 mmol) with potassium fluoride (101 mg, 1.74 mmol) in THF (2.0 mL) and water (0.50 mL). The resulting solution was stirred overnight at 50° C. The crude product was purified by Prep-HPLC eluting with acetonitrile (30-70%) in water (0.05% formic acid) to yield the title compound as a yellow solid (20 mg, 27.5%).

(ES, m/z): [M+H]⁺=417.1

¹H NMR (300 MHz, DMSO-d₆) δ 8.95 (s, 1H), 8.86 (s, 1H), 8.01 (d, J=7.8 Hz, 1H), 7.63-7.53 (m, 1H), 7.53-7.31 (m, 3H), 7.26-7.09 (m, 2H), 7.08-6.92 (m, 1H), 4.91 (s, 1H), 3.87 (s, 3H), 3.58 (s, 3H).

### Example 124

### 6-(2-chlorophenyl)-8-methyl-2-(phenylamino)-5-[2-(triisopropylsilyl)ethynyl]pyrido[2,3-d]pyrimidin-7-one

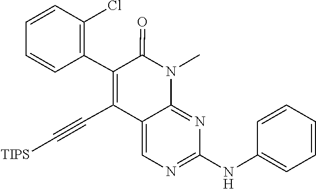

General procedure 13 was applied to 6-(2-chlorophenyl)-2-methanesulfonyl-8-methyl-5-[2-(triisopropylsilyl)ethynyl]pyrido[2,3-d]pyrimidin-7-one (120 mg, 0.226 mmol), aniline (21.1 mg, 0.226 mmol) and trifluoroacetic acid (25.8 mg, 0.226 mmol) in 2-butanol (10 mL). The resulting mixture was stirred for 1 h at 110° C. The residue was purified by reverse phase flash chromatography eluting with acetonitrile (20-100%) in water to yield the title compound as a yellow solid (100 mg, 81.34%).

(ES, m/z): [M+H]⁺=544.2

### 6-(2-chlorophenyl)-5-ethynyl-8-methyl-2-(phenylamino)pyrido[2,3-d]pyrimidin-7-one

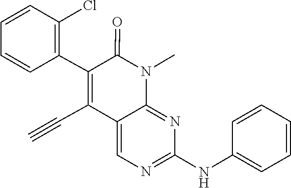 [SMILES: C#Cc1c(-c2ccccc2Cl)c(=O)n(C)c2nc(Nc3ccccc3)ncc12]

General procedure 15 was applied to 6-(2-chlorophenyl)-8-methyl-2-(phenylamino)-5-[2-(triisopropylsilyl)ethynyl]pyrido[2,3-d]pyrimidin-7-one (100 mg, 0.184 mmol, 1.00 eq.) with potassium fluoride (107 mg, 1.84 mmol) in methanol (1.0 mL). The resulting mixture was stirred for 2 h at 70° C. The residue was purified by reverse phase flash column chromatography eluting with methanol (20-100%) in water to yield the title compound as a yellow solid (25.0 mg, 35%).

(ES, m/z): [M+H]⁺=387.1

¹H NMR (300 MHz, DMSO-d₆) δ 10.32 (s, 1H), 8.91 (s, 1H), 7.91-7.80 (m, 2H), 7.65-7.52 (m, 1H), 7.51-7.32 (m, 5H), 7.11-7.00 (m, 1H), 4.92 (s, 1H), 3.66 (s, 3H).

### Example 125

### 6-(2,6-dichlorophenyl)-8-methyl-2-(methylsulfanyl)-6H-pyrido[2,3-d]pyrimidine-5,7-dione

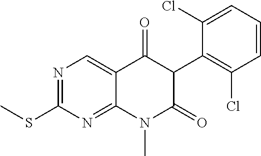 [SMILES: CSc1ncc2c(n1)N(C)C(=O)C(c1c(Cl)cccc1Cl)C2=O]

General procedure 7 was applied to ethyl 4-(methylamino)-2-(methylsulfanyl)pyrimidine-5-carboxylate (5.00 g, 22.0 mmol), sodium hydride (2.64 g, 110 mmol) and (2,6-dichlorophenyl)acetyl chloride (9.83 g, 44.0 mmol) in DMF (50 mL). The reaction was then quenched by the addition of ice-water (200 mL). The resulting solution was extracted with ethyl acetate (2×200 mL) and the aqueous layers combined. The pH value of the solution was adjusted to 5 with 5% aqueous hydrochloric acid. The solids were collected by filtration to yield the title compound as an off-white solid (3.00 g, 37%).

(ES, m/z): [M+H]⁺=368.0

### 6-(2,6-dichlorophenyl)-8-methyl-2-(methylsulfanyl)-7-oxopyrido[2,3-d]pyrimidin-5-yl trifluoromethanesulfonate

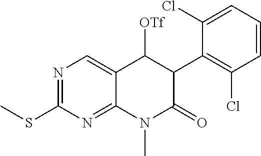 [SMILES: CSc1ncc2c(n1)N(C)C(=O)C(c1c(Cl)cccc1Cl)C2OS(=O)(=O)C(F)(F)F]

General procedure 8 was applied to 6-(2,6-dichlorophenyl)-8-methyl-2-(methylsulfanyl)-6H-pyrido[2,3-d]pyrimidine-5,7-dione (3.00 g, 8.15 mmol), triethylamine (2.47 g, 24.4 mmol) and triflic anhydride (3.45 g, 12.2 mmol) in dichloromethane (50 mL). The residue was purified by flash column chromatography eluting with ethyl acetate (10%) in 40-60 petroleum ether to yield the title compound as a yellow solid (3.00 g, 73%).

(ES, m/z): [M+H]⁺=499.9

### 6-(2,6-dichlorophenyl)-8-methyl-2-(methylsulfanyl)-5-[2-(triisopropylsilyl)ethynyl]pyrido[2,3-d]pyrimidin-7-one

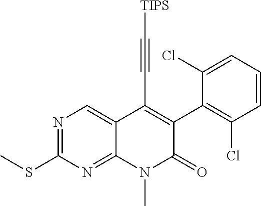

General procedure 9 was applied to 6-(2,6-dichlorophenyl)-8-methyl-2-(methylsulfanyl)-7-oxopyrido[2,3-d]pyrimidin-5-yl trifluoromethanesulfonate (1.50 g, 2.99 mmol), triisopropylsilylacetylene (1.09 g, 5.99 mmol), copper(I) iodide (57.1 mg, 0.300 mmol), bis(triphenylphsophine)palladium(II) dichloride (210 mg, 0.300 mmol) and N,N-diisopropylethylamine (1.16 g, 8.99 mmol) in DMF (20 mL). The residue was purified by flash column chromatography eluting with 40-60 petroleum ether to yield the title compound as a yellow solid (1.00 g, 63%).

(ES, m/z): [M+H]⁺=532.1

### 6-(2,6-dichlorophenyl)-2-methanesulfonyl-8-methyl-5-[2-(triisopropylsilyl)ethynyl]pyrido[2,3-d]pyrimidin-7-one

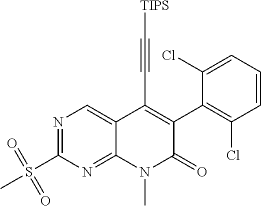

General procedure 10 was applied to 6-(2,6-dichlorophenyl)-8-methyl-2-(methylsulfanyl)-5-[2-(triisopropylsilyl)ethynyl]pyrido[2,3-d]pyrimidin-7-one (1.00 g, 1.88 mmol) and m-CPBA (970 mg, 5.63 mmol) in dichloromethane (10 mL). The residue was purified by flash column chromatography eluting with ethyl acetate (4%) in 40-60 petroleum ether to yield the title compound as a yellow solid (900 mg, 85%).

(ES, m/z): [M+H]⁺=564.1

### 6-(2,6-dichlorophenyl)-2-{[3-(hydroxymethyl)phenyl]amino}-8-methyl-5-[2-(triisopropylsilyl)ethynyl]pyrido[2,3-d]pyrimidin-7-one

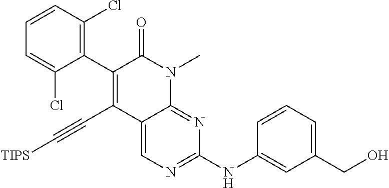

General procedure 13 was applied to 6-(2,6-dichlorophenyl)-2-methanesulfonyl-8-methyl-5-[2-(triisopropylsilyl)ethynyl]pyrido[2,3-d]pyrimidin-7-one (400 mg, 0.708 mmol) with (3-aminophenyl)methanol (131 mg, 1.06 mmol) and trifluoroacetic acid (121 mg, 1.06 mmol) in 2-butanol (10 mL). The reaction was stirred overnight at 100° C. The residue was purified by reverse phase flash column chromatography eluting with acetonitrile (50-100%) in water to yield the title compound as a light yellow solid (100 mg, 23%).

(ES, m/z): [M+H]⁺=573.2

### 6-(2,6-dichlorophenyl)-5-ethynyl-2-{[3-(hydroxymethyl)phenyl]amino}-8-methylpyrido[2,3-d]pyrimidin-7-one

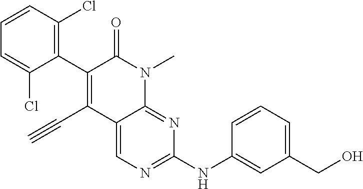 [SMILES: C#Cc1c(-c2c(Cl)cccc2Cl)c(=O)n(C)c2nc(Nc3cccc(CO)c3)ncc12]

General procedure 15 was applied to 6-(2,6-dichlorophenyl)-2-{[3-(hydroxymethyl)phenyl]amino}-8-methyl-5-[2-(triisopropylsilyl)ethynyl]pyrido[2,3-d]pyrimidin-7-one (100 mg, 0.165 mmol) with potassium fluoride (95.6 mg, 1.65 mmol) in THF (2.0 mL) and water (0.50 mL). The resulting solution was stirred overnight at 50° C. The crude product was purified by Prep-HPLC eluting with acetonitrile (50-68%) in water (0.1% NH₄HCO₃) to yield the title compound as a light yellow solid (20.0 mg, 27%).

(ES, m/z): [M+H]⁺=451.1

¹H NMR (400 MHz, DMSO-d₆) δ 10.38 (s, 1H), 8.92 (s, 1H), 7.90 (s, 1H), 7.72-7.64 (m, 1H), 7.64-7.57 (m, 2H), 7.49 (dd, J=8.8, 7.3 Hz, 1H), 7.31 (t, J=7.8 Hz, 1H), 7.02 (d, J=7.6 Hz, 1H), 5.21 (t, J=5.7 Hz, 1H), 4.97 (s, 1H), 4.52 (d, J=5.7 Hz, 2H), 3.67 (s, 3H).

### Example 126

### 6-(2-chlorophenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one

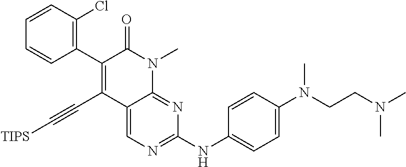

General procedure 13 was applied to 6-(2-chlorophenyl)-2-methanesulfonyl-8-methyl-5-[2-(triisopropylsilyl)ethynyl]pyrido[2,3-d]pyrimidin-7-one (120 mg, 0.226 mmol) with N¹-[2-(dimethylamino)ethyl]-N¹-methylbenzene-1,4-diamine (43.7 mg, 0.226 mmol) and trifluoroacetic acid (25.81 mg, 0.226 mmol) in 2-butanol (20 mL). The resulting mixture was stirred for 1 h at 100° C. The residue was purified by reverse phase flash column chromatography eluting with acetonitrile (20-100%) in water to yield the title compound as a red oil (80 mg, 55%).

(ES, m/z): [M+H]⁺=644.3

### 6-(2-chlorophenyl)-5-ethynyl-8-methyl-2-[(4-{methyl[2(methylamino)ethyl]amino}phenyl)amino]pyrido[2,3-d]pyrimidin-7-one

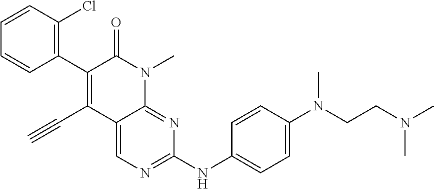 [SMILES: C#Cc1c(-c2ccccc2Cl)c(=O)n(C)c2nc(Nc3ccc(N(C)CCN(C)C)cc3)ncc12]

General procedure 15 was applied to 6-(2-chlorophenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one (80.0 mg, 0.124 mmol) with potassium fluoride (7.22 mg, 0.124 mmol) in methanol (2.0 mL). The resulting mixture was stirred for 30 min at 70° C. The residue was purified by reverse phase flash column chromatography eluting with acetonitrile (30-100%) in water to yield the title compound as a light red solid (10.0 mg, 17%).

(ES, m/z): [M+H]⁺=487.3

¹H NMR (300 MHz, DMSO-d6) δ 10.04 (s, 1H), 8.82 (s, 1H), 7.74-7.54 (m, 3H), 7.44-7.34 (m, 3H), 6.72 (d, J=8.8 Hz, 2H), 4.88 (s, 1H), 3.51 (s, 2H), 2.90 (s, 3H), 2.38 (t, J=7.2 Hz, 2H), 2.19 (s, 6H).

### Example 127

### 6-(2-chlorophenyl)-2-{[2-methoxy-4-(4-methylpiperazin-1-yl)phenyl]amino}-8-methyl-5-[2-(triisopropylsilyl)ethynyl]pyrido[2,3-d]pyrimidin-7-one

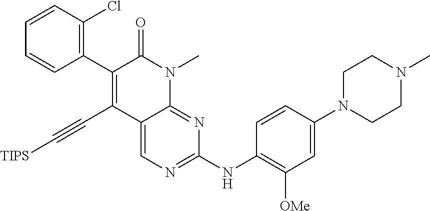

General procedure 13 was applied to 6-(2-chlorophenyl)-2-methanesulfonyl-8-methyl-5-[2-(triisopropylsilyl)ethynyl]pyrido[2,3-d]pyrimidin-7-one (110 mg, 0.207 mmol) and 2-methoxy-4-(4-methylpiperazin-1-yl)aniline (45.9 mg, 0.207 mmol) and trifluoroacetic acid (23.6 mg, 0.207 mmol) in 2-butanol (2.0 mL). The resulting mixture was stirred for 1 h at 100° C. . The residue was purified by reverse phase flash column chromatography eluting with acetonitrile (20-100%) in water to yield the title compound as a reddish solid (100 mg, 72%).

(ES, m/z): [M+H]⁺=671.3

### 6-(2-chlorophenyl)-5-ethynyl-2-{[2-methoxy-4-(4-methylpiperazin-1-yl)phenyl]amino}-8-methylpyrido[2,3-d]pyrimidin-7-one

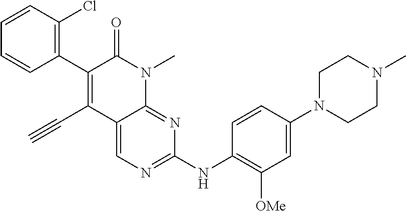 [SMILES: C#Cc1c(-c2ccccc2Cl)c(=O)n(C)c2nc(Nc3ccc(N4CCN(C)CC4)cc3OC)ncc12]

General procedure 15 was applied to 6-(2-chlorophenyl)-2-{[2-methoxy-4-(4-methylpiperazin-1-yl)phenyl]amino}-8-methyl-5-[2-(triisopropylsilyl)ethynyl]pyrido[2,3-d]pyrimidin-7-one (100 mg, 0.149 mmol) and potassium fluoride (86.5 mg, 1.49 mmol) in methanol. The resulting mixture was stirred for 1 h at 70° C. The residue was purified by reverse phase flash column chromatography eluting with acetonitrile (20-80%) in water, to yield the title compound as a reddish solid (30.0 mg, 39%).

(ES, m/z): [M+H]⁺=515.3

¹H NMR (300 MHz, DMSO-d6) δ 8.82 (d, J=16.9 Hz, 2H), 7.65 (s, 1H), 7.55 (dt, J=6.9, 2.9 Hz, 1H), 6.66 (d, J=2.5 Hz, 1H), 6.54 (dd, J=8.8, 2.5 Hz, 1H), 4.86 (s, 1H), 3.82 (s, 3H), 3.54 (s, 3H), 3.17 (dd, J=6.4, 3.6 Hz, 4H), 2.47 (t, J=5.0 Hz, 4H), 2.24 (s, 3H).

### Example 128

### 6-(2-chlorophenyl)-8-methyl-2-{[4-(4-methylpiperazin-1-yl)phenyl]amino}-5-[2-(triisopropylsilyl)ethynyl]pyrido[2,3-d]pyrimidin-7-one

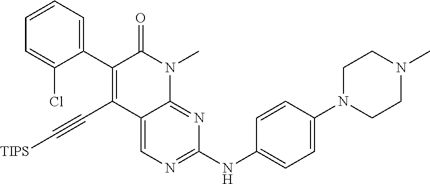

General procedure 13 was applied to 6-(2-chlorophenyl)-2-methanesulfonyl-8-methyl-5-[2-(triisopropylsilyl)ethynyl]pyrido[2,3-d]pyrimidin-7-one (300 mg, 0.566 mmol) with 4-(4-methylpiperazin-1-yl)aniline (162 mg, 0.849 mmol) and trifluoroacetic acid (96.8 mg, 0.849 mmol) in 2-butanol (10 mL). The reaction mixture was stirred overnight at 100° C. The crude product was purified by Prep-HPLC eluting with acetonitrile (40-68%) in water (0.1% NH₄HCO₃) to yield the title compound as a light yellow solid in (100 mg, 27.5%).

(ES, m/z): [M+H]⁺=641.3

### 6-(2-chlorophenyl)-5-ethynyl-8-methyl-2-{[4-(4-methylpiperazin-1-yl)phenyl]amino}pyrido[2,3-d]pyrimidin-7-one

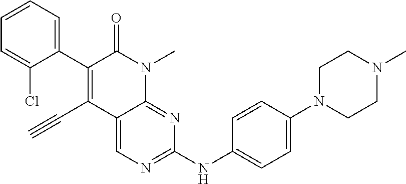 [SMILES: C#Cc1c(-c2ccccc2Cl)c(=O)n(C)c2nc(Nc3ccc(N4CCN(C)CC4)cc3)ncc12]

General procedure 15 was applied to 6-(2-chlorophenyl)-8-methyl-2-{[4-(4-methylpiperazin-1-yl)phenyl]amino}-5-[2-(triisopropylsilyl)ethynyl]pyrido[2,3-d]pyrimidin-7-one (100 mg, 0.156 mmol) with potassium fluoride (90.6 mg, 1.56 mmol) in THF (3.0 mL) and water (0.50 mL). The resulting mixture was stirred overnight at room temperature. The crude product was purified by Prep-HPLC eluting with (30-70%) in water (0.05% formic acid) to yield the title compound as a yellow solid (20.0 mg, 26%).

(ES, m/z): [M+H]⁺=485.2

¹H NMR (400 MHz, DMSO-d6) δ 10.12 (s, 1H), 8.84 (s, 1H), 7.67 (d, J=8.2 Hz, 2H), 7.60-7.51 (m, 1H), 7.48-7.38 (m, 2H), 7.39-7.32 (m, 1H), 6.95 (d, J=9.0 Hz, 2H), 4.88 (s, 1H), 3.62 (s, 3H), 3.10 (t, J=5.0 Hz, 4H), 2.45 (t, J=5.0 Hz, 4H), 2.22 (s, 3H).

### Example 129

### 6-(2-chlorophenyl)-2-({4-[2-(dimethylamino)ethoxy]phenyl}amino)-8-methyl-5-[2-(triisopropylsilyl)ethynyl]pyrido[2,3-d]pyrimidin-7-one

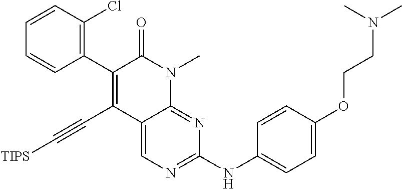

General procedure 13 was applied to 6-(2-chlorophenyl)-2-methanesulfonyl-8-methyl-5-[2-(triisopropylsilyl)ethynyl]pyrido[2,3-d]pyrimidin-7-one (120 mg, 0.226 mmol) with 4-[2-(dimethylamino)ethoxy]aniline (40.8 mg, 0.226 mmol) and trifluoroacetic acid (25.8 mg, 0.226 mmol) in 2-butanol (2.0 mL). The resulting mixture was stirred for 1 h at 100° C. The residue was purified by reverse phase flash column chromatography eluting with acetonitrile (20-80%) in water to yield the title compound as a yellow oil (120 mg, 84%).

(ES, m/z): [M+H]⁺=630.3

### 6-(2-chlorophenyl)-2-({4-[2-(dimethylamino)ethoxy]phenyl}amino)-5-ethynyl-8-methylpyrido[2,3-d]pyrimidin-7-one

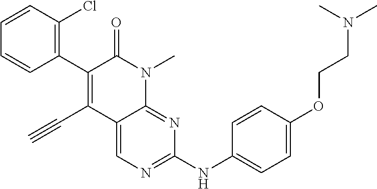 [SMILES: C#Cc1c(-c2ccccc2Cl)c(=O)n(C)c2nc(Nc3ccc(OCCN(C)C)cc3)ncc12]

General procedure 15 was applied to 6-(2-chlorophenyl)-2-({4-[2-(dimethylamino)ethoxy]phenyl}amino)-8-methyl-5-[2-(triisopropylsilyl)ethynyl]pyrido[2,3-d]pyrimidin-7-one (90.0 mg, 0.143 mmol) and potassium fluoride (82.9 mg, 1.43 mmol) in methanol (5 mL). The resulting mixture was stirred for 2 h at 80° C. The residue was purified by reverse phase flash column chromatography eluting with methanol (20-80%) in water to yield the title compound as a yellow solid (35.0 mg, 52%).

(ES, m/z): [M+H]⁺=474.3

¹H NMR (300 MHz, DMSO-d6) δ 10.18 (s, 1H), 8.86 (s, 1H), 7.72 (d, J=8.5 Hz, 2H), 7.60-7.50 (m, 1H), 7.48-7.35 (m, 3H), 7.01-6.90 (m, 2H), 4.90 (s, 1H), 4.04 (t, J=5.9 Hz, 2H), 3.63 (s, 3H), 2.62 (t, J=5.9 Hz, 2H), 2.55 (s, 1H), 2.22 (s, 6H).

### Example 130

### 6-(2-chlorophenyl)-8-methyl-2-{[4-(4-methylpiperazin-1-yl)butyl]amino}-5-[2-(triisopropylsilyl)ethynyl]pyrido[2,3-d]pyrimidin-7-one

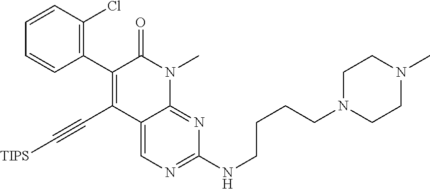

A solution of 6-(2-chlorophenyl)-2-methanesulfonyl-8-methyl-5-[2-(triisopropylsilyl)ethynyl]pyrido[2,3-d]pyrimidin-7-one (120 mg, 0.226 mmol, 1.0 eq.), 4-(4-methylpiperazin-1-yl)butan-1-amine (38.8 mg, 0.226 mmol, 1.0 eq.) and NMP (2.0 mL, 20.7 mmol, 92 eq.) at room temperature. The resulting mixture was stirred for 1 h at 100° C. The residue was purified by reverse phase flash column chromatography eluting with acetonitrile (20-80%) in water to yield the title compound as a yellow oil (130 mg, 92%).

(ES, m/z): [M+H]⁺=621.3

### 6-(2-chlorophenyl)-5-ethynyl-8-methyl-2-{[4-(4-methylpiperazin-1-yl)butyl]amino}pyrido[2,3-d]pyrimidin-7-one

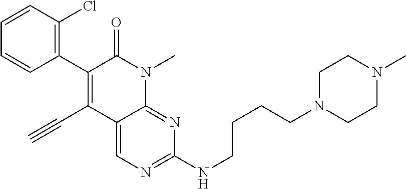 [SMILES: C#Cc1c(-c2ccccc2Cl)c(=O)n(C)c2nc(NCCCCN3CCN(C)CC3)ncc12]

General procedure 15 was applied to 6-(2-chlorophenyl)-8-methyl-2-{[4-(4-methylpiperazin-1-yl)butyl]amino}-5-[2-(triisopropylsilyl)ethynyl]pyrido[2,3-d]pyrimidin-7-one (120 mg, 0.193 mmol) and potassium fluoride (112 mg, 1.93 mmol) in methanol (5 mL). The resulting mixture was stirred for 2 h at 70° C. The residue was purified by reverse phase flash column chromatography eluting with acetonitrile (18-70%) in water, to yield the title compound as an off-white solid (80.0 mg, 91%).

(ES, m/z): [M+H]⁺=465.2

¹H NMR (300 MHz, DMSO-d6) δ 8.74 (d, J=20.2 Hz, 1H), 8.24-7.97 (m, 1H), 7.62-7.50 (m, 1H), 7.46-7.29 (m, 3H), 4.83 (s, 1H), 3.58 (s, 2H), 3.54 (s, 1H), 3.40 (d, J=6.4 Hz, 2H), 2.50-2.20 (m, 10H), 2.13 (s, 3H), 1.56 (dq, J=31.9, 7.3 Hz, 4H).

### Example 131

### 6-(2-chlorophenyl)-2-[(3-methoxyphenyl)amino]-8-methyl-5-[2-(triisopropylsilyl)ethynyl]pyrido[2,3-d]pyrimidin-7-one

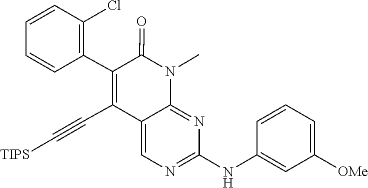

General procedure 13 was applied to 6-(2-chlorophenyl)-2-methanesulfonyl-8-methyl-5-[2-(triisopropylsilyl)ethynyl]pyrido[2,3-d]pyrimidin-7-one (110 mg, 0.207 mmol) with m-anisidine (25.6 mg, 0.207 mmol) and trifluoroacetic acid (23.7 mg, 0.207 mmol) in 2-butanol (2.0 mL). The resulting mixture was stirred for 1 h at 100° C. The residue was purified by reverse phase flash column chromatography eluting with acetonitrile (20-100%) in water to yield the title compound as a yellow solid (100 mg, 84%).

(ES, m/z): [M+H]⁺=573.2

### 6-(2-chlorophenyl)-5-ethynyl-2-[(3-methoxyphenyl)amino]-8-methylpyrido[2,3-]pyrimidin-7-one

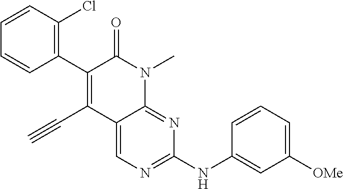 [SMILES: C#Cc1c(-c2ccccc2Cl)c(=O)n(C)c2nc(Nc3cccc(OC)c3)ncc12]

General procedure 15 was applied to 6-(2-chlorophenyl)-2-[(3-methoxyphenyl)amino]-8-methyl-5-[2-(triisopropylsilyl)ethynyl]pyrido[2,3-d]pyrimidin-7-one (100 mg, 0.174 mmol) and potassium fluoride (101 mg, 1.74 mmol) in methanol (3.0 mL). The resulting mixture was stirred for 2 h at 70° C. The residue was purified by reverse phase flash column chromatography eluting with acetonitrile (20-80%) in water to yield the title compound as a yellow solid (25.0 mg, 34%).

(ES, m/z): [M+H]⁺=417.1

¹H NMR (300 MHz, DMSO-d6) δ 10.32 (s, 1H), 8.91 (s, 1H), 7.64 (t, J=2.2 Hz, 1H), 7.60-7.54 (m, 1H), 7.47-7.33 (m, 4H), 7.26 (t, J=8.1 Hz, 1H), 6.65 (ddd, J=8.1, 2.5, 1.0 Hz, 1H), 4.92 (s, 1H), 3.79 (s, 3H), 3.67 (s, 3H).

### Example 132

### 6-(2-chlorophenyl)-2-{[3-(hydroxymethyl)phenyl]amino}-8-methyl-5-[2-(triisopropylsilyl)ethynyl]pyrido[2,3-d]pyrimidin-7-one

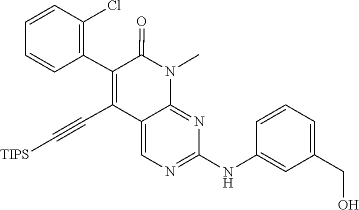

General procedure 13 was applied to 6-(2-chlorophenyl)-2-methanesulfonyl-8-methyl-5-[2-(triisopropylsilyl)ethynyl]pyrido[2,3-d]pyrimidin-7-one (110 mg, 0.207 mmol) with (3-aminophenyl)methanol (25.6 mg, 0.207 mmol) and trifluoroacetic acid (23.6 mg, 0.207 mmol) in 2-butanol (2.0 mL). The resulting mixture was stirred for 1 h at 100° C. The residue was purified by reverse phase flash column chromatography eluting with acetonitrile (20-100%) in water to yield the title compound as a yellow solid (100 mg, 84%).

(ES, m/z): [M+H]⁺=574.21

### 6-(2-chlorophenyl)-5-ethynyl-2-{[3-(hydroxymethyl)phenyl]amino}-8-methylpyrido[2,3-]pyrimidin-7-one

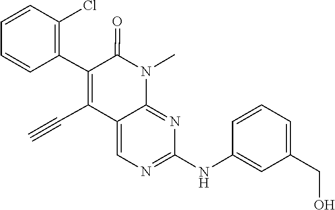 [SMILES: C#Cc1c(-c2ccccc2Cl)c(=O)n(C)c2nc(Nc3cccc(CO)c3)ncc12]

General procedure 15 was applied to 6-(2-chlorophenyl)-2-{[3-(hydroxymethyl)phenyl]amino}-8-methyl-5-[2-(triisopropylsilyl)ethynyl]pyrido[2,3-d]pyrimidin-7-one (100 mg, 0.174 mmol) and potassium fluoride (101 mg, 1.74 mmol) in methanol. The resulting mixture was stirred for 2 h at 60° C. The residue was purified by reverse phase flash column chromatography eluting with acetonitrile (20-80%) in water to yield the title compound as a yellow solid (25.0 mg, 34%).

(ES, m/z): [M+H]⁺=417.1

¹H NMR (300 MHz, DMSO-d6) δ 10.30 (s, 1H), 8.90 (s, 1H), 7.92 (s, 1H), 7.72-7.62 (m, 1H), 7.60-7.53 (m, 1H), 7.48-7.26 (m, 4H), 7.02 (d, J=7.5 Hz, 1H), 5.20 (t, J=5.7 Hz, 1H), 4.90 (s, 1H), 4.53 (d, J=5.7 Hz, 2H), 3.66 (s, 3H).

### Example 133

### 6-(2-chlorophenyl)-8-methyl-2-(methylsulfanyl)-5-[2-(triisopropylsilyl)ethynyl]pyrido[2,3-d]pyrimidin-7-one

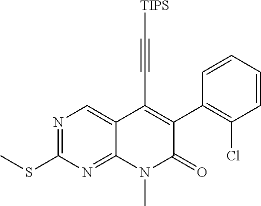

General procedure 17 was applied to 6-bromo-8-methyl-2-(methylsulfanyl)-5-[2-(triisopropylsilyl)ethynyl]pyrido[2,3-d]pyrimidin-7-one (800 mg, 1.65 mmol) with 2-chlorophenylboronic acid (386 mg, 2.47 mmol), potassium carbonate (455 mg, 3.29 mmol) and 1,1′-bis(diphenylphosphino)ferrocene)palladium(II) dichloride (120 mg, 0.165 mmol) in dioxane (8.0 mL) and water (3.6 mL). The residue was purified by flash column chromatography eluting with ethyl acetate (5%) in 40-60 petroleum ether to afford the title compound as a yellow solid (350 mg, 41%).

(ES, m/z): [M+H]⁺=498.2

### 6-(2-chlorophenyl)-2-methanesulfonyl-8-methyl-5-[2-(triisopropylsilyl)ethynyl]pyrido[2,3-d]pyrimidin-7-one

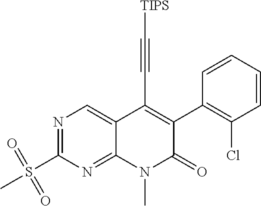

General procedure 10 was applied to 6-(2-chlorophenyl)-8-methyl-2-(methylsulfanyl)-5-[2-(triisopropylsilyl)ethynyl]pyrido[2,3-d]pyrimidin-7-one (350 mg, 0.674 mmol) and m-CPBA (291 mg, 1.68 mmol) in dichloromethane (3.5 mL) to yield the title compound as a yellow solid (250 mg, 67%).

(ES, m/z): [M+H]⁺=530.2

### 6-(2-chlorophenyl)-2-[(4-{[2-(dimethylamino)ethyl](methyl)amino}-3-methylphenyl)amino]-8-methyl-5-[2-(triisopropylsilyl)ethynyl]pyrido[2,3-d]pyrimidin-7-one

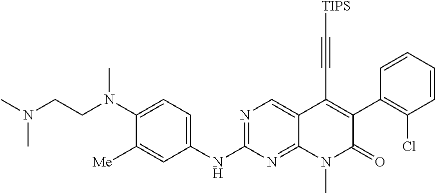

General procedure 13 was applied to 6-(2-chlorophenyl)-2-methanesulfonyl-8-methyl-5-[2-(triisopropylsilyl)ethynyl]pyrido[2,3-d]pyrimidin-7-one (250 mg, 0.453 mmol) with N¹-[2-(dimethylamino)ethyl]-N¹,N²-dimethylbenzene-1,4-diamine (113 mg, 0.544 mmol) and trifluoroacetic acid (103 mg, 0.906 mmol) in 2-butanol (2.5 mL). The solution was stirred for 2 h at 100° C. The residue was purified by reverse phase flash column chromatography eluting with acetonitrile (30-60%) in water (0.1% formic acid) to yield the title compound as an orange solid (100 mg, 33%).

(ES, m/z): [M+H]⁺=657.3

### 6-(2-chlorophenyl)-2-[(4-{[2-(dimethylamino)ethyl](methyl)amino}-3-methylphenyl)amino]-5-ethynyl-8-methylpyrido[2,3-d]pyrimidin-7-one

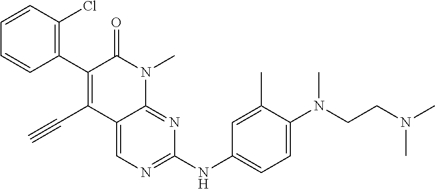 [SMILES: C#Cc1c(-c2ccccc2Cl)c(=O)n(C)c2nc(Nc3ccc(N(C)CCN(C)C)c(C)c3)ncc12]

General procedure 15 was applied to 6-(2-chlorophenyl)-2-[(4-{[2-(dimethylamino)ethyl](methyl)amino}-3-methylphenyl)amino]-8-methyl-5-[2-(triisopropylsilyl)ethynyl]pyrido[2,3-d]pyrimidin-7-one (100 mg, 0.152 mmol) and potassium fluoride (177 mg, 3.04 mmol) in THF (2.0 mL), methanol (1.0 mL) and water (1.0 mL). The solution was stirred for 2 h at 80° C. The residue was purified by reverse phase flash column chromatography eluting with acetonitrile (10-50%) in water (0.1% formic acid) to yield the title compound as a yellow solid (40.0 mg, 51%).

(ES, m/z): [M+H]⁺=501.2.

¹H NMR (400 MHz, DMSO-d₆) δ 10.17 (s, 1H), 8.86 (s, 1H), 7.63 (s, 2H), 7.59-7.51 (m, 1H), 7.49-7.40 (m, 2H), 7.40-7.34 (m, 1H), 7.10 (d, J=9.3 Hz, 1H), 4.90 (s, 1H), 3.64 (s, 3H), 2.95 (dd, J=8.5, 5.9 Hz, 2H), 2.63 (s, 3H), 2.45 (s, 2H), 2.26 (s, 3H), 2.20 (s, 6H).

### Example 134

### 6-(2,6-dichlorophenyl)-2-[(4-{[2-(dimethylamino)ethyl](methyl)amino}phenyl)amino]-8-methyl-5-[2-(triisopropylsilyl) ethynyl]pyrido[2,3-d]pyrimidin-7-one

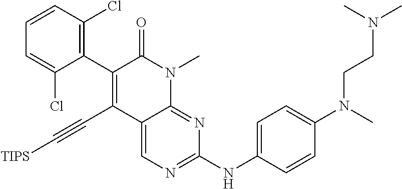

General procedure 13 was applied to 6-(2,6-dichlorophenyl)-2-methanesulfonyl-8-methyl-5-[2-(triisopropylsilyl)ethynyl]pyrido[2,3-d]pyrimidin-7-one (200 mg, 0.354 mmol) with N¹-[2-(dimethylamino)ethyl]-N¹-methylbenzene-1,4-diamine (103 mg, 0.531 mmol) and trifluoroacetic acid (60.6 mg, 0.531 mmol) in 2-butanol (3.0 mL). The reaction was stirred overnight at 100° C. The residue was purified by reverse phase flash column chromatography eluting with acetonitrile (50-100%) in water to yield the title compound as a red solid (100 mg, 42%).

(ES, m/z): [M+H]⁺=677.3

### 6-(2,6-dichlorophenyl)-2-[(4-{[2-(dimethylamino)ethyl](methyl)amino}phenyl)amino]-5-ethynyl-8-methylpyrido[2,3-d]pyrimidin-7-one

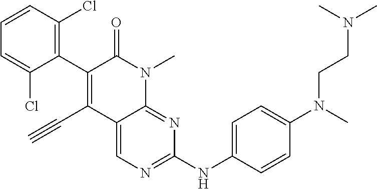 [SMILES: C#Cc1c(-c2c(Cl)cccc2Cl)c(=O)n(C)c2nc(Nc3ccc(N(C)CCN(C)C)cc3)ncc12]

General procedure 15 was applied to 6-(2,6-dichlorophenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one (100 mg, 0.148 mmol) with potassium fluoride (85.7 mg, 1.48 mmol) in THF (3.0 mL) and water (0.50 mL). The resulting solution was stirred overnight at 50° C. The crude product was purified by Prep-HPLC eluting with acetonitrile (50-68%) in water to yield the title compound as a red solid (20.0 mg, 26%) as a red solid.

(ES, m/z): [M+H]⁺=521.3

¹H NMR (400 MHz, DMSO-d₆) δ 10.14 (s, 1H), 8.83 (s, 1H), 7.72-7.53 (m, 4H), 7.53-7.43 (m, 1H), 6.71 (d, J=8.6 Hz, 2H), 3.63 (s, 3H), 3.40 (s, 2H), 2.89 (s, 3H), 2.37 (t, J=7.2 Hz, 2H), 2.18 (s, 6H).

### Example 135

### 6-bromo-8-methyl-2-(methylsulfanyl)-6H-pyrido[2,3-d]pyrimidine-5,7-dione

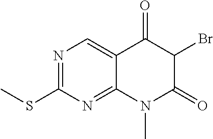 [SMILES: CSc1ncc2c(n1)N(C)C(=O)C(Br)C2=O]

A solution of 8-methyl-2-(methylsulfanyl)-6H-pyrido[2,3-d]pyrimidine-5,7-dione (24.0 g, 107 mmol, 1.0 eq.), and N-bromosuccinimide (22.9 g, 129 mmol, 1.2 eq.) in dichloromethane (240 mL) was stirred at room temperature for 2.5 h. The resulting solution was quenched with water (500 ml) and ethyl acetate (1.0 L). The organic layer was separated, dried (Na₂SO₄) and concentrated under reduced pressure. The residue was triturated with ethyl acetate and 40-60 petroleum ether (200 mL, 1:1). The resulting solid was collected by filtration, washing with 40-60 petroleum ether and then dried under vacuum to yield the title compound as a white solid (19.8 g, 61%).

### 6-bromo-8-methyl-2-(methylthio)-7-oxo-7,8-dihydropyrido[2,3-d]pyrimidin-5-yl trifluoromethanesulfonate

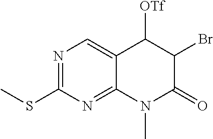 [SMILES: CSc1ncc2c(n1)N(C)C(=O)C(Br)C2OS(=O)(=O)C(F)(F)F]

General procedure 8 was applied to 6-bromo-8-methyl-2-(methylsulfanyl)-6H-pyrido[2,3-d]pyrimidine-5,7-dione (19.0 g, 62.9 mmol), triflic anhydride (39.0 g, 138 mmol) and triethylamine (12.7 g, 126 mmol) in dichloromethane (190 mL) to yield the title compound as a brown solid (20.5 g, 75%) which was used without any further purification.

### 6-bromo-8-methyl-2-(methylsulfanyl)-5-[2-(triisopropylsilyl)ethynyl]pyrido[2,3-d]pyrimidin-7-one

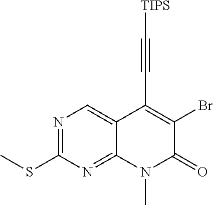

General procedure 9 was applied to 6-bromo-8-methyl-2-(methylsulfanyl)-7-oxopyrido[2,3-d]pyrimidin-5-yl trifluoromethanesulfonate (4.00 g, 9.21 mmol), triisopropylsilylacetylene (3.36 g, 18.4 mmol), copper(I) iodide (320 mg, 1.65 mmol), bis(triphenylphosphine)palladium(II) dichloride (650 mg, 0.921 mmol) and N,N-diisopropyl ethylamine (1.43 g, 11.0 mmol) in DMF (40 mL). The residue was purified by flash column chromatography eluting with ethyl acetate (0-25%) in 40-60 petroleum ether to yield the title compound (2.30 g, 53%).

### 6-(2-chloro-3-fluorophenyl)-8-methyl-2-(methylsulfanyl)-5-[2-(triisopropylsilyl)ethynyl]pyrido[2,3-d]pyrimidin-7-one

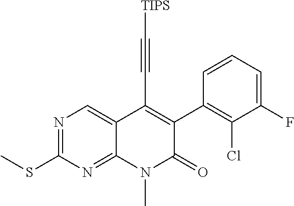

General procedure 17 was applied to 6-bromo-8-methyl-2-(methylsulfanyl)-5-[2-(triisopropylsilyl)ethynyl]pyrido[2,3-d]pyrimidin-7-one (600 mg, 1.29 mmol), 2-chloro-3-fluorophenylboronic acid (448. mg, 2.57 mmol), potassium carbonate (358 mg, 2.57 mmol), 1,1′-Bis(diphenylphosphino)ferrocene)palladium(II) dichloride (94.1 mg, 0.129 mmol) in dioxane (4.0 mL) and water (1.0 mL). The crude material was purified by flash column chromatography eluting with ethyl acetate (0-75%) in 40-60 petroleum ether to yield the title compound as a yellow solid (330 mg, 49.71%).

(ES, m/z): [M+H]⁺=516.2.

### 6-(2-chloro-3-fluorophenyl)-2-methanesulfonyl-8-methyl-5-[2-(triisopropylsilyl)ethynyl]pyrido[2,3-d]pyrimidin-7-one

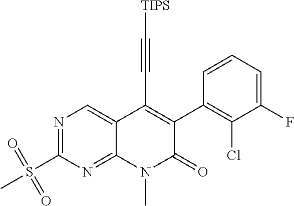

General procedure 10 was applied to 6-(2-chloro-3-fluorophenyl)-8-methyl-2-(methylsulfanyl)-5-[2-(triisopropylsilyl)ethynyl]pyrido[2,3-d]pyrimidin-7-one (330 mg, 0.639 mmol) to yield the title compound as a yellow solid (200 mg, 57%).

(ES, m/z): [M+H]⁺=548.2.

### 6-(2-chloro-3-fluorophenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one

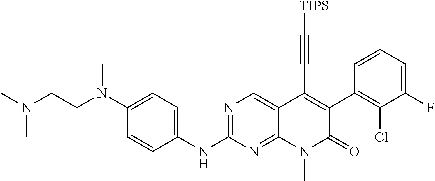

General procedure 13 was applied to 6-(2-chloro-3-fluorophenyl)-2-methanesulfonyl-8-methyl-5-[2-(triisopropylsilyl)ethynyl]pyrido[2,3-d]pyrimidin-7-one (200 mg, 0.365 mmol) with N¹-[2-(dimethylamino)ethyl]-N¹-methylbenzene-1,4-diamine (84.6 mg, 0.438 mmol) and trifluoroacetic acid (83.2 mg, 0.730 mmol) in 2-butanol (3.0 mL). The resulting solution was stirred for 3 h at 100° C. then diluted with methanol (10 mL) and applied onto a reverse phase column directly, eluting with acetonitrile (70-100%) in water (0.1% trifluoroacetic acid). The resulting solution was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a red solid (150 mg, 62%).

(ES, m/z): [M+H]⁺=661.3.

### 6-(2-chloro-3-fluorophenyl)-2-[(4-{[2-(dimethylamino)ethyl](methyl)amino}phenyl)amino]-5-ethynyl-8-methylpyrido[2,3-d]pyrimidin-7-one

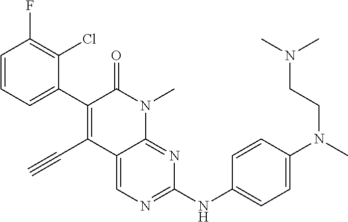 [SMILES: C#Cc1c(-c2cccc(F)c2Cl)c(=O)n(C)c2nc(Nc3ccc(N(C)CCN(C)C)cc3)ncc12]

General procedure 15 was applied to 6-(2-chloro-3-fluorophenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one (100 mg, 0.151 mmol) with potassium fluoride (87.8 mg, 1.51 mmol) in THF (0.50 mL) and methanol (0.50 mL). The resulting solution was stirred for 2 h at 70° C. before being purified by reverse phase flash column chromatography, eluting with acetonitrile (20-100%) in water (0.1% formic acid). The desired fractions were collected and concentrated under reduced pressure to remove most of the acetonitrile then lyophilized to yield the title compound as an orange solid (25.0 mg, 31%).

(ES, m/z): [M+H]⁺=505.2.

¹H NMR (400 MHz, DMSO-d6) δ 10.09 (s, 1H), 8.83 (s, 1H), 7.63 (s, 2H), 7.48 (d, J=12.0 Hz, 2H), 7.25 (s, 1H), 6.73 (d, J=8.8 Hz, 2H), 4.92 (s, 1H), 3.44 (s, 4H), 2.90 (s, 3H), 2.27 (s, 7H).

### Example 136

### 6-(2-chloro-5-methoxyphenyl)-8-methyl-2-(methylsulfanyl)-5-[2-(triisopropylsilyl)ethynyl]pyrido[2,3-d]pyrimidin-7-one

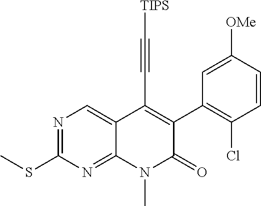

General procedure 17 was applied to 6-bromo-8-methyl-2-(methylsulfanyl)-5-[2-(triisopropylsilyl)ethynyl]pyrido[2,3-d]pyrimidin-7-one (600 mg, 1.22 mmol) with 2-chloro-5-methoxyphenylboronic acid (341 mg, 1.83 mmol), potassium carbonate (338 mg, 2.44 mmol) and 1,1′-Bis(diphenylphosphino)ferrocene)palladium(II) dichloride (89.4 mg, 0.122 mmol) in dioxane (6.0 mL) and water (1.5 mL). The residue was purified by flash column chromatography eluting with ethyl acetate (5%) in 40-60 petroleum ether to afford the title compound as a yellow solid (550 mg, 82%).

(ES, m/z): [M+H]⁺=528.2

### 6-(2-chloro-5-methoxyphenyl)-2-methanesulfonyl-8-methyl-5-[2-(triisopropylsilyl)ethynyl]pyrido[2,3-d]pyrimidin-7-one

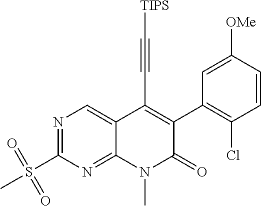

General procedure 10 was applied to 6-(2-chloro-5-methoxyphenyl)-8-methyl-2-(methylsulfanyl)-5-[2-(triisopropylsilyl)ethynyl]pyrido[2,3-d]pyrimidin-7-one (550 mg, 1.00 mmol) and m-CPBA (379 mg, 2.20 mmol) in dichloromethane (5.5 mL) to yield the tile compound as a yellow solid (260 mg, 44%).

(ES, m/z): [M+H]⁺=560.2

### 6-(2-chloro-5-methoxyphenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one

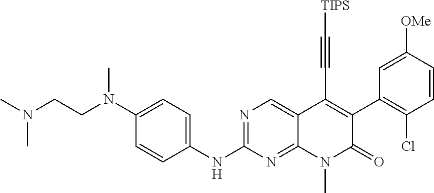

General procedure 13 was applied to 6-(2-chloro-5-methoxyphenyl)-2-methanesulfonyl-8-methyl-5-[2-(triisopropylsilyl)ethynyl]pyrido[2,3-d]pyrimidin-7-one (550 mg, 0.943 mmol) with N¹-[2-(dimethylamino)ethyl]-N¹-methylbenzene-1,4-diamine (218 mg, 1.13 mmol) and trifluoroacetic acid (215 mg, 1.88 mmol) in 2-butanol (5.5 mL). The solution mixture was stirred for 2 h at 100° C. The residue was purified by reverse phase flash column chromatography eluting with acetonitrile (30-60%) in water (0.1% formic acid) to yield the title compound as an orange solid (260 mg, 40%).

(ES, m/z): [M+H]⁺=673.4

### 6-(2-chloro-5-methoxyphenyl)-2-[(4-{[2-(dimethylamino)ethyl](methyl)amino}phenyl)amino]-5-ethynyl-8-methylpyrido[2,3-d]pyrimidin-7-one

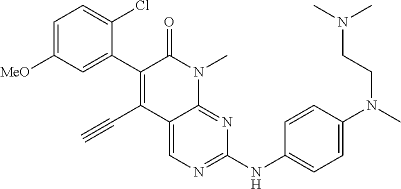 [SMILES: C#Cc1c(-c2cc(OC)ccc2Cl)c(=O)n(C)c2nc(Nc3ccc(N(C)CCN(C)C)cc3)ncc12]

General procedure 15 was applied to 6-(2-chloro-5-methoxyphenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one (230 mg, 0.331 mmol) with potassium fluoride (385 mg, 6.62 mmol) in THF (2.0 mL), methanol (1.0 mL) and water (0.05 mL). The resulting mixture was stirred for 2 h at 80° C. The residue was purified by reverse phase flash column chromatography eluting with acetonitrile (30-60%) in water to yield the title compound as an orange solid (79.0 mg, 45%).

(ES, m/z): [M+H]⁺=517.2.

¹H NMR (400 MHz, DMSO-d6) δ 10.06 (s, 1H), 8.81 (s, 1H), 7.62 (s, 2H), 7.45 (d, J=8.8 Hz, 1H), 7.01 (dd, J=8.8, 3.1 Hz, 1H), 6.94 (d, J=3.0 Hz, 1H), 6.71 (d, J=8.7 Hz, 2H), 4.89 (s, 1H), 3.77 (s, 3H), 3.61 (s, 3H), 3.41 (d, J=7.2 Hz, 2H), 2.89 (s, 3H), 2.38 (t, J=7.2 Hz, 2H), 2.18 (s, 6H).

### Example 137

### 6-(2-chloro-5-fluorophenyl)-8-methyl-2-(methylsulfanyl)-5-[2-(triisopropylsilyl)ethynyl]pyrido[2,3-d]pyrimidin-7-one

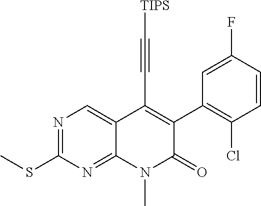

General procedure 17 was applied to 6-bromo-8-methyl-2-(methylsulfanyl)-5-[2-(triisopropylsilyl)ethynyl]pyrido[2,3-d]pyrimidin-7-one (600 mg, 1.29 mmol) with 2-chloro-5-fluorophenylboronic acid (448 mg, 2.57 mmol), potassium carbonate (358 mg, 2.57 mmol) and 1,1′-Bis(diphenylphosphino)ferrocene)palladium(II) dichloride (94.1 mg, 0.129 mmol) in dioxane (4 mL) and water (1.0 mL). The residue was purified by flash column chromatography eluting with ethyl acetate (0-75%) in 40-60 petroleum ether to yield the title compound as a yellow solid (440 mg, 66%).

(ES, m/z): [M+H]⁺=516.1.

### 6-(2-chloro-5-fluorophenyl)-2-methanesulfonyl-8-methyl-5-[2-(triisopropylsilyl)ethynyl]pyrido[2,3-d]pyrimidin-7-one

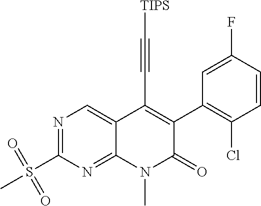

General procedure 10 was applied to 6-(2-chloro-5-fluorophenyl)-8-methyl-2-(methylsulfanyl)-5-[2-(triisopropylsilyl)ethynyl]pyrido [2,3-d]pyrimidin-7-one (440 mg, 0.852 mmol) and m-CPBA (323 mg, 1.87 mmol) in dichloromethane (5.0 mL) to yield the title compound as a yellow solid (300 mg, 64%) which was used without any purification.

(ES, m/z): [M+H]⁺=548.1.

### 6-(2-chloro-5-fluorophenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one

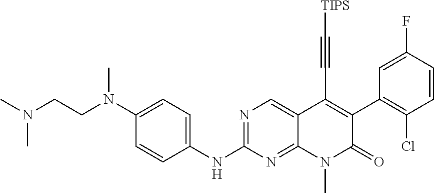

General procedure 13 was applied to 6-(2-chloro-5-fluorophenyl)-2-methanesulfonyl-8-methyl-5-[2-(triisopropylsilyl)ethynyl]pyrido [2,3-d]pyrimidin-7-one (300 mg, 0.547 mmol) with trifluoroacetic acid (125 mg, 1.09 mmol) and N¹-[2-(dimethylamino)ethyl]-N¹-methylbenzene-1,4-diamine (127 mg, 0.656 mmol) in 2-butanol (5 mL). The resulting solution was stirred for 3 h at 100° C. before being purified by reverse phase flash column chromatography, eluting with acetonitrile (70-100%) in water (0.1% trifluoroacetic acid). The mixture was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a red solid (200 mg, 55%).

(ES, m/z): [M+H]⁺=661.3.

### 6-(2-chloro-5-fluorophenyl)-2-[(4-{[2-(dimethylamino)ethyl](methyl)amino}phenyl)amino]-5-ethynyl-8-methylpyrido[2,3-d]pyrimidin-7-one

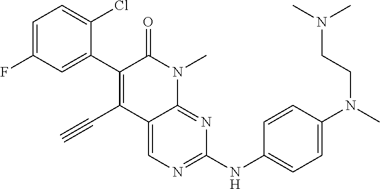 [SMILES: C#Cc1c(-c2cc(F)ccc2Cl)c(=O)n(C)c2nc(Nc3ccc(N(C)CCN(C)C)cc3)ncc12]

General procedure 15 was applied to 6-(2-chloro-5-fluorophenyl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one (200 mg, 0.302 mmol) with potassium fluoride (176 mg, 3.02 mmol) in THF (1.0 mL) and methanol (0.50 mL). The resulting solution was stirred for 2 h at 70° C. before being purified by reverse phase flash column chromatography, eluting with acetonitrile (20-100%) in water (0.1% formic acid). The desired fractions were collected and concentrated under reduced pressure to remove most of the acetonitrile then lyophilized to yield the title compound as an orange solid (77.2 mg, 48%).

(ES, m/z): [M+H]⁺=505.2.

¹H NMR (400 MHz, DMSO-d6) δ 10.09 (s, 1H), 8.83 (s, 1H), 7.63 (s, 2H), 7.48 (d, J=12.0 Hz, 3H), 7.25 (s, 1H), 6.73 (d, J=8.8 Hz, 2H), 4.92 (s, 1H), 3.44 (s, 4H), 2.90 (s, 3H), 2.27 (s, 7H).

### Example 138

### 6-(2-methoxyphenyl)-8-methyl-2-(methylsulfanyl)-5-[2-(triisopropylsilyl)ethynyl]pyrido[2,3-d]pyrimidin-7-one

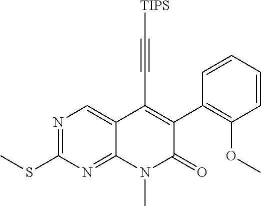

General procedure 17 was applied to 6-bromo-8-methyl-2-sulfanyl-5-[2-(triisopropylsilyl)ethynyl]pyrido[2,3-d]pyrimidin-7-one (1.60 g, 3.54 mmol) with 2-methoxyphenylboronic acid (1.07 g, 7.07 mmol), potassium carbonate (0.980 g, 7.07 mmol) and 1,1′-bis(diphenylphosphino)ferrocene)palladium(II) dichloride (0.260 g, 0.354 mmol), dioxane (8 mL) and water (2.0 mL). The residue was purified by flash column chromatography eluting with ethyl acetate (0-75%) in 40-60 petroleum ether to yield the title compound as a yellow solid (1.00 g, 57%).

(ES, m/z): [M+H]⁺=494.2.

### 2-methanesulfonyl-6-(2-methoxyphenyl)-8-methyl-5-[2-(triisopropylsilyl)ethynyl]pyrido[2,3-d]pyrimidin-7-one

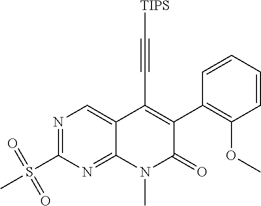

General procedure 10 was applied to 6-(2-methoxyphenyl)-8-methyl-2-(methylsulfanyl)-5-[2-(triisopropylsilyl)ethynyl]pyrido [2,3-d]pyrimidin-7-one (1.00 g, 2.025 mmol) and m-CPBA (769 mg, 4.45 mmol) in dichloromethane (10 mL) to yield the title compound yellow solid (700 mg, 66%).

(ES, m/z): [M+H]⁺=526.2.

### 2-[(4-{[2-(dimethylamino)ethyl](methyl)amino}phenyl)amino]-6-(2-methoxyphenyl)-8-methyl-5-[2-(triisopropylsilyl)ethynyl]pyrido[2,3-d]pyrimidin-7-one

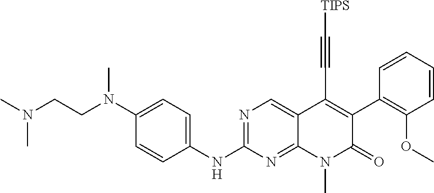

General procedure 13 was applied to 2-methanesulfonyl-6-(2-methoxyphenyl)-8-methyl-5-[2-(triisopropylsilyl)ethynyl]pyrido[2,3-d]pyrimidin-7-one (550 mg, 1.05 mmol), N¹-[2-(dimethylamino)ethyl]-N¹-methylbenzene-1,4-diamine (243 mg, 1.25 mmol), trifluoroacetic acid (238 mg, 2.09 mmol) in 2-butanol. The resulting solution was stirred for 3 h at 100° C. before being purified by reverse phase flash column chromatography, eluting with acetonitrile (70-100%) in water (0.1% trifluoroacetic acid). The mixture was basified to pH 8 with saturated aqueous sodium hydrogen carbonate solution and extracted with ethyl acetate (3×200 mL).

The combined organic layers were washed with brine (2×200 mL), dried (Na₂SO₄) and concentrated under reduced pressure to yield the title compound as a red solid (300 mg, 45%).

(ES, m/z): [M+H]⁺=639.3.

### 2-[(4-{[2-(dimethylamino)ethyl](methyl)amino}phenyl)amino]-5-ethynyl-6-(2-methoxyphenyl)-8-methylpyrido [2,3-d]pyrimidin-7-one

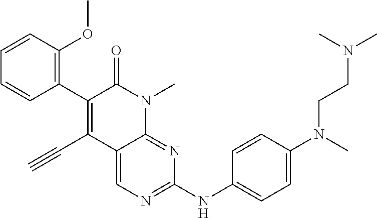 [SMILES: C#Cc1c(-c2ccccc2OC)c(=O)n(C)c2nc(Nc3ccc(N(C)CCN(C)C)cc3)ncc12]

General procedure 15 was applied to 2-[(4-{[2-(dimethylamino)ethyl](methyl)amino}phenyl)amino]-6-(2-methoxyphenyl)-8-methyl-5-[2-(triisopropylsilyl)ethynyl]pyrido[2,3-d]pyrimidin-7-one (300 mg, 0.470 mmol) and potassium fluoride (273 mg, 4.70 mmol) in THF (1.5 mL) and methanol (1.5 mL). The resulting solution was stirred for 2 h at 70° C. before being purified by reverse phase flash column chromatography, eluting with acetonitrile (20-100%) in water (0.1% formic acid). The desired fractions were collected and concentrated under reduced pressure to remove most of the acetonitrile then lyophilized to yield the title compound as an orange solid (150 mg, 66%).

(ES, m/z): [M+H]⁺=483.3.

¹H NMR (400 MHz, DMSO-d6) δ 9.95 (s, 1H), 8.78 (s, 1H), 8.22 (s, 1H), 7.61 (s, 2H), 7.35 (s, 1H), 7.16 (s, 1H), 7.08 (s, 1H), 7.00 (s, 1H), 6.71 (s, 2H), 4.77 (s, 1H), 3.71 (s, 3H), 3.60 (s, 3H), 3.40 (s, 2H), 2.90 (s, 3H), 2.47 (s, 2H), 2.25 (s, 6H).

### Example 139

### 6-(3-chloropyridin-4-yl)-8-methyl-2-(methylsulfanyl)-5-[2-(triisopropylsilyl)ethynyl]pyrido[2,3-d]pyrimidin-7-one

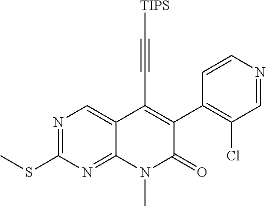

General procedure 17 was applied to 6-bromo-8-methyl-2-(methylsulfanyl)-5-[2-(triisopropylsilyl)ethynyl]pyrido[2,3-d]pyrimidin-7-one (1.00 g, 2.14 mmol) and 3-chloropyridin-4-ylboronic acid (1.69 g, 10.7 mmol) in dioxane (4 mL) and water (1.0 mL). The residue was purified by flash column chromatography eluting with ethyl acetate (0-75%) in 40-60 petroleum ether to yield the title compound as a yellow solid (430 mg, 40%).

(ES, m/z): [M+H]⁺=499.2

### 6-(3-chloropyridin-4-yl)-2-methanesulfonyl-8-methyl-5-[2-(triisopropylsilyl)ethynyl]pyrido[2,3-d]pyrimidin-7-one

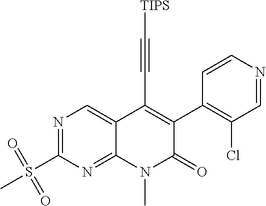

General procedure 10 was applied to 6-(3-chloropyridin-4-yl)-8-methyl-2-(methylsulfanyl)-5-[2-(triisopropylsilyl)ethynyl]pyrido [2,3-d]pyrimidin-7-one (430 mg, 0.861 mmol) and m-CPBA (327 mg, 1.89 mmol) in dichloromethane (10 mL) to yield the title compound as a yellow solid (330 mg, 72%).

(ES, m/z): [M+H]⁺=531.2

### 6-(3-chloropyridin-4-yl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one

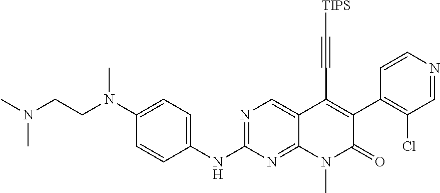

General procedure 13 was applied to 6-(3-chloropyridin-4-yl)-2-methanesulfonyl-8-methyl-5-[2-(triisopropylsilyl)ethynyl]pyrido[2,3-d]pyrimidin-7-one (330 mg, 0.621 mmol), N¹-[2-(dimethylamino)ethyl]-N¹-methylbenzene-1,4-diamine (144 mg, 0.745 mmol), trifluoroacetic acid (142 mg, 1.24 mmol) in 2-butanol. The resulting solution was stirred for 3 h at 100° C. before being purified by reverse phase flash column chromatography, eluting with acetonitrile (70-100%) in water (0.1% trifluoroacetic acid). The mixture was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a red solid (200 mg, 75%).

(ES, m/z): [M+H]⁺=644.3

### 6-(3-chloropyridin-4-yl)-2-[(4-{[2-(dimethylamino)ethyl](methyl)amino}phenyl)amino]-5-ethynyl-8-methylpyrido [2,3-d]pyrimidin-7-one

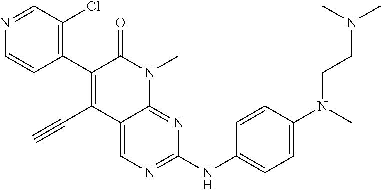 [SMILES: C#Cc1c(-c2ccncc2Cl)c(=O)n(C)c2nc(Nc3ccc(N(C)CCN(C)C)cc3)ncc12]

General procedure 15 was applied to 6-(3-chloropyridin-4-yl)-2-[(4-{[2-(dimethylamino)ethyl](methyl)amino}phenyl)amino]-8-methyl-5-[2-(triisopropylsilyl)ethynyl]pyrido[2,3-d]pyrimidin-7-one (200 mg, 0.310 mmol), potassium fluoride (180 mg, 3.10 mmol) in THF (1.0 mL) and methanol (1.0 mL). The resulting solution was stirred for 2 h at 50° C. before being purified by reverse phase flash column chromatography, eluting with acetonitrile (20-100%) in water (0.1% formic acid). The desired fractions were collected and concentrated under reduced pressure to remove most of the acetonitrile then lyophilized to dryness to yield the title compound as a red solid (30.0 mg, 13%).

(ES, m/z): [M+H]⁺=488.4.

¹H NMR (400 MHz, DMSO-d6) δ 10.13 (s, 1H), 8.81 (d, J=18.3 Hz, 2H), 8.62 (s, 1H), 7.74-7.41 (m, 3H), 6.71 (s, 2H), 4.98 (s, 1H), 3.62 (s, 3H), 3.39 (s, 2H), 2.90 (s, 3H), 2.40 (s, 2H), 2.20 (s, 6H).

### Example 140

### 6-benzyl-8-methyl-2-(methylsulfanyl)-6H-pyrido[2,3-d]pyrimidine-5,7-dione

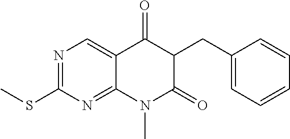 [SMILES: CSc1ncc2c(n1)N(C)C(=O)C(Cc1ccccc1)C2=O]

General procedure 7 was applied to 4-(methylamino)-2-(methylsulfanyl)pyrimidine-5-carboxylate (5.00 g, 22.0 mmol), sodium hydride (2.64 g, 110 mmol) and phenylacetyl chloride (7.42 g, 44.0 mmol) in DMF (50 mL) to yield the title compound as a yellow solid (3.00 g, 43%).

(ES, m/z): [M+H]⁺=314.1

### 6-benzyl-8-methyl-2-(methylsulfanyl)-7-oxopyrido[2,3-d]pyrimidin-5-yl trifluoromethanesulfonate

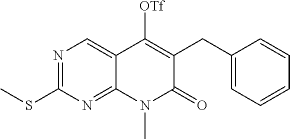 [SMILES: CSc1ncc2c(OS(=O)(=O)C(F)(F)F)c(Cc3ccccc3)c(=O)n(C)c2n1]

General procedure 8 was applied to 6-benzyl-8-methyl-2-(methylsulfanyl)-6H-pyrido[2,3-d]pyrimidine-5,7-dione (2.00 g, 6.38 mmol) with triflic anhydride (2.70 g, 9.57 mmol) and triethylamine (1.94 g, 19.1 mmol) in dichloromethane (20 mL). The residue was purified by flash column chromatography eluting with ethyl acetate (15%) in 40-60 petroleum ether to yield the title compound as a light yellow solid (1.50 g, 53%).

(ES, m/z): [M+H]⁺=446.0

### 6-benzyl-8-methyl-2-(methylsulfanyl)-5-[2-(triisopropylsilyl)ethynyl]pyrido[2,3-d]pyrimidin-7-one

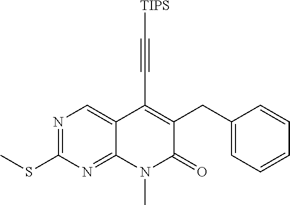

General procedure 9 was applied to 6-benzyl-8-methyl-2-(methylsulfanyl)-7-oxopyrido[2,3-d]pyrimidin-5-yl trifluoromethanesulfonate (1.50 g, 3.37 mmol), triisopropylsilylacetylene (1.23 g, 6.73 mmol), copper(I) iodide (64.1 mg, 0.337 mmol), bis(triphenylphosphine)palladium(II) dichloride (236 mg, 0.337 mmol) in DMF (30 mL) and N,N-diisopropylethylamine (1.31 g, 10.1 mmol). The crude material was purified by flash column chromatography eluting with 40-60 petroleum ether to yield the title compound as a light yellow solid (1.40 g, 81%).

(ES, m/z): [M+H]⁺=478.2

### 6-benzyl-8-methyl-2-(methylsulfanyl)-5-[2-(triisopropylsilyl)ethynyl]pyrido[2,3-d]pyrimidin-7-one

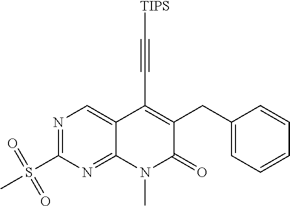

General procedure 10 was applied to 6-benzyl-8-methyl-2-(methylsulfanyl)-5-[2-(triisopropylsilyl)ethynyl]pyrido[2,3-d]pyrimidin-7-one (1.30 g, 2.72 mmol) and m-CPBA (1.41 g, 8.16 mmol) in dichloromethane (10 mL) to yield the title compound (1.30 g, 2.72 mmol) as a light yellow solid.

(ES, m/z): [M+H]⁺=510.2

### 6-benzyl-2-[(4-{[2-(dimethylamino)ethyl](methyl)amino}-2-methoxyphenyl)amino]-8-methyl-5-[2-(triisopropylsilyl)ethynyl]pyrido [2,3-d]pyrimidin-7-one

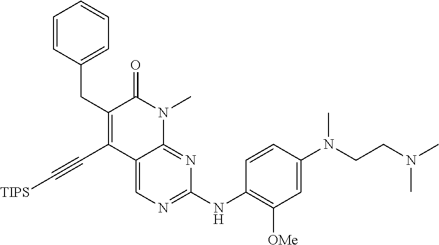

General procedure 13 was applied to 6-benzyl-2-methanesulfonyl-8-methyl-5-[2-(triisopropylsilyl)ethynyl]pyrido[2,3-d]pyrimidin-7-one (300 mg, 0.589 mmol), N¹-[2-(dimethylamino)ethyl]-3-methoxy-N¹-methylbenzene-1,4-diamine (197 mg, 0.883 mmol) and trifluoroacetic acid (100 mg, 0.883 mmol) in 2-butanol (10 mL). The resulting solution was stirred at 100° C. The crude product was purified by Prep-HPLC eluting with methanol (22-65%) in water (0.05% trifluoroacetic acid) to yield the title compound as a red solid (100 mg, 26%).

(ES, m/z): [M+H]⁺=653.4

### 6-benzyl-2-[(4-[[2-(dimethylamino)ethyl](methyl)amino]-2-methoxyphenyl)amino]-5-ethynyl-8-methylpyrido[2,3-d]pyrimidin-7-one

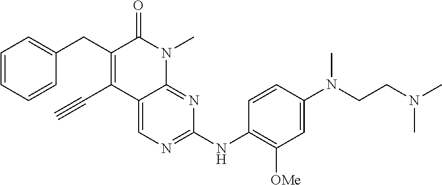 [SMILES: C#Cc1c(Cc2ccccc2)c(=O)n(C)c2nc(Nc3ccc(N(C)CCN(C)C)cc3OC)ncc12]

A solution of 6-benzyl-2-[(4-[[2-(dimethylamino)ethyl](methyl)amino]-2-methoxyphenyl)amino]-8-methyl-5-[2-(triisopropylsilyl)ethynyl]pyrido[2,3-d]pyrimidin-7-one (100 mg, 0.153 mmol, 1.0 eq.) and TBAF (32.0 mg, 0.122 mmol, 0.80 eq.) in THF (3.0 mL) was stirred for 20 min at room temperature. The reaction was then quenched by the addition of water (4.0 mL), extracted with ethyl acetate (2×5 mL) and the combined organic layers concentrated under reduced pressure. The crude product was purified by Prep-HPLC eluting with acetonitrile (30-70%) in water (0.05% formic acid) to yield the title compound as a red solid (20.0 mg, 26%).

(ES, m/z): [M+H]⁺=497.3

¹H NMR (300 MHz, CDCl₃) δ 8.88 (s, 1H), 8.24 (d, J=8.8 Hz, 1H), 7.72 (s, 1H), 7.52-7.42 (m, 2H), 7.31 (d, J=1.2 Hz, 1H), 7.26 (d, J=1.5 Hz, 1H), 7.20 (dd, J=8.4, 6.1 Hz, 1H), 6.40 (dd, J=6.5, 2.9 Hz, 2H), 4.20 (s, 2H), 3.93 (s, 3H), 3.87 (s, 1H), 3.73 (s, 3H), 3.50 (t, J=7.5 Hz, 2H), 3.00 (s, 3H), 2.55 (t, J=7.4 Hz, 2H), 2.35 (s, 6H).

### Example 141

### Methyl 5-hydroxy-2-(methylsulfanyl)-7-oxo-8-phenylpyrido[2,3-d]pyrimidine-6-carboxylate

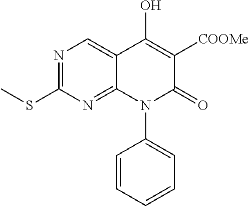 [SMILES: COC(=O)c1c(O)c2cnc(SC)nc2n(-c2ccccc2)c1=O]

To a solution of dimethyl malonate (1.64 g, 12.4 mmol, 1.5 eq.) in THF (30 mL) was added sodium hydride (400 mg, 60% in oil) at 0° C. The mixture was stirred for 15 min then 5-(1,2,3-benzotriazole-1-carbonyl)-2-(methylsulfanyl)-N-phenylpyrimidin-4-amine (3.00 g, 8.28 mmol) in THF (20 mL) was added. The resulting solution was stirred for 1 h at 0° C. then quenched with ice-water (100 mL). The pH value of the solution was adjusted to 3 with 3M hydrochloric acid aqueous solution. The solids were collected by filtration. The solids were dried in a vacuum oven to yield the title compound as a light yellow solid. (2.70 g, 95%).

### Methyl 2-(methylsulfanyl)-7-oxo-8-phenyl-5-(trifluoromethanesulfonyloxy)pyrido[2,3-d]pyrimidine-6-carboxylate

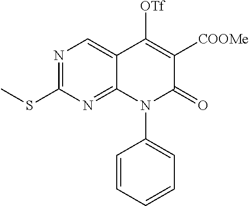 [SMILES: COC(=O)c1c(OS(=O)(=O)C(F)(F)F)c2cnc(SC)nc2n(-c2ccccc2)c1=O]

General procedure 8 was applied to methyl 5-hydroxy-2-(methylsulfanyl)-7-oxo-8-phenylpyrido[2,3-d]pyrimidine-6-carboxylate (2.00 g, 5.82 mmol), triethylamine (1.47 g, 14.6 mmol) and triflic anhydride (2.47 g, 8.77 mmol) in dichloromethane (20 mL). The residue was purified by flash column chromatography eluting with ethyl acetate (0-50%) in 40-60 petroleum ether to yield the title compound as a yellow solid (1.10 g, 40%).

### Methyl 2-(methylsulfanyl)-7-oxo-8-phenyl-5-[2-(triisopropylsilyl)ethynyl]pyrido[2,3-d]pyrimidine-6-carboxylate

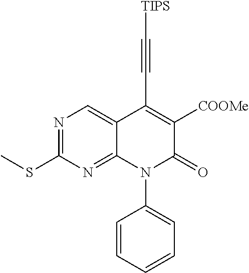

General procedure 9 was applied to methyl 2-(methylsulfanyl)-7-oxo-8-phenyl-5-(trifluoromethanesulfonyloxy)pyrido[2,3-d]pyrimidine-6-carboxylate (1.10 g, 2.31 mmol), triisopropylsilylacetylene (844 mg, 4.63 mmol), copper(I) iodide (44.1 mg, 0.231 mmol), bis(triphenylphsophine)palladium(II) dichloride (162 mg, 0.231 mmol) in DMF (10 mL) and N,N-diisopropylethylamine (5.0 mL). The residue was purified by flash column chromatography eluting with ethyl acetate (0-75%) in 40-60 petroleum ether to yield the title compound as a brown solid (1.00 g, 85%).

### 2-(methylsulfanyl)-7-oxo-8-phenyl-5-[2-(triisopropylsilyl) ethynyl]pyrido[2,3-d]pyrimidine-6-carboxylic acid

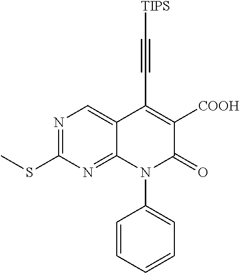

A solution of methyl 2-(methylsulfanyl)-7-oxo-8-phenyl-5-[2-(triisopropylsilyl)ethynyl]pyrido[2,3-d]pyrimidine-6-carboxylate (600 mg, 1.18 mmol, 1.0 eq.) in acetic acid (5.0 mL) and 3N aqueous hydrochloric acid (5.0 mL) was stirred for 5 h at 100° C. The reaction mixture was cooled to room temperature before being purified by reverse phase flash column chromatography, eluting with acetonitrile (30-100%) in water (0.1% NH₄HCO₃) to yield the title compound as a yellow solid (300 mg, 51%).

### N-methyl-2-(methylsulfanyl)-7-oxo-8-phenyl-5-[2-(triisopropylsilyl)ethynyl]pyrido[2,3-d]pyrimidine-6-carboxamide

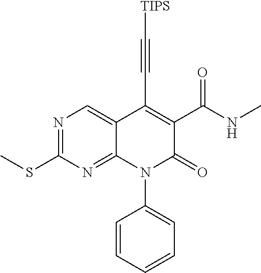

A solution of 2-(methylsulfanyl)-7-oxo-8-phenyl-5-[2-(triisopropylsilyl)ethynyl]pyrido[2,3-d]pyrimidine-6-carboxylic acid (300 mg, 0.608 mmol, 1.0 eq.), HATU (277 mg, 0.729 mmol, 1.2 eq.) and methyl amine (20.7 mg, 0.668 mmol, 1.1 eq.) in DMF (3.00 mL) was stirred for 2 h at room temperature. The resulting solution was diluted with water (20 mL) and extracted with dichloromethane (2×20 mL). The combined organic layers were washed with brine (2×20 mL), dried (Na₂SO₄) and concentrated under reduced pressure. The residue was purified by flash column chromatography eluting with ethyl acetate (50%) in 40-60 petroleum ether (1:1) to yield the title compound as a light yellow solid (200 mg, 65%).

### 2-methanesulfonyl-N-methyl-7-oxo-8-phenyl-5-[2-(triisopropylsilyl)ethynyl]pyrido[2,3-d]pyrimidine-6-carboxamide

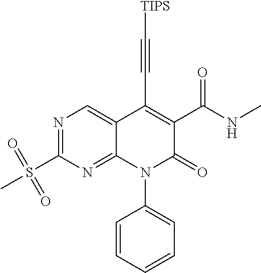

General procedure 10 was applied to N-methyl-2-(methylsulfanyl)-7-oxo-8-phenyl-5-[2-(triisopropylsilyl) ethynyl]pyrido[2,3-d]pyrimidine-6-carboxamide (200 mg, 0.395 mmol) with m-CPBA (136 mg, 0.789 mmol) in dichloromethane (3.0 mL) before being purified by reverse phase flash column chromatography, eluting with acetonitrile (10-100%) in water (0.1% NH₄HCO₃) to yield the title compound as a yellow solid (100 mg, 47%). 2-[(2-methoxyphenyl)amino]-N-methyl-7-oxo-8-phenyl-5-[2-(triisopropylsilyl)ethynyl]pyrido [2,3-d]pyrimidine-6-carboxamide

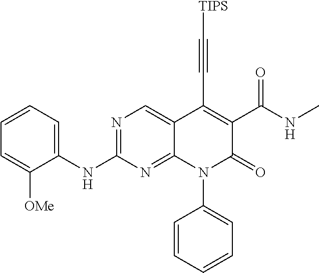

General procedure 13 was applied to 2-methanesulfonyl-N-methyl-7-oxo-8-phenyl-5-[2-(triisopropylsilyl)ethynyl]pyrido[2,3-d]pyrimidine-6-carboxamide (100 mg, 0.186 mmol), o-anisidine (22.8 mg, 0.186 mmol) and trifluoroacetic acid (21.2 mg, 0.186 mmol) in 2-butanol (1.0 mL). The reaction mixture was stirred at 110° C. for 1 h. The reaction mixture was cooled to room temperature before being purified by reverse phase flash column chromatography, eluting with acetonitrile (40-100%) in water (0.1% NH₄HCO₃) to yield the title compound as a yellow solid (50.0 mg, 46%).

### 5-ethynyl-2-[(2-methoxyphenyl)amino]-N-methyl-7-oxo-8-phenylpyrido[2,3-d]pyrimidine-6-carboxamide

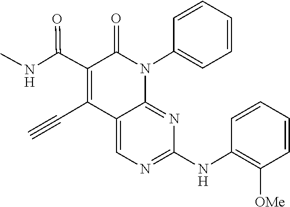 [SMILES: C#Cc1c(C(=O)NC)c(=O)n(-c2ccccc2)c2nc(Nc3ccccc3OC)ncc12]

General procedures 15 was applied to 2-[(2-methoxyphenyl)amino]-N-methyl-7-oxo-8-phenyl-5-[2-(triisopropylsilyl)ethynyl]pyrido[2,3-d]pyrimidine-6-carboxamide (50.0 mg, 0.086 mmol) and potassium fluoride (49.9 mg, 0.859 mmol) in THF (2. 0 mL) and water (0.50 mL). The resulting solution was stirred for 12 h at room temperature before being purified by reverse phase flash column chromatography, eluting with acetonitrile (30-100%) in water (0.1% NH₄HCO₃) to yield the title compound as a light yellow solid (7.00 mg, 19%).

(ES, m/z): [M+H]⁺=426

¹H-NMR (300 MHz, CDCl₃) δ 9.27 (s, 1H), 9.08 (s, 1H), 8.26 (s, 1H), 7.74-7.62 (m, 3H), 7.50-7.47 (m, 1H), 7.33-7.31 (m, 2H), 6.97-6.92 (m, 1H), 6.84 (d, J=8.0 Hz, 1H), 6.50 (s, 1H), 4.39 (s, 1H), 3.90 (s, 3H), 3.03-2.90 (m, 3H).

### Example 142

### Methyl 2-methanesulfonyl-7-oxo-8-phenyl-5-[2-(triisopropylsilyl)ethynyl]pyrido[2,3-d]pyrimidine-6-carboxylate

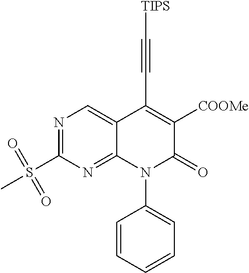

General procedure 10 was applied to methyl 2-(methylsulfanyl)-7-oxo-8-phenyl-5-[2-(triisopropylsilyl)ethynyl]pyrido[2,3-d]pyrimidine-6-carboxylate (2.80 g, 5.52 mmol) and m-CPBA (1.90 g, 11.0 mmol) in dichloromethane (30 mL) before being purified by reverse phase flash column chromatography, eluting with acetonitrile (10-100%) in water (0.1% NH₄HCO₃) to yield the title compound as a light yellow solid (1.50 g, 50%).

### Methyl 2-[(2-methoxyphenyl)amino]-7-oxo-8-phenyl-5-[2-(triisopropylsilyl)ethynyl]pyrido[2,3-d]pyrimidine-6-carboxylate

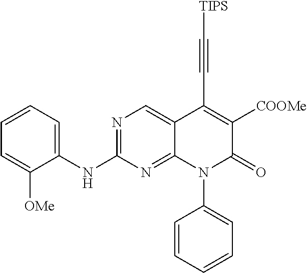

General procedure 13 was applied to methyl 2-methanesulfonyl-7-oxo-8-phenyl-5-[2-(triisopropylsilyl)ethynyl]pyrido[2,3-d]pyrimidine-6-carboxylate (1.50 g, 2.78 mmol), o-anisidine (342 mg, 2.78 mmol), trifluoroacetic acid (317 mg, 2.78 mmol) and 2-butanol (15.0 mL). The reaction mixture was stirred at 110° C. for 2 h. The reaction mixture was cooled to room temperature before being purified by reverse phase flash column chromatography, eluting with acetonitrile (10-100%) in water (0.1% NH₄HCO₃) to yield the title compound as a yellow solid (1.00 g, 62%).

### 2-[(2-methoxyphenyl)amino]-7-oxo-8-phenyl-5-[2-(triisopropylsilyl)ethynyl]pyrido[2,3-d]pyrimidine-6-carboxylic acid

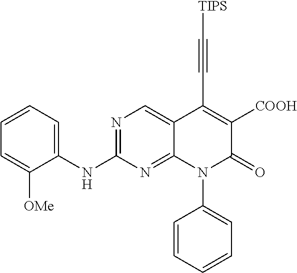

A solution of methyl 2-[(2-methoxyphenyl)amino]-7-oxo-8-phenyl-5-[2-(triisopropylsilyl)ethynyl]pyrido[2,3-d]pyrimidine-6-carboxylate (1.00 g, 1.72 mmol, 1.0 eq.) in 4M aqueous hydrochloric acid (5.0 mL) and acetic acid (10 mL) was stirred for 4 h at 100° C.

The reaction mixture was cooled to room temperature before being purified by reverse phase flash column chromatography, eluting with acetonitrile (30-100%) in water (0.1% trifluoroacetic acid) to yield the title compound as a yellow solid (600 mg, 61%).

### 6-amino-2-[(2-methoxyphenyl)amino]-8-phenyl-5-[2-(triisopropylsilyl)ethynyl]pyrido[2,3-d]pyrimidin-7-one

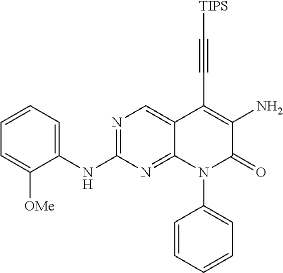

To a stirred solution of 2-[(2-methoxyphenyl)amino]-7-oxo-8-phenyl-5-[2-(triisopropylsilyl)ethynyl]pyrido[2,3-d]pyrimidine-6-carboxylic acid (600 mg, 1.05 mmol, 1.0 eq.) and triethylamine (139 mg, 1.37 mmol, 1.3 eq.) in THF (6.0 mL) was added DPPA (348 mg, 1.27 mmol, 1.2 eq.) dropwise and the reaction mixture was stirred for 1 h at 70° C. Water (1.00 mL, 2.11 mmol, 2.0 eq.) was added and the reaction mixture was refluxed for 1 h. The reaction mixture was cooled to room temperature before being purified by reverse phase flash column chromatography, eluting with acetonitrile (40-100%) in water (0.1% NH₄HCO₃) to yield the title compound as a light yellow solid (400 mg, 70%).

### 6-amino-5-ethynyl-2-[(2-methoxyphenyl)amino]-8-phenylpyrido[2,3-d]pyrimidin-7-one

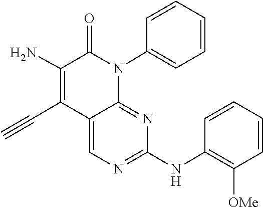 [SMILES: C#Cc1c(N)c(=O)n(-c2ccccc2)c2nc(Nc3ccccc3OC)ncc12]

General procedure 15 was applied to 6-amino-2-[(2-methoxyphenyl)amino]-8-phenyl-5-[2-(triisopropylsilyl) ethynyl]pyrido[2,3-d]pyrimidin-7-one (100 mg, 0.185 mmol) and potassium fluoride (107 mg, 1.85 mmol) in water (0.20 mL) and THF (1.0 mL). The resulting solution was stirred for 12 h at room temperature. The residue was purified by reverse phase flash column chromatography eluting with acetonitrile (35-100%) in water (0.1%, NH₄HCO₃) to yield the title compound as an off-white solid (20.0 mg, 28%).

(ES, m/z): [M+H]⁺=384

¹H-NMR (300 MHz, CDCl₃) δ 8.79 (s, 1H), 7.99 (s, 1H), 7.70-7.58 (m, 3H), 7.53 (d, J=8.1 Hz, 1H), 7.34 (dd, J=7.7, 1.9 Hz, 2H), 6.92-6.81 (m, 1H), 6.86-6.77 (m, 1H), 6.51 (t, J=7.8 Hz, 1H), 5.10 (s, 2H), 3.97 (s, 1H), 3.88 (s, 3H).

### Example 143

### N-[2-[(2-methoxyphenyl)amino]-7-oxo-8-phenyl-5-[2-(triisopropylsilyl)ethynyl]pyrido[2,3-d]pyrimidin-6-yl]acetamide

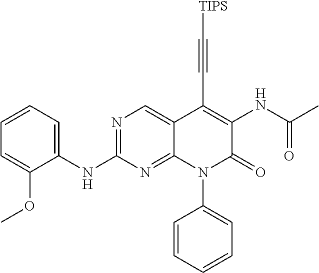

A solution of 6-amino-2-[(2-methoxyphenyl)amino]-8-phenyl-5-[2-(triisopropylsilyl)ethynyl]pyrido[2,3-d]pyrimidin-7-one (200 mg, 0.371 mmol, 1.0 eq.), lithium chloride (1.57 mg, 37.0 μmol, 0.1 eq.), acetic anhydride (56.7 mg, 0.556 mmol, 1.5 eq.) and triethylamine (75.0 mg, 0.741 mmol, 2.0 eq.) in THF (2.0 mL) was stirred for 2 h at 70° C.

The mixture was allowed to cool to room temperature. The residue was purified by reverse phase flash column chromatography eluting with acetonitrile (30-100%) in water (0.1% NH₄HCO₃) to yield the title compound as a light yellow solid (120 mg, 56%).

### N-[5-ethynyl-2-[(2-methoxyphenyl)amino]-7-oxo-8-phenylpyrido[2,3-d]pyrimidin-6-yl]acetamide

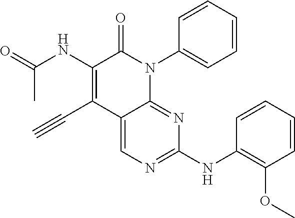 [SMILES: C#Cc1c(NC(C)=O)c(=O)n(-c2ccccc2)c2nc(Nc3ccccc3OC)ncc12]

General procedure 15 was applied to N-[2-[(2-methoxyphenyl)amino]-7-oxo-8-phenyl-5-[2-(triisopropylsilyl)ethynyl]pyrido[2,3-d]pyrimidin-6-yl]acetamide (120 mg, 0.206 mmol) and potassium fluoride (239 mg, 4.13 mmol) in THF (3.0 mL) and water (0.50 mL). The resulting solution was stirred for 12 h at room temperature. The residue was purified by reverse phase flash column chromatography eluting with acetonitrile (30-100%) in water (0.1% NH₄HCO₃) to yield the title compound as a light yellow solid (20.0 mg. 23%).

(ES, m/z): [M+H]⁺=426

¹H-NMR (300 MHz, CDCl₃) δ 8.99 (s, 1H), 8.11 (s, 1H), 7.66-7.57 (m, 4H), 7.48 (s, 2H), 7.31-7.26 (m, 2H), 6.91-6.79 (m, 2H), 6.50-6.46 (m, 1H), 4.00 (s, 2H), 3.86 (s, 3H), 2.25 (s, 3H).

### Example 144

### 8-methyl-2-(methylsulfanyl)-6-(phenylamino)-5-[2-(triisopropylsilyl)ethynyl]pyrido[2,3-d]pyrimidin-7-one

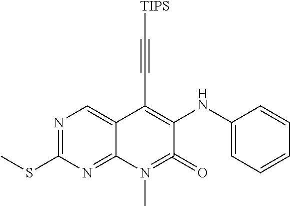

To a solution of 6-bromo-8-methyl-2-(methylsulfanyl)-5-[2-(triisopropylsilyl)ethynyl]pyrido[2,3-d]pyrimidin-7-one (1.50 g, 3.05 mmol, 1.00 eq.), aniline (440 mg, 4.58 mmol, 1.5 eq.), caesium carbonate (1.99 g, 6.11 mmol, 2.0 eq.) in dioxane (15 mL) was added XantPhos (180 mg, 0.306 mmol, 0.1 eq.) and palladium (II) acetate (70.0 mg, 0.306 mmol, 0.1 eq.). The resulting solution was stirred overnight at 100° C. under nitrogen then diluted with ethyl acetate (50 mL) and water (50 mL). The organic phase was separated and concentrated to dryness under reduced pressure. The residue was purified by flash column chromatography eluting with ethyl acetate (5%) in 40-60 petroleum ether to afford the title compound as a brown solid (1.00 g, 65%).

(ES, m/z): [M+H]⁺=479.2.

### 2-methanesulfonyl-8-methyl-6-(phenylamino)-5-[2-(triisopropylsilyl)ethynyl]pyrido[2,3-]pyrimidin-7-one

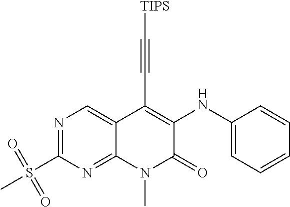

General procedure 10 was applied to 8-methyl-2-(methylsulfanyl)-6-(phenylamino)-5-[2-(triisopropylsilyl)ethynyl]pyrido[2,3-d]pyrimidin-7-one (1.00 g, 2.00 mmol) and m-CPBA (870 mg, 5.01 mmol) in dichloromethane (10 mL) to yield the title compound as a brown solid (700 mg, 66%).

(ES, m/z): [M+H]⁺=511.2.

### 2-[(4-{[2-(dimethylamino)ethyl](methyl)amino}phenyl)amino]-8-methyl-6-(phenylamino)-5-[2-(triisopropylsilyl)ethynyl]pyrido[2,3-d]pyrimidin-7-one

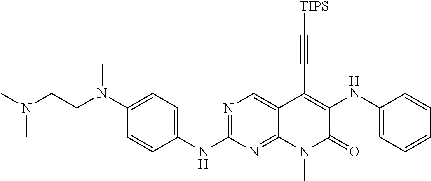

General procedure 13 was applied to 2-methanesulfonyl-8-methyl-6-(phenylamino)-5-[2-(triisopropylsilyl)ethynyl]pyrido[2,3-d]pyrimidin-7-one (700 mg, 1.32 mmol), trifluoroacetic acid (450 mg, 3.95 mmol) and N¹-[2-(dimethylamino)ethyl]-N¹-methylbenzene-1,4-diamine (381 mg, 1.97 mmol) in 2-butanol (6.72.0 mL). The solution mixture was stirred for 48 h at 100° C. The residue was purified by reverse phase flash column chromatography eluting with acetonitrile (30-60%) in water (0.1% formic acid) to yield the title compound as a brown solid (360 mg, 43%).

(ES, m/z): [M+H]⁺=624.4.

### 2-[(4-{[2-(dimethylamino)ethyl](methyl)amino}phenyl)amino]-5-ethynyl-8-methyl-6-(phenylamino)pyrido[2,3-d]pyrimidin-7-one

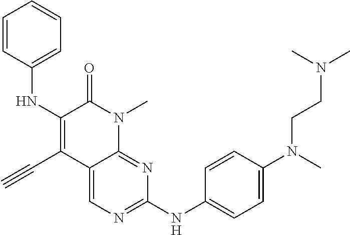 [SMILES: C#Cc1c(Nc2ccccc2)c(=O)n(C)c2nc(Nc3ccc(N(C)CCN(C)C)cc3)ncc12]

General procedure 15 was applied to 2-[(4-{[2-(dimethylamino)ethyl](methyl)amino}phenyl)amino]-8-methyl-6-(phenylamino)-5-[2-(triisopropylsilyl) ethynyl]pyrido[2,3-d]pyrimidin-7-one (360 mg, 0.565 mmol) and potassium fluoride (657 mg, 11.3 mmol) in THF (3.6 mL) and methanol (1.8 mL). The resulting mixture was stirred for 2 h at 80° C. The residue was purified by reverse phase flash column chromatography eluting with acetonitrile (30-60%) in water (0.1% formic acid) to yield the title compound as a brown-yellow solid (35.0 mg, 12%)

(ES, m/z): [M+H]⁺=468.2

¹H NMR (400 MHz, DMSO-d6) δ 9.68 (s, 1H), 8.69 (s, 1H), 8.00 (s, 1H), 7.60 (d, J=8.5 Hz, 2H), 7.19 (t, J=7.7 Hz, 2H), 6.94 (d, J=7.9 Hz, 2H), 6.84 (t, J=7.5 Hz, 1H), 6.72 (d, J=8.6 Hz, 2H), 4.92 (s, 1H), 3.65 (s, 3H), 3.42 (t, J=7.1 Hz, 2H), 2.88 (s, 3H), 2.49 (t, J=7.1 Hz, 2H), 2.28 (s, 6H).

### Example 145

### Ethyl 2-cyano-3-[2-(methylsulfanyl)-4-(phenylamino) pyrimidin-5-yl]-3-oxopropanoate

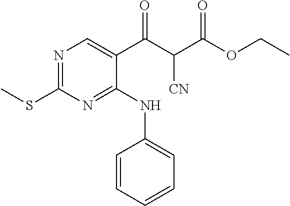 [SMILES: CCOC(=O)C(C#N)C(=O)c1cnc(SC)nc1Nc1ccccc1]

To a solution of ethyl cyanoacetate (2.34 g, 20.7 mmol, 1.5 eq.) in THF (50 mL) was added sodium hydride (660 mg, 60% in oil) at 0° C. The mixture was stirred for 15 min then 5-(1,2,3-benzotriazole-1-carbonyl)-2-(methylsulfanyl)-N-phenylpyrimidin-4-amine (5.00 g, 13.8 mmol, 1.0 eq.) in THF (30 mL) was added. The resulting solution was stirred for 1 h at 0° C. then quenched with ice-water (100 mL). The pH value of the solution was adjusted to 3 with 3M aqueous hydrochloric acid solution. The solids were collected by filtration and dried in a vacuum oven to yield the title compound as a yellow solid (5.00 g, 102% crude.

### 5-hydroxy-2-(methylsulfanyl)-7-oxo-8-phenylpyrido[2,3-d]pyrimidine-6-carbonitrile

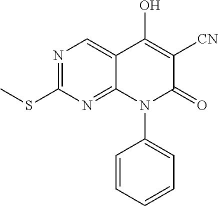 [SMILES: CSc1ncc2c(O)c(C#N)c(=O)n(-c3ccccc3)c2n1]

General procedure 6 was applied to ethyl 2-cyano-3-[2-(methylsulfanyl)-4-(phenylamino)pyrimidin-5-yl]-3-oxopropanoate (5.00 g, 14.0 mmol) in N,N-diisopropylethylamine (16 mL) and DBU (2.0 mL) to yield the title compound as a yellow solid (3.00 g, 69%).

### 5-chloro-2-(methylsulfanyl)-7-oxo-8-phenylpyrido[2,3-d]pyrimidine-6-carbonitrile

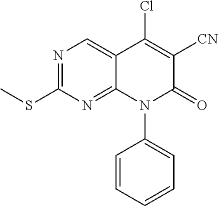 [SMILES: CSc1ncc2c(Cl)c(C#N)c(=O)n(-c3ccccc3)c2n1]

A solution of 5-hydroxy-2-(methylsulfanyl)-7-oxo-8-phenylpyrido [2,3-d]pyrimidine-6-carbonitrile (3.00 g, 9.67 mmol, 1.0 eq.), phosphorus oxytrichloride (4.45 g, 29.0 mmol, 3.0 eq.) in DMF (30 mL) was stirred for 2 h at 100° C. The reaction mixture was cooled to room temperature with a water/ice bath then diluted with water (100 mL). The solids were collected by filtration and dried in a vacuum oven to yield the title compound as a brown solid (3.20 g, 101%, crude).

### 2-(methylsulfanyl)-7-oxo-8-phenyl-5-[2-(triisopropylsilyl)ethynyl]pyrido[2,3-d]pyrimidine-6-carbonitrile

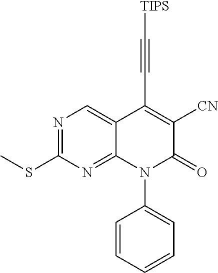

General procedure 9 was applied to 5-chloro-2-(methylsulfanyl)-7-oxo-8-phenylpyrido[2,3-d]pyrimidine-6-carbonitrile (3.00 g, 9.13 mmol), triisopropylsilylacetylene (3.33 g, 18.2 mmol), copper(I) iodide (174 mg, 0.912 mmol) and bis(triphenylphsophine)palladium(II) dichloride (640 mg, 0.912 mmol) in DMF (30 mL) and N,N-diisopropylethylamine (15 mL). The crude material was purified by flash column chromatography eluting with acetonitrile (40-100%) in water (0.1% NH₄HCO₃) to yield the title compound as a brown solid (1.00 g, 23%).

### 2-methanesulfonyl-7-oxo-8-phenyl-5-[2-(triisopropylsilyl)ethynyl]pyrido[2,3-d]pyrimidine-6-carbonitrile

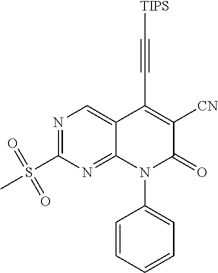

General procedure 10 was applied to 2-(methylsulfanyl)-7-oxo-8-phenyl-5-[2-(triisopropylsilyl) ethynyl]pyrido[2,3-d]pyrimidine-6-carbonitrile (1.00 g, 2.11 mmol), m-CPBA (1.09 g, 6.32 mmol), dichloromethane (10 mL). The crude material was purified by reverse phase flash column chromatography eluting with acetonitrile (40-50%) in water (0.1% NH₄HCO₃) to yield the title compound as a yellow solid (500 mg, 47%).

### 2-[(2-methoxyphenyl)amino]-7-oxo-8-phenyl-5-[2-(triisopropylsilyl)ethynyl]pyrido[2,3-d]pyrimidine-6-carbonitrile

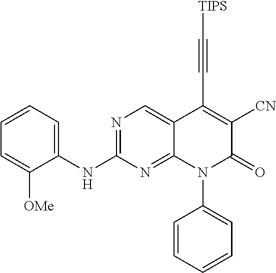

General procedure 13 was applied to 2-methanesulfonyl-7-oxo-8-phenyl-5-[2-(triisopropylsilyl) ethynyl]pyrido[2,3-d]pyrimidine-6-carbonitrile (500 mg, 0.987 mmol), o-anisidine (121 mg, 0.987 mmol), trifluoroacetic acid (112 mg, 0.987 mmol) and 2-butanol (5.0 mL). The reaction mixture was stirred at 110° C. for 1 h. The reaction mixture was cooled to room temperature and applied onto a reverse phase column directly with eluting with acetonitrile (40-100%) in water (0.1% NH₄HCO₃) to yield the title compound as a yellow solid (300 mg, 55%).

### 5-ethynyl-2-[(2-methoxyphenyl)amino]-7-oxo-8-phenylpyrido[2,3-d]pyrimidine-6-carbonitrile

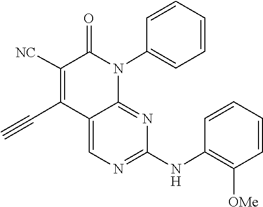 [SMILES: C#Cc1c(C#N)c(=O)n(-c2ccccc2)c2nc(Nc3ccccc3OC)ncc12]

General procedure 15 was applied to 2-[(2-methoxyphenyl)amino]-7-oxo-8-phenyl-5-[2-(triisopropylsilyl) ethynyl]pyrido[2,3-d]pyrimidine-6-carbonitrile (200 mg, 0.364 mmol), trifluoroacetic acid (41.5 mg, 0.364 mmol) and potassium fluoride (211 mg, 3.64 mmol) in THF (4.0 mL), water (1.0 mL). The resulting solution was stirred for 2 h at 50° C. The crude product was purified by Prep-HPLC eluting with acetonitrile (10-40%) in water (0.05% formic acid) to yield the title compound as an orange solid (19.0 mg, 13%).

(ES, m/z): [M+H]⁺=394

¹H-NMR (300 MHz, CDCl₃) δ 8.97 (s, 1H), 8.37 (s, 1H), 7.65-7.63 (m, 3H), 7.42 (d, J=8.3 Hz, 1H), 7.32-7.30 (m, 2H), 6.97 (t, J=7.8 Hz, 1H), 6.85 (d, J=8.0 Hz, 1H), 6.49 (t, J=7.5 Hz, 1H), 4.23 (s, 1H), 3.90 (s, 3H).

### Example 146

### N,N-dimethyl-2-(methylsulfanyl)-7-oxo-8-phenyl-5-[2-(triisopropylsilyl)ethynyl]pyrido[2,3-d]pyrimidine-6-carboxamide

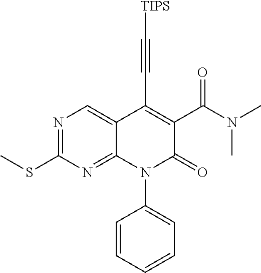

A solution of 2-(methylsulfanyl)-7-oxo-8-phenyl-5-[2-(triisopropylsilyl)ethynyl]pyrido[2,3-d]pyrimidine-6-carboxylic acid (300 mg, 0.608 mmol, 1.0 eq.), dimethylamine (32.9 mg, 0.729 mmol, 1.2 eq.), N,N-diisopropylethylamine (157 mg, 1.22 mmol, 2.0 eq.) and HATU (277 mg, 0.729 mmol, 1.2 eq.) in DMF (3.0 mL) was stirred for 2 h at room temperature. The resulting solution was diluted with water (20 mL) and extracted with ethyl acetate (3×20 mL). The combined organic layers were washed with brine (3×20 mL) and concentrated under reduced pressure. The crude material was purified by flash column chromatography eluting with ethyl acetate (50%) in 40-60 petroleum ether to yield the title compound as a yellow solid (280 mg, 88%).

### 2-methanesulfonyl-N,N-dimethyl-7-oxo-8-phenyl-5-[2-(triisopropylsilyl)ethynyl]pyrido[2,3-d]pyrimidine-6-carboxamide

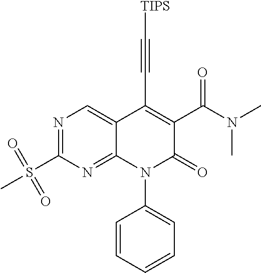

General procedure 10 was applied to N,N-dimethyl-2-(methylsulfanyl)-7-oxo-8-phenyl-5-[2-(triisopropylsilyl)ethynyl]pyrido[2,3-d]pyrimidine-6-carboxamide (280 mg, 0.538 mmol) and m-CPBA (278 mg, 1.61 mmol) in dichloromethane (5.00 mL). The crude material was purified by reverse phase flash column chromatography eluting with acetonitrile (35-100%) in water (0.1% NH₄HCO₃) to yield the title compound as a light yellow solid (200 mg, 67%).

### 2-[(2-methoxyphenyl)amino]-N,N-dimethyl-7-oxo-8-phenyl-5-[2-(triisopropylsilyl)ethynyl]pyrido[2,3-d]pyrimidine-6-carboxamide

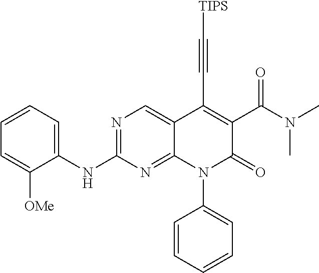

General procedure 13 was applied to 2-methanesulfonyl-N,N-dimethyl-7-oxo-8-phenyl-5-[2-(triisopropylsilyl) ethynyl]pyrido[2,3-d]pyrimidine-6-carboxamide (200 mg, 0.362 mmol), o-anisidine (49.0 mg, 0.398 mmol), trifluoroacetic acid (45.4 mg, 0.398 mmol) in 2-butanol (2.0 mL). The resulting solution was stirred for 1 h at 110° C. before being purified by reverse phase flash column chromatography, eluting with acetonitrile (30-100%) in water (0.1% NH₄HCO₃) to yield the title compound as a yellow solid (150 mg, 70%).

### 5-ethynyl-2-[(2-methoxyphenyl)amino]-N,N-dimethyl-7-oxo-8-phenylpyrido[2,3-d]pyrimidine-6-carboxamide

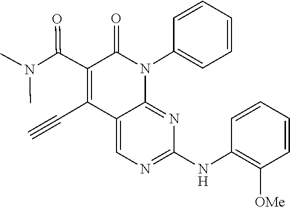 [SMILES: C#Cc1c(C(=O)N(C)C)c(=O)n(-c2ccccc2)c2nc(Nc3ccccc3OC)ncc12]

General procedure 15 was applied to 2-[(2-methoxyphenyl)amino]-N,N-dimethyl-7-oxo-8-phenyl-5-[2-(triisopropylsilyl)ethynyl]pyrido[2,3-d]pyrimidine-6-carboxamide (150 mg, 0.252 mmol) and potassium fluoride (146 mg, 2.52 mmol) in THF (4.0 mL) and water (1.0 mL). The resulting solution was stirred for 12 h at room temperature. The crude product was purified by Prep-HPLC eluting with acetonitrile (40-70%) in water (0.1% NH₄HCO₃) to yield the title compound as a light yellow solid (33.0 mg, 30%).

(ES, m/z): [M+H]⁺=440

¹H NMR (300 MHz, CDCl₃) δ 8.93 (s, 1H), 8.33 (s, 1H), 7.64-7.50 (m, 4H), 7.32 (s, 2H), 6.99-6.88 (m, 1H), 6.85-8.82 (m, 1H), 6.51 (s, 1H), 3.88 (d, J=2.5 Hz, 4H), 3.18 (s, 3H), 3.08 (s, 3H).

### Example 147

### 6-(dimethylamino)-2-(methylsulfanyl)-8-phenyl-5-[2-(triisopropylsilyl)ethynyl]pyrido[2,3-d]pyrimidin-7-one

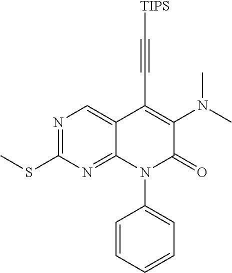

To a solution of 6-amino-2-(methylsulfanyl)-8-phenyl-5-[2-(triisopropylsilyl)ethynyl]pyrido[2,3-d]pyrimidin-7-one (500 mg, 1.07 mmol, 1.0 eq.) in THF (5.0 mL) was added sodium hydride (64.5 mg, 2.69 mmol, 2.5 eq.) at 0° C. The mixture was stirred for 15 min then methyl iodide (305 mg, 2.15 mmol, 2.0 eq.) was added and the mixture was allowed to warm to room temperature and stirred for 2 h. The reaction mixture was quenched with water and extracted with dichloromethane (3×30 mL). The combined organic layers were washed with brine (2×20 mL), dried (Na₂SO₄) and concentrated under reduced pressure. The residue was purified by reverse phase flash column chromatography eluting with acetonitrile and water (0.1% NH₄HCO₃) to yield the title compound as a yellow solid (370 mg, 70%).

### 6-(dimethylamino)-2-methanesulfonyl-8-phenyl-5-[2-(triisopropylsilyl)ethynyl]pyrido[2,3-d]pyrimidin-7-one

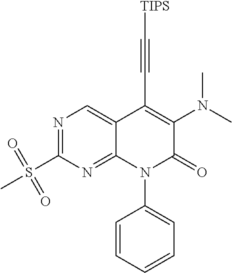

General procedure 10 was applied to 6-(dimethylamino)-2-(methylsulfanyl)-8-phenyl-5-[2-(triisopropylsilyl) ethynyl]pyrido[2,3-d]pyrimidin-7-one (370 mg, 0.751 mmol) and m-CPBA (259 mg, 1.50 mmol) in dichloromethane (5.0 mL). The residue was purified by reverse phase flash column chromatography eluting with acetonitrile in water (0.1% NH₄HCO₃) to yield the title compound as a light yellow solid (195 mg, 49%).

### 6-(dimethylamino)-2-[(2-methoxyphenyl)amino]-8-phenyl-5-[2-(triisopropylsilyl)ethynyl]pyrido[2,3-d]pyrimidin-7-one

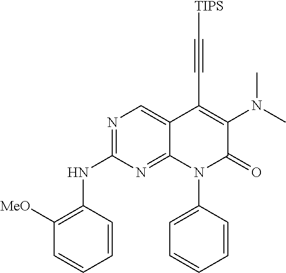

General procedure 13 was applied to 6-(dimethylamino)-2-methanesulfonyl-8-phenyl-5-[2-(triisopropylsilyl) ethynyl]pyrido[2,3-d]pyrimidin-7-one (195 mg, 0.372 mmol), o-anisidine (50.3 mg, 0.409 mmol) and trifluoroacetic acid (46.6 mg, 0.409 mmol) in 2-butanol (2.0 mL). The resulting solution was stirred for 2 h at 100° C. The crude product was purified by Prep-HPLC eluting with acetonitrile (30-100%) in water (0.1% NH₄HCO₃) to yield the title compound as a light yellow solid (100 mg, 47%).

### 6-(dimethylamino)-5-ethynyl-2-[(2-methoxyphenyl)amino]-8-phenylpyrido[2,3-d]pyrimidin-7-one

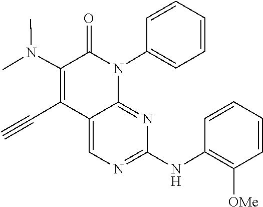 [SMILES: C#Cc1c(N(C)C)c(=O)n(-c2ccccc2)c2nc(Nc3ccccc3OC)ncc12]

General procedure 15 was applied to 6-(dimethylamino)-2-[(2-methoxyphenyl)amino]-8-phenyl-5-[2-(triisopropylsilyl)ethynyl]pyrido[2,3-d]pyrimidin-7-one (100 mg, 0.176 mmol) potassium fluoride (205 mg, 3.52 mmol) in THF (5.0 mL) and water (1.0 mL). The resulting solution was stirred for 3 days at room temperature. The residue was purified by reverse phase flash column chromatography eluting with acetonitrile (35-100%) in water (0.1% NH₄HCO₃) to yield the title compound as a yellow solid (20.0 mg, 28%).

(ES, m/z): [M+H]⁺=412

¹H-NMR (300 MHz, CDCl₃) δ 8.89 (s, 1H), 7.97 (s, 1H), 7.70-7.52 (m, 4H), 7.37-7.27 (m, 2H), 6.92-6.77 (m, 2H), 6.50 (t, J=7.7 Hz, 1H), 3.94 (s, 1H), 3.88 (s, 3H), 3.19 (s, 6H).

### Example 148

### 6-cyclopropyl-8-methyl-2-(methylsulfanyl)-5-[2-(triisopropylsilyl)ethynyl]pyrido[2,3-d]pyrimidin-7-one

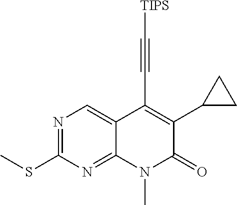

General procedure 17 was applied to 6-bromo-8-methyl-2-(methylsulfanyl)-5-[2-(triisopropylsilyl)ethynyl]pyrido[2,3-d]pyrimidin-7-one (2.00 g, 4.28 mmol), cyclopropylboronic acid (740 mg, 8.57 mmol), potassium carbonate (1.19 g, 8.57 mmol) and (1,1′-Bis(diphenylphosphino)ferrocene)palladium(II) dichloride (310 mg, 0.429 mmol) in dioxane (16 mL) and water (4 mL). The residue was purified by flash column chromatography eluting with ethyl acetate (0-25%) in 40-60 petroleum ether to yield the title compound as a yellow solid (1.17 g, 64%).

(ES, m/z): [M+H]⁺=428.2

### 6-cyclopropyl-2-methanesulfonyl-8-methyl-5-[2-(triisopropylsilyl)ethynyl]pyrido[2,3-d]pyrimidin-7-one

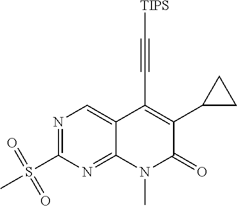

General procedure 10 was applied to 6-cyclopropyl-8-methyl-2-(methylsulfanyl)-5-[2-(triisopropylsilyl)ethynyl]pyrido[2,3-d]pyrimidin-7-one (1.00 g, 2.34 mmol) and m-CPBA (890 g, 5.14 mmol) in dichloromethane (10 mL) to yield the title compound as a yellow solid (970 mg, 90%).

(ES, m/z): [M+H]⁺=460.2

### 6-cyclopropyl-8-methyl-2-{[4-(4-methylpiperazin-1-yl)phenyl]amino}-5-[2-(triisopropylsilyl)ethynyl]pyrido[2,3-d]pyrimidin-7-one

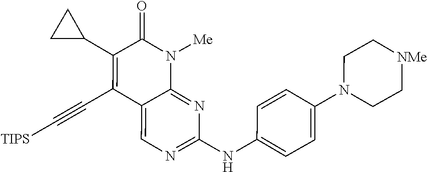

General procedure 13 was applied to 6-cyclopropyl-2-methanesulfonyl-8-methyl-5-[2-(triisopropylsilyl)ethynyl]pyrido[2,3-d]pyrimidin-7-one (900 mg, 1.96 mmol), 4-(4-methylpiperazin-1-yl)aniline (449 mg, 2.35 mmol) and trifluoroacetic acid (446 mg, 3.92 mmol) in 2-butanol (10 mL). The resulting solution was stirred for 3 h at 100° C. before being purified by reverse phase flash column chromatography, eluting with acetonitrile (70-100%) in water (0.1% trifluoroacetic acid). The resulting mixture was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (500 mg, 45%).

(ES, m/z): [M+H]⁺=571.4

### 6-cyclopropyl-5-ethynyl-8-methyl-2-{[4-(4-methylpiperazin-1-yl)phenyl]amino}pyrido[2,3-d]pyrimidin-7-one

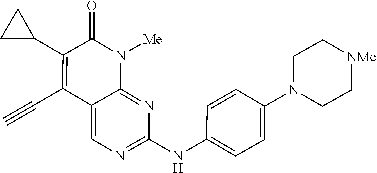 [SMILES: C#Cc1c(C2CC2)c(=O)n(C)c2nc(Nc3ccc(N4CCN(C)CC4)cc3)ncc12]

General procedure 15 was applied to 6-cyclopropyl-8-methyl-2-{[4-(4-methylpiperazin-1-yl)phenyl]amino}-5-[2-(triisopropylsilyl)ethynyl]pyrido[2,3-d]pyrimidin-7-one (300 mg, 0.526 mmol) with potassium fluoride in THF (4 mL). The resulting solution was stirred for 2 h at 50° C. before being purified by reverse phase flash column chromatography, eluting with acetonitrile (20-100%) in water (0.1% formic acid). The desired fractions were collected and concentrated under reduced pressure to remove most of the acetonitrile then lyophilized to yield the title compound as a dark yellow solid (150 mg, 69%).

(ES, m/z): [M+H]⁺=415.3

¹H NMR (300 MHz, DMSO-d6) δ 9.78 (s, 1H), 8.76 (s, 1H), 7.68 (s, 2H), 7.03 (s, 2H), 5.20 (s, 1H), 3.76 (s, 2H), 3.54 (s, 5H), 3.15 (s, 2H), 2.87 (s, 5H), 2.20 (s, 1H), 1.42 (s, 2H), 0.87 (s, 2H).

### Example 149

### 6,8-dimethyl-2-(methylsulfanyl)-5-[2-(triisopropylsilyl)ethynyl]pyrido[2,3-d]pyrimidin-7-one

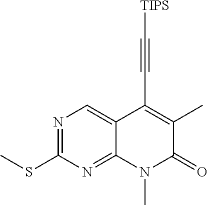

General procedure 17 was applied to 6-bromo-8-methyl-2-(methylsulfanyl)-5-[2-(triisopropylsilyl)ethynyl]pyrido[2,3-d]pyrimidin-7-one (2.00 g, 4.29 mmol), methylboronic acid (510 mg, 8.57 mmol), potassium carbonate (1.19 g, 8.57 mmol) and (1,1′-Bis(diphenylphosphino)ferrocene)palladium(II) dichloride (310 mg, 0.429 mmol) in dioxane (16 mL) and water (4.0 mL). The crude material was purified by flash column chromatography eluting with ethyl acetate (0-75%) in 40-60 petroleum ether to yield the title compound as a yellow solid (700 mg, 41%).

(ES, m/z): [M+H]⁺=402.2

### 2-methanesulfonyl-6,8-dimethyl-5-[2-(triisopropylsilyl)ethynyl]pyrido[2,3-d]pyrimidin-7-one

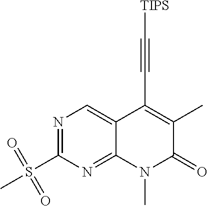

General procedure 10 was applied to 6,8-dimethyl-2-(methylsulfanyl)-5-[2-(triisopropylsilyl)ethynyl]pyrido[2,3-d]pyrimidin-7-one (700 mg, 1.74 mmol) and m-CPBA (661 mg, 3.83 mmol) in dichloromethane to yield the title compound as a yellow solid (730 mg, 97%).

(ES, m/z): [M+H]⁺=434.2

### 6,8-dimethyl-2-{[4-(4-methylpiperazin-1-yl)phenyl]amino}-5-[2-(triisopropylsilyl)ethynyl]pyrido[2,3-d]pyrimidin-7-one

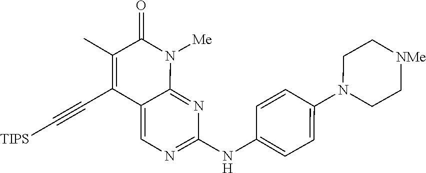

General procedure 13 was applied to 2-methanesulfonyl-6,8-dimethyl-5-[2-(triisopropylsilyl)ethynyl]pyrido[2,3-d]pyrimidin-7-one (700 mg, 1.61 mmol), trifluoroacetic acid (368 mg, 3.23 mmol), 4-(4-methylpiperazin-1-yl)aniline (370 mg, 1.94 mmol) and 2-butanol (5.0 mL). The resulting solution was stirred for 3 h at 100° C. before being purified by reverse phase flash column chromatography, eluting with acetonitrile (70-100%) in water (0.1% trifluoroacetic acid). The mixture was acidified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (400 mg, 45%).

(ES, m/z): [M+H]⁺=545.3

### 5-ethynyl-6,8-dimethyl-2-{[4-(4-methylpiperazin-1-yl)phenyl]amino}pyrido[2,3-d]pyrimidin-7-one

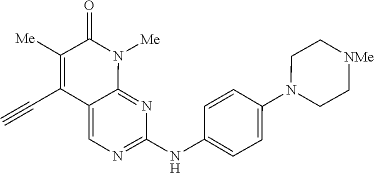 [SMILES: C#Cc1c(C)c(=O)n(C)c2nc(Nc3ccc(N4CCN(C)CC4)cc3)ncc12]

General procedure 15 was applied to 6,8-dimethyl-2-{[4-(4-methylpiperazin-1-yl)phenyl]amino}-5-[2-(triisopropylsilyl)ethynyl]pyrido[2,3-d]pyrimidin-7-one (400 mg, 0.734 mmol) and potassium fluoride (426 mg, 7.34 mmol) in THF (4.0 mL). The resulting solution was stirred for 2 h at 50° C. before being purified by reverse phase flash column chromatography, eluting with acetonitrile (20-100%) in water (0.1% formic acid). The desired fractions were collected and concentrated under reduced pressure to remove most of the acetonitrile then lyophilized to dryness to yield the title compound as a dark yellow solid (140 mg, 45%).

(ES, m/z): [M+H]⁺=388.9

¹H NMR (300 MHz, CDCl₃) δ 8.82 (s, 1H), 7.56 (s, 3H), 6.95 (s, 2H), 3.82 (s, 1H), 3.72 (s, 3H), 3.31 (s, 4H), 2.95 (s, 4H), 2.57 (s, 3H), 2.37 (s, 3H).

### Example 150

### 8-methyl-2-(methylsulfanyl)-6-(thiophen-2-yl)-5-[2-(triisopropylsilyl) ethynyl]pyrido[2,3-]pyrimidin-7-one

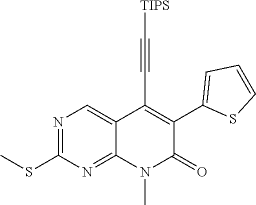

General procedure 17 was applied to 6-bromo-8-methyl-2-(methylsulfanyl)-5-[2-(triisopropylsilyl)ethynyl]pyrido[2,3-d]pyrimidin-7-one (2.00 g, 4.29 mmol), thiophen-2-ylboronic acid (1.10 g, 8.57 mmol), potassium carbonate (1.19 g, 8.57 mmol) and (1,1′-Bis(diphenylphosphino)ferrocene)palladium(II) dichloride (310 mg, 0.429 mmol) in dioxane (16 mL) and water (4.0 mL). The crude material was purified by flash column chromatography eluting with ethyl acetate (0-75%) in 40-60 petroleum ether to yield the title compound as a white solid (739 mg, 37%).

(ES, m/z): [M+H]⁺=470.2

### 2-methanesulfonyl-8-methyl-6-(thiophen-2-yl)-5-[2-(triisopropylsilyl)ethynyl]pyrido[2,3-d]pyrimidin-7-one

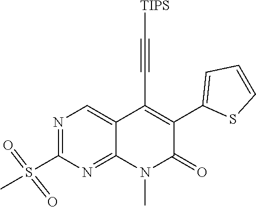

General procedure 10 was applied to 8-methyl-2-(methylsulfanyl)-6-(thiophen-2-yl)-5-[2-(triisopropylsilyl)ethynyl]pyrido[2,3-d]pyrimidin-7-one (700 mg, 1.49 mmol) and m-CPBA (566 mg, 3.28 mmol) in dichloromethane to yield the title compound as a yellow solid (700 mg, 94%).

(ES, m/z): [M+H]⁺=502.2

### 8-methyl-2-{[4-(4-methylpiperazin-1-yl)phenyl]amino}-6-(thiophen-2-yl)-5-[2-(triisopropylsilyl)ethynyl]pyrido[2,3-d]pyrimidin-7-one

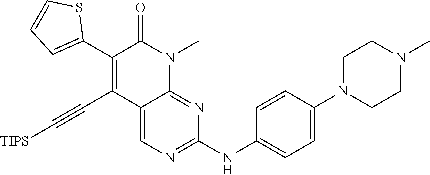

General procedure 13 was applied to 2-methanesulfonyl-8-methyl-6-(thiophen-2-yl)-5-[2-(triisopropylsilyl) ethynyl]pyrido[2,3-d]pyrimidin-7-one (700 mg, 1.39 mmol), trifluoroacetic acid (318 mg, 2.79 mmol), 4-(4-methylpiperazin-1-yl)aniline (320 mg, 1.67 mmol) and 2-butanol (5.0 mL). The resulting solution was stirred for 3 h at 100° C. before being purified by reverse phase flash column chromatography, eluting with acetonitrile (70-100%) in water (0.1% trifluoroacetic acid). The mixture was basified to pH 8 with saturated aqueous sodium hydrogen carbonate solution and extracted with ethyl acetate (3×200 mL). The combined organic layers were washed with brine (2×200 mL), dried (Na₂SO₄) and concentrated under reduced pressure to yield the title compound as a yellow solid (400 mg, 47%).

(ES, m/z): [M+H]⁺=613.3

### 5-ethynyl-8-methyl-2-{[4-(4-methylpiperazin-1-yl)phenyl]amino}-6-(thiophen-2-yl)pyrido[2,3-d]pyrimidin-7-one

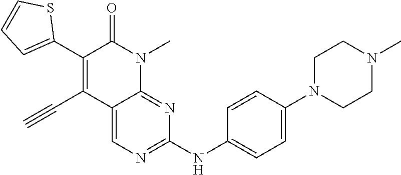 [SMILES: C#Cc1c(-c2cccs2)c(=O)n(C)c2nc(Nc3ccc(N4CCN(C)CC4)cc3)ncc12]

General procedure 15 was applied to 8-methyl-2-{[4-(4-methylpiperazin-1-yl)phenyl]amino}-6-(thiophen-2-yl)-5-[2-(triisopropylsilyl)ethynyl]pyrido[2,3-d]pyrimidin-7-one (200 mg, 0.326 mmol), potassium fluoride (189 mg, 3.26 mmol) in THF (3.0 mL). The resulting solution was stirred for 2 h at 50° C. before being purified by reverse phase flash column chromatography, eluting with acetonitrile (20-100%) in water (0.1% formic acid). The desired fractions were collected and concentrated under reduced pressure to remove most of the acetonitrile then lyophilized to dryness to yield the title compound as an orange solid (65.0 mg, 42%).

(ES, m/z): [M+H]⁺=457.2

¹H NMR (400 MHz, DMSO-d₆) δ 10.11 (s, 1H), 8.91 (s, 1H), 8.05 (s, 1H), 7.69 (d, J=17.7 Hz, 3H), 7.17 (s, 1H), 6.96 (s, 2H), 5.28 (s, 1H), 3.65 (s, 3H), 3.17 (s, 4H), 2.68 (s, 4H), 2.39 (s, 3H).

### Example 151

### 6-Methyl-2-(methylsulfanyl)-8-phenyl-5-[2-(triisopropylsilyl)ethynyl]pyrido[2,3-]pyrimidin-7-one

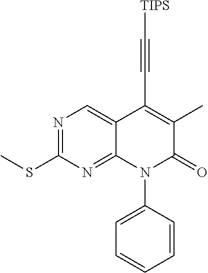

General procedure 17 was applied to 6-bromo-2-(methylsulfanyl)-8-phenyl-5-[2-(triisopropylsilyl) ethynyl]pyrido[2,3-d]pyrimidin-7-one (750 mg, 1.42 mmol), methylboronic acid (170 mg, 2.84 mmol), (1,1′-bis(diphenylphosphino)ferrocene)palladium(II) dichloride (104 mg, 0.142 mmol) and potassium carbonate (392 mg, 2.84 mmol) in dioxane (7.5 mL) and water (1.5 mL). The resulting solution was stirred for 3 h at 75° C. before being purified by reverse phase flash column chromatography, eluting with acetonitrile (20-100%) in water (0.1% trifluoroacetic acid) to yield the title compound as an off-white solid (450 mg, 68%).

### 2-methanesulfonyl-6-methyl-8-phenyl-5-[2-(triisopropylsilyl)ethynyl]pyrido[2,3-d]pyrimidin-7-one

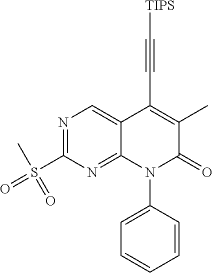

General procedure 10 was applied to 6-methyl-2-(methylsulfanyl)-8-phenyl-5-[2-(triisopropylsilyl) ethynyl]pyrido[2,3-d]pyrimidin-7-one (450 mg, 0.970 mmol) and m-CPBA (418 mg, 2.42 mmol) in dichloromethane (5.0 mL) to yield the title compound as a yellow solid (450 mg, 94%).

### 6-methyl-2-{[4-(4-methylpiperazin-1-yl)phenyl]amino}-8-phenyl-5-[2-(triisopropylsilyl)ethynyl]pyrido[2,3-d]pyrimidin-7-one

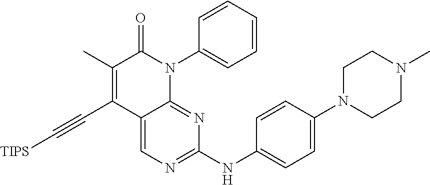

General procedure 13 was applied to 2-methanesulfonyl-6-methyl-8-phenyl-5-[2-(triisopropylsilyl)ethynyl]pyrido[2,3-d]pyrimidin-7-one (450 mg, 0.908 mmol), trifluoroacetic acid (207 mg, 1.82 mmol) and 4-(4-methylpiperazin-1-yl)aniline (208 mg, 1.09 mmol) in 2-butanol (5.0 mL). The resulting solution was stirred for 3 h at 100° C. before being purified by reverse phase flash column chromatography, eluting with acetonitrile (70-100%) in water (0.1% trifluoroacetic acid). The mixture was basified to pH 8 with saturated aqueous sodium hydrogen carbonate and extracted with ethyl acetate (3×200 mL). The combined organic layers were washed with brine (2×200 mL), dried (Na₂SO₄) and concentrated under reduced pressure to yield the title compound as a yellow solid (300 mg, 54%).

### 5-Ethynyl-6-methyl-2-{[4-(4-methylpiperazin-1-yl)phenyl]amino}-8-phenylpyrido[2,3-d]pyrimidin-7-one

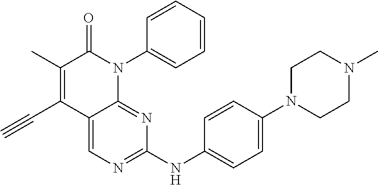 [SMILES: C#Cc1c(C)c(=O)n(-c2ccccc2)c2nc(Nc3ccc(N4CCN(C)CC4)cc3)ncc12]

General procedure 15 was applied to 6-methyl-2-{[4-(4-methylpiperazin-1-yl)phenyl]amino}-8-phenyl-5-[2-(triisopropylsilyl)ethynyl]pyrido[2,3-d]pyrimidin-7-one (300 mg, 0.495 mmol) and potassium fluoride (287 mg, 4.95 mmol) in THF (3.0 mL), DMF (3.0 mL) and water (0.50 mL). The resulting solution was stirred for 2 h at 70° C. before being purified by reverse phase flash column chromatography, eluting with acetonitrile (20-100%) in water (0.1% trifluoroacetic acid). The desired fractions were collected and concentrated under reduced pressure to remove most of the acetonitrile then lyophilized to yield the title compound as a yellow solid (167 mg, 75%).

(ES, m/z): [M+H]⁺=451

¹H-NMR (300 MHz, DMSO-d₆) δ 9.88 (s, 1H), 8.78 (s, 1H), 7.63 (d, J=5.4 Hz, 3H), 7.35 (d, J=2.4 Hz, 2H), 7.11 (d, J=6 Hz, 2H), 6.92 (d, J=9 Hz, 2H), 5.27 (s, 1H), 2.99-2.98 (m, 4H), 2.51-2.50 (m, 4H), 2.27 (s, 3H), 2.22 (s, 3H).

### Example 152

### 5-ethenyl-2-[(2-methoxyphenyl)amino]-8-phenylpyrido[2,3-d]pyrimidin-7-one

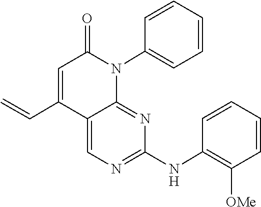 [SMILES: C=Cc1cc(=O)n(-c2ccccc2)c2nc(Nc3ccccc3OC)ncc12]

A solution of 5-ethynyl-2-[(2-methoxyphenyl)amino]-8-phenylpyrido[2,3-d]pyrimidin-7-one (200 mg, 1.0 eq.), Lindlar catalyst (30 mg, 15 wt %) in THF and methanol (v/v, 2:1, 15 mL). The vial was sealed and then evacuated and backfilled with hydrogen (3 cycles). After stirring the mixture at room temperature for 24 h, the palladium on carbon was filtered out and washed with THF (3×10 mL). The resulting filtrate was concentrated under reduced pressure and purified by reverse phase flash column chromatography. The desired fractions were collected and concentrated under reduced pressure to remove most of the acetonitrile then lyophilized to yield the title compound as light yellow solid (102 mg 51%).

(ES, m/z): [M+H]⁺=371

¹H NMR (300 MHz, Methanol-d₄) δ 8.96 (s, 1H), 7.71-7.57 (m, 3H), 7.48 (d, J=8.2 Hz, 1H), 7.39-7.18 (m, 3H), 6.96-6.83 (m, 2H), 6.65 (d, J=0.8 Hz, 1H), 6.46 (d, J=8.2 Hz, 1H), 6.12 (dd, J=17.2, 1.0 Hz, 1H), 5.79 (dd, J=11.0, 1.0 Hz, 1H), 3.89 (s, 3H).

### Example 153

### Ethyl 2,4-bis((2-methoxyphenyl)amino)pyrimidine-5-carboxylate

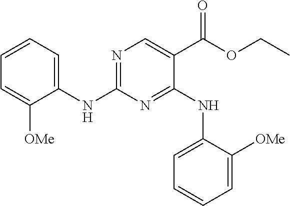 [SMILES: CCOC(=O)c1cnc(Nc2ccccc2OC)nc1Nc1ccccc1OC]

To a solution of ethyl 2,4-dichloropyrimidine-5-carboxylate (3.00 g, 13.6 mmol, 1.0 eq.) in a mixture of ethanol and THF (196 mL: 75 mL) was added o-anisidine (3.10 mL, 27.5 mmol, 2.0 eq.) and 1M aqueous hydrochloric acid (1.36 mL). The mixture was heated at 80° C. for 4 h, then cooled to room temperature. The product was collected by vacuum filtration washing with ethanol to yield the title compound as a white powder (4.39 g, 11.1 mmol, 82%).

(ES, m/z): [M+H]⁺=395.3

¹H NMR (500 MHz, CDCl₃) δ 1.35 (3H, t, J=7.1 Hz), 3.83 (3H, s), 3.87 (3H, s), 4.35 (2H, q, J=7.1 Hz), 6.71-6.85 (2H, m), 6.84-6.95 (2H, m), 7.14 (2H, dtd, J=9.6, 7.9, 1.6 Hz), 7.76 (1H, dd, J=8.0, 1.6 Hz), 8.00 (1H, dd, J=8.1, 1.6 Hz), 8.51 (1H, s), 15.44 (1H, s), NH not visualised.

### 2,4-bis((2-Methoxyphenyl)amino)pyrimidine-5-carboxylic acid

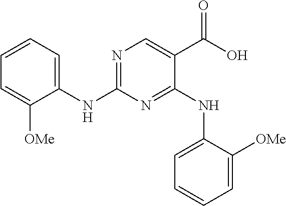 [SMILES: COc1ccccc1Nc1ncc(C(=O)O)c(Nc2ccccc2OC)n1]

1M aqueous sodium hydroxide solution (70.9 mL, 70.9 mmol, 7.0 eq.) was slowly added into a solution of ethyl 2,4-bis((2-methoxyphenyl)amino)pyrimidine-5-carboxylate (4.30 g, 10.9 mmol, 1.0 eq.) in THF (71 mL), then the mixture was stirred at 80° C. overnight. The solution was acidified to PH 4-5 with 4M aqueous hydrochloric acid after the solvent was partly removed under reduced pressure. The resulting solid was collected by vacuum filtration washing with water and dried in a vacuum oven (40° C.) to give the title compound as an off-white solid (2.50 g, 6.82 mmol, 63%).

(ES, m/z): [M+H]⁺=367.2

¹H NMR (500 MHz, DMSO-d₆) δ_{H} 3.79 (3H, s), 3.86 (3H, s), 6.73-6.80 (1H, m), 6.95 (1H, td, J=7.7, 1.4 Hz), 7.01-7.08 (2H, m), 7.10 (1H, dd, J=8.3, 1.4 Hz), 7.19 (1H, td, J=7.8, 1.7 Hz), 7.68 (1H, d, J=7.8 Hz), 8.26-8.30 (1H, m), 8.67 (1H, s), 8.95 (1H, s), 10.91 (1H, s), 13.01 (1H, s)

### 2,4-bis((2-Methoxyphenyl)amino)pyrimidine-5-carbonyl chloride

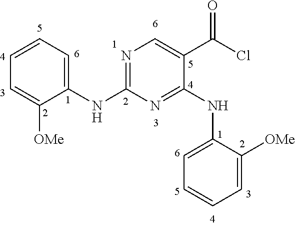 [SMILES: COc1ccccc1Nc1ncc(C(=O)Cl)c(Nc2ccccc2OC)n1]

To a solution of 2,4-bis((2-methoxyphenyl)amino)pyrimidine-5-carboxylic acid (2.50 g, 6.82 mmol, 1.0 eq.) in dichloromethane (68 mL) was added oxalyl chloride (6.8 mL) and DMF (6.8 mL). The resulting mixture was stirred at room temperature overnight. The solvent was evaporated to yield the title compound as a white solid (2.60 g, 6.76 mmol, 99%), which was used without further purification.

### 5-Hydroxy-8-(2-methoxyphenyl)-2-((2-methoxyphenyl)amino)pyrido[2,3-d]pyrimidin-7(8H)-one

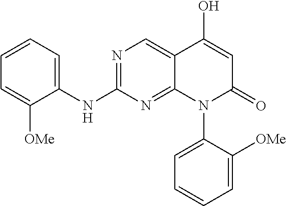 [SMILES: COc1ccccc1Nc1ncc2c(O)cc(=O)n(-c3ccccc3OC)c2n1]

General Procedure 5 was applied to 2,4-bis((2-methoxyphenyl)amino)pyrimidine-5-carbonyl chloride (2.60 g, 6.76 mmol) with 1M LiHMDS in THF (30.4 mL, 30.4 mmol), ethyl acetate (1.32 mL, 13.5 mmol) and THF (200 mL). The crude product was purified by flash column chromatography eluting with ethyl acetate (0-100%) in 40-60 petroleum ether to yield a brown solid (193 mg). General procedure 6 was applied to the resulting solid (193 mg, 0.440 mmol) with N,N-diisopropylethylamine (0.54 mL) and DBU (0.066 mL) to obtain the title compound as a beige solid (135 mg, 0.346 mmol, 5%).

(ES, m/z): [M+H]⁺=391.2

¹H NMR (500 MHz, DMSO-d₆) b 3.64 (3H, d, J=4.7 Hz), 3.77-3.88 (3H, s), 5.72 (1H, s), 6.45-6.53 (2H, m), 6.87-6.94 (1H, m), 6.95 (1H, dd, J=8.2, 1.5 Hz), 7.10 (1H, td, J=7.5, 1.3 Hz), 7.24 (1H, ddd, J=7.7, 4.9, 1.5 Hz), 7.43 (1H, d, J=8.1 Hz), 7.53 (1H, dd, J=7.8, 1.7 Hz), 8.27 (1H, s), 8.82 (1H, s), 11.93 (1H, s)

### 8-(2-Methoxyphenyl)-2-((2-methoxyphenyl)amino)-7-oxo-7,8-dihydropyrido[2,3-d]pyrimidin-5-yl trifluoromethanesulfonate

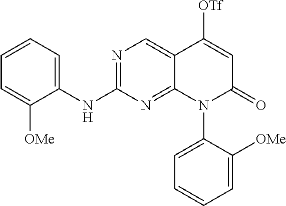 [SMILES: COc1ccccc1Nc1ncc2c(OS(=O)(=O)C(F)(F)F)cc(=O)n(-c3ccccc3OC)c2n1]

General Procedure 8 was applied to 5-hydroxy-8-(2-methoxyphenyl)-2-((2-methoxyphenyl)amino)pyrido[2,3-d]pyrimidin-7(8H)-one (135 mg, 0.346 mmol) with triethylamine (96.3 μL, 0.692 mmol), triflic anhydride (87.2 μL, 0.519 mmol) and dichloromethane (3.8 mL). The crude product was purified by flash column chromatography eluting with ethyl acetate (0-100%) in 40-60 petroleum ether to yield the title compound as a beige solid (105 mg, 0.201 mmol, 58%).

(ES, m/z): [M+H]⁺=523.3

¹H NMR (500 MHz, DMSO-d₆) δ_{H} 3.67 (3H, s), 3.79 (3H, s), 6.53 (1H, s), 6.71-6.74 (1H, m), 6.95-7.01 (2H, m), 7.13 (1H, td, J=7.6, 1.2 Hz), 7.27 (1H, dd, J=8.4, 1.2 Hz), 7.31-7.38 (2H, m), 7.55 (1H, td, J=8.1, 1.7 Hz), 8.78 (1H, s), 10.15 (1H, s)

### 8-(2-Methoxyphenyl)-2-((2-methoxyphenyl)amino)-5-vinylpyrido[2,3-d]pyrimidin-7(8H)-one

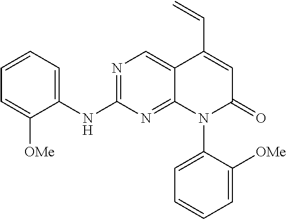 [SMILES: C=Cc1cc(=O)n(-c2ccccc2OC)c2nc(Nc3ccccc3OC)ncc12]

General Procedure 19 was applied to 8-(2-methoxyphenyl)-2-((2-methoxyphenyl)amino)-7-oxo-7,8-dihydropyrido[2,3-d]pyrimidin-5-yl trifluoromethanesulfonate (97.0 mg, 0.186 mmol) with bis(triphenylphosphine) palladium(II) dichloride (6.51 mg, 9.29 μmol), vinyl tributyltin (76.2 μL, 0.260 mmol) and toluene (1.30 mL). The crude product was purified by flash column chromatography eluting with ethyl acetate (0-100%) in 40-60 petroleum ether to yield the title compound as a beige solid (18.4 mg, 45.9 μmol, 25%).

(ES, m/z): [M+H]⁺=401.3

¹H NMR (500 MHz, DMSO-d₆) δ_{H} 3.65 (3H, s), 3.82 (3H, s), 5.75 (1H, dd, J=11.1, 1.1 Hz), 6.18 (1H, dd, J=17.2, 1.1 Hz), 6.49 (1H, t, J=7.6 Hz), 6.64 (1H, d, J=0.7 Hz), 6.86-6.95 (2H, m), 7.14 (1H, td, J=7.6, 1.2 Hz), 7.28 (2H, ddd, J=8.4, 3.4, 1.5 Hz), 7.30-7.38 (1H, m), 7.39-7.47 (1H, m), 7.55 (1H, ddd, J=8.3, 7.5, 1.7 Hz), 8.26 (1H, s), 9.03 (1H, s).

### Example 154

### Methyl 2-chloro-4-((3,4-dimethoxyphenyl)amino)pyrimidine-5-carboxylate

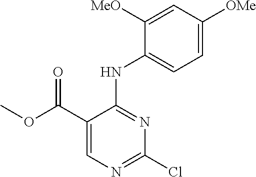 [SMILES: COC(=O)c1cnc(Cl)nc1Nc1ccc(OC)cc1OC]

A solution of methyl 2,4-dichloropyrimidine-5-carboxylate (15.0 g, 72.5 mmol, 1.0 eq.), N,N-diisopropylethylamine (18.7 g, 145 mmol, 2.0 eq.) and 2,4-dimethoxyaniline (12.2 g, 79.7 mmol, 1.1 eq.) in DMF (150 mL) was stirred overnight at room temperature. The resulting mixture was diluted with water (300 mL) and extracted with ethyl acetate (3×80 mL).

The combined organic layers were washed with brine (2×50 mL), dried (Na₂SO₄) and concentrated under reduced pressure. The residue was purified by flash column chromatography, eluting with ethyl acetate (50%) in 40-60 petroleum ether to afford the title compound as a light yellow solid (19.0 g, 82%).

### Methyl 4-((2,4-dimethoxyphenyl)amino)-2-((2-methoxyphenyl)amino)pyrimidine-5-carboxylate

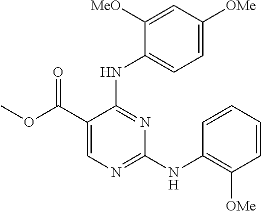 [SMILES: COC(=O)c1cnc(Nc2ccccc2OC)nc1Nc1ccc(OC)cc1OC]

A solution of methyl 2-chloro-4-((3,4-dimethoxyphenyl)amino)pyrimidine-5-carboxylate (19.0 g, 58.8 mmol, 1.0 eq.), potassium carbonate (16.2 g, 117 mmol, 2.0 eq.) and 2-methoxyaniline (7.96 g, 64.7 mmol, 1.1 eq.) in NMP (100 mL) was stirred overnight at 120° C. The resulting mixture was diluted with water (200 mL) and extracted with ethyl acetate (4×50 mL). The combined organic layers were washed with brine (2×50 mL), dried (Na₂SO₄) and concentrated under reduced pressure. The residue was purified by flash column chromatography, eluting with ethyl acetate (50%) in 40-60 petroleum ether to yield the title compound as a yellow solid (10.0 g, 40%).

### 4-((2,4-dimethoxyphenyl)amino)-2-((2-methoxyphenyl)amino)pyrimidine-5-carboxylic acid

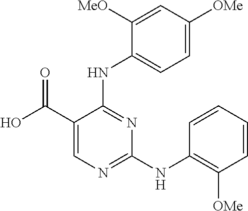 [SMILES: COc1ccc(Nc2nc(Nc3ccccc3OC)ncc2C(=O)O)c(OC)c1]

A solution of methyl 4-((2,4-dimethoxyphenyl)amino)-2-((2-methoxyphenyl)amino)pyrimidine-5-carboxylate (10.0 g, 24.4 mmol, 1.0 eq.) and lithium hydroxide (1.17 g, 48.8 mmol, 2 eq.) in THF and water (100 mL, 5:1). was stirred overnight at room temperature. The resulting mixture was concentrated under reduced pressure then diluted with water (100 mL) and acidified to pH 3 with 4M aqueous hydrochloric acid. The precipitated solids were collected by filtration, washing with water (3×30 mL). The resulting solid was dried under reduced pressure to yield the title compound as a white solid (7.00 g, 72%).

### (1H-benzo[d][1,2,3]triazol-1-yl)(4-((2,4-dimethoxyphenyl)amino)-2-((2-methoxyphenyl)amino)pyrimidin-5-yl)methanone

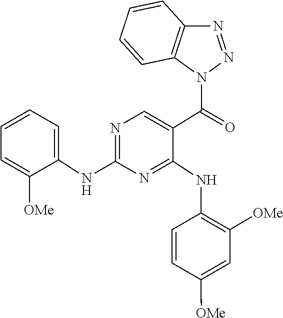 [SMILES: COc1ccc(Nc2nc(Nc3ccccc3OC)ncc2C(=O)n2nnc3ccccc32)c(OC)c1]

General procedure 4 was applied to 4-((2,4-dimethoxyphenyl)amino)-2-((2-methoxyphenyl)amino)pyrimidine-5-carboxylic acid (7.00 g, 17.7 mmol), EDCl (3.37 g, 17.7 mmol) and benzotriazole (2.10 g, 17.7 mmol) in dichloromethane (100 mL). The residue was purified by trituration with dichloromethane (30 mL). The precipitated solids were collected by filtration and washed with dichloromethane (3×10 mL). The resulting solid was dried under reduced pressure to yield the title compound as a yellow solid (3.00 g, 34%).

### tert-butyl 8-(2,4-dimethoxyphenyl)-2-[(2-methoxyphenyl)amino]-5,7-dioxo-6H-pyrido[2,3-d]pyrimidine-6-carboxylate

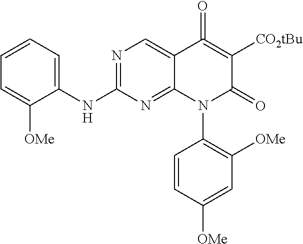

To a solution of 1-tert-butyl 3-ethylpropanedioate (227 mg, 1.21 mmol, 1.2 eq.) in THF (5.0 mL) was added sodium hydride (36.2 mg, 1.51 mmol, 1.5 eq.) at 0° C. The mixture was stirred for 30 minutes then 5-(1,2,3-benzotriazole-1-carbonyl)-N⁴-(2,4-dimethoxyphenyl)-N²-(2-methoxyphenyl)pyrimidine-2,4-diamine (500 mg, 1.01 mmol, 1.0 eq.) in DMF (5.0 mL) was added at 0° C. dropwise. The resulting mixture was warmed to room temperature and stirred for 4 h. The reaction mixture was quenched with water (50 mL) and then the pH was adjusted to 2 with 3N aqueous hydrochloric acid at 0° C. The solids were collected by filtration, washing with water and 40-60 petroleum ether. The filter cake was dried under reduced pressure to yield the title compound as a yellow solid (300 mg, 57%).

### 8-(2,4-dimethoxyphenyl)-2-[(2-methoxyphenyl)amino]-6H-pyrido[2,3-d]pyrimidine-5,7-dione

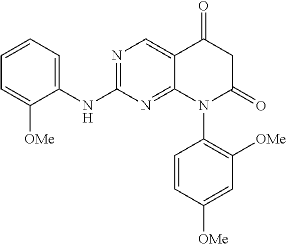 [SMILES: COc1ccc(N2C(=O)CC(=O)c3cnc(Nc4ccccc4OC)nc32)c(OC)c1]

To a stirred solution of tert-butyl 8-(2,4-dimethoxyphenyl)-2-[(2-methoxyphenyl)amino]-5,7-dioxo-6H-pyrido[2,3-d]pyrimidine-6-carboxylate (300 mg, 0.576 mmol, 1.0 eq.) in trifluoroacetic acid (3.0 mL). The resulting mixture was stirred overnight at 80° C. then concentrated under reduced pressure. The residue was purified by revers phase flash column chromatography eluting with acetonitrile (20-100%) in water (0.1% NH₄HCO₃) to yield the title compound as a light yellow oil (200 mg, 82%).

### 8-(2,4-dimethoxyphenyl)-2-[(2-methoxyphenyl)amino]-7-oxopyrido[2,3-d]pyrimidin-5-yl trifluoromethanesulfonate

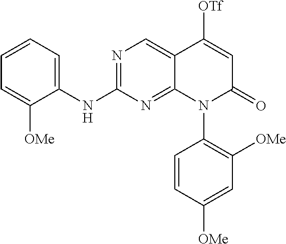 [SMILES: COc1ccc(-n2c(=O)cc(OS(=O)(=O)C(F)(F)F)c3cnc(Nc4ccccc4OC)nc32)c(OC)c1]

General procedure 8 was applied to 8-(2,4-dimethoxyphenyl)-2-[(2-methoxyphenyl)amino]-6H-pyrido[2,3-d]pyrimidine-5,7-dione (200 mg, 0.476 mmol), triethylamine (96.3 mg, 0.952 mmol) and triflic anhydride (161 mg, 0.571 mmol) in dichloromethane (2.0 mL). The residue was purified by reverse phase flash column chromatography eluting with acetonitrile (30-100%) in water (0.1% NH₄HCO₃) to yield the title compound as a light yellow solid (100 mg, 38%).

### 8-(2,4-dimethoxyphenyl)-5-ethenyl-2-[(2-methoxyphenyl)amino]pyrido[2,3-d]pyrimidin-7-one

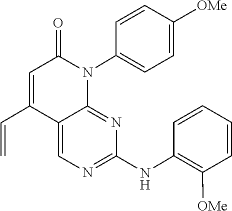 [SMILES: C=Cc1cc(=O)n(-c2ccc(OC)cc2)c2nc(Nc3ccccc3OC)ncc12]

To a solution of 8-(2,4-dimethoxyphenyl)-2-[(2-methoxyphenyl)amino]-7-oxopyrido[2,3-d]pyrimidin-5-yl trifluoromethanesulfonate (100 mg, 0.181 mmol, 1.0 eq.) and 2-ethenyl-4,4,5,5-tetramethyl-1,3,2-dioxaborolane (33.5 mg, 0.217 mmol, 1.2 eq.) in dioxane (1.0 mL) and water (0.20 mL) was added potassium carbonate (37.5 mg, 0.272 mmol, 1.5 eq.) and (1,1′-bis(diphenylphosphino)ferrocene)palladium(II) dichloride (6.62 mg, 9.00 μmol, 0.05 eq.). After stirring for 12 h at 100° C., the mixture was concentrated under reduced pressure. The crude product was purified by Prep-HPLC eluting with acetonitrile (40-65%) in water (0.1% NH₄CO₃) to yield the title compound as an off-white solid (5.00 mg, 6%).

(ES, m/z): [M+H]⁺=431

¹H NMR (300 MHz, Methanol-d₄) δ 8.95 (s, 1H), 7.63 (d, J=8.2 Hz, 1H), 7.35-7.19 (m, 1H), 7.18 (d, J=8.5 Hz, 1H), 6.99-6.86 (m, 2H), 6.86-6.73 (m, 2H), 6.64-6.62 (m, 1H), 6.56-6.53 (m, 1H), 6.14-6.08 (m, 1H), 5.81-5.77 (m, 1H), 3.97 (s, 3H), 3.91 (s, 3H), 3.70 (s, 3H).

### Example 155

### 8-(2,4-dimethoxyphenyl)-2-methanesulfonyl-5-[2-(triisopropylsilyl)ethynyl]pyrido[2,3-d]pyrimidin-7-one

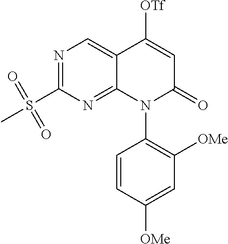 [SMILES: COc1ccc(-n2c(=O)cc(OS(=O)(=O)C(F)(F)F)c3cnc(S(C)(=O)=O)nc32)c(OC)c1]

General procedure 10 was applied to 8-(2,4-dimethoxyphenyl)-2-(methylsulfanyl)-7-oxopyrido[2,3-d]pyrimidin-5-yl trifluoromethanesulfonate (1.00 g, 2.09 mmol) and m-CPBA (1.27 g, 7.33 mmol) in dichloromethane (20 mL). The residue was purified by flash column chromatography, eluting with ethyl acetate (15%) in 40-60 petroleum ether to afford the title compound as a light yellow solid (600 mg, 53%).

(ES, m/z): [M+H]⁺=510.4

### 8-(2,4-dimethoxyphenyl)-2-((4-((2-(dimethylamino)ethyl)(methyl)amino)phenyl)amino)-7-oxo-7,8-dihydropyrido[2,3-d]pyrimidin-5-yl trifluoromethanesulfonate

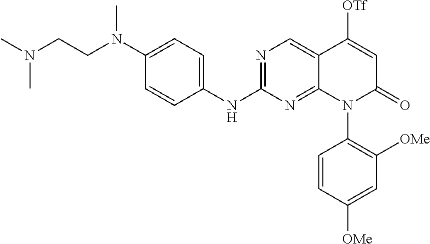 [SMILES: COc1ccc(-n2c(=O)cc(OS(=O)(=O)C(F)(F)F)c3cnc(Nc4ccc(N(C)CCN(C)C)cc4)nc32)c(OC)c1]

General procedure 13 was applied to 8-(2,4-dimethoxyphenyl)-2-methanesulfonyl-7-oxopyrido[2,3-d]pyrimidin-5-yl trifluoromethanesulfonate (600 mg, 1.18 mmol), trifluoroacetic acid (134 mg, 1.18 mmol) and N¹-[2-(dimethylamino)ethyl]-N¹-methylbenzene-1,4-diamine (227 mg, 1.18 mmol) in 2-butanol (4.0 mL). The resulting mixture was stirred for 1 h at 110° C. The residue was purified by reverse phase flash column chromatography eluting with acetonitrile (20-100%) in to yield the title compound as a light brown solid (400 mg, 55%).

(ES, m/z): [M+H]⁺=623.6

### 8-(2,4-dimethoxyphenyl)-2-[(4-{[2-(dimethylamino)ethyl](methyl)amino}phenyl)amino]-5-ethenylpyrido[2,3-d]pyrimidin-7-one

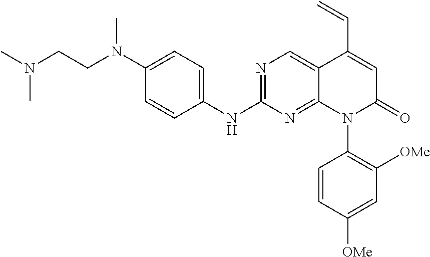 [SMILES: C=Cc1cc(=O)n(-c2ccc(OC)cc2OC)c2nc(Nc3ccc(N(C)CCN(C)C)cc3)ncc12]

To a solution of 8-(2,4-dimethoxyphenyl)-2-[(4-{[2-(dimethylamino)ethyl](methyl)amino}phenyl)amino]-7-oxopyrido[2,3-d]pyrimidin-5-yl trifluoromethanesulfonate (140 mg, 0.225 mmol, 1.0 eq.) and tributyl(ethenyl)stannane (71.3 mg, 0.225 mmol, 1.0 eq.) in DMF (3.0 mL) was added tetrakis(triphenylphosphine)palladium(0) (25.9 mg, 23.0 μmol, 0.1 eq.). After stirring for 1 h at 100° C., the resulting mixture was concentrated under reduced pressure. The residue was purified by Prep-TLC column chromatography, eluting with acetonitrile (50%) in water to afford the title compound as a yellow solid (20.2 mg, 18%).

(ES, m/z): [M+H]⁺=501.3

¹H NMR (300 MHz, DMSO-d6) δ 9.77 (s, 1H), 8.92 (s, 1H), 7.30 (dd, J=17.2, 11.1 Hz, 1H), 7.15 (dd, J=9.5, 6.3 Hz, 3H), 6.81 (d, J=2.6 Hz, 1H), 6.71 (dd, J=8.6, 2.6 Hz, 1H), 6.51 (s, 1H), 6.36 (d, J=8.5 Hz, 2H), 6.13 (dd, J=17.2, 1.2 Hz, 1H), 5.71 (dd, J=10.9, 1.2 Hz, 1H), 3.90 (s, 3H), 3.33 (s, 2H), 2.82 (s, 3H), 2.36 (t, J=7.1 Hz, 2H), 2.21 (s, 6H).

### Example 156

### Ethyl 2-chloro-4-((3-methoxyphenyl) amino)pyrimidine-5-carboxylate

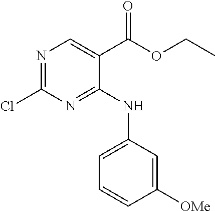 [SMILES: CCOC(=O)c1cnc(Cl)nc1Nc1cccc(OC)c1]

To a solution of m-anisidine (2.02 mL, 18.1 mmol, 1.0 eq.) and N,N-diisopropylethylamine (4.00 mL, 23.0 mmol, 1.3 eq.) in THF (100 mL) at 0° C. was added ethyl 2,4-dichloropyrimidine-5-carboxylate (4.00 g, 18.1 mmol, 1.0 eq.). The reaction mixture was stirred overnight at room temperature. The solvent was removed under reduced pressure. The crude material was purified by flash column chromatography eluting with ethyl acetate (0-100%) in 40-60 petroleum ether to yield the title compound as a grey solid (5.40 g, 17.5 mmol, 97%).

(ES, m/z): [M+H]⁺=308.2

¹H NMR (500 MHz, CDCl₃) δ 1.35 (2H, t, J=7.1 Hz), 3.76 (3H, s), 4.33 (2H, q, J=7.1 Hz), 6.71 (1H, ddd, J=8.2, 2.4, 0.9 Hz), 7.15 (1H, dq, J=8.0, 0.9 Hz), 7.17-7.23 (1H, m), 7.43 (1H, t, J=2.2 Hz), 8.71 (1H, s), 10.35 (1H, s)

### Ethyl 2-((2-methoxyphenyl)amino)-4-((3-methoxyphenyl)amino)pyrimidine-5-carboxylate

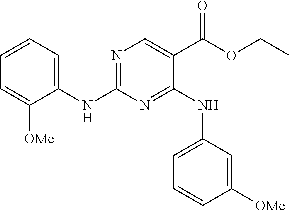 [SMILES: CCOC(=O)c1cnc(Nc2ccccc2OC)nc1Nc1cccc(OC)c1]

To a solution of ethyl 2-chloro-4-((3-methoxyphenyl)amino)pyrimidine-5-carboxylate (5.40 g, 17.5 mmol) in a mixture of ethanol and THF (251 mL: 97.0 mL) was added o-anisidine (3.80 mL, 33.7 mmol) and 1M aqueous hydrochloric acid (1.8 mL). The mixture was heated at 80° C. for 1 h, then cooled to ambient temperature. The product was collected by filtration and washed with ethanol to yield the title compound as an off-white powder (6.20 g, 15.7 mmol, 90%).

(ES, m/z): [M+H]⁺=395.3

¹H NMR (500 MHz, CDCl₃) δ_{H} 1.34 (3H, t, J=7.1 Hz), 3.69 (3H, s), 3.82 (3H, s), 4.30 (2H, q, J=7.1 Hz), 6.67 (1H, dd, J=8.5, 2.4 Hz), 6.81 (2H, ddd, J=12.0, 7.7, 4.5 Hz), 6.92-7.01 (1H, m), 7.11 (1H, d, J=7.9 Hz), 7.16 (1H, s), 7.18-7.19 (1H, m), 8.23 (1H, d, J=6.7 Hz), 8.69 (1H, s), 10.35 (1H, s), NH not visualised.

### 2-((2-Methoxyphenyl)amino)-4-((3-methoxyphenyl)amino)pyrimidine-5-carboxylic acid

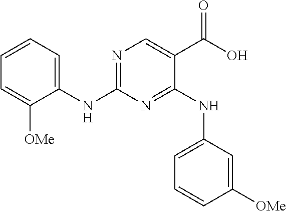 [SMILES: COc1cccc(Nc2nc(Nc3ccccc3OC)ncc2C(=O)O)c1]

1M aqueous sodium hydroxide (102 mL, 102 mmol, 6.5 eq.) was slowly added into a solution of ethyl 2-((2-methoxyphenyl)amino)-4-((3-methoxyphenyl)amino)pyrimidine-5-carboxylate (6.20 g, 15.7 mmol, 1.0 eq.) in THF (102 mL), then the mixture was stirred at 80° C. overnight. The solution was acidified to PH 4-5 with 4M aqueous hydrochloric acid after the solvent was partly removed under reduced pressure. The resulting solid was collected by vacuum filtration washing with water and dried in a vacuum oven (40° C.) to give the title compound as a white solid (5.70 g, 15.6 mmol, 99%).

(ES, m/z): [M+H]⁺=367.3

¹H NMR (500 MHz, DMSO-d₆) δ_{H} 3.67 (3H, s), 3.79 (3H, s), 6.66 (1H, dq, J=6.8, 2.3 Hz), 6.87-6.94 (1H, dq, J=6.8, 2.3 Hz), 7.07 (1H, dd, J=8.2, 1.4 Hz), 7.12-7.32 (3H, m), 7.62-7.79 (1H, m), 8.69 (2H, m), 8.82 (1H, s), 10.25 (1H, s), COOH not visualised.

### 2-((2-Methoxyphenyl)amino)-4-((3-methoxyphenyl)amino)pyrimidine-5-carbonyl chloride

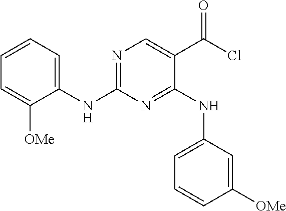 [SMILES: COc1cccc(Nc2nc(Nc3ccccc3OC)ncc2C(=O)Cl)c1]

To a solution of 2-((2-methoxyphenyl)amino)-4-((3-methoxyphenyl)amino)pyrimidine-5-carboxylic acid (5.70 g, 15.6 mmol) in dichloromethane (156 mL) was carefully added oxalyl chloride (1.6 mL) and DMF (1.6 mL). The resulting mixture was stirred at room temperature overnight. The solvent was evaporated to yield the title compound as a white solid (5.60 g, 14.6 mmol, 94%), which was used without further purification.

### 5-Hydroxy-8-(3-methoxyphenyl)-2-((2-methoxyphenyl)amino)pyrido[2,3-d]pyrimidin-7(8H)-one

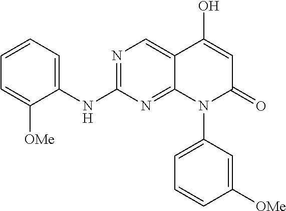 [SMILES: COc1cccc(-n2c(=O)cc(O)c3cnc(Nc4ccccc4OC)nc32)c1]

General Procedure 5 was applied to 2-((2-methoxyphenyl)amino)-4-((3-methoxyphenyl)amino)pyrimidine-5-carbonyl chloride (5.60 g, 14.6 mmol) in THF (15 mL), with 1M LiHMDS in THF (65.7 mL, 65.7 mmol), ethyl acetate (2.85 mL, 29.2 mmol) and THF (44 mL) to afford a brown solid (2.38 g). General procedure 6 was applied to the brown solid (2.38 g, 5.50 mmol) with N,N-diisopropylethylamine (6.7 mL) and DBU (0.82 mL) to give the title compound as a beige solid (2.00 mg, 5.12 mmol, 35%).

(ES, m/z): [M+H]⁺=391.2

¹H NMR (500 MHz, DMSO-d₆) δ_{H} 3.76 (3H, s), 3.81 (3H, s), 5.71 (1H, s), 6.80-6.86 (1H, m), 6.87-6.98 (2H, m), 6.95 (1H, dd, J=8.2, 1.6 Hz), 7.06-7.13 (1H, m), 7.41-7.48 (2H, m), 7.51 (1H, dd, J=8.1, 1.5 Hz), 8.25 (1H, s), 8.81 (1H, s4), 11.97 (1H, s)

### 8-(3-Methoxyphenyl)-2-((2-methoxyphenyl)amino)-5-vinylpyrido[2,3-d]pyrimidin-7(8H)-one

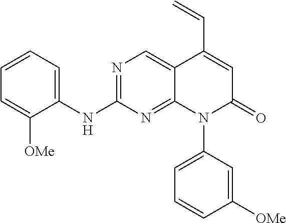 [SMILES: C=Cc1cc(=O)n(-c2cccc(OC)c2)c2nc(Nc3ccccc3OC)ncc12]

General Procedure 8 was applied to 5-hydroxy-8-(3-methoxyphenyl)-2-((2-methoxyphenyl)amino)pyrido[2,3-d]pyrimidin-7(8H)-one (2.00 g, 5.12 mmol) with triethylamine (1.42 mL, 10.2 mmol), triflic anhydride (2.98 mL, 7.68 mmol) and dichloromethane (56 mL). The crude product was purified by flash column chromatography eluting with ethyl acetate (0-100%) in 40-60 petroleum ether to yield 8-(3-methoxyphenyl)-2-((2-methoxyphenyl)amino)-7-oxo-7,8-dihydropyrido[2,3-d]pyrimidin-5-yl trifluoromethanesulfonate as a brown solid (1.68 g, 1.52 mmol, 30%).

General procedure 19 was applied to 8-(3-methoxyphenyl)-2-((2-methoxyphenyl)amino)-7-oxo-7,8-dihydropyrido[2,3-d]pyrimidin-5-yl trifluoromethanesulfonate (800 mg, 1.52 mmol) with bis(triphenylphosphine)palladium (II) dichloride (53.3 mg, 0.0760 mmol), vinyltributyltin (0.625 mL, 2.13 mmol) and toluene (11 mL).

The crude product was purified by flash column chromatography eluting with ethyl acetate (0-100%) in 40-60 petroleum ether to yield the title compound as an off-white solid (91.0 mg, 0.227 mmol, 15%).

(ES, m/z): [M+H]⁺=401.2

¹H NMR (500 MHz, DMSO-d₆) b 3.77 (3H, s), 3.82 (3H, s), 5.74 (1H, dd, J=11.0, 1.0 Hz), 6.18 (1H, dd, J=17.2, 1.1 Hz), 6.39-6.48 (1H, m), 6.65 (1H, d, J=0.7 Hz), 6.90 (2H, ddd, J=7.8, 1.9, 0.9 Hz), 6.96 (1H, dd, J=8.3, 1.6 Hz), 6.98 (1H, t, J=2.2 Hz), 7.13 (1H, ddd, J=8.4, 2.5, 0.9 Hz), 7.34 (1H, dd, J=17.3, 11.0 Hz), 7.46-7.53 (2H, m), 8.28 (1H, s), 9.04 (1H, s)

### Example 157

### Ethyl 2-chloro-4-((4-methoxyphenyl)amino)pyrimidine-5-carboxylate

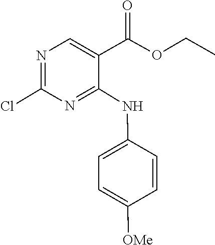 [SMILES: CCOC(=O)c1cnc(Cl)nc1Nc1ccc(OC)cc1]

To a solution of p-anisidine (1.67 g, 13.6 mmol, 1.0 eq.) and N,N-diisopropylethylamine (3.00 mL, 17.3 mmol, 1.3 eq.) in THF (75 mL) at 0° C., ethyl 2,4-dichloropyrimidine-5-carboxylate (3.00 g, 13.6 mmol, 1.0 eq.) was added. The reaction mixture was stirred overnight at room temperature. Excess THF was evaporated under reduced pressure. The crude material was purified by flash column chromatography eluting with ethyl acetate (0-100%) in 40-60 petroleum ether to yield the title compound as a light brown solid (2.40 g, 7.80 mmol, 57%).

(ES, m/z): [M+H]⁺=308.2

¹H NMR (500 MHz, CDC₃) δ 1.36 (3H, t, J=7.1 Hz), 3.75 (1H, s), 4.35 (2H, q, J=7.1 Hz), 6.79-6.91 (2H, m), 7.39-7.50 (2H, m), 8.72 (1H, s), 10.22 (1H, s)

### Ethyl 2-((2-methoxyphenyl)amino)-4-((4-methoxyphenyl)amino)pyrimidine-5-carboxylate

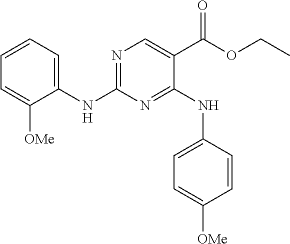 [SMILES: CCOC(=O)c1cnc(Nc2ccccc2OC)nc1Nc1ccc(OC)cc1]

To a solution of ethyl 2-chloro-4-((4-methoxyphenyl)amino)pyrimidine-5-carboxylate (2.40 g, 7.80 mmol, 1.0 eq.) in a mixture of ethanol and THF (111 mL: 43.0 mL) was added o-anisidine (1.68 mL, 14.9 mmol, 2.0 eq.) and 1M aqueous hydrochloric acid (0.78 mL). The mixture was heated at 80° C. for 3 h, then cooled to ambient temperature. The resulting solid was collected by vacuum filtration washing with ethanol (200 mL) to yield the title compound as an off-white powder (2.50 g, 6.34 mmol, 81%).

(ES, m/z): [M+H]⁺=395.2

¹H NMR (500 MHz, CDCl₃) δ 1.34 (3H, t, J=7.1 Hz), 3.78 (3H, s), 3.81 (3H, s), 4.29 (2H, q, J=7.1 Hz), 6.75 (1H, t, J=8.0 Hz), 6.80 (1H, dd, J=8.2, 1.4 Hz), 6.83-6.88 (2H, m), 6.91 (1H, ddd, J=8.1, 7.4, 1.6 Hz), 7.31-7.56 (2H, m), 7.91 (1H, s), 8.21 (1H, d, J=8.1 Hz), 8.68 (1H, s), 10.08 (1H, s)

### 2-((2-Methoxyphenyl)amino)-4-((4-methoxyphenyl)amino)pyrimidine-5-carboxylic acid

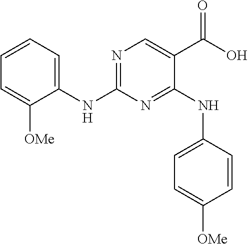 [SMILES: COc1ccc(Nc2nc(Nc3ccccc3OC)ncc2C(=O)O)cc1]

1M aqueous sodium hydroxide solution (41.2 mL, 41.2 mmol, 6.5 eq.) was slowly added into a solution of ethyl 2-((2-methoxyphenyl)amino)-4-((4-methoxyphenyl)amino)pyrimidine-5-carboxylate (2.50 g, 6.34 mmol, 1.0 eq.) in THF (41 mL), then the mixture was stirred at 80° C. overnight. The solution was acidified to PH 4-5 with 4M aqueous hydrochloric acid after the solvent was partly removed under reduced pressure. The resulting solid was collected by vacuum filtration washing with water and dried in a vacuum oven (40° C.) to give the title compound as a white solid (2.20 g, 6.00 mmol, 95%).

(ES, m/z): [M+H]⁺=367.2

¹H NMR (500 MHz, DMSO-d₆) δ_{H} 3.76 (3H, s), 3.81 (3H, s), 6.87 (3H, dd, J=7.8, 5.1 Hz), 7.01-7.21 (2H, m), 7.49-7.56 (2H, m), 7.77 (1H, d, J=7.9 Hz), 8.64 (1H, s), 10.35 (1H, s), 13.06 (1H, s), COOH not visualised.

### 2-((2-Methoxyphenyl)amino)-4-((4-methoxyphenyl)amino)pyrimidine-5-carbonyl chloride

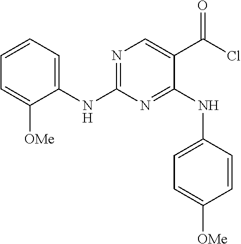 [SMILES: COc1ccc(Nc2nc(Nc3ccccc3OC)ncc2C(=O)Cl)cc1]

To a solution of 2-((2-methoxyphenyl)amino)-4-((4-methoxyphenyl)amino)pyrimidine-5-carboxylic acid (2.20 g, 6.00 mmol) in toluene (3.0 mL) was added thionyl chloride (10 mL). The resulting mixture was stirred at room temperature for 1 h. The solvent was evaporated to yield the title compound as a white solid (2.20 g, 5.72 mmol, 95%), which was used without further purification.

### 5-Hydroxy-8-(4-methoxyphenyl)-2-((2-methoxyphenyl)amino)pyrido[2,3-d]pyrimidin-7(8H)-one

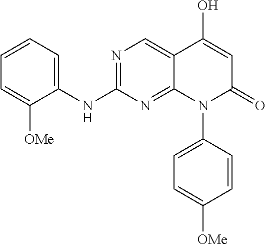 [SMILES: COc1ccc(-n2c(=O)cc(O)c3cnc(Nc4ccccc4OC)nc32)cc1]

General Procedure 5 was applied to a solution of 2-((2-methoxyphenyl)amino)-4-((4-methoxyphenyl)amino)pyrimidine-5-carbonyl chloride (2.20 g, 5.72 mmol) in anhydrous THF (5.7 mL) with 1M LiHMDS in THF (25.7 mL, 25.7 mmol), ethyl acetate (1.12 mL, 11.4 mmol) and THF (17 mL) to afford a brown solid (2.71 g). General procedure 6 was applied to the resulting solid (2.71 g, 6.20 mmol) with DBU (0.93 mL) and N,N-diisopropylethylamine (7.6 mL) to yield the title compound as a beige solid (1.93 mg, 4.94 mmol, 86%).

(ES, m/z): [M+H]⁺=391.2

¹H NMR (500 MHz, CDCl₃) b 3.78 (3H, s), 3.84 (3H, s), 6.65-6.76 (1H, m), 6.78-6.88 (4H, m), 7.09 (1H, ddd, J=8.8, 7.7, 1.5 Hz), 7.74 (1H, dt, J=8.0, 2.9 Hz), 7.29-7.38 (2H, m), 8.69 (1H, s), 10.16 (1H, s), 11.31 (1H, s)

### 8-(4-Methoxyphenyl)-2-((2-methoxyphenyl)amino)-7-oxo-7,8-dihydropyrido[2,3-d]pyrimidin-5-yl trifluoromethanesulfonate

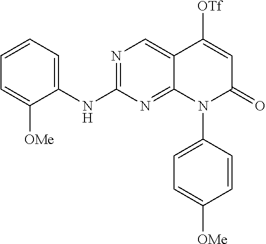 [SMILES: COc1ccc(-n2c(=O)cc(OS(=O)(=O)C(F)(F)F)c3cnc(Nc4ccccc4OC)nc32)cc1]

General Procedure 8 was applied to 5-hydroxy-8-(4-methoxyphenyl)-2-((2-methoxyphenyl)amino)pyrido[2,3-d]pyrimidin-7(8H)-one (1.93 g, 4.94 mmol) with triethylamine (1.37 mL, 9.88 mmol), triflic anhydride (1.24 mL, 7.41 mmol) and dichloromethane (54 mL). The crude product was purified by flash column chromatography eluting with ethyl acetate (0-50%) in 40-60 petroleum ether to yield the title compound as a dark brown solid (1.18 g, 2.26 mmol, 46%).

(ES, m/z): [M+H]⁺=523.2

¹H NMR (500 MHz, CDCl₃) δ_{H} 3.80 (3H, s), 3.86 (3H, s), 6.50 (1H, s), 6.76 (2H, d, J=8.1 Hz), 6.82-6.95 (2H, m), 7.03-7.09 (2H, m), 7.11-7.18 (2H, m), 8.14 (1H, s), 8.65 (1H, s)

### 8-(4-Methoxyphenyl)-2-((2-methoxyphenyl)amino)-5-vinylpyrido[2,3-d]pyrimidin-7(8H)-one

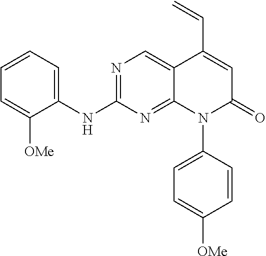 [SMILES: C=Cc1cc(=O)n(-c2ccc(OC)cc2)c2nc(Nc3ccccc3OC)ncc12]

General Procedure 19 was applied to 8-(4-methoxyphenyl)-2-((2-methoxyphenyl)amino)-7-oxo-7,8-dihydropyrido[2,3-d]pyrimidin-5-yl trifluoromethanesulfonate (306 mg, 0.586 mmol) with bis(triphenylphosphine) palladium(II) dichloride (20.5 mg, 29.3 μmol), vinyl tributyltin (0.240 mL, 0.820 mmol) and toluene (4.1 mL).

The crude product was purified by flash column chromatography eluting with ethyl acetate (0-100%) in 40-60 petroleum ether to yield the title compound as an off-white solid (75.0 mg, 0.187 mmol, 32%).

(ES, m/z): [M+H]⁺=401.3

¹H NMR (500 MHz, CDCl₃) δ_{H} 3.80 (3H, s), 3.86 (3H, s), 5.66 (1H, dd, J=11.0, 0.9 Hz), 5.92 (1H, dd, J=17.2, 0.9 Hz), 6.58 (1H, d, J=0.8 Hz), 6.74 (2H, dd, J=8.2, 1.4 Hz), 6.84 (2H, ddd, J=8.2, 7.4, 1.6 Hz), 6.94 (1H, ddd, J=17.2, 11.0, 0.9 Hz), 7.02-7.10 (2H, m), 7.12-7.17 (2H, m), 7.52 (1H, s), 8.68 (1H, s)

### Example 158

### N-[2-(methylsulfanyl)-7-oxo-8-phenyl-5-[2-(triisopropylsilyl)ethynyl]pyrido[2,3-d]pyrimidin-6-yl]acetamide

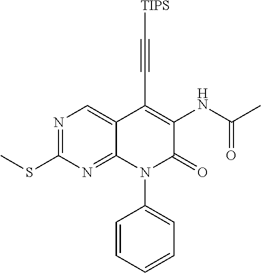

A solution of 6-amino-2-(methylsulfanyl)-8-phenyl-5-[2-(triisopropylsilyl)ethynyl]pyrido[2,3-d]pyrimidin-7-one (1.00 g, 2.15 mmol, 1.0 eq.), lithium chloride (90.0 mg, 2.15 mmol, 1.0 eq.) and acetic anhydride (330 mg, 3.23 mmol, 1.5 eq.) in THF (10 mL) was stirred for 2 h at 60° C. The mixture was cooled to room temperature. The residue was purified by reverse phase flash column chromatography eluting with acetonitrile (20-100%) in water to yield the title compound as a light yellow solid (850 mg, 78%).

(ES, m/z): [M+H]⁺=507.2

### N-{2-methanesulfonyl-7-oxo-8-phenyl-5-[2-(triisopropylsilyl)ethynyl]pyrido[2,3-d]pyrimidin-6-yl}acetamide

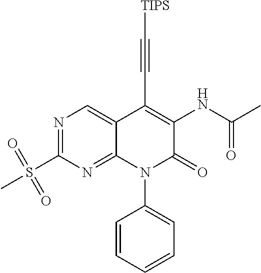

General procedure 10 was applied to N-[2-(methylsulfanyl)-7-oxo-8-phenyl-5-[2-(triisopropylsilyl)ethynyl]pyrido[2,3-d]pyrimidin-6-yl]acetamide (850 mg, 1.68 mmol) and m-CPBA (1.01 g, 5.87 mmol) in dichloromethane (20 mL). The residue was purified by reverse phase flash column chromatography eluting with acetonitrile (20-100%) in water to yield the title compound as a light yellow solid (800 mg, 88%).

(ES, m/z): [M+H]⁺=539.2

### N-{2-[(4-{[2-(dimethylamino)ethyl](methyl)amino}-2-methoxyphenyl)amino]-7-oxo-8-phenyl-5-[2-(triisopropylsilyl)ethynyl]pyrido[2,3-d]pyrimidin-6-yl}acetamide

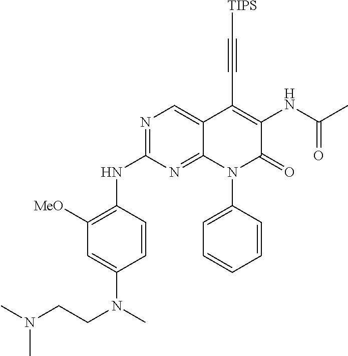

General procedure 13 was applied to N-{2-methanesulfonyl-7-oxo-8-phenyl-5-[2-(triisopropylsilyl)ethynyl]pyrido[2,3-d]pyrimidin-6-yl}acetamide (500 mg, 0.928 mmol), N¹-[2-(dimethylamino)ethyl]-3-methoxy-N¹-methylbenzene-1,4-diamine (207 mg, 0.928 mmol) and trifluoroacetic acid (106 mg, 0.928 mmol) in 2-butanol (5.0 mL). The resulting mixture was stirred for 1 h at 100° C. The residue was purified by reverse phase flash column chromatography eluting with acetonitrile (10-100%) in water to yield the title compound as a reddish solid (450 mg, 71%).

(ES, m/z): [M+H]⁺=682.4

### N-{2-[(4-{[2-(dimethylamino)ethyl](methyl)amino}-2-methoxyphenyl)amino]-5-ethynyl-7-oxo-8-phenylpyrido[2,3-d]pyrimidin-6-yl}acetamide

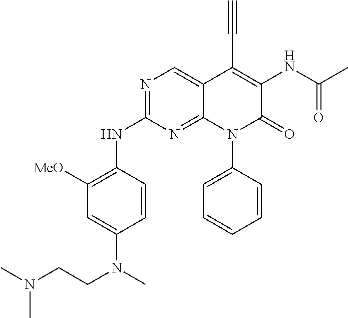 [SMILES: C#Cc1c(NC(C)=O)c(=O)n(-c2ccccc2)c2nc(Nc3ccc(N(C)CCN(C)C)cc3OC)ncc12]

General procedure 13 was applied to N-{2-[(4-{[2-(dimethylamino)ethyl](methyl)amino}-2-methoxyphenyl)amino]-7-oxo-8-phenyl-5-[2-(triisopropylsilyl)ethynyl]pyrido[2,3-d]pyrimidin-6-yl}acetamide (200 mg, 0.293 mmol) and potassium fluoride (341 mg, 5.86 mmol), THF (2.0 mL), water (1.0 mL) and methanol (0.20 mL). The resulting mixture was stirred for 2 h at 75° C. The residue was purified by reverse phase flash column chromatography eluting with acetonitrile (15-80%) in water to yield the title compound as a yellow green solid (110 mg, 71%).

(ES, m/z): [M+H]⁺=526.2

### N-{5-ethenyl-2-[(2-methoxyphenyl)amino]-7-oxo-8-phenylpyrido[2,3-d]pyrimidin-6-yl}acetamide

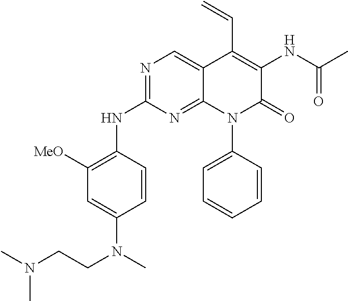 [SMILES: C=Cc1c(NC(C)=O)c(=O)n(-c2ccccc2)c2nc(Nc3ccc(N(C)CCN(C)C)cc3OC)ncc12]

To a solution of N-{5-ethynyl-2-[(2-methoxyphenyl)amino]-7-oxo-8-phenylpyrido[2,3-d]pyrimidin-6-yl}acetamide (50.0 mg, 0.118 mmol, 1.0 eq.) in methanol (40 mL) was added palladium on barium sulphate (5.70 mg, 24.0 μmol, 0.2 eq.) under nitrogen atmosphere. The mixture was hydrogenated at room temperature for 5 h, filtered through a Celite pad and concentrated under reduced pressure. The filtrate was purified by reverse phase flash column chromatography to yield the title compound as a light yellow solid (9.10 mg, 18%).

(ES, m/z): [M+H]⁺=428.2

¹H NMR (300 MHz, DMSO-d6) δ 9.40 (s, 1H), 8.85 (s, 1H), 8.25 (s, 1H), 7.66-7.54 (m, 3H), 7.44-7.32 (m, 3H), 7.02-6.77 (m, 3H), 6.46 (t, J=7.6 Hz, 1H), 5.77 (dd, J=23.7, 14.9 Hz, 2H), 3.82 (s, 3H), 2.00 (s, 3H).

### Example 159

### Methyl 2-chloro-4-(phenylamino)pyrimidine-5-carboxylate

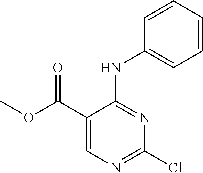 [SMILES: COC(=O)c1cnc(Cl)nc1Nc1ccccc1]

A solution of methyl 2,4-dichloropyrimidine-5-carboxylate (20.0 g, 96.6 mmol, 1.0 eq.), N,N-diisopropylethylamine (12.5 g, 96.8 mmol, 1.0 eq.), aniline (9.00 g, 96.7 mmol, 1.0 eq) in acetonitrile (200 mL) was stirred for 4 h at 70° C. The reaction mixture was cooled to room temperature and diluted with water (500 mL). The resulting solution was extracted with ethyl acetate (2×200 mL) and the combined organic layers dried (Na₂SO₄) and concentrated under reduced pressure. The crude material was purified by flash column chromatography eluting with ethyl acetate (0-25%) in 40-60 petroleum ether to yield the title compound as a light yellow solid (22.0 g, 86%).

### Ethyl 2-[(2-methoxyphenyl)amino]-4-(phenylamino)pyrimidine-5-carboxylate

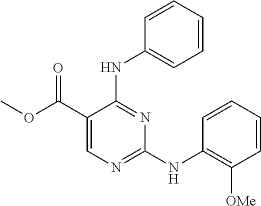 [SMILES: COC(=O)c1cnc(Nc2ccccc2OC)nc1Nc1ccccc1]

A solution of ethyl 2-chloro-4-(phenylamino)pyrimidine-5-carboxylate (22.0 g, 83.7 mmol, 1.0 eq.) N,N-diisopropylethylamine (10.8 g, 83.7 mmol, 1.0 eq.) and o-anisidine (10.3 g, 83.7 mmol, 1.0 eq.) in NMP (200 mL). The resulting solution was stirred for 12 h at 100° C. The reaction mixture was cooled, diluted with water (500 mL) and extracted with ethyl acetate (3×200 mL). The combined organic layers were dried (Na₂SO₄) and concentrated. The crude material was purified by flash column chromatography eluting with ethyl acetate (0-20%) in 40-60 petroleum ether to yield the title compound as a light yellow solid (15.0 g, 49%).

### 2-((2-methoxyphenyl)amino)-4-(phenylamino)pyrimidine-5-carboxylic acid

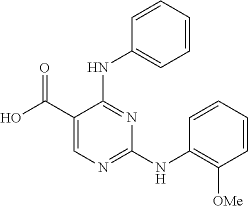 [SMILES: COc1ccccc1Nc1ncc(C(=O)O)c(Nc2ccccc2)n1]

A solution of ethyl 2-[(2-methoxyphenyl)amino]-4-(phenylamino)pyrimidine-5-carboxylate (15.0 g, 41.2 mmol, 1.0 eq.) and lithium hydroxide (3.09 g, 129 mmol, 3.0 eq.) in THF (15 mL) and water (5.0 mL) was stirred for 12 h at 50° C. The resulting mixture was concentrated under reduced pressure to remove most of THF. The pH value of the solution was adjusted to pH 2 with 2N aqueous hydrochloric acid aqueous solution. The solids were collected by filtration washing with water and methanol and then dried under vacuum to yield the title compound as a white solid (10.0 g, 72%).

### (1H-benzo[d][1,2,3]triazol-1-yl)(2-((2-methoxyphenyl)amino)-4-(phenylamino)pyrimidin-5-yl)methanone

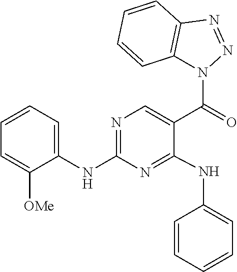 [SMILES: COc1ccccc1Nc1ncc(C(=O)n2nnc3ccccc32)c(Nc2ccccc2)n1]

General procedure 4 was applied to 2-((2-methoxyphenyl)amino)-4-(phenylamino)pyrimidine-5-carboxylic acid (10.0 g, 29.8 mmol), EDCl (5.68 g, 29.7 mmol) and 1H-benzo[d][1,2,3]triazole (3.54 g, 29.8 mmol) in dichloromethane (100 mL). The crude material was purified by reverse phase flash column chromatography eluting with acetonitrile (15-100%) in water (0.1% NH₄HCO₃) to yield the title compound an off-white solid (3.00 g, 23%).

### Ethyl 2-cyano-3-(2-((2-methoxyphenyl)amino)-4-(phenylamino)pyrimidin-5-yl)-3-oxopropanoate

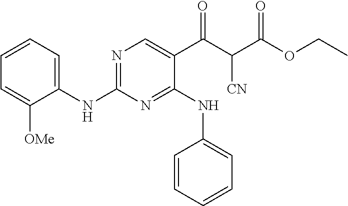 [SMILES: CCOC(=O)C(C#N)C(=O)c1cnc(Nc2ccccc2OC)nc1Nc1ccccc1]

To a solution of ethyl 2-cyanoacetate (1.16 g, 10.3 mmol, 1.5 eq.) in THF (15 mL) was added sodium hydride (60% in oil, 549 mg, 2.0 eq.) at 0° C. The mixture was stirred for 15 min. Then (1H-benzo[d][1,2,3]triazol-1-yl)(2-((2-methoxyphenyl)amino)-4-(phenylamino)pyrimidin-5-yl)methanone (3.00 g, 6.86 mmol, 1.0 eq.) in THF (30 mL) was added dropwise at 0° C. The resulting mixture was warmed to room temperature and stirred for 2 h. The reaction mixture was quenched by water (100 mL) and then acidified to pH 2 with 6N aqueous hydrochloric acid at 0° C. The solids were collected by filtration washing with water and 40-60 petroleum ether then dried under vacuum to yield the title compound as a light yellow solid (2.20 g, 74%) 5-hydroxy-2-((2-methoxyphenyl)amino)-7-oxo-8-phenyl-7,8-dihydropyrido[2,3-d]pyrimidine-6-carbonitrile

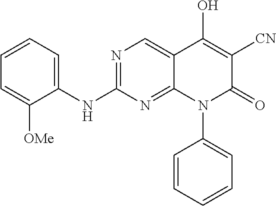 [SMILES: COc1ccccc1Nc1ncc2c(O)c(C#N)c(=O)n(-c3ccccc3)c2n1]

General procedure 6 was applied to ethyl 2-cyano-3-(2-((2-methoxyphenyl)amino)-4-(phenylamino)pyrimidin-5-yl)-3-oxopropanoate (2.20 g, 5.10 mmol), N,N-diisopropylethylamine (8.0 mL) and DBU (1.0 mL) to yield the title compound as light white solid (1.50 g, 76%).

### 6-Cyano-2-((2-methoxyphenyl)amino)-7-oxo-8-phenyl-7,8-dihydropyrido[2,3-d]pyrimidin-5-yl trifluoromethanesulfonate

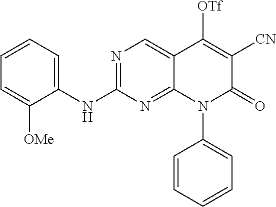 [SMILES: COc1ccccc1Nc1ncc2c(OS(=O)(=O)C(F)(F)F)c(C#N)c(=O)n(-c3ccccc3)c2n1]

General procedure 8 was applied to 5-hydroxy-2-((2-methoxyphenyl)amino)-7-oxo-8-phenyl-7,8-dihydropyrido[2,3-d]pyrimidine-6-carbonitrile (1.50 g, 3.89 mmol), triethylamine (983 mg, 9.72 mmol) and trifluoromethanesulfonic anhydride (1.64 g, 5.84 mmol) in dichloromethane (100 mL). The crude material was purified by flash column chromatography eluting with ethyl acetate (50%) in 40-60 petroleum ether (1:1) to yield the title compound as a light yellow solid (850 mg, 42%).

### 5-ethenyl-2-[(2-methoxyphenyl)amino]-7-oxo-8-phenylpyrido[2,3-d]pyrimidine-6-carbonitrile

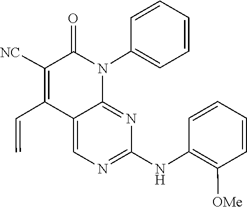 [SMILES: C=Cc1c(C#N)c(=O)n(-c2ccccc2)c2nc(Nc3ccccc3OC)ncc12]

A solution of 6-cyano-2-[(2-methoxyphenyl)amino]-7-oxo-8-phenylpyrido[2,3-d]pyrimidin-5-yl trifluoromethanesulfonate (200 mg, 0.387 mmol, 1.0 eq.), tributyl(ethenyl)stannane (245 mg, 0.773 mmol, 2.0 eq.) and tetrakis(triphenylphosphine)palladium(0) (44.6 mg, 39.0 μmol, 0.10 eq.) in dioxane (2.0 mL) was stirred for 2 h at 100° C. then concentrated under reduced pressure. The crude material was purified by flash column chromatography eluting with ethyl acetate (0-50%) in 40-60 petroleum ether to yield the title compound as a yellow solid (30.0 mg, 19%).

(ES, m/z): [M+H]⁺=396

¹H-NMR (300 MHz, DMSO-d₆) δ 8.99 (s, 1H), 8.85 (s, 1H), 7.62-7.52 (m, 3H), 7.40-7.35 (m, 2H), 7.32-7.21 (m, 2H), 6.97-6.94 (m, 2H), 6.47 (s, 1H), 6.26-6.11 (m, 2H), 3.80 (s, 3H).

### Example 160

### Methyl 2-chloro-4-((3,4-dimethoxyphenyl)amino)pyrimidine-5-carboxylate

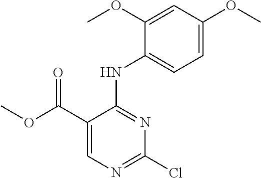

A solution of methyl 2,4-dichloropyrimidine-5-carboxylate (15.0 g, 72.4 mmol, 1.0 eq.), N,N-diisopropylethylamine (18.7 g, 145 mmol, 2.0 eq.) and 2,4-dimethoxyaniline (12.2 g, 79.7 mmol, 1.1 eq.) in DMF (150 mL) was stirred overnight at room temperature. The resulting mixture was diluted with water (300 mL) and extracted with ethyl acetate (3×80 mL). The combined organic layers were washed with brine (2×50 mL), dried (Na₂SO₄) and concentrated under reduced pressure. The residue was purified by flash column chromatography, eluting with ethyl acetate (50%) in 40-60 petroleum ether to afford the title compound as a light yellow solid (19.0 g, 82%).

### Methyl 4-((2,4-dimethoxyphenyl)amino)-2-((2-methoxyphenyl)amino)pyrimidine-5-carboxylate

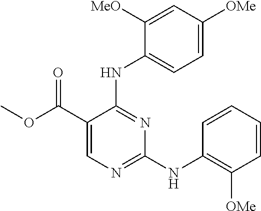 [SMILES: COC(=O)c1cnc(Nc2ccccc2OC)nc1Nc1ccc(OC)cc1OC]

A solution of methyl 2-chloro-4-((3,4-dimethoxyphenyl)amino)pyrimidine-5-carboxylate (19.0 g, 58.8 mmol, 1.0 eq.), potassium carbonate (16.2 g, 117 mmol, 2.0 eq.) and 2-methoxyaniline (7.96 g, 64.7 mmol, 1.1 eq.) in NMP (100 mL) was stirred overnight at 120° C. The resulting mixture was diluted with water (200 mL) and extracted with ethyl acetate (4×50 mL). The combined organic layers were washed with brine (2×50 mL), dried (Na₂SO₄) and concentrated under reduced pressure. The residue was purified by flash column chromatography, eluting with ethyl acetate (50%) in 40-60 petroleum ether to afford the title compound as a yellow solid (10.0 g, 40%).

### 4-((2,4-dimethoxyphenyl)amino)-2-((2-methoxyphenyl)amino)pyrimidine-5-carboxylic acid

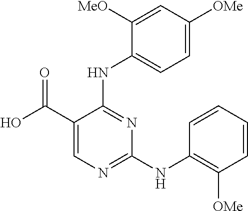 [SMILES: COc1ccc(Nc2nc(Nc3ccccc3OC)ncc2C(=O)O)c(OC)c1]

A solution of methyl 4-((2,4-dimethoxyphenyl)amino)-2-((2-methoxyphenyl)amino)pyrimidine-5-carboxylate (10.0 g, 24.4 mmol, 1.0 eq.), and lithium hydroxide (1.17 g, 48.8 mmol, 2.0 eq.) in THF and water (100 mL, 5:1) was stirred overnight at room temperature. The resulting mixture was concentrated under reduced pressure. The residue was diluted with water (100 mL), acidified to pH 3 with 4M aqueous hydrochloric acid and the resulting precipitated solids were collected by filtration, washed with water (3×30 mL), dried under vacuum to yield the title compound as a white solid (7.00 g, 72%).

### (1H-benzo[d][1,2,3]triazol-1-yl)(4-((2,4-dimethoxyphenyl)amino)-2-((2-methoxyphenyl)amino)pyrimidin-5-yl)methanone

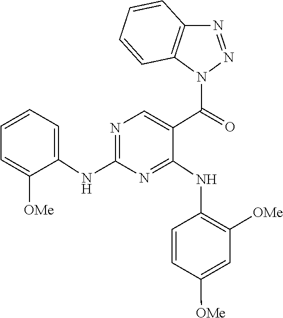 [SMILES: COc1ccc(Nc2nc(Nc3ccccc3OC)ncc2C(=O)n2nnc3ccccc32)c(OC)c1]

General procedure 4 was applied to 4-((2,4-dimethoxyphenyl)amino)-2-((2-methoxyphenyl)amino)pyrimidine-5-carboxylic acid (7.00 g, 17.7 mmol), EDCl (3.37 g, 17.7 mmol) and benzotriazole (2.10 g, 17.7 mmol) in dichloromethane (100 mL). The residue was purified by trituration with dichloromethane (30 mL). The precipitated solids were collected by filtration, washing with dichloromethane (3×10 mL) and dried under vacuum to yield the title compound as a yellow solid (3.00 g, 34%).

### Ethyl 2-cyano-3-(4-((2,4-dimethoxyphenyl)amino)-2-((2-methoxyphenyl)amino)pyrimidin-5-yl)-3-oxopropanoate

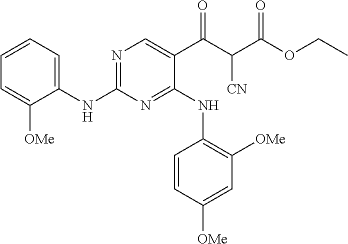 [SMILES: CCOC(=O)C(C#N)C(=O)c1cnc(Nc2ccccc2OC)nc1Nc1ccc(OC)cc1OC]

To a solution of ethyl 2-cyanoacetate (515 mg, 4.53 mmol, 1.5 eq.) in THF (20 mL) was added sodium hydride (241 mg, 6.04 mmol, 2.0 eq.) at 0° C. The mixture was stirred for 30 min then (1H-benzo[d][1,2,3]triazol-1-yl)(4-((2,4-dimethoxyphenyl)amino)-2-((2-methoxyphenyl)amino)pyrimidin-5-yl)methanone (1.50 g, 3.02 mmol, 1.0 eq.) in THF (30 mL) was added drop-wise. The resulting mixture was warmed to room temperature and stirred for 4 h. The reaction mixture was quenched with water (100 mL) and then acidified to pH 2 with 6N aqueous hydrochloric acid at 0° C. The resulting precipitate was collected by vacuum filtration, washing with water and 40-60 petroleum ether. The filter cake was dried under vacuum to yield the title compound as light yellow solid (1.00 g 67%).

### 8-(2,4-dimethoxyphenyl)-5-hydroxy-2-((2-methoxyphenyl)amino)-7-oxo-7,8-dihydropyrido[2,3-d]pyrimidine-6-carbonitrile

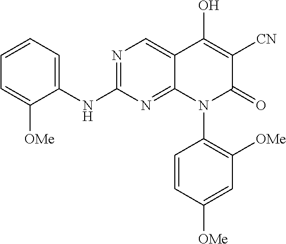 [SMILES: COc1ccc(-n2c(=O)c(C#N)c(O)c3cnc(Nc4ccccc4OC)nc32)c(OC)c1]

General procedure 6 was applied to ethyl 2-cyano-3-(4-((2,4-dimethoxyphenyl)amino)-2-((2-methoxyphenyl)amino)pyrimidin-5-yl)-3-oxopropanoate (1.00 g, 2.04 mmol), N,N-diisopropylethylamine (8.0 mL) and DBU (1.0 mL) to yield the title compound as a white solid (800 mg, 88%).

### 6-cyano-8-(2,4-dimethoxyphenyl)-2-((2-methoxyphenyl)amino)-7-oxo-7,8-dihydropyrido[2,3-d]pyrimidin-5-yl trifluoromethanesulfonate

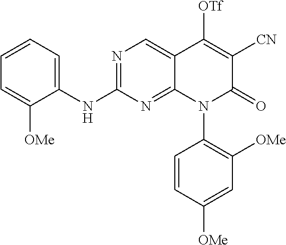 [SMILES: COc1ccc(-n2c(=O)c(C#N)c(OS(=O)(=O)C(F)(F)F)c3cnc(Nc4ccccc4OC)nc32)c(OC)c1]

General procedure was applied to 8-(2,4-dimetoxyphenyl)-5-hydroxy-2-((2-methoxyphenyl)amino)-7-oxo-7,8-dihydropyrido[2,3-d]pyrimidine-6-carbonitrile (200 mg, 0.449 mmol), triethylamine (113 mg, 1.12 mmol) and trifluoromethanesulfonic anhydride (190 mg, 0.674 mmol) in dichloromethane (5.0 mL). The residue was residue was purified by flash column chromatography eluting with ethyl acetate (50%) in 40-60 petroleum ether toy yield the title compound as a light yellow solid (130 mg, 50%).

### 8-(2,4-dimethoxyphenyl)-2-((2-methoxyphenyl)amino)-7-oxo-5-vinyl-7,8-dihydropyrido[2,3-d]pyrimidine-6-carbonitrile

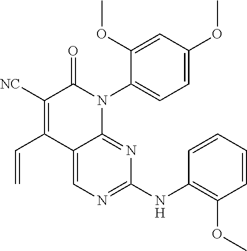 [SMILES: C=Cc1c(C#N)c(=O)n(-c2ccc(OC)cc2OC)c2nc(Nc3ccccc3OC)ncc12]

A solution of 6-cyano-8-(2,4-dimethoxyphenyl)-2-((2-methoxyphenyl)amino)-7-oxo-7,8-dihydropyrido[2,3-d]pyrimidin-5-yl trifluoromethanesulfonate (130 mg, 0.225 mmol, 1.0 eq.), 2-ethenyl-4,4,5,5-tetramethyl-1,3,2-dioxaborolane (52.0 mg, 0.338 mmol, 1.5 eq.), [1,1′-bis(diphenylphosphino)ferrocene]dichloropalladium(II) (16.3 mg, 22.0 μmol, 0.10 eq.) and potassium carbonate (62.2 mg, 0.450 mmol, 2.0 eq.) in dioxane (2.0 mL) and water (0.40 mL) was stirred for 1 h at 100° C. The resulting mixture was concentrated under reduced pressure then purified by reverse phase flash column chromatography eluting with acetonitrile (45-55%) in water (0.1% NH₄HCO₃) to yield the title compound as a light yellow solid (19.0 mg, 18%).

(ES, m/z): [M+H]⁺=456

¹H NMR (300 MHz, CDCl3-d₆) δ 8.82 (s, 1H), 8.23 (s, 1H), 7.60-7.56 (m, 1H), 7.26 (s, 1H), 7.15-7.11 (m, 2H), 7.06-6.98 (m, 1H), 6.98-6.96 (m, 1H), 6.72-6.68 (m, 2H), 6.57-6.54 (m, 1H), 3.93 (s, 3H), 3.88 (s, 2H), 3.64 (s, 2H).

### Example 161

### Ethyl 4-[2-(2-chlorophenyl)-N-methylacetamido]-2-(methylsulfanyl)pyrimidine-5-carboxylate

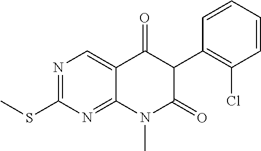 [SMILES: CSc1ncc2c(n1)N(C)C(=O)C(c1ccccc1Cl)C2=O]

General procedure 7 was applied to ethyl 4-(methylamino)-2-(methylsulfanyl)pyrimidine-5-carboxylate (8.00 g, 35.2 mmol), (2-chlorophenyl)acetyl chloride (6.65 g, 35.2 mmol) and sodium hydride (60% in oil, 850 mg) in DMF to yield the title compound as a yellow solid (5.00 g, 37%).

(ES, m/z): [M+H]⁺=334.0

### 6-(2-chlorophenyl)-8-methyl-2-(methylsulfanyl)-7-oxopyrido[2,3-d]pyrimidin-5-yl trifluoromethanesulfonate

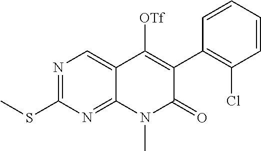 [SMILES: CSc1ncc2c(OS(=O)(=O)C(F)(F)F)c(-c3ccccc3Cl)c(=O)n(C)c2n1]

General procedure 8 was applied to 6-(2-chlorophenyl)-5-hydroxy-8-methyl-2-(methylsulfanyl)pyrido[2,3-d]pyrimidin-7-one (5.00 g, 15.0 mmol), triethylamine (3.03 g, 29.9 mmol) and triflic anhydride (4.23 g, 15.0 mmol) in dichloromethane. The residue was purified by reverse phase flash column chromatography eluting with acetonitrile (30-100%) in water to yield the title compound as a yellow oil (6.00 g, 86%).

(ES, m/z): [M+H]⁺=466.0

### 6-(2-chlorophenyl)-5-ethenyl-8-methyl-2-(methylsulfanyl)pyrido[2,3-d]pyrimidin-7-one

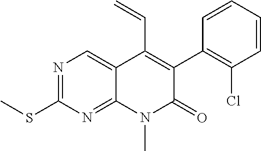 [SMILES: C=Cc1c(-c2ccccc2Cl)c(=O)n(C)c2nc(SC)ncc12]

To a solution of 6-(2-chlorophenyl)-8-methyl-2-(methylsulfanyl)-7-oxopyrido[2,3-d]pyrimidin-5-yl trifluoromethanesulfonate (700 mg, 1.50 mmol, 1.0 eq.) and tetrabutyl(ethenyl)stannane (562 mg, 1.50 mmol, 1.0 eq.) in DMF was added bis(triphenylphosphine)palladium(II) dichloride (105 mg, 0.150 mmol, 0.1 eq.). After stirring for 1 h at 100° C., the reaction was concentrated under reduced pressure. The residue was purified by flash column chromatography, eluting with ethyl acetate (10%) in 40-60 petroleum ether to afford the title compound as a yellow solid (440 mg, 85%).

(ES, m/z): [M+H]⁺=344.1

### 6-(2-chlorophenyl)-5-ethenyl-2-methanesulfonyl-8-methylpyrido[2,3-d]pyrimidin-7-one

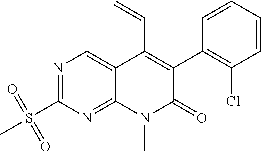 [SMILES: C=Cc1c(-c2ccccc2Cl)c(=O)n(C)c2nc(S(C)(=O)=O)ncc12]

General procedure 10 was applied to 6-(2-chlorophenyl)-5-ethenyl-8-methyl-2-(methylsulfanyl)pyrido[2,3-d]pyrimidin-7-one (440 mg, 1.28 mmol) and m-CPBA (662 mg, 3.84 mmol) in dichloromethane (10 mL). The residue was purified by flash column chromatography, eluting with ethyl acetate (20%) in 40-60 petroleum ether to afford the title compound as a yellow solid (480 mg, 100%).

(ES, m/z): [M+H]⁺=376.1

### 6-(2-chlorophenyl)-5-ethenyl-2-[(2-methoxyphenyl)amino]-8-methylpyrido[2,3-d]pyrimidin-7-one

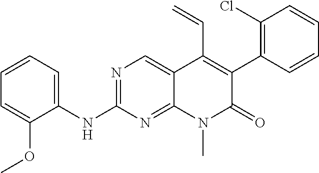 [SMILES: C=Cc1c(-c2ccccc2Cl)c(=O)n(C)c2nc(Nc3ccccc3OC)ncc12]

General procedure 13 was applied to 6-(2-chlorophenyl)-5-ethenyl-2-methanesulfonyl-8-methylpyrido[2,3-d]pyrimidin-7-one (150 mg, 0.399 mmol), trifluoroacetic acid (60 mg, 0.526 mmol) and o-anisidine (65.0 mg, 0.528 mmol) in 2-butanol (8.0 mL). The resulting mixture was stirred for 2 h at 100° C. The residue was purified by reverse phase flash column chromatography eluting with acetonitrile (10-50%) in water to yield the title compound as a light yellow solid (30.0 mg, 18%).

(ES, m/z): [M+H]⁺=419.0

¹H-NMR (300 MHz, DMSO-d6) δ8.84 (s, 1H), 8.79 (s, 1H), 8.08-8.05 (d, J=9, 1H), 7.55-7.41 (m, 2H), 7.41-7.28 (m, 2H), 7.28-7.26 (dd, J=9, 1H), 7.15-7.04 (s, dd, J=9, 1H), 7.04-7.02 (t, J=3, 1H), 6.59-6.49 (t, J=15, 1H), 5.60-5.59 (d, J=3, 1H), 5.56-5.55 (d, J=3, 1H), 3.87 (s, 2H) 3.59 (s, 2H), 1.24-0.78 (m, 1H).

### Example 162

### 5-ethenyl-8-methyl-2-(methylsulfanyl)-6-phenylpyrido[2,3-d]pyrimidin-7-one

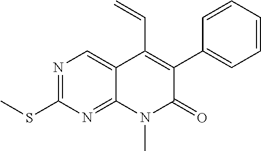 [SMILES: C=Cc1c(-c2ccccc2)c(=O)n(C)c2nc(SC)ncc12]

To a solution of 8-methyl-2-(methylsulfanyl)-7-oxo-6-phenylpyrido[2,3-d]pyrimidin-5-yl trifluoromethanesulfonate (500 mg, 1.16 mmol, 1.0 eq.) and 2-ethenyl-4,4,5,5-tetramethyl-1,3,2-dioxaborolane (357 mg, 2.32 mmol, 2.0 eq.) in dioxane (20 mL) and water (2.0 mL) was added potassium carbonate (16.0 mg, 0.116 mmol, 0.1 eq.) and (1,1′-bis(diphenylphosphino)ferrocene)palladium(II) dichloride (84.8 mg, 0.116 mmol, 0.1 eq.). After stirring for 1 h at 100° C., the resulting mixture was concentrated under reduced pressure. The residue was purified by flash column chromatography, eluting with ethyl acetate (25%) in 40-60 petroleum ether to afford the title compound as a yellow solid (250 mg, 69.72%).

(ES, m/z): [M+H]⁺=310.1

### 5-ethenyl-2-methanesulfonyl-8-methyl-6-phenylpyrido[2,3-d]pyrimidin-7-one

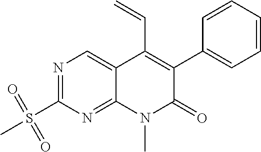 [SMILES: C=Cc1c(-c2ccccc2)c(=O)n(C)c2nc(S(C)(=O)=O)ncc12]

General procedure 10 was applied to 5-ethenyl-8-methyl-2-(methylsulfanyl)-6-phenylpyrido[2,3-d]pyrimidin-7-one (250 mg, 0.808 mmol) and m-CPBA (279 mg, 1.62 mmol.) in dichloromethane (10 mL) to yield the title compound as a yellow solid (200 mg, 72.5%).

(ES, m/z): [M+H]⁺=342.1

### 5-ethenyl-2-[(2-methoxyphenyl)amino]-8-methyl-6-phenylpyrido[2,3-d]pyrimidin-7-one

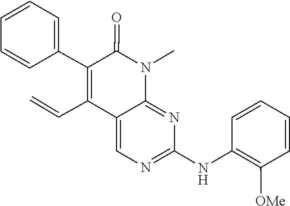 [SMILES: C=Cc1c(-c2ccccc2)c(=O)n(C)c2nc(Nc3ccccc3OC)ncc12]

General procedure 13 was applied to 5-ethenyl-2-methanesulfonyl-8-methyl-6-phenylpyrido[2,3-d]pyrimidin-7-one (100 mg, 0.293 mmol), o-anisidine (54.1 mg, 0.440 mmol) and trifluoroacetic acid (50.1 mg, 0.440 mmol) in 2-butanol (3.0 mL). The reaction was stirred overnight at 100° C. The crude product was purified by Prep-HPLC eluting with acetonitrile (50-68%) in water (0.1% NH₄HCO₃) to yield the title compound as a yellow solid (20.0 mg, 18%).

(ES, m/z): [M+H]⁺=385.2

¹H NMR (400 MHz, DMSO-d₆) δ 8.82 (s, 1H), 8.68 (s, 1H), 8.12-8.05 (m, 1H), 7.44-7.37 (m, 2H), 7.37-7.29 (m, 1H), 7.26-7.19 (m, 2H), 7.11 (dd, J=6.6, 1.8 Hz, 2H), 7.01 (ddd, J=8.8, 6.4, 2.4 Hz, 1H), 6.49 (dd, J=18.0, 11.8 Hz, 1H), 5.62 (dd, J=11.7, 1.3 Hz, 1H), 5.44 (dd, J=17.9, 1.4 Hz, 1H), 3.87 (s, 3H), 3.59 (s, 3H).

### Example 163

### 6-(2,6-dichlorophenyl)-5-ethenyl-8-methyl-2-(methylsulfanyl)pyrido[2,3-d]pyrimidin-7-one

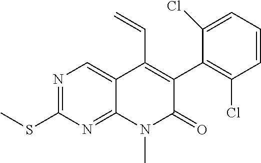 [SMILES: C=Cc1c(-c2c(Cl)cccc2Cl)c(=O)n(C)c2nc(SC)ncc12]

To a solution of 6-(2,6-dichlorophenyl)-8-methyl-2-(methylsulfanyl)-7-oxopyrido[2,3-d]pyrimidin-5-yl trifluoromethanesulfonate (1.00 g, 2.00 mmol, 1.0 eq.) and tributyl(ethenyl)stannane (761 mg, 2.40 mmol, 1.2 eq.) in DMF (10 mL) was added tetrakis(triphenylphosphine)palladium(0) (231 mg, 0.200 mmol, 0.1 eq.). After stirring for 1 h at 80° C., the resulting mixture was concentrated under reduced pressure. The residue was purified by flash column chromatography, eluting with ethyl acetate (25%) in 40-60 petroleum ether to afford the title compound as a yellow solid (600 mg, 79%).

(ES, m/z): [M+H]⁺=378.0

### 6-(2,6-dichlorophenyl)-5-ethenyl-2-methanesulfonyl-8-methylpyrido[2,3-d]pyrimidin-7-one

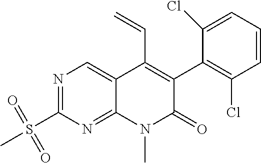 [SMILES: C=Cc1c(-c2c(Cl)cccc2Cl)c(=O)n(C)c2nc(S(C)(=O)=O)ncc12]

General procedure 10 was applied to 6-(2,6-dichlorophenyl)-5-ethenyl-8-methyl-2-(methylsulfanyl)pyrido[2,3-d]pyrimidin-7-one (600 mg, 1.58 mmol) and m-CPBA (547 mg, 3.17 mmol) in dichloromethane (10 mL) to yield the title compound as a yellow solid (500 mg, 77%).

(ES, m/z): [M+H]⁺=410.0

### 6-(2,6-dichlorophenyl)-5-ethenyl-2-[(2-methoxyphenyl)amino]-8-methylpyrido[2,3-]pyrimidin-7-one

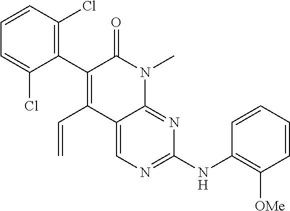 [SMILES: C=Cc1c(-c2c(Cl)cccc2Cl)c(=O)n(C)c2nc(Nc3ccccc3OC)ncc12]

General procedure 13 was applied to 6-(2,6-dichlorophenyl)-5-ethenyl-2-methansulfonyl-8-methylpyrido[2,3-d]pyrimidin-7-one (100 mg, 0.244 mmol), o-anisidine (45.0 mg, 0.366 mmol) and trifluoroacetic acid (41.7 mg, 0.366 mmol) in 2-butanol (3.0 mL). The reaction was stirred overnight at 100° C. then the crude product was purified by Prep-HPLC eluting with acetonitrile (55-85%) in water (0.1% NH₄HCO₃) to yield the title compound as a yellow solid (20.0 mg, 18%).

(ES, m/z): [M+H]⁺=453.1

¹H NMR (400 MHz, DMSO-d₆) δ 8.86 (d, J=7.1 Hz, 2H), 8.03 (d, J=7.9 Hz, 1H), 7.59-7.52 (m, 2H), 7.44 (dd, J=8.8, 7.3 Hz, 1H), 7.19-7.07 (m, 2H), 7.01 (td, J=7.4, 7.0, 1.8 Hz, 1H), 6.60 (dd, J=17.7, 11.7 Hz, 1H), 5.58 (dd, J=11.7, 1.3 Hz, 1H), 5.40 (dd, J=17.7, 1.3 Hz, 1H), 3.86 (s, 3H), 3.59 (s, 3H).

### Example 164

### 6-(2,6-dichlorophenyl)-5-ethenyl-2-{[3-(hydroxymethyl)phenyl]amino}-8-methylpyrido[2,3-d]pyrimidin-7-one

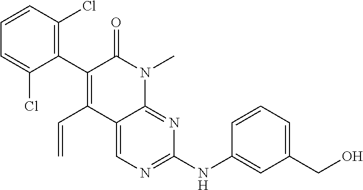 [SMILES: C=Cc1c(-c2c(Cl)cccc2Cl)c(=O)n(C)c2nc(Nc3cccc(CO)c3)ncc12]

General procedure 13 was applied to 6-(2,6-dichlorophenyl)-5-ethenyl-2-methanesulfonyl-8-methylpyrido[2,3-d]pyrimidin-7-one (100 mg, 0.244 mmol), (3-aminophenyl)methanol (45.0 mg, 0.366 mmol) and trifluoroacetic acid (41.7 mg, 0.366 mmol) in 2-butanol (3.0 mL). The reaction was stirred overnight at 100° C. The crude product was purified by Prep-HPLC eluting with acetonitrile (40-68%) in water to yield the title compound as a yellow solid (20.0 mg, 18%).

(ES, m/z): [M+H]⁺=453.1

¹H NMR (300 MHz, DMSO-d₆) δ 10.26 (s, 1H), 8.90 (s, 1H), 7.92 (s, 1H), 7.77-7.62 (m, 1H), 7.61-7.52 (m, 2H), 7.45 (dd, J=9.0, 7.1 Hz, 1H), 7.38-7.25 (m, 1H), 7.01 (d, J=7.5 Hz, 1H), 6.63 (dd, J=17.7, 11.7 Hz, 1H), 5.60 (dd, J=11.7, 1.3 Hz, 1H), 5.42 (dd, J=17.8, 1.3 Hz, 1H), 4.53 (s, 2H), 3.68 (s, 3H).

### Example 165

### 6-(2-chlorophenyl)-2-[(4-{[2-(dimethylamino)ethyl](methyl)amino}phenyl)amino]-5-ethenyl-8-methylpyrido[2,3-d]pyrimidin-7-one

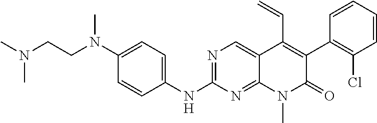 [SMILES: C=Cc1c(-c2ccccc2Cl)c(=O)n(C)c2nc(Nc3ccc(N(C)CCN(C)C)cc3)ncc12]

General procedure 13 was applied to 6-(2-chlorophenyl)-5-ethenyl-2-methanesulfonyl-8-methylpyrido[2,3-d]pyrimidin-7-one (90.0 mg, 0.239 mmol), trifluoroacetic acid (27.3 mg, 0.239 mmol) and N¹-[2-(dimethylamino)ethyl]-N¹-methylbenzene-1,4-diamine (46.3 mg, 0.239 mmol) in 2-butanol (2.0 mL). The resulting mixture was stirred for 1 h at 100° C. The residue was purified by reverse phase flash column chromatography eluting with acetonitrile (30-100%) in water to yield the title compound as a yellow solid (25.0 mg, 21%).

(ES, m/z): [M+H]⁺=489.3

¹H NMR (300 MHz, DMSO-d₆) δ 9.96 (s, 1H), 8.80 (s, 1H), 7.68 (d, J=8.4 Hz, 2H), 7.55-7.46 (m, 1H), 7.44-7.34 (m, 2H), 7.30-7.21 (m, 1H), 6.83 (d, J=9.0 Hz, 2H), 6.54 (dd, J=17.8, 11.7 Hz, 1H), 5.57 (dd, J=11.7, 1.4 Hz, 1H), 5.38 (dd, J=17.8, 1.4 Hz, 1H), 3.64 (s, 3H), 3.58 (d, J=7.6 Hz, 2H), 3.09 (s, 2H), 2.90 (s, 3H).

### Example 166

### 6-benzyl-5-ethenyl-8-methyl-2-(methylsulfanyl)pyrido[2,3-d]pyrimidin-7-one

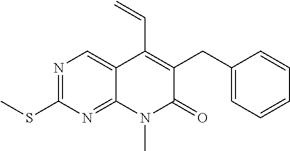 [SMILES: C=Cc1c(Cc2ccccc2)c(=O)n(C)c2nc(SC)ncc12]

To a solution of 6-benzyl-8-methyl-2-(methylsulfanyl)-7-oxopyrido[2,3-d]pyrimidin-5-yl trifluoromethanesulfonate (500 mg, 1.12 mmol, 1.0 eq.) and 2-ethenyl-4,4,5,5-tetramethyl-1,3,2-dioxaborolane (346 mg, 2.25 mmol, 2.0 eq.) in dioxane (20 mL) and water (2.0 mL) were added potassium carbonate (310 mg, 2.25 mmol, 2.0 eq.) and [1,1′-bis(diphenylphosphino)ferrocene]dichloropalladium(II) (84.8 mg, 0.116 mmol, 0.1 eq.). After stirring for 1 h at 100° C., the resulting mixture was concentrated under reduced pressure. The residue was purified by flash column chromatography, eluting with ethyl acetate (25%) in 40-60 petroleum ether to afford the title compound as a grey solid (300 mg, 82%).

(ES, m/z): [M+H]⁺=324.1

### 6-benzyl-5-ethenyl-2-methanesulfonyl-8-methylpyrido[2,3-d]pyrimidin-7-one

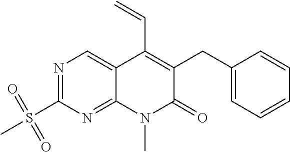 [SMILES: C=Cc1c(Cc2ccccc2)c(=O)n(C)c2nc(S(C)(=O)=O)ncc12]

General procedure 10 was applied to 6-benzyl-5-ethenyl-8-methyl-2-(methylsulfanyl)pyrido[2,3-d]pyrimidin-7-one (300 mg, 0.928 mmol) and m-CPBA (320 mg, 1.86 mmol) in dichloromethane (10 mL) to yield the title compound as a yellow solid (250 mg, 76%) which was used without further purification.

(ES, m/z): [M+H]⁺=356.1

### 6-benzyl-2-[(4-{[2-(dimethylamino)ethyl](methyl)amino}phenyl)amino]-5-ethenyl-8-methylpyrido[2,3-d]pyrimidin-7-one

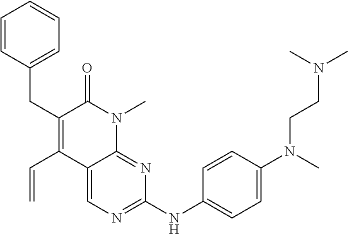 [SMILES: C=Cc1c(Cc2ccccc2)c(=O)n(C)c2nc(Nc3ccc(N(C)CCN(C)C)cc3)ncc12]

General procedure 13 was applied to 6-benzyl-5-ethenyl-2-methanesulfonyl-8-methylpyrido[2,3-d]pyrimidin-7-one (80.0 mg, 0.225 mmol), N¹-[2-(dimethylamino)ethyl]-N¹-methylbenzene-1,4-diamine (65.3 mg, 0.338 mmol) and trifluoroacetic acid (38.5 mg, 0.338 mmol) in 2-butanol (3.0 mL). The reaction mixture was stirred overnight at 100° C. The crude product was purified by Prep-HPLC eluting with acetonitrile (50-68%) in water (0.1% NH₄HCO₃) to yield the title compound as a yellow solid (30.0 mg, 28%).

(ES, m/z): [M+H]⁺=469.4

¹H NMR (300 MHz, DMSO-d₆) δ 9.77 (s, 1H), 8.68 (s, 1H), 7.60 (d, J=8.5 Hz, 2H), 7.29-7.07 (m, 5H), 6.97-6.84 (m, 1H), 6.70 (d, J=8.9 Hz, 2H), 5.82 (dd, J=11.7, 1.6 Hz, 1H), 5.53 (dd, J=17.9, 1.6 Hz, 1H), 3.93 (s, 2H), 3.61 (s, 3H), 3.40 (t, J=7.2 Hz, 2H), 2.88 (s, 3H), 2.40 (t, J=7.1 Hz, 2H), 2.20 (s, 6H).

### Example 167

### 6-(2,6-dichlorophenyl)-2-[(4-{[2-(dimethylamino)ethyl](methyl)amino}phenyl)amino]-5-ethenyl-8-methylpyrido[2,3-d]pyrimidin-7-one

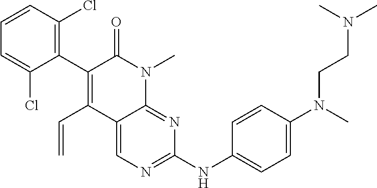 [SMILES: C=Cc1c(-c2c(Cl)cccc2Cl)c(=O)n(C)c2nc(Nc3ccc(N(C)CCN(C)C)cc3)ncc12]

General procedure 13 was applied to 6-(2,6-dichlorophenyl)-5-ethenyl-2-methanesulfonyl-8-methylpyrido[2,3-d]pyrimidin-7-one (100 mg, 0.244 mmol) and N¹-[2-(dimethylamino)ethyl]-N¹-methylbenzene-1,4-diamine (56.5 mg, 0.292 mmol), trifluoroacetic acid (41.7 mg, 0.366 mmol) in 2-butanol (3.0 mL). The reaction was stirred overnight at 100° C. The crude product was purified by Prep-HPLC eluting with acetonitrile (55-85%) in water (0.1% NH₄HCO₃) to yield the title compound as a red solid (20.0 mg, 20%).

(ES, m/z): [M+H]⁺=523.3

¹H NMR (300 MHz, DMSO-d₆) δ 9.97 (s, 1H), 8.81 (s, 1H), 7.74-7.50 (m, 4H), 7.49-7.39 (m, 1H), 6.73 (d, J=8.8 Hz, 2H), 6.65-6.550 (m, 1H), 5.62-5.52 (m, 1H), 5.45-5.350 (m, 1H), 3.64 (s, 3H), 3.43 (t, J=7.2 Hz, 2H), 2.90 (s, 3H), 2.27 (s, 6H).

### Example 168

### 6-(2-chlorophenyl)-5-ethenyl-2-[(3-methoxyphenyl)amino]-8-methylpyrido[2,3-d]pyrimidin-7-one

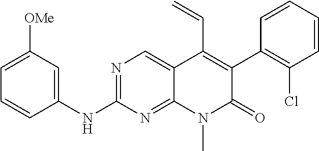 [SMILES: C=Cc1c(-c2ccccc2Cl)c(=O)n(C)c2nc(Nc3cccc(OC)c3)ncc12]

General procedure 13 was applied to 6-(2-chlorophenyl)-5-ethenyl-2-methanesulfonyl-8-methylpyrido[2,3-d]pyrimidin-7-one (60.0 mg, 0.160 mmol), m-anisidine (19.7 mg, 0.160 mmol) and trifluoroacetic acid (18.2 mg, 0.160 mmol) in 2-butanol. The resulting mixture was stirred for 1 h at 100° C. The residue was purified by reverse phase flash column chromatography eluting with acetonitrile (20-80%) in water to yield the title compound as an off-white solid (25.0 mg, 37%).

(ES, m/z): [M+H]⁺=419.3

¹H NMR (300 MHz, DMSO-d6) δ 10.20 (s, 1H), 8.89 (s, 1H), 7.65 (t, J=2.2 Hz, 1H), 7.58-7.50 (m, 1H), 7.45-7.32 (m, 3H), 7.32-7.20 (m, 2H), 6.66-6.50 (m, 2H), 5.59 (dd, J=11.7, 1.4 Hz, 1H), 5.40 (dd, J=17.8, 1.4 Hz, 1H), 3.79 (s, 3H), 3.68 (s, 3H).

### Example 169

### 6-(2-chlorophenyl)-5-ethenyl-2-{[3-(hydroxymethyl)phenyl]amino}-8-methylpyrido[2,3-]pyrimidin-7-one

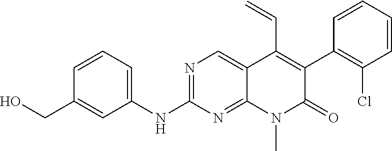 [SMILES: C=Cc1c(-c2ccccc2Cl)c(=O)n(C)c2nc(Nc3cccc(CO)c3)ncc12]

General procedure 13 was applied to 6-(2-chlorophenyl)-5-ethenyl-2-methanesulfonyl-8-methylpyrido[2,3-d]pyrimidin-7-one (60.0 mg, 0.160 mmol), (3-aminophenyl)methanol (19.7 mg, 0.160 mmol) and trifluoroacetic acid (18.2 mg, 0.160 mmol) in 2-butanol (2.0 mL). The resulting mixture was stirred for 1 h at 110° C. The residue was purified by reverse phase flash column chromatography eluting with acetonitrile (20-80%) in water to yield the title compound as an off-white solid (25.0 mg, 37%).

(ES, m/z): [M+H]⁺=419.2

¹H NMR (300 MHz, DMSO-d6) δ 10.20 (s, 1H), 8.88 (s, 1H), 7.92 (s, 1H), 7.74-7.63 (m, 1H), 7.53 (dt, J=7.9, 2.9 Hz, 1H), 7.45-7.35 (m, 2H), 7.34-7.22 (m, 2H), 7.00 (d, J=7.5 Hz, 1H), 6.56 (dd, J=17.8, 11.7 Hz, 1H), 5.58 (dd, J=11.7, 1.4 Hz, 1H), 5.40 (dd, J=17.8, 1.4 Hz, 1H), 5.21 (s, 1H), 4.52 (s, 2H), 3.67 (s, 3H).

### Example 170

### 6-(2-chlorophenyl)-5-ethenyl-2-{[2-methoxy-4-(4-methylpiperazin-1-yl)phenyl]amino}-8-methylpyrido[2,3-d]pyrimidin-7-one

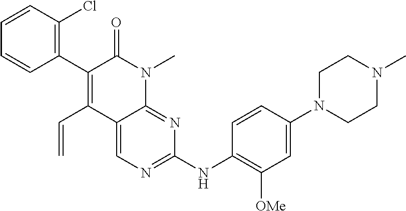 [SMILES: C=Cc1c(-c2ccccc2Cl)c(=O)n(C)c2nc(Nc3ccc(N4CCN(C)CC4)cc3OC)ncc12]

General procedure 13 was applied to 6-(2-chlorophenyl)-5-ethenyl-2-methanesulfonyl-8-methylpyrido[2,3-d]pyrimidin-7-one (80.0 mg, 0.213 mmol), trifluoroacetic acid (24.3 mg, 0.213 mmol) and 2-methoxy-4-(4-methylpiperazin-1-yl)aniline (47.1 mg, 0.213 mmol) in 2-butanol (2.0 mL). The resulting mixture was stirred for 1 h at 110° C. The residue was purified by reverse phase flash column chromatography eluting with acetonitrile (20-80%) in water to yield the title compound as a yellow green solid (25.0 mg, 23%).

(ES, m/z): [M+H]⁺=517.3

¹H NMR (300 MHz, DMSO-d6) δ 8.76 (s, 1H), 8.69 (s, 1H), 7.68 (s, 1H), 7.52 (dt, J=7.9, 2.9 Hz, 1H), 7.43-7.33 (m, 2H), 7.30-7.17 (m, 1H), 6.67 (d, J=2.5 Hz, 1H), 6.59-6.45 (m, 2H), 5.55 (dd, J=11.7, 1.4 Hz, 1H), 5.37 (dd, J=17.8, 1.5 Hz, 1H), 3.83 (s, 3H), 3.56 (s, 3H), 3.17 (t, J=5.0 Hz, 4H), 2.47 (s, 4H), 2.25 (s, 3H).

### Example 171

### 6-(2-chlorophenyl)-5-ethenyl-2-{[2-methoxy-4-(4-methylpiperazin-1-yl)phenyl]amino}-8-methylpyrido[2,3-d]pyrimidin-7-one

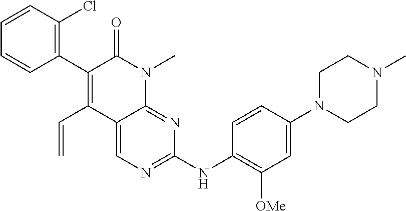 [SMILES: C=Cc1c(-c2ccccc2Cl)c(=O)n(C)c2nc(Nc3ccc(N4CCN(C)CC4)cc3OC)ncc12]

General procedure 13 was applied to 6-(2-chlorophenyl)-5-ethenyl-2-methanesulfonyl-8-methylpyrido[2,3-d]pyrimidin-7-one (80.0 mg, 0.213 mmol), 2-methoxy-4-(4-methylpiperazin-1-yl)aniline (47.1 mg, 0.213 mmol) and trifluoroacetic acid (24.3 mg, 0.213 mmol) in 2-butanol (2.0 mL). The resulting mixture was stirred for 1 h at 110° C. The residue was purified by reverse phase flash column chromatography eluting with acetonitrile (20-80%) in water to yield the title compound as a yellow green solid (25.0 mg, 23%).

(ES, m/z): [M+H]⁺=517.3

¹H NMR (300 MHz, DMSO-d6) δ 8.76 (s, 1H), 8.69 (s, 1H), 7.68 (s, 1H), 7.52 (dt, J=7.9, 2.9 Hz, 1H), 7.43-7.33 (m, 2H), 7.30-7.17 (m, 1H), 6.67 (d, J=2.5 Hz, 1H), 6.59-6.45 (m, 2H), 5.55 (dd, J=11.7, 1.4 Hz, 1H), 5.37 (dd, J=17.8, 1.5 Hz, 1H), 3.83 (s, 3H), 3.56 (s, 3H), 3.17 (t, J=5.0 Hz, 4H), 2.47 (s, 4H), 2.25 (s, 3H).

### Example 172

### 6-(2-chlorophenyl)-2-({4-[2-(dimethylamino)ethoxy]phenyl}amino)-5-ethenyl-8-methylpyrido[2,3-d]pyrimidin-7-one

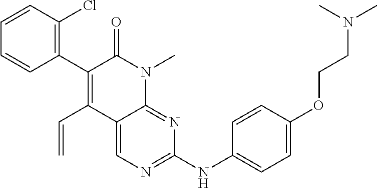 [SMILES: C=Cc1c(-c2ccccc2Cl)c(=O)n(C)c2nc(Nc3ccc(OCCN(C)C)cc3)ncc12]

General procedure 13 was applied to 6-(2-chlorophenyl)-5-ethenyl-2-methanesulfonyl-8-methylpyrido[2,3-d]pyrimidin-7-one (80.0 mg, 0.213 mmol), trifluoroacetic acid (24.27 mg, 0.213 mmol) and 4-[2-(dimethylamino)ethoxy]aniline (38.4 mg, 0.213 mmol) in 2-butanol (2.0 mL). The resulting mixture was stirred for 1 h at 100° C. The residue was purified by reverse phase flash column chromatography eluting with acetonitrile (20-80%) in water to yield the title compound as a yellow solid (35.0 mg, 35%).

(ES, m/z): [M+H]⁺=476.3

¹H NMR (300 MHz, DMSO-d6) δ 10.05 (s, 1H), 8.83 (s, 1H), 7.72 (d, J=8.7 Hz, 2H), 7.59-7.49 (m, 1H), 7.44-7.35 (m, 2H), 7.30-7.23 (m, 1H), 7.00-6.91 (m, 2H), 6.54 (dd, J=17.8, 11.7 Hz, 1H), 5.57 (dd, J=11.7, 1.4 Hz, 1H), 5.39 (dd, J=17.8, 1.4 Hz, 1H), 4.05 (t, J=5.8 Hz, 2H), 3.64 (s, 3H), 2.63 (t, J=5.8 Hz, 2H), 2.23 (s, 6H).

### Example 173

### 2-amino-6-(2-chlorophenyl)-5-ethenyl-8-methylpyrido[2,3-d]pyrimidin-7-one

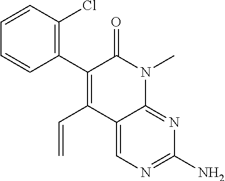 [SMILES: C=Cc1c(-c2ccccc2Cl)c(=O)n(C)c2nc(N)ncc12]

General procedure 13 was applied to 6-(2-chlorophenyl)-5-ethenyl-2-methanesulfonyl-8-methylpyrido[2,3-d]pyrimidin-7-one (80.0 mg, 0.213 mmol), trifluoroacetic acid (72.8 mg, 0.639 mmol) and 4-methoxybenzenemethanamine, (29.2 mg, 0.213 mmol) in 2-butanol. The resulting mixture was stirred for 1 h at 110° C. The residue was purified by reverse phase flash column chromatography eluting with acetonitrile (20-80%) in water to yield the title compound as a white solid.

(ES, m/z): [M+H]⁺=313.1

¹H NMR (300 MHz, DMSO-d6) δ 8.68 (s, 1H), 7.55-7.46 (m, 1H), 7.45-7.28 (m, 4H), 7.26-7.16 (m, 1H), 6.50 (dd, J=17.8, 11.7 Hz, 1H), 5.53 (dd, J=11.7, 1.5 Hz, 1H), 5.34 (dd, J=17.8, 1.5 Hz, 1H), 3.52 (s, 3H).

### Example 174

### 6-(2-chlorophenyl)-8-methyl-2-((4-(4-methylpiperazin-1-yl)butyl)amino)-5-vinylpyrido[2,3-d]pyrimidin-7(8H)-one

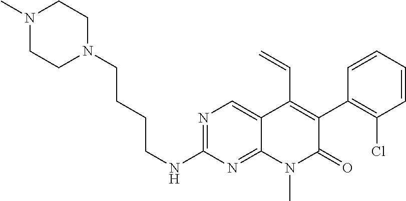 [SMILES: C=Cc1c(-c2ccccc2Cl)c(=O)n(C)c2nc(NCCCCN3CCN(C)CC3)ncc12]

General procedure 13 was applied to 6-(2-chlorophenyl)-5-ethenyl-2-methanesulfonyl-8-methylpyrido[2,3-d]pyrimidin-7-one (80.0 mg, 0.213 mmol), trifluoroacetic acid (24.3 mg, 0.213 mmol) and 4-(4-methylpiperazin-1-yl)butan-1-amine (36.4 mg, 0.213 mmol) in 2-butanol (2.0 mL). The resulting mixture was stirred for 1 h at 100° C. The residue was purified by reverse phase flash column chromatography eluting with acetonitrile (10-50%) in water to yield the title compound.

(ES, m/z): [M+H]⁺=467.3

¹H NMR (300 MHz, DMSO-d6) δ 8.70 (d, J=21.0 Hz, 1H), 8.07-7.87 (m, 1H), 7.56-7.46 (m, 1H), 7.44-7.33 (m, 2H), 7.28-7.18 (m, 1H), 6.48 (dd, J=17.8, 11.7 Hz, 1H), 5.53 (dd, J=11.7, 1.4 Hz, 1H), 5.34 (dd, J=17.8, 1.5 Hz, 1H), 3.54 (s, 3H), 3.41 (s, 3H), 2.45-2.22 (m, 9H), 2.13 (s, 3H), 1.57 (dt, J=32.7, 7.3 Hz, 4H).

### Example 175

### 8-(Isoxazol-4-ylmethyl)-6-methyl-2-(methylthio)-5-((triisopropylsilyl)ethynyl)pyrido[2,3-d]pyrimidin-7(8H)-one

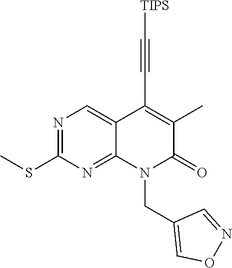

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), 1,2-oxazol-4-ylmethanol (280 mg, 2.84 mmol), triphenylphosphine (1.35 g, 5.16 mmol) and DIAD (1.04 g, 5.16 mmol) in dichloromethane (15 mL). The residue was purified by flash column chromatography, eluting with ethyl acetate (10%) in 40-60 petroleum ether to afford the title compound as a colourless oil (685 mg, 56%).

### 2-Methanesulfonyl-6-methvl-8-(1,2-oxazol-4-ylmethyl)-5-[2-(triisopropylsilyl)ethynyl]pyrido[2,3-d]pyrimidin-7-one

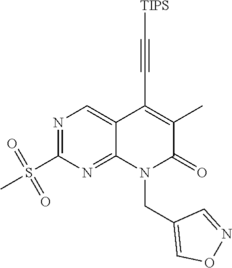

General procedure 10 was applied to 6-methyl-2-(methylsulfanyl)-8-(1,2-oxazol-4-ylmethyl)-5-[2-(triisopropylsilyl)ethynyl]pyrido[2,3-d]pyrimidin-7-one (670 mg, 1.43 mmol) and mCPBA (542 mg, 3.14 mmol) in dichloromethane (10 mL) to yield the title compound as a light yellow oil (668 mg, 93%).

### 6-Methyl-2-{[4-(4-methylpiperazin-1-yl)phenyl]amino}-8-(1,2-oxazol-4-ylmethyl)-5-[2-(triisopropylsilyl)ethynyl]pyrido[2,3-d]pyrimidin-7-one

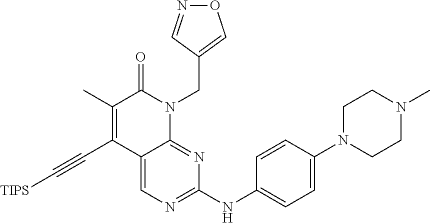

General procedure 13 was applied to 2-methanesulfonyl-6-methyl-8-(1,2-oxazol-4-ylmethyl)-5-[2-(triisopropylsilyl)ethynyl]pyrido[2,3-d]pyrimidin-7-one (600 mg, 1.20 mmol), 4-(4-methylpiperazin-1-yl)aniline (275 mg, 1.44 mmol) and trifluoroacetic acid (0.180 mL, 2.39 mmol) in dioxane (8.0 mL), stirred overnight at 100° C. The residue was purified by reverse phase flash column chromatography eluting with acetonitrile in water (0.1% formic acid), to yield the title compound as a yellow solid (220 mg, 30%).

### 5-Ethynyl-6-methyl-2-{[4-(4-methylpiperazin-1-yl)phenyl]aminol}-8-(1,2-oxazol-4-ylmethyl)pyrido[2,3-d]pyrimidin-7-one

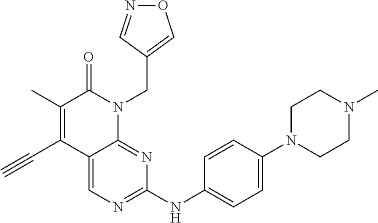 [SMILES: C#Cc1c(C)c(=O)n(Cc2cnoc2)c2nc(Nc3ccc(N4CCN(C)CC4)cc3)ncc12]

General procedure 15 was applied to 6-methyl-2-{[4-(4-methylpiperazin-1-yl)phenyl]amino}-8-(1,2-oxazol-4-ylmethyl)-5-[2-(triisopropylsilyl)ethynyl]pyrido[2,3-d]pyrimidin-7-one (109 mg, 0.178 mmol) and potassium fluoride (103 mg, 1.78 mmol) in THF (2.0 mL), DMF (2.0 mL) and water (0.50 mL), stirred for 1 hour at 60° C. The residue was purified by reverse phase flash column chromatography eluting with acetonitrile (10-50%) in water (0.1% formic acid), to yield the title compound as a yellow solid (51.0 mg, 63%).

(ES, m/z): [M+H]⁺=456.15

¹H-NMR (400 MHz, DMSO-d₆) δ 9.93 (s, 1H), 8.75 (s, 2H), 8.49 (s, 1H), 7.59-7.49 (m, 2H), 6.97-6.87 (m, 2H), 5.29 (s, 2H), 5.22 (s, 1H), 3.09 (t, J=5.0 Hz, 4H), 2.46 (t, J=5.0 Hz, 4H), 2.23 (d, J=3.3 Hz, 6H).

### Example 176

### 6-Methyl-2-(methylsulfanyl)-8-(1,3-oxazol-5-ylmethyl)-5-[2-(triisopropylsilyl)ethynyl]pyrido[2,3-d]pyrimidin-7-one

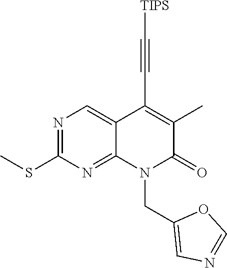

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), 1,3-oxazol-5-ylmethanol (280 mg, 2.84 mmol), triphenylphosphine (1.35 g, 5.16 mmol) and DIAD (1.04 g, 5.16 mmol) in dichloromethane (15 mL). The residue was purified by flash column chromatography, eluting with ethyl acetate (5%) in 40-60 petroleum ether to afford the title compound as a yellow solid (1.10 g, 91%).

### 2-Methanesulfonyl-6-methyl-8-(1,3-oxazol-5-ylmethyl)-5-[2-(triisopropylsilyl)ethynyl]pyrido[2,3-d]pyrimidin-7-one

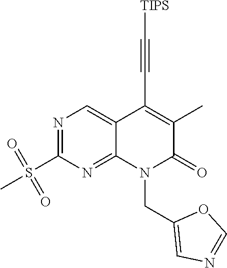

General procedure 10 was applied to 6-methyl-2-(methylsulfanyl)-8-(1,3-oxazol-5-ylmethyl)-5-[2-(triisopropylsilyl)ethynyl]pyrido[2,3-d]pyrimidin-7-one (520 mg, 1.11 mmol) and m-CPBA (421 mg, 2.44 mmol) in dichloromethane (8.0 mL) to yield the title compound as a light yellow oil (512 mg, 92%).

### 6-Methyl-2-{[4-(4-methylpiperazin-1-yl)phenyl]aminol}-8-(1,3-oxazol-5-ylmethyl)-5-[2-(triisopropylsilyl)ethynyl]pyrido[2,3-d]pyrimidin-7-one

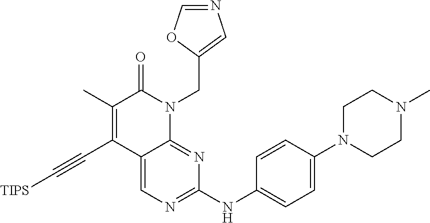

General procedure 13 was applied to 2-methanesulfonyl-6-methyl-8-(1,3-oxazol-5-ylmethyl)-5-[2-(triisopropylsilyl)ethynyl]pyrido[2,3-d]pyrimidin-7-one (550 mg, 1.09 mmol), 4-(4-methylpiperazin-1-yl)aniline (273 mg, 1.43 mmol) and trifluoroacetic acid (0.160 mL, 2.19 mmol) in dioxane (8.0 mL), stirred overnight at 100° C. The residue was purified by reverse phase flash column chromatography eluting with acetonitrile (10-80%) in water (0.1% formic acid), to yield the title compound as a yellow solid (109 mg, 16%).

### 5-Ethynyl-6-methvl-2-{[4-(4-methylpiperazin-1-yl)phenyl]aminol}-8-(1,3-oxazol-5-ylmethyl)pyrido[2,3-d]pyrimidin-7-one

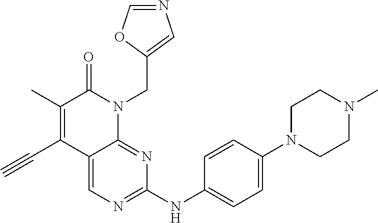 [SMILES: C#Cc1c(C)c(=O)n(Cc2cnco2)c2nc(Nc3ccc(N4CCN(C)CC4)cc3)ncc12]

General procedure 15 was applied to 6-methyl-2-{[4-(4-methylpiperazin-1-yl)phenyl]amino}-8-(1,3-oxazol-5-ylmethyl)-5-[2-(triisopropylsilyl)ethynyl]pyrido[2,3-d]pyrimidin-7-one (80.0 mg, 0.131 mmol) and potassium fluoride (75.9 mg, 1.31 mmol) in THF (2.0 mL), DMF (2.0 mL) and waster (0.50 mL), stirred for 1 hour at 60° C. The residue was purified by reverse phase flash column chromatography eluting with acetonitrile (10-50%) in water (0.1% formic acid), to yield the title compound as a yellow solid (13.3 mg, 22%).

(ES, m/z): [M+H]⁺=456.15

¹H-NMR (400 MHz, DMSO-d₆) δ 9.97 (s, 1H), 8.76 (s, 1H), 8.21 (d, J=39.0 Hz, 2H), 7.63-7.51 (m, 2H), 7.04-6.85 (m, 3H), 5.51 (s, 2H), 5.24 (s, 1H), 3.10 (t, J=5.0 Hz, 4H), 2.46 (t, J=5.0 Hz, 4H), 2.23 (d, J=3.0 Hz, 6H).

### Example 177

### tert-Butyl 2-{[6-methyl-2-(methylsulfanyl)-7-oxo-5-[2-(triisopropylsilyl)ethynyl]pyrido[2,3-d]pyrimidin-8-yl]methyl}imidazole-1-carboxylate

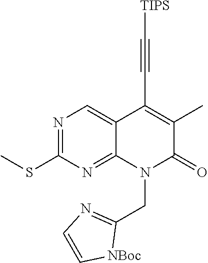

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), tert-butyl 2-(hydroxymethyl)imidazole-1-carboxylate (560 mg, 2.84 mmol), triphenylphosphine (1.35 g, 5.16 mmol) and DIAD (1.04 g, 5.16 mmol) in dichloromethane (15 mL). The residue was purified by flash column chromatography, eluting with ethyl acetate (10%) in 40-60 petroleum ether to afford the title compound as a white solid (584 mg, 40%).

### tert-Butyl 2-({2-methanesulfonyl-6-methyl-7-oxo-5-[2-(triisopropylsilyl)ethynyl]pyrido[2,3-d]pyrimidin-8-yl}methyl)imidazole-1-carboxylate

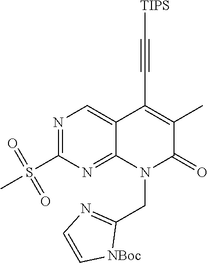

General procedure 10 was applied to tert-butyl 2-{[6-methyl-2-(methylsulfanyl)-7-oxo-5-[2-(triisopropylsilyl)ethynyl]pyrido[2,3-d]pyrimidin-8-yl]methyl}imidazole-1-carboxylate (670 mg, 1.18 mmol) and mCPBA (448 mg, 2.59 mmol) in dichloromethane (10 mL). The residue was purified by flash column chromatography, eluting with ethyl acetate (10%) in 40-60 petroleum ether to afford the title compound as a light yellow oil (675 mg, 95%).

### 8-(1H-Imidazol-2-ylmethyl)-6-methyl-2-{[4-(4-methylpiperazin-1-yl)phenyl]amino}-5-[2-(triisopropylsilyl)ethynyl]pyrido[2,3-d]pyrimidin-7-one

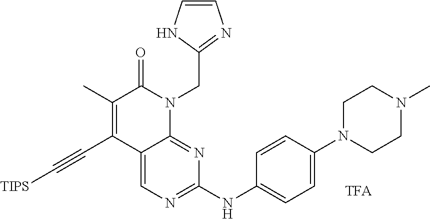

General procedure 13 was applied to tert-butyl 2-({2-methanesulfonyl-6-methyl-7-oxo-5-[2-(triisopropylsilyl)ethynyl]pyrido[2,3-d]pyrimidin-8-yl}methyl)imidazole-1-carboxylate (650 mg, 1.08 mmol), 4-(4-methylpiperazin-1-yl)aniline (269 mg, 1.41 mmol) and trifluoroacetic acid (0.160 mL, 2.17 mmol) in dioxane (8.0 mL), stirred overnight at 100° C.

The residue was purified by reverse phase flash column chromatography eluting with acetonitrile (10-80%) in water (0.1% formic acid), to yield the title compound as a light yellow solid (156 mg, 23%).

### 5-Ethynyl-8-(1H-imidazol-2-ylmethyl)-6-methyl-2-{[4-(4-methylpiperazin-1-yl)phenyl]amino}pyrido[2,3-d]pyrimidin-7-one

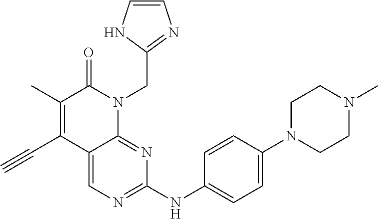 [SMILES: C#Cc1c(C)c(=O)n(Cc2ncc[nH]2)c2nc(Nc3ccc(N4CCN(C)CC4)cc3)ncc12]

General procedure 15 was applied to 8-(1H-imidazol-2-ylmethyl)-6-methyl-2-{[4-(4-methylpiperazin-1-yl)phenyl]amino}-5-[2-(triisopropylsilyl)ethynyl]pyrido[2,3-d]pyrimidin-7-one (150 mg, 0.246 mmol) and potassium fluoride (142 mg, 2.46 mmol) in THF (2.0 mL), DMF (2.0 mL) and water (0.50 mL), stirred for 1 hour at 60° C. The residue was purified by reverse phase flash column chromatography eluting with acetonitrile (10-50%) in water (0.1% formic acid), to yield the title compound as a yellow solid (44.0 mg, 39%).

(ES, m/z): [M+H]⁺=455.15

¹H-NMR (400 MHz, DMSO-d₆) δ 9.90 (s, 1H), 8.77 (s, 1H), 8.14 (s, 1H), 7.40 (s, 2H), 6.97-6.73 (m, 4H), 5.47 (s, 2H), 5.24 (s, 1H), 3.09 (t, J=5.0 Hz, 4H), 2.56 (t, J=5.0 Hz, 4H), 2.27 (d, J=22.2 Hz, 6H).

### Example 178

### 6-Methyl-2-(methylsulfanyl)-8-(pyridin-2-ylmethyl)-5-[2-(triisopropylsilyl)ethynyl]pyrido[2,3-d]pyrimidin-7-one

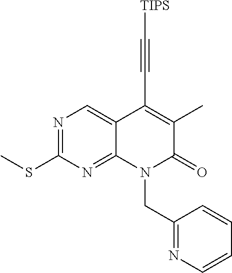

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), 2-pyridinemethanol (310 mg, 2.84 mmol), triphenylphosphine (1.35 g, 5.16 mmol) and DIAD (1.04 g, 5.16 mmol) in dichloromethane (10 mL). The residue was purified by flash column chromatography, eluting with ethyl acetate (5%) in 40-60 petroleum ether to afford the title compound as a light yellow solid (1.02 g, 82%).

### 2-Methanesulfonyl-6-methyl-8-(pyridin-2-ylmethyl)-5-[2-(triisopropylsilyl)ethynyl]pyrido[2,3-d]pyrimidin-7-one

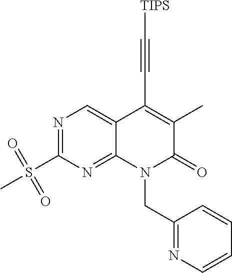

General procedure 10 was applied to 6-methyl-2-(methylsulfanyl)-8-(pyridin-2-ylmethyl)-5-[2-(triisopropylsilyl)ethynyl]pyrido[2,3-d]pyrimidin-7-one (372 mg, 0.777 mmol) and mCPBA (134 mg, 0.777 mmol) in dichloromethane (8.0 mL) to yield the title compound as a light yellow solid (352 mg, 89%).

### 6-Methyl-2-{[4-(4-methylpiperazin-1-yl)phenyl]aminol}-8-(Pyridin-2-ylmethyl)-5-[2-(triisopropylsilyl)ethynyl]pyrido[2,3-d]pyrimidin-7-one

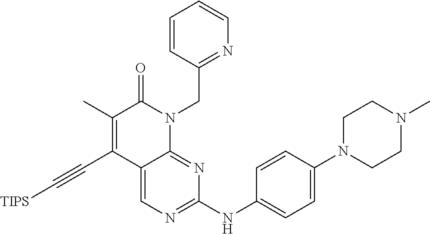

General procedure 13 was applied to 2-methanesulfonyl-6-methyl-8-(pyridin-2-ylmethyl)-5-[2-(triisopropylsilyl)ethynyl]pyrido[2,3-d]pyrimidin-7-one (630 mg, 1.23 mmol), 4-(4-methylpiperazin-1-yl)aniline (307 mg, 1.60 mmol) and trifluoroacetic acid (0.180 mL, 2.47 mmol) in dioxane (8.0 mL), stirred overnight at 100° C. The residue was purified by reverse phase flash column chromatography eluting with acetonitrile (10-80%) in water (0.1% formic acid), to yield the title compound as a light yellow solid (150 mg, 19%).

### 5-Ethynyl-6-methyl-2-{[4-(4-methylpiperazin-1-yl)phenyl]amino}-8-(pyridin-2-ylmethyl)pyrido[2,3-d]pyrimidin-7-one

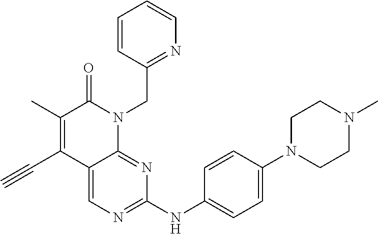 [SMILES: C#Cc1c(C)c(=O)n(Cc2ccccn2)c2nc(Nc3ccc(N4CCN(C)CC4)cc3)ncc12]

General procedure 15 was applied to 6-methyl-2-{[4-(4-methylpiperazin-1-yl)phenyl]amino}-8-(pyridin-2-ylmethyl)-5-[2-(triisopropylsilyl)ethynyl]pyrido[2,3-d]pyrimidin-7-one (120 mg, 0.193 mmol) and potassium fluoride (112 mg, 1.93 mmol) in THF (2.0 mL), DMF (2.0 mL) and water (0.50 mL) was stirred for 1 hour at 60° C. The residue was purified by reverse phase flash column chromatography eluting with acetonitrile (10-50%) in water (0.1% formic acid), to yield the title compound as a yellow solid (30.0 mg, 33%).

(ES, m/z): [M+H]⁺=466.15

¹H-NMR (400 MHz, DMSO-d₆) δ 9.87 (s, 1H), 8.77 (s, 1H), 8.49-8.40 (m, 1H), 7.74 (td, J=7.7, 1.8 Hz, 1H), 7.41-7.18 (m, 4H), 6.74 (d, J=8.4 Hz, 2H), 5.59 (s, 2H), 5.26 (s, 1H), 3.05 (t, J=4.9 Hz, 4H), 2.45 (t, J=5.0 Hz, 4H), 2.24 (d, J=12.0 Hz, 6H).

### Example 179

### 6-Methyl-2-(methylsulfanyl)-8-(pyridin-4-ylmethyl)-5-[2-(triisopropylsilyl)ethynyl]pyrido[2,3-d]pyrimidin-7-one

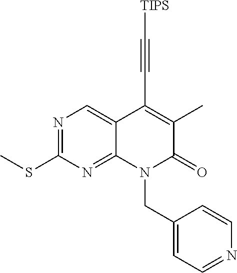

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), 4-pyridinemethanol (310 mg, 2.84 mmol), triphenylphosphine (1.35 g, 5.16 mmol) and DIAD (1.04 g, 5.16 mmol) in dichloromethane (15 mL). The residue was purified by flash column chromatography, eluting with ethyl acetate (10%) in 40-60 petroleum ether to afford the title compound as a light yellow oil (685 mg, 55%).

### 2-Methanesulfonyl-6-methyl-8-(pyridin-4-ylmethyl)-5-[2-(triisopropylsilyl)ethynyl]pyrido[2,3-d]pyrimidin-7-one

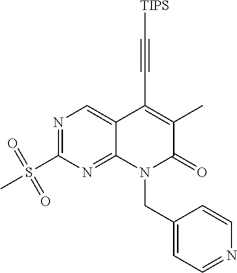

General procedure 10 was applied to 6-methyl-2-(methylsulfanyl)-8-(pyridin-4-ylmethyl)-5-[2-(triisopropylsilyl)ethynyl]pyrido[2,3-d]pyrimidin-7-one (650 mg, 1.36 mmol) and mCPBA (246 mg, 1.42 mmol) in dichloromethane (10 mL) to yield the title compound as a light yellow solid (628 mg, 90%).

### 6-Methyl-2-{[4-(4-methylpiperazin-1-yl)phenyl]amino}-8-(pyridin-4-ylmethyl)-5-[2-(triisopropylsilyl)ethynyl]pyrido[2,3-d]pyrimidin-7-one

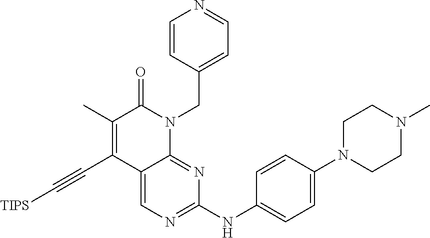

General procedure 13 was applied to 2-methanesulfonyl-6-methyl-8-(pyridin-4-ylmethyl)-5-[2-(triisopropylsilyl)ethynyl]pyrido[2,3-d]pyrimidin-7-one (620 mg, 1.21 mmol), 4-(4-methylpiperazin-1-yl)aniline (302 mg, 1.58 mmol) and trifluoroacetic acid (0.180 mL, 2.43 mmol) in dioxane (10 mL), stirred overnight at 100° C. The residue was purified by reverse phase flash column chromatography eluting with acetonitrile (10-80%) in water (0.1% formic acid), to yield the title compound as a light yellow solid (151 mg, 20%).

### 5-Ethynyl-6-methyl-2-{[4-(4-methylpiperazin-1-yl)phenyl]amino}-8-(pyridin-4-ylmethyl)pyrido[2,3-d]pyrimidin-7-one

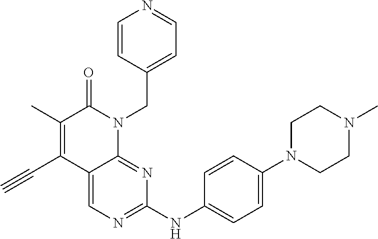 [SMILES: C#Cc1c(C)c(=O)n(Cc2ccncc2)c2nc(Nc3ccc(N4CCN(C)CC4)cc3)ncc12]

General procedure 15 was applied to 6-methyl-2-{[4-(4-methylpiperazin-1-yl)phenyl]amino}-8-(pyridin-4-ylmethyl)-5-[2-(triisopropylsilyl)ethynyl]pyrido[2,3-d]pyrimidin-7-one (150 mg, 0.241 mmol) and potassium fluoride (140 mg, 2.41 mmol) in THF (2.0 mL), DMF (2.0 mL) and water (0.50 mL), stirred for 1 hour at 60° C. The residue was purified by reverse phase flash column chromatography eluting with acetonitrile (10-50%) in water (0.1% formic acid), to yield the title compound as a brown solid (9.50 mg, 8%).

(ES, m/z): [M+H]⁺=466.15

¹H-NMR (400 MHz, DMSO-d₆) δ 9.92 (s, 1H), 8.79 (s, 1H), 8.49-8.44 (m, 2H), 7.35 (d, J=8.3 Hz, 2H), 7.20 (d, J=5.4 Hz, 2H), 6.80 (d, J=8.6 Hz, 2H), 5.48 (s, 2H), 5.28 (s, 1H), 3.07 (t, J=5.0 Hz, 4H), 2.46 (t, J=5.0 Hz, 4H), 2.27 (s, 3H), 2.23 (s, 3H).

### Example 180

### 6-Methyl-8-[(1-methylpyrazol-4-yl)methyl]-2-(methylsulfanyl)-5-[2-(triisopropylsilyl)ethynyl]pyrido[2,3-d]pyrimidin-7-one

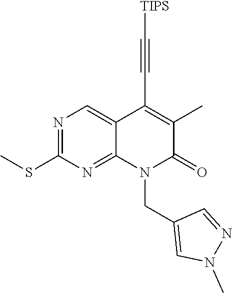

General procedure 18 was applied to 6-methyl-2-(methylsulfanyl)-5-[2-(triisopropylsilyl)ethynyl]-8H-pyrido[2,3-d]pyrimidin-7-one (1.00 g, 2.58 mmol), (1-methylpyrazol-4-yl)methanol (320 mg, 2.84 mmol), triphenylphosphine (1.35 g, 5.16 mmol) and DIAD (1.04 g, 5.16 mmol) in dichloromethane (15 mL). The residue was purified by flash column chromatography, eluting with ethyl acetate (10%) in 40-60 petroleum ether to afford the title compound as a light yellow oil (652 mg, 52%).

### 2-Methanesulfonyl-6-methvl-8-[(1-methylpyrazol-4-yl)methyl]-5-[2-(triisopropylsilyl)ethynyl]pyrido[2,3-d]pyrimidin-7-one

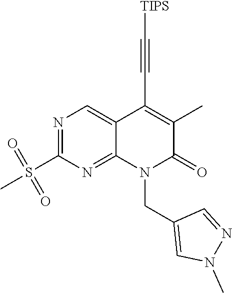

General procedure 10 was applied to 6-methyl-8-[(1-methylpyrazol-4-yl)methyl]-2-(methylsulfanyl)-5-[2-(triisopropylsilyl)ethynyl]pyrido[2,3-d]pyrimidin-7-one (650 mg, 1.35 mmol) and mCPBA (512 mg, 2.97 mmol) to yield the title compound as a light yellow solid (648 mg, 93%).

### 6-Methyl-2-{[4-(4-methylpiperazin-1-yl)phenyl]amino}-8-[(1-methylpyrazol-4-yl)methyl]-5-[2-(triisopropylsilyl)ethynyl]pyrido[2,3-d]pyrimidin-7-one

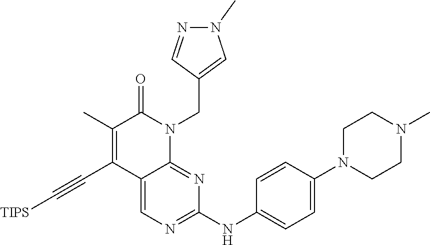

General procedure 13 was applied to 2-methanesulfonyl-6-methyl-8-[(1-methylpyrazol-4-yl)methyl]-5-[2-(triisopropylsilyl)ethynyl]pyrido[2,3-d]pyrimidin-7-one (600 mg, 1.17 mmol), 4-(4-methylpiperazin-1-yl)aniline (290 mg, 1.52 mmol) and trifluoroacetic acid (0.170 mL, 2.34 mmol) in dioxane (10 mL), stirred overnight at 100° C. The residue was purified by reverse phase flash column chromatography eluting with acetonitrile (10-80%) in water (0.1% formic acid), to yield the title compound as a yellow solid (92.0 mg, 12%).

### 5-Ethynyl-6-methyl-2-{[4-(4-methylpiperazin-1-yl)phenyl]aminol}-8-[(1-methylpyrazol-4-yl)methyl]pyrido[2,3-d]pyrimidin-7-one

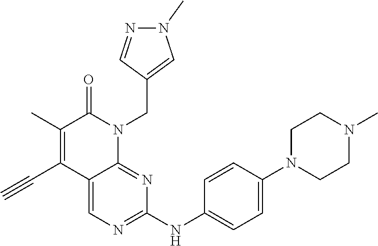 [SMILES: C#Cc1c(C)c(=O)n(Cc2cnn(C)c2)c2nc(Nc3ccc(N4CCN(C)CC4)cc3)ncc12]

General procedure 15 was applied to 6-methyl-2-{[4-(4-methylpiperazin-1-yl)phenyl]amino}-8-[(1-methylpyrazol-4-yl)methyl]-5-[2-(triisopropylsilyl)ethynyl]pyrido[2,3-d]pyrimidin-7-one (90.0 mg, 0.144 mmol) and potassium fluoride (83.7 mg, 1.44 mmol) in THF (2.0 mL), DMF (2.0 mL) and water (0.50 mL), stirred for 1 hour at 60° C. The residue was purified by reverse phase flash column chromatography eluting with acetonitrile (10-50%) in water (0.1% formic acid), to yield the title compound as a yellow solid (36.6 mg, 54%).

(ES, m/z): [M+H]⁺=469.15

¹H-NMR (400 MHz, DMSO-d₆) δ 9.91 (s, 1H), 8.73 (s, 1H), 7.62 (d, J=8.9 Hz, 2H), 7.49 (s, 1H), 7.32 (s, 1H), 7.00-6.90 (m, 2H), 5.26 (s, 2H), 5.20 (s, 1H), 3.69 (s, 4H), 3.10 (dd, J=6.2, 3.7 Hz, 4H), 2.47 (d, J=5.0 Hz, 3H), 2.23 (d, J=3.6 Hz, 6H).

### Example 181

### tert-Butyl 3-[(methanesulfonyloxy)methyl]azetidine-1-carboxylate

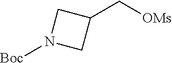 [SMILES: CC(C)(C)OC(=O)N1CC(COS(C)(=O)=O)C1]

A solution of tert-butyl 3-(hydroxymethyl)azetidine-1-carboxylate (20.0 g, 107 mmol, 1.0 eq.), methanesulfonyl chloride (13.5 g, 117 mmol, 1.1 eq.) and triethylamine (21.6 g, 213 mmol, 2.0 eq.) in dichloromethane (200 mL) was stirred overnight at room temperature. The resulting mixture was extracted with dichloromethane (3×200 mL). The combined organic layers were washed with brine (2×100 mL), dried (Na₂SO₄) and concentrated under reduced pressure to yield the title compound as a colourless oil (27.0 g, 95%).

### tert-Butyl 3-[(methylsulfanyl)methyl]azetidine-1-carboxylate

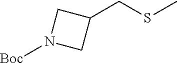

A solution of tert-butyl 3-[(methanesulfonyloxy)methyl]azetidine-1-carboxylate (27.0 g, 102 mmol, 1.0 eq.) and sodium methanethiolate (14.2 g, 203 mmol, 2.0 eq.) in DMF (270 mL) was stirred overnight at room temperature. The resulting mixture was extracted with ethyl acetate (2×200 mL). The combined organic layers were washed with brine (3×200 mL), dried (Na₂SO₄) and concentrated under reduced pressure. The residue was purified by reverse phase flash column chromatography eluting with acetonitrile (10-50%) in water (0.1% formic acid), to yield the title compound as a colourless oil (12.0 g, 54%).

### tert-Butyl 3-(methanesulfonylmethyl)azetidine-1-carboxylate

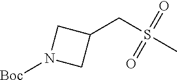 [SMILES: CC(C)(C)OC(=O)N1CC(CS(C)(=O)=O)C1]

To a stirred solution of tert-butyl 3-[(methylsulfanyl)methyl]azetidine-1-carboxylate (6.00 g, 27.6 mmol, 1.0 eq.) in dichloromethane (60 mL) was added mCPBA (10.5 g, 60.7 mmol, 2.2 eq.) portionwise at 0° C. The resulting mixture was stirred for 2 hours at room temperature then diluted with dichloromethane (300 mL) and washed with saturated aqueous sodium hydrogen carbonate solution (2×200 mL). The combined organic layers were washed with brine (2×100 mL), dried (Na₂SO₄) and concentrated under reduced pressure to yield the title compound as a white solid (6.00 g, 87%).

### 3-(Methansulfonylmethyl)azetidine hydrochloride

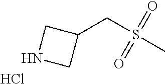

A solution of tert-butyl 3-(methanesulfonylmethyl)azetidine-1-carboxylate (6.00 g, 24.1 mmol, 1.0 eq.) in hydrochloric acid in methanol (50 mL) was stirred for 2 hours at room temperature. The resulting mixture was concentrated under reduced pressure to yield the title compound, which used in the next step without further purification.

### 3-(Methanesulfonylmethyl)-1-[(3-nitrophenyl)methyl]azetidine

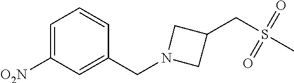 [SMILES: CS(=O)(=O)CC1CN(Cc2cccc([N+](=O)[O-])c2)C1]

A solution of 3-(methanesulfonylmethyl)azetidine (2.00 g, 13.4 mmol, 1.0 eq.), 1-(bromomethyl)-3-nitrobenzene (3.47 g, 16.1 mmol, 1.2 eq.) and cesium carbonate (13.1 g, 40.2 mmol, 3.0 eq.) in acetonitrile (200 mL) was stirred for 2 hours at 80° C. The residue was purified by flash column chromatography eluting with ethyl acetate (30%) in 40-60 petroleum ether to afford the title compound as a yellow solid (700 mg, 18%).

### 3-([3-(Methanesulfonylmethyl)azetidin-1-yl]methylaniline

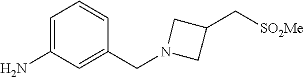 [SMILES: CS(=O)(=O)CC1CN(Cc2cccc(N)c2)C1]

General procedure 12 was applied to 3-(methanesulfonylmethyl)-1-[(3-nitrophenyl)methyl]azetidine (700 mg, 2.46 mmol) and palladium on carbon (140 mg, 1.32 mmol) in THF (7 mL) to yield the title compound as a brown yellow solid (570 mg, 91%).

### 8-Cyclopentyl-2-[(3-([3-(methanesulfonylmethyl)azetidin-1-yl]methylphenyl)amino]-5-[2-(triisopropylsilyl)ethynyl]pyrido[2,3-d]pyrimidin-7-one

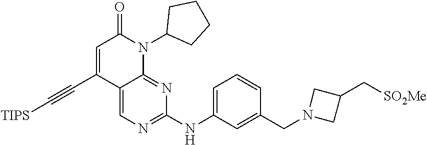

General procedure 13 was applied to 8-cyclopentyl-2-methanesulfonyl-5-[2-(triisopropylsilyl)ethynyl]pyrido[2,3-d]pyrimidin-7-one (200 mg, 0.422 mmol), 3-([3-(methanesulfonylmethyl)azetidin-1-yl]methylaniline (161 mg, 0.633 mmol) and trifluoroacetic acid (62.7 uL, 0.844 mmol) and 2-butanol (2 mL), stirred overnight at 50° C. The residue was purified by reverse phase flash column chromatography eluting with acetonitrile (10-65%) in water (0.1% formic acid), to afford the title compound as a brown solid (76.3 mg, 28%).

### 8-Cyclopentyl-5-ethynyl-2-[(3-([3-(methanesulfonylmethyl)azetidin-1-yl]methylphenyl)amino]pyrido[2,3-d]pyrimidin-7-one

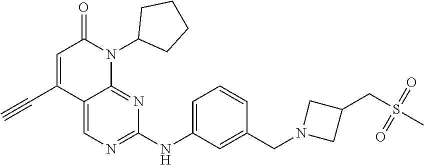 [SMILES: C#Cc1cc(=O)n(C2CCCC2)c2nc(Nc3cccc(CN4CC(CS(C)(=O)=O)C4)c3)ncc12]

General procedure 15 was applied to 8-cyclopentyl-2-[(3-([3-(methanesulfonylmethyl)azetidin-1-yl]methylphenyl)amino]-5-[2-(triisopropylsilyl)ethynyl]pyrido[2,3-d]pyrimidin-7-one (10.0 mg, 15.0 μmol) and potassium fluoride (8.97 mg, 0.150 mmol) in THF (1.0 mL), DMF (1.0 mL) and water (10 uL), stirred for 2 hours at 60° C. The residue was purified by reverse phase flash column chromatography eluting with acetonitrile (10-55%) in water (0.1% ammonium acetate), to yield the title compound as a yellow solid (13.4 mg, 25.23%).

(ES, m/z): [M+H]⁺=492.25

¹H NMR (400 MHz, DMSO-d₆) δ 10.09 (s, 1H), 9.17 (s, 1H), 7.83 (d, J=8.2 Hz, 1H), 7.71 (s, 1H), 7.27 (t, J=7.9 Hz, 1H), 6.91 (s, 1H), 6.90 (s, 1H), 5.60 (d, J=6.7 Hz, 1H), 5.04 (s, 1H), 3.54 (s, 2H), 3.47 (t, J=7.0 Hz, 2H), 3.40 (d, J=7.3 Hz, 2H), 2.96 (t, J=6.9 Hz, 2H), 2.92 (s, 3H), 2.85 (p, J=7.1 Hz, 1H), 2.03 (dt, J=13.6, 6.5 Hz, 2H), 1.78 (s, 2H), 1.75 (s, 2H), 1.63 (s, 2H).

### Example 182

### tert-Butyl 3-([(4-methylbenzenesulfonyl)oxy]methylpyrrolidine-1-carboxylate

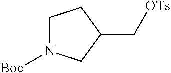 [SMILES: Cc1ccc(S(=O)(=O)OCC2CCN(C(=O)OC(C)(C)C)C2)cc1]

A solution of tert-butyl 3-(hydroxymethyl)pyrrolidine-1-carboxylate (10.0 g, 49.7 mmol, 1.0 eq.) and 4-toluenesulfonyl chloride (11.4 g, 59.6 mmol, 1.2 eq.) in dichloromethane (100 mL) was stirred for 16 hours at room temperature. The resulting mixture was washed with saturated aqueous sodium hydrogen carbonate solution (2×200 mL). The combined organic layers were washed with brine (2×200 mL), dried (Na₂SO₄) and concentrated under reduced pressure to yield the title compound as a colourless oil (10.0 g, 56%).

### tert-Butyl 3-(sulfanylmethyl)pyrrolidine-1-carboxylate

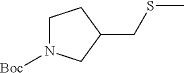

A solution of tert-butyl 3-[(methanesulfonyloxy)methyl]pyrrolidine-1-carboxylate (2.00 g, 7.16 mmol, 1.0 eq.) and (methylsulfanyl)sodium (1.00 g, 14.3 mmol, 2.0 eq.) in methanol (20 mL) was stirred for 16 hours at room temperature. The resulting mixture was extracted with ethyl acetate (2×50 mL). The combined organic layers were washed with brine (2×50 mL), dried (Na₂SO₄) and concentrated under reduced pressure. The residue was purified by flash column chromatography eluting with ethyl acetate (10%) in 40-60 petroleum ether afford the title compound as a colorless oil (1.20 g, 77%).

### tert-Butyl 3-(methanesulfonylmethyl)pyrrolidine-1-carboxylate

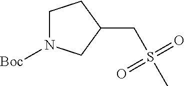 [SMILES: CC(C)(C)OC(=O)N1CCC(CS(C)(=O)=O)C1]

A mixture of tert-butyl 3-[(methylsulfanyl)methyl]pyrrolidine-1-carboxylate (6.00 g, 25.9 mmol, 1.0 eq.) and oxone (17.4 g, 103 mmol, 4.0 eq.) in THF (60 mL) and water (60 mL) was stirred for 3 hours at room temperature. The resulting mixture was extracted with ethyl acetate (3×500 mL). The combined organic layers were washed with brine (2×200 mL), dried (Na₂SO₄) and concentrated under reduced pressure to yield the title compound as a colourless oil (6.00 g, 88%).

### 3-(Methanesulfonylmethyl)pyrrolidine

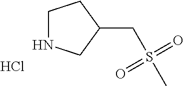

A solution of tert-butyl 3-(methanesulfonylmethyl)pyrrolidine-1-carboxylate (6.00 g, 22.8 mmol, 1.0 eq.) in hydrochloric acid in methanol (60 mL) was stirred for 2 hours at room temperature. The resulting mixture was concentrated under reduced pressure to yield the title compound as a colourless oil (4.00 g), used in the next step without further purification.

### 3-(Methanesulfonylmethyl)-1-[(3-nitrophenyl)methyl]pyrrolidine

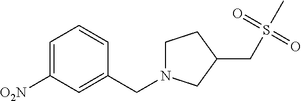 [SMILES: CS(=O)(=O)CC1CCN(Cc2cccc([N+](=O)[O-])c2)C1]

A solution of 3-(methanesulfonylmethyl)pyrrolidine (600 mg, 3.67 mmol, 1.0 eq.), cesium carbonate (2.39 g, 7.35 mmol, 2.0 eq.) and 3-nitrobenzaldehyde (666 mg, 4.41 mmol, 1.2 eq.) in acetonitrile (6.0 ml) was stirred for 2 hours at 80° C. The residue was purified by flash column chromatography eluting with ethyl acetate (50%) in 40-60 petroleum ether to afford the title compound as a yellow solid (600 mg, 55%).

### 3-([3-(Methanesulfonylmethyl)pyrrolidin-1-yl]methylaniline

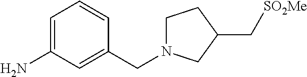 [SMILES: CS(=O)(=O)CC1CCN(Cc2cccc(N)c2)C1]

General procedure 12 was applied to 3-(methanesulfonylmethyl)-1-[(3-nitrophenyl)methyl]pyrrolidine (600 mg, 2.01 mmol) and palladium on carbon (120 mg, 1.13 mmol) in methanol (30 mL) to yield the title compound as a white solid (500 mg, 92%).

### 8-Cyclopentyl-2-[(3-([3-(methanesulfonylmethyl)pyrrolidin-1-yl]methylphenyl)amino]-5-[2-(triisopropylsilyl)ethynyl]pyrido[2,3-d]pyrimidin-7-one

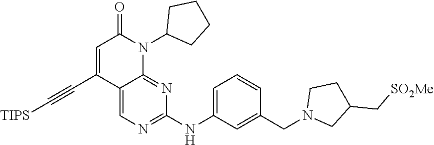

General procedure 13 was applied to 8-cyclopentyl-2-methanesulfonyl-5-[2-(triisopropylsilyl)ethynyl]pyrido[2,3-d]pyrimidin-7-one (350 mg, 0.739 mmol), 3-([3-(methanesulfonylmethyl)pyrrolidin-1-yl]methylaniline (297 mg, 1.11 mmol) and trifluoroacetic acid (168 mg, 1.48 mmol) in butan-2-ol (7.0 mL), stirred overnight at room temperature. The residue was purified by reverse phase flash column chromatography eluting with acetonitrile (22-43%) in water (0.1% formic acid), to afford the title compound as a yellow solid (300 mg, 61%).

### 8-Cyclopentyl-5-ethynyl-2-[(3-([3-(methanesulfonylmethyl)pyrrolidin-1-yl]methylphenyl)amino]pyrido[2,3-d]pyrimidin-7-one

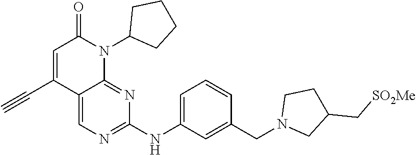 [SMILES: C#Cc1cc(=O)n(C2CCCC2)c2nc(Nc3cccc(CN4CCC(CS(C)(=O)=O)C4)c3)ncc12]

General procedure 15 was applied to 8-cyclopentyl-2-[(3-([3-(methanesulfonylmethyl)pyrrolidin-1-yl]methylphenyl)amino]-5-[2-(triisopropylsilyl)ethynyl]pyrido[2,3-d]pyrimidin-7-one (200 mg, 0.302 mmol) and potassium fluoride (175 mg, 3.02 mmol) in water (20 uL) and DMF (2 mL), stirred for 1 hour at room temperature. The crude product was purified by Prep-HPLC eluting with acetonitrile (22-43%) in water (0.1% formic acid), to afford the title compound as a yellow solid (45.0 mg, 29%).

(ES, m/z): [M+H]⁺=506.15;

¹H-NMR (400 MHz, DMSO-d₆) δ 10.17 (s, 1H), 9.18 (s, 1H), 8.14 (d, J=1.1 Hz, 1H), 7.96-7.84 (m, 1H), 7.78 (s, 1H), 7.33 (t, J=7.8 Hz, 1H), 7.01 (d, J=7.5 Hz, 1H), 6.91 (s, 1H), 5.60 (dq, J=6.3, 3.3 Hz, 1H), 5.06 (s, 1H), 3.78 (s, 2H), 3.27 (dd, J=7.0, 4.7 Hz, 2H), 2.97 (s, 4H), 2.81-2.62 (m, 3H), 2.12 (dt, J=14.4, 7.4 Hz, 1H), 2.07-1.98 (m, 2H), 1.83-1.70 (m, 4H), 1.69-1.58 (m, 3H).

### Example 183

### tert-Butyl 4-([(4-methylbenzenesulfonyl)oxy]methylpiperidine-1-carboxylate

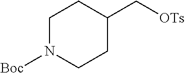 [SMILES: Cc1ccc(S(=O)(=O)OCC2CCN(C(=O)OC(C)(C)C)CC2)cc1]

To a stirred solution of tert-butyl 4-(hydroxymethyl)piperidine-1-carboxylate (20.0 g, 92.9 mmol, 1.0 eq.) in dichloromethane (200 mL) was added triethylamine (32.0 mL, 230 mmol, 2.5 eq.) and toluenesulfonyl chloride (35.4 g, 186 mmol, 2.0 eq.) dropwise at room temperature. The reaction mixture was irradiated with microwave radiation overnight at room temperature. The residue was purified by flash column chromatography eluting with ethyl acetate (12%) in 40-60 petroleum ether to afford the title compound as a yellow oil (27.0 g, 79%).

### tert-Butyl 4-[(methylsulfanyl)methyl]piperidine-1-carboxylate

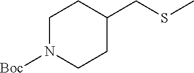 [SMILES: CSCC1CCN(C(=O)OC(C)(C)C)CC1]

To a stirred solution of tert-butyl 4-[(methanesulfonyloxy)methyl]piperidine-1-carboxylate (27.0 g, 92.0 mmol, 1.0 eq.) in DMF (71 ml) was added sodium methanethiolate (19.4 g, 276 mmol, 3.0 eq.) at room temperature. The resulting mixture was stirred overnight at room temperature then extracted with ethyl acetate (3×200 mL). The combined organic layers were washed with brine (2×250 mL), dried (Na₂SO₄) and concentrated under reduced pressure. The residue was purified by flash column chromatography eluting with ethyl acetate (20-25%) in 40-60 petroleum ether to afford the title compound as a yellow oil (16.4 g, 72%).

### tert-Butyl 4-(methanesulfonylmethyl)piperidine-1-carboxylate

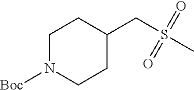 [SMILES: CC(C)(C)OC(=O)N1CCC(CS(C)(=O)=O)CC1]

To a stirred solution of tert-butyl 4-[(methylsulfanyl)methyl]piperidine-1-carboxylate (10.5 g, 42.8 mmol, 1.0 eq.) in dichloromethane (105 mL) added mCPBA (18.5 g, 107 mmol, 2.5 eq.) at 0-5° C. The resulting mixture was stirred for 30 minutes at room temperature then washed with saturated aqueous sodium hydrogen carbonate solution (400 mL). The aqueous layer was extracted with dichloromethane (3×150 mL), the combined organic layers were washed with brine (400 mL), dried (Na₂SO₄) and concentrated under reduced pressure. The residue was purified by flash column chromatography eluting with ethyl acetate (25-35%) in 40-60 petroleum ether to afford as a white solid (8.78 g, 78%).

### 4-(Methanesulfonylmethyl)piperidine

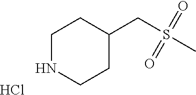

A solution of tert-butyl 4-(methanesulfonylmethyl)piperidine-1-carboxylate (8.80 g, 31.7 mmol, 1.0 eq.) and hydrochloric acid in methanol (88 mL, 4M) was stirred for 4 hours at room temperature. The resulting mixture was concentrated under reduced pressure, to afford the title compound as a white solid (8.39 g, 149%) used in the next step without further purification.

### 4-(Methanesulfonylmethyl)-1-[(3-nitrophenyl)methyl]piperidine

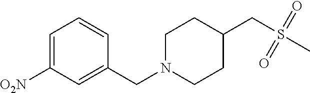 [SMILES: CS(=O)(=O)CC1CCN(Cc2cccc([N+](=O)[O-])c2)CC1]

A solution of 4-(methanesulfonylmethyl)piperidine (2.00 g, 11.3 mmol, 1.0 eq.), 3-(bromomethyl)benzaldehyde (2.47 g, 12.4 mmol, 1.1 eq.) and cesium carbonate (11.0 g, 33.8 mmol, 3.0 eq.) in acetonitrile (20 mL) was stirred for 2 hours at 50° C. then concentrated under reduced pressure. The residue was purified by flash column chromatography eluting with ethyl acetate (30%) in 40-60 petroleum ether to afford the title compound as a brown oil (1.89 g, 54%).

### 3-([4-(Methanesulfonylmethyl)piperidin-1-yl]methylaniline

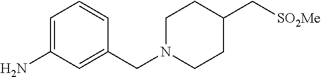 [SMILES: CS(=O)(=O)CC1CCN(Cc2cccc(N)c2)CC1]

General procedure 12 was applied to 4-(methanesulfonylmethyl)-1-[(3-nitrophenyl)methyl]piperidine (1.80 g, 5.76 mmol) and palladium on carbon (1.22 g, 1.15 mmol) in methanol (30 mL) to afford the title compound as a brown oil (1.49 g, 92%).

### 8-Cyclopentyl-2-[(3-([4-(methanesulfonylmethyl)piperidin-1-yl]methylphenyl)amino]-5-[2-(triisopropylsilyl)ethynyl]pyrido[2,3-d]pyrimidin-7-one

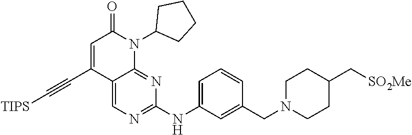

General procedure 13 was applied to 8-cyclopentyl-2-methanesulfonyl-5-[2-(triisopropylsilyl)ethynyl]pyrido[2,3-d]pyrimidin-7-one (300 mg, 0.633 mmol), 3-([4-(methanesulfonylmethyl)piperidin-1-yl]methylaniline (268 mg, 0.950 mmol) and trifluoroacetic acid (94.1 μL, 1.27 mmol) and 2-butanol (3 mL), stirred overnight at 100° C. The residue was purified by reverse phase flash column chromatography eluting with acetonitrile (10-80%) in water (0.1% trifluoroacetic acid), to yield the title compound as a brown solid (256 mg, 60%).

### 8-Cyclopentyl-5-ethynyl-2-[(3-([4-(methanesulfonylmethyl)piperidin-1-yl]methylphenyl)amino]pyrido[2,3-d]pyrimidin-7-one

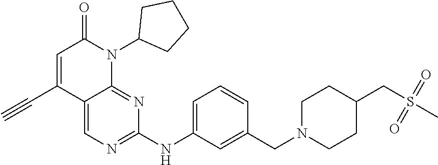 [SMILES: C#Cc1cc(=O)n(C2CCCC2)c2nc(Nc3cccc(CN4CCC(CS(C)(=O)=O)CC4)c3)ncc12]

General procedure 15 was applied to 8-cyclopentyl-2-[(3-([4-(methanesulfonylmethyl)piperidin-1-yl]methylphenyl)amino]-5-[2-(triisopropylsilyl)ethynyl]pyrido[2,3-d]pyrimidin-7-one (150 mg, 0.222 mmol) and potassium fluoride (129 mg, 2.22 mmol) in THF (1.5 mL), DMF (1.5 mL) and water (150 uL), stirred for 1 hour at 60° C. The residue was purified by reverse phase flash column chromatography eluting with acetonitrile (10-50%) in water (0.1% formic acid), to yield the title compound as a yellow solid (41.9 mg, 36%).

(ES, m/z): [M+H]⁺=520.25

¹H NMR (400 MHz, DMSO-d₆) b 10.09 (s, 1H), 9.17 (s, 1H), 7.86 (d, J=8.2 Hz, 1H), 7.73 (s, 1H), 7.29 (t, J=7.8 Hz, 1H), 6.95 (d, J=7.5 Hz, 1H), 6.90 (s, 1H), 5.59 (d, J=6.8 Hz, 1H), 5.05 (s, 1H), 3.45 (s, 2H), 3.08 (d, J=6.3 Hz, 2H), 2.96 (s, 3H), 2.82 (d, J=11.3 Hz, 2H), 2.00 (t, J=11.3 Hz, 4H), 1.91 (s, 1H), 1.86-1.69 (m, 6H), 1.63 (s, 2H), 1.34 (q, J=10.9 Hz, 2H).

### Example 184

### 8-Cyclopentyl-2-[(2-methvl-1,3-dihydroisoindol-4-yl)amino]-5-[2-(triisopropylsilyl)ethynyl]pyrido[2,3-d]pyrimidin-7-one

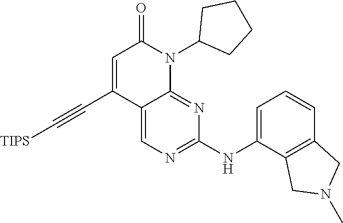

General procedure 13 was applied to 8-cyclopentyl-2-methanesulfonyl-5-[2-(triisopropylsilyl)ethynyl]pyrido[2,3-d]pyrimidin-7-one (500 mg, 1.06 mmol), 2-methyl-1,3-dihydroisoindol-4-amine (203 mg, 1.37 mmol) and trifluoroacetic acid (0.120 mL, 1.58 mmol) in dioxane (10 mL), stirred overnight at 100° C. The residue was purified by reverse phase flash column chromatography eluting with acetonitrile (10-50%) in water (0.1% formic acid), to yield the title compound as a black solid (110 mg, 19%).

### 8-Cyclopentyl-5-ethynyl-2-[(2-methvl-1,3-dihydroisoindol-4-yl)amino]pyrido[2,3-d]pyrimidin-7-one

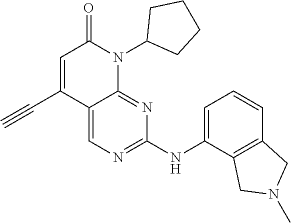 [SMILES: C#Cc1cc(=O)n(C2CCCC2)c2nc(Nc3cccc4c3CN(C)C4)ncc12]

General procedure 15 was applied to 8-cyclopentyl-2-[(2-methyl-1,3-dihydroisoindol-4-yl)amino]-5-[2-(triisopropylsilyl)ethynyl]pyrido[2,3-d]pyrimidin-7-one (100 mg, 0.185 mmol) and potassium fluoride (53.6 mg, 0.925 mmol) in DMF (2.0 mL), stirred for 30 minutes at 60° C. The residue was purified by reverse phase flash column chromatography eluting acetonitrile (10-70%) in water (0.1% formic acid), to yield the title compound as an orange solid (7.60 mg, 11%).

(ES, m/z) [M+H]⁺=386.10

¹H-NMR (300 MHz, DMSO-d₆) δ 9.77 (s, 1H), 9.15 (s, 1H), 7.48 (d, J=7.8 Hz, 1H), 7.26 (t, J=7.7 Hz, 1H), 7.08 (d, J=7.3 Hz, 1H), 6.89 (s, 1H), 5.59-5.51 (m, 1H), 5.05 (s, 1H), 4.03 (d, J=13.7 Hz, 4H), 2.58 (s, 3H), 2.06-1.90 (m, 2H), 1.81-1.68 (m, 4H), 1.65 (d, J=15.7 Hz, 2H).

### Example 185

### 5-Nitro-1,2,3,4-tetrahydroisoquinoline

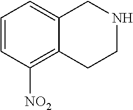 [SMILES: O=[N+]([O-])c1cccc2c1CCNC2]

A solution of 5-nitroisoquinoline (10.0 g, 57.4 mmol, 1.0 eq.) and sodium borohydride (21.7 g, 574 mmol, 10 eq.) in acetic acid (98 mL) was stirred for 5 minutes at 0° C. then for 15 minutes at room temperature. The mixture was basified to pH 9 with sodium hydroxide. The aqueous layer was extracted with ethyl acetate (100 mL) then washed with water (5×100 mL) and concentrated under reduced pressure to yield the title compound as a black green solid (6.50 g, 63%).

### 2-Methyl-5-nitro-3,4-dihydro-1H-isoquinoline

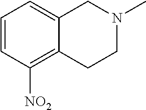

A solution of 5-nitro-1,2,3,4-tetrahydroisoquinoline (1.30 g, 7.29 mmol, 1.0 eq.), formaldehyde solution (2.19 g, 72.9 mmol, 10 eq.), acetic acid (40.0 mg, 0.730 mmol, 0.10 eq.) and sodium borohydride (1.38 g, 36.5 mmol, 5 eq.) in methanol (20 mL) was stirred for 30 minutes at room temperature. The resulting mixture was diluted with water (100 mL) and extracted with ethyl acetate (100 mL). The organic layer was concentrated under reduced pressure to yield the title compound as a black solid (865 mg, 61%).

### 2-Methvl-3,4-dihydro-1H-isoquinolin-5-amine

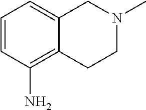

General procedure 12 was applied to 2-methyl-5-nitro-3,4-dihydro-1H-isoquinoline (860 mg, 4.47 mmol) and palladium on carbon (230 mg, 0.216 mmol) in dichloromethane (20 mL) to yield the title compound as a green solid (624 mg, 86%).

### 8-Cyclopentyl-2-[(2-methyl-3,4-dihydro-1H-isoquinolin-5-yl)amino]-5-[2-(triisopropylsilyl)ethynyl]pyrido[2,3-d]pyrimidin-7-one

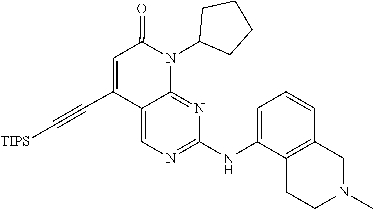

General procedure 13 was applied to 8-cyclopentyl-2-methanesulfonyl-5-[2-(triisopropylsilyl)ethynyl]pyrido[2,3-d]pyrimidin-7-one (350 mg, 0.739 mmol), 2-methyl-3,4-dihydro-1H-isoquinolin-5-amine (144 mg, 0.887 mmol) and trifluoroacetic acid (0.110 mL, 1.48 mmol) in 2-methyl-2-butanol (5.0 mL) was stirred for 1 hour at 100° C. The residue was purified by reverse phase flash column chromatography eluting with acetonitrile (10-50%) in water (0.1% formic acid), to yield the title compound as a yellow solid (112 mg, 27%).

### 8-Cyclopentyl-5-ethynyl-2-[(2-methyl-3,4-dihydro-1H-isoquinolin-5-yl)amino]pyrido[2,3-d]pyrimidin-7-one

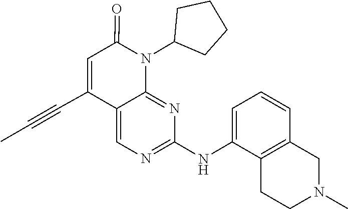 [SMILES: CC#Cc1cc(=O)n(C2CCCC2)c2nc(Nc3cccc4c3CCN(C)C4)ncc12]

General procedure 15 applied to 8-cyclopentyl-2-[(2-methyl-3,4-dihydro-1H-isoquinolin-5-yl)amino]-5-[2-(triisopropylsilyl)ethynyl]pyrido[2,3-d]pyrimidin-7-one (98.0 mg, 0.176 mmol) and potassium fluoride (51.2 mg, 0.880 mmol) in THF (1.0 mL), DMF (1.0 mL), stirred for 30 minutes at 60° C. The residue was purified by reverse phase flash column chromatography eluting with acetonitrile (10-60%) in water (0.1% formic acid), to yield the title compound as a yellow solid (4.70 mg, 6%).

(ES, m/z) [M+H]⁺=400.05

¹H-NMR (300 MHz, DMSO-d₆) δ 9.30 (s, 1H), 9.09 (s, 1H), 7.34 (d, J=7.7 Hz, 1H), 7.17 (t, J=7.7 Hz, 1H), 6.95 (d, J=7.6 Hz, 1H), 6.83 (s, 1H), 5.54 (s, 1H), 5.02 (s, 1H), 3.51 (s, 2H), 2.74 (d, J=5.7 Hz, 2H), 2.56 (d, J=6.0 Hz, 2H), 2.33 (s, 3H), 1.95 (s, 2H), 1.77-1.64 (m, 4H), 1.60 (d, J=2.8 Hz, 2H).

### Example 186

### 8-(3-(^{t}Butyldiphenylsilyl)oxy)propyl)-2-(methylthio)-5-((trisopropylsilyl)ethynyl)pyrido[2,3-d]pyrimidin-7(8H)-one

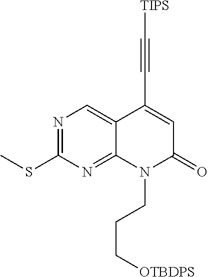

General procedure 18 was applied to 2-(methylthio)-5-((triisopropylsilyl)ethynyl)pyrido[2,3-d]pyrimidin-7(8H)-one (500 mg, 1.34 mmol) with tButyldiphenylsilyl(oxy)propanol (506 mg, 1.61 mmol), triphenylphosphine (527 mg, 2.01 mmol) and DIAD (0.400 mL, 2.01 mmol) in dichloromethane (14 mL). The crude material was purified by flash column chromatography eluting with ethyl acetate (0-30%) in 40-60 petroleum ether to yield the title compound as a colourless oil (834 mg, 1.24 mmol, 77%).

(ES, m/z): [M+H]⁺=670.5

### 8-(3-(^{t}Butyldiphenylsilyl)oxy)propyl)-2-(methylsulfonyl)-5-((trisopropylsilyl)ethynyl)pyrido[2,3-d]pyrimdin-7(8H)-one

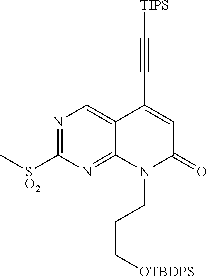

General procedure 10 was applied to 8-(3-(t-butyldiphenylsilyl)oxy)propyl)-2-(methylthio)-5-((trisopropylsilyl)ethynyl)pyrido[2,3-d]pyrimdin-7(8H)-one (833 mg, 1.24 mmol) with m-CPBA (644 mg, 3.73 mmol) in dichloromethane (12 mL). The crude title compound was carried forward without further purification (767 mg, 1.64 mmol, 100%).

### 2-((3-hydroxyphenyl)amino)-8-(3-hydroxypropyl)-5-((triisopropyisiyl)ethynyl)pyrido[2,3-d]pyrimidin-7(8H)-one

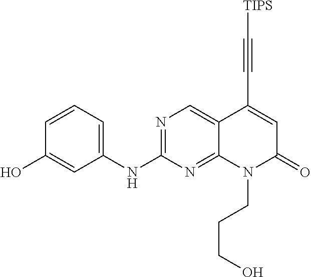

General Procedure 13 was applied to 8-(3-(t-butyldiphenylsilyl)oxy)propyl)-2-(methylsulfonyl)-5-((trisopropylsilyl)ethynyl)pyrido[2,3-d]pyrimdin-7(8H)-one (767 mg, 1.09 mmol) with 3-aminophenol (179 mg, 1.64 mmol) and trifluoroacetic acid (130 μL, 1.64 mmol) in acetonitrile (5.0 mL). The crude material was purified by flash column chromatography eluting with methanol (0-20%) in dichloromethane to yield the title compound as an orange solid (119 mg, 0.242 mmol, 22%).

(ES, m/z): [M+H]⁺=493.4

### 1⁵-((triisopropylsilyl)ethynyl)-1⁷,1⁸-dihydro-4-oxa-2-aza-1(2,8)-pyrido[2,3-d]pyrimidina-3(1,3)-benzenacycloheptaphan-1⁷-one

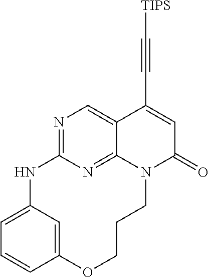

General procedure 20 was applied to 2-((3-hydroxyphenyl)amino)-8-(3-hydroxypropyl)-5-((triisopropylsilyl)ethynyl)pyrido[2,3-d]pyrimidin-7(8H)-one (361 mg, 0.733 mmol) with triphenylphosphine (769 mg, 2.93 mmol) and DIAD (0.580 mL, 2.93 mmol) in THF (14 mL). The crude material was purified by flash column chromatography eluting with methanol (0-10%) in dichloromethane to yield the title compound as an orange solid (108 mg, 0.228 mmol, 31%).

(ES, m/z): [M+H]⁺=475.4

### 1⁵-ethynyl-1⁷,1⁸-dihydro-4-oxa-2-aza-1(2,8)-pyrido[2,3-d]pyrimidina-3(1,3)-benzenacycloheptaphan-1⁷-one

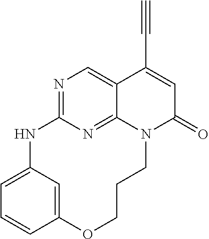 [SMILES: C#Cc1cc(=O)n2c3nc(ncc13)Nc1cccc(c1)OCCC2]

General procedure 15 was applied to 1⁵-((triisopropylsilyl)ethynyl)-1⁷,1⁸-dihydro-4-oxa-2-aza-1(2,8)-pyrido[2,3-d]pyrimidina-3(1,3)-benzenacycloheptaphan-1⁷-one (108 mg, 0.228 mmol) with potassium fluoride (265 mg, 4.56 mmol) in DMF (2.3 mL). The crude material was purified by flash column chromatography eluting with methanol (0-20%) in dichloromethane to yield the title compound as an orange solid (15.2 mg, 47.8 μmol, 21%).

(ES, m/z): [M+H]⁺=319.2

¹H NMR (500 MHz, DMSO-d₆) δ 9.09 (s, 1H), 8.45 (s, 1H), 7.04-6.92 (m, 1H), 6.40-6.30 (m, 3H), 6.27 (s, 1H), 4.99 (s, 1H), 3.98-3.86 (m, 4H), 2.24-2.12 (m, 2H).

### Example 187

### (Z)-4-((tert-butyldiphenylsilyl)oxy)but-2-en-1-ol

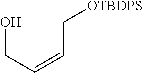

General procedure 21 was applied to Z-2-butene-1,4-diol (5.50 g, 62.5 mmol) with sodium hydride (2.50 g, 62.5 mmol) and tert-butyldiphenylsilylchloride (16.3 mL, 62.5 mmol) in THF (125 mL). The crude material was purified by flash column chromatography eluting with ethyl acetate (0-10%) in 40-60 petroleum ether to yield the title compound as a colourless oil (14.3 g, 43.8 mmol, 70%).

### (Z)-8-(4-((tert-butyldiphenylsilyl)oxy)but-2-en-1-yl)-2-(methylthio)-5-((triisopropylsilyl)ethynyl)pyrido[2,3-d]pyrimidin-7(8H)-one

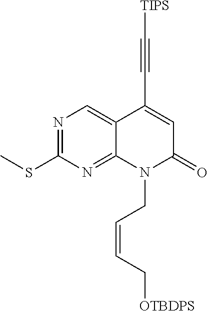

General procedure 18 was applied to 2-(methylthio)-5-((triisopropylsilyl)ethynyl)pyrido[2,3-d]pyrimidin-7(8H)-one (500 mg, 1.34 mmol) with (Z)-4-((tert-butyldiphenylsilyl)oxy)but-2-en-1-ol (202 mg, 1.61 mmol), triphenylphosphine (633 mg, 2.40 mmol) and DIAD (452 μL, 2.40 mmol) in dichloromethane (13 mL). The crude material was purified by flash column chromatography eluting with ethyl acetate (0-30%) in 40-60 petroleum ether to yield the title compound as a colourless oil (602 mg, 1.07 mmol, 81%).

(ES, m/z): [M+H]⁺=558.5

### (Z)-8-(4-((tert-butyldiphenylsilyl)oxy)but-2-en-1-yl)-2-(methylsulfonyl)-5-((triisopropylsilyl)ethynyl)pyrido[2,3-d]pyrimidin-7(8H)-one

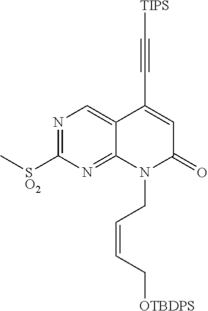

General procedure 10 was applied to (Z)-8-(4-((tert-butyldiphenylsilyl)oxy)but-2-en-1-yl)-2-(methylthio)-5-((triisopropylsilyl)ethynyl)pyrido[2,3-d]pyrimidin-7(8H)-one (346 mg, 0.621 mmol) with m-CPBA (459 mg, 1.86 mmol) in dichloromethane (6.2 mL) to yield the title compound (341 mg, 0.579 mmol, 93%) which was used without further purification.

### (Z)-8-(4-hydroxybut-2-en-1-yl)-2-((3-hydroxyphenyl)amino)-5-((triisopropylsilyl)ethynyl)pyrido[2,3-d]pyrimidin-7(8H)-one

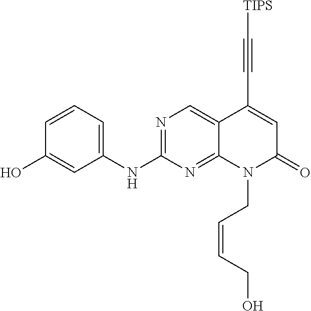

General Procedure 13 was applied to (Z)-8-(4-((tert-butyldiphenylsilyl)oxy)but-2-en-1-yl)-2-(methylsulfonyl)-5-((triisopropylsilyl)ethynyl)pyrido[2,3-d]pyrimidin-7(8H)-one (341 mg, 0.579 mmol) with 3-aminophenol (76.0 mg, 0.695 mmol) and trifluoroacetic acid (53.0 μL, 0.695 mmol) in acetonitrile (6.0 mL). The crude material was purified by flash column chromatography eluting with methanol (0-20%) in dichloromethane to yield the title compound as an orange solid (176 mg, 0.349 mmol, 50%).

(ES, m/z): [M+H]⁺=505.3
(Z)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-6-en-1⁷-one

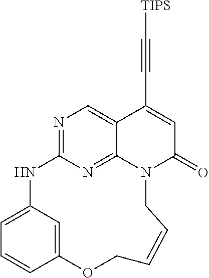

General procedure 20 was applied to (Z)-8-(4-hydroxybut-2-en-1-yl)-2-((3-hydroxyphenyl)amino)-5-((triisopropylsilyl)ethynyl)pyrido[2,3-d]pyrimidin-7(8H)-one (176 mg, 0.349 mmol) with triphenylphosphine (367 mg, 1.40 mmol) and DIAD (261 μL, 1.40 mmol) in THF (14 mL). The crude material was purified by flash column chromatography eluting with methanol (0-10%) in dichloromethane to yield the title compound as an orange solid (56.1 mg, 0.115 mmol, 33%).

(ES, m/z): [M+H]⁺=487.3
(Z)-1⁵-ethynyl-1⁷,1⁸-dihydro-4-oxa-2-aza-1(2,8)-pyrido[2,3-d]pyrimidina-3(1,3)-benzenacyclooctaphan-6-en-1⁷-one

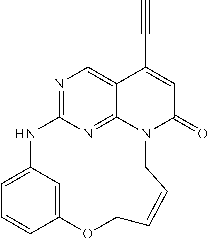 [SMILES: C#Cc1cc(=O)n2c3nc(ncc13)Nc1cccc(c1)OC/C=C\C2]

General procedure 15 was applied to (Z)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-6-en-17-one (56.1 mg, 0.115 mmol) with potassium fluoride (134 mg, 2.31 mmol) in DMF (1.2 mL).

The crude material was purified by flash column chromatography eluting with methanol (0-20%) in dichloromethane to yield the title compound as an orange solid (10.1 mg, 30.6 μmol, 27%).

(ES, m/z): [M+H]⁺=331.2

¹H NMR (500 MHz, DMSO-d₆) δ 10.51 (s, 1H), 9.42 (t, J=2.4 Hz, 1H), 8.82 (s, 1H), 7.13 (t, J=8.1 Hz, 1H), 6.78 (dd, J=7.9, 2.0 Hz, 1H), 6.67 (s, 1H), 6.51 (dd, J=7.9, 2.6 Hz, 1H), 5.68 (t, J=11.2 Hz, 1H), 5.51 (t, J=10.7 Hz, 1H), 5.18 (dd, J=14.1, 11.4 Hz, 1H), 5.11 (d, J=9.9 Hz, 2H), 5.03 (dd, J=15.8, 10.0 Hz, 1H), 4.61 (d, J=14.3 Hz, 1H).

### Example 188

### (Z)-tert-butyl((4-(2-fluoro-5-nitrophenoxy)but-2-en-1-yl)oxy)diphenylsilane

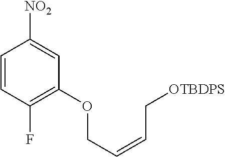

General procedure 22 was applied to 5-fluoro-3-nitrophenol (1.00 g, 6.36 mmol) with (Z)-4-((tert-butyldiphenylsilyl)oxy)but-2-en-1-ol (2.28 g, 7.00 mmol), triphenylphosphine (2.17 g, 8.27 mmol) and DIAD (1.55 mL, 8.27 mmol) in THF (5.0 mL). The crude material was purified by flash column chromatography eluting with ethyl acetate (0-10%) in 40-60 petroleum ether to yield the title compound as a white solid (1.39 g, 2.99 mmol, 47%).

### (Z)-4-(2-(4-methylpiperazin-1-yl)-5-nitrophenoxy)but-2-en-1-ol

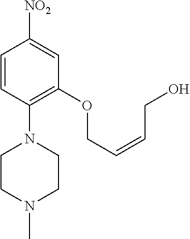 [SMILES: CN1CCN(c2ccc([N+](=O)[O-])cc2OC/C=C\CO)CC1]

General procedure 11 was applied to (Z)-tert-butyl((4-(2-fluoro-5-nitrophenoxy)but-2-en-1-yl)oxy)diphenylsilane (1.39 g, 2.99 mmol) with N-methylpiperazine (0.36 mL, 3.29 mmol) and potassium carbonate (496 mg, 3.58 mmol) in DMSO (5.0 mL). The crude material was purified by flash column chromatography eluting with ethyl acetate (10-50%) in 40-60 petroleum ether to yield the title compound as a white solid (918 mg, 2.99 mmol, 100%).

(ES, m/z): [M+H]⁺=450.2

### (Z)-1-(2-((4-((tert-butyldiphenylsilyl)oxy)but-2-en-1-yl)oxy)-4-nitrophenyl)-4-methylpiperazine

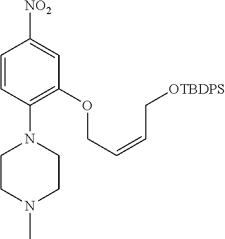

General procedure 21 was applied to (Z)-4-(2-(4-methylpiperazin-1-yl)-5-nitrophenoxy)but-2-en-1-ol (918 mg, 2.99 mmol) with sodium hydride (132 mg, 3.29 mmol) and tert-butyldiphenylsilylchloride (0.83 mL, 3.29 mmol) in THF (6.0 mL). The crude material was purified by flash column chromatography eluting with ethyl acetate (10-50%) in 40-60 petroleum ether to yield the title compound as a white solid (1.14 g, 2.09 mmol, 70%).

(ES, m/z): [M+H]⁺=547.3

### (Z)-3-((4-((tert-butyldiphenylsilyl)oxy)but-2-en-1-yl)oxy)-4-(4-methylpiperazin-1-yl)aniline

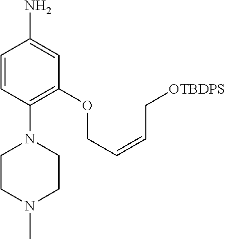

General procedure 23 was applied to (Z)-1-(2-((4-((tert-butyldiphenylsilyl)oxy)but-2-en-1-yl)oxy)-4-nitrophenyl)-4-methylpiperazine (1.14 g, 2.09 mmol) with iron (584 mg, 10.4 mmol) in ammonium chloride solution (2.6 mL) and methanol (5.2 mL). The crude material was purified by flash column chromatography eluting with methanol (0-10%) in dichloromethane to yield the title compound as a colourless oil (586 mg, 1.14 mmol, 55%).

(ES, m/z): [M+H]⁺=516.4

### 2-(methylsulfonyl)-5-((triisopropylsilyl)ethynyl)pyrido[2,3-d]pyrimidin-7(8H)-one

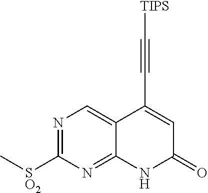

General procedure 10 was applied to 2-(methylthio)-5-((triisopropylsilyl)ethynyl)pyrido[2,3-d]pyrimidin-7(8H)-one (1.53 g, 4.11 mmol) with m-CPBA (2.03 g, 9.04 mmol) in dichloromethane (40 mL). The crude title compound was carried forward without further purification (1.54 g, 3.82 mmol, 93%).

(ES, m/z): [M+H]⁺=406.3

### (Z)-2-((3-((4-hydroxybut-2-en-1-yl)oxy)-4-(4-methylpiperazin-1-yl)phenyl)amino)-5-((triisopropylsilyl)ethynyl)pyrido[2,3-d]pyrimidin-7(8H)-one

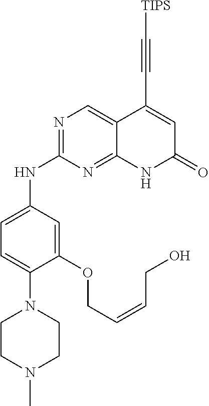

General Procedure 13 was applied to 2-(methylsulfonyl)-5-((triisopropylsilyl)ethynyl)pyrido[2,3-d]pyrimidin-7(8H)-one (384 mg, 0.947 mmol) with (Z)-3-((4-((tert-butyldiphenylsilyl)oxy)but-2-en-1-yl)oxy)-4-(4-methylpiperazin-1-yl)aniline (586 mg, 1.14 mmol) and trifluoroacetic acid (87.0 μL, 1.14 mmol) in acetonitrile (9.5 mL). The crude material was purified by flash column chromatography eluting with methanol (0-20%) in dichloromethane to yield the title compound as an orange solid (204 mg, 0.337 mmol, 36%).

(ES, m/z): [M+H]⁺=603.4

### (Z)-3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-6-en-1⁷-one

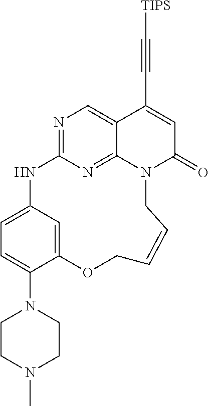

General procedure 20 was applied to (Z)-2-((3-((4-hydroxybut-2-en-1-yl)oxy)-4-(4-methylpiperazin-1-yl)phenyl)amino)-5-((triisopropylsilyl)ethynyl)pyrido[2,3-d]pyrimidin-7(8H)-one (203 mg, 0.337 mmol) with triphenylphosphine (353 mg, 1.35 mmol) and DIAD (252 μL, 1.35 mmol) in THF (14 mL). The crude material was purified by flash column chromatography eluting with methanol (0-10%) in dichloromethane to yield the title compound as an orange solid (109 mg, 0.186 mmol, 55%).

(ES, m/z): [M+H]⁺=585.5

### (Z)-1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-6-en-1⁷-one

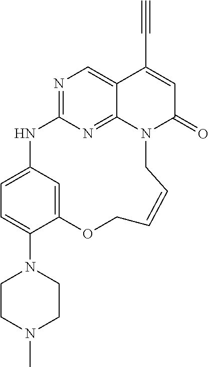 [SMILES: C#Cc1cc(=O)n2c3nc(ncc13)Nc1ccc(N3CCN(C)CC3)c(c1)OC/C=C\C2]

General procedure 15 was applied to (Z)-3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-6-en-1⁷-one (109 mg, 0.186 mmol) with potassium fluoride (21.7 mg, 0.373 mmol) in DMF (2.0 mL). The crude material was purified by flash column chromatography eluting with methanol (0-20%) in dichloromethane to yield the title compound as an orange solid (56.0 mg, 0.131 mmol, 71%).

(ES, m/z): [M+H]⁺=429.3

¹H NMR (500 MHz, DMSO-d₆) δ 10.38 (s, 1H), 9.32 (d, J=2.4 Hz, 1H), 8.77 (s, 1H), 6.79 (d, J=8.4 Hz, 1H), 6.73 (dd, J=8.5, 2.3 Hz, 1H), 6.62 (s, 1H), 5.70 (t, J=11.2 Hz, 1H), 5.49 (t, J=10.7 Hz, 1H), 5.20-5.04 (m, 3H), 5.03-4.93 (m, 1H), 4.69 (d, J=14.4 Hz, 1H), 2.96 (s, 4H), 2.26 (s, 3H).

### Example 189

### 4-((tert-butyldiphenylsilyl)oxy)butan-1-ol

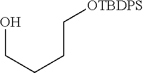

General procedure 21 was applied to butane-1,4-diol (3.00 g, 33.3 mmol) with sodium hydride (1.32 g, 33.3 mmol) and tert-butyldiphenylsilylchloride (9.15 mL, 33.3 mmol) in THF (48 mL). The crude material was purified by flash column chromatography eluting with ethyl acetate (0-10%) in 40-60 petroleum ether to yield the title compound as a colourless oil (7.82 g, 23.8 mmol, 70%).

(ES, m/z): [M+H]⁺=328.3

### tert-butyl(4-(2-fluoro-5-nitrophenoxy)butoxy)diphenylsilane

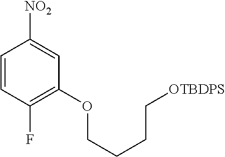

General procedure 22 was applied to 5-fluoro-3-nitrophenol (1.50 g, 9.55 mmol) with 4-((tert-butyldiphenylsilyl)oxy)butan-1-ol (3.45 g, 10.5 mmol), triphenylphosphine (3.26 g, 12.4 mmol) and DIAD (2.32 mL, 12.4 mmol) in THF (24 mL). The crude material was purified by flash column chromatography eluting with ethyl acetate (0-10%) in 40-60 petroleum ether to yield the title compound as a white solid (4.32 g, 9.24 mmol, 97%).

### 1-(2-(4-((tert-butyldiphenylsilyl)oxy)butoxy)-4-nitrophenyl)-4-methylpiperazine

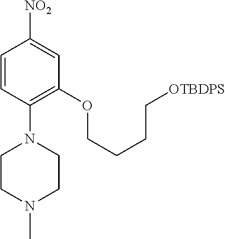

General procedure 11 was applied to tert-butyl(4-(2-fluoro-5-nitrophenoxy)butoxy)diphenylsilane (4.32 g, 9.24 mmol) with N-methylpiperazine (1.12 mL, 10.2 mmol) and potassium carbonate (1.53 mg, 11.1 mmol) in DMSO (15 mL). The crude material was purified by flash column chromatography eluting with ethyl acetate (10-50%) in 40-60 petroleum ether to yield the title compound as a yellow solid (2.98 mg, 5.45 mmol, 59%).

(ES, m/z): [M+H]⁺=547.3

### 3-(4-((tert-butyldiphenylsilyl)oxy)butoxy)-4-(4-methylpiperazin-1-yl)aniline

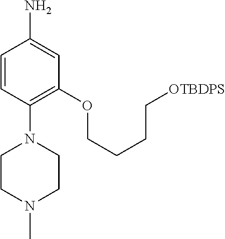

General procedure 12 was applied 1-(2-(4-((tert-butyldiphenylsilyl)oxy)butoxy)-4-nitrophenyl)-4-methylpiperazine (2.98 g, 5.45 mmol) with palladium on carbon (580 mg, 0.545 mmol) in ethanol (55 mL). The crude material was purified by flash column chromatography eluting with methanol (0-10%) in dichloromethane to yield the title compound as a colourless oil (2.11 g, 4.08 mmol, 75%).

(ES, m/z): [M+H]⁺=518.5

### 2-((3-(4-hydroxybutoxy)-4-(4-methylpiperazin-1-yl)phenyl)amino)-5-((triisopropylsilyl)ethynyl)pyrido[2,3-d]pyrimidin-7(8H)-one

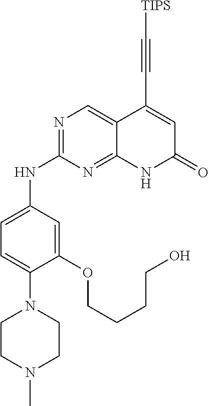

General Procedure 13 was applied to 2-(methylsulfonyl)-5-((triisopropylsilyl)ethynyl)pyrido[2,3-d]pyrimidin-7(8H)-one (1.38 mg, 3.40 mmol) with 3-(4-((tert-butyldiphenylsilyl)oxy)butoxy)-4-(4-methylpiperazin-1-yl)aniline (2.11 g, 3.40 mmol) and trifluoroacetic acid (310 μL, 4.08 mmol) in acetonitrile (17 mL). The crude material was purified by flash column chromatography eluting with methanol (0-20%) in dichloromethane to yield the 2-((3-(4-((tert-butyldiphenylsilyl)oxy)butoxy)-4-(4-methylpiperazin-1-yl)phenyl)amino)-5-((triisopropylsilyl)ethynyl)pyrido[2,3-d]pyrimidin-7(8H)-one, which was stirred at room temperature with trifluoroacetic acid (2.60 mL, 34.0 mmol) in acetonitrile (30 mL) for 1 h. The reaction mixture was concentrated under reduced pressure. The crude material was purified by flash column chromatography eluting with methanol (0-20%) in dichloromethane to yield the title compound as an orange solid (386 mg, 0.640 mmol, 19%).

(ES, m/z): [M+H]⁺=606.6

### 3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one

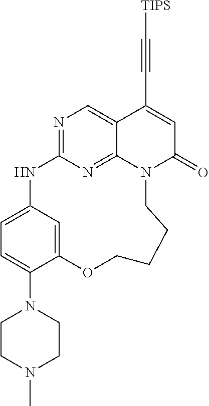

General procedure 20 was applied to 2-((3-(4-hydroxybutoxy)-4-(4-methylpiperazin-1-yl)phenyl)amino)-5-((triisopropylsilyl)ethynyl)pyrido[2,3-d]pyrimidin-7(8H)-one (386 mg, 0.640 mmol) with triphenylphosphine (671 mg, 2.56 mmol) and DIAD (480 μL, 2.56 mmol) in THF (26 mL). The crude material was purified by flash column chromatography eluting with methanol (0-10%) in dichloromethane to yield the title compound as an orange solid (156 mg, 0.270 mmol, 42%).

(ES, m/z): [M+H]⁺=587.5

### 1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-1⁷-one

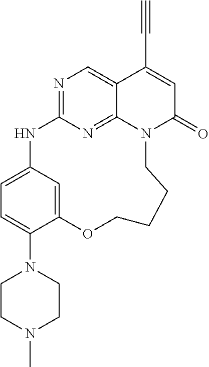 [SMILES: C#Cc1cc(=O)n2c3nc(ncc13)Nc1ccc(N3CCN(C)CC3)c(c1)OCCCC2]

General procedure 15 was applied 3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one (156 mg, 0.270 mmol) with potassium fluoride (309 mg, 5.32 mmol) in DMF (2.7 mL). The crude material was purified by flash column chromatography eluting with methanol (0-20%) in dichloromethane to yield the title compound as an orange solid (84.6 mg, 0.196 mmol, 73%).

(ES, m/z): [M+H]⁺=431.4

¹H NMR (500 MHz, CDCl₃) δ 8.61 (s, 1H), 8.34 (d, J=2.4 Hz, 1H), 6.68 (d, J=8.5 Hz, 1H), 6.47 (dd, J=8.5, 2.3 Hz, 1H), 6.37 (s, 1H), 4.23 (s, 11H), 4.11 (s, 3H), 3.73 (s, 1H), 2.96 (s, 4H), 2.65 (s, 5H), 2.29 (s, 3H), 1.89 (d, J=11.2 Hz, 2H), 1.81 (s, 3H).

### Example 190

### 8-[(2Z)-4-[(tert-Butyldiphenylsilyl)oxy]but-2-en-1-yl]-6-methyl-2-(methylsulfanyl)-5-[2-(triisopropylsilyl)ethynyl]pyrido[2,3-d]pyrimidin-7-one

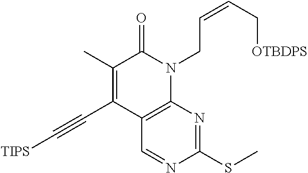

General procedure 18 was applied to 6-methyl-2-(methylsulfanyl)-5-[2-(triisopropylsilyl)ethynyl]-8H-pyrido[2,3-d]pyrimidin-7-one (6.00 g, 15.5 mmol), (2Z)-4-[(tert-butyldiphenylsilyl)oxy]but-2-en-1-ol (10.1 g, 30.9 mmol), triphenylphosphine (4.21 g, 16.0 mmol) and DIAD (6.26 g, 30.9 mmol) in dichloromethane (10 mL). The residue was purified by reverse phase flash column chromatography eluting with acetonitrile (10-50%) in water (0.1% formic acid) to yield the title compound as a white solid (5.70 g, 53%).

### 8-[(2Z)-4-[(tert-Butyldiphenylsilyl)oxy]but-2-en-1-yl]-2-methanesulfonyl-6-methyl-5-[2-(triisopropyisiiyl)ethynyl]pyrido[2,3-d]pyrimidin-7-one

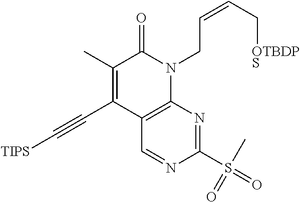

General procedure 10 was applied to 8-[(2Z)-4-[(tert-butyldiphenylsilyl)oxy]but-2-en-1-yl]-6-methyl-2-(methylsulfanyl)-5-[2-(triisopropylsilyl)ethynyl]pyrido[2,3-d]pyrimidin-7-one (5.50 g, 7.90 mmol) and mCPBA (3.82 g, 22.1 mmol) in dichloromethane (55 mL) to yield the title compound as a yellow oil (5.70 g, 99%).

### 8-[(2Z)-4-[(tert-Butyldiphenylsilyl)oxy]but-2-en-1-yl]-2-({3-[(tert-butyldiphenylsilyl)oxy]phenyl}amino)-6-methyl-5-[2-(triisopropylsilyl)ethynyl]pyrido[2,3-d]pyrimidin-7-one

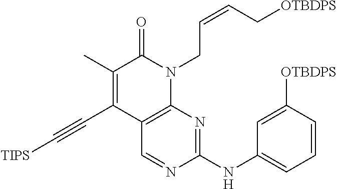

General procedure 13 was applied to 8-[(2Z)-4-[(tert-butyldiphenylsilyl)oxy]but-2-en-1-yl]-2-methanesulfonyl-6-methyl-5-[2-(triisopropylsilyl)ethynyl]pyrido[2,3-d]pyrimidin-7-one (5.70 g, 7.83 mmol), 3-[(tert-butyldiphenylsilyl)oxy]aniline (3.26 g, 9.39 mmol) and trifluoroacetic acid (1.16 mL, 15.6 mmol) in acetonitrile (57 mL), stirred for 14 hours at 80° C. The residue was purified by flash column chromatography, eluting with /dichloromethane (60%) in 40-60 petroleum ether to afford the title compound as a yellow solid (2.00 g, 25%).

### 8-[(2Z)-4-hydroxybut-2-en-1-yl]-2-[(3-hydroxyphenyl)amino]-6-methyl-5-[2-(triisopropylsilyl)ethynyl]pyrido[2,3-d]pyrimidin-7-one

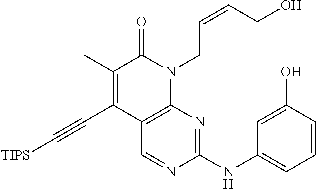

A solution of 8-[(2Z)-4-[(tert-butyldiphenylsilyl)oxy]but-2-en-1-yl]-2-[(3-hydroxyphenyl)amino]-6-methyl-5-[2-(triisopropylsilyl)ethynyl]pyrido[2,3-d]pyrimidin-7-one (2.00 g, 2.64 mmol) in hydrochloric acid in methanol (963 mg, 26.4 mmol) was stirred for 14 hours at 60° C. The resulting mixture was concentrated under reduced pressure. The residue was purified by flash column chromatography, eluting with ethyl acetate (40%) in 40-60 petroleum ether to afford the title compound as a yellow solid (500 mg, 36%).

### (Z)-1⁶-methyl-1⁵-((triisopropylsilyl)ethynyl)-1⁷, 1⁸-dihydro-4-oxa-2-aza-1(2,8)-pyrido[2,3-d]pyrimidina-3(1,3)-benzenacyclooctaphan-6-en-1⁷-one

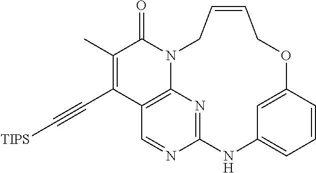

General procedure 20 was applied to 8-[(2Z)-4-hydroxybut-2-en-1-yl]-2-[(3-hydroxyphenyl)amino]-5-[2-(triisopropylsilyl)ethynyl]pyrido[2,3-d]pyrimidin-7-one (280 mg, 0.555 mmol), triphenylphosphine (291 mg, 1.11 mmol) and DIAD (224 mg, 1.110 mmol) in THF (2.0 mL). The residue was purified by flash column chromatography, eluting with ethyl acetate (10%) in 40-60 petroleum ether to afford the title compound as a yellow solid (140 mg, 52%).

### (Z)-1⁵-ethynyl-1⁶-methyl-1⁷,1⁸-dihydro-4-oxa-2-aza-1(2,8)-pyrido[2,3-d]pyrimidina-3(1,3)-benzenacyclooctaphan-6-en-1⁷-one

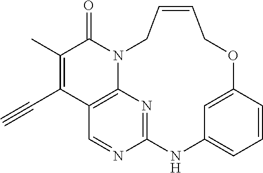 [SMILES: C#Cc1c(C)c(=O)n2c3nc(ncc13)Nc1cccc(c1)OC/C=C\C2]

General procedure potassium fluoride was applied to (Z)-1⁶-methyl-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-6-en-1⁷-one (140 mg, 0.280 mmol) and potassium fluoride (162 mg, 2.80 mmol) in THF (2.0 mL), DMF (2.0 mL) and water (140 uL), stirred for 2 hours at 60° C. The residue was purified by reverse phase flash column chromatography eluting with acetonitrile (10-50%) in water (0.1% trifluoroacetic acid) to yield the title compound as an orange solid (10.3 mg, 11%).

(ES, m/z): [M+H]⁺=345.05

¹H-NMR (400 MHz, DMSO-d₆) δ 10.37 (s, 1H), 9.41 (t, J=2.3 Hz, 1H), 8.78 (s, 1H), 7.11 (t, J=8.0 Hz, 1H), 6.75 (dd, J=7.9, 2.0 Hz, 1H), 6.49 (dd, J=8.1, 2.6 Hz, 1H), 5.66 (t, J=11.2 Hz, 1H), 5.50 (t, J=10.5 Hz, 1H), 5.26 (s, 1H), 5.20-4.99 (m, 3H), 4.60 (d, J=14.3 Hz, 1H), 2.27 (s, 3H).

### Example 191

### (4-Bromobutoxy)(tert-butyl)dimethylsilane

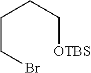 [SMILES: CC(C)(C)[Si](C)(C)OCCCCBr]

To a stirred mixture of 4-bromobutan-1-ol (20.0 g, 131 mmol, 1.0 eq.) and tert-butyldimethylchlorosilane (1.18 g, 7.84 mmol, 1.2 eq.) in dichloromethane (200 mL) was added 1H-imidazole (13.3 g, 196 mmol, 1.5 eq.) in portions at room temperature. The resulting mixture was stirred for 14 hours at room temperature then diluted with water (100 mL) and extracted with ethyl acetate (3×200 mL). The combined organic layers were washed with brine (3×100 mL), dried (Na₂SO₄) and concentrated under reduced pressure- to yield the title compound as a light yellow liquid (30.0 g, 86%).

### tert-Butyl N-(3-(4-[(tert-butyldimethylsilyl)oxy]butoxyphenyl)carbamate

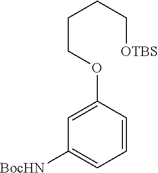 [SMILES: CC(C)(C)OC(=O)Nc1cccc(OCCCCO[Si](C)(C)C(C)(C)C)c1]

To a stirred mixture of (4-bromobutoxy)(tert-butyl)dimethylsilane (15.0 g, 56.1 mmol, 1.0 eq.) and tert-butyl N-(3-hydroxyphenyl)carbamate (11.7 g, 56.1 mmol, 1.0 eq.) in acetonitrile (150 mL) was added potassium carbonate (15.5 g, 112 mmol, 2.0 eq.) in portions at room temperature. The resulting mixture was stirred for 14 hours at 80° C. then diluted with water (100 mL) and extracted with ethyl acetate (3×200 mL). The combined organic layers were washed with brine (3×100 mL), dried (Na₂SO₄) and concentrated under reduced pressure. The residue was purified by flash column chromatography, eluting with ethyl acetate (10%) 40-60 petroleum ether to afford the title compound as a light yellow oil (11.6 g, 52%).

### 3-(4-[(tert-butyldimethylsilyl)oxy]butoxyaniline

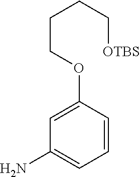 [SMILES: CC(C)(C)[Si](C)(C)OCCCCOc1cccc(N)c1]

A solution of tert-butyl N-(3-(4-[(tert-butyldimethylsilyl)oxy]butoxyphenyl)carbamate (6.00 g, 15.1 mmol) in hexafluoroisopropanol (60 mL) was stirred for 14 hours at 90°. The resulting mixture was concentrated under reduced pressure. The residue was purified by flash column chromatography, eluting with ethyl acetate (18%) in 40-60 petroleum ether to afford the title compound as a light yellow oil (3.24 g, 72%).

### 2-[(3-(4-[(tert-Butyldimethylsilyl)oxy]butoxyphenyl)amino]-6-methyl-5-[2-(triisopropylsilyl) ethynyl]-8H-pyrido[2,3-d]pyrimidin-7-one

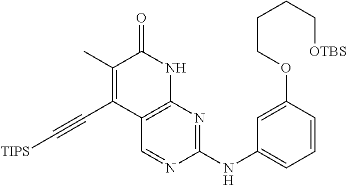

General procedure 13 was applied to 2-methanesulfonyl-6-methyl-5-[2-(triisopropylsilyl)ethynyl]-8H-pyrido[2,3-d]pyrimidin-7-one (2.00 g, 4.76 mmol), 3-(4-[(tert-butyldimethylsilyl)oxy]butoxyaniline (1.69 g, 5.72 mmol) and trifluoroacetic acid (1.09 g, 9.53 mmol) in 2-butanol (200 mL), stirred for 12 hours at 100° C. The residue was purified by reverse phase flash column chromatography eluting with methanol (50-100%) in water (0.1% in ammonium acetate), to yield the title compound as a yellow solid (800 mg, 26%).

### 2-([3-(4-Hydroxybutoxy) phenyl]amino-6-methyl-5-[2-(triisopropylsilyl) ethynyl]-8H-pyrido[2,3-d]pyrimidin-7-one

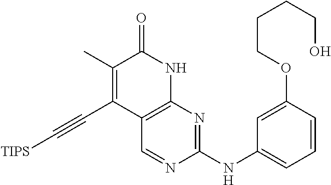

A mixture of 2-[(3-(4-[(tert-butyldimethylsilyl)oxy]butoxyphenyl)amino]-6-methyl-5-[2-(triisopropylsilyl)ethynyl]-8H-pyrido[2,3-d]pyrimidin-7-one (800 mg, 1.260 mmol, 1.0 eq.) in 4M hydrochloric acid in methanol (8.0 mL) was stirred for 14 hours at room temperature. The resulting mixture was concentrated under reduced pressure to yield the title compound as a light yellow solid (400 mg, 61%).

### 1⁶-Methyl-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one

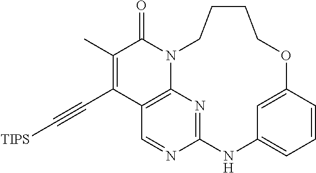

General procedure 20 was applied to 2-([3-(4-hydroxybutoxy)phenyl]amino-6-methyl-5-[2-(triisopropylsilyl)ethynyl]-8H-pyrido[2,3-d]pyrimidin-7-one (400 mg, 0.768 mmol) and triphenylphosphine (806 mg, 3.07 mmol) and DIAD (621 mg, 3.07 mmol) in THF (4.0 mL). The residue was purified by flash column chromatography, eluting with methanol (10%) in dichloromethane to afford the title compound as a light yellow solid (600 mg, 155%).

### 1⁵-ethynyl-1⁶-methyl-1⁷,1⁸-dihydro-4-oxa-2-aza-1(2,8)-pyrido[2,3-d]pyrimidina-3(1,3)-benzenacyclooctaphan-1⁷-one

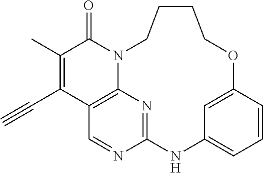 [SMILES: C#Cc1c(C)c(=O)n2c3nc(ncc13)Nc1cccc(c1)OCCCC2]

General procedure 15 was applied to 1⁶-Methyl-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-17-one (300 mg, 0.597 mmol) and potassium fluoride (346 mg, 5.97 mmol) in THF (1.5 mL), DMF (1.5 mL) and water (0.20 mL), stirred for 1 hour at 60° C. The residue was purified by reverse phase flash column chromatography eluting with acetonitrile (10-60%) in water (0.1% formic acid), to yield the title compound as a yellow solid (12.2 mg, 6%).

(ES, m/z): [M+H]⁺=346.95

¹H NMR (300 MHz, DMSO-d₆) δ 10.26 (s, 1H), 8.83-8.63 (m, 2H), 7.14 (t, J=8.0 Hz, 1H), 6.84-6.71 (m, 1H), 6.52 (dd, J=8.5, 2.4 Hz, 1H), 5.24 (s, 1H), 4.28 (s, 4H), 2.24 (s, 3H), 1.93 (s, 4H).

### Example 192

### (1R,3S)-Cyclopentane-1,3-dicarboxylic acid

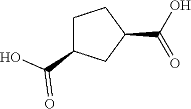 [SMILES: O=C(O)[C@@H]1CC[C@H](C(=O)O)C1]

A solution of norbornene (20.0 g, 212 mmol, 1.0 eq.) in ethyl acetate (20 mL) and acetonitrile (20 mL) was treated with ruthenium trichloride monohydrate (960 mg, 4.25 mmol, 0.02 eq.) followed by the addition of sodium periodate (182 g, 849 mmol, 4.0 eq.) and water (30 mL) dropwise at room temperature. The resulting mixture was stirred for 48 hours at room temperature then extracted with ethyl acetate (3×300 mL). The combined organic layers were washed with water (3×200 mL), dried (Na₂SO₄) and concentrated under reduced pressure to yield the title compound as a white solid (10.0 g, 30%).

### [(1R,3S)-3-(Hydroxymethyl)cyclopentyl]methanol

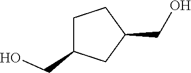

A solution of (1R,3S)-cyclopentane-1,3-dicarboxylic acid (9.00 g, 56.9 mmol, 1.0 eq.) in THF (90 mL) was treated with borane-THF (22.0 g, 256 mmol, 4.5 eq.) at 0° C. The resulting mixture was stirred for 14 hours at room temperature then quenched with water (1.0 mL) at 0° C. The resulting mixture was extracted with ethyl acetate (3×20 mL). The combined organic layers were washed with brine (2×30 mL), dried (Na₂SO₄) and concentrated under reduced pressure to yield the title compound as a yellow oil (7.00 g, 94%).

### [(1S,3R)-3-{[(tert-Butyldiphenylsilyl)oxy]methyl}cyclopentyl]methanol

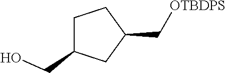

A solution of [(1R,3S)-3-(hydroxymethyl)cyclopentyl]methanol (7 g, 53.769 mmol, 1 eq.) in DMF (70 mL) was treated with Imidazole (2.72 g, 39.942 mmol, 2 eq.) for 5 min at 30° C. under nitrogen atmosphere followed by the addition of TBDPS-Cl (14.78 g, 53.769 mmol, 1 eq.) in portions at room temperature. The resulting mixture was stirred for 14 h at room temperature under N₂ atmosphere. The resulting mixture was extracted with ethyl acetate (2×20 mL). The combined organic layers were washed with NaCl (3×30 mL), dried over anhydrous Na₂SO₄. After filtration, the filtrate was concentrated under reduced pressure. The residue was purified by flash column chromatography, eluting with ethyl acetate in 40-60 petroleum ether (6.8%) to afford (6 g, 30.27%) as a light yellow oil.

### [(1S,3R)-3-{[(tert-Butyldiphenylsilyl)oxy]methyl}cyclopentyl]methyl methanesulfonate

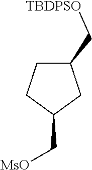

A solution of [(1S,3R)-3-{[(tert-butyldiphenylsilyl)oxy]methyl}cyclopentyl]methanol (1.90 g, 5.15 mmol, 1.0 eq.) in dichloromethane (20 mL) was treated with methanesulfonyl chloride (0.480 mL, 6.19 mmol, 1.2 eq.) followed by the addition of triethylamine (0.930 mL, 6.69 mmol, 1.3 eq.) at 0° C. The resulting mixture was stirred for 2 hours at room temperature then extracted with dichloromethane (2×100 mL). The combined organic layers were washed with saturated aqueous sodium hydrogen carbonate solution (2×100 mL), dried, (Na₂SO₄) and concentrated under reduced pressure to yield the title compound as a light yellow oil (2.20 g, 95%).

### tert-Butyl N-(3-{[(1 S,3R)-3-{[(tert-butyldiphenylsilyl)oxy]methyl}cyclopentyl]methoxy}phenyl)carbamate

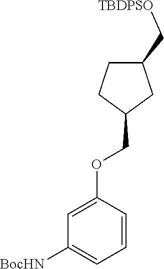

A solution of [(1 S,3R)-3-{[(tert-butyldiphenylsilyl)oxy]methyl}cyclopentyl]methyl methanesulfonate (2.20 g, 4.9 mmol, 1.0 eq.) in DMF (22 mL) was treated with tert-butyl N-(3-hydroxyphenyl)carbamate (1.55 g, 7.39 mmol, 1.5 eq.) followed by the addition of potassium carbonate (1.36 g, 9.85 mmol, 2.0 eq.). The resulting mixture was stirred for 2 hours at room temperature then extracted with dichloromethane (2×30 mL). The combined organic layers were washed with water (2×30 mL), dried (Na₂SO₄) and concentrated under reduced pressure. The residue was purified by flash column chromatography, eluting with ethyl acetate (5%) in 40-60 petroleum ether to afford the title compound as a light yellow oil (1.80 g, 65%).

### 3-{[(1S,3R)-3-{[(tert-butyldiphenylsilyl)oxy]methylcyclopentyl]methoxy}aniline

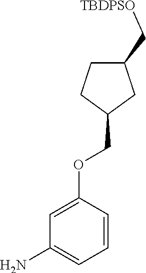

A solution of tert-butyl N-(3-{[(1S,3R)-3-{[(tert-butyldiphenylsilyl)oxy]methyl}cyclopentyl]methoxy}phenyl)carbamate (1.80 g, 3.21 mmol, 1.0 eq.) in 1,1,1,3,3,3-hexafluoropropan-2-ol (18 mL) was stirred for 48 hours at 90° C. The residue was purified by reverse phase flash column chromatography eluting with acetonitrile (30-60%) in water (0.1% trifluoroacetic acid) to yield the title compound as a light yellow oil (1.20 g, 81%).

### 2-[(3-{[(1S,3R)-3-{[(tert-Butyldiphenylsilyl)oxy]methyl}cyclopentyl]methoxy}phenyl)amino]-5-[2-(triisopropylsilyl)ethynyl]-8H-pyrido[2,3-d]pyrimidin-7-one

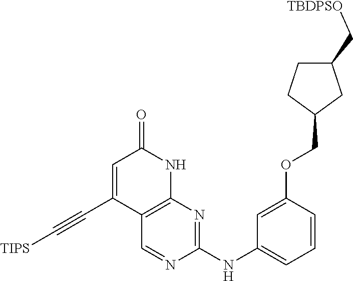

General procedure 13 was applied to 3-{[(1S,3R)-3-{[(tert-butyldiphenylsilyl)oxy]methyl}cyclopentyl]methoxy}aniline (1.20 g, 2.61 mmol) and 2-methanesulfonyl-5-[2-(triisopropylsilyl)ethynyl]-8H-pyrido[2,3-d]pyrimidin-7-one (1.27 g, 3.13 mmol) and trifluoroacetic acid (595 mg, 5.22 mmol) in acetonitrile (12 mL), stirred for 14 hours at 80° C. The precipitated solids were collected by filtration and washed with ethyl acetate (2×5 mL) to yield the title compound as a yellow solid (1.00 g, 49%).

### 2-[(3-{[(1S,3R)-3-(hydroxymethyl)cyclopentyl]methoxy}phenyl)amino]-5-[2-(triisopropylsilyl)ethynyl]-8H-pyrido[2,3-d]pyrimidin-7-one

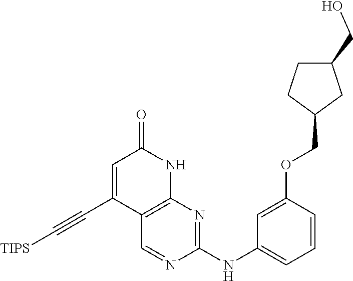

A solution of 2-[(3-{[(1S,3R)-3-{[(tert-butyldiphenylsilyl)oxy]methyl}cyclopentyl]methoxy}phenyl)amino]-5-[2-(triisopropylsilyl)ethynyl]-8H-pyrido[2,3-d]pyrimidin-7-one (1.00 g, 1.27 mmol, 1.0 eq.) in 4M hydrochloric acid in methanol (10 mL) was stirred for 14 hours at 60° C. The precipitated solids were collected by filtration and washed with ethyl acetate (2×5 mL) to yield the title compound as a yellow solid (400 mg, 57%)

### (5¹R,5³S)-3⁵-((triisopropylsilyl)ethynyl)-3⁷,3⁸-dihydro-7-oxa-2-aza-3(2,8)-pyrido[2,3-d]pyrimidina-1(1,3)-benzena-5(1,3)-cyclopentanacycloheptaphan-3⁷-one

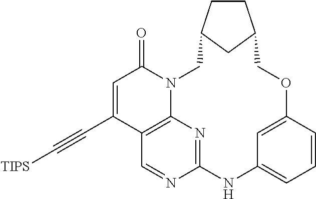

General procedure 20 was applied to 2-[(3-{[(1S,3R)-3-(hydroxymethyl)cyclopentyl]methoxy}phenyl)amino]-5-[2-(triisopropylsilyl)ethynyl]-8H-pyrido[2,3-d]pyrimidin-7-one (400 mg, 0.732 mmol) and triphenylphosphine (767 mg, 2.93 mmol) and DIAD (592 mg, 2.93 mmol) in THF (4.0 mL). The residue was purified by reverse phase flash column chromatography eluting with acetonitrile (50-100%) in water (0.1% formic acid) to yield the title compound as a yellow solid (100 mg, 26%).

### (5¹R,5³S)-3⁵-ethynyl-3⁷,3⁸-dihydro-7-oxa-2-aza-3(2,8)-pyrido[2,3-d]pyrimidina-1(1,3)-benzena-5(1,3)-cyclopentanacycloheptaphan-3⁷-one

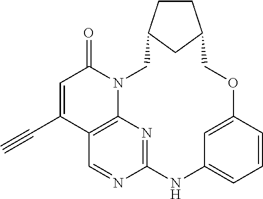 [SMILES: C#Cc1cc(=O)n2c3nc(ncc13)Nc1cccc(c1)OC[C@H]1CC[C@H](C1)C2]

General procedure 15 was applied to (5¹R,5³S)-3⁵-((triisopropylsilyl)ethynyl)-3⁷,3⁸-dihydro-7-oxa-2-aza-3(2,8)-pyrido[2,3-d]pyrimidina-1(1,3)-benzena-5(1,3)-cyclopentanacycloheptaphan-3⁷-one (100 mg, 0.189 mmol) and potassium fluoride (110 mg, 1.89 mmol) in THF (5.0 mL) and water (100 uL), stirred for 4 hours at 60° C. The resulting mixture was washed with acetonitrile and water (1:1 2×30 mL) to yield the title compound as a yellow solid (2.70 mg, 4%).

(ES, m/z): [M+H]⁺=373.00

¹H-NMR (400 MHz, DMSO-d₆): δ 10.42 (s, 1H), 8.83 (s, 2H), 7.15 (t, J=8.1 Hz, 1H), 6.80 (d, J=7.9 Hz, 1H), 6.61 (s, 1H), 6.54 (dd, J=7.9, 2.4 Hz, 1H), 5.08 (s, 1H), 4.25 (s, 2H), 2.03-1.39 (m, 8H), 1.21-1.10 (m, 1H).

### Example 193

### 2-((3-(4-Hydroxybutoxy)phenyl)amino)-5-((triisopropylsilyl)ethynyl)pyrido[2,3-d]pyrimidin-7(8H)-one

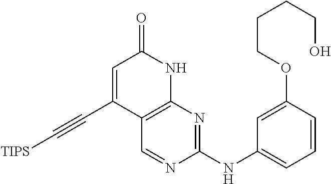

General procedure 13 was applied to 2-methanesulfonyl-5-[2-(triisopropylsilyl)ethynyl]-8H-pyrido[2,3-d]pyrimidin-7-one (3.00 g, 7.39 mmol) and 3-{4-[(tert-butyldimethylsilyl)oxy]butoxy}aniline (4.37 g, 14.8 mmol) and trifluoroacetic acid (1.69 g, 14.8 mmol) in acetonitrile (10 mL), stirred for 14 hours at 80° C. The residue was purified by flash column chromatography, eluting with dichloromethane (60%) in 40-60 petroleum ether to afford the title compound as a yellow solid (550 mg, 12%).

### 1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one

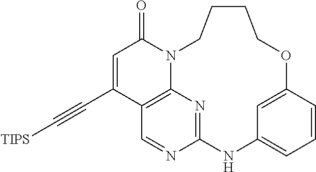

General procedure 20 was applied to 8-[(2Z)-4-hydroxybut-2-en-1-yl]-2-[(3-hydroxyphenyl)amino]-6-methyl-5-[2-(triisopropylsilyl)ethynyl]pyrido[2,3-d]pyrimidin-7-one (280 mg, 0.540 mmol), triphenylphosphine (283 mg, 1.08 mmol) and DIAD (218 mg, 1.08 mmol) in THF (3.0 mL). The residue was purified by flash column chromatography, eluting with ethyl acetate (10%) in 40-60 petroleum ether to yield the title compound as a yellow solid (140 mg, 52%).

### 1⁵-Ethynyl-1⁷,1⁸-dihydro-4-oxa-2-aza-1(2,8)-pyrido[2,3-d]pyrimidina-3(1,3)-benzenacyclooctaphan-1⁷-one

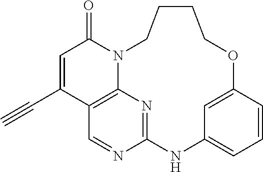 [SMILES: C#Cc1cc(=O)n2c3nc(ncc13)Nc1cccc(c1)OCCCC2]

General procedure 15 was applied to 1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one (150 mg, 0.307 mmol) and potassium fluoride (178 mg, 3.07 mmol) in THF (2.0 mL), DMF (2.0 mL) and water (140 uL), stirred for 2 hours at 60° C. The residue was purified by reverse phase flash column chromatography eluting with acetonitrile (10-50%) in water (0.1% trifluoroacetic acid) to yield the title compound as an orange solid (23.7 mg, 23%).

(ES, m/z): [M+H]⁺=333.20

¹H-NMR (400 MHz, DMSO-d₆) δ 10.37 (s, 1H), 8.80 (s, 1H), 8.61 (d, J=48.3 Hz, 1H), 7.14 (t, J=8.1 Hz, 1H), 6.77 (dt, J=8.0, 1.3 Hz, 1H), 6.58 (s, 1H), 6.55-6.50 (m, 1H), 5.05 (s, 1H), 4.27 (d, J=34.1 Hz, 4H), 1.93 (s, 4H).

### Example 194

### 2-[(3-(4-[(tert-Butyldiphenylsilyl)oxy]butoxy-4-(4-methylpiperazin-1-yl)phenyl)amino]-6-methyl-5-[2-(triisopropylsilyl)ethynyl]-8H-pyrido[2,3-d]pyrimidin-7-one

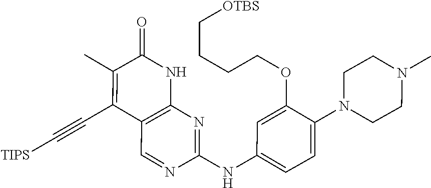

General procedure 13 was applied to 7-cyclopropoxy-2-methanesulfonyl-5-[2-(triisopropylsilyl)ethynyl]pyrido[2,3-d]pyrimidine (50.0 mg, 0.112 mmol) and 3-(4-((tert-butyldimethylsilyl)oxy)butoxy)-4-(4-methylpiperazin-1-yl)aniline (31.2 mg, 0.164 mmol) and trifluoroacetic acid (862 uL, 11.6 mmol) in 2-methyl-2-butanol (1.0 mL), stirred for 12 hours at 80° C. The residue was purified by reverse phase flash column chromatography eluting with acetonitrile (10-50%) in water (0.1% formic acid) to yield the title compound as an orange solid (1.50 mg, 30.14%).

### 2-([3-(4-Hydroxybutoxy)-4-(4-methylpiperazin-1-yl)phenyl]amino-6-methyl-5-[2-(triisopropylsilyl)ethynyl]-8H-pyrido[2,3-d]pyrimidin-7-one

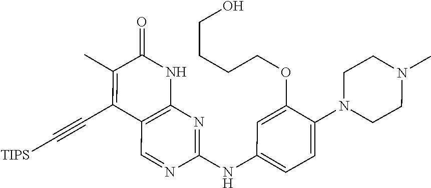

A solution of 2-[(3-(4-[(tert-butyldiphenylsilyl)oxy]butoxy-4-(4-methylpiperazin-1-yl)phenyl)amino]-6-methyl-5-[2-(triisopropylsilyl)ethynyl]-8H-pyrido[2,3-d]pyrimidin-7-one (400 mg, 0.467 mmol, 1.0 eq.) in hydrochloric acid in methanol (4.0 mL) was stirred for 14 hours at room temperature The residue was purified by flash column chromatography, eluting with methanol (5%) dichloromethane to afford the title compound as a light yellow oil (130 mg, 45%).

### 1⁶-methyl-3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one

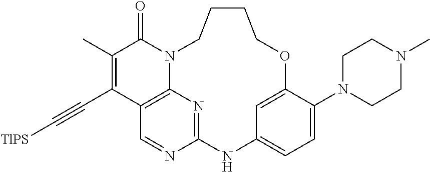

General procedure 20 was applied to 2-([3-(4-hydroxybutoxy)-4-(4-methylpiperazin-1-yl)phenyl]amino-6-methyl-5-[2-(triisopropylsilyl)ethynyl]-8H-pyrido[2,3-d]pyrimidin-7-one (330 mg, 0.533 mmol), with triphenylphosphine (279 mg, 1.06 mmol) and DIAD (215 mg, 1.06 mmol) in THF (5 mL). The residue was purified by flash column chromatography, eluting with methanol (5%) dichloromethane to afford the title compound as a yellow solid (180 mg, 56%).

### 1⁵-Ethynyl-1⁶-methyl-3⁴-(4-methylpiperazin-1-yl)-1⁷,1⁸-dihydro-4-oxa-2-aza-1(2,8)-pyrido [2,3-d]pyrimidina-3(1,3)-benzenacyclooctaphan-1⁷-one

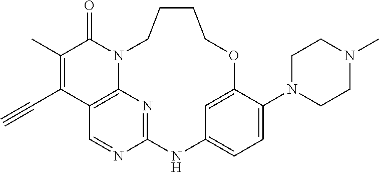 [SMILES: C#Cc1c(C)c(=O)n2c3nc(ncc13)Nc1ccc(N3CCN(C)CC3)c(c1)OCCCC2]

General procedure 15 was applied to 1⁶-methyl-3⁴-(4-methylpiperazin-1-yl)-15-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one (180 mg, 0.300 mmol) and potassium fluoride (174 mg, 3.00 mmol) in THF (2.0 mL) and water (180 μL), stirred for 2 hours at 60° C. The resulting mixture was washed with acetonitrile and water (1:1, 2×30 mL) to yield the title compound as an orange solid (32.7 mg, 24%).

(ES, m/z): [M+H]⁺=445.15

¹H-NMR (400 MHz, DMSO-d₆) δ 10.09 (s, 1H), 8.73 (s, 1H), 8.47 (d, J=2.4 Hz, 1H), 6.79 (d, J=8.5 Hz, 1H), 6.70 (dd, J=8.6, 2.3 Hz, 1H), 5.22 (s, 1H), 4.32 (s, 2H), 4.21 (s, 2H), 2.94 (s, 4H), 2.44 (s, 4H), 2.22 (d, J=6.7 Hz, 6H), 1.91 (d, J=25.3 Hz, 4H).

### Example 195

### (Z)-tert-Butyldphenyl((4-((3-nitrobenzyl)oxy)but-2-en-1-yl)oxy)silane

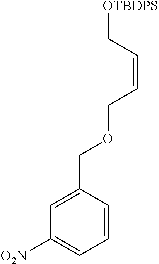

To a stirred solution of (Z)-4-((tert-butyldiphenylsilyl)oxy)but-2-en-1-ol (2.00 g, 6.12 mmol, 1.0 eq.) in THF (20 mL, 0.3 M) at 0° C. was added sodium hydride (490 mg, 12.2 mmol, 2.0 eq.) After 30 minutes at room temperature, benzyl bromide (1.98 g, 9.18 mmol, 1.5 eq.) in THF (10 mL, 0.6 M) was added. The reaction was stirred at room temperature until complete, then poured into water (50 mL). The aqueous layer was extracted with dichloromethane (3×30 mL), the combined organic layers dried (MgSO₄) and concentrated under reduced pressure. The crude material was purified by flash column chromatography eluting with ethyl acetate (0-10%) in 40-60 petroleum ether to yield the title compound as a colourless oil (1.24 g, 2.69 mmol, 44%).

(ES, m/z): [M+H]⁺=463.4

¹H NMR (500 MHz, CDC₃) δ 8.06-8.02 (m, 1H), 7.67-7.62 (m, 1H), 7.62-7.56 (m, 4H), 7.51 (ddq, J=7.6, 1.7, 0.8 Hz, 1H), 7.40 (d, J=7.9 Hz, 1H), 7.37-7.33 (m, 2H), 7.33-7.27 (m, 4H), 5.81-5.68 (m, 1H), 5.55 (dtt, J=11.3, 6.4, 1.7 Hz, 1H), 4.37 (s, 2H), 4.18 (ddt, J=6.2, 1.7, 0.9 Hz, 2H), 3.89 (ddt, J=6.4, 1.7, 0.9 Hz, 2H), 0.96 (s, 9H).

### (Z)-3-(((4-((tert-Butyldiphenylsilyl)oxy)but-2-en-1-yl)oxy)methyl)aniline

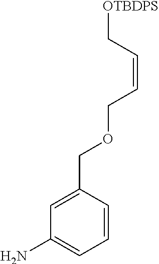

A solution of 1-(2-((5-((tert-butyldiphenylsilyl)oxy)pentyl)oxy)-4-nitrophenyl)-4-methylpiperazine (1.24 g, 2.69 mmol, 1.0 eq.) and iron (750 mg, 13.4 mmol, 5.0 eq.) in saturated aqueous ammonium chloride solution (3.6 mL, 0.8 M) and methanol (7.2 mL, 0.4 M) was stirred at 60° C. for 2 hours. The reaction mixture was filtered through celite, washing with dichloromethane, then extracted with dichloromethane (3×30 mL). The combined organic layers were dried (MgSO₄) and concentrated under reduced pressure. The crude material was purified by flash column chromatography eluting with methanol (0-5%) in dichloromethane to yield the title compound as a purple oil (382 mg, 0.865 mmol, 32%).

(ES, m/z): [M+H]⁺=432.4

¹H NMR (500 MHz, CDC₃) δ 7.77-7.67 (m, 4H), 7.50-7.43 (m, 2H), 7.42-7.36 (m, 4H), 7.12 (t, J=7.7 Hz, 1H), 6.71 (dt, J=7.6, 1.2 Hz, 1H), 6.69-6.64 (m, 2H), 5.85-5.76 (m, 1H), 5.64 (dtt, J=11.3, 6.4, 1.7 Hz, 1H), 4.34 (s, 2H), 4.27 (ddt, J=6.0, 1.7, 0.9 Hz, 2H), 3.92 (ddt, J=6.4, 1.6, 0.9 Hz, 2H), 1.07 (s, 9H).

### (Z)-2-((3-(((4-Hydroxybut-2-en-1-yl)oxy)methyl)phenyl)amino)-5-((triisopropylsilyl)ethynyl)pyrido[2,3-d]pyrimidin-7(8H)-one

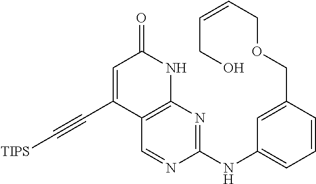

General procedure 13 was applied to 2-(methylsulfonyl)-5-((triisopropylsilyl)ethynyl)pyrido[2,3-d]pyrimidin-7(8H)-one (300 mg, 0.737 mmol), (Z)-3-(((4-((tert-butyldiphenylsilyl)oxy)but-2-en-1-yl)oxy)methyl)aniline (382 mg, 0.382 mmol) and trifluoroacetic acid (86.0 μL, 0.865 mmol) in acetonitrile (7.4 mL). The crude material was purified by flash column chromatography eluting with methanol (0-20%) in dichloromethane to yield the title compound as an orange solid (159 mg, 0.307 mmol, 41%).

(ES, m/z): [M+H]⁺=519.4

¹H NMR (500 MHz, DMSO) δ 10.08 (s, 1H), 8.70 (s, 1H), 7.87 (s, 1H), 7.82 (d, J=8.2 Hz, 1H), 7.29 (t, J=7.8 Hz, 1H), 7.00 (d, J=7.6 Hz, 1H), 6.43 (s, 1H), 5.70-5.61 (m, 1H), 5.60-5.52 (m, 1H), 4.47 (s, 2H), 4.06 (d, J=6.5 Hz, 2H), 3.99 (dd, J=15.2, 6.2 Hz, 2H), 1.26-1.17 (m, 3H), 1.14 (d, J=6.4 Hz, 18H).

### (Z)-1⁵-((Triisopropylsilyl)ethynyl)-1⁷,1⁸-dihydro-5-oxa-2-aza-1(2,8)-pyrido[2,3-d]pyrimidina-3(1,3)-benzenacyclononaphan-7-en-1⁷-one

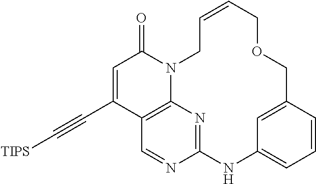

General procedure 20 was applied to (Z)-2-((3-(((4-hydroxybut-2-en-1-yl)oxy)methyl)phenyl)amino)-5-((triisopropylsilyl)ethynyl)pyrido[2,3-d]pyrimidin-7(8H)-one (159 mg, 0.307 mmol), triphenylphosphine (322 mg, 1.23 mmol) and DIAD (230 μL, 1.23 mmol) in THF (13 mL). The crude material was purified by flash column chromatography eluting with methanol (0-20%) in dichloromethane to yield the title compound as an orange solid (85.4 mg, 0.171 mmol, 56%).

(ES, m/z): [M+H]⁺=501.4

¹H NMR (500 MHz, CDC₃) δ 8.85 (d, J=50.2 Hz, 1H), 7.98 (s, 1H), 7.43-7.34 (m, 1H), 7.08-7.01 (m, 2H), 6.70 (d, J=1.4 Hz, 1H), 5.92 (t, J=5.6 Hz, 1H), 5.69-5.63 (m, 1H), 4.95 (s, 2H), 4.77 (d, J=11.6 Hz, 2H), 4.28 (s, 2H), 1.37-1.14 (m, 21H).

### (Z)-1⁵-ethynyl-1⁷,1⁸-dihydro-5-oxa-2-aza-1(2,8)-pyrido[2,3-d]pyrimidina-3(1,3)-benzenacyclononaphan-7-en-1⁷-one

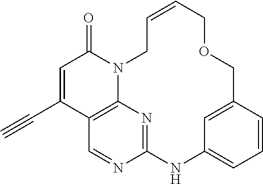 [SMILES: C#Cc1cc(=O)n2c3nc(ncc13)Nc1cccc(c1)COC/C=C\C2]

General procedure 15 was applied to (Z)-1⁵-((triisopropylsilyl)ethynyl)-1⁷,1⁸-dihydro-5-oxa-2-aza-1(2,8)-pyrido[2,3-d]pyrimidina-3(1,3)-benzenacyclononaphan-7-en-17-one (80.0 mg, 0.160 mmol) and potassium fluoride (186 mg, 3.20 mmol) in DMF (2.0 mL). The crude material was purified by flash column chromatography eluting with methanol (0-20%) in dichloromethane to yield the title compound as an orange solid (19.2 mg, 55.7 μmol, 35%).

(ES, m/z): [M+H]⁺=345.2

¹H NMR (500 MHz, CDC₃) δ 8.80 (s, 1H), 7.94 (d, J=2.2 Hz, 1H), 7.28 (t, J=7.7 Hz, 1H), 6.93 (d, J=7.6 Hz, 1H), 6.89 (dd, J=7.8, 2.2 Hz, 1H), 6.60 (s, 1H), 5.85 (s, 1H), 5.61 (dt, J=11.6, 6.2 Hz, 1H), 4.88 (s, 2H), 4.66 (s, 2H), 4.22 (s, 2H), 3.63 (s, 1H).

### Example 196

### tert-Butyl(3-((2-fluoro-5-nitrobenzyl)oxy)propoxy)dimethylsilane

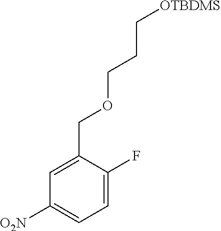 [SMILES: CC(C)(C)[Si](C)(C)OCCCOCc1cc([N+](=O)[O-])ccc1F]

To a stirred solution of 3-((tert-butyldimethylsilyl)oxy)propan-1-ol (1.00 g, 5.25 mmol, 1.0 eq.) in THF (45 mL) at 0° C. was added sodium hydride (420 mg, 10.5 mmol, 2.0 eq.). After 30 minutes, 2-fluoro-5-nitrobenzyl bromide (1.84 g, 7.88 mmol, 1.5 eq.) was added and the reaction was stirred at room temperature overnight. The reaction was quenched with water (50 mL) and extracted with dichloromethane (3×50 mL). The combined organic layers were dried (MgSO₄) and concentrated under reduced pressure. The crude material was purified by flash column chromatography eluting with ethyl acetate (0-10%) in 40-60 petroleum ether to yield the title compound as a colourless oil (874 mg, 2.55 mmol, 48%).

¹H NMR (500 MHz, CDC₃) δ 8.20 (d, J=2.8 Hz, 1H), 8.16 (dd, J=9.0, 2.8 Hz, 1H), 6.93 (d, J=9.1 Hz, 1H), 4.47 (s, 2H), 4.21 (t, J=6.0 Hz, 2H), 3.81 (t, J=5.9 Hz, 2H), 2.03 (p, J=5.9 Hz, 2H), 0.84 (s, 9H), −0.00 (s, 6H).

### 1-(2-((3-((tert-Butyldimethylsilyl)oxy)propoxy)methyl)-4-nitrophenyl)-4-methylpiperazine

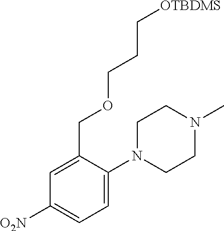 [SMILES: CN1CCN(c2ccc([N+](=O)[O-])cc2COCCCO[Si](C)(C)C(C)(C)C)CC1]

General procedure 11 was applied to tert-butyl(3-((2-fluoro-5-nitrobenzyl)oxy)propoxy)dimethylsilane (874 mg, 2.55 mmol), potassium carbonate (423 mg, 3.06 mmol) and N-methylpiperazine (0.300 mL, 2.80 mmol) in DMSO (4.3 mL). The crude material was purified by flash column chromatography eluting with methanol (0-10%) in dichloromethane to yield the title compound as an orange solid (1.03 g, 2.43 mmol, 95%).

(ES, m/z): [M+H]⁺=424.4

¹H NMR (500 MHz, CDC₃) δ 8.27 (d, J=2.9 Hz, 1H), 8.10 (dd, J=9.0, 2.9 Hz, 1H), 6.88 (d, J=9.1 Hz, 1H), 4.14 (t, J=6.1 Hz, 2H), 3.78 (t, J=5.9 Hz, 2H), 3.53 (s, 2H), 2.52 (s, 4H), 2.30 (s, 3H), 2.00 (p, J=6.0 Hz, 2H), 1.77 (s, 4H), 0.84 (s, 9H), 0.00 (s, 6H).

### 3-((3-((tert-Butyldimethylsilyl)oxy)propoxy)methyl)-4-(4-methylpiperazin-1-yl)aniline

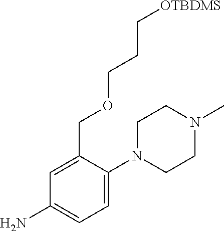 [SMILES: CN1CCN(c2ccc(N)cc2COCCCO[Si](C)(C)C(C)(C)C)CC1]

General procedure 12 was applied to 1-(2-((3-((tert-butyldimethylsilyl)oxy)propoxy)methyl)-4-nitrophenyl)-4-methylpiperazine (1.03 g, 2.43 mmol) and palladium on carbon (259 mg, 0.243 mmol) in ethanol (24 mL). The crude material was purified by flash column chromatography eluting with methanol (0-20%) in dichloromethane to yield the title compound as a purple oil (489 mg, 1.24 mmol, 51%).

¹H NMR (500 MHz, CDC₃) δ 6.78 (s, 1H), 6.68 (d, J=8.5 Hz, 1H), 6.54 (dd, J=8.5, 2.9 Hz, 1H), 3.93 (t, J=6.2 Hz, 2H), 3.74 (t, J=6.0 Hz, 2H), 3.59 (s, 2H), 2.67 (s, 8H), 2.36 (s, 3H), 1.90 (p, J=6.2 Hz, 2H), 0.84 (s, 9H), 0.00 (s, 6H).

### 2-((3-((3-Hydroxypropoxy)methyl)-4-(4-methylpiperazin-1-yl)phenyl)amino)-5-((triisopropylsilyl)ethynyl)pyrido[2,3-d]pyrimidin-7(8H)-one

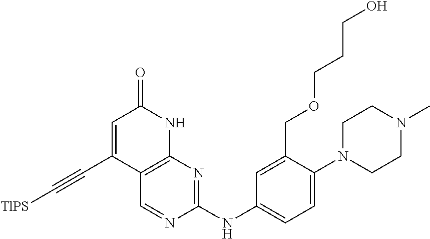

General procedure 13 was applied to 2-(methylsulfonyl)-5-((triisopropylsilyl)ethynyl)pyrido[2,3-d]pyrimidin-7(8H)-one (421 mg, 1.03 mmol), 3-((3-((tert-butyldimethylsilyl)oxy)propoxy)methyl)-4-(4-methylpiperazin-1-yl)aniline (489 mg, 1.24 mmol) and trifluoroacetic acid (95.0 μL, 1.24 mmol) in acetonitrile (10 mL). The crude material was purified by flash column chromatography eluting with methanol (0-20%) in dichloromethane, followed by trituration with ethyl acetate to yield the title compound as an orange solid (439 mg, 0.727 mmol, 70%).

(ES, m/z): [M+H]⁺=605.5

### 3⁴-(4-Methylpiperazin-1-yl)-1⁵-((triisopropylsilyl)ethynyl)-1⁷,1⁸-dihydro-5-oxa-2-aza-1(2,8)-pyrido[2,3-d]pyrimidina-3(1,3)-benzenacyclooctaphan-1⁷-one

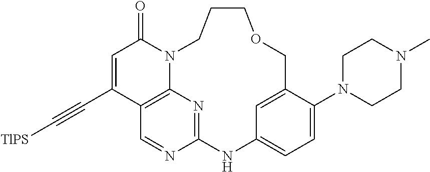

General procedure 20 was applied to 2-((3-((3-hydroxypropoxy)methyl)-4-(4-methylpiperazin-1-yl)phenyl)amino)-5-((triisopropylsilyl)ethynyl)pyrido[2,3-d]pyrimidin-7(8H)-one (435 mg, 0.720 mmol), triphenylphosphine (756 mg, 2.88 mmol) and DIAD (0.540 mL, 2.88 mmol) in THF (30 mL). The crude material was purified by flash column chromatography eluting with methanol (0-20%) in dichloromethane to yield the title compound as an orange solid (159 mg, 0.213 mmol, 29%).

(ES, m/z): [M+H]⁺=587.9

### 1⁵-Ethynyl-34-(4-methylpiperazin-1-yl)-1⁷, 1⁸-dihydro-5-oxa-2-aza-1(2,8)-pyrido[2,3-d]pyrimidina-3(1,3)-benzenacyclooctaphan-1⁷-one

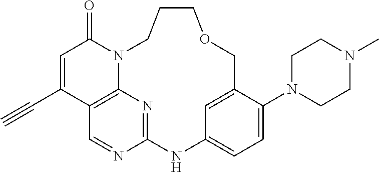 [SMILES: C#Cc1cc(=O)n2c3nc(ncc13)Nc1ccc(N3CCN(C)CC3)c(c1)COCCC2]

General procedure 15 was applied to 3⁴-(4-methylpiperazin-1-yl)-1⁵-((triisopropylsilyl)ethynyl)-1⁷,1⁸-dihydro-5-oxa-2-aza-1(2,8)-pyrido[2,3-d]pyrimidina-3(1,3)-benzenacyclooctaphan-1⁷-one (159 mg, 0.213 mmol) and potassium fluoride (315 mg, 5.43 mmol) in DMF (2.7 mL). The crude material was purified by flash column chromatography eluting with methanol (0-20%) in dichloromethane to yield the title compound as an orange solid (15.0 mg).

(ES, m/z): [M+H]⁺=431.6

¹H NMR (500 MHz, CDCl: CD₃OH (3:1)) δ 8.70 (s, 1H), 7.64-7.58 (m, 1H), 7.10 (d, J=2.6 Hz, 1H), 6.72 (d, J=8.8 Hz, 1H), 6.44 (s, 1H), 4.28 (t, J=8.1 Hz, 2H), 3.84 (t, J=6.4 Hz, 2H), 3.73 (s, 1H), 3.35 (s, 2H), 2.44 (br, 8H), 2.22 (s, 3H), 2.00 (t, J=8.1 Hz, 2H).

### Example 197

### N-(2-((tert-Butyldimethylsilyl)oxy)ethyl)-2-(2-fluoro-5-nitrophenyl)acetamide

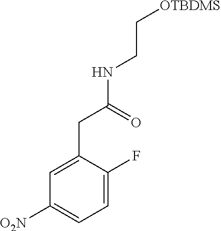 [SMILES: CC(C)(C)[Si](C)(C)OCCNC(=O)Cc1cc([N+](=O)[O-])ccc1F]

To a stirred solution of 2-fluoro-5-phenylacetic acid (1.00 g, 5.02 mmol, 1.0 eq.) in dichloromethane (40 mL) was added HATU (1.32 g, 7.53 mmol, 1.5 eq.) followed by 2-((tert-butyldimethylsilyl)oxy)ethan-1-amine (1.32 g, 7.53 mmol, 1.5 eq.) and diisopropylethylamine (1.78 mL, 10.0 mmol, 2.0 eq.) The reaction was stirred at room temperature overnight, then quenched with water (50 mL) and the organic layer separated. The organic layer was dried (MgSO₄) and concentrated under reduced pressure. The crude material was purified by flash column chromatography eluting with ethyl acetate (0-10%) in 40-60 petroleum ether to yield the title compound as a colourless oil (1.56 g, 4.39 mmol, 87%).

(ES, m/z): [M+H]⁺=357.3

¹H NMR (500 MHz, CDC₃) δ 8.25 (dd, J=6.3, 2.8 Hz, 1H), 8.16 (ddd, J=9.0, 4.4, 2.8 Hz, 1H), 7.18 (d, J=8.8 Hz, 1H), 5.92 (s, 1H), 3.66-3.60 (m, 4H), 3.36 (q, J=5.4 Hz, 2H), 0.82 (s, 9H), 0.00 (s, 6H).

### N-(2-Hydroxyethyl)-2-(2-(4-methylpiperazin-1-yl)-5-nitrophenyl)acetamide

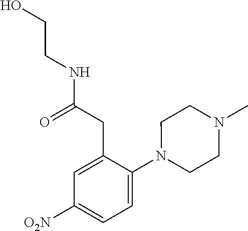 [SMILES: CN1CCN(c2ccc([N+](=O)[O-])cc2CC(=O)NCCO)CC1]

General procedure 11 was applied to N-(2-((tert-butyldimethylsilyl)oxy)ethyl)-2-(2-fluoro-5-nitrophenyl)acetamide (1.56 g, 4.39 mmol), potassium carbonate (728 mg, 5.27 mmol) and N-methylpiperazine (0.530 mL, 4.83 mmol) in DMSO (7.0 mL). The crude material was purified by flash column chromatography eluting with methanol (0-10%) in dichloromethane to yield the title compound as an orange solid (932 mg, 2.89 mmol, 66%).

(ES, m/z): [M+H]⁺=323.2

### N-(2-((tert-Butyldimethylsilyl)oxy)ethyl)-2-(2-(4-methylpiperazin-1-yl)-5-nitrophenyl)acetamide

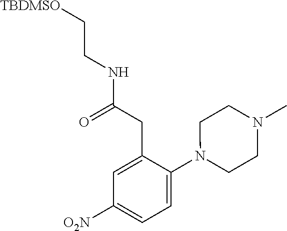 [SMILES: CN1CCN(c2ccc([N+](=O)[O-])cc2CC(=O)NCCO[Si](C)(C)C(C)(C)C)CC1]

A solution of N-(2-hydroxyethyl)-2-(2-(4-methylpiperazin-1-yl)-5-nitrophenyl)acetamide (932 mg, 2.89 mmol, 1.0 eq.), tert-butyldimethylsilylchloride (523 mg, 3.47 mmol, 1.2 eq.) and imidazole (492 mg, 7.23 mmol, 2.5 eq.) in DMF (3.0 mL) was stirred at room temperature overnight. The reaction mixture was quenched with water (30 mL) and extracted with dichloromethane (3×30 mL). The combined organic layers were dried (MgSO4) and concentrated under reduced pressure. The crude material was purified by flash column chromatography eluting with methanol (0-10%) in dichloromethane to yield the title compound as a yellow solid (567 mg, 1.30 mmol, 45%).

(ES, m/z): [M+H]⁺=437.4

¹H NMR (500 MHz, CDC₃) δ 8.18 (dq, J=6.0, 2.7 Hz, 2H), 7.26 (d, J=9.5 Hz, 1H), 5.97 (s, 1H), 3.68 (s, 2H), 3.64 (dd, J=5.6, 4.7 Hz, 2H), 3.37 (q, J=5.3 Hz, 2H), 3.21 (s, 4H), 2.88 (s, 4H), 2.57 (s, 3H), 0.80 (s, 9H), −0.00 (s, 6H).

### 2-(5-Amino-2-(4-methylpiperazin-1-yl)phenyl)-N-(2-((tert-butyidimethylsilyl)oxy)ethyl)acetamide

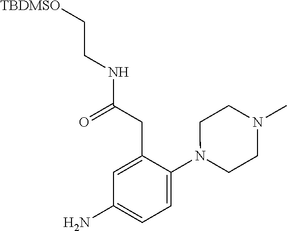 [SMILES: CN1CCN(c2ccc(N)cc2CC(=O)NCCO[Si](C)(C)C(C)(C)C)CC1]

General procedure 12 was applied to N-(2-((tert-butyldimethylsilyl)oxy)ethyl)-2-(2-(4-methylpiperazin-1-yl)-5-nitrophenyl)acetamide (567 mg, 1.30 mmol) and palladium on carbon (138 mg, 0.130 mmol) in acetic acid (1.3 mL) and ethanol (12 mL). The crude material was purified by flash column chromatography eluting with methanol (0-20%) in dichloromethane to yield the title compound as a purple oil (529 mg, 1.30 mmol, 100%).

(ES, m/z): [M+H]⁺=407.4

¹H NMR (500 MHz, CDC₃) δ 7.11 (d, J=8.4 Hz, 1H), 6.64 (dd, J=8.4, 2.8 Hz, 1H), 6.60 (d, J=2.7 Hz, 1H), 3.62 (t, J=5.2 Hz, 2H), 3.55 (s, 2H), 3.32 (q, J=5.3 Hz, 2H), 3.03 (d, J=29.7 Hz, 8H), 2.63 (s, 3H), 0.83 (s, 9H), −0.00 (s, 6H).

### N-(2-Hydroxyethyl)-2-(2-(4-methylpiperazin-1-yl)-5-((7-oxo-5-((triisopropylsilyl)ethynyl)-7,8-dihydropyrido[2,3-d]pyrimidin-2-yl)amino)phenyl)acetamide

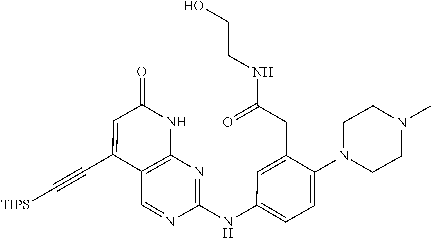

General procedure 13 was applied to 2-(methylsulfonyl)-5-((triisopropylsilyl)ethynyl)pyrido[2,3-d]pyrimidin-7(8H)-one (440 mg, 1.08 mmol), 2-(5-amino-2-(4-methylpiperazin-1-yl)phenyl)-N-(2-((tert-butyldimethylsilyl)oxy)ethyl)acetamide (529 mg, 1.30 mmol) and trifluoroacetic acid (0.100 mL, 1.30 mmol) in acetonitrile (11 mL). The crude material was purified by flash column chromatography eluting with methanol (0-20%) in dichloromethane, followed by trituration with ethyl acetate to yield the title compound as an orange solid (316 mg, 0.511 mmol, 47%).

(ES, m/z): [M+H]⁺=618.5

### 3⁴-(4-Methylpiperazin-1-yl)-1⁵-((triisopropylsilyl)ethynyl)-1⁷,1⁸-dihydro-2,6-diaza-1(2,8)-pyrido[2,3-d]pyrimidina-3(1,3)-benzenacyclooctaphane-1⁷,5-dione

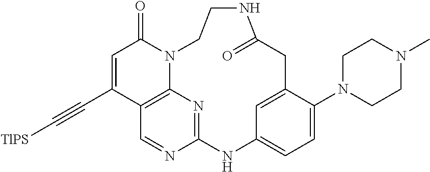

General procedure 20 was applied to N-(2-hydroxyethyl)-2-(2-(4-methylpiperazin-1-yl)-5-((7-oxo-5-((triisopropylsilyl)ethynyl)-7,8-dihydropyrido[2,3-d]pyrimidin-2-yl)amino)phenyl)acetamide (316 mg, 0.511 mmol), triphenylphosphine (537 mg, 2.05 mmol) and DIAD (0.380 mL, 2.05 mmol) in THF (21 mL). The crude material was purified by flash column chromatography eluting with methanol (0-20%) in dichloromethane to yield the title compound as an orange solid (131 mg, 0.219 mmol, 43%).

(ES, m/z): [M+H]⁺=600.5

### 1⁵-Ethynyl-3⁴-(4-methylpiperazin-1-yl)-1⁷,1⁸-dihydro-2,6-diaza-1(2,8)-pyrido[2,3-d]pyrimidina-3(1,3)-benzenacyclooctaphane-1⁷,5-dione

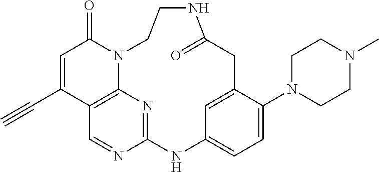 [SMILES: C#Cc1cc(=O)n2c3nc(ncc13)Nc1ccc(N3CCN(C)CC3)c(c1)CC(=O)NCC2]

General procedure 15 was applied to 3⁴-(4-methylpiperazin-1-yl)-1⁵-((triisopropylsilyl)ethynyl)-1⁷,1⁸-dihydro-2,6-diaza-1(2,8)-pyrido[2,3-d]pyrimidina-3(1,3)-benzenacyclooctaphane-1⁷,5-dione (131 mg, 0.219 mmol) and potassium fluoride (254 mg, 4.37 mmol) in DMF (2.2 mL). The crude material was purified by flash column chromatography eluting with methanol (0-20%) in dichloromethane to yield the title compound as an orange solid (30.3 mg, 68.6 μmol, 31%).

(ES, m/z): [M+H]⁺=444.4

### Example 198

### N-(4-((tert-Butyldimethylsilyl)oxy)butyl)-2-fluoro-5-nitroaniline

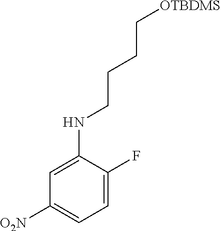 [SMILES: CC(C)(C)[Si](C)(C)OCCCCNc1cc([N+](=O)[O-])ccc1F]

To a stirred solution of 2-fluoro-5-nitroaniline (1.32 g, 8.49 mmol, 1.0 eq.), 4-((tert-butyldimethylsilyl)oxy)butanal (1.72 g, 8.49 mmol, 1.0 eq.) and acetic acid (0.700 mL, 12.7 mmol, 1.5 eq.) in methanol (28 mL) at 0° C. was added sodium cyanoborohydride (1.07 g, 17.0 mmol, 2.0 eq.). The reaction was stirred at room temperature overnight, then quenched with water (50 mL). The aqueous layer was extracted with ethyl acetate (3×50 mL). The combined organic layers were dried (MgSO₄) and concentrated under reduced pressure. The crude material was purified by flash column chromatography eluting with ethyl acetate (0-10%) in 40-60 petroleum ether to yield the title compound as a colourless oil (927 mg, 2.71 mmol, 32%).

(ES, m/z): [M+H]⁺=343.3

¹H NMR (500 MHz, CDC₃) δ 7.49-7.40 (m, 2H), 6.97 (dd, J=10.6, 8.7 Hz, 1H), 3.62 (t, J=6.1 Hz, 2H), 3.18 (t, J=7.0 Hz, 2H), 1.75-1.66 (m, 2H), 1.63-1.54 (m, 2H), 0.83 (s, 9H), −0.00 (s, 6H).

### N-(4-((tert-Butyldimethylsilyl)oxy)butyl)-2-(4-methylpiperazin-1-yl)-5-nitroaniline

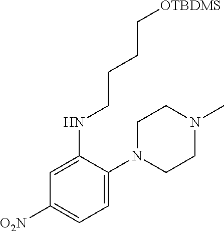 [SMILES: CN1CCN(c2ccc([N+](=O)[O-])cc2NCCCCO[Si](C)(C)C(C)(C)C)CC1]

General procedure 11 was applied to N-(4-((tert-butyldimethylsilyl)oxy)butyl)-2-fluoro-5-nitroaniline 927 mg, 2.71 mmol), lithium carbonate (240 mg, 3.25 mmol) and N-methylpiperazine (0.330 mL, 2.98 mmol) in acetonitrile (4.5 mL), with heating to 70° C. until complete. The crude material was purified by flash column chromatography eluting with methanol (0-10%) in dichloromethane to yield the title compound as an orange solid (818 mg, 1.94 mmol, 71%).

(ES, m/z): [M+H]⁺=423.3

### N¹-(4-((tert-Butyldimethylsilyl)oxy)butyl)-6-(4-methylpiperazin-1-yl)benzene-1,3-diamine

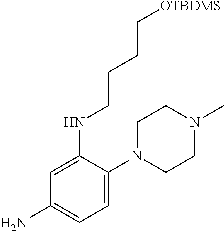 [SMILES: CN1CCN(c2ccc(N)cc2NCCCCO[Si](C)(C)C(C)(C)C)CC1]

General procedure 12 was applied to N-(4-((tert-butyldimethylsilyl)oxy)butyl)-2-(4-methylpiperazin-1-yl)-5-nitroaniline (818 mg, 1.94 mmol) and palladium on carbon (206 mg, 0.194 mmol) in ethanol (19 mL). The crude material was purified by flash column chromatography eluting with methanol (0-20%) in dichloromethane to yield the title compound as a purple oil (605 g, 1.54 mmol, 80%).

(ES, m/z): [M+H]⁺=393.4

¹H NMR (500 MHz, CDC₃) δ 7.63-7.53 (m, 5H), 7.43-7.35 (m, 1H), 7.33-7.27 (m, 4H), 6.78 (d, J=8.1 Hz, 1H), 5.93 (dd, J=8.1, 2.5 Hz, 1H), 5.88 (d, J=2.5 Hz, 1H), 3.66 (t, J=6.0 Hz, 2H), 3.00 (t, J=6.7 Hz, 2H), 2.95-2.76 (m, 8H), 2.38 (s, 3H), 1.72-1.54 (m, 4H), 0.99 (s, 9H).

### 2-((3-((4-Hydroxybutyl)amino)-4-(4-methylpiperazin-1-yl)phenyl)amino)-5-((triisopropylsilyl)ethynyl)pyrido[2,3-d]pyrimidin-7(8H)-one

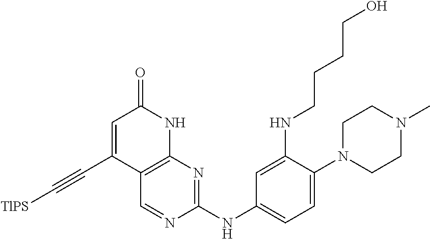

General procedure 13 was applied to 2-(methylsulfonyl)-5-((triisopropylsilyl)ethynyl)pyrido[2,3-d]pyrimidin-7(8H)-one (626 mg, 1.54 mmol), N¹-(4-((tert-butyldimethylsilyl)oxy)butyl)-6-(4-methylpiperazin-1-yl)benzene-1,3-diamine (605 mg, 1.54 mmol) and trifluoroacetic acid (0.130 mL, 1.69 mmol) in acetonitrile (15 mL). The crude material was purified by flash column chromatography eluting with methanol (0-20%) in dichloromethane to yield the title compound as an orange solid (432 mg, 0.716 mmol, 46%).

(ES, m/z): [M+H]⁺=604.5

### 3⁴-(4-Methylpiperazin-1-yl)-1⁵-((triisopropylsilyl)ethynyl)-1⁷,1⁸-dihydro-2,4-diaza-1(2,8)-pyrido[2,3-d]pyrimidina-3(1,3)-benzenacyclooctaphan-1⁷-one

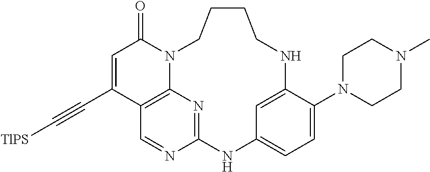

General procedure 20 was applied to 2-((3-((4-hydroxybutyl)amino)-4-(4-methylpiperazin-1-yl)phenyl)amino)-5-((triisopropylsilyl)ethynyl)pyrido[2,3-d]pyrimidin-7(8H)-one (428 mg, 0.710 mmol), triphenylphosphine (745 mg, 2.84 mmol) and DIAD (0.530 mL, 2.84 mmol) in THF (30 mL). The crude material was purified by flash column chromatography eluting with methanol (0-20%) in dichloromethane to yield the title compound as an orange solid (160 mg, 0.274 mmol, 38%).

(ES, m/z): [M+H]⁺=586.6

### 1⁵-Ethynyl-3⁴-(4-methylpiperazin-1-yl)-1⁷, 1⁸-dihydro-2,4-diaza-1(2,8)-pyrido[2,3-d]pyrimidina-3(1,3)-benzenacyclooctaphan-1⁷-one

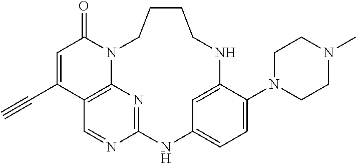 [SMILES: C#Cc1cc(=O)n2c3nc(ncc13)Nc1ccc(N3CCN(C)CC3)c(c1)NCCCC2]

General procedure 15 was applied to 3⁴-(4-methylpiperazin-1-yl)-1⁵-((triisopropylsilyl)ethynyl)-1⁷,1⁸-dihydro-2,4-diaza-1(2,8)-pyrido[2,3-d]pyrimidina-3(1,3)-benzenacyclooctaphan-1⁷-one (56.0 mg, 95.7 μmol) and potassium fluoride (111 mg, 1.91 mmol) in DMF (1.0 mL). The crude material was purified by flash column chromatography eluting with methanol (0-20%) in dichloromethane to yield the title compound as an orange solid (24.3 mg, 57.7 μmol, 59%).

(ES, m/z): [M+H]⁺=430.3

¹H NMR (500 MHz, DMSO) δ 12.20 (s, 1H), 8.70 (s, 1H), 7.35 (s, 1H), 7.19 (t, J=2.9 Hz, 1H), 6.86 (d, J=8.6 Hz, 1H), 6.42 (s, 1H), 5.04 (s, 1H), 3.26 (d, J=6.2 Hz, 4H), 2.90 (br, 4H), 2.51 (br, 4H), 2.30 (s, 3H), 1.89-1.83 (m, 4H).

### Example 199

### 3-((tert-Butyldimethylsilyl)oxy)-N-(2-fluoro-5-nitrobenzyl)propan-1-amine

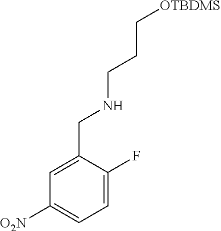 [SMILES: CC(C)(C)[Si](C)(C)OCCCNCc1cc([N+](=O)[O-])ccc1F]

To a stirred solution of 2-fluoro-5-nitrobenzaldehyde (1.01 g, 5.97 mmol, 1.0 eq.), 3-((tert-butyldimethylsilyl)oxy)propan-1-amine (1.13 g, 5.97 mmol, 1.0 eq.) and sodium acetate (735 mg, 8.96 mmol, 1.5 eq.) in methanol (20 mL) was added sodium cyanoborohydride (756 mg, 11.9 mmol, 2.0 eq.). The reaction was stirred at room temperature overnight, then quenched with water (50 mL). The aqueous layer was extracted with dichloromethane (3×50 mL). The combined organic layers were dried (MgSO₄) and concentrated under reduced pressure. The crude material was purified by flash column chromatography eluting with ethyl acetate (0-10%) in 40-60 petroleum ether to yield the title compound as a colourless oil (1.30 g, 3.80 mmol, 63%).

(ES, m/z): [M+H]⁺=343.2

¹H NMR (500 MHz, CDC₃) δ 8.31 (dd, J=6.2, 2.9 Hz, 1H), 8.11 (ddd, J=9.0, 4.4, 2.9 Hz, 1H), 7.13 (t, J=8.9 Hz, 1H), 3.86 (s, 2H), 3.67 (t, J=5.9 Hz, 2H), 2.71 (t, J=6.7 Hz, 2H), 1.75-1.69 (m, 2H), 0.82 (s, 9H), 0.00 (s, 6H).

### 3-((tert-Butyldimethylsilyl)oxy)-N-(2-(4-methylpiperazin-1-yl)-5-nitrobenzyl)propan-1-amine

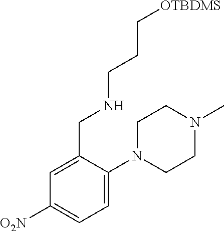 [SMILES: CN1CCN(c2ccc([N+](=O)[O-])cc2CNCCCO[Si](C)(C)C(C)(C)C)CC1]

General procedure 11 was applied to 3-((tert-butyldimethylsilyl)oxy)-N-(2-fluoro-5-nitrobenzyl)propan-1-amine (1.30 g, 3.80 mmol), lithium carbonate (337 mg, 4.56 mmol) and N-methylpiperazine (0.460 mL, 4.18 mmol) in acetonitrile (6.3 mL). The crude material was purified by flash column chromatography eluting with methanol (0-10%) in dichloromethane to yield the title compound as an orange solid (773 mg, 1.83 mmol, 48%).

(ES, m/z): [M+H]⁺=423.4

¹H NMR (500 MHz, CDC₃) δ 8.26 (d, J=2.7 Hz, 1H), 8.03 (dd, J=8.9, 2.8 Hz, 1H), 7.00 (d, J=8.9 Hz, 1H), 3.76 (s, 2H), 3.67 (t, J=6.1 Hz, 2H), 3.06 (t, J=4.9 Hz, 4H), 2.68 (t, J=6.8 Hz, 2H), 2.55 (s, 4H), 2.33 (s, 3H), 1.69 (p, J=6.5 Hz, 2H), 0.83 (s, 9H), 0.00 (s, 6H).

### 3-(((3-((tert-Butyldimethylsilyl)oxy)propyl)amino)methyl)-4-(4-methylpiperazin-1-yl)aniline

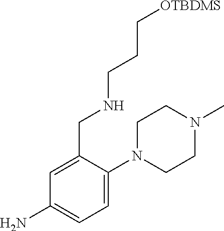 [SMILES: CN1CCN(c2ccc(N)cc2CNCCCO[Si](C)(C)C(C)(C)C)CC1]

General procedure 12 was applied to 3-((tert-butyldimethylsilyl)oxy)-N-(2-(4-methylpiperazin-1-yl)-5-nitrobenzyl)propan-1-amine (773 mg, 1.83 mmol) and palladium on carbon (195 mg, 0.183 mmol) in ethanol (18 mL). The crude material was purified by flash column chromatography eluting with methanol (0-20%) in dichloromethane to yield the title compound as a purple oil (622 g, 1.59 mmol, 87%).

(ES, m/z): [M+H]⁺=393.3

¹H NMR (500 MHz, CDC₃) δ 6.96 (d, J=8.4 Hz, 1H), 6.61 (d, J=2.7 Hz, 1H), 6.54 (dd, J=8.4, 2.8 Hz, 1H), 3.75 (s, 2H), 3.65 (t, J=6.2 Hz, 2H), 3.51 (s, 2H), 2.84 (t, J=4.8 Hz, 4H), 2.67 (t, J=7.1 Hz, 2H), 2.31 (s, 3H), 1.99 (s, 4H), 1.73 (p, J=6.6 Hz, 2H), 0.83 (s, 9H), −0.00 (s, 6H).

### 2-((3-(((3-Hydroxypropyl)amino)methyl)-4-(4-methylpiperazin-1-yl)phenyl)amino)-5-((triisopropylsilyl)ethynyl)pyrido[2,3-d]pyrimidin-7(8H)-one

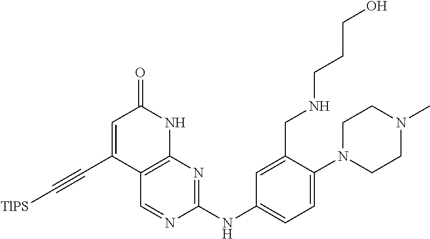

General procedure 13 was applied to 2-(methylsulfonyl)-5-((triisopropylsilyl)ethynyl)pyrido[2,3-d]pyrimidin-7(8H)-one (643 mg, 1.59 mmol), 3-(((3-((tert-butyldimethylsilyl)oxy)propyl)amino)methyl)-4-(4-methylpiperazin-1-yl)aniline (622 mg, 1.59 mmol) and trifluoroacetic acid (0.130 mL, 1.75 mmol) in acetonitrile (16 mL). The crude material was purified by flash column chromatography eluting with methanol (0-20%) in dichloromethane to yield the title compound as an orange solid (247 mg, 0.410 mmol, 37%).

(ES, m/z): [M+H]⁺=604.5

### 3⁴-(4-Methylpiperazin-1-yl)-1⁵-((triisopropylsilyl)ethynyl)-1⁷,1⁸-dihydro-2,5-diaza-1(2,8)-pyrido[2,3-d]pyrimidina-3(1,3)-benzenacyclooctaphan-1⁷-one

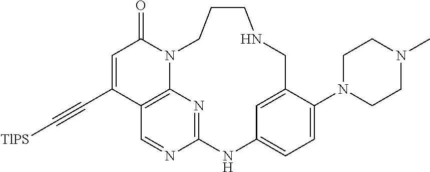

General procedure 20 was applied to 2-((3-(((3-hydroxypropyl)amino)methyl)-4-(4-methylpiperazin-1-yl)phenyl)amino)-5-((triisopropylsilyl)ethynyl)pyrido[2,3-d]pyrimidin-7(8H)-one (247 mg, 0.410 mmol), triphenylphosphine (430 mg, 1.64 mmol) and DIAD (0.300 mL, 1.64 mmol) in THF (20 mL). The crude material was purified by flash column chromatography eluting with methanol (0-20%) in dichloromethane to yield the title compound as an orange solid (127 mg, 0.217 mmol, 53%).

(ES, m/z): [M+H]⁺=586.5

### 1⁵-Ethynyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphan-1⁷-one

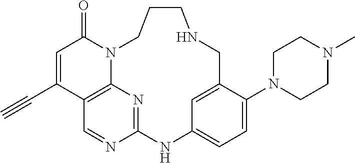 [SMILES: C#Cc1cc(=O)n2c3nc(ncc13)Nc1ccc(N3CCN(C)CC3)c(c1)CNCCC2]

General procedure 15 was applied to 3⁴-(4-methylpiperazin-1-yl)-1⁵-((triisopropylsilyl)ethynyl)-1⁷,1⁸-dihydro-2,5-diaza-1(2,8)-pyrido[2,3-d]pyrimidina-3(1,3)-benzenacyclooctaphan-1⁷-one (58.0 mg, 98.8 μmol) and potassium fluoride (115 mg, 1.98 mmol) in DMF (1.0 mL). The crude material was purified by flash column chromatography eluting with methanol (0-20%) in dichloromethane to yield the title compound as an orange solid (25.0 mg, 58.4 μmol, 59%).

(ES, m/z): [M+H]⁺=430.3

¹H NMR (500 MHz, DMSO) δ 8.49 (s, 1H), 6.95 (d, J=8.4 Hz, 1H), 6.46 (dd, J=8.4, 2.6 Hz, 1H), 6.40 (d, J=2.6 Hz, 1H), 6.29 (s, 1H), 4.97 (s, 2H), 4.84 (s, 2H), 4.78 (s, 1H), 4.30 (t, J=5.9 Hz, 2H), 3.43 (t, J=5.8 Hz, 2H), 2.72 (q, J=3.8 Hz, 4H), 2.44-2.27 (m, 4H), 2.16 (s, 3H).

### Example 200

### tert-Butyl((5-(2-fluoro-5-nitrophenoxy)pentyl)oxy)dimethylsilane

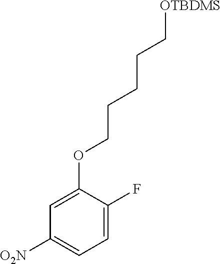 [SMILES: CC(C)(C)[Si](C)(C)OCCCCCOc1cc([N+](=O)[O-])ccc1F]

General procedure xx was applied to 2 fluoro-5-nitrophenol (1.20 g, 7.66 mmol), 5-((tert-butyldimethylsilyl)oxy)pentan-1-ol (1.84 g, 8.43 mmol), triphenylphosphine (2.61 g, 9.96 mmol) and DIAD (1.86 mL, 9.96 mmol) in THE (19 mL). The crude material was purified by flash column chromatography eluting with ethyl acetate (0-10%) in 40-60 petroleum ether to yield the title compound as an orange solid (3.26 g, 7.49 mmol, 83%).

### 1-(2-((5-((tert-Butyldimethylsilyl)oxy)pentyl)oxy)-4-nitrophenyl)-4-methylpiperazine

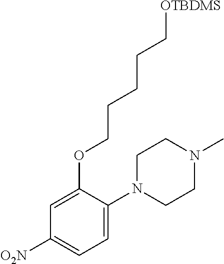 [SMILES: CN1CCN(c2ccc([N+](=O)[O-])cc2OCCCCCO[Si](C)(C)C(C)(C)C)CC1]

General procedure 11 was applied to tert-butyl((5-(2-fluoro-5-nitrophenoxy)pentyl)oxy)dimethylsilane (3.24 g, 9.06 mmol), lithium carbonate (803 mg, 10.9 mmol) and N-methylpiperazine (1.10 mL, 9.97 mmol) in acetonitrile (15 mL). The crude material was purified by flash column chromatography eluting with methanol (0-10%) in dichloromethane to yield the title compound as an orange solid (3.26 g, 7.49 mmol, 83%).

(ES, m/z): [M+H]⁺=438.4

¹H NMR (500 MHz, CDC₃) δ 7.79 (dd, J=8.8, 2.5 Hz, 1H), 7.53-7.45 (m, 1H), 6.81 (d, J=8.8 Hz, 1H), 4.01 (t, J=6.4 Hz, 2H), 3.60 (t, J=6.2 Hz, 2H), 3.22 (s, 4H), 2.54 (t, J=4.9 Hz, 4H), 2.30 (s, 3H), 1.89-1.79 (m, 4H), 1.61-1.44 (m, 4H), 0.84 (s, 9H), 0.00 (s, 6H).

### 3-((5-((tert-Butyldimethylsilyl)oxy)pentyl)oxy)-4-(4-methylpiperazin-1-yl)aniline

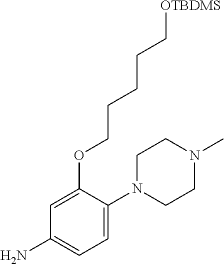 [SMILES: CN1CCN(c2ccc(N)cc2OCCCCCO[Si](C)(C)C(C)(C)C)CC1]

General procedure 12 was applied to 1-(2-((5-((tert-butyldimethylsilyl)oxy)pentyl)oxy)-4-nitrophenyl)-4-methylpiperazine (3.26 g, 7.49 mmol) and palladium on carbon (797 mg, 0.749 mmol) in ethanol (37 mL). The crude material was purified by flash column chromatography eluting with methanol (0-20%) in dichloromethane to yield the title compound as a purple oil (1.87 g, 4.61 mmol, 62%).

(ES, m/z): [M+H]⁺=408.3

¹H NMR (500 MHz, CDC₃) δ 6.67 (d, J=8.2 Hz, 1H), 6.19 (d, J=2.4 Hz, 1H), 6.16 (dd, J=8.3, 2.5 Hz, 1H), 3.85 (t, J=6.5 Hz, 2H), 3.58 (t, J=6.4 Hz, 2H), 3.00 (s, 4H), 2.65-2.61 (m, 4H), 2.35 (s, 3H), 1.78 (p, J=6.7 Hz, 2H), 1.59-1.49 (m, 2H), 1.48-1.38 (m, 2H), 0.84 (s, 9H), 0.00 (s, 6H).

### 2-((3-((5-Hydroxypentyl)oxy)-4-(4-methylpiperazin-1-yl)phenyl)amino)-5-((triisopropylsilyl)ethynyl)pyrido[2,3-d]pyrimidin-7(8H)-one

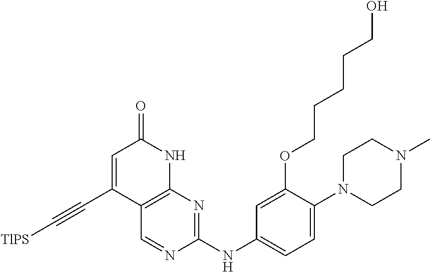

General procedure 13 was applied to 2-(methylsulfonyl)-5-((triisopropylsilyl)ethynyl)pyrido[2,3-d]pyrimidin-7(8H)-one (500 mg, 1.23 mmol), 3-((5-((tert-butyldimethylsilyl)oxy)pentyl)oxy)-4-(4-methylpiperazin-1-yl)aniline (500 mg, 1.23 mmol) and trifluoroacetic acid (0.100 mL, 1.36 mmol) in acetonitrile (12 mL). The crude material was purified by flash column chromatography eluting with methanol (0-20%) in dichloromethane to yield the title compound as an orange solid (678 mg, 1.10 mmol, 89%).

(ES, m/z): [M+H]⁺=619.5

### 3⁴-(4-Methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclononaphan-1⁷-one

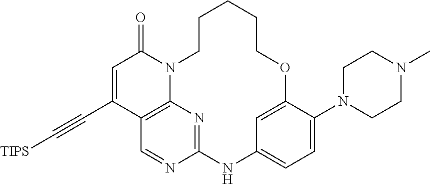

General procedure 20 was applied to 2-((3-((5-hydroxypentyl)oxy)-4-(4-methylpiperazin-1-yl)phenyl)amino)-5-((triisopropylsilyl)ethynyl)pyrido[2,3-d]pyrimidin-7(8H)-one (300 mg, 0.485 mmol), triphenylphosphine (509 mg, 1.94 mmol) and DIAD (0.360 mL, 1.94 mmol) in THF (20 mL). The crude material was purified by flash column chromatography eluting with methanol (0-20%) in dichloromethane to yield the title compound as an orange solid (65.2 mg, 0.108 mmol, 22%).

(ES, m/z): [M+H]⁺=601.5

¹H NMR (500 MHz, CDC₃) δ 8.79 (s, 1H), 8.05 (d, J=2.4 Hz, 1H), 7.39 (s, 1H), 6.85 (d, J=8.5 Hz, 1H), 6.55 (s, 1H), 6.45 (dd, J=8.5, 2.3 Hz, 1H), 4.27 (s, 2H), 4.20 (t, J=8.4 Hz, 2H), 3.04 (s, 4H), 2.60 (s, 4H), 2.32 (s, 3H), 1.98 (s, 2H), 1.82 (s, 2H), 1.60 (s, 2H), 1.18-1.09 (m, 3H), 1.08 (d, J=5.4 Hz, 18H).

### 1⁵-Ethynyl-34-(4-methylpiperazin-1-yl)-1⁷,1⁸-dihydro-4-oxa-2-aza-1(2,8)-pyrido[2,3-d]pyrimidina-3(1,3)-benzenacyclononaphan-17-one

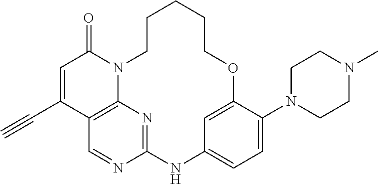 [SMILES: C#Cc1cc(=O)n2c3nc(ncc13)Nc1ccc(N3CCN(C)CC3)c(c1)OCCCCC2]

General procedure 15 was applied to 3⁴-(4-methylpiperazin-1-yl)-15-((triisopropylsilyl)ethynyl)-1⁷,1⁸-dihydro-4-oxa-2-aza-1(2,8)-pyrido[2,3-d]pyrimidina-3(1,3)-benzenacyclononaphan-17-one (65.0 mg, 0.108 mmol) and potassium fluoride (126 mg, 2.16 mmol) in DMF (1.0 mL). The crude material was purified by flash column chromatography eluting with methanol (0-20%) in dichloromethane to yield the title compound as an orange solid (33.2 mg, 77.4 μmol, 71%).

(ES, m/z): [M+H]⁺=445.3

¹H NMR (500 MHz, DMSO) δ 10.26 (s, 1H), 8.78 (s, 1H), 8.02 (d, J=2.3 Hz, 1H), 6.85 (d, J=8.6 Hz, 1H), 6.72 (dd, J=8.5, 2.2 Hz, 1H), 6.56 (s, 1H), 5.07 (s, 1H), 4.19 (d, J=9.6 Hz, 4H), 2.92 (s, 4H), 2.47 (s, 4H), 2.22 (s, 3H), 1.87 (s, 2H), 1.77 (s, 2H), 1.50 (s, 2H).

### Example 201

### 1-(2-Fluoro-5-nitrophenyl)methanamine

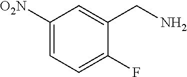 [SMILES: NCc1cc([N+](=O)[O-])ccc1F]

To a stirred solution of 2-fluoro-5-nitrobenzonitrile (20.0 g, 120 mmol, 1.0 eq.) in THF (400 mL) at room temperature was added borane-dimethyl sulfide complex (41.7 mL, 439 mmol, 3.6 eq.). The resulting mixture was stirred overnight at 80° C. The mixture was allowed to cool down to room temperature then acidified with 2 N aqueous hydrochloric acid (100 mL) dropwise at room temperature. The resulting mixture was stirred for 1 hour at 80° C. then concentrated under reduced pressure to yield the crude title compound as a yellow solid (32.7 g, crude).

### Methyl 3-[(tert-butyldiphenylsilyl)oxy]propanoate

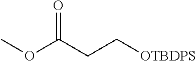

To a stirred solution of methyl 3-hydroxypropanoate (23.0 g, 221 mmol, 1.0 eq.) and DMF (230 mL) at 0° C. was added tert-butyldiphenylsilyl chloride (72.9 g, 265 mmol, 1.2 eq.), triethylamine (36.8 mL, 265 mmol, 1.2 eq.) and DMAP (2.70 g, 22.1 mmol, 0.1 eq.). The resulting mixture was stirred for 4-6 hours at room temperature then extracted with ethyl acetate (3×250 mL). The combined organic layers were washed with brine (2×200 mL), dried (Na₂SO₄) and concentrated under reduced pressure. The residue was purified by flash column chromatography eluting with 40-60 petroleum ether to afford the title compound as a colourless oil (34.5 g, 45%).

### 3-[(tert-Butyldiphenylsilyl)oxy]propanoic acid

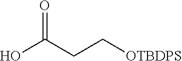

To a stirred solution of methyl 3-[(tert-butyldiphenylsilyl)oxy]propanoate (34.5 g, 101 mmol, 1.0 eq.) in ethanol (157 mL) at room temperature was added sodium hydroxide (4.23 g, 106 mmol, 1.05 eq.) and water (106 mL) dropwise over 1 minute. The resulting mixture was stirred for 4-6 hours at room temperature then neutralized to pH 7 with 1 N aqueous hydrochloric acid. The resulting mixture was extracted with ethyl acetate (3×50 mL). The combined organic layers were washed with brine (100 mL), dried (Na₂SO₄) and concentrated under reduced pressure to yield the crude title compound as a white solid (31.9 g, 96%).

### 3-[(tert-Butyldiphenylsilyl)oxy]-N-[(2-fluoro-5-nitrophenyl)methyl]propanamide

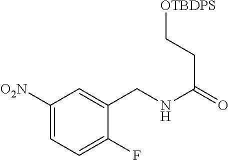

A solution of 3-[(tert-butyldiphenylsilyl)oxy]propanoic acid (20.0 g, 60.9 mmol, 1.0 eq.), 1-(2-fluoro-5-nitrophenyl)methanamine (12.4 g, 73.1 mmol, 1.2 eq.), EDCl (14.0 g, 73.1 mmol, 1.2 eq.), HOBT (9.87 g, 73.1 mmol, 1.2 eq.), triethylamine (25.4 mL, 183 mmol, 3.0 eq.) in dichloromethane (200 mL) was stirred overnight at room temperature. The resulting mixture was concentrated under reduced pressure. The residue was purified by flash column chromatography eluting with ethyl acetate (20% methanol) (10%) in 40-60 petroleum ether to afford the title compound as a brown yellow semi-solid (13.9 g, 47%).

### 3-[(tert-Butyldiphenylsilyl)oxy]-N-([2-(4-methylpiperazin-1-yl)-5-nitrophenyl]methylpropanamide

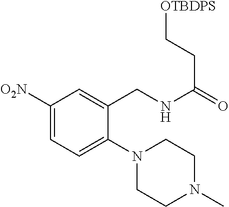

A solution of 3-[(tert-butyldiphenylsilyl)oxy]-N-[(2-fluoro-5-nitrophenyl)methyl]propanamide (5.00 g, 10.4 mmol, 1.0 eq.), N-methylpiperazine (1.25 g, 12.5 mmol, 1.2 eq.) and sodium carbonate (2.21 g, 20.8 mmol, 2.0 eq.) in acetonitrile (50 mL) was stirred overnight at 80° C. The resulting mixture was extracted with ethyl acetate (3×100 mL). The combined organic layers were washed with brine (150 mL), dried (Na₂SO₄) and concentrated under reduced pressure. The residue was purified by flash column chromatography eluting with ethyl acetate (20%) in 40-60 petroleum ether to afford the title compound as a brown solid (3.80 g, 65%).

### N-([5-Amino-2-(4-methylpiperazin-1-yl)phenyl]methyl-3-[(tert-butyldiphenylsilyl)oxy]propanamide

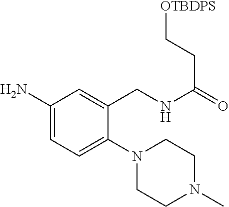

General procedure 12 was applied to 3-[(tert-butyldiphenylsilyl)oxy]-N-([2-(4-methylpiperazin-1-yl)-5-nitrophenyl]methylpropanamide (3.30 g, 5.88 mmol) and palladium on carbon (10%, 657 mg, 6.18 mmol) in THF (99 mL) to yield the crude title compound as a brown solid (2.88 g, 92%).

### 3-[(tert-Butyldiphenylsilyl)oxy]-N-([2-(4-methylpiperazin-1-yl)-5-((7-oxo-5-[2-(triisopropylsilyl)ethynyl]-8H-pyrido[2,3-d]pyrimidin-2-ylamino)phenyl]methylpropanamide

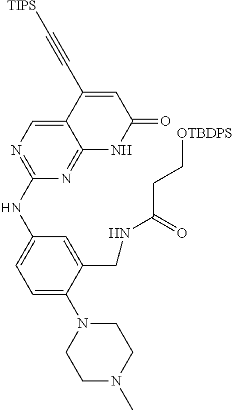

General procedure 13 was applied to N-([5-amino-2-(4-methylpiperazin-1-yl)phenyl]methyl-3-[(tert-butyldiphenylsilyl)oxy]propanamide (1.40 g, 2.4 mmol), 2-methanesulfonyl-5-[2-(triisopropylsilyl)ethynyl]-8H-pyrido[2,3-d]pyrimidin-7-one (1.28 g, 3.17 mmol) and trifluoroacetic acid (392 μL, 5.27 mmol) in 2-butanol (14 mL), stirred overnight at 80° C. The residue was purified by flash column chromatography eluting with methanol (8%) in dichloromethane to afford the title compound as a brown solid (1.83 g, 81%).

### 3-Hydroxy-N-([2-(4-methylpiperazin-1-yl)-5-((7-oxo-5-[2-(triisopropylsilyl)ethynyl]-8H-pyrido[2,3-d]pyrimidin-2-ylamino)phenyl]methylpropanamide

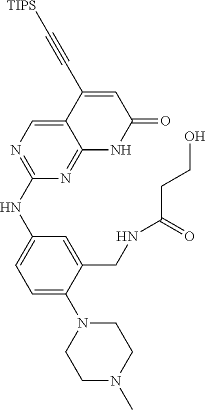

A solution of 3-[(tert-butyldiphenylsilyl)oxy]-N-([2-(4-methylpiperazin-1-yl)-5-((7-oxo-5-[2-(triisopropylsilyl)ethynyl]-8H-pyrido[2,3-d]pyrimidin-2-ylamino)phenyl]methylpropanamide (1.70 g, 1.98 mmol, 1.0 eq.) and hydrogen chloride in methanol (17 mL) was stirred for 4 hours at 60° C. The resulting mixture was concentrated under reduced pressure, to afford the crude title compound as a brown solid (718 mg, 58%).

### N-([2-(4-Methylpiperazin-1-yl)-5-((7-oxo-5-[2-(triisopropylsilyl)ethynyl]-8H-pyrido[2,3-d]pyrimidin-2-ylamino)phenyl]methylprop-2-enamide

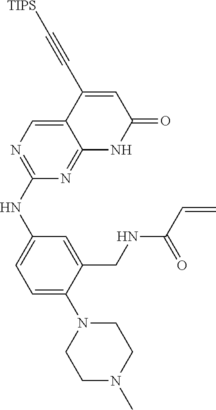

General procedure 20 was applied to 3-hydroxy-N-([2-(4-methylpiperazin-1-yl)-5-((7-oxo-5-[2-(triisopropylsilyl)ethynyl]-8H-pyrido[2,3-d]pyrimidin-2-ylamino)phenyl]methylpropanamide (150 mg, 0.243 mmol), triphenylphosphine (382 mg, 1.46 mmol) and DEAD (254 mg, 1.46 mmol) in DMF (1.0 mL). The residue was purified by reverse phase flash column chromatography eluting with acetonitrile (30-80%) in water (0.1% trifluoroacetic acid) to yield the title compound as a yellow solid (60.0 mg, 41%).

### N-([5-((5-Ethynyl-7-oxo-8H-pyrido[2,3-d]pyrimidin-2-ylamino)-2-(4-methylpiperazin-1-yl )phenyl]methylprop-2-enamide

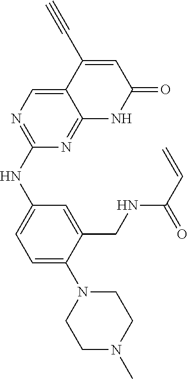 [SMILES: C#Cc1cc(=O)[nH]c2nc(Nc3ccc(N4CCN(C)CC4)c(CNC(=O)C=C)c3)ncc12]

General procedure 15 was applied to N-([2-(4-methylpiperazin-1-yl)-5-((7-oxo-5-[2-(triisopropylsilyl)ethynyl]-8H-pyrido[2,3-d]pyrimidin-2-ylamino)phenyl]methylprop-2-enamide (60.0 mg, 0.100 mmol) and potassium fluoride (58.1 mg, 1.00 mmol) in THF (0.50 mL), DMF (0.50 mL) and water (50 μL), stirred for 1 hour at room temperature. The crude product was purified by Prep-HPLC eluting with acetonitrile (9-25%) in water (0.1% formic acid), to afford the title compound as a yellow solid (9.00 mg, 20%).

(ES, m/z) [M+H]⁺=444.10

¹H-NMR (400 MHz, DMSO-d₆) b 12.07 (s, 1H), 10.06 (s, 1H), 8.70 (s, 1H), 8.45 (t, J=5.8 Hz, 1H), 7.82 (s, 1H), 7.71 (d, J=8.5 Hz, 1H), 7.07 (d, J=8.7 Hz, 1H), 6.44 (s, 1H), 6.34 (dd, J=17.1, 10.2 Hz, 1H), 6.15 (dd, J=17.1, 2.3 Hz, 1H), 5.62 (dd, J=10.2, 2.3 Hz, 1H), 5.02 (s, 1H), 4.44 (d, J=5.7 Hz, 2H), 2.83 (t, J=4.8 Hz, 4H), 2.46 (t, J=4.8 Hz, 4H), 2.24 (s, 3H).

### Example 202

### tert-Butyl 3-{[(4-methylbenzenesulfonyl)oxy]methyl}piperidine-1-carboxylate

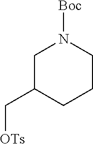 [SMILES: Cc1ccc(S(=O)(=O)OCC2CCCN(C(=O)OC(C)(C)C)C2)cc1]

To a stirred solution of tert-butyl 3-(hydroxymethyl)piperidine-1-carboxylate (6.00 g, 27.9 mmol, 1.0 eq.) in dichloromethane (30 mL) was added triethylamine (7.05 g, 69.7 mmol, 2.5 eq.) and tosyl chloride (6.38 g, 33.4 mmol, 1.2 eq.) in dichloromethane (30 mL) dropwise at 0° C. The resulting mixture was stirred for 4 hours at room temperature then quenched by the addition of saturated aqueous sodium hydrogen carbonate (100 mL). The resulting mixture was extracted with dichloromethane (3×50 mL). The combined organic layers were washed with brine (200 mL), dried (Na₂SO₄) and concentrated under reduced pressure. The residue was purified by flash column chromatography, eluting with ethyl acetate (15%) in 40-60 petroleum ether to afford the title compound as a colourless oil (9.50 g, 92%).

### tert-Butyl 3-[(methylsulfanyl)methyl]piperidine-1-carboxylate

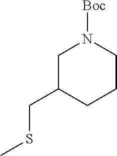 [SMILES: CSCC1CCCN(C(=O)OC(C)(C)C)C1]

To a stirred solution of tert-butyl 3-{[(4-methylbenzenesulfonyl)oxy]methyl}piperidine-1-carboxylate (4.70 g, 12.7 mmol, 1.0 eq.) in methanol (50 mL) was added sodiummethanethiolate (3.57 g, 50.9 mmol, 4.0 eq.). The resulting mixture was stirred overnight at 60° C. then poured into water and extracted with dichloromethane (3×50 mL). The combined organic layers were washed with brine (100 mL), dried (Na₂SO₄) then concentrated under reduced pressure to yield the title compound as a colourless oil (3.00 g, 96%). The crude product was used in the next step directly without further purification.

### tert-Butyl 3-(methanesulfonylmethyl)piperidine-1-carboxylate

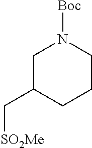 [SMILES: CC(C)(C)OC(=O)N1CCCC(CS(C)(=O)=O)C1]

To a stirred solution of tert-butyl 3-[(methylsulfanyl)methyl]piperidine-1-carboxylate (3.20 g, 13.0 mmol, 1.0 eq.) in methanol (15 mL) and water (15 mL) were added oxone (8.77 g, 52.2 mmol, 4.0 eq.) in portions at 0° C. The resulting mixture was stirred for 3 hours at room temperature then diluted with water (50 mL) and extracted with dichloromethane (3×50 mL). The combined organic layers were washed with brine (100 mL), dried (Na₂SO₄) and concentrated under reduced pressure to yield the title compound as a colourless oil (1.90 g, 52%). The crude product was used in the next step directly without further purification.

### 3-(Methanesulfonylmethyl)piperidine hydrochloride

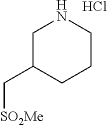 [SMILES: Cl ; CS(=O)(=O)CC1CCCNC1]

A solution of tert-butyl 3-(methanesulfonylmethyl)piperidine-1-carboxylate (1.90 g, 6.85 mmol, 1.0 eq.) and 4 M hydrochloric acid in dioxane (20 mL) was stirred for 2 hours at room temperature then concentrated under reduced pressure to yield the title compound as a white solid (1.44 g, 98%). The crude product was used in the next step directly without further purification.

### 3-(Methanesulfonylmethyl)-1-(2-nitrophenyl)piperidine

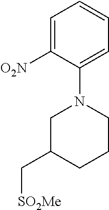 [SMILES: CS(=O)(=O)CC1CCCN(c2ccccc2[N+](=O)[O-])C1]

To a stirred solution of 3-(methanesulfonylmethyl)piperidine hydrochloride (1.44 g, 6.74 mmol, 1.0 eq.) and cesium carbonate (6.59 g, 20.2 mmol, 3.0 eq.) in acetonitrile (15 mL) was added O-fluoronitrobenzene (951 mg, 6.74 mmol, 1.0 eq.) at room temperature. The resulting mixture was stirred for 2 hours at 80° C. then diluted with water (30 mL) and extracted with dichloromethane (3×50 mL). The combined organic layers were washed with brine (100 mL), dried (Na₂SO₄) and concentrated under reduced pressure. The residue was purified by flash column chromatography eluting with ethyl acetate (15%) in 40-60 petroleum ether to afford the title compound as a white solid (1.73 g, 86%).

### 2-[3-(Methanesulfonylmethyl)piperidin-1-yl]aniline

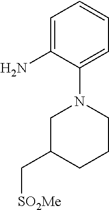 [SMILES: CS(=O)(=O)CC1CCCN(c2ccccc2N)C1]

General procedure 12 was applied to 3-(methanesulfonylmethyl)-1-(2-nitrophenyl)piperidine (1.73 g, 5.80 mmol) and palladium on carbon (20% wt, 350 mg) in methanol (20 mL) to yield the title compound as a white solid (1.36 g, 87%). The crude product was used in the next step directly without further purification.

### 8-Cyclopentyl-2-({2-[3-(methanesulfonylmethyl)piperidin-1-yl]phenyl}amino)-5-[2-(triisopropylsilyl)ethynyl]pyrido[2,3-d]pyrimidin-7-one

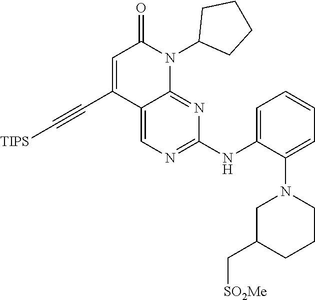

General procedure 13 was applied to 8-cyclopentyl-5-[2-(triisopropylsilyl)ethynyl]pyrido[2,3-d]pyrimidin-7-one (150 mg, 0.380 mmol), 2-[3-(methanesulfonylmethyl)piperidin-1-yl]aniline (153 mg, 0.570 mmol) and trifluoroacetic acid (86.5 mg, 0.760 mmol) in butan-2-ol (3.0 mL) stirred for 4 hours at 100° C. The residue was purified by flash column chromatography eluting with ethyl acetate (10%) in dichloromethane to afford the title compound as a yellow solid (160 mg, 64%).

### 8-Cyclopentyl-5-ethynyl-2-({2-[3-(methanesulfonylmethyl)piperidin-1-yl]phenyl}amino)pyrido[2,3-d]pyrimidin-7-one

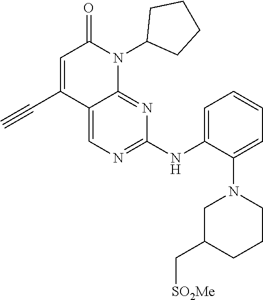 [SMILES: C#Cc1cc(=O)n(C2CCCC2)c2nc(Nc3ccccc3N3CCCC(CS(C)(=O)=O)C3)ncc12]

General procedure 15 was applied to 8-cyclopentyl-2-({2-[3-(methanesulfonylmethyl)piperidin-1-yl]phenyl}amino)-5-[2-(triisopropylsilyl)ethynyl]pyrido[2,3-d]pyrimidin-7-one (150 mg, 0.230 mmol) and potassium fluoride (132 mg, 2.30 mmol) in DMF (2.0 mL), THF (2.0 mL) and water (40 μL) stirred for 4 hours at 60° C. The residue was purified by reverse phase flash column chromatography eluting with acetonitrile (10-100%) in water (0.1% formic acid), to yield the title compound as a yellow solid (53.3 mg, 46%).

(ES, m/z) [M+H]⁺=506.20

¹H-NMR (300 MHz, DMSO-d₆) δ 9.18 (s, 1H), 8.76 (s, 1H), 8.39 (d, J=7.9 Hz, 1H), 7.24-7.15 (m, 2H), 7.12-7.03 (m, 1H), 6.93 (s, 1H), 5.68-5.57 (m, 1H), 5.05 (s, 1H), 3.21 (d, J=6.2 Hz, 2H), 3.18-3.09 (m, 1H), 2.99 (s, 3H), 2.93-2.81 (m, 1H), 2.76-2.57 (m, 2H), 2.46-2.30 (m, 1H), 2.08-1.87 (m, 3H), 1.85-1.56 (m, 8H), 1.44-1.28 (m, 1H).

### Example 203

### (1s,3s)-3-[(tert-Butoxycarbonyl)(methyl)amino]cyclobutane-1-carboxylic acid

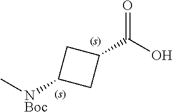

To a solution of (1s,3s)-3-[(tert-butoxycarbonyl)amino]cyclobutane-1-carboxylic acid (24.0 g, 111 mmol, 1.0 eq.) in THF was added sodium hydride (60% in oil, 8.00 g) at 0° C. The mixture was stirred for 15 minutes then methyl iodide (47.5 g, 334 mmol, 3.0 eq.) was added and the mixture warmed to room temperature and stirred for 16 hours. The reaction was quenched with water/ice at 0° C. then concentrated under reduced pressure. The resulting residue was washed with 2×200 mL of MTBE. The mixture was acidified to pH 4 with hydrochloric acid (2 M) then extracted with dichloromethane (2×300 mL). The combined organic layers were washed with brine (2×100 mL), dried (Na₂SO₄) and concentrated under reduced pressure to yield the title compound as a white solid (24.0 g, 94%).

### tert-Butyl N-methyl-N-[(1s,3s)-3-(hydroxymethyl)cyclobutyl]carbamate

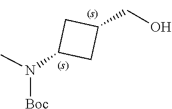

To a stirred solution of (1s,3s)-3-[(tert-butoxycarbonyl)(methyl)amino]cyclobutane-1-carboxylic acid (24.0 g, 105 mmol, 1.0 eq.) in THF (240 mL) was added borane-THF complex (20.0 mL, 209 mmol, 2.0 eq.) dropwise at 0° C. The resulting mixture was stirred overnight at room temperature under hydrogen atmosphere. The reaction was quenched with water/ice at 0° C. then extracted with ethyl acetate (2×300 mL). The combined organic layers were washed with brine (2×200 mL), dried (Na₂SO₄) and concentrated under reduced pressure to yield the title compound as a colourless oil (20.0 g, 89%).

### tert-Butyl N-methyl-N-[(1s,3s)-3-([(4-methylbenzenesulfonyl)oxy]methylcyclobutyl]carb amate

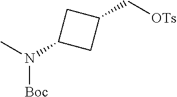 [SMILES: Cc1ccc(S(=O)(=O)OC[C@H]2C[C@@H](N(C)C(=O)OC(C)(C)C)C2)cc1]

To a stirred mixture of tert-butyl N-methyl-N-[(1s,3s)-3-(hydroxymethyl)cyclobutyl]carbamate (20.0 g, 93 mmol, 1.0 eq.) and triethylamine (32.3 mL, 232 mmol, 2.5 eq.) in dichloromethane (200 mL) was added tosyl chloride (19.5 g, 102 mmol, 1.1 eq.) in portions at room temperature. The resulting mixture was stirred overnight at room temperature. The residue was purified by reverse phase flash column chromatography eluting with acetonitrile (30-80%) in water (0.1% trifluoroacetic acid) to yield the title compound as a white solid (10.0 g, 29%).

### tert-Butyl N-methyl-N-[(1s,3s)-3-[(methylsulfanyl)methyl]cyclobutyl]carbamate

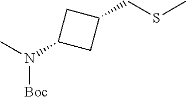

A mixture of tert-butyl N-methyl-N-[(1s,3s)-3-([(4-methylbenzenesulfonyl)oxy]methylcyclobutyl]carbamate (10.0 g, 27.0 mmol, 1.0 eq.) and sodiummethanethiolate (7.59 g, 108 mmol, 4.0 eq.) in DMF (100 mL) was stirred overnight at room temperature. The resulting mixture was extracted with ethyl acetate (2×200 mL). The combined organic layers were washed with brine (2×100 mL), dried (Na₂SO₄) and concentrated under reduced pressure to yield the title compound as a colourless solid (8.00 g, crude).

### tert-butyl N-methyl-N-[(1s,3s)-3-(methanesulfonylmethyl)cyclobutyl]carbamate

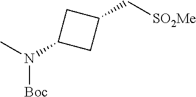 [SMILES: CN(C(=O)OC(C)(C)C)[C@H]1C[C@@H](CS(C)(=O)=O)C1]

A mixture of tert-butyl N-methyl-N-[(1s,3s)-3-[(methylsulfanyl)methyl]cyclobutyl]carbamate (2.00 g, 8.15 mmol, 1.0 eq.) and oxone (5.48 g, 32.6 mmol, 4.0 eq.) in methanol (10 mL) and water (10 mL) was stirred for 3 hours at room temperature. The resulting mixture was extracted with ethyl acetate (2×100 mL). The combined organic layers were washed with brine (2×100 mL), dried (Na₂SO₄) and concentrated under reduced pressure to yield the title compound as a colourless solid (1.50 g, 66.%).

### (1s,3s)-3-(Methanesulfonylmethyl)-N-methylcyclobutan-1-amine

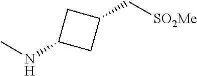

A solution of tert-butyl N-methyl-N-[(1s,3s)-3-(methanesulfonylmethyl)cyclobutyl]carbamate (1.50 g, 5.41 mmol, 1.0 eq.) in hydrogen chloride in 1,4-dioxane (15 mL) was stirred for 3 hours at room temperature then concentrated under reduced pressure to yield the title compound as a colourless solid (1.00 g, crude).

### N-Methyl-3-nitro-N-[(1s,3s)-3-(methanesulfonylmethyl)cyclobutyl]aniline

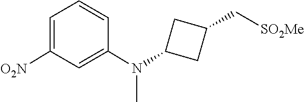 [SMILES: CN(c1cccc([N+](=O)[O-])c1)[C@H]1C[C@@H](CS(C)(=O)=O)C1]

To a solution of (1s,3s)-3-(methanesulfonylmethyl)-N-methylcyclobutan-1-amine (1.00 g, 5.64 mmol, 1.0 eq.) and 3-bromo-1-nitrobenzene (1.25 g, 6.21 mmol, 1.1 eq.) in dioxane (10 mL) was added cesium carbonate (3.68 g, 11.3 mmol, 2.0 eq.), RuPhos (260 mg, 0.564 mmol, 0.1 eq.) and Pd₂(dba)₃ (260 mg, 0.282 mmol, 0.05 eq.). After stirring overnight at 80° C., the reaction mixture was concentrated under reduced pressure. The residue was purified by flash column chromatography eluting with ethyl acetate (50%) in 40-60 petroleum ether to afford the title compound as a yellow solid (1.00 g, 59%).

### N¹-Methyl-N¹-[(1s,3s)-3-(methanesulfonylmethyl)cyclobutyl]benzene-1,3-diamine

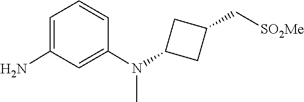 [SMILES: CN(c1cccc(N)c1)[C@H]1C[C@@H](CS(C)(=O)=O)C1]

General procedure 12 was applied to N-methyl-3-nitro-N-[(1s,3s)-3-(methanesulfonylmethyl)cyclobutyl]aniline (700 mg, 2.35 mmol) and palladium on carbon (140 mg, 1.32 mmol) in methanol (20 mL) to yield the title compound as a yellow solid (600 mg, 95%).

### 8-Cyclopentyl-2-[(3-(methyl[(1s,3s)-3-(methanesulfonylmethyl)cyclobutyl]aminophenyl)amino]-5-[2-(triisopropylsilyl)ethynyl]pyrido[2,3-d]pyrimidin-7-one

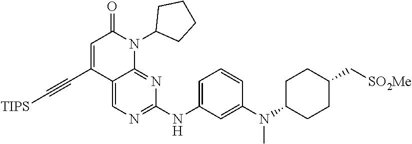

General procedure 13 was applied to 8-cyclopentyl-2-methanesulfonyl-5-[2-(triisopropylsilyl)ethynyl]pyrido[2,3-d]pyrimidin-7-one (150 mg, 0.317 mmol), trifluoroacetic acid (72.2 mg, 0.634 mmol) and N¹-methyl-N¹-[(1s,3s)-3-(methanesulfonylmethyl)cyclobutyl]benzene-1,3-diamine (102 mg, 0.380 mmol) in butan-2-ol (2.0 mL), stirred for 1 hour at 100° C. The residue was purified by reverse phase flash column chromatography eluting with acetonitrile (50-100%) in water (0.1% trifluoroacetic acid), to yield the title compound as a yellow solid (100 mg, 48%).

### 8-Cyclopentyl-5-ethynyl-2-[(3-(methyl[(1s,3s)-3-(methanesulfonylmethyl)cyclobutyl]aminophenyl)amino]pyrido[2,3-d]pyrimidin-7-one

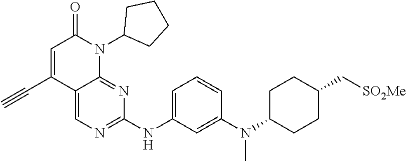 [SMILES: C#Cc1cc(=O)n(C2CCCC2)c2nc(Nc3cccc(N(C)[C@H]4CC[C@@H](CS(C)(=O)=O)CC4)c3)ncc12]

General procedure 15 was applied to 8-cyclopentyl-2-[(3-(methyl[(1s,3s)-3-(methanesulfonylmethyl)cyclobutyl]aminophenyl)amino]-5-[2-(triisopropylsilyl)ethynyl]pyrido[2,3-d]pyrimidin-7-one (80.0 mg, 0.121 mmol) and potassium fluoride (70.2 mg, 1.21 mmol) in THF (0.5 mL), DMF (0.5 mL) and water (80 μL), stirred for 2 hours at room temperature. The crude product was purified by Prep-HPLC eluting with acetonitrile (35-58%) in water (0.1% formic acid), to yield the title compound as a yellow solid (26.0 mg, 41%).

(ES, m/z) [M+H]⁺=506.20

¹H-NMR (400 MHz, DMSO-d₆) δ 9.99 (s, 1H), 9.15 (s, 1H), 7.67 (s, 1H), 7.21-7.03 (m, 2H), 6.89 (s, 1H), 6.47 (dt, J=7.5, 2.0 Hz, 1H), 5.59 (dq, J=6.4, 3.2 Hz, 1H), 5.05 (s, 1H), 3.97-3.79 (m, 1H), 3.29 (d, J=7.2 Hz, 2H), 2.93 (s, 3H), 2.80 (s, 3H), 2.72-2.60 (m, 2H), 2.42 (dt, J=9.4, 7.4 Hz, 1H), 2.11-1.87 (m, 4H), 1.82-1.56 (m, 6H).

### Example 204

### (1s,4s)-4-[(tert-Butoxycarbonyl)(methyl)amino]cyclohexane-1-carboxylic acid

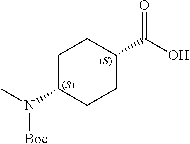

To a stirred solution of (1s,4s)-4-[(tert-butoxycarbonyl)amino]cyclohexane-1-carboxylic acid (23.5 g, 96.6 mmol, 1.0 eq.) in THF was added sodium hydride (11.6 g, 483 mmol, 5.0 eq.) at 0° C. The reaction mixture was stirred for 30 minutes at 0° C. then methyl iodide (18.0 mL, 290 mmol, 3.0 eq.) was added at 0° C. The reaction was stirred for overnight at room temperature then quenched with water/ice (500 mL) and concentrated under reduced pressure. The aqueous layer was extracted with MTBE (200 mL). The aqueous layer was acidified to pH 3 with aqueous hydrochloric acid then extracted with dichloromethane (3×250 mL). The combined organic layers were washed brine (200 mL), dried (Na₂SO₄) and concentrated under reduced pressure to yield the title compound as a white solid (23.4 g, 94%).

### tert-Butyl N-methyl-N-[(1s,4s)-4-(hydroxymethyl)cyclohexyl]carbamate

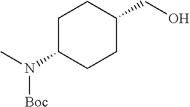 [SMILES: CN(C(=O)OC(C)(C)C)[C@H]1CC[C@@H](CO)CC1]

To a stirred solution of (1s,4s)-4-[(tert-butoxycarbonyl)(methyl)amino]cyclohexane-1-carboxylic acid (23.0 g, 89.4 mmol, 1.0 eq.) in THF (50 mL) was added borane-THF complex (1 M, 207 mL, 2.0 eq.) in portions at 0° C. The resulting mixture was stirred for 2 hours at room temperature then quenched with water/ice (300 mL). The resulting mixture was concentrated under reduced pressure then extracted with dichloromethane (2×300 mL). The combined organic layers were washed with brine (2×100 mL), dried (Na₂SO₄) and concentrated under reduced pressure to yield the title compound as a colourless oil (20.0 g, 92%).

### tert-Butyl N-methyl-N-[(1s,4s)-4-([(4-methylbenzenesulfonyl)oxy]methylcyclohexyl]carbamate

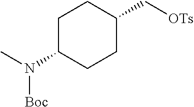 [SMILES: Cc1ccc(S(=O)(=O)OC[C@H]2CC[C@@H](N(C)C(=O)OC(C)(C)C)CC2)cc1]

A solution of tert-butyl N-methyl-N-[(1s,4s)-4-(hydroxymethyl)cyclohexyl]carbamate (20.0 g, 82.2 mmol, 1.0 eq.), tosyl chloride (23.5 g, 123 mmol, 1.5 eq.) and triethylamine (34.3 mL, 246 mmol, 3.0 eq.) in dichloromethane (200 mL) was stirred for 4 hours at room temperature. The residue was purified by reverse phase flash column chromatography eluting with acetonitrile (50-68%) in water (0.1% formic acid), to afford the title compound as a white solid (19.0 g, 58%).

### tert-Butyl N-methyl-N-[(1s,4s)-4-[(methylsulfanyl)methyl]cyclohexyl]carbamate

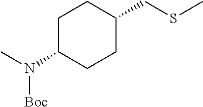

A solution of tert-butyl N-methyl-N-[(1s,4s)-4-([(4-methylbenzenesulfonyl)oxy]methylcyclohexyl]carbamate (10.0 g, 25.2 mmol, 1.0 eq.) and (methylsulfanyl)sodium (5.29 g, 75.5 mmol, 3.0 eq.) in methanol (100 mL) was stirred for overnight at room temperature. The resulting mixture was washed with saturated aqueous sodium hydrogen carbonate (300 mL) and extracted with ethyl acetate (3×150 mL). The combined organic layers were washed with brine (200 mL), dried (Na₂SO₄) and concentrated under reduced pressure to afford the crude title compound as a colourless oil (6.86 g, 100%).

### tert-Butyl N-methyl-N-[(1s,4s)-4-methanesulfonylmethyl)cyclohexyl]carbamate

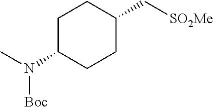 [SMILES: CN(C(=O)OC(C)(C)C)[C@H]1CC[C@@H](CS(C)(=O)=O)CC1]

To a stirred solution of tert-butyl N-methyl-N-[(1s,4s)-4-[(methylsulfanyl)methyl]cyclohexyl]carbamate (5.00 g, 18.3 mmol, 1.0 eq.) in THF (50.0 mL) and water (50.0 mL) was added oxone (15.4 g, 91.4 mmol, 5.0 eq.) at 0° C. The reaction mixture was stirred for 3 hours at room temperature then extracted with ethyl acetate (3×100 mL). The combined organic layers were washed with brine (150 mL), dried (Na₂SO₄) and concentrated under reduced pressure to yield the crude title compound as a colourless oil (4.92 g, 88%).

### (1s,4s)-4-(Methanesulfonylmethyl)-N-methylcyclohexan-1-amine

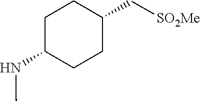

A solution of tert-butyl N-methyl-N-[(1s,4s)-4-(methanesulfonylmethyl)cyclohexyl]carbamate (5.00 g, 16.4 mmol, 1.0 eq.) and hydrogen chloride in methanol (50.0 mL, 1645 mmol, 100 eq.) was stirred for 2 hours at room temperature. The resulting mixture was concentrated under reduced pressure to afford the crude title compound as an off-white solid (3.75 g, 111%).

### N-Methyl-3-nitro-N-[(1s,4s)-4-(methanesulfonylmethyl)cyclohexyl]aniline

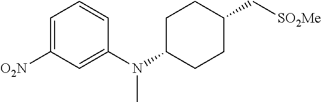 [SMILES: CN(c1cccc([N+](=O)[O-])c1)[C@H]1CC[C@@H](CS(C)(=O)=O)CC1]

A solution of (1s,4s)-4-(methanesulfonylmethyl)-N-methylcyclohexan-1-amine (2.00 g, 9.74 mmol, 1.0 eq.), 3-bromo-1-nitrobenzene (2.16 g, 10.7 mmol, 1.1 eq.), cesium carbonate (6.35 g, 19.5 mmol, 2.0 eq.), Ruphos (455 mg, 0.974 mmol, 0.1 eq.) and Pd₂(dba)₃ (446 mg, 0.487 mmol, 0.05 eq.) in dioxane (22 mL) was stirred overnight at 80° C. The resulting mixture was concentrated under reduced pressure. The residue was purified by flash column chromatography eluting with ethyl acetate (50%) in 40-60 petroleum ether to afford the title compound as a reddish brown solid (1.57 g, 49%).

### N¹-Methyl-N¹-[(1s,4s)-4-(methanesulfonylmethyl)cyclohexyl]benzene-1,3-diamine

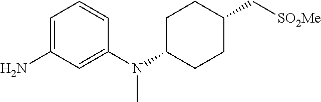 [SMILES: CN(c1cccc(N)c1)[C@H]1CC[C@@H](CS(C)(=O)=O)CC1]

General procedure 12 was applied to N-methyl-3-nitro-N-[(1s,4s)-4-(methanesulfonylmethyl)cyclohexyl]aniline (200 mg, 0.613 mmol) and palladium on carbon (10%, 70 mg, 0.658 mmol) in THF (20 mL) to yield the crude title compound as a brown oil (232 mg, 127%).

### 8-Cyclopentyl-2-[(3-(methyl[(1s,4s)-4-(methanesulfonylmethyl)cyclohexyl]aminophenyl)amino]-5-[2-(triisopropylsilyl)ethynyl]pyrido[2,3-d]pyrimidin-7-one

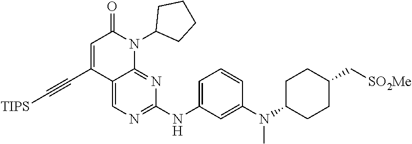

General procedure 13 was applied to 8-cyclopentyl-2-methanesulfonyl-5-[2-(triisopropylsilyl)ethynyl]pyrido[2,3-d]pyrimidin-7-one (140 mg, 0.296 mmol), N¹-methyl-N¹-[(1s,4s)-4-(methanesulfonylmethyl)cyclohexyl]benzene-1,3-diamine (131 mg, 0.444 mmol) and trifluoroacetic acid (45.3 uL, 0.592 mmol) in 2-butanol (1.4 mL) stirred for 2 hours at 100° C. The residue was purified by reverse phase flash column chromatography eluting with acetonitrile (10-100%) in water (0.1% formic acid), to afford the title compound as a brown yellow solid (76.1 mg, 37%).

### 8-Cyclopentyl-5-ethynyl-2-[(3-(methyl[(1s,4s)-4-(methanesulfonylmethyl)cyclohexyl]aminophenyl)amino]pyrido[2,3-d]pyrimidin-7-one

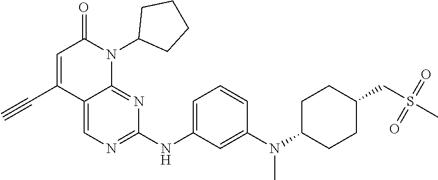 [SMILES: C#Cc1cc(=O)n(C2CCCC2)c2nc(Nc3cccc(N(C)[C@H]4CC[C@@H](CS(C)(=O)=O)CC4)c3)ncc12]

General procedure 15 was applied to 8-cyclopentyl-2-[(3-(methyl[(1s,4s)-4-(methanesulfonylmethyl)cyclohexyl]aminophenyl)amino]-5-[2-(triisopropylsilyl)ethynyl]pyrido[2,3-d]pyrimidin-7-one (65.0 mg, 94.0 μmol) and potassium fluoride (54.7 mg, 0.940 mmol) in THF (1.0 mL), DMF (1.0 mL) and water (65 μL) stirred for 2 hours at 60° C. The residue was purified by reverse phase flash column chromatography eluting with acetonitrile (10-47%) in water (0.1% formic acid), to yield the title compound as a yellow solid (22.9 mg, 44%).

(ES, m/z) [M+H]⁺=534.30

¹H-NMR (400 MHz, DMSO-d₆) δ 9.91 (d, J=4.4 Hz, 1H), 9.14 (s, 1H), 7.66 (s, 1H), 7.12-7.07 (m, 2H), 6.89 (s, 1H), 6.50-6.37 (m, 1H), 5.61 (d, J=19.9 Hz, 1H), 5.04 (d, J=1.4 Hz, 1H), 3.62 (s, 1H), 3.35 (s, 2H), 2.99 (d, J=6.2 Hz, 3H), 2.77 (d, J=18.4 Hz, 3H), 2.38 (s, 1H), 2.10-1.44 (m, 16H).

### Example 205

### (1r,3r)-3-[(tert-butoxycarbonyl)(methyl)amino]cyclobutane-1-carboxylic acid

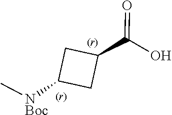 [SMILES: CN(C(=O)OC(C)(C)C)[C@H]1C[C@H](C(=O)O)C1]

To a solution of (1r,3r)-3-[(tert-butoxycarbonyl)amino]cyclobutane-1-carboxylic acid (10.0 g, 46.4 mmol, 1.0 eq.) in THF was added sodium hydride (60% in oil, 3.34 g) at 0° C. The mixture was stirred for 15 minutes then methyl iodide (19.8 g, 139 mmol, 3.0 eq.) was added and the mixture warmed to room temperature and stirred for 16 hours. The reaction was quenched with water/ice at 0° C. then concentrated under reduced pressure. The resulting residue was washed with MTBE (2×200 mL) then acidified to pH 4 with aqueous hydrochloric acid (2 M). The resulting mixture was extracted with dichloromethane (2×300 mL). The combined organic layers were washed with brine (2×100 mL), dried (Na₂SO₄) and concentrated under reduced pressure to yield the title compound as a white solid (8.00 g, 75%).

### tert-butyl N-methyl-N-[(1r,3r)-3-(hydroxymethyl)cyclobutyl]carbamate

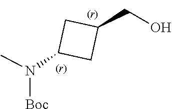

To a stirred solution of (1r,3r)-3-[(tert-butoxycarbonyl)(methyl)amino]cyclobutane-1-carboxylic acid (8.00 g, 34.9 mmol, 1.0 eq.) in THF (240 mL) was added borane-THF complex (6.68 mL, 69.8 mmol, 2.0 eq.) dropwise at 0° C. The resulting mixture was stirred overnight at room temperature under a hydrogen atmosphere. The reaction was quenched with water/ice then extracted with ethyl acetate (2×300 mL). The combined organic layers were washed with brine (2×200 mL), dried (Na₂SO₄) and concentrated under reduced pressure to yield the title compound as a colourless oil (8.00 g, crude).

### tert-Butyl N-methyl-N-[(1r,3r)-3-([(4-methylbenzenesulfonyl)oxy]methylcyclobutyl]carb amate

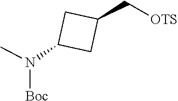

To a stirred mixture of tert-butyl N-methyl-N-[(1r,3r)-3-(hydroxymethyl)cyclobutyl]carbamate (8.00 g, 37.2 mmol, 1.0 eq.) and triethylamine (12.9 mL, 92.9 mmol, 2.5 eq.) in dichloromethane (80 mL) was added tosyl chloride (7.79 g, 40.9 mmol, 1.1 eq.) in portions at room temperature. The resulting mixture was stirred overnight at room temperature. The residue was purified by reverse phase flash column chromatography eluting acetonitrile (30-80%) in water (0.1% trifluoroacetic acid), to yield the title compound as a white solid (4.00 g, 29%).

### tert-Butyl N-methyl-N-[(1r,3r)-3-[(methylsulfanyl)methyl]cyclobutyl]carbamate

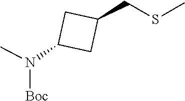

A mixture of tert-butyl N-methyl-N-[(1r,3r)-3-([(4-methylbenzenesulfonyl)oxy]methylcyclobutyl]carbamate (4.00 g, 10.8 mmol, 1.0 eq.) and sodium ethanethiolate (3.03 g, 43.3 mmol, 4.0 eq.) in methanol (40 mL) was stirred overnight at room temperature. The resulting mixture was extracted with ethyl acetate (2×200 mL). The combined organic layers were washed with brine (2×100 mL), dried (Na₂SO₄) and concentrated under reduced pressure to yield the title compound as a colourless solid (2.00 g, 75%).

### tert-Butyl N-methyl-N-[(1r,3r)-3-(methanesulfonylmethyl)cyclobutyl]carbamate

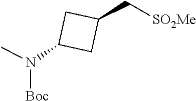 [SMILES: CN(C(=O)OC(C)(C)C)[C@H]1C[C@H](CS(C)(=O)=O)C1]

A mixture of tert-butyl N-methyl-N-[(1r,3r)-3-[(methylsulfanyl)methyl]cyclobutyl]carbamate (2.00 g, 8.15 mmol, 1.0 eq.) and oxone (5.48 g, 32.6 mmol, 4.0 eq.) in methanol (10 mL) and water (10 mL) were stirred for 3 hours at room temperature. The resulting mixture was extracted with ethyl acetate (2×100 mL). The combined organic layers were washed with brine (2×100 mL), dried (Na₂SO₄) and concentrated under reduced pressure to yield the title compound as a colourless solid (1.20 g, 53%).

### (1r,3r)-3-(Methanesulfonylmethyl)-N-methylcyclobutan-1-amine

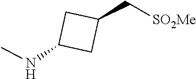

A solution of tert-butyl N-methyl-N-[(1r,3r)-3-(methanesulfonylmethyl)cyclobutyl]carbamate (1.20 g, 4.33 mmol, 1.0 eq.) in hydrogen chloride in 1,4-dioxane (12 mL) was stirred for 3 hours at room temperature then concentrated under reduced pressure to yield the title compound as a colourless solid (800 mg, crude).

### N-Methyl-3-nitro-N-[(1r,3r)-3-(methanesulfonylmethyl)cyclobutyl]aniline

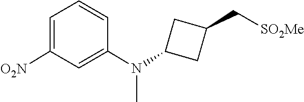 [SMILES: CN(c1cccc([N+](=O)[O-])c1)[C@H]1C[C@H](CS(C)(=O)=O)C1]

To a solution of (1r,3r)-3-(methanesulfonylmethyl)-N-methylcyclobutan-1-amine (800 mg, 4.51 mmol, 1.0 eq.) and 3-bromo-1-nitrobenzene (1.09 g, 5.42 mmol, 1.2 eq.) in dioxane (10 mL) was added cesium carbonate (2.94 g, 9.026 mmol, 2.0 eq.), RuPhos (210 mg, 0.451 mmol, 0.1 eq.) and Pd₂(dba)₃ (206 mg, 0.226 mmol, 0.05 eq.). After stirring overnight at 80° C., the reaction mixture was concentrated under reduced pressure. The residue was purified by flash column chromatography eluting with ethyl acetate (50%) in 40-60 petroleum ether to afford the title compound as a yellow solid (760 mg, 56%).

### N¹-Methyl-N¹-[(1r,3r)-3-(methanesulfonylmethyl)cyclobutyl]benzene-1,3-diamine

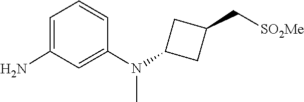 [SMILES: CN(c1cccc(N)c1)[C@H]1C[C@H](CS(C)(=O)=O)C1]

General procedure 12 was applied to N-methyl-3-nitro-N-[(1r,3r)-3-(methanesulfonylmethyl)cyclobutyl]aniline (700 mg, 2.35 mmol) and palladium on carbon (140 mg) in methanol (7 mL) to yield the title compound as a yellow solid (500 mg, 79%).

### 8-Cyclopentyl-2-[(3-(methyl[(1r,3r)-3-(methanesulfonylmethyl)cyclobutyl]aminophenyl) amino]-5-[2-(triisopropylsilyl)ethynyl]pyrido[2,3-d]pyrimidin-7-one

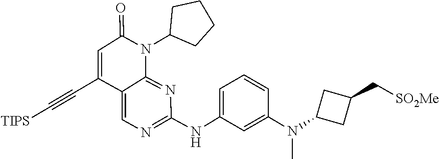

General procedure 13 was applied to 8-cyclopentyl-2-methanesulfonyl-5-[2-(triisopropylsilyl)ethynyl]pyrido[2,3-d]pyrimidin-7-one (150 mg, 0.317 mmol), trifluoroacetic acid (72.2 mg, 0.634 mmol) and N¹-methyl-N¹-[(1r,3r)-3-(methanesulfonylmethyl)cyclobutyl]benzene-1,3-diamine (102 mg, 0.380 mmol) in butan-2-ol (2.0 mL), stirred for 1 hour at 100° C. The residue was purified by reverse phase flash column chromatography eluting with acetonitrile (50-100%) in water (0.1% trifluoroacetic acid), to yield the title compound as a yellow solid (80.0 mg, 38%).

### 8-Cyclopentyl-5-ethynyl-2-[(3-(methyl[(1r,3r)-3-(methanesulfonylmethyl)cyclobutyl]aminophenyl)amino]pyrido[2,3-d]pyrimidin-7-one

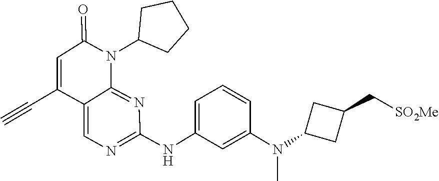 [SMILES: C#Cc1cc(=O)n(C2CCCC2)c2nc(Nc3cccc(N(C)[C@H]4C[C@H](CS(C)(=O)=O)C4)c3)ncc12]

General procedure 15 was applied to 8-cyclopentyl-2-[(3-(methyl[(1r,3r)-3-(methanesulfonylmethyl)cyclobutyl]aminophenyl)amino]-5-[2-(triisopropylsilyl)ethynyl]pyrido[2,3-d]pyrimidin-7-one (70.0 mg, 0.106 mmol) and potassium fluoride (61.4 mg, 1.06 mmol) in THF (0.5 mL), DMF (0.5 mL) and water (50 μL), stirred for 2 hours at room temperature. The crude product was purified by Prep-HPLC eluting with acetonitrile (50-80%) in water (0.1% formic acid to afford the title compound as a yellow solid (24.0 mg, 43%).

(ES, m/z) [M+H]⁺=506.35

¹H-NMR (400 MHz, DMSO-d₆) δ 9.94 (s, 1H), 9.16 (s, 1H), 7.42 (s, 1H), 7.26 (d, J=8.0 Hz, 1H), 7.13 (t, J=8.1 Hz, 1H), 6.88 (s, 1H), 6.49 (dd, J=8.3, 2.3 Hz, 1H), 5.58 (tt, J=6.4, 2.8 Hz, 1H), 5.03 (s, 1H), 4.18 (p, J=7.5 Hz, 1H), 3.43 (d, J=7.4 Hz, 2H), 2.95 (s, 3H), 2.81 (s, 3H), 2.64 (d, J=7.8 Hz, 1H), 2.43-2.25 (m, 4H), 2.08-1.95 (m, 2H), 1.83-1.58 (m, 6H).

### Example 206

### (1r,4r)-4-[(tert-Butoxycarbonyl)(methyl)amino]cyclohexane-1-carboxylic acid

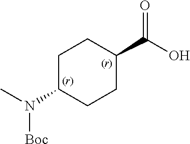

To a stirred solution of (1r,4r)-4-[(tert-butoxycarbonyl)amino]cyclohexane-1-carboxylic acid (20.0 g, 82.2 mmol, 1.0 eq.) in THF (200 mL) was added sodium hydride (9.86 g, 411 mmol, 5.0 eq.) at 0° C. The resulting mixture was stirred for 30 minutes at 0° C. then methyl iodide (15.3 mL, 247 mmol, 3.0 eq.) was added at 0° C. The reaction mixture was stirred overnight at room temperature then quenched with water/ice (200 mL). The resulting mixture was concentrated under reduced pressure then extracted with MTBE (100 mL). The mixture was acidified to pH 3 with aqueous hydrochloric acid and extracted with dichloromethane (3×100 mL). The combined organic layers were washed with brine (150 mL), dried (Na₂SO₄) and concentrated under reduced pressure to yield the crude title compound as a white solid (20.3 g, 96%).

### tert-Butyl N-methyl-N-[(1r,4r)-4-(hydroxymethyl)cyclohexyl]carbamate

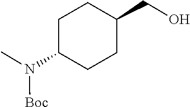 [SMILES: CN(C(=O)OC(C)(C)C)[C@H]1CC[C@H](CO)CC1]

To a stirred solution of (1r,4r)-4-[(tert-butoxycarbonyl)(methyl)amino]cyclohexane-1-carboxylic acid (20.3 g, 78.9 mmol, 1.0 eq.) in THF (500 mL) at 0° C. was added borane-THF complex (13.6 g, 158 mmol, 2.0 eq.). The resulting mixture was stirred overnight at room temperature then quenched with water/ice (400 mL). The resulting mixture was concentrated under reduced pressure then extracted with dichloromethane (3×300 mL). The combined organic layers were washed with brine (500 mL), dried (Na₂SO₄) and concentrated under reduced pressure to afford the crude title compound as a white solid (18.9 g, 98%).

### tert-Butyl N-methyl-N-[(1r,4r)-4-([(4-methylbenzenesulfonyl)oxy]methylcyclohexyl]carb amate

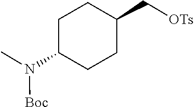 [SMILES: Cc1ccc(S(=O)(=O)OC[C@H]2CC[C@H](N(C)C(=O)OC(C)(C)C)CC2)cc1]

A solution of tert-butyl N-methyl-N-[(1r,4r)-4-(hydroxymethyl)cyclohexyl]carbamate (18.0 g, 74.0 mmol, 1.0 eq.), tosyl chloride (21.1 g, 111 mmol, 1.5 eq.) and triethylamine (30.8 mL, 222 mmol, 3.0 eq.) in dichloromethane (180 mL) was stirred for 4 hours at room temperature. The resulting mixture was filtered washing with dichloromethane (3×20 mL). The filtrate was concentrated under reduced pressure. The residue was purified by reverse phase flash column chromatography eluting with acetonitrile (50-68%) in water (0.1% formic acid), to afford the title compound as a white solid (23.0 g, 78%).

### tert-Butyl N-methyl-N-[(1r,4r)-4-[(methylsulfanyl)methyl]cyclohexyl]carbamate

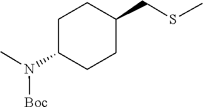

A solution of tert-butyl N-methyl-N-[(1r,4r)-4-([(4-methylbenzenesulfonyl)oxy]methylcyclohexyl]carbamate (12.0 g, 30.2 mmol, 1.0 eq.) and (methylsulfanyl)sodium (8.46 g, 121 mmol, 4.0 eq.) in methanol (120 mL) was stirred for 2 hours at room temperature. The resulting mixture was extracted with ethyl acetate (3×150 mL). The combined organic layers were washed with brine (150 mL), dried (Na₂SO₄) and concentrated under reduced pressure to yield the crude title compound as a white semi-solid (7.58 g, 92%).

### tert-Butyl N-methyl-N-[(1r,4r)-4-(methanesulfonylmethyl)cyclohexyl]carbamate

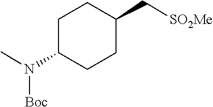 [SMILES: CN(C(=O)OC(C)(C)C)[C@H]1CC[C@H](CS(C)(=O)=O)CC1]

To a stirred solution of tert-butyl N-methyl-N-[(1r,4r)-4-[(methylsulfanyl)methyl]cyclohexyl]carbamate (7.00 g, 25.6 mmol, 1.0 eq.) in THF (70 mL) and water (70 mL) at 0° C. was added oxone (25.8 g, 154 mmol, 6.0 eq.). The resulting mixture was stirred for 2 hours at room temperature then extracted with ethyl acetate (3×100 mL). The combined organic layers were washed with brine (100 mL), dried (Na₂SO₄) and concentrated under reduced pressure to yield the crude title compound as a colourless oil (7.15 g, 91%).

### (1 r,4r)-4-(Methanesulfonylmethyl)-N-methylcyclohexan-1-amine

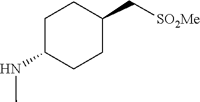

A solution of tert-butyl N-methyl-N-[(1r,4r)-4-(methanesulfonylmethyl)cyclohexyl]carbamate (2.00 g, 6.55 mmol, 1.0 eq.) and hydrogen chloride in methanol (20 mL) was stirred for 2 hours at room temperature then concentrated under reduced pressure, to afford the crude title compound as a white solid (1.59 g, 119%).

### N-Methyl-3-nitro-N-[(1r,4r)-4-(methanesulfonylmethyl)cyclohexyl]aniline

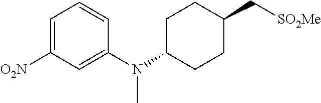 [SMILES: CN(c1cccc([N+](=O)[O-])c1)[C@H]1CC[C@H](CS(C)(=O)=O)CC1]

A solution of (1r,4r)-4-(methanesulfonylmethyl)-N-methylcyclohexan-1-amine (1.60 g, 7.79 mmol, 1.0 eq.), 3-bromo-1-nitrobenzene (1.73 g, 8.57 mmol, 1.1 eq.), cesium carbonate (5.08 g, 15.6 mmol, 2.0 eq.), Ruphos (364 mg, 0.779 mmol, 0.1 eq.) and Pd₂(dba)₃ (357 mg, 0.390 mmol, 0.05 eq.) in dioxane (16 mL) was stirred overnight at 80° C. The resulting mixture was concentrated under reduced pressure. The residue was purified by flash column chromatography eluting with ethyl acetate (40%) in 40-60 petroleum ether to afford the title compound as a reddish brown solid (1.74 g, 68%).

### N¹-Methyl-N¹-[(1r,4r)-4-(methanesulfonylmethyl)cyclohexyl]benzene-1,3-diamine

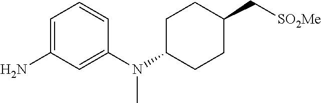 [SMILES: CN(c1cccc(N)c1)[C@H]1CC[C@H](CS(C)(=O)=O)CC1]

General procedure 12 was applied N-methyl-3-nitro-N-[(1r,4r)-4-(methanesulfonylmethyl)cyclohexyl]aniline (300 mg, 0.919 mmol) and palladium on carbon (10%, 90.0 mg) in THF (30 mL) to yield the crude title compound as a brown semi-solid (300 mg, 110%).

### 8-Cyclopentyl-2-[(3-(methyl[(1r,4r)-4-(methanesulfonylmethyl)cyclohexyl]aminophenyl)amino]-5-[2-(triisopropylsilyl)ethynyl]pyrido[2,3-d]pyrimidin-7-one

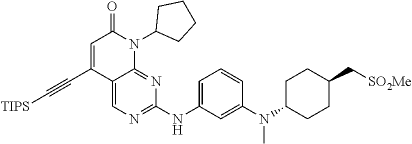

General procedure 13 was applied to 8-cyclopentyl-2-methanesulfonyl-5-[2-(triisopropylsilyl)ethynyl]pyrido[2,3-d]pyrimidin-7-one (140 mg, 0.296 mmol), N¹-methyl-N¹-[(1r,4r)-4-(methanesulfonylmethyl)cyclohexyl]benzene-1,3-diamine (131 mg, 0.444 mmol) and and trifluoroacetic acid (45.3 μL, 0.592 mmol) in 2-butanol (1.5 mL), stirred for 2 hours at 100° C. The residue was purified by reverse phase flash column chromatography eluting with acetonitrile (10-100%) in water (0.1% formic acid), to afford the title compound as a brown solid (77.1 mg, 37%).

### 8-Cyclopentyl-5-ethynyl-2-[(3-(methyl[(1r,4r)-4-(methanesulfonylmethyl)cyclohexyl]aminophenyl)amino]pyrido[2,3-d]pyrimidin-7-one

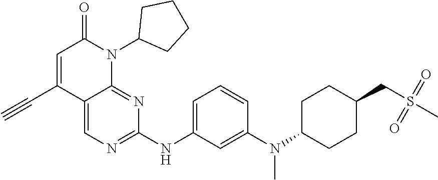 [SMILES: C#Cc1cc(=O)n(C2CCCC2)c2nc(Nc3cccc(N(C)[C@H]4CC[C@H](CS(C)(=O)=O)CC4)c3)ncc12]

General procedure 15 was applied to 8-cyclopentyl-2-[(3-(methyl[(1r,4r)-4-(methanesulfonylmethyl)cyclohexyl]aminophenyl)amino]-5-[2-(triisopropylsilyl)ethynyl]pyrido[2,3-d]pyrimidin-7-one (120 mg, 0.174 mmol) and potassium fluoride (101 mg, 1.74 mmol) in THF (1.2 L), DMF (1.2 mL) and water (120 μL), stirred for 2 hours at 60° C. The residue was purified by reverse phase flash column chromatography eluting with acetonitrile (10-80%) in water (0.1% ammonium formate), to afford the title compound as a yellow solid (24.2 mg, 25%).

(ES, m/z) [M+H]⁺=534.15

¹H-NMR (400 MHz, DMSO-d₆) δ 9.89 (s, 1H), 9.14 (s, 1H), 7.47 (s, 1H), 7.21 (d, J=8.2 Hz, 1H), 7.11 (t, J=8.1 Hz, 1H), 6.88 (s, 1H), 6.46 (dd, J=8.3, 2.4 Hz, 1H), 5.59 (tt, J=6.4, 2.9 Hz, 1H), 5.03 (s, 1H), 3.65-3.52 (m, 1H), 3.06 (d, J=6.2 Hz, 2H), 2.98 (s, 3H), 2.74 (s, 3H), 2.08-1.54 (m, 15H), 1.27 (dd, J=18.8, 7.3 Hz, 2H).

### Example 207

### 1-Fluoro-4-nitro-2-propoxybenzene

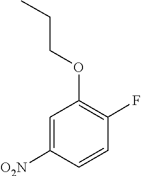

General procedure 22 was applied to 2-fluoro-5-nitrophenol (457 mg, 2.90 mmol), 1-propanol (1.43 mL, 19.1 mmol), DIAD (2.94 mL, 15.28 mmol) and triphenylphosphine (4.00 g, 15.28 mmol) in THF (25 mL). The crude product was purified by flash column chromatography eluting with ethyl acetate (10%) in petroleum ether to afford the title compound as a colourless oil (1.35 g, 53%).

¹H NMR (500 MHz, CDC₃) 57.81-7.74 (2H, m), 7.18-7.10 (1H, m), 4.03 (2H, t, J=6.5 Hz), 1.91-1.80 (2H, m), 1.04 (3H, t, J=7.5 Hz).

### 1-Methyl-4-(4-nitro-2-propoxyphenyl)piperazine

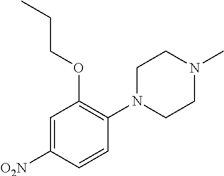 [SMILES: CCCOc1cc([N+](=O)[O-])ccc1N1CCN(C)CC1]

General procedure 11 was applied to 1-Fluoro-2-methoxy-4-nitrobenzene (1.35 g, 6.78 mmol), N-methylpiperazine (2.26 mL, 20.3 mmol) and potassium carbonate (1.41 g, 10.7 mmol) in acetonitrile (13.6 mL). The crude product was purified by flash column chromatography eluting with methanol (10%) in dichloromethane to afford the title compound as a yellow solid (1.90 g, quant.).

¹H NMR (500 MHz, MeOD) δ8.10 (1H, dd, J=8.8 and 2.5 Hz), 7.94 (1H, d, J=2.5 Hz), 7.27 (1H, d, J=8.8 Hz), 4.35 (2H, t, J=6.3 Hz), 3.68-3.64 (4H, m), 3.05-3.01 (4H, m), 2.31-2.21 (2H, m), 1.50 (3H, t, J=7.4 Hz).

### 4-(4-Methylpiperazin-1-yl)-3-propoxyaniline

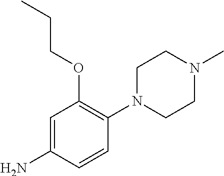 [SMILES: CCCOc1cc(N)ccc1N1CCN(C)CC1]

General procedure 12 was applied to 1-(2-Methoxy-4-nitrophenyl)-4-methylpiperazine (1.90 g, 7.62 mmol) and palladium on carbon (190 mg, 1.79 mmol) in ethanol (20 mL), stirred for 3 hours at room temperature under a hydrogen atmosphere. The resulting mixture was filtered, washing with ethanol (3×2.0 mL). The filtrate was concentrated under reduced pressure to afford the title compound as a brown oil (1.57 g, 83%).

¹H NMR (500 MHz, MeOD) δ6.73 (1H, d, J=8.3 Hz), 6.38 (1H, d, J=2.4 Hz), 6.26 (1H, dd, J=8.3 and 2.4 Hz), 3.87 (2H, t, J=6.4 Hz), 2.99-2.95 (4H, m), 2.61-2.57 (4H, m), 2.31 (3H, s), 1.88-1.74 (2H, m), 1.06 (3H, t, J=7.4 Hz).

### 8-Methyl-2-((4-(4-methylpiperazin-1-yl)-3-propoxyphenyl)amino)-5-((triisopropylsilyl)ethynyl)pyrido[2,3-d]pyrimidin-7(8H)-one

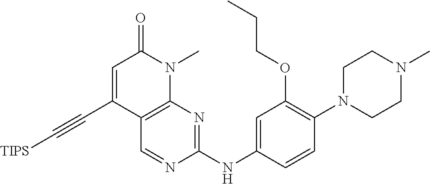

General procedure 13 was applied to 4-(4-methylpiperazin-1-yl)-3-propoxyaniline (475 mg, 1.90 mmol) with 8-methyl-2-(methylsulfonyl)-5-((triisopropylsilyl)ethynyl)pyrido[2,3-d]pyrimidin-7(8H)-one (400 mg, 0.950 mmol) and trifluoroacetic acid (145 μL, 1.90 mmol) in acetonitrile (9.5 mL). The crude material was purified by flash column chromatography eluting with methanol (10%) in dichloromethane to yield the title compound as a yellow solid (405 mg, 73%).

¹H NMR (500 MHz, CDCl₃) δ 8.98-8.67 (1H, m), 8.44-7.96 (1H, m), 7.40-7.36 (1H, m), 7.18-6.95 (1H, m), 6.96-6.78 (1H, m), 6.70-6.50 (1H, m), 4.05-3.80 (2H, m), 3.81-3.57 (5H, m), 3.57-3.32 (2H, m), 3.34-3.18 (2H, m), 3.16-2.95 (2H, m), 2.84 (3H, s), 1.93-1.72 (2H, m), 1.33-0.82 (24H, m).

### 5-Ethynyl-8-methyl-2-((4-(4-methylpiperazin-1-yl)-3-propoxyphenyl)amino)pyrido[2,3-d]pyrimidin-7(8H)-one

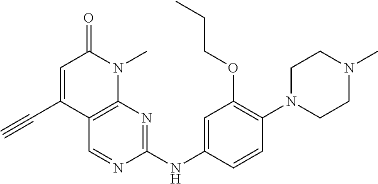 [SMILES: C#Cc1cc(=O)n(C)c2nc(Nc3ccc(N4CCN(C)CC4)c(OCCC)c3)ncc12]

General procedure 15 was applied to 8-methyl-2-((4-(4-methylpiperazin-1-yl)-3-propoxyphenyl)amino)-5-((triisopropylsilyl)ethynyl)pyrido[2,3-d]pyrimidin-7(8H)-one (405 mg, 0.694 mmol) with potassium fluoride (79 mg, 1.38 mmol) in DMF (5.0 mL). The crude material was purified by flash column chromatography eluting with methanol (10%) in dichloromethane to yield the title compound as a yellow solid (130 mg, 43%).

(ES, m/z): [M+1]+=433

¹H NMR (500 MHz, DMSO-d₆) δ 8.74 (1H, s), 7.89 (1H, s), 7.49-7.42 (1H, m), 7.23-7.16 (1H, m), 6.83-6.77 (1H, m), 6.44 (1H, s), 4.40 (1H, s), 3.91 (2H, t, J=6.4 Hz), 3.58 (3H, s), 3.19-3.14 (4H, m), 3.07-2.91 (4H, m), 1.82-1.74 (2H, m), 1.00 (3H, t, J=7.4 Hz).

### Example 208

### 1-Fluoro-2-methoxy-4-nitrobenzene

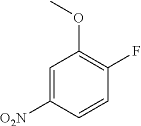

To a stirred solution of 2-fluoro-5-nitrophenol (2.00 g, 12.7 mmol) and potassium carbonate (2.64 g, 19.1 mmol) in DMF (10 mL) was added methyl iodide (1.20 mL, 19.1 mmol) dropwise and left to stir for 18 hours at room temperature. The reaction mixture was diluted in ethyl acetate (30 mL), then washed with water (20 mL), brine (20 mL), dried (MgSO₄) and concentrated under reduced pressure. The crude product was purified by flash column chromatography eluting petroleum ether (100%) to afford the title compound as a white solid (1.34 g, 61%).

¹H NMR (500 MHz, DMSO-d₆) δ 7.84-7.76 (2H, m), 7.20-7.12 (1H, m), 3.95 (3H, s).

### 1-(2-Methoxy-4-nitrophenyl)-4-methylpiperazine

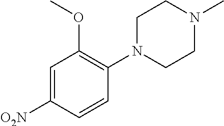 [SMILES: COc1cc([N+](=O)[O-])ccc1N1CCN(C)CC1]

General procedure 11 was applied to 1-fluoro-2-methoxy-4-nitrobenzene (1.34 g, 7.82 mmol), N-methylpiperazine (2.28 mL, 23.5 mmol), potassium carbonate (1.62 g, 11.7 mmol) in acetonitrile (20 mL). The crude product was purified by flash column chromatography eluting with methanol (10%) in dichloromethane to afford the title compound as a yellow solid (1.63 g, 83%).

¹H NMR (500 MHz, DMSO-d₆) 57.68 (1H, dd, J=8.8 and 2.6 Hz), 7.59 (1H, d, J=2.6 Hz), 6.84 (1H, d, J=8.8 Hz), 3.89 (3H, s), 3.24-3.18 (4H, m), 2.60-2.54 (4H, m), 2.32 (3H, s).

### 3-Methoxy-4-(4-methylpiperazin-1-yl)aniline

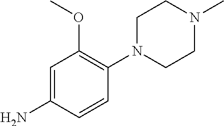 [SMILES: COc1cc(N)ccc1N1CCN(C)CC1]

General procedure 12 was applied to 1-(2-Methoxy-4-nitrophenyl)-4-methylpiperazine (1.63 g, 6.49 mmol) and palladium on carbon (163 mg, 1.53 mmol) in ethanol (20 mL), stirred for 3 hours at room temperature under a hydrogen atmosphere. The resulting mixture was filtered, washing with ethanol (3×2.0 mL). The filtrate was concentrated under reduced pressure to afford the title compound as a brown oil (1.34 g, 61%).

¹H NMR (500 MHz, DMSO-d₆) 56.77 (1H, d, J=8.3 Hz), 6.40 (1H, d, J=2.4 Hz), 6.28 (1H, dd, J=8.3 and 2.4 Hz), 3.77 (3H, s), 3.04-2.91 (4H, m), 2.68-2.53 (4H, m), 2.31 (3H, s).

### 2-((3-Methoxy-4-(4-methylpiperazin-1-yl)phenyl)amino)-8-methyl-5-((triisopropylsilyl)ethynyl)pyrido[2,3-d]pyrimidin-7(8H)-one

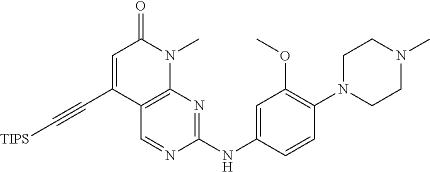

General procedure 13 was applied to 3-methoxy-4-(4-methylpiperazin-1-yl)aniline (420 mg, 1.90 mmol) with 8-methyl-2-(methylsulfonyl)-5-((triisopropylsilyl)ethynyl)pyrido[2,3-d]pyrimidin-7(8H)-one (400 mg, 0.950 mmol) and trifluoroacetic acid (145 μL, 1.90 mmol) in acetonitrile (9.5 mL). The crude material was purified by flash column chromatography eluting with methanol (10%) in dichloromethane to yield the title compound as a yellow solid (490 mg, 93%).

¹H NMR (500 MHz, CDCl₃) δ 8.77 (1H, s), 8.38 (1H, s), 7.38-7.34 (1H, m), 7.13-7.07 (1H, m), 6.53 (1H, s), 3.80 (3H, s), 3.62-3.54 (5H, m), 3.42-3.62 (2H, m), 3.20-3.11 (2H, m), 3.10-3.01 (2H, m), 2.82 (3H, s), 1.11-0.99 (21H, m).

### 5-Ethynyl-2-((3-methoxy-4-(4-methylpiperazin-1-yl)phenyl)amino)-8-methylpyrido[2,3-d]pyrimidin-7(8H)-one

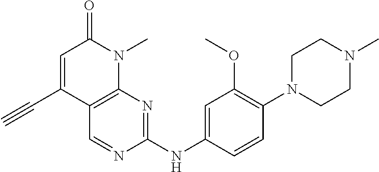 [SMILES: C#Cc1cc(=O)n(C)c2nc(Nc3ccc(N4CCN(C)CC4)c(OC)c3)ncc12]

General procedure 15 was applied to 2-((3-methoxy-4-(4-methylpiperazin-1-yl)phenyl)amino)-8-methyl-5-((triisopropylsilyl)ethynyl)pyrido[2,3-d]pyrimidin-7(8H)-one (490 mg, 0.871 mmol) with potassium fluoride (101 mg, 1.75 mmol) in DMF (5.0 mL). The crude material was purified by flash column chromatography eluting with methanol (10%) in dichloromethane to yield the title compound as a yellow solid (186 mg, 53%)

(ES, m/z): [M+1]+=405

¹H NMR (500 MHz, CDCl₃) δ 8.84 (1H, s), 7.44 (1H, s), 7.37 (1H, d, J=2.5 Hz), 7.09 (1H, dd, J=8.5 and 2.5 Hz), 6.95 (1H, d, J=8.5 Hz), 6.64 (1H, s), 3.91 (3H, s), 3.61 (1H, s), 3.48 (3H, s), 3.12-3.09 (4H, m), 2.66-2.62 (4H, m), 2.37 (3H, s).

### Example 209

### 2-(2-Cyclopentylethoxy)-1-fluoro-4-nitrobenzene

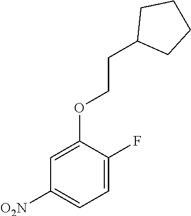 [SMILES: O=[N+]([O-])c1ccc(F)c(OCCC2CCCC2)c1]

General procedure 22 was applied to 2-fluoro-5-nitrophenol (2.00 g, 12.7 mmol), 2-cyclopentylethanol (1.89 mL, 15.3 mmol), DIAD (2.99 mL, 15.3 mmol) and triphenylphosphine (4.06 g, 15.3 mmol) in THF (20 mL). The crude product was purified by flash column chromatography eluting with ethyl acetate (10%) in petroleum ether to afford the title compound as a colourless oil (2.30 g, 71%).

¹H NMR (500 MHz, CDC₃) 67.87-7.82 (2H, m), 7.23-7.15 (1H, m), 4.13 (1H, t, J=6.7 Hz), 2.06-1.93 (1H, m), 1.89-1.80 (2H, m), 1.71-1.48 (4H, m), 1.40-1.11 (4H, m).

### 1-(2-(2-Cyclopentylethoxy)-4-nitrophenyl)-4-methylpiperazine

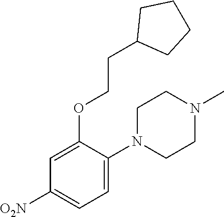 [SMILES: CN1CCN(c2ccc([N+](=O)[O-])cc2OCCC2CCCC2)CC1]

General procedure 11 was applied to 2-(2-cyclopentylethoxy)-1-fluoro-4-nitrobenzene (2.30 g, 9.10 mmol), N-methylpiperazine (3.0 mL, 27.3 mmol), potassium carbonate (1.75 g, 13.6 mmol) in acetonitrile (10 mL). The crude product was purified by flash column chromatography eluting with methanol (10%) in dichloromethane to afford the title compound as a yellow solid (2.97 g, 89%).

¹H NMR (500 MHz, DMSO-d₆) 7.83 (1H, dd, J=8.9 Hz and 2.6 Hz), 7.69 (1H, d, J=2.6 Hz), 7.01 (1H, d, J=8.9 Hz), 4.11 (1H, t, J=6.5 Hz), 3.25-3.16 (4H, m), 2.50-2.44 (4H, m), 2.06-1.93 (1H, m), 1.68-1.60 (4H, m), 1.58-1.50 (2H, m), 1.28-1.25 (2H, m).

### 3-(2-Cyclopentylethoxy)-4-(4-methylpiperazin-1-yl)aniline

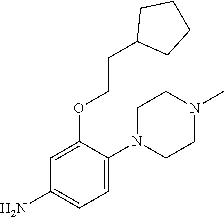 [SMILES: CN1CCN(c2ccc(N)cc2OCCC2CCCC2)CC1]

General procedure 12 was applied to 1-(2-(2-cyclopentylethoxy)-4-nitrophenyl)-4-methylpiperazine (2.97 g, 8.90 mmol) and palladium on carbon (297 mg, 2.79 mmol) in ethanol (50 mL) was stirred for 3 hours at room temperature under a hydrogen atmosphere. The resulting mixture was filtered, washing with ethanol (3×2.0 mL). The filtrate was concentrated under reduced pressure to afford the title compound as a brown oil (1.68 g, 62%).

¹H NMR (500 MHz, DMSO-d₆) 6.70 (1H, d, J=8.4 Hz), 6.35 (1H, d, J=2.4 Hz), 6.23 (1H, dd, J=8.4 and 2.4 Hz), 3.90 (1H, t, J=6.7 Hz), 2.97-2.93 (4H, m), 2.59-2.52 (4H, m).

### 2-((3-(2-Cyclopentylethoxy)-4-(4-methylpiperazin-1-yl)phenyl)amino)-8-methyl-5-((triisopropylsilyl)ethynyl)pyrido[2,3-d]pyrimidin-7(8H)-one

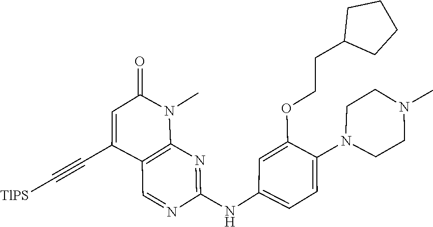

General procedure 13 was applied to 3-(2-cyclopentylethoxy)-4-(4-methylpiperazin-1-yl)aniline (576 mg, 1.90 mmol) with 8-methyl-2-(methylsulfonyl)-5-((triisopropylsilyl)ethynyl)pyrido[2,3-d]pyrimidin-7(8H)-one (400 mg, 0.950 mmol) and trifluoroacetic acid (145 μL, 1.90 mmol) in acetonitrile (9.5 mL). The crude material was purified by flash column chromatography eluting with methanol (10%) in dichloromethane to yield the title compound as a yellow solid (515 mg, 84%).

¹H NMR (500 MHz, CDCl₃) 8.85 (1H, s), 7.83 (1H, br. s), 7.47-7.43 (1H, m), 7.08-7.01 (1H, m), 6.92-6.86 (1H, m), 6.63 (1H, s), 4.03 (2H, t, J=6.9 Hz), 3.73-3.61 (5H, m), 3.52-3.46 (2H, m), 3.24 (2H, t, J=12.3 Hz), 3.07-3.04 (2H, m), 2.87 (3H, s), 2.02-1.52 (11H, m), 1.26-1.08 (21H, m).

### 2-((3-(2-Cyclopentylethoxy)-4-(4-methylpiperazin-1-yl)phenyl)amino)-5-ethynyl-8-methylpyrido[2,3-d]pyrimidin-7(8H)-one

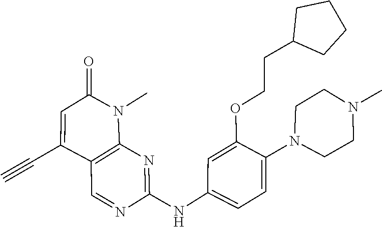 [SMILES: C#Cc1cc(=O)n(C)c2nc(Nc3ccc(N4CCN(C)CC4)c(OCCC4CCCC4)c3)ncc12]

General procedure 15 was applied to 2-((3-(2-cyclopentylethoxy)-4-(4-methylpiperazin-1-yl)phenyl)amino)-8-methyl-5-((triisopropylsilyl)ethynyl)pyrido[2,3-d]pyrimidin-7(8H)-one (515 mg, 0.691 mmol) with potassium fluoride (93 mg, 1.60 mmol) in DMF (5.0 mL). The crude material was purified by flash column chromatography eluting with methanol (10%) in dichloromethane to yield the title compound as a yellow solid (275 mg, 71%),

(ES, m/z): [M+1]+=487

¹H NMR (500 MHz, CDCl₃) 8.84 (1H, s), 7.43-7.40 (1H, m), 7.34-7.31 (1H, m), 7.04 (1H, dd, J=8.5 and 2.3 Hz), 6.95-6.90 (1H, m), 6.64 (1H, s), 4.04 (2H, t, J=6.4 Hz), 3.70 (3H, s), 3.61 (1H, s), 3.14-3.11 (4H, m), 2.65-2.62 (4H, m), 2.38 (3H, s), 2.04-1.94 (1H, m), 1.94-1.80 (2H, m), 1.71-1.53 (6H, s), 1.25-1.14 (2H, s).

### Example 210

### 8-((1-Methyl-1H-pyrazol-3-yl)methyl)-2-(methylthio)-5-((triisopropylsilyl)ethynyl)pyrido[2,3-d]pyrimidin-7(8H)-one

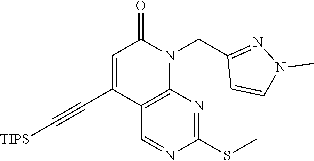

To a stirred solution of 2-(methylthio)-5-((triisopropylsilyl)ethynyl)pyrido[2,3-d]pyrimidin-7(8H)-one (460 mg, 1.23 mmol) and potassium carbonate (205 mg, 1.48 mmol) in DMF (2.5 mL) was added 3-bromomethyl-1-methyl-1H-pyrazole (250 mg, 1.48 mmol) dropwise and left to stir for 18 hours at room temperature. The reaction mixture was diluted in ethyl acetate (10 mL), then washed with water (10 mL), brine (10 mL), dried (MgSO₄) and concentrated under reduced pressure. The crude product was purified by flash column chromatography eluting with ethyl acetate (50%) in petroleum ether to afford the title compound as a white solid (114 mg, 20%).

¹H NMR (500 MHz, CDCl₃) 58.94 (1H, s), 7.21 (1H, d, J=2.3 Hz), 6.80 (1H, s), 6.10 (1H, d, J=2.3 Hz), 5.61 (2H, s), 3.81 (3H, s), 2.64 (3H, s), 1.29-1.07 (21H, m).

### 8-((1-Methyl-1H-pyrazol-3-yl)methyl)-2-(methylsulfonyl)-5-((triisopropylsilyl)ethynyl)pyrido[2,3-d]pyrimidin-7(8H)-one

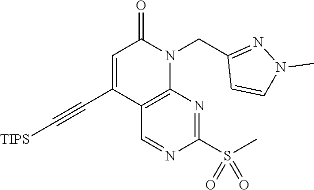

General procedure 10 was applied to 8-((1-methyl-1H-pyrazol-3-yl)methyl)-2-(methylthio)-5-((triisopropylsilyl)ethynyl)pyrido[2,3-d]pyrimidin-7(8H)-one (114 mg, 0.240 mmol) with m-CPBA (126 mg, 0.730 mmol) in dichloromethane (5.0 mL). The crude material was concentrated under reduced pressure to the title compound as a brown solid (124 mg, 100%).

¹H NMR (500 MHz, CDCl₃) 58.92 (1H, s), 7.19 (1H, d, J=2.3 Hz), 6.80 (1H, s), 6.10 (1H, d, J=2.3 Hz), 5.61 (2H, s), 3.76 (3H, s), 2.53 (3H, s), 1.24-1.05 (21H, m).

### 2-((3-(2-Cyclopentylethoxy)-4-(4-methylpiperazin-1-yl)phenyl)amino)-5-ethynyl-8-((1-methyl-1H-pyrazol-3-yl)methyl)pyrido[2,3-d]pyrimidin-7(8H)-one

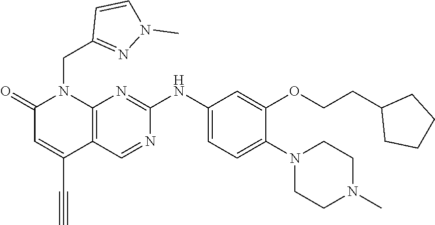 [SMILES: C#Cc1cc(=O)n(Cc2ccn(C)n2)c2nc(Nc3ccc(N4CCN(C)CC4)c(OCCC4CCCC4)c3)ncc12]

General procedure 13 was applied to 3-(2-cyclopentylethoxy)-4-(4-methylpiperazin-1-yl)aniline (113 mg, 0.37 mmol) with 8-((1-methyl-1H-pyrazol-3-yl)methyl)-2-(methylsulfonyl)-5-((triisopropylsilyl)ethynyl)pyrido[2,3-d]pyrimidin-7(8H)-one (124 mg, 0.25 mmol) and trifluoroacetic acid (28 μL, 0.37 mmol) in acetonitrile (2.5 mL). The crude material was purified by flash column chromatography eluting with methanol (10%) in dichloromethane to give a crude material.

General procedure 15 was applied to the crude material (153 mg, 0.210 mmol) with potassium fluoride (155 mg, 2.70 mmol) in DMF (5.0 mL). The crude material was purified by flash column chromatography eluting with methanol (10%) in dichloromethane to yield the title compound as a yellow solid (70.0 mg, 57%).

(ES, m/z): [M+1]+=567

¹H NMR (500 MHz, CDCl₃) δ 8.82 (1H, s), 7.96 (1H, s), 7.34-7.28 (1H, m), 7.22-7.15 (2H, m), 6.89 (1H, d, J=8.6 Hz), 6.64 (1H, s), 6.18-6.13 (1H, m), 5.59 (2H, s), 4.02-3.90 (2H, m), 3.81 (3H, s), 3.61 (1H, s), 3.16-3.05 (4H, m), 2.71-2.59 (4H, m), 2.37 (3H, s), 2.00-1.89 (1H, m), 1.84-1.76 (4H, m), 1.68-1.61 (2H, m), 1.58-1.50 (2H, m), 1.20-1.11 (2H, m).

### Example 211

### 2-(Cyclopentylmethoxy)-1-fluoro-4-nitrobenzene

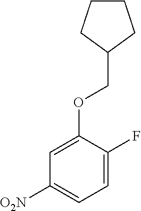 [SMILES: O=[N+]([O-])c1ccc(F)c(OCC2CCCC2)c1]

General procedure 22 was applied to 2-fluoro-5-nitrophenol (500 mg, 3.18 mmol), cyclopentanemethanol (514 μL, 4.77 mmol), DIAD (750 μL, 3.82 mmol) and triphenylphosphine (1.00 g, 3.82 mmol) in THF (6.4 mL). The crude product was purified by flash column chromatography eluting with ethyl acetate (20%) in petroleum ether to afford the title compound as a colourless oil (530 mg, 70%).

¹H NMR (500 MHz, CDCl₃) δ7.87-8.74 (2H, m), 7.25-7.15 (1H, m), 3.98 (2H, d, J=6.9 Hz), 2.47-2.42 (1H, m), 1.94-1.83 (2H, m), 1.76-1.55 (4H, m), 1.45-1.34 (2H, m).

### 1-(2-(Cyclopentylmethoxy)-4-nitrophenyl)-4-methylpiperazine

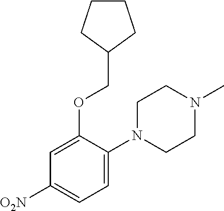 [SMILES: CN1CCN(c2ccc([N+](=O)[O-])cc2OCC2CCCC2)CC1]

General procedure 11 was applied to 2-(cyclopentylmethoxy)-1-fluoro-4-nitrobenzene (530 mg, 2.22 mmol), N-methylpiperazine (491 μL, 4.43 mmol), potassium carbonate (367 mg, 2.66 mmol) in acetonitrile (4.5 mL) to afford the title compound as a yellow solid (653 mg, 92%). No further purification was required.

¹H NMR (500 MHz, CDCl₃) δ7.84 (1H, dd, J=8.8 and 2.5 Hz), 7.68 (1H, d, J=2.5 Hz), 6.85 (1H, d, J=8.8 Hz), 3.95 (2H, t, J=7.0 Hz), 3.31-3.27 (4H, m), 2.62-2.57 (4H, m), 2.51-2.38 (1H, m), 2.36 (3H, s), 1.93-1.83 (2H, m), 1.74-1.58 (4H, m), 1.45-1.34 (2H, m).

### 3-(Cyclopentylmethoxy)-4-(4-methylpiperazin-1-yl)aniline

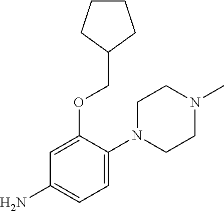 [SMILES: CN1CCN(c2ccc(N)cc2OCC2CCCC2)CC1]

General procedure 12 was applied to 1-(2-(cyclopentylmethoxy)-4-nitrophenyl)-4-methylpiperazine (653 mg, 2.04 mmol) and palladium on carbon (65 mg, 0.61 mmol) in ethanol (20 mL) was stirred for 18 h at room temperature under a hydrogen atmosphere. The resulting mixture was filtered, washing with ethanol (3×2.0 mL). The filtrate was concentrated under reduced pressure to afford the title compound as a brown oil (503 g, 85%).

¹H NMR (500 MHz, CDCl₃) 56.78 (1H, d, J=8.6 Hz), 6.43 (1H, d, J=2.3 Hz), 6.31 (1H, dd, J=8.6 and 2.3 Hz), 3.86 (2H, d, J=6.9 Hz), 3.04-3.01 (4H, m), 2.65-2.61 (4H, m), 2.49-2.37 (1H, m), 2.36 (3H, s), 1.99-1.86 (2H, m), 1.79-1.60 (4H, m), 1.50-1.40 (2H, m).

### 2-((3-(Cyclopentylmethoxy)-4-(4-methylpiperazin-1-yl)phenyl)amino)-8-methyl-5-((triisopropylsilyl)ethynyl)pyrido[2,3-d]pyrimidin-7(8H)-one

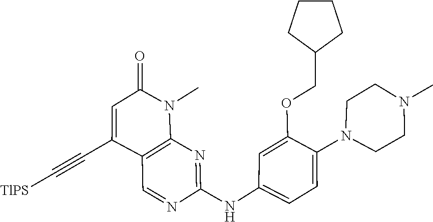

General procedure 13 was applied to 3-(cyclopentylmethoxy)-4-(4-methylpiperazin-1-yl)aniline (503 mg, 1.74 mmol) with 8-methyl-2-(methylsulfonyl)-5-((triisopropylsilyl)ethynyl)pyrido[2,3-d]pyrimidin-7(8H)-one (414 mg, 0.99 mmol) and trifluoroacetic acid (134 μL, 1.74 mmol) in acetonitrile (9.8 mL). The crude material was purified by flash column chromatography eluting with methanol (10%) in dichloromethane to yield the title compound as a yellow solid (446 mg, 72%).

¹H NMR (500 MHz, CDCl₃) δ 8.83 (1H, s), 8.08-8.04 (1H, m), 7.51-7.45 (1H, m), 7.02 (1H, d, J=8.2 Hz), 6.88-6.83 (1H, m), 6.60 (1H, s), 3.87 (2H, d, J=7.2 Hz), 3.67 (3H, s), 3.64-3.59 (2H, m), 3.51-3.45 (2H, m), 3.26-3.18 (2H, m), 3.08-3.00 (2H, m), 2.85 (3H, s), 2.46-2.33 (1H, m), 1.87-1.77 (2H, m), 1.68-1.55 (4H, m), 1.36-1.25 (2H, m), 1.24-1.03 (21H, m).

### 2-((3-(Cyclopentylmethoxy)-4-(4-methylpiperazin-1-yl)phenyl)amino)-5-ethynyl-8-methylpyrido[2,3-d]pyrimidin-7(8H)-one

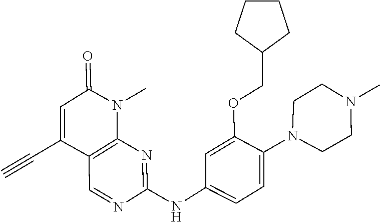 [SMILES: C#Cc1cc(=O)n(C)c2nc(Nc3ccc(N4CCN(C)CC4)c(OCC4CCCC4)c3)ncc12]

General procedure 15 was applied to 2-((3-(cyclopentylmethoxy)-4-(4-methylpiperazin-1-yl)phenyl)amino)-8-methyl-5-((triisopropylsilyl)ethynyl)pyrido[2,3-d]pyrimidin-7(8H)-one (446 mg, 0.71 mmol) with potassium fluoride (206 mg, 3.55 mmol) in DMF (5.0 mL). The crude material was purified by flash column chromatography eluting with methanol (10%) in dichloromethane to yield the title compound as a yellow solid (149 mg, 44%).

(ES, m/z): [M+1]+=473

¹H NMR (500 MHz, CDCl₃) δ 8.83 (1H, s), 7.49-7.44 (1H, m), 7.41-7.37 (1H, m), 7.01 (1H, dd, J=8.5 and 2.4 Hz), 6.91 (1H, d, J=8.5 Hz), 6.64 (1H, s), 3.92 (2H, d, J=7.1 Hz), 3.71 (3H, s), 3.61 (1H, s), 3.15-3.11 (4H, m), 2.65-2.61 (4H, m), 2.53-2.40 (1H, m), 2.37 (3H, s), 1.94-1.83 (2H, m), 1.71-1.58 (4H, m), 1.42-1.32 (2H, m).

### Example 212

### 2-(3-Cyclopentylpropoxy)-1-fluoro-4-nitrobenzene

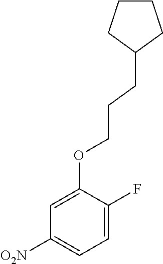 [SMILES: O=[N+]([O-])c1ccc(F)c(OCCCC2CCCC2)c1]

General procedure 22 was applied to 2-fluoro-5-nitrophenol (500 mg, 3.18 mmol), 3-cyclopentylpropan-1-ol (661 μL, 4.77 mmol), DIAD (750 μL, 3.82 mmol) and triphenylphosphine (1.00 g, 3.82 mmol) in THF (6.4 mL). The crude product was purified by flash column chromatography eluting petroleum ether (100%) to afford the title compound as a colourless oil (814 mg, 96%).

¹H NMR (500 MHz, CDCl₃) δ7.91 (1H, dd, J=7.5 and 2.8 Hz), 7.85-7.78 (1H, m), 7.25-7.18 (1H, m), 4.09 (1H, t, J=6.4 Hz), 1.95-1.78 (5H, m), 1.71-1.49 (6H, m), 1.18-1.11 (2H, m).

### 1-(2-(3-Cyclopentylpropoxy)-4-nitrophenyl)-4-methylpiperazine

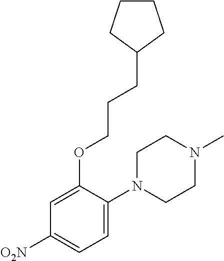 [SMILES: CN1CCN(c2ccc([N+](=O)[O-])cc2OCCCC2CCCC2)CC1]

General procedure 11 was applied to 2-(3-cyclopentylpropoxy)-1-fluoro-4-nitrobenzene (814 mg, 3.04 mmol), N-methylpiperazine (675 μL, 6.09 mmol), potassium carbonate (504 mg, 3.65 mmol) in acetonitrile (6.1 mL) to afford the title compound as a yellow oil (920 mg, 87%). No further purification was required.

¹H NMR (500 MHz, CDCl₃) δ7.87 (1H, dd, J=8.8 and 2.5 Hz), 7.71 (1H, d, J=2.1 Hz), 6.89 (1H, d, J=8.8 Hz), 4.11 (1H, t, J=6.4 Hz), 1.93-1.78 (5H, m), 1.71-1.48 (6H, m), 1.13-1.10 (2H, m).

### 3-(3-Cyclopentylpropoxy)-4-(4-methylpiperazin-1-yl)aniline

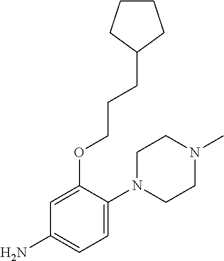 [SMILES: CN1CCN(c2ccc(N)cc2OCCCC2CCCC2)CC1]

General procedure 12 was applied to 1-(2-(3-cyclopentylpropoxy)-4-nitrophenyl)-4-methylpiperazine (920 mg, 2.65 mmol) and palladium on carbon (92 mg, 0.86 mmol) in ethanol (20 mL) was stirred for 18 h at room temperature under a hydrogen atmosphere. The resulting mixture was filtered, washing with ethanol (3×2.0 mL). The filtrate was concentrated under reduced pressure to afford the title compound as a brown oil (789 mg, 94%).

¹H NMR (500 MHz, MeOD) δ 6.76 (1H, d, J=8.4 Hz), 6.40 (1H, d, J=2.4 Hz), 6.29 (1H, dd, J=8.3 and 2.4 Hz), 3.94 (2H, t, J=6.3 Hz), 3.01-2.95 (4H, m), 2.64-2.60 (4H, m), 2.34 (3H, s), 1.88-1.78 (5H, m), 1.69-1.50 (6H, m), 1.32-1.09 (2H, m).

### 2-((3-(3-Cyclopentylpropoxy)-4-(4-methylpiperazin-1-yl)phenyl)amino)-8-methyl-5-((triisopropylsilyl)ethynyl)pyrido[2,3-d]pyrimidin-7(8H)-one

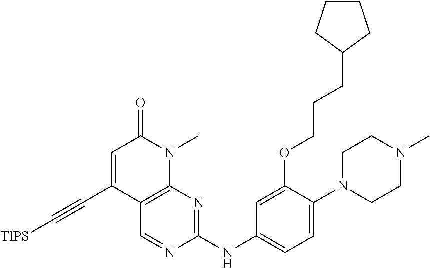

General procedure 13 was applied to 3-(3-cyclopentylpropoxy)-4-(4-methylpiperazin-1-yl)aniline (471 mg, 1.48 mmol) with 8-methyl-2-(methylsulfonyl)-5-((triisopropylsilyl)ethynyl)pyrido[2,3-d]pyrimidin-7(8H)-one (354 mg, 0.84 mmol) and trifluoroacetic acid (135 μL, 1.48 mmol) in acetonitrile (8.4 mL). The crude material was purified by flash column chromatography eluting with methanol (10%) in dichloromethane to yield the title compound as a yellow solid (343 mg, 62%).

¹H NMR (500 MHz, CDCl₃) δ 8.85 (1H, s), 8.23-8.05 (1H, m), 7.43 (1H, s), 7.14-7.09 (1H, m), 6.92-6.87 (1H, m), 6.64 (1H, s), 4.02 (2H, t, J=6.2 Hz), 3.71-3.62 (5H, m), 3.54-3.48 (2H, m), 3.29-3.20 (2H, m), 3.12-3.04 (2H, m), 2.88 (3H, s), 1.91-1.74 (5H, m), 1.66-1.42 (6H, m), 1.26-1.01 (23H, m).

### 2-((3-(3-Cyclopentylpropoxy)-4-(4-methylpiperazin-1-yl)phenyl)amino)-5-ethynyl-8-methylpyrido[2,3-d]pyrimidin-7(8H)-one

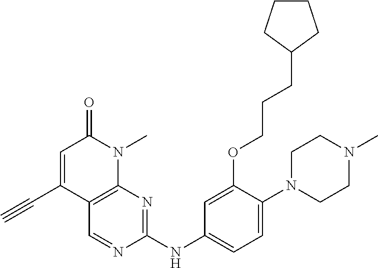 [SMILES: C#Cc1cc(=O)n(C)c2nc(Nc3ccc(N4CCN(C)CC4)c(OCCCC4CCCC4)c3)ncc12]

General procedure 15 was applied to 2-((3-(3-cyclopentylpropoxy)-4-(4-methylpiperazin-1-yl)phenyl)amino)-8-methyl-5-((triisopropylsilyl)ethynyl)pyrido[2,3-d]pyrimidin-7(8H)-one (343 mg, 0.52 mmol) with potassium fluoride (151 mg, 2.61 mmol) in DMF (5.0 mL). The crude material was purified by flash column chromatography eluting with methanol (10%) in dichloromethane to yield the title compound as a yellow solid (112 mg, 43%).

(ES, m/z): [M+1]+=501

¹H NMR (500 MHz, CDC₃) δ 8.81 (1H, s), 7.84-7.71 (1H, s), 7.39-7.33 (1H, s), 7.06 (1H, dd, J=7.5 and 2.6 Hz), 6.93-6.88 (1H, m), 6.62 (1H, s), 4.00 (2H, t, J=6.4 Hz), 3.68 (3H, s), 3.60 (1H, s), 3.21-3.03 (4H, m), 2.71-2.56 (4H, m), 1.91-1.73 (5H, m), 1.66-1.43 (6H, m), 1.18-1.06 (2H, m).

### Example 213

### 2-(2-Cyclohexylethoxy)-1-fluoro-4-nitrobenzene

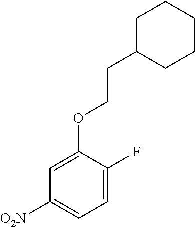 [SMILES: O=[N+]([O-])c1ccc(F)c(OCCC2CCCCC2)c1]

General procedure 22 was applied to 2-fluoro-5-nitrophenol (1.00 g, 6.37 mmol), 2-cyclohexylethanol (1.32 mL, 9.45 mmol), DIAD (1.50 mL, 7.64 mmol) and triphenylphosphine (2.00 g, 7.64 mmol) in THF (12.8 mL). The crude product was purified by flash column chromatography eluting with ethyl acetate (20%) in petroleum ether to afford the title compound as a colourless oil (1.50 g, 89%).

¹H NMR (500 MHz, CDC₃) 57.76-7.65 (2H, m), 7.10-7.02 (1H, m), 4.03 (2H, t, J=6.8 Hz), 1.70-1.53 (7H, m), 1.49-1.35 (1H, m), 1.22-0.99 (3H, m), 0.95-0.82 (2H, m).

### 1-(2-(2-Cyclohexylethoxy)-4-nitrophenyl)-4-methylpiperazine

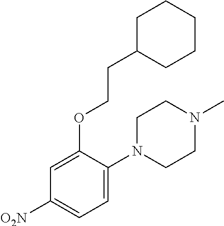 [SMILES: CN1CCN(c2ccc([N+](=O)[O-])cc2OCCC2CCCCC2)CC1]

General procedure 11 was applied to 2-(2-cyclohexylethoxy)-1-fluoro-4-nitrobenzene (1.50 g, 5.66 mmol), N-methylpiperazine (1.25 mL, 11.32 mmol), potassium carbonate (930 mg, 6.80 mmol) in acetonitrile (11.3 mL). The crude product was purified by flash column chromatography eluting with methanol (10%) in dichloromethane to afford the title compound as a yellow oil (1.73 mg, 88%).

¹H NMR (500 MHz, CDCl₃) δ 7.84 (1H, dd, J=8.8 and 2.5 Hz), 7.69 (1H, d, J=2.5 Hz), 6.86 (1H, d, J=8.6 Hz), 4.10 (2H, t, J=6.8 Hz), 3.30-3.25 (4H, m), 2.62-2.57 (4H, m), 2.37 (3H, s), 1.82-1.48 (8H, m), 1.31-1.15 (3H, m), 1.06-0.96 (2H, m).

### 3-(2-Cyclohexylethoxy)-4-(4-methylpiperazin-1-yl)aniline

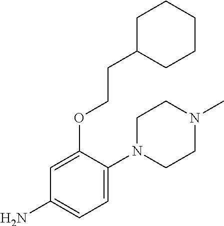 [SMILES: CN1CCN(c2ccc(N)cc2OCCC2CCCCC2)CC1]

General procedure 12 was applied to 1-(2-(2-cyclohexylethoxy)-4-nitrophenyl)-4-methylpiperazine (1.73 g, 2.04 mmol) and palladium on carbon (173 mg, 1.63 mmol) in ethanol (20 mL) was stirred for 18 h at room temperature under a hydrogen atmosphere. The resulting mixture was filtered, washing with ethanol (3×2.0 mL). The filtrate was concentrated under reduced pressure to afford the title compound as a brown oil (1.47 g, 93%).

¹H NMR (500 MHz, MeOD) δ6.75 (1H, d, J=8.4 Hz), 6.42 (1H, d, J=2.4 Hz), 6.28 (1H, d, J=8.4 and 2.4 Hz), 3.99 (2H, t, J=6.5 Hz), 3.02-2.93 (4H, m), 2.68-2.58 (4H, m), 2.34 (3H, s), 1.84-1.57 (8H, m), 1.34-1.16 (3H, m), 1.09-0.95 (2H, m).

### 2-((3-(2-Cyclohexylethoxy)-4-(4-methylpiperazin-1-yl)phenyl)amino)-8-methyl-5-((triisopropylsilyl)ethynyl)pyrido[2,3-d]pyrimidin-7(8H)-one

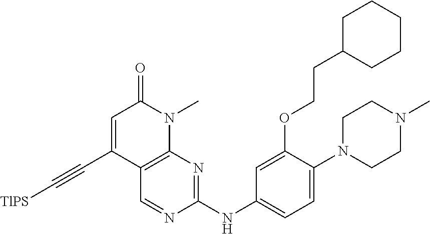

General procedure 13 was applied to 3-(2-cyclohexylethoxy)-4-(4-methylpiperazin-1-yl)aniline (232 mg, 0.73 mmol) with 8-methyl-2-(methylsulfonyl)-5-((triisopropylsilyl)ethynyl)pyrido[2,3-d]pyrimidin-7(8H)-one (256 mg, 0.61 mmol) and trifluoroacetic acid (56 μL, 0.73 mmol) in acetonitrile (6.1 mL). The crude material was purified by flash column chromatography eluting with methanol (10%) in dichloromethane to yield the title compound as a yellow solid (253 mg, 63%).

¹H NMR (500 MHz, CDCl₃) δ 8.83 (1H, s), 8.03 (1H, m), 7.43-7.39 (1H, m), 7.07 (1H, dd, J=8.6 and 2.3 Hz), 6.86 (1H, d, J=8.6 Hz), 6.60 (1H, s), 4.02 (2H, t, J=6.8 Hz), 3.66 (3H, s), 3.64-3.59 (2H, m), 3.49-3.43 (2H, m), 3.25-3.16 (2H, m), 3.09-2.99 (2H, m), 2.85 (3H, s), 1.75-1.38 (8H, m), 1.22-0.94 (26H, m).

### 2-((3-(2-Cyclohexylethoxy)-4-(4-methylpiperazin-1-yl)phenyl)amino)-5-ethynyl-8-methylpyrido[2,3-d]pyrimidin-7(8H)-one

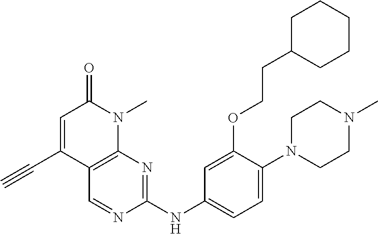 [SMILES: C#Cc1cc(=O)n(C)c2nc(Nc3ccc(N4CCN(C)CC4)c(OCCC4CCCCC4)c3)ncc12]

General procedure 15 was applied to 2-((3-(2-cyclohexylethoxy)-4-(4-methylpiperazin-1-yl)phenyl)amino)-8-methyl-5-((triisopropylsilyl)ethynyl)pyrido[2,3-d]pyrimidin-7(8H)-one (253 mg, 0.38 mmol) with potassium fluoride (223 mg, 3.85 mmol) in DMF (5.0 mL). The crude material was purified by flash column chromatography eluting with methanol (10%) in dichloromethane to yield the title compound as a yellow solid (80 mg, 42%).

(ES, m/z): [M+1]+=501

¹H NMR (500 MHz, CDCl₃) δ 8.80 (1H, s), 7.93-7.90 (1H, m), 7.38-7.34 (1H, m), 7.06 (1H, dd, J=8.5 and 2.4 Hz), 6.89 (1H, d, J=8.5 Hz), 6.61 (1H, s), 4.03 (2H, t, J=6.7 Hz), 3.67 (3H, s), 3.61 (1H, s), 3.12-3.08 (4H, m), 2.65-2.61 (4H, m), 2.36 (3H, s), 1.80-1.63 (8H, m), 1.57-1.47 (1H, m), 1.28-1.11 (3H, m), 1.01-0.91 (2H, m).

### Example 214

### 1-Methyl-4-(4-nitro-2-phenethoxyphenyl)piperazine

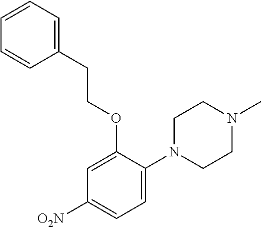 [SMILES: CN1CCN(c2ccc([N+](=O)[O-])cc2OCCc2ccccc2)CC1]

To a stirred solution of 2-(4-methylpiperazin-1-yl)-5-nitrophenol (340 mg, 1.43 mmol) and potassium carbonate (297 mg, 7.64 mmol) in DMF (10 mL) was added 2-bromoethylbenzene (585 μL, 4.30 mmol) dropwise and left to stir for 2 hours at 70° C. The reaction mixture was diluted with ethyl acetate (20 mL), then washed with water (10 mL), brine (10 mL), dried (MgSO₄) and concentrated under reduced pressure. The crude product was purified by flash column chromatography eluting with methanol (10%) in dichloromethane to afford the title compound as a yellow solid (398 mg, 82%).

¹H NMR (500 MHz, CDCl₃) δ7.84 (1H, dd, J=8.8 and 2.5 Hz), 7.71 (1H, d, J=2.5 Hz), 7.37-7.25 (5H, m), 6.85 (1H, d, J=8.8 Hz), 4.34 (1H, t, J=6.4 Hz), 3.19 (1H, t, J=6.4 Hz), 3.16-3.11 (4H, m), 2.54-2.49 (4H, m), 2.36 (3H, s).

### 4-(4-Methylpiperazin-1-yl)-3-phenethoxyaniline

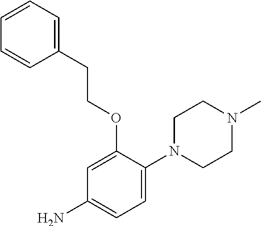 [SMILES: CN1CCN(c2ccc(N)cc2OCCc2ccccc2)CC1]

General procedure 12 was applied to 1-methyl-4-(4-nitro-2-phenethoxyphenyl)piperazine (398 mg, 1.17 mmol) and palladium on carbon (39.0 mg, 0.371 mmol) in ethanol (20 mL) was stirred for 18 hours at room temperature under a hydrogen atmosphere. The resulting mixture was filtered, washing with ethanol (3×2.0 mL). The filtrate was concentrated under reduced pressure to afford the title compound as a brown oil (363 mg, 100%).

¹H NMR (500 MHz, MeOD) δ 7.15-6.99 (5H, m), 6.53 (1H, d, J=8.3 Hz), 6.24 (1H, d, J=2.4 Hz), 6.09 (1H, dd, J=8.3 and 2.4 Hz), 4.07-3.96 (2H, m), 2.94-2.83 (2H, m), 2.70-2.62 (4H, m), 2.56-2.49 (4H, m), 2.30 (3H, s).

### 8-Methyl-2-((4-(4-methylpiperazin-1-yl)-3-phenethoxyphenyl)amino)-5-((triisopropylsilyl)ethynyl)pyrido[2,3-d]pyrimidin-7(8H)-one

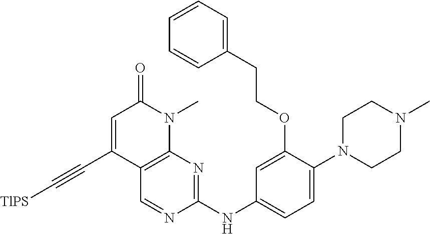

General procedure 13 was applied to 4-(4-methylpiperazin-1-yl)-3-phenethoxyaniline (259 mg, 0.830 mmol) with 8-methyl-2-(methylsulfonyl)-5-((triisopropylsilyl)ethynyl)pyrido[2,3-d]pyrimidin-7(8H)-one (398 mg, 0.951 mmol) and trifluoroacetic acid (87.0 μL, 1.14 mmol) in acetonitrile (9.5 mL). The crude material was purified by flash column chromatography eluting with methanol (10%) in dichloromethane to yield the title compound as a yellow solid (139 mg, 25%).

¹H NMR (500 MHz, CDCl₃) δ 8.85 (1H, s), 8.08-7.96 (1H, m), 7.46 (1H, d, J=2.3 Hz), 7.36-7.22 (5H, m), 7.13 (1H, dd, J=8.6 and 2.3 Hz), 6.89 (1H, d, J=8.6 Hz), 6.64 (1H, s), 4.35 (2H, t, J=6.5 Hz), 3.66 (3H, s), 3.50-3.42 (2H, m), 3.28-3.22 (2H, m), 3.22-3.16 (2H, m), 3.13-3.06 (2H, m), 2.91-2.76 (5H, m), 1.25-1.08 (21H, m).

### 5-Ethynyl-8-methyl-2-((4-(4-methylpiperazin-1-yl)-3-phenethoxyphenyl)amino)pyrido[2,3-d]pyrimidin-7(8H)-one

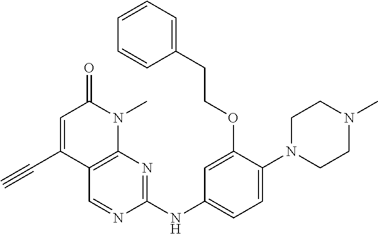 [SMILES: C#Cc1cc(=O)n(C)c2nc(Nc3ccc(N4CCN(C)CC4)c(OCCc4ccccc4)c3)ncc12]

General procedure 15 was applied to 8-methyl-2-((4-(4-methylpiperazin-1-yl)-3-phenethoxyphenyl)amino)-5-((triisopropylsilyl)ethynyl)pyrido[2,3-d]pyrimidin-7(8H)-one (139 mg, 0.214 mmol) with potassium fluoride (124 mg, 2.14 mmol) in DMF (5.0 mL). The crude material was purified by flash column chromatography eluting with methanol (10%) in dichloromethane to yield the title compound as a yellow solid (35 mg, 34%).

(ES, m/z): [M+1]+=495

¹H NMR (500 MHz, CDCl₃) δ 8.66 (1H, s), 7.44-7.41 (1H, m), 7.23 (1H, d, J=2.3 Hz), 7.17-7.05 (5H, m), 6.90 (1H, dd, J=8.6 and 2.3 Hz), 6.74 (1H, d, J=8.6 Hz), 6.46 (1H, s), 4.10 (2H, t, J=6.3 Hz), 3.47 (3H, s), 3.44 (1H, s), 3.02 (2H, t, J=6.3 Hz), 2.89-2.79 (4H, m), 2.46-2.37 (4H, m), 2.21 (3H, s).

### Example 215

### 2-(Cyclopentyloxy)-1-fluoro-4-nitrobenzene

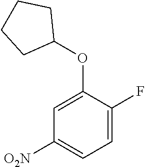 [SMILES: O=[N+]([O-])c1ccc(F)c(OC2CCCC2)c1]

To a stirred solution of 2-fluoro-5-nitrophenol (1.00 g, 6.37 mmol) and potassium carbonate (1.06 g, 7.64 mmol) in DMF (12.7 mL) was added iodocyclopentane (1.10 mL, 9.56 mmol) dropwise and left to stir for 18 hours at 70° C. The reaction mixture was diluted in ethyl acetate (20 mL), then washed with water (10 mL), brine (10 mL), dried (MgSO₄) and concentrated under reduced pressure. The crude product was purified by flash column chromatography eluting with ethyl acetate (10%) in petroleum ether to afford the title compound as a white solid (693 g, 48%).

¹H NMR (500 MHz, CDCl₃) δ7.79-7.69 (2H, m), 7.12-7.08 (1H, m), 4.85-4.78 (1H, m), 2.02-1.72 (6H, m), 1.69-1.50 (2H, m).

### 1-(2-(Cyclopentyloxy)-4-nitrophenyl)-4-methylpiperazine

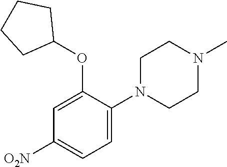 [SMILES: CN1CCN(c2ccc([N+](=O)[O-])cc2OC2CCCC2)CC1]

General procedure 11 was applied to 2-(cyclopentyloxy)-1-fluoro-4-nitrobenzene (693 mg, 3.00 mmol), N-methylpiperazine (684 μL, 6.15 mmol), potassium carbonate (510 mg, 3.69 mmol) in acetonitrile (6.2 mL) to afford the title compound as a yellow oil (884 mg, 94%). No further purification was required.

¹H NMR (500 MHz, CDCl₃) δ 7.66 (1H, dd, J=8.8 and 2.5 Hz), 7.54 (1H, d, J=2.5 Hz), 6.70 (1H, d, J=8.8 Hz), 4.78-4.72 (1H, m), 3.15-3.09 (4H, m), 2.47-2.42 (4H, m), 2.22 (3H, s), 1.93-1.73 (4H, m), 1.71-1.64 (2H, m), 1.62-1.52 (2H, m).

### 3-(Cyclopentyloxy)-4-(4-methylpiperazin-1-yl)aniline

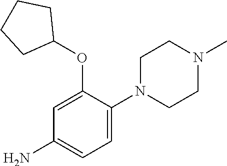 [SMILES: CN1CCN(c2ccc(N)cc2OC2CCCC2)CC1]

General procedure 12 was applied to 1-(2-(cyclopentyloxy)-4-nitrophenyl)-4-methylpiperazine (884 mg, 2.89 mmol) and palladium on carbon (88.0 mg, 0.621 mmol) in ethanol (20 mL) was stirred for 18 hours at room temperature under a hydrogen atmosphere. The resulting mixture was filtered, washing with ethanol (3×2.0 mL). The filtrate was concentrated under reduced pressure to afford the title compound as a brown oil (398 mg, 50%).

¹H NMR (500 MHz, MeOD) δ 6.76 (1H, d, J=8.6 Hz), 6.42 (1H, d, J=2.4 Hz), 6.29 (1H, dd, J=8.6 and 2.4 Hz), 4.84-4.77 (1H, m), 3.04-2.98 (4H, m), 2.79-2.73 (4H, m), 2.46 (3H, s), 1.99-1.74 (6H, m), 1.74-1.61 (2H, m).

### 2-((3-(Cyclopentyloxy)-4-(4-methylpiperazin-1-yl)phenyl)amino)-8-methyl-5-((triisopropylsilyl)ethynyl)pyrido[2,3-d]pyrimidin-7(8H)-one

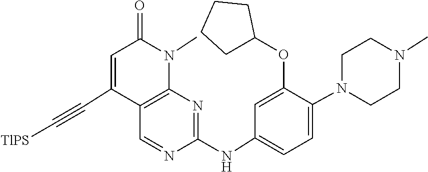

General procedure 13 was applied to 3-(cyclopentyloxy)-4-(4-methylpiperazin-1-yl)aniline (280 mg, 1.01 mmol) with 8-methyl-2-(methylsulfonyl)-5-((triisopropylsilyl)ethynyl)pyrido[2,3-d]pyrimidin-7(8H)-one (198 mg, 0.471 mmol) and trifluoroacetic acid (175 μL, 2.16 mmol) in acetonitrile (4.7 mL). The crude material was purified by flash column chromatography eluting with methanol (10%) in dichloromethane to yield the title compound as a yellow solid (247 mg, 85%).

¹H NMR (500 MHz, CDCl₃) δ 8.87 (1H, s), 8.12-8.08 (1H, m), 7.27-7.22 (1H, m), 7.14-7.10 (1H, m), 6.87-6.84 (1H, m), 6.63 (1H, s), 4.80-4.77 (1H, m), 3.73-3.52 (5H, m), 3.44-3.40 (2H, m), 3.22-3.19 (2H, m), 3.05-3.02 (2H, m), 2.85 (3H, s), 1.97-1.61 (8H, m), 1.19-1.06 (21H, m).

### 2-((3-(Cyclopentyloxy)-4-(4-methylpiperazin-1-yl)phenyl)amino)-5-ethynyl-8-methylpyrido[2,3-d]pyrimidin-7(8H)-one

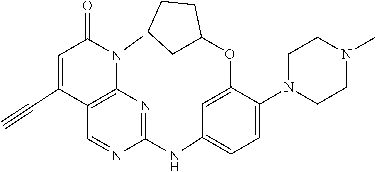 [SMILES: C#Cc1cc(=O)n(C)c2nc(Nc3ccc(N4CCN(C)CC4)c(OC4CCCC4)c3)ncc12]

General procedure 15 was applied to 2-((3-(cyclopentyloxy)-4-(4-methylpiperazin-1-yl)phenyl)amino)-8-methyl-5-((triisopropylsilyl)ethynyl)pyrido[2,3-d]pyrimidin-7(8H)-one (247 mg, 0.401 mmol) with potassium fluoride (233 mg, 4.01 mmol) in DMF (5.0 mL). The crude material was purified by flash column chromatography eluting with methanol (10%) in dichloromethane to yield the title compound as a yellow solid (82 mg, 45%).

(ES, m/z): [M+1]+=459

¹H NMR (500 MHz, CDCl₃) δ 8.82 (1H, s), 7.89-7.86 (1H, m), 7.30-7.23 (1H, m), 7.15 (1H, dd, J=8.8 and 2.4 Hz), 6.91 (1H, d, J=8.8 Hz), 6.63 (1H, s), 4.89-4.82 (1H, m), 3.69 (3H, s), 3.63 (1H, s), 3.13-3.09 (4H, m), 2.66-2.62 (4H, m), 2.37 (3H, s), 1.98-1.90 (4H, m), 1.86-1.76 (2H, m), 1.72-1.61 (2H, m).

### Example 216

### 1-Fluoro-2-(isopentyloxy)-4-nitrobenzene

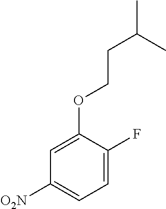 [SMILES: CC(C)CCOc1cc([N+](=O)[O-])ccc1F]

General procedure 22 was applied to 2-fluoro-5-nitrophenol (500 mg, 3.18 mmol), 3-methylbutan-1-ol (519 μL, 4.77 mmol), DIAD (750 μL, 3.82 mmol) and triphenylphosphine (1.00 g, 3.82 mmol) in THF (6.4 mL). The crude product was purified by flash column chromatography eluting petroleum ether (100%) to afford the title compound as a colourless oil (569 mg, 58%).

¹H NMR (500 MHz, CDC₃) δ 7.88-7.81 (2H, m), 7.23-7.15 (1H, m), 4.14 (2H, t, J=6.6 Hz), 1.92-1.82 (1H, m), 1.80-1.73 (2H, m), 0.99 (6H, d, J=6.6 Hz).

### 1-(2-(Isopentyloxy)-4-nitrophenyl)-4-methylpiperazine

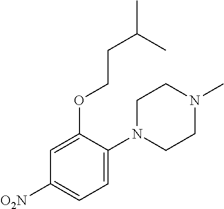 [SMILES: CC(C)CCOc1cc([N+](=O)[O-])ccc1N1CCN(C)CC1]

General procedure 11 was applied to 1-fluoro-2-(isopentyloxy)-4-nitrobenzene (569 mg, 2.50 mmol), N-methylpiperazine (555 μL, 5.00 mmol), potassium carbonate (415 mg, 3.00 mmol) in acetonitrile (5.0 mL) to afford the title compound as a yellow oil (693 mg, 90%). No further purification was required.

¹H NMR (500 MHz, CDC₃) δ 7.78 (1H, dd, J=8.8 and 2.5 Hz), 7.68 (1H, d, J=2.5 Hz), 6.94 (1H, d, J=8.8 Hz), 4.09 (2H, t, J=6.4 Hz), 3.27-3.23 (4H, m), 2.63-2.58 (4H, m), 2.35 (3H, s), 1.94-1.82 (1H, m), 1.77-1.69 (2H, m), 1.00 (6H, d, J=6.0 Hz).

### 3-(Isopentyloxy)-4-(4-methylpiperazin-1-yl)aniline

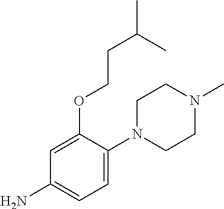 [SMILES: CC(C)CCOc1cc(N)ccc1N1CCN(C)CC1]

General procedure 12 was applied to 3-(isopentyloxy)-4-(4-methylpiperazin-1-yl)aniline (693 mg, 2.26 mmol) and palladium on carbon (69.0 mg, 0.651 mmol) in ethanol (20 mL) was stirred for 18 h at room temperature under a hydrogen atmosphere. The resulting mixture was filtered, washing with ethanol (3×2.0 mL). The filtrate was concentrated under reduced pressure to afford the title compound as a brown oil (630 mg, 100%).

¹H NMR (500 MHz, MeOD) δ 6.64 (1H, d, J=8.4 Hz), 6.30 (1H, d, J=2.4 Hz), 6.17 (1H, dd, J=8.4 and 2.4 Hz), 3.86 (1H, t, J=6.4 Hz), 2.88-2.81 (4H, m), 2.53-2.46 (4H, m), 2.21 (3H, s), 1.83-1.71 (1H, m), 1.64-1.54 (2H, m), 0.89 (6H, d, J=6.2 Hz).

### 2-((3-(Isopentyloxy)-4-(4-methylpiperazin-1-yl)phenyl)amino)-8-methyl-5-((triisopropylsilyl)ethynyl)pyrido[2,3-d]pyrimidin-7(8H)-one

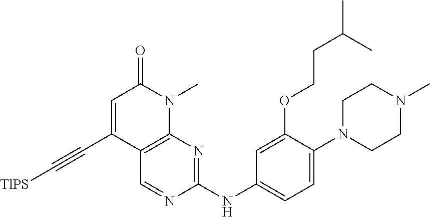

General procedure 13 was applied to 3-(isopentyloxy)-4-(4-methylpiperazin-1-yl)aniline (432 mg, 1.55 mmol) with 8-methyl-2-(methylsulfonyl)-5-((triisopropylsilyl)ethynyl)pyrido[2,3-d]pyrimidin-7(8H)-one (264 mg, 0.631 mmol) and trifluoroacetic acid (238 μL, 1.55 mmol) in acetonitrile (6.3 mL). The crude material was purified by flash column chromatography eluting with methanol (10%) in dichloromethane to yield the title compound as a yellow solid (316 mg, 81%).

¹H NMR (500 MHz, CDCl₃) δ 8.84 (1H, s), 7.89-7.85 (1H, m), 7.47-7.43 (1H, m), 7.06 (1H, d, J=8.6 Hz), 6.91-6.86 (1H, m), 6.62 (1H, s), 4.04 (2H, t, J=6.4 Hz), 3.70-3.60 (5H, m), 3.50-3.44 (2H, m), 3.27-3.18 (2H, m), 3.08-3.02 (2H, m), 2.86 (3H, s), 1.82-1.69 (3H, m), 1.23-1.07 (21H, m), 0.97 (6H, d, J=6.4 Hz).

### 5-Ethynyl-2-((3-(isopentyloxy)-4-(4-methylpiperazin-1-yl)phenyl)amino)-8-methylpyrido[2,3-d]pyrimidin-7(8H)-one

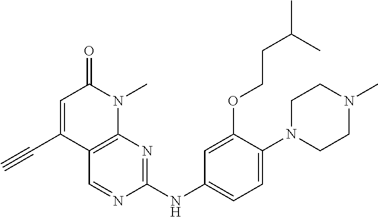 [SMILES: C#Cc1cc(=O)n(C)c2nc(Nc3ccc(N4CCN(C)CC4)c(OCCC(C)C)c3)ncc12]

General procedure 15 was applied to 2-((3-(isopentyloxy)-4-(4-methylpiperazin-1-yl)phenyl)amino)-8-methyl-5-((triisopropylsilyl)ethynyl)pyrido[2,3-d]pyrimidin-7(8H)-one (316 mg, 0.512 mmol) with potassium fluoride (298 mg, 5.12 mmol) in DMF (5.0 mL). The crude material was purified by flash column chromatography eluting with methanol (10%) in dichloromethane to yield the title compound as a yellow solid (103 mg, 44%).

(ES, m/z): [M+1]+=461

¹H NMR (500 MHz, CDCl₃) δ 8.82 (1H, s), 7.60-7.56 (1H, m), 7.41-7.37 (1H, m), 7.08-7.01 (1H, m), 6.91 (1H, d, J=8.5 Hz), 6.63 (1H, s), 4.04 (2H, t, J=6.4 Hz), 3.69 (3H, s), 3.61 (1H, s), 3.12-3.08 (4H, m), 2.63-2.59 (4H, m), 2.36 (3H, s), 1.91-1.79 (1H, m), 1.79-1.72 (2H, m), 0.98 (6H, d, J=6.8 Hz).

### Example 217

### Cyclopentylacetyl chloride

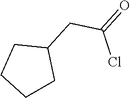

A solution of cyclopentaneacetic acid (1.00 g, 7.80 mmol, 1.0 eq.) in dichloromethane (10 mL) was treated with DMF (100 μL, 1.29 mmol, 0.17 eq.) followed by the addition of thionyl chloride (1.02 g, 8.58 mmol, 1.1 eq.) dropwise at 0° C. The resulting mixture was stirred for 3 hours at room temperature. The resulting mixture was concentrated under reduced pressure to yield the title compound as a light yellow oil (1.00 g, 87%).

### 2-Cyclopentyl-N-(2-fluoro-5-nitrophenyl)acetamide

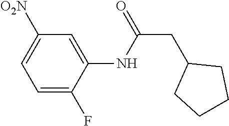 [SMILES: O=C(CC1CCCC1)Nc1cc([N+](=O)[O-])ccc1F]

A solution of 2-fluoro-5-nitroaniline (900 mg, 5.76 mmol, 1.0 eq.) in dichloromethane (10 mL) was treated with cyclopentylacetyl chloride (1.01 g, 6.92 mmol, 1.2 eq.) followed by the addition of triethylamine (1.60 mL, 11.5 mmol, 2.0 eq.) dropwise at room temperature. The resulting mixture was stirred for 14 hours at room temperature. The resulting mixture was extracted with dichloromethane (3×50 mL). The combined organic layers were washed with sodium hydrogen carbonate (3×50 mL), dried (Na₂SO₄) and concentrated under reduced pressure to yield the title compound as a yellow solid (1.30 g, 84%).

### 2-Cyclopentyl-N-[2-(4-methylpiperazin-1-yl)-5-nitrophenyl]acetamide

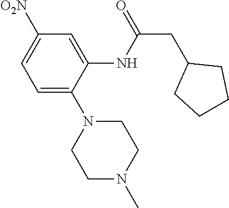 [SMILES: CN1CCN(c2ccc([N+](=O)[O-])cc2NC(=O)CC2CCCC2)CC1]

A solution of 2-cyclopentyl-N-(2-fluoro-5-nitrophenyl)acetamide (1.10 g, 4.13 mmol, 1.0 eq.) in 1-methylpiperazine (5.0 mL) was stirred for 14 hours at 100° C. The residue was purified by flash column chromatography eluting with ethyl acetate (20%) in 40-60 petroleum ether to afford as a yellow solid (370 mg, 26%).

### N-[5-Amino-2-(4-methylpiperazin-1-yl)phenyl]-2-cyclopentylacetamide

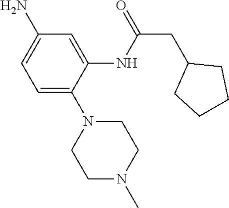 [SMILES: CN1CCN(c2ccc(N)cc2NC(=O)CC2CCCC2)CC1]

A solution of 2-cyclopentyl-N-[2-(4-methylpiperazin-1-yl)-5-nitrophenyl]acetamide (300 mg, 0.866 mmol, 1.0 eq.) in dioxane (10 mL) and water (4.0 mL) was treated with ammonium chloride (463 mg, 8.66 mmol, 10 eq.) followed by iron (483 mg, 8.66 mmol, 10 eq.). The resulting mixture was stirred for 2 hours at 60° C. The resulting mixture was concentrated under reduced pressure then diluted with dichloromethane (20 mL). The resulting mixture was filtered, the filter cake was washed with dichloromethane (3×10 mL). The filtrate was concentrated under reduced pressure to yield the title compound as a yellow oil (300 mg, 98%).

### 2-Cyclopentyl-N-[5-((8-methyl-7-oxo-5-[2-(triisopropylsilyl)ethynyl]pyrido[2,3-d]pyrimidin-2-ylamino)-2-(4-methylpiperazin-1-yl)phenyl]acetamide

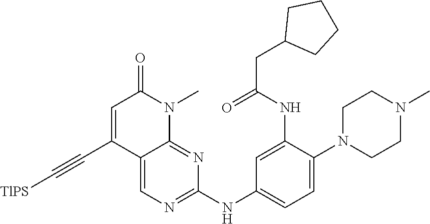

General procedure 13 was applied to 2-methanesulfonyl-8-methyl-5-[2-(triisopropylsilyl)ethynyl]pyrido[2,3-d]pyrimidin-7-one (270 mg, 0.643 mmol), N-[5-amino-2-(4-methylpiperazin-1-yl)phenyl]-2-cyclopentylacetamide (244 mg, 0.772 mmol) and trifluoroacetic acid (45.5 mg, 0.398 mmol) in 2-butanol (5.0 mL), stirred for 14 hours at 100° C. The residue was purified by reverse-phase flash column chromatography eluting with acetonitrile (50-100%) in water (0.1% trifluoroacetic acid) to yield the title compound as a yellow solid (100 mg, 23%).

### 2-Cyclopentyl-N-(5-((5-ethynyl-8-methyl-7-oxo-7,8-dihydropyrido[2,3-d]pyrimidin-2-yl)amino)-2-(4-methylpiperazin-1-yl)phenyl)acetamide

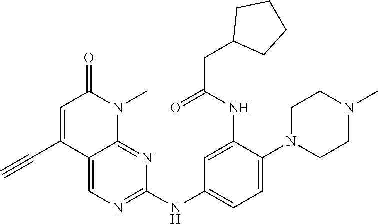 [SMILES: C#Cc1cc(=O)n(C)c2nc(Nc3ccc(N4CCN(C)CC4)c(NC(=O)CC4CCCC4)c3)ncc12]

General procedure 15 was applied to 2-cyclopentyl-N-[5-((8-methyl-7-oxo-5-[2-(triisopropylsilyl)ethynyl]pyrido[2,3-d]pyrimidin-2-ylamino)-2-(4-methylpiperazin-1-yl)phenyl]acetamide (90.0 mg, 0.150 mmol) and potassium fluoride (79.7 mg, 1.37 mmol) in THF (2.0 mL), DMF (2.0 mL) and water (90 μL), stirred for 2 hours at 60° C. The residue was washed with acetonitrile/water (1/1) (3×5.0 mL). The residue was purified by reverse-phase flash column chromatography eluting with acetonitrile (10-50%) in water (0.1% trifluoroacetic acid) to yield the title compound as a yellow solid (31.2 mg, 46%).

(ES, m/z): [M+H]⁺=500.15

¹H-NMR (400 MHz, DMSO-d₆) δ 10.20 (s, 1H), 8.79 (d, J=8.7 Hz, 2H), 8.63 (s, 1H), 7.39 (dd, J=8.6, 2.6 Hz, 1H), 7.16 (d, J=8.7 Hz, 1H), 6.57 (s, 1H), 5.04 (s, 1H), 3.58 (s, 3H), 2.80 (t, J=4.7 Hz, 4H), 2.54 (s, 4H), 2.41 (d, J=7.4 Hz, 2H), 2.29-2.19 (m, 4H), 1.79 (dq, J=11.8, 6.4 Hz, 2H), 1.70-1.59 (m, J=4.6 Hz, 2H), 1.53 (tt, J=7.5, 3.5 Hz, 2H), 1.21 (dq, J=11.9, 7.6 Hz, 2H).

### Example 218

### N-(Cyclopentylmethyl)-2-fluoro-5-nitrobenzenesulfonamide

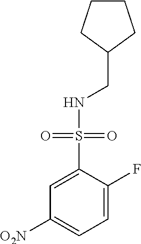 [SMILES: O=[N+]([O-])c1ccc(F)c(S(=O)(=O)NCC2CCCC2)c1]

To a stirred solution of 2-fluoro-5-nitrobenzenesulfonyl chloride (3.00 g, 12.5 mmol, 1.0 eq.) and triethylamine (1.27 g, 12.5 mmol, 1.0 eq.) in dichloromethane (30 mL) was added 1-cyclopentylmethanamine (1.24 g, 12.5 mmol, 1.0 eq.) in portions at room temperature under nitrogen atmosphere. The resulting mixture was stirred for 1 hour at room temperature under nitrogen atmosphere. The residue was purified by flash column chromatography eluting with ethyl acetate (12%) in 40-60 petroleum ether to afford the title compound as a light yellow solid (2.50 g, 66%).

### N-(Cyclopentylmethyl)-2-(4-methylpiperazin-1-yl)-5-nitrobenzenesulfonamide

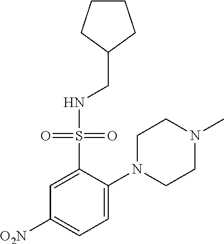 [SMILES: CN1CCN(c2ccc([N+](=O)[O-])cc2S(=O)(=O)NCC2CCCC2)CC1]

General procedure 11 was applied to N-(cyclopentylmethyl)-2-fluoro-5-nitrobenzenesulfonamide (2.50 g, 8.27 mmol), potassium carbonate (1.14 g, 8.27 mmol) and 1-methylpiperazine (1.66 g, 16.5 mmol) in acetonitrile (25 mL), stirred overnight at 80° C. The residue was purified by flash column chromatography eluting with ethyl acetate (25%) in 40-60 petroleum ether to afford the title compound as a yellow solid (2.20 g, 69%).

### 5-Amino-N-(cyclopentylmethyl)-2-(4-methylpiperazin-1-yl)benzenesulfonamide

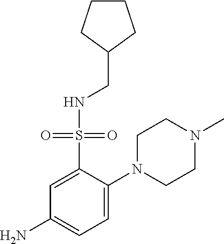 [SMILES: CN1CCN(c2ccc(N)cc2S(=O)(=O)NCC2CCCC2)CC1]

General procedure 12 was applied to N-(cyclopentylmethyl)-2-(4-methylpiperazin-1-yl)-5-nitrobenzenesulfonamide (800 mg, 2.09 mmol) and palladium on carbon (160 mg, 1.50 mmol) in THF (24 mL) to yield the title compound as a brown solid (650 mg, 88%).

### N-(Cyclopentylmethyl)-5-((8-methyl-7-oxo-5-[2-(triisopropylsilyl)ethynyl]pyrido[2,3-d]p yrimidin-2-ylamino)-2-(4-methylpiperazin-1-yl)benzenesulfonamide

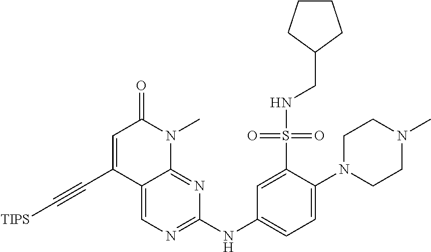

General procedure 13 was applied to 5-amino-N-(cyclopentylmethyl)-2-(4-methylpiperazin-1-yl)benzenesulfonamide (400 mg, 1.13 mmol), 2-methanesulfonyl-8-methyl-5-[2-(triisopropylsilyl)ethynyl]pyrido[2,3-d]pyrimidin-7-one (714 mg, 1.70 mmol) in butan-2-ol (4.0 mL), stirred for 3 hours at 100° C. The residue was purified by flash column chromatography eluting with methanol (12%) in dichloromethane to afford the title compound as a yellow solid (300 mg, 38%).

### N-(Cyclopentylmethyl)-5-((5-ethynyl-8-methyl-7-oxopyrido[2,3-d]pyrimidin-2-ylamino)-2-(4-methylpiperazin-1-yl)benzenesulfonamide

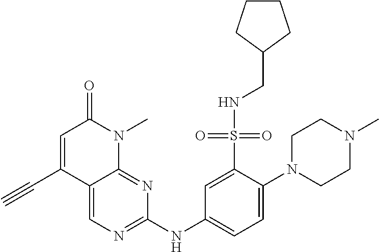 [SMILES: C#Cc1cc(=O)n(C)c2nc(Nc3ccc(N4CCN(C)CC4)c(S(=O)(=O)NCC4CCCC4)c3)ncc12]

General procedure 15 was applied to N-(cyclopentylmethyl)-5-((8-methyl-7-oxo-5-[2-(triisopropylsilyl)ethynyl]pyrido[2,3-d]pyrimidin-2-ylamino)-2-(4-methylpiperazin-1-yl)benzenesulfonamide (150 mg, 0.217 mmol) and potassium fluoride (126 mg, 2.17 mmol) in THF (2.0 mL) and DMF (2.0 mL), stirred for 1 hour at room temperature. The crude product was purified by Prep-HPLC eluting with acetonitrile (23-41%) in water (0.1% formic acid), to afford the title compound as a yellow solid (20.0 mg, 16%).

(ES, m/z): [M+H]⁺=536.25

¹H-NMR (400 MHz, DMSO-d₆) δ 10.54 (s, 1H), 8.83 (s, 1H), 8.65 (s, 1H), 7.89 (dd, J=8.8, 2.6 Hz, 1H), 7.58 (d, J=8.7 Hz, 1H), 6.64 (s, 1H), 6.54 (t, J=6.3 Hz, 1H), 5.07 (s, 1H), 3.60 (s, 3H), 2.94 (t, J=4.6 Hz, 4H), 2.70 (d, J=6.8 Hz, 2H), 2.25 (s, 3H), 2.55 (t, J=4.6 Hz, 4H), 1.94 (p, J=7.4 Hz, 1H), 1.72-1.57 (m, 2H), 1.48 (q, J=8.0, 7.6 Hz, 4H), 1.13 (t, J=6.8 Hz, 2H).

### Example 219

### 3-Cyclopentyl-1-(2-fluoro-5-nitrophenyl)urea

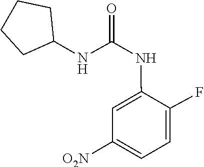 [SMILES: O=C(Nc1cc([N+](=O)[O-])ccc1F)NC1CCCC1]

A solution of 2-fluoro-5-nitroaniline (5.50 g, 35.2 mmol, 1.0 eq.), CDI (11.43 g, 70.4 mmol, 2.0 eq.), triethylamine (7.13 g, 70.46 mmol, 2 eq.) and dichloromethane (60 mL) was stirred for 2 hours at room temperature. To the above mixture was added cyclopentanamine (6.00 g, 70.4 mmol, 2.0 eq.) dropwise at room temperature. The resulting mixture was stirred for an additional 3 hours at room temperature. The residue was purified by reverse-phase flash column chromatography eluting with acetonitrile (40-90%) in water (0.1% trifluoroacetic acid) to afford the title compound as a yellow solid (3.00 g, 32%).

### 3-Cyclopentyl-1-[2-(4-methylpiperazin-1-yl)-5-nitrophenyl]urea

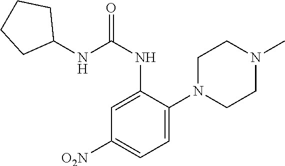 [SMILES: CN1CCN(c2ccc([N+](=O)[O-])cc2NC(=O)NC2CCCC2)CC1]

General procedure 11 was applied to 3-cyclopentyl-1-(2-fluoro-5-nitrophenyl)urea (3.10 g, 11.6 mmol), 1-methylpiperazine (1.39 g, 13.9 mmol) and potassium carbonate (3.21 g, 23.2 mmol) and DMF (30 mL). The residue was purified by reverse-phase flash column chromatography eluting with acetonitrile (40-90%) in water (0.1% trifluoroacetic acid) to afford the title compound as a red solid (3.00 g, 74%).

### 1-[5-Amino-2-(4-methylpiperazin-1-yl)phenyl]-3-cyclopentylurea

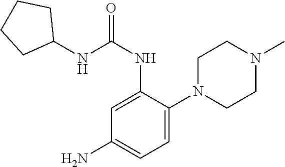 [SMILES: CN1CCN(c2ccc(N)cc2NC(=O)NC2CCCC2)CC1]

General procedure 12 was applied to 3-cyclopentyl-1-[2-(4-methylpiperazin-1-yl)-5-nitrophenyl]urea (1.50 g, 4.32 mmol) and palladium on carbon (150 mg) in methanol (60%). The residue was purified by reverse-phase flash column chromatography eluting with acetonitrile (50-20%) in water (0.1% trifluoroacetic acid) to afford the title compound as a white solid (1.20 g, 87%).

### 3-Cyclopentyl-1-[5-({8-methyl-7-oxo-5-[2-(triisopropylsilyl)ethynyl]pyrido[2,3-d]pyrimidin-2-yl}amino)-2-(4-methylpiperazin-1-yl)phenyl]urea

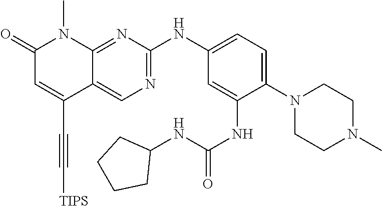

General procedure 13 was applied to 1-[5-amino-2-(4-methylpiperazin-1-yl)phenyl]-3-cyclopentylurea (200 mg, 0.630 mmol), 2-methanesulfonyl-8-methyl-5-[2-(triisopropylsilyl)ethynyl]pyrido[2,3-d]pyrimidin-7-one (714 mg, 1.70 mmol) and trifluoroacetic acid (215 mg, 1.89 mmol) and butan-2-ol (4 mL), stirred overnight at 110° C. The residue was purified by reverse-phase flash column chromatography eluting with acetonitrile (50-90%) in water (0.1% trifluoroacetic acid) to afford the title compound as a yellow solid (80.0 mg, 19%).

### 3-Cyclopentyl-1-[5-({5-ethynyl-8-methyl-7-oxopyrido[2,3-d]pyrimidin-2-yl}amino)-2-(4-methylpiperazin-1-yl)phenyl]urea

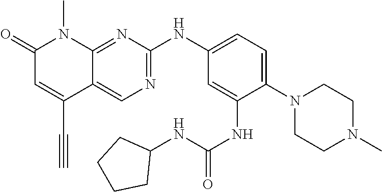 [SMILES: C#Cc1cc(=O)n(C)c2nc(Nc3ccc(N4CCN(C)CC4)c(NC(=O)NC4CCCC4)c3)ncc12]

General procedure 15 was applied to 3-cyclopentyl-1-[5-({8-methyl-7-oxo-5-[2-(triisopropylsilyl)ethynyl]pyrido[2,3-d]pyrimidin-2-yl}amino)-2-(4-methylpiperazin-1-yl)phenyl]urea (150 mg, 0.228 mmol) and potassium fluoride (132 mg, 2.28 mmol) in DMF (1.5 mL), THF (1.5 mL) and water (0.30 mL), stirred for 1 hour at 60° C. The crude product was purified by Prep-HPLC eluting with acetonitrile (10-50%) in water (0.1% trifluoroacetic acid) to afford the title compound as a yellow solid (40.0 mg, 34%).

(ES, m/z): [M+H]⁺=501.30

¹H-NMR (400 MHz, DMSO-d₆) δ 10.10 (s, 1H), 8.76 (s, 1H), 8.68 (s, 1H), 7.65 (s, 1H), 7.25-7.04 (m, 3H), 6.56 (s, 1H), 5.03 (s, 1H), 3.97-3.87 (m, 1H), 3.60 (s, 3H), 2.78-2.72 (m, 3H), 2.56 (s, 4H), 2.27 (s, 3H), 1.87 (d, J=8.8 Hz, 2H), 1.67 (s, 2H), 1.55 (s, 2H), 1.42 (d, J=16.2 Hz, 2H), 1.26-1.22 (m, 1H).

### Example 220

### 5-(2-(2-Fluoro-5-nitrophenoxy)ethyl)-1-methyl-1H-pyrazole

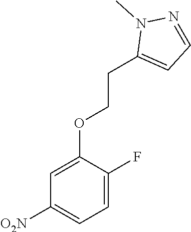 [SMILES: Cn1nccc1CCOc1cc([N+](=O)[O-])ccc1F]

General procedure 22 was applied to 2-fluoro-5-nitrophenol (500 mg, 3.18 mmol), 2-(1-methyl-1H-pyrazole-3-yl)ethan-1-ol (482 μL, 3.82 mmol), DIAD (750 μL, 3.82 mmol) and triphenylphosphine (1.00 g, 3.82 mmol) in THF (6.4 mL). The crude product was purified by flash column chromatography eluting with ethyl acetate (50%) in petroleum ether to afford the title compound as a colourless oil (952 mg, quant).

¹H NMR (500 MHz, CDC₃) 67.91-7.83 (2H, m), 7.31 (1H, d, J=2.3 Hz), 7.26-7.17 (1H, m), 6.19 (1H, d, J=2.3 Hz), 4.40 (2H, t, J=7.0 Hz), 3.22 (2H, t, J 7.0 Hz).

### 1-Methyl-4-(2-(2-(1-methyl-1H-pyrazol-5-yl)ethoxy)-4-nitrophenyl)piperazine

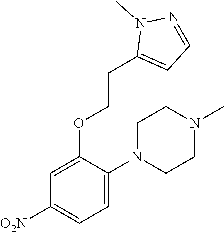 [SMILES: CN1CCN(c2ccc([N+](=O)[O-])cc2OCCc2ccnn2C)CC1]

General procedure 11 was applied to 5-(2-(2-fluoro-5-nitrophenoxy)ethyl)-1-methyl-1H-pyrazole (952 mg, 3.59 mmol), N-methylpiperazine (600 μL, 5.39 mmol) and potassium carbonate (595 mg, 4.31 mmol) in acetonitrile (7.2 mL) to afford the title compound as a yellow oil (1.06 g, 77%). No further purification was required.

¹H NMR (500 MHz, CDCl₃) δ 7.86 (1H, dd, J=8.8 and 2.5 Hz), 7.74 (1H, d, J=2.5 Hz), 7.31 (1H, d, J=2.1 Hz), 6.87 (1H, d, J=8.8 Hz), 6.17 (1H, d, J=2.1 Hz), 4.38 (1H, t, J=6.7 Hz), 3.89 (3H, s), 3.26-3.18 (6H, m), 2.56 (4H, m), 2.37 (3H, s).

### 3-(2-(1-Methyl-1H-pyrazol-5-yl)ethoxy)-4-(4-methylpiperazin-1-yl)aniline

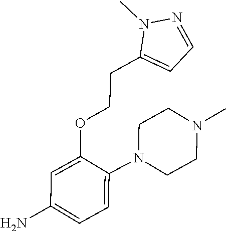 [SMILES: CN1CCN(c2ccc(N)cc2OCCc2ccnn2C)CC1]

General procedure 12 was applied to 1-methyl-4-(2-(2-(1-methyl-1H-pyrazol-5-yl)ethoxy)-4-nitrophenyl)piperazine (1.06 g, 3.07 mmol) and palladium on carbon (106 mg, 1.00 mmol) in ethanol (20 mL), stirred for 18 hours at room temperature under a hydrogen atmosphere. The resulting mixture was filtered, washing with ethanol (3×2.0 mL). The filtrate was concentrated under reduced pressure to afford the title compound as a brown oil (859 mg, 89%).

### 8-Methyl-2-((3-(2-(1-methyl-1H-pyrazol-5-yl)ethoxy)-4-(4-methylpiperazin-1-yl)phenyl)amino)-5-((triisopropylsilyl)ethynyl)pyrido[2,3-d]pyrimidin-7(8H)-one

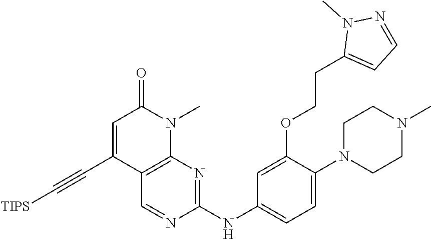

General procedure 13 was applied to 3-(2-(1-methyl-1H-pyrazol-5-yl)ethoxy)-4-(4-methylpiperazin-1-yl)aniline (439 mg, 1.39 mmol) with 8-methyl-2-(methylsulfonyl)-5-((triisopropylsilyl)ethynyl)pyrido[2,3-d]pyrimidin-7(8H)-one (429 mg, 1.02 mmol) and trifluoroacetic acid (111 μL, 1.39 mmol) in acetonitrile (10.2 mL). The crude material was purified by flash column chromatography eluting with methanol (10%) in dichloromethane to yield the title compound as a yellow solid (314 mg, 47%).

¹H NMR (500 MHz, CDCl₃) δ 8.88 (1H, s), 7.54-7.50 (1H, m), 7.41-7.37 (1H, m), 7.32 (1H, d, J=2.2 Hz), 7.10-7.06 (1H, m), 6.91 (1H, d, J=8.6 Hz), 6.65 (1H, s), 6.12 (1H, d, J=2.2 Hz), 4.37 (1H, t, J=6.6 Hz), 3.89 (3H, s), 3.70 (3H, s), 3.59-3.53 (2H, m), 3.45-3.38 (2H, m), 3.23-3.13 (4H, m), 3.07-3.02 (2H, m), 2.87 (3H, s).

### 5-Ethynyl-8-methyl-2-((3-(2-(1-methyl-1H-pyrazol-5-yl)ethoxy)-4-(4-methylpiperazin-1-yl)phenyl)amino)pyrido[2,3-d]pyrimidin-7(8H)-one

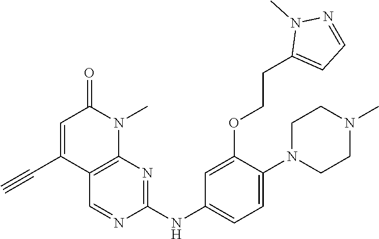 [SMILES: C#Cc1cc(=O)n(C)c2nc(Nc3ccc(N4CCN(C)CC4)c(OCCc4ccnn4C)c3)ncc12]

General procedure 15 was applied to 8-methyl-2-((3-(2-(1-methyl-1H-pyrazol-5-yl)ethoxy)-4-(4-methylpiperazin-1-yl)phenyl)amino)-5-((triisopropylsilyl)ethynyl)pyrido[2,3-d]pyrimidin-7(8H)-one (314 mg, 0.481 mmol) with potassium fluoride (279 mg, 4.81 mmol) in DMF (5.0 mL). The crude material was purified by flash chromatography eluting with methanol (10%) in dichloromethane to yield the title compound as a yellow solid (80.0 mg, 33%).

(ES, m/z): [M+1]+=499

¹H NMR (500 MHz, CDCl₃) δ 8.80 (1H, s), 8.10-8.07 (1H, m), 7.47-7.44 (1H, m), 7.30-7.25 (1H, m), 7.07 (1H, dd, J=8.5 and 2.4 Hz), 6.89 (1H, d, J=8.5 Hz), 6.61 (1H, s), 6.14 (1H, d, J=2.2 Hz), 4.31 (2H, t, J=7.0 Hz), 3.86 (3H, s), 3.65 (1H, s), 3.46 (3H, s), 3.18 (2H, t, J=7.0 Hz), 3.09-3.05 (4H, m), 2.67-2.63 (4H, m), 2.40 (3H, s).

### Example 221

### N-(Cyclopentylmethyl)-2-fluoro-5-nitrobenzamide

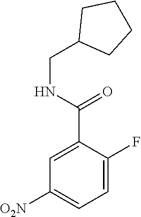 [SMILES: O=C(NCC1CCCC1)c1cc([N+](=O)[O-])ccc1F]

A solution of 2-fluoro-5-nitrobenzoic acid (6.00 g, 32.4 mmol, 1.0 eq.), 1-cyclopentylmethanamine (3.86 g, 38.9 mmol, 1.2 eq.), EDCl (7.46 g, 38.9 mmol, 1.2 eq.), HOBT (4.82 g, 35.6 mmol, 1.1 eq.) and triethylamine (6.56 g, 64.8 mmol, 2.0 eq.) in DMF (50 mL) was stirred overnight at room temperature. The resulting mixture was diluted with water (500 mL). The aqueous layer was extracted with ethyl acetate (2×100 mL). The resulting mixture was concentrated under reduced pressure. The residue was purified by flash column chromatography eluting with ethyl acetate (12%) in 40-60 petroleum ether to afford the title compound as a yellow solid (6.00 g, 69%).

### N-(Cyclopentylmethyl)-2-(4-methylpiperazin-1-yl)-5-nitrobenzamide

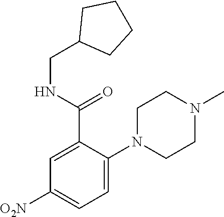 [SMILES: CN1CCN(c2ccc([N+](=O)[O-])cc2C(=O)NCC2CCCC2)CC1]

General procedure 11 was applied to N-(cyclopentylmethyl)-2-fluoro-5-nitrobenzamide (6.00 g, 22.5 mmol), 1-methylpiperazine (2.48 g, 24.8 mmol) and potassium carbonate (6.23 g, 45.1 mmol) in DMF (50 mL) was stirred for 3 hours at 80° C. The resulting mixture was diluted with water (200 mL). The precipitated solids were collected by filtration and washed with water (1×20 mL) to yield the title compound as a yellow solid (5.80 g, 74%).

### 5-Amino-N-(cyclopentylmethyl)-2-(4-methylpiperazin-1-yl)benzamide

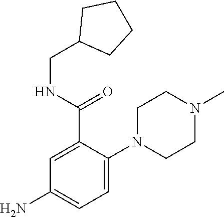 [SMILES: CN1CCN(c2ccc(N)cc2C(=O)NCC2CCCC2)CC1]

General procedure 12 was applied to N-(cyclopentylmethyl)-2-(4-methylpiperazin-1-yl)-5-nitrobenzamide (3.00 g, 8.66 mmol) and palladium on carbon (300 mg) in methanol (50 mL) to yield the title compound as a colourless oil (2.10 g, 76%).

### N-(Cyclopentylmethyl)-5-({8-methyl-7-oxo-5-[2-(triisopropylsilyl)ethynyl]pyrido[2,3-d]pyrimidin-2-yl}amino)-2-(4-methylpiperazin-1-yl)benzamide

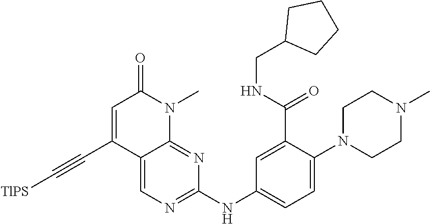

General procedure 13 was applied to 5-amino-N-(cyclopentylmethyl)-2-(4-methylpiperazin-1-yl)benzamide (750 mg, 2.38 mmol), 2-methanesulfonyl-8-methyl-5-[2-(triisopropylsilyl)ethynyl]pyrido[2,3-d]pyrimidin-7-one (1.00 g, 2.38 mmol) and trifluoroacetic acid (540 g, 4.76 mmol) in 2-butanol (10 mL), stirred for 1 hour at 100° C. The residue was purified by reverse-phase flash column chromatography eluting with acetonitrile (10-35%) in water (0.1% formic acid) to yield the title compound as a yellow solid (280 mg, 18%).

### N-(Cyclopentylmethyl)-5-({5-ethynyl-8-methyl-7-oxopyrido[2,3-d]pyrimidin-2-yl}amino)-2-(4-methylpiperazin-1-yl)benzamide

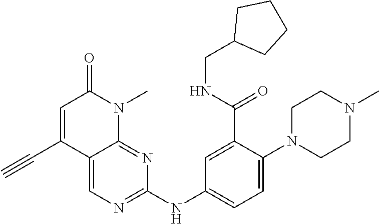 [SMILES: C#Cc1cc(=O)n(C)c2nc(Nc3ccc(N4CCN(C)CC4)c(C(=O)NCC4CCCC4)c3)ncc12]

General procedure 15 was applied to N-(cyclopentylmethyl)-5-({8-methyl-7-oxo-5-[2-(triisopropylsilyl)ethynyl]pyrido[2,3-d]pyrimidin-2-yl}amino)-2-(4-methylpiperazin-1-yl)benzamide (100 mg, 0.152 mmol) and potassium fluoride (44.93 mg, 0.760 mmol) in DMF (2.0 mL), stirred for 1 hour at 60° C. The crude product was purified by Prep-HPLC eluting with acetonitrile (10-45%) in water (0.1% formic acid) to afford the title compound as a yellow solid (6.80 mg, 8%).

(ES, m/z): [M+H]⁺=500.30

¹H-NMR (300 MHz, DMSO-d₆) δ 10.35 (s, 1H), 9.74 (s, 1H), 8.80 (s, 1H), 8.44 (s, 1H), 7.86 (dd, J=8.8, 2.7 Hz, 1H), 7.34 (d, J=8.7 Hz, 1H), 6.60 (s, 1H), 5.06 (s, 1H), 3.60 (s, 3H), 3.26 (s, 2H), 2.94 (d, J=5.5 Hz, 4H), 2.28 (s, 3H), 2.19-2.10 (m, 1H), 1.76 (d, J=6.9 Hz, 2H), 1.67-1.49 (m, 4H), 1.35-1.23 (m, 2H).

### Example 222

### 3-[(2-Fluoro-5-nitrophenyl)carbamoyl]propanoic acid

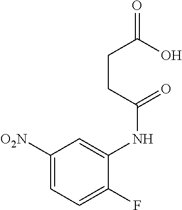 [SMILES: O=C(O)CCC(=O)Nc1cc([N+](=O)[O-])ccc1F]

A solution of 2-fluoro-5-nitroaniline (80.0 g, 512 mmol, 1.0 eq.) in dichloromethane (200 mL) was treated with succinic anhydride (102 g, 1025 mmol, 2.0 eq.) for 5 minutes at room temperature followed by the dropwise addition of triethylamine (51.9 g, 512 mmol, 1.0 eq.). The resulting mixture was stirred for 14 hours at room temperature. The precipitated solids were collected by filtration and washed with dichloromethane (2×30 mL) to yield the title compound as an off-white solid (50.0 g, 38%).

### N-(2-Fluoro-5-nitrophenyl)-4-hydroxybutanamide

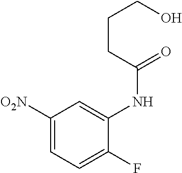 [SMILES: O=C(CCCO)Nc1cc([N+](=O)[O-])ccc1F]

A solution of 3-[(2-fluoro-5-nitrophenyl)carbamoyl]propanoic acid (22.0 g, 85.9 mmol, 1.0 eq.) and N-methylmorpholine (10.4 mL, 94.5 mmol, 1.1 eq.) in THF (1.0 L) at 0° C. was treated with isopropyl chloroformate (11.6 mL, 94.5 mmol, 1.1 eq.) followed by sodium borohydride (7.15 g, 189 mmol, 2.2 eq.) dropwise. The resulting mixture was stirred for 2 hours at 0° C. The reaction was quenched with water at 0° C. then extracted with ethyl acetate (2×200 mL). The combined organic layers were washed with brine (3×100 mL), dried (Na₂SO₄) and concentrated under reduced pressure to yield the title compound as a yellow oil (12.0 g, 58%).

### 4-Hydroxy-N-[2-(4-methylpiperazin-1-yl)-5-nitrophenyl]butanamide

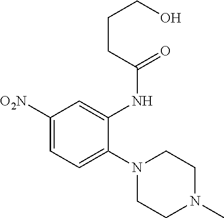 [SMILES: CN1CCN(c2ccc([N+](=O)[O-])cc2NC(=O)CCCO)CC1]

General procedure 11 was applied to N-(2-fluoro-5-nitrophenyl)-4-hydroxybutanamide (5.00 g, 20.6 mmol), N-methylpiperazine, (2.48 g, 24.8 mmol) and potassium carbonate (5.71 g, 41.3 mmol) in acetonitrile (50 mL), stirred for 14 hours at 80° C. The residue was purified by flash column chromatography eluting with methanol (20%) in dichloromethane to afford the title compound as a yellow solid (4.30 g, 64%).

### 4-[(tert-Butyldiphenylsilyl)oxy]-N-[2-(4-methylpiperazin-1-yl)-5-nitrophenyl]butanamide

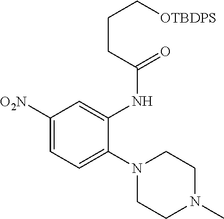

A solution of 4-hydroxy-N-[2-(4-methylpiperazin-1-yl)-5-nitrophenyl]butanamide (3.30 g, 10.2 mmol, 1.0 eq.) in DMF (33 mL) was treated with imidazole (1.39 g, 20.5 mmol, 2.0 eq.) followed by tert-butyldiphenylsilyl chloride (3.38 g, 12.3 mmol, 1.2 eq.). The resulting mixture was stirred for 14 hours at 35° C. The residue was purified by reverse phase flash column chromatography eluting with acetonitrile (50-100%) in water (0.1% trifluoroacetic acid) to yield the title compound as a light yellow solid (3.00 g, 52%).

### N-[5-Amino-2-(4-methylpiperazin-1-yl)phenyl]-4-[(tert-butyldiphenylsilyl)oxy]butanamide

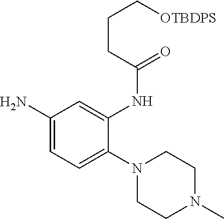

General procedure 12 was applied to 4-[(tert-butyldiphenylsilyl)oxy]-N-[2-(4-methylpiperazin-1-yl)-5-nitrophenyl]butanamide (3.00 g, 5.35 mmol) and palladium on carbon (10%, 300 mg) in methanol (2.0 mL) to yield the title compound as a light yellow solid (2.70 g, 95%).

### 4-[(tert-Butyldiphenylsilyl)oxy]-N-[2-(4-methylpiperazin-1-yl)-5-({7-oxo-5-[2-(triisopropylsilyl)ethynyl]-8H-pyrido[2,3-d]pyrimidin-2-yl}amino)phenyl]butanamide

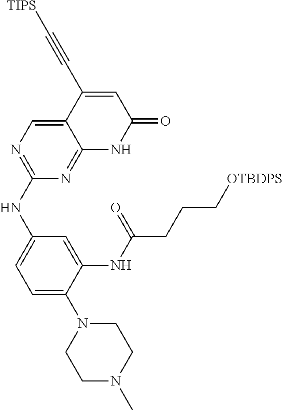

General procedure 13 was applied to 2-methanesulfonyl-5-[2-(triisopropylsilyl)ethynyl]-8H-pyrido[2,3-d]pyrimidin-7-one (2.80 g, 6.90 mmol), N-[5-amino-2-(4-methylpiperazin-1-yl)phenyl]-4-[(tert-butyldiphenylsilyl)oxy]butanamide (4.40 g, 8.28 mmol) and trifluoroacetic acid (1.57 g, 13.8 mmol) in 2-methyl-2-butanol (28 mL), stirred for 12 hours at 100° C. The precipitated solids were collected by filtration and washed with ethyl acetate (2×5.0 mL) to yield the title compound as a yellow solid (1.80 g, 30%).

### 4-Hydroxy-N-[2-(4-methylpiperazin-1-yl)-5-((7-oxo-5-[2-(triisopropylsilyl)ethynyl]-8H-pyrido[2,3-d]pyrimidin-2-ylamino)phenyl]butanamide

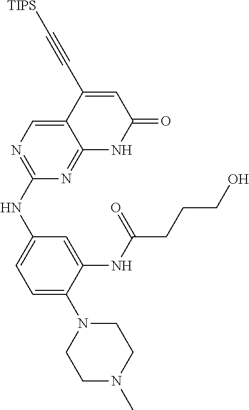

A solution of 4-[(tert-butyldiphenylsilyl)oxy]-N-[2-(4-methylpiperazin-1-yl)-5-((7-oxo-5-[2-(triisopropylsilyl)ethynyl]-8H-pyrido[2,3-d]pyrimidin-2-ylamino)phenyl]butanamide (1.00 g, 1.17 mmol, 1.0 eq.) in 1M hydrochloric acid (10 mL, 11.9 mmol, 10 eq.) in THF (10 mL) was stirred for 14 hours at room temperature. The residue was purified by reverse phase flash column chromatography eluting with acetonitrile (10-70%) in water (0.1% trifluoroacetic acid) to yield the title compound as a yellow solid (300 mg, 42%).

### 3⁴-(4-Methylpiperazin-1-yl)-1⁵-[2-(triisopropylsilyl)ethynyl]-1⁷,1⁸-dihydro-2,4-diaza-1(2,8)-pyrido[2,3-d]pyrimidine-3(1,3)-benzenacyclooctaphane-1⁷,5-dione

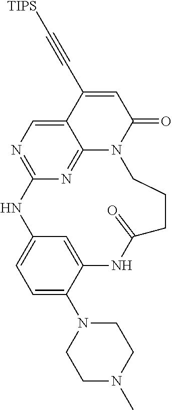

General procedure 20 was applied to 4-hydroxy-N-[2-(4-methylpiperazin-1-yl)-5-((7-oxo-5-[2-(triisopropylsilyl)ethynyl]-8H-pyrido[2,3-d]pyrimidin-2-ylamino)phenyl]butanamide (100 mg, 0.162 mmol), triphenylphosphine (170 mg, 0.648 mmol) and DEAD (113 mg, 0.648 mmol) in THF (1.0 mL). The residue was purified by reverse phase flash column chromatography eluting acetonitrile (10-70%) in water (0.1% trifluoroacetic acid) to yield the title compound as a yellow solid (60.0 mg, 62%).

### 1⁵-ethynyl-3⁴-(4-methylpiperazin-1-yl)-1⁷,1⁸-dihydro-2,4-diaza-1(2,8)-pyrido[2,3-d]pyrimidina-3(1,3)-benzenacyclooctaphane-1⁷,5-dione

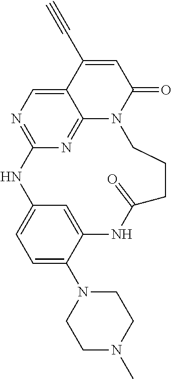 [SMILES: C#Cc1cc(=O)n2c3nc(ncc13)Nc1ccc(N3CCN(C)CC3)c(c1)NC(=O)CCC2]

General procedure 15 was applied to 3⁴-(4-Methylpiperazin-1-yl)-1⁵-[2-(triisopropylsilyl)ethynyl]-1⁷,1⁸-dihydro-2,4-diaza-1(2,8)-pyrido[2,3-d]pyrimidine-3(1,3)-benzenacyclooctaphane-1⁷,5-dione (60.0 mg, 0.100 mmol) and potassium fluoride (58.1 mg, 1.00 mmol) in THF (2.5 mL), DMF (2.5 mL) and water (60 μL), stirred for 2 hours at 60° C. The precipitated solids were collected by filtration and washed with ethyl acetate (2×5.0 mL) to yield the title compound as a yellow solid (12.1 mg, 27%).

(ES, m/z) [M+H]⁺=444.00.

¹H-NMR (400 MHz, DMSO-d₆) δ 10.20 (s, 1H), 8.76 (s, 1H), 8.62 (s, 1H), 8.49 (s, 1H), 7.74 (d, J=2.4 Hz, 1H), 7.02 (d, J=8.5 Hz, 1H), 6.96 (dd, J=8.6, 2.4 Hz, 1H), 6.52 (s, 1H), 5.04 (s, 1H), 3.91 (s, 2H), 2.85 (d, J=5.5 Hz, 4H), 2.46 (s, 4H), 2.31-2.25 (m, 2H), 2.22 (s, 3H), 2.02 (d, J=8.6 Hz, 2H).

### Example 223

### 3-[(tert-Butyldiphenylsilyl)oxy]-N-[(5-fluoro-2-nitrophenyl)methyl]-N-methylpropanamide

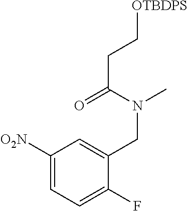

To a stirred solution of 3-[(tert-butyldiphenylsilyl)oxy]-N-[(5-fluoro-2-nitrophenyl)methyl]propanamide (4.00 g, 8.32 mmol, 1.0 eq.) in THF (40 mL) at 0° C. was added sodium hydride (300 mg, 12.5 mmol, 1.5 eq.). The resulting mixture was stirred for 5-10 minutes at 0° C. then methyl iodide (1.08 mL, 25.0 mmol, 3.0 eq.) was added. The resulting mixture was stirred for 30 minutes at 0° C. then quenched with water/ice. The resulting mixture was extracted with ethyl acetate (3×100 mL). The combined organic layers were washed with brine (2×100 mL), dried (Na₂SO₄) and concentrated under reduced pressure, to afford the crude title compound as a brown oil (4.40 g, crude).

### 3-[(tert-butyldiphenylsilyl)oxy]-N-methyl-N-([2-(4-methylpiperazin-1-yl)-5-nitrophenyl]methylpropanamide

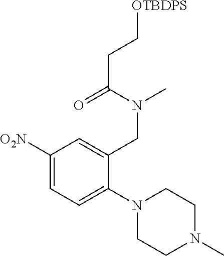

A solution of 3-[(tert-butyldiphenylsilyl)oxy]-N-[(2-fluoro-5-nitrophenyl)methyl]-N-methylpropanamide (4.40 g, 8.89 mmol, 1.0 eq.), N-methylpiperazine (1.07 g, 10.7 mmol, 1.2 eq.), sodium carbonate (1.89 g, 17.8 mmol, 2.0 eq.) in acetonitrile (44 mL) was stirred overnight at 80° C. The resulting mixture was extracted with ethyl acetate (3×100 mL). The combined organic layers were washed with brine (300 mL), dried (Na₂SO₄) and concentrated under reduced pressure. The residue was purified by flash column chromatography eluting with ethyl acetate to afford the title compound as a brown solid (2.27 g, 44%).

### N-([5-Amino-2-(4-methylpiperazin-1-yl)phenyl]methyl-3-[(tert-butyldiphenylsilyl)oxy]-N-methylpropanamide

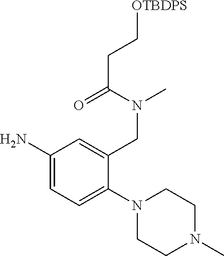

General procedure 12 was applied to 3-[(tert-butyldiphenylsilyl)oxy]-N-methyl-N-([2-(4-methylpiperazin-1-yl)-5-nitrophenyl]methylpropanamide (2.00 g, 3.48 mmol) and palladium on carbon (10%, 400 mg, 3.76 mmol) in THF (50 mL) to yield the crude title compound as a brown yellow solid (1.74 g, 92%).

### 3-[(tert-Butyldiphenylsilyl)oxy]-N-methyl-N-([2-(4-methylpiperazin-1-yl)-5-((7-oxo-5-[2-(triisopropylsilyl)ethynyl]-8H-pyrido[2,3-d]pyrimidin-2-ylamino)phenyl]methylpropanamide

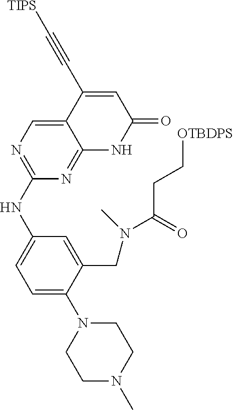

General procedure 13 was applied to N-([5-amino-2-(4-methylpiperazin-1-yl)phenyl]methyl-3-[(tert-butyldiphenylsilyl)oxy]-N-methylpropanamide (2.00 g, 3.67 mmol), 2-methanesulfonyl-5-[2-(triisopropylsilyl)ethynyl]-8H-pyrido[2,3-d]pyrimidin-7-one (1.79 g, 4.40 mmol) and trifluoroacetic acid (545 μL, 7.34 mmol) in 2-butanol (20 mL) stirred overnight at 100° C. The residue was purified by flash column chromatography eluting with methanol (8%) in dichloromethane to afford the title compound as a brown solid (2.15 g, 67%).

### 3-Hydroxy-N-methyl-N-([2-(4-methylpiperazin-1-yl)-5-((7-oxo-5-[2-(triisopropylsilyl)ethynyl]-8H-pyrido[2,3-d]pyrimidin-2-ylamino)phenyl]methylpropanamide

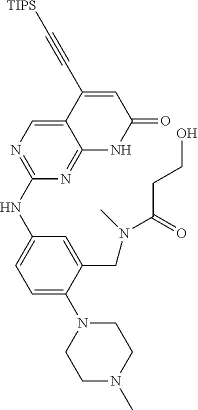

A solution of 3-[(tert-butyldiphenylsilyl)oxy]-N-methyl-N-([2-(4-methylpiperazin-1-yl)-5-((7-oxo-5-[2-(triisopropylsilyl)ethynyl]-8H-pyrido[2,3-d]pyrimidin-2-ylamino)phenyl]methylpropanamide (1.00 g, 1.15 mmol, 1.0 eq.) and hydrogen chloride in methanol (11 mL, 4M) was stirred for 2 hours at 60° C. The resulting mixture was concentrated under reduced pressure. The residue was purified by reverse phase flash column chromatography eluting with acetonitrile (10-40%) in water (0.1% formic acid), to afford the title compound as a brown yellow solid (439 mg, 55%).

### 5-Methyl-3⁴-(4-methylpiperazin-1-yl)-1⁵-((triisopropylsilyl)ethynyl)-1⁷,1⁸-dihydro-2,5-diaza-1(2,8)-pyrido[2,3-d]pyrimidina-3(1,3)-benzenacyclooctaphane-1⁷,6-dione

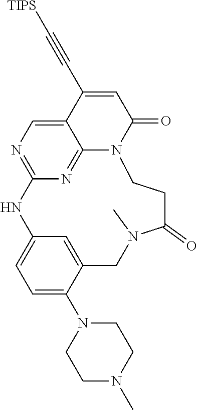

General procedure 20 was applied to 3-hydroxy-N-methyl-N-([2-(4-methylpiperazin-1-yl)-5-((7-oxo-5-[2-(triisopropylsilyl)ethynyl]-8H-pyrido[2,3-d]pyrimidin-2-ylamino)phenyl]methylpropanamide (300 mg, 0.475 mmol), triphenylphosphine (489 mg, 1.90 mmol) and DEAD (298 μL, 1.90 mmol) and THF (3 mL). The residue was purified by flash column chromatography eluting with methanol (8%) in dichloromethane to afford the title compound as a brown yellow solid (220 mg, 75%).

### 1⁵-Ethynyl-5-methyl-3⁴-(4-methylpiperazin-1-yl)-1⁷,1⁸-dihydro-2,5-diaza-1(2,8)-pyrido[2, 3-d]pyrimidina-3(1,3)-benzenacyclooctaphane-1⁷,6-dione

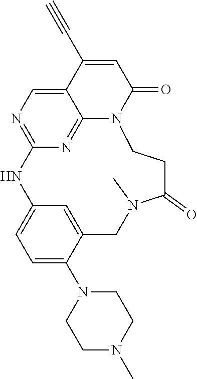 [SMILES: C#Cc1cc(=O)n2c3nc(ncc13)Nc1ccc(N3CCN(C)CC3)c(c1)CN(C)C(=O)CC2]

General procedure 15 was applied to 5-methyl-3⁴-(4-methylpiperazin-1-yl)-15-((triisopropylsilyl)ethynyl)-1⁷,1⁸-dihydro-2,5-diaza-1(2,8)-pyrido[2,3-d]pyrimidina-3(1,3)-benzenacyclooctaphane-1⁷,6-dione (40.0 mg, 65.0 μmol) and potassium fluoride (37.8 mg, 0.650 mmol) in DMF (1.0 mL), THF (1.0 mL) and water (40 μL), stirred for 2 hours at 60° C. The residue was purified by reverse phase flash column chromatography eluting with acetonitrile (10-50%) in water (0.1% ammonium formate), to yield the title compound as a yellow solid (6.70 mg, 22%).

(ES, m/z) [M+H]⁺=458.15

¹H NMR (400 MHz, Chloroform-d) δ 8.83 (s, 1H), 8.44 (s, 1H), 7.32 (s, 1H), 7.03 (s, 1H), 6.85 (d, J=8.4 Hz, 1H), 6.64 (s, 1H), 5.39 (s, 1H), 4.79 (t, J=11.5 Hz, 1H), 4.38 (d, J=111.6 Hz, 2H), 3.90 (d, J=14.8 Hz, 1H), 3.63 (s, 1H), 3.22 (s, 1H), 3.05 (s, 2H), 2.95 (s, 3H), 2.90 (s, 2H), 2.65 (s, 4H), 2.41 (s, 3H).

### Example 224

### tert-Butyl N-(3-[(tert-butyldiphenylsilyl)oxy]propylcarbamate

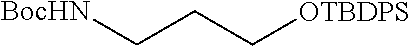

To a stirred solution of tert-butyl N-(3-hydroxypropyl)carbamate (30.0 g, 171 mmol, 1.0 eq.) and tert-butyldiphenylsilyl chloride (70.6 g, 257 mmol, 1.5 eq.) in dichloromethane (300 mL) was added triethylamine (86.6 g, 856 mmol, 5.0 eq.) and DMAP (6.27 g, 51.4 mmol, 0.30 eq.). The resulting mixture was stirred for 16 hours at room temperature then concentrated under reduced pressure. The residue was purified by flash column chromatography eluting with ethyl acetate (8%) in 40-60 petroleum ether to afford the title compound as a colourless oil (45.0 g, 63%).

### (3-Aminopropoxy)(tert-butyl)diphenylsilane

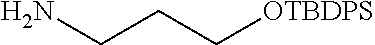

A solution of tert-butyl N-{3-[(tert-butyldiphenylsilyl)oxy]propyl}carbamate (45.0 g, 109 mmol, 1.0 eq.) and trifluoroacetic acid (90 mL) in dichloromethane (450 mL) was stirred for 1 hour at room temperature. The resulting mixture was concentrated under reduced pressure to yield the title compound as a light yellow oil (50.0 g, 73%).

### N-(3-[(tert-Butyldiphenylsilyl)oxy]propyl-2-fluoro-5-nitrobenzamide

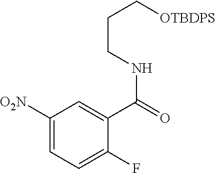

To a stirred solution of (3-aminopropoxy)(tert-butyl)diphenylsilane (30.0 g, 95.7 mmol, 1.0 eq.) and 2-fluoro-5-nitrobenzoic acid (21.3 g, 115 mmol, 1.2 eq.) in dichloromethane (300 mL) was added EDCl (22.0 g, 115 mmol, 1.2 eq.), HOBT (15.5 g, 115 mmol, 1.2 eq.) and triethylamine (29.0 g, 287 mmol, 3.0 eq.). The resulting mixture was stirred for 2 hours at room temperature. The residue was purified by flash column chromatography eluting with ethyl acetate (25%) in 40-60 petroleum ether to afford the title compound as a white solid (7.00 g, 15%).

### N-(3-[(tert-Butyldiphenylsilyl)oxy]propyl-2-(4-methylpiperazin-1-yl)-5-nitrobenzamide

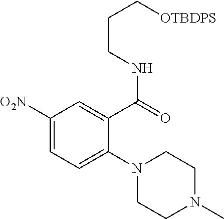

General procedure 11 was applied to N-(3-[(tert-butyldiphenylsilyl)oxy]propyl-2-fluoro-5-nitrobenzamide (3.00 g, 6.24 mmol), N-methylpiperazine (750 mg, 7.49 mmol) and potassium carbonate (1.73 g, 12.5 mmol) in acetonitrile (30 mL). The residue was purified by flash column chromatography eluting with methanol (20%) in dichloromethane to afford the title compound as a yellow solid (2.20 g, 63%).

### 5-Amino-N-{3-[(tert-butyldiphenylsilyl)oxy]propyl}-2-(4-methylpiperazin-1-yl)benzamide

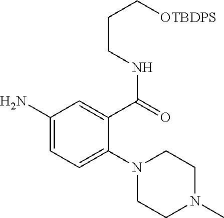

General procedure 12 was applied to N-{3-[(tert-butyldiphenylsilyl)oxy]propyl}-2-(4-methylpiperazin-1-yl)-5-nitrobenzamide (2.20 g, 3.92 mmol) and palladium on carbon (10%, 220 mg) in methanol (20 mL) to yield the title compound as a light yellow solid (1.80 g, 86%).

### N-(3-[(tert-Butyldiphenylsilyl)oxy]propyl-2-(4-methylpiperazin-1-yl)-5-((7-oxo-5-[2-(triisopropylsilyl) ethynyl]-8H-pyrido[2,3-d]pyrimidin-2-ylamino)benzamide

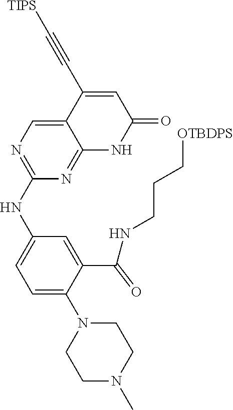

General procedure 13 was applied to 5-amino-N-(3-[(tert-butyldiphenylsilyl)oxy]propyl-2-(4-methylpiperazin-1-yl)benzamide (1.10 g, 2.07 mmol), 2-methanesulfonyl-5-[2-(triisopropylsilyl)ethynyl]-8H-pyrido[2,3-d]pyrimidin-7-one (1.01 g, 2.49 mmol) and trifluoroacetic acid (472 mg, 4.14 mmol) in 2-methyl-2-butanol (11 mL), stirred for 14 hours at 100° C. The precipitated solids were collected by filtration and washed with ethyl acetate (2×5.0 mL) to yield the title compound as a yellow solid (500 mg, 28%).

### N-(3-Hydroxypropyl)-2-(4-methylpiperazin-1-yl)-5-({7-oxo-5-[2-(triisopropylsilyl)ethynyl]-8H-pyrido[2,3-d]pyrimidin-2-yl}amino)benzamide

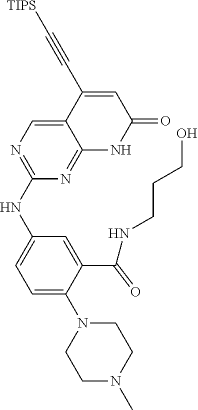

A solution of N-{3-[(tert-butyldiphenylsilyl)oxy]propyl}-2-(4-methylpiperazin-1-yl)-5-({7-oxo-5-[2-(triisopropylsilyl)ethynyl]-8H-pyrido[2,3-d]pyrimidin-2-yl}amino)benzamide (500 mg, 1.17 mmol, 1.0 eq.) in hydrogen chloride in methanol (10.0 mL, 329 mmol, 282 eq.) was stirred for 2 hours at room temperature. The residue was purified by reverse phase flash column chromatography eluting with acetonitrile (10-70%) in water (0.1% trifluoroacetic acid) to yield the title compound as a yellow soldi (300 mg, 69%).

### 3⁴-(4-methylpiperazin-1-yl)-1⁵-((triisopropylsilyl)ethynyl)-1⁷,1⁸-dihydro-2,5-diaza-1(2,8)-pyrido[2,3-d]pyrimidina-3(1,3)-benzenacyclooctaphane-1⁷,4-dione

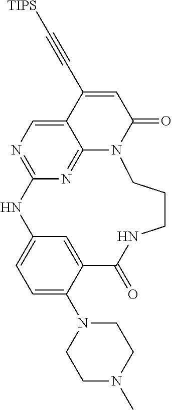

General procedure 20 was applied to N-(3-hydroxypropyl)-2-(4-methylpiperazin-1-yl)-5-((7-oxo-5-[2-(triisopropylsilyl)ethynyl]-8H-pyrido[2,3-d]pyrimidin-2-ylamino)benzamide (200 mg, 0.437 mmol) triphenylphosphine (458 mg, 1.75 mmol) and DIAD (353 mg, 1.75 mmol) in THF (10 mL). The residue was purified by reverse phase flash column chromatography eluting with acetonitrile (50-100%) in water (0.1% formic acid) to yield the title compound as a yellow solid (40.0 mg, 20%).

### 1⁵-ethynyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphane-1⁷,4-dione

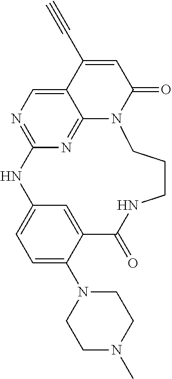 [SMILES: C#Cc1cc(=O)n2c3nc(ncc13)Nc1ccc(N3CCN(C)CC3)c(c1)C(=O)NCCC2]

General procedure 15 was applied to 3⁴-(4-methylpiperazin-1-yl)-15-((triisopropylsilyl)ethynyl)-1⁷,1⁸-dihydro-2,5-diaza-1(2,8)-pyrido[2,3-d]pyrimidina-3(1,3)-benzenacyclooctaphane-1⁷,4-dione (70.0 mg, 0.117 mmol) and potassium fluoride (67.8 mg, 1.17 mmol) in DMF (2.0 mL), THF (2.0 mL) and water (70 μL), stirred for 4 hours at 60° C. The residue was purified by reverse phase flash column chromatography eluting with acetonitrile (10-50%) in water (0.1% trifluoroacetic acid) to yield the title compound as a yellow solid (2.90 mg, 5%).

(ES, m/z) [M+H]⁺=444.20.

¹H-NMR (400 MHz, DMSO-d₆) δ 9.43 (s, 1H), 8.96-8.90 (m, 1H), 8.85 (s, 1H), 7.37-7.20 (m, 2H), 6.70 (s, 1H), 4.86 (s, 1H), 4.34 (s, 2H), 3.52 (s, 4H), 3.25 (s, 4H), 3.08 (s, 2H), 1.72 (s, 2H), 1.21-1.09 (m, 1H).

### Example 225

### 4-[(tert-Butyldiphenylsilyl)oxy]-N-(2-fluoro-5-nitrophenyl)butanamide

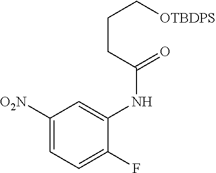

To a stirred solution of N-(2-fluoro-5-nitrophenyl)-4-hydroxybutanamide (11.0 g, 45.4 mmol, 1.0 eq.) in DMF (110 mL) was added imidazole (6.18 g, 90.8 mmol, 2.0 eq.) followed by tert-butyldiphenylsilyl chloride (31.2 g, 113 mmol, 2.5 eq.). The resulting mixture was stirred for 14 hours at room temperature. The residue was purified by reverse phase flash column chromatography eluting with acetonitrile (50-100%0 in water (0.1% trifluoroacetic acid) to yield the title compound as a yellow solid (20.0 g, 91%).

### 4-[(tert-Butyldiphenylsilyl)oxy]-N-(2-fluoro-5-nitrophenyl)-N-methylbutanamide

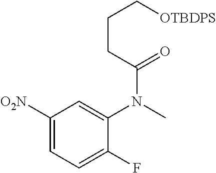

To a stirred solution of 4-[(tert-butyldiphenylsilyl)oxy]-N-(2-fluoro-5-nitrophenyl)butanamide (10.0 g, 20.8 mmol, 1.0 eq.) in THF (2.0 mL) at 0° C. was added sodium hydride (12.0 mg, 0.499 mmol, 1.2 eq.) followed by the addition of methyl iodide (3.54 g, 25.0 mmol, 1.2 eq.). The resulting mixture was stirred for 4 hours at room temperature then quenched with water and extracted with ethyl acetate (3×200 mL). The combined organic layers were washed with brine (2×100 mL), dried (Na₂SO₄) and concentrated under reduced pressure. The residue was purified by flash column chromatography eluting with ethyl acetate (8%) in 40-60 petroleum ether to afford the title compound as a light yellow solid (8.60 g, 83%).

### N-(3-[(tert-Butyldiphenylsilyl)oxy]propyl-N-methyl-2-(4-methylpiperazin-1-yl)-5-nitrobenzamide

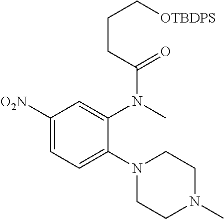

General procedure 11 was applied to 4-[(tert-butyldiphenylsilyl)oxy]-N-(2-fluoro-5-nitrophenyl)-N-methylbutanamide (8.60 g, 3.24 mmol), N-methylpiperazine and potassium carbonate (1.79 g, 12.9 mmol) in acetonitrile (16 mL). The residue was purified by flash column chromatography eluting with methanol (7%) in dichloromethane to afford the title compound as a light yellow oil (8.60 g, 86%).

### N-[5-Amino-2-(4-methylpiperazin-1-yl)phenyl]-4-[(tert-butyldiphenylsilyl)oxy]-N-methyl butanamide

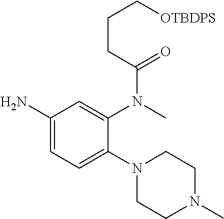

General procedure 12 was applied to 4-[(tert-butyldiphenylsilyl)oxy]-N-methyl-N-[2-(4-methylpiperazin-1-yl)-5-nitrophenyl]butanamide (8.60 g, 14.9 mmol) and palladium on carbon (10%, 2.60 g) in methanol (90 mL) to yield the title compound as a light yellow solid (7.60 g, 93%).

### 4-[(tert-Butyldiphenylsilyl)oxy]-N-methyl-N-[2-(4-methylpiperazin-1-yl)-5-({7-oxo-5-[2-(triisopropylsilyl) ethynyl]-8H-pyrido[2,3-d]pyrimidin-2-yl}amino)phenyl]butanamide

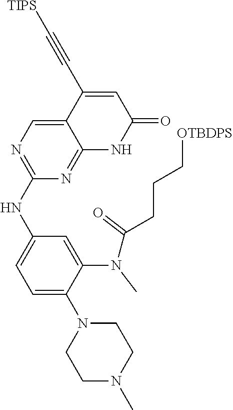

General procedure 13 was applied to N-[5-amino-2-(4-methylpiperazin-1-yl)phenyl]-4-[(tert-butyldiphenylsilyl)oxy]-N-methylbutanamide (4.00 g, 7.34 mmol), 2-methanesulfonyl-5-[2-(triisopropylsilyl)ethynyl]-8H-pyrido[2,3-d]pyrimidin-7-one (3.57 g, 8.81 mmol) and trifluoroacetic acid (1.67 g, 14.7 mmol) in 2-methyl-2-butanol (40 mL), stirred for 24 hours at 80° C. The precipitated solids were collected by filtration and washed with ethyl acetate (2×5.0 mL) to yield the title compound as a yellow solid (3.00 g, 47%).

### 4-Hydroxy-N-methyl-N-[2-(4-methylpiperazin-1-yl)-5-((7-oxo-5-[2-(triisopropyisiyl)ethynyl]-8H-pyrido[2,3-d]pyrimidin-2-ylamino)phenyl]butanamide

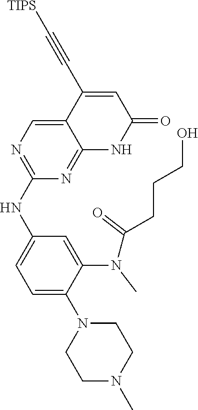

A solution of 4-[(tert-butyldiphenylsilyl)oxy]-N-methyl-N-[2-(4-methylpiperazin-1-yl)-5-((7-oxo-5-[2-(triisopropylsilyl)ethynyl]-8H-pyrido[2,3-d]pyrimidin-2-ylamino)phenyl]butanamide (2.00 g, 2.29 mmol, 1.0 eq.) in 1M hydrogen chloride (10 mL) and THF (10 mL) was stirred for 14 hours at room temperature. The residue was purified by reverse phase flash column chromatography eluting with acetonitrile (10-70%) in water (0.1% trifluoroacetic acid) to yield the title compound as a yellow solid (800 mg, 55%).

### 4-Methyl-3⁴-(4-methylpiperazin-1-yl)-1⁵-((triisopropylsilyl)ethynyl)-1⁷,1⁸-dihydro-2,4-diaza-1(2,8)-pyrido[2,3-d]pyrimidina-3(1,3)-benzenacyclooctaphane-1⁷,5-dione

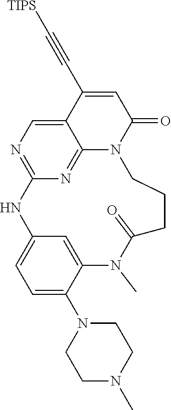

General procedure 20 was applied to 4-hydroxy-N-methyl-N-[2-(4-methylpiperazin-1-yl)-5-((7-oxo-5-[2-(triisopropylsilyl)ethynyl]-8H-pyrido[2,3-d]pyrimidin-2-ylamino)phenyl]butanamide (400 mg, 0.633 mmol), triphenylphosphine (664 mg, 2.53 mmol) and DIAD (366 mg, 1.81 mmol) in THF (4.0 mL). The residue was purified by reverse phase flash column chromatography eluting with acetonitrile (50-100%) in water (0.1% formic acid) to yield the title compound as a yellow solid (150 mg, 38%).

### 1⁵-Ethynyl-4-methyl-3⁴-(4-methylpiperazin-1-yl)-1⁷,1⁸-dihydro-2,4-diaza-1(2,8)-pyrido[2, 3-d]pyrimidina-3(1,3)-benzenacyclooctaphane-1⁷,5-dione

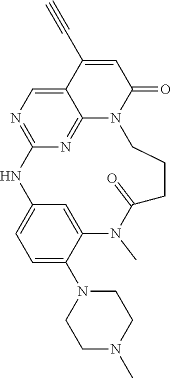 [SMILES: C#Cc1cc(=O)n2c3nc(ncc13)Nc1ccc(N3CCN(C)CC3)c(c1)N(C)C(=O)CCC2]

General procedure 15 was applied to 4-methyl-3⁴-(4-methylpiperazin-1-yl)-15-((triisopropylsilyl)ethynyl)-1⁷,1⁸-dihydro-2,4-diaza-1(2,8)-pyrido[2,3-d]pyrimidina-3(1,3)-benzenacyclooctaphane-1⁷,5-dione (150 mg, 0.244 mmol) and potassium fluoride (142 mg, 2.44 mmol) in THF (2.0 mL), DMF (2.0 mL) and water (150 μL), stirred for 4 hours at room temperature. The residue was purified by reverse phase flash column chromatography eluting with acetonitrile (10-50%) in water (0.1% trifluoroacetic acid) to yield the title compound as a yellow solid (29.5 mg, 26%).

(ES, m/z) [M+H]⁺=458.00.

¹H-NMR (400 MHz, DMSO-d₆) δ 10.18 (s, 1H), 8.74 (s, 1H), 7.56 (s, 1H), 7.09-7.02 (m, 2H), 6.51 (s, 1H), 5.03 (s, 1H), 4.49 (q, J=10.1 Hz, 1H), 3.22 (t, J=11.5 Hz, 1H), 3.10 (s, 3H), 3.05-2.99 (m, 2H), 2.74 (s, 2H), 2.44 (d, J=14.8 Hz, 4H), 2.31 (d, J=12.1 Hz, 1H), 2.22 (s, 4H), 1.93 (t, J=11.4 Hz, 2H).

### Example 226

### 3-((tert-butyldimethylsilyl)oxy)-N-(2-fluoro-5-nitrobenzyl)propan-1-amine

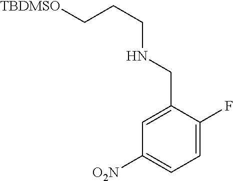 [SMILES: CC(C)(C)[Si](C)(C)OCCCNCc1cc([N+](=O)[O-])ccc1F]

A solution of 2-fluoro-5-nitrobenzaldehyde (1.01 g, 5.97 mmol, 1.0 eq.), 3-((tert-butyldimethylsilyl)oxy)propan-1-amine (1.13 g, 5.97 mmol, 1.0 eq.), sodium acetate (735 mg, 8.96 mmol, 1.5 eq.) and sodium cyanoborohydride (750 mg, 11.9 mmol, 2.0 eq.) in methanol (20 mL) was stirred at room temperature overnight. The reaction mixture was quenched with water (20 mL) and extracted with dichloromethane (3×30 mL). The combined organic layers were dried (MgSO₄) and concentrated under reduced pressure. The crude material was purified by flash column chromatography eluting with ethyl acetate (20-60%) in 40-60 petroleum ether to yield the title compound as a yellow solid (332 mg, 2.80 mmol, 47%).

(ES, m/z) [M+H]⁺=343.3

¹H NMR (500 MHz, CDCl₃) δ 8.34-8.27 (m, 1H), 8.11 (ddd, J=8.9, 4.4, 2.9 Hz, 1H), 7.13 (t, J=8.9 Hz, 1H), 3.86 (s, 2H), 3.74-3.64 (m, 2H), 2.71 (t, J=6.7 Hz, 2H), 1.71 (h, J=6.4 Hz, 2H), 0.82 (d, J=4.1 Hz, 9H), −0.00 (s, 6H).

### 3-((tert-butyldimethylsilyl)oxy)-N-(2-fluoro-5-nitrobenzyl)-N-methylpropan-1-amine

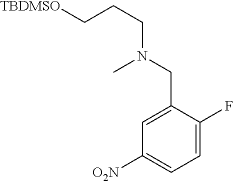 [SMILES: CN(CCCO[Si](C)(C)C(C)(C)C)Cc1cc([N+](=O)[O-])ccc1F]

A solution of 3-((tert-butyldimethylsilyl)oxy)-N-(2-fluoro-5-nitrobenzyl)propan-1-amine (625 mg, 1.83 mmol, 1.0 eq.), paraformaldehyde (247 mg, 8.24 mmol, 4.5 eq.) and sodium cyanoborohydride (132 mg, 2.10 mmol, 1.2 eq.) in methanol (7.5 mL) was refluxed for 4 hours. The reaction mixture was cooled and concentrated under reduced pressure. The crude material was purified by flash column chromatography eluting with ethyl acetate (20-60%) in 40-60 petroleum ether to yield the title compound as a yellow solid (542 mg, 1.51 mmol, 83%).

(ES, m/z) [M+H]⁺=358.3

¹H NMR (500 MHz, CDCl₃) δ 8.33 (dd, J=6.2, 2.9 Hz, 1H), 8.11 (ddd, J=9.0, 4.4, 2.9 Hz, 1H), 7.13 (t, J=8.9 Hz, 1H), 3.64 (t, J=6.3 Hz, 2H), 3.56 (s, 2H), 2.51-2.44 (m, 2H), 2.20 (s, 3H), 1.75-1.66 (m, 2H), 0.83 (s, 9H), −0.00 (s, 6H).

### 3-((tert-butyldimethylsilyl)oxy)-N-methyl-N-(2-(4-methylpiperazin-1-yl)-5-nitrobenzyl)propan-1-amine

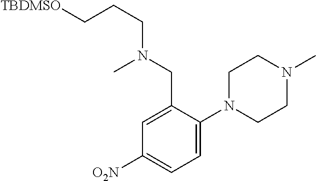 [SMILES: CN1CCN(c2ccc([N+](=O)[O-])cc2CN(C)CCCO[Si](C)(C)C(C)(C)C)CC1]

General procedure 11 was applied to 3-((tert-butyldimethylsilyl)oxy)-N-(2-fluoro-5-nitrobenzyl)-N-methylpropan-1-amine (541 mg, 1.51 mmol), N-methylpiperazine (180 μL, 1.66 mmol) and lithium carbonate (134 mg, 1.81 mmol) in acetonitrile (2.5 mL). The crude material was purified by flash column chromatography eluting with methanol (0-20%) in dichloromethane to yield the title compound as a yellow solid (450 mg, 1.03 mmol, 68%).

(ES, m/z) [M+H]⁺=437.4

¹H NMR (500 MHz, CDCl₃) δ 8.36 (d, J=2.8 Hz, 1H), 8.03 (dd, J=8.9, 2.8 Hz, 1H), 7.00 (d, J=8.7 Hz, 1H), 3.63 (t, J=6.4 Hz, 2H), 3.44 (s, 2H), 3.07 (d, J=4.9 Hz, 4H), 2.57 (s, 4H), 2.48-2.41 (m, 2H), 2.35 (s, 3H), 2.17 (s, 3H), 1.73-1.64 (m, 2H), 0.83 (d, J=0.7 Hz, 9H), −0.00 (d, J=0.7 Hz, 6H).

### 3-(((3-((tert-butyldimethylsilyl)oxy)propyl)(methyl)amino)methyl)-4-(4-methylpiperazin-1-yl)aniline

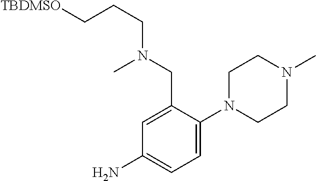 [SMILES: CN1CCN(c2ccc(N)cc2CN(C)CCCO[Si](C)(C)C(C)(C)C)CC1]

General procedure 12 was applied to 3-((tert-butyldimethylsilyl)oxy)-N-methyl-N-(2-(4-methylpiperazin-1-yl)-5-nitrobenzyl)propan-1-amine (580 mg, 1.33 mmol) and palladium on carbon (142 mg, 0.133 mmol) in ethanol (13 mL). The crude material was purified by flash column chromatography eluting with methanol (0-20%) in dichloromethane to yield the title compound as a yellow solid (467 mg, 1.15 mmol, 86%).

(ES, m/z) [M+H]⁺=407.4

¹H NMR (500 MHz, CDCl₃) δ 6.92 (d, J=8.4 Hz, 1H), 6.80 (d, J=2.8 Hz, 1H), 6.52 (dd, J=8.4, 2.8 Hz, 1H), 3.61 (t, J=6.5 Hz, 2H), 3.49 (s, 2H), 2.82 (t, J=4.7 Hz, 4H), 2.62-2.41 (m, 6H), 2.31 (s, 3H), 2.18 (s, 3H), 1.72 (p, J=6.7 Hz, 2H), 0.84 (s, 9H), 0.00 (s, 6H).

### 2-((3-(((3-hydroxypropyl)(methyl)amino)methyl)-4-(4-methylpiperazin-1-yl)phenyl)amino)-5-((triisopropylsilyl)ethynyl)pyrido[2,3-d]pyrimidin-7(8H)-one

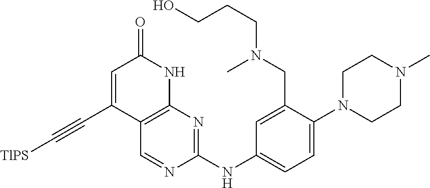

General procedure 13 was applied to 2-(methylsulfonyl)-5-((triisopropylsilyl)ethynyl)pyrido[2,3-d]pyrimidin-7(8H)-one (424 mg, 1.05 mmol), 3-(((3-((tert-butyldimethylsilyl)oxy)propyl)(methyl)amino)methyl)-4-(4-methylpiperazin-1-yl)aniline (467 mg, 1.15 mmol) and trifluoroacetic acid (96.0 μL, 1.25 mmol) in acetonitrile (10 mL). The crude material was purified by flash column chromatography eluting with methanol (0-5%) in dichloromethane to yield the title compound as a yellow solid (81.4 mg, 0.132 mmol, 12%).

(ES, m/z) [M+H]⁺=618.4

### 5-Methyl-3⁴-(4-methylpiperazin-1-yl)-1⁵-((triisopropylsilyl)ethynyl)-1⁷,1⁸-dihydro-2,5-diaza-1(2,8)-pyrido[2,3-d]681yrimidine-3(1,3)-benzenacyclooctaphan-1⁷-one

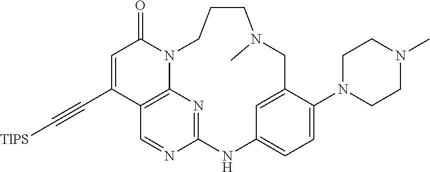

General procedure 20 was applied to 2-((3-(((3-hydroxypropyl)(methyl)amino)methyl)-4-(4-methylpiperazin-1-yl)phenyl)amino)-5-((triisopropylsilyl)ethynyl)pyrido[2,3-d]pyrimidin-7(8H)-one (75.0 mg, 0.122 mmol), triphenylphosphine (127 mg, 0.486 mmol) and DIAD (91.0 μL, 0.486 mmol) in THF (5.0 mL). The crude material was purified by flash column chromatography eluting with methanol (0-25%) in dichloromethane to yield the title compound as a yellow solid (36.6 mg, 61.1 μmol, 50%).

(ES, m/z) [M+H]⁺=600.5

### 1⁵-Ethynyl-5-methyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphan-1⁷-one

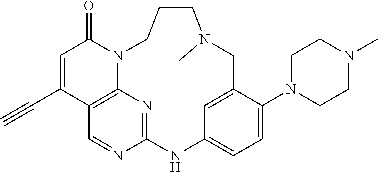 [SMILES: C#Cc1cc(=O)n2c3nc(ncc13)Nc1ccc(N3CCN(C)CC3)c(c1)CN(C)CCC2]

General procedure 15 was applied to 5-methyl-3⁴-(4-methylpiperazin-1-yl)-15-((triisopropylsilyl)ethynyl)-1⁷,1⁸-dihydro-2,5-diaza-1(2,8)-pyrido[2,3-d]pyrimidine-3(1,3)-benzenacyclooctaphan-1⁷-one (70.0 mg, 116 μmol) and potassium fluoride (135 mg, 2.30 mmol) in DMF (1.2 mL). The crude material was purified by flash column chromatography eluting with methanol (0-50%) in dichloromethane, followed by trituration with diethyl ether to yield the title compound as a yellow solid (13.6 mg, 30.7 μmol, 26%).

(ES, m/z) [M+H]⁺=444.3

¹H NMR (500 MHz, CDCl₃) δ 8.74 (d, J=1.2 Hz, 1H), 8.26 (s, 1H), 7.00 (d, J=8.4 Hz, 1H), 6.89 (dd, J=8.4, 2.6 Hz, 1H), 6.49 (s, 1H), 4.11 (s, 2H), 3.64 (s, 2H), 3.27 (p, J=1.6 Hz, 1H), 2.87 (d, J=4.9 Hz, 4H), 2.59 (s, 4H), 2.47 (s, 2H), 2.44 (s, 3H), 2.33 (s, 3H), 2.04-1.88 (m, 2H).

### Example 227

### 2-(2-Fluoro-5-nitrophenyl)acetamide

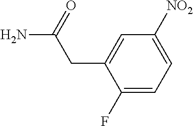 [SMILES: NC(=O)Cc1cc([N+](=O)[O-])ccc1F]

A mixture of (2-fluoro-5-nitrophenyl)acetic acid (24.0 g, 121 mmol, 1.0 eq.), ammonium chloride (9.67 g, 181 mmol, 1.5 eq.), PyBOP (94.1 g, 181 mmol, 1.5 eq.) and N-methylmorpholine (26.5 mL, 241 mmol, 2.0 eq.) in DMF (20 mL) was stirred for 24 hours at room temperature. The resulting mixture was diluted with water (100 mL) and the precipitated solids collected by filtration and washed with water (3×50 mL) to yield the title compound as a white solid (16.0 g, 67%).

### 2-(2-Fluoro-5-nitrophenyl)ethanamine

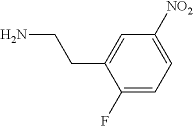 [SMILES: NCCc1cc([N+](=O)[O-])ccc1F]

To a stirred solution of 2-(2-fluoro-5-nitrophenyl)acetamide (16.0 g, 80.7 mmol, 1.0 eq.) in THF (160 mL) was added sodium borohydride (9.16 g, 242 mmol, 3.0 eq.) and boron trifluoride diethyl etherate (17.9 g, 242 mmol, 3.0 eq.) in portions at 0° C. The resulting mixture was stirred for 1 hour at room temperature then concentrated under reduced pressure. The reaction was quenched with 1M aqueous hydrochloric acid (1 M) at 0° C. then basified to pH 9 with 1M aqueous sodium hydroxide. The resulting mixture was extracted with ethyl acetate (2×300 mL). The combined organic layers were washed with saturated aqueous sodium hydrogen carbonate (2×100 mL), dried (Na₂SO₄) and concentrated under reduced pressure to yield the title compound as a white solid (18.0 g, crude).

### Benzyl 2-[(tert-butyldiphenylsilyl)oxy]acetate

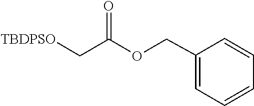

To a stirred solution of benzyl 2-hydroxyacetate (30.0 g, 181 mmol, 1.0 eq.) in DMF (200 mL) at 0° C. was added imidazole (24.6 g, 361 mmol, 2.0 eq.) in DMF (150 mL) dropwise over 2 minutes followed by tert-Butyldiphenylsilyl chloride (59.6 g, 217 mmol, 1.2 eq.) in DMF (150 mL) dropwise over 2 minutes. The resulting mixture was stirred for 24 hours at room temperature then diluted with water (20 mL) and extracted with ethyl acetate (3×200 mL). The combined organic layers were washed with brine (3×50 mL), dried (Na₂SO₄) and concentrated under reduced pressure. The residue was purified by reverse phase flash column chromatography eluting with acetonitrile (10-100%) in water (0.1% formic acid), to yield the title compound as a colourless oil (40.0 g, 55%).

### [(tert-Butyldiphenylsilyl)oxy]acetic acid

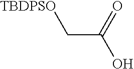

To a stirred solution of benzyl 2-[(tert-butyldiphenylsilyl)oxy]acetate (15.0 g, 37.1 mmol, 1.0 eq.) in methanol (50 mL) was added palladium on carbon (10%, 1.0 g). The mixture was hydrogenated at room temperature under 30 psi of hydrogen pressure overnight, filtered through a Celite pad and concentrated under reduced pressure to yield the title compound as a colourless solid (13.0 g, crude).

### 2-[2-(4-Methylpiperazin-1-yl)-5-nitrophenyl]ethanamine

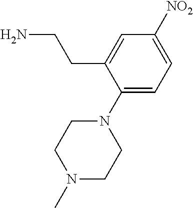 [SMILES: CN1CCN(c2ccc([N+](=O)[O-])cc2CCN)CC1]

A solution of 2-(2-fluoro-5-nitrophenyl)ethanamine (6.00 g, 32.6 mmol, 1.0 eq.) in N-methylpiperazine (60 mL) was stirred for 3 hours at 80° C. The resulting mixture was extracted with ethyl acetate (2×200 mL). The combined organic layers were washed with brine (2×100 mL), dried (Na₂SO₄) and concentrated under reduced pressure to yield the title compound as a yellow solid (6.00 g, 69%).

### 2-[(tert-Butyldiphenylsilyl)oxy]-N-(2-[2-(4-methylpiperazin-1-yl)-5-nitrophenyl]ethylacetamide

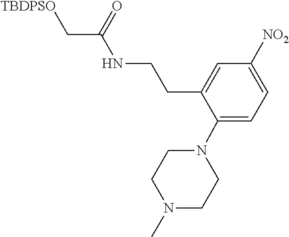

To a stirred solution of 2-[2-(4-methylpiperazin-1-yl)-5-nitrophenyl]ethanamine (6.00 g, 22.7 mmol, 1.0 eq.) and [(tert-butyldiphenylsilyl)oxy]acetic acid (8.57 g, 27.2 mmol, 1.2 eq.) in dichloromethane (60 mL) was added HOBT (3.68 g, 27.2 mmol, 1.2 eq.), EDCl (5.22 g, 27.2 mmol, 1.2 eq.) and triethylamine (6.89 g, 68.1 mmol, 3.0 eq.). The resulting mixture was stirred overnight at room temperature. The residue was purified by flash column chromatography eluting with methanol (13%) in dichloromethane to afford the title compound as a yellow solid (7.00 g, 55%).

### N-(2-[5-Amino-2-(4-methylpiperazin-1-yl)phenyl]ethyl-2-[(tert-butyldiphenylsilyl)oxy]acetamide

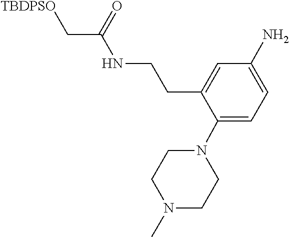

General procedure 12 was applied to 2-[(tert-butyldiphenylsilyl)oxy]-N-(2-[2-(4-methylpiperazin-1-yl)-5-nitrophenyl]ethylacetamide (7.00 g, 12.5 mmol) and palladium on carbon (1.40 g, 13.2 mmol) in THF (70 mL) to yield the title compound as a light brown solid (6.00 g, 90%).

### 2-[(tert-Butyldiphenylsilyl)oxy]-N-(2-[2-(4-methylpiperazin-1-yl)-5-((7-oxo-5-[2-(triisopropylsilyl)ethynyl]-8H-pyrido[2,3-d]pyrimidin-2-ylamino)phenyl]ethylacetamide

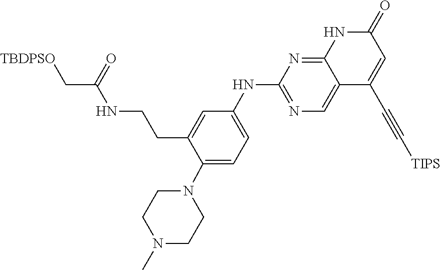

General procedure 13 was applied to N-(2-[5-amino-2-(4-methylpiperazin-1-yl)phenyl]ethyl-2-[(tert-butyldiphenylsilyl)oxy]acetamide (6.00 g, 11.3 mmol), 2-methanesulfonyl-5-[2-(triisopropylsilyl)ethynyl]-8H-pyrido[2,3-d]pyrimidin-7-one (6.88 g, 16.9 mmol) and trifluoroacetic acid (2.58 g, 22.6 mmol) in 2-butyl alcohol (60 mL) stirred for 16 hours at 80° C. The residue was purified by flash column chromatography eluting with methanol (16%) in dichloromethane to afford the title compound as a brown solid (4.00 g, 41%).

### 2-Hydroxy-N-(2-[2-(4-methylpiperazin-1-yl)-5-((7-oxo-5-[2-(triisopropylsilyl)ethynyl]-8H-pyrido[2,3-d]pyrimidin-2-ylamino)phenyl]ethylacetamide

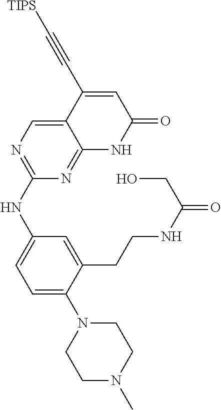

A solution of 2-[(tert-butyldiphenylsilyl)oxy]-N-(2-[2-(4-methylpiperazin-1-yl)-5-((7-oxo-5-[2-(triisopropylsilyl)ethynyl]-8H-pyrido[2,3-d]pyrimidin-2-ylamino)phenyl]ethylacetamide (4.00 g, 4.67 mmol, 1.0 eq.) in hydrogen chloride in methanol (4 M, 40 mL) was stirred for 14 hours at room temperature. The residue was purified by reverse phase flash column chromatography eluting with methanol (30-100%) in water (0.1% trifluoroacetic acid), to yield the title compound as a yellow solid (500 mg, 17%).

### 3⁴-(4-Methylpiperazin-1-yl)-1⁵-((triisopropylsilyl)ethynyl)-1⁷,1⁸-dihydro-2,6-diaza-1(2,8)-pyrido[2,3-d]pyrimidina-3(1,3)-benzenacyclooctaphane-1⁷,7-dione

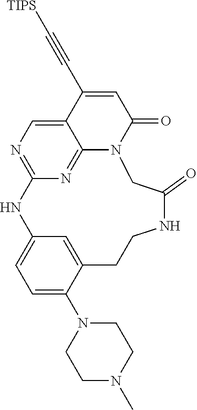

General procedure 20 was applied to 2-hydroxy-N-(2-[2-(4-methylpiperazin-1-yl)-5-((7-oxo-5-[2-(triisopropylsilyl)ethynyl]-8H-pyrido[2,3-d]pyrimidin-2-ylamino)phenyl]ethylacetamide (160 mg, 0.259 mmol), triphenylphosphine (272 mg, 1.04 mmol) and DEAD (180 mg, 1.04 mmol in dichloromethane (2.0 mL). The residue was purified by reverse phase flash column chromatography eluting with methanol (30-100%) in water (0.1% trifluoroacetic acid) to yield the title compound as a yellow solid (60.0 mg, 38%).

### 1⁵-Ethynyl-3⁴-(4-Methylpiperazin-1-yl)-1⁷,1⁸-dihydro-2,6-diaza-1(2,8)-pyrido[2,3-d]pyrimidina-3(1,3)-benzenacyclooctaphane-1⁷,7-dione

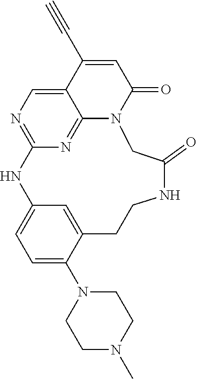 [SMILES: C#Cc1cc(=O)n2c3nc(ncc13)Nc1ccc(N3CCN(C)CC3)c(c1)CCNC(=O)C2]

General procedure 15 was applied to 3⁴-(4-Methylpiperazin-1-yl)-1⁵-((triisopropylsilyl)ethynyl)-1⁷,1⁸-dihydro-2,6-diaza-1(2,8)-pyrido[2,3-d]pyrimidina-3(1,3)-benzenacyclooctaphane-1⁷,7-dione (60.0 mg, 0.100 mmol) and potassium fluoride (58.1 mg, 1.00 mmol) in THF (0.50 mL), DMF (0.50 mL) and water (50 μL) stirred for 1 hour at room temperature. The crude product was purified by Prep-HPLC eluting with acetonitrile (5-15%) in water (0.1% formic acid), to yield the title compound as a yellow solid (13.0 mg, 29%).

(ES, m/z) [M+H]⁺=444.40

¹H-NMR (400 MHz, DMSO-d₆) δ 10.26 (s, 1H), 8.83 (s, 1H), 8.41 (d, J=6.4 Hz, 1H), 8.14 (d, J=8.5 Hz, 2H), 7.10 (d, J=2.8 Hz, 2H), 6.63 (s, 1H), 5.10 (s, 1H), 5.00 (s, 2H), 2.84-2.68 (m, 8H), 2.45 (s, 6H).

### Example 228

### Methyl 3-((tert-butyldiphenylsilyl)oxy)butanoate

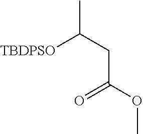

General procedure 21 was applied to methyl 3-hydroxybutanoate (1.75 g, 14.8 mmol), tert-butyldiphenylsilyl chloride (4.70 mL, 17.8 mmol) and imidazole (1.51 g, 22.2 mmol) in DMF (10 mL). The crude product was purified by flash column chromatography eluting with ethyl acetate (25%) in petroleum ether to afford the title compound as a colourless oil (2.23 g, 42%).

¹H NMR (500 MHz, CDC₃) 67.80-7.44 (4H, m), 7.53-7.40 (6H, m), 4.32 (2H, s), 3.73 (3H, s), 1.17 (9H, s).

### 3-((tert-Butyldiphenylsilyl)oxy)butanal

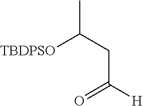

To a stirred solution of methyl 3-((tert-butyldiphenylsilyl)oxy)butanoate (2.23 g, 6.27 mmol) in dichloromethane (15 mL) was added DIBAL-H (6.90 mL, 6.90 mmol) dropwise at −78° C. and left to stir for 15 mins. The reaction was quenched with dropwise addition of saturated aqueous ammonium chloride (10 mL) and extracted with diethyl ether (100 mL), dried (MgSO₄), filtered through celite and concentrated under reduced pressure to afford the title compound as a colourless oil (2.08 g, 98%)

¹H NMR (500 MHz, CDC₃) δ 9.63 (1H, dd, J=3.0 and 2.1 Hz), 7.62-7.54 (4H, m), 7.34-7.21 (6H, m), 4.29-4.19 (1H, m), 2.41 (1H, ddd, J=15.8, 6.1 and 3.0 Hz), 2.34 (1H, ddd, J=15.8, 5.5 and 2.1 Hz), 1.06 (3H, d, J=6.2 Hz), 0.94 (9H, s).

### Ethyl (E)-5-((tert-butyldiphenylsilyl)oxy)hex-2-enoate

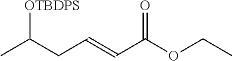

To a stirred solution of triethylphosphonoacetate (1.70 mL, 8.68 mmol), DBU (1.00 mL, 7.01 mmol) and lithium chloride (325 mg, 7.66 mmol) was added 3-((tert-butyldiphenylsilyl)oxy)butanal (2.08 g, 6.38 mmol) and left to stir for 18 hours at room temperature. The reaction mixture was diluted in ethyl acetate (30 mL), then washed with water (10 mL), brine (10 mL), dried (MgSO₄) and concentrated under reduced pressure. The crude product was purified by flash column chromatography eluting with ethyl acetate (10%) in petroleum ether to afford the title compound as a colourless oil (1.49 g, 59%).

¹H NMR (500 MHz, CDCl₃) δ7.62-7.52 (4H, m), 7.32-7.17 (6H, m), 6.83 (1H, dt, J=15.3 and 7.5 Hz), 5.66 (1H, dt, J=15.3 and 1.4 Hz), 4.11-3.96 (2H, m), 3.90-3.80 (2H, m), 1.15-1.11 (3H, m), 0.97 (3H, d, J=6.7 Hz), 0.95 (9H, s).

### Ethyl 5-((tert-butyldiphenylsilyl)oxy)hexanoate

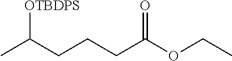

General procedure 12 was applied to ethyl (E)-5-((tert-butyldiphenylsilyl)oxy)hex-2-enoate (1.49 g, 3.76 mmol) and palladium on carbon (149 mg, 1.41 mmol) in ethanol (10 mL), stirred for 3 hours at room temperature under an hydrogen atmosphere. The resulting mixture was filtered, washing with ethanol (3×2.0 mL). The filtrate was concentrated under reduced pressure to afford the title compound as a colourless oil (1.41 g, 94%).

¹H NMR (500 MHz, CDCl₃) δ7.61-7.54 (4H, m), 7.31-7.19 (6H, m), 3.97 (2H, q, J=7.1 Hz), 3.80-3.70 (1H, m), 2.07 (2H, t, J=7.5 Hz), 1.61-1.46 (1H, m), 1.45-1.26 (2H, m), 1.09 (3H, t, J=7.1 Hz), 0.96-0.94 (12H, m).

### 5-((tert-Butyldiphenylsilyl)oxy)hexan-1-ol

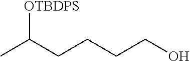

To a stirred solution of ethyl 5-((tert-butyldiphenylsilyl)oxy)hexanoate (1.41 g, 3.55 mmol) in THF (10 mL) was added lithium aluminium hydride (1M, 4.2 ml, 4.20 mmol) dropwise at 0° C. and left to stir for 2 hours. The reaction mixture was extracted with ethyl acetate (20 mL), then washed with water (10 mL), brine (10 mL), dried (MgSO₄) and concentrated under reduced pressure to give the title compound as a colourless oil (1.14 g, 90%). No further purification was required.

¹H NMR (500 MHz, CDCl₃) δ7.63-7.53 (4H, m), 7.32-7.20 (6H, m), 3.79-3.70 (1H, m), 3.39 (2H, t, J=6.6 Hz), 1.46-1.14 (6H, m), 1.00-0.93 (12H, m).

### tert-Butyl((6-(2-fluoro-5-nitrophenoxy)hexan-2-yl)oxy)diphenylsilane

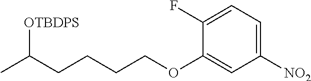

General procedure 22 was applied to 2-fluoro-5-nitrophenol (457 mg, 2.90 mmol), 5-((tert-Butyldiphenylsilyl)oxy)hexan-1-ol (1.14 g, 3.20 mmol), DIAD (745 μL, 3.80 mmol) and triphenylphosphine (988 mg, 3.80 mmol) in THF (7.2 mL). The crude product was purified by flash column chromatography eluting with ethyl acetate (10%) in petroleum ether to afford the title compound as a colourless oil (1.15 g, 80%).

¹H NMR (500 MHz, CDC₃) δ 7.72-7.65 (2H, m), 7.62-7.53 (4H, m), 7.31-7.18 (6H, m), 7.06-6.98 (1H, m), 3.85 (2H, t, J=6.5 Hz), 3.82-3.73 (1H, m), 1.66-1.55 (2H, m), 1.46-1.32 (4H, m), 0.99 (3H, d, J=6.1 Hz), 0.94 (9H, s).

### 1-(2-((5-((tert-Butyldiphenylsilyl)oxy)hexyl)oxy)-4-nitrophenyl)-4-methylpiperazine

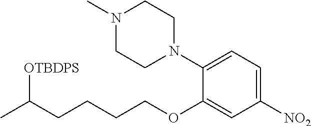

General procedure 11 was applied to tert-butyl((6-(2-fluoro-5-nitrophenoxy)hexan-2-yl)oxy)diphenylsilane (1.09 g, 2.20 mmol), N-methylpiperizine (489 μL, 4.40 mmol), potassium carbonate (457 mg, 3.30 mmol) in DMF (20 mL). The crude product was purified by flash column chromatography eluting with methanol (10%) in dichloromethane to afford the title compound as a yellow oil (1.15 g, 90%).

¹H NMR (500 MHz, DMSO-d₆) δ 7.82 (1H, dd, J 8.9 and 2.5 Hz), 7.64-7.58 (5H, m), 7.46-7.36 (6H, m), 6.98 (1H, d, J=8.9 Hz), 4.08-3.95 (3H, m), 3.18-3.15 (4H, m), 2.43-2.34 (4H, m), 2.16 (3H, s), 1.70-1.60 (2H, m), 1.54-1.40 (4H, m), 1.04 (3H, d, J=6.1 Hz), 0.99 (9H, s).

### 3-((5-((tert-Butyldiphenylsilyl)oxy)hexyl)oxy)-4-(4-methylpiperazin-1-yl)aniline

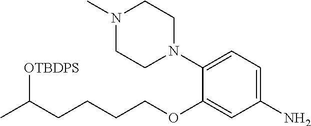

General procedure 12 was applied to 1-(2-((5-((tert-butyldiphenylsilyl)oxy)hexyl)oxy)-4-nitrophenyl)-4-methylpiperazine (1.15 g, 2.00 mmol) and palladium on carbon (115 mg, 1.08 mmol) in ethanol (10 mL), stirred for 3 hours at room temperature under an hydrogen atmosphere. The resulting mixture was filtered, washing with ethanol (3×2 mL). The filtrate was concentrated under reduced pressure to afford the title compound as a brown oil (1.08 g, 99%).

¹H NMR (500 MHz, DMSO-d₆) δ 7.62-7.56 (4H, m), 7.46-7.35 (6H, m), 6.54 (1H, d, J=8.3 Hz), 6.14 (1H, d, J=2.4 Hz), 6.03 (1H, dd, J=8.3 and 2.4 Hz), 4.65 (2H, s), 3.87-3.79 (1H, m), 3.72 (2H, t, J=6.2 Hz), 2.81-2.69 (4H, m), 2.38-2.26 (4H, m), 2.12 (3H, s), 1.63-1.35 (6H, s), 1.01 (3H, d, J=6.1 Hz), 0.98 (9H, s).

### 9-Methyl-3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclononaphan-17-one

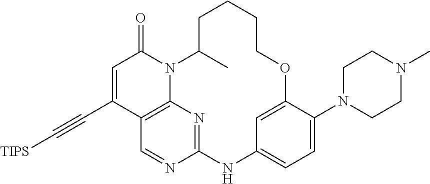

General procedure 13 was applied to 3-((5-((tert-butyldiphenylsilyl)oxy)hexyl)oxy)-4-(4-methylpiperazin-1-yl)aniline (721 mg, 1.32 mmol) with 2-(methylsulfonyl)-5-((triisopropylsilyl)ethynyl)pyrido[2,3-d]pyrimidin-7(8H)-one (486 mg, 1.20 mmol) and trifluoroacetic acid (110 μL, 1.44 mmol) in acetonitrile (10 mL). The crude material was purified by flash column chromatography eluting with methanol (10%) in dichloromethane to yield the crude 2-((3-((5-hydroxyhexyl)oxy)-4-(4-methylpiperazin-1-yl)phenyl)amino)-5-((triisopropylsilyl)ethynyl)pyrido[2,3-d]pyrimidin-7(8H)-one.

According to general procedure 20 2-((3-((5-hydroxyhexyl)oxy)-4-(4-methylpiperazin-1-yl)phenyl)amino)-5-((triisopropylsilyl)ethynyl)pyrido[2,3-d]pyrimidin-7(8H)-one (221 mg, 0.35 mmol), DIAD (274 μL, 1.40 mmol) and triphenylphosphine (367 mg, 1.40 mmol) in THF (14.6 mL). The crude product was purified by flash column chromatography eluting with methanol (10%) in dichloromethane to afford the title compound as a yellow solid (40.0 mg, 19%).

¹H NMR (500 MHz, CDC₃) δ 8.85 (1H, s), 8.13 (1H, d, J=2.3 Hz), 7.60 (1H, s), 6.89 (1H, d, J=8.3 Hz), 6.55-6.49 (2H, m), 5.94-5.78 (1H, m), 4.32-4.18 (2H, m), 3.30-3.13 (2H, m), 3.02-2.87 (2H, m), 2.70-2.57 (4H, m), 2.36 (3H, s), 1.89-1.63 (4H, m), 1.58-1.46 (5H, m), 1.21-1.09 (21H, m).

### 1⁵-Ethynyl-9-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclononaphan-17-one

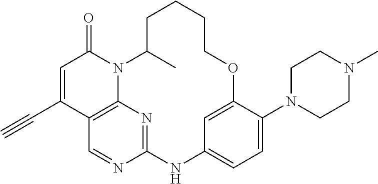 [SMILES: C#Cc1cc(=O)n2c3nc(ncc13)Nc1ccc(N3CCN(C)CC3)c(c1)OCCCCC2C]

General procedure 15 was applied to 9-methyl-3⁴-(4-methylpiperazin-1-yl)-15-((triisopropylsilyl)ethynyl)-1⁷,1⁸-dihydro-4-oxa-2-aza-1(2,8)-pyrido[2,3-d]pyrimidina-3(1,3)-benzenacyclononaphan-17-one (40.0 mg, 70.0 μmol) with potassium fluoride (40.0 mg, 0.651 mmol) in DMF (2.0 mL). The crude material was purified by flash column chromatography eluting with methanol (10%) in dichloromethane to yield the title compound as a yellow solid (40.0 mg, 44%).

Major and minor enantiomer reported in a ratio of 80:20

(ES, m/z): [M+1]+=459

¹H-NMR (500 MHz, CDCl₃) δ 1.39 (1.2H, d, J=6.5 Hz), 1.51 (1.8H, d, J=6.8 Hz), 1.53-1.58 (1H, m), 1.68-1.72 (1H, m), 1.91-1.83 (2H, m), 2.08-2.19 (1H, m), 2.39 (3H, s), 2.67 (4H, br. s), 2.93 (2H, br. s), 3.24 (2H, br. s), 4.14-4.34 (2H, m), 5.30 (1H, s), 5.42-5.51 (0.2H, m), 5.84-5.92 (0.8H, m), 6.49-5.57 (1.8H, m), 6.76 (0.2H, s), 6.83 (0.2H, d, J=8.3 Hz), 6.91 (0.8H, d, J=8.4 Hz), 7.39 (0.2H, s), 7.62 (0.8H, s), 8.14 (0.8H, d, J=2.4 Hz), 8.42 (0.2H, d, J=2.4 Hz), 8.83 (0.8H, s), 9.14 (0.2H, s)

### Example 229

### 5-[(tert-Butyldiphenylsilyl)oxy]pentan-1-ol

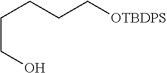

A solution of 1,5-pentanediol (3.00 g, 28.8 mmol, 1.0 eq.), imidazole (2.94 g, 43.2 mmol, 1.5 eq.) and tert-butyldiphenylsilyl chloride (7.92 g, 28.8 mmol, 1.0 eq.) in DMF (30 mL) was stirred overnight at room temperature. The resulting mixture was diluted with water (150 mL) and extracted with ethyl acetate (3×200 mL). The combined organic layers were concentrated under reduced pressure. The residue was purified by flash column chromatography eluting with ethyl acetate (9%) in 40-60 petroleum ether to afford the title compound as a colourless oil (3.30 g, 33%).

### 1-[2-({5-[(tert-Butyidiphenylsilyl)oxy]pentyl}oxy)-4-nitrophenyl]-4-methylpiperazine

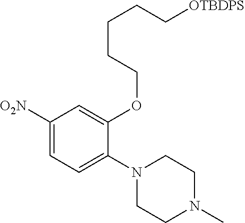

A solution of 5-[(tert-butyldiphenylsilyl)oxy]pentan-1-ol (3.00 g, 8.76 mmol, 1.0 eq.), 2-(4-methylpiperazin-1-yl)-5-nitrophenol (2.29 g, 9.63 mmol, 1.1 eq.), triphenylphosphine (4.59 g, 17.5 mmol, 2.0 eq.) and DEAD (3.05 g, 17.5 mmol, 2.0 eq.) in THF (30 mL) was stirred for 1 hour at room temperature. The reaction was quenched by the addition of methanol (10 mL) at 0° C. The resulting mixture was concentrated under reduced pressure.

The residue was purified by flash column chromatography eluting with ethyl acetate (9%) in 40-60 petroleum ether to afford the title compound as a yellow solid (3.00 g, 61%).

### 3-({5-[(tert-Butyldiphenylsilyl)oxy]pentyl}oxy)-4-(4-methylpiperazin-1-yl)aniline

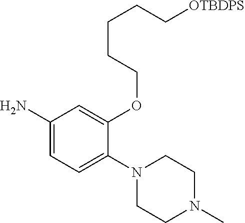

General procedure 12 was applied to 1-[2-({5-[(tert-butyldiphenylsilyl)oxy]pentyl}oxy)-4-nitrophenyl]-4-methylpiperazine (3.00 g, 5.34 mmol) and palladium on carbon (10%, 570 mg) in methanol (30 mL). The mixture was hydrogenated at room temperature under 30 psi of hydrogen pressure overnight, to yield the title compound as a light yellow oil (2.42 g, 85%).

### 2-{[3-({5-[(tert-Butyldiphenylsilyl)oxy]pentyl}oxy)-4-(4-methylpiperazin-1-yl)phenyl]amino}-6-methyl-5-[2-(triisopropyisiyl)ethynyl]-8H-pyrido[2,3-d]pyrimidin-7-one

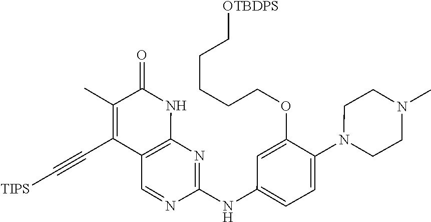

General procedure 13 was applied to 2-methanesulfinyl-6-methyl-5-[2-(triisopropylsilyl)ethynyl]-8H-pyrido[2,3-d]pyrimidin-7-one (1.00 g, 2.48 mmol), 3-({5-[(tert-butyldiphenylsilyl)oxy]pentyl}oxy)-4-(4-methylpiperazin-1-yl)aniline (1.34 g, 2.53 mmol) and trifluoroacetic acid (0.550 mL, 7.43 mmol) in dioxane (10 mL), stirred for 24 hours at 100° C. The residue was purified by reverse phase flash column chromatography eluting with acetonitrile (10-60%) in water (0.1% formic acid), to yield the title compound as a yellow solid (1.00 g, 46%).

### 2-({3-[(5-Hydroxypentyl)oxy]-4-(4-methylpiperazin-1-yl)phenyl}amino)-6-methyl-5-[2-(triisopropylsilyl)ethynyl]-8H-pyrido[2,3-d]pyrimidin-7-one

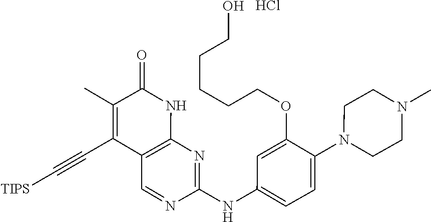

A solution of 2-{[3-({5-[(tert-butyldiphenylsilyl)oxy]pentyl}oxy)-4-(4-methylpiperazin-1-yl)phenyl]amino}-6-methyl-5-[2-(triisopropylsilyl)ethynyl]-8H-pyrido[2,3-d]pyrimidin-7-one (1.00 g, 1.15 mmol, 1.0 eq.) in hydrogen chloride in methanol (20 mL) was stirred for 14 hours at 60° C. The mixture was allowed to cool down to room temperature then concentrated under reduced pressure to yield the title compound as a yellow solid (650 mg, 89%).

### 1⁶-Methyl-3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclononaphan-1⁷-one

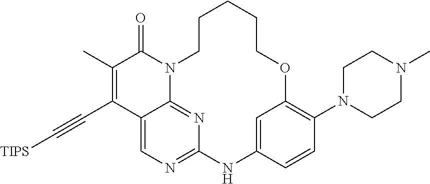

General procedure 20 was applied to 2-({3-[(5-hydroxypentyl)oxy]-4-(4-methylpiperazin-1-yl)phenyl}amino)-6-methyl-5-[2-(triisopropylsilyl)ethynyl]-8H-pyrido[2,3-d]pyrimidin-7-one (300 mg, 0.474 mmol), triphenylphosphine (373 mg, 1.42 mmol) and DEAD (247 mg, 1.42 mmol) in THF (5.0 mL). The residue was purified by reverse phase flash column chromatography eluting with acetonitrile (10-50%) in water (0.1% formic acid), to yield the title compound as a yellow solid (148 mg, 51%).

### 1⁵-Ethynyl-1⁶-Methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclononaphan-1⁷-one

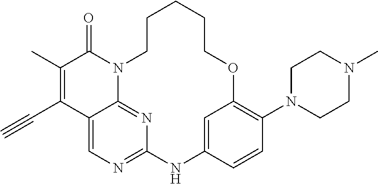 [SMILES: C#Cc1c(C)c(=O)n2c3nc(ncc13)Nc1ccc(N3CCN(C)CC3)c(c1)OCCCCC2]

General procedure 15 was applied to 1⁶-methyl-3⁴-(4-methylpiperazin-1-yl)-15-((triisopropylsilyl)ethynyl)-1⁷,1⁸-dihydro-4-oxa-2-aza-1(2,8)-pyrido[2,3-d]pyrimidina-3(1,3)-benzenacyclononaphan-1⁷-one (100 mg, 0.163 mmol) and potassium fluoride (47.2 mg, 0.815 mmol) in DMF (1.0 mL), stirred for 1 hour at room temperature. The residue was purified by reverse phase flash column chromatography eluting with acetonitrile (10-50%) in water (0.1% formic acid), to yield the title compound as a yellow solid (19.7 mg, 26%).

(ES, m/z) [M+H]⁺=459.15

¹H-NMR (300 MHz, DMSO-d₆) δ 10.16 (d, J=15.8 Hz, 1H), 8.79 (d, J=4.7 Hz, 1H), 8.07 (s, 1H), 6.87 (d, J=8.7 Hz, 1H), 6.73 (d, J=9.4 Hz, 1H), 5.23 (d, J=3.5 Hz, 1H), 4.21 (s, 4H), 3.55 (s, 1H), 3.20 (s, 1H), 3.02 (s, 3H), 2.81 (s, 4H), 2.24 (s, 3H), 1.85 (d, J=29.8 Hz, 4H), 1.52 (s, 2H).

### Example 230

### 6-[(tert-Butyldiphenylsilyl)oxy]hexan-1-ol

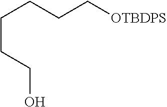

A solution of hexane-1,6-diol (5.00 g, 42.3 mmol, 1.0 eq.), diisopropylethylamine (16.4 g, 127 mmol, 3.0 eq.) and tert-butyldiphenylsilyl chloride (12.8 g, 46.5 mmol, 1.1 eq.) in dichloromethane (50 mL) was stirred overnight at room temperature. The resulting mixture was concentrated under reduced pressure. The residue was purified by reverse-phase flash column chromatography eluting with acetonitrile (30-100%) in water (0.1M ammonium carbonate) to yield the title compound as a colourless oil (5.20 g, 34%).

### 1-[2-((6-[(tert-Butyidiphenylsilyl)oxy]hexyloxy)-4-nitrophenyl]-4-methylpiperazine

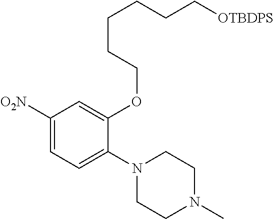

General procedure 22 was applied to 6-[(tert-butyldiphenylsilyl)oxy]hexan-1-ol (5.00 g, 14.0 mmol), 2-(4-methylpiperazin-1-yl)-5-nitrophenol (3.66 g, 15.4 mmol), triphenylphosphine (7.36 g, 28.0 mmol) and DIAD (5.67 g, 28.0 mmol) in dichloromethane (50 mL). The residue was purified by flash column chromatography eluting with ethyl acetate (50%) in 40-60 petroleum ether to afford the title compound as a white solid (5.30 g, 66%).

### 3-((6-((tert-Butyldiphenylsilyl)oxy)hexyl)oxy)-4-(4-methylpiperazin-1-yl)aniline

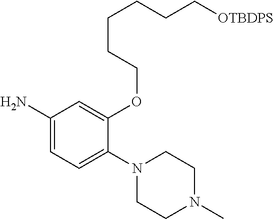

General procedure 12 was applied to 1-[2-((6-[(tert-butyldiphenylsilyl)oxy]hexyloxy)-4-nitrophenyl]-4-methylpiperazine (5.20 g, 9.03 mmol) and palladium on carbon (1.00 g, 9.40 mmol) in THF (52 mL), stirred overnight at room temperature under an hydrogen atmosphere. The crude product was used in the next step directly without further purification.

### 2-([3-((6-[(tert-butyldiphenylsilyl)oxy]hexyloxy)-4-(4-methylpiperazin-1-yl)phenyl]amino-5-[2-(triisopropyisiyl)ethynyl]-8H-pyrido[2,3-d]pyrimidin-7-one

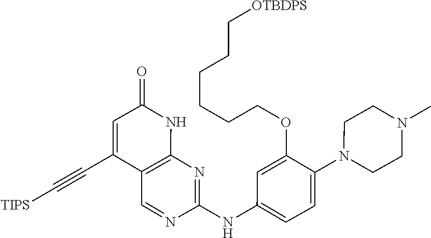

General procedure 13 was applied to 2-methanesulfonyl-5-[2-(triisopropylsilyl)ethynyl]-8H-pyrido[2,3-d]pyrimidin-7-one (5.00 g, 12.3 mmol), trifluoroacetic acid (2.81 g, 24.6 mmol) and 3-((6-[(tert-butyldiphenylsilyl)oxy]hexyloxy)-4-(4-methylpiperazin-1-yl)aniline (7.40 g, 13.6 mmol) in butan-2-ol (50 mL), stirred overnight at 100° C. The residue was purified by flash column chromatography eluting with methanol (18%) in dichloromethane to afford the title compound as a brown solid (4.00 g, 37%).

### 2-((3-[(6-Hydroxyhexyl)oxy]-4-(4-methylpiperazin-1-yl)phenylamino)-5-[2-(triisopropylsilyl)ethynyl]-8H-pyrido[2,3-d]pyrimidin-7-one

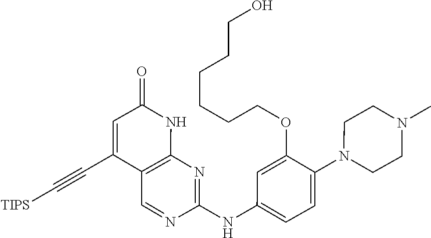

A solution of 2-([3-((6-[(tert-butyldiphenylsilyl)oxy]hexyloxy)-4-(4-methylpiperazin-1-yl)phenyl]amino-5-[2-(triisopropylsilyl)ethynyl]-8H-pyrido[2,3-d]pyrimidin-7-one (3.90 g, 4.47 mmol, 1.0 eq.) in hydrogen chloride in methanol (39 mL) was stirred overnight at room temperature. The residue was purified by reverse-phase flash column chromatography eluting with acetonitrile (30-70%) in water (0.1% trifluoroacetic acid), to afford the title compound as a yellow solid (1.20 g, 42%).

### 3⁴-(4-Methylpiperazin-1-yl)-1⁵-((triisopropylsilyl)ethynyl)-1⁷,1⁸-dihydro-4-oxa-2-aza-1(2, 8)-pyrido[2,3-d]pyrimidina-3(1,3)-benzenacyclodecaphan-1⁷-one

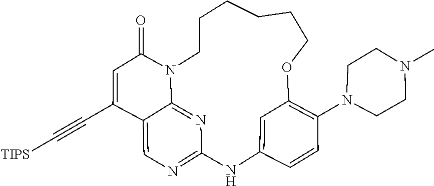

General procedure 20 was applied to 2-((3-[(6-hydroxyhexyl)oxy]-4-(4-methylpiperazin-1-yl)phenylamino)-5-[2-(triisopropylsilyl)ethynyl]-8H-pyrido[2,3-d]pyrimidin-7-one (300 mg, 0.474 mmol), triphenylphosphine (497 mg, 1.89 mmol) and DEAD (330 mg, 1.89 mmol) in THF (3.0 mL). The residue was purified by reverse-phase flash column chromatography eluting with acetonitrile (30-90%) in water (0.1% trifluoroacetic acid) to yield the title compound as a yellow solid (120 mg, 41%).

### 1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclodecaphan-1⁷-one

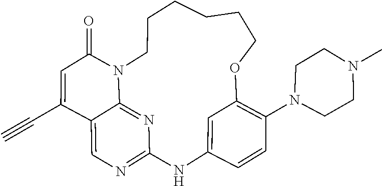 [SMILES: C#Cc1cc(=O)n2c3nc(ncc13)Nc1ccc(N3CCN(C)CC3)c(c1)OCCCCCC2]

General procedure 15 was applied to 3⁴-(4-Methylpiperazin-1-yl)-15-((triisopropylsilyl)ethynyl)-1⁷,1⁸-dihydro-4-oxa-2-aza-1(2,8)-pyrido[2,3-d]pyrimidina-3(1,3)-benzenacyclodecaphan-1⁷-one (100 mg, 0.163 mmol) and potassium fluoride (94.48 mg, 1.63 mmol) in water (50 μL), THF (0.50 mL) and DMF (0.50 mL), stirred for 1 hour at room temperature. The crude product was purified by Prep-HPLC eluting with acetonitrile (22-38%) in water (0.1% formic acid) to afford the title compound as a yellow solid (24.0 mg, 32%).

(ES, m/z): [M+H]⁺=459.20

¹H-NMR (400 MHz, Chloroform-d) δ 8.84 (s, 1H), 8.16 (d, J=2.5 Hz, 1H), 7.47 (s, 1H), 6.92 (d, J=8.4 Hz, 1H), 6.65 (s, 1H), 6.60 (dd, J=8.6, 2.4 Hz, 1H), 4.36 (dd, J=10.5, 6.3 Hz, 2H), 4.24 (t, J=6.5 Hz, 2H), 3.62 (s, 1H), 3.29 (s, 4H), 2.94 (s, 4H), 2.59 (s, 3H), 1.89-1.75 (m, 4H), 1.75-1.64 (m, 4H).

### Example 231

### [2-(4-Methylpiperazin-1-yl)-5-nitrophenyl]methanol

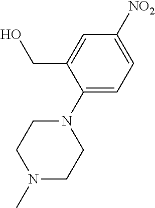 [SMILES: CN1CCN(c2ccc([N+](=O)[O-])cc2CO)CC1]

A solution of (2-fluoro-5-nitrophenyl)methanol (5.00 g, 29.2 mmol, 1.0 eq.) in N-methylpiperazine (20 mL) was stirred for 1 hour at 130° C. The mixture was allowed to cool down to room temperature then diluted with water (100 mL). The precipitated solids were collected by filtration and washed with water (2×10 mL) to yield the title compound as a yellow solid (3.10 g, 42%).

### 1-[2-({4-[(tert-Butyidimethylsilyl)oxy]butoxy}methyl)-4-nitrophenyl]-4-methylpiperazine

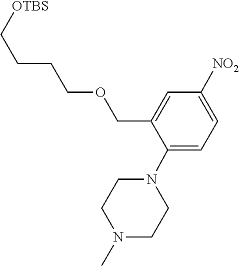 [SMILES: CN1CCN(c2ccc([N+](=O)[O-])cc2COCCCCO[Si](C)(C)C(C)(C)C)CC1]

A solution of [2-(4-methylpiperazin-1-yl)-5-nitrophenyl]methanol (3.10 g, 12.3 mmol, 1.0 eq.) and sodium hydride (380 mg, 16.0 mmol, 1.3 eq.) in DMF (20 mL) was stirred for 1 hour at 0° C. then (4-bromobutoxy)(tert-butyl)dimethylsilane (3.63 g, 13.6 mmol, 1.1 eq.) was added dropwise over 2 minutes at room temperature. The resulting mixture was stirred for 1 hour at room temperature then quenched with saturated aqueous ammonium chloride (50 mL) at 0° C. The aqueous layer was extracted with ethyl acetate (2×50 mL). The combined organic layers were concentrated under reduced pressure. The residue was purified by flash column chromatography eluting with ethyl acetate (12%) in 40-60 petroleum ether to afford the title compound as a yellow solid (2.80 g, 52%).

### 3-({4-[(tert-Butyldimethylsilyl)oxy]butoxy}methyl)-4-(4-methylpiperazin-1-yl)aniline

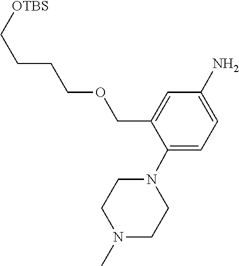 [SMILES: CN1CCN(c2ccc(N)cc2COCCCCO[Si](C)(C)C(C)(C)C)CC1]

General procedure 12 was applied to 1-[2-({4-[(tert-butyldimethylsilyl)oxy]butoxy}methyl)-4-nitrophenyl]-4-methylpiperazine (2.00 g, 4.57 mmol) and palladium on carbon (10%, 390 mg) in methanol (20 mL) to yield the title compound as a light yellow oil (1.45 g, 78%).

### 2-({3-[(4-Hydroxybutoxy)methyl]-4-(4-methylpiperazin-1-yl)phenyl}amino)-5-[2-(triisopropyisiyl)ethynyl]-8H-pyrido[2,3-d]pyrimidin-7-one

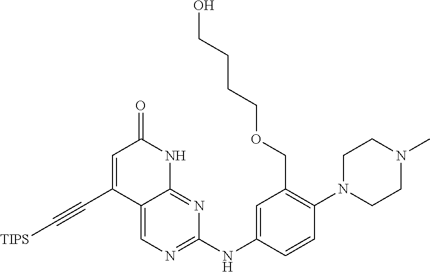

General procedure 13 was applied to 3-({4-[(tert-butyldimethylsilyl)oxy]butoxy}methyl)-4-(4-methylpiperazin-1-yl)aniline (1.30 g, 3.19 mmol), 2-methanesulfonyl-5-[2-(triisopropylsilyl)ethynyl]-8H-pyrido[2,3-d]pyrimidin-7-one (1.55 g, 3.83 mmol) and trifluoroacetic acid (0.470 mL, 6.38 mmol) in 2-methyl-2-butanol (2.0 mL) stirred overnight at 100° C. The residue was purified by reverse phase flash column chromatography eluting with acetonitrile (10-50%) in water (0.1% formic acid), to yield the title compound as a light yellow solid (980 mg, 49%).

### 3⁴-(4-Methylpiperazin-1-yl)-1⁵-((triisopropylsilyl)ethynyl)-1⁷,1⁸-dihydro-5-oxa-2-aza-1(2,8)-pyrido[2,3-d]pyrimidina-3(1,3)-benzenacyclononaphan-1⁷-one

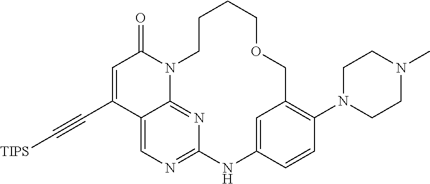

General procedure 20 was applied to 2-({3-[(4-hydroxybutoxy)methyl]-4-(4-methylpiperazin-1-yl)phenyl}amino)-5-[2-(triisopropylsilyl)ethynyl]-8H-pyrido[2,3-d]pyrimidin-7-one (800 mg, 1.29 mmol, 1.0 eq.) and triphenylphosphine (1.69 g, 6.46 mmol) and DEAD (1.13 g, 6.46 mmol) in THF (10 mL). The residue was purified by reverse phase flash column chromatography eluting methanol (10-100%) in water (0.1% formic acid) to yield the title compound as a yellow solid (220 mg, 28%).

### 1⁵-Ethynyl-3⁴-(4-methylpiperazin-1-yl)-1⁷,1⁸-dihydro-5-oxa-2-aza-1(2,8)-pyrido[2,3-d]pyrimidina-3(1,3)-benzenacyclononaphan-1⁷-one

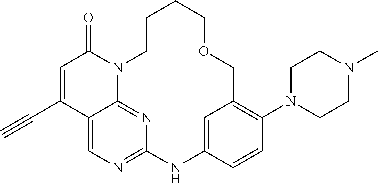 [SMILES: C#Cc1cc(=O)n2c3nc(ncc13)Nc1ccc(N3CCN(C)CC3)c(c1)COCCCC2]

General procedure 15 was applied to 3⁴-(4-methylpiperazin-1-yl)-1⁵-((triisopropylsilyl)ethynyl)-1⁷,1⁸-dihydro-5-oxa-2-aza-1(2,8)-pyrido[2,3-d]pyrimidina-3(1,3)-benzenacyclononaphan-1⁷-one (120 mg, 0.200 mmol) and potassium fluoride (58.0 mg, 1.00 mmol) in DMF (1.2 mL), stirred for 1 hour at 60° C. The precipitated solids were collected by filtration and washed with water (2×10 mL). The residue was purified by reverse phase flash column chromatography eluting with acetonitrile (10-50%) in water (0.1% formic acid), to yield the title compound as a yellow solid (15.7 mg, 17%).

(ES, m/z) [M+H]⁺=445.25

¹H-NMR (300 MHz, DMSO-d₆) δ 10.02 (s, 1H), 8.79 (s, 1H), 8.58 (s, 1H), 7.09 (d, J=2.0 Hz, 2H), 6.52 (s, 1H), 4.87 (s, 1H), 4.65 (s, 2H), 4.32-4.21 (m, 2H), 3.64-3.49 (m, 4H), 2.87-2.75 (m, 4H), 2.26 (s, 3H), 1.81 (d, J=27.1 Hz, 4H).

### Example 232

### 2-(4-Methylpiperazin-1-yl)-5-nitrophenol

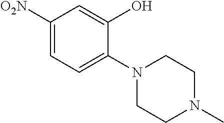 [SMILES: CN1CCN(c2ccc([N+](=O)[O-])cc2O)CC1]

A solution of 2-fluoro-5-nitrophenol (10.0 g, 63.6 mmol, 1.0 eq.) and N,N-diisopropylethylamine (16.6 mL, 95.5 mmol, 1.5 eq.) in N-methylpiperazine (20 mL) was stirred overnight at 80° C. The resulting mixture was extracted with ethyl acetate (3×100 mL).

The combined organic layers were washed with brine (2×200 mL), dried (Na₂SO₄) and concentrated under reduced pressure to yield the title compound as a yellow solid (10.0 g, 66%).

### 1-[2-(2-(2-[(tert-Butyldimethylsilyl)oxy]ethoxyethoxy)-4-nitrophenyl]-4-methylpiperazine

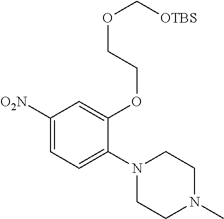 [SMILES: CN1CCN(c2ccc([N+](=O)[O-])cc2OCCOCO[Si](C)(C)C(C)(C)C)CC1]

To a stirred solution of 2-(4-methylpiperazin-1-yl)-5-nitrophenol (2.00 g, 8.43 mmol, 1.0 eq.) and 2-(2-[(tert-butyldimethylsilyl)oxy]ethoxyethanol (2.04 g, 9.27 mmol, 1.1 eq.) in dichloromethane (20 mL) were added triphenylphosphine (4.42 g, 16.9 mmol, 2.0 eq.) and DIAD (3.41 g, 16.9 mmol, 2.0 eq.) 0° C. The resulting mixture was stirred for 1 hour at room temperature. The residue was purified by flash column chromatography eluting with ethyl acetate (50%) in 40-60 petroleum ether to afford the title compound as a yellow solid (2.00 g, 54%).

### 3-(2-(2-[(tert-Butyldimethylsilyl)oxy]ethoxyethoxy)-4-(4-methylpiperazin-1-yl)aniline

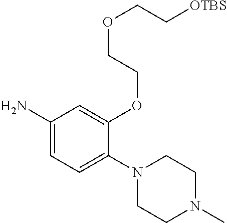 [SMILES: CN1CCN(c2ccc(N)cc2OCCOCCO[Si](C)(C)C(C)(C)C)CC1]

General procedure 12 was applied to 1-[2-(2-(2-[(tert-butyldimethylsilyl)oxy]ethoxyethoxy)-4-nitrophenyl]-4-methylpiperazine (2.00 g, 4.55 mmol) and palladium on carbon (400 mg, 3.76 mmol) in methanol (40 mL) to yield the title compound as a brown solid (1.80 g, 96%).

### 2-((3-[2-(2-Hydroxyethoxy)ethoxy]-4-(4-methylpiperazin-1-yl)phenylamino)-5-[2-(triisopropylsilyl)ethynyl]-8H-pyrido[2,3-d]pyrimidin-7-one

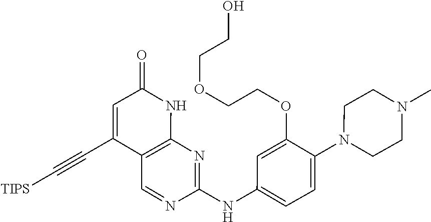

General procedure 13 was applied to 3-(2-(2-[(tert-butyldimethylsilyl)oxy]ethoxyethoxy)-4-(4-methylpiperazin-1-yl)aniline (606 mg, 1.48 mmol), 2-methanesulfonyl-5-[2-(triisopropylsilyl)ethynyl]-8H-pyrido[2,3-d]pyrimidin-7-one (500 mg, 1.23 mmol) and trifluoroacetic acid (281 mg, 2.47 mmol) in butan-2-ol (5.0 mL), stirred overnight at 80° C. The residue was purified by reverse phase flash column chromatography eluting with acetonitrile (30-100%) in water (0.1% trifluoroacetic acid), to yield the title compound as a yellow solid (250 mg, 32%).

### 3⁴-(4-Methylpiperazin-1-yl)-1⁵-((triisopropylsilyl)ethynyl)-1⁷,1⁸-dihydro-4,7-dioxa-2-aza-1(2,8)-pyrido[2,3-d]pyrimidina-3(1,3)-benzenacyclononaphan-1⁷-one

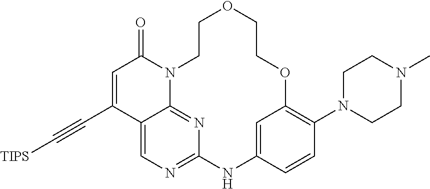

General procedure 20 was applied to 2-((3-[2-(2-hydroxyethoxy)ethoxy]-4-(4-methylpiperazin-1-yl)phenylamino)-5-[2-(triisopropylsilyl)ethynyl]-8H-pyrido[2,3-d]pyrimidin-7-one (250 mg, 0.403 mmol), triphenylphosphine (422 mg, 1.61 mmol) and DEAD (280 mg, 1.61 mmol) in THF (3.0 mL). The residue was purified by reverse phase flash column chromatography eluting with acetonitrile (30-100%) in water (0.1% trifluoroacetic acid), to yield the title compound as a yellow solid (70.0 mg, 29%).

### 1⁵-Ethynyl-3⁴-(4-Methylpiperazin-1-yl)-1⁷,1⁸-dihydro-4,7-dioxa-2-aza-1(2,8)-pyrido[2,3-d]pyrimidina-3(1,3)-benzenacyclononaphan-1⁷-one

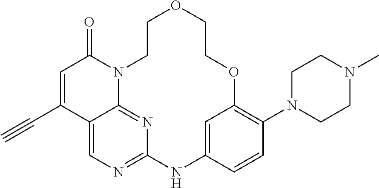 [SMILES: C#Cc1cc(=O)n2c3nc(ncc13)Nc1ccc(N3CCN(C)CC3)c(c1)OCCOCC2]

General procedure 15 was applied to 3⁴-(4-methylpiperazin-1-yl)-15-((triisopropylsilyl)ethynyl)-1⁷,1⁸-dihydro-4,7-dioxa-2-aza-1(2,8)-pyrido[2,3-d]pyrimidina-3(1,3)-benzenacyclononaphan-1⁷-one (60.0 mg, 0.100 mmol) and potassium fluoride (57.8 mg, 1.00 mmol) in THF (0.50 mL), DMF (0.50 mL) and water (50 μL), stirred for 1 hour at room temperature. The precipitated solids were collected by filtration and washed with THF/water (2:1) (3×3 mL) to yield the title compound as a yellow solid (13.0 mg, 29%).

(ES, m/z) [M+H]⁺=447.15

¹H-NMR (400 MHz, DMSO-d₆) δ 10.19 (s, 1H), 8.78 (s, 1H), 8.18 (d, J=2.4 Hz, 1H), 6.81 (d, J=8.5 Hz, 1H), 6.69 (dd, J=8.4, 2.3 Hz, 1H), 6.55 (s, 1H), 5.07 (s, 1H), 4.30 (dt, J=20.2, 5.7 Hz, 4H), 3.75 (q, J=6.4, 5.6 Hz, 4H), 2.93 (s, 4H), 2.44 (s, 4H), 2.21 (s, 3H).

### Example 233

[(1R,3S)-3-(Hydroxymethyl)cyclopentyl]methanol (16 g, 84.51%) [(1R,3S)-3-(hydroxymethyl)cyclopentyl]methanol

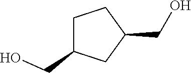

A solution of (1R,3S)-cyclopentane-1,3-dicarboxylic acid (23.0 g, 145 mmol, 1.0 eq.) in THF (230 mL) was treated with borane-THF complex (56.2 g, 654 mmol, 4.5 eq.) at 0° C. The resulting mixture was stirred for 14 hours at room temperature. The reaction was quenched with water (1.0 mL) at 0° C. then extracted with ethyl acetate (3×200 mL). The combined organic layers were washed with brine (2×100 mL), dried (Na₂SO₄) and concentrated under reduced pressure to yield the title compound as a yellow oil (16.0 g, 84%). [(1S,3R)-3-([(tert-Butyldiphenylsilyl)oxy]methylcyclopentyl]methanol

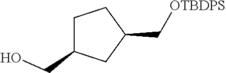

To a stirred solution of [(1R,3S)-3-(hydroxymethyl)cyclopentyl]methanol (10.0 g, 76.8 mmol, 1.0 eq.) in DMF (100 mL) was added imidazole (1.04 g, 154 mmol, 2.0 eq.) followed by tert-butyldiphenylsilyl chloride (21.1 g, 76.8 mmol, 1.0 eq.). The resulting mixture was stirred for 14 hours at room temperature. The resulting mixture was extracted with ethyl acetate (2×20 mL). The combined organic layers were washed with brine (3×30 mL), dried (Na₂SO₄) and concentrated under reduced pressure. The residue was purified by flash column chromatography eluting with ethyl acetate (7%) in 40-60 petroleum ether to afford the title compound as a light yellow oil (10.0 g, 35%).

### 1-(2-([(1S,3R)-3-([(tert-Butyldiphenylsilyl)oxy]methylcyclopentyl]methoxy-4-nitrophenyl)-4-methylpiperazine

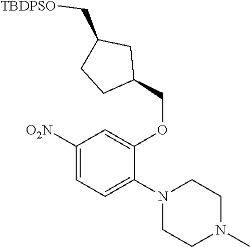

To a stirred solution of [(1S,3R)-3-([(tert-butyldiphenylsilyl)oxy]methylcyclopentyl]methanol (5.00 g, 13.6 mmol, 1.0 eq.) in dichloromethane (50 mL) w at 0° C. as added 2-(4-methylpiperazin-1-yl)-5-nitrophenol (3.86 g, 16.3 mmol, 1.2 eq.) followed by triphenylphosphine (7.12 g, 27.1 mmol, 2.0 eq.) and DIAD (5.49 g, 27.1 mmol, 2.0 eq.). The resulting mixture was stirred for 4 hours at room temperature. The residue was purified by reverse phase flash column chromatography eluting with acetonitrile (50-100%) in water (0.1% trifluoroacetic acid) to yield the title compound as a yellow solid (4.00 g, 50%).

### 3-([(1S,3R)-3-([(tert-Butyldiphenylsilyl)oxy]methylcyclopentyl]methoxy-4-(4-methylpiperazin-1-yl)aniline

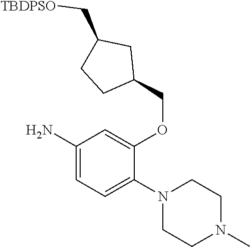

General procedure 12 was applied to 1-(2-([(1S,3R)-3-([(tert-butyldiphenylsilyl)oxy]methylcyclopentyl]methoxy-4-nitrophenyl)-4-methylpiperazine (2.50 g, 4.25 mmol) and palladium on carbon (498 mg, 4.68 mmol) in THF (30 mL). The mixture was hydrogenated at room temperature under 30 psi of hydrogen pressure to yield the title compound as a light yellow solid (2.00 g, 84%).

### 2-[(3-([(1S,3R)-3-([(tert-Butyldiphenylsilyl)oxy]methylcyclopentyl]methoxy-4-(4-methylpiperazin-1-yl)phenyl)amino]-5-[2-(triisopropylsilyl)ethynyl]-8H-pyrido[2,3-d]pyrimidin-7-one

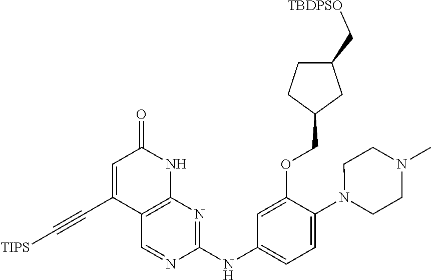

General procedure 13 was applied to 3-([(1S,3R)-3-([(tert-butyldiphenylsilyl)oxy]methylcyclopentyl]methoxy-4-(4-methylpiperazin-1-yl)aniline (2.00 g, 3.59 mmol), 2-methanesulfonyl-5-[2-(triisopropylsilyl)ethynyl]-8H-pyrido[2,3-d]pyrimidin-7-one (1.74 g, 4.30 mmol) and trifluoroacetic acid (472 mg, 4.14 mmol) in 2-methyl-2-butanol (10 mL), stirred for 14 hours at 100° C. The residue was purified by flash column chromatography eluting with methanol (10%) in dichloromethane to afford the title compound as a yellow solid (1.00 g, 31%).

### 2-[(3-{[(1S,3R)-3-(Hydroxymethyl)cyclopentyl]methoxy}-4-(4-methylpiperazin-1-yl)phen yl)amino]-5-[2-(triisopropylsilyl)ethynyl]-8H-pyrido[2,3-d]pyrimidin-7-one

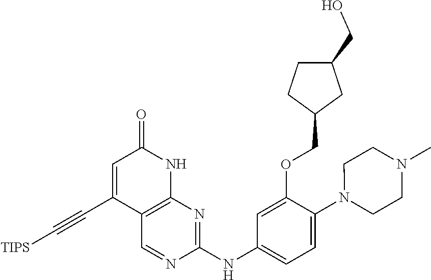

A solution of 2-[(3-{[(1S,3R)-3-{[(tert-butyldiphenylsilyl)oxy]methyl}cyclopentyl]methoxy}-4-(4-methylpiperazin-1-yl)phenyl)amino]-5-[2-(triisopropylsilyl)ethynyl]-8H-pyrido[2,3-d]pyrimidin-7-one (2.00 g, 2.26 mmol, 1.0 eq.) in hydrogen chloride in methanol (20 mL) was stirred for 14 hours at room temperature. The residue was purified by reverse phase flash column chromatography eluting with acetonitrile (50-100%) in water (0.1% trifluoroacetic acid) to yield the title compound as a yellow solid (500 mg, 34%).

### (5¹S,5³R)-1⁴-(4-Methylpiperazin-1-yl)-3⁵-((triisopropylsilyl)ethynyl)-3⁷,3⁸-dihydro-7-oxa-2-aza-3(2,8)-pyrido[2,3-d]pyrimidina-1(1,3)-benzena-5(1,3)-cyclopentanacycloheptaphan-3⁷-one

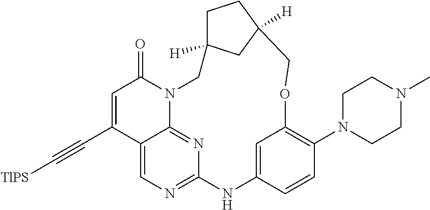

General procedure 20 was applied to 2-[(3-([(1S,3R)-3-(hydroxymethyl)cyclopentyl]methoxy-4-(4-methylpiperazin-1-yl)phenyl)amino]-5-[2-(triisopropylsilyl)ethynyl]-8H-pyrido[2,3-d]pyrimidin-7-one (300 mg, 0.465 mmol), triphenylphosphine (488 mg, 1.86 mmol) and DIAD (376 mg, 1.86 mmol) in THF (5.0 mL). The residue was purified by reverse phase flash column chromatography eluting acetonitrile (10-50%) in water (0.1% formic acid) to yield the title compound as an orange solid (50.0 mg, 17%).

### (5¹S,5³R)-3⁵-Ethynyl-1⁴-(4-methylpiperazin-1-yl)-3⁷,3⁸-dihydro-7-oxa-2-aza-3(2,8)-pyrido[2,3-d]pyrimidina-1(1,3)-benzena-5(1,3)-cyclopentanacycloheptaphan-3⁷-one

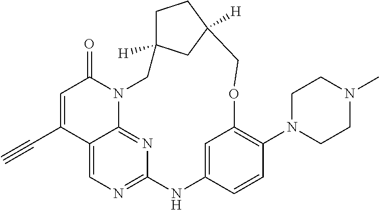 [SMILES: C#Cc1cc(=O)n2c3nc(ncc13)Nc1ccc(N3CCN(C)CC3)c(c1)OC[C@@H]1CC[C@@H](C1)C2]

General procedure 15 was applied to (5¹S,5³R)-1⁴-(4-Methylpiperazin-1-yl)-3⁵-((triisopropylsilyl)ethynyl)-3⁷,3⁸-dihydro-7-oxa-2-aza-3(2,8)-pyrido[2,3-d]pyrimidina-1(1,3)-benzena-5(1,3)-cyclopentanacycloheptaphan-3⁷-one (50.0 mg, 80.0 μmol) and potassium fluoride (46.3 mg, 0.800 mmol) in THF (2.0 mL), DMF (2.0 mL) and water (50 μL), stirred for 2 hours at room temperature. The resulting mixture was extracted with ethyl acetate (2×5.0 mL). The combined organic layers were washed with water (2×5 mL), dried (Na₂SO₄). The residue was washed with ethyl acetate (2×2.0 mL). After filtration, the filtrate was concentrated under reduced pressure to yield the title compound as a yellow solid (8.90 mg, 22%).

(ES, m/z) [M+H]⁺=471.10

¹H-NMR (400 MHz, DMSO-d₆) δ 10.27 (s, 1H), 8.77 (s, 1H), 8.55 (s, 1H), 6.81 (d, J=8.5 Hz, 1H), 6.74 (dd, J=8.6, 2.1 Hz, 1H), 6.55 (s, 1H), 5.06 (s, 1H), 4.35-3.89 (m, 4H), 2.95 (s, 4H), 2.45 (s, 4H), 2.21 (s, 3H), 1.86 (s, 2H), 1.61 (s, 4H).

### Example 234

### 3-((tert-Butyldiphenylsilyl)oxy)propan-1-amine

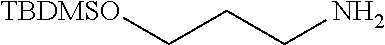 [SMILES: CC(C)(C)[Si](C)(C)OCCCN]

General procedure 21 was applied to 3-amino-1-propanol (3.00 g, 39.9 mmol), tert-butyldiphenylsilyl chloride (12.3 mL, 47.9 mmol) and imidazole (3.26 g, 47.9 mmol) in dichloromethane (150 mL). The crude product was purified by flash column chromatography eluting with methanol (5%) in dichloromethane to afford the title compound as a colourless oil (8.61 g, 69%).

¹H NMR (500 MHz, MeOD) δ 7.65-7.59 (4H, m), 7.52-7.41 (6H, m), 3.72 (2H, t, J=6.2 Hz), 2.83-2.77 (2H, m), 1.80-1.71 (2H, m), 1.00 (9H, s).

### N-(3-((tert-Butyidiphenylsilyl)oxy)propyl)-2-fluoro-5-nitrobenzenesulfonamide

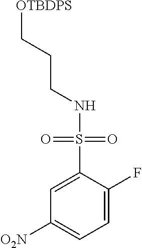

To a solution of 2-fluoro-5-nitrobenzene sulfonyl chloride (3.00 g, 12.5 mmol), triethylamine (2.10 mL, 15.0) in dichloromethane (20 mL), was added 3-((tert-butyldiphenylsilyl)oxy)propan-1-amine (4.71 g, 15.0 mmol) and left to stir at room temperature for 18 hours. The reaction mixture was diluted in ethyl acetate (30 mL), washed with water (20 mL), brine (20 mL), dried (MgSO₄) and concentrated under reduced pressure. The crude product was purified by flash column chromatography eluting ethyl acetate (20%) in petroleum ether to afford the title compound as a colourless oil (2.84 g, 44%).

¹H NMR (500 MHz, CDCl₃) δ 8.81-8.75 (1H, m), 8.46-8.40 (1H, m), 7.71-7.64 (4H, m), 7.46-7.34 (7H, m), 3.77 (2H, t, J=5.7 Hz), 3.35-3.28 (2H, m), 1.84-1.76 (2H, m), 1.07 (9H, s).

### N-(3-Hydroxypropyl)-2-(4-methylpiperazin-1-yl)-5-nitrobenzenesulfonamide

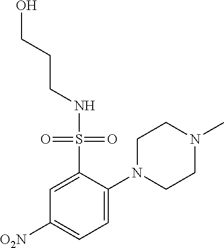 [SMILES: CN1CCN(c2ccc([N+](=O)[O-])cc2S(=O)(=O)NCCCO)CC1]

General procedure 11 was applied to N-(3-((tert-butyldiphenylsilyl)oxy)propyl)-2-fluoro-5-nitrobenzenesulfonamide (2.84 g, 5.50 mmol), N-methylpiperazine (1.84 mL, 16.5 mmol), potassium carbonate (1.14 g, 8.24 mmol) in acetonitrile (11 mL). The crude product was purified by flash column chromatography eluting with methanol (10%) in dichloromethane to afford the title compound as a yellow oil (1.35 g, 68%).

### N-(3-((tert-Butyldiphenylsilyl)oxy)propyl)-2-(4-methylpiperazin-1-yl)-5-nitrobenzenesulfonamide

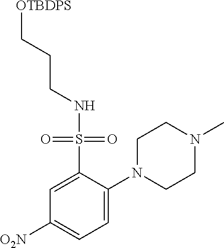

General procedure 21 was applied to N-(3-hydroxypropyl)-2-(4-methylpiperazin-1-yl)-5-nitrobenzenesulfonamide (1.35 g, 3.77 mmol), tert-butyldiphenylsilyl chloride (1.97 mL, 7.53 mmol), DMAP (46 mg, 0.377 mmol), triethylamine (1.06 mL, 7.63 mmol) in dichloromethane (20 mL). The crude product was purified by flash column chromatography eluting with methanol (5%) in dichloromethane to afford the title compound as a yellow oil (2.04 g, 91%).

¹H NMR (500 MHz, MeOD) δ 8.75 (1H, d, J=2.8 Hz), 8.39 (1H, d, J=8.9 and 2.8 Hz), 7.60-7.54 (5H, m), 7.43-7.34 (6H, m), 3.64 (1H, t, J=5.9 Hz), 3.21-3.16 (4H, m), 3.01-2.95 (2H, m), 2.67-2.62 (4H, m), 2.33 (3H, s), 1.69-1.60 (2H, m), 0.95 (9H, s).

### 5-Amino-N-(3-((tert-butyldiphenylsilyl)oxy)propyl)-2-(4-methylpiperazin-1-yl)benzenesulfonamide

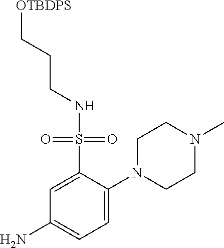

General procedure 12 was applied to 1-(2-(2-cyclopentylethoxy)-4-nitrophenyl)-4-methylpiperazine (2.04 g, 3.60 mmol) and palladium on carbon (204 mg, 1.92 mmol) in ethanol (50 mL) was stirred for 3 hours at room temperature under a hydrogen atmosphere. The resulting mixture was filtered, washing with ethanol (3×2.0 mL). The filtrate was concentrated under reduced pressure to afford the title compound as a brown oil (1.83 g, 90%).

¹H NMR (500 MHz, MeOD) δ 7.63-6.56 (4H, m), 7.44-7.34 (6H, m), 7.30-7.23 (2H, m), 6.92 (1H, dd, J=8.6 and 2.8 Hz), 3.69 (2H, t, J=5.8 Hz), 3.00-2.89 (6H, m), 2.69-2.63 (4H, m), 2.28 (3H, s), 1.72-1.63 (2H, m), 1.01 (9H, s).

### N-(3-Hydroxypropyl)-2-(4-methylpiperazin-1-yl)-5-((7-oxo-5-((triisopropylsilyl)ethynyl)-7,8-dihydropyrido[2,3-d]pyrimidin-2-yl)amino)benzenesulfonamide

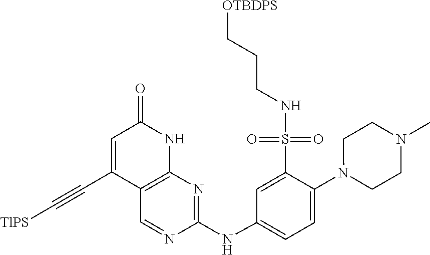

General procedure 13 was applied to 5-Amino-N-(3-((tert-butyldiphenylsilyl)oxy)propyl)-2-(4-methylpiperazin-1-yl)benzenesulfonamide (1.83 g, 3.23 mmol) with 2-(methylsulfonyl)-5-((triisopropylsilyl)ethynyl)pyrido[2,3-d]pyrimidin-7(8H)-one (1.19 g, 2.94 mmol) and trifluoroacetic acid (269 μL, 3.52 mmol) in acetonitrile (29 mL). The crude material was purified by flash column chromatography eluting with methanol (10%) in dichloromethane to yield the title compound as an orange solid (968 mg, 50%).

### 3⁴-(4-Methylpiperazin-1-yl)-1⁵-((triisopropylsilyl)ethynyl)-1⁷,1⁸-dihydro-4-thia-2,5-diaza-1(2,8)-pyrido[2,3-d]pyrimidina-3(1,3)-benzenacyclooctaphan-1⁷-one 4,4-dioxide

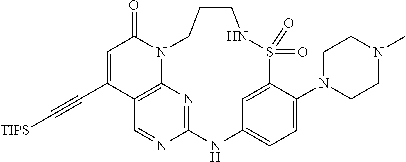

General procedure 20 was applied to N-(3-hydroxypropyl)-2-(4-methylpiperazin-1-yl)-5-((7-oxo-5-((triisopropylsilyl)ethynyl)-7,8-dihydropyrido[2,3-d]pyrimidin-2-yl)amino)benzenesulfonamide (968 mg, 1.48 mmol), DIAD (1.16 mL, 5.92 mmol) and triphenylphosphine (1.55 g, 5.92 mmol) in THF (61 mL). The crude product was purified by flash column chromatography eluting with methanol (10%) in dichloromethane to afford the title compound as a yellow solid (145 mg, 15%).

### 1⁵-Ethynyl-3⁴-(4-methylpiperazin-1-yl)-1⁷,1⁸-dihydro-4-thia-2,5-diaza-1(2,8)-pyrido[2,3-d]pyrimidina-3(1,3)-benzenacyclooctaphan-1⁷-one 4,4-dioxide

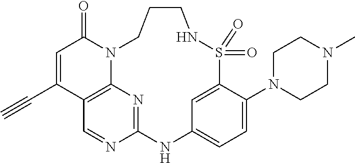 [SMILES: C#Cc1cc(=O)n2c3nc(ncc13)Nc1ccc(N3CCN(C)CC3)c(c1)S(=O)(=O)NCCC2]

General procedure 15 was applied to 3⁴-(4-methylpiperazin-1-yl)-15-((triisopropylsilyl)ethynyl)-1⁷,1⁸-dihydro-4-thia-2,5-diaza-1(2,8)-pyrido[2,3-d]pyrimidina-3(1.3)-benzenacyclooctaphan-1⁷-one 4,4-dioxide (45.0 mg, 0.231 mmol) with potassium fluoride (13.0 mg, 0.462 mmol) in DMF (1.0 mL). The crude material was purified by flash chromatography eluting with methanol (10%) in dichloromethane to yield the title compound as a yellow solid (8.00 mg, 7%).

(ES, m/z): [M+1]+=480

¹H NMR (500 MHz, DMSO-d₆) δ 10.65 (1H, s), 9.59 (1H, d, J=2.5 Hz), 8.83 (1H, s), 7.85 (1H, t, J=6.3 Hz), 7.51-7.43 (2H, m), 6.63 (1H, s), 4.40-4.34 (2H, m), 3.60 (1H, s), 3.47-3.43 (2H, m), 3.09-3.06 (4H, m), 2.94-2.91 (2H, m), 2.63-2.57 (4H, m), 2.60 (3H, s).

### Example 235

### 4-Hydroxy-N-[2-(4-isopropylpiperazin-1-yl)-5-nitrophenyl]butanamide

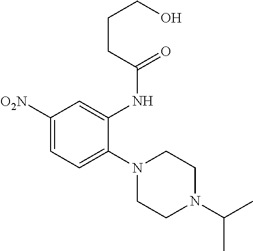 [SMILES: CC(C)N1CCN(c2ccc([N+](=O)[O-])cc2NC(=O)CCCO)CC1]

General procedure 11 was applied to N-(2-fluoro-5-nitrophenyl)-4-hydroxybutanamide (18.0 g, 74.3 mmol), 1-isopropyl-piperazine (11.4 g, 89.2 mmol) and potassium carbonate (20.5 g, 148 mmol) in acetonitrile (180 mL) was stirred overnight at 80° C. The residue was purified by flash column chromatography eluting with methanol (20%) in dichloromethane to afford the title compound as a yellow oil (16.0 g, 61%).

### 4-[(tert-Butyldiphenylsilyl)oxy]-N-[2-(4-isopropylpiperazin-1-yl)-5-nitrophenyl]butanamide

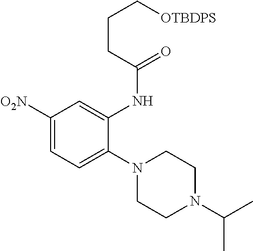

General procedure 21 was applied to 4-hydroxy-N-[2-(4-isopropylpiperazin-1-yl)-5-nitrophenyl]butanamide (3.00 g, 8.56 mmol), tert-butyl(chloro)diphenylsilane (4.24 g, 15.4 mmol) and imidazole (1.75 g, 25.7 mmol) in DMF (10 mL). The residue was purified by reverse-phase flash column chromatography eluting acetonitrile (30-90%) in water (0.1% ammonium carbonate) to afford the title compound as a yellow oil (4.59 g, 89%).

### N-[5-Amino-2-(4-isopropylpiperazin-1-yl)phenyl]-4-[(tert-butyldiphenylsilyl)oxy]butanamide

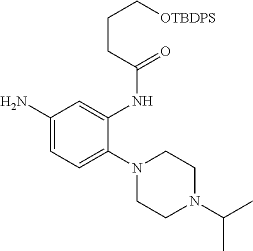

General procedure 12 was applied to 4-[(tert-butyldiphenylsilyl)oxy]-N-[2-(4-isopropylpiperazin-1-yl)-5-nitrophenyl]butanamide (4.50 g, 7.64 mmol) and palladium on carbon (900 mg) in methanol (45 mL) to afford the title compound as a yellow solid (3.50 g, 82%). The crude product was used in the next step directly without further purification.

### 4-[(tert-Butyldiphenylsilyl)oxy]-N-[2-(4-isopropylpiperazin-1-yl)-5-({7-oxo-5-[2-(triisopropylsilyl)ethynyl]-8H-pyrido[2,3-d]pyrimidin-2-yl}amino)phenyl]butanamide

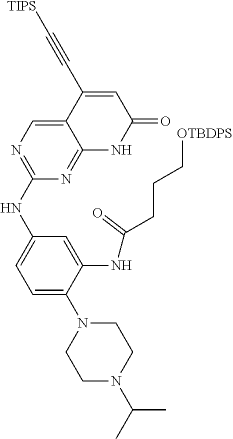

General procedure 13 was applied to N-[5-amino-2-(4-isopropylpiperazin-1-yl)phenyl]-4-[(tert-butyldiphenylsilyl)oxy]butanamide (2.81 g, 5.03 mmol), 2-methanesulfonyl-5-[2-(triisopropylsilyl)ethynyl]-8H-pyrido[2,3-d]pyrimidin-7-one (1.70 g, 4.19 mmol) and trifluoroacetic acid (960 mg, 8.43 mmol) in butan-2-ol (30 mL), stirred overnight at 100° C. The residue was purified by reverse-phase flash column chromatography eluting with methanol (20-100%) in water (0.1% trifluoroacetic acid) to afford the title compound as a yellow solid (2.20 g, 59%).

### 4-Hydroxy-N-[2-(4-isopropylpiperazin-1-yl)-5-({7-oxo-5-[2-(triisopropylsilyl)ethynyl]-8H-pyrido[2,3-d]pyrimidin-2-yl}amino)phenyl]butanamide

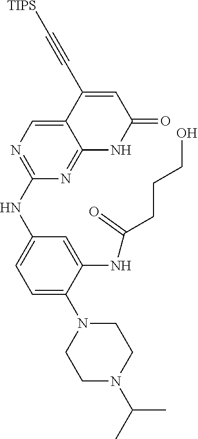

A mixture of 4-[(tert-butyldiphenylsilyl)oxy]-N-[2-(4-isopropylpiperazin-1-yl)-5-({7-oxo-5-[2-(triisopropylsilyl)ethynyl]-8H-pyrido[2,3-d]pyrimidin-2-yl}amino)phenyl]butanamide (1.00 g, 1.13 mmol, 1.0 eq.) and hydrogen chloride (1 M, 10 mL) in THF (20 mL) was stirred overnight at room temperature. The residue was purified by reverse-phase flash column chromatography eluting with acetonitrile (10-60%) in water (0.1% trifluoroacetic acid) to afford the title compound as a yellow solid (300 mg, 41%).

### 3⁴-(4-Isopropylpiperazin-1-yl)-1⁵-((triisopropylsilyl)ethynyl)-1⁷,1⁸-dihydro-2,4-diaza-1(2, 8)-pyrido[2,3-d]pyrimidina-3(1,3)-benzenacyclooctaphane-1⁷,5-dione

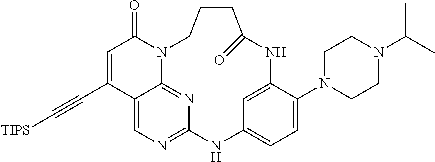

General procedure 20 was applied to 4-hydroxy-N-[2-(4-isopropylpiperazin-1-yl)-5-({7-oxo-5-[2-(triisopropylsilyl)ethynyl]-8H-pyrido[2,3-d]pyrimidin-2-yl}amino)phenyl]butanamide (280 mg, 0.433 mmol), triphenylphosphine (455 mg, 1.73 mmol) and DEAD (302 mg, 1.73 mmol) in THF (6 mL). The residue was purified by reverse-phase flash column chromatography eluting with acetonitrile (10-70%) in water (0.1% trifluoroacetic acid), to afford the title compound as a yellow solid (120 mg, 44%).

### 1⁵-Ethynyl-3⁴-(4-isopropylpiperazin-1-yl)-1⁷,1⁸-dihydro-2,4-diaza-1(2,8)-pyrido[2,3-d]pyrimidina-3(1,3)-benzenacyclooctaphane-1⁷,5-dione

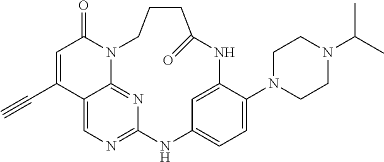 [SMILES: C#Cc1cc(=O)n2c3nc(ncc13)Nc1ccc(N3CCN(C(C)C)CC3)c(c1)NC(=O)CCC2]

General procedure 15 was applied to 3⁴-(4-Isopropylpiperazin-1-yl)-1⁵-((triisopropylsilyl)ethynyl)-1⁷,1⁸-dihydro-2,4-diaza-1(2,8)-pyrido[2,3-d]pyrimidina-3(1,3)-benzenacyclooctaphane-1⁷,5-dione (100 mg, 0.159 mmol) and potassium fluoride (92.5 mg, 1.59 mmol) in DMF (0.50 mL), THF (0.50 mL) and water (50 μL), stirred for 1 hour at room temperature. The crude product was purified by Prep-HPLC eluting with acetonitrile (20-40%) in water (0.1% ammonium carbonate, 0.05% ammonium hydroxide) to afford the title compound as a yellow solid (3.00 mg, 4%).

(ES, m/z): [M+H]⁺=472.10

¹H-NMR (400 MHz, DMSO-d₆) δ 10.18 (s, 1H), 8.75 (s, 1H), 8.66 (s, 1H), 7.73 (d, J=2.3 Hz, 1H), 7.05-6.92 (m, 2H), 6.52 (s, 1H), 4.98 (s, 1H), 2.84 (s, 4H), 2.69-2.63 (m, 1H), 3.28-3.17 (m, 2H), 2.57 (s, 4H), 2.28 (d, J=8.9 Hz, 2H), 2.01 (s, 2H), 1.00 (d, J=6.5 Hz, 6H).

### Example 236

### 4-Hydroxy-N-{2-[4-(2-methoxyethyl)piperazin-1-yl]-5-nitrophenyl}butanamide

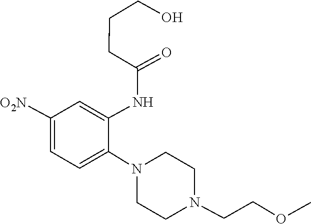 [SMILES: COCCN1CCN(c2ccc([N+](=O)[O-])cc2NC(=O)CCCO)CC1]

General procedure 11 was applied to N-(2-fluoro-5-nitrophenyl)-4-hydroxybutanamide (18.0 g, 74.3 mmol), 1-(2-methoxyethyl)piperazine (12.8 g, 89.2 mmol) and potassium carbonate (20.5 g, 149 mmol) in acetonitrile (180 mL) m stirred overnight at 80° C. The residue was purified by flash column chromatography eluting with methanol (20%) in dichloromethane to afford the title compound as a yellow oil (17.8 g, 65%).

### 4-[(tert-Butyldiphenylsilyl)oxy]-N-{2-[4-(2-methoxyethyl)piperazin-1-yl]-5-nitrophenyl}butanamide

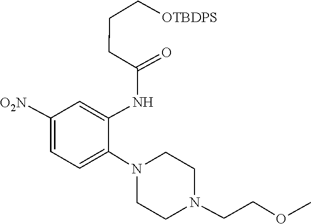

General procedure 21 was applied to 4-hydroxy-N-{2-[4-(2-methoxyethyl)piperazin-1-yl]-5-nitrophenyl}butanamide (3.50 g, 9.55 mmol), tert-butyl(chloro)diphenylsilane (4.73 g, 17.2 mmol) and imidazole (1.95 g, 28.6 mmol) in DMF (15 mL). The residue was purified by reverse-phase flash column chromatography eluting with acetonitrile (30-90%) in water (0.1% ammonium carbonate) to yield the title compound as a yellow oil (4.40 g, 76%).

### N-{5-Amino-2-[4-(2-methoxyethyl)piperazin-1-yl]phenyl}-4-[(tert-butyldiphenylsilyl)oxy]butanamide

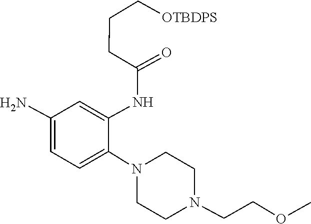

General procedure 12 was applied to 4-[(tert-butyldiphenylsilyl)oxy]-N-{2-[4-(2-methoxyethyl)piperazin-1-yl]-5-nitrophenyl}butanamide (4.40 g, 7.27 mmol) and palladium on carbon (880 mg) in methanol (44 mL) to yield the title compound as a white solid (3.80 g, 91%). The crude product was used in the next step directly without further purification. 4-((tert-Butyldiphenylsilyl)oxy)-N-(2-(4-(2-methoxyethyl)piperazin-1-yl)-5-((7-oxo-5-((triisopropylsilyl)ethynyl)-7,8-dihydropyrido[2,3-d]pyrimidin-2-yl)amino)phenyl)butanamide

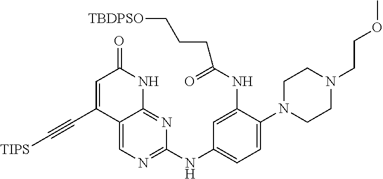

General procedure 13 was applied to N-{5-amino-2-[4-(2-methoxyethyl)piperazin-1-yl]phenyl}-4-[(tert-butyldiphenylsilyl)oxy]butanamide (3.01 g, 5.24 mmol) and 2-methanesulfonyl-5-[2-(triisopropylsilyl)ethynyl]-8H-pyrido[2,3-d]pyrimidin-7-one (1.77 g, 4.36 mmol) and trifluoroacetic acid (1.00 g, 8.73 mmol) in butan-2-ol (30 mL), stirred overnight at 100° C. The residue was purified by reverse-phase flash column chromatography eluting with methanol (20-100%) in water (0.1% trifluoroacetic acid) to yield the title compound as a yellow solid (2.70 g, 69%).

### 4-Hydroxy-N-{2-[4-(2-methoxyethyl)piperazin-1-yl]-5-({7-oxo-5-[2-(triisopropylsilyl)ethynyl]-8H-pyrido[2,3-d]pyrimidin-2-yl}amino)phenyl}butanamide

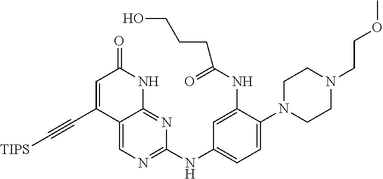

A mixture of 4-[(tert-butyldiphenylsilyl)oxy]-N-{2-[4-(2-methoxyethyl)piperazin-1-yl]-5-({7-oxo-5-[2-(triisopropylsilyl)ethynyl]-8H-pyrido[2,3-d]pyrimidin-2-yl}amino)phenyl}butanamide (1.00 g, 1.11 mmol, 1.0 eq.) and hydrogen chloride (1 M, 10 mL) in THF (20 mL) was stirred overnight at room temperature. The residue was purified by reverse-phase flash column chromatography eluting with acetonitrile (10-60%) in water (0.1% trifluoroacetic acid), to afford the title compound as a yellow solid (180 mg, 24%).

### 3⁴-(4-(2-Methoxyethyl)piperazin-1-yl)-1⁵-((triisopropylsilyl)ethynyl)-1⁷,1⁸-dihydro-2,4-diaza-1(2,8)-pyrido[2,3-d]pyrimidina-3(1,3)-benzenacyclooctaphane-1⁷,5-dione

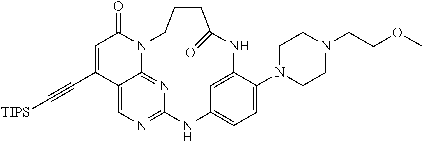

General procedure 20 was applied to 4-hydroxy-N-{2-[4-(2-methoxyethyl)piperazin-1-yl]-5-({7-oxo-5-[2-(triisopropylsilyl)ethynyl]-8H-pyrido[2,3-d]pyrimidin-2-yl}amino)phenyl}butanamide (170 mg, 0.257 mmol), triphenylphosphine (539 mg, 2.06 mmol) and DEAD (415 mg, 2.06 mmol) in THF (10 mL). The residue was purified by reverse-phase flash column chromatography eluting with acetonitrile (10-70%) in water (0.1% trifluoroacetic acid) to afford the title compound as a yellow solid (70.0 mg, 42%).

### 1⁵-Ethynyl-3⁴-(4-(2-methoxyethyl)piperazin-1-yl)-1⁷,1⁸-dihydro-2,4-diaza-1(2,8)-pyrido[2, 3-d]pyrimidina-3(1,3)-benzenacyclooctaphane-1⁷,5-dione

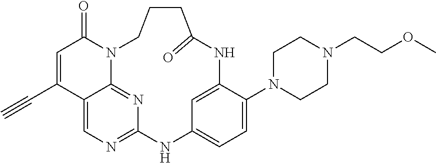 [SMILES: C#Cc1cc(=O)n2c3nc(ncc13)Nc1ccc(N3CCN(CCOC)CC3)c(c1)NC(=O)CCC2]

General procedure 15 was applied to 3⁴-(4-(2-Methoxyethyl)piperazin-1-yl)-15-((triisopropylsilyl)ethynyl)-1⁷,1⁸-dihydro-2,4-diaza-1(2,8)-pyrido[2,3-d]pyrimidina-3(1,3)-benzenacyclooctaphane-1⁷,5-dione (70.0 mg, 0.109 mmol) and potassium fluoride (67.7 mg, 1.16 mmol) in THF (2.0 mL) and water (70 μL), stirred for 4 hours at room temperature. The crude material was washed with ethyl acetate (2×3.0 mL) to yield the title compound as a yellow solid (5.80 mg, 10%).

(ES, m/z): [M+H]⁺=488.30

¹H-NMR (400 MHz, DMSO-d₆) δ 10.19 (s, 1H), 8.76 (s, 1H), 8.63 (s, 1H), 7.74 (d, J=2.3 Hz, 1H), 7.05-6.92 (m, 2H), 6.52 (s, 1H), 5.04 (s, 1H), 3.93 (s, 2H), 3.46 (t, J=5.8 Hz, 2H), 3.25 (s, 3H), 2.87 (d, J=16.3 Hz, 4H), 2.55 (d, J=15.4 Hz, 6H), 2.28 (d, J=9.8 Hz, 2H), 2.00 (d, J=10.0 Hz, 2H).

### Example 237

### N-(2-Fluoro-5-nitrobenzyl)-4-hydroxybutanamide

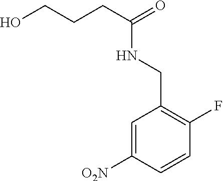 [SMILES: O=C(CCCO)NCc1cc([N+](=O)[O-])ccc1F]

To a solution of γ-butyrolactone (5.00 g, 58.1 mmol) in THF (15 mL) and water (15 mL) was added lithium hydroxide (1.53 g, 63.9 mmol) and left to stir at room temperature for 3 hours. The reaction mixture was concentrated under reduced pressure to give a crude material. The crude material was redissolved in DMF (50 mL) along with (2-fluoro-5-nitrophenyl)methanamine hydrogen chloride (5.00 g, 24.2 mmol), diisopropylethylamine (8.46 mL, 48.8 mmol), EDC (5.56 g, 29.0 mmol) and HOBT (3.92 g, 29.0 mmol) and left to stir at room temperature for 18 hours. The reaction mixture was diluted in ethyl acetate, washed with water (20 mL), brine (20 mL), dried (MgSO₄) and concentrated under reduced pressure. The crude product was purified by flash column chromatography eluting with methanol (10%) in dichloromethane to afford the title compound as a colourless oil (3.54 g, 57%).

¹H NMR (500 MHz, CDCl₃) δ 8.16-8.10 (1H, m), 8.08-8.00 (1H, m), 7.66-7.55 (1H, m), 7.16-7.07 (1H, m), 4.44-4.38 (2H, m), 4.15-3.96 (1H, br. s), 3.61-3.49 (2H, m), 2.40-2.27 (2H, m), 1.84-1.72 (2H, m).

### 4-Hydroxy-N-(2-(4-methylpiperazin-1-yl)-5-nitrobenzyl)butanamide

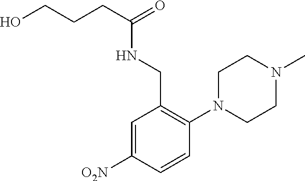 [SMILES: CN1CCN(c2ccc([N+](=O)[O-])cc2CNC(=O)CCCO)CC1]

General procedure 11 was applied to N-(2-Fluoro-5-nitrobenzyl)-4-hydroxybutanamide (3.54 g, 13.8 mmol), N-methylpiperazine (3.07 mL, 27.6 mmol), potassium carbonate (3.82 g, 27.6 mmol) in acetonitrile (35 mL). The crude product was purified by flash column chromatography eluting with methanol (10%) in dichloromethane to afford the title compound as a yellow oil (3.61 g, 78%).

¹H NMR (500 MHz, CDCl₃) δ 8.13 (1H, d, J=2.8 Hz), 8.07 (1H, dd, J=8.9 and 2.8 Hz), 7.20 (1H, d, J=8.9 Hz), 4.49 (2H, s), 3.67-3.57 (2H, m), 3.12-3.05 (4H, m), 2.89-2.83 (2H, m), 2.69-2.58 (4H, m), 2.48-2.40 (2H, m), 2.38 (3H, s), 1.97-1.85 (2H, m).

### 4-((tert-Butyldiphenylsilyl)oxy)-N-(2-(4-methylpiperazin-1-yl)-5-nitrobenzyl)butanamide

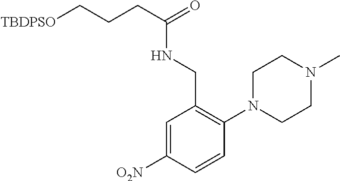

General procedure 21 was applied to 4-hydroxy-N-(2-(4-methylpiperazin-1-yl)-5-nitrobenzyl)butanamide (3.61 g, 10.7 mmol), tert-butyldiphenylsilyl chloride (5.60 mL, 21.5 mmol), DMAP (131 mg, 1.07 mmol), triethylamine (2.90 mL, 21.5 mmol) in dichloromethane (50 mL). The crude product was purified by flash column chromatography eluting with methanol (5%) in dichloromethane to afford the title compound as a yellow oil (4.30 g, 70%).

¹H NMR (500 MHz, MeOD) δ8.13 (1H, d, J=2.7 Hz), 8.05 (1H, dd, J=8.8 and 2.8 Hz), 7.71-7.62 (4H, m), 7.43-7.32 (6H, m), 7.16 (1H, d, J=8.8 Hz), 4.47 (2H, s), 3.77-3.70 (2H, m), 3.06-2.98 (4H, m), 2.63-2.59 (4H, m), 2.50-2.38 (2H, m), 2.34 (3H, s), 1.96 (2H, m), 1.04 (9H, s).

### N-(5-Amino-2-(4-methylpiperazin-1-yl)benzyl)-4-((tert-butyidiphenylsilyl)oxy)butanamide

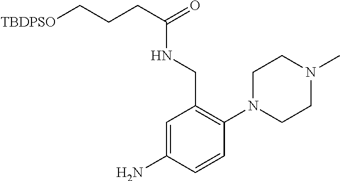

General procedure 12 was applied to 4-((tert-butyldiphenylsilyl)oxy)-N-(2-(4-methylpiperazin-1-yl)-5-nitrobenzyl)butanamide (4.30 g, 7.48 mmol) and palladium on carbon (430 mg, 4.04 mmol) in ethanol (50 mL) was stirred for 3 hours at room temperature under an hydrogen atmosphere. The resulting mixture was filtered, washing with ethanol (3×2.0 mL). The filtrate was concentrated under reduced pressure to afford the title compound as a brown oil (4.06 g, 99%).

¹H NMR (500 MHz, MeOD) δ 7.71-7.63 (4H, m), 7.47-7.34 (6H, m), 7.02 (1H, d, J=8.2 Hz), 6.72-6.65 (2H, m), 4.42 (2H, s), 3.73 (1H, t, J=6.3 Hz), 3.33-3.29 (4H, m), 3.02-2.99 (4H, m), 2.86 (3H, s), 2.46-2.37 (2H, m), 1.19 (2H, t, J=7.1 Hz), 0.95 (9H, s).

### 2-((3-(Aminomethyl)-4-(4-methylpiperazin-1-yl)phenyl)amino)-5-((triisopropylsilyl)ethynyl)pyrido[2,3-d]pyrimidin-7(8H)-one

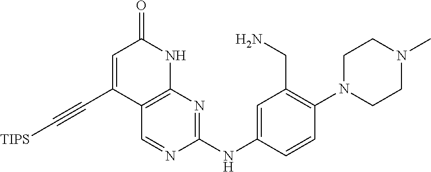

General procedure 13 was applied to N-(5-amino-2-(4-methylpiperazin-1-yl)benzyl)-4-((tert-butyldiphenylsilyl)oxy)butanamide (4.06 g, 7.45 mmol) with 2-(methylsulfonyl)-5-((triisopropylsilyl)ethynyl)pyrido[2,3-d]pyrimidin-7(8H)-one (2.75 g, 6.77 mmol) and trifluoroacetic acid (661 μL, 8.13 mmol) in acetonitrile (67 mL). The crude material was purified by flash column chromatography eluting with methanol (10%) in dichloromethane to yield the title compound as an orange solid (1.27 g, 34%).

¹H NMR (500 MHz, MeOD) δ 8.77 (1H, s), 8.01-7.93 (1H, m), 7.83-7.76 (1H, m), 7.32 (1H, d, J=8.2 Hz), 6.34 (1H, s), 4.27 (2H, s), 3.63-3.46 (4H, m), 3.32-3.21 (4H, m), 3.00 (3H, s).

### 1⁵-Ethynyl-3⁴-(4-methylpiperazin-1-yl)-1⁷,1⁸-dihydro-2,5-diaza-1(2,8)-pyrido[2,3-d]pyrimidina-3(1,3)-benzenacyclononaphane-1⁷,6-dione

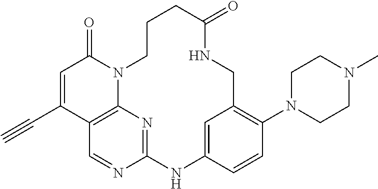 [SMILES: C#Cc1cc(=O)n2c3nc(ncc13)Nc1ccc(N3CCN(C)CC3)c(c1)CNC(=O)CCC2]

To a solution of γ-butyrolactone (5.00 g, 58.1 mmol) in THF (15 mL) and water (15 mL) was added lithium hydroxide (1.53 g, 63.9 mmol) and left to stir at room temperature for 3 hours. The reaction mixture was concentrated under reduced pressure to give a crude material. the crude material was redissolved in DMF (50 mL) along with 2-((3-(aminomethyl)-4-(4-methylpiperazin-1-yl)phenyl)amino)-5-((triisopropylsilyl)ethynyl)pyrido[2,3-d]pyrimidin-7(8H)-one (1.27 g, 2.53 mmol), diisopropylethylamine (610 μL, 3.49 mmol), EDC (535 mg, 2.79 mmol) and HOBT (427 mg, 2.79 mmol) and left to stir at room temperature for 18 hours. The reaction mixture was diluted in ethyl acetate, washed with water (20 mL), brine (20 mL), dried (MgSO₄) and concentrated under reduced pressure to give the crude product.

General procedure 20 was applied to the crude material (229 mg, 0.472 mmol), DIAD (371 μL, 1.89 mmol) and triphenylphosphine (496 mg, 1.89 mmol) in THF (20 mL). The crude material was purified by flash column chromatography eluting with methanol (10%) in dichloromethane to afford a crude product.

General procedure 15 was applied to the crude material (44.0 mg, 72.0 μmol) with potassium fluoride (21.0 mg, 0.360 mmol) in DMF (1.0 mL). The crude material was purified by flash chromatography eluting with methanol (10%) in dichloromethane to yield the title compound as a yellow solid (25.0 mg, 75%).

(ES, m/z): [M+1]+=458

¹H NMR (500 MHz, DMSO-d₆) δ 9.95 (1H, s), 8.47 (1H, s), 8.25-8.19 (1H, m), 7.99 (1H, d, J=2.4 Hz), 6.84-6.74 (2H, m), 6.25 (1H, s), 4.73 (1H, s), 4.07 (2H, d, J=6.7 Hz), 3.97-3.90 (2H, m), 2.53-2.47 (4H, m), 2.22-2.13 (4H, m), 2.01-1.95 (2H, m), 1.69-1.61 (2H, m).

### Example 238

### 4-Hydroxy-N-[2-(4-methyl-1,4-diazepan-1-yl)-5-nitrophenyl]butanamide

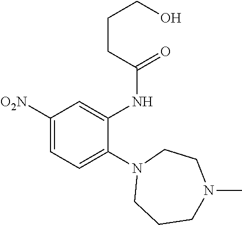 [SMILES: CN1CCCN(c2ccc([N+](=O)[O-])cc2NC(=O)CCCO)CC1]

General procedure 11 was applied to N-(2-fluoro-5-nitrophenyl)-4-hydroxybutanamide (10.0 g, 41.3 mmol), N-methylhomopiperazine (5.66 g, 49.5 mmol) and potassium carbonate (11.4 g, 82.6 mmol) in acetonitrile (100 mL), stirred overnight at 80° C. The residue was purified by flash column chromatography eluting with methanol (15%) in dichloromethane to afford the title compound as a yellow oil (7.80 g, 56%).

### 4-[(tert-Butyldiphenylsilyl)oxy]-N-[2-(4-methyl-1,4-diazepan-1-yl)-5-nitrophenyl]butanamide

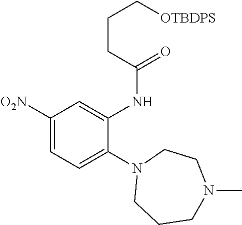

General procedure 21 was applied to 4-hydroxy-N-[2-(4-methyl-1,4-diazepan-1-yl)-5-nitrophenyl]butanamide (7.80 g, 23.2 mmol), tert-butyl(chloro)diphenylsilane (11.5 g, 41.7 mmol) and imidazole (4.74 g, 69.5 mmol) in DMF (40 mL). The residue was purified by reverse-phase flash column chromatography eluting with acetonitrile (30-90%) in water (0.1% ammonium carbonate) to afford the title compound as a yellow oil (12.0 g, 90%).

### N-[5-Amino-2-(4-methyl-1,4-diazepan-1-yl)phenyl]-4-[(tert-butyldiphenylsilyl)oxy]butanamide

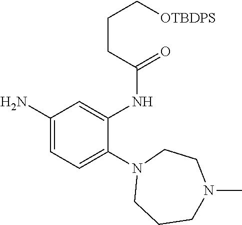

General procedure 12 was applied to 4-[(tert-butyldiphenylsilyl)oxy]-N-[2-(4-methyl-1,4-diazepan-1-yl)-5-nitrophenyl]butanamide (10.0 g, 17.4 mmol) and palladium on carbon (2.00 g, 20% wt) in methanol (100 mL) to yield the title compound as a yellow oil (9.30 g, 98%). The crude product was used in the next step directly without further purification.

### 4-[(tert-Butyldiphenylsilyl)oxy]-N-[2-(4-methyl-1,4-diazepan-1-yl)-5-({7-oxo-5-[2-(triisopropylsilyl)ethynyl]-8H-pyrido[2,3-d]pyrimidin-2-yl}amino)phenyl]butanamide

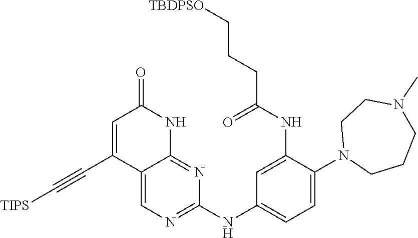

General procedure 13 was applied to N-[5-amino-2-(4-methyl-1,4-diazepan-1-yl)phenyl]-4-[(tert-butyldiphenylsilyl)oxy]butanamide (3.00 g, 5.50 mmol) and 2-methanesulfonyl-5-[2-(triisopropylsilyl)ethynyl]-8H-pyrido[2,3-d]pyrimidin-7-one (1.86 g, 4.58 mmol) and trifluoroacetic acid (1.05 g, 9.21 mmol) in butan-2-ol (30 mL), stirred overnight at 100° C. The residue was purified by reverse-phase flash column chromatography eluting with acetonitrile (20-100%) in water (0.1% trifluoroacetic acid) to yield the title compound as a yellow solid (2.10 g, 52%).

### 4-Hydroxy-N-[2-(4-methyl-1,4-diazepan-1-yl)-5-({7-oxo-5-[2-(triisopropyisiyl)ethynyl]-8H-pyrido[2,3-d]pyrimidin-2-yl}amino)phenyl]butanamide

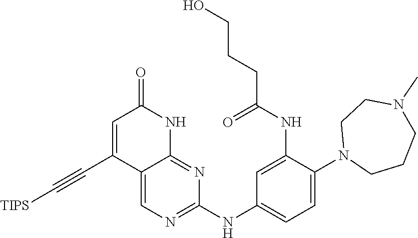

A mixture of 4-[(tert-butyldiphenylsilyl)oxy]-N-[2-(4-methyl-1,4-diazepan-1-yl)-5-({7-oxo-5-[2-(triisopropylsilyl)ethynyl]-8H-pyrido[2,3-d]pyrimidin-2-yl}amino)phenyl]butanamide (450 mg, 0.517 mmol, 1.0 eq.) and hydrogen chloride (aq. 1 M, 4.5 mL) in THF (9.0 mL) was stirred overnight at room temperature. The residue was purified by reverse-phase flash column chromatography eluting with methanol (30-100%) in water (0.1% ammonium carbonate), to yield the title compound as a yellow solid (300 mg, 92%).

### 3⁴-(4-Methyl-1,4-diazepan-1-yl)-1⁵-((triisopropylsilyl)ethynyl)-1⁷,1⁸-dihydro-2,4-diaza-1(2,8)-pyrido[2,3-d]pyrimidina-3(1,3)-benzenacyclooctaphane-1⁷,5-dione

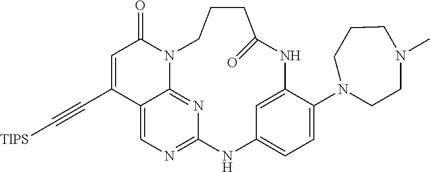

General procedure 20 was applied to 4-hydroxy-N-[2-(4-methyl-1,4-diazepan-1-yl)-5-({7-oxo-5-[2-(triisopropylsilyl)ethynyl]-8H-pyrido[2,3-d]pyrimidin-2-yl}amino)phenyl]butanamide (200 mg, 0.317 mmol), triphenylphosphine (332 mg, 1.27 mmol) and DEAD (220 mg, 1.27 mmol) in THF (10 mL). The residue was purified by reverse-phase flash column chromatography eluting acetonitrile (10-70%) in water (0.15 trifluoroacetic acid) to yield the title compound as a yellow solid (80.0 mg, 41%).

### 1⁵-Ethynyl-3⁴-(4-methyl-1,4-diazepan-1-yl)-1⁷,1⁸-dihydro-2,4-diaza-1(2,8)-pyrido[2,3-d]pyrimidina-3(1,3)-benzenacyclooctaphane-1⁷,5-dione

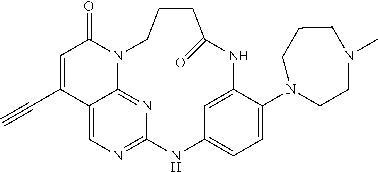 [SMILES: C#Cc1cc(=O)n2c3nc(ncc13)Nc1ccc(N3CCCN(C)CC3)c(c1)NC(=O)CCC2]

General procedure 15 was applied to 3⁴-(4-methyl-1,4-diazepan-1-yl)-15-((triisopropylsilyl)ethynyl)-1⁷,1⁸-dihydro-2,4-diaza-1(2,8)-pyrido[2,3-d]pyrimidina-3(1,3)-benzenacyclooctaphane-1⁷,5-dione (80.0 mg, 0.130 mmol) and potassium fluoride (75.7 mg, 1.30 mmol in THF (3.0 mL), DMF (3.0 mL) and water (80 μL), stirred for 2 hours at room temperature. The residue was washed with acetonitrile/water (1/1) (3×5.0 mL) to yield the title compound as a yellow solid (20.0 mg, 33%).

(ES, m/z): [M+H]⁺=458.25

¹H-NMR (400 MHz, DMSO-d₆) δ 10.12 (s, 1H), 8.75 (d, J=6.4 Hz, 2H), 7.63 (d, J=2.5 Hz, 1H), 7.00-6.90 (m, 2H), 6.51 (s, 1H), 5.03 (s, 1H), 4.08-3.55 (m, 2H), 3.18 (t, J=6.1 Hz, 4H), 2.61 (q, J=6.7, 5.3 Hz, 4H), 2.29 (s, 3H), 2.27-2.20 (m, 2H), 2.00 (d, J=8.8 Hz, 2H), 1.83 (p, J=5.9 Hz, 2H).

### Example 239

### Methyl (S)-2-((tert-butyidiphenylsilyl)oxy)propanoate

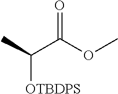

General procedure 21 was applied to (−)-methyl-L-lactate (5.00 g, 48.0 mmol), tert-butyldiphenylsilyl chloride (15.0 mL, 57.6 mmol) and imidazole (4.90 g, 72.0 mmol) in DMF (125 mL). The crude product was purified by flash column chromatography eluting ethyl acetate (25%) in petroleum ether to afford the title compound as a colourless oil (12.4 g, 75%).

¹H NMR (500 MHz, CDCl₃) δ 7.69-7.63 (4H, m), 7.47-7.32 (6H, m), 4.28 (1H, q, J=6.7 Hz), 3.56 (3H, s), 1.38 (3H, d, J=6.7 Hz), 1.07 (9H, s).

### (S)-2-((tert-Butyldiphenylsilyl)oxy)propan-1-ol

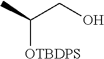

To a stirred solution of methyl (S)-2-((tert-butyldiphenylsilyl)oxy)propanoate (12.38 g, 36.2 mmol) in THF (27 mL) was added lithium borohydride in THF (2M, 27.0 mL, 54.2 mmol). The reaction mixture was stirred overnight under a nitrogen atmosphere. The reaction was quenched with water (20 mL) and extracted with ethyl acetate (3×20 mL), washed with brine, dried (MgSO₄) and concentrated under reduced pressure to give the title compound as a colourless oil (11.1 g, 100%). No further purification was required.

¹H NMR (500 MHz, CDCl₃) δ 7.75-7.64 (4H, m), 7.48-7.34 (6H, m), 4.00-3.92 (1H, m), 3.58-3.40 (2H, m), 1.13-1.02 (12H, m).

### (S)-2-((tert-Butyldiphenylsilyl)oxy)propanal

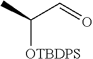

To a solution of (S)-2-((tert-butyldiphenylsilyl)oxy)propan-1-ol (11.1 g, 35.4 mmol) in dichloromethane (150 mL), was added DMP (18.0 g, 42.5 mmol) and left to stir at room temperature for 2 hours. The reaction mixture was washed with saturated aqueous sodium thiosulfate (20 mL), saturated aqueous sodium hydrogen carbonate (20 mL) and brine (20 mL), dried (MgSO₄) and concentrated under reduced pressure. The crude product was passed through a silica plug eluting with 100% petroleum ether to give the title compound as a colourless oil (2.05 g, 18%).

¹H NMR (500 MHz, CDCl₃) δ 9.64 (1H, d, J=1.2 Hz), 7.72-7.60 (4H, m), 7.48-7.27 (6H, m), 4.09 (1H, qd, J=6.9 and 1.2 Hz), 1.22 (3H, d, J=6.9 Hz), 1.11 (9H, s).

### Ethyl (S,E)-4-((tert-butyldiphenylsilyl)oxy)pent-2-enoate

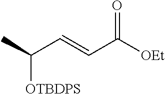

To a solution of triethyl phosphonoacetate (1.67 mL, 8.55 mmol) and lithium chloride (319 mg, 7.54 mmol) in acetonitrile (10 mL) was added DBU (1.03 mL, 6.91 mmol) and stirred at room temperature for 30 minutes. (S)-2-((tert-Butyldiphenylsilyl)oxy)propanal (2.05 g, 6.28 mmol) in THF (10 mL) was added dropwise and left to stir at room temperature for 18 hours. The reaction mixture was diluted in ethyl acetate, washed with water, brine, dried (MgSO₄) and concentrated under reduced pressure to give the title compound as a colourless oil (3.31 g, 100%). No further purification was required.

¹H NMR (500 MHz, CDCl₃) δ 7.84-7.60 (4H, m), 7.50-7.30 (6H, m), 6.92 (1H, dd, J=15.5 and 4.5 Hz), 6.03 (1H, dd, J=15.6 and 1.7 Hz), 4.53-4.44 (1H, m), 4.33-4.11 (2H, m), 1.32 (1H, t, J=7.1 Hz), 1.15 (3H, d, J=6.5 Hz), 1.11 (9H, s).

### Ethyl (S)-4-((tert-butyldiphenylsilyl)oxy)pentanoate

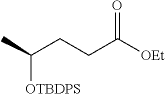

General procedure 12 was applied to ethyl (S,E)-4-((tert-butyldiphenylsilyl)oxy)pent-2-enoate (3.31 g, 8.65 mmol) and palladium on carbon (331 mg, 3.11 mmol) in ethanol (50 mL), stirred for 3 hours at room temperature under a hydrogen atmosphere. The resulting mixture was filtered, washing with ethanol (3×2.0 mL). The filtrate was concentrated under reduced pressure to give the crude product. The crude material was purified by flash column chromatography eluting with ethyl acetate (10%) in petroleum ether to yield the title compound as a colourless oil (1.94 g, 60%).

¹H NMR (500 MHz, CDCl₃) δ 7.77-7.61 (4H, m), 7.43-7.36 (6H m), 4.26-4.07 (2H, m), 3.97-3.90 (1H, m), 2.45-2.33 (2H, m), 1.86-1.75 (2H, m), 1.26 (3H, t, J=6.7 Hz), 1.08-1.05 (12H, m).

### (S)-4-((tert-Butyldiphenylsilyl)oxy)pentan-1-ol

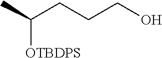

To a stirred solution of ethyl (S)-4-((tert-butyldiphenylsilyl)oxy)pentanoate (1.94 g, 5.67 mmol) in THF (16 mL) was added lithium aluminium hydride (1M, 6.80 ml, 6.80 mmol) dropwise at 0° C. and left to stir for 2 hours. The reaction mixture was extracted with ethyl acetate, then washed with water, brine, dried (MgSO₄) and concentrated under reduced pressure to give the title compound as a colourless oil (1.64 g, 84%). No further purification was required.

¹H NMR (500 MHz, CDCl₃) δ 7.75-7.65 (4H, m), 7.49-7.35 (6H, m), 4.05-3.82 (1H, m), 3.70-3.42 (2H, m), 1.68-1.47 (4H, m), 1.14-1.05 (12H, m).

### (S)-1-(2-((4-((tert-Butyldiphenylsilyl)oxy)pentyl)oxy)-4-nitrophenyl)-4-methylpiperazine

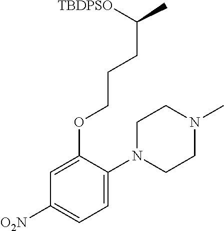

General procedure 22 was applied to 2-(4-methylpiperazin-1-yl)-5-nitrophenol (1.14 g, 4.00 mmol), (S)-4-((tert-Butyldiphenylsilyl)oxy)pentan-1-ol (1.64 g, 4.79 mmol), DIAD (940 μL, 4.79 mmol) and triphenylphosphine (1.26 g, 4.79 mmol) in THF (48 mL). The crude product was purified by flash column chromatography eluting with methanol (10%) in dichloromethane to afford the title compound as a colourless oil (1.89 g, 84%).

¹H NMR (500 MHz, CDCl₃) δ 7.86 (1H, dd, J=8.6 and 2.5 Hz), 7.73-7.66 (5H, m), 7.62-7.54 (6H, m), 7.00 (1H, d, J=8.6 Hz), 4.03-3.87 (3H, m), 3.28-3.22 (4H, m), 2.62-2.52 (4H, m), 2.33 (3H, s), 1.95-1.62 (4H, m), 1.17 (3H, d, J=6.1 Hz), 1.07 (9H, s).

### (S)-3-((4-((tert-Butyldiphenylsilyl)oxy)pentyl)oxy)-4-(4-methylpiperazin-1-yl)aniline

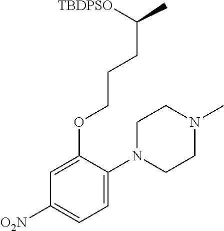

General procedure 12 was applied to (S)-1-(2-((4-((tert-butyldiphenylsilyl)oxy)pentyl)oxy)-4-nitrophenyl)-4-methylpiperazine (1.89 g, 3.36 mmol) and palladium on carbon (189 mg, 1.78 mmol) in ethanol (50 mL) was stirred for 3 hours at room temperature under an hydrogen atmosphere. The resulting mixture was filtered, washing with ethanol (3×2.0 mL). The filtrate was concentrated under reduced pressure to afford the title compound as a colourless oil (1.74 g, 97%).

¹H NMR (500 MHz, CDCl₃) δ 8.35 (1H, d, J=2.5 Hz), 7.99 (1H, dd, J=8.6 and 2.5 Hz), 7.59-7.52 (5H, m), 7.49-7.32 (6H, m), 3.81-3.67 (3H, m), 3.59-3.52 (2H, m), 3.45-3.38 (2H, m), 3.21-3.14 (2H, m), 3.04-2.96 (2H, m), 2.94-2.85 (2H, m), 2.75 (3H, s), 2.10-1.99 (2H, m), 0.99-0.94 (12H, m).

### (S)-2-((3-((4-hydroxypentyl)oxy)-4-(4-methylpiperazin-1-yl)phenyl)amino)-5-((triisopropylsilyl)ethynyl)pyrido[2,3-d]pyrimidin-7(8H)-one

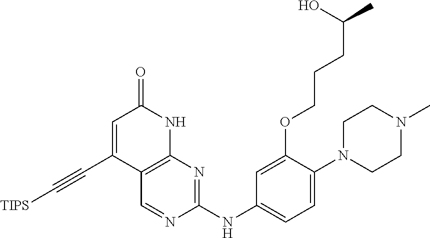

General procedure 13 was applied to (S)-3-((4-((tert-butyldiphenylsilyl)oxy)pentyl)oxy)-4-(4-methylpiperazin-1-yl)aniline (1.74 g, 3.27 mmol) with 2-(methylsulfonyl)-5-((triisopropylsilyl)ethynyl)pyrido[2,3-d]pyrimidin-7(8H)-one (1.30 g, 3.21 mmol) and trifluoroacetic acid (294 μL, 3.85 mmol) in acetonitrile (32 mL). The crude material was purified by flash column chromatography eluting with methanol (10%) in dichloromethane to yield the title compound as an orange solid (478 mg, 24%).

### (R)-8-methyl-3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one

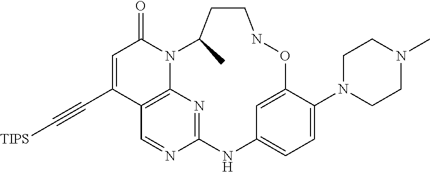

According to general procedure 20 (S)-2-((3-((4-hydroxypentyl)oxy)-4-(4-methylpiperazin-1-yl)phenyl)amino)-5-((triisopropylsilyl)ethynyl)pyrido[2,3-d]pyrimidin-7(8H)-one (478 mg, 0.773 mmol), DIAD (606 μL, 3.09 mmol) and triphenylphosphine (810 mg, 3.09 mmol) in THF (32 mL). The crude product was purified by flash column chromatography eluting with methanol (10%) in dichloromethane to afford the title compound as a yellow solid (8.5 0 mg, 18%).

¹H NMR (500 MHz, CDCl₃) δ 8.73 (1H, s), 8.22-8.14 (1H, m), 8.09-7.97 (1H, m), 6.78 (1H, d, J=8.4 Hz), 6.51 (1H, dd, J=8.4 and 2.4 Hz), 6.45 (1H, s), 4.48-4.35 (1H, m), 3.16-2.91 (4H, m), 2.65-2.50 (4H, m), 2.30 (3H, s), 2.06-2.03 (2H, m), 1.66-1.44 (4H, m), 1.20-1.00 (24H, m).

### (R)-1⁵-ethynyl-8-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-1⁷-one

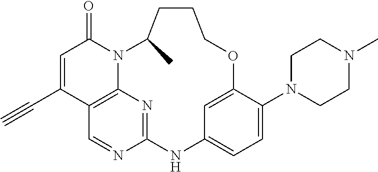 [SMILES: C#Cc1cc(=O)n2c3nc(ncc13)Nc1ccc(N3CCN(C)CC3)c(c1)OCCC[C@H]2C]

General procedure 15 was applied to (R)-8-methyl-3⁴-(4-methylpiperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one (85.0 mg, 0.142 mmol) with potassium fluoride (82.0 mg, 1.42 mmol) in DMF (5.0 mL). The crude material was purified by flash chromatography eluting with methanol (10%) in dichloromethane to yield the title compound as a yellow solid (12.0 mg, 19%).

(ES, m/z): [M+1]+=443

¹H NMR (500 MHz, CDCl₃) δ 8.83 (1H, s), 8.36-8.23 (1H, s), 7.70-7.66 (1H, m), 6.89 (1H, d, J=8.4 Hz), 6.63-6.56 (2H, m), 5.46-5.31 (1H, m), 4.57-4.46 (1H, m), 4.22-4.13 (1H, m), 3.63 (1H, s), 3.19-3.05 (4H, m), 2.69-2.59 (4H, m), 2.39 (3H, s), 2.19-2.08 (2H, m), 2.01-1.88 (2H, m), 1.69-1.54 (3H, m).

### Example 240

### [2-(2-Bromoethoxy)ethoxy](tert-butyl)diphenylsilane

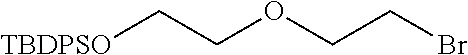

General procedure 21 was applied to 2-(2-bromoethoxy)ethanol (9.00 g, 53.2 mmol), imidazole (7.25 g, 106 mmol), and DMAP (1.30 g, 10.6 mmol) and tert-butyldiphenylsilyl chloride (17.5 g, 63.9 mmol) in DMF (90 mL). The residue was purified by reverse-phase flash column chromatography eluting with acetonitrile (50-100%) in water (10 mmol/L ammonium carbonate) to yield the title compound as a colourless oil (12.0 g, crude).

### tert-Butyldiphenyl(2-[2-(4,4,5,5-tetramethyl-1,3,2-dioxaborolan-2-yl)ethoxy]ethoxysilane

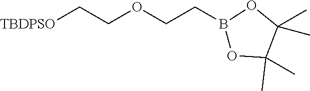

To a solution of [2-(2-bromoethoxy)ethoxy](tert-butyl)diphenylsilane (12.0 g, 29.4 mmol, 1.0 eq.), 4,4,5,5-tetramethyl-2-(4,4,5,5-tetramethyl-1,3,2-dioxaborolan-2-yl)-1,3,2-dioxaborolane (11.2 g, 44.2 mmol, 1.5 eq.) and lithium methoxide (2.24 g, 58.9 mmol, 2 eq.) in DMF (120 mL) was added triphenylphosphine (1.00 g, 3.83 mmol, 0.13 eq.) and copper iodide (560 mg, 2.94 mmol, 0.10 eq.). After stirring overnight at room temperature under a nitrogen atmosphere the mixture was extracted with ethyl acetate (2×500 mL). The combined organic layers were washed with brine (2×300 mL), dried (Na₂SO₄) and concentrated under reduced pressure. The residue was purified by reverse-phase flash column chromatography eluting with acetonitrile (50-100%) in water (10 mmol/L ammonium carbonate) to yield the title compound as a white solid (7.00 g, 52%).

### 1-[2-(2-(2-[(tert-Butyldiphenylsilyl)oxy]ethoxyethyl)-4-nitrophenyl]-4-methylpiperazine

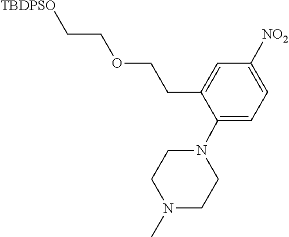

To a solution of tert-butyldiphenyl(2-[2-(4,4,5,5-tetramethyl-1,3,2-dioxaborolan-2-yl)ethoxy]ethoxysilane (7.27 g, 16.0 mmol, 1.2 eq.) and 1-(2-bromo-4-nitrophenyl)-4-methylpiperazine (4.00 g, 13.3 mmol, 1.0 eq.) in toluene (40 mL) and water (10 mL) was added sodium 2-methylpropan-2-olate (3.84 g, 40.0 mmol, 3.0 eq.) and tris(dibenzylideneacetone)dipalladium(0) (240 mg, 0.267 mmol, 0.02 eq.), RuPhos (250 mg, 0.533 mmol, 0.04 eq.). After stirring for 8 hours at 80° C. under a nitrogen atmosphere, the mixture was concentrated under reduced pressure. The residue was purified by Prep-TLC/flash column chromatography eluting with methanol (12%) in dichloromethane to afford the title compound as a yellow solid (4.00 g, 55%).

### 3-(2-(2-[(tert-Butyldiphenylsilyl)oxy]ethoxyethyl)-4-(4-methylpiperazin-1-yl)aniline

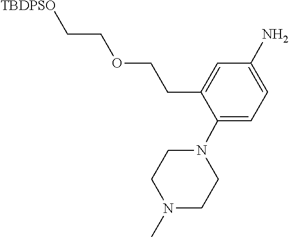

General procedure 12 was applied to 1-[2-(2-(2-[(tert-butyldiphenylsilyl)oxy]ethoxyethyl)-4-nitrophenyl]-4-methylpiperazine (2.00 g, 3.65 mmol) and palladium on carbon (400 mg, 3.76 mmol) in THF (60 mL) to yield the title compound as a brown solid (1.80 g, 95%).

### 2-([3-(2-(2-[(tert-Butyldiphenylsilyl)oxy]ethoxyethyl)-4-(4-methylpiperazin-1-yl)phenyl]amino-5-[2-(triisopropylsilyl)ethynyl]-8H-pyrido[2,3-d]pyrimidin-7-one

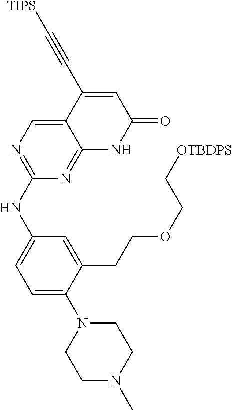

General procedure 13 was applied to 3-(2-(2-[(tert-butyldiphenylsilyl)oxy]ethoxyethyl)-4-(4-methylpiperazin-1-yl)aniline (1.80 g, 3.48 mmol) and 2-methanesulfonyl-5-[2-(triisopropylsilyl)ethynyl]-8H-pyrido[2,3-d]pyrimidin-7-one (1.13 g, 2.78 mmol) in butan-2-ol (20 mL), stirred overnight at 100° C. The residue was purified by flash column chromatography eluting with methanol (12%) in dichloromethane to afford the title compound as a brown solid (2.00 g, 68%).

### 2-((3-[2-(2-Hydroxyethoxy)ethyl]-4-(4-methylpiperazin-1-yl)phenylamino)-5-[2-(triisopropylsilyl)ethynyl]-8H-pyrido[2,3-d]pyrimidin-7-one

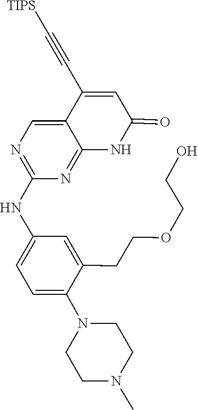

A solution of 2-([3-(2-(2-[(tert-butyldiphenylsilyl)oxy]ethoxyethyl)-4-(4-methylpiperazin-1-yl)phenyl]amino-5-[2-(triisopropylsilyl)ethynyl]-8H-pyrido[2,3-d]pyrimidin-7-one (2.00 g, 2.37 mmol, 1.0 eq.) in hydrogen chloride in 1,4-dioxane (20 mL) was stirred for 14 hours at room temperature under nitrogen atmosphere. The residue was purified by reverse-phase flash column chromatography eluting with acetonitrile (20-70%) in water (0.1% trifluoroacetic acid) to yield the title compound as a yellow solid (700 mg, 49%).

### 3⁴-(4-methylpiperazin-1-yl)-1⁵-((triisopropylsilyl)ethynyl)-1⁷,1⁸-dihydro-6-oxa-2-aza-1(2, 8)-pyrido[2,3-d]pyrimidina-3(1,3)-benzenacyclooctaphan-1⁷-one

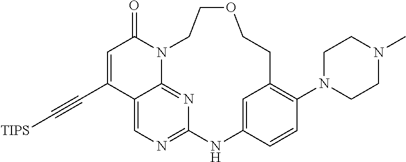

General procedure 20 was applied to 2-((3-[2-(2-hydroxyethoxy)ethyl]-4-(4-methylpiperazin-1-yl)phenylamino)-5-[2-(triisopropylsilyl)ethynyl]-8H-pyrido[2,3-d]pyrimidin-7-one (200 mg, 0.331 mmol), triphenylphosphine (347 mg, 1.32 mmol) and DEAD (230 mg, 1.32 mmol) in THF (4 mL). The residue was purified by reverse-phase flash column chromatography eluting with acetonitrile (30-80%) in water (0.1% trifluoroacetic acid) to yield the title compound as a yellow solid (150 mg, 77%).

### 1⁵-Ethynyl-3⁴-(4-methylpiperazin-1-yl)-1⁷,1⁸-dihydro-6-oxa-2-aza-1(2,8)-pyrido[2,3-d]pyrimidina-3(1,3)-benzenacyclooctaphan-1⁷-one

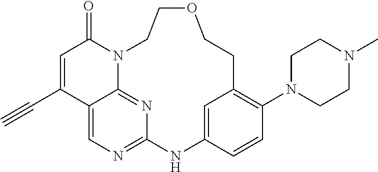 [SMILES: C#Cc1cc(=O)n2c3nc(ncc13)Nc1ccc(N3CCN(C)CC3)c(c1)CCOCC2]

General procedure 15 was applied to 3⁴-(4-methylpiperazin-1-yl)-15-((triisopropylsilyl)ethynyl)-1⁷,1⁸-dihydro-6-oxa-2-aza-1(2,8)-pyrido[2,3-d]pyrimidina-3(1,3)-benzenacyclooctaphan-1⁷-one (160 mg, 0.267 mmol) and potassium fluoride (158 mg, 2.73 mmol) in THF (2.0 mL), DMF (2.0 mL) and water (160 μL), stirred for 2 hours at 60° C. The residue was purified by reverse-phase flash column chromatography eluting with acetonitrile (10-50%) in water (0.1% formic acid) to yield the title compound as a yellow solid (31.0 mg, 25%).

(ES, m/z): [M+H]⁺=431.15

¹H-NMR (400 MHz, DMSO-d₆) δ 10.21 (s, 1H), 8.77 (s, 1H), 8.68 (d, J=2.0 Hz, 1H), 6.98 (d, J=2.1 Hz, 2H), 6.54 (s, 1H), 5.05 (s, 1H), 4.31 (t, J=5.5 Hz, 2H), 3.76 (dd, J=14.0, 8.4 Hz, 4H), 3.30 (s, 2H), 2.82 (s, 6H), 2.52 (s, 2H), 2.29 (s, 3H).

### Example 241

### 2-[4-(2-Methoxyethyl)piperazin-1-yl]-5-nitrophenol

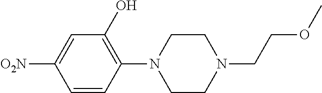 [SMILES: COCCN1CCN(c2ccc([N+](=O)[O-])cc2O)CC1]

A solution of 2-fluoro-5-nitrophenol (6.00 g, 6.36 mmol, 1.0 eq.) in 1-(2-methoxyethyl)piperazine (4.00 mL, 27.7 mmol, 4.4 eq.) was stirred for 1 hour at 130° C. The residue was purified by flash column chromatography eluting with ethyl acetate (50%) in 40-60 petroleum ether to afford the title compound as a yellow oil (6.00 g, 56%).

### 1-(2-(4-[(tert-Butyldiphenylsilyl)oxy]butoxy-4-nitrophenyl)-4-(2-methoxyethyl)piperazine

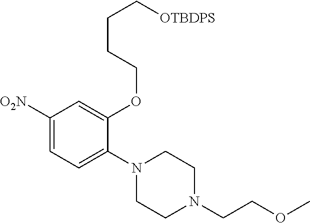

General procedure 22 was applied to 2-[4-(2-methoxyethyl)piperazin-1-yl]-5-nitrophenol (5.00 g, 17.8 mmol), 4-[(tert-butyldiphenylsilyl)oxy]butan-1-ol (6.42 g, 19.5 mmol), triphenylphosphine (9.32 g, 35.5 mmol) and DIAD (7.05 mL, 35.5 mmol) in dichloromethane (50 mL). The residue was purified by flash column chromatography eluting with ethyl acetate (10%) in 40-60 petroleum ether to afford the title compound as a yellow oil (4.00 g, 38%).

### 3-(4-[(tert-Butyldiphenylsilyl)oxy]butoxy-4-[4-(2-methoxyethyl)piperazin-1-yl]aniline

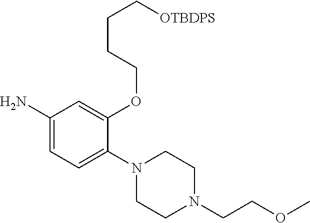

General procedure 12 was applied to 1-(2-(4-[(tert-butyldiphenylsilyl)oxy]butoxy-4-nitrophenyl)-4-(2-methoxyethyl)piperazine (4.00 g, 6.76 mmol) and palladium on carbon (10%, 800 mg) in methanol (40 mL) to yield the title compound as a light yellow oil (3.00 g, 79%).

### 2-[(3-(4-[(tert-Butyldiphenylsilyl)oxy]butoxy-4-[4-(2-methoxyethyl)piperazin-1-yl]phenyl)amino]-5-[2-(triisopropylsilyl)ethynyl]-8H-pyrido[2,3-d]pyrimidin-7-one

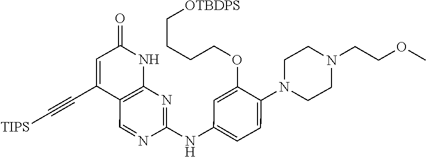

General procedure 13 was applied to 3-(4-[(tert-butyldiphenylsilyl)oxy]butoxy-4-[4-(2-methoxyethyl)piperazin-1-yl]aniline (2.50 g, 4.45 mmol), 2-methanesulfonyl-5-[2-(triisopropylsilyl)ethynyl]-8H-pyrido[2,3-d]pyrimidin-7-one (2.17 g, 5.34 mmol) and trifluoroacetic acid (1.01 g, 8.90 mmol) in 2-methyl-2-butanol (25 mL), stirred for 14 hours at 100° C. The residue was purified by reverse-phase flash column chromatography eluting with acetonitrile (50-100%) in water (0.1% trifluoroacetic acid) to afford the title compound as a light yellow solid (1.00 g, 25%).

### 2-([3-(4-Hydroxybutoxy)-4-[4-(2-methoxyethyl)piperazin-1-yl]phenyl]amino-5-[2-(triisopropylsilyl)ethynyl]-8H-pyrido[2,3-d]pyrimidin-7-one

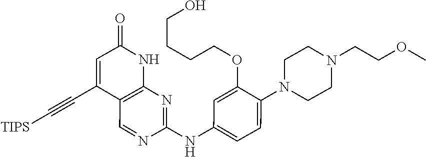

A solution of 4-[(tert-butyldiphenylsilyl)oxy]-N-[2-(4-methylpiperazin-1-yl)-5-((7-oxo-5-[2-(triisopropylsilyl)ethynyl]-8H-pyrido[2,3-d]pyrimidin-2-ylamino)phenyl]butanamide (500 mg, 0.584 mmol, 1.0 eq.) and hydrogen chloride in 1,4-dioxane (5.0 mL) was stirred for 14 hours at room temperature. The residue was purified by reverse-phase flash column chromatography eluting with acetonitrile (10-70%) in water (0.1% trifluoroacetic acid) to yield the title compound as a yellow solid (300 mg, 45%).

### 3⁴-(4-(2-Methoxyethyl)piperazin-1-yl)-1⁵-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one

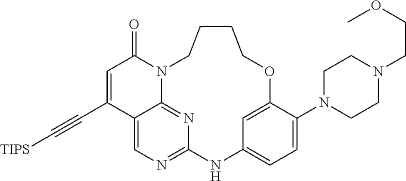

General procedure 20 was applied to 2-([3-(4-hydroxybutoxy)-4-[4-(2-methoxyethyl)piperazin-1-yl]phenyl]amino-5-[2-(triisopropylsilyl)ethynyl]-8H-pyrido[2,3-d]pyrimidin-7-one (270 mg, 0.416 mmol), triphenylphosphine (436 mg, 1.66 mmol) and DEAD (290 mg, 1.66 mmol) in THF (5.0 mL). The residue was purified by reverse-phase flash column chromatography eluting with acetonitrile (10-70%) in water (0.1% trifluoroacetic acid) to yield the title compound as a yellow solid (130 mg, 49%).

### 1⁵-Ethynyl-3⁴-(4-(2-methoxyethyl)piperazin-1-yl)-1⁷,1⁸-dihydro-4-oxa-2-aza-1(2,8)-pyrido[2,3-d]pyrimidina-3(1,3)-benzenacyclooctaphan-1⁷-one

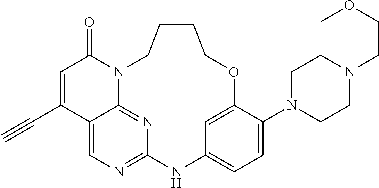 [SMILES: C#Cc1cc(=O)n2c3nc(ncc13)Nc1ccc(N3CCN(CCOC)CC3)c(c1)OCCCC2]

General procedure 15 was applied to 3⁴-(4-(2-Methoxyethyl)piperazin-1-yl)-15-((triisopropylsilyl)ethynyl)-1⁷,1⁸-dihydro-4-oxa-2-aza-1(2,8)-pyrido[2,3-d]pyrimidina-3(1,3)-benzenacyclooctaphan-1⁷-one (100 mg, 0.159 mmol) and potassium fluoride (92.1 mg, 1.59 mmol) in THF (2.0 mL), DMF (2.0 mL) and water (100 μL), stirred for 2 hours at room temperature. The residue was washed with acetonitrile/water (1/1) (3×5.0 mL) to yield the title compound as a yellow solid (33.0 mg, 43%).

(ES, m/z): [M+H]⁺=475.15

¹H-NMR (400 MHz, DMSO-d₆) 510.23 (s, 1H), 8.75 (s, 1H), 8.45 (d, J=2.3 Hz, 1H), 6.80 (d, J=8.6 Hz, 1H), 6.72 (dd, J=8.5, 2.3 Hz, 1H), 6.54 (s, 1H), 5.05 (s, 1H), 4.24 (d, J=61.6 Hz, 4H), 3.46 (t, J=5.8 Hz, 2H), 3.24 (s, 3H), 2.94 (s, 4H), 2.54 (d, J=7.4 Hz, 6H), 1.90 (d, J=31.1 Hz, 4H).

### TR-FRET Analysis

### Synthesis of Probe

### tert-butyl 4-(3-bromopropyl)piperazine-1-carboxylate

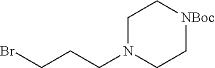 [SMILES: CC(C)(C)OC(=O)N1CCN(CCCBr)CC1]

To a stirred solution of 1-Boc-piperzine (1.00 g, 5.37 mmol) in dichloromethane (15 mL) at 0° C. was added triethylamine (596 mg, 5.90 mmol) and 1,3-dibromopropane (0.602 mL, 5.90 mmol). The reaction mixture was warmed to room temperature over 18 h. The reaction mixture was diluted with dichloromethane (30 mL) and washed with saturated aqueous sodium hydrogen carbonate solution (20 mL), water (10 mL) and brine (10 mL), dried (Na₂SO₄) and concentrated under reduced pressure. The crude material was purified by flash column chromatography eluting with ethyl acetate (30%) in 40-60 petroleum ether to yield the title compound as an oil (70%).

¹H NMR (500 MHz, CDCl₃) b 3.49 (t, J=6.6 Hz, 2H), 3.44 (t, J=5.2 Hz, 4H), 2.50 (t, J=6.9 Hz, 2H), 2.40 (t, J=5.1 Hz, 4H), 2.06-2.01 (m, 2H), 1.48 (s, 9H)

### tert-butyl 4-(3-((4-((3-chloro-4-fluorophenyl)amino)-7-methoxyquinazolin-6-yl)oxy)propyl)piperazine-1-carboxylate

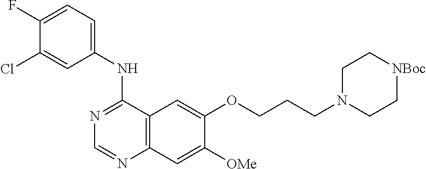 [SMILES: COc1cc2ncnc(Nc3ccc(F)c(Cl)c3)c2cc1OCCCN1CCN(C(=O)OC(C)(C)C)CC1]

To a stirred solution of 4-((3-chloro-4-fluorophenyl)amino)-7-methoxyquinazolin-6-ol (300 mg, 0.941 mmol) in DMF (3.0 mL) was added potassium carbonate (259 mg, 1.88 mmol) followed by tert-butyl 4-(3-bromopropyl)piperazine-1-carboxylate (346 mg, 1.12 mmol). The reaction mixture was heated at 80° C. for 5 h then concentrated Under reduced pressure. The residue was dissolved in dichloromethane (50 mL) and washed with brine (10 mL), dried (Na₂SO₄) and concentrated under reduced pressure.

The crude material was purified by flash column chromatography to yield the title compound (49%).

(ES, m/z): [M+H]⁺=547

¹H NMR (500 MHz, CDCl₃) δ 8.65 (s, 1H), 7.82 (dd, J=2.7, 6.5 Hz, 1H), 7.68 (s, 1H), 7.53 (ddd, J=2.7, 4.1, 8.9 Hz, 1H), 7.22 (s, 1H), 7.19 (s, 1H), 7.13 (t, J=8.7 Hz, 1H), 4.13 (t, J=6.6 Hz, 2H), 3.96 (s, 3H), 3.46-3.37 (m, 4H), 2.53 (t, J=7.0 Hz), 2.38 (t, J=5.1 Hz, 4H), 2.06 (dd, J=6.3, 13.1 Hz, 2H), 1.44 (s, 9H).

### N-(3-chloro-4-fluorophenyl)-7-methoxy-6-(3-(piperazin-1-yl)propoxy)quinazolin-4-amine

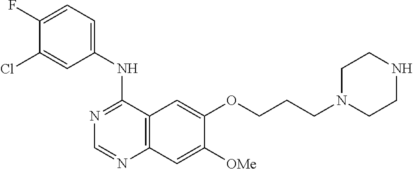 [SMILES: COc1cc2ncnc(Nc3ccc(F)c(Cl)c3)c2cc1OCCCN1CCNCC1]

Tert-butyl4-(3-((4-((3-chloro-4-fluorophenyl)amino)-7-methoxyquinazolin-6-yl)oxy)propyl)piperazine-1-carboxylate (234 mg, 0.422 mmol) in trifluoroacetic acid (1 mL) and dichloromethane (1 mL) was stirred at room temperature for 3 h. The reaction mixture was diluted with dichloromethane (20 mL), basified to pH 10, and extracted with dichloromethane (3×20 mL). The combined organic layers were dried (Na₂SO₄) and concentrated under reduced pressure to yield the title compound as yellow solid (180 mg, 91%).

¹H NMR (500 MHz, CDCl₃) δ 8.66 (s, 1H), 7.91 (dd, J=6.5, 2.6 Hz, 1H), 7.55 (ddd, J=8.4, 3.9, 2.5 Hz, 1H), 7.45 (s, 1H), 7.25 (s, 1H), 7.19-7.13 (m, 2H), 4.19 (t, J=6.6 Hz, 2H), 4.00 (d, J=1.3 Hz, 3H), 2.93 (t, J=4.8 Hz, 4H), 2.59 (t, J=7.0 Hz, 2H), 2.55-2.44 (m, 4H), 2.12 (q, J=7.0 Hz, 2H).

### Methyl 4-azidobutanoate

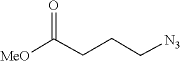

To a stirred solution of methyl 4-chlorobutyrate (500 mg, 3.66 mmol) in DMSO (3.0 mL) was added sodium azide (333 mg, 5.10 mmol). The reaction mixture was heated at 45° C. for 24 h then cooled down to room temperature and quenched with water (10 mL). The aqueous layer was extracted with diethyl ether (3×30 mL). The combined organic layers were dried (Na₂SO₄) and concentrated under reduced pressure to yield the title compound as a colourless oil (95%).

¹H NMR (500 MHz, CDCl₃) b 3.70 (d, J=3.0 Hz, 3H), 3.37 (td, J=6.7, 2.7 Hz, 2H), 2.44 (td, J=7.3, 2.7 Hz, 2H), 1.93 (pd, J=7.1, 2.9 Hz, 2H).

### 4-azidobutanoic acid

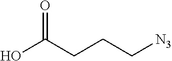

Methyl 4-azidobutanoate (500 mg, 3.49 mmol) was dissolved in 2M aqueous sodium hydroxide solution (6.99 mL), a few drops of methanol were added to the reaction and it was stirred at room temperature overnight. The reaction mixture was concentrated under reduced pressure to remove the methanol. The aqueous layer was washed with diethyl ether (3×20 mL), acidified with 6M HCl to pH 1 and then extracted with diethyl ether (3×35 mL). The combined organic layers were dried (MgSO₄) and concentrated under reduced pressure to yield the title compound as colourless oil (98%).

¹H NMR (500 MHz, CDCl₃) b 3.40 (t, J=6.7 Hz, 2H), 2.49 (t, J=7.2 Hz, 2H), 1.93 (p, J=6.9 Hz, 2H). ¹³C NMR (126 MHz, CDCl₃) δ 178.81, 50.47, 30.91, 23.95

### 4-azido-1-(4-(3-((4-((3-chloro-4-fluorophenyl)amino)-7-methoxyquinazolin-6-yl)oxyl)propyl)piperazin-1-yl)butan-1-one

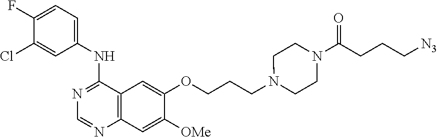 [SMILES: COc1cc2ncnc(Nc3ccc(F)c(Cl)c3)c2cc1OCCCN1CCN(C(=O)CCCN=[N+]=[N-])CC1]

To a stirred solution of 4-azidobutanoic acid (16.0 mg, 0.123 mmol) in dichloromethane (0.6 mL) was added N,N-diisopropyl-N-ethylamine (43.4 mg, 0.336 mmol) followed by HATU (63.0 mg, 0.168 mmol). The reaction mixture stirred at rt for 10 minutes, N-(3-chloro-4-fluorophenyl)-7-methoxy-6-(3-(piperazin-1-yl)propoxy)quinazolin-4-amine (50.0 mg, 0.110 mmol) was added and the reaction mixture stirred at room temperature for 18 h. The reaction was diluted with dichloromethane (25 mL), washed with water (7 mL), dried (MgSO₄) and concentrated under reduced pressure. The crude material was purified by flash column chromatography using SiO₂ NH₂, eluting with ethyl acetate to yield the title compound as white solid (40.0 mg, 65%).

¹H NMR (500 MHz, CDCl₃) δ 8.65 (s, 1H), 7.82 (dd, J=6.5, 2.7 Hz, 1H), 7.61 (s, 1H), 7.53 (ddd, J=8.9, 4.1, 2.7 Hz, 1H), 7.23 (s, 1H), 7.17 (s, 1H), 7.14 (t, J=8.8 Hz, 1H), 4.14 (t, J=6.5 Hz, 2H), 3.97 (s, 3H), 3.61 (t, J=5.0 Hz, 2H), 3.47 (dt, J=6.5, 3.9 Hz, 2H), 3.36 (t, J=6.4 Hz, 2H), 2.56 (t, J=7.0 Hz, 2H), 2.48-2.40 (m, 4H), 2.38 (d, J=7.2 Hz, 2H), 2.08 (q, J=6.8 Hz, 2H), 1.90 (p, J=6.8 Hz, 2H).

### 4-amino-1-(4-(3-((4-((3-chloro-4-fluorophenyl)amino)-7-methoxyquinazolin-6-yl)oxy)propyl)piperazin-1-yl)butan-1-one

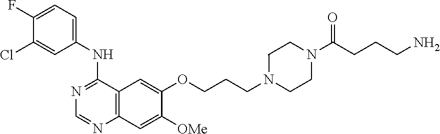 [SMILES: COc1cc2ncnc(Nc3ccc(F)c(Cl)c3)c2cc1OCCCN1CCN(C(=O)CCCN)CC1]

To a stirred solution of 4-azido-1-(4-(3-((4-((3-chloro-4-fluorophenyl)amino)-7-methoxyquinazolin-6-yl)oxy)propyl)piperazin-1-yl)butan-1-one (49.0 mg, 88.0 μmol) in methanol (2 mL) was added tin (II) chloride (25.0 mg, 0.132 mmol). The reaction was stirred at room temperature overnight, then concentrated under reduced pressure. The residue was suspended in ethyl acetate (15 mL) and washed with 5M aqueous potassium fluoride solution (10 mL). The aqueous layer was further extracted with ethyl acetate (10 mL). The combined organic layers were washed with brine, dried (Na₂SO₄) and concentrated under reduced pressure. The crude material was purified by flash column chromatography using amino silica eluting with methanol (0-20%) in dichloromethane to yield the titled compound as an oil (35 mg, 75%).

### Methyl (E)-3-(1H-pyrrol-2-yl)acrylate

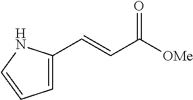 [SMILES: COC(=O)/C=C/c1ccc[nH]1]

To a stirred solution of pyrrole-2-carboxaldehyde (1.03 g, 10.4 mmol) in dichloromethane (50 mL) was added methyl (triphenylphosphoranylidene)acetate (7.07 g, 20.7 mmol) and the reaction stirred at room temperature for 24 h. The reaction mixture was concentrated under reduced pressure. The crude material was purified by flash column chromatography eluting with ethyl acetate (20-30%) in 40-60 petroleum ether to yield the title compound as an off white solid (60%).

¹H NMR (500 MHz, CDCl₃) δ 8.96 (s, 1H), 7.51 (d, J=15.9 Hz, 1H), 6.86 (td, J=1.4, 2.8 Hz, 1H), 6.49 (ddd, J=1.4, 2.5, 3.8 Hz, 1H), 6.21 (dt, J=2.5, 3.7 Hz, 1H), 5.98 (d, J=15.9 Hz, 1H), 3.71 (s, 3H).

### Methyl 3-(1H-pyrrol-2-yl)propanoate

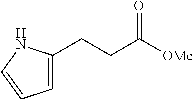 [SMILES: COC(=O)CCc1ccc[nH]1]

To a stirred solution of methyl (E)-3-(1H-pyrrol-2-yl)acrylate (800 mg, 5.29 mmol) in methanol (25 mL) was added 10% palladium on carbon (56.0 mg). The reaction was stirred under hydrogen for 3 h then filtered through Celite and concentrated under reduced pressure. The crude material was purified by flash column chromatography eluting with ethyl acetate (20%) in 40-60 petroleum ether to yield the title compound as pale yellow oil (650 mg, 81%).

(ES, m/z): [M+H]⁺=154

¹H NMR (500 MHz, CDCl₃) δ 8.46 (s, 1H), 6.60 (td, J=1.5, 2.7 Hz, 1H), 6.03 (q, J=2.8 Hz, 1H), 5.84 (ddd, J=1.6, 2.6, 3.3 Hz, 1H), 3.62 (s, 3H), 2.84 (t, J=6.8 Hz, 2H), 2.57 (t, J=6.8 Hz, 2H).

### 4,4-Difluoro-1,3-dimethyl-4-bora-3a,4a-diaza-s-indacene-2-propionic acid methyl ester

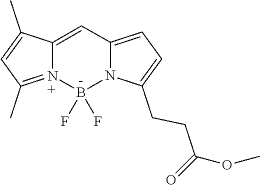

To a stirred solution of methyl 3-(1H-pyrrol-2-yl)propanoate (500 mg, 3.26 mmol), 3,5-dimethylpyrrole-2-carboxaldehyde (442 mg 3.59 mmol) in dichloromethane (25 mL) at 0° C. was added phosphorus (v) oxychloride (335 μL, 3.59 mmol) dropwise. After stirring for 30 minutes at 0° C. the reaction was stirred for 7 h at room temperature. The resultant dark solution was cooled down to 0° C. and BF₃·OEt₂ (1.59 mL, 13.0 mmol) and N,N-diisopropyl-N-ethylamine (2.15 mL, 13.0 mmol) were added and the mixture stirred overnight at room temperature. The reaction was quenched with water (50 mL) then filtered through Celite, washing with dichloromethane (50 mL). The filtrate was diluted with dichloromethane (50 mL) and separated. The aqueous layer was extracted with dichloromethane (3×30 mL). The combined organic layers were dried (Na₂SO₄) and concentrated under reduced pressure. The crude material was purified by flash column chromatography eluting with dichloromethane to yield the title compound as dark red solid (680 mg, 75%).

(ES, m/z): [M+H]⁺=307

¹H NMR (500 MHz, CDCl₃): δ=7.09 (s, 1H), 6.89 (d, J=3.9 Hz, 1H), 6.27 (d, J=4.0 Hz, 1H), 6.12 (s, 1H), 3.70 (s, 3H), 3.31 (t, J=7.6 Hz, 2H), 2.78 (t, J=7.5 Hz, 2H), 2.58 (s, 3H), 2.26 ppm (s, 3H).

### 4,4-difluoro-1,3-dimethyl-4-bora-3a,4a-diaza-s-indacene-2-propionic acid

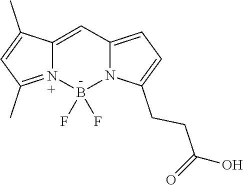

A solution of 4,4-Difluoro-1,3-dimethyl-4-bora-3a,4a-diaza-s-indacene-2-propionic acid methyl ester (650 mg) and concentrated hydrochloric acid in THF and water was stirred at room temperature for 24 h. Dichloromethane (150 mL) was added and the phases separated. The aqueous layer was extracted with dichloromethane (2×150 mL). The combined organic layers were washed with brine, dried (MgSO₄) and concentrated under reduced pressure. The crude material was purified by flash column chromatography eluting with dichloromethane (1% acetic acid) to yield the title compound as red solid (486 mg, 78%).

(ES, m/z): [M+H]⁺=293

¹H NMR (500 MHz, DMSO-d⁶): δ=12.30 (s, 1H), 7.70 (s, 1H), 7.09 (d, J=4.0 Hz, 1H), 6.38 (d, J=4.0 Hz, 1H), 6.31 (s, 1H), 3.10-3.05 (t, J=7.1 Hz, 2H), 2.64 (t, J=8.5 Hz, 2H), 2.47 (s, 3H), 2.26 ppm (s, 3H); ¹³C NMR (126 MHz, DMSO-d⁶): δ=173.4, 159.5, 156.9, 144.3, 134.5, 133.0, 128.8, 125.4, 120.4, 116.5, 32.3, 23.5, 14.5, 11.0

### N-(4-(4-(3-((4-((3-chloro-4-fluorophenyl)amino)-7-methoxyquinazolin-6-yl)oxy)propyl)piperazin-1-yl)-4-oxobutyl)-3-(5,5-difluoro-7,9-dimethyl-5H-5I4,6I4-dipyrrolo[1,2-c:2′,1′-f][1,3,2]diazaborinin-3-yl)propanamide

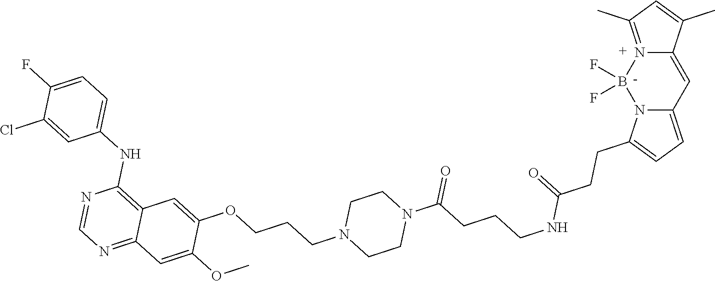

To a stirred solution of 4,4-difluoro-1,3-dimethyl-4-bora-3a,4a-diaza-s-indacene-2-propionic acid (38.0 mg, 0.136 mmol) in DMF (2 mL) was added N,N-diisopropyl-N-ethylamine (50.1 mg, 0.389 mmol) and HATU (74.0 mg, 0.194 mmol). The reaction was stirred at room temperature for 30 minutes then 4-amino-1-(4-(3-((4-((3-chloro-4-fluorophenyl)amino)-7-methoxyquinazolin-6-yl)oxy)propyl)piperazin-1-yl)butan-1-one (69.0 mg, 0.129 mmol) was added and the reaction mixture stirred overnight. The reaction mixture was concentrated under reduced pressure. The residue was dissolved in dichloromethane (30 mL), washed with saturated aqueous sodium hydrogen carbonate solution (2×10 mL). The aqueous layer was back extracted, the combined organic layers were dried (Na₂SO₄) and concentrated under reduced pressure. The crude material was purified by flash column chromatography using by amino silica, eluting with methanol in ethyl acetate to yield the title compound as red solid (62%).

¹H NMR (500 MHz, CDCl₃) δ 8.63 (s, 1H), 8.00 (s, 1H), 7.89 (dd, J=6.6, 2.7 Hz, 1H), 7.57 (dt, J=9.1, 3.5 Hz, 1H), 7.37 (s, 1H), 7.22 (s, 1H), 7.10 (t, J=8.8 Hz, 1H), 7.03 (s, 1H), 6.82 (d, J=4.0 Hz, 1H), 6.22 (d, J=4.0 Hz, 1H), 6.14 (t, J=5.9 Hz, 1H), 6.03 (s, 1H), 4.19 (t, J=6.6 Hz, 2H), 3.98 (s, 3H), 3.70-3.65 (m, 2H), 3.48 (s, 2H), 3.25 (p, J=7.9, 7.1 Hz, 4H), 2.66 (t, J=6.8 Hz, 2H), 2.62-2.57 (m, 2H), 2.56-2.50 (m, 3H), 2.49 (s, 3H), 2.30 (t, J=6.8 Hz, 2H), 2.20 (s, 3H), 2.11 (t, J=6.7 Hz, 2H), 1.80 (p, J=6.7 Hz, 2H); ¹⁹F NMR (471 MHz, CDCl₃) δ −121.39, −144.25 (dd, J=66.7, 33.0 Hz); ¹¹B NMR (160 MHz, CDCl₃) δ 0.91 (t, J=34.4 Hz).

### TR-FRET Procedure

Compounds (dissolved to 100 mM in DMSO) were dispensed into black low volume 384 well assay plates (Corning) over a final concentration range of 100000, 30000, 10000, 3000, 1000, 300, 100, 30, 10, and 3 nM using an Echo 550 (Labcyte). Positive control compound and DMSO as a negative control were dispensed into the first and last well, respectively. Each well was backfilled with DMSO to a final volume of 200 nL, resulting in final assay DMSO concentrations of 1%. 19.8 μl of premixed solution containing final assay concentration of CtermHisTag-mEGFR (Wild type, Triple Mutant—R797S, L858R, T790M) (1.25 nM), Probe (N-(4-(4-(3-((4-((3-chloro-4-fluorophenyl)amino)-7-methoxyquinazolin-6-yl)oxy)propyl)piperazin-1-yl)-4-oxobutyl)-3-(5,5-difluoro-7,9-dimethyl-5H-5I4,6I4-dipyrrolo[1,2-c:2′,1′-f][1,3,2]diazaborinin-3-yl)propenamide) (100 nM), and Tb-anti-His Antibody 61HI2TLF (Cisbio Assay) (1:100 dilution) in a buffer containing 20 mM Tris pH 7.5, 100 mM NaCl, 100 μg/ml bovine serum albumin, was added to each well and incubated with shaking at room temperature for 30 mins. Plates were read using a PheraStar FS (BMG Labtech) at Ex337 nm Ex490/520 nm. The data were analysed using Graphpad Prism/Dotmatics Studies Software. Assays conducted in technical replicate and repeated as a biological duplicate.

TR-FRET Results

| Example Number | EGFR Triple Mutant (nM) | EGFR Wild-Type (μM) |
|---|---|---|
| 1 | 100-1,000 | 1-10 |
| 2 | <20 | >10 |
| 3 | 100-1,000 | >10 |
| 4 | <20 | <1 |
| 5 | 100-1,000 | 1-10 |
| 6 | <20 | <1 |
| 7 | <20 | 1-10 |
| 9 | <20 | <1 |
| 10 | 100-1,000 | <1 |
| 11 | 1,000-50,000 | 1-10 |
| 12 | <20 | <1 |
| 13 | <20 | <1 |
| 14 | <20 | <1 |
| 15 | <20 | <1 |
| 16 | <20 | <1 |
| 17 | <20 | <1 |
| 18 | <20 | <1 |
| 19 | 20-100 | <1 |
| 20 | <20 | <1 |
| 21 | <20 | <1 |
| 22 | <20 | <1 |
| 23 | <20 | <1 |
| 24 | <20 | <1 |
| 25 | <20 | <1 |
| 26 | <20 | <1 |
| 27 | <20 | <1 |
| 28 | <20 | <1 |
| 29 | <20 | <1 |
| 30 | <20 | <1 |
| 31 | <20 | <1 |
| 32 | <20 | <1 |
| 33 | <20 | <1 |
| 34 | <20 | <1 |
| 35 | <20 | <1 |
| 36 | 20-100 | 1-10 |
| 37 | <20 | <1 |
| 38 | 20-100 | <1 |
| 39 | <20 | <1 |
| 40 | <20 | <1 |
| 41 | 100-1,000 | >10 |
| 42 | 100-1,000 | 1-10 |
| 43 | 20-100 | 1-10 |
| 44 | 20-100 | <1 |
| 45 | 20-100 | <1 |
| 46 | 20-100 | 1-10 |
| 47 | 20-100 | <1 |
| 48 | 100-1,000 | >10 |
| 49 | 20-100 | <1 |
| 50 | 20-100 | <1 |
| 51 | <20 | <1 |
| 52 | 20-100 | <1 |
| 53 | 20-100 | 1-10 |
| 54 | 100-1,000 | >10 |
| 55 | 20-100 | 1-10 |
| 56 | 20-100 | >10 |
| 57 | 20-100 | >10 |
| 58 | 100-1,000 | >10 |
| 59 | 20-100 | 1-10 |
| 60 | 100-1,000 | 1-10 |
| 61 | 20-100 | <1 |
| 62 | 20-100 | 1-10 |
| 63 | 20-100 | <1 |
| 64 | 20-100 | 1-10 |
| 65 | 20-100 | 1-10 |
| 66 | 20-100 | 1-10 |
| 67 | <20 | <1 |
| 68 | <20 | <1 |
| 69 | <20 | <1 |
| 70 | <20 | <1 |
| 71 | 100-1,000 | >10 |
| 72 | 20-100 | <1 |
| 73 | 20-100 | 1-10 |
| 74 | 100-1,000 | >10 |
| 75 | <20 | >10 |
| 76 | 20-100 | <1 |
| 77 | 20-100 | >10 |
| 78 | <20 | <1 |
| 79 | <20 | <1 |
| 80 | <20 | <1 |
| 81 | <20 | <1 |
| 82 | <20 | <1 |
| 83 | 100-1,000 | >10 |
| 84 | 20-100 | >10 |
| 85 | 20-100 | 1-10 |
| 86 | <20 | <1 |
| 87 | <20 | <1 |
| 88 | 100-1,000 | 1-10 |
| 89 | <20 | <1 |
| 90 | 20-100 | 1-10 |
| 91 | <20 | <1 |
| 92 | <20 | 1-10 |
| 93 | <20 | 1-10 |
| 94 | <20 | 1-10 |
| 95 | <20 | 1-10 |
| 96 | <20 | <1 |
| 97 | <20 | >10 |
| 98 | <20 | 1-10 |
| 99 | <20 | <1 |
| 100 | 20-100 | 1-10 |
| 101 | 1,000-50,000 | >10 |
| 102 | 100-1,000 | 1-10 |
| 103 | 20-100 | 1-10 |
| 104 | 100-1,000 | >10 |
| 105 | 100-1,000 | >10 |
| 106 | 100-1,000 | >10 |
| 107 | 100-1,000 | >10 |
| 108 | 100-1,000 | >10 |
| 109 | 20-100 | 1-10 |
| 110 | 20-100 | <1 |
| 111 | 100-1,000 | 1-10 |
| 112 | 20-100 | 1-10 |
| 113 | 20-100 | 1-10 |
| 114 | 20-100 | <1 |
| 115 | 20-100 | <1 |
| 116 | 100-1,000 | 1-10 |
| 117 | 100-1,000 | >10 |
| 118 | <20 | <1 |
| 119 | 20-100 | >10 |
| 120 | 100-1,000 | 1-10 |
| 121 | 100-1,000 | 1-10 |
| 122 | 20-100 | <1 |
| 123 | 100-1,000 | >10 |
| 124 | 20-100 | >10 |
| 125 | 100-1,000 | |
| 126 | <20 | <1 |
| 127 | <20 | <1 |
| 128 | <20 | <1 |
| 129 | <20 | <1 |
| 130 | 100-1,000 | >10 |
| 131 | 20-100 | 1-10 |
| 132 | <20 | <1 |
| 133 | <20 | <1 |
| 134 | 100-1,000 | >10 |
| 135 | <20 | <1 |
| 136 | <20 | <1 |
| 137 | <20 | <1 |
| 138 | 1,000-50,000 | 1-10 |
| 139 | <20 | <1 |
| 140 | 1,000-50,000 | 1-10 |
| 141 | 20-100 | >10 |
| 142 | 100-1,000 | >10 |
| 143 | 20-100 | >10 |
| 144 | 20-100 | <1 |
| 145 | <20 | >10 |
| 146 | 1,000-50,000 | >10 |
| 147 | 100-1,000 | >10 |
| 148 | 100-1,000 | >10 |
| 149 | 20-100 | 1-10 |
| 150 | <20 | 1-10 |
| 151 | 20-100 | 1-10 |
| 152 | 1,000-50,000 | >10 |
| 153 | 1,000-50,000 | >10 |
| 154 | 1,000-50,000 | >10 |
| 155 | 100-1,000 | 1-10 |
| 156 | 1,000-50,000 | >10 |
| 157 | 1,000-50,000 | >10 |
| 158 | 1,000-50,000 | >10 |
| 159 | 20-100 | 1-10 |
| 160 | 20-100 | 1-10 |
| 161 | 1,000-50,000 | >10 |
| 162 | 1,000-50,000 | >10 |
| 163 | 1,000-50,000 | >10 |
| 164 | 100-1,000 | >10 |
| 165 | 100-1,000 | >10 |
| 166 | 1,000-50,000 | >10 |
| 167 | 100-1,000 | >10 |
| 168 | 1,000-50,000 | >10 |
| 169 | 100-1,000 | 1-10 |
| 170 | 100-1,000 | >10 |
| 171 | 100-1,000 | >10 |
| 172 | 100-1,000 | >10 |
| 173 | 1,000-50,000 | >10 |
| 174 | 1,000-50,000 | >10 |
| 175 | 100-1,000 | >10 |
| 176 | 100-1,000 | <1 |
| 177 | 100-1,000 | 1-10 |
| 178 | 20-100 | <1 |
| 179 | 20-100 | 1-10 |
| 180 | 100-1,000 | 1-10 |
| 18 | 100-1,000 | >10 |
| 182 | 100-1,000 | >10 |
| 183 | 100-1,000 | >10 |
| 184 | 1,000-50,000 | 1-10 |
| 185 | 1,000-50,000 | >10 |
| 186 | 1,000-50,000 | >10 |
| 187 | 20-100 | 1-10 |
| 188 | <20 | <1 |
| 189 | <20 | <1 |
| 190 | 100-1,000 | >10 |
| 191 | 20-100 | 1-10 |
| 192 | 100-1,000 | >10 |
| 193 | 20-100 | >10 |
| 194 | 20-100 | <1 |
| 195 | 20-100 | <1 |
| 196 | 1,000-50,000 | >10 |
| 197 | 100-1,000 | >10 |
| 198 | 100-1,000 | >10 |
| 199 | 1,000-50,000 | >10 |
| 200 | <20 | <1 |
| 201 | <20 | <1 |
| 202 | 1,000-50,000 | >10 |
| 203 | 100-1,000 | <1 |
| 204 | 100-1,000 | <1 |
| 205 | 100-1,000 | <1 |
| 206 | 100-1,000 | <1 |
| 207 | <20 | <1 |
| 208 | <20 | <1 |
| 209 | 20-100 | <1 |
| 210 | 20-100 | <1 |
| 211 | 20-100 | <1 |
| 212 | 20-100 | <1 |
| 213 | 20-100 | <1 |
| 214 | 20-100 | <1 |
| 215 | <20 | <1 |
| 216 | 20-100 | <1 |
| 217 | 20-100 | <1 |
| 218 | 100-1,000 | <1 |
| 219 | 20-100 | <1 |
| 220 | <20 | <1 |
| 221 | 20-100 | 1-10 |
| 222 | <20 | <1 |
| 223 | <20 | <1 |
| 224 | 1,000-50,000 | >10 |
| 225 | 20-100 | >10 |
| 226 | 100-1,000 | <1 |
| 227 | 100-1,000 | 1-10 |
| 228 | 20-100 | 1-10 |
| 229 | 20-100 | <1 |
| 230 | <20 | <1 |
| 231 | 20-100 | <1 |
| 232 | <20 | <1 |
| 233 | <20 | <1 |
| 234 | 20-100 | <1 |
| 235 | <20 | <1 |
| 236 | <20 | <1 |
| 237 | 20-100 | <1 |
| 238 | <20 | <1 |
| 239 | <20 | <1 |
| 240 | <20 | <1 |
| 241 | <20 | <1 |

### HTRF Analysis

### HTRF Method (H1975):

H1975 cells (ATCC CRL-5908) were plated at 20,000 cells per well in 96 well plates and placed at 37° C. 5% CO₂. Once adhered (after 24 h) cells were treated with compounds dissolved in DMSO at a final concentration of 0.1% DMSO in media. Compounds were diluted in media then added to cells for 3 hours in duplicate. Compound and media were removed from the cells, then 50 ul of HTRF lysis buffer is added (lysis buffer was diluted from 4× stock to 1× in DI water, with 1% blocking agent also added). Cells were lysed on a plate shaker (2000 rpm) for 30 minutes at room temperature. pEGFR expression was monitored using the Cisbio Phospho-EGFR (Tyri068) cellular kit (64EG1PEH). Total EGFR expression was monitored using the Cisbio Total EGFR cellular kit (64NG1PEH) as per manufacturer's instructions. Fluorescence emission was read at two different wavelengths (665 nm and 620 nm) on a PheraStar. Results were calculated as ratio of pEGFR/total EGFR and then percentage of 0 μM control.

### HTRF Method (H1975 C797S):

H1975 C797S cells (ATCC CRL-5908) were genetically engineered by CRISPR editing of EGFR C797S in house and validated by NGS. Cells were plated at 20,000 cells per well in 96 well plates and placed at 37° C. 5% CO₂. Once adhered (after 24 h) cells were treated with compounds dissolved in DMSO at a final concentration of 0.1% DMSO in media. Compounds were diluted in media then added to cells for 3 hours in duplicate. Compound and media were removed from the cells, then 50 ul of HTRF lysis buffer was added (lysis buffer was diluted from 4× stock to 1× in DI water, with 1% blocking agent also added). Cells were lysed on a plate shaker (2000 rpm) for 30 minutes at room temperature. pEGFR expression was monitored using the Cisbio Phospho-EGFR (Tyr1068) cellular kit (64EG1PEH). Total EGFR expression was monitored using the Cisbio Total EGFR cellular kit (64NG1PEH) as per manufacturer's instructions. Fluorescence emission was read at two different wavelengths (665 nm and 620 nm) on a PheraStar. Results were calculated as ratio of pEGFR/total EGFR and then percentage of 0 μM control.

### HTRF Method (A431):

Cells were plated at 20,000 cells per well in 96 well plates and placed at 37° C. 5% CO₂. Once adhered (after 24 h) cells were treated with compounds dissolved in DMSO at a final concentration of 0.1% DMSO in media. Compounds were diluted in media then added to cells for 2.5 hours in duplicate, 100 ng/ml EGF (ThermoFisher, PHG0311) was added to all compound treated cells as well as a control for 30 mins. Compound and media were removed from the cells, then 50 ul of HTRF lysis buffer was added (lysis buffer was diluted from 4× stock to 1× in DI water, with 1% blocking agent also added). Cells were lysed on a plate shaker (2000 rpm) for 30 minutes at room temperature. pEGFR expression was monitored using the Cisbio Phospho-EGFR (Tyr1068) cellular kit (64EG1PEH). Total EGFR expression was monitored using the Cisbio Total EGFR cellular kit (64NG1PEH) as per manufacturer's instructions. Fluorescence emission was read at two different wavelengths (665 nm and 620 nm) on a PheraStar. Results were calculated as ratio of pEGFR/total EGFR and then percentage of 0 μM control.

HTRF Data

| Example Number | H1975 (nM) | H1975-C797S (nM) | A431 (nM) |
|---|---|---|---|
| 3 | >3,000 | >3,000 | >3,000 |
| 6 | <500 | <500 | 1,000-3,000 |
| 7 | 1,000-3,000 | 1,000-3,000 | >3,000 |
| 9 | <500 | <500 | >3,000 |
| 10 | >3,000 | >3,000 | >3,000 |
| 11 | >3,000 | >3,000 | >3,000 |
| 12 | <500 | <500 | >3,000 |
| 14 | <500 | <500 | >3,000 |
| 15 | <500 | <500 | >3,000 |
| 16 | <500 | <500 | 1,000-3,000 |
| 17 | <500 | <500 | 1,000-3,000 |
| 18 | <500 | <500 | 1,000-3,000 |
| 19 | <500 | 500-1,000 | >3,000 |
| 20 | <500 | <500 | 1,000-3,000 |
| 21 | <500 | <500 | 1,000-3,000 |
| 22 | <500 | <500 | >3,000 |
| 23 | <500 | <500 | 1,000-3,000 |
| 24 | <500 | <500 | >3,000 |
| 25 | 1,000-3,000 | 1,000-3,000 | >3,000 |
| 26 | <500 | <500 | 1,000-3,000 |
| 27 | <500 | <500 | 1,000-3,000 |
| 28 | <500 | <500 | 1,000-3,000 |
| 29 | <500 | <500 | >3,000 |
| 30 | <500 | <500 | >3,000 |
| 31 | <500 | 500-1,000 | >3,000 |
| 32 | <500 | <500 | >3,000 |
| 33 | <500 | <500 | 1,000-3,000 |
| 34 | <500 | >3,000 | >3,000 |
| 36 | 1,000-3,000 | 1,000-3,000 | >3,000 |
| 37 | <500 | 1,000-3,000 | 1,000-3,000 |
| 38 | <500 | <500 | >3,000 |
| 39 | <500 | <500 | 1,000-3,000 |
| 40 | <500 | <500 | 1,000-3,000 |
| 43 | <500 | <500 | >3,000 |
| 44 | 500-1,000 | 1,000-3,000 | >3,000 |
| 45 | <500 | <500 | >3,000 |
| 46 | <500 | <500 | >3,000 |
| 47 | <500 | 500-1,000 | >3,000 |
| 49 | <500 | 500-1,000 | >3,000 |
| 50 | <500 | 500-1,000 | >3,000 |
| 51 | <500 | <500 | 1,000-3,000 |
| 52 | <500 | <500 | >3,000 |
| 53 | >3,000 | >3,000 | >3,000 |
| 54 | >3,000 | >3,000 | >3,000 |
| 55 | 1,000-3,000 | >3,000 | >3,000 |
| 59 | >3,000 | >3,000 | >3,000 |
| 60 | >3,000 | >3,000 | >3,000 |
| 61 | >3,000 | >3,000 | >3,000 |
| 62 | >3,000 | >3,000 | >3,000 |
| 63 | >3,000 | >3,000 | >3,000 |
| 64 | >3,000 | >3,000 | >3,000 |
| 65 | >3,000 | >3,000 | >3,000 |
| 66 | >3,000 | >3,000 | >3,000 |
| 67 | <500 | <500 | 1,000-3,000 |
| 68 | <500 | 500-1,000 | 1,000-3,000 |
| 69 | <500 | 1,000-3,000 | >3,000 |
| 70 | <500 | <500 | >3,000 |
| 71 | >3,000 | >3,000 | >3,000 |
| 72 | 500-1,000 | <500 | >3,000 |
| 73 | 500-1,000 | 1,000-3,000 | >3,000 |
| 76 | <500 | 500-1,000 | >3,000 |
| 77 | >3,000 | >3,000 | >3,000 |
| 78 | <500 | <500 | >3,000 |
| 79 | <500 | <500 | 1,000-3,000 |
| 80 | <500 | 500-1,000 | 1,000-3,000 |
| 81 | <500 | <500 | >3,000 |
| 82 | <500 | <500 | >3,000 |
| 86 | <500 | 500-1,000 | 1,000-3,000 |
| 87 | <500 | <500 | >3,000 |
| 88 | >3,000 | >3,000 | >3,000 |
| 89 | <500 | <500 | >3,000 |
| 90 | 1,000-3,000 | >3,000 | >3,000 |
| 91 | 500-1,000 | 1,000-3,000 | >3,000 |
| 92 | >3,000 | >3,000 | >3,000 |
| 93 | 500-1,000 | 1,000-3,000 | >3,000 |
| 94 | 500-1,000 | 500-1,000 | >3,000 |
| 95 | <500 | <500 | >3,000 |
| 96 | 1,000-3,000 | >3,000 | >3,000 |
| 97 | 1,000-3,000 | 1,000-3,000 | >3,000 |
| 98 | >3,000 | >3,000 | >3,000 |
| 99 | 1,000-3,000 | >3,000 | >3,000 |
| 100 | <500 | <500 | 1,000-3,000 |
| 101 | >3,000 | >3,000 | >3,000 |
| 102 | 1,000-3,000 | 1,000-3,000 | 1,000-3,000 |
| 103 | 1,000-3,000 | 1,000-3,000 | 1,000-3,000 |
| 104 | <500 | 500-1,000 | >3,000 |
| 105 | 1,000-3,000 | 1,000-3,000 | >3,000 |
| 106 | 1,000-3,000 | 1,000-3,000 | >3,000 |
| 107 | >3,000 | 1,000-3,000 | >3,000 |
| 108 | >3,000 | >3,000 | >3,000 |
| 109 | 1,000-3,000 | 1,000-3,000 | 1,000-3,000 |
| 110 | 500-1,000 | 1,000-3,000 | 1,000-3,000 |
| 111 | 1,000-3,000 | 1,000-3,000 | >3,000 |
| 112 | 1,000-3,000 | 1,000-3,000 | >3,000 |
| 113 | 1,000-3,000 | 1,000-3,000 | 1,000-3,000 |
| 114 | 1,000-3,000 | 1,000-3,000 | 1,000-3,000 |
| 115 | 500-1,000 | 1,000-3,000 | 1,000-3,000 |
| 116 | >3,000 | >3,000 | 1,000-3,000 |
| 117 | >3,000 | >3,000 | >3,000 |
| 118 | <500 | <500 | 1,000-3,000 |
| 119 | 500-1,000 | 500-1,000 | >3,000 |
| 120 | >3,000 | >3,000 | >3,000 |
| 125 | >3,000 | >3,000 | >3,000 |
| 126 | 1,000-3,000 | >3,000 | 1,000-3,000 |
| 127 | 500-1,000 | 1,000-3,000 | 500-1,000 |
| 128 | 500-1,000 | >3,000 | <500 |
| 129 | 500-1,000 | >3,000 | 500-1,000 |
| 131 | 1,000-3,000 | >3,000 | >3,000 |
| 132 | 500-1,000 | 1,000-3,000 | 1,000-3,000 |
| 134 | >3,000 | >3,000 | >3,000 |
| 135 | 500-1,000 | 500-1,000 | 1,000-3,000 |
| 136 | <500 | 500-1,000 | 1,000-3,000 |
| 137 | 500-1,000 | <500 | 1,000-3,000 |
| 139 | 1,000-3,000 | 1,000-3,000 | >3,000 |
| 144 | 1,000-3,000 | 1,000-3,000 | <500 |
| 148 | >3,000 | >3,000 | >3,000 |
| 149 | >3,000 | >3,000 | >3,000 |
| 150 | >3,000 | 1,000-3,000 | >3,000 |
| 151 | 500-1,000 | 500-1,000 | 1,000-3,000 |
| 155 | >3,000 | >3,000 | >3,000 |
| 156 | >3,000 | >3,000 | >3,000 |
| 157 | >3,000 | >3,000 | >3,000 |
| 170 | >3,000 | >3,000 | >3,000 |
| 171 | >3,000 | >3,000 | >3,000 |
| 172 | >3,000 | >3,000 | >3,000 |
| 175 | >3,000 | >3,000 | >3,000 |
| 176 | 1,000-3,000 | 1,000-3,000 | >3,000 |
| 177 | >3,000 | >3,000 | >3,000 |
| 178 | 500-1,000 | 500-1,000 | >3,000 |
| 179 | 1,000-3,000 | 1,000-3,000 | >3,000 |
| 180 | 1,000-3,000 | 1,000-3,000 | >3,000 |
| 181 | >3,000 | >3,000 | >3,000 |
| 182 | >3,000 | >3,000 | >3,000 |
| 183 | >3,000 | >3,000 | >3,000 |
| 184 | >3,000 | >3,000 | >3,000 |
| 185 | >3,000 | >3,000 | >3,000 |
| 186 | >3,000 | >3,000 | >3,000 |
| 187 | <500 | 500-1,000 | >3,000 |
| 188 | <500 | <500 | 500-1,000 |
| 189 | <500 | <500 | 500-1,000 |
| 190 | 1,000-3,000 | 1,000-3,000 | >3,000 |
| 191 | >3,000 | >3,000 | >3,000 |
| 192 | 500-1,000 | 500-1,000 | >3,000 |
| 193 | <500 | 500-1,000 | >3,000 |
| 194 | <500 | <500 | <500 |
| 195 | 500-1,000 | >3,000 | >3,000 |
| 196 | >3,000 | >3,000 | >3,000 |
| 197 | 1,000-3,000 | 1,000-3,000 | >3,000 |
| 198 | 500-1,000 | <500 | >3,000 |
| 199 | >3,000 | >3,000 | >3,000 |
| 200 | <500 | <500 | <500 |
| 201 | <500 | 500-1,000 | >3,000 |
| 202 | >3,000 | >3,000 | |
| 203 | >3,000 | >3,000 | |
| 204 | >3,000 | >3,000 | |
| 205 | >3,000 | >3,000 | >3,000 |
| 206 | >3,000 | >3,000 | >3,000 |
| 207 | <500 | <500 | 500-1,000 |
| 208 | <500 | <500 | 1,000-3,000 |
| 209 | <500 | <500 | <500 |
| 210 | <500 | <500 | >3,000 |
| 211 | <500 | <500 | >3,000 |
| 212 | <500 | <500 | >3,000 |
| 213 | <500 | 500-1,000 | 1,000-3,000 |
| 214 | <500 | <500 | 1,000-3,000 |
| 215 | <500 | <500 | 1,000-3,000 |
| 216 | <500 | <500 | 1,000-3,000 |
| 217 | <500 | <500 | 1,000-3,000 |
| 218 | 1,000-3,000 | >3,000 | >3,000 |
| 219 | 500-1,000 | 500-1,000 | >3,000 |
| 220 | <500 | <500 | 1,000-3,000 |
| 221 | 500-1,000 | 1,000-3,000 | >3,000 |
| 222 | <500 | <500 | 1,000-3,000 |
| 223 | <500 | <500 | 1,000-3,000 |
| 224 | >3,000 | >3,000 | >3,000 |
| 225 | 1,000-3,000 | 1,000-3,000 | >3,000 |
| 226 | <500 | <500 | >3,000 |
| 227 | >3,000 | >3,000 | >3,000 |
| 228 | <500 | <500 | >3,000 |
| 229 | <500 | <500 | <500 |
| 230 | <500 | <500 | 1,000-3,000 |
| 231 | <500 | <500 | 1,000-3,000 |
| 232 | <500 | <500 | >3,000 |
| 233 | <500 | <500 | |
| 234 | 1,000-3,000 | 1,000-3,000 | >3,000 |
| 235 | <500 | <500 | 500-1,000 |
| 236 | <500 | <500 | 1,000-3,000 |
| 237 | <500 | 500-1,000 | >3,000 |
| 238 | <500 | <500 | >3,000 |
| 239 | <500 | <500 | >3,000 |
| 240 | <500 | <500 | 1,000-3,000 |
| 241 | <500 | <500 | 500-1,000 |

1. A compound of formula I, or a pharmaceutically acceptable salt thereof: 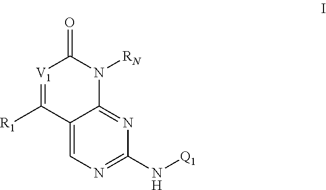
wherein:
R_{N} is selected from hydrogen, (1-6C)alkyl, (3-8C)cycloalkyl, (3-8C)cycloalkyl(1-4C)alkyl, aryl, aryl(1-4C)alkyl, carbon-linked heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in R_{N} is optionally substituted with one or more R_{NA} groups;
wherein R_{NA} is selected from halo, nitro, cyano, oxo or a group of the formula: 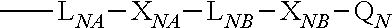
wherein:
L_{NA} is absent or (1-3C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)—N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-4C)alkyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —O—C(O)—N(R_{NB1})—, —N(R_{NB1})—C(O)—O—, —N(R_{NB2})—C(O)—NR_{NB1}—, SO₂N(R_{NB1})— or —N(R_{NB1})SO₂—, where R_{NB1} and R_{NB2} are independently selected from the group consisting of hydrogen or (1-4C)alkyl;
Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, aryl, aryl(1-2C)alkyl, heterocyclyl, heterocyclyl(1-2C)alkyl, heteroaryl, or heteroaryl(1-4C)alkyl;
and wherein Q_{N} is optionally substituted with one or more R_{NC} groups;
and each R_{NC} group present is independently selected from the group consisting of hydroxy, cyano, oxo, halogen, (1-2C)alkyl, (1-2C)alkoxy, (3-6C)cycloalkyl, —NR_{NC1}R_{NC2} or —C(O)—R_{NC1},
wherein R_{NC1} and R_{NC2} are each independently hydrogen or (1-2C)alkyl;
R₁ is selected from (2C)alkynyl or (2C)alkenyl;
V₁ is N or C—R₂;
R₂ is hydrogen, halo, cyano, or a group of the formula: 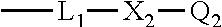
wherein:
L₁ is absent or (1-3C)alkylene;
X₂ is absent or is selected from the group consisting of —O—, —C(O)—, —NR₃ₐ—, —C(O)O, —O—C(O)—, —S(O)₀₋₂—, —C(O)—N(R₃ₐ)—, —N(R₃ₐ)—C(O)—, —O—C(O)—N(R₃ₐ)—, —N(R₃)—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-4C)alkyl;
Q₂ is selected from the group consisting of hydrogen, (1-6C)alkyl, (3-8C)cycloalkyl, (3-8C)cycloalkyl(1-4C)alkyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkyl-alkyl, aryl, arylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{3c} group, or any alkyl, cycloalkyl, or cycloalkyl-alkyl group present in a R_{3d} or R₃ₑ group, is optionally further substituted by one or more substituents independently selected from hydroxy, cyano, halogen, (1-2C)alkyl, —OR_{3f}, —NR_{3f}R_{3g} and —C(O)—R_{3f}, wherein R₃ₐ and R_{3g} are both independently selected from hydrogen and (1-2C)alkyl; and
Q₁ is selected from:
(i) a group of the formula: 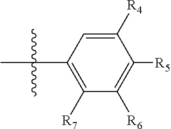
wherein:
R₄, R₆ and R₇ are each independently selected from hydrogen, halo, cyano, nitro, or a group of the formula: 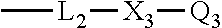
wherein:
L₂ is absent or (1-3C)alkylene;
X₃ is absent or is selected from the group consisting of —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂—, where R₄ₐ and R_{4b} are independently selected from the group consisting of hydrogen or (1-4C)alkyl;
Q₃ is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, —N(R₄ₑ)—S(O)₂R_{4d}, phenyl, 5 or 6-membered heteroaryl, or 4 to 6-membered heterocyclyl,
wherein R_{4d} and R₄ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{4c} group, or any alkyl, cycloalkyl or cycloalkyl-alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —C(O)OR_{4f}, —OC(O)R_{4f}, —C(O)NR_{4f}R_{4g}, —NR₄₉C(O)R_{4f}, —S(O)₀₋₂—R_{4f} and —C(O)—R_{4f}, wherein R_{4f} and R₄₉ are both independently selected from hydrogen and (1-2C)alkyl;
R₅ is:
a) hydrogen, halo, cyano;
b) a group of the formula: 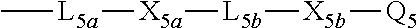
wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c}groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R₅₉ are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula 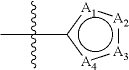
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkylalkyl, phenyl, benzyl, heterocyclyl, heterocyclylalkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 4 of A₁, A₂, A₃ and A₄ can be N;
(iii) a group of the formula: 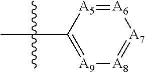
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that one or two of A₅, A₆, A₇, A₈ and A₉ can be N;
(iv) a group of the formula: 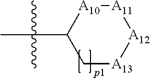
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula: 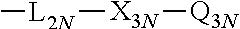
wherein:
L_{2N} is absent or (1-3C)alkylene;
X_{3N} is absent or is selected from the group consisting of:
(i) —C(O)—, —S(O)₀₋₂— or —C(O)—N(R₄ₐ)— when L₂ is absent; or
(ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
wherein R₄ₐ and R_{4b} are as defined above;
Q_{3N} is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is as defined above;
R_{5N} is:
a) hydrogen;
b) a group of the formula: 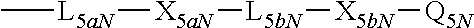
wherein:
L_{5aN} is absent or (1-3C)alkylene;
X_{5aN} is absent or is selected from the group consisting of:
(iii) —C(O)—, —S(O)₀₋₂—, —C(O)—N(R₅ₐ₁)— when L_{5aN} is absent; or
(iv) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-4C)alkylene;
X_{5bN} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-6C)alkyl, (3-8C)cycloalkyl, (3-8C)cycloalkyl(1-4C)alkyl, phenyl, phenyl(1-4C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
or R_{4N} and R_{5N}, or R_{5N} and R_{6N} are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
and with the proviso that one or two of A₁₀, A₁₁, A₁₂ and A₁₃ can be N;
(v) a group of the formula: 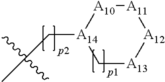
wherein:
p1, A₁₀, A₁₁, A₁₂, and A₁₃ are as defined above;
p2 is 1 to 6;
A₁₄ is C or N;
and with the proviso that one of A₁₀, A₁₁, A₁₂ and A₁₃ can be N;
or:
R_{N} and one of R₄ or R₅ are optionally linked to form a linker group L.

2. A compound according to claim 1, or a pharmaceutically acceptable salt thereof, wherein R_{N} is selected from hydrogen, (1-4C)alkyl, (3-6C)cycloalkyl, (3-6C)cycloalkyl(1-4C)alkyl, phenyl(1-4C)alkyl, carbon-linked 4- to 6-membered heterocyclyl, 4- to 6-membered heterocyclyl(1-4C)alkyl, 5- or 6-membered heteroaryl and [5- or 6-membered heteroaryl](1-4C)alkyl, and wherein any alkyl, cycloalkyl, cycloalkylalkyl, aryl, arylalkyl, heterocyclyl, heterocyclylalkyl, heteroaryl or heteroarylalkyl in R_{N} is optionally substituted with one or more R_{NA} groups, wherein R_{NA} is as defined in claim 1.

3. A compound according to claim 1 or claim 2, or a pharmaceutically acceptable salt thereof, wherein R_{N} is selected from (1-4C)alkyl, (3-6C)cycloalkyl, (3-6C)cycloalkyl(1-2C)alkyl, phenyl(1-2C)alkyl, carbon-linked 5- to 6-membered heterocyclyl, 5- to 6-membered heterocyclyl(1-2C)alkyl, 5- or 6-membered heteroaryl and [5- or 6-membered heteroaryl](1-2C)alkyl, and wherein any alkyl, cycloalkyl, cycloalkylalkyl, aryl, arylalkyl, heterocyclyl, heterocyclylalkyl, heteroaryl or heteroarylalkyl in R_{N} is optionally substituted with one or more R_{NA} groups, wherein R_{NA} is as defined in claim 1.

4. A compound according to any one of the preceding claims, or a pharmaceutically acceptable salt thereof, wherein R_{NA} is selected from halo, nitro, cyano, oxo or a group of the formula: 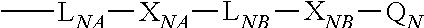
wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)—N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-2C)alkyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —O—C(O)—N(R_{NB1})—, —N(R_{NB1})—C(O)—O—, —N(R_{NB2})—C(O)—NR_{NB1}—, —SO₂N(R_{NB1})— or —N(R_{NB1})SO₂—, where R_{NB1} and R_{NB2} are independently selected from the group consisting of hydrogen or (1-2C)alkyl;
Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, phenyl, phenyl(1-2C)alkyl, 3- to 8-membered heterocyclyl, [3- to 8-membered heterocyclyl](1-2C)alkyl, 5- or 6-membered heteroaryl, or [5- or 6-membered heteroaryl](1-2C)alkyl;
and wherein Q_{N} is optionally substituted with one or more R_{NC} groups;
and each R_{NC} group present is independently selected from the group consisting of hydroxy, cyano, oxo, halogen, (1-2C)alkyl, (1-2C)alkoxy, (3-6C)cycloalkyl, —NR_{NC1}R_{NC2} or —C(O)—R_{NC1},
wherein R_{NC1} and R_{NC2} are each independently hydrogen or (1-2C)alkyl.

5. A compound according to any one of the preceding claims, or a pharmaceutically acceptable salt thereof, wherein R_{NA} is selected from halo, nitro, cyano, oxo or a group of the formula: 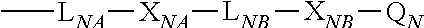
wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})—, —N(R_{NA1})—C(O)—, —O—C(O)—N(R_{NA1})—, —N(R_{NA1})—C(O)—O—, —N(R_{NA2})—C(O)—NR_{NA1}—, —SO₂N(R_{NA1})— or —N(R_{NA1})SO₂—, where R_{NA1} and R_{NA2} are independently selected from the group consisting of hydrogen or (1-2C)alkyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —O—C(O)—N(R_{NB1})—, —N(R_{NB1})—C(O)—O—, —N(R_{NB2})—C(O)—NR_{NB1}—, —SO₂N(R_{NB1})— or —N(R_{NB1})SO₂—, where R_{NB1} and R_{NB2} are independently selected from the group consisting of hydrogen or (1-2C)alkyl;
Q_{N} is selected from the group consisting of hydrogen, (1-6C)alkyl, (2-6C)alkenyl, (2-6C)alkynyl, (3-8C)cycloalkyl, (3-8C)cycloalkyl(1-2C)alkyl, phenyl, phenyl(1-2C)alkyl, 3- to 8-membered heterocyclyl, [3- to 8-membered heterocyclyl](1-2C)alkyl, 5- or 6-membered heteroaryl, or [5- or 6-membered heteroaryl](1-2C)alkyl.

6. A compound according to any one of the preceding claims, or a pharmaceutically acceptable salt thereof, wherein R_{NA} is selected from halo, nitro, cyano, oxo or a group of the formula: 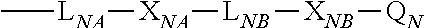
wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})— or —N(R_{NA1})—C(O)—, where R_{NA1} is hydrogen or methyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —SO₂N(R_{NB1})— or —N(R_{NB1})SO₂—, where R_{NB1} and R_{NB2} are independently selected from the group consisting of hydrogen or methyl;
Q_{N} is selected from the group consisting of hydrogen, (1-6C)alkyl, (2-6C)alkenyl, (3-6C)cycloalkyl, 3- to 6-membered heterocyclyl, [3- to 6-membered heterocyclyl](1-2C)alkyl, 5- or 6-membered heteroaryl, or [5- or 6-membered heteroaryl](1-2C)alkyl.

7. A compound according to any one of the preceding claims, or a pharmaceutically acceptable salt thereof, wherein R_{NA} is selected from halo, nitro, cyano, oxo or a group of the formula: 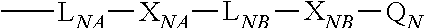
wherein:
L_{NA} is absent or (1-2C)alkylene;
X_{NA} is absent or is selected from the group consisting of —O—, —C(O)—, —NR_{NA1}—, —C(O)—O—, —O—C(O)—, —S(O)₀₋₂—, —C(O)—N(R_{NA1})— or —N(R_{NA1})—C(O)—, where R_{NA1} is hydrogen or methyl;
L_{NB} is absent or (1-4C)alkylene;
X_{NB} is absent or is selected from the group consisting of —O—, —C(O)—, —NR_{NB1}—, —C(O)—O—, —O—C(O)—, —S(O)₀₋₂—, —C(O)—N(R_{NB1})—, —N(R_{NB1})—C(O)—, —SO₂N(R_{NB1})— or —N(R_{NB1})SO₂—, where R_{NB1} and R_{NB2} are independently selected from the group consisting of hydrogen or methyl;
Q_{N} is selected from the group consisting of hydrogen, (1-6C)alkyl, (2-6C)alkenyl, (3-6C)cycloalkyl, 5- or 6-membered heteroaryl, or [5- or 6-membered heteroaryl](1-2C)alkyl.

8. A compound according to any one of the preceding claims, or a pharmaceutically acceptable salt thereof, wherein R₁ is selected from ethynyl.

9. A compound according to any one of the preceding claims, or a pharmaceutically acceptable salt thereof, wherein V₁ is N.

10. A compound according to any one of the preceding claims, or a pharmaceutically acceptable salt thereof, wherein V₁ is C—R₂.

11. A compound according to any one of the preceding claims, or a pharmaceutically acceptable salt thereof, wherein R₂ is hydrogen, halo, cyano, or a group of the formula 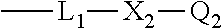
wherein:
L₁ is absent or (1-2C)alkylene;
X₂ is absent or is selected from the group consisting of —O—, —C(O)—, —NR₃ₐ—, —C(O)O, —O—C(O)—, —S(O)₀₋₂—, —C(O)—N(R₃ₐ)—, —N(R₃ₐ)—C(O)—, —O—C(O)—N(R₃ₐ)—, —N(R₃)—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 8-membered heterocyclyl, [3- to 8-membered heterocyclyl](1-2C)alkyl, 5- or 6-membered heteroaryl and [5- to 6-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen, (1-4C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{3c} group, or any alkyl, cycloalkyl, or cycloalkyl-alkyl group present in a R_{3d} or R₃ₑ group, is optionally further substituted by one or more substituents independently selected from hydroxy, cyano, halogen, (1-2C)alkyl, —OR_{3f}, —NR_{3f}R_{3g} and —C(O)—R_{3f}, wherein R₃ₐ and R_{3g} are both independently selected from hydrogen and (1-2C)alkyl;

12. A compound according to any one of the preceding claims, or a pharmaceutically acceptable salt thereof, wherein R₂ is hydrogen, cyano, or a group of the formula 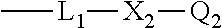
wherein:
L₁ is absent or (1-2C)alkylene;
X₂ is absent or is selected from the group consisting of —O—, —C(O)—, —NR₃ₐ—, —C(O)—, —O—C(O)—, —S(O)₀₋₂—, —C(O)—N(R₃ₐ)—, —N(R₃ₐ)—C(O)—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- or 6-membered heteroaryl and [5- to 6-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl;
and wherein any alkyl, alkoxy, cycloalkyl, cycloalkyl-alkyl, phenyl, heteroaryl or heterocyclyl group present in a R_{3c} group is optionally further substituted by one or more substituents independently selected from hydroxy, cyano, halogen, (1-2C)alkyl, or —OR_{3f}, wherein R_{3f} is selected from hydrogen and (1-2C)alkyl.

13. A compound according to any one of the preceding claims, or a pharmaceutically acceptable salt thereof, wherein R₂ is hydrogen, cyano, or a group of the formula 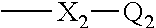
wherein:
X₂ is absent or is selected from the group consisting of —NR₃ₐ—, —C(O)—O—, —C(O)—N(R₃ₐ)— or —N(R₃ₐ)—C(O)—, where R₃ₐ is selected from the group consisting of hydrogen or methyl;
Q₂ is selected from the group consisting of hydrogen, (1-3C)alkyl, (3-6C)cycloalkyl, phenyl, phenyl(1-4C)alkyl, 5- or 6-membered heteroaryl and [5- to 6-membered heteroaryl](1-2C)alkyl, wherein any alkyl, cycloalkyl, phenyl, phenylalkyl, heteroaryl or heteroaryl-alkyl in Q₂ is optionally substituted with one or more R_{3c} groups; and
each R_{3c} group present is independently selected from the group consisting of hydroxy, cyano, halogen, (1-4C)alkyl, (1-4C)alkoxy, or 5 or 6-membered heteroaryl.

14. A compound according to any one of the preceding claims, or a pharmaceutically acceptable salt thereof, wherein Q₁ is selected from:
(i) a group of the formula: 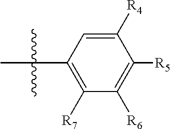
wherein:
R₄ and R₆ are each independently selected from hydrogen, or a group of the formula: 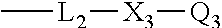
wherein:
L₂ is absent or (1-3C)alkylene;
X₃ is absent or is selected from the group consisting of —O—, —NR₄ₐ—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, phenyl, 3 to 8-membered heterocyclyl, or 5 or 6-membered heteroaryl, wherein any alkyl, cycloalkyl, cycloalkylalkyl, phenyl, heterocyclyl, or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
R₅ is:
a) hydrogen, halo, cyano; or
b) a group of the formula: 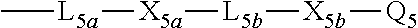
wherein:
L₅ₐ is absent or (1-3C)alkylene;
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R₅₉ are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 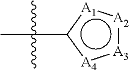
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{N};
(iii) 1 to 3 of A₁, A₂, A₃ and A₄ can be N;
(iii) a group of the formula: 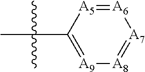
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 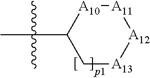
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula: 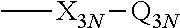
wherein:
X_{3N} is absent or is selected from the group consisting of: —C(O)—, —S(O)₀₋₂— or —C(O)—N(R₄ₐ)—;
wherein R₄ₐ is as defined above;
Q_{3N} is selected from the group consisting of hydrogen, (1-4C)alkyl, (3-6C)cycloalkyl, (3-6C)cycloalkyl(1-2C)alkyl, heterocyclyl, heteroaryl and,
wherein any alkyl, cycloalkyl, cycloalkylalkyl, heterocyclyl or heteroaryl in Q₃ is optionally substituted with one or more R_{4c} groups; and
each R₄ₑ group present is as defined above;
R_{5N} is:
a) hydrogen;
b) a group of the formula: 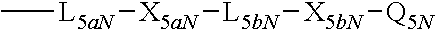
wherein:
L_{5aN} is absent or (1-3C)alkylene;
X_{5aN} is absent or is selected from the group consisting of:
(iii) —C(O)—, —S(O)₀₋₂—, —C(O)—N(R₅ₐ₁)— when L_{5aN} is absent; or
(iv) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 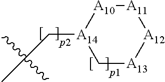
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or:
R_{N} and one of R₄ or R₅ are optionally linked to form a linker group L.

15. A compound according to any one of the preceding claims, or a pharmaceutically acceptable salt thereof, wherein Q₁ is selected from:
(i) a group of the formula: 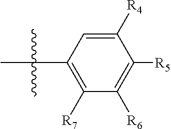
wherein:
R₄ and R₆ are each independently selected from hydrogen, or a group of the formula: 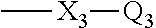
wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, phenyl, 3 to 8-membered heterocyclyl, or 5 or 6-membered heteroaryl, wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-2C)alkyl;
R₅ is:
a) hydrogen, halo, cyano; or
b) a group of the formula: 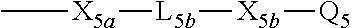
wherein:
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R₅₉ are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 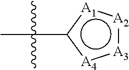
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 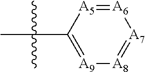
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 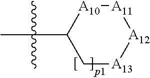
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
a) hydrogen;
b) a group of the formula: 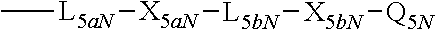
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 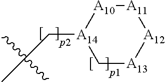
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or:
R_{N} and R₄ are linked to form a linker group L.

16. A compound according to any one of the preceding claims, or a pharmaceutically acceptable salt thereof, wherein:
(1) Q₁ is selected from:
(i) a group of the formula: 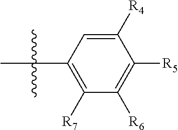
wherein:
R₄, R₆ and R₇ are each independently selected from hydrogen, halo, cyano, or a group of the formula: 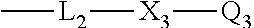
wherein:
L₂ is absent or (1-3C)alkylene;
X₃ is absent or is selected from the group consisting of —O—, —NR₄ₐ—, —C(O)—O—, —O—C(O)—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclyl-alkyl, heteroaryl or heteroaryl-alkyl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
and wherein any alkyl, alkoxy, cycloalkyl, cycloalkyl-alkyl group present in a R_{4c} group, or any alkyl group present in a R_{4d} or R₄ₑ group, is optionally further substituted by one or more substituents independently selected from hydroxy, cyano, halogen, R_{4f}, —OR_{4f}, —NR_{4f}R_{4g} —C(O)OR_{4f}, —OC(O)R_{4f}, —C(O)NR_{4f}R_{4g}, —NR₄₉C(O)R_{4f}, —S(O)₀₋₂—R_{4f} and —C(O)—R_{4f}, wherein R_{4f} and R_{4g} are both independently selected from hydrogen or (1-2C)alkyl;
R₅ is:
a) hydrogen, halo, cyano; or
b) a group of the formula: 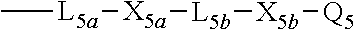
wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c}groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R₅₉ are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 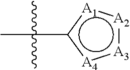
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 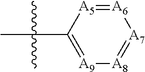
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 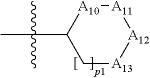
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula: 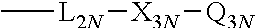
wherein:
L_{2N} is absent or (1-3C)alkylene;
X_{3N} is absent or is selected from the group consisting of:
(i) —C(O)—, —S(O)₀₋₂— or —C(O)—N(R₄ₐ)— when L₂ is absent; or
(ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
wherein R₄ₐ and R_{4b} are as defined above;
Q_{3N} is selected from the group consisting of hydrogen, (1-6C)alkyl, (3-6C)cycloalkyl, (3-6C)cycloalkyl(1-2C)alkyl, heterocyclyl, heterocyclyl(1-2C)alkyl, heteroaryl and heteroaryl(1-2C)alkyl, wherein any alkyl, cycloalkyl, cycloalkylalkyl, heterocyclyl, heterocyclylalkyl, heteroaryl or heteroarylalkyl in Q₃ is optionally substituted with one or more R_{4c} groups; and
each R_{4c} group present is as defined above;
R_{5N} is:
a) hydrogen;
b) a group of the formula: 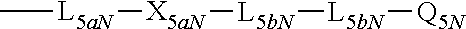
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5bl}—, —C(O)—O—, —O—C(O)—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 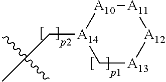
wherein:
p1, A₁₀, A₁₁, A₁₂, and A₁₃ are as defined above;
p2 is 1 to 5;
A₁₄ is C or N;
and with the proviso that one of A₁₀, A₁₁, A₁₂ and A₁₃ can be N;
or:
R_{N} and R₄ are linked to form a linker group L;
(2) Q₁ is selected from:
(i) a group of the formula: 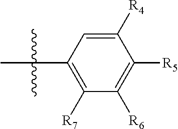
wherein:
R₄, R₆ and R₇ are each independently selected from hydrogen, or a group of the formula: 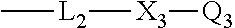
wherein:
L₂ is absent or (1-3C)alkylene;
X₃ is absent or is selected from the group consisting of —O—, —NR₄ₐ—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-8C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl, or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
and wherein any alkyl, alkoxy, cycloalkyl, cycloalkyl-alkyl, phenyl, heteroaryl or heterocyclyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f},
wherein R_{4f} and R₄₉ are both independently selected from hydrogen and (1-2C)alkyl;
R₅ is:
a) hydrogen, halo, cyano; or
b) a group of the formula: 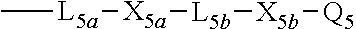
wherein:
L₅ₐ is absent or (1-3C)alkylene;
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R₅₉ are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 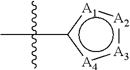
wherein:
A₁ is selected from N, NR_{1N}, O, S or CR₇;
A₂ is selected from N, NR_{1N}, O, S or CR₄;
A₃ is selected from N, NR_{N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 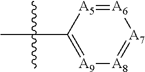
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 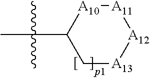
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula: 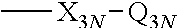
wherein:
X_{3N} is absent or is selected from the group consisting of: —C(O)—, —S(O)₀₋₂— or —C(O)—N(R₄ₐ)—;
wherein R₄ₐ is as defined above;
Q_{3N} is selected from the group consisting of hydrogen, (1-4C)alkyl, (3-6C)cycloalkyl, (3-6C)cycloalkyl(1-2C)alkyl, heterocyclyl, heteroaryl and,
wherein any alkyl, cycloalkyl, cycloalkylalkyl, heterocyclyl or heteroaryl in Q₃ is optionally substituted with one or more R_{4c} groups; and
each R_{4c} group present is as defined above;
R_{5N} is:
a) hydrogen;
b) a group of the formula: 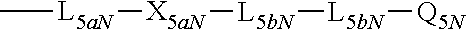
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 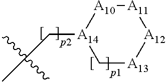
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or:
R_{N} and R₄ are linked to form a linker group L;
(3) Q₁ is selected from:
(i) a group of the formula: 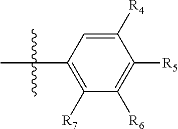
wherein:
R₄, R₆ and R₇ are each independently selected from hydrogen, or a group of the formula: 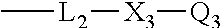
wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-2C)alkyl;
and wherein any alkyl, alkoxy, cycloalkyl, cycloalkyl-alkyl, phenyl, heteroaryl or heterocyclyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f},
wherein R_{4f} and R₄₉ are both independently selected from hydrogen and (1-2C)alkyl;
R₅ is:
a) hydrogen, halo, cyano; or
b) a group of the formula: 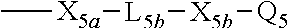
wherein:
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R₅₉ are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 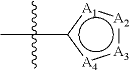
wherein:
A₁ is selected from N, NR_{1N}, O, S or CR₇;
A₂ is selected from N, NR_{1N}, O, S or CR₄;
A₃ is selected from N, NR_{1N}, O, S or CR;
A₄ is selected from N, NR₁N, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 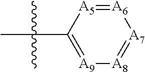
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 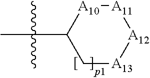
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
a) hydrogen;
b) a group of the formula: 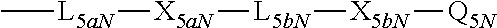
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 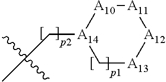
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or:
R_{N} and R₄ are linked to form a linker group L;
(4) Q₁ is selected from:
(i) a group of the formula: 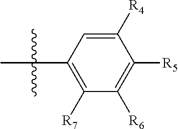
wherein:
R₄, R₆ and R₇ are each independently selected from hydrogen, or a group of the formula: 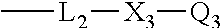
wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
and wherein any alkyl, alkoxy, cycloalkyl, cycloalkyl-alkyl, phenyl, heteroaryl or heterocyclyl group present in a R_{4c} group, or any methyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f},
wherein R_{4f} and R₄₉ are both independently selected from hydrogen and (1-2C)alkyl;
R₅ is:
a) hydrogen, halo, cyano; or
b) a group of the formula: 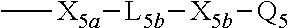
wherein:
X₅ₐ is absent or is selected from the group consisting of —O—, —C(O)—, —NR₅ₐ₁— or —N(R₅ₐ)—C(O)—, where R₅ₐ₁ is independently selected from the group consisting of hydrogen or methyl;
L_{5b} is absent or (1-4C)alkylene;
X_{5b} is absent or is selected from the group consisting of —O—, —C(O)— or —NR_{5b1}—, where R_{5b1} and R_{5b2} are independently selected from the group consisting of hydrogen or (1-2C)alkyl;
Q₅ is selected from the group consisting of hydrogen, (1-6C)alkyl or 3 to 8-membered heterocyclyl and wherein Q₅ is optionally substituted with one or more R_{5c} groups;
and each R_{5c} group present is independently selected from the group consisting of hydroxy, cyano, halogen, methyl, methoxy
(ii) a group of the formula: 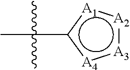
wherein:
A₁ is selected from N, NR_{1N}, O, S or CR₇;
A₂ is selected from N, NR_{N}, O, S or CR₄;
A₃ is selected from N, NR_{1N}, O, S or CR;
A₄ is selected from N, NR₁N, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 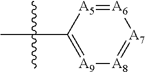
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 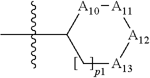
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
a) hydrogen;
b) a group of the formula: 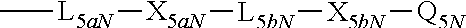
wherein:
L_{5aN} is absent or (1-3C)alkylene;
X_{5aN} is absent or is selected from the group consisting of:
(i) —C(O)—, —S(O)₀₋₂— or —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—, where R_{5bl} and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 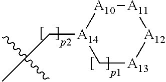
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N:
or:
R_{N} and R₄ are linked to form a linker group L;
(5) Q₁ is selected from:
(i) a group of the formula: 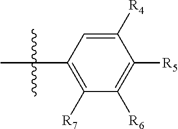
wherein:
two of R₄, R₆ and R₇ are hydrogen, and the other is hydrogen or a group of the formula: 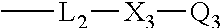
wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
and wherein any methyl, methoxy or cycloalkyl, group present in a R₄ₑgroup, or any methyl group present in a R_{4d} or R₄ₑ group, is optionally further substituted by one or more substituents independently selected from hydroxy, cyano, halogen, —OR_{4f}, —NR_{4f}R₄₉, —S(O)₀₋₂—R_{4f} and —C(O)—R_{4f}, wherein R_{4f} and R₄₉ are both independently selected from hydrogen and (1-2C)alkyl;
R₅ is hydrogen or a group of the formula: 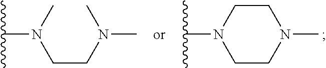
and
(ii) a group of the formula: 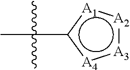
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 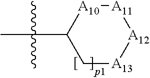
wherein:
p1 is 0 or 1;
A₁₀ is selected from CH₂ or CHR₇;
A₁₁ is selected from OH₂ or CHR₄;
A₁₂ is selected from NR_{5N}, OH₂ or CHR;
A₁₃ is selected from OH₂ or CHR₆;
R₄, R₅, R₆ and R₇ are as defined above;
R_{5N} is:
a) hydrogen;
b) a group of the formula: 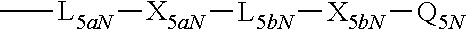
wherein:
L_{5aN} is absent or (1-3C)alkylene;
X_{5aN} is absent or is selected from the group consisting of:
(i) —C(O)—, —S(O)₀₋₂— or —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(iv) a group of the formula: 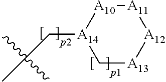
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N;
or:
R_{N} and R₄ are linked to form a linker group L.

17. A compound according to any one of the preceding claims, or a pharmaceutically acceptable salt thereof, wherein L is a linker group that separates the N atom to which R_{N} is attached and the carbon atom to which R₄ is attached by 5, 6 or 7 atoms, or 6, 7 or 8 bond lengths.

18. A compound according to any one of the preceding claims, or a pharmaceutically acceptable salt thereof, wherein L is a linker group that separates the N atom to which R_{N} is attached and the carbon atom to which R₄ is attached by 5 or 6 atoms, or 6 or 7 bond lengths.

19. A compound according to any one of the preceding claims, or a pharmaceutically acceptable salt thereof, wherein L is a linker group of the formula: 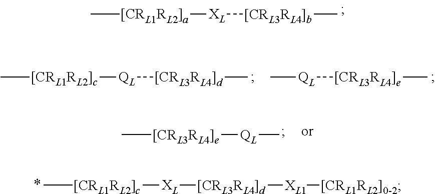
wherein:
R_{L1}, R_{L2}, R_{L3} and R_{L4} are, at each occurrence, independently selected from hydrogen or (1-2C)alkyl;
X_{L} or X_{L1} is selected from —R_{L5}C=CR_{L6}—, ethynylene, —O—, —N(R_{LN})—, —C(O)—N(R_{LN})—, —N(R_{LN})—C(O)—, —C(O)—O—, —O—C(O)—, —S(O)₀₋₂—, —O—C(O)—N(R_{LN})—, —N(R_{LN})—C(O)—O—, —N(R_{LN1})—C(O)—N(R_{LN})—, —SO₂N(R_{LN})— or —N(R_{LN})SO₂—, wherein R_{L5} and R_{L6} are, at each occurrence, independently selected from hydrogen or (1-2C)alkyl, and R_{LN} and R_{LN1} are each independently selected from hydrogen or (1-2C)alkyl;
Q_{L} is selected from a (4-6C)cycloalkyl, a 4 to 7 membered heterocyclyl or a 5 or 6-membered heteroaryl ring;
Q_{L1} is selected from a (4-6C)cycloalkyl, a carbon-linked 4 to 7 membered heterocyclyl or a carbon-linked 5 or 6-membered heteroaryl ring;
a is 1-6;
b is 0-6;
c is 1-6;
d is 0-6; and
e is 1-6;
and wherein Q_{L} and Q_{L1} are optionally substituted by halo, cyan, hydroxy, (1-2C)alkyl, (1-2C)alkoxy, amino, (1-2C)alkylamino, or [di-(1-2C)alkyl]amino.

20. A compound according to any one of the preceding claims, or a pharmaceutically acceptable salt thereof, wherein L is a linker group of the formula: 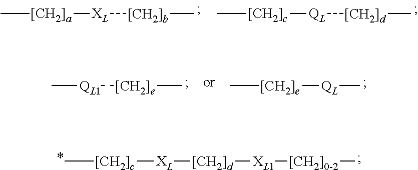
wherein:
X_{L} or X_{L1} is selected from —R_{L5}C═CR_{L6}—, ethynylene, —O—, —N(R_{LN})—, —C(O)—N(R_{LN})—, —N(R_{LN})—C(O)—, —C(O)—O—, —O—C(O)—, —S(O)₀₋₂—, —O—C(O)—N(R_{LN})—, —N(R_{LN})—C(O)—O—, —N(R_{LN1})—C(O)—N(R_{LN})—, —SO₂N(R_{LN})— or —N(R_{LN})SO₂—, wherein R_{L5} and R_{L6} are, at each occurrence, independently selected from hydrogen or (1-2C)alkyl, and R_{LN} and R_{LN}1 are each independently selected from hydrogen or (1-2C)alkyl;
Q_{L} is selected from a (4-6C)cycloalkyl, a 4 to 7 membered heterocyclyl or a 5 or 6-membered heteroaryl ring;
Q_{L1} is selected from a (4-6C)cycloalkyl, a carbon-linked 4 to 7 membered heterocyclyl or a carbon-linked 5 or 6-membered heteroaryl ring;
a is 1-5;
b is 0-3;
c is 1-3;
d is 0-3; and
e is 1-4;
and wherein Q_{L} and Q_{L1} are optionally substituted by halo, cyan, hydroxy, (1-2C)alkyl, (1-2C)alkoxy, amino, (1-2C)alkylamino, or [di-(1-2C)alkyl]amino.

21. A compound according to any one of the preceding claims, or a pharmaceutically acceptable salt thereof, wherein L is a linker group of the formula: 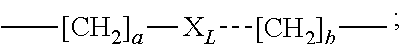
wherein:
X_{L} or X_{L1} is selected from —R_{L5}C=CR_{L6}—, ethynylene, —O—, —N(R_{LN})—, —C(O)—N(R_{LN})—, —N(R_{LN})—C(O)—, —S(O)₀₋₂—, —SO₂N(R_{LN})— or —N(R_{LN})SO₂—, wherein R_{L5} and R_{L6} are, at each occurrence, independently selected from hydrogen or methyl, and R_{LN} and R_{LN}1 are each independently selected from hydrogen or methyl;
Q_{L} is selected from a (5-6C)cycloalkyl, a 5 to 7 membered heterocyclyl or a 5 or 6-membered heteroaryl ring;
Q_{L1} is selected from a (4-6C)cycloalkyl, a carbon-linked 5 to 7 membered heterocyclyl or a carbon-linked 5 or 6-membered heteroaryl ring;
a is 1-2;
b is 0-2;
c is 1-2;
d is 0-2; and
e is 1-3.

22. A compound according to any one of the preceding claims, or a pharmaceutically acceptable salt thereof, wherein L is:
(i) a linker group of the formula: 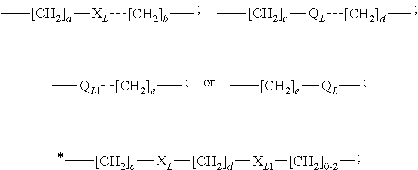
wherein:
X_{L} or X_{L1} is selected from —R_{L5}C=CR_{L6}—, —O—, —N(R_{LN})—, —C(O)—N(R_{LN})— or —N(R_{LN})—C(O)—, wherein R_{L5} and R_{L6} are, at each occurrence, independently selected from hydrogen or methyl, and R_{LN} and R_{LN}1 are each independently selected from hydrogen or methyl;
Q_{L} is selected from a (5-6C)cycloalkyl, a 5 to 7 membered heterocyclyl or a 5 or 6-membered heteroaryl ring;
Q_{L1} is selected from a (4-6C)cycloalkyl, a carbon-linked 5 to 7 membered heterocyclyl or a carbon-linked 5 or 6-membered heteroaryl ring;
a is 1-2:
b is 0-2;
c is 1-2;
d is 0-2; and
e is 1-3; or
(ii) a linker group of the formula: 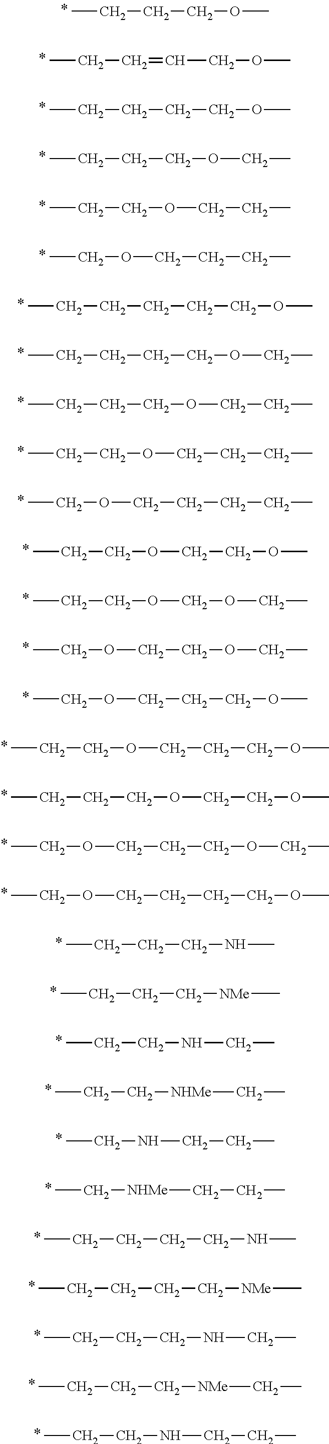 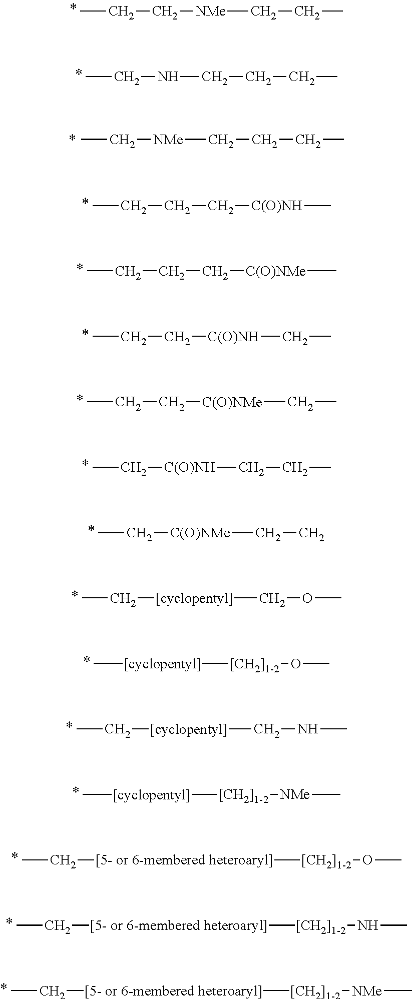
wherein:
* denotes the point of attachment to the N atom in formula I; or
(iii) a linker group of the formula: 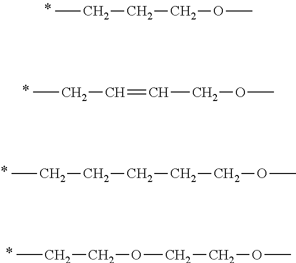
wherein:
* denotes the point of attachment to the N atom in formula I.

23. A compound according to claim 1, wherein the compound is a compound has one of the following structural formulae: 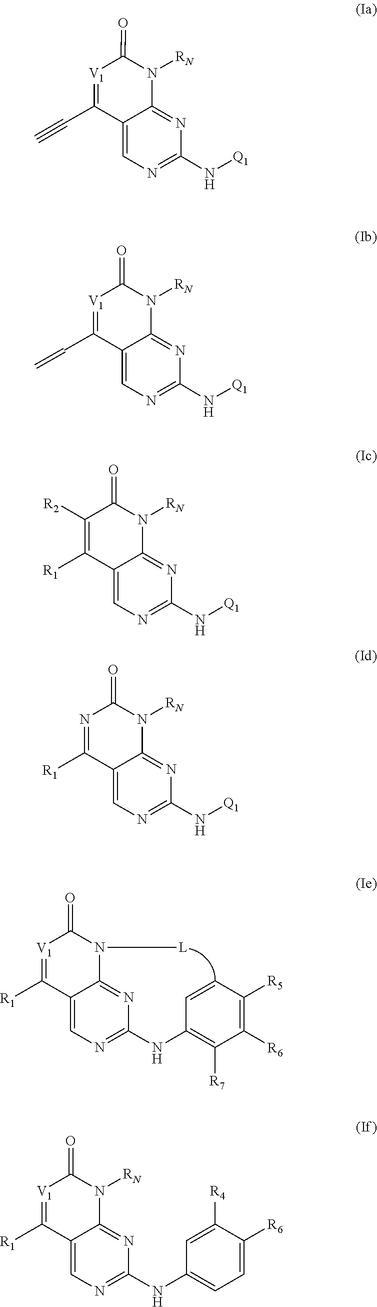
wherein R₁, R_{N}, V₁, Q₁, L, R₄ and R₅ are as defined in any one of the preceding claims.

24. A compound according to any one of the preceding claims, or a pharmaceutically acceptable salt thereof, wherein the compound is selected from any one of the following:
5-ethynyl-2-((2-methoxyphenyl)amino)-8-phenylpyrido[2,3-d]pyrimidin-7(8H)-one
N-(2-((2-methoxyphenyl)amino)-7-oxo-8-phenyl-7,8-dihydropyrido[2,3-d]pyrimidin-5-yl)acrylamide
5-ethynyl-2-((2-methoxyphenyl)amino)-8-methylpyrido[2,3-d]pyrimidin-7(8H)-one
8-cyclopentyl-5-ethynyl-2-((2-methoxyphenyl)amino)pyrido[2,3-d]pyrimidin-7(8H)-one
8-(2,4-dimethoxyphenyl)-5-ethynyl-2-((2-methoxyphenyl)amino)pyrido[2,3-d]pyrimidin-7(8H)-one
N-(4-((5-ethynyl-7-oxo-8-phenyl-7,8-dihydropyrido[2,3-d]pyrimidin-2-yl)amino)-3-methoxyphenyl)-2-methoxy-N-methylacetamide
5-ethynyl-2-[(2-methoxyphenyl)amino]-N-methyl-7-oxo-8-phenylpyrido[2,3-d]pyrimidine-6-carboxamide
5-ethynyl-2-((2-methoxyphenyl)amino)-7-oxo-8-phenyl-7,8-dihydropyrido[2,3-d]pyrimidine-6-carbonitrile
5-ethynyl-2-((2-methoxyphenyl)amino)-N,N,8-trimethyl-7-oxo-7,8-dihydropyrido[2,3-d]pyrimidine-6-carboxamide
5-ethynyl-8-isopropyl-2-((2-methoxyphenyl)amino)pyrido[2,3-d]pyrimidin-7(8H)-one
5-ethynyl-8-(4-methoxyphenyl)-2-((2-methoxyphenyl)amino)pyrido[2,3-d]pyrimidin-7(8H)-one
2-((2-methoxyphenyl)amino)-8-phenyl-5-vinylpyrido[2,3-d]pyrimidin-7(8H)-one
N-(3-(5-ethynyl-2-((2-methoxyphenyl)amino)-7-oxopyrido[2,3-d]pyrimidin-8(7H)-yl)phenyl)acetamide
N-(3-(5-ethynyl-2-((2-methoxyphenyl)amino)-7-oxopyrido[2,3-d]pyrimidin-8(7H)-yl)phenyl)methanesulfonamide
2-((2-methoxyphenyl)amino)-7-oxo-8-phenyl-5-vinyl-7,8-dihydropyrido[2,3-d]pyrimidine-6-carbonitrile
8-(2,4-dimethoxyphenyl)-2-((2-methoxyphenyl)amino)-5-vinylpyrido[2,3-d]pyrimidin-7(8H)-one
6-(dimethylamino)-5-ethynyl-2-((2-methoxyphenyl)amino)-8-phenylpyrido[2,3-d]pyrimidin-7(8H)-one
8-(2,4-dimethoxyphenyl)-2-((2-methoxyphenyl)amino)-7-oxo-5-vinyl-7,8-dihydropyrido[2,3-d]pyrimidine-6-carbonitrile
6-amino-5-ethynyl-2-((2-methoxyphenyl)amino)-8-phenylpyrido[2,3-d]pyrimidin-7(8H)-one
N-(5-ethynyl-2-((2-methoxyphenyl)amino)-7-oxo-8-phenyl-7,8-dihydropyrido[2,3-d]pyrimidin-6-yl)acetamide
N-(3-(5-ethynyl-2-((2-methoxyphenyl)amino)-7-oxopyrido[2,3-d]pyrimidin-8(7H)-yl)phenyl)acrylamide
5-ethynyl-8-phenyl-2-(phenylamino)pyrido[2,3-d]pyrimidin-7(8H)-one
6-(2-chlorophenyl)-2-((4-((2-(dimethylamino)ethyl)(methyl)amino)-2-methoxyphenyl)amino)-5-ethynyl-8-methylpyrido[2,3-d]pyrimidin-7(8H)-one
2-((4-((2-(dimethylamino)ethyl)(methyl)amino)-2-methoxyphenyl)amino)-5-ethynyl-8-methyl-6-phenylpyrido[2,3-d]pyrimidin-7(8H)-one
2-((4-((2-(dimethylamino)ethyl)(methyl)amino)-2-methoxyphenyl)amino)-5-ethynyl-8-methylpyrido[2,3-d]pyrimidin-7(8H)-one
6-benzyl-2-((4-((2-(dimethylamino)ethyl)(methyl)amino)-2-methoxyphenyl)amino)-5-ethynyl-8-methylpyrido[2,3-d]pyrimidin-7(8H)-one
4-ethynyl-7-((2-methoxyphenyl)amino)-1-phenylpyrimido[4,5-d]pyrimidin-2(1H)-one
2-((4-((2-(dimethylamino)ethyl)(methyl)amino)phenyl)amino)-5-ethynyl-8-phenylpyrido[2,3-d]pyrimidin-7(8H)-one
8-(2-methoxyphenyl)-2-((2-methoxyphenyl)amino)-5-vinylpyrido[2,3-d]pyrimidin-7(8H)-one
5-ethynyl-2-((2-methoxy-4-morpholinophenyl)amino)-8-phenylpyrido[2,3-d]pyrimidin-7(8H)-one
6-(2-chlorophenyl)-5-ethynyl-2-((2-methoxyphenyl)amino)-8-methylpyrido[2,3-d]pyrimidin-7(8H)-one
6-(2-chlorophenyl)-2-((2-methoxyphenyl)amino)-8-methyl-5-vinylpyrido[2,3-d]pyrimidin-7(8H)-one
2-((2-methoxyphenyl)amino)-8-methyl-6-phenyl-5-vinylpyrido[2,3-d]pyrimidin-7(8H)-one
6-(2-chlorophenyl)-5-ethynyl-8-methyl-2-(phenylamino)pyrido[2,3-d]pyrimidin-7(8H)-one
6-(2,6-dichlorophenyl)-2-((2-methoxyphenyl)amino)-8-methyl-5-vinylpyrido[2,3-d]pyrimidin-7(8H)-one
6-(2,6-dichlorophenyl)-5-ethynyl-2-((3-(hydroxymethyl)phenyl)amino)-8-methylpyrido[2,3-d]pyrimidin-7(8H)-one
6-(2,6-dichlorophenyl)-2-((3-(hydroxymethyl)phenyl)amino)-8-methyl-5-vinylpyrido[2,3-d]pyrimidin-7(8H)-one
6-(2-chlorophenyl)-2-((4-((2-(dimethylamino)ethyl)(methyl)amino)phenyl)amino)-5-ethynyl-8-methylpyrido[2,3-d]pyrimidin-7(8H)-one
6-(2-chlorophenyl)-2-((4-((2-(dimethylamino)ethyl)(methyl)amino)phenyl)amino)-8-methyl-5-vinylpyrido[2,3-d]pyrimidin-7(8H)-one
6-benzyl-2-((4-((2-(dimethylamino)ethyl)(methyl)amino)phenyl)amino)-8-methyl-5-vinylpyrido[2,3-d]pyrimidin-7(8H)-one
6-(2,6-dichlorophenyl)-2-((4-((2-(dimethylamino)ethyl)(methyl)amino)phenyl)amino)-8-methyl-5-vinylpyrido[2,3-d]pyrimidin-7(8H)-one
6-(2-chlorophenyl)-2-((3-methoxyphenyl)amino)-8-methyl-5-vinylpyrido[2,3-d]pyrimidin-7(8H)-one
6-(2-chlorophenyl)-2-((3-(hydroxymethyl)phenyl)amino)-8-methyl-5-vinylpyrido[2,3-d]pyrimidin-7(8H)-one
N-(3-(5-ethynyl-2-((2-methoxyphenyl)amino)-7-oxopyrido[2,3-d]pyrimidin-8(7H)-yl)phenyl)cyclopropanesulfonamide
N-(4-((5-ethynyl-7-oxo-8-phenyl-7,8-dihydropyrido[2,3-d]pyrimidin-2-yl)amino)-3-methoxyphenyl)-3-methoxy-N-methylpropanamide
6-(2-chlorophenyl)-5-ethynyl-2-((2-methoxy-4-(4-methylpiperazin-1-yl)phenyl)amino)-8-methylpyrido[2,3-d]pyrimidin-7(8H)-one
6-(2-chlorophenyl)-5-ethynyl-2-((3-(hydroxymethyl)phenyl)amino)-8-methylpyrido[2,3-d]pyrimidin-7(8H)-one
6-(2-chlorophenyl)-2-((2-methoxy-4-(4-methylpiperazin-1-yl)phenyl)amino)-8-methyl-5-vinylpyrido[2,3-d]pyrimidin-7(8H)-one
N-(2-((2-methoxyphenyl)amino)-7-oxo-8-phenyl-5-vinyl-7,8-dihydropyrido[2,3-d]pyrimidin-6-yl)acetamide
6-(2,6-dichlorophenyl)-2-((4-((2-(dimethylamino)ethyl)(methyl)amino)phenyl)amino)-5-ethynyl-8-methylpyrido[2,3-d]pyrimidin-7(8H)-one
6-(2-chlorophenyl)-5-ethynyl-8-methyl-2-((4-(4-methylpiperazin-1-yl)phenyl)amino)pyrido[2,3-d]pyrimidin-7(8H)-one
6-(2-chlorophenyl)-2-((4-(2-(dimethylamino)ethoxy)phenyl)amino)-5-ethynyl-8-methylpyrido[2,3-d]pyrimidin-7(8H)-one
6-(2-chlorophenyl)-5-ethynyl-2-((3-methoxyphenyl)amino)-8-methylpyrido[2,3-d]pyrimidin-7(8H)-one
6-(2-chlorophenyl)-8-methyl-2-((4-(4-methylpiperazin-1-yl)phenyl)amino)-5-vinylpyrido[2,3-d]pyrimidin-7(8H)-one
6-(2-chlorophenyl)-2-((4-(2-(dimethylamino)ethoxy)phenyl)amino)-8-methyl-5-vinylpyrido[2,3-d]pyrimidin-7(8H)-one
2-amino-6-(2-chlorophenyl)-8-methyl-5-vinylpyrido[2,3-d]pyrimidin-7(8H)-one
4-ethynyl-7-((2-methoxyphenyl)amino)-1-phenyl-1,6-naphthyridin-2(1H)-one
5-ethynyl-2-((4-(4-methylpiperazin-1-yl)phenyl)amino)-8-phenylpyrido[2,3-d]pyrimidin-7(8H)-one
(R)-2-((4-((2-(dimethylamino)ethyl)(methyl)amino)phenyl)amino)-5-ethynyl-8-(1-propionylpiperidin-3-yl)pyrido[2,3-d]pyrimidin-7(8H)-one
6-(2-chlorophenyl)-5-ethynyl-8-methyl-2-((4-(4-methylpiperazin-1-yl)butyl)amino)pyrido[2,3-d]pyrimidin-7(8H)-one
6-(2-chlorophenyl)-8-methyl-2-((4-(4-methylpiperazin-1-yl)butyl)amino)-5-vinylpyrido[2,3-d]pyrimidin-7(8H)-one
5-ethynyl-8-methyl-2-((4-(4-methylpiperazin-1-yl)phenyl)amino)pyrido[2,3-d]pyrimidin-7(8H)-one
2-((4-(2-(dimethylamino)ethoxy)phenyl)amino)-5-ethynyl-8-methylpyrido[2,3-d]pyrimidin-7(8H)-one
2-((4-((2-(dimethylamino)ethyl)(methyl)amino)phenyl)amino)-5-ethynyl-8-methylpyrido[2,3-d]pyrimidin-7(8H)-one
5-ethynyl-2-((2-methoxy-4-(4-methylpiperazin-1-yl)phenyl)amino)-8-methylpyrido[2,3-d]pyrimidin-7(8H)-one
N-((1s,4s)-4-(2-((4-((2-(dimethylamino)ethyl)(methyl)amino)phenyl)amino)-5-ethynyl-7-oxopyrido[2,3-d]pyrimidin-8(7H)-yl)cyclohexyl)acetamide
2-((4-((2-(dimethylamino)ethyl)(methyl)amino)phenyl)amino)-5-ethynyl-8-((1s,4s)-4-(hydroxymethyl)cyclohexyl)pyrido[2,3-d]pyrimidin-7(8H)-one
6-(2-chlorophenyl)-2-((4-((2-(dimethylamino)ethyl)(methyl)amino)-3-methylphenyl)amino)-5-ethynyl-8-methylpyrido[2,3-d]pyrimidin-7(8H)-one
8-(2,4-dimethoxyphenyl)-2-((4-((2-(dimethylamino)ethyl)(methyl)amino)phenyl)amino)-5-ethynylpyrido[2,3-d]pyrimidin-7(8H)-one
2-((4-((2-(dimethylamino)ethyl)(methyl)amino)-3-(1-methyl-1H-pyrazol-4-yl)phenyl)amino)-5-ethynyl-8-methylpyrido[2,3-d]pyrimidin-7(8H)-one
6-(2-chloro-5-methoxyphenyl)-2-((4-((2-(dimethylamino)ethyl)(methyl)amino)phenyl)amino)-5-ethynyl-8-methylpyrido[2,3-d]pyrimidin-7(8H)-one
6-(2-chloro-5-fluorophenyl)-2-((4-((2-(dimethylamino)ethyl)(methyl)amino)phenyl)amino)-5-ethynyl-8-methylpyrido[2,3-d]pyrimidin-7(8H)-one
5-ethynyl-8-methyl-2-((4-(4-methylpiperazin-1-yl)phenyl)amino)pyrido[2,3-d]pyrimidin-7(8H)-one
2-((4-(2-(dimethylamino)ethoxy)phenyl)amino)-5-ethynyl-8-phenylpyrido[2,3-d]pyrimidin-7(8H)-one
5-ethynyl-2-((2-methoxyphenyl)amino)pyrido[2,3-d]pyrimidin-7(8H)-one
8-(2,4-dimethoxyphenyl)-2-((4-((2-(dimethylamino)ethyl)(methyl)amino)phenyl)amino)-5-vinylpyrido[2,3-d]pyrimidin-7(8H)-one
6-(2-chloro-3-fluorophenyl)-2-((4-((2-(dimethylamino)ethyl)(methyl)amino)phenyl)amino)-5-ethynyl-8-methylpyrido[2,3-d]pyrimidin-7(8H)-one
2-((4-(4-(dimethylamino)piperidin-1-yl)phenyl)amino)-5-ethynyl-8-phenylpyrido[2,3-d]pyrimidin-7(8H)-one
2-((4-((2-(dimethylamino)ethyl)(methyl)amino)phenyl)amino)-5-ethynyl-6-(2-methoxyphenyl)-8-methylpyrido[2,3-d]pyrimidin-7(8H)-one
2-((4-((2-(dimethylamino)ethyl)(methyl)amino)phenyl)amino)-5-ethynyl-8-methyl-6-(phenylamino)pyrido[2,3-d]pyrimidin-7(8H)-one
5-ethynyl-8-phenyl-2-((4-(piperazin-1-yl)phenyl)amino)pyrido[2,3-d]pyrimidin-7(8H)-one
2-((4-(4-ethylpiperazin-1-yl)phenyl)amino)-5-ethynyl-8-phenylpyrido[2,3-d]pyrimidin-7(8H)-one
5-ethynyl-2-((4-(4-isopropylpiperazin-1-yl)phenyl)amino)-8-phenylpyrido[2,3-d]pyrimidin-7(8H)-one
5-ethynyl-2-((4-(4-(2-hydroxyethyl)piperazin-1-yl)phenyl)amino)-8-phenylpyrido[2,3-d]pyrimidin-7(8H)-one
5-ethynyl-2-((4-(4-(oxetan-3-yl)piperazin-1-yl)phenyl)amino)-8-phenylpyrido[2,3-d]pyrimidin-7(8H)-one
5-ethynyl-2-((4-(1-methylpiperidin-4-yl)phenyl)amino)-8-phenylpyrido[2,3-d]pyrimidin-7(8H)-one
2-((4-((2-(dimethylamino)ethyl)(methyl)amino)-3-methylphenyl)amino)-5-ethynyl-8-phenylpyrido[2,3-d]pyrimidin-7(8H)-one
6-(3-chloropyridin-4-yl)-2-((4-((2-(dimethylamino)ethyl)(methyl)amino)phenyl)amino)-5-ethynyl-8-methylpyrido[2,3-d]pyrimidin-7(8H)-one
2-((4-(4,7-diazaspiro[2.5]octan-7-yl)phenyl)amino)-5-ethynyl-8-phenylpyrido[2,3-d]pyrimidin-7(8H)-one
5-ethynyl-2-((1-(1-methylpiperidin-4-yl)-1H-pyrazol-4-yl)amino)-8-phenylpyrido[2,3-d]pyrimidin-7(8H)-one
2-((4-(1,4-diazepan-1-yl)phenyl)amino)-5-ethynyl-8-phenylpyrido[2,3-d]pyrimidin-7(8H)-one
5-ethynyl-8-phenyl-2-((4-(piperidin-4-yl)phenyl)amino)pyrido[2,3-d]pyrimidin-7(8H)-one
5-ethynyl-2-((4-(4-(4-methylpiperazin-1-yl)piperidin-1-yl)phenyl)amino)-8-phenylpyrido[2,3-d]pyrimidin-7(8H)-one
5-ethynyl-8-phenyl-2-((2,3,4,5-tetrahydro-1H-benzo[d]azepin-7-yl)amino)pyrido[2,3-d]pyrimidin-7(8H)-one
5-ethynyl-2-((4-(3-methylpiperazin-1-yl)phenyl)amino)-8-phenylpyrido[2,3-d]pyrimidin-7(8H)-one
8-(3-methoxyphenyl)-2-((2-methoxyphenyl)amino)-5-vinylpyrido[2,3-d]pyrimidin-7(8H)-one
5-ethynyl-2-((2-methoxyphenyl)amino)-8-(3-nitrophenyl)pyrido[2,3-d]pyrimidin-7(8H)-one
5-ethynyl-2-((2-methoxyphenyl)amino)-8-(4-nitrophenyl)pyrido[2,3-d]pyrimidin-7(8H)-one
8-(3-aminophenyl)-5-ethynyl-2-((2-methoxyphenyl)amino)pyrido[2,3-d]pyrimidin-7(8H)-one
5-ethynyl-2-((1-methyl-1H-pyrazol-4-yl)amino)-8-phenylpyrido[2,3-d]pyrimidin-7(8H)-one
5-ethynyl-2-((3-(hydroxymethyl)-4-(4-methylpiperazin-1-yl)phenyl)amino)-8-phenylpyrido[2,3-d]pyrimidin-7(8H)-one
5-ethynyl-2-((4-(4-(3-(hydroxymethyl)azetidin-1-yl)piperidin-1-yl)phenyl)amino)-8-phenylpyrido[2,3-d]pyrimidin-7(8H)-one
5-ethynyl-2-((4-(4-(3-fluoro-3-(hydroxymethyl)azetidin-1-yl)piperidin-1-yl)phenyl)amino)-8-phenylpyrido[2,3-d]pyrimidin-7(8H)-one
2-((4-(4-(3-(dimethylamino)azetidin-1-yl)piperidin-1-yl)phenyl)amino)-5-ethynyl-8-phenylpyrido[2,3-d]pyrimidin-7(8H)-one
5-ethynyl-2-((2-methoxy-5-(1-methyl-1H-pyrazol-4-yl)-4-morpholinophenyl)amino)-8-methylpyrido[2,3-d]pyrimidin-7(8H)-one
N-(5-((5-ethynyl-8-methyl-7-oxo-7,8-dihydropyrido[2,3-d]pyrimidin-2-yl)amino)-2-(4-methylpiperazin-1-yl)phenyl)acetamide
N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)propionamide
N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)isobutyramide
2-((3-(5-ethynyl-2-((2-methoxyphenyl)amino)-7-oxopyrido[2,3-d]pyrimidin-8(7H)-yl)phenyl)amino)-2-oxoethyl acetate
8-(4-aminophenyl)-5-ethynyl-2-((2-methoxyphenyl)amino)pyrido[2,3-d]pyrimidin-7(8H)-one
N-(4-(5-ethynyl-2-((2-methoxyphenyl)amino)-7-oxopyrido[2,3-d]pyrimidin-8(7H)-yl)phenyl)acetamide
2-((4-((3-(Dimethylamino)propyl)(methyl)amino)phenyl)amino)-5-ethynyl-8-phenylpyrido[2,3-d]pyrimidin-7(8H)-one
2-((3-ethyl-4-(4-methylpiperazin-1-yl)phenyl)amino)-5-ethynyl-8-phenylpyrido[2,3-d]pyrimidin-7(8H)-one
5-ethynyl-8-isopropyl-2-((4-(4-methylpiperazin-1-yl)phenyl)amino)pyrido[2,3-d]pyrimidin-7(8H)-one
8-cyclopentyl-5-ethynyl-2-((4-(4-methylpiperazin-1-yl)phenyl)amino)pyrido[2,3-d]pyrimidin-7(8H)-one
N-(5-((5-ethynyl-8-methyl-7-oxo-7,8-dihydropyrido[2,3-d]pyrimidin-2-yl)amino)-2-(4-methylpiperazin-1-yl)phenyl)propionamide
5-ethynyl-2-((1-(methylsulfonyl)piperidin-4-yl)amino)-8-phenylpyrido[2,3-d]pyrimidin-7(8H)-one
5-ethynyl-2-((4-morpholinophenyl)amino)-8-phenylpyrido[2,3-d]pyrimidin-7(8H)-one
N-(4-(5-ethynyl-2-((2-methoxyphenyl)amino)-7-oxopyrido[2,3-d]pyrimidin-8(7H)-yl)phenyl)methanesulfonamide
N-(4-(5-ethynyl-2-((2-methoxyphenyl)amino)-7-oxopyrido[2,3-d]pyrimidin-8(7H)-yl)phenyl)cyclopropanecarboxamide
5-ethynyl-2-((4-(4-(3-(methoxymethyl)azetidin-1-yl)piperidin-1-yl)phenyl)amino)-8-phenylpyrido[2,3-d]pyrimidin-7(8H)-one
N-((1r,4r)-4-(5-ethynyl-2-((4-(4-methylpiperazin-1-yl)phenyl)amino)-7-oxopyrido[2,3-d]pyrimidin-8(7H)-yl)cyclohexyl)acetamide
2-((4-(5-ethynyl-2-((2-methoxyphenyl)amino)-7-oxopyrido[2,3-d]pyrimidin-8(7H)-yl)phenyl)amino)-2-oxoethyl acetate
N-(4-(5-ethynyl-2-((2-methoxyphenyl)amino)-7-oxopyrido[2,3-d]pyrimidin-8(7H)-yl)phenyl)acrylamide
8-(4-methoxyphenyl)-2-((2-methoxyphenyl)amino)-5-vinylpyrido[2,3-d]pyrimidin-7(8H)-one
6-cyclopropyl-5-ethynyl-8-methyl-2-((4-(4-methylpiperazin-1-yl)phenyl)amino)pyrido[2,3-d]pyrimidin-7(8H)-one
5-ethynyl-6,8-dimethyl-2-((4-(4-methylpiperazin-1-yl)phenyl)amino)pyrido[2,3-d]pyrimidin-7(8H)-one
5-ethynyl-2-((4-(4-(3-(2-hydroxypropan-2-yl)azetidin-1-yl)piperidin-1-yl)phenyl)amino)-8-phenylpyrido[2,3-d]pyrimidin-7(8H)-one
2-((3-((2-(dimethylamino)ethyl)(methyl)amino)phenyl)amino)-5-ethynyl-8-methylpyrido[2,3-d]pyrimidin-7(8H)-one
N-(4-(5-ethynyl-2-((2-methoxyphenyl)amino)-7-oxopyrido[2,3-d]pyrimidin-8(7H)-yl)phenyl)propionamide
5-ethynyl-2-((2-(hydroxymethyl)phenyl)amino)-8-phenylpyrido[2,3-d]pyrimidin-7(8H)-one
5-ethynyl-8-methyl-2-((4-(4-methylpiperazin-1-yl)phenyl)amino)-6-(thiophen-2-yl)pyrido[2,3-d]pyrimidin-7(8H)-one
2-(dimethylamino)-N-(3-((5-ethynyl-8-methyl-7-oxo-7,8-dihydropyrido[2,3-d]pyrimidin-2-yl)amino)phenyl)-N-methylacetamide
N-((1r,4r)-4-(5-ethynyl-2-((4-(4-methylpiperazin-1-yl)phenyl)amino)-7-oxopyrido[2,3-d]pyrimidin-8(7H)-yl)cyclohexyl)pyridazine-4-carboxamide
2-(dimethylamino)-N-(4-((5-ethynyl-7-oxo-8-phenyl-7,8-dihydropyrido[2,3-d]pyrimidin-2-yl)amino)-3-methoxyphenyl)acetamide
4-(((1s,4s)-4-(5-ethynyl-2-((4-(4-methylpiperazin-1-yl)phenyl)amino)-7-oxopyrido[2,3-d]pyrimidin-8(7H)-yl)cyclohexyl)amino)-4-oxobutanoic acid
N1-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-N4,N4-dimethylsuccinamide
N1-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-N3-methylmalonamide
N1-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-N3,N3-dimethylmalonamide
3-(dimethylamino)-N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)propanamide
4-(dimethylamino)-N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)butanamide
N1-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-N4-methylsuccinamide
3-(((1s,4s)-4-(5-ethynyl-2-((4-(4-methylpiperazin-1-yl)phenyl)amino)-7-oxopyrido[2,3-d]pyrimidin-8(7H)-yl)cyclohexyl)amino)-3-oxopropanoic acid
2-(dimethylamino)-N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)acetamide
5-ethynyl-6-methyl-2-((4-(4-methylpiperazin-1-yl)phenyl)amino)-8-phenylpyrido[2,3-d]pyrimidin-7(8H)-one
8-(cyclopentylmethyl)-5-ethynyl-2-((4-(4-methylpiperazin-1-yl)phenyl)amino)pyrido[2,3-d]pyrimidin-7(8H)-one
5-ethynyl-2-((1′-methyl-[1,4′-bipiperidin]-4-yl)amino)-8-phenylpyrido[2,3-d]pyrimidin-7(8H)-one
8-cyclohexyl-5-ethynyl-2-((4-(4-methylpiperazin-1-yl)phenyl)amino)pyrido[2,3-d]pyrimidin-7(8H)-one
5-ethynyl-2-((4-(4-methylpiperazin-1-yl)phenyl)amino)-8-(tetrahydro-2H-pyran-4-yl)pyrido[2,3-d]pyrimidin-7(8H)-one
5-ethynyl-2-((4-(4-methylpiperazin-1-yl)phenyl)amino)-8-((tetrahydrofuran-3-yl)methyl)pyrido[2,3-d]pyrimidin-7(8H)-one
(S)-5-ethynyl-2-((4-(4-methylpiperazin-1-yl)phenyl)amino)-8-((5-oxopyrrolidin-2-yl)methyl)pyrido[2,3-d]pyrimidin-7(8H)-one
5-ethynyl-2-((4-(4-methylpiperazin-1-yl)phenyl)amino)-8-((tetrahydrofuran-2-yl)methyl)pyrido[2,3-d]pyrimidin-7(8H)-one
N-((1s,4s)-4-(5-ethynyl-2-((4-(4-methylpiperazin-1-yl)phenyl)amino)-7-oxopyrido[2,3-d]pyrimidin-8(7H)-yl)cyclohexyl)-2-(1H-pyrazol-4-yl)acetamide
2-(dimethylamino)-N-((1s,4s)-4-(5-ethynyl-2-((2-methoxyphenyl)amino)-7-oxopyrido[2,3-d]pyrimidin-8(7H)-yl)cyclohexyl)acetamide
5-ethynyl-8-(2-hydroxycyclopentyl)-2-((4-(4-methylpiperazin-1-yl)phenyl)amino)pyrido[2,3-d]pyrimidin-7(8H)-one
2-(dimethylamino)-N-((1s,4s)-4-(5-ethynyl-2-((2-methoxyphenyl)amino)-7-oxopyrido[2,3-d]pyrimidin-8(7H)-yl)cyclohexyl)-N-methylacetamide
N-((1r,4r)-4-(5-ethynyl-6-methyl-2-((4-(4-methylpiperazin-1-yl)phenyl)amino)-7-oxopyrido[2,3-d]pyrimidin-8(7H)-yl)cyclohexyl)acetamide
N-((1s,4s)-4-(5-ethynyl-6-methyl-2-((4-(4-methylpiperazin-1-yl)phenyl)amino)-7-oxopyrido[2,3-d]pyrimidin-8(7H)-yl)cyclohexyl)acetamide
5-ethynyl-2-((4-(4-methylpiperazin-1-yl)phenyl)amino)-8-(2-oxopyrrolidin-3-yl)pyrido[2,3-d]pyrimidin-7(8H)-one
5-ethynyl-2-((4-(4-methylpiperazin-1-yl)phenyl)amino)-8-(tetrahydrofuran-3-yl)pyrido[2,3-d]pyrimidin-7(8H)-one
5-ethynyl-2-((4-(4-methylpiperazin-1-yl)phenyl)amino)-8-(pyrrolidin-3-yl)pyrido[2,3-d]pyrimidin-7(8H)-one
5-ethynyl-2-((4-(4-methylpiperazin-1-yl)phenyl)amino)-8-(1-methylpyrrolidin-3-yl)pyrido[2,3-d]pyrimidin-7(8H)-one
5-ethynyl-2-((4-(4-methylpiperazin-1-yl)phenyl)amino)-8-(5-oxopyrrolidin-3-yl)pyrido[2,3-d]pyrimidin-7(8H)-one
8-((1H-pyrazol-5-yl)methyl)-5-ethynyl-6-methyl-2-((4-(4-methylpiperazin-1-yl)phenyl)amino)pyrido[2,3-d]pyrimidin-7(8H)-one
5-ethynyl-6-methyl-8-((1-methyl-1H-pyrazol-3-yl)methyl)-2-((4-(4-methylpiperazin-1-yl)phenyl)amino)pyrido[2,3-d]pyrimidin-7(8H)-one
8-((1H-imidazol-5-yl)methyl)-5-ethynyl-6-methyl-2-((4-(4-methylpiperazin-1-yl)phenyl)amino)pyrido[2,3-d]pyrimidin-7(8H)-one
5-ethynyl-6-methyl-8-((1-methyl-1H-pyrazol-5-yl)methyl)-2-((4-(4-methylpiperazin-1-yl)phenyl)amino)pyrido[2,3-d]pyrimidin-7(8H)-one
5-ethynyl-8-(isoxazol-5-ylmethyl)-6-methyl-2-((4-(4-methylpiperazin-1-yl)phenyl)amino)pyrido[2,3-d]pyrimidin-7(8H)-one
5-ethynyl-6-methyl-2-((4-(4-methylpiperazin-1-yl)phenyl)amino)-8-(pyridin-3-ylmethyl)pyrido[2,3-d]pyrimidin-7(8H)-one
8-(cyclopentylmethyl)-5-ethynyl-6-methyl-2-((4-(4-methylpiperazin-1-yl)phenyl)amino)pyrido[2,3-d]pyrimidin-7(8H)-one
5-ethynyl-8-(1-methyl-5-oxopyrrolidin-3-yl)-2-((4-(4-methylpiperazin-1-yl)phenyl)amino)pyrido[2,3-d]pyrimidin-7(8H)-one
5-ethynyl-8-((1-methyl-1H-pyrazol-3-yl)methyl)-2-((4-(4-methylpiperazin-1-yl)phenyl)amino)pyrido[2,3-d]pyrimidin-7(8H)-one
5-ethynyl-6-methyl-2-((4-(4-methylpiperazin-1-yl)phenyl)amino)-8-(oxazol-2-ylmethyl)pyrido[2,3-d]pyrimidin-7(8H)-one
5-ethynyl-2-((4-(4-methylpiperazin-1-yl)phenyl)amino)-8-((2-oxopyrrolidin-3-yl)methyl)pyrido[2,3-d]pyrimidin-7(8H)-one
1⁵-ethynyl-1⁷,1⁸-dihydro-4-oxa-2-aza-1(2,8)-pyrido[2,3-d]pyrimidina-3(1,3)-benzenacycloheptaphan-1⁷-one
(Z)-1⁵-ethynyl-1⁷,1⁸-dihydro-4-oxa-2-aza-1(2,8)-pyrido[2,3-d]pyrimidina-3(1,3)-benzenacyclooctaphan-6-en-1⁷-one
(Z)-1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-6-en-1⁷-one
1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-1⁷-one;
5-Ethynyl-6-methyl-2-{[4-(4-methylpiperazin-1-yl)phenyl]amino}-8-(1,2-oxazol-4-ylmethyl)pyrido[2,3-d]pyrimidin-7-one;
5-Ethynyl-6-methyl-2-{[4-(4-methylpiperazin-1-yl)phenyl]amino}-8-(1,3-oxazol-5-ylmethyl)pyrido[2,3-d]pyrimidin-7-one;
5-Ethynyl-8-(1H-imidazol-2-ylmethyl)-6-methyl-2-{[4-(4-methylpiperazin-1-yl)phenyl]amino}pyrido[2,3-d]pyrimidin-7-one;
5-Ethynyl-6-methyl-2-{[4-(4-methylpiperazin-1-yl)phenyl]amino}-8-(pyridin-2-ylmethyl)pyrido[2,3-d]pyrimidin-7-one;
5-Ethynyl-6-methyl-2-{[4-(4-methylpiperazin-1-yl)phenyl]amino}-8-(pyridin-4-ylmethyl)pyrido[2,3-d]pyrimidin-7-one;
5-Ethynyl-6-methyl-2-{[4-(4-methylpiperazin-1-yl)phenyl]amino}-8-[(1-methylpyrazol-4-yl)methyl]pyrido[2,3-d]pyrimidin-7-one;
8-Cyclopentyl-5-ethynyl-2-[(3-([3-(methanesulfonylmethyl)azetidin-1-yl]methylphenyl)amino]pyrido[2,3-d]pyrimidin-7-one;
8-Cyclopentyl-5-ethynyl-2-[(3-([3-(methanesulfonylmethyl)pyrrolidin-1-yl]methylphenyl)amino]pyrido[2,3-d]pyrimidin-7-one;
8-Cyclopentyl-5-ethynyl-2-[(3-([4-(methanesulfonylmethyl)piperidin-1-yl]methylphenyl)amino]pyrido[2,3-d]pyrimidin-7-one;
8-Cyclopentyl-5-ethynyl-2-[(2-methyl-1,3-dihydroisoindol-4-yl)amino]pyrido[2,3-d]pyrimidin-7-one;
8-Cyclopentyl-5-ethynyl-2-[(2-methyl-3,4-dihydro-1H-isoquinolin-5-yl)amino]pyrido[2,3-d]pyrimidin-7-one;
(Z)-1⁵-ethynyl-1⁶-methyl-1⁷,1⁸-dihydro-4-oxa-2-aza-1(2,8)-pyrido[2,3-d]pyrimidina-3(1,3)-benzenacyclooctaphan-6-en-1⁷-one:
1⁵-ethynyl-1⁶-methyl-1⁷,1⁸-dihydro-4-oxa-2-aza-1(2,8)-pyrido[2,3-d]pyrimidina-3(1,3)-benzenacyclooctaphan-1⁷-one;
(5¹R,5³S)-3⁵-ethynyl-3⁷,3³-dihydro-7-oxa-2-aza-3(2,8)-pyrido[2,3-d]pyrimidina-1(1,3)-benzena-5(1,3)-cyclopentanacycloheptaphan-3⁷-one;
1⁵-Ethynyl-1⁷,1⁸-dihydro-4-oxa-2-aza-1(2,8)-pyrido[2,3-d]pyrimidina-3(1,3)-benzenacyclooctaphan-1⁷-one;
1⁵-Ethynyl-1⁶-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-1⁷-one;
(Z)-1⁵-ethynyl-1⁷,1⁸-dihydro-5-oxa-2-aza-1(2,8)-pyrido[2,3-d]pyrimidina-3(1,3)-benzenacyclononaphan-7-en-1⁷-one;
1⁵-Ethynyl-34-(4-methylpiperazin-1-yl)-1⁷,1⁸-dihydro-5-oxa-2-aza-1(2,8)-pyrido[2,3-d]pyrimidina-3(1,3)-benzenacyclooctaphan-1⁷-one;
1⁵-Ethynyl-3⁴-(4-methylpiperazin-1-yl)-1⁷,1⁸-dihydro-2,6-diaza-1(2,8)-pyrido[2,3-d]pyrimidina-3(1,3)-benzenacyclooctaphane-1⁷,5-dione;
1⁵-Ethynyl-3⁴-(4-methylpiperazin-1-yl)-1⁷,1⁸-dihydro-2,4-diaza-1(2,8)-pyrido[2,3-d]pyrimidina-3(1,3)-benzenacyclooctaphan-1⁷-one;
1⁵-Ethynyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphan-1⁷-one;
1⁵-Ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclononaphan-17-one;
N-(5-((5-ethynyl-7-oxo-7,8-dihydropyrido[2,3-d]pyrimidin-2-yl)amino)-2-(4-methylpiperazin-1-yl)benzyl)acrylamide;
8-cyclopentyl-5-ethynyl-2-((2-(3-((methylsulfonyl)methyl)piperidin-1-yl)phenyl)amino)pyrido[2,3-d]pyrimidin-7(8H)-one;
8-cyclopentyl-5-ethynyl-2-((3-(methyl((1s,3s)-3-((methylsulfonyl)methyl)cyclobutyl)amino)phenyl)amino)pyrido[2,3-d]pyrimidin-7(8H)-one;
8-cyclopentyl-5-ethynyl-2-((3-(methyl((1s,4s)-4-((methylsulfonyl)methyl)cyclohexyl)amino)phenyl)amino)pyrido[2,3-d]pyrimidin-7(8H)-one;
8-cyclopentyl-5-ethynyl-2-((3-(methyl((1r,3r)-3-((methylsulfonyl)methyl)cyclobutyl)amino)phenyl)amino)pyrido[2,3-d]pyrimidin-7(8H)-one;
8-cyclopentyl-5-ethynyl-2-((3-(methyl((1r,4r)-4-((methylsulfonyl)methyl)cyclohexyl)amino)phenyl)amino)pyrido[2,3-d]pyrimidin-7(8H)-one;
5-ethynyl-8-methyl-2-((4-(4-methylpiperazin-1-yl)-3-propoxyphenyl)amino)pyrido[2,3-d]pyrimidin-7(8H)-one;
5-ethynyl-2-((3-methoxy-4-(4-methylpiperazin-1-yl)phenyl)amino)-8-methylpyrido[2,3-d]pyrimidin-7(8H)-one;
2-((3-(2-cyclopentylethoxy)-4-(4-methylpiperazin-1-yl)phenyl)amino)-5-ethynyl-8-methylpyrido[2,3-d]pyrimidin-7(8H)-one;
2-((3-(2-cyclopentylethoxy)-4-(4-methylpiperazin-1-yl)phenyl)amino)-5-ethynyl-8-((1-methyl-1H-pyrazol-3-yl)methyl)pyrido[2,3-d]pyrimidin-7(8H)-one;
2-((3-(cyclopentylmethoxy)-4-(4-methylpiperazin-1-yl)phenyl)amino)-5-ethynyl-8-methylpyrido[2,3-d]pyrimidin-7(8H)-one;
2-((3-(3-cyclopentylpropoxy)-4-(4-methylpiperazin-1-yl)phenyl)amino)-5-ethynyl-8-methylpyrido[2,3-d]pyrimidin-7(8H)-one;
2-((3-(2-cyclohexylethoxy)-4-(4-methylpiperazin-1-yl)phenyl)amino)-5-ethynyl-8-methylpyrido[2,3-d]pyrimidin-7(8H)-one;
5-ethynyl-8-methyl-2-((4-(4-methylpiperazin-1-yl)-3-phenethoxyphenyl)amino)pyrido[2,3-d]pyrimidin-7(8H)-one;
2-((3-(cyclopentyloxy)-4-(4-methylpiperazin-1-yl)phenyl)amino)-5-ethynyl-8-methylpyrido[2,3-d]pyrimidin-7(8H)-one;
5-ethynyl-2-((3-(isopentyloxy)-4-(4-methylpiperazin-1-yl)phenyl)amino)-8-methylpyrido[2,3-d]pyrimidin-7(8H)-one;
2-cyclopentyl-N-(5-((5-ethynyl-8-methyl-7-oxo-7,8-dihydropyrido[2,3-d]pyrimidin-2-yl)amino)-2-(4-methylpiperazin-1-yl)phenyl)acetamide;
N-(cyclopentylmethyl)-5-((5-ethynyl-8-methyl-7-oxo-7,8-dihydropyrido[2, 3-d]pyrimidin-2-yl)amino)-2-(4-methylpiperazin-1-yl)benzenesulfonamide;
1-cyclopentyl-3-(5-((5-ethynyl-8-methyl-7-oxo-7,8-dihydropyrido[2, 3-d]pyrimidin-2-yl)amino)-2-(4-methylpiperazin-1-yl)phenyl)urea;
5-ethynyl-8-methyl-2-((3-(2-(1-methyl-1H-pyrazol-3-yl)ethoxy)-4-(4-methylpiperazin-1-yl)phenyl)amino)pyrido[2,3-d]pyrimidin-7(8H)-one;
N-(cyclopentylmethyl)-5-((5-ethynyl-8-methyl-7-oxo-7,8-dihydropyrido[2,3-d]pyrimidin-2-yl)amino)-2-(4-methylpiperazin-1-yl)benzamide;
1⁵-ethynyl-3⁴-(4-methylpiperazin-1-yl)-1⁷,1⁸-dihydro-2,4-diaza-1(2,8)-pyrido[2,3-d]pyrimidina-3(1,3)-benzenacyclooctaphane-1⁷,5-dione;
1⁵-ethynyl-5-methyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphane-1⁷,6-dione;
1⁵-ethynyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphane-1⁷,4-dione;
1⁵-ethynyl-4-methyl-3⁴-(4-methylpiperazin-1-yl)-1⁷,1⁸-dihydro-2,4-diaza-1(2,8)-pyrido[2,3-d]pyrimidina-3(1,3)-benzenacyclooctaphane-1⁷,5-dione;
1⁵-ethynyl-5-methyl-3⁴-(4-methylpiperazin-1-yl)-1⁷,1⁸-dihydro-2,5-diaza-1(2,8)-pyrido[2,3-d]pyrimidina-3(1,3)-benzenacyclooctaphan-1⁷-one:
1⁵-ethynyl-3⁴-(4-methylpiperazin-1-yl)-1⁷,1⁸-dihydro-2,6-diaza-1(2,8)-pyrido[2,3-d]pyrimidina-3(1,3)-benzenacyclooctaphane-1⁷,7-dione;
1⁵-ethynyl-9-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclononaphan-1⁷-one;
1⁵-ethynyl-16-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclononaphan-1⁷-one;
1⁵-ethynyl-3⁴-(4-methylpiperazin-1-yl)-1⁷,1⁸-dihydro-4-oxa-2-aza-1(2,8)-pyrido[2,3-d]pyrimidina-3(1,3)-benzenacyclodecaphan-1⁷-one;
1⁵-ethynyl-3⁴-(4-methylpiperazin-1-yl)-1⁷,1⁸-dihydro-5-oxa-2-aza-1(2,8)-pyrido[2,3-d]pyrimidina-3(1,3)-benzenacyclononaphan-1⁷-one;
1⁵-ethynyl-3⁴-(4-methylpiperazin-1-yl)-1⁷,1⁸-dihydro-4,7-dioxa-2-aza-1(2,8)-pyrido[2,3-d]pyrimidina-3(1,3)-benzenacyclononaphan-1⁷-one;
(5¹S,5³R)-3⁵-ethynyl-1⁴-(4-methylpiperazin-1-yl)-3⁷,3⁸-dihydro-7-oxa-2-aza-3(2,8)-pyrido[2,3-d]pyrimidina-1(1,3)-benzena-5(1,3)-cyclopentanacycloheptaphan-3⁷-one;
1⁵-ethynyl-3⁴-(4-methylpiperazin-1-yl)-1⁷,1⁸-dihydro-4-thia-2,5-diaza-1(2,8)-pyrido[2,3-d]pyrimidina-3(1,3)-benzenacyclooctaphan-1⁷-one 4,4-dioxide;
1⁵-ethynyl-3⁴-(4-isopropylpiperazin-1-yl)-1⁷,1⁸-dihydro-2,4-diaza-1(2,8)-pyrido[2,3-d]pyrimidina-3(1,3)-benzenacyclooctaphane-1⁷,5-dione;
1⁵-ethynyl-3⁴-(4-(2-methoxyethyl)piperazin-1-yl)-1⁷,1⁸-dihydro-2,4-diaza-1(2,8)-pyrido[2,3-d]pyrimidina-3(1,3)-benzenacyclooctaphane-1⁷,5-dione;
1⁵-ethynyl-3⁴-(4-methylpiperazin-1-yl)-1⁷,1⁸-dihydro-2,5-diaza-1(2,8)-pyrido[2,3-d]pyrimidina-3(1,3)-benzenacyclononaphane-1⁷,6-dione;
1⁵-ethynyl-3⁴-(4-methyl-1,4-diazepan-1-yl)-1⁷,1⁸-dihydro-2,4-diaza-1(2,8)-pyrido[2,3-d]pyrimidina-3(1,3)-benzenacyclooctaphane-1⁷,5-dione;
(R)-1⁵-ethynyl-8-methyl-3⁴-(4-methylpiperazin-1-yl)-1⁷,1⁸-dihydro-4-oxa-2-aza-1(2,8)-pyrido[2,3-d]pyrimidina-3(1,3)-benzenacyclooctaphan-1⁷-one;
1⁵-ethynyl-3⁴-(4-methylpiperazin-1-yl)-1⁷,1⁸-dihydro-6-oxa-2-aza-1(2,8)-pyrido[2,3-d]pyrimidina-3(1,3)-benzenacyclooctaphan-1⁷-one; and
1⁵-ethynyl-3⁴-(4-(2-methoxyethyl)piperazin-1-yl)-1⁷,1⁸-dihydro-4-oxa-2-aza-1(2,8)-pyrido[2,3-d]pyrimidina-3(1,3)-benzenacyclooctaphan-1⁷-one.

25. A pharmaceutical composition comprising a compound according to any one of claims 1 to 24, or a pharmaceutically acceptable salt, hydrate or solvate thereof, and a pharmaceutically acceptable excipient.

26. A compound according to any one of claims 1 to 24, or a pharmaceutically acceptable salt thereof, or a pharmaceutical composition according to claim 25 for use in:
(i) therapy;
(ii) the treatment of a disease or condition in which EGFR activity is implicated;
(iii) the treatment of a disease or condition associated with aberrant activity of EGFR;
(iv) the treatment of proliferative disorders (e.g. cancer or benign neoplasms);
(v) the treatment of a cancer;
(vi) the treatment of an EGFR positive cancer, optionally selected from head and neck cancer, brain cancer, breast cancer, colon cancer and/or lung cancer;
(vii) EGFR positive non-small cell lung cancer;
(viii) the treatment of a cancer expressing a mutated form of EGFR (for example EGFR comprising a T790M mutation, a deletion in exon 19 (e.g. A740-A750), an exon 20 insertion, a mutation at L858R and/or a C797S mutation);
(ix) the treatment of a cancer resistant to treatment with a third generation EFGR inhibitor, e.g. osimertinib, lazertinib (YH25448), EGF816, olmutinib, PF-06747775, avitinib and/or rociletinib;
(x) the treatment of non-small cell lung cancer resistant to treatment with osimertinib.
